(19) United States
(12) Patent Application Publication
(10) Pub. No.: US 2024/0309320 A1
(43) Pub. Date: Sep. 19, 2024

(54) METHODS FOR DIFFERENTIATING AND SCREENING STEM CELLS

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Julia Joung, Cambridge, MA (US)

(21) Appl. No.: 18/576,909

(22) PCT Filed: Jul. 8, 2022
(86) PCT No.: PCT/US2022/073548

§ 371 Date: Jan. 5, 2024

Related U.S. Application Data
(60) Provisional application No. 63/219,705, filed on Jul. 8, 2021.
(60) Provisional application No. 63/313,842, filed on Feb. 25, 2022.

(51) Int. Cl. C12N 5/0793; A61K 35/30; C12Q 1/6809; C12Q 1/6881

(52) U.S. Cl. CPC C12N 5/0619; A61K 35/30; C12Q 1/6809; C12Q 1/6881; C12N 2510/00; C12Q 2600/158

(57) ABSTRACT

The subject matter disclosed herein is generally directed to methods of differentiating pluripotent cells into target cell types and screening platforms for systematically identifying transcription factors (TFs) that drive differentiation of pluripotent cells into target cell types. Also disclosed is a high-throughput multiplex screening platform. Also disclosed are in vitro models for neural progenitor cells and cardiomyocytes.

### CROSS-REFERENCE TO RELATED APPLICATIONS

This application claims the benefit of U.S. Provisional Application Nos. 63/219,705, filed Jul. 8, 2021 and 63/313,842, filed Feb. 25, 2022. The entire contents of the above-identified applications are hereby fully incorporated herein by reference.

### STATEMENT REGARDING FEDERALLY SPONSORED RESEARCH

This invention was made with government support under Grant Nos. MH117886, HG009761, MH110049, and HL141201 awarded by the National Institutes of Health. The government has certain rights in the invention.

### REFERENCE TO AN ELECTRONIC SEQUENCE LISTING

The contents of the electronic sequence listing (BROD-5420WP_ST26.xml”; Size is 23,452,824 (23.5 MB on disk) bytes and it was created on Jul. 8, 2022) is herein incorporated by reference in its entirety.

### TECHNICAL FIELD

The subject matter disclosed herein is generally directed to methods of differentiating stem cells into target cell types and screening platforms for systematically identifying transcription factors (TFs) that drive differentiation of stem cells into target cell types.

### BACKGROUND

Directed differentiation of human pluripotent stem cells into diverse cell types has the potential to realize a broad array of cellular replacement therapies and provides a tractable model that can be perturbed, genetically or chemically, to assess effects in a cell type-specific context¹⁻⁵. Despite the utility of cellular engineering, however, it remains challenging or impossible to generate many cell types¹⁻⁵. The best differentiation methods are often labor-intensive and can require months to produce even heterogenous or immature cell populations. Many of these methods rely on exogenous growth factors or small molecules, which are often dosage-sensitive and difficult to identify in a scalable manner. Alternatively, overexpression of transcription factors (TFs) has been shown to rapidly and efficiently generate many different cell types, including neurons and skeletal muscle cells⁶⁻¹². As TFs use endogenous regulatory pathways to drive differentiation, mimicking natural development, this approach to engineering cell fate may produce higher fidelity models while illuminating aspects of cellular development. However, the process of discovering TFs for directed differentiation relies on time-intensive and low-throughput arrayed screens. Arrayed screens, in which each perturbation must be performed and tested individually, are inherently limited in their scalability, typically 5-25 TFs⁶⁻¹². By contrast, pooled screening approaches, which make use of barcodes to enable multiple perturbations to be tested in parallel, are dramatically more scalable, both in terms of time and cost.

In vitro models of the human brain enable high-throughput genetic and chemical screens that can advance our understanding of complex neuro-developmental and -degenerative diseases. To simultaneously assess thousands of different perturbations and ensure unbiased results, such models should be homogenous, robust, and scalable. Current methods for generating models of the brain generally involve differentiating human embryonic stem cells (hESCs) into neural cells using exogenous factors or small molecules, a process that is labor-intensive, time-consuming, and produces non-homogeneous cell types (Douvaras P, et al., Efficient generation of myelinating oligodendrocytes from primary progressive multiple sclerosis patients by induced pluripotent stem cells. Stem Cell Reports. 2014; 3(2):250-9; Krencik R, et al., Specification of transplantable astroglial subtypes from human pluripotent stem cells. Nat Biotechnol. 2011; 29(6):528-34; Li X J, et al., Specification of motoneurons from human embryonic stem cells. Nat Biotechnol. 2005; 23(2):215-21; Perrier A L, et al., Derivation of midbrain dopamine neurons from human embryonic stem cells. Proc Natl Acad Sci USA. 2004; and Muffat J, et al., Efficient derivation of microglia-like cells from human pluripotent stem cells. Nat Med. 2016; 22(11):1358-67). Furthermore, many cell types in the brain cannot be derived from hESCs. Although methods exist for differentiating neural progenitors and some neuronal subtypes, none efficiently generate glial cells (astrocytes, oligodendrocytes, and microglia) that resemble their in vivo counterparts without transplantation (Douvaras P, et al., 2014, Krencik R, et al., 2011, and Muffat J, et al., 2016). Since glia have been shown to play critical roles in neural development and disease, including them in models is critical to the success of this approach for studying the brain (Chung W S, et al., Do glia drive synaptic and cognitive impairment in disease? Nat Neurosci. 2015; 18(11): 1539-45; and Hong S, Stevens B. Microglia: Phagocytosing to Clear, Sculpt, and Eliminate. Dev Cell. 2016; 38(2):126-8).

Thus, there is a need to develop an efficient method that can generate more complete in vitro models of the human brain. Additionally, there is a need for in vitro models of other cell types that can advance our understanding of development and disease.

### SUMMARY

In certain example embodiments, the present invention provides for screening platforms for systematically identifying transcription factors (TFs) that drive differentiation of pluripotent stem cells into target cell types. In certain example embodiments, the present invention provides for differentiation methods based on overexpression of TFs to generate specific cell types. Applicants provide examples of the screening methods to identify transcription factors that are capable of differentiating stem cells into all cell types, including neural progenitors/radial glia in the developing central nervous system that are capable of differentiating into neurons, astrocytes, and oligodendrocytes. In certain embodiments, the neural progenitors are referred to as induced neural progenitors (iNPs). Some, but not all, of the iNPs become radial glial cells. Thus, “neural progenitors” as used herein may be referred to as “induced neural progenitors” or “radial glia”. Applicants further identify TFs that are capable of differentiating stem cells into cardiomyocytes.

In one aspect, the present invention provides for a method of differentiating a pluripotent cell population to a target cell type of interest comprising overexpressing one or more transcription factors (TFs) from Table 1 or Table 3 in a pluripotent cell population, and selecting cells expressing one or more target cell markers. In certain embodiments, the target cell is a neural progenitor and selecting cells comprises selecting cells expressing one or more radial glial cell markers. In certain embodiments, the one or more transcription factors are selected from the group consisting of RFX4, NFIB, ASCL1, PAX6, EOMES, FOS, OTX1, NFIC, LHX2, FANCD2, NOTCH1, SMARCC1, ESR2, ESR1, MESP1, RCOR2, GLI3, NOTCH2, HELLS, BCL11A, HES1, FANCD2, SOX9, FEZF2, and TCF7L2 or TFs that are ranked in the top 10% of any screening method in Table 1 (e.g., RFX4, NFIB, ASCL1, PAX6, EOMES, FOS, OTX1, NFIC, LHX2, RCOR2, GLI3, NOTCH2, HELLS, BCL11A, HES1, FANCD2, SOX9, FEZF2, TCF7L2). In certain embodiments, the one or more transcription factors are RFX4, NFIB, ASCL1, PAX6, or a combination thereof. In preferred embodiments, RFX4 is overexpressed to produce the neural progenitors. In certain embodiments, the method further comprises producing RFX4 neural progenitor cells in media comprising dual SMAD inhibitors. In certain embodiments, the one or more radial glial cell markers are selected from Table 2. In certain embodiments, the one or more radial glial cell markers are selected from the group consisting of NES, VIM, SLC1A3, and PAX6. In certain embodiments, the method further comprises inducing differentiation of the neural progenitors into neurons, astrocytes and/or oligodendrocytes. In certain embodiments, differentiation comprises spontaneous differentiation of the neural progenitors. In certain embodiments, differentiation comprises directed differentiation of the neural progenitors.

In certain embodiments, selecting further comprises selecting cells enriched for expression of one or more gene signatures expressed in in vivo radial glia cells. The one or more gene signatures may be any in vivo gene signature known in the art (see, e.g., Pollen et al., Molecular identity of human outer radial glia during cortical development. Cell. 2015; 163(1):55-67). In certain embodiments, selecting cells enriched for expression of one or more gene signatures expressed in in vivo radial glia cells comprises identifying gene signatures for each TF by identifying differentially expressed genes between cells overexpressing a transcription factor and control cells; and selecting cells having a signature that is enriched in an in vivo radial glia cell type. Differentially expressed genes may be identified by comparing expression of genes in cells overexpressing a transcription factor and control cells overexpressing only the reporter gene (e.g., GFP). In certain embodiments, the signature may encompass the top differentially expressed genes (e.g., top 10, 100, 1000 or more most differentially expressed genes). In certain embodiments, the gene signatures are compared to in vivo cells and the gene signatures from cells having an overexpressed transcription factor that are most enriched in the in vivo cell types are selected.

In another aspect, the present invention provides for an isolated neural progenitor cell produced by the method of any embodiment herein. In certain embodiments, the present invention provides for a therapeutic composition comprising the isolated neural progenitor cell. In certain embodiments, the present invention provides for an ex vivo system comprising the isolated neural progenitor cell.

In another aspect, the present invention provides for a method of producing neurons, astrocytes and/or oligodendrocytes comprising expressing one or more transcription factors from Table 1 in the isolated neural progenitor cell of any embodiment herein and inducing spontaneous differentiation of the isolated neural progenitor cells. In another aspect, the present invention provides for a method of producing neurons, astrocytes and/or oligodendrocytes comprising expressing one or more transcription factors from Table 1 in the isolated neural progenitor cell of any embodiment herein and inducing directed differentiation of the isolated neural progenitor cells. In preferred embodiments, the neural progenitor cell was produced by overexpression of RFX4. In certain embodiments, the method further comprises differentiating RFX4 neural progenitor cells in media comprising dual SMAD inhibitors. In certain embodiments, the RFX4 neural progenitor cells are differentiated for 7 days. In certain embodiments, the RFX4 neural progenitor cells are differentiated into CNS cell types, radial glia, and neurons. In certain embodiments, the neurons are GABAergic neurons.

In another aspect, the present invention provides for an isolated neuron, astrocyte, or oligodendrocyte produced according to any method described herein. In certain embodiments, the present invention provides for a therapeutic composition comprising the isolated neuron, astrocyte, or oligodendrocyte. In certain embodiments, the present invention provides for an ex vivo system comprising the isolated neurons, astrocytes, and/or oligodendrocytes. In preferred embodiments, the neuron is a GABAergic neuron. In certain embodiments, the GABAergic neuron can be used in a model of autism, schizophrenia, epilepsy, dementia, Alzheimer's disease, or anxiety disorders (e.g., depression).

In another aspect, the present invention provides for a non-naturally occurring population of stem cells comprising a reporter gene integrated into an endogenous locus of each stem cell in the population, wherein the endogenous locus is associated with a marker gene for a cell type of interest; the reporter gene is under control of the promoter for the marker gene; and the reporter gene and marker gene are expressed as separate proteins, whereby the marker gene and reporter gene are co-expressed upon differentiation of the stem cells into the cell type of interest. The non-naturally occurring population of stem cells may further comprise a second reporter gene integrated into a second endogenous locus of the stem cell, wherein the locus is associated with a marker gene for a second cell type of interest, and wherein the second cell type of interest is more differentiated than the first cell type of interest. The reporter gene and marker gene (e.g., first and/or second) may be separated by a ribosomal skipping site. The ribosomal skipping site may be a P2A sequence. The reporter gene may be a fluorescent protein as described herein. The cell type of interest may be any differentiated cell (e.g., more differentiated than a stem cell, including but not limited to a progenitor cell). The cell type of interest may be a neural progenitor or mature neural cell type.

In certain embodiments, the cell type of interest is a radial glia cell. The marker gene may be selected from Table 2. The marker gene may be selected from the group consisting of NES, VIM, SLC1A3, and PAX6.

In certain embodiments, the cell type of interest is an astrocyte. The marker gene may be selected from the group consisting of ALDH1L1 and GFAP.

In another aspect, the present invention provides for a pooled transcription factor screening system comprising a transcription factor library comprising one or more vectors encoding a transcription factor and a barcode identifying said transcription factor; and a population of pluripotent cells. In certain embodiments, the transcription factors encoded by the vectors are selected from Table 1 and/or Table 3. In certain embodiments, the population of pluripotent cells are stem cells. In certain embodiments, the system further comprises one or more fluorescent probes configured for detecting one or more target cell marker gene transcripts (e.g., Flow-FISH probes).

In another aspect, the present invention provides for a method of screening for transcription factors capable of differentiating pluripotent cells into a cell type of interest comprising: a) introducing a transcription factor library comprising one or more vectors to a population of pluripotent cells, wherein each vector encodes: a transcription factor selected from Table 1 and/or Table 3 or an agent capable of modulating said transcription factor, and a barcode identifying each transcription factor; b) culturing the cells to allow differentiation of the cells (e.g., 2-10 days, or 2-7 days, or 5-7 days); c) selecting cells expressing one or more marker genes for the cell type of interest; and d) determining barcodes enriched in cells expressing the one or marker genes, thereby identifying transcription factors capable of differentiating pluripotent cells into a cell type of interest. In certain embodiments, the population of pluripotent cells is a population of human embryonic stem cells (hESCs). In certain embodiments, each transcription factor is inducible. In certain embodiments, the transcription factors selected are normally expressed by the cell type of interest.

In certain embodiments, selecting cells expressing one or more marker genes for the cell type of interest comprises Flow-FISH using probes targeting one or more marker genes. In certain embodiments, selecting cells expressing one or more marker genes for the cell type of interest comprises single cell RNA-seq. In certain embodiments, selecting cells further comprises comparing single cell RNA-seq expression profiles of cells overexpressing one or more of the transcription factors to those of cells overexpressing controls (e.g., green fluorescent protein) to infer pseudotime for each cell, wherein transcription factors that increased pseudotimes direct differentiation. In certain embodiments, selecting cells further comprises grouping one or more of the transcription factors in modules that alter expression of the same gene programs, wherein transcription factors in the same modules are co-functional.

In certain embodiments, the one or more populations of pluripotent cells are stem cells. In certain embodiments, selecting cells expressing one or marker genes for the cell type of interest comprises detecting the reporter gene. In certain embodiments, selecting cells comprises FACS.

In certain embodiments, determining barcodes comprises sequencing the DNA barcode or transcript comprising the barcode. In certain embodiments, determining barcodes comprises amplification of barcode sequences (e.g., PCR).

In certain embodiments, the method further comprises introducing the transcription factor library at a low cell density, such that the cells multiply into small colonies; and inducing expression of the transcription factors or agents encoded by the vectors. In certain embodiments, the method further comprises introducing the vector library at a low MOI, such that most cells receive no more than one vector. In certain embodiments, the method further comprises introducing the vector library at a high MOI, such that most cells receive one or more vectors.

In certain embodiments, the transcription factor library comprises viral vectors. In certain embodiments, the viral vectors are lentivirus, adenovirus or adeno associated virus (AAV) vectors.

In certain embodiments, the transcription factor library further encodes a protein tag in frame with the transcription factor coding sequence.

In certain embodiments, the population of stem cells expresses a CRISPR system and the transcription factor library comprises vectors encoding one or more CRISPR guide sequences targeting one of the transcription factors. In certain embodiments, the guide sequences comprise one or more aptamer sequences specific for binding an adaptor protein and the CRISPR system comprises an enzymatically inactive CRISPR enzyme and the adaptor protein comprises a functional domain. In certain embodiments, the CRISPR system comprises an enzymatically inactive CRISPR enzyme and a functional domain. In certain embodiments, the functional domain is a transcription activation or repression domain.

In certain embodiments, the transcription factor library comprises vectors encoding a shRNA for one of the transcription factors.

In certain embodiments, identifying transcription factors further comprises determining gene signatures for each identified TF, wherein the gene signature comprises differentially expressed genes between cells overexpressing each transcription factor and control cells; and selecting transcription factors inducing a gene signature that is enriched in an in vivo cell type.

In another aspect, the present invention provides for a method of producing cardiomyocytes comprising overexpressing a transcription factor selected from the group consisting of MESP1, EOMES and ESR1 in a pluripotent cell population, and selecting cells expressing one or more cardiomyocyte markers. In certain embodiments, the transcription factor is EOMES. In certain embodiments, the amino acid sequence of EOMES is SEQ ID NO: 10807 or SEQ ID NO: 10808. In certain embodiments, the transcription factor is induced for about 2 days. In certain embodiments, the transcription factor is induced when the cell density is about 500,000 cells/ml. In certain embodiments, the one or more cardiomyocyte markers comprises TNNT2. In certain embodiments, selecting further comprises selecting cells enriched for expression of one or more gene signatures expressed in in vivo cardiomyocytes.

In another aspect, the present invention provides for an isolated cardiomyocyte produced by the method according to any embodiment herein. In certain embodiments, the present invention provides for a therapeutic composition comprising the isolated cardiomyocyte. In certain embodiments, the present invention provides for an ex vivo system comprising the isolated cardiomyocyte.

In certain embodiments, the pluripotent cell according to any embodiment herein is an embryonic stem cell (ES) or induced pluripotent stem cell. In certain embodiments, the stem cell is a human embryonic stem cell (ES). In certain embodiments, the human embryonic stem cell is selected from the group consisting of HUES66, HUES64, HUES3, HUES8, HUES53, HUES28, HUES49, HUES9, HUES48, HUES45, HUES1, HUES44, HUES6, H1, HUES62, HUES65, H7, HUES13, H9, and HUES63. In certain embodiments, the stem cell is a human induced pluripotent stem cell (iPSC). In certain embodiments, the human iPSC is selected from the group consisting of 11a, PGP1, GM08330 (also known as GM8330-8), and Mito 210.

In another aspect, the present invention provides for a stem cell comprising an exogenous nucleotide sequence capable of inducible expression of one or more transcription factors selected from the group consisting of RFX4, NFIB, ASCL1 and PAX6.

In another aspect, the present invention provides for a stem cell comprising an exogenous nucleotide sequence capable of inducible expression of one or more transcription factors selected from the group consisting of MESP1, EOMES and ESR1.

In another aspect, the present invention provides for a method of predicting transcription factor combinations for differentiating a stem cell into a cell type of interest comprising determining the average gene expression of one or more genes for two or more stem cells each expressing a single transcription factor and comparing the average expression to a gene signature specific for the cell type of interest. In certain embodiments, the method further comprises differentiating a stem cell into the cell type of interest by expressing in the stem cell a double or triple combination of transcription factors whose average gene expression is most similar to a gene signature specific for the cell type of interest.

In another aspect, the present invention provides for a method of differentiating a stem cell into a cell type of interest comprising expressing in the stem cell a double or triple combination of transcription factors selected from the clusters in Table 19.

These and other aspects, objects, features, and advantages of the example embodiments can become apparent to those having ordinary skill in the art upon consideration of the following detailed description of illustrated example embodiments.

### BRIEF DESCRIPTION OF THE DRAWINGS

An understanding of the features and advantages of the present invention can be obtained by reference to the following detailed description that sets forth illustrative embodiments, in which the principles of the invention may be utilized, and the accompanying drawings of which:

FIG. 1—Targeted arrayed TF screen. (A), Screening schematic. (B), Expression of radial glia marker genes after ASCL1 overexpression. (C), Image of differentiated cells after 4 days of ASCL1 overexpression. Scale bar, 100 μm.

FIG. 2—Gene expression signature of differentiated radial glia. Heat map of Z-scores indicating enrichment of TF candidate gene expression signatures in each cell type in vivo.

FIG. 3—Immunostaining of radial glia differentiated from candidate TFs. (A), Immunostaining of radial glia markers (VIM and NES) after 12 days of TF overexpression. (B), Immunostaining of neurons (MAP2), astrocytes (GFAP), and oligodendrocytes (NG2) after 4 weeks of spontaneous differentiation from radial glia induced by candidate TF overexpression. Scale bar, 50 μm.

FIG. 4—Immunostaining of neurons and astrocytes differentiated from ASCL1. Immunostaining for markers identifying neurons (MAP2), astrocytes (GFAP), and oligodendrocyte precursors (NG2 and PDGFRA) at indicated time points after induction of the TF (7 days, 14 days, 28 days).

FIG. 5—Immunostaining of neurons and astrocytes differentiated from NFIB. Immunostaining for markers identifying neurons (MAP2), astrocytes (GFAP), and oligodendrocyte precursors (NG2 and PDGFRA) at indicated time points after induction of the TF (7 days, 14 days, 28 days).

FIG. 6—Immunostaining of neurons and astrocytes differentiated from PAX6. Immunostaining for markers identifying neurons (MAP2), astrocytes (GFAP), and oligodendrocyte precursors (NG2 and PDGFRA) at indicated time points after induction of the TF (7 days, 14 days, 28 days).

FIG. 7—Immunostaining of neurons and astrocytes differentiated from RFX4. Immunostaining for markers identifying neurons (MAP2), astrocytes (GFAP), and oligodendrocyte precursors (NG2 and PDGFRA) at indicated time points after induction of the TF (7 days, 14 days, 28 days).

FIG. 8—Pooled TF screen. (A), Screening schematic. (B), Heat map of Z-scores representing median enrichment of each TF from 3 screens of 90 transcription factors performed in different clonal cell lines.

FIG. 9—Scatter Plot. Results of pooled screening of 1,387 transcription factors.

FIG. 10—Genome-wide astrocyte differentiation screen. Screening schematic.

FIG. 11—Cardiomyocyte differentiation. Bar graph showing the percentage of TNNT2 positive cells after cardiomyocyte differentiation of human embryonic stem cells under different conditions for inducing expression of two isoforms of EOMES.

FIG. 12—Cardiomyocyte differentiation. Bar graph showing the percentage of TNNT2 positive cells after cardiomyocyte differentiation of human embryonic stem cells under different conditions for inducing expression of two isoforms of EOMES or a small molecule differentiation method.

FIG. 13—Development of a pooled TF screening platform for directed differentiation. (A) Schematic of pooled TF screening. Barcoded TF ORFs are pooled and packaged into lentivirus for delivery into hESCs. TFs that can differentiate hESCs into the cell type of interest are identified using a reporter cell line, flow-FISH, or single-cell RNA sequencing, followed by deep sequencing of TF barcodes. MOI, multiplicity of infection. (B) Scatterplot showing enrichment of candidate TFs identified by flow-FISH with pooled FISH probes targeting 2 or 10 NP marker genes from n=3 infection replicates. (C) Same as (B) highlighting different isoforms of candidate TFs. (D) Comparison of TFs that ranked in the top 10% from the 4 different screens.

FIG. 14—Validation of candidate TFs for iNP differentiation. (A) Expression of NP marker genes VIM and NES in iNPs produced by candidate TFs after 7 days of overexpression. Cell culture media used for each ORF is indicated in parentheses. Scale bar, 50 μm. (B) Heat map of bulk RNA sequencing (RNA-seq) signature correlation between iNPs and human fetal cortex cell types from the Pollen 2015 dataset²⁰. D7 and D12 indicate the number of days that the ORF was overexpressed. RG, radial glia; IPC, intermediate progenitor cell; N, neuron; IN, interneuron.

FIG. 15—Candidate TFs produce iNPs that can spontaneously differentiate into cell types in the central nervous system. (A) Schematic of spontaneous differentiation. Dox-inducible candidate TFs are transiently overexpressed for 1 week to differentiate hESCs into iNPs and spontaneously differentiated for 8 weeks by withdrawing dox and growth factors. Spontaneously differentiated cells were characterized by immunostaining and single-cell RNA sequencing. rtTA, reverse tetracycline-controlled transactivator; dox, doxycycline; EGF, epidermal growth factor; FGF, fetal growth factor. (B) Expression of marker genes for neurons (MAP2), astrocytes (GFAP), and oligodendrocyte precursor cells (PDGFRA) after 1, 2, 4, or 8 weeks of spontaneous differentiation for 4 candidate TFs. Scale bar, 100 μm.

FIG. 16—Single-cell RNA sequencing of spontaneously differentiated cells from iNPs demonstrates development of a broad range of cell types. (A)-(C), t-distributed stochastic neighbor embedding (tSNE) visualization of single-cell RNA sequencing data from cells that have been spontaneously differentiated from iNPs for 8 weeks. iNPs were derived using RFX4, NFIB, ASCL1, or PAX6. A total of 52,364 cells from n=2 bioreps per TF were analyzed. (A) Cells are grouped into 31 clusters, and cluster 5 is further divided into 3 subclusters. Colors indicate cell type or state. (B) Clusters that represent central nervous system (CNS) cell types are highlighted. Percentage of total cells that contribute to the specified CNS cell type is indicated. (C) Cells spontaneously differentiated from each candidate TF are highlighted. Colors indicate bioreps, S1 and S2. (D) Quantification of spontaneously differentiated cells. Left, percentage of cells from each biorep that were grouped into each cluster. Right, over all distribution of general cell types. RP, retinal progenitors; RPE, retinal pigment epithelium; RGC, retinal ganglion cells; PR, photoreceptors; DNP, dorsal neural progenitors; RG, radial glia; Astro, astrocytes; CN, cortical neurons; HB&SCN, hindbrain and spinal cord neurons; IN, interneurons; EPD&CPE, ependyma and choroid plexis epithelium; EP, epithelial progenitors; BE, bronchial epithelium; CE, cranial epithelium; NC, neural crest; CNC, cranial neural crest; Pro, uncommitted progenitors; (P), proliferative cells; (S), structural cell types such as bone and cartilage.

FIG. 17—Modeling neurodevelopmental disorders using RFX4-iNPs with DYRK1A perturbation. (A) Schematic of disease modeling by perturbing DYRK1A expression. hESCs are transduced with Cas9 and DYRK1A KO sgRNAs or DYRK1A ORF to knockout or overexpress DYRK1A respectively. RFX4 is then transiently overexpressed for 1 week to differentiate hESCs into iNPs and spontaneously differentiated for 8 weeks by withdrawing dox and growth factors. Effects of DYRK1A perturbation were characterized by bulk RNA sequencing, EdU labeling, and immunostaining. rtTA, reverse tetracycline-controlled transactivator; dox, doxycycline; EGF, epidermal growth factor; FGF, fetal growth factor. (B)-(C), Expression of DYRK1A at 7 days after transduction with Cas9 and DYRK1A KO sgRNAs (B) or DYRK1A ORF (C). (D) Heat map of genes that were significantly differentially expressed (T-test q-value<0.05 with FDR correction) depending on the dosage of DYRK1A. Genes are annotated with broad categories of gene function relevant to neural development. (E)-(F), Percentage of EdU labeled cells at 0, 2, or 4 weeks of spontaneous differentiation for DYRK1A knockout (E) or overexpression (F). Values represent mean±SEM from n=3 bioreps. 10,000 cells were analyzed per biorep. (G)-(H), Intensity of MAP2 staining for neurons at 0, 1, 2, 4, or 8 weeks of spontaneous differentiation for DYRK1A knockout (G) or overexpression (F). Values represent mean±SEM from n=2 bioreps with 6 images per biorep. KO, knockout; NT, non-targeting. ****P<0.0001; ***P<0.001; **P<0.01; *P<0.05. ns, not significant.

FIG. 18—Comparison of TF overexpression methods for neuronal differentiation. (A) Schematic of ORF and CRISPR-Cas9 activator comparison. hESCs are transduced with ORF, ORF with UTRs, or SAM CRISPR-Cas9 activator to overexpress NEUROD1 or NEUROG2 for directed differentiation into induced neurons. (B) Expression of NEUROD1 mRNA and protein after NEUROD1 overexpression from n=4 bioreps. (C) Expression of marker genes for neurons (MAP2) and NPs (PAX6) after NEUROD1 overexpression. (D) Expression of NEUROG2 mRNA after NEUROG2 overexpression from n=4 bioreps. (E) Expression of marker genes for neurons (MAP2) and NPs (PAX6) after NEUROG2 overexpression. (F) Intensity of MAP2 staining from n=6 images per condition. All values are mean±SEM. Scale bar, 100 μm. ****P<0.0001; ***P<0.001. ns=not significant. UTR, untranslated region; NT, nontargeting.

FIG. 19—Arrayed TF ORF screen for iNP differentiation. (A) 90 TF ORFs included in the library for the arrayed screen (Table 1). (B) Schematic for arrayed screening (e.g., wells). TF ORFs were individually synthesized, cloned, and packaged into lentivirus for delivery into hESCs. After 4 or 7 days of differentiation, expression of NP marker genes SLC1A3 and VIM were measured to identify candidate TFs. (C) Timeline for arrayed screening. mTeSR stem cell media was incrementally changed to NP media during differentiation, and expression of NP marker genes was measured after 4 and 7 days of differentiation. (D)-(G), Expression of VIM and SLCIA3 mRNA relative to control hESCs overexpressing GFP in NP media from n=3 infection replicates at 4 (D,E) or 7 (F,G) days of differentiation. Candidate TFs (D,F) and other isoforms of candidate TFs (E,G) are indicated.

FIG. 20—A pooled TF ORF screening platform for iNP differentiation. (A) Design of lentiviral vectors for expression of barcoded TFs. WPRE, Woodchuck Hepatitis Virus Posttranscriptional Regulatory Element. (B) Schematic of pooled TF screening with 3 different methods for selecting cell types of interest. For the reporter cell line method, reporter cell lines transduced with the TF library are differentiated and sorted into high or low marker gene-expressing cell populations. For the flow-FISH method, differentiated cells are labeled with FISH probes targeting 2-10 marker genes and sorted based on marker gene expression. For the single-cell RNA sequencing method, differentiated cells can be analyzed using single-cell RNA-seq. In all selection methods, sequencing of TF barcodes enables identification of candidate TFs. (C) FACS plots showing distribution of EGFP expression in SLC1A3 and VIM reporter cell lines with or without the TF library. High and low bins sorted for sequencing of TF barcodes are indicated. (D)-(E), Enrichment of candidate TFs (D) or other isoforms of candidate TFs (D) in the high EGFP-expressing bin relative to the low bin from n=3 infection replicates per reporter cell line. (F) Representative FACS plot showing expression of RPL13A control or SLC1A3 and VIM mRNA labeled by FISH probes from n=3 infection replicates. High and low bins sorted for sequencing of TF barcodes are indicated. (G) Same as (F), showing expression of 10 marker gene mRNA labeled by FISH probes. (H) Comparison of candidate TF enrichment in screens using reporter cell lines and flow-FISH.

FIG. 21—Selection of candidate TFs using single-cell RNA sequencing. (A) Number of cells analyzed using single-cell RNA sequencing (RNA-seq) for each TF isoform out of 59,640 cells. (B) t-distributed stochastic neighbor embedding (tSNE) clustering of single-cell RNA-seq data from hESCs transduced with the TF library. Cells grouped into 18 clusters. (C) Same as (B) highlighting cells expressing a TF of interest. (D) Candidate TFs identified using single-cell RNA-seq. Top, correlations between TF transcriptome signatures and radial glia from human fetal cortex or brain organoid datasets^{20,25,26}. Values represent mean correlation of cells expressing each TF as z-scores. Dashed line indicates cutoff for identifying candidate TFs. Bottom, heat map indicating percentage of cells overexpressing each TF isoform that was grouped into a particular cluster. Candidate TFs selected using single-cell RNA-seq are indicated in blue.

FIG. 22—Validation of candidate TFs for iNP differentiation. (A) Expression of candidate TFs measured using the V5 epitope tag after 7 days of differentiation. (B) Expression of NP marker genes PAX6 and NES in iNPs produced by candidate TFs after 7 days of overexpression. Cell culture media used for each ORF is indicated in parentheses. Scale bar, 50 μm. (C)-(D), Heat map of bulk RNA sequencing (RNA-seq) signature correlation between iNPs and human fetal brain cell types from the Nowakowski 2017 dataset²⁶ (C) or human brain organoids from the Quadrato 2017 dataset²⁵ (D). D7 and D12 indicate whether the ORF was overexpressed for 7 or 12 days, respectively. RG, radial glia; div, dividing; oRG, outer radial glia; tRG, truncated radial glia; vRG, ventricular radial glia; MGE, medial ganglionic eminence; IPC, intermediate progenitor cell; nEN, newborn excitatory neurons, EN, excitatory neurons; PFC, prefrontal cortex; V1, primary visual cortex; nIN, newborn interneurons; IN, interneurons; CTX, cortex; CGE, cortical ganglionic eminence; STR, striatum; OPC, oligodendrocyte precursor cells; Glyc, cells expressing glycolysis genes; Pro, proliferating progenitors; NE, neuroepithelium; DN, dopaminergic neurons; CLN, callosal neurons; CFN, corticofugal neurons; Meso, mesodermal progenitors.

FIG. 23—Characterization of spontaneously differentiated cells produced by candidate TFs in HUES66. Expression of marker genes for neurons (MAP2), astrocytes (GFAP), and oligodendrocyte precursor cells (NG2) after 1, 2, 4, or 8 weeks of spontaneous differentiation for 4 candidate TFs. Scale bar, 100 μm.

FIG. 24—Characterization of iNPs and spontaneously differentiated cells produced by candidate TFs in iPSC11a and H1 pluripotent stem cell lines. (A)-(B), Expression of NP marker genes in iPSC11a iNPs (A) or H1 iNPs (B) after 1 week of TF overexpression. (C)-(D), Expression of marker genes for neurons (MAP2), astrocytes (GFAP), and oligodendrocyte precursor cells (NG2 and PDGFRA) in cells spontaneously differentiated from iPSC11a iNPs (C) or H1 iNPs (D) for 8 weeks. Scale bar, 100 μm.

FIG. 25—Single-cell RNA sequencing profiling of spontaneously differentiated cells produced by candidate TFs. (A) Heat map showing the z-score of the mean log-transformed, normalized counts for each cluster of selected marker genes used to annotate clusters. For a more extensive set of genes, see Table 8. RP, retinal progenitors; RPE, retinal pigment epithelium; RGC, retinal ganglion cells; PR, photoreceptors; DNP, dorsal neural progenitors; RG, radial glia; Astro, astrocytes; CN, cortical neurons; HB&SCN, hindbrain and spinal cord neurons; IN, interneurons; EPD&CPE, ependyma and choroid plexis epithelium; EP, epithelial progenitors; BE, bronchial epithelium; CE, cranial epithelium; NC, neural crest; CNC, cranial neural crest; Pro, uncommitted progenitors; (P), proliferative cells; (S), structural cell types such as bone and cartilage. (B) Distribution of cell types generated in human brain organoids at 6 months from the Quadrato 2017 dataset²⁵.

FIG. 26—ChIP-seq analysis of candidate TFs. (A) Top 3 de novo or known motifs identified using HOMER motif analysis. The names of the TFs with the closest matching motifs, indicating potential cofactors of candidate TFs, are listed. The percentages of ChIP peaks that contained each motif relative to the background, and the associated P-values of enrichment, are also listed. (B)-(C), Example NP marker gene loci with significant ChIP peaks from all 4 candidate TFs for HES1 (B) and BMPR1B (C). (D) Heat map showing percentage of NP-specific TFs or genes that had candidate TF ChIP peaks within 10 kb of the annotated transcriptional start site (TSS). (E) Overlap of NP-specific genes that had candidate TF ChIP peaks within 10 kb of the TSS and were differentially expressed (t-test q-value<0.05 with FDR correction) upon candidate TF overexpression. Blue regions indicate overlap.

FIG. 27—DYRK1A perturbation in RFX4-iNPs to model neurological disorders. (A) Percent indel in RFX4-derived iNPs transduced with DYRK1A KO sgRNAs. Values represent mean±SEM from n=3 bioreps. (B) DYRK1A mRNA expression measured using qPCR probes targeting the endogenous sequence or the codon-optimized ORF sequence. Values represent mean±SEM from n=4 bioreps with 4 technical replicates per biorep. *P<0.05; ND, not detected. (C) Venn diagram showing the number of genes that were significantly differentially expressed (t-test q-value<0.05 with FDR correction) and had an absolute log 2 fold change relative to control that was greater than 1. The KO sgRNAs 1 and 2 conditions were compared to both NT sgRNAs 1 and 2 controls. The ORF condition was compared to GFP control. (D)-(F) Volcano plots showing the number of genes that were significantly differentially expressed (t-test q-value<0.05 with FDR correction) and had an absolute log 2 fold change relative to control that was greater than 1 for DYRK1A KO sgRNA 1 (D), KO sgRNA 2 (E), and ORF (F) conditions. For a full list of genes, see Table 9. (G) Representative images of MAP2 staining during spontaneous differentiation for NT sgRNA 1 and DYRK1A KO sgRNA 2. Scale bar, 100 μm. KO, knockout; NT, non-targeting.

FIG. 28—A barcoded human TF library for directed differentiation. Schematic showing how the TF library can be used to produce differentiated cell types for cellular models and therapies. Puro, puromycin. WPRE, Woodchuck Hepatitis Virus Posttranscriptional Regulatory Element. MOI, multiplicity of infection.

FIG. 29—Development of a multiplexed TF screening platform for directed differentiation. (A) Schematic of multiplexed TF screening. Barcoded TF ORFs are pooled and packaged into lentivirus for delivery into hESCs. TFs that can differentiate hESCs into the cell type of interest are identified using reporter cell line, flow-FISH, or single-cell RNA sequencing (scRNA-seq), followed by deep sequencing of TF barcodes. MOI, multiplicity of infection. (B) Scatterplot showing median enrichment of candidate TFs identified using SLC1A3 or VIM reporter cell lines from n=3 infection replicates. (C) Scatterplot showing average enrichment of candidate TFs identified by flow-FISH with pooled FISH probes targeting 2 or 10 NP marker genes from n=3 infection replicates. (D) Uniform manifold approximation and projection (UMAP) clustering of scRNA-seq data from 53,560 hESCs transduced with the TF library. (E) Heatmap indicating correlations between TF transcriptome signatures and radial glia from human fetal cortex or brain organoid datasets. Values represent mean correlation of cells overexpressing each TF as z-scores. (F) Comparison of TFs that ranked in the top 10% from the 4 different screens.

FIG. 30—Validation of candidate TFs driving iNP differentiation. Top, expression of NP marker genes VIM and NES in iNPs produced by candidate TFs after 7 days of overexpression. Cell culture media used for each ORF is indicated in parentheses. Scale bar, 50 μm. Bottom, heat map of bulk RNA sequencing (RNA-seq) signature correlation between iNPs and human fetal cortex cell types from the Pollen 2015 dataset (Pollen et al., 2015). D7 and D12 indicate the number of days that the ORF was overexpressed. RG, radial glia; IPC, intermediate progenitor cell; N, neuron; IN, interneuron.

FIG. 31—Candidate TFs produce iNPs that can spontaneously differentiate into cell types in the central nervous system. (A) Schematic of spontaneous differentiation. Dox-inducible candidate TFs are transiently overexpressed for 1 week to differentiate hESCs into iNPs, which then spontaneously differentiate for 8 weeks following withdrawal of dox and growth factors. Spontaneously differentiated cells were characterized by immunostaining and single-cell RNA sequencing. rtTA, reverse tetracycline-controlled transactivator; dox, doxycycline; EGF, epidermal growth factor; FGF, fetal growth factor. (B) Expression of marker genes for neurons (MAP2), astrocytes (GFAP), and oligodendrocyte precursor cells (PDGFRA) after 1, 2, 4, or 8 weeks of spontaneous differentiation for 4 candidate TFs. Scale bar, 100 μm.

FIG. 32—Single-cell RNA sequencing of spontaneously differentiated cells from iNPs reveals a broad array of cell types. (A) UMAP clustering of scRNA-seq data from 53,113 cells that have been spontaneously differentiated from iNPs for 8 weeks. iNPs were derived using RFX4, NFIB, ASCL1, or PAX6 with n=2 biological replicates per TF. Colors indicate cell type or state. (B) Data as in (A), with clusters representing central nervous system (CNS) cell types highlighted. Percentage of total cells that contribute to the specified CNS cell type is indicated. (C) Dot plot showing marker genes for each cluster. Circle size indicates percentage of cells expressing the gene in the given cluster and color indicates the mean gene expression value. Horizontal lines distinguish between retinal, CNS, epithelial, and CNC cell types. (D) Cells spontaneously differentiated from each candidate TF are highlighted. Colors indicate biological replicates, S1 and S2. (E) Heatmap showing the percentage of cells from each biological replicate that were grouped into each cluster. (F) Distribution of general cell types produced by each biological replicate. Pro, uncommitted progenitors; RP, retinal progenitors; RPE, retinal pigment epithelium; PR, photoreceptors; RGC, retinal ganglion cells; DNP, dorsal neural progenitors; RG, radial glia; Astro, astrocytes; CN, CNS neurons; EPD, ependyma; EP, epithelial progenitors; BE, bronchial epithelium; CE, cranial epithelium; CNC, cranial neural crest; CNCP, cranial neural crest progenitors; (P), proliferative cells.

FIG. 33—Combining RFX4 with dual SMAD inhibition produces homogenous NPs that generate predominantly GABAergic neurons. (A) UMAP clustering of scRNA-seq data from iNPs derived using different iNP differentiation methods. RFX4-DS-iNPs were produced by combining RFX4 overexpression with dual SMAD inhibition, EB-iNPs were produced using the embryoid body protocol (Schafer et al., 2019), and DS-iNPs were produced using the dual SMAD inhibition protocol (Shi et al., 2012a). Data represents n=2 batch replicates per method with 15,211 RFX4-DS-iNPs, 11,148 EB-iNPs, and 16,421 DS-iNPs. Colors indicate cell type or state. (B) Dot plot showing marker genes for each cluster. Circle size indicates percentage of cells expressing the gene in the given cluster and color indicates the mean expression value. (C) Box plots showing distributions of Euclidean distances between cells within the same batch replicate. Whiskers indicate the 5^{th} and 95^{th} percentiles. (D) Same as (C), for cells between different batch replicates. (E) Data as in (A), highlighting cells derived from each differentiation method. Colors indicate batch replicates, S1 and S2. (F) Heatmap showing the percentage of cells from each batch replicate that were grouped into each cluster. (G) Data as in (A), colored by marker gene expression. (H) UMAP clustering of scRNA-seq data from 26,111 cells that have been spontaneously differentiated from iNPs. iNPs were produced by combining RFX4 overexpression with dual SMAD inhibition and spontaneously differentiated for 4 or 8 weeks. Data represents n=2 biological replicates per timepoint. Colors indicate cell type or state. (I) Dot plot showing marker genes for each cluster. Circle size indicates percentage of cells expressing the gene in the given cluster and color indicates the mean expression value. (J) Data as in (H), colored by marker gene expression. (K) Cells from each time point are highlighted. Colors indicate biological replicates, S1 and S2. (L) Heatmap showing the percentage of cells from each biological replicate that were grouped into each cluster. (M) Distribution of general cell types produced by each biological replicate. NP, neural progenitors; CN, CNS neurons; CNC, cranial neural crest; RG, radial glia; MNG, meninges; P, proliferative cells.

FIG. 34—Modeling neurodevelopmental disorders using RFX4-iNPs with DYRK1A perturbation. (A) Schematic of disease modeling by perturbing DYRK1A expression. Human induced pluripotent stem cells (iPSCs) are transduced with Cas9 and sgRNAs or ORF to knockout or overexpress DYRK1A, respectively. RFX4 is then transiently overexpressed for 1 week to differentiate iPSCs into iNPs, which then spontaneously differentiate for 8 weeks following withdrawal of dox and growth factors. Effects of DYRK1A perturbation were characterized using bulk RNA sequencing, EdU labeling, immunostaining, or electrophysiology. rtTA, reverse tetracycline-controlled transactivator; dox, doxycycline; EGF, epidermal growth factor; FGF, fetal growth factor. (B-D) Volcano plots showing the number of genes that were significantly differentially expressed (t-test q-value<0.05 with FDR correction) and had an absolute log 2 fold change relative to control that was greater than 1 for DYRK1A KO sgRNA 1 (B), KO sgRNA 2 (C), and ORF (D) conditions. For a full list of genes, see Table S3. The KO sgRNAs 1 and 2 conditions were compared to both NT sgRNAs. The ORF condition was compared to GFP control. (E) Venn diagram summarizing the significantly differentially expressed genes in (B-D). (F) Heatmap of genes that were significantly differentially expressed (T-test q-value<0.05 with FDR correction) depending on the dosage of DYRK1A. Genes are annotated with broad categories of gene function relevant to neural development. Average gene expression measurements across n=3 biological replicates are shown. (G-H) Percentage of EdU labeled cells at 0, 2, or 4 weeks of spontaneous differentiation for DYRK1A knockout (G) or overexpression (H). Values represent mean±SEM from n=3 biological replicates. 10,000 cells were analyzed per biological replicate. (I-J) Intensity of MAP2 staining for neurons at 0, 1, 2, 4, or 8 weeks of spontaneous differentiation for DYRK1A knockout (I) or overexpression (J). Values represent mean±SEM from n=2 biological replicates with 6 images per biological replicate. KO, knockout; NT, non-targeting. ****P<0.0001; ***P<0.001; **P<0.01; *P<0.05. ns, not significant.

FIG. 35—Comparison of TF overexpression methods for neuronal differentiation. (A) Schematic of ORF and CRISPR-Cas9 activator comparison. hESCs are transduced with ORF, ORF with UTRs, or SAM CRISPR-Cas9 activator to overexpress NEUROD1 or NEUROG2 for directed differentiation into induced neurons. (B) Expression of NEUROD1 mRNA and protein after NEUROD1 overexpression from n=4 biological replicates. (C) Expression of marker genes for neurons (MAP2) and NPs (PAX6) after NEUROD1 overexpression. (D) Expression of NEUROG2 mRNA after NEUROG2 overexpression from n=4 biological replicates. (E) Expression of marker genes for neurons (MAP2) and NPs (PAX6) after NEUROG2 overexpression. (F) Intensity of MAP2 staining from n=6 images per condition. All values are mean±SEM. Scale bar, 100 μm. ****P<0.0001; ***P<0.001. ns=not significant. UTR, untranslated region; NT, nontargeting.

FIG. 36—A multiplexed TF ORF screening platform for iNP differentiation. (A) Timeline for screening. mTeSR stem cell media was incrementally changed to NP media during differentiation, and cells were harvested after 7 days of differentiation. (B) FACS histograms showing distribution of EGFP expression in SLC1A3 and VIM reporter cell lines with or without the TF library. High and low bins sorted for sequencing of TF barcodes are indicated. (C) Scatterplot showing enrichment of alternative isoforms of candidate TFs identified using SLC1A3 or VIM reporter cell lines from n=3 infection replicates. (D) Representative FACS plot showing expression of RPL13A control or SLC1A3 and VIM mRNA labeled by FISH probes from n=3 infection replicates. High and low bins sorted for sequencing of TF barcodes are indicated. (E) Same as (D), showing expression of 10 marker gene mRNA labeled by FISH probes. (F) Scatterplot showing enrichment of alternative isoforms of candidate TFs identified by flow-FISH with pooled FISH probes targeting 2 or 10 NP marker genes from n=3 infection replicates. (G) Comparison of candidate TF enrichment in screens using reporter cell lines and flow-FISH. (H) Number of cells analyzed using single-cell RNA sequencing (RNA-seq) that were assigned to each TF isoform out of 53,560 cells. (I) Uniform manifold approximation and projection (UMAP) clustering of single-cell RNA-seq data from hESCs transduced with the TF library. Cells expressing TFs of interest are highlighted. (J) Z-score of median Euclidean distances between cells expressing a TF and the rest of the cells. Distances were calculated using 939 highly variable genes. (K) Heatmap showing relative marker gene expression of cell types from the mouse organogenesis cell atlas (Cao Nature 2019) in cells overexpressing each TF isoform. The top 30 marker genes for each cell type were used to determine marker gene enrichment as z-scores. Candidate TFs selected using single-cell RNA-seq are indicated in blue.

FIG. 37—Validation of candidate TFs identified by pooled screens for iNP differentiation. (A) Schematic for arrayed screening. TF ORFs were individually synthesized, cloned, and packaged into lentivirus for delivery into hESCs. After 7 days of differentiation, expression of NP marker genes SLC1A3 and VIM was measured to identify candidate TFs. (B-C) Expression of VIM and SLC1A3 mRNA relative to control hESCs overexpressing GFP in NP media from n=3 infection replicates. Candidate TFs (B) and alternative isoforms of candidate TFs (C) are indicated. (D) Western blot showing expression of candidate TFs measured using the V5 epitope tag after 7 days of differentiation. (E) Top, expression of NP marker genes PAX6 and NES in iNPs produced by candidate TFs after 7 days of overexpression. Cell culture media used for each ORF is indicated in parentheses. Scale bar, 50 μm. Middle and bottom, Heatmaps of bulk RNA sequencing (RNA-seq) signature correlation between iNPs and human fetal brain cell types from the Nowakowski 2017 dataset (middle) or human brain organoids from the Quadrato 2017 dataset (bottom). D7 and D12 indicate whether the ORF was overexpressed for 7 or 12 days, respectively. RG, radial glia; div, dividing; oRG, outer radial glia; tRG, truncated radial glia; vRG, ventricular radial glia; MGE, medial ganglionic eminence; IPC, intermediate progenitor cell; nEN, newborn excitatory neurons, EN, excitatory neurons; PFC, prefrontal cortex; V1, primary visual cortex; nIN, newborn interneurons; IN, interneurons; CTX, cortex; CGE, cortical ganglionic eminence; STR, striatum; OPC, oligodendrocyte precursor cells; Glyc, cells expressing glycolysis genes; Pro, proliferating progenitors; NE, neuroepithelium; DN, dopaminergic neurons; CLN, callosal neurons; CFN, corticofugal neurons; Meso, mesodermal progenitors.

FIG. 38—Characterization of iNPs and spontaneously differentiated cells produced by candidate TFs in different stem cell lines. (A) Expression of marker genes for neurons (MAP2), astrocytes (GFAP), and oligodendrocyte precursor cells (NG2) in cells spontaneously differentiated for 1, 2, 4, or 8 weeks from HUES66 iNPs produced by 4 candidate TFs. (B-C) Expression of NP marker genes in iPSC11a iNPs (B) or H1 iNPs (C) after 1 week of TF overexpression. (D-E) Expression of marker genes for neurons (MAP2), astrocytes (GFAP), and oligodendrocyte precursor cells (NG2 and PDGFRA) in cells spontaneously differentiated from iPSC11a iNPs (D) or H1 iNPs (E) for 8 weeks. Scale bar, 100 μm.

FIG. 39—Profiling spontaneously differentiated neurons from iNPs by single-cell RNA sequencing and target genes of candidate TFs by ChIP-seq. (A-E) UMAP clustering of single-cell RNA-seq data from 4,162 neurons that have been spontaneously differentiated from iNPs for 8 weeks. iNPs were derived using RFX4, NFIB, ASCL1, or PAX6 with n=2 biological replicates per TF. (A-D) or biological replicates (E). (A-D) Marker genes for general regions of the central nervous systems (A), newborn cortical excitatory neurons (B), neuronal subtypes (C), and cortical projection neurons (D) are shown. Colors indicate gene expression. (E) Neurons spontaneously differentiated from each candidate TF are highlighted. Colors indicate biological replicates, S1 and S2. (F) Top 3 de novo or known motifs identified using HOMER motif analysis. The names of the TFs with the closest matching motifs, indicating potential cofactors of candidate TFs, and the associated P-values of enrichment are listed. G, Heatmap showing percentage of NP-specific TFs or genes that had candidate TF ChIP peaks within 10 kb of the annotated transcriptional start site (TSS). (H-I) Overlap of NP-specific genes that had candidate TF ChIP peaks within 10 kb of the TSS and were differentially expressed (t-test q-value<0.05 with FDR correction) upon candidate TF overexpression. Genes that were shared between candidate TFs are shown in (H), with blue regions indicating overlap, and genes unique to each candidate TF are shown in (I).

FIG. 40—Characterization of iNPs produced by combining RFX4 with dual SMAD inhibition. (A) Schematic for different media conditions (M1-M8) tested. SMAD inhibitors dorsomorphin (DM) and SB-431542 (SB) were added to the media at the indicated concentrations. mTeSR stem cell media was changed to different NP media (NP, EB, and DS; see Methods) over 7 days of differentiation. (B) Heatmaps showing expression of neuron marker genes TUJ1 and MAP2 relative to GAPDH control in cells from iNPs that have undergone spontaneous neurogenesis for 2 or 4 weeks. iNPs were differentiated for 5 or 7 days using each of the media conditions in (A) and seeded at low or high densities prior to spontaneous neurogenesis. Colors represent mean expression from n=4 biological replicates. (C) Same as (A), for additional media conditions tested. (D) Same as (B), for the media conditions shown in (C). (E) UMAP clustering of scRNA-seq data from iNPs derived using different iNP differentiation methods. Marker genes for the telencephalon are shown. Data represents n=2 batch replicates per method with 15,211 RFX4-DS-iNPs, 11,148 EB-iNPs, and 16,421 DS-iNPs. Colors indicate gene expression. (F) Expression of NP marker genes NES and FOXG1 in iNPs produced by different NP differentiation methods. RFX4-DS-iNPs were produced by combining RFX4 overexpression with dual SMAD inhibition, EB-iNPs were produced using the embryoid body protocol (Schafer et al., 2019), and DS-iNPs were produced using the dual SMAD inhibition protocol (Shi et al., 2012a). Scale bar, 50 μm. (G-J) UMAP clustering of single-cell RNA-seq data from 26,111 cells that have been spontaneously differentiated from iNPs. iNPs were produced by combining RFX4 overexpression with dual SMAD inhibition and spontaneously differentiated for 4 or 8 weeks. Data represents n=2 biological replicates per timepoint. Marker genes for general regions of the central nervous systems (G), radial glia subtypes (H), neuronal subtypes (I), and GABAergic interneuron subtypes (J) are shown. Colors indicate gene expression.

FIG. 41—Perturbations of DYRK1A in RFX4-iNPs for modeling neurological disorders. (A) Percent indels in RFX4-iNPs transduced with DYRK1A KO sgRNAs. Values represent mean±SEM from n=3 biological replicates. (B) DYRK1A mRNA expression measured using qPCR probes targeting the endogenous sequence or the codon-optimized ORF sequence. Values represent mean±SEM from n=4 biological replicates with 4 technical replicates per biological replicate. ND, not detected. (C-D) Western blot of DYRK1A at 7 days after transduction with Cas9 and DYRK1A KO sgRNAs (C) or DYRK1A ORF (D). (E) Representative images of MAP2 staining during spontaneous differentiation for NT sgRNA 1 and DYRK1A KO sgRNA 2. Scale bar, 100 μm. (F) Representative electrophysiology traces for neurons with or without evoked action potentials (AP) and spontaneous excitatory postsynaptic currents (EPSCs). (G) Proportion of neurons with or without AP and EPSCs for different DYRK1A perturbations from n=31-45 neurons. (H-I) Intrinsic membrane (H) and action potential (I) properties measured using electrophysiology for different DYRK1A perturbations from n=12-36 neurons with evoked action potentials. Mean±SEM indicated on graph. *P<0.05.

FIG. 42—Building a TF Atlas of directed differentiation. (A) Schematic of TF Atlas setup. All 3,550 barcoded TF ORFs from the MORF library were packaged into lentivirus for delivery into human embryonic stem cells (hESCs) at a low multiplicity of infection (MOI). After 7 days of TF ORF overexpression, cells were profiled using single-cell RNA sequencing (scRNA-seq) to map TF ORFs to expression changes. (B-D) Uniform manifold approximation and projection (UMAP) of scRNA-seq data from 671,453 cells overexpressing 3,266 TF isoforms. Colors indicate Louvain clusters (B), gene expression (C), and diffusion pseudotime (D). (E) Smoothened heat map of the top 1,000 upregulated and downregulated genes over diffusion pseudotime. Gene expression in each row is represented as z-scores. Genes are ordered based on the slope of expression change over pseudotime fitted using linear regression. (F-G) Most enriched pathways among the top 100 upregulated (F) and downregulated (G) genes. (H) Heat map showing significance of the difference between assigned pseudotimes of cells expressing each TF isoform and those expressing controls. TF isoforms are grouped by gene. Only 320 TF genes with multiple isoforms, at least one of which induces a significantly different pseudotime than control, are included.

FIG. 43—Unbiased grouping of TFs based on gene programs. (A) Heat maps showing pairwise Pearson correlation (top) and enrichment of 100 gene programs (bottom) identified using non-negative matrix factorization (NMF) on mean expression profiles of 3,266 TF ORFs. TFs are ordered by hierarchical clustering. Each TF ORF is annotated by TF family and average diffusion pseudotime relative to control. Some TF groups are labeled and annotated based on known relationships. Numbers in parentheses indicate the number of TF isoforms that were found in the same group. (B-C) Zoomed in subsets of (A) with top enriched pathway annotated for each gene program. (D) UMAP of scRNA-seq data highlighting enrichment of each gene program.

FIG. 44—Mapping TF ORFs in differentiated cells to reference cell types. (A-B) UMAP of scRNA-seq data from 28,825 differentiated cells. Cells from clusters 6-8 of the TF Atlas shown in FIG. 42B were reclustered for further characterization. Colors indicate Louvain clusters (A) and nominated cell type from the human fetal cell atlas (Cao Science 2020) (B). Cell type matches with score >0.3 are highlighted. (C-D) Heat maps showing percentage of cells with the indicated TF ORF that were assigned to each cluster (C) or nominated cell type (D). Numbers after TF gene names indicate the isoform. Percentages are determined by normalizing to the total number of cells overexpressing the indicated TF in the entire TF Atlas. Only the 5 most enriched TF ORFs that are greater than 5% are shown. EMT, epithelial-mesenchymal transition; ENS, enteric nervous system.

FIG. 45—Validation of candidate TFs for differentiation towards nominated cell types. (A) Expression of marker genes for each nominated cell type in H1 hESCs after 7 days of candidate TF or GFP overexpression. Numbers after TF gene names indicate the isoform. n=4. (B-C) Scatterplot comparing expression of 205 marker genes in H1 hESCs to H9 hESCs (B) or 11a iPSCs (C). Expression is measured as average fold change in cells overexpressing candidate TF relative to GFP. (D-K) Left, expression of marker genes in H1 hESCs after 7 days of candidate TF overexpression. Right, intensity of marker gene staining from n=6 images per condition. Mean intensity per cell is normalized to cells overexpressing the GFP control. Scale bar, 25 μm. Marker genes for neuron (D), EMT smooth muscle (E), endothelial (F), smooth muscle (G), metanephric (H), intestinal epithelial (I), lung ciliated epithelial (J), and trophoblast (K) cells are shown. EMT, epithelial-mesenchymal transition. Values represent mean±SEM. ****P<0.0001; ***P<0.001; **P<0.01; *P<0.05.

FIG. 46—Targeted TF overexpression screening platform for directed differentiation. (A) Schematic of targeted TF screening. A subset of TFs are pooled from the MORF library and packaged into lentivirus for delivery into hESCs. TFs that can differentiate hESCs into the cell type of interest are identified using reporter cell line, flow-FISH, or scRNA-seq, followed by deep sequencing of TF barcodes. MOI, multiplicity of infection. (B) Comparison of TFs that ranked in the top 10% from the 4 different screens for induced neural progenitor (iNP) differentiation. (C) Expression of markers for neurons (MAP2), astrocytes (GFAP), and oligodendrocyte precursor cells (PDGFRA) after 1, 2, 4, or 8 weeks of spontaneous differentiation from RFX4-iNPs. Scale bar, 100 μm. (D-F) ScRNA-seq data from 26,111 cells that have been spontaneously differentiated from iNPs for 4 or 8 weeks. iNPs were produced by RFX4-DS-iNPs. Data represents n=2 biological replicates per timepoint. NP, neural progenitors; CN, CNS neurons; CNC, cranial neural crest; RG, radial glia; MNG, meninges; (P), proliferative cells. (D) UMAP clustering results with colors indicating Louvain clusters. (E) Dot plot showing marker genes for each cluster. Circle size indicates percentage of cells expressing the gene in the given cluster and color indicates the mean expression value. (F) Distribution of general cell types produced by each biological replicate. (G-J) Disease modeling by knocking out or overexpressing DYRK1A in human induced pluripotent stem cells (iPSCs) and differentiating into neural progenitors using RFX4. (G-H) Percentage of EdU labeled cells at 0, 2, or 4 weeks of spontaneous differentiation for DYRK1A knockout (G) or overexpression (H). n=3 biological replicates. (I-J) Intensity of MAP2 staining for neurons at 0, 1, 2, 4, or 8 weeks of spontaneous differentiation for DYRK1A knockout (I) or overexpression (J). n=12 images. Values represent mean±SEM. KO, knockout; NT, non-targeting; sg, single guide RNA. ****P<0.0001; ***P<0.001; **P<0.01; *P<0.05; ns, not significant.

FIG. 47—Regulatory networks by joint profiling of chromatin accessibility and gene expression under TF overexpression. (A) Weighted nearest neighbor (WNN) UMAP of joint chromatin accessibility and gene expression measured by scATAC- and scRNA-seq, respectively, from 69,085 cells overexpressing 198 TF isoforms for 4 or 7 days. Colors indicate clusters identified by the smart local moving (SLM) algorithm. (B) Dot plot showing marker genes for each cluster. Color indicates the expression and circle size indicates chromatin accessibility. Values represent average fold change relative to other clusters. (C-E) Example marker gene chromatin accessibility (left) and expression (right) for different clusters compared to the undifferentiated cluster 0. Genes that show strong (C), weak (D), and no (E) correlation between ATAC and RNA profiles are included. (F) Heat maps showing the top TF ORF (left) and nominated regulators (right) for each cluster. Left, percentage of cells with the indicated TF ORF is shown. Numbers after TF gene names indicate the isoform. Percentages are determined by normalizing to the total number of cells with the TF ORF in the joint scATAC- and scRNA-seq dataset. Only the 6 most enriched TF ORFs that are greater than 5% are shown. Right, average AUC (area under the ROC curve) of TF motif enrichment and RNA expression is shown. TFs with significantly enriched (FDR<0.05) motif and expression in each cluster are included. TFs that were identified as top ORFs and regulators are labeled in blue.

FIG. 48—Combinatorial TF screening and prediction. (A) UMAP of scRNA-seq profiles from the combinatorial TF screen in hESCs. Each circle represents the mean expression profile of cells overexpressing the indicated TF ORF(s). The screen included 10 TF ORFs in combinations, including 44 doubles and 3 triples, as well as 10 singles. Example single TF profiles with associated grouping of TF combinations (CDX1, FLI1, and KLF4) are indicated with black borders. (B-C) Percent accuracy for different approaches to predict TFs for measured double (B) or triple (C) TF expression profiles. Single TF profiles were averaged or fitted with linear regression models against double or triple TF profiles. Combinations of single TF profiles were ranked by similarity to the measured combinatorial TF profile. The nominated combinations were compared to the known TF combinations of the measured combinatorial TF profiles to assess accuracy. Kernel ridge and random forest regression algorithms did not significantly outperform random selection for triplet prediction and were excluded. (D-I) Cell type prediction results for double TF profiles. Known combinations (D) or predicted combinations for hepatoblasts (E), bronchiolar and alveolar epithelial cells (F), metanephric cells (G), vascular endothelial cells (H), and trophoblast giant cells (I) are shown. TF combinations were ranked by the gene signature scores for each respective cell type. As gene signature scores were discrete, the percentile ranks were reported as ranges. For predicted combinations, TFs that are part of known combinations, developmentally critical, or specifically expressed in the target cell types are indicated in blue.

FIG. 49—Comparison of TF overexpression methods for neuronal differentiation. (A) Schematic of ORF and CRISPR activator (CRISPRa) comparison. hESCs are transduced with ORF, ORF with UTRs, or SAM CRISPRa to upregulating NEUROD1 or NEUROG2 for directed differentiation into induced neurons. (B) Expression of NEUROD1 mRNA and protein after NEUROD1 upregulation. n=4. (C) Expression of marker genes for neurons (MAP2) and neural progenitors (PAX6) after NEUROD1 upregulation. (D) Expression of NEUROG2 mRNA after NEUROG2 upregulation. n=4. (E) Expression of marker genes for neurons (MAP2) and NPs (PAX6) after NEUROG2 upregulation. (F) Intensity of MAP2 staining normalized to nuclei count. n=6. All values are mean±SEM. Scale bar, 100 μm. ****P<0.0001; ***P<0.001; ns, not significant. UTR, untranslated region; NT, nontargeting; sg, single guide RNA.

FIG. 50—Bulk TF screening in different cell culture media. (A) Design of barcoded TF ORF lentiviral vectors. WPRE, Woodchuck Hepatitis Virus Posttranscriptional Regulatory Element. (B) Schematic of bulk TF screening. All 3,550 barcoded TF ORFs from the MORF library were packaged into lentivirus for delivery into hESCs at a low multiplicity of infection (MOI). After 7 days of TF ORF overexpression in 7 different cell culture media, cells were stained for stem cell markers (TRA-1-60 and SSEA4) and sorted to enrich for stem and differentiated cells. Deep sequencing of TF barcodes profiled changes in TF distribution. (C) Scatterplots comparing the TF barcode distribution for the initial plasmid and lentiviral libraries to the unsorted cells cultured in 7 different medias (M1-M7, see methods) after 7 days of TF ORF overexpression. BR1 and BR2 indicate the two biological replicates. Skew represents the ratio between the 90^{th} and 10^{th} percentile barcode counts. (D) Heat map showing the fold change in TF barcodes in each media condition relative to the initial lentivirus library. The top 10 most enriched and depleted TF barcodes are labeled. Numbers after the TF gene name indicate the isoform. (E) Heat map showing pairwise Pearson correlation between each of the conditions in (D). Conditions are ordered by hierarchical clustering. (F-G) Scatterplots showing the relationship between TF barcode counts and ORF length for the lentivirus library (F) and the average unsorted cells after 7 days of overexpression (G).

FIG. 51—Bulk TF screening to evaluate effects of media on TF-induced differentiation outcome. (A) Scatterplots showing the fold change in TF barcodes in the sorted differentiated cells relative to stem cells for each media condition (M1-M7, see methods). BR1 and BR2 indicate the two biological replicates. TFs with known roles in development or differentiation are labeled. (B) Heat map summarizing the fold changes in (A) for each TF isoform. The top 50 most enriched TFs are labeled. Numbers after the TF gene name indicate the isoform. (C) Data as in (B), highlighting the TFs with known roles in development or differentiation. (D) Heat map showing the pairwise Pearson correlation between each of the conditions in (B). The top 5% of TFs with the highest average fold change were evaluated. Conditions are ordered by hierarchical clustering. (E) Box plots showing fold enrichment of 67 developmentally critical TFs (Parekh Cell Systems 2018 and this study) for each media condition. Whiskers indicate the 10th and 90th percentiles.

FIG. 52—Data quality control for the TF Atlas. (A) Violin plots showing distribution of genes, unique molecular identifiers (UMIs), and percent mitochondrial counts per cell in the TF Atlas. (B) Comparison of TF ORF distributions between the bulk TF screen and the TF Atlas scRNA-seq. For each TF ORF, barcode counts per million (CPM) from the bulk screen is compared to the number of cells per TF in the TF Atlas. (C) Distribution of cells overexpressing each TF isoform. Cells were subsampled or filtered by TF ORF such that each TF had between 3 and 1,000 cells in the TF Atlas. (D) Scatterplot showing the relationship between average expression of the TF ORF per cell to the TF ORF length. (E) Density scatterplot showing, for each cell, expression of the TF ORF and the corresponding endogenous TF. TF ORF expression is measured using barcode counts and endogenous TF expression is measured using scRNA-seq counts. (F) UMAP of TF Atlas scRNA-seq data highlighting cells with indicated ORF. Numbers after TF gene names indicate the isoform. (G) Heat maps showing percentage of cells with the indicated TF ORF that were assigned to each cluster.

FIG. 53—Pseudotime analysis for ordering cells in differentiation trajectories. (A-B) Force-directed graph (FDG) representation of TF Atlas scRNA-seq data. Colors indicate Louvain clusters (A) and diffusion pseudotime (B). (C) Stream plot of velocities shown on the UMAP of TF Atlas scRNA-seq data from 671,453 cells overexpressing 3,266 TF isoforms. Colors indicate Louvain clusters. (D) UMAP of TF Atlas scRNA-seq data. Colors indicate RNA velocity pseudotimes. (E) FDG representation of (C). (F) FDG representation of (D). (G) Density scatterplots comparing the diffusion pseudotimes to RNA velocity for each cell. (H-J) Density scatterplots showing the number of genes (H), UMIs (I), and TF barcode counts (J) over diffusion pseudotime for each cell. (K) Comparison of the average euclidean distance and pseudotime for cells overexpressing TFs relative to those overexpressing controls.

FIG. 54—Differentially expressed genes across pseudotime. (A) Smoothened heat map of the top 1,000 upregulated and downregulated genes over RNA velocity. Gene expression in each row is represented as z-scores. Genes are ordered based on the slope of expression change over pseudotime fitted using linear regression. (B) Gene expression along trajectories calculated with diffusion (left) or RNA velocity (right). (C) Scatterplot comparing the differentiation results of the scRNA-seq pseudotime analysis to the bulk TF screen. For the scRNA-seq screen, the average pseudotime of cells overexpressing TFs relative to those overexpressing GFP or mCherry controls is shown. For the bulk TF screen, the average fold change in the corresponding TF barcodes in the sorted differentiated cells relative to stem cells is shown. (D) Significance of the difference between assigned pseudotimes of cells expressing each TF isoform and those expressing controls. Subset of TF isoforms from FIG. 42H are included. Dashed line indicates the threshold above which FDR<0.05.

FIG. 55—Unbiased clustering of TFs based on Pearson correlation of gene expression. (A) Heat map showing pairwise Pearson correlation for mean expression profiles of 3,266 TF ORFs. TFs are ordered by hierarchical clustering. Each TF is annotated by TF family and average pseudotime relative to control. Some TF groups are labeled and annotated based on known relationship. (B-C) Zoomed in subsets of (A).

FIG. 56—Differential gene expression analysis and cell type mapping for differentiated cells. (A) Smoothened heat map showing expression of marker genes for each cluster of differentiated cells from FIG. 44A. Cells are sorted by cluster followed by diffusion pseudotime. Gene expression in each column is represented as z-scores. (B) Heat map showing percentage of cells from each cluster that mapped to the indicated reference cell type. EMT, epithelial-mesenchymal transition; ENS, enteric nervous system. (C) Heat map showing enrichment of Gene Ontology (GO) biological process terms in differentially expressed genes for each cluster. CNS, central nervous system; diff., differentiation; reg., regulation; dev., development; migr., migration.

FIG. 57—Expression of marker genes across stem cell lines and in additional nominated cell types. (A) Heat map showing expression of marker genes in H1 hESCs (left), H9 hESCs (middle), or 11a iPSCs (right) after 7 days of candidate TF or GFP overexpression. Expression is shown as average fold change in cells overexpressing candidate TF relative to GFP. Numbers after TF gene names indicate the isoform. (B) Expression of marker genes for each nominated cell type in H1 hESCs after 7 days of candidate TF or GFP overexpression. n=4. Values represent mean±SEM. ****P<0.0001; ***P<0.001; **P<0.01; *P<0.05; ns, not significant.

FIG. 58—Validation of candidate TFs in other stem cell lines for differentiation towards nominated cell types. (A-B) Expression of marker genes for each nominated cell type in H9 hESCs (A) or 11a iPSCs (B) after 7 days of candidate TF or GFP overexpression. Numbers after TF gene names indicate the isoform. n=4. Values represent mean±SEM. ****P<0.0001; ***P<0.001; **P<0.01; *P<0.05; ns, not significant; ND, not detected.

FIG. 59—Immunostaining of marker genes to validate candidate TFs for inducing differentiation of nominated cell types. (A-C) Left, expression of marker genes in H1 hESCs after 7 days of candidate TF or GFP overexpression. Right, intensity of marker gene staining from n=6 images per condition. Numbers after TF gene names indicate the isoform. Mean intensity per cell is normalized to cells overexpressing the GFP control. Scale bar, 25 μm. Marker genes for stromal (A), intestinal epithelial (B), and lung ciliated epithelial (C) cells are shown. Values represent mean±SEM. **P<0.01; *P<0.05; ns, not significant. (D) Expression of marker genes in H1 hESCs after 7 days of GFP overexpression. Controls for data in FIG. 45D-K.

FIG. 60—A targeted TF ORF screening platform for iNP differentiation. (A) Timeline for screening. mTeSR stem cell media was incrementally changed to neural progenitor media during differentiation, and cells were harvested after 7 days of differentiation. (B) FACS histograms showing distribution of EGFP expression in SLC1A3 and VIM reporter cell lines with or without the TF library. High and low bins sorted for sequencing of TF barcodes are indicated. (C-D) Scatterplots showing enrichment of candidate TFs (C) and alternative isoforms (D) identified using SLC1A3 or VIM reporter cell lines. n=3 replicates per reporter cell line. (E-F) Representative FACS plots showing expression of 2 (E) or 10 (F) NP marker genes labeled by pooled FISH probes. High and low bins sorted for sequencing of TF barcodes are indicated. (G-H) Scatterplot showing enrichment of candidate TFs (G) and alternative isoforms (H) identified by flow-FISH with pooled FISH probes targeting 2 or 10 NP marker genes. n=3 replicates per flow-FISH screen. (I) Comparison of candidate TF enrichment in screens using reporter cell lines and flow-FISH.

FIG. 61—TF ORF screening with single-cell RNA-sequencing and in an arrayed format. (A-G) TF ORF screening using single-cell RNA sequencing (scRNA-seq) on 60,997 cells as readout. (A) Violin plots showing distribution of genes, unique molecular identifiers (UMIs), and percent mitochondrial counts per cell. (B) Distribution of cells overexpressing each TF isoform. (C) Comparison of TF ORF expression per cell measured by TF barcode counts and TF ORF length. Data represents mean±SEM. (D-E) Uniform manifold approximation and projection (UMAP) clustering of scRNA-seq data. Colors indicate Louvain clusters (D) or cells expressing TFs of interest (E). (F) Z-score of mean Euclidean distances between cells expressing a TF and the rest of the cells. (G) Heatmap indicating correlations between mean expression profiles of cells overexpressing each TF and human radial glia from published datasets (14, 22-25). Values represent z-scores of Pearson correlation. (H-I) Scatterplots showing enrichment of candidate TFs (H) and alternative isoforms (I) identified using arrayed screening format. TF ORFs were individually packaged into lentivirus for delivery into hESCs. Expression of marker genes SLC1A3 and VIM was measured to identify candidate TFs. N=3 screening replicates.

FIG. 62—Validation of candidate TFs driving iNP differentiation. (A) Western blot showing expression of candidate TFs measured using the V5 epitope tag after 7 days of differentiation. (B) Top, expression of NP markers VIM and NES in iNPs produced by candidate TFs after 7 days of overexpression. Cell culture media used for each ORF is indicated in parentheses. Scale bar, 50 μm. Bottom, heat maps showing correlation between expression profiles of iNPs and human fetal cortex or brain organoid cell types from 3 datasets (14, 23, 24). D7 and D12 indicate the number of days that the ORF was overexpressed. RG, radial glia; IPC, intermediate progenitor cell; N, neuron; IN, interneuron; div, dividing; oRG, outer radial glia; tRG, truncated radial glia; vRG, ventricular radial glia; MGE, medial ganglionic eminence; nEN, newborn excitatory neurons, EN, excitatory neurons; PFC, prefrontal cortex; V1, primary visual cortex; nIN, newborn interneurons; CTX, cortex; CGE, cortical ganglionic eminence; STR, striatum; OPC, oligodendrocyte precursor cells; Glyc, cells expressing glycolysis genes; Pro, proliferating progenitors; NE, neuroepithelium; DN, dopaminergic neurons; CLN, callosal neurons; CFN, corticofugal neurons; Meso, mesodermal progenitors.

FIG. 63—Characterization of cells spontaneously differentiated from iNPs generated by candidate TFs. (A) Schematic of spontaneous differentiation. Dox-inducible candidate TFs are transiently overexpressed for 1 week to differentiate hESCs into iNPs, which then spontaneously differentiate for 8 weeks following withdrawal of dox and growth factors. Spontaneously differentiated cells were characterized by immunostaining and single-cell RNA sequencing. dox, doxycycline; EGF, epidermal growth factor; FGF, fetal growth factor. (B-C) Expression of marker genes for neurons (MAP2), astrocytes (GFAP), and oligodendrocyte precursor cells [PDGFRA (B) or NG2 (C)] in cells spontaneously differentiated for 1, 2, 4, or 8 weeks from iNPs produced by candidate TFs. Scale bar, 100 μm.

FIG. 64—Validation of candidate TFs in other stem cell lines for iNP differentiation. (A-B) Expression of NP marker genes in iNPs generated using 11a iPSC (A) or H1 hESC (B) lines after 1 week of TF overexpression. (C-D) Expression of marker genes for neurons (MAP2), astrocytes (GFAP), and oligodendrocyte precursor cells (NG2 and PDGFRA) in cells spontaneously differentiated from 11a iPSC iNPs (C) or H1 hESC iNPs (D) for 8 weeks. Scale bar, 100 μm.

FIG. 65—Differentiation of cardiomyocytes from EOMES-derived progenitors. (A) Percent of cells that stained for TNNT2 at day 10 for different EOMES induction times and seeding densities pooled from n=3 biological replicates. (B) Percent of cells that stained for TNNT2 at day 10 after 2 days of EOMES induction or GSK and Wnt inhibition pooled from n=3 biological replicates. (C) Expression of cardiomyocyte markers TNNT2 and NKX2.5 at day 30 after 2 days of EOMES induction or GSK and Wnt inhibition. Scale bar, 100 μm. (D) UMAP clustering of single-cell RNA-seq data from 16,698 cells that have been spontaneously differentiated for 4 weeks after EOMES induction or GSK and Wnt inhibition. Data represents n=2 biological replicates per differentiation method. Colors indicate cell types of state. (E) Dot plot showing marker genes for each cluster. Circle size indicates percentage of cells expressing the gene in the given cluster and color indicates the mean expression value. (F) Data as in (D), colored by marker gene expression. (G) Heatmap showing the percentage of cells from each biological replicate that were grouped into each cluster. (H) Distribution of general cell types produced by each biological replicate. (I) Cells derived using each differentiation method are highlighted. Colors indicate biological replicates, S1 and S2. (J) Data as in (D), highlighting expression of marker genes for mature cardiomyocytes. (K) Violin plots showing expression of marker genes for mature cardiomyocytes for each biological replicate. VCM, ventricular cardiomyocytes; ACM, atrial cardiomyocytes; SMM, smooth muscle cells; SKM, skeletal muscle cells; EPTH, epithelial cells; (P), proliferative cells.

FIG. 66—Profiling cells spontaneously differentiated from iNPs using single-cell RNA sequencing. (A) UMAP clustering of scRNA-seq data from 53,113 cells that have been spontaneously differentiated from iNPs for 8 weeks. iNPs were derived using RFX4, NFIB, ASCL1, or PAX6 with n=2 biological replicates per TF. Colors indicate Louvain clusters. (B) Dot plot showing marker genes for each cluster. Circle size indicates percentage of cells expressing the gene in the given cluster and color indicates the mean expression value. Horizontal lines distinguish between major cell types. Pro, uncommitted progenitors; RP, retinal progenitors; RPE, retinal pigment epithelium; PR, photoreceptors; RGC, retinal ganglion cells; DNP, dorsal neural progenitors; RG, radial glia; Astro, astrocytes; CN, CNS neurons; EPD, ependyma; EP, epithelial progenitors; BE, bronchial epithelium; CE, cranial epithelium; CNC, cranial neural crest; CNCP, cranial neural crest progenitors; (P), proliferative cells.

FIG. 67—Single-cell RNA sequencing comparison of spontaneously differentiated cells produced by candidate TF iNPs. (A-B) UMAP clustering of scRNA-seq data from 53,113 cells that have been spontaneously differentiated from iNPs for 8 weeks. iNPs were derived using RFX4, NFIB, ASCL1, or PAX6 with n=2 biological replicates per TF. (A) Clusters representing central nervous system (CNS) cell types highlighted. Percentage of cells that contribute to the specified CNS cell type is indicated. (B) Cells spontaneously differentiated from each candidate TF are highlighted. Colors indicate biological replicates, S1 and S2. (C) Heatmap showing the percentage of cells from each replicate that were grouped into each cluster. (D) Distribution of general cell types produced by each biological replicate. Pro, uncommitted progenitors; RP, retinal progenitors; RPE, retinal pigment epithelium; PR, photoreceptors; RGC, retinal ganglion cells; DNP, dorsal neural progenitors; RG, radial glia; Astro, astrocytes; CN, CNS neurons; EPD, ependyma; EP, epithelial progenitors; BE, bronchial epithelium; CE, cranial epithelium; CNC, cranial neural crest; CNCP, cranial neural crest progenitors; (P), proliferative cells.

FIG. 68—Profiling spontaneously differentiated neurons from iNPs by single-cell RNA sequencing and target genes of candidate TFs by ChIP-seq. (A-E) UMAP reclustering of 4,162 neurons from clusters CN 1-3 of FIG. 66A. (A-D) Marker genes for general regions of the central nervous systems (A), newborn cortical excitatory neurons (B), neuronal subtypes (C), and cortical projection neurons (D) are shown. Colors indicate gene expression. (E) Neurons spontaneously differentiated from each candidate TF are highlighted. Colors indicate biological replicates, S1 and S2. (F) Top 3 de novo or known motifs identified using HOMER motif analysis. The names of the TFs with the closest matching motifs, indicating potential cofactors of candidate TFs, and the associated P-values of enrichment are listed. (G) Heatmap showing percentage of NP-specific TFs or genes that had candidate TF ChIP peaks within 10 kb of the annotated transcriptional start site (TSS). (H-I) Overlap of NP-specific genes that had candidate TF ChIP peaks within 10 kb of the TSS and were differentially expressed (t-test q-value<0.05 with FDR correction) upon candidate TF overexpression. Genes that were shared between candidate TFs are shown in (H), with blue regions indicating overlap, and genes unique to each candidate TF are shown in (I).

FIG. 69—Combining RFX4 with dual SMAD inhibition produces homogenous iNPs. (A) Schematic for different media conditions (M1-M8) tested. SMAD inhibitors dorsomorphin (DM) and SB-431542 (SB) were added to the media at the indicated concentrations. mTeSR stem cell media was changed to different NP media (NP, EB, and DS; see Methods) over 7 days of differentiation. (B) Heatmaps showing expression of neuron marker genes TUJ1 and MAP2 relative to GAPDH control in cells from iNPs that have undergone spontaneous neurogenesis for 2 or 4 weeks. iNPs were differentiated for 5 or 7 days using each of the media conditions in (A) and seeded at low or high densities prior to spontaneous neurogenesis. Colors represent mean expression from n=4 biological replicates. (C) Same as (A), for additional media conditions tested. (D) Same as (B), for the media conditions shown in (C). (E-K) Profiling of iNPs derived using different iNP differentiation methods by scRNA-seq. RFX4-DS-iNPs were produced by combining RFX4 overexpression with dual SMAD inhibition, EB-iNPs were produced using the embryoid body protocol (8), and DS-iNPs were produced using the dual SMAD inhibition protocol (7). Data represents n=2 batch replicates per method with 15,211 RFX4-DS-iNPs, 11,148 EB-iNPs, and 16,421 DS-iNPs. (E) UMAP clustering of scRNA-seq data with colors indicating Louvain clusters. (F) Dot plot showing marker genes for each cluster. Circle size indicates percentage of cells expressing the gene in the given cluster and color indicates the mean expression value. (G-H) Box plots showing intra-(G) or inter-(H) replicate Euclidean distances between cells. Whiskers indicate the 5^{th} and 95^{th} percentiles. (I) Data as in (E), highlighting cells derived from each differentiation method. Colors indicate batch replicates, S1 and S2. (J) Heatmap showing the percentage of cells from each batch replicate that were grouped into each cluster. (K) Data as in (E), colored by marker gene expression. NP, neural progenitors; CN, CNS neurons; CNC, cranial neural crest.

FIG. 70—Characterization of iNPs produced by combining RFX4 with dual SMAD inhibition. ScRNA-seq profiling of 26,111 cells that have been spontaneously differentiated from iNPs. iNPs were produced by combining RFX4 overexpression with dual SMAD inhibition and spontaneously differentiated for 4 or 8 weeks. Data represents n=2 biological replicates per timepoint. (A-B) UMAP clustering of scRNA-seq data. (A) Colors indicate expression of marker genes for major cell types. (B) Cells from each time point are highlighted. Colors indicate biological replicates, S1 and S2. (C) Heatmap showing the percentage of cells from each biological replicate that were grouped into each cluster from FIG. 5D. (D-G) UMAP clustering of scRNA-seq data. Marker genes for general regions of the central nervous systems (D), radial glia subtypes (E), neuronal subtypes (F), and GABAergic interneuron subtypes (G) are shown. Colors indicate gene expression. CN, CNS neurons; RG, radial glia; MNG, meninges; (P), proliferative cells.

FIG. 71—Modeling neurodevelopmental disorders using RFX4-iNPs with DYRK1A perturbation. (A) Schematic of disease modeling by perturbing DYRK1A expression. Human induced pluripotent stem cells (iPSCs) are transduced with Cas9 and sgRNAs or ORF to knockout or overexpress DYRK1A, respectively. RFX4 is then transiently overexpressed for 1 week to differentiate iPSCs into iNPs, which then spontaneously differentiate for 8 weeks following withdrawal of dox and growth factors. Effects of DYRK1A perturbation were characterized using bulk RNA sequencing, EdU labeling, immunostaining, or electrophysiology. dox, doxycycline; EGF, epidermal growth factor; FGF, fetal growth factor. (B) Percent indels in RFX4-iNPs transduced with DYRK1A KO sgRNAs. n=3. (C) DYRK1A expression measured using qPCR probes targeting the endogenous sequence or the codon-optimized ORF sequence. n=4. (D-E) Western blot of DYRK1A at 7 days after transduction with Cas9 and DYRK1A KO sgRNAs (D) or DYRK1A ORF (E). (F-H) Volcano plots showing the number of genes that were significantly differentially expressed (t-test q-value<0.05 with FDR correction) and had an absolute log 2 fold change relative to control that was greater than 1 for DYRK1A KO sgRNA 1 (F), KO sgRNA 2 (G), and ORF (H) conditions. For a full list of genes, see Table 17. The KO sgRNAs 1 and 2 conditions were compared to both NT sgRNAs. The ORF condition was compared to GFP control. (I) Venn diagram summarizing the significantly differentially expressed genes in (F-H). (J) Heatmap of genes that were significantly differentially expressed (t-test q-value<0.5 with FDR correction) depending on the dosage of DYRK1A. Genes are annotated with broad categories of gene function relevant to neural development. n=3. (K) Representative images of MAP2 staining during spontaneous differentiation for NT sg1 and DYRK1A KO sg2. Scale bar, 100 μm. Values represent mean±SEM. sg, single guide RNA; KO, knockout; NT, nontargeting. *P<0.05; ND, not detected.

FIG. 72—Characterization of DYRK1A perturbations in RFX4-iNP differentiated neurons by electrophysiology. (A) Representative electrophysiology traces for neurons with or without evoked action potentials (AP) and spontaneous excitatory postsynaptic currents (EPSCs). (B) Proportion of neurons with or without AP and EPSCs for different DYRK1A perturbations from n=31-45 neurons. (C-D) Intrinsic membrane (C) and action potential (D) properties measured using electrophysiology for different DYRK1A perturbations from n=12-36 neurons with evoked action potentials. Values represent mean±SEM. *P<0.05.

FIG. 73—Joint profiling of chromatin accessibility and gene expression on a subset of TF ORFs. (A) Violin plots showing distribution of UMIs and genes per cell for scRNA-seq from the joint profiling dataset. (B) Violin plots showing distribution of UMIs and fraction of reads in the top 500,000 peaks per cell for scATAC-seq from the joint profiling dataset. (C) Representative fragment histogram for scATAC-seq data using the first two megabases of chromosome 1. (D) Transcriptional start site (TSS) enrichment score for scATAC-seq data. (E) RNA (left) and ATAC (right) UMAP of 69,085 cells overexpressing 198 TF isoforms. Colors indicate clusters identified by the small local moving (SLM) algorithm. (F) Distribution of cells from day 4 or day 7 of TF overexpression in each of the clusters from FIG. 5A. Clusters with >30% cells from either time point are indicated with asterisks. (G) Weighted nearest neighbor (WNN) UMAP of joint profiling data from FIG. 46A, colored by diffusion pseudotime. (H) Violin plots comparing diffusion pseudotimes of each time point. (I) Heat map showing significance of the top nominated regulators for each cluster. Top regulators were nominated by evaluating motif enrichment in ATAC peaks with significant peak-gene associations in each cluster. TFs that were identified as top ORFs and regulators are labeled in blue.

FIG. 74—Combinatorial TF screening identifies TF combinations with similar expression profiles. (A) UMAP of scRNA-seq profiles from hESCs overexpressing 57 combinations of 10 TF ORFs for 7 days. Colors indicate Louvain clusters. (B) Heat map showing percentage of cells with the indicated TF combination for each cluster. Percentages are determined by normalizing to the total number of cells with the TF ORF in the combinatorial dataset. (C) Heat map showing pairwise Pearson correlation between mean expression profiles of each TF combination. TF combinations are ordered by hierarchical clustering.

FIG. 75—Fitting expression profiles of TF combinations with linear regression. (A-C) Heat maps showing the coefficient weights (A-B) and score (C) for linear regression. Single TF expression profiles were fitted to model each measured double TF profile by performing linear regression with an interaction term on the mean expression profiles. (D) Annotated relationships for each TF combination based on the fitted linear regression coefficients. (E) Heat maps showing average expression profile of double TFs with those of respective single TFs for example combinations with annotated relationships.

FIG. 76—Predicting TF combinations using the TF Atlas. (A-F) Percent accuracy for different approaches to predict TFs for double (A-C) or triple (D-F) TF combinations. Single TF expression profiles from the TF Atlas were averaged or fitted with linear regression models against measured double or triple TF expression profiles. TF combinations were ranked by the fit to the measured combinatorial TF profile. The top combinations were evaluated for accuracy. For comparison to the single TF profiles from the combinatorial TF screen dataset, prediction accuracy for the 10 corresponding TFs from the TF Atlas are shown (A,D). To reduce the number of possible combinations, TFs were grouped into 30 (B,E) or 51 (C,F) clusters based on expression profile similarity. (G-L) Prediction results for triple TF profiles. Known combinations (G) or predicted combinations for hepatoblasts (H), bronchiolar and alveolar epithelial cells (I), metanephric cells (J), vascular endothelial cells (K), and trophoblast giant cells (L) are shown. To expand the number of known combinations, parts of known combinations with more than 3 TFs were included for ENS neurons and cardiomyocytes. TF combinations were ranked by the gene signature scores for each respective cell type. As gene signature scores were discrete, the percentile ranks were reported as ranges. For predicted combinations, TFs that are part of known combinations, developmentally critical, or specifically expressed in the target cell types are indicated in blue.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “multiplicity of infection” (MOI) as used herein refers to the ratio of agents (e.g. vector, transcription factors) introduced to target cells (e.g. stem cell, radial glia). In certain embodiments, MOI can refer to viral vectors used to introduce an agent.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The ability to engineer cell types of interest has advanced basic research and has therapeutic potential, but is currently limited to a small number of cell types. Transcription factors (TFs) regulate gene programs, thereby controlling diverse cellular processes and cell states. Although overexpression of transcription factors (TFs) has been shown to efficiently convert one cell type to another, the process of discovering the right TFs is time-intensive and low-throughput.

The ability to engineer any cell type of interest has the potential to advance our understanding of biological processes and capability to treat disease¹⁻⁵. Despite this, currently only a few cell types can be generated efficiently and consistently^{1,2,4,5}. Overexpression of transcription factors (TFs) can be used to engineer cell fates, and TFs have been shown to rapidly and efficiently generate many different cell types, including neurons and skeletal muscle cells⁶⁻¹². For example, overexpressing either NEUROD1 or NEUROG2 can efficiently and rapidly differentiate hESCs into cortical neurons (Zhang Y, et al., Rapid single-step induction of functional neurons from human pluripotent stem cells. Neuron. 2013; 78(5):785-98). As TFs use endogenous regulatory pathways to drive differentiation, mimicking natural development, this approach may produce higher fidelity models while illuminating aspects of cellular development. Although overexpression of transcription factors (TFs) has been shown to efficiently convert one cell type to another, the process of discovering TFs that can direct differentiation into a desired cell type (cellular engineering) is time-intensive and low-throughput, limiting the number of transformative TFs that have been identified. Typically, candidate TFs are overexpressed individually or in specific combinations. Cells produced from independent perturbations are evaluated for similarity with the target cell type using discrete assays. This costly and time-consuming process has restricted the TFs tested per cell type to those predicted from prior studies (5-25 TFs on average), thus limiting the number of novel TFs that have been identified for cellular engineering.

To achieve a comprehensive understanding of TFs and their respective programs, Applicants developed a platform for high-throughput, systematic TF ORF overexpression that leverages barcodes for pooled screening. Applicants created a library of all annotated human TF splice isoforms (1,836 genes encoding 3,548 isoforms) and applied it to build a TF Atlas charting expression profiles in human embryonic stem cells (hESCs) overexpressing each TF. The comprehensive TF Atlas allowed systematic investigation and generalized observations, showing that 27% of TF genes could function as “master regulators” that induce differentiation when overexpressed in hESCs. Applicants mapped TF-induced expression profiles to reference cell types and validated candidate TFs for generation of diverse cell types, spanning all three germ layers and trophoblasts. Further targeted screens with a subset of the library allowed Applicants to create a tailored cellular disease model and integrate mRNA expression and chromatin accessibility data to identify downstream regulators. Finally, Applicants predicted the effects of TF combinations, demonstrated the validity of the predictions in a combinatorial TF overexpression dataset, and showed how to predict combinations of TFs that could produce target profiles of reference cell types, reducing the combinatorial search space for experiments. The TF atlas provides a comprehensive overview of gene regulatory networks and a roadmap for further understanding developmental trajectories and guiding cellular engineering efforts.

Applicants also provide different selection methods to enrich for expression of different numbers of marker genes that define the target cell type (reporter assay, Flow-FISH, and scRNA-seq).

Applicants applied the library to differentiation of human embryonic stem cells (hESCs) into neural progenitors (NPs). Applicants identified four TFs (RFX4, NFIB, PAX6, and ASCL1) that produced induced NPs (iNPs) that spontaneously differentiate into an array of central nervous system (CNS) cell types. RFX4-iNPs gave rise to the highest proportion of CNS cell types and, when combined with dual SMAD inhibition, produced iNPs at >98% purity that differentiated into predominantly GABAergic neurons, opening up new avenues for studying this cell type.

In an exemplary case, 90 TF isoforms specifically expressed in a selected target cell type (neural progenitors) were selected using available expression data (Camp et al., 2015; Johnson et al., 2015; Llorens-Bobadilla et al., 2015; Pollen et al., 2015; Shin et al., 2015; Thomsen et al., 2016; Wu et al., 2010; Zhang et al., 2016) for screening neural progenitors (NPs). Applicants chose to differentiate hESCs into induced NPs (iNPs) because NPs are born early during development, and therefore overexpression of a single TF may be sufficient to differentiate NPs from hESCs, though none have been identified. In addition, current methods for producing NPs, embryoid body formation¹³ or dual SMAD inhibition¹⁴, are either low-throughput or produce variable differentiation results depending on the cell line¹⁵, respectively. Through pooled screening of 90 TF isoforms, Applicants found four novel TFs (RFX4, NFIB, PAX6, and ASCL1), each of which can produce functional iNPs within 1 week. The iNPs resemble the morphology, transcriptome signature, and functional capabilities of human fetal NPs. Applicants then applied the iNPs to model neurodevelopmental disorders. These results collectively demonstrate the feasibility of using pooled TF screening to produce a diverse array of cell types that could be tailored for specific applications.

Notably, although RFX4 has not been extensively studied in neural development, RFX4-derived iNPs spontaneously differentiated into the highest proportion of cell types in the central nervous system (CNS), highlighting the importance of performing unbiased TF screens. Applicants demonstrated that RFX4-derived iNPs can be used to model neurodevelopmental disorders. Applicants also identified transcription factors capable of differentiating stem cells into cardiomyocytes. The TF screening platform provides a generalizable approach for cellular programming that could expand our ability to generate desired cell types and elucidate the complex TF regulatory networks that govern cell type specification.

Embodiments disclosed herein provide for a screening platform and methods of screening for transcription factors (TFs) that drive differentiation of stem cells into target cell types. The stem cells may be induced pluripotent stem cells (also known as iPS cells or iPSCs). The iPSCs may be patient derived.

Embodiments disclosed herein also provide for a screening platform and methods of screening for transcription factors that drive transdifferentiation of cells into target cell types. In certain embodiments, transcription factors that differentiate stem cells into a target cell (e.g., progenitor cell) can be used to transdifferentiate cells of a different lineage to target cells. In certain embodiments, TFs that are expressed in progenitor cells can be used to transdifferentiate cells of one lineage into a target cell of a different lineage.

Embodiments disclosed herein also provide also provide for high throughput screening methods for identifying transcription factors that enhance or suppress tumor growth. In certain embodiments, a barcoded transcription factor library is introduced to a cancer cell line. After growing the cancer cell line (e.g., 2 weeks) the barcodes are sequenced and enriched and depleted barcodes are identified as compared to the barcodes present in the initial library. Enriched barcodes may indicate transcription factors that enhance tumor growth and depleted barcodes may indicate transcription factors that suppress tumor growth.

In certain embodiments, the screening platform is a high-throughput multiplex screening platform.

Embodiments disclosed herein also provide for methods of using transcription factors to drive differentiation of stem cells (e.g., iPSCs or hESCs) into target cell types (e.g., neural cell types, cardiomyocytes), providing a road map for the development of an array of in vitro human models (e.g., brain) that can be tailored for specific applications. Embodiments disclosed herein also provide for in vitro models of in vivo cell types for use in modelling development and disease. In certain embodiments, target cell types can be transferred to a subject in need thereof to regenerate a diseased or damaged tissue.

Embodiments disclosed herein also provide differentiating or transdifferentiating cells into target cells in vivo by targeted modulation of transcription factors or downstream targets. In certain embodiments, the targeted modulation of transcription factors can be used to regenerate, replenish or replace damaged or diseased cells in a subject in need thereof (e.g., heart cells, pancreatic β cells, eye cells, nervous system cells).

Embodiments disclosed herein also provide for modulating transcription factors that enhance tumor growth or that suppress tumor growth. In certain embodiments, transcription factors are modulated in a treatment regimen in a subject suffering from cancer. In certain embodiments, the treatment is targeted to tumors or sites of tumors.

Many methods of modulating transcription factors may be used. In certain embodiments, the activity of transcription factors can be enhanced (e.g., by modulation of TF phosphorylation sites). In certain embodiments, TFs are overexpressed. In certain embodiments, agents capable of enhancing expression or activity of transcription factors are used. In certain embodiments, agents capable of reducing expression or activity of transcription factors are used.

Applicants provide further examples of the screening methods to identify transcription factors required for differentiation of hESCs into radial glia, neural progenitors in the developing central nervous system that are capable of differentiating into neurons, astrocytes, and oligodendrocytes. Applicants further identify TFs required for differentiation of hESCs into cardiomyocytes. The present invention also advantageously provides for high-throughput methods of screening.

Applicants identified TFs that can differentiate hESCs into radial glia. Additionally, these candidate TFs can advantageously be applied to a high-throughput screening platform for identifying TFs that direct differentiation into specific cell types of interest (e.g., interneurons, pyramidal neurons, and oligodendrocytes). The screen can advantageously be used to identify TFs that differentiate radial glia into astrocytes. The screening platform can advance understanding of gene regulation in neural development and provide robust, scalable cellular models for studying the brain.

Finally, the methods of differentiation using the identified transcription factors can advantageously produce homogenous populations of target cells (e.g., neural progenitor cell populations).

### Screening Platforms

In certain embodiments, the present invention provides a screening platform for systematically identifying transcription factors (TFs) that drive differentiation of cells (e.g., pluripotent, stem cells, progenitor cells) into target cell types (e.g., neural cells, muscle cells, endocrine cells). In certain embodiments, the screening platform comprises pluripotent cells that are differentiated into target cells by overexpressing a plurality of transcription factors in the pluripotent cells. Over expression of transcription factors may be performed according to any method known in the art (e.g., introducing a vector encoding the transcription factor, introducing an agent capable of inducing expression of the endogenous gene, as described further herein). The screening platforms can provide a framework for the development of an array of in vitro human models that can be tailored for specific applications described herein. Further, the screening platform can be used to generate a transcription factor atlas, such that differential gene expression in cells differentiated using each individual transcription factor is identified. Thus, the atlas can be used to group TFs based on gene expression and to identify TFs for each target cell type. The gene expression profile generated by overexpressing single TFs in the TF Atlas can be used to predict expression profiles produced by overexpressing TF combinations (discussed further herein).

In certain embodiments, transcription factors may be selected for screening based on expression of the transcription factors in the target cell types or in progenitor cells for the target cell types. Non-limiting examples of transcription factors may be found in Tables 1, 3, 4 and 5. Cell type specific transcription factors are known in the art. Additionally, expression of transcription factors in a target cell type can be determined experimentally (e.g., by RNA sequencing).

An exemplary screening platform comprises one or more populations of pluripotent cells, a means to over express one or more transcription factors in the one or more populations of cells, and a means to identify target cells after differentiation of the cells. Each population of pluripotent cells may express a different transcription factor.

### Pooled Screening Platforms

In certain embodiments, TFs are screened for differentiation of stem cells into a target cell in a pooled screen, such that a library of transcription factors are introduced to a single population of stem cells and transcription factors able to differentiate the stem cells are identified. In certain embodiments, transcription factors are introduced such that each cell receives no more than one transcription factor or are introduced such that single cells receive one or more transcription factors (e.g., 2, 3, 4, 5 transcription factors). In certain embodiments, the pooled screening platform can be used to identify combinations of transcription factors required for differentiation into a target cell type.

An exemplary pooled screening platform comprises a single population of pluripotent cells, a means to over express one or more transcription factors in one or more cells in the population of cells, and a high throughput means to identify target cells (e.g., microscopy, FACS, Flow-FISH, single cell RNA-seq, or reporter gene) and the over expressed transcription factor introduced to generate the target cells (e.g., barcode). Each pluripotent cell in the pool may express a different transcription factor or combination of transcription factors.

In certain embodiments, barcodes are used to identify the transcription factor or modulating agent for the transcription factor introduced to a cell or population of cells. In certain embodiments, stem cells differentiated into target cells are enriched (e.g., sorted) and the barcodes identified in the enriched cells indicate the transcription factors introduced. Thus, transcription factors may be identified by determining the enrichment of barcodes in cells differentiated into target cells compared to barcodes in the starting library.

Nucleic acid barcode or barcode refer to a short sequence of nucleotides (for example, DNA or RNA) that is used as an identifier for an associated molecule, such as a target molecule and/or target nucleic acid (e.g., transcription factor). A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides and can be in single- or double-stranded form. In certain embodiments, the barcode is configured for amplification and subsequent sequencing. In certain embodiments, the barcode is expressed as a transcript (e.g., poly A tailed transcript) that can be identified using a method of RNA sequencing as described further herein. In certain embodiments barcoding uses an error correcting scheme (T. K. Moon, Error Correction Coding: Mathematical Methods and Algorithms (Wiley, New York, ed. 1, 2005)).

### Pluripotent Cells

### Stem Cells

Pluripotent cells may include any mammalian stem cell. As used herein, the term “stem cell” refers to a multipotent cell having the capacity to self-renew and to differentiate into multiple cell lineages. Mammalian stem cells may include, but are not limited to, embryonic stem cells of various types, such as murine embryonic stem cells, e.g., as described by Evans & Kaufman 1981 (Nature 292: 154-6) and Martin 1981 (PNAS 78: 7634-8); rat pluripotent stem cells, e.g., as described by lannaccone et al. 1994 (Dev Biol 163: 288-292); hamster embryonic stem cells, e.g., as described by Doetschman et al. 1988 (Dev Biol 127: 224-227); rabbit embryonic stem cells, e.g., as described by Graves et al. 1993 (Mol Reprod Dev 36: 424-433); porcine pluripotent stem cells, e.g., as described by Notarianni et al. 1991 (J Reprod Fertil Suppl 43: 255-60) and Wheeler 1994 (Reprod Fertil Dev 6: 563-8); sheep embryonic stem cells, e.g., as described by Notarianni et al. 1991 (supra); bovine embryonic stem cells, e.g., as described by Roach et al. 2006 (Methods Enzymol 418: 21-37); human embryonic stem (hES) cells, e.g., as described by Thomson et al. 1998 (Science 282: 1 145-1 147); human embryonic germ (hEG) cells, e.g., as described by Shamblott et al. 1998 (PNAS 95: 13726); embryonic stem cells from other primates such as Rhesus stem cells, e.g., as described by Thomson et al. 1995 (PNAS 92:7844-7848) or marmoset stem cells, e.g., as described by Thomson et al. 1996 (Biol Reprod 55: 254-259). In certain embodiments, the pluripotent cells may include, but are not limited to lymphoid stem cells, myeloid stem cells, neural stem cells, skeletal muscle satellite cells, epithelial stem cells, endodermal and neuroectodermal stem cells, germ cells, extraembryonic and embryonic stem cells, mesenchymal stem cells, intestinal stem cells, embryonic stem cells, and induced pluripotent stem cells (iPSCs).

As noted, prototype “human ES cells” are described by Thomson et al. 1998 (supra) and in U.S. Pat. No. 6,200,806. The scope of the term covers pluripotent stem cells that are derived from a human embryo at the blastocyst stage, or before substantial differentiation of the cells into the three germ layers. ES cells, in particular hES cells, are typically derived from the inner cell mass of blastocysts or from whole blastocysts. Derivation of hES cell lines from the morula stage has been documented and ES cells so obtained can also be used in the invention (Strelchenko et al. 2004. Reproductive BioMedicine Online 9: 623-629). As noted, prototype “human EG cells” are described by Shamblott et al. 1998 (supra). Such cells may be derived, e.g., from gonadal ridges and mesenteries containing primordial germ cells from fetuses. In humans, the fetuses may be typically 5-11 weeks post-fertilization.

In certain embodiments, mouse embryonic stem cells are used. In certain embodiments, mouse embryonic stem cells differentiated into a target cell may be transferred to a mouse to perform in vivo functional studies.

Human embryonic stem cells may include, but are not limited to the HUES66, HUES64, HUES3, HUES8, HUES53, HUES28, HUES49, HUES9, HUES48, HUES45, HUES1, HUES44, HUES6, H1, HUES62, HUES65, H7, HUES13, H9, and HUES63 cell lines. In certain embodiments, the stem cell is a human induced pluripotent stem cell (iPSC). In certain embodiments, the human iPSC is selected from the group consisting of 11a, PGP1, GM08330 (also known as GM8330-8), and Mito 210.

General techniques useful in the practice of this invention in cell culture and media uses are known in the art (e.g., Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991). The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO₂ and >95% humidity.

Methods related to culturing stem cells are also useful in the practice of this invention (see, e.g., “Teratocarcinomas and embryonic stem cells: A practical approach” (E. J. Robertson, ed., IRL Press Ltd. 1987); “Guide to Techniques in Mouse Development” (P. M. Wasserman et al. eds., Academic Press 1993); “Embryonic Stem Cells: Methods and Protocols” (Kursad Turksen, ed., Humana Press, Totowa N.J., 2001); “Embryonic Stem Cell Differentiation in vitro” (M. V. Wiles, Meth. Enzymol. 225: 900, 1993); “Properties and uses of Embryonic Stem Cells: Prospects for Application to Human Biology and Gene Therapy” (P. D. Rathjen et al., al., 1993). Differentiation of stem cells is reviewed, e.g., in Robertson. 1997. Meth Cell Biol 75: 173; Roach and McNeish. 2002. Methods Mol Biol 185: 1-16; and Pedersen. 1998. Reprod Fertil Dev 10: 31). For further elaboration of general techniques useful in the practice of this invention, the practitioner can refer to standard textbooks and reviews in cell biology, tissue culture, and embryology (see, e.g., Culture of Human Stem Cells (R. Ian Freshney, Glyn N. Stacey, Jonathan M. Auerbach—2007); Protocols for Neural Cell Culture (Laurie C. Doering—2009); Neural Stem Cell Assays (Navjot Kaur, Mohan C. Vemuri—2015); Working with Stem Cells (Henning Ulrich, Priscilla Davidson Negraes—2016); and Biomaterials as Stem Cell Niche (Krishnendu Roy—2010)). In certain embodiments, stem cells are spontaneously differentiated or directed to differentiate (see, e.g., Amit and Itskovitz-Eldor, Derivation and spontaneous differentiation of human embryonic stem cells, J Anat. 2002 March; 200(3): 225-232). For further methods of cell culture solutions and systems, see International Patent Publication No. WO 2014/159356A1.

### Induced Pluripotent Cells

In certain embodiments, iPSCs or iPSC cell lines are used to identify transcription factors for differentiation of target cells. iPSCs advantageously can be used to generate patient specific models and cell types. iPSCs are a type of pluripotent stem cell that can be generated directly from adult cells. Further, because embryonic stem cells can only be derived from embryos, it has so far not been feasible to create patient-matched embryonic stem cell lines.

Various strategies can be used to induce pluripotency, or increase potency, in cells (Takahashi, K., and Yamanaka, S., Cell 126, 663-676 (2006); Takahashi et al., Cell 131, 861-872 (2007); Yu et al., Science 318, 1917-1920 (2007); Zhou et al., Cell Stem Cell 4, 381-384 (2009); Kim et al., Cell Stem Cell 4, 472-476 (2009); Yamanaka et al., 2009; Saha, K., Jaenisch, R., Cell Stem Cell 5, 584-595 (2009)), and improve the efficiency of reprogramming (Shi et al., Cell Stem Cell 2, 525 20 528 (2008a); Shi et al., Cell Stem Cell 3, 568-574 (2008b); Huangfu et al., Nat Biotechnol 26, 795-797 (2008a); Huangfu et al., Nat Biotechnol 26, 1269-1275 (2008b); Silva et al., Plos Bio 6, e253. doi: 10.1371/journal. pbio. 0060253 (2008); Lyssiotis et al., PNAS 106, 8912-8917 (2009); Ichida et al., Cell Stem Cell 5, 491-503 (2009); Maherali, N., Hochedlinger, K., Curr Biol 19, 1718-1723 (2009b); Esteban et 25 al., Cell Stem Cell 6, 71-79 (2010); and Feng et al., Cell Stem Cell 4, 301-3 12 (2009)).

Generally, techniques for reprogramming involve modulation of specific cellular pathways, either directly or indirectly, using polynucleotide-, polypeptide and/or small molecule-based approaches (see, e.g., International Patent Publication No. WO 2012/087965A2). The developmental potency of a cell may be increased, for example, by contacting a cell with one or more pluripotency factors. “Contacting”, as used herein, can involve culturing cells in the presence of a pluripotency factor (such as, for example, small molecules, proteins, peptides, etc.) or introducing pluripotency factors into the cell. Pluripotency factors can be introduced into cells by culturing the cells in the presence of the factor, including transcription factors such as proteins, under conditions that allow for introduction of the transcription factor into the cell. See, e.g., Zhou H et al., Cell Stem Cell. 2009 May 8; 4(5):381-4; International Patent Publication No. WO 2009/117439. Introduction into the cell may be facilitated, for example, using transient methods, e.g., protein transduction, microinjection, non-integrating gene delivery, mRNA transduction, etc., or any other suitable technique. In some embodiments, the transcription factors are introduced into the cells by expression from a recombinant vector that has been introduced into the cell, or by incubating the cells in the presence of exogenous transcription factor polypeptides such that the polypeptides enter the cell. In particular embodiments, the pluripotency factor is a transcription factor. Exemplary transcription factors that are associated with increasing, establishing, or maintaining the potency of a cell include, but are not limited to Oct-3/4, Cdx-2, 15 Gbx2, Gsh1, HesX1, HoxA10, HoxA 11, HoxB1, Irx2, Isl1, Meis1, Meox2, Nanog, Nkx2.2, Onecut, Otx1, Oxt2, Pax5, Pax6, Pdx1, Tcf1, Tcf2, Zfhxlb, Klf-4, Atbf1, Esrb, Genf, Jarid2, Jmjd1a, Jmjd2c, Klf-3, Klf-5, Mel-18, Myst3, Nac1, REST, Rex-i, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Lef1, Sox15, Sox6, Tcf-7, Tcf7ll, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPP, mafa, Eomes, Tbx-3; Rfx4, Stat3, Stella, and UTF-1. Exemplary transcription factors include Oct4, Sox2, Klf4, c-Myc, and Nanog.

Small molecule reprogramming agents are also pluripotency factors and may also be employed in the methods of the invention for inducing reprogramming and maintaining or increasing cell potency. In some embodiments of the invention, one or more small molecule reprogramming agents are used to induce pluripotency of a somatic cell, increase or maintain the potency of a cell, or improve the efficiency of reprogramming. In some embodiments, small molecule reprogramming agents are employed in the methods of the invention to improve the efficiency of reprogramming. Improvements in efficiency of reprogramming can be measured by (1) a decrease in the time required for reprogramming and generation of pluripotent cells (e.g., by shortening the time to generate pluripotent cells by at least a day compared to a similar or same process without the small molecule), or alternatively, or in combination, (2) an increase in the number of pluripotent cells generated by a particular process (e.g., increasing the number of cells reprogrammed in a given time period by at least 10%, 30%, 50%, 100%, 200%, 500%, etc. compared to a similar or same process without the small molecule). In some embodiments, a 2-fold to 20-fold improvement in reprogramming efficiency is observed. In some embodiments, reprogramming efficiency is improved by more than 20 fold. In some embodiments, a more than 100 fold improvement in efficiency is observed over the method without the small molecule reprogramming agent (e.g., a more than 100 fold increase in the number of pluripotent cells generated). Several classes of small molecule reprogramming agents may be important to increasing, establishing, and/or maintaining the potency of a cell. Exemplary small molecule reprogramming agents include, but are not limited to: agents that inhibit H3K9 methylation or promote H3K9 demethylation; agents that inhibit H3K4 demethylation or promotes H3K4 methylation; agents that inhibit histone deacetylation or promote histone acetylation; L-type Ca channel agonists; activators of the cAMP pathway; DNA methyltransferase (DNMT) inhibitors; nuclear receptor ligands; GSK3 inhibitors; MEK inhibitors; TGFP receptor/ALK5 inhibitors; HDAC inhibitors; Erk inhibitors; ROCK inhibitors; FGFR inhibitors; and PARP inhibitors. Exemplary small molecule reprogramming agents include GSK3 inhibitors; MEK inhibitors; TGFP receptor/ALK5 inhibitors; HDAC inhibitors; Erk inhibitors; and ROCK inhibitors.

In some embodiments of the invention, small molecule reprogramming agents are used to replace one or more transcription factors in the methods of the invention to induce pluripotency, improve the efficiency of reprogramming, and/or increase or maintain the potency of a cell. For example, in some embodiments, a cell is contacted with one or more small molecule reprogramming agents, wherein the agents are included in an amount sufficient to improve the efficiency of reprogramming. In other embodiments, one or more small molecule reprogramming agents are used in addition to transcription factors in the methods of the invention. In one embodiment, a cell is contacted with at least one pluripotency transcription factor and at least one small molecule reprogramming agent under conditions to increase, establish, and/or maintain the potency of the cell or improve the efficiency of the reprogramming process. In another embodiment, a cell is contacted with at least one pluripotency transcription factor and at least two, at least three, at least four, at least five, at least six, at least seven, at least eight, at least nine, or at least ten small molecule reprogramming agents under conditions and for a time sufficient to increase, establish, and/or maintain the potency of the cell or improve the efficiency of reprogramming. The state of potency or differentiation of cells can be assessed by monitoring the pluripotency characteristics (e.g., expression of markers including, but not limited to SSEA-3, SSEA-4, TRA-1-60, TRA-1-81, TRA-2-49/6E, Oct-3/4, Sox2, Nanog, GDF3, REX1, FGF4, ESG1, DPPA2, DPPA4, and hTERT).

### Introducing Transcription Factors

In certain embodiments, the screening platform may comprise an open reading frame (ORF) or cDNA encoding each transcription factor used in the screen (as used herein cDNA or ORF may be used interchangeably). A cDNA may be synthesized and cloned into a vector. A plurality of cDNAs may be cloned into a library of vectors, such that each transcription factor is represented in the library. Representative transcription factor libraries are known in the art (see, e.g., Yang et al., 2011, A public genome-scale lentiviral expression library of human ORFs Nature Methods 8, 659-66; and portals.broadinstitute.org/gpp/public/).

In certain embodiments, the screening platform may comprise an agent capable of overexpressing or modulating activity of endogenous transcription factors. In certain embodiments, the agent may be a CRISPR system. In certain embodiments, pluripotent cells are differentiated into target cells by introducing a CRISPR system targeting the endogenous loci encoding the transcription factors. In certain embodiments, the CRISPR system comprises a functional domain that is targeted to the endogenous loci encoding the transcription factors. The functional domain may be a transcriptional activator or repressor (see, e.g., Konermann et al. “Genome-scale transcriptional activation by an engineered CRISPR-Cas9 complex” Nature. 2014 Dec. 10. doi: 10.1038/nature14136; Qi, L. S., et al. (2013). “Repurposing CRISPR as an RNA-guided platform for sequence-specific control of gene expression”. Cell. 152 (5): 1173-83; and Gilbert, L. A., et al., (2013). “CRISPR-mediated modular RNA-guided regulation of transcription in eukaryotes”. Cell. 154 (2): 442-51). In certain embodiments, a functional domain is targeted to a genomic locus encoding a transcription factor using a guide sequence that includes one or more aptamer sequences. In particular embodiments, this is ensured by the use of adaptor protein/aptamer combinations that exist within the diversity of bacteriophage coat proteins. Examples of such coat proteins include but are not limited to: MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. In particular embodiments, the aptamer is a minimal hairpin aptamer which selectively binds dimerized MS2 bacteriophage coat proteins in mammalian cells and is introduced into the guide molecule, such as in the stemloop and/or in a tetraloop. In these embodiments, the functional domain is fused to MS2 (see, e.g., Konermann et al., Nature 2015, 517(7536): 583-588).

In certain embodiments, the arrayed screening platform can utilize multiwell plates to introduce individual transcription factors or an agent capable of modulating said transcription factors to populations of pluripotent cells. As used throughout the specification, reference to introducing transcription factors can refer to overexpressing the transcription factor from a vector or introducing an agent capable of modulating said transcription factor (e.g., CRISPR system targeting the transcription factor). Thus, each well of the multiwell plate may be configured for overexpression of a single transcription factor or combination of multiple transcription factors.

In certain embodiments, transcription factors may be introduced to individual cells by nanowires (see e.g., Shalek et al., Vertical silicon nanowires as a universal platform for delivering biomolecules into living cells, PNAS, Volume 107, Issue 1870 February, 2010). This modality enables one to assess the phenotypic consequences of introducing a broad range of biological effectors (DNAs, RNAs, peptides, proteins, and small molecules) into almost any cell type. In certain embodiments, the nanowires may be configured on a microarray format. In certain embodiments, the microarray may be configured for overexpressing transcription factors in a site-specific fashion. In certain embodiments, the array may be coupled with live-cell imaging.

### Vectors

In certain embodiments, vectors are used to overexpress or modulate expression of transcription factors. Vectors for introducing CRISPR systems are described further herein.

The term “vector” generally denotes a tool that allows or facilitates the transfer of an entity from one environment to another. More particularly, the term “vector” as used throughout this specification refers to nucleic acid molecules to which nucleic acid fragments (cDNA) may be inserted and cloned, i.e., propagated. Hence, a vector is typically a replicon, into which another nucleic acid segment may be inserted, such as to bring about the replication of the inserted segment in a defined host cell or vehicle organism.

A vector thus typically contains an origin of replication and other entities necessary for replication and/or maintenance in a host cell. A vector may typically contain one or more unique restriction sites allowing for insertion of nucleic acid fragments. A vector may also preferably contain a selection marker, such as, e.g., an antibiotic resistance gene or auxotrophic gene (e.g., URA3, which encodes an enzyme necessary for uracil biosynthesis or TRP1, which encodes an enzyme required for tryptophan biosynthesis), to allow selection of recipient cells that contain the vector. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

Expression vectors are generally configured to allow for and/or effect the expression of nucleic acids (e.g., cDNA, CRISPR system) introduced thereto in a desired expression system, e.g., in vitro, in a host cell, host organ and/or host organism. For example, the vector can express nucleic acids functionally or operatively linked to regulatory element(s) and hence the regulatory element(s) drive expression. The promoter(s) can be constitutive promoter(s) and/or conditional promoter(s) and/or inducible promoter(s). In certain embodiments, the vectors comprise regulatory sequences for inducible expression of cDNAs encoding transcription factors. Thus, expression of the transcription factors in cells can induced at particular time points after introducing the vectors. Inducible expression systems are known in the art and may include, for example, Tet on/off systems (see, e.g., Gossen et al., Transcriptional activation by tetracyclines in mammalian cells. Science. 1995 Jun. 23; 268(5218):1766-9).

In certain example embodiments, the vectors disclosed herein may further encode an epitope tag in frame with the transcription factors for use in downstream assessment of protein expression and TF abundance in cell populations respectively. Epitope tags provide high sensitivity and specificity in detection by specific antigen binding molecules (e.g., antibodies, aptamers). Exemplary epitope tags include, but are not limited to, Flag, CBP, GST, HA, HBH, MBP, Myc, polyHis, S-tag, SUMO, TAP, TRX, or V5.

Vectors may include, without limitation, plasmids (which refer to circular double stranded DNA loops which, in their vector form are not bound to the chromosome), episomes, phagemids, bacteriophages, bacteriophage-derived vectors, bacterial artificial chromosomes (BAC), yeast artificial chromosomes (YAC), P1-derived artificial chromosomes (PAC), transposons, cosmids, linear nucleic acids, viral vectors, etc., as appropriate. A vector can be a DNA or RNA vector. A vector can be a self-replicating extrachromosomal vector or a vector which integrates into a host genome, hence, vectors can be autonomous or integrative.

The term “viral vectors” refers to the use as viruses, or virus-associated vectors as carriers of the nucleic acid construct into the cell. Constructs may be integrated and packaged into non-replicating, defective viral genomes like adenovirus, adeno-associated virus (AAV), or herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g., EPV and EBV vectors.

Methods for introducing nucleic acids, including vectors, expression cassettes and expression vectors, into cells (e.g., transfection, transduction or transformation) are known to the person skilled in the art, and may include calcium phosphate co-precipitation, electroporation, micro-injection, protoplast fusion, lipofection, exosome-mediated transfection, transfection employing polyamine transfection reagents, bombardment of cells by nucleic acid-coated tungsten micro projectiles, viral particle delivery, etc.

### Identification of Target Cells

In certain embodiments, differentiation of pluripotent cells is monitored. In certain embodiments, differentiation of pluripotent cells is monitored by microscopy. The screening method may further be combined with live cell imaging to monitor differentiation upon overexpression of transcription factors. The screening method may also be combined with FACS or ELISA assays to determine cells expressing markers specific for differentiated cell types. Additionally, methods of detecting target cell specific markers may include detecting reporter genes linked to marker genes, FISH, Flow-FISH, RNA sequencing, single cell RNA sequencing, quantitative RT-PCR, or western blot. In preferred embodiments, a pooled screen uses three different selection methods to enrich for cells that express one or more marker genes that define the target cell type; reporter assay, Flow-FISH, and scRNA-seq. In preferred embodiments, each transcription factor is associated with a unique barcode sequence that can be detected using sequencing.

### Reporter Genes

In certain embodiments, differentiated target cells can be identified and enriched from a pool of cells using a detectable marker (i.e., high throughput means to identify target cells). In certain embodiments, the pooled screening platform uses detectable markers associated with marker genes specific to target cells to identify transcription factors.

In certain embodiments, the detectable marker is integrated into a genomic locus in the pool of cells such that the detectable marker is under control of the regulatory sequences for a target cell specific marker gene. In other words, a polynucleotide sequence encoding a detectable marker is integrated into a genomic locus encoding a marker gene, such that the marker gene and detectable marker are under control of the regulatory sequences for the marker gene and upon activation of the marker gene the detectable marker is co-expressed. In certain embodiments, the marker gene and detectable marker are expressed as separate proteins to avoid the detectable marker from interfering with proper protein folding and function of the marker gene. Thus, the detectable marker can be used to monitor activation of the marker gene to indicate differentiation into a target cell type. Thus, the present invention also provides for a population of pluripotent cells comprising a detectable marker integrated into an endogenous marker gene specific for a target cell.

Integration of the detectable marker gene at a genomic locus can be performed using known methods in the art. In certain embodiments, a donor construct is used to integrate a polynucleotide sequence encoding the detectable marker. In certain embodiments, the donor construct may comprise a nucleotide sequence encoding: a detectable marker, and optionally, a resistance gene operably linked to a separate regulatory sequence. Cells having the donor construct integrated can be selected based on fluorescence of the detectable marker. Cells having the donor construct integrated can be selected based on selection of cells expressing the resistance gene. The cells can be further selected by determining the integration site of the donor construct.

Selectable markers are known in the art and enable screening for targeted integrations. Examples of selectable markers include, but are not limited to, antibiotic resistance genes, such as beta-lactamase, neo, FabI, URA3, cam, tet, blasticidin, hyg, puromycin and the like. A selectable marker useful in accordance with the invention may be any selectable marker appropriate for use in a eukaryotic cell, such as a mammalian cell, or more specifically a human cell. One of skill in the art will understand and be able to identify and use selectable markers in accordance with the invention.

In certain embodiments, the donor construct is a plasmid, vector, PCR product, or synthesized polynucleotide sequence. In certain embodiments, the donor construct is modified to increase stability or to increase efficiency of integration into a genomic locus. In certain embodiments, the donor construct is modified by a 5′ and/or 3′ phosphorylation modification. In certain embodiments, the donor construct is modified by one or more internal or terminal PTO modifications. Phosphorothioate (PTO) modifications are used to generate nuclease resistant oligonucleotides. In PTO oligonucleotides, a non-bridging oxygen is replaced by a sulfur atom. Therefore, PTOs are also known as “S-oligos”. Phosphorothioate can be introduced to an oligonucleotide at the 5′- or 3′-end to inhibits exonuclease degradation and internally to limit the attack by endonucleases. In certain embodiments, the donor construct is obtained using PCR amplification and the 5′ phosphorylation is introduced using 5′ phosphorylated primers.

In certain embodiments, a genetic modifying agent is used to target the donor construct sequence to the correct genomic location (e.g., CRISPR, TALEN, Zinc finger protein, meganuclease).

In certain embodiments, a method of tagging genes in cells uses a donor template having homology arms that can be integrated at a target locus in the genome of a cell using homology dependent based repair mechanisms. In certain embodiments, a method of tagging genes in cells uses a generic donor template that can be integrated at any target locus in the genome of a cell using homology independent based repair mechanisms. In certain embodiments, gene tagging uses a CRISPR system. In certain embodiments, gene tagging uses a system that alleviates the need for homology templates. Previous reports using zinc-finger nucleases, TALE effector nucleases or CRISPR-Cas9 technology have shown that plasmids containing an endonuclease cleavage site can be integrated in a homology-independent manner and any of these methods may be used for constructing the tagged pluripotent population of cells of the present invention (see, e.g., Lackner, D. H. et al. A generic strategy for CRISPR-Cas9-mediated gene tagging. Nat. Commun. 6:10237 doi: 10.1038/ncomms10237 (2015); Auer, et al., Highly efficient CRISPR/Cas9-mediated knock-in in zebrafish by homology-independent DNA repair. Genome Res. 24, 142-153 (2014); Maresca, et al., Obligate ligation-gated recombination (ObLiGaRe): custom-designed nuclease-mediated targeted integration through nonhomologous end joining. Genome Res. 23, 539-546 (2013); and Cristea, S. et al., In vivo cleavage of transgene donors promotes nuclease-mediated targeted integration. Biotechnol. Bioeng. 110, 871-880 (2013)).

In certain embodiments, cells are tagged by introducing a ribonucleoprotein complex (RNP) comprising a donor sequence, guide sequences targeting a genomic locus and a CRISPR system. Delivery of CRISPR RNP complexes is described further herein. For example, the RNP complexes may be delivered to a population of cells by transfection.

In certain embodiments, the detectable marker is integrated downstream of the marker gene. In certain embodiments, the detectable marker is integrated upstream of the marker gene.

In certain embodiments, the detectable marker is separated from the marker gene by a ribosomal skipping site. Ribosomal ‘skipping’ refers to generating more than one protein during translation where a specific sequence in the nascent peptide chain prevents the ribosome from creating the peptide bond with the next proline. Translation continues and gives rise to a second chain. This mechanism results in apparent co-translational cleavage of the polyprotein. This process is induced by a ‘2A-like’, or CHYSEL (cis-acting hydrolase element) sequence. In other words, a normal peptide bond is impaired at the site, resulting in two discontinuous protein fragments from one translation event.

In certain embodiments, the detectable marker is a fluorescent protein such as green fluorescent protein (GFP), enhanced green fluorescent protein (EGFP), red fluorescent protein (RFP), blue fluorescent protein (BFP), cyan fluorescent protein (CFP), yellow fluorescent protein (YFP), miRFP (e.g., miRFP670, see, Shcherbakova, et al., Nat Commun. 2016; 7: 12405), mCherry, tdTomato, DsRed-Monomer, DsRed-Express, DSRed-Express2, DsRed2, AsRed2, mStrawberry, mPlum, mRaspberry, HcRed1, E2-Crimson, mOrange, mOrange2, mBanana, ZsYellow1, TagBFP, mTagBFP2, Azurite, EBFP2, mKalamal, Sirius, Sapphire, T-Sapphire, ECFP, Cerulean, SCFP3A, mTurquoise, mTurquoise2, monomelic Midoriishi-Cyan, TagCFP, niTFP1, Emerald, Superfolder GFP, Monomeric Azami Green, TagGFP2, mUKG, mWasabi, Clover, mNeonGreen, Citrine, Venus, SYFP2, TagYFP, Monomeric Kusabira-Orange, mKOk, mK02, mTangerine, mApple, mRuby, mRuby2, HcRed-Tandem, mKate2, mNeptune, NiFP, mkeima Red, LSS-mKate1, LSS-mkate2, mBeRFP, PA-GFP, PAmCherry1, PATagRFP, TagRFP6457, IFP1.2, iRFP, Kaede (green), Kaede (red), KikGR1 (green), KikGR1 (red), PS-CFP2, mEos2 (green), mEos2 (red), mEos3.2 (green), mEos3.2 (red), PSmOrange, Dronpa, Dendra2, Timer, AmCyan1, or a combination thereof. In certain embodiments, the detectable marker is a cell surface marker. In other instances, the cell surface marker is a marker not normally expressed on the cells, such as a truncated nerve growth factor receptor (tNGFR), a truncated epidermal growth factor receptor (tEGFR), CD8, truncated CD8, CD19, truncated CD19, a variant thereof, a fragment thereof, a derivative thereof, or a combination thereof.

In certain embodiments, the signal of the detectable marker may be enhanced by using a fluorescently labeled antibody, antibody fragment, nanobody, or aptamer. The binding agent may be specific to the detectable marker.

### Flow-FISH

In certain embodiments, Flow FISH (fluorescent in-situ hybridization) is used to identify target cells in transcription factor screens. Flow FISH is a cytogenetic technique to quantify the copy number of RNA or specific repetitive elements in genomic DNA of whole cell populations via the combination of flow cytometry with cytogenetic fluorescent in situ hybridization staining protocols (see, e.g., C. P. Fulco et al., Activity-by-contact model of enhancer-promoter regulation from thousands of CRISPR perturbations. Nat Genet 51, 1664-1669 (2019); and Coillard A, Segura E. Visualization of RNA at the Single Cell Level by Fluorescent in situ Hybridization Coupled to Flow Cytometry. Bio Protoc. 2018; 8(12):e2892). The method provides for detecting marker genes for indicating differentiation of target cells using gene specific FISH probes and sorting the cells. In certain embodiments, multiple markers are used to increase specificity. Selecting for multiple reporter genes at the same time can narrow down target cell types because in certain embodiments one gene is not specific enough depending on the target cell type. Additionally, the assay is versatile in that reporter genes can be added or changed by applying different probes. Flow FISH combines FISH to fluorescently label mRNA of reporter genes and flow cytometry (see, e.g., Arrigucci et al., FISH-Flow, a protocol for the concurrent detection of mRNA and protein in single cells using fluorescence in situ hybridization and flow cytometry, Nat Protoc. 2017 June; 12(6):1245-1260. doi:10.1038/nprot.2017.039). In certain embodiments, the mRNA of reporter genes is fluorescently labeled; target cells are selected by flow cytometry; and TF barcodes are sequenced (e.g., amplified and then sequenced) to identify TFs enriched in the target cells. In certain embodiments, the marker genes are selected, such that they are specifically expressed only in the target cell. In this way, false positive selection or background is avoided. In certain embodiments, the assay is optimized to remove background fluorescence and to select for true positive cells.

### Single Cell RNA-seq

In certain embodiments, the invention provides for identifying transcription factors whose overexpression can differentiate stem cells or progenitor cells into target cells by using single cell sequencing methods. In certain embodiments, transcription factors are introduced to a population of cells and single cells are analyzed by single cell sequencing. The population of cells may be analyzed with or without an integrated detectable marker. The introduced transcription factors can be identified in cells having a gene signature or biological program of interest (e.g., signature characteristic of the target cell). As used herein a “signature” may encompass any gene or genes, protein or proteins, or epigenetic element(s) whose expression profile or whose occurrence is associated with a specific cell type, subtype, or cell state of a specific cell type or subtype within a population of cells. A gene signature as used herein, may thus refer to any set of up- and down-regulated genes that are representative of a cell type or subtype or cell state. In certain embodiments, transcription factors are introduced at a high MOI to identify combinations of transcription factors capable of inducing a signature or biological program characteristic of the target cell of interest.

The transcription factors introduced may be identified by a barcode associated with each transcription factor. The barcode may be expressed on a transcript capable of identification by RNA-seq (e.g., a poly-A tailed transcript including the barcode sequence). In certain embodiments, single cells can be analyzed for a target cell phenotype or target cell subtypes after introducing transcription factors identified by the screening methods described herein. Thus, single cell sequencing may be used for identification of transcription factors and for analysis of cells differentiated by overexpressing transcription factors.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International Patent Application No. PCT/US2015/049178, published as International Patent Publication No. WO 2016/040476 on Mar. 17, 2016; Klein et al., 2015, “Droplet Barcoding for Single-Cell Transcriptomics Applied to Embryonic Stem Cells” Cell 161, 1187-1201; International Patent Application No. PCT/US2016/027734, published as International Patent Publication No. WO 2016/168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International Patent Publication No. WO 2014 210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO 2017/164936 on Sep. 28, 2017; Patent Application No. PCT/US2018/060860, published as WO 2019/094984 on May 16, 2019; Patent Application No. PCT/US2019/055894, published as WO 2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

In certain embodiments, the invention involves single cell multimodal data. Multiomic review (see, e.g., Lee J, Hyeon D Y, Hwang D. Single-cell multiomics: technologies and data analysis methods. Exp Mol Med. 2020; 52(9):1428-1442. doi:10.1038/s12276-020-0420-2). In certain embodiments, SHARE-Seq (Ma, S. et al. Chromatin potential identified by shared single cell profiling of RNA and chromatin. bioRxiv 2020.06.17.156943 (2020) doi:10.1101/2020.06.17.156943) is used to generate single cell RNA-seq and chromatin accessibility data. In certain embodiments, CITE-seq (Stoeckius, M. et al. Simultaneous epitope and transcriptome measurement in single cells. Nat. Methods 14, 865-868 (2017)) (cellular proteins) is used to generate single cell RNA-seq and proteomics data. In certain embodiments, Patch-seq (Cadwell, C. R. et al. Electrophysiological, transcriptomic and morphologic profiling of single neurons using Patch-seq. Nat. Biotechnol. 34, 199-203 (2016)) is used to generate single cell RNA-seq and patch-clamping electrophysiological recording and morphological analysis of single neurons data (see, e.g., van den Hurk, et al., Patch-Seq Protocol to Analyze the Electrophysiology, Morphology and Transcriptome of Whole Single Neurons Derived From Human Pluripotent Stem Cells, Front Mol Neurosci. 2018; 11: 261).

### Transcription Factor Modules

In example embodiments, the invention provides for identifying transcription factors whose overexpression can differentiate stem cells or progenitor cells into target cells by using single cell sequencing methods. In example embodiments, selecting cells further comprises grouping one or more of the transcription factors into modules that alter expression of the same gene programs, such that transcription factors in the same modules are co-functional (i.e., function in similar pathways or have similar functions). As used herein the term “gene program” or “program” can be used interchangeably with “biological program”, “expression program”, “transcriptional program”, “expression profile”, or “expression program” and may refer to a set of genes that share a role in a biological function (e.g., an activation program, cell differentiation program, proliferation program). Biological programs can include a pattern of gene expression that result in a corresponding physiological event or phenotypic trait. Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Multiple biological programs may include the same gene, reflecting the gene's roles in different processes. Expression of a biological program may be regulated by a master switch, such as a transcription factor or chromatin modifier. As used herein, the term “topic” refers to a biological program. The biological program can be modeled as a distribution over expressed genes.

One method to identify cell programs is non-negative matrix factorization (NMF) (see, e.g., Lee D D and Seung H S, Learning the parts of objects by non-negative matrix factorization, Nature. 1999 Oct. 21; 401(6755):788-91). As an alternative, a generative model based on latent Dirichlet allocation (LDA) (Blei, D. M., Ng, A. Y., and Jordan, M. I. (2003). Latent Dirichlet allocation. J Mach Learn Res 3, 993-1022), or “topic modeling” may be created. Topic modeling is a statistical data mining approach for discovering the abstract topics that explain the words occurring in a collection of text documents. Originally developed to discover key semantic topics reflected by the words used in a corpus of documents (Dumais, S. T., Furnas, G. W., Landauer, T. K., and Harshman, R. (1990). Indexing by Latent Semantic Analysis. Journal of the American Society for Information Science 41, 391-407), topic modeling can be used to explore gene programs (“topics”) in each cell (“document”) based on the distribution of genes (“words”) expressed in the cell. A gene can belong to multiple programs, and its relative relevance in the topic is reflected by a weight. A cell is then represented as a weighted mixture of topics, where the weights reflect the importance of the corresponding gene program in the cell. Topic modeling using LDA has recently been applied to scRNA-seq data (see, e.g., Bielecki, Riesenfeld, Kowalczyk, et al., 2018 Skin inflammation driven by differentiation of quiescent tissue-resident ILCs into a spectrum of pathogenic effectors. bioRxiv 461228; and duVerle, D. A., Yotsukura, S., Nomura, S., Aburatani, H., and Tsuda, K. (2016). CellTree: an R/bioconductor package to infer the hierarchical structure of cell populations from single-cell RNA-seq data. BMC Bioinformatics 17, 363). Other approaches include word embeddings. Identifying cell programs can recover cell states and bridge differences between cells. Single cell types may span a range of continuous cell states (see, e.g., Shekhar et al., Comprehensive Classification of Retinal Bipolar Neurons by Single-Cell Transcriptomics Cell. 2016 Aug. 25; 166(5):1308-1323.e30; and Bielecki, et al., 2018).

### Pseudotime

In example embodiments, the invention provides for identifying transcription factors whose overexpression can differentiate stem cells or progenitor cells into target cell types by using single cell sequencing methods. In example embodiments, selecting cells further comprises inferring pseudotime distribution of cells by comparing expression profiles of single cells overexpressing one or more of the transcription factors to those overexpressing controls (e.g., empty vector not expressing a transcription factor or a vector overexpressing a control protein), wherein transcription factors that increase pseudotimes direct differentiation. The methods of the invention can use any trajectory inference (TI) method (see, e.g., Cao J, Spielmann M, Qiu X, et al. The single-cell transcriptional landscape of mammalian organogenesis. Nature. 2019; 566(7745):496-502; Chen H, Albergante L, Hsu J Y, et al. Single-cell trajectories reconstruction, exploration and mapping of omics data with STREAM. Nat Commun. 2019; 10(1):1903; and Van den Berge K, Roux de Bézieux H, Street K, et al. Trajectory-based differential expression analysis for single-cell sequencing data. Nat Commun. 2020; 11(1):1201).

Cellular processes, such as cell differentiation and cell maturation, are dynamic in nature and not always well described by discrete analysis like clustering. Therefore, other methods such as single-cell trajectory inference and pseudotime estimation have emerged. These methods allow to study cellular dynamics, delineate cell developmental lineages, and characterize the transition between different cell states. Briefly, single cells are ordered along deterministic or probabilistic trajectories and a numeric value referred to as pseudotime is assigned to each cell to indicate how far it progresses along a dynamic process of interest. Cell trajectory analysis, also known as pseudo-time series (pseudotime) analysis, uses single cell gene expression to order individual cells at pseudo-time, placing the cells at appropriate trajectory positions corresponding to biological processes, such as cell differentiation, by way of the individual cell's asynchronous biological processes. Most TI methods share a common workflow: dimensionality reduction followed by inference of lineages and pseudotimes in the reduced dimensional space. In that reduced dimensional space, a cell's pseudotime for a given lineage is the distance, along the lineage, between the cell and the origin of the lineage. For cells overexpressing TFs, the origin is defined using cells overexpressing controls.

### Target Cell Types

Target cell types may include, but are not limited to an immune cell, intestinal cell, liver cell, kidney cell, lung cell, brain cell, epithelial cell, endoderm cell, neuron, ectoderm cell, islet cell, acinar cell, hematopoietic cell, hepatocyte, skin/keratinocyte, melanocyte, bone/osteocyte, hair/dermal papilla cell, cartilage/chondrocyte, fat cell/adipocyte, skeletal muscular cell, endothelium cell, cardiac muscle/cardiomyocyte, trophoblast. Target cells may also include progenitor cells associated with target cell types. Markers specific to target cell types are well known in the art.

In certain embodiments, target cell types are neural progenitors. In preferred embodiments, neural progenitors are differentiated to obtain a target cell type that is a neuron, astrocyte and/or oligodendrocyte. In more preferred embodiments, the target cell type is a neuron. In more preferred embodiments, the neuron is a GABAergic neuron. Neurons that produce GABA as their output are called GABAergic neurons, and have chiefly inhibitory action at receptors in the adult vertebrate (Rudy, et al., Three Groups of Interneurons Account for Nearly 100% of Neocortical GABAergic Neurons, Dev Neurobiol. 2011 Jan. 1; 71(1): 45-61). Malfunction of GABAergic neurons has been implicated in a number of diseases ranging from epilepsy to schizophrenia, anxiety disorders and autism. Id.

In certain embodiments, cells differentiated by overexpression of specific transcription factors can be further analyzed. Differentiated target cells can be analyzed for expression of biomarkers specific to the target cells or specific to a phenotype associated with the target cells.

The term “biomarker” is widespread in the art and commonly broadly denotes a biological molecule, more particularly an endogenous biological molecule, and/or a detectable portion thereof, whose qualitative and/or quantitative evaluation in a tested object (e.g., in or on a cell, cell population, tissue, organ, or organism) is predictive or informative with respect to one or more aspects of the tested object's phenotype and/or genotype. The terms “marker” and “biomarker” may be used interchangeably throughout this specification. Biomarkers as intended herein may be nucleic acid-based or peptide-, polypeptide- and/or protein-based. For example, a marker may be comprised of peptide(s), polypeptide(s) and/or protein(s) encoded by a given gene, or of detectable portions thereof. Further, whereas the term “nucleic acid” generally encompasses DNA, RNA and DNA/RNA hybrid molecules, in the context of markers the term may typically refer to heterogeneous nuclear RNA (hnRNA), pre-mRNA, messenger RNA (mRNA), or complementary DNA (cDNA), or detectable portions thereof. Such nucleic acid species are particularly useful as markers, since they contain qualitative and/or quantitative information about the expression of the gene. Particularly preferably, a nucleic acid-based marker may encompass mRNA of a given gene, or cDNA made of the mRNA, or detectable portions thereof. Any such nucleic acid(s), peptide(s), polypeptide(s) and/or protein(s) encoded by or produced from a given gene are encompassed by the term “gene product(s)”.

Preferably, markers as intended herein may be extracellular or cell surface markers, as methods to measure extracellular or cell surface marker(s) need not disturb the integrity of the cell membrane and may not require fixation/permeabilization of the cells.

Unless otherwise apparent from the context, reference herein to any marker, such as a peptide, polypeptide, protein, or nucleic acid, may generally also encompass modified forms of said marker, such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “polypeptide” as used throughout this specification generally encompasses polymeric chains of amino acid residues linked by peptide bonds. Hence, insofar a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally-occurring protein parts that ensue from processing of such full-length proteins.

The reference to any marker, including any peptide, polypeptide, protein, or nucleic acid, corresponds to the marker commonly known under the respective designations in the art. The terms encompass such markers of any organism where found, and particularly of animals, preferably warm-blooded animals, more preferably vertebrates, yet more preferably mammals, including humans and non-human mammals, still more preferably of humans.

The terms particularly encompass such markers, including any peptides, polypeptides, proteins, or nucleic acids, with a native sequence, i.e., ones of which the primary sequence is the same as that of the markers found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to somatic mutations, or post-transcriptional or post-translational modifications. Any such variants or isoforms of markers are intended herein. Accordingly, all sequences of markers found in or derived from nature are considered “native”. The terms encompass the markers when forming a part of a living organism, organ, tissue or cell, when forming a part of a biological sample, as well as when at least partly isolated from such sources. The terms also encompass markers when produced by recombinant or synthetic means.

In certain embodiments, markers, including any peptides, polypeptides, proteins, or nucleic acids, may be human, i.e., their primary sequence may be the same as a corresponding primary sequence of or present in a naturally occurring human markers. Hence, the qualifier “human” in this connection relates to the primary sequence of the respective markers, rather than to their origin or source. For example, such markers may be present in or isolated from samples of human subjects or may be obtained by other means (e.g., by recombinant expression, cell-free transcription or translation, or non-biological nucleic acid or peptide synthesis).

The reference herein to any marker, including any peptide, polypeptide, protein, or nucleic acid, also encompasses fragments thereof. Hence, the reference herein to measuring (or measuring the quantity of) any one marker may encompass measuring the marker and/or measuring one or more fragments thereof.

For example, any marker and/or one or more fragments thereof may be measured collectively, such that the measured quantity corresponds to the sum amounts of the collectively measured species. In another example, any marker and/or one or more fragments thereof may be measured each individually. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

The term “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥ 300, ≥400, ≥500 or ≥600 consecutive amino acids of the corresponding full-length peptide, polypeptide, or protein.

The term “fragment” as used throughout this specification with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥ 70, ≥80, ≥90, ≥100, ≥200, ≥300, ≥400, ≥500 or ≥600 consecutive nucleotides of the corresponding full-length nucleic acid.

Cells such as target cells as disclosed herein may in the context of the present specification be said to “comprise the expression” or conversely to “not express” one or more markers, such as one or more genes or gene products; or be described as “positive” or conversely as “negative” for one or more markers, such as one or more genes or gene products; or be said to “comprise” a defined “gene or gene product signature”.

Such terms are commonplace and well-understood by the skilled person when characterizing cell phenotypes. By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells.

A marker, for example a gene or gene product, for example a peptide, polypeptide, protein, or nucleic acid, or a group of two or more markers, is “detected” or “measured” in a tested object (e.g., in or on a cell, cell population, tissue, organ, or organism) when the presence or absence and/or quantity of said marker or said group of markers is detected or determined in the tested object, preferably substantially to the exclusion of other molecules and analytes, e.g., other genes or gene products.

The terms “increased” or “increase” or “upregulated” or “upregulate” as used herein generally mean an increase by a statically significant amount. For avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “reduced” or “reduce” or “decrease” or “decreased” or “downregulate” or “downregulated” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least 50%, or at least 60%, or at least 70%, or at least 80%, at least 90% or more, up to and including a 100% decrease (i.e., absent level as compared to a reference sample), or any decrease between 10-100% as compared to a reference level, as that.

The terms “quantity”, “amount” and “level” are synonymous and generally well-understood in the art. The terms as used throughout this specification may particularly refer to an absolute quantification of a marker in a tested object (e.g., in or on a cell, cell population, tissue, organ, or organism, e.g., in a biological sample of a subject), or to a relative quantification of a marker in a tested object, i.e., relative to another value such as relative to a reference value, or to a range of values indicating a base-line of the marker. Such values or ranges may be obtained as conventionally known.

An absolute quantity of a marker may be advantageously expressed as weight or as molar amount, or more commonly as a concentration, e.g., weight per volume or mol per volume. A relative quantity of a marker may be advantageously expressed as an increase or decrease or as a fold-increase or fold-decrease relative to said another value, such as relative to a reference value. Performing a relative comparison between first and second variables (e.g., first and second quantities) may but need not require determining first the absolute values of said first and second variables. For example, a measurement method may produce quantifiable readouts (such as, e.g., signal intensities) for said first and second variables, wherein said readouts are a function of the value of said variables, and wherein said readouts may be directly compared to produce a relative value for the first variable vs. the second variable, without the actual need to first convert the readouts to absolute values of the respective variables.

Reference values may be established according to known procedures previously employed for other cell populations, biomarkers and gene or gene product signatures. For example, a reference value may be established in an individual or a population of individuals characterized by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In certain embodiments, the target cells may be detected, quantified, sorted or isolated using a technique selected from the group consisting of flow cytometry, mass cytometry, fluorescence activated cell sorting (FACS), fluorescence microscopy, affinity separation, magnetic cell separation, microfluidic separation, RNA-seq (e.g., bulk or single cell), quantitative PCR, MERFISH (multiplex (in situ) RNA FISH) and combinations thereof. The technique may employ one or more agents capable of specifically binding to one or more gene products expressed or not expressed by the target cells, preferably on the cell surface of the target cells. The one or more agents may be one or more antibodies. Other methods including absorbance assays and colorimetric assays are known in the art and may be used herein.

In other example embodiments, detection of a marker may include immunological assay methods, wherein the ability of an assay to separate, detect and/or quantify a marker (such as, preferably, peptide, polypeptide, or protein) is conferred by specific binding between a separable, detectable and/or quantifiable immunological binding agent (antibody) and the marker. Immunological assay methods include without limitation immunohistochemistry, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (RIA), western blot, etc.

In certain example embodiments, detection of a marker or signature may include biochemical assay methods, including inter alia assays of enzymatic activity, membrane channel activity, substance-binding activity, gene regulatory activity, or cell signaling activity of a marker, e.g., peptide, polypeptide, protein, or nucleic acid.

In other example embodiments, detection of a marker may include mass spectrometry analysis methods. Generally, any mass spectrometric (MS) techniques that are capable of obtaining precise information on the mass of peptides, and preferably also on fragmentation and/or (partial) amino acid sequence of selected peptides (e.g., in tandem mass spectrometry, MS/MS; or in post source decay, TOF MS), may be useful herein for separation, detection and/or quantification of markers (such as, preferably, peptides, polypeptides, or proteins). Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS arrangements, instruments and systems suitable for biomarker peptide analysis may include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; atmospheric pressure photoionization mass spectrometry (APPI-MS); APPI-MS/MS; and APPI-(MS)n. Peptide ion fragmentation in tandem MS (MS/MS) arrangements may be achieved using manners established in the art, such as, e.g., collision induced dissociation (CID). Detection and quantification of markers by mass spectrometry may involve multiple reaction monitoring (MRM), such as described among others by Kuhn et al. 2004 (Proteomics 4: 1175-86). MS peptide analysis methods may be advantageously combined with upstream peptide or protein separation or fractionation methods, such as for example with the chromatographic and other methods.

In other example embodiments, detection of a marker may include chromatography methods. In a one example embodiment, chromatography refers to a process in which a mixture of substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography may be columnar. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immunoaffinity, immobilized metal affinity chromatography, and the like.

In certain embodiments, further techniques for separating, detecting and/or quantifying markers may be used in conjunction with any of the above described detection methods. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In certain examples, such methods may include separating, detecting and/or quantifying markers at the nucleic acid level, more particularly RNA level, e.g., at the level of hnRNA, pre-mRNA, mRNA, or cDNA. Standard quantitative RNA or cDNA measurement tools known in the art may be used. Non-limiting examples include hybridization-based analysis, microarray expression analysis, digital gene expression profiling (DGE), RNA-in-situ hybridization (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, single-cell RNA sequencing (sc-RNA seq), or the like.

The present invention is also directed to signatures and uses thereof. In certain embodiments, a homogenous population of a target cell type (e.g., radial glia) may allow identification of specific signatures (e.g., rare signatures). As used herein a “signature” may encompass any gene or genes, protein or proteins, or epigenetic element(s) whose expression profile or whose occurrence is associated with a specific cell type, subtype, or cell state of a specific cell type or subtype within a population of cells (e.g., radial glia). In certain embodiments, the expression of the target cell signatures is dependent on epigenetic modification of the genes or regulatory elements associated with the genes. Thus, in certain embodiments, use of signature genes includes epigenetic modifications that may be detected or modulated. For ease of discussion, when discussing gene expression, any gene or genes, protein or proteins, or epigenetic element(s) may be substituted. Reference to a gene name throughout the specification encompasses the human gene, mouse gene and all other orthologues as known in the art in other organisms. As used herein, the terms “signature”, “expression profile”, or “expression program” may be used interchangeably. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular target cell or target cell (sub)population if it is upregulated or only present, detected or detectable in that particular target cell or target cell (sub)population, or alternatively is downregulated or only absent, or undetectable in that particular target cell or target cell (sub)population. In this context, a signature consists of one or more differentially expressed genes/proteins or differential epigenetic elements when comparing different cells or cell (sub)populations, including comparing different target cell or target cell (sub)populations, as well as comparing target cell or target cell (sub)populations with non-target cell or non-target cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population or subpopulation level, refer to genes that are differentially expressed in all or substantially all cells of the population or subpopulation (such as at least 80%, preferably at least 90%, such as at least 95% of the individual cells). This allows one to define a particular subpopulation of target cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

In certain embodiments, cells overexpressing transcription factors may be analyzed for the ability to further differentiate (e.g., radial glia can be differentiated to astrocytes, oligodendrocytes and neurons). The cells may be analyzed by analyzing spontaneous or directed differentiation methods. In certain embodiments, cells are analyzed by performing xenografts in immune compromised animal models. In certain embodiments, the cells are analyzed for the ability to repair or regenerate diseased tissue.

### Oncology Screening

In certain embodiments, the barcoded transcription library can be used for a method of pooled screening for transcription factors that enhance or suppress tumor growth. Expression of tumor suppressors have been shown to suppress tumor growth (see, e.g., Wang et al., Restoring expression of wild-type p53 suppresses tumor growth but does not cause tumor regression in mice with a p53 missense mutation. J Clin Invest. 2011 March; 121(3):893-904). In certain embodiments, the method is used to identify therapeutic targets for treating specific cancers. Cancer cell lines for any cancer type may be used. Cancer cell lines may be obtained from a patient. In certain embodiments, the barcoded transcription factor library is introduced to a cancer cell line in vitro, the cells are grown (e.g., 1 to 3 weeks), and the enrichment and depletion of barcodes in the cells is determined as compared to the barcodes present in the original library. In certain embodiments, the barcoded transcription factor library is introduced to a cancer cell line in vitro and transferred to an in vivo model (e.g., nude mice), the cells are grown in vivo (e.g., 1 to 8 weeks), tumor cells are removed (e.g., the tumor), and the enrichment and depletion of barcodes in the cells is determined as compared to the barcodes present in the original library. Barcodes that are enriched represent transcription factors that enhance tumor growth. These transcription factor may be targeted for inhibition to suppress tumor growth. Barcodes that are depleted represent transcription factors that suppress tumor growth. These transcription factors may be overexpressed or activated to suppress tumor growth.

### Combinatorial TF Screening and Prediction

In example embodiments, the genes and gene programs expressed in cells screened by overexpression of single transcription factors is used to identify transcription factor combinations to differentiate stem cells into a target cell type. In example embodiments, single cells overexpressing single transcription factors are used to identify one or more differentially expressed genes as compared to cells not expressing a transcription factor. In one embodiment, a transcription factor atlas as described herein is used. The differentially expressed genes can be used to determine combinations of transcription factors for directing differentiation of stem cells into target cells that more faithfully recapitulate the in vivo target cells. Thus, providing for improved cellular models and therapeutics. In one example embodiment, the average expression of differentially expressed genes for two or more transcription factors are compared to the gene expression of the differentially expressed genes in the target cell. The combination of transcription factors that provide an average expression that most closely recapitulates the expression in the target cell can be used to differentiate stem cells into the target cells. In example embodiments, the average is taken from 2, 3, 4, or more transcription factors, preferably, 2, 3, or 4 transcription factors. In example embodiments, more than 1 gene is averaged, for example, more than 10, 100, 1,000, 5,000, or 10,000 genes. In example embodiments, the genes are part of a gene program, expression program, or pathway as described herein.

In example embodiments, combinations of TFs can be screened using the methods and libraries described herein. For example, a library of 4, 5, 6, 7, 8, 9, 10, 20 or more transcription factors can be introduced to stem cells. In preferred embodiments, the TF library is introduced at high MOI (e.g., greater than 1, 2, 3, 4, 5 or more vectors per cell). In example embodiments, the cells are profiled by single cell RNA-seq. Using the pooled screening methods described herein TF combinations can be identified that are overexpressed by each single cell.

### Use of Target Cells and Transcription Factors

### In Vitro Models

In certain embodiments, the present invention provides methods of generating target cell types in vitro. In vitro models may be obtained by overexpressing transcription factors identified through screening as described herein. In certain embodiments, the methods advantageously produce homogeneous cell types. The methods also provide target cells with reduced labor, time and cost.

In certain embodiments, the in vitro models of the present invention may be used to study development, cell biology and disease. In certain embodiments, the in vitro models of the present invention may be used to screen for drugs capable of modulating the target cells or for determining toxicity of drugs (e.g., toxic to cardiomyocytes). In certain embodiments, the in vitro models of the present invention may be used to identify specific cell states and/or subtypes.

In certain embodiments, the in vitro models of the present invention may be used in perturbation studies. Perturbations may include conditions, substances or agents. Agents may be of physical, chemical, biochemical and/or biological nature. Perturbations may include treatment with a small molecule, protein, RNAi, CRISPR system, TALE system, Zinc finger system, meganuclease, pathogen, allergen, biomolecule, or environmental stress. Such methods may be performed in any manner appropriate for the particular application.

In certain embodiments, the in vitro models are configured for performing perturb-seq. Methods and tools for genome-scale screening of perturbations in single cells using CRISPR have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol. 14 No. 3 DOI: 10.1038/nmeth.4177; Hill et al., On the design of CRISPR-based single cell molecular screens, Nat Methods. 2018 April; 15(4): 271-274; Replogle, et al., “Combinatorial single-cell CRISPR screens by direct guide RNA capture and targeted sequencing” Nat Biotechnol (2020). doi.org/10.1038/s41587-020-0470-y; and International Patent Publication No. WO 2017/075294). In certain embodiments, stem cells are configured for expression of a CRISPR enzyme, such that the cells can be induced to differentiate by overexpressing a transcription factor and barcoded guide sequences can be introduced to the cells.

### Differentiation of Progenitor Cells

In certain embodiments, target cells are further differentiated. In certain embodiments, cells are differentiated by spontaneous differentiation. In certain embodiments, cells are differentiated by directed differentiation.

As used herein the term “spontaneous differentiation” refers to a process where progenitor cells spontaneously differentiate into a target cell and usually involves removal of growth factors from the media. In certain embodiments, the process of spontaneous differentiation can be accelerated by suboptimal culture conditions, such as cultivation to high density for extended periods (4-7 weeks) without replacement of a feeder layer. In certain embodiments, neural progenitor cells obtained by overexpressing transcription factors are spontaneously differentiated into neurons, astrocytes and oligodendrocytes by removal of growth factors from the media (see, e.g., Example 1-2).

As used herein the term “directed differentiation” refers to exposing the stem cells or pluripotent cells to specific signaling pathways modulators and manipulating cell culture conditions (environmental or exogenous) to mimic the natural sequence of developmental decisions to produce a given cell type/tissue. In certain embodiments, pluripotent stem cells (PSCs) are cultured in controlled conditions involving specific substrate or extracellular matrices promoting cell adhesion and differentiation, and defined culture media compositions. A limited number of signaling factors, such as growth factors or small molecules, controlling cell differentiation is applied sequentially or in a combinatorial manner, at varying dosage and exposure time (Cohen D E, Melton D, 2011 “Turning straw into gold: directing cell fate for regenerative medicine”. Nature Reviews Genetics. 12 (4): 243-252). In certain embodiments, radial glia produced using the TF overexpression method as described herein can also be differentiated by directed differentiation into neurons, astrocytes, oligodendrocytes, or organoids.

As used herein, the term “organoid” or “epithelial organoid” refers to a cell cluster or aggregate that resembles an organ, or part of an organ, and possesses cell types relevant to that particular organ. Organoid systems have been described previously, for example, for brain, retinal, stomach, lung, thyroid, small intestine, colon, liver, kidney, pancreas, prostate, mammary gland, fallopian tube, taste buds, salivary glands, and esophagus (see, e.g., Clevers, Modeling Development and Disease with Organoids, Cell. 2016 Jun. 16; 165(7):1586-1597).

In certain embodiments, directed differentiation may include the use of hormones, cytokines, growth factors, mitogens or any other differentiation promoting agents.

In certain embodiments, dual SMAD inhibition (Chambers et al., 2009; Shi et al., 2012a) is used to differentiate RFX4 neural progenitor cells towards CNS cell types, radial glia, and neurons. In certain embodiments, the neurons are GABAergic neurons. Dual SMAD inhibition may include two inhibitors of SMAD signaling. One inhibitor may be a BMP inhibitor. BMP inhibitors include chordin, follistatin, and noggin (Chambers et al., 2009). The two inhibitors may be Noggin and SB431542. SB431542 inhibits the Lefty/Activin/TGFβ pathways by blocking phosphorylation of ALK4, ALK5, ALK7 receptors. Id.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-γ, IL-2, IL-3, IL-4, IL-6, granulocyte-macrophage colony-stimulating factor (GM-CSF), interferon-γ, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-α, interferon-β, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-10, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signaling molecules, such as tumor necrosis factor (TNF) and interferons (e.g., interferon-α, interferon-β, interferon-γ, interferon-λ, or combinations thereof).

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as immunomodulants include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

In certain embodiments, differentiation promoting agents may be used to obtain particular types of target cells. Differentiation promoting agents include anticoagulants, chelating agents, and antibiotics. Examples of such agents may be one or more of the following: vitamins and minerals or derivatives thereof, such as A (retinol), B₃, C (ascorbate), ascorbate 2-phosphate, D such as D₂ or D₃, K, retinoic acid, nicotinamide, zinc or zinc compound, and calcium or calcium compounds; natural or synthetic hormones such as hydrocortisone, and dexamethasone; amino acids or derivatives thereof, such as L-glutamine (L-glu), ethylene glycol tetracetic acid (EGTA), proline, and non-essential amino acids (NEAA); compounds or derivatives thereof, such as β-mercaptoethyl, dibutyl cyclic adenosine monophosphate (db-CAMP), monothioglycerol (MTG), putrescine, dimethyl sulfoxide (DMSO), hypoxanthine, adenine, forskolin, cilostamide, and 3-isobutyl-1-methylxanthine; nucleosides and analogues thereof, such as 5-azacytidine; acids or salts thereof, such as ascorbic acid, pyruvate, okadic acid, linoleic acid, ethylenediaminetetraacetic acid (EDTA), anticoagulant citrate dextrose formula A (ACDA), disodium EDTA, sodium butyrate, and glycerophosphate; antibiotics or drugs, such as G418, gentamycine, Pentoxifylline (1-(5-oxohexyl)-3,7-dimethylxanthine), and indomethacin; and proteins such as tissue plasminogen activator (TPA).

### Transdifferentiation

In certain embodiments, the screening platform and methods of screening are used for identifying transcription factors that drive transdifferentiation of cells into target cell types. As used herein, the terms “transdifferentiation” and “lineage reprogramming” refer to the process by which a committed cell of a first cell lineage is changed into another cell of a different cell type or a process in which one mature somatic cell transforms into another mature somatic cell without undergoing an intermediate pluripotent state or progenitor cell type. In some embodiments, transdifferentiation may be a combination of retrodifferentiation and redifferentiation. A “transdifferentiated cell” is a cell that results from transdifferentiation of a committed cell. For example, a committed cell such as a blood cell or glial cell may be transdifferentiated into a neuron; or a fibroblast may be transdifferentiated into a myocyte. As used herein, “retrodifferentiation” is the process by which a committed cell, i.e., mature, specialized cell, reverts back to a more primitive cell stage. A “retrodifferentiated cell” is a cell that results from retrodifferentiation of a committed cell. As used herein, “redifferentiation” refers to the process by which an uncommitted cell or a retrodifferentiated cell differentiates into a more mature, specialized cell. A “redifferentiated cell” refers to a cell that results from redifferentiation of an uncommitted cell or a retrodifferentiated cell. If a redifferentiated cell is obtained through redifferentiation of a retrodifferentiated cell, the redifferentiated cell may be of the same or different lineage as the committed cell that had undergone retrodifferentiation. For example, a committed cell such as a white blood cell may be retrodifferentiated to form a retrodifferentiated cell such as a pluripotent stem cell, and then the retrodifferentiated cell may be redifferentiated to form a lymphocyte, which is of the same lineage as the white blood cell (committed cell), or redifferentiated to form a neuron, which is of a different lineage than the white blood cell (committed cell).

In certain embodiments, transcription factors are used to transdifferentiate cells of one lineage into a target cell of a different lineage. In certain embodiments, target cell types can be transferred to a subject in need thereof to regenerate a diseased or damaged tissue. One study showed that that islet α-cells can be lineage-traced and reprogrammed by the transcription factors PDX1 and MAFA to produce and secrete insulin in response to glucose that are capable of reversing diabetes in mice (see, e.g., Furuyama, K. et al., 2019 Diabetes relief in mice by glucose-sensing insulin-secreting human α-cells Nature 567, 43-48). Another study showed that functional cardiomyocytes can be directly reprogrammed from differentiated somatic cells using three developmental transcription factors (i.e., Gata4, Mef2c and Tbx5) (see, e.g., Ieda, et al. (2010). “Direct Reprogramming of Fibroblasts into Functional Cardiomyocytes by Defined Factors”. Cell. 142 (3): 375-386. Another study identified that a combination of three factors, Ascl1, Bm2 and Myt11, sufficed to convert mouse embryonic and postnatal fibroblasts into functional neurons in vitro (see, e.g., Vierbuchen, et al., (2010). “Direct conversion of fibroblasts to functional neurons by defined factors”. Nature. 463 (7284): 1035-1041). In certain embodiments, transcription factors that differentiate stem cells into a target cell (e.g., progenitor cell) can be used to transdifferentiate cells of one lineage into a target cell of a different lineage. In certain embodiments, TFs that are expressed in progenitor cells can be used to transdifferentiate cells of one lineage into a target cell of a different lineage (see, e.g., Graf, T.; Enver, T. (2009). “Forcing cells to change lineages”. Nature. 462 (7273): 587-594). In this approach, transcription factors from progenitor cells of the target cell type are transfected into a somatic cell to induce transdifferentiation. Determining the unique set of cellular factors that is needed to be manipulated for each cell conversion is a long and costly process that involves much trial and error. Previous methods required narrowing down factors one by one. As a result, this first step of identifying the key set of cellular factors for cell conversion is the major obstacle researchers face in the field of cell reprogramming. In certain embodiments, the pooled screening methods described herein are used for determining which transcription factors to use.

In certain embodiments, cells can be transdifferentiated to target cells in vivo by targeted modulation of transcription factors or downstream targets. In certain embodiments, the targeted modulation of transcription factors can be used to regenerate, replenish or replace damaged or diseased cells in a subject in need thereof (e.g., heart cells, pancreatic β cells, eye cells, nervous system cells).

In certain embodiments, modulation of one or more of the transcription factors RFX4, NFIB, ASCL1 and PAX6 are used to transdifferentiate glia cells into neurons, astrocytes, or oligodendrocytes. For example, oligodendrocytes may be produced to regenerate the myelin sheath on axons.

In certain embodiments, modulation of one or more of the transcription factors MESP1, EOMES and ESR1 are used to transdifferentiate cardiofibroblasts into cardiomyocytes. For example, cardiomyocytes may be produced to regenerate a damaged heart.

### Cell State Transitions

In certain embodiments, the screening platform and methods of screening are used for identifying transcription factors that modify the cell state or cell state transitions of target cell types. In example embodiments, cell state reflects the fact that cells of a particular type can exhibit variability with regard to one or more features and/or can exist in a variety of different conditions, while retaining the features of their particular cell type and not gaining features that would cause them to be classified as a different cell type. The different states or conditions in which a cell can exist may be characteristic of a particular cell type (e.g., they may involve properties or characteristics exhibited only by that cell type and/or involve functions performed only or primarily by that cell type) or may occur in multiple different cell types. Sometimes a cell state reflects the capability of a cell to respond to a particular stimulus or environmental condition (e.g., whether or not the cell will respond, or the type of response that will be elicited) or is a condition of the cell brought about by a stimulus or environmental condition. Cells in different cell states may be distinguished from one another in a variety of ways. For example, they may express, produce, or secrete one or more different genes, proteins, or other molecules (“markers”), exhibit differences in protein modifications such as phosphorylation, acetylation, etc., or may exhibit differences in appearance. Thus, a cell state may be a condition of the cell in which the cell expresses, produces, or secretes one or more markers, exhibits particular protein modification(s), has a particular appearance, and/or will or will not exhibit one or more biological response(s) to a stimulus or environmental condition.

In example embodiments, a transcription factor or combination of TFs can transition a cell from expressing one cell program to another cell program while the cell type remains the same (e.g., biological program, signature, expression program as described herein). For example, a cell may transition from an “old cell signature” to a “young cell signature” for rejuvenation (e.g., transitioning an “old neuron” to “young neuron”). Another example is enhancing certain cell functions, such as increasing efficiency of T cell killing by transitioning “exhausted T cell signature” to “active or naïve T cell signature.”

Another example of cell state is “activated” state as compared with “resting” or “non-activated” state. Many cell types in the body have the capacity to respond to a stimulus by modifying their state to an activated state. The particular alterations in state may differ depending on the cell type and/or the particular stimulus. A stimulus could be any biological, chemical, or physical agent to which a cell may be exposed.

Another example of cell state reflects the condition of cell (e.g., a muscle cell or adipose cell) as either sensitive or resistant to insulin. Insulin resistant cells exhibit decreased response to circulating insulin; for example, insulin-resistant skeletal muscle cells exhibit markedly reduced insulin-stimulated glucose uptake and a variety of other metabolic abnormalities that distinguish these cells from cells with normal insulin sensitivity.

In an example embodiment, the cell state is an immune cell state. The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4⁺/CD8⁺ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

As used throughout this specification, “immune response” refers to a response by a cell of the immune system, such as a B cell, T cell (CD4⁺ or CD⁸⁺), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4⁺ response or a CD8⁺ response. Such responses by these cells can include, for example, cytotoxicity, proliferation, cytokine or chemokine production, trafficking, or phagocytosis, and can be dependent on the nature of the immune cell undergoing the response.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

Depending on the cytokines used for differentiation, in vitro polarized Th17 cells can either cause severe autoimmune responses upon adoptive transfer (‘pathogenic Th17 cell state’) or have little or no effect in inducing autoimmune disease (‘non-pathogenic cell state’) (Ghoreschi et al., 2010; and Lee et al., 2012 “Induction and molecular signature of pathogenic Th17 cells,” Nature Immunology, vol. 13(10): 991-999). A dynamic regulatory network controls Th17 differentiation (See e.g., Yosef et al., Dynamic regulatory network controlling Th17 cell differentiation, Nature, vol. 496: 461-468 (2013); Wang et al., CD5L/AIM Regulates Lipid Biosynthesis and Restrains Th17 Cell Pathogenicity, Cell Volume 163, Issue 6, p 1413-1427, 3 Dec. 2015; Gaublomme et al., Single-Cell Genomics Unveils Critical Regulators of Th17 Cell Pathogenicity, Cell Volume 163, Issue 6, p 1400-1412, 3 Dec. 2015; and International publication numbers WO2016138488A2, WO2015130968, WO/2012/048265, WO/2014/145631 and WO/2014/134351, the contents of which are hereby incorporated by reference in their entirety).

Markers specific for the cell state can be determined for each TF as described previously (e.g., activated, quiescent, exhausted cell state markers). Markers can be determined, for example, by scRNA-seq (e.g., entire programs), flow FISH, reporters, etc.

### Therapeutic Compositions and Uses

In certain embodiment, the cells produced according to the present invention are used for treatment, to model a disease, or to screen for therapeutic agents. In certain embodiments, target cells obtained according to the methods described herein may be used for the treatment of a subject in need thereof. In certain embodiments, target cells transdifferentiated according to the methods described herein may be used for the treatment of a subject in need thereof. In certain embodiments, target cells are transferred to a subject to repair, regenerate, replace or replenish a target tissue or cell type. In certain embodiments, transcription factors or agents capable of modulating expression or activity of the transcription factors or downstream pathways are introduced in vivo to generate target cells. In certain embodiments, the TFs or agents are introduced to a specific target region requiring the target cells.

As used herein, a “subject” is a vertebrate, including any member of the class mammalia. As used herein, a “mammal” refers to any mammal including but not limited to human, mouse, rat, sheep, monkey, goat, rabbit, hamster, horse, cow or pig.

In certain embodiments, a cell-based therapeutic includes engraftment of the cells of the present invention. As used herein, the term “engraft” or “engraftment” refers to the process of cell incorporation into a tissue of interest in vivo through contact with existing cells of the tissue.

In certain embodiments, the cell based therapy may comprise adoptive cell transfer (ACT). As used herein adoptive cell transfer and adoptive cell therapy are used interchangeably. In certain embodiments, the target cells differentiated according to the methods described herein may be transferred to a subject in need thereof. If possible, use of autologous cells helps the recipient by minimizing GVHD issues. In certain embodiments, autologous stem cells are harvested from a subject and the cells are modulated to overexpress the transcription factor(s) to differentiate the stem cells into target cells.

In certain embodiments, the target cells are used as a cell-based therapy to treat a subject suffering from a disease. In certain embodiments, the disease may be treated by infusion of target cell types (see, e.g., US Patent Publication No. 20110091433A1 and Table 2 of application). In certain embodiments, a disease may be treated by inducing target cells in vivo. Target cells may be induced by expressing transcription factors at a specific site of the disease. Transcription factors may be provided to specific cells at a location of disease. In certain embodiments, mRNA is provided. In certain embodiments, transdifferentiation of target cells is performed in vivo.

### Diseases

In certain embodiment, the cells produced according to the present invention are used for treatment, to model a disease, or to screen for therapeutic agents. The disease may be selected from the group consisting of bone marrow failure, hematological conditions, aplastic anemia, beta-thalassemia, diabetes, neuron disease, motor neuron disease, Parkinson's disease, spinal cord injury, muscular dystrophy, kidney disease, liver disease, multiple sclerosis, congestive heart failure, head trauma, lung disease, psoriasis, liver cirrhosis, vision loss, cystic fibrosis, hepatitis C virus, human immunodeficiency virus, inflammatory bowel disease (IBD), and any disorder associated with tissue degeneration.

In certain embodiments, the neuron disease may be a disease where GABAergic neurons are implicated. In certain embodiments, the disease may be autism, schizophrenia, epilepsy, dementia, Alzheimer's disease, or anxiety disorders (e.g., depression) (Rudy, et al., Three Groups of Interneurons Account for Nearly 100% of Neocortical GABAergic Neurons, Dev Neurobiol. 2011 Jan. 1; 71(1): 45-61; Xu and Wong, GABAergic Inhibitory Neurons as Therapeutic Targets for Cognitive Impairment in Schizophrenia, Acta Pharmacol Sin. 2018 May; 39(5): 733-753; Fogaça and Duman, Cortical GABAergic Dysfunction in Stress and Depression: New Insights for Therapeutic Interventions, Front Cell Neurosci. 2019; 13: 87; Choi et al., Pathology of nNOS expressing GABAergic neurons in mouse model of Alzheimer's disease, Neuroscience. 2018 Aug. 1; 384: 41-53; Treiman, GABAergic Mechanisms in Epilepsy, Epilepsia. 2001; 42 Suppl 3:8-12; and Coghlan et al., GABA System Dysfunction in Autism and Related Disorders: From Synapse to Symptoms, Neurosci Biobehav Rev. 2012 October; 36(9): 2044-2055).

Aplastic anemia is a rare but fatal bone marrow disorder, marked by pancytopaenia and hypocellular bone marrow (Young et al. Blood 2006, 108: 2509-2519). The disorder may be caused by an immune-mediated pathophysiology with activated type I cytotoxic T cells expressing Th1 cytokine, especially y-interferon targeted towards the haematopoietic stem cell compartment, leading to bone marrow failure and hence anhaematoposis (Bacigalupo et al. Hematology 2007, 23-28). The majority of aplastic anaemia patients can be treated with stem cell transplantation obtained from HLA-matched siblings (Locasciulli et al. Haematologica. 2007; 92:11-18.).

Thalassaemia is an inherited autosomal recessive blood disease marked by a reduced synthesis rate of one of the globin chains that make up hemoglobin. Thus, there is an underproduction of normal globin proteins, often due to mutations in regulatory genes, which results in formation of abnormal hemoglobin molecules, causing anemia. Different types of thalassemia include alpha thalassemia, beta thalassemia, and delta thalassemia, which affect production of the alpha globin, beta globin, and delta globin, respectively.

Diabetes is a syndrome resulting in abnormally high blood sugar levels (hyperglycemia). Diabetes refers to a group of diseases that lead to high blood glucose levels due to defects in either insulin secretion or insulin action in the body. Diabetes is typically separated into two types: type 1 diabetes, marked by a diminished production of insulin, or type 2 diabetes, marked by a resistance to the effects of insulin. Both types lead to hyperglycemia, which largely causes the symptoms generally associated with diabetes, e.g., excessive urine production, resulting compensatory thirst and increased fluid intake, blurred vision, unexplained weight loss, lethargy, and changes in energy metabolism.

Motor neuron diseases refer to a group of neurological disorders that affect motor neurons. Such diseases include amyotrophic lateral sclerosis (ALS), primary lateral sclerosis (PLS), and progressive muscular atrophy (PMA). ALS is marked by degeneration of both the upper and lower motor neurons, which ceases messages to the muscles and results in their weakening and eventual atrophy. PLS is a rare motor neuron disease affecting upper motor neurons only, which causes difficulties with balance, weakness and stiffness in legs, spasticity, and speech problems. PMA is a subtype of ALS that affects only the lower motor neurons, which can cause muscular atrophy, fasciculations, and weakness.

Parkinson's disease (PD) is a neurodegenerative disorder marked by the loss of the nigrostriatal pathway, resulting from degeneration of dopaminergic neurons within the substantia nigra. The cause of PD is not known, but is associated with the progressive death of dopaminergic (tyrosine hydroxylase (TH) positive) mesencephalic neurons, inducing motor impairment. Hence, PD is characterized by muscle rigidity, tremor, bradykinesia, and potentially akinesia.

Spinal cord injury is characterized by damage to the spinal cord and, in particular, the nerve fibers, resulting in impairment of part or all muscles or nerves below the injury site. Such damage may occur through trauma to the spine that fractures, dislocates, crushes, or compresses one or more of the vertebrae, or through nontraumatic injuries caused by arthritis, cancer, inflammation, or disk degeneration.

Muscular dystrophy (MD) refers to a set of hereditary muscle diseases that weaken skeletal muscles. MD may be characterized by progressive muscle weakness, defects in muscle proteins, muscle cell apoptosis, and tissue atrophy. There are over 100 diseases which exhibit MD characteristics, although nine diseases in particular—Duchenne, Becker, limb girdle, congenital, facioscapulohumeral, myotonic, oculopharyngeal, distal, and Emery-Dreifuss—are classified as MD.

Kidney disease refers to conditions that damage the kidneys and decrease their ability to function, which includes removal of wastes and excess water from the blood, regulation of electrolytes, blood pressure, acid-base balance, and reabsorption of glucose and amino acids. The two main causes of kidney disease are diabetes and high blood pressure, although other causes include glomerulonephritis, lupus, and malformations and obstructions in the kidney.

Multiple sclerosis is an autoimmune condition in which the immune system attacks the central nervous system, leading to demyelination. MS affects the ability of nerve cells in the brain and spinal cord to communicate with each other, as the body's own immune system attacks and damages the myelin which enwraps the neuron axons. When myelin is lost, the axons can no longer effectively conduct signals. This can lead to various neurological symptoms which usually progresses into physical and cognitive disability. In certain embodiments, target cells may include oligodendrocytes.

Congestive heart failure refers to a condition in which the heart cannot pump enough blood to the body's other organs. This condition can result from coronary artery disease, scar tissue on the heart cause by myocardial infarction, high blood pressure, heart valve disease, heart defects, and heart valve infection. Treatment programs typically consist of rest, proper diet, modified daily activities, and drugs such as angiotensin-converting enzyme (ACE) inhibitors, beta blockers, digitalis, diuretics, vasodilators. However, the treatment program will not reverse the damage or condition of the heart.

Hepatitis C is an infectious disease in the liver, caused by hepatitis C virus. Hepatitis C can progress to scarring (fibrosis) and advanced scarring (cirrhosis). Cirrhosis can lead to liver failure and other complications such as liver cancer.

Head trauma refers to an injury of the head that may or may not cause injury to the brain. Common causes of head trauma include traffic accidents, home and occupational accidents, falls, and assaults. Various types of problems may result from head trauma, including skull fracture, lacerations of the scalp, subdural hematoma (bleeding below the dura mater), epidural hematoma (bleeding between the dura mater and the skull), cerebral contusion (brain bruise), concussion (temporary loss of function due to trauma), coma, or even death.

Lung disease is a broad term for diseases of the respiratory system, which includes the lung, pleural cavity, bronchial tubes, trachea, upper respiratory tract, and nerves and muscles for breathing. Examples of lung diseases include obstructive lung diseases, in which the bronchial tubes become narrowed; restrictive or fibrotic lung diseases, in which the lung loses compliance and causes incomplete lung expansion and increased lung stiffness; respiratory tract infections, which can be caused by the common cold or pneumonia; respiratory tumors, such as those caused by cancer; pleural cavity diseases; and pulmonary vascular diseases, which affect pulmonary circulation.

### Pharmaceutical Compositions

Target cells of the present invention may be combined with various components to produce compositions of the invention. The compositions may be combined with one or more pharmaceutically acceptable carriers or diluents to produce a pharmaceutical composition (which may be for human or animal use). Suitable carriers and diluents include, but are not limited to, isotonic saline solutions, for example phosphate-buffered saline. The composition of the invention may be administered by direct injection. The composition may be formulated for parenteral, intramuscular, intravenous, subcutaneous, intraocular, oral, transdermal administration, or injection into the spinal fluid.

Compositions comprising target cells may be delivered by injection or implantation. Cells may be delivered in suspension or embedded in a support matrix such as natural and/or synthetic biodegradable matrices. Natural matrices include, but are not limited to, collagen matrices. Synthetic biodegradable matrices include, but are not limited to, polyanhydrides and polylactic acid. These matrices may provide support for fragile cells in vivo.

The compositions may also comprise the target cells of the present invention, and at least one pharmaceutically acceptable excipient, carrier, or vehicle.

Delivery may also be by controlled delivery, i.e., delivered over a period of time which may be from several minutes to several hours or days. Delivery may be systemic (for example by intravenous injection) or directed to a particular site of interest. Cells may be introduced in vivo using liposomal transfer.

Target cells may be administered in doses of from 1×10⁵ to 1×10⁷ cells per kg. For example a 70 kg patient may be administered 1.4×10⁶ cells for reconstitution of tissues. The dosages may be any combination of the target cells listed in this application.

### Genetic Modifying Agents

In certain embodiments, the one or more modulating agents (e.g., for overexpressing transcription factors, silencing transcription factors or tagging cells with a detectable marker) may be a genetic modifying agent. The genetic modifying agent may comprise a CRISPR system, a zinc finger nuclease system, a TALEN, a meganuclease, or RNAi.

### CRISPR

In certain embodiments, a CRISPR system is used to enhance expression or activity of transcription factors. In certain embodiments, the transcription factor expression or activity is enhanced temporarily, such that the enhancement is not permanent. In certain embodiments, expression of the transcription from its endogenous gene is enhanced (e.g., by directing an activator to the gene).

In certain embodiments, modification of transcription factor mRNA by a Cas13-deaminase system can be used to modulate transcription factor activity in order to generate target cells (see, e.g., International Patent Publication No. WO 2019/084062). In certain embodiments, the modification silences ubiquitination, methylation, acetylation, succinylation, glycosylation, O-GlcNAc, O-linked glycosylation, iodination, nitrosylation, sulfation, carboxyglutamation, phosphorylation, or a combination thereof. In some embodiments, the modification increases a half-life of a target TF. In certain embodiments, the transcription activity is enhanced by modifying a phosphorylation site on the transcription factor (see, e.g., Hunter and Karin, 1992, The regulation of Transcription by Phosphorylation. Cell, Vol. 70, 375-387; and Whitmarsh and Davis, 2000, Regulation of transcription factor function by phosphorylation. CMLS, Cell. Mol. Life Sci. 57: 1172).

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

In certain embodiments, a CRISPR system is used to enhance expression or activity of transcription factors (e.g., RFX4, NFIB, ASCL1, PAX6). In certain embodiments, the transcription factor expression or activity is enhanced temporarily, such that the enhancement is not permanent. In certain embodiments, expression of the transcription from its endogenous gene is enhanced (e.g., by directing an activator to the gene). In certain embodiments, genes are targeted for downregulation. In certain embodiments, genes are targeted for editing.

In certain embodiments, modification of transcription factor mRNA by a Cas13-deaminase system can be used to modulate transcription factor activity in order to generate target cells (see, e.g., International Patent Publication No. WO 2019/084062). In certain embodiments, the modification silences ubiquitination, methylation, acetylation, succinylation, glycosylation, O-GlcNAc, O-linked glycosylation, iodination, nitrosylation, sulfation, carboxyglutamation, phosphorylation, or a combination thereof. In some embodiments, the modification increases a half-life of a target TF. In certain embodiments, the transcription activity is enhanced by modifying a phosphorylation site on the transcription factor (see, e.g., Hunter and Karin, 1992, The regulation of Transcription by Phosphorylation. Cell, Vol. 70, 375-387; and Whitmarsh and Davis, 2000, Regulation of transcription factor function by phosphorylation. CMLS, Cell. Mol. Life Sci. 57: 1172).

### Class 1 CRISPR-Cas Systems

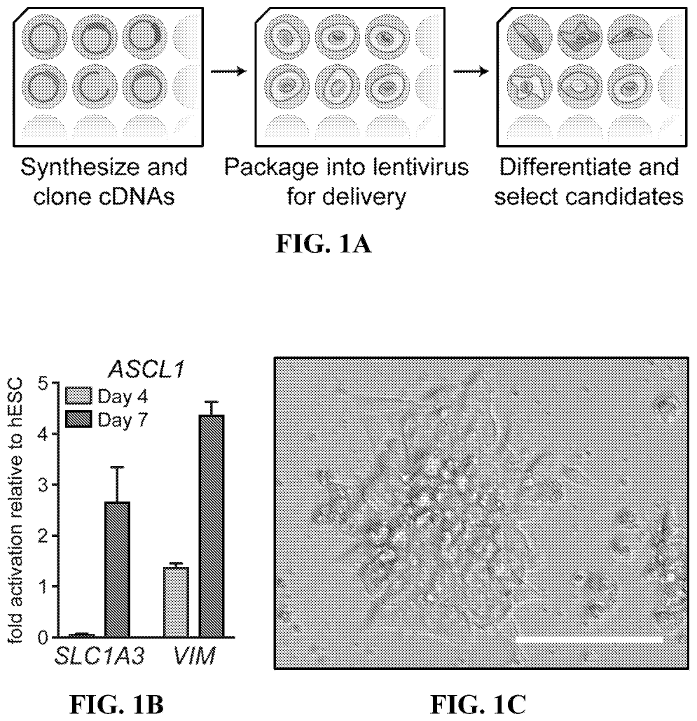

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n 5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits, e.g., Cas 5, Cas6, and/or Cas7. RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

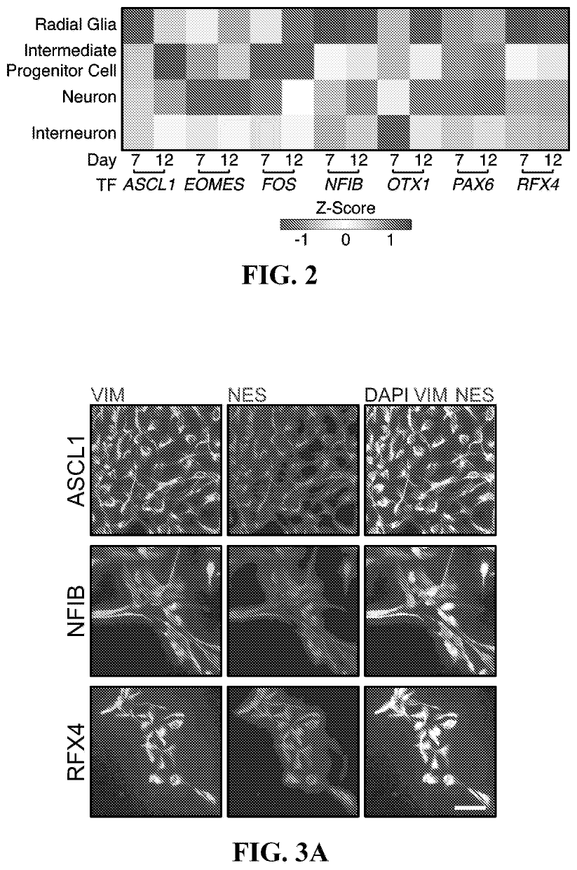

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas 5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-FI(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g. Cas9) contain two nuclear domains that are each responsible for the cleavage of one strand of the target DNA, with the HNH nuclease inserted inside the Ruv-C like nuclease domain sequence. The Type V systems (e.g. Cas12) only contain a RuvC-like nuclease domain that cleaves both strands. Type VI (Cas13) are unrelated to the effectors of type II and V systems, contain two HEPN domains and target RNA. Cas13 proteins also display collateral activity that is triggered by target recognition. Some Type V systems have also been found to possess this collateral activity two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g., VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g. Zetche et al., 2015. Nat. Biotechnol. 33(2): 139-142, the compositions and techniques of which can be used in and/or adapted for use with the present invention. Split CRISPR-Cas proteins are set forth herein and in documents incorporated herein by reference in further detail herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### Base Editing

In some embodiments, a polynucleotide of the present invention described elsewhere herein (e.g., RFX4, NFIB, ASCL1, PAX6) can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018.Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g., A:T to G:C). Richter et al. 2020. Nature Biotechnology. doi.org/10.1038/s41587-020-0453-z.

Other Example Type V base editing systems are described in International Patent Publication Nos. WO 2018/213708 and WO 2018/213726, and International Patent Application Nos. PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editing

In some embodiments, a polynucleotide of the present invention described elsewhere herein (e.g. RFX4, NFIB, ASCL1, PAX6) can be modified using a prime editing system (See e.g. Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRISPR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

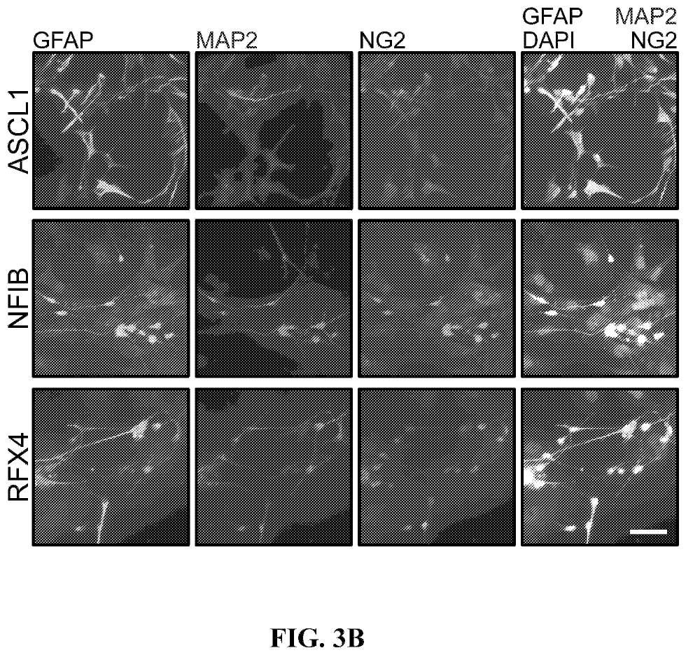

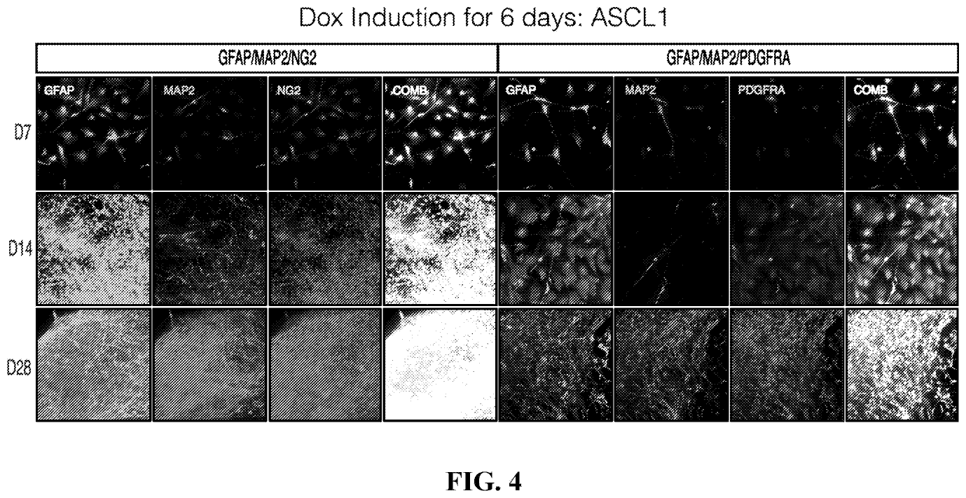

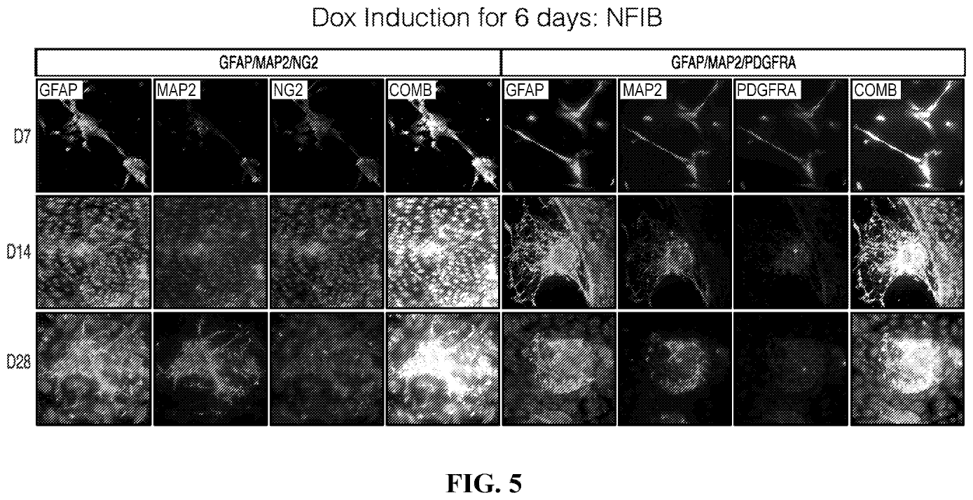

In some embodiments, the prime editing system can be a PEI system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CAST Systems

In some embodiments, a polynucleotide of the present invention described elsewhere herein (e.g., RFX4, NFIB, ASCL1, PAX6) can be modified using a CRISPR-Associated Transposase (CAST) System, such as any of those described in PCT/US2019/066835. In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class 1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and International Patent Application No. PCT/US2019/066835, which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target polynucleotide can be a polynucleotide or a part of a polynucleotide to which a part of the guide sequence is designed to have complementarity to and to which the effector function mediated by the complex comprising the CRISPR effector protein and a guide molecule is to be directed to. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g. Gleditzsch et al. 2019. RNA Biology. 16(4):504-517. Table 15 below shows several Cas polypeptides and the PAM sequence they recognize.

TABLE 15
Example PAM Sequences

| | |
|---|---|
| Cas Protein | PAM Sequence |
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g. target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35) z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 10788)
M D P I R S R T P S P A R E L L S G P Q
P D G V Q P T A D R G V S P P A G G P L
D G L P A R R T M S R T R L P S P P A P
S P A F S A D S F S D L L R Q F D P S L
F N T S L F D S L P P F G A H H T E A A
T G E W D E V Q S G L R A A D A P P P T
M R V A V T A A R P P R A K P A P R R R
A A Q P S D A S P A A Q V D L R T L G Y
S Q Q Q Q E K I K P K V R S T V A Q H H
E A L V G H G F T H A H I V A L S Q H P
A A L G T V A V K Y Q D M I A A L P E A
T H E A I V G V G K Q W S G A R A L E A
L L T V A G E L R G P P L Q L D T G Q L
L K I A K R G G V T A V E A V H A W R N
A L T G A P L N

An exemplary amino acid sequence of a C-terminal capping region is:

(SEQ ID NO: 10789)
R P A L E S I V A Q L S R P D P A L A A
L T N D H L V A L A C L G G R P A L D A
V K K G L P H A P A L I K R T N R R I P
E R T S H R V A D H A Q V V R V L G F F
Q C H S H P A Q A F D D A M T Q F G M S
R H G L L Q L F R R V G V T E L E A R S
G T L P P A S Q R W D R I L Q A S G M K
R A K P S P T S T Q T P D Q A S L H A F
A D S L E R D L D A P S P M H E G D Q T
R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 10790) or PKKKRKVEAS (SEQ ID NO: 10791); the NLS from nucleoplasmin (e.g., the nucleoplasmin bipartite NLS with the sequence KRPAATKKAGQAKKKK (SEQ ID NO: 10792)); the c-myc NLS having the amino acid sequence PAAKRVKLD (SEQ ID NO: 10793) or RQRRNELKRSP (SEQ ID NO: 10794); the hRNPA1 M9 NLS having the sequence NQSSNFGPMKGGNFGGRSSGPYGGGGQYFAKPRNQGGY (SEQ ID NO: 10795); the sequence RMRIZFKNKGKDTAELRRRRVEVSVELRKAKKDEQILKRRNV (SEQ ID NO: 10796) of the IBB domain from importin-alpha; the sequences VSRKRPRP (SEQ ID NO: 10797) and PPKKARED (SEQ ID NO: 10798) of the myoma T protein; the sequence PQPKKKPL (SEQ ID NO: 10799) of human p53; the sequence SALIKKKKKMAP (SEQ ID NO: 10800) of mouse c-abl IV; the sequences DRLRR (SEQ ID NO: 10801) and PKQKKRK (SEQ ID NO: 10802) of the influenza virus NS1; the sequence RKLKKKIKKL (SEQ ID NO: 10803) of the Hepatitis virus delta antigen; the sequence REKKKFLKRR (SEQ ID NO; 10804) of the mouse Mx1 protein; the sequence KRKGDEVDGVDEVAKKKSKK (SEQ ID NO: 10805) of the human poly(ADP-ribose) polymerase; and the sequence RKCLQAGMNLEARKTKK (SEQ ID NO: 10806) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 1 1, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Delivery

The programmable nucleic acid modifying agents and other modulating agents, or components thereof, or nucleic acid molecules thereof (including, for instance HDR template), or nucleic acid molecules encoding or providing components thereof, may be delivered by a delivery system herein described.

Vector delivery, e.g., plasmid, viral delivery: the modulating agents, can be delivered using any suitable vector, e.g., plasmid or viral vectors, such as adeno associated virus (AAV), lentivirus, adenovirus or other viral vector types, or combinations thereof. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

In certain embodiments, mRNA encoding the transcription factors are delivered to a subject in need thereof. In certain embodiments, the mRNA is modified mRNA (see, e.g., U.S. Pat. No. 9,428,535 B2)

In certain embodiments, proteins, mRNA or cells are administered via targeted injection (e.g., the tissue to be repaired), intravenous, infusion, or other delivery methods. Such delivery may be either via a single dose, or multiple doses. One skilled in the art understands that the actual dosage to be delivered herein may vary greatly depending upon a variety of factors, such as the target cell, or tissue, the general condition of the subject to be treated, the degree of modification sought, the administration route, the administration mode, the type of modification sought, etc.

In certain embodiment, transcription factors are expressed in target tissue cells temporarily. In certain embodiments, the time of transcription factor expression or enhancement is only the time required to differentiate or transdifferentiate cells into target cells. In certain embodiments, transcription factors are expressed or enhanced for 1 to 14 days, preferably, about 2 days. In certain embodiments, the means of delivery does not result in integration of a sequence encoding transcription factors in the genome of target cells.

The invention is further described in the following examples, which do not limit the scope of the invention described in the claims.

### EXAMPLES

### Example 1—Identification of Transcription Factors that Differentiate hESCs into Radial Glia

Radial glia are neural progenitors of the developing mammalian brain capable of generating neurons, astrocytes, and oligodendrocytes. The two most established methods for producing neural progenitors, embryoid body formation and dual SMAD inhibition, are not high-throughput and produce non-homogenous neural progenitor populations (Chambers S M, et al., Highly efficient neural conversion of human ES and iPS cells by dual inhibition of SMAD signaling. Nat Biotechnol. 2009; 27(3):275-80; and Pankratz M T, et al., Directed neural differentiation of human embryonic stem cells via an obligated primitive anterior stage. Stem Cells. 2007; 25(6):1511-20). Applicants developed a stepwise method for differentiating hESCs into neural progenitors. Although previous studies have shown that overexpression of the TFs ASCL1 and PAX6 can drive differentiation of embryonic stem cells into neural progenitors and neurons, the TFs that direct human radial glia differentiation remain unknown (Chanda S, et al., Generation of induced neuronal cells by the single reprogramming factor ASCL1. Stem Cell Reports. 2014; 3(2):282-96; and Zhang X, et al., Pax6 is a human neuroectoderm cell fate determinant. Cell Stem Cell. 2010; 7(1):90-100). Applicants individually overexpressed candidate TFs that are specifically expressed in radial glia based on available RNA-sequencing (RNA-seq) datasets, and selected those that generate cells expressing radial glia-specific marker genes and presenting associated morphology. Identification of novel TFs that direct radial glia differentiation can enable better understanding of neural development and provide positive controls for establishing a TF screening platform.

To establish a system for TF-directed differentiation, Applicants compared two overexpression methods, cDNA and CRISPR activation (Konermann S, et al., Genome-scale transcriptional activation by an engineered CRISPR-Cas9 complex. Nature. 2015; 517(7536):583-8), to upregulate known TFs that direct differentiation of hESCs to neurons, NEUROD1 and NEUROG2, in the HUES66 hESC line (Zhang Y, et al., 2013). Applicants chose the HUES66 line because of its ability to generate brain organoids efficiently and maintain karyotype stability (Quadrato G, et al., Cell diversity and network dynamics in photosensitive human brain organoids. Nature. 2017; 545(7652):48-53). Applicants found that in this system only cDNA overexpression successfully and efficiently differentiated hESCs into neurons by immunostaining for MAP2, a neuronal marker (specifically, the TF ORF without UTR as described further herein). Based on the results of the comparison, Applicants used cDNA to overexpress TFs individually in a targeted arrayed screen to identify those that could differentiate hESCs into radial glia (FIG. 1a). Applicants selected a set of 73 TFs shown to be specifically expressed in radial glia or neural progenitors in 6 published RNA-seq datasets (Camp J G, et al., Human cerebral organoids recapitulate gene expression programs of fetal neocortex development. Proc Natl Acad Sci USA. 2015; 112(51):15672-7; Johnson M B, et al., Single-cell analysis reveals transcriptional heterogeneity of neural progenitors in human cortex. Nat Neurosci. 2015; 18(5):637-46; Pollen A A, et al., Molecular identity of human outer radial glia during cortical development. Cell. 2015; 163(1):55-67; Thomsen E R, et al., Fixed single-cell transcriptomic characterization of human radial glial diversity. Nat Methods. 2016; 13(1):87-93; Wu J Q, et al., Dynamic transcriptomes during neural differentiation of human embryonic stem cells revealed by short, long, and paired-end sequencing. Proc Natl Acad Sci USA. 2010; 107(11):5254-9; and Zhang Y, et al., Purification and Characterization of Progenitor and Mature Human Astrocytes Reveals Transcriptional and Functional Differences with Mouse. Neuron. 2016; 89(1):37-53). For each TF, Applicants included isoforms that comprised >25% of the expressed transcript, resulting in a total of 90 TF isoforms (Table 1). Applicants chose to synthesize the targeted TF library to avoid potential sequence errors commonly found in existing cDNA libraries, and cloned the library into a vector with a constitutive EF1a promoter. Applicants included a V5 epitope tag and unique 24-nucleotide DNA barcode on each TF to facilitate downstream assessment of protein expression and TF abundance in the cell population respectively (SEQ ID NO: 1-90). Applicants packaged the targeted library into lentivirus for delivery into hESCs and screened the targeted library in an arrayed format, where hESCs in each well of a 96-well plate express a different TF (FIG. 1a). The barcode is transcribed but not translated (i.e., because it is not part of the ORF). The barcode is lentivirally integrated with the cDNA in the genomic DNA. Applicants PCR amplify the barcode from the genomic DNA to identify which cDNA constructs were integrated. At 4 and 7 days after transduction, Applicants evaluated the TFs using imaging for radial glia-like morphology and qPCR for two radial glia marker genes, SLC1A3 and VIM, identified in published RNA-seq datasets (Id). Applicants identified 7 candidate TFs: ASCL1, EOMES, FOS, NFIB, OTX1, PAX6, and RFX4 (FIG. 1b, c).

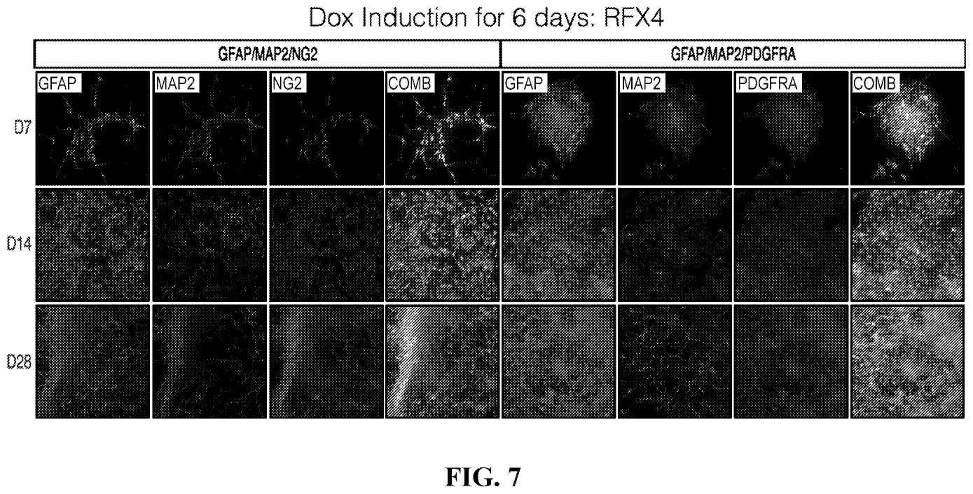

Applicants next evaluated the fidelity of radial glia differentiated from each candidate. First, Applicants performed RNA-seq on radial glia derived from overexpressing each candidate for 7 and 12 days. Gene signature analysis of the RNA-seq data suggested similarities (e.g., EOMES and RFX4) and differences (e.g., NFIB and ASCL1) in the transcriptomes between the candidates. To determine how closely the differentiated radial glia resembled their in vivo counterparts, Applicants computationally generated gene expression signatures based on the 1,000 most differentially expressed genes compared to the GFP overexpression control and quantified enrichment of these signatures in human fetal radial glia and other neural cell types from the Pollen et al. dataset (FIG. 2) (Pollen A A, et al., 2015; and Barbie D A, et al., Systematic RNA interference reveals that oncogenic KRAS-driven cancers require TBK1. Nature. 2009; 462(7269): 108-12). Applicants found that candidates NFIB and RFX4 produced radial glia that were most similar to radial glia in vivo. Second, Applicants immunostained for radial glia markers NES and VIM, and found that all of the radial glia differentiated by these candidates expressed these markers (FIG. 3a). Finally, Applicants spontaneously differentiated the radial glia to determine if they could produce neurons, astrocytes, and oligodendrocytes. Applicants cloned the candidates into a different vector under a dox-inducible promoter and induced expression of the candidates for 5, 7, and 12 days and then withdrew growth factors EGF and bFGF from the media (which maintain the progenitor state) and allowed cells to differentiate for 1, 2, and 4 weeks. Applicants immunostained for markers identifying neurons (MAP2), astrocytes (GFAP), and oligodendrocyte precursors (NG2 and PDGFRA). Similar to neural development in vivo, Applicants observed that neurogenesis occurred before gliogenesis. By 4 weeks of differentiation, radial glia differentiated from 3 of the 7 candidates (FOS, NFIB, and OTX1) produced only neurons and 3 (ASCL1, PAX6, and RFX4) produced both neurons and astrocytes (FIG. 3b). Applicants, further show that the 4 candidates (ASCL1, NFIB, PAX6, and RFX4) differentiate into both neurons and astrocytes by week 4 (FIGS. 4-7). Transcription factors were induced with doxycycline for 6 days and cells were stained at the indicated time points.

### Discussion of Methods for Selection and Characterization of TFs Driving Optimal Radial Glia Differentiation

Applicants can continue to validate the candidate TFs. Applicants have already identified and selected the most promising TFs for further characterization to understand their role in radial glia differentiation. In particular, because some of the candidates did not produce neurons until after 4 weeks of differentiation, Applicants can spontaneously differentiate radial glia derived by candidate TF overexpression for a total of 6-8 weeks to observe additional astrocytes and oligodendrocytes. Applicants can immunostain the cells that have been differentiated for 6 and 8 weeks to determine which candidates generate radial glia that can differentiate into all 3 cell types at this time point. After pinpointing the ideal TF induction and differentiation timeline, Applicants can perform single-cell RNA-seq on the cells spontaneously differentiated from the top 4 candidates to more precisely characterize the types of differentiated cells. Due to the morphology of neural cells and difficulty in dissociating single neural cell types, single nuclei can be isolated from neural cells and sequenced as previously described (see e.g., WO/2017/164936). Applicants can compare the anatomical location of the cell types that the differentiated cells correspond to in vivo to the TF expression pattern in the human brain using the Allen Human Brain Atlas (Sunkin S M, et al., Allen Brain Atlas: an integrated spatio-temporal portal for exploring the central nervous system. Nucleic Acids Res. 2013; 41(Database issue):D996-D1008). To better understand the regulatory pathways through which the TFs drive differentiation, Applicants can also perform chromatin immunoprecipitation followed by sequencing (ChIP-seq) using the epitope tag (e.g., V5) on the TF cDNA constructs and identify target genes for the top 4 candidates. Applicants can integrate differentially expressed genes and TF target genes from the RNA-seq and ChIP-seq results respectively to better understand potential pathway similarities and differences between the top 4 TFs. Finally, Applicants can combine 2 or 3 of the top 4 candidates and assess any potential synergistic improvement in radial glia fidelity using RNA-seq and spontaneous differentiation.

Given the data described herein, Applicants expect to find several candidate TFs whose overexpression can differentiate hESCs into radial glia that closely resemble primary cells. Applicants can also uncover multiple candidate TFs that each produce different subtypes of radial glia. Some of these candidates might upregulate the radial glia marker genes without exhibiting other properties associated with radial glia, such as ability to differentiate into different neural cell types. Since the candidate TFs likely have different downstream gene targets, the radial glia produced can have different transcriptome signatures and spontaneously differentiate into varying proportions of different downstream neural cell types. Applicants expect that the types of downstream cell types identified by single-nuclei RNA-seq can correlate with the expression pattern of the TF in the human brain.

A number of directed differentiation protocols require overexpression of two or more TFs for successful cell type conversion. It is possible that one TF can be insufficient for generating radial glia that can maintain multipotency and spontaneously differentiate into neurons, astrocytes, and oligodendrocytes. In this case, Applicants can select 5-10 candidates that produce cell types with transcriptome signatures that are most similar to human fetal radial glia and overexpress different combinations of these candidates. Applicants can also combine the top 5-10 TFs that are most specifically and highly expressed in radial glia based on available RNA-seq datasets (Camp J G, et al., 2015; Johnson M B, et al., 2015; Pollen A A, et al., 2015; Thomsen E R, et al., 2016; Wu J Q, et al., 2010; and Zhang Y, et al., 2016).

### Example 2—Arrayed TF Screen for iNP Differentiation

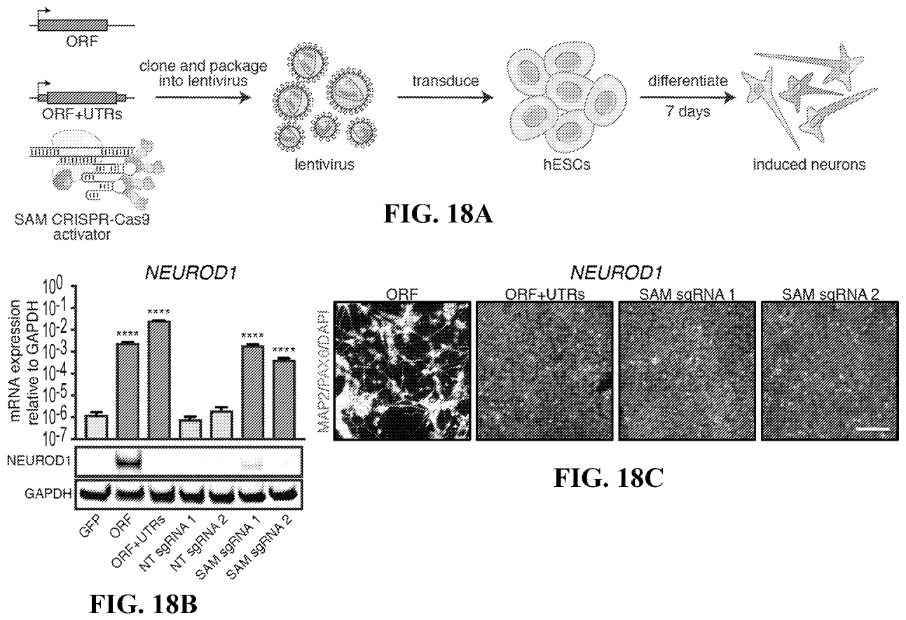

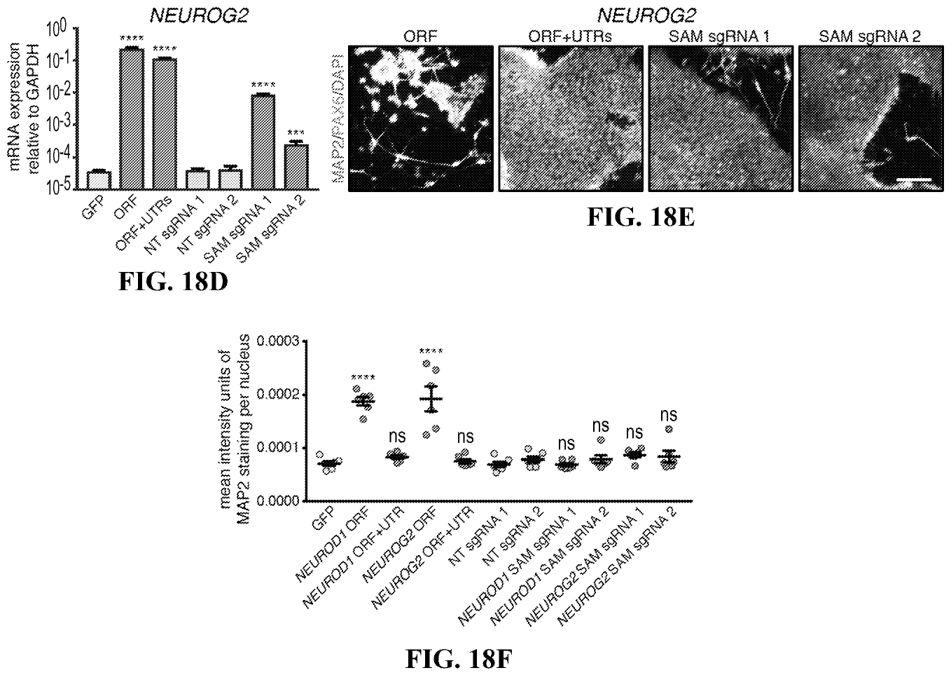

As described in example 1, Applicants compared two methods for overexpressing TFs to direct differentiation, ORF (open reading frame, cDNA) and synergistic activation mediators (SAM) CRISPR-Cas9 activation¹⁶. Applicants used these methods to stably upregulate NEUROD1 or NEUROG2, two TFs that have been previously shown to induce neuronal differentiation, in the HUES66 hESC line (FIG. 18a)¹². For both TFs, Applicants found that expression of the TF ORF effectively induced neuronal differentiation (FIG. 18b-f). However, overexpression of the TFs using the ORF with endogenous UTRs or SAM CRISPR-Cas9 activator did not efficiently differentiate hESCs into neurons despite robust transcriptional upregulation, potentially due to endogenous post-transcriptional regulatory mechanisms that limit protein expression (FIG. 18b-f). The results suggest that cell fate pathways are tightly regulated and that using the most artificial overexpression method, TF ORF, would be advantageous for cellular engineering.

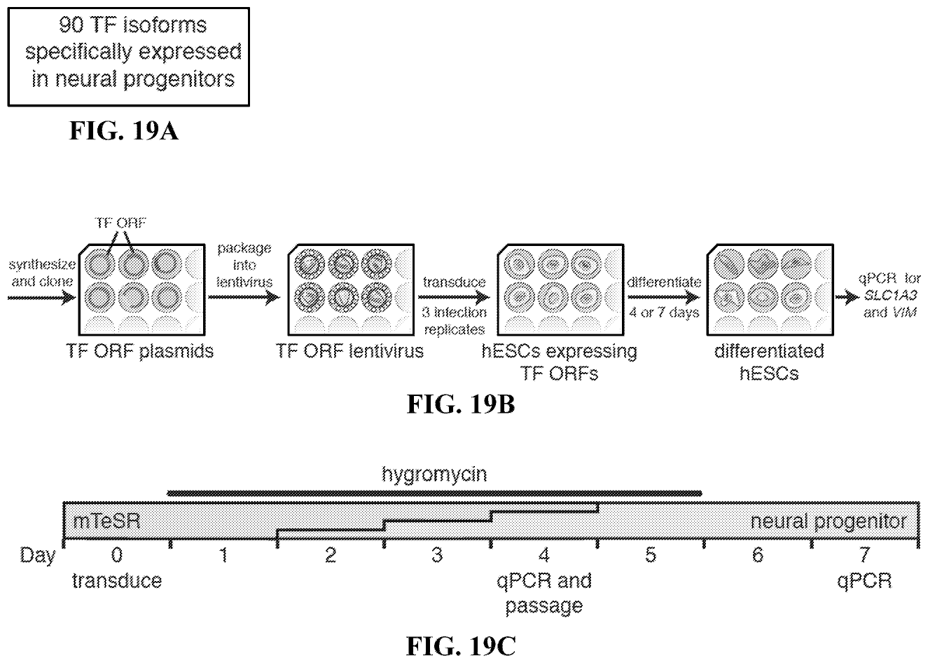

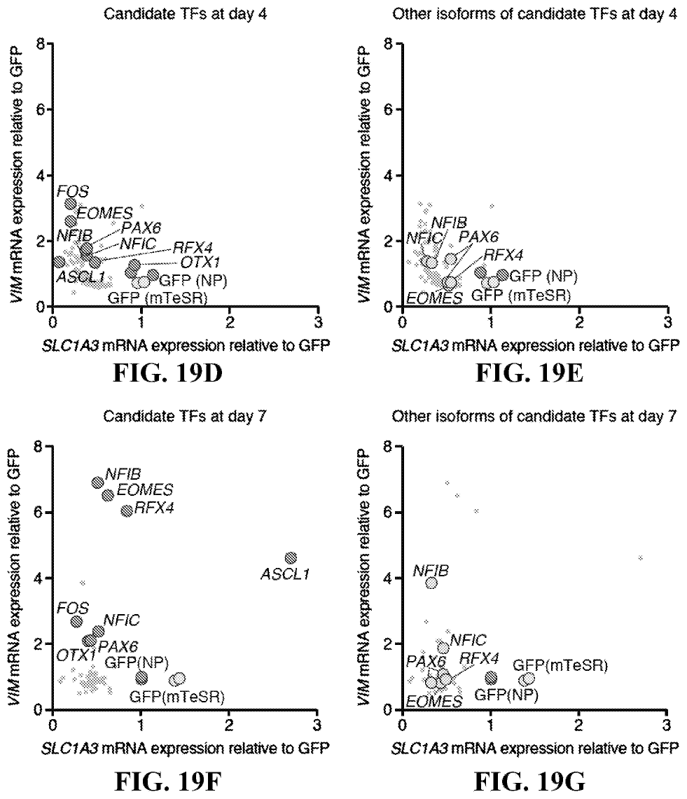

Based on the results of the comparison, Applicants used TF ORF overexpression to screen for TFs that could differentiate hESCs into iNPs first in an arrayed format to identify optimal parameters and candidate TFs that could guide the development of pooled TF screens (FIG. 19a,b). To select a subset of TFs for the arrayed screen, Applicants examined eight RNA-seq datasets¹⁷⁻²⁴ that were available at the time and identified 70 TFs that were shown to be specifically expressed in NPs. For each TF, Applicants included isoforms that comprised >25% of the expressed transcript, resulting in a total of 90 TF isoforms (FIG. 19a and Table 1). Applicants synthesized the TF isoforms and packaged each TF individually into lentivirus for delivery into hESCs in an arrayed format (FIG. 19b). During the screen, Applicants incrementally shifted the stem cell culture media to NP media (FIG. 19c) and measured expression of two NP marker genes selected using published RNA-seq datasets, SLC1A3 and VIM, at 4 and 7 days after transduction (FIG. 19b)¹⁷⁻²⁴. The arrayed TF screen identified eight candidate TFs whose isoforms ranked in the top 10% for SLC1A3 and VIM upregulation in the screen (FIG. 19d-g; Table 1).

### Example 3—Development of a Pooled TF Screening Platform

Pooled screens are less expensive and time-intensive than arrayed screens because they do not require individually preparing each perturbation (e.g., overexpression of TFs) in the library. Pooled screening involves transducing pooled lentiviral libraries at a low multiplicity of infection (MOI) to ensure that most cells only receive one stably integrated construct. At the end of the screen, deep sequencing of DNA barcodes contained in the constructs integrated in the bulk genomic DNA can be used to identify changes in the construct distribution resulting from the applied screening selection pressure. In certain embodiments, cells having characteristic markers for the cell type of interest (e.g., radial glia) are sorted and the DNA barcodes corresponding to TFs are determined, thus identifying TFs required for differentiation into the cell type of interest.

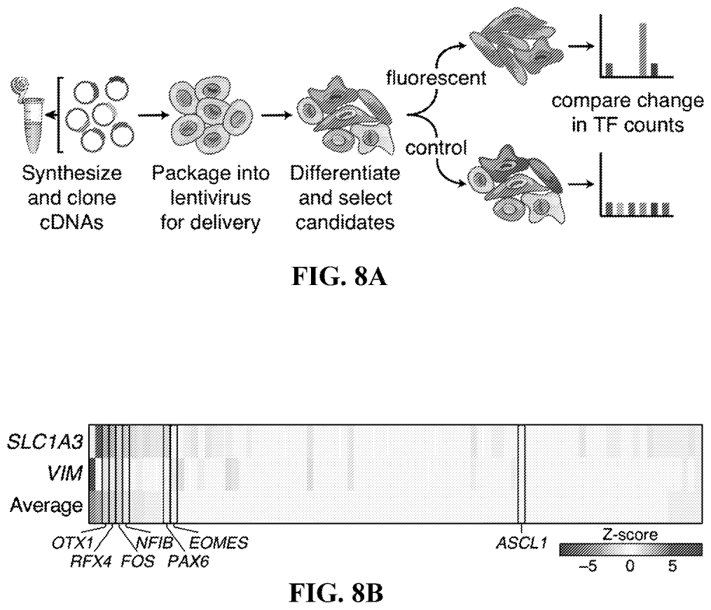

Applicants provide a generalizable TF screening platform based on pooled screening for further identification of regulators driving cellular differentiation (FIG. 8a). Applicants can develop the pooled screen based on the findings differentiating hESCs into radial glia. The pooled screening platform further comprises engineered hESC reporter lines that fluoresce upon differentiation into radial glia by genetically tagging radial glia marker genes with GFP. The pooled screening platform provides a more cost-effective, versatile, and reliable approach compared to antibody staining. In addition, the use of reporter lines for marker genes found through RNA-seq of target cell types increases the versatility of the platform; for any cell type of interest, one can collect RNA-seq data, identify marker genes, and screen for TFs that upregulate the marker genes. Applicants can overexpress pooled TF libraries in the hESC reporter lines, and select for candidates using flow cytometry followed by deep sequencing of the barcodes associated with the cDNAs (FIG. 8a). Applicants can validate the pooled screening approach by pooling the 90 TFs from Examples 1-2 and performing a pooled screen with this targeted TF library. To develop a generalizable platform for differentiating hESCs into any desired cell type, Applicants can scale up the pooled screen first with an available >1300 TF library from the Broad Genomics Perturbations Platform (GPP) and then with a synthesized >3500 TF library consisting of all annotated TFs. The genome-scale TF library can be a valuable resource for constructing a directed differentiation cell atlas that can be helpful for the scientific community.

Applicants have engineered two different HUES66 hESC reporter lines that express the fluorescent protein EGFP upon upregulation of an endogenous radial glia marker gene, either VIM or SLC1A3. Screening in two different marker gene reporter lines can more specifically pinpoint which TFs direct radial glia differentiation rather than upregulate one gene that may also be expressed in other cell types. For each marker gene, Applicants used CRISPR-Cas9 to precisely edit the endogenous locus such that the EGFP is expressed under the same promoter as the marker gene, followed by a ribosomal skipping site P2A and the marker gene (Cong L, et al., Multiplex genome engineering using CRISPR/Cas systems. Science. 2013; 339(6121):819-23; and Mali P, et al., RNA-guided human genome engineering via Cas9. Science. 2013; 339(6121):823-6). Applicants chose to insert EGFP at the N-terminus of the proteins because its location was consistent across the isoforms. The P2A ribosomal skipping site separates the EGFP and marker gene proteins and prevents the EGFP insertion from potentially interfering with protein folding of the endogenous gene. For each marker gene, Applicants generated three clonal hESC lines to reduce the possibility that candidate TFs identified only have an effect in a particular clonal line. Applicants evaluated the ability of the reporter lines to fluoresce upon marker gene upregulation by targeting CRISPR activators to the marker gene promoter as well as by overexpressing a candidate TF from Example 1 to differentiate the hESCs into radial glia (Konermann S, et al., 2015). In both cases, Applicants detected EGFP fluorescence in both marker lines by imaging. For TF overexpression, Applicants also observed morphological changes consistent with radial glia differentiation.

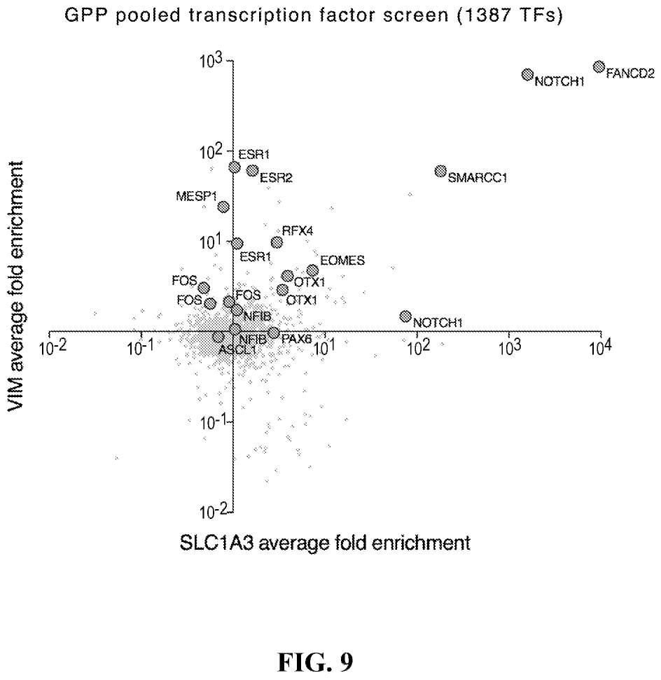

Applicants validated the pooled screening system by pooling the targeted 90 TF library in Examples 1-2 and performing a targeted pooled screen (FIG. 8a). Applicants amplified and packaged the pooled library into lentiviral vectors and transduced each hESC reporter line at MOI<0.3. After 7 days, Applicants used flow cytometry to isolate live cells expressing fluorescent EGFP, indicating upregulation of the radial glia marker gene, and live cells with the lowest 15% fluorescence for baseline TF distribution. Applicants isolated genomic DNA from each population, PCR amplified the DNA barcodes associated with the TFs, and deep sequenced the barcodes to identify TFs that were more enriched in the fluorescent population compared to control in the VIM and SLC1A3 reporter cell lines (FIG. 8b). Applicants found that the candidates identified in Example 1 were significantly enriched in the pooled screens. Six of 7 TFs were in the top 15 candidates. ASCL1 was not enriched in the fluorescent population in the pooled screens, potentially because ASCL1-driven differentiation relies on early formation of neural rosettes (FIG. 1c), or radial arrangements of neural stem cells, which are less likely to form in a pooled screen because nearby cells can be overexpressing different TFs rather than ASCL1. FIG. 9 is a scatterplot of the 1,387 TF screening results, showing that the 7 TF candidates (ASCL1, EOMES, FOS, NFIB, OTX1, PAX6, and RFX4) are enriched and also show additional candidates for differentiating stem cells into radial glia (FANCD2, NOTCH1, SMARCC1, ESR2, ESR1, and MESP1).

### Development of a Versatile Genome-Scale TF Screen

To scale up the pooled TF screen to include all annotated TF isoforms, Applicants can use the >1,300 TF library from the Broad GPP and then synthesize a >3,500 genome-scale TF library that includes all annotated TFs (see, e.g., Table 3). The Broad GPP library is a convenient intermediate because it is readily available at a lower cost. Applicants added the candidates identified in Examples 1-2 to the Broad GPP library as positive controls. Applicants amplified the pooled Broad GPP library and verified even distribution of the TFs with deep sequencing. Applicants can package the Broad GPP library into lentivirus for transducing the hESC radial glia reporter lines. As in the targeted pooled screen, Applicants can isolate the fluorescent and control cell populations and deep sequence the barcodes to compare the TF distribution between the two populations. Applicants can evaluate the results of the Broad GPP library using the candidates identified in Examples 1-2. If the TF screen using the Broad GPP library is successful, Applicants can synthesize the complete >3,500 genome-scale TF library and screen for radial glia differentiation using the genome-scale library.

### Validation of Novel TFs

Applicants can validate any additional TFs identified in the pooled screens using the arrayed methods described in Examples 1-2. If any of the candidate TFs produce radial glia that are comparable with the top 3 candidates identified in Examples 1-2, Applicants can combine the TF(s) from the pooled screens with those from the arrayed screens to potentially improve radial glia fidelity.

### Discussion

By starting with a targeted pooled library and incrementally scaling up to a genome-scale library using known positive controls, Applicants can establish a generalizable TF screening platform. As Applicants increase the TF library size, Applicants expect that the proportion of fluorescent cells in the screening population can decrease. Applicants can adjust the screening parameters, such as increasing flow cytometry time and number of PCR cycles for barcode amplification, to detect the rarer positive population. Performing the pooled screening platform with the genome-scale TF library may provide additional novel TFs that can drive radial glia differentiation.

As shown in Examples 1-2, it is possible that radial glia differentiation can require upregulation of multiple TFs. To screen for combinations of TFs, Applicants can transduce the TF libraries at high MOI such that each cell potentially overexpresses multiple TFs. Applicants can validate the candidates most enriched for radial glia marker gene expression both individually and combinatorically. Multiple barcodes in single cells can be determined by any single cell sequencing method described herein.

Since current neural progenitor differentiation protocols often require formation of neural rosettes, it is possible that pooled screening cannot recover some candidates found in the arrayed screen in Examples 1-2. Applicants can recover these candidates by constructing an inducible TF library (e.g., dox inducible), transducing the library at low cell density, allowing the cells to multiply in small colonies, and then inducing TF overexpression.

Compared to short hairpin RNAs and guide RNAs, cDNAs contain longer variable sequences, which can increase the skew in the distribution of pooled cDNA libraries. If the pooled cDNA libraries are significantly more skewed, Applicants can increase the screening coverage such that more cells are expressing each cDNA.

### Example 4—Development of a Pooled TF Screening Platform Using Flow-FISH

Applicants have further developed a pooled transcription factor screening platform that does not require generating clonal cell lines that express a marker gene. Applicants have used Flow FISH to read out transcription factor screens. The method provides for detecting marker genes for indicating differentiation of target cells using gene specific probes and sorting the cells. In certain embodiments, multiple markers are used to increase specificity. Selecting for multiple reporter genes at the same time can narrow down target cell types because usually one gene is not specific enough depending on the target cell type. Additionally, the assay is versatile in that reporter genes can be added or changed by applying different probes. Flow FISH combines FISH to fluorescently label mRNA of reporter genes and flow cytometry (see, e.g., Arrigucci et al., FISH-Flow, a protocol for the concurrent detection of mRNA and protein in single cells using fluorescence in situ hybridization and flow cytometry, Nat Protoc. 2017 June; 12(6):1245-1260. doi:10.1038/nprot.2017.039). Specifically, Applicants fluorescently label mRNA of reporter genes, select for target cell types by flow cytometry, and then amplify TF barcodes to identify TFs enriched in the target cells. In certain embodiments, the marker genes are selected, such that they are specifically expressed only in the target cell. In this way, false positive selection or background is avoided. The assay is also optimized to remove background fluorescence and to select for true positive cells.

Applicants used the 90 TF library to screen for TFs that differentiate into radial glia by combining both SLC1A3 and VIM probes for those reporter genes (Table 4). The data shows that Applicants were able to selectively enrich for TFs that were identified in the arrayed and reporter gene screens to differentiate radial glia described in Examples 1-3.

### Example 5—Identification of Candidate TFs Using the Pooled TF Screening Platform

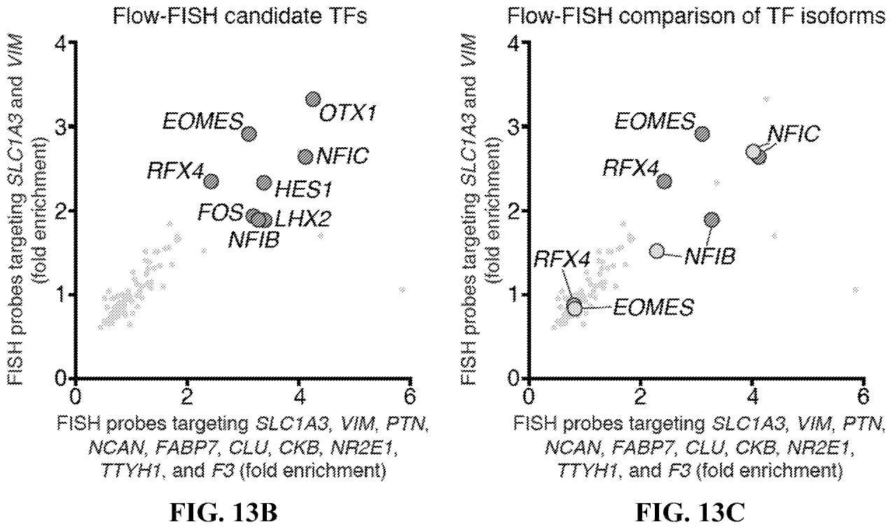

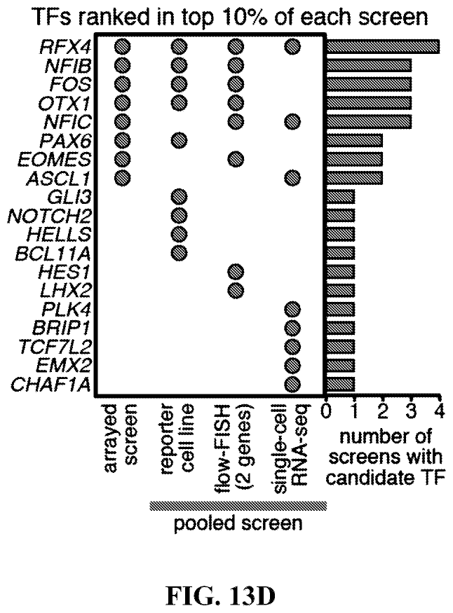

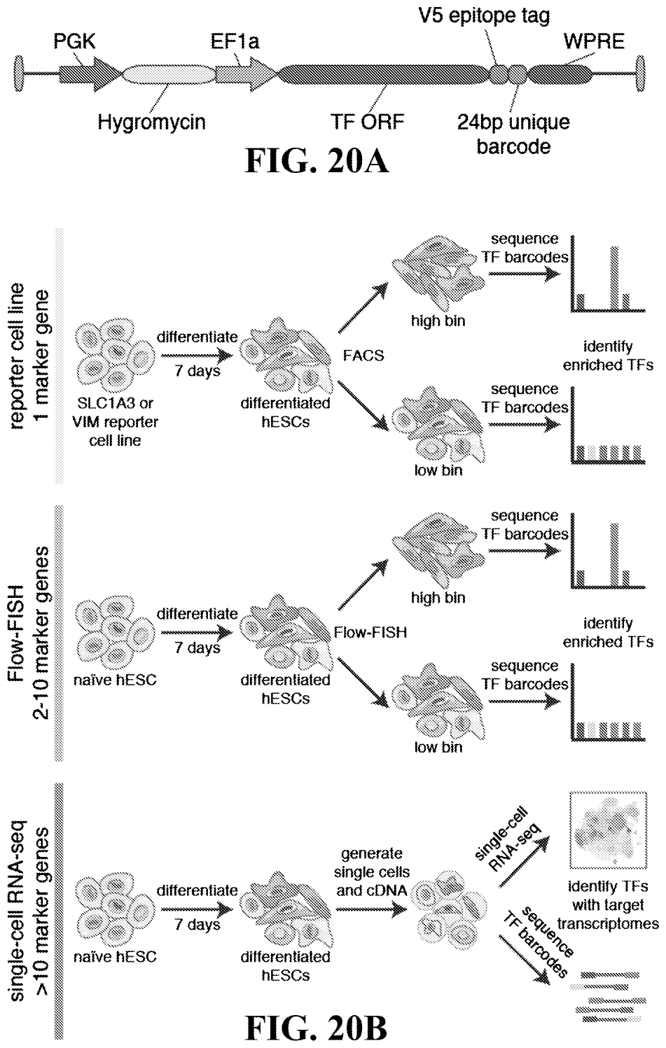

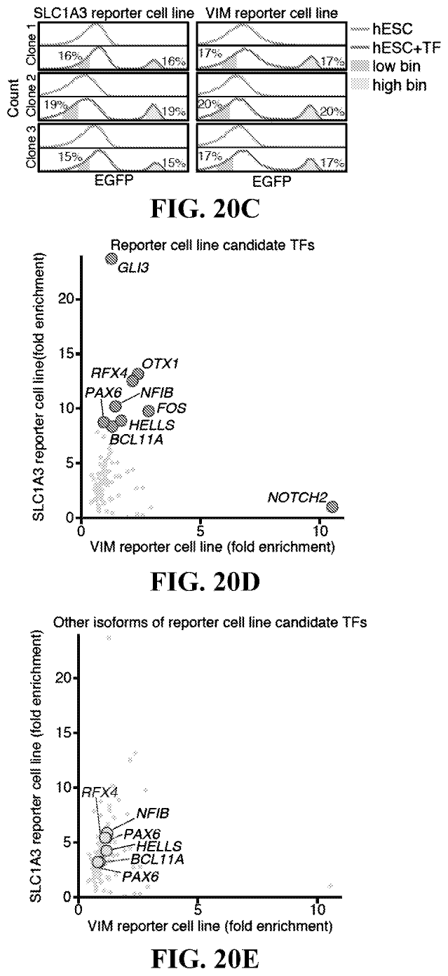

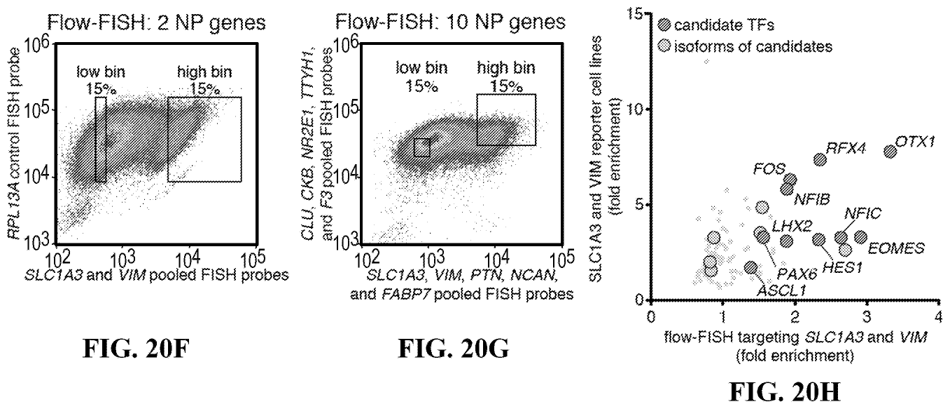

Having optimized parameters and identified candidate TFs in the arrayed screen, Applicants generated a pooled TF screening approach, as described herein. The pooled screening platform is less expensive and laborious than arrayed screening, making it more high-throughput. Applicants simplified TF identification in pooled screens by pairing a unique DNA barcode with each of the 90 TF ORF isoforms synthesized for the arrayed screen (FIG. 20a; Table 1). Applicants pooled the barcoded TFs and packaged the TFs into a pooled lentiviral library for delivery (FIG. 13a). To determine the ideal strategy for selecting TFs that drive iNP differentiation, Applicants explored three different methods that can simultaneously assay different numbers of marker genes to select for target cell types: reporter cell line (1 gene), flow-FISH (up to 10 genes), and single-cell RNA-seq (scRNA-seq; up to ˜2,000 genes; FIG. 13a and FIG. 20b).

For the reporter cell line method, Applicants generated clonal reporter cell lines with EGFP inserted downstream of an endogenous NP marker gene, either SLC1A3 or VIM as described. Applicants transduced the SLCIA3 or VIM reporter cell line with the pooled TF library, differentiated the cells for 7 days, and sorted for high and low EGFP-expressing cells (FIG. 13a and FIG. 20b, c). Deep sequencing of the TF barcodes in each population identified nine candidate TFs that were ranked in the top 10% for enrichment in the high EGFP-expressing cell population, indicating upregulation of SLC1A3 or VIM (FIG. 20d, e and Table 1). Five of the nine candidate TFs were identified in the arrayed screen (FIG. 20d, e and Table 1).

For the flow-FISH method, Applicants transduced hESCs with the pooled TF library, differentiated the cells for 7 days, and labeled 2 or 10 NP marker gene transcripts using pooled FISH probes (FIG. 13a and FIG. 20b). By pooling the FISH probes, Applicants could sort for cells expressing high or low levels of 2-10 marker genes at the same time (FIG. 20f, g). Similar to the reporter cell line method, Applicants deep sequenced the TF barcodes and identified eight candidate TFs whose isoforms ranked in the top 10% for enrichment in cells expressing higher levels of marker genes (FIG. 13b, c and Table 1). Applicants found that for some TFs, such as EOMES and RFX4, the choice of TF isoform can produce very different differentiation results (FIG. 13c). Six of the eight candidate TFs from the flow-FISH screen overlapped with those from the arrayed screen (FIG. 20d, e and Table 1).

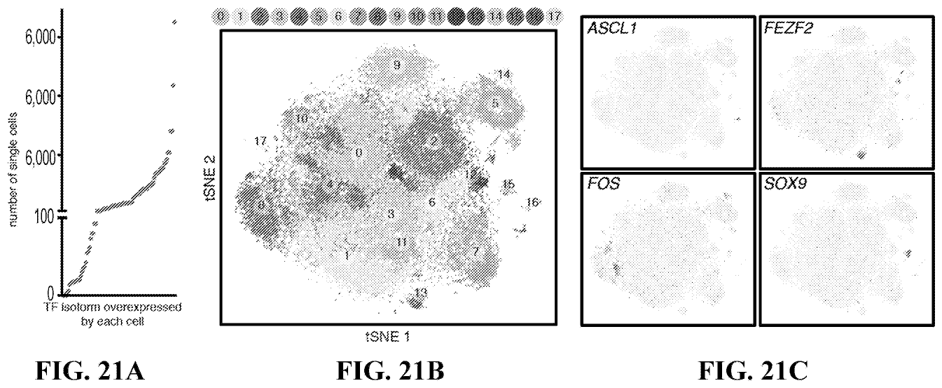

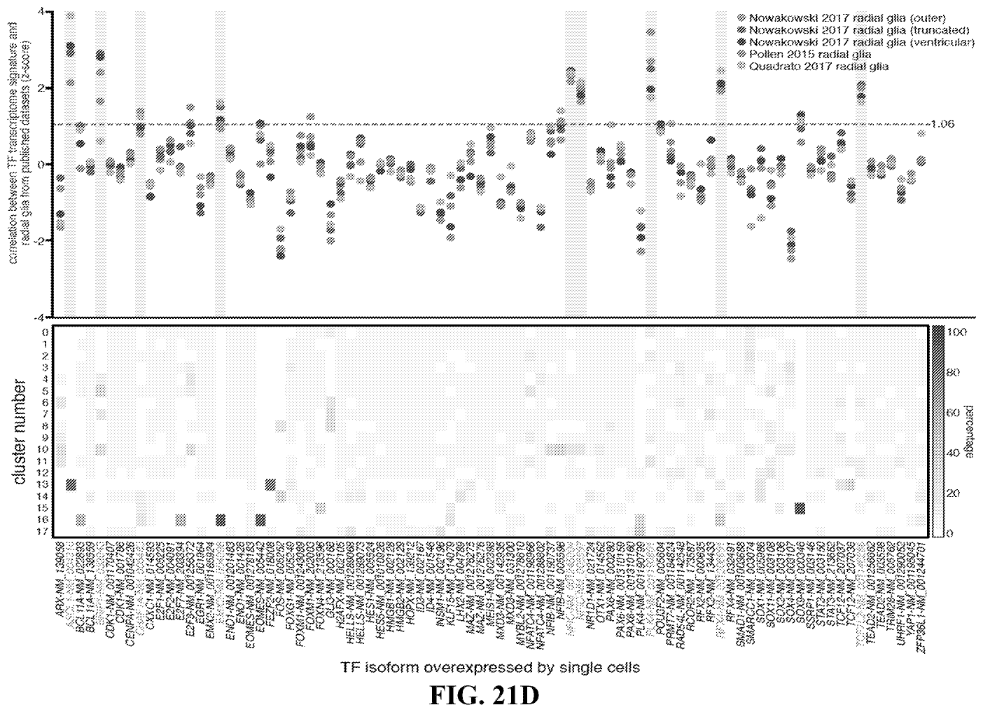

For the scRNA-seq method, Applicants transduced hESCs with the pooled TF library, differentiated the cells for 7 days, and performed scRNA-seq to profile 59,640 single cells (FIG. 13a and FIG. 20b). In the barcoded TF ORF vector design, the TF barcode is expressed in the TF mRNA, which is captured by scRNA-seq and can be mapped to cell barcodes (FIG. 20a). After assigning TFs to cells, Applicants found that the number of cells that had each TF overexpressed was very skewed, with the top 10% of TFs having 92 times more cells than the bottom 10% of TFs, potentially due to TF-dependent effects on cell death and proliferation (FIG. 21a). Cluster analysis of the scRNA-seq results suggested that overexpression of several TFs, for instance ASCL1 and FEZF2, generated distinct transcriptome signatures that clustered together, while overexpression of most TFs did not produce distinct transcriptome signatures (FIG. 21b-d). By correlating the TF transcriptome signatures with those of radial glia from published datasets^{20,25,26}, which represent NPs in the developing cortex, Applicants identified eight candidate TFs whose isoforms ranked in the top 10% for highest correlation (FIG. 21d and Table 1). Three of the eight candidate TFs were candidates identified in the arrayed screen, potentially because scRNA-seq samples provide expression of more genes (FIG. 21d and Table 1).

Overall, the arrayed and pooled screens nominated overlapping sets of candidate TFs for iNP differentiation (FIG. 13d, and Table 1). Out of the pooled screening methods, flow-FISH identified the highest number (6 out of 8) of candidate TFs that overlapped with other screens (FIG. 13d and FIG. 20h). Flow-FISH is also more versatile than reporter cell lines and more accessible than the scRNA-seq, suggesting that it may be the ideal screening method for other cell types.

### Example 6—Validation of Candidate TFs

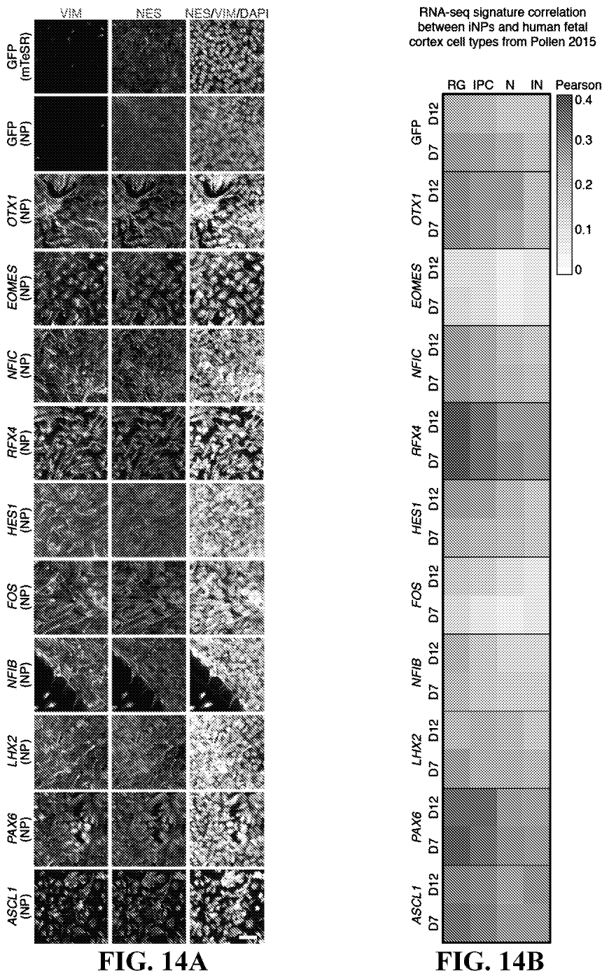

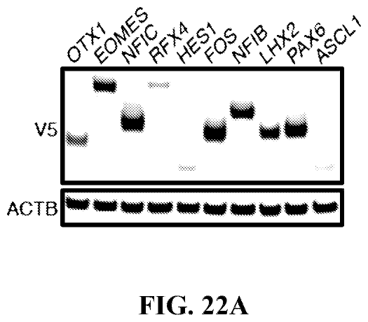

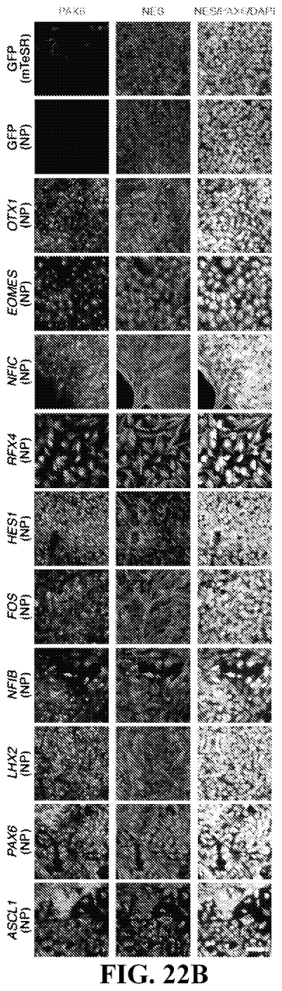

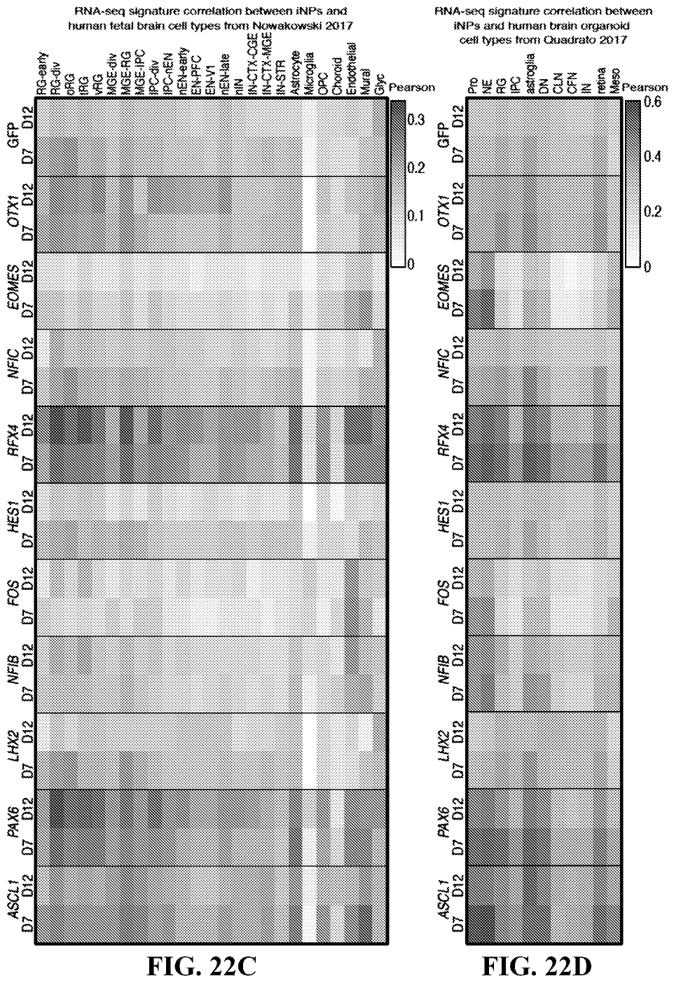

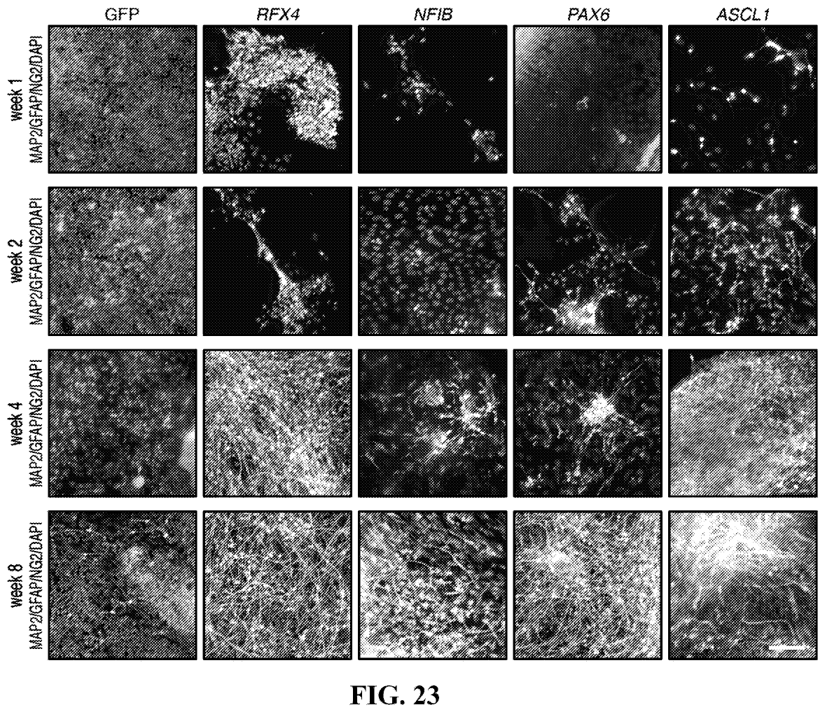

To validate the screening results, Applicants chose to focus on the eight candidate TFs from the flow-FISH screen as well as two additional candidates that were enriched in the other screens and previously suggested to mediate iNP differentiation, ASCL127 and PAX628 (FIG. 13d). Applicants individually overexpressed the top isoform of each TF in hESCs and verified TF expression (FIG. 22a). Immunostaining the iNPs for NP markers showed that all iNPs expressed higher levels of VIM, a gene used to select target cells in the pooled screen, compared to hESCs and exhibited diverse morphologies (FIG. 14a and FIG. 22b). Five candidate TFs (OTX1, EOMES, RFX4, PAX6, and ASCL1) produced iNPs that were morphologically distinct from hESCs overexpressing GFP control, two candidate TFs (HES1 and LHX2) produced iNPs with similar morphologies to hESCs, and three candidate TFs (NFIC, FOS, and NFIB) produced iNPs with morphologies that were in between the two groups. Applicants then compared bulk RNA-seq signatures of iNPs to different cell types in the human fetal cortex or brain organoids^{20,25,26}. Applicants found that transcriptome signatures of iNPs derived using RFX4, ASCL1, and PAX6 were the most similar to NPs, whereas those produced by EOMES and FOS were the most different (FIG. 14b and FIG. 22d, e). The validation results suggest that although overexpression of all candidate TFs upregulated NP marker genes, not all candidate TFs generated cells with transcriptome signatures that resembled those of NPs.

### Example 7—Spontaneous Differentiation of iNPs

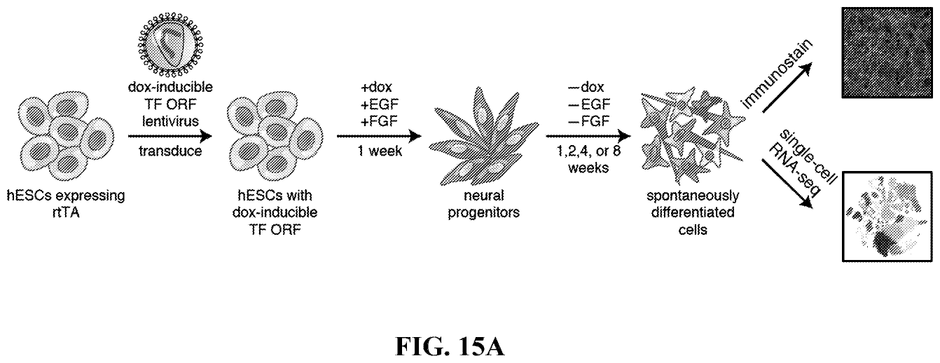

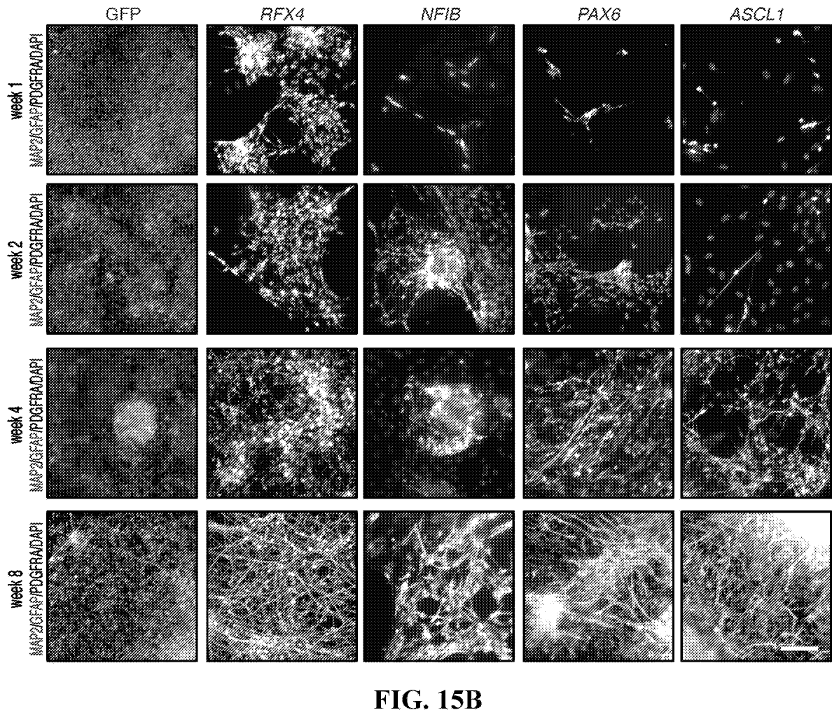

Next, Applicants functionally validated the candidate TFs by spontaneously differentiating the iNPs produced by each candidate. Applicants transiently overexpressed candidate TFs for 1 week to produce iNPs and removed growth factors from the media to allow the iNPs to spontaneously differentiate (FIG. 15a). Functional iNPs, like NPs, should spontaneously differentiate into cell types in the central nervous system (CNS) such as neurons and astrocytes. Out of the ten candidate TFs, four (RFX4, NFIB, PAX6, and ASCL1) produced iNPs that spontaneously differentiated into neurons, astrocytes, and, more rarely, oligodendrocyte precursor cells (FIG. 15b and FIG. 23). Spontaneous differentiation of iNPs generated by these four TFs followed the natural developmental progression of neurogenesis starting at week 1 followed by gliogenesis at week 4 (FIG. 15b and FIG. 23). RFX4 iNPs patterned into neural rosettes prior to neurogenesis (FIG. 15b and FIG. 23).

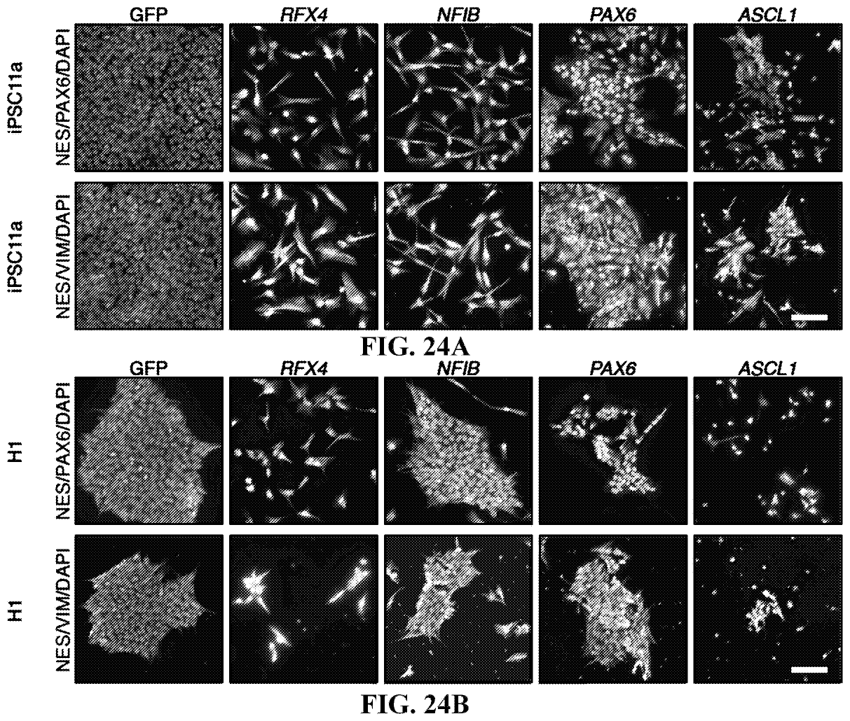

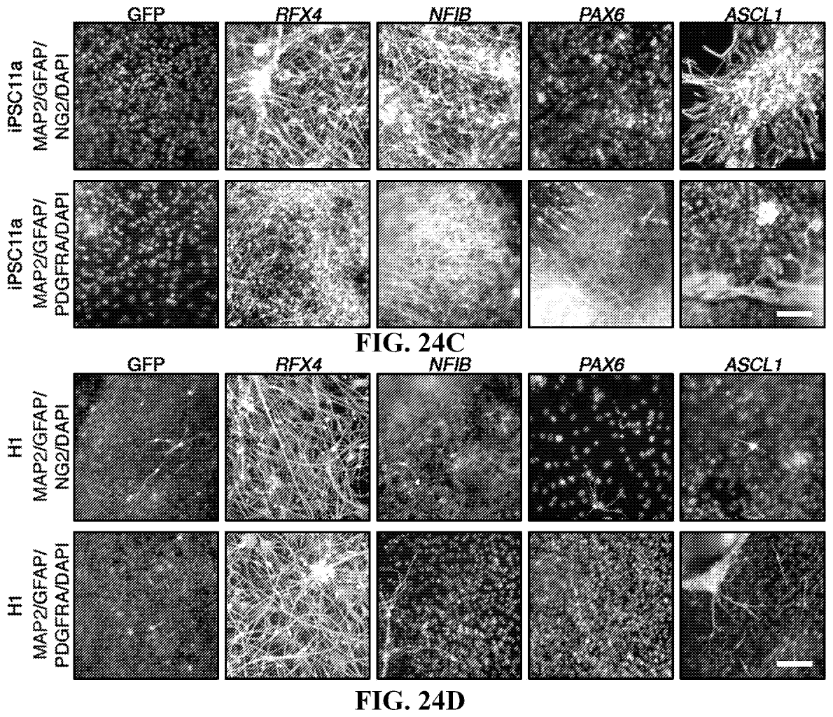

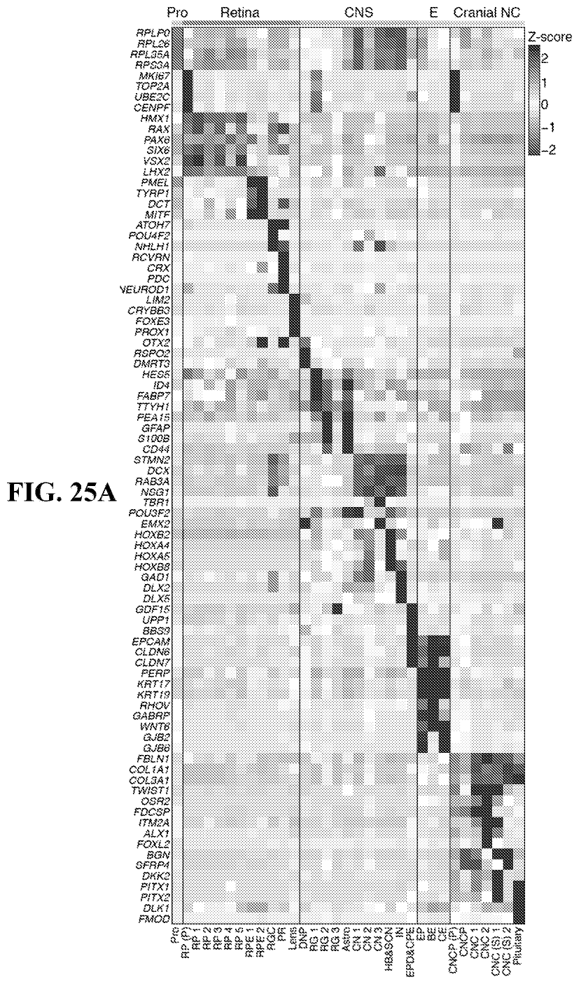

Applicants validated these four TFs in two additional pluripotent stem cell lines, iPSC11a and H1. For both cell lines, overexpression of the four TFs produced iNPs that expressed higher levels of NP marker genes relative to GFP control (FIG. 24a, b). Using spontaneous differentiation to functionally characterize iNPs, Applicants found that RFX4 and NFIB consistently produced functional iNPs in iPSC11a (FIG. 24c), and RFX4 produced functional iNPs in H1 (FIG. 24d). These results indicate that the effects of some TFs are cell line-dependent, while others, like RFX4, are cell line-independent and more likely to play critical roles in NP specification during development.

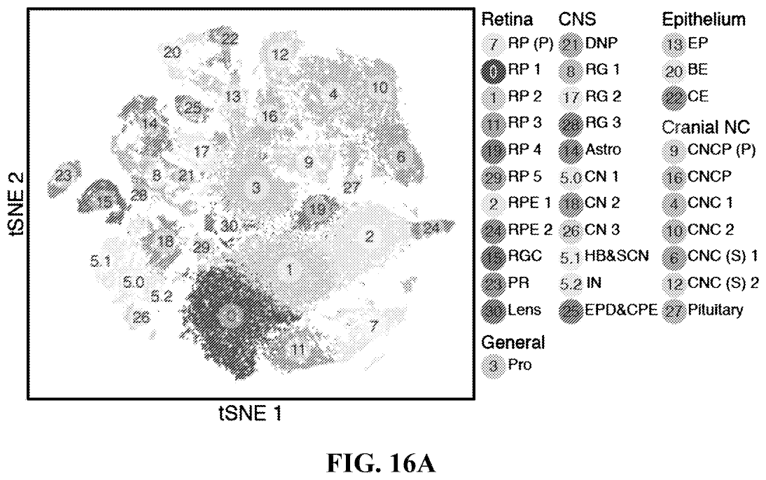

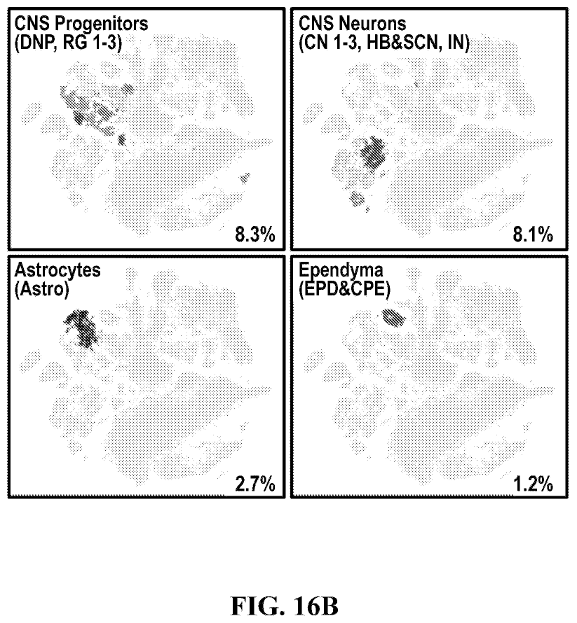

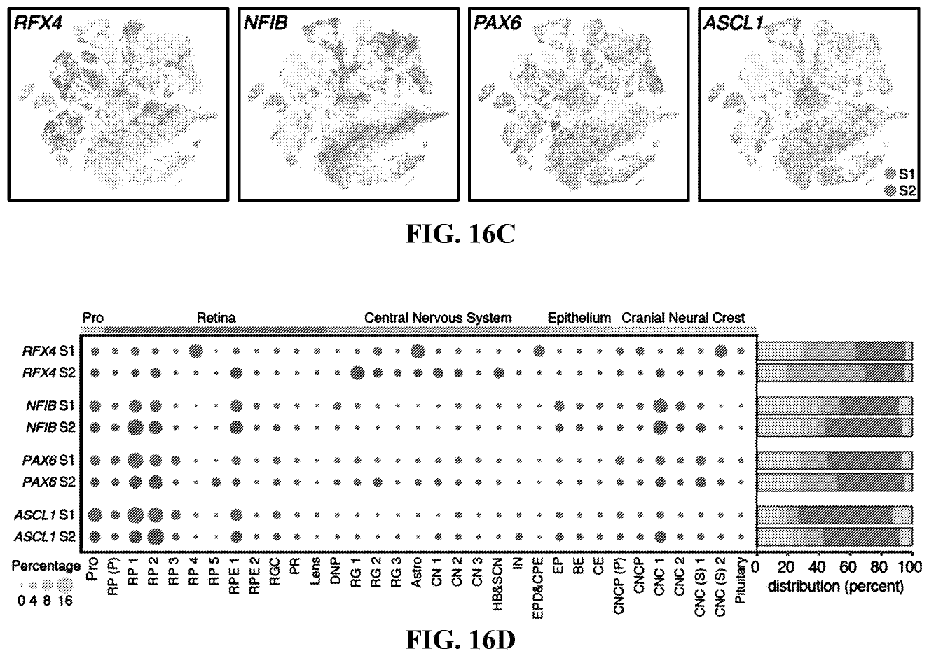

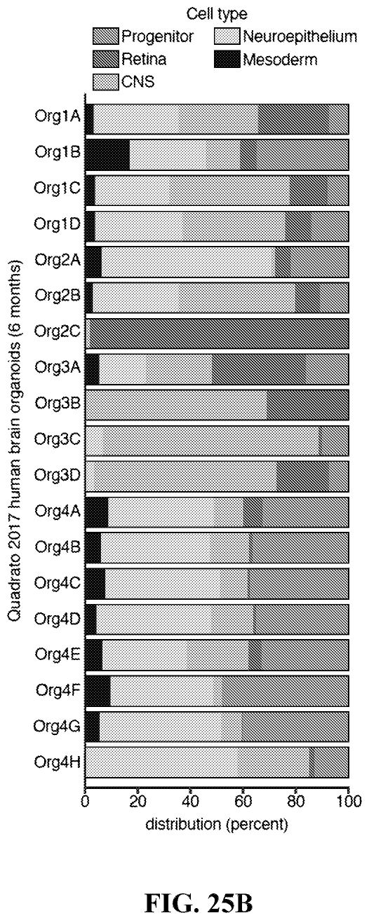

Applicants further characterized the cells spontaneously differentiated from iNPs produced by these four TFs using scRNA-seq. Cluster analysis of 52,364 cells revealed that the iNPs generated a broad range of cell types that are produced by NPs during development, such as cell types from the retina, CNS, epithelium, and neural crest (FIG. 16a, b, FIG. 25a, and Tables 5 and 6). Applicants found that the spontaneously differentiated cell types were generally consistent between biological replicates and distinct between TFs (FIG. 16c, d). RFX4 produced more CNS cell types; NFIB produced more epithelium and neural crest cell types; PAX6 generated cell types in all regions; and ASCL1 produced more retina cell types (FIG. 16c, d). The distributions of cell types generated by TF-iNPs are similar to those of human brain organoids generated with NP embryoid bodies (FIG. 16d and FIG. 25b). Together, the spontaneous differentiation results show that four of the candidate TFs produce functional iNPs.

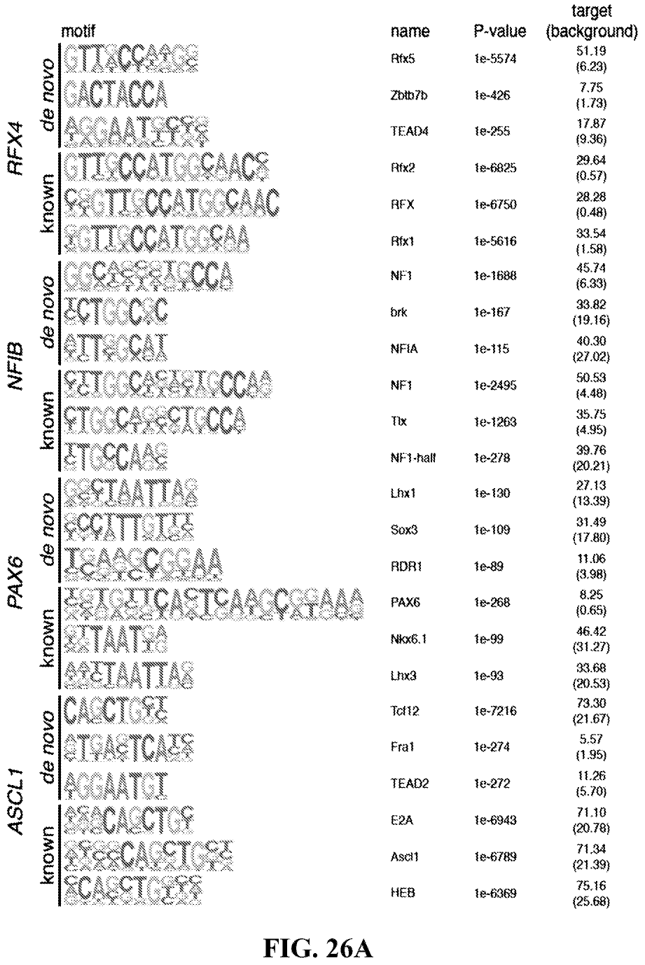

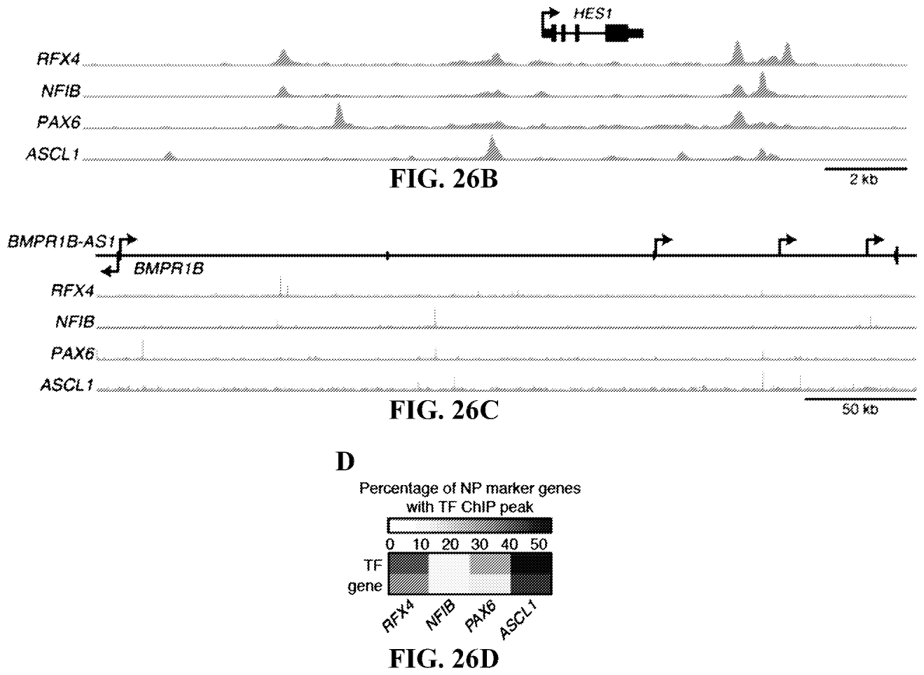

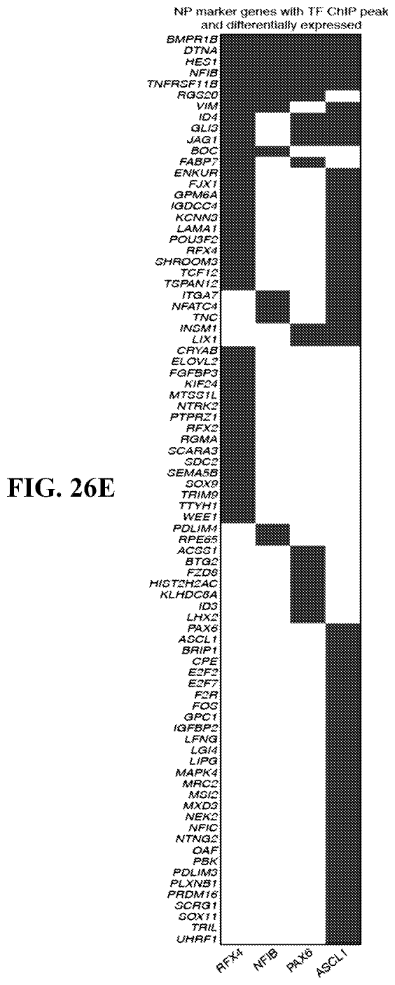

Applicants sought to better understand the transcriptional networks that lead to iNP production by profiling the transcriptional targets of the four TFs using chromatin immunoprecipitation with sequencing (ChIP-seq). Motif analysis generated distinct motifs for each TF and suggested potential transcription coregulators, some of which have been previously shown to interact with the TF (FIG. 26a)^{29,30}. Applicants assigned TFs as potential regulators of a NP marker gene if the TF had a ChIP-seq peak within 10 kb of the gene's transcriptional start site (FIG. 26b-d). For each TF, Applicants identified NP marker genes with TF ChIP-seq peaks that were also differentially expressed upon TF overexpression. Comparison of these NP marker genes between TFs suggested candidate genes that could contribute to the potential mechanisms by which each TF produced iNPs (FIG. 26e). In addition, Applicants found that each of the four TFs had ChIP-seq peaks that were proximal to its own promoter, indicating that each TF positively regulates its own expression to sustain the high expression levels required for differentiation (FIG. 26e).

### Example 8—Modeling Neurodevelopmental Disorders Using iNPs

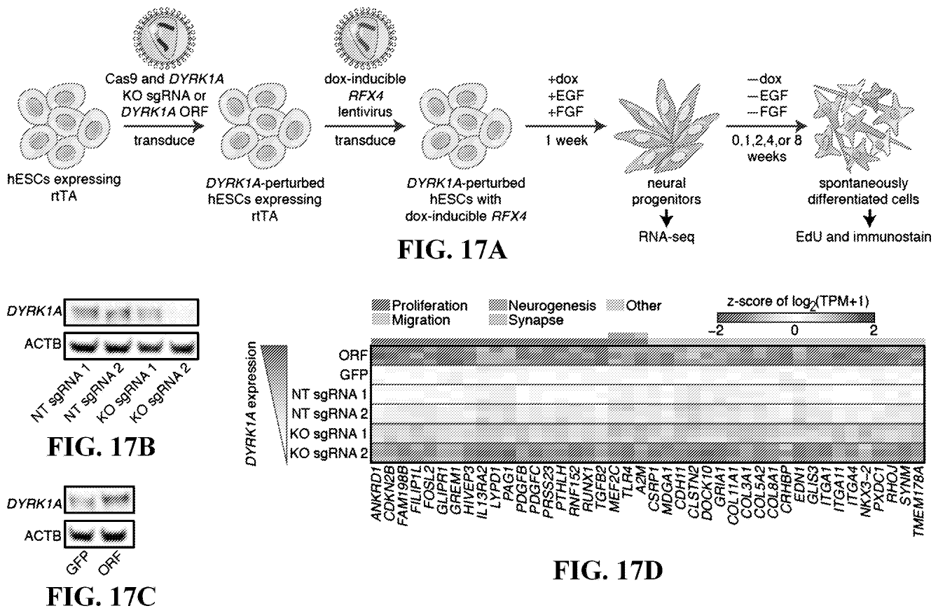

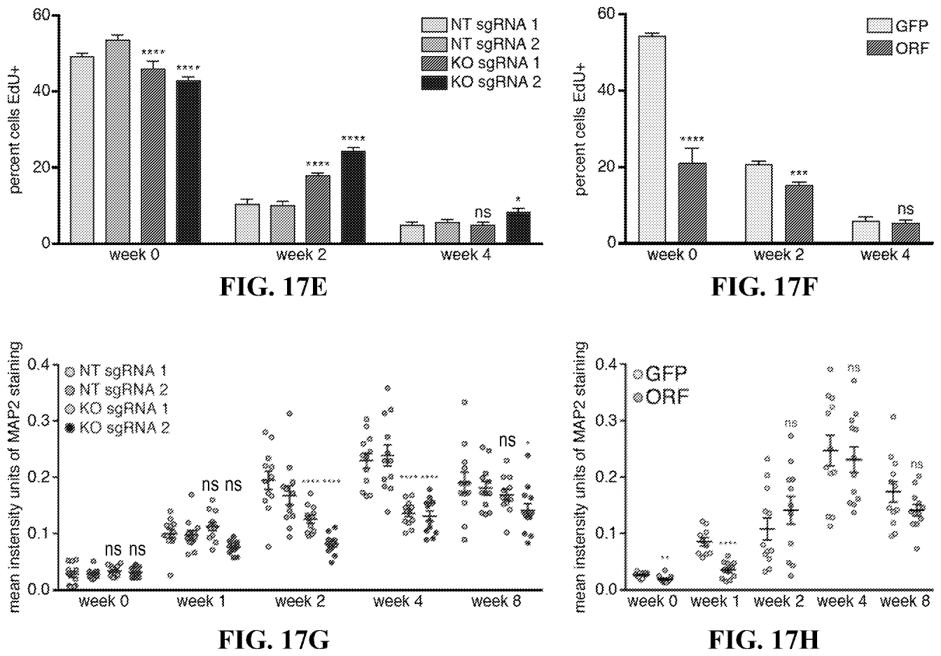

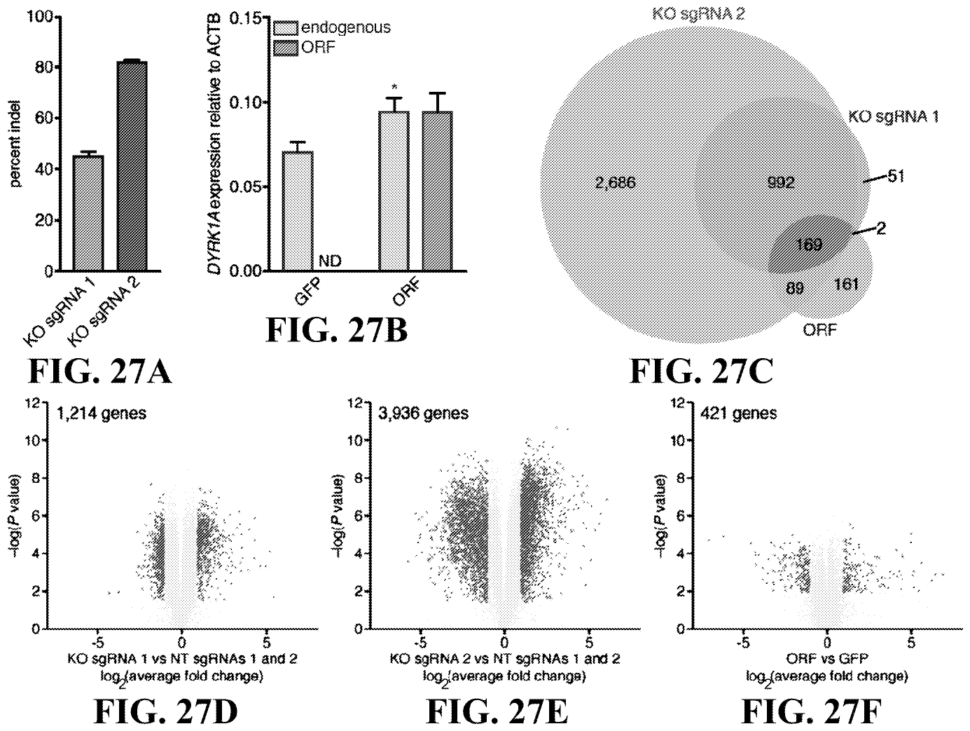

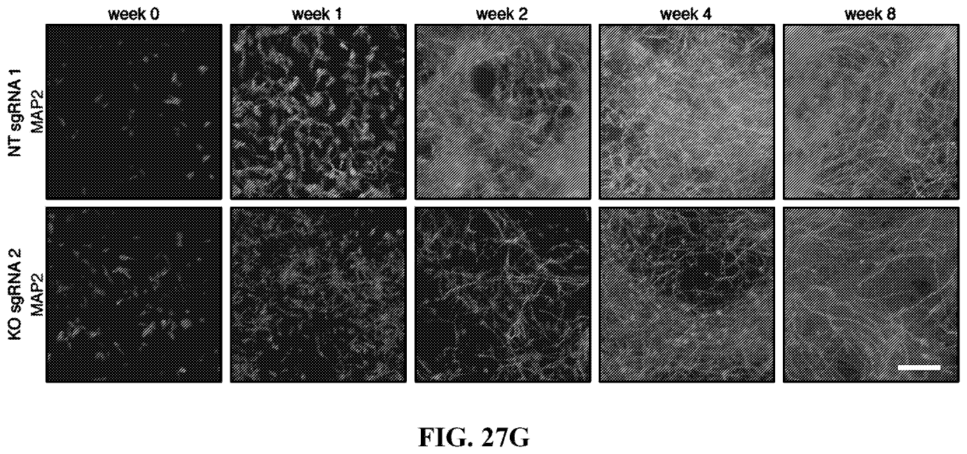

To demonstrate that iNPs can be used to model neurological disorders, Applicants knocked out and overexpressed DYRK1A, perturbations which have been implicated in autism spectrum disorder³¹ and Down syndrome³² respectively, in iPSC11a (FIG. 17a-c and FIG. 27a, b). Applicants transiently overexpressed RFX4 to differentiate the iPSCs into iNPs to study the effects of DYRK1A perturbation on NPs during neural development. Applicants characterized iNPs using bulk RNA-seq and identified genes that were significantly differentially expressed as a result of DYRK1A perturbation (FIG. 17d, FIG. 27c-f, and Table 7). Applicants identified 42 genes that showed DYRK1A dosage-dependent expression changes, some of which are known to be involved in cellular proliferation, neuronal migration, and synapse formation (FIG. 17d).

Applicants then spontaneously differentiated the iNPs to further profile the effects of DYRK1A perturbation on neurogenesis and neural development. Applicants found that knockout of DYRK1A increased, whereas overexpression of DYRK1A decreased, the proportion of proliferating iNPs (FIG. 17e, f), consistent with results from previous studies of DYRK1A perturbation in different model systems³³⁻³⁷. At week 0 of spontaneous differentiation, DYRK1A knockout iNPs showed reduced proliferation, potentially due to toxicity of DNA double-strand breaks introduced by Cas9 (FIG. 17e). However, at weeks 2 and 4, DYRK1A knockout iNPs showed significantly increased proportions of proliferating cells, indicating that more iNPs were actively dividing instead of undergoing neurogenesis (FIG. 17e). As a result, at weeks 2 and 4, Applicants observed a significant reduction in neuronal MAP2 staining (FIG. 17g and FIG. 27g). In contrast, at weeks 0 and 2, DYRK1A overexpression iNPs showed lower proportions of proliferating cells (FIG. 17f). Since there are fewer iNPs due to lower initial proliferation, Applicants observed significant reductions in neuronal MAP2 staining at weeks 0 and 1 (FIG. 17h). Collectively, the DYRK1A perturbation experiments demonstrate that RFX4-iNPs can be used to model effects of perturbations on neural development and neurogenesis, advancing our understanding of complex neurological disorders.

### Example 9—Genome-Scale TF Screen to Identify Drivers of Astrocyte Differentiation

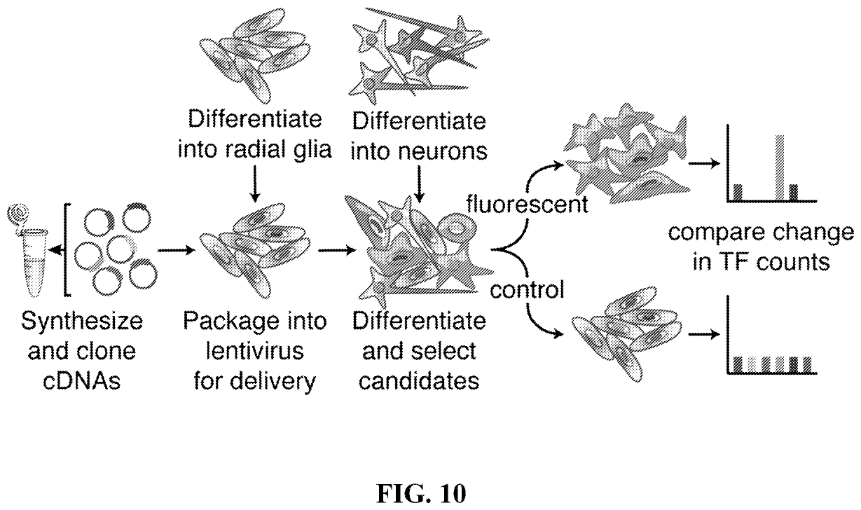

Astrocytes are the most abundant cell type in the vertebrate central nervous system. Although previously thought to be passive responders of neuronal damage, growing evidence suggests that astrocytes actively signal to neurons to influence synaptic development, transmission, and plasticity through secreted and contact-dependent signals (Chung W S, et al., 2015). Current protocols to differentiate astrocytes from hESCs are labor-intensive, requiring the production of embryoid bodies, and take several months to produce mature astrocytes (Krencik R, et al., 2011). Identification of TFs that direct astrocyte differentiation can enable better understanding of astrocyte development and contribute to more complete models of the brain amenable to high-throughput studies. Therefore, Applicants can apply the genome-scale TF screens described herein to identify candidates that can differentiate radial glia into astrocytes (FIG. 10). In addition, performing the astrocyte differentiation screen using the radial glia developed in Examples 1 and 2, 3, 4 can validate the radial glia as a robust model for high-throughput screening.

Using the methods described in Example 2, Applicants have engineered two different HUES66 hESC reporter lines that express the fluorescent protein EGFP upon upregulation of an astrocyte marker gene, either ALDH1L1 or GFAP. For each reporter line, Applicants generated three clonal lines and verified fluorescence upon marker gene upregulation using CRISPR activation. Flow-FISH using astrocyte markers and scRNA-seq may also be used as described.

### Genome-Scale TF Screen for Astrocyte Differentiation

Applicants can differentiate both the GFAP and ALDH1L1 hESC reporter lines or hESCs into radial glia using dox-inducible overexpression of the top radial glia candidate TF(s) found in Examples 1-9. Once the hESC cells have differentiated into radial glia, Applicants can withdraw dox to turn off overexpression and transduce the cells with the genome-scale TF library. Since neurogenesis precedes gliogenesis in the developing brain, Applicants hypothesize that astrocyte differentiation might require signaling from neurons. Applicants can thus perform the TF screen in the presence of neurons differentiated through NEUROG2 overexpression (Zhang Y, et al., 2013). Astrocyte differentiation might also require more time than radial glia differentiation, so Applicants can perform small-scale screens to determine the optimal time point. After 1, 2, and 4 weeks of differentiation, Applicants can use flow cytometry to quantify the percentage of fluorescent cells. Applicants can then perform the genome-scale screen and, at the time point with the highest percentage of fluorescent cells, Applicants can isolate fluorescent cells indicating upregulation of the marker gene and cells with the lowest 15% of fluorescence as controls. Applicants can deep sequence the TF barcodes in both populations to identify TFs enriched in the fluorescent population.

### Validation of Candidate TFs

After identifying candidate TFs for astrocyte differentiation, Applicants can evaluate the fidelity of astrocytes differentiated from these candidates using RNA-seq, immunostaining, and functional studies on synapse formation and elimination. Applicants can perform RNA-seq on the differentiated astrocytes at two different time points determined by enrichment of fluorescent cells during the screen. Applicants can compare the RNA-seq results from differentiated astrocytes to those from human astrocytes using methods described in Example 1-2. Applicants can also immunostain the differentiated astrocytes for astrocyte markers SOX9, AQP4, and GFAP. Finally, Applicants can assess the ability of differentiated astrocytes to promote synapse formation and elimination. For synapse formation, Applicants can culture isolated mouse neurons or differentiated human neurons with and without the differentiated astrocytes and quantify the number of synapses in each condition by immunostaining for pre- and post-synaptic markers bassoon and homer1, respectively, and imaging. Applicants can quantify synapse elimination with an in vitro assay used in previous studies where Applicants conjugate a pH-sensitive fluorescent dye (pHrodo) to isolated synaptosomes that fluoresce upon incorporation into lysosomes through phagocytosis (Chung W S, et al., Astrocytes mediate synapse elimination through MEGF10 and MERTK pathways. Nature. 2013; 504(7480):394-400).

### Discussion

Like radial glia, astrocytes in the human brain are very diverse, and Applicants therefore expect to find multiple TFs that direct differentiation into different subtypes of astrocytes. These TFs can likely regulate cellular pathways that are important for astrocyte function. Like in vivo astrocytes, the differentiated astrocytes can potentially increase synapse formation and phagocytose synaptosomes.

Since astrocytes arise at a later time point than radial glia during development, Applicants may extend the differentiation time of the pooled screen accordingly. In addition, it is possible that astrocyte differentiation requires exogenous factors beyond those provided by NEUROG2-differentiated neurons. Applicants can screen in the presence of isolated mouse neurons or mouse cortical brain slices to provide additional factors. If astrocyte differentiation requires upregulation of more than one TF, Applicants can transduce the TF library at high MOI. Applicants can also combine TF upregulation with downregulation by generating a TF CRISPR knockdown library and transducing cells with both the cDNA and CRISPR knockdown libraries.

### Example 10—Discussion

In summary, Applicants have developed a systematic method to identify TFs for iNP differentiation that could be applied to any cell type of interest. Applicants showed that Applicants could start with NP RNA-seq data to select TFs and marker genes for unbiased pooled screening. Applicants demonstrated feasibility of using reporter cell line, flow-FISH, or scRNA-seq methods to select candidate TFs. Applicants found four novel TFs that could individually differentiate hESCs and iPSCs into iNPs that resemble the morphology, transcriptome signature, and functionality of human fetal radial glia. Out of the four candidate TFs, RFX4-derived iNPs spontaneously differentiated into the highest proportion of CNS cell types, although relative to the other candidates RFX4 has not been extensively studied in CNS development^{38,39}. The findings thus highlight the importance of performing unbiased TF screens. By knocking out and overexpressing DYRK1A in iNPs to model neurodevelopmental disorders, Applicants demonstrated the potential of iNPs to advance our understanding of complex processes in development and disease.

The screening approach could be extended to generate other cell types that may require more than one TF. To identify combinations of TFs, Applicants could screen TFs at a higher MOI to increase the probability of introducing more than one TF in the same cell. Iterative TF screens, for instance performing TF screens in iNPs for differentiation into neurons or glia, may more closely mimic the natural developmental trajectory and facilitate generation of mature cell types. Other factors, such as mechanical stress or signaling from other cell types that are naturally present during development, may also be necessary in TF screens for some cell types.

Beyond cellular programming, TF screening enables identification of factors involved in cellular reprogramming and trans-differentiation, as well as cancer progression and senescence. The demonstration that barcoding of ORFs allows for a variety of screening selection methods could also apply to pooled ORF screening of other protein families of interest. Future application of this TF screening platform for cellular engineering has the potential to expand the number of available cellular models that will help elucidate complex regulatory mechanisms behind development and disease.

### Example 11—TF Screen to Identify Drivers of Cardiomyocyte Differentiation

Using the described screens, Applicants have identified that the transcription factor EOMES generates cardiomyocytes. Overexpression of EOMES for 2 days differentiates stem cells into beating cardiomyocytes by 8 days. This differentiation method produces much higher percentages of cardiomyocytes (˜75% vs ˜30%) than the published mouse method (see, e.g., Van den Ameele J, Tiberi L, Bondue A, et al. Eomesodermin induces Mesp1 expression and cardiac differentiation from embryonic stem cells in the absence of Activin. EMBO Reports. 2012; 13(4):355-362. doi:10.1038/embor.2012.23; and WO2013010965A1). The present invention has demonstrates using human EOMES for differentiating human stem cells. For the cardiomyocytes, Applicants have observed the cells beating after 2 weeks of differentiation and have made a video recording. Applicants have also further identified MESP1 and ESR1 as candidates that drive cardiomyocyte differentiation. In certain embodiments, the cardiomyocytes generated according to the present invention may be used for transplant into patients suffering from heart disease. The present methods also allow for generating cardiomyocytes in a method requiring the expression of a single transcription factor as opposed to previous methods requiring fibroblasts to be differentiated into cardiomyocytes by expressing three transcription factors. In certain embodiments, the cardiomyocytes of the present invention may be used for screening drugs. For example, drugs that are toxic to cardiomyocytes can be screened.

Conditions for generating cardiomyocytes according to the present invention include the following. Culturing ES cells in RPMI+1X B27(without insulin)+50 ug/ml ascorbic acid; switch to RPMI+1×B27 at day 7. The seeding density is high (about 500,000 cells/mL). Dox (about 500 ng/ml) is added to induce expression of the transcription factor (e.g., EOMES) between or at days 0-2. This method results in about 75% of the cells expressing the cardiomyocyte marker TNNT2.

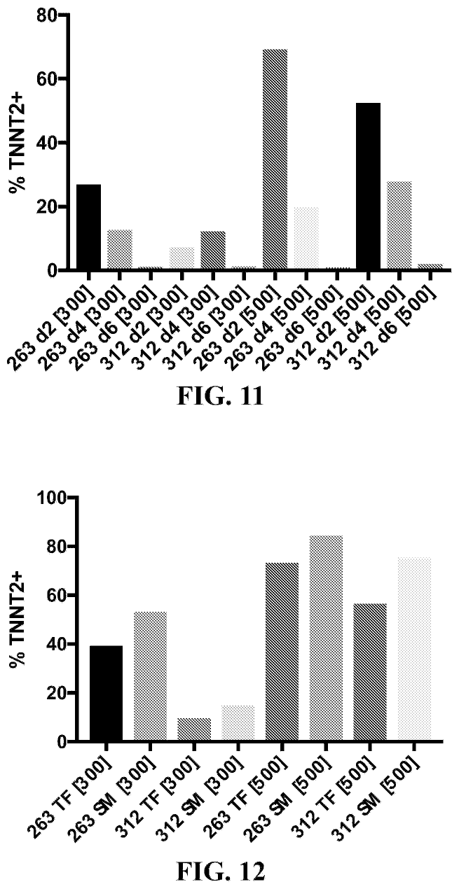

FIG. 11 shows an experiment differentiating cardiomyocytes with different concentrations of Dox to express two different EOMES isoforms. Applicants measured the percentage of cells expressing TNNT2 (Troponin T, cardiomyocyte marker) by fixing cells, staining with TNNT2 antibodies, and quantifying using flow cytometry at 10 days after the start of dox induction. As used herein, 263 refers to EOMES isoform NM_005442 (SEQ ID NO: 10807) and 312 refers to EOMES isoform NM_001278182 (SEQ ID NO: 10808). As used herein, d2, d4, and d6 refers to 2 days, 4 days, and 6 days of dox induction respectively. As used herein, and refer to cell seeding density at 300,000 cells/mL and 500,000 cells/mL. In conclusion, FIG. 11 shows that 2 days of dox induction at 500,000 cells/mL are required for high efficiency differentiation of cardiomyocytes for the 263 and 312 isoforms.

(SEQ ID NO: 10807
MQLGEQLLVSSVNLPGAHFYPLESARGGSGGSAGHLPSAAPSPQK
LDLDKASKKFSGSLSCEAVSGEPAAASAGAPAAMLSDTDAGDAFA
SAAAVAKPGPPDGRKGSPCGEEELPSAAAAAAAAAAAAAATARYS
MDSLSSERYYLQSPGPQGSELAAPCSLFPYQAAAGAPHGPVYPAP
NGARYPYGSMLPPGGFPAAVCPPGRAQFGPGAGAGSGAGGSSGGG
GGPGTYQYSQGAPLYGPYPGAAAAGSCGGLGGLGVPGSGFRAHVY
LCNRPLWLKFHRHQTEMIITKQGRRMFPFLSFNINGLNPTAHYNV
FVEVVLADPNHWRFQGGKWVTCGKADNNMQGNKMYVHPESPNTGS
HWMRQEISFGKLKLTNNKGANNNNTQMIVLQSLHKYQPRLHIVEV
TEDGVEDLNEPSKTQTFTFSETQFIAVTAYQNTDITQLKIDHNPF
AKGFRDNYDSSHQIVPGGRYGVQSFFPEPFVNTLPQARYYNGERT
VPQTNGLLSPQQSEEVANPPQRWLVTPVQQPGTNKLDISSYESEY
TSSTLLPYGIKSLPLQTSHALGYYPDPTFPAMAGWGGRGSYORKM
AAGLPWTSRTSPTVFSEDQLSKEKVKEEIGSSWIETPPSIKSLDS
NDSGVYTSACKRRRLSPSNSSNENSPSIKCEDINAEEYSKDTSKG
MGGYYAFYTTP
(SEQ ID NO: 10808)
MQLGEQLLVSSVNLPGAHFYPLESARGGSGGSAGHLPSAAPSPQK
LDLDKASKKFSGSLSCEAVSGEPAAASAGAPAAMLSDTDAGDAFA
SAAAVAKPGPPDGRKGSPCGEEELPSAAAAAAAAAAAAAATARYS
MDSLSSERYYLQSPGPQGSELAAPCSLFPYQAAAGAPHGPVYPAP
NGARYPYGSMLPPGGFPAAVCPPGRAQFGPGAGAGSGAGGSSGGG
GGPGTYQYSQGAPLYGPYPGAAAAGSCGGLGGLGVPGSGFRAHVY
LCNRPLWLKFHRHQTEMIITKQGRRMFPFLSFNINGLNPTAHYNV
FVEVVLADPNHWRFQGGKWVTCGKADNNMQGNKMYVHPESPNTGS
HWMRQEISFGKLKLTNNKGANNNNTQMIVLQSLHKYQPRLHIVEV
TEDGVEDLNEPSKTQTFTFSETQFIAVTAYQNTDITQLKIDHNPF
AKGFRDNYDSMYTASENDRLTPSPTDSPRSHQIVPGGRYGVQSFF
PEPFVNTLPQARYYNGERTVPQTNGLLSPQQSEEVANPPQRWLVT
PVQQPGTNKLDISSYESEYTSSTLLPYGIKSLPLQTSHALGYYPD
PTFPAMAGWGGRGSYQRKMAAGLPWTSRTSPTVFSEDQLSKEKVK
EEIGSSWIETPPSIKSLDSNDSGVYTSACKRRRLSPSNSSNENSP
SIKCEDINAEEYSKDTSKGMGGYYAFYTTP

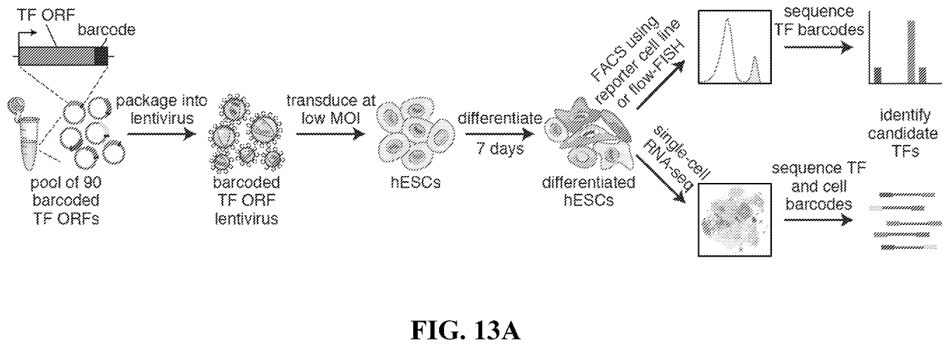

FIG. 12 shows an experiment comparing the differentiating cardiomyocytes by the methods according to the present invention and differentiation by using a small molecule method. Applicants measured the percentage of cells expressing TNNT2 by fixing cells, antibody staining, and quantifying using flow cytometry at 10 days after the start of dox induction. TF refers to adding dox and over expressing the transcription factor EOMES for 2 days. SM refers to an optimized version of a published small molecule differentiation method (Lian et al., Directed cardiomyocyte differentiation from human pluripotent stem cells by modulating Wnt/β-catenin signaling under fully defined conditions, Nature Protocols volume 8, pages 162-175 (2013) doi:10.1038/nprot.2012.150). Applicants determined that the method according to the present invention using the 263 TF conditions is comparable to 263 SM method. Further studies also show differentiation of human pluripotent stem cells (hPSCs) to cardiomyocytes using small molecules (see, e.g., Karakikes, et al., Small molecule-mediated directed differentiation of human embryonic stem cells toward ventricular cardiomyocytes, Stem Cells Transl Med. (2014); Sharma, et al., Derivation of highly purified cardiomyocytes from human induced pluripotent stem cells using small molecule-modulated differentiation and subsequent glucose starvation, J Vis Exp. (2015); and Burridge, et al., Chemically Defined Culture and Cardiomyocyte Differentiation of Human Pluripotent Stem Cells. Curr Protoc Hum Genet. (2015)).

### Example 12—A Multiplexed Transcription Factor Screening Platform for Directed Differentiation

Directed differentiation of human pluripotent stem cells into diverse cell types has the potential to realize a broad array of cellular replacement therapies and provides a tractable model that can be perturbed, genetically or chemically, to assess effects in a cell type-specific context (Cohen and Melton, 2011; Colman and Dreesen, 2009; Keller, 2005; Kiskinis and Eggan, 2010; Robinton and Daley, 2012). However, it remains challenging or impossible to generate many cell types (Cohen and Melton, 2011; Colman and Dreesen, 2009; Keller, 2005; Kiskinis and Eggan, 2010; Robinton and Daley, 2012). The best differentiation methods are often labor-intensive and can require months to produce even heterogenous or immature cell populations. Many of these methods rely on exogenous growth factors or small molecules, which are often dosage-sensitive and difficult to identify in a scalable manner. Alternatively, overexpression of transcription factors (TFs) has been shown to rapidly and efficiently generate many different cell types, including neurons and skeletal muscle cells (Furuyama et al., 2019; Pang et al., 2011; Song et al., 2012; Sugimura et al., 2017; Takahashi and Yamanaka, 2006; Weintraub et al., 1989; Zhang et al., 2013). As TFs use endogenous regulatory pathways to drive differentiation, mimicking natural development, this approach to engineering cell fate may produce higher fidelity models while illuminating aspects of development. However, the process of discovering TFs for directed differentiation relies on time-intensive and low-throughput arrayed screens. Arrayed screens, in which each perturbation must be performed and tested individually, are challenging to carry out at large scale, typically limited to 5-25 TFs (Furuyama et al., 2019; Pang et al., 2011; Song et al., 2012; Sugimura et al., 2017; Takahashi and Yamanaka, 2006; Weintraub et al., 1989; Zhang et al., 2013). By contrast, pooled screening approaches, which make use of barcodes to enable multiple perturbations to be tested in parallel, are more scalable, both in terms of time and cost.

To unlock the potential of this promising approach, Applicants sought to develop a multiplexed TF screening platform to identify TFs that can drive specific cell fates in a high-throughput manner. Applicants explored two requirements for pooled screening to identify TFs that drive differentiation. First, perturbations can be introduced into cells via a single copy to drive sufficient TF expression to induce cellular programing. Second, target cell types can be enriched from a diverse cell population, and the TF perturbations that produce the target cell types can be identified.

Applicants first compared different TF overexpression methods and found that ORF overexpression most effectively differentiated human embryonic stem cells (hESCs) into neurons. To establish a generalizable platform for systematic identification of TFs for cellular programming, Applicants created a barcoded human TF library, which Applicants named Multiplexed Overexpression of Regulatory Factors (MORF). The MORF library consists of all known TFs from the human genome, with 3,548 isoforms covering 1,836 genes, and used this library to assay 90 TF isoforms for differentiation of hESCs into neural progenitors (NPs). Applicants chose NPs as the target cell type because induced NPs (iNPs) offer a tractable model for studying complex disorders of the central nervous system (CNS), but current methods for producing iNPs, namely embryoid body formation (Schafer et al., 2019; Zhang et al., 2001) or dual SMAD inhibition (Chambers et al., 2009; Shi et al., 2012a), are low-throughput or produce variable differentiation results depending on the cell line (Hu et al., 2010), respectively. Applicants selected for TFs that drive iNP differentiation using various methods to enrich for target cell types based on marker gene combinations. The pooled screens identified four TFs (RFX4, NFIB, PAX6, and ASCL1), each of which produced multipotent iNPs that could spontaneously differentiate into CNS cell types. Addition of dual SMAD inhibitors to RFX4-overexpressing cells produced homogenous iNPs that preferentially differentiated into GABAergic neurons. RFX4-iNPs can be used to model neurodevelopmental disorders. Using iNPs as a demonstration, Applicants show that pooled TF screening is a scalable and generalizable approach for systematically identifying TFs that drive differentiation of desired cell types.

### Example 13—TF ORF Overexpression Effectively Drives Differentiation

Recently, the microbial CRISPR-Cas9 system has been adapted for large-scale gene activation screening, which provides a rapid and efficient method for elucidating complex biology at the genome scale (Gilbert et al., 2014; Konermann et al., 2015). Applicants therefore first sought to leverage the ease and scalability of CRISPR activation (CRISPRa) to screen 1,965 annotated TF genes (Zhang et al., 2012) for their ability to drive differentiation of HUES66 hESCs toward NP cell fates. However, the initial screen did not lead to significant differentiation (data not shown), in contrast to previous observations in mouse embryonic stem cells (Liu et al., 2018).

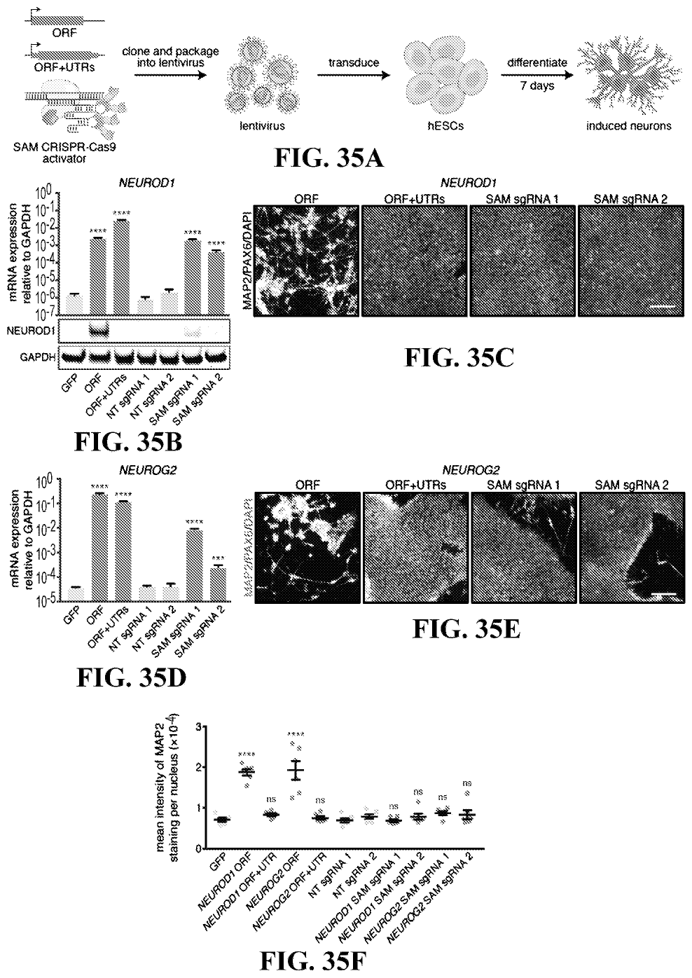

Although CRISPRa has been used in a range of biological contexts (Gilbert et al., 2014; Joung et al., 2017a; Konermann et al., 2015), the particular regulatory environment of hESCs may be uniquely buffered against TF overexpression. Therefore, Applicants next compared the ability of CRISPRa and ORF-based methods to overexpress NEUROD1 or NEUROG2, two TFs that have been previously shown to induce neuronal differentiation (Zhang et al., 2013), at single copy in HUES66 hESCs (FIG. 35A). In order to pinpoint whether expression level or endogenous UTRs were responsible for limiting TF expression, Applicants included ORFs with endogenous UTRs in the comparison with CRISPRa. For both NEUROD1 and NEUROG2, Applicants found that expression of the TF ORF effectively induced neuronal differentiation (FIGS. 35B-F). Surprisingly, Applicants found that overexpression of the TFs using the ORF with endogenous UTRs did not efficiently differentiate hESCs into neurons, despite robust transcriptional upregulation. As Applicants had observed for the large-scale screen, CRISPRa upregulation of NEUROD1 and NEUROG2 did not effectively induce differentiation. These results suggest that there may be endogenous post-transcriptional regulatory mechanisms in hESCs that buffer against TF protein expression (FIGS. 35B-F). Applicants therefore proceeded with TF ORF overexpression for screening.

### Example 14—a Barcoded Human TF Library for Directed Differentiation

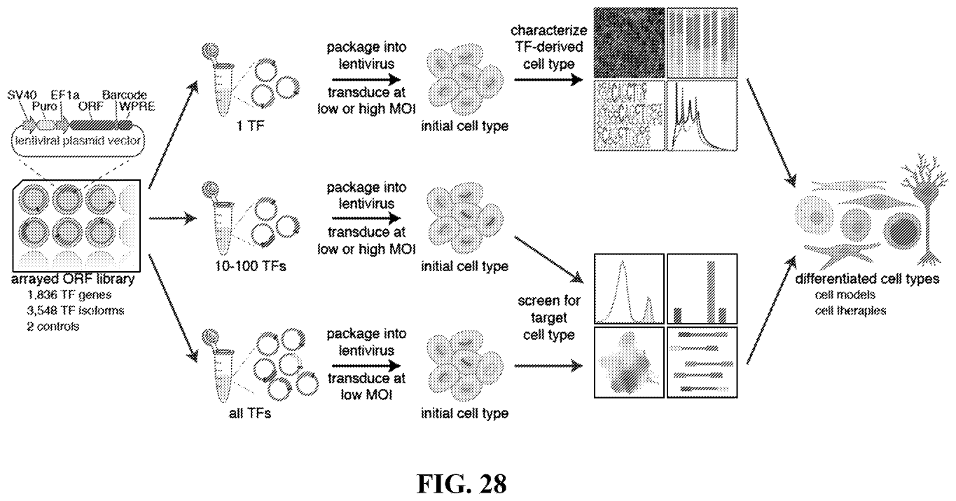

To enable high-throughput, systematic identification of TFs for directed differentiation of any desired cell type, Applicants created a barcoded human TF library, MORF (FIG. 28 and Table 3). The library consists of 1,836 genes, including histone modifiers, and covers 3,548 isoforms that overlap between the RefSeq and GENCODE annotations. Applicants also included two control vectors in the library. All vectors in the library contain unique barcodes that facilitate pooled screening. MORF is provided in an arrayed format that can be readily subpooled for targeted TF screens, followed by characterization of individual candidate TFs. MORF enables a generalizable approach for TF screening that will expand the ability to generate desired cell types.

### Example 15—Development of a Pooled TF ORF Screening Platform for iNP Differentiation

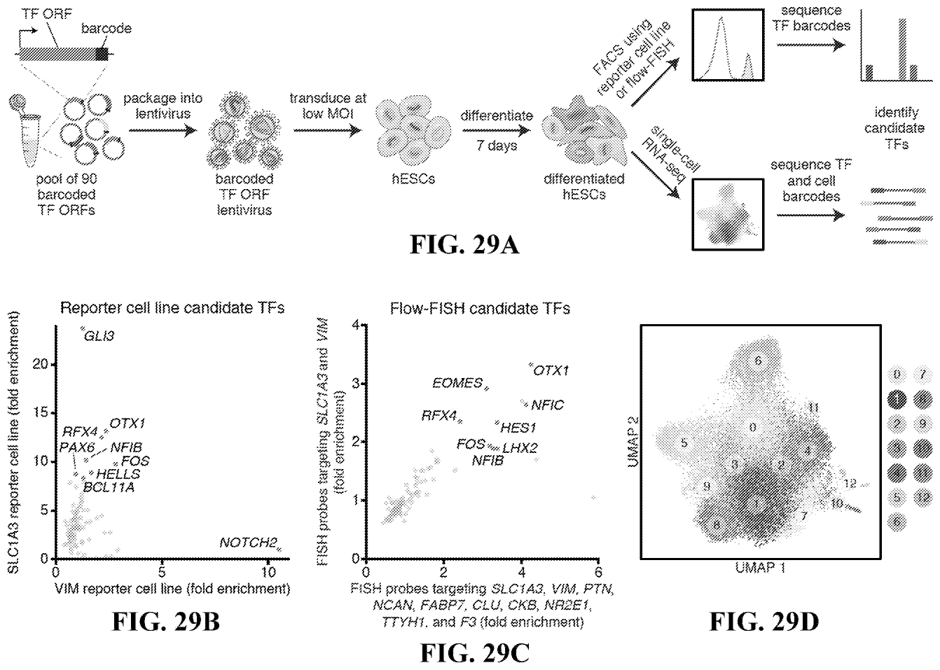

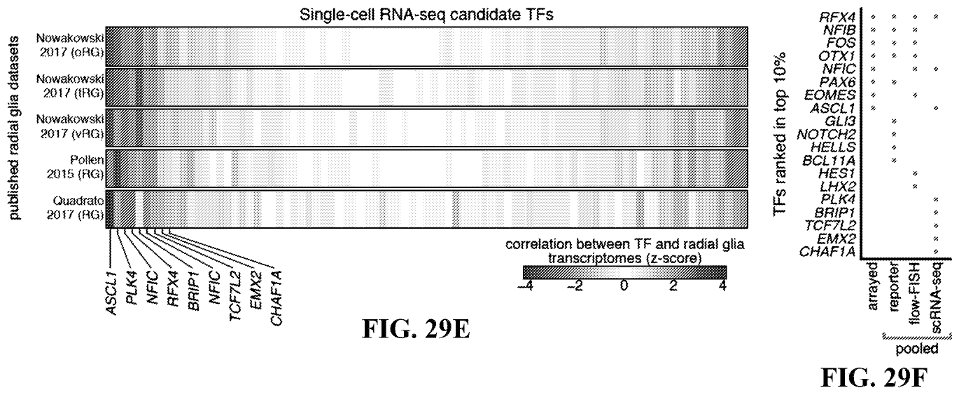

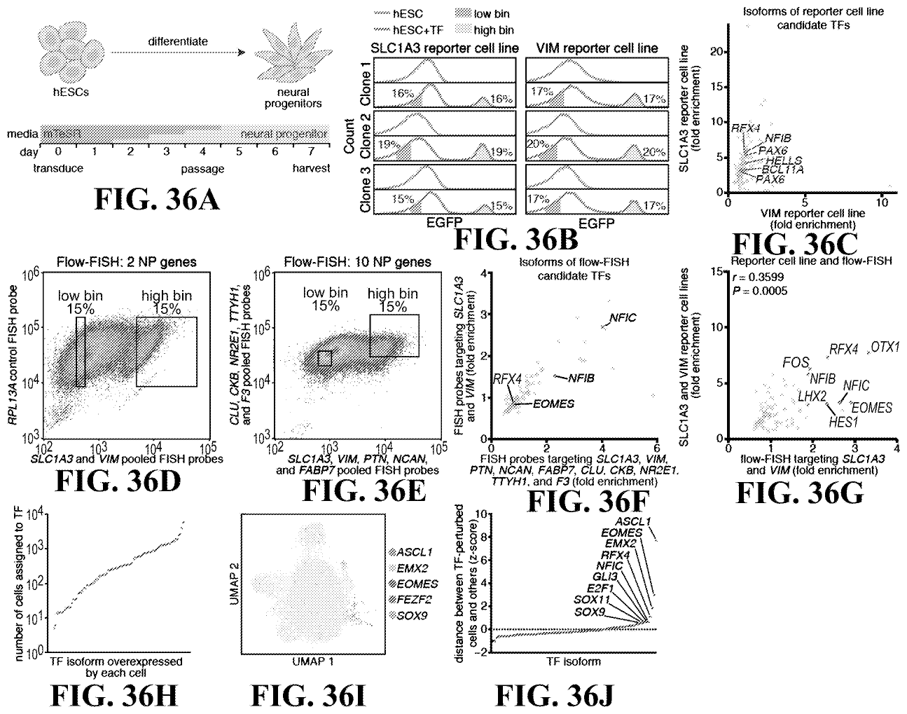

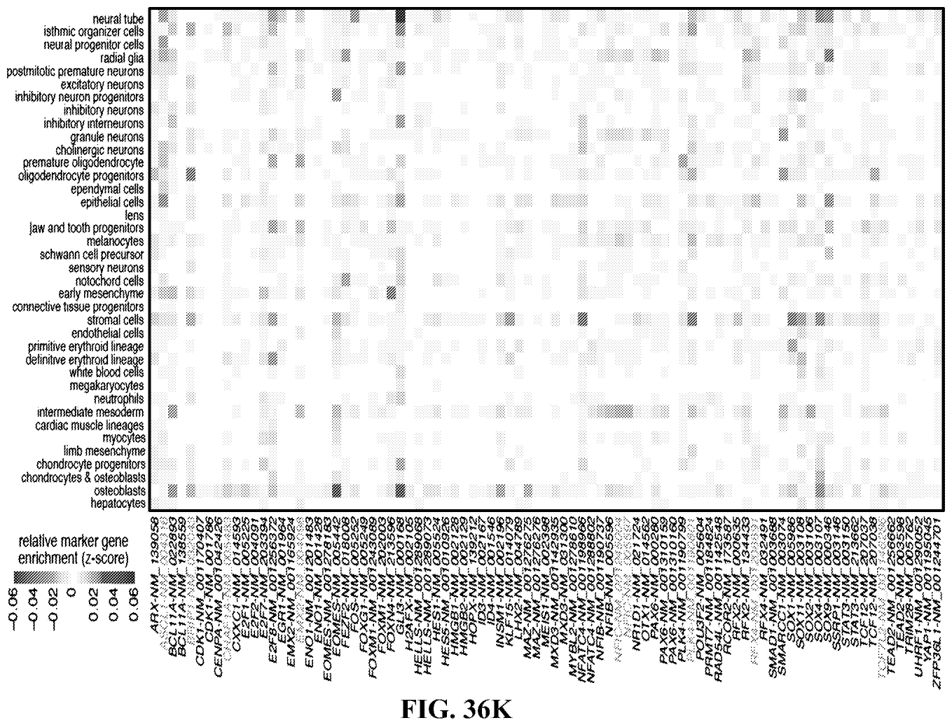

As a demonstration, Applicants performed a targeted TF screen for differentiation of hESCs into iNPs. To select a subset of TFs for the screen, Applicants examined eight RNA-sequencing (RNA-seq) datasets (Camp et al., 2015; Johnson et al., 2015; Llorens-Bobadilla et al., 2015; Pollen et al., 2015; Shin et al., 2015; Thomsen et al., 2016; Wu et al., 2010; Zhang et al., 2016) and identified 70 TFs that were found to be specifically expressed in NPs in at least two datasets. For each TF, Applicants included isoforms that comprised >25% of the expressed transcript in NPs, resulting in a total of 90 TF isoforms (see Methods; Table 1). Applicants pooled the barcoded TFs and packaged them into a lentiviral library for delivery in hESCs (FIG. 29A). Applicants differentiated the cells for 7 days before selecting TFs that drive iNP differentiation (FIG. 36A). To determine the ideal strategy for selecting TFs, Applicants explored three different methods that can simultaneously assay different numbers of marker genes: reporter cell line (1 gene), flow-FISH (2-10 genes), and single-cell RNA-sequencing (scRNA-seq; 10-2,000 genes; FIG. 29A).

For the reporter cell line method, Applicants generated clonal reporter cell lines with EGFP inserted downstream of an endogenous NP marker gene, either SLC1A3 or VIM, which were selected based on convergence across published RNA-seq datasets and high expression levels (Camp et al., 2015; Johnson et al., 2015; Llorens-Bobadilla et al., 2015; Pollen et al., 2015; Shin et al., 2015; Thomsen et al., 2016; Wu et al., 2010; Zhang et al., 2016). Applicants transduced the SLC1A3 or VIM reporter cell line with the pooled TF library, differentiated the cells for 7 days, and sorted for high and low EGFP-expressing cells (FIGS. 29A and 36B). Deep sequencing of the TF barcodes in each population identified candidate TFs that were enriched in the high EGFP-expressing cell population, indicating upregulation of SLC1A3 or VIM (FIGS. 29B and 36C; Table 1).

For the flow-FISH method, Applicants transduced hESCs with the pooled TF library, differentiated the cells for 7 days, and labeled either 2 or 10 NP marker gene transcripts using pooled FISH probes (FIG. 29A). By pooling the FISH probes, Applicants could sort for cells expressing high or low levels of 2-10 marker genes at the same time (FIGS. 36D and 36E). Similar to the reporter cell line method, Applicants deep sequenced the TF barcodes and identified candidate TFs that were enriched in cells expressing higher levels of marker genes (FIGS. 29C and 36F; Table 1). Applicants found that for some TFs, such as EOMES and RFX4, the choice of TF isoform can produce very different differentiation results (FIGS. 29C and 36F). Both the flow-FISH and reporter cell line methods to assay SLC1A3 and VIM expression produced comparable TF enrichment profiles (FIG. 36G).

For the scRNA-seq method, Applicants transduced hESCs with the pooled TF library, differentiated the cells for 7 days, and performed scRNA-seq to profile 53,560 single cells (FIG. 29A). In the barcoded TF ORF vector design, the TF barcode is expressed in the TF mRNA, which is captured by scRNA-seq and can be mapped to cell barcodes (FIG. 28). After assigning TFs to cells, Applicants found that the number of cells that had each TF overexpressed was very skewed, with the top 10% of the TFs having 92 times more cells than the bottom 10% of TFs, potentially due to TF-dependent effects on cell death and proliferation (FIG. 36H). Cluster analysis of the scRNA-seq results suggested that overexpression of several TFs, for instance ASCL1 and EOMES, generated distinct transcriptome signatures that clustered together and were more distant to those of other TFs, while overexpression of most TFs did not produce distinct transcriptome signatures (FIGS. 29D, 361, and 36J). By comparing the TF transcriptome signatures with those published for radial glia (Nowakowski et al., 2017; Pollen et al., 2015; Quadrato et al., 2017), which represent NPs in the developing cortex, Applicants identified candidate TFs with the highest transcriptome signature correlation (FIG. 29E and Table 1). Applicants also compared TF transcriptome signatures to other cell types from the mouse organogenesis cell atlas (Cao et al., 2019) to nominate TFs for additional cell types, such as FOXN4 for early mesenchyme or SOX9 for Schwann cell precursors (FIG. 36K).

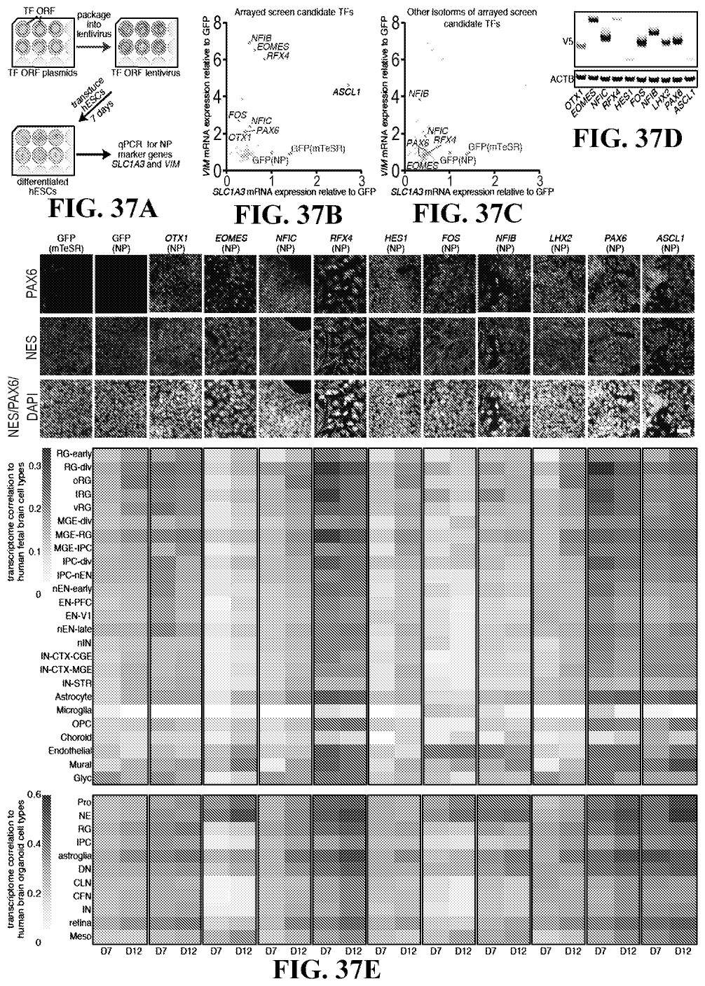

To verify the results from the pooled screen, Applicants performed an arrayed screen on the same 90 TF isoforms, packaging each TF individually into lentivirus for delivery into hESCs (FIG. 37A-C). The arrayed and pooled screens nominated overlapping sets of candidate TFs for iNP differentiation (FIG. 29F and Table 1), some of which (NFIB (Steele-Perkins et al., 2005), OTX1 (Frantz et al., 1994), PAX6 (Englund et al., 2005; Gotz et al., 1998), EOMES (Bulfone et al., 1999; Englund et al., 2005), and ASCL1 (Casarosa et al., 1999)) are known to be involved in neural development, further supporting the screening results. Out of the pooled screening methods, flow-FISH identified the highest number (6 out of 8) of candidate TFs that overlapped with other screens (FIG. 29F). Compared to using reporter cell lines, flow-FISH is more versatile, because the marker gene combinations can be easily exchanged or combined without generating another clonal reporter cell line. Flow-FISH is also more accessible than scRNA-seq and can measure a greater dynamic range of transcript expression. Together, these results suggest that flow-FISH may be an ideal screening method for other cell types.

### Example 16—Validation of Candidate TFs for iNP Differentiation

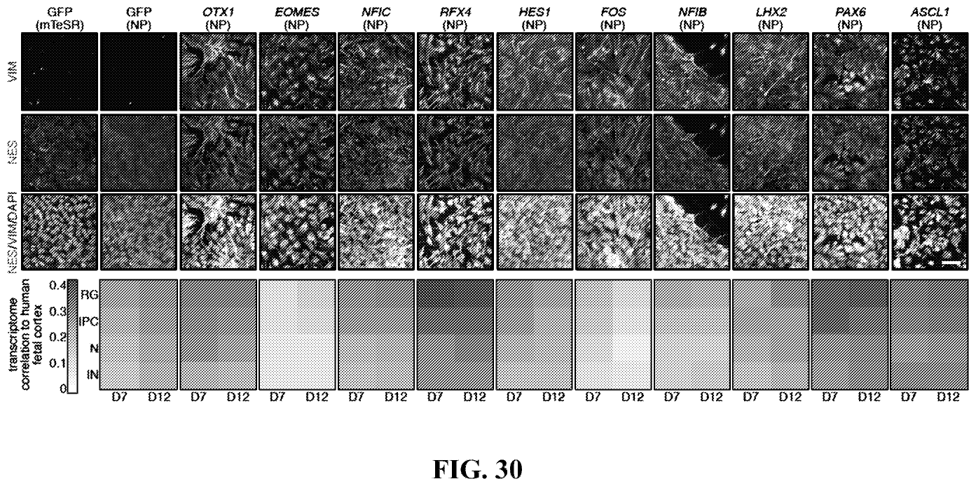

For downstream analysis, Applicants chose to focus on the eight candidate TFs from the flow-FISH screen as well as two additional candidates that were enriched in the other screens and previously suggested to mediate iNP differentiation, ASCL1 (Casarosa et al., 1999) and PAX6 (Zhang et al., 2010) (FIG. 29F). Applicants individually overexpressed the top isoform of each TF in hESCs and verified TF expression (FIG. 37D). Immunostaining the iNPs for NP markers showed that, compared to hESCs, all iNPs expressed higher levels of VIM, a marker used to select target cells in the pooled screen, and exhibited diverse morphologies (FIGS. 30 and 37E). Five candidate TFs (OTX1, EOMES, RFX4, PAX6, and ASCL1) produced iNPs that appear morphologically distinct from hESCs overexpressing GFP control, two candidate TFs (HES1 and LHX2) produced iNPs with similar morphologies to hESCs, and three candidate TFs (NFIC, FOS, and NFIB) produced iNPs with morphologies that were in between the two groups. Applicants then compared bulk RNA-seq signatures of iNPs to different cell types in the human fetal cortex and in brain organoids (Nowakowski et al., 2017; Pollen et al., 2015; Quadrato et al., 2017). Applicants found that transcriptome signatures of iNPs derived using RFX4, ASCL1, and PAX6 were the most similar to NPs, whereas those produced by EOMES and FOS were the most different (FIGS. 30 and 37E; Table 7). Thus, Applicants have validated the pooled screening approach by confirming that overexpression of all candidate TFs upregulated marker genes that are used to enrich for NPs.

### Example 17—Functional Evaluation of iNP Multipotency Using Spontaneous Differentiation

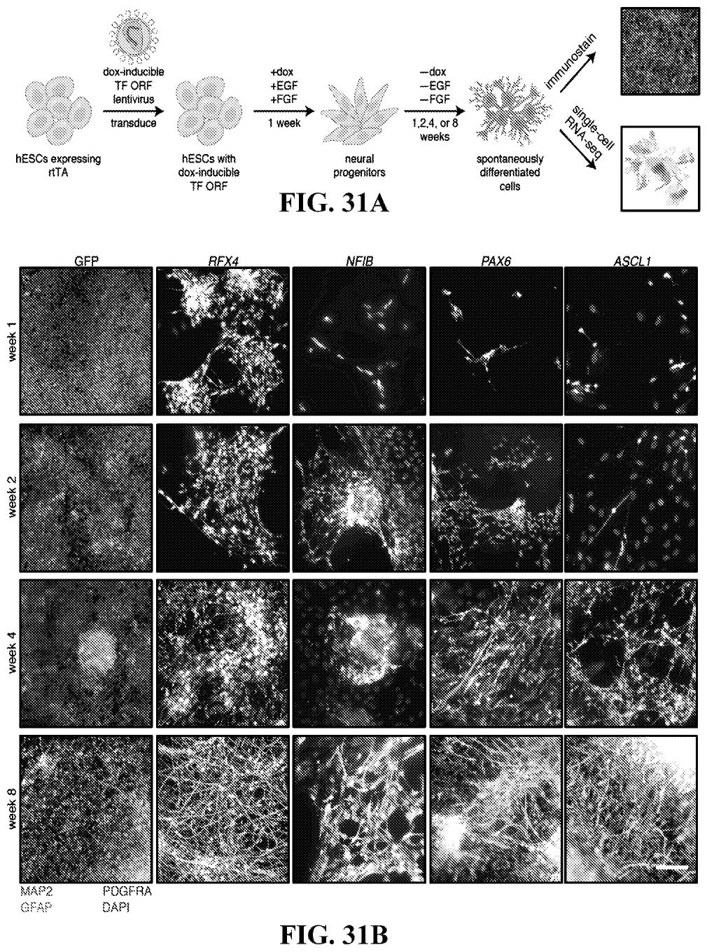

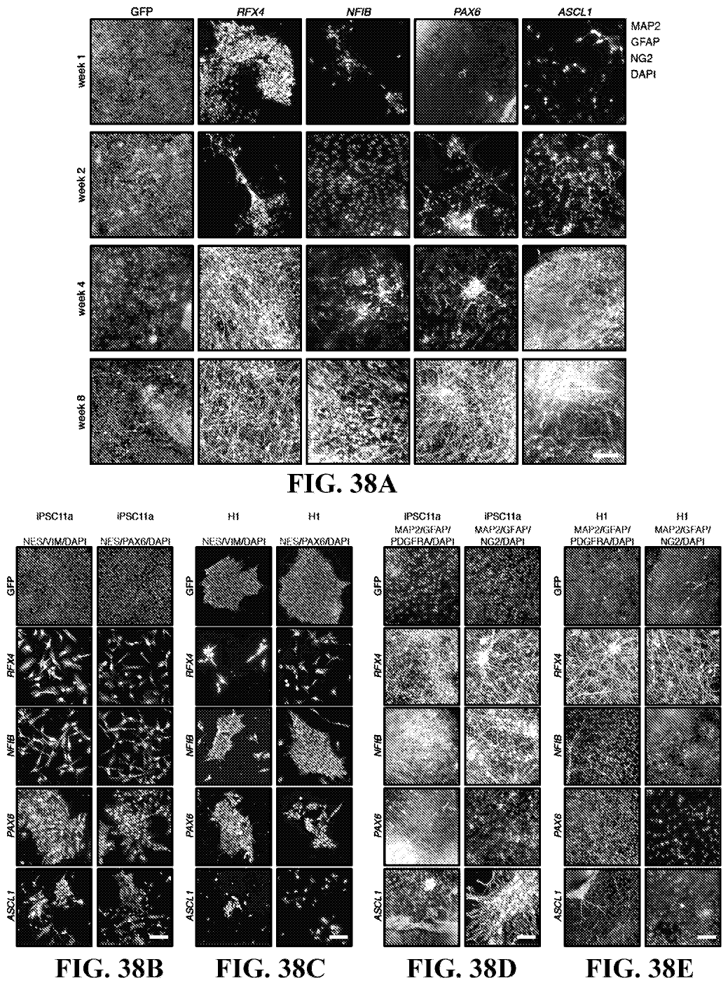

Next, Applicants evaluated the multipotency of iNPs produced by each candidate TF by spontaneously differentiating the iNPs. Applicants transiently overexpressed candidate TFs for 1 week to produce iNPs and then removed growth factors from the media to allow the iNPs to spontaneously differentiate for 8 weeks (FIG. 31A). Like NPs, iNPs should spontaneously differentiate into cell types in the CNS such as neurons and astrocytes. Out of the ten candidate TFs, four (RFX4, NFIB, PAX6, and ASCL1) produced iNPs that spontaneously differentiated into neurons, astrocytes, and, more rarely, oligodendrocyte precursor cells (FIGS. 31B and 38A). Spontaneous differentiation of iNPs generated by these four TFs followed the natural developmental progression of neurogenesis starting at week 1 and proceeding to gliogenesis at week 4 (FIGS. 31B and 38A). RFX4-iNPs patterned into neural rosettes prior to neurogenesis (FIGS. 31B and 38A).

Applicants validated these four TFs in two additional pluripotent stem cell lines, iPSC11a and H1. For both cell lines, overexpression of the four TFs produced iNPs that expressed higher levels of NP marker genes relative to GFP control (FIGS. 38B and 38C). Following spontaneous differentiation, Applicants found that RFX4 and NFIB consistently produced functional iNPs in iPSC11a (FIG. 38D), and RFX4 produced functional iNPs in H1 (FIG. 38E). These results indicate that the effects of some TFs are cell line-dependent, while others, like RFX4, are cell line-independent, which may point to a more critical role in NP specification during development.

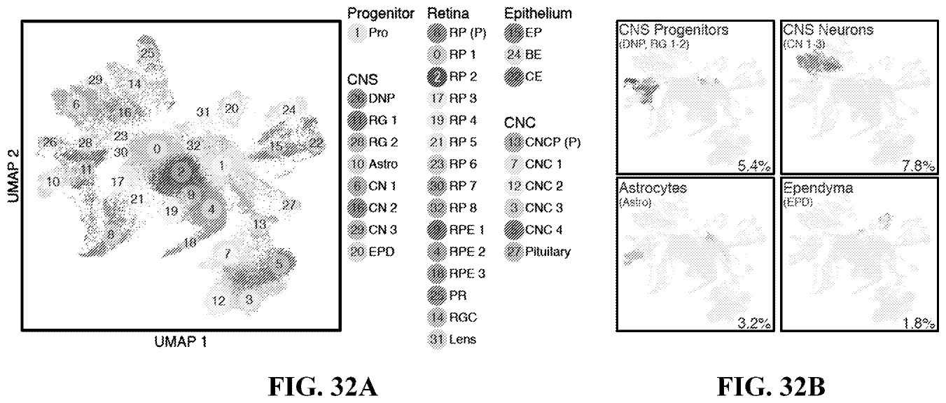

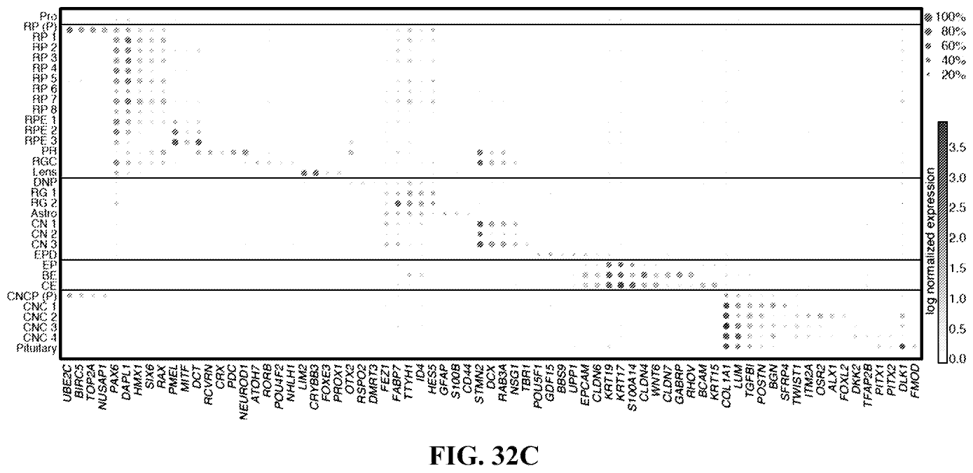

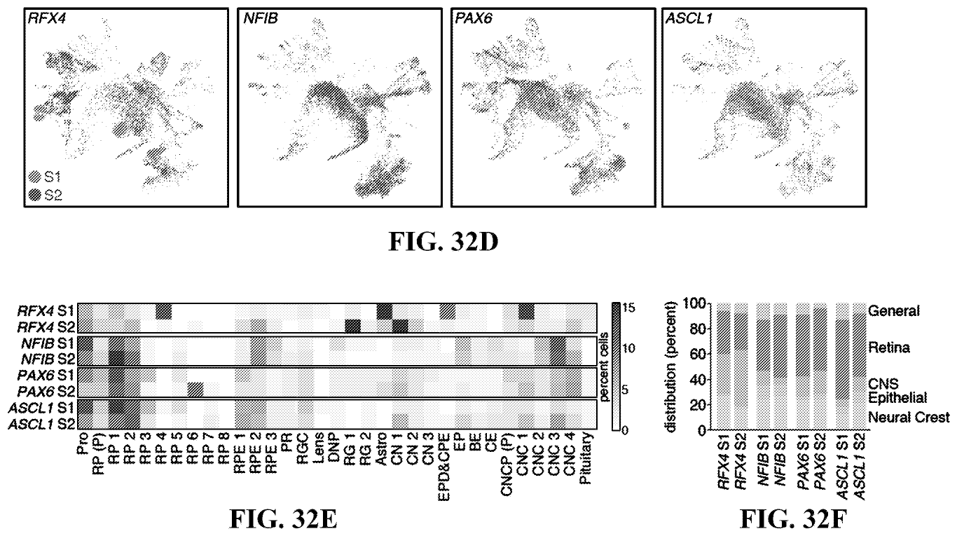

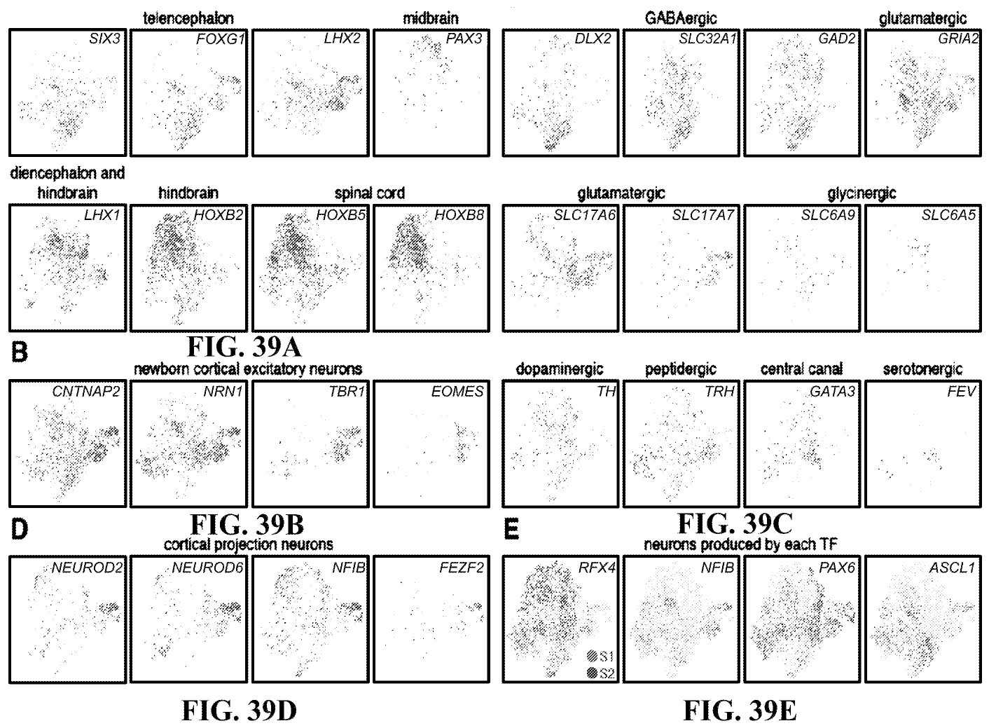

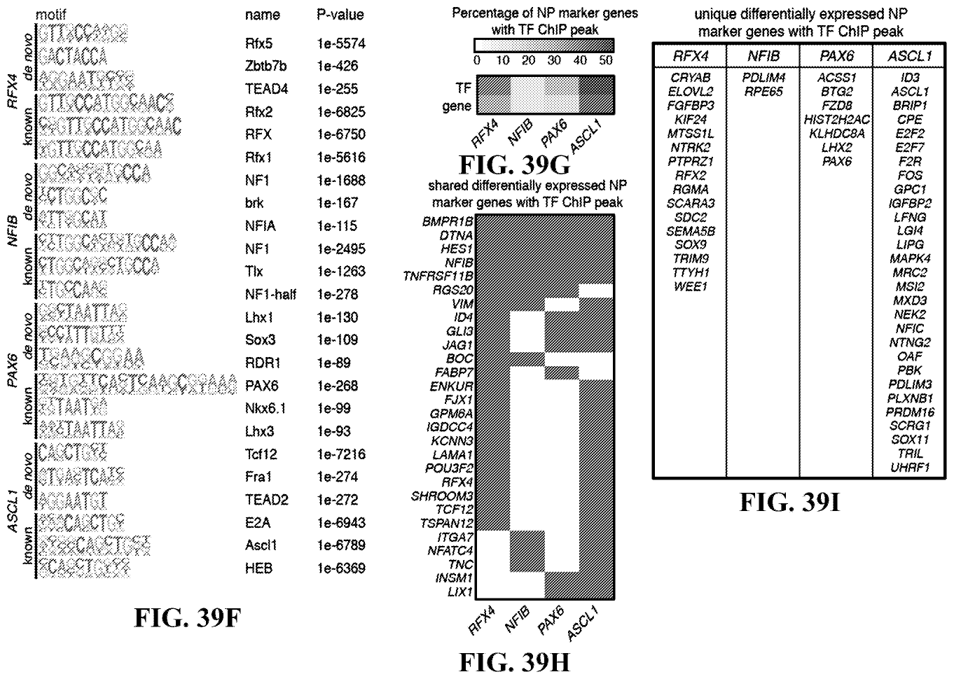

Applicants further characterized the cells spontaneously differentiated from iNPs produced by these four TFs using scRNA-seq. Cluster analysis of 53,113 cells revealed that the iNPs generated a broad range of cell types, such as cell types from the retina, CNS, epithelium, and neural crest (FIGS. 32A-C and Table 6). For the CNS, iNPs spontaneously produced different regionally-restricted progenitors, such as radial glia and dorsal neural progenitors, as well as neurons, astrocytes, and ependyma (FIGS. 32B and 32C). Applicants found that the spontaneously differentiated cell types were generally consistent between biological replicates of the same TF, except for those from RFX4-iNPs, and distinct between TFs (FIGS. 32D-F). RFX4-iNPs produced more CNS cell types; NFIB-iNPs produced more epithelium and neural crest cell types; PAX6-iNPs generated diverse cell types; and ASCL1-iNPs produced more retina cell types (FIGS. 32D-F). Further analysis of CNS neurons spontaneously differentiated from iNPs showed that the neurons expressed marker genes representative of diverse brain regions as well as neurotransmitters and included newborn cortical excitatory neurons and cortical projection neurons (FIGS. 39A-D). RFX4-iNPs generated diverse neurons, NFIB-iNPs produced more cortical projection and excitatory neurons, PAX6-iNPs produced more forebrain neurons, and ASCL1-iNPs generated more forebrain GABAergic neurons (FIG. 39E). Together, the spontaneous differentiation results show that four of the candidate TFs produce functional iNPs.

To better understand the transcriptional networks that lead to iNP production, Applicants profiled the four TFs using chromatin immunoprecipitation with sequencing (ChIP-seq). Motif analysis generated distinct motifs for each TF and suggested potential transcriptional coregulators, some of which have been found in previous studies (FIG. 39F) (Morotomi-Yano et al., 2002; Murre et al., 1989). Applicants identified candidate genes that could contribute to the potential mechanisms behind directed iNP differentiation by examining NP marker genes with TF ChIP-seq peaks that were also differentially expressed upon TF overexpression (FIGS. 39G-I and Table 8). In addition, Applicants found that each of the four TFs had ChIP-seq peaks that were proximal to its own promoter, indicating a positive feedback mechanism that contributes to the high expression levels required for driving differentiation (FIGS. 39H and 39I).

### Example 18—Combining RFX4 with Dual SMAD Inhibition Produces Homogenous iNPs

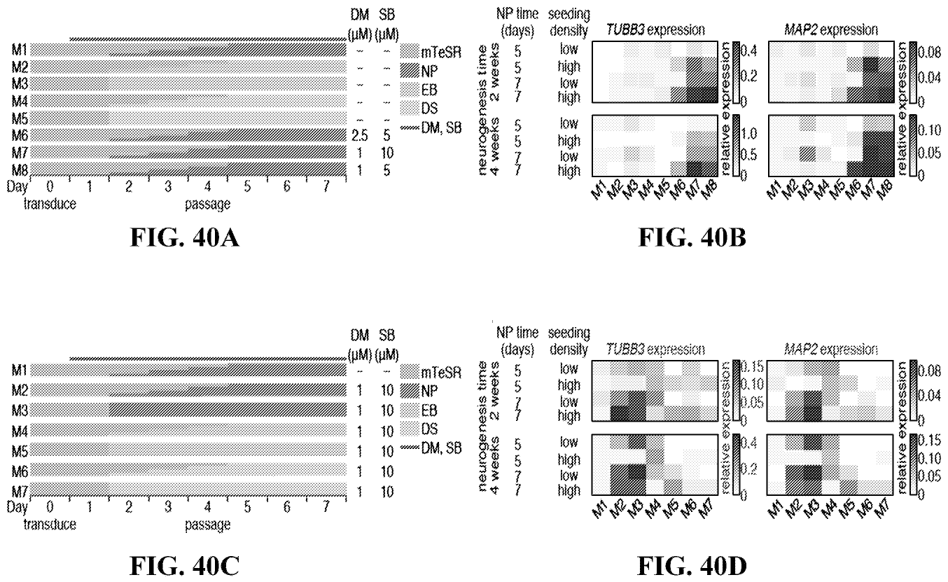

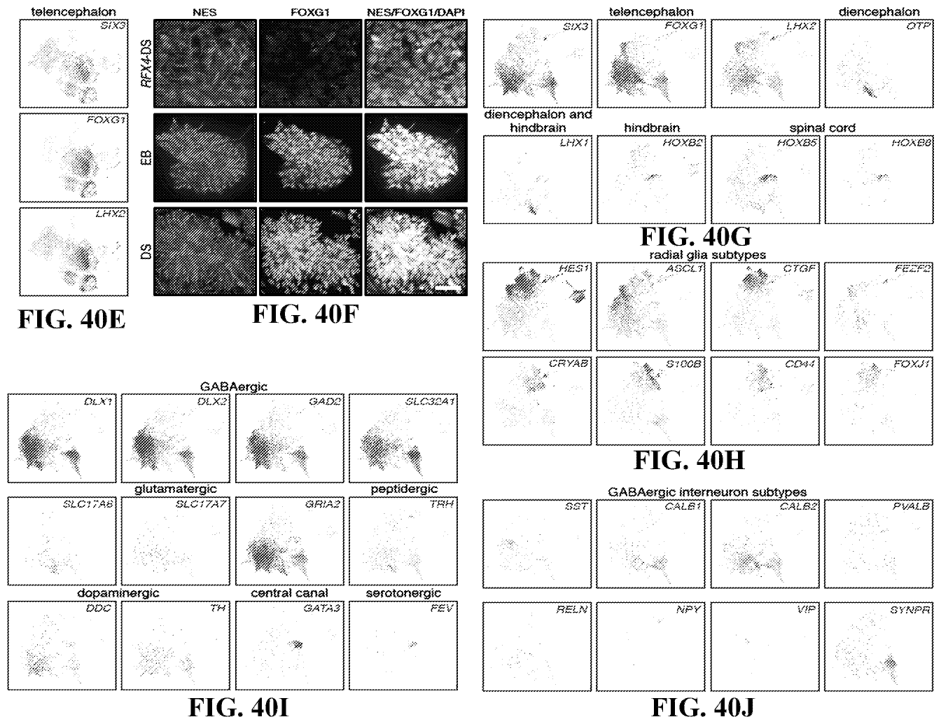

Next, Applicants sought to improve the consistency of RFX4-iNPs. Although RFX4-iNPs produced the highest proportion of CNS cell types, the iNPs were less consistent between biological replicates (FIGS. 32D-F). Applicants overexpressed RFX4 in H1 hESCs and tested transition from stem cell media to two alternative NP media used in the embryoid body (EB) (Schafer et al., 2019) and dual SMAD inhibition (DS) (Shi et al., 2012a) NP differentiation methods (FIG. 40A). Applicants also tested addition of dual SMAD inhibitors and two different NP induction times, 5 and 7 days (FIG. 40A). By spontaneously differentiating the iNPs and measuring expression of the neuronal marker genes TUBB3 and MAP2 as a heuristic for the proportion of iNPs that underwent neurogenesis, Applicants could identify conditions that promoted differentiation of CNS iNPs and increased homogeneity of the iNP population. Applicants found that combining RFX4 overexpression with dual SMAD inhibitors in the initial NP media for 7 days produced the most homogenous iNPs (FIGS. 40A-D).

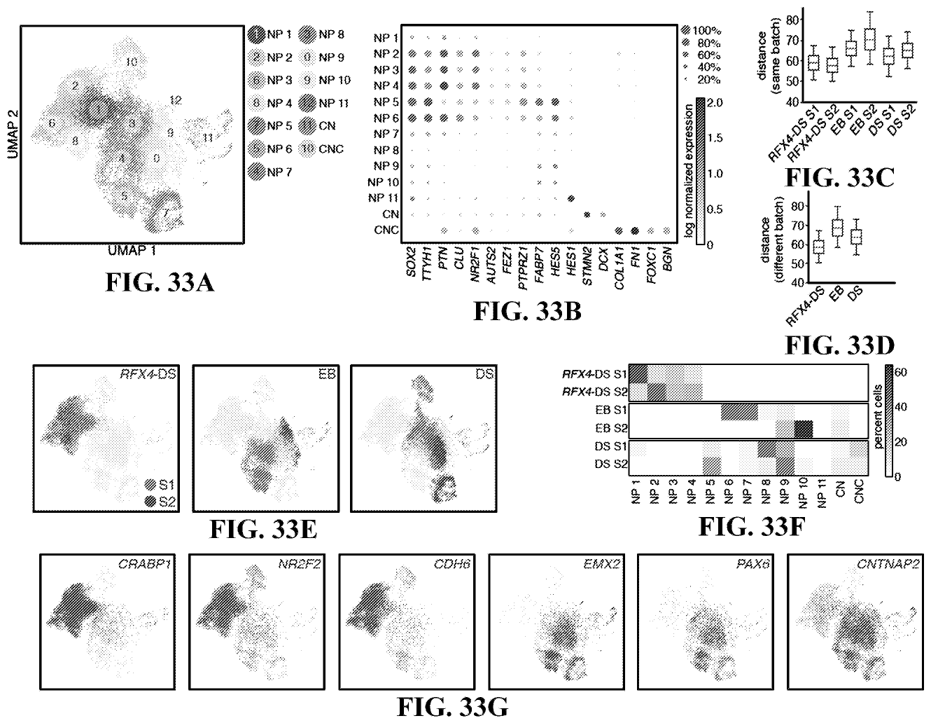

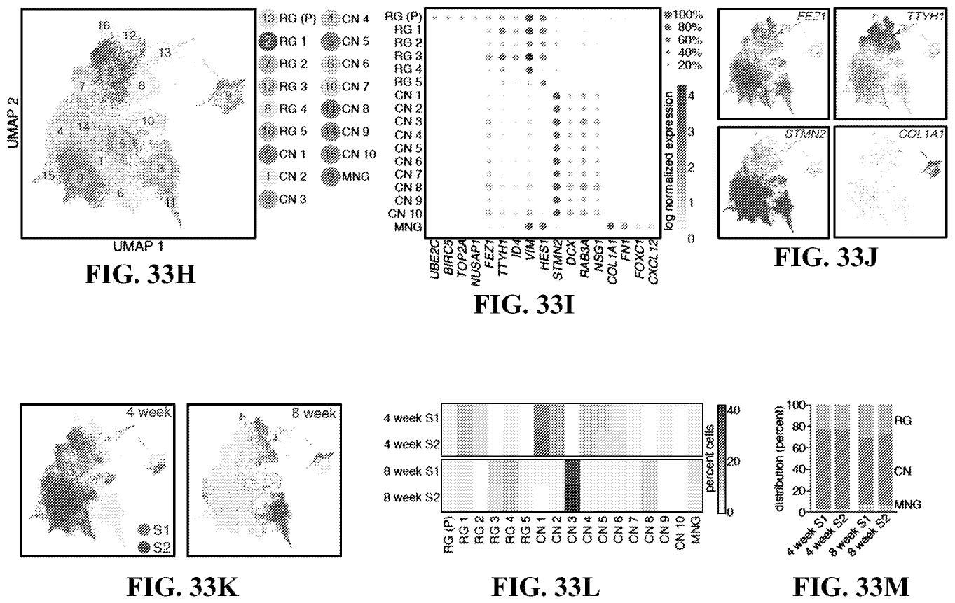

Applicants then compared iNPs generated by the optimized protocol, RFX4-DS, to those from two alternative NP differentiation methods that rely on EB (Schafer et al., 2019) and DS (Shi et al., 2012a). Applicants derived iNPs using the three differentiation methods in two batch replicates and performed scRNA-seq on 42,780 iNPs (15,211 RFX4-DS-iNPs, 11,148 EB-iNPs, and 16,421 DS-iNPs). Cluster analysis showed that, as expected, the majority of the cells were NPs (FIGS. 33A and 33B; Table 6). Applicants also observed immature neurons that have spontaneously differentiated from iNPs and cranial neural crest cells that were off-target products of NP differentiation (FIGS. 33A and 33B). Using distances between cells from the same batch replicate and cells from different batch replicates as metrics for intra- and inter-batch variability respectively, Applicants found that RFX4-DS-iNPs had lower intra- and inter-batch distances compared to EB- and DS-iNPs (FIGS. 33C and 33D). In addition, batch replicates of RFX4-DS-iNPs had more consistent percentages of cells that were grouped into each cluster than those of EB- and DS-iNPs, suggesting that the RFX4-DS protocol produces more consistent iNPs than alternative protocols (FIGS. 33E and 33F). All three protocols generated iNPs that expressed telencephalon markers such as SIX3 and LHX2, although RFX4-DS-iNPs did not express FOXG1, suggesting that there may be potential differences between RFX4-DS-iNPs and iNPs generated by existing methods that could contribute to differences in downstream cell types derived from iNPs (FIG. 40E). Applicants confirmed this observation by immunostaining iNPs for FOXG1 (FIG. 40F). Further analysis of genes that were differentially expressed between iNP differentiation methods showed that RFX4-DS-iNPs expressed higher levels of CRABP1, NR2F2, and CDH6, whereas EB- and DS-iNPs expressed EMX2, PAX6, and CNTNAP2 (FIG. 33G). These results indicate that RFX4-DS-iNPs may resemble NPs of the deep layer neocortex, rather than of the ventricular zone (Cadwell et al., 2019; Matsunaga et al., 2015).

To characterize the cells spontaneously differentiated from RFX4-DS-iNPs, Applicants performed scRNA-seq on 26,111 cells at 4 and 8 weeks of spontaneous differentiation. Cluster analysis showed that RFX4-DS-iNPs differentiated into predominantly CNS cell types, radial glia, and neurons, with a small subset differentiating into meningeal cells (FIGS. 33H-J and Table 6). At each differentiation time point, the spontaneously differentiated cell types were remarkably consistent between biological replicates (FIGS. 33K and 33L). RFX4-DS-iNPs produced 98% CNS cell types at 4 weeks and 94% at 8 weeks (FIG. 33M), suggesting that initially >98% of iNPs were capable of spontaneously differentiating into CNS cell types because differentiated neurons do not divide, unlike meningeal cells. Similar to RFX4-DS-iNPs, most of the radial glia differentiated from RFX4-DS-iNPs expressed telencephalon marker genes SIX3 and LHX2, but not FOXG1 (FIG. 40G). By contrast, differentiated neurons expressed all three marker genes (FIG. 40G). The radial glia were diverse, with some expressing markers indicative of more restricted precursors for astrocytes (CD44) and ependymal cells (FOXJ1; FIG. 40H). RFX4-DS-iNPs produced predominantly GABAergic neurons (GAD2 and SLC32A1) that expressed markers indicative of different GABAergic interneuron subtypes, such as SST, CALB1, CALB2, and PVALB (FIGS. 401 and 40J). The propensity for RFX4-DS-iNPs to spontaneously differentiate into GABAergic neurons, rather than glutamatergic neurons as previously shown for iNPs produced by alternative methods (Schafer et al., 2019; Shi et al., 2012b), may stem from initial differences observed between the iNPs (FIGS. 33G, 40E, and 40F). Specifically, RFX4-DS-iNPs expressed higher levels of NR2F2, a marker gene for cortical GABAergic interneurons originating from the ganglionic eminence and neocortex in the human fetal forebrain (Reinchisi et al., 2012). RFX4 ChIP-seq and bulk RNA-seq data further suggests that RFX4 directly regulates NR2F2, as RFX4 had a ChIP-seq peak within 5 kb of all four annotated transcriptional start sites of NR2F2 isoforms and RFX overexpression robustly upregulated expression of NR2F2 (Tables 7 and 8). Overall, the results suggest that RFX4 overexpression can be combined with dual SMAD inhibition to produce homogenous iNPs that spontaneously differentiate into GABAergic neurons.

### Example 19—RFX4-iNPs Accurately Model Effects of DYRK1A Perturbations on Neural Development

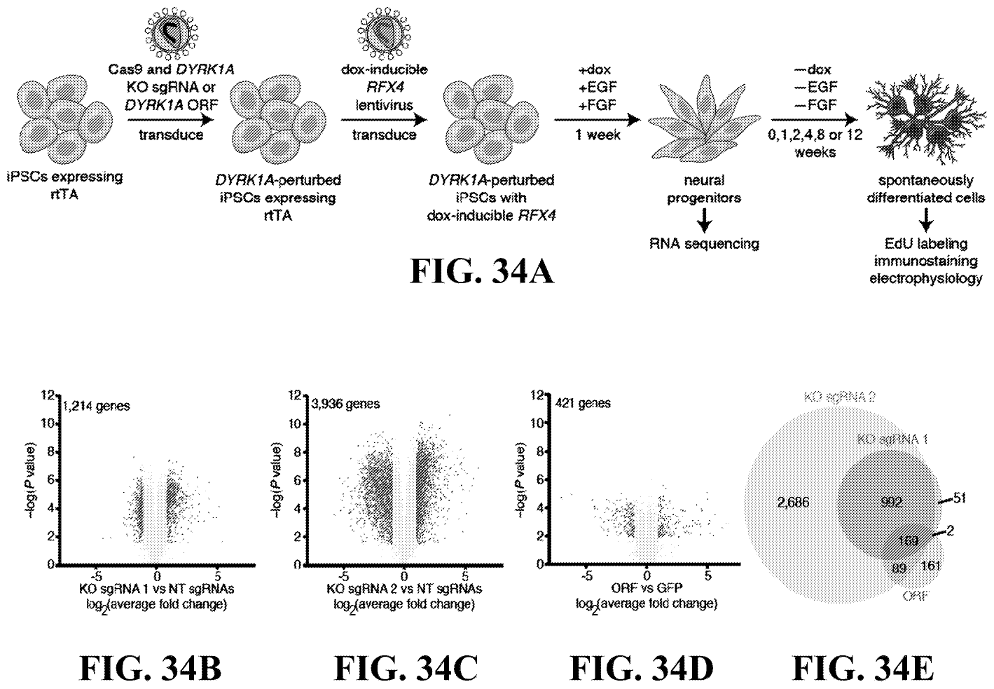

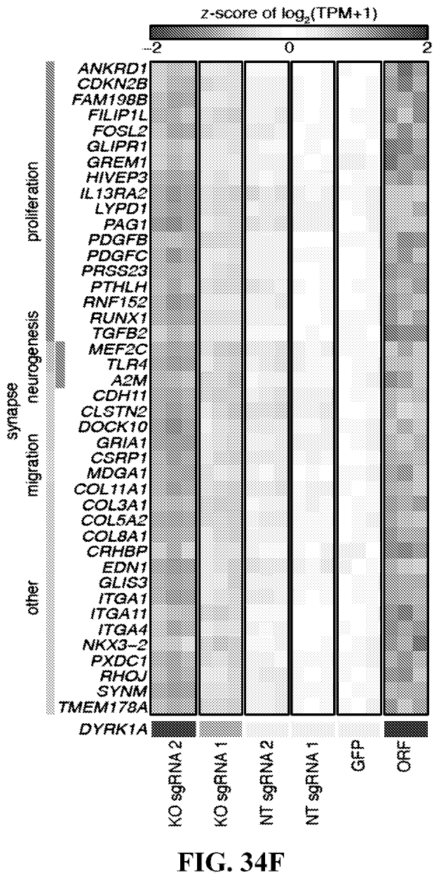

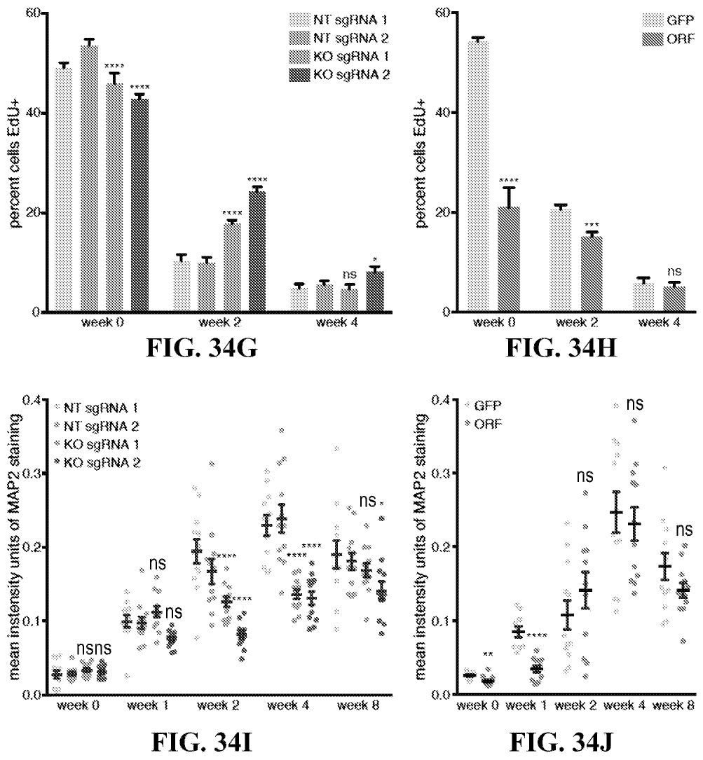

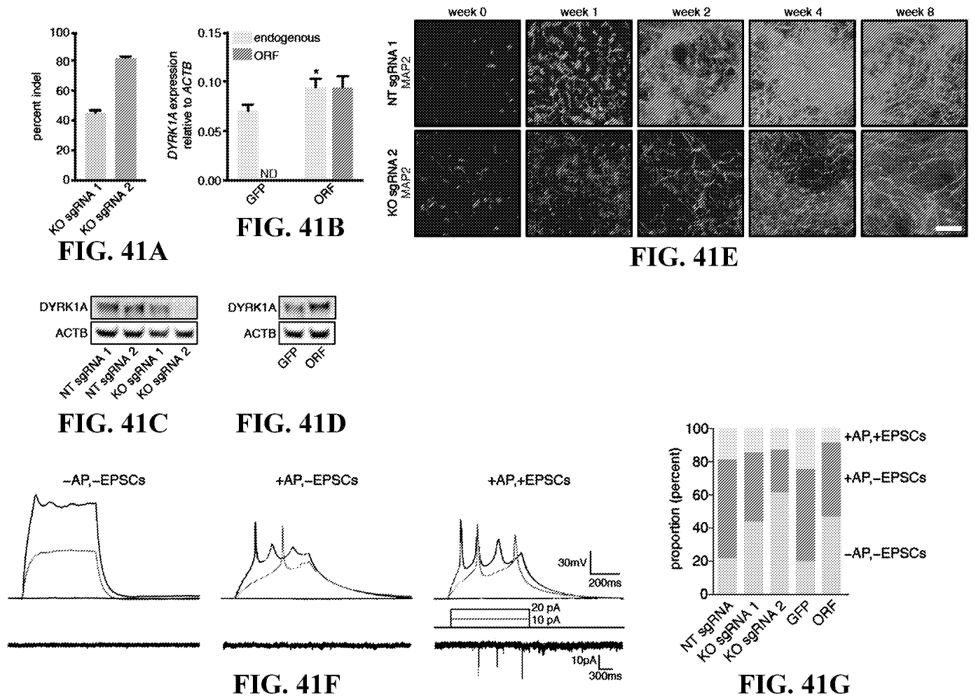

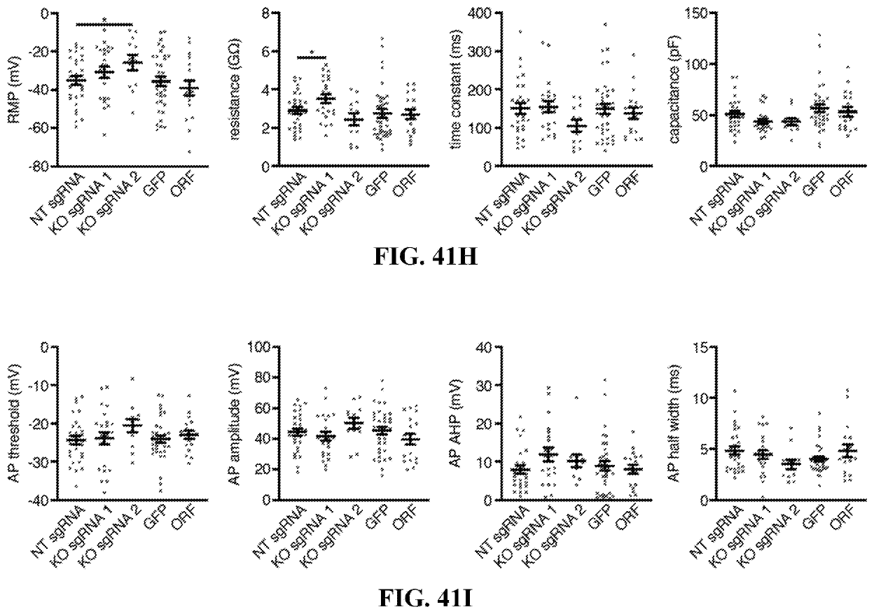

To explore the utility of the differentiation protocol Applicants developed, Applicants transiently overexpressed RFX4 to differentiate iPSC11a into iNPs to study the effects of DYRK1A perturbation on NPs during neural development (FIGS. 34A and 41A-D). DYRK1A knockout has been implicated in autism spectrum disorder (De Rubeis et al., 2014; Iossifov et al., 2014), whereas overexpression of DYRK1A has been linked to Down syndrome (Smith et al., 1997). Applicants characterized iNPs using bulk RNA-seq and identified 42 genes that were significantly differentially expressed in a DYRK1A dosage-dependent manner, some of which are known to be involved in cellular proliferation, neuronal migration, and synapse formation (FIGS. 34B-F; Table 7). Applicants spontaneously differentiated the RFX4-derived iNPs to profile the effects of DYRK1A perturbation on neurogenesis and neural development. DYRK1A knockout iNPs initially showed reduced proliferation, potentially due to toxicity of DNA double-strand breaks introduced by Cas9, but at weeks 2 and 4 of spontaneous differentiation, DYRK1A knockout iNPs showed significantly increased proportions of proliferating cells, indicating that more iNPs were actively dividing instead of undergoing neurogenesis (FIG. 34G). By contrast, DYRK1A overexpressing iNPs showed lower proportions of proliferating cells at weeks 0 and 2 (FIG. 34H). As increased iNP proliferation deters neurogenesis, Applicants immunostained spontaneously differentiating iNPs for expression of the neuronal marker MAP2. For the DYRK1A knockout iNPs, Applicants observed a significant reduction in neuronal MAP2 staining at weeks 2 and 4 (FIGS. 341 and 41E). For the DYRK1A overexpression iNPs, as there were fewer iNPs due to lower initial proliferation, Applicants observed significant reductions in neuronal MAP2 staining at weeks 0 and 1 (FIG. 34J).

Applicants further characterized neurons spontaneously differentiated from DYRK1A-perturbed iNPs using electrophysiology. Whole-cell patch-clamp recording of neurons after 12-14 weeks of spontaneous differentiation confirmed that neurons derived from unperturbed iNPs were electrophysiologically functional (FIGS. 41F and 41G). Both DYRK1A knockout and overexpression iNPs exhibited reduced proportions of neurons with properties indicative of maturation, such as presence of evoked action potentials and spontaneous excitatory postsynaptic activity (FIGS. 41F and 41G). In addition, neurons produced by DYRK1A knockout iNPs had higher resting membrane potential and membrane resistance (FIG. 41H). Applicants did not observe any significant differences in action potential properties (FIG. 41I). Together, these electrophysiology results suggest that neurons spontaneously differentiated from DYRK1A knockout and overexpression iNPs are less mature. The DYRK1A perturbation results are consistent with previous studies in other model systems (Fotaki et al., 2002; Hammerle et al., 2011; Park et al., 2010; Soppa et al., 2014; Yabut et al., 2010) and provide additional insight for how different DYRK1A expression levels can affect neural development. Thus, RFX4-iNPs can be used to model effects of perturbations on neural development and neurogenesis and may serve as a tractable system for studying complex neurological disorders.

### Example 20—Discussion

By screening TF ORFs, Applicants were able to identify four TFs that could individually differentiate hESCs and induced pluripotent stem cells into iNPs that resemble the morphology, transcriptome signature, and multipotency of NPs. Of the four candidate TFs, overexpression of RFX4, which has not been extensively studied in CNS development, resulted in the highest proportion of CNS cell types, highlighting the importance of performing large-scale, unbiased TF screens (Ashique et al., 2009; Blackshear et al., 2003). Combining RFX4 overexpression with dual SMAD inhibition produced homogenous iNPs that spontaneously differentiated into predominantly GABAergic neurons. Notably, the differentiation method produced iNPs within 7 days, compared to 11-16 days for existing differentiation methods, and is more scalable than the embryoid body method (Chambers et al., 2009; Schafer et al., 2019; Shi et al., 2012a; Zhang et al., 2001). By perturbing DYRK1A in iNPs to model neurodevelopmental disorders, Applicants found that DYRK1A modulates iNP proliferation to disrupt neurogenesis, confirming results from previous studies in other model systems (Fotaki et al., 2002; Hammerle et al., 2011; Park et al., 2010; Soppa et al., 2014; Yabut et al., 2010) and suggesting candidate genes that mediate the effect of DYRK1A on neural development.

Although Applicants focused here on 90 TF isoforms highly expressed in the target cell type (˜23% of TFs expressed in NPs and ˜2.5% of all TF isoforms), the accessibility and low-cost nature of the multiplexed screening approach lends itself to scalable extensions of the technology to additional cell types of interest. For some of these cell types, Applicants have recommended lists of marker genes and TFs based on published RNA-seq datasets (Table 9). Applicants have also provided code for aggregating gene lists from different datasets and selecting marker genes and a subset of TFs from the TF library for targeted screening (see Methods). Moreover, the approach may be applied to identify combinations of TFs by screening at a higher MOI to increase the probability of introducing more than one TF in the same cell. Iterative TF screens may also expand the landscape of cell types it is possible to generate with this platform. For instance, performing TF screens in iNPs for differentiation into neurons or glia may facilitate generation of mature cell types as iterative overexpression of TFs may mimic the natural developmental trajectory.

Beyond directed differentiation, TF screening enables identification of factors involved in cellular reprogramming (Takahashi and Yamanaka, 2006) and trans-differentiation (Pang et al., 2011; Song et al., 2012), as well as cancer progression (Darnell, 2002) and senescence (Campisi, 2001). The ORF barcoding approach allows for a variety of screening selection methods and could also be extended to pooled ORF screening of other protein families of interest. Future application of the multiplexed TF screening platform for cellular engineering has the potential to expand the number of available cellular models that will help elucidate complex regulatory mechanisms behind development and disease.

### Example 21—Methods for Examples 1-21

Sequences and cloning. The plasmids lentiMPHv2 (Addgene 89308) and lentiSAMv2 (Addgene 75112) were used for CRISPR activation. LentiCRISPRv2 (Addgene 52961) was used for CRISPR-Cas9 mediated homology-directed repair (HDR). The Puromycin resistance gene in lentiCRISPRv2 was replaced with Blasticidin resistance gene (Addgene 75112) for CRISPR-Cas9 knockout of DYRK1A. Single guide RNA (sgRNA) spacer sequences used in this study are listed in Table 10, and cloned into the respective vectors as previously described (Joung et al., 2017b). For spontaneous differentiation using a dox-inducible gene expression system, the plasmid pUltra-puro-RTTA3 (Addgene 58750) was used for rtTA. The EF1a promoter in pLX_TRC209 (Broad Genetic Perturbation Platform) was replaced with the pTight promoter (Addgene 31877). For DYRK1A overexpression, the codon-optimized DYRK1A sequence (NM_001396) was cloned into pLX_TRC209 (Broad Genetic Perturbation Platform) for expression under EF1a and the Hygromycin resistance gene was replaced with a Blasticidin resistance gene (Addgene 75112).

Cell culture and differentiation. HEK293FT cells (Thermo Fisher Scientific R70007) were maintained in high-glucose DMEM with GlutaMax and pyruvate (Thermo Fisher Scientific 10569010) supplemented with 10% fetal bovine serum (VWR 97068-085) and 1% penicillin/streptomycin (Thermo Fisher Scientific 15140122). Cells were passaged every other day at a ratio of 1:4 or 1:5 using TrypLE Express (Thermo Fisher Scientific 12604021).

Unless otherwise specified, human embryonic stem cells (hESCs) used in these experiments were from the HUES66 cell line (Harvard Stem Cell Institute iPS Core Facility). Other stem cell lines used in this study include human induced pluripotent stem cell (iPSC) 11a (gift from the Arlotta laboratory, Harvard University) and hESC H1 (WiCell). hESCs and iPSCs were maintained in cell culture dishes coated with 1% Geltrex membrane matrix (Thermo Fisher Scientific A1413202) in mTeSR1 medium (STEMCELL Technologies 85850). For routine maintenance, stem cells were passaged 1:10-1:20 using ReLeSR (STEMCELL Technologies 05873) and seeded in mTeSR with 10 μM ROCK Inhibitor Y27632 (Enzo Life Sciences ALX-270-333-M025). For lentivirus transduction and differentiation, cells were dissociated using Accutase (STEMCELL Technologies 07920). All stem cells were maintained below passage 30 and confirmed to be karyotypically normal and negative for mycoplasma within 5 passages before differentiation.

During neuronal differentiation, stem cell media was incrementally shifted towards neuronal media, consisting of Neurobasal medium (Thermo Fisher Scientific 21103049) supplemented with B-27 (Thermo Fisher Scientific 17504044), GlutaMAX (Thermo Fisher Scientific 35050061), and Normocin (Invivogen ant-nr-1). 1 day after the start of differentiation (day 1), media was changed to stem cell media with the appropriate antibiotic. Antibiotic was included in the media for a total of 5 days of selection. On day 2, media was changed to 75% stem cell media and 25% neuronal media. On day 3, media was changed to 50% stem cell media and 50% neuronal media. On day 4, media was changed to 25% stem cell media and 75% neuronal media. On day 5, media was changed to neuronal media.

During TF-driven neural progenitor (NP) differentiation, stem cell media was gradually shifted towards NP media, consisting of DMEM/F-12 with HEPES (Thermo Fisher Scientific 11330057) supplemented with B-27 (Thermo Fisher Scientific 17504044), 20 ng/ml EGF (MilliporeSigma E9644), 20 ng/mL bFGF (STEMCELL Technologies 78003), 2 ug/ml heparin (STEMCELL Technologies 07980), and Normocin (Invivogen ant-nr-1). Similar to neuronal differentiation, stem cell media was shifted by increasing the proportion of NP media 25% incrementally from day 2 to day 5. Cells were passaged at day 4 when selected with the appropriate antibiotic. For spontaneous differentiation, 2 μg/mL doxycycline (MilliporeSigma D9891) was added to the media starting from day 0 for 7 days. After 7 days, cells were maintained in NP media for 3 days before media was changed to differentiation media, which had the same components as NP media but without EGF and bFGF. During spontaneous differentiation, 40-60% of differentiation media was refreshed every other day.

For comparison to other NP differentiation methods, embryoid body (EB) (Schafer et al., 2019) and dual SMAD inhibition (DS) (Shi et al., 2012a) methods were used to differentiate hESCs into NP as previously described. To provide the best comparison between the methods, the differentiation timelines for the three methods were aligned such that the iNP differentiation ended around the same time. The iNPs produced by the three methods were dissociated for scRNA-seq at the same time. During the RFX4-iNP protocol optimization, base media from the DS and EB protocols were tested. DS media is a 1:1 mix of N-2 and B-27-containing media. N-2 medium consists of DMEM/F12 with HEPES (Thermo Fisher Scientific 11330057) supplemented with N-2 (Thermo Fisher Scientific 17502048), 5 μg/mL insulin (Millipore Sigma 19278), 100 μM nonessential amino acids (Thermo Fisher Scientific 11140050), 100 M 2-mercaptoethanol (Millipore Sigma M6250), and Normocin (Invivogen ant-nr-1). B-27 medium is the same as the neuronal medium described above. EB media consists of DMEM/F12 with HEPES (Thermo Fisher Scientific 11330057) supplemented with N-2 (Thermo Fisher Scientific 17502048), B27 minus vitamin A (Thermo Fisher Scientific 12587010), and Normocin (Invivogen ant-nr-1). SMAD inhibitors dorsomorphin (Millipore Sigma P5499) and SB-431542 (R&D Systems 1614) were added where indicated.

Lentivirus production. HEK293FT cells (Thermo Fisher Scientific R70007) were cultured as described above. 1 day prior to transfection, cells were seeded at ˜40% confluency in T25, T75, or T225 flasks (Thermo Fisher Scientific 156367, 156499, or 159934). Cells were transfected the next day at ˜90-99% confluency. For each T25 flask, 3.4 μg of plasmid containing the vector of interest, 2.6 μg of psPAX2 (Addgene 12260), and 1.7 μg of pMD2.G (Addgene 12259) were transfected using 17.5 μL of Lipofectamine 3000 (Thermo Fisher Scientific L3000150), 15 μL of P3000 Enhancer (Thermo Fisher Scientific L3000150), and 1.25 mL of Opti-MEM (Thermo Fisher Scientific 31985070). Transfection parameters were scaled up linearly with flask area for T75 and T225 flasks. Media was changed 5 h after transfection. Virus supernatant was harvested 48 h post-transfection, filtered with a 0.45 μm PVDF filter (MilliporeSigma SLHV013SL), aliquoted, and stored at −80° C.

Lentivirus transduction. For transduction, 3×106 hESCs or iPSCs were seeded in 10-cm cell culture dishes with 10 μM ROCK Inhibitor Y27632 (Enzo Life Sciences ALX-270-333-M025) and an appropriate volume of lentivirus in mTeSR. After 24 h, media was refreshed with the appropriate antibiotic. For 5 days, media with the appropriate antibiotic was refreshed every day, and cells were passaged after 3 days of selection. Concentrations for selection agents were determined using a kill curve: 150 μg/mL Hygromycin (Thermo Fisher Scientific 10687010), 3 μg/mL Blasticidin (Thermo Fisher Scientific A1113903), and 1 μg/mL Puromycin (Thermo Fisher A1113803). Lentiviral titers were calculated by transducing cells with 5 different volumes of lentivirus and determining viability after a complete selection of 3 days (Joung et al., 2017b).

qPCR quantification of transcript expression. Cells were seeded in 96-well plates and grown to 60-90% confluency before RNA was reverse transcribed for qPCR as described previously (Joung et al., 2017b). TaqMan qPCR was performed with custom or readymade probes (Tables 11 and 12). Significance testing was performed using Student's t-test.

Western blot. Protein lysates were harvested with RIPA lysis buffer (Cell Signaling Technologies 9806S) containing protease inhibitor cocktail (MilliporeSigma 05892791001). Samples were standardized for protein concentration using the Pierce BCA protein assay (VWR 23227), and 20 μg or 40 μg of the samples were incubated at 70° C. for 10 mins under reducing conditions. After denaturation, samples were separated by Bolt 4-12% Bis-Tris Plus Gels (Thermo Fisher Scientific NW04125BOX) and transferred onto a PVDF membrane using iBlot Transfer Stacks (Thermo Fisher Scientific IB401001).

For NEUROD1 and V5, blots were blocked with Odyssey Blocking Buffer (TBS; LiCOr 927-50000) for 1 h at room temperature. Blots were then probed with different primary antibodies [anti-NEUROD1 (Abcam ab60704, 1:1,000 dilution), anti-GAPDH (Cell Signaling Technologies 2118L, 1:1,000 dilution), anti-V5 (Cell Signaling Technologies 13202S, 1:1,000 dilution), anti-ACTB (MilliporeSigma A5441, 1:5,000 dilution)] in Odyssey Blocking Buffer overnight at 4° C. Blots were washed with TBST before incubation with secondary antibodies IRDye 680RD Donkey anti-Mouse IgG (LiCOr 925-68072) and IRDye 800CW Donkey anti-Rabbit IgG (LiCOr 925-32213) at 1:20,000 dilution in Odyssey Blocking Buffer for 1 h at room temperature. Blots were washed with TBST and imaged using the Odyssey CLx (LiCOr).

For DYRK1A, blots were blocked with 5% BLOT-QuickBlocker (G Biosciences 786-011) in TBST for 1 h at room temperature. Blots were then probed with different primary antibodies [anti-DYRK1A (Novus Biologicals H00001859-M01, 1:250 dilution) or anti-ACTB (Cell Signaling Technologies 4967L, 1:1,000 dilution)] in 2.5% BLOT-QuickBlocker (G Biosciences 786-011) in TBST overnight at 4° C. Blots were washed with TBST before incubation with secondary antibodies anti-mouse IgG, HRP-linked antibody (Cell Signaling Technologies 7076S) and anti-rabbit IgG, HRP-linked antibody (Cell Signaling Technologies 7074S) at 1:5,000 dilution in 2.5% BLOT-QuickBlocker (G Biosciences 786-011) in TBST for 1 h at room temperature. Blots were washed with TBST and imaged using the Pierce ECL Western Blotting Substrate (Thermo Fisher Scientific 32209) on the ChemiDox XRS+ (Bio-Rad).

Immunofluorescence and imaging. Cells were cultured on poly-D-lysine/laminin coated glass coverslips (VWR 354087) in 24-well plates as described above. Prior to staining, cells were washed with 1 mL PBS and fixed with 4% paraformaldehyde (VWR 15710) in PBS for 30 mins at room temperature. Cells were washed with PBS and blocked in PBS with 2.5% goat serum (Cell Signaling Technologies 5425S) and 0.1% Triton X-100 (MilliporeSigma 93443) for 1 h at room temperature. Cells were then stained with different primary antibodies [anti-MAP2 (MilliporeSigma M1406, 1:500 dilution), anti-PAX6 (Abcam ab5790, 1:500 dilution), anti-Nestin (MilliporeSigma MAB5326, 1:200 dilution), anti-VIM (Proteintech 10366-1-AP, 1:200 dilution), anti-GFAP (Abcam ab4674, 1:500 dilution), anti-NG2 (MilliporeSigma AB5320, 1:200 dilution), anti-PDGFRA (Cell Signaling Technologies 3164S, 1:200 dilution), or anti-FOXG1 (Abcam ab18259, 1:500 dilution] in PBS with 1.25% goat serum (Cell Signaling Technologies 5425S) and 0.1% Triton X-100 (MilliporeSigma 93443) overnight at 4° C. Cells were washed in PBS with 0.1% Triton X-100 (MilliporeSigma 93443) before staining with the appropriate secondary antibodies [goat anti-mouse IgG (Alexa Fluor 568, Thermo Fisher Scientific A-11031, 1:1,000 dilution), goat anti-chicken IgY (Alexa Fluor 488, Thermo Fisher Scientific A-11039, 1:1,000 dilution), goat anti-rabbit IgG (Alexa Fluor 647, Thermo Fisher Scientific A-21244, 1:1,000 dilution), or goat anti-rabbit IgG (Alexa Fluor 488, Thermo Fisher Scientific A-11008, 1:1,000 dilution)] in PBS with 1.25% goat serum (Cell Signaling Technologies 5425S) and 0.1% Triton X-100 (MilliporeSigma 93443) for 1 h at room temperature. Cells were washed in PBS with 0.1% Triton X-100 (MilliporeSigma 93443), mounted onto slides using ProLong Gold Antifade Mountant with DAPI (Thermo Fisher Scientific P36941), and nail polished (VWR 100491-940). Immunostained coverslips were imaged on a Zeiss Axio Observer with a Hamatsu Camera using a Plan-Apochromat 20x objective and a 1.6× Optovar.

Image quantification. Images were taken from randomly selected regions using fixed exposure times. The MeasureImageIntensity module in CellProfiler 3.1.8 was used to analyze grayscale 577 nm images (MAP2) for mean intensity units. For induced neurons, mean intensity units were normalized by the number of nuclei in each image. The IdentifyPrimaryObjects module in CellProfiler was used to identify and count nuclei in the grayscale 353 nm (DAPI) images with the following settings modified from default: Typical diameter of objects, in pixel units (Min, Max): 25, 70; Threshold strategy: Adaptive; Threshold smoothing scale: 1.5; Lower and upper bounds on threshold: 0.06, 1.0. Significance testing was performed using Student's t-test.

Design and cloning of TF ORF libraries. The barcoded human TF library (MORF) consisted of 1,836 genes that were selected based on AnimalTFDB (Zhang et al., 2015) and Uniprot (UniProt, 2015) annotations and included histone modifiers. The library included 3,548 isoforms that overlapped between RefSeq and Gencode annotations, as well as 2 control vectors expressing GFP and mCherry. 593 of the 3,548 isoforms were obtained from the Broad Genomic Perturbation Platform and sequence verified. Table 3 lists the sequences of TFs in MORF.

To design a targeted TF ORF library for NP differentiation, single-cell or bulk RNA-seq datasets of human or mouse radial glia, neural stem cells, differentiated neural progenitors from 2D cultures or brain organoids, and fetal astrocytes were used to select TFs that were shown to be specifically expressed in these cell types (Camp et al., 2015; Johnson et al., 2015; Llorens-Bobadilla et al., 2015; Pollen et al., 2015; Shin et al., 2015; Thomsen et al., 2016; Wu et al., 2010; Zhang et al., 2016). TFs that were identified in 2 or more datasets (out of 8) were included in the library. Then, bulk RNA-seq data of human fetal astrocytes (Zhang et al., 2016) was used to identify TF isoforms annotated in RefSeq that comprised >25% of the TF gene transcripts. These criteria selected 90 TF isoforms covering 70 TF genes (Table 1).

TF ORF isoforms that were not available from the Broad Genomic Perturbation Platform were synthesized with 24-bp barcodes (Genewiz) and cloned in an arrayed format into pLX_TRC317 (MORF; Broad Genetic Perturbation Platform) or pLX_TRC209 (targeted NP library; Broad Genetic Perturbation Platform) for expression under the EF1a promoter. Barcodes for each TF were selected to have a Hamming distance of at least 3 compared to all other barcodes.

Reporter cell line screen. To generate reporter cell lines, EGFP from pLX_TRC209 (Broad Genetic Perturbation Platform) followed by a T2A (GGCAGTGGAGAGGGCAGAGGAAGTCTGCTAACATGCGGTGACGTCGAGGAGAA TCCTGGCCCA (SEQ ID NO: 10809)) self-cleaving peptide was inserted at the N-terminus of endogenous SLC1A3 and VIM genomic sequences. Clonal reporter cell lines were generated using CRISPR-Cas9 mediated HDR. To construct the HDR plasmids for each gene, the HDR templates that consisted of the 850-1,000 bp genomic regions flanking the sgRNA cleavage sites were PCR amplified from HUES66 genomic DNA using KAPA HiFi HotStart Readymix (KAPA Biosystems KK2602). Then EGFP-T2A flanked by HDR templates were cloned into pUC19 (Addgene 50005). HUES66 cells were nucleofected with 10 μg of sgRNA and Cas9 plasmid (Addgene 52961) and 6 μg of HDR plasmid using the P3 Primary Cell 4D-Nucleofector X Kit (Lonza V4XP-3024) according to the manufacturer's instructions. Cells were then seeded sparsely (2 electroporation reactions per 10-cm cell culture dish) to form single-cell clones. After 18 h, cells were selected for Cas9 expression with 0.5 μg/mL Puromycin for 2 days and expanded until colonies can be picked (˜1 week).

Cell colonies were detached by replacing the media with PBS and incubating at room temperature for 15 mins. Each cell colony was removed from the Petri dish using a 200 μL pipette tip and transferred a well in a 96-well plate for expansion. Clones with EGFP insertions were identified by 2-round PCR amplification (Table 13), first with primers amplifying outside of the HDR template (HDR Fwd 1 and HDR Rev, 15 cycles) and then with primers amplifying the region of insertion (HDR Fwd 2 and HDR Rev, 15 cycles) to avoid detecting the HDR template plasmid as a false positive. Products were run on a gel to identify clones with insertions and Sanger sequencing confirmed that EGFP had been inserted at the intended site without mutations. For each reporter cell line, 3 clones with EGFP inserted into one of the two alleles were selected for further expansion and characterization.

For TF ORF screening using reporter hESC lines, SLCIA3 or VIM reporter HUES66 cell lines were transduced with the pooled TF ORF library at MOI <0.3 and differentiated into iNPs as described above. After 7 days of differentiation, 5-10×106 cells were sorted for EGFP expression using the Sony SH800S Cell Sorter. For each clonal line, the percentage of cells sorted for the control condition was matched to those expressing EGFP (˜15-20%). After sorting, TF barcodes from each population were amplified (Table 13) and deep-sequenced on the Illumina MiSeq platform as previously described (>0.5 million reads per cell population) (Joung et al., 2017b). NGS reads that perfectly matched each barcode were counted and normalized to the total number of perfectly matched NGS reads for each condition. Enrichment of each TF was calculated as the normalized barcode count in the high population divided by the count in the low population.

Flow-FISH screen. For TF ORF screening using flow-FISH, HUES66 cells were transduced with the pooled TF ORF library at MOI <0.3 and differentiated into iNPs as described above. After 7 days of differentiation, cells were labeled with the appropriate FISH probes (Table 14) using the PrimeFlow RNA assay kit (Thermo Fisher Scientific 88-18005-204) with 20 million cells in 4 reactions per biological replicate. FISH probes targeting transcripts with similar expression levels were pooled together. Once the cells were labeled, the entire cell population was sorted for high or low fluorescence (15% of cells per bin), indicating an aggregate expression level of the transcripts labeled with the pooled FISH probes for the particular wavelength. After sorting, TF barcodes from each population were amplified (Table 13) using a modified ChIP reverse cross-linking protocol as described previously (Fulco et al., 2019) and deep-sequenced on the Illumina NextSeq platform (>4 million reads per cell population). Enrichment of each TF was calculated as described above for the reporter cell line screen.

Single-cell RNA sequencing (scRNA-seq) and data analysis. Cells were dissociated with Accutase (STEMCELL Technologies 07920) for 10 mins (NP) or 50 mins (spontaneously differentiated cells) at 37° C. and filtered using a 70 μm cell strainer (MilliporeSigma CLS431751) to obtain single cells. Cells were resuspended in PBS containing 0.04% BSA, counted, and loaded in the 10× Genomics Chromium Controller. 10,000 cells were used as input for each channel of a 10× Chromium Chip. For cells from the scRNA-seq pooled screen and spontaneous differentiation of four candidate TFs, scRNA-seq libraries were prepared using the Chromium Single Cell 3′ Library & Gel Bead Kit v2 (10× Genomics 120237) according to the manufacturer's instructions. Libraries were sequenced on the NextSeq platform, aiming for a minimum coverage of 20,000 reads per single cell (paired-end; read 1: 26 cycles; i7 index: 8 cycles, i5 index: 0 cycles; read 2: 55 cycles). For cells from the NP method comparison and spontaneous differentiation of RFX4-DS-iNPs, scRNA-seq libraries were prepared using the Chromium Single Cell 3′ Library & Gel Bead Kit v3 (10x Genomics 1000075) and sequenced on the HiSeq X platform (paired-end; read 1: 28 cycles; i7 index: 8 cycles, i5 index: 0 cycles; read 2: 96 cycles).

Sequencing data were aligned and quantified using the Cell Ranger Single-Cell Software Suite v3.1.0 (10× Genomics) (Zheng et al., 2017) against the GRCh38 human reference genome provided by Cell Ranger. The Python package Scanpy v1.4.4 (Wolf et al., 2018) was used to cluster and visualize cells. Cells with 400-7,000 detected genes and less than 5% total mitochondrial gene expression were retained for analysis. Genes that were detected in fewer than 3 cells were removed. Scanpy was used to log normalize, scale, and center the data and unwanted variation was removed by regressing out the number of UMIs and percent mitochondrial reads. Next, highly variable genes were identified and used as input for dimensionality reduction via principal component analysis (PCA). The resulting principal components were then used to cluster the cells, which were visualized using Uniform manifold approximation and projection (UMAP). Clusters were identified using Louvain by fitting the top 50 principal components to compute a neighborhood graph of observations with local neighborhood number of 20 using the scanpy.pp.neighbors function. Cells were then clustered into subgroups using the Louvain algorithm implemented as the scanpy.tl.louvain function. Cluster marker genes and associated p-values were identified using the scanpy.tl.rank_gene_groups function.

For scRNA-seq analysis of the pooled 90 TF screen for NP differentiation, distance between cells with different TF perturbations was calculated using the scipy.spatial.distance.cdist function from the SciPy Python library. For each TF perturbation, the pairwise distance between cells with the TF perturbation and cells without the TF perturbation was calculated and the median of the distances was determined. The 939 highly variable genes were used in the distance calculation. To identify TFs that produced transcriptome profiles similar to radial glia from human fetal cortex or brain organoid, TF scRNA-seq signatures were correlated to available scRNA-seq datasets (Nowakowski et al., 2017; Pollen et al., 2015; Quadrato et al., 2017). The 218 most variable genes in the scRNA-seq data, which were identified using the scanpy.pp.highly_variable_genes function with the parameters “min_mean=0.075, max_mean=8 and min_disp=1.5”, were used for the correlation analysis. The Spearman correlations between expression of these genes in each TF-perturbed single cell and the average expression in radial glia scRNA-seq from human fetal cortex or organoid were calculated. Then, the average correlation of each TF was determined by taking the average of the corresponding TF-perturbed single cell correlations. Candidate TFs were ranked based on the z-score of the average correlation across all datasets. For comparing TF transcriptome signatures to other cell types from the mouse organogenesis cell atlas (Cao et al., 2019), average expression of the top 30 marker genes (ranked by p-value) for each cell type was used to assess similarity. The z-score of the average marker gene expression for cells perturbed by each TF was used to identify TF perturbations that were most similar to each cell type.

For determining consistency within batch replicates of different iNP differentiation methods, the cluster of spontaneously differentiated neurons was excluded from the analysis. Distance between cells within the same batch replicate was calculated using the scipy.spatial.distance.pdist function from the SciPy Python library. The 2,305 highly variable genes were used in the distance calculation. For determining consistency between batch replicates, distance between cells in different batch replicates of the same method was calculated using the scipy.spatial.distance.cdist function.

ScRNA-seq screen. For TF ORF screening using scRNA-seq, HUES66 cells were transduced with the pooled TF ORF library at MOI <0.3 and differentiated into iNPs. Then, iNPs were dissociated for scRNA-seq analysis as described above. To pair TF barcodes with cell barcodes, TF and cell barcodes were PCR amplified from cDNA retained following the whole transcriptome amplification step of the 10× Genomics scRNA-seq library preparation protocol (Table 13). The resulting amplicon was sequenced on the Illumina NextSeq platform, aiming for a minimum coverage of 20,000 reads per single cell (paired-end; read 1: 16 cycles; read 2: 72 cycles). For each cell, the TF whose corresponding barcode had the highest number of perfectly matching NGS reads was paired with the cell if the TF barcode had at least 2 reads and >25% more reads than the second highest TF. Otherwise, the cell was excluded from the scRNA-seq analysis.

Arrayed screen. For TF ORF screening in an arrayed format, individual TF ORF isoforms were packaged into lentivirus as described above. Cells were transduced at MOI <0.5 by seeding 1.6×104 cells in 96-well plates and adding the appropriate volume of lentivirus. Cells were differentiated into NP and harvested for qPCR at 7 days after transduction as described above.

Bulk RNA sequencing (RNA-seq) and data analysis. RNA from cells plated in 24-well plates and grown to 60-90% confluency was harvested using the RNeasy Plus Mini Kit (Qiagen 74134). RNA-seq libraries were prepared using NEBNext Ultra RNA Library Prep Kit for Illumina (NEB E7530S) and deep sequenced on the Illumina NextSeq platform (>9 million reads per biological replicate). Bowtie(Langmead et al., 2009) index was created based on the human hg38 UCSC genome and RefSeq transcriptome. Next, RSEM v1.3.1 (Li and Dewey, 2011) was run with command line options “--estimate-rspd --bowtie-chunkmbs 512 --paired-end” to align paired-end reads directly to this index using Bowtie and estimate expression levels in transcripts per million (TPM) based on the alignments.

To correlate TF ORF RNA-seq signatures to those from human fetal cortex or brain organoid (Nowakowski et al., 2017; Pollen et al., 2015; Quadrato et al., 2017), transcript measurements from each available dataset were converted to TPM. For each cell type, TPM measurements from single cells were averaged to obtain average TPM values of genes for the cell type. The top 2,000 genes that had the highest fold change between the TF ORF expression condition compared to the GFP control condition (stem cells overexpressing GFP that were cultured in mTeSR1 stem cell media) were used to define the TF ORF RNA-seq signature. Expression of these genes in TPM was used to calculate the Pearson correlation between the TF ORF and the cell type of interest from available datasets.

To identify genes that were differentially expressed as a result of TF ORF expression, RSEM's TPM estimates for each transcript were transformed to log-space by taking log 2(TPM+1). Transcripts were considered detected if their transformed expression level was equal to or above 1 (in log 2(TPM+1) scale). All genes detected in at least three libraries were used to find differentially expressed genes. The Student's t-test was performed on the TF ORF overexpression condition against GFP control condition. Only genes that were significant (p-value pass 0.05 FDR correction) were reported.

For analysis of transcriptome changes as a result of DYRK1A perturbation, transcripts were considered detected if the average TPM of either the perturbed or control conditions was greater than 1. In the DYRK1A knockout perturbations, the Student's t-test was performed on the DYRK1A-targeting sgRNA condition against both non-targeting sgRNA conditions. In the DYRK1A overexpression perturbation, the Student's t-test was performed on the DYRK1A ORF condition against the GFP control condition. Volcano plots showed genes that had p-value pass 0.01 FDR correction with fold change that was greater or less than 1. The heat map of genes with DYRK1A dosage-dependent expression changes showed genes that had p-value pass 0.05 FDR correction.

Chromatin immunoprecipitation with sequencing (ChIP-seq). Cells were plated in 10-cm cell culture dishes and grown to 60-80% confluency. For each condition, two biological replicates were harvested for ChIP-seq. Formaldehyde (MilliporeSigma 252549) was added directly to the growth media for a final concentration of 1% and cells were incubated at 37° C. for 10 mins to initiate chromatin fixation. Fixation was quenched by adding 2.5 M glycine (MilliporeSigma G7126) in PBS for a final concentration of 125 mM glycine and incubated at room temperature for 5 mins. Cells were then washed with ice-cold PBS, scraped, and pelleted at 1,000×g for 5 mins.

Cell pellets were prepared for ChIP-seq using the Epigenomics Alternative Mag Bead ChIP Protocol v2.0 (Consortium, 2004). Briefly, cell pellets were resuspended in 100 μL of lysis buffer (1% SDS, 10 mM EDTA, 50 mM Tris-HCL pH 8.1) containing protease inhibitor cocktail (MilliporeSigma 05892791001) and incubated for 10 mins at 4° C. Then 400 μL of dilution buffer (0.01% SDS, 1.1% Triton X-100, 1.2 mM EDTA, 16.7 mM Tris-HCl pH 8.1, and 167 mM NaCl) containing protease inhibitor cocktail (MilliporeSigma 05892791001) was added. Samples were pulse sonicated with 2 rounds of 10 mins (30s on-off cycles, high frequency) in a rotating water bath sonicator (Diagenode Bioruptor) with 5 mins on ice between each round. 10 μL of sonicated sample was set aside as input control. Then 500 μL of dilution buffer (0.01% SDS, 1.1% Triton X-100, 1.2 mM EDTA, 16.7 mM Tris-HCl pH 8.1, and 167 mM NaCl) containing protease inhibitor cocktail (MilliporeSigma 05892791001) and 1 μL of anti-V5 (Thermo Fisher Scientific R960-25) was added to the sonicated sample. ChIP samples were rotated end over end overnight at 4° C.

For each ChIP, 50 L of Protein A/G Magnetic Beads (Thermo Fisher Scientific 88802) was washed with 1 mL of blocking buffer (0.5% TWEEN and 0.5% BSA in PBS) containing protease inhibitor cocktail (MilliporeSigma 05892791001) twice before resuspending in 100 μL of blocking buffer. ChIP samples were transferred to the beads and rotated end over end for 1 h at 4° C. ChIP supernatant was then removed and the beads were washed twice with 200 μL of RIPA low salt buffer (0.1% SDS, 1% Triton x-100, 1 mM EDTA, 20 mM Tris-HCl pH 8.1, 140 mM NaCl, 0.1% DOC), twice with 200 μL of RIPA high salt buffer (0.1% SDS, 1% Triton x-100, 1 mM EDTA, 20 mM Tris-HCl pH 8.1, 500 mM NaCl, 0.1% DOC), twice with 200 μL of LiCl wash buffer (250 mM LiCl, 1% NP40, 1% DOC, 1 mM EDTA, 10 mM Tris-HCl pH 8.1), and twice with 200 μL of TE (10 mM Tris-HCl pH8.0, 1 mM EDTA pH 8.0). ChIP samples were eluted with 50 μL of elution buffer (10 mM Tris-HCl pH 8.0, 5 mM EDTA, 300 mM NaCl, 0.1% SDS). 40 μL of water was added to the input control samples. 8 μL of reverse cross-linking buffer (250 mM Tris-HCl pH 6.5, 62.5 mM EDTA pH 8.0, 1.25 M NaCl, 5 mg/ml Proteinase K, 62.5 μg/ml RNAse A) was added to the ChIP and input control samples and then incubated at 65° C. for 5 h. After reverse crosslinking, samples were purified using 116 μL of SPRIselect Reagent (Beckman Coulter B23318).

ChIP samples were prepared for NGS with NEBNext Ultra II DNA Library Prep Kit for Illumina (NEB E7645S) and deep-sequenced on the Illumina NextSeq platform (>60 million reads per condition). Bowtie (Langmead et al., 2009) was used to align paired-end reads to the human hg38 UCSC genome with command line options q -X 300 --sam --chunkmbs 512″. Next, biological replicates were merged and Model-based Analysis of ChIP-seq (MACS) (Feng et al., 2012) was run with command line options “-g hs -B -S --mfold 6,30” to identify TF peaks. HOMER (Heinz et al., 2010) was used to discover motifs in the TF peak regions identified by MACS. The findMotifsGenome.pl program from HOMER was run with the command line options “-size 200 -mask” and the top 3 known and de novo motifs were presented. TFs were considered potential regulators of a candidate gene if the TF peak region identified by MACS overlapped with the 20 kb region centered around the transcriptional start site of the candidate gene based on RefSeq annotations.

Indel analysis. Cells plated in 96-well plates were grown to 60-80% confluency and assessed for indel rates as previously described (Joung et al., 2017b). Genomic DNA was harvested from cells using QuickExtract DNA Extraction kit (Lucigen QE09050). The genomic region flanking the site of interest was amplified using NEBNext High Fidelity 2x PCR Master Mix (New England BioLabs M0541L), first with region-specific primers (Table 13) for 15 cycles and then with barcoded primers for 15 cycles as previously described. PCR products were sequenced on the Illumina MiSeq platform (>10,000 reads per condition), and indel analysis was performed as previously described (Joung et al., 2017b).

Click-iT EdU flow cytometry assay. Cells plated in 24-well plates were differentiated and EdU incorporation was measured using the Click-iT EdU Alexa Fluor 488 Flow Cytometry Assay Kit (Thermo Fisher Scientific C10420) according to a modified version of the manufacturer's instructions. EdU was added to the culture medium to a final concentration of 10 μM for 2 h before cells were dissociated with Accutase (STEMCELL Technologies 07920) for 15-45 mins at 37° C. Cells were transferred to a 96-well plate, pelleted at 200×g for 5 mins, and washed once with 200 μL of 1% BSA (MilliporeSigma A9418) in PBS. Cells were resuspended in 100 μL of Click-iT fixative and incubated for 15 mins at room temperature in the dark. After fixing, cells were washed with 200 μL of 1% BSA (MilliporeSigma A9418) in PBS twice, resuspended in 100 μL of Click-iT saponin-based permeabilization and wash reagent, and incubated for 15 mins in the dark. To each sample, 500 μL of Click-iT reaction cocktail was added and the reaction mixture was incubated for 30 mins at room temperature in the dark. Cells were washed with 200 μL of Click-iT saponin-based permeabilization and wash reagent twice and resuspended in 200 μL of 1% BSA (MilliporeSigma A9418) in PBS before analysis on a CytoFLEX Flow Cytometer (Beckman Coulter). For each sample, 10,000 cells were analyzed with FlowJo (FlowJo). Significance testing was performed using Student's t-test.

Electrophysiology. Whole-cell patch-clamp recordings were performed as described (doi: 10.1016/j.celrep.2018.04.066). Recording pipettes were pulled from thin-walled borosilicate glass capillary tubing (KG33, King Precision Glass, CA, USA) on a P-97 puller (Sutter Instrument, CA, USA) and had resistances of 3-5 M2 when filled with internal solution (in mM: 128 K-gluconate, 10 HEPES, 10 phosphocreatine sodium salt, 1.1 EGTA, 5 ATP magnesium salt and 0.4 GTP sodium salt, pH=7.3, 300-305 mOsm). The cultured cells were constantly perfused at a speed of 3 ml/min with the extracellular solution (119 mM NaCl, 2.3 mM KCl, 2 mM CaCl2, 1 mM MgCl2, 15 mM HEPES, 5 mM glucose, pH=7.3-7.4, Osmolarity was adjusted to 325 mOsm with sucrose). All the experiments were performed at room temperature unless otherwise specified.

Cells were visualized with a 40X water-immersion objective on an upright microscope (Olympus, Japan) equipped with IR-DIC. Recordings were made using a Multiclamp 700B amplifier (Molecular Devices, CA, USA) and Clampex 10.7 software (Molecular Devices, CA, USA). In current clamp mode, membrane potential was held at −65 mV with a Multiclamp 700B amplifier, and step currents were then injected to elicit action potentials. Subsequent analysis was performed using Clampfit 10.7 software (Molecular Devices, CA, USA). The spontaneous AMPA receptor mediated excitatory postsynaptic currents (sEPSCs) were recorded after entering whole-cell path clamp recording mode at least for 3 min. The data were stored on a computer for subsequent off-line analysis. Cells in which the series resistance (Rs) changed by >20% were excluded for data analysis. In addition, cells with Rs more than 20 MΩ at any time during the recordings were discarded.

Reagent availability. The pooled and arrayed versions of MORF have been deposited at Addgene for distribution to the scientific community.

Code availability. Applicants have provided a Python script for aggregating gene lists from different datasets and selecting marker genes and TFs from MORF on the Feng Zhang lab GitHub page (github.com/fengzhanglab/TF_screen_manuscript).

### REFERENCES

1 Cohen, D. E. & Melton, D. Turning straw into gold: directing cell fate for regenerative medicine. Nat Rev Genet 12, 243-252, doi: 10.1038/nrg2938 (2011).
2 Colman, A. & Dreesen, O. Pluripotent stem cells and disease modeling. Cell Stem Cell 5, 244-247, doi: 10.1016/j.stem.2009.08.010 (2009).
3 Keller, G. Embryonic stem cell differentiation: emergence of a new era in biology and medicine. Genes Dev 19, 1129-1155, doi: 10.1101/gad.1303605 (2005).
4 Kiskinis, E. & Eggan, K. Progress toward the clinical application of patient-specific pluripotent stem cells. J Clin Invest 120, 51-59, doi: 10.1172/JCI40553 (2010).
5 Robinton, D. A. & Daley, G. Q. The promise of induced pluripotent stem cells in research and therapy. Nature 481, 295-305, doi: 10.1038/nature10761 (2012).
6 Furuyama, K. et al. Diabetes relief in mice by glucose-sensing insulin-secreting human alpha-cells. Nature, doi: 10.1038/s41586-019-0942-8 (2019).
7 Pang, Z. P. et al. Induction of human neuronal cells by defined transcription factors. Nature 476, 220-223, doi:10.1038/nature10202 (2011).
8 Song, K. et al. Heart repair by reprogramming non-myocytes with cardiac transcription factors. Nature 485, 599-604, doi: 10.1038/nature11139 (2012).
9 Sugimura, R. et al. Haematopoietic stem and progenitor cells from human pluripotent stem cells. Nature 545, 432-438, doi: 10.1038/nature22370 (2017).
10 Takahashi, K. et al. Induction of pluripotent stem cells from adult human fibroblasts by defined factors. Cell 131, 861-872, doi:10.1016/j.cell.2007.11.019 (2007).
11 Weintraub, H. et al. Activation of muscle-specific genes in pigment, nerve, fat, liver, and fibroblast cell lines by forced expression of MyoD. Proc Natl Acad Sci USA 86, 5434-5438 (1989).
12 Zhang, Y. et al. Rapid single-step induction of functional neurons from human pluripotent stem cells. Neuron 78, 785-798, doi:10.1016/j.neuron.2013.05.029 (2013).
13 Zhang, S. C., Wernig, M., Duncan, I. D., Brustle, O. & Thomson, J. A. In vitro differentiation of transplantable neural precursors from human embryonic stem cells. Nat Biotechnol 19, 1129-1133, doi: 10.1038/nbt1201-1129 (2001).
14 Chambers, S. M. et al. Highly efficient neural conversion of human ES and iPS cells by dual inhibition of SMAD signaling. Nat Biotechnol 27, 275-280, doi:10.1038/nbt.1529 (2009).
15 Hu, B. Y. et al. Neural differentiation of human induced pluripotent stem cells follows developmental principles but with variable potency. Proc Natl Acad Sci USA 107, 4335-4340, doi:10.1073/pnas.0910012107 (2010).
16 Konermann, S. et al. Genome-scale transcriptional activation by an engineered CRISPR-Cas9 complex. Nature 517, 583-588, doi: 10.1038/nature14136 (2015).
17 Camp, J. G. et al. Human cerebral organoids recapitulate gene expression programs of fetal neocortex development. Proc Natl Acad Sci USA 112, 15672-15677, doi:10.1073/pnas.1520760112 (2015).
18 Johnson, M. B. et al. Single-cell analysis reveals transcriptional heterogeneity of neural progenitors in human cortex. Nat Neurosci 18, 637-646, doi:10.1038/nn.3980 (2015).
19 Llorens-Bobadilla, E. et al. Single-Cell Transcriptomics Reveals a Population of Dormant Neural Stem Cells that Become Activated upon Brain Injury. Cell Stem Cell 17, 329-340, doi:10.1016/j.stem.2015.07.002 (2015).
20 Pollen, A. A. et al. Molecular identity of human outer radial glia during cortical development. Cell 163, 55-67, doi:10.1016/j.cell.2015.09.004 (2015).
21 Shin, J. et al. Single-Cell RNA-Seq with Waterfall Reveals Molecular Cascades underlying Adult Neurogenesis. Cell Stem Cell 17, 360-372, doi:10.1016/j.stem.2015.07.013 (2015).
22 Thomsen, E. R. et al. Fixed single-cell transcriptomic characterization of human radial glial diversity. Nat Methods 13, 87-93, doi:10.1038/nmeth.3629 (2016).
23 Wu, J. Q. et al. Dynamic transcriptomes during neural differentiation of human embryonic stem cells revealed by short, long, and paired-end sequencing. Proc Natl Acad Sci USA 107, 5254-5259, doi:10.1073/pnas.0914114107 (2010).
24 Zhang, Y. et al. Purification and Characterization of Progenitor and Mature Human Astrocytes Reveals Transcriptional and Functional Differences with Mouse. Neuron 89, 37-53, doi:10.1016/j.neuron.2015.11.013 (2016).
25 Quadrato, G. et al. Cell diversity and network dynamics in photosensitive human brain organoids. Nature 545, 48-53, doi: 10.1038/nature22047 (2017).
26 Nowakowski, T. J. et al. Spatiotemporal gene expression trajectories reveal developmental hierarchies of the human cortex. Science 358, 1318-1323, doi:10.1126/science.aap8809 (2017).
27 Casarosa, S., Fode, C. & Guillemot, F. Mash1 regulates neurogenesis in the ventral telencephalon. Development 126, 525-534 (1999).
28 Zhang, X. et al. Pax6 is a human neuroectoderm cell fate determinant. Cell Stem Cell 7, 90-100, doi:10.1016/j.stem.2010.04.017 (2010).
29 Murre, C. et al. Interactions between heterologous helix-loop-helix proteins generate complexes that bind specifically to a common DNA sequence. Cell 58, 537-544 (1989).
30 Morotomi-Yano, K. et al. Human regulatory factor X 4 (RFX4) is a testis-specific dimeric DNA-binding protein that cooperates with other human RFX members. J Biol Chem 277, 836-842, doi:10.1074/jbc.M108638200 (2002).
31 O'Roak, B. J. et al. Multiplex targeted sequencing identifies recurrently mutated genes in autism spectrum disorders. Science 338, 1619-1622, doi:10.1126/science.1227764 (2012).
32 Smith, D. J. et al. Functional screening of 2 Mb of human chromosome 21q22.2 in transgenic mice implicates minibrain in learning defects associated with Down syndrome. Nat Genet 16, 28-36, doi:10.1038/ng0597-28 (1997).
33 Fotaki, V. et al. Dyrk1A haploinsufficiency affects viability and causes developmental delay and abnormal brain morphology in mice. Mol Cell Biol 22, 6636-6647 (2002).
34 Hammerle, B. et al. Transient expression of Mnb/Dyrkla couples cell cycle exit and differentiation of neuronal precursors by inducing p27KIP1 expression and suppressing NOTCH signaling. Development 138, 2543-2554, doi:10.1242/dev.066167 (2011).
35 Park, J. et al. Dyrk1A phosphorylates p53 and inhibits proliferation of embryonic neuronal cells. J Biol Chem 285, 31895-31906, doi:10.1074/jbc.M110.147520 (2010).
36 Yabut, O., Domogauer, J. & D'Arcangelo, G. Dyrk1A overexpression inhibits proliferation and induces premature neuronal differentiation of neural progenitor cells. J Neurosci 30, 4004-4014, doi: 10.1523/JNEUROSCI.4711-09.2010 (2010).
37 Soppa, U. et al. The Down syndrome-related protein kinase DYRK1A phosphorylates p27(Kip1) and Cyclin D1 and induces cell cycle exit and neuronal differentiation. Cell Cycle 13, 2084-2100, doi: 10.4161/cc.29104 (2014).
38 Ashique, A. M. et al. The Rfx4 transcription factor modulates Shh signaling by regional control of ciliogenesis. Sci Signal 2, ra70, doi: 10.1126/scisignal.2000602 (2009).
39 Blackshear, P. J. et al. Graded phenotypic response to partial and complete deficiency of a brain-specific transcript variant of the winged helix transcription factor RFX4. Development 130, 4539-4552, doi: 10.1242/dev.00661 (2003).
40 Joung, J. et al. Genome-scale CRISPR-Cas9 knockout and transcriptional activation screening. Nat Protoc 12, 828-863, doi:10.1038/nprot.2017.016 (2017).
41 Fulco, C. P. et al. Activity-by-Contact model of enhancer specificity from thousands of CRISPR perturbations. bioRxiv, 529990, doi: 10.1101/529990 (2019).
42 Zheng, G. X. et al. Massively parallel digital transcriptional profiling of single cells. Nat Commun 8, 14049, doi: 10.1038/ncomms14049 (2017).
43 Satija, R., Farrell, J. A., Gennert, D., Schier, A. F. & Regev, A. Spatial reconstruction of single-cell gene expression data. Nat Biotechnol 33, 495-502, doi:10.1038/nbt.3192 (2015).
44 Langmead, B., Trapnell, C., Pop, M. & Salzberg, S. L. Ultrafast and memory-efficient alignment of short DNA sequences to the human genome. Genome Biol 10, R25, doi:10.1186/gb-2009-10-3-r25 (2009).
45 Li, B. & Dewey, C. N. RSEM: accurate transcript quantification from RNA-Seq data with or without a reference genome. BMC Bioinformatics 12, 323, doi: 10.1186/1471-2105-12-323 (2011).
46 Consortium, E. P. The ENCODE (ENCyclopedia Of DNA Elements) Project. Science 306, 636-640, doi: 10.1126/science.1105136 (2004).
47 Feng, J., Liu, T., Qin, B., Zhang, Y. & Liu, X. S. Identifying ChIP-seq enrichment using MACS. Nat Protoc 7, 1728-1740, doi:10.1038/nprot.2012.101 (2012).
48 Heinz, S. et al. Simple combinations of lineage-determining transcription factors prime cis-regulatory elements required for macrophage and B cell identities. Mol Cell 38, 576-589, doi:10.1016/j.molcel.2010.05.004 (2010).
49 Campisi, J. (2001). Cellular senescence as a tumor-suppressor mechanism. Trends Cell Biol 11, S27-31.
50 Cao, J., Spielmann, M., Qiu, X., Huang, X., Ibrahim, D. M., Hill, A. J., Zhang, F., Mundlos, S., Christiansen, L., Steemers, F. J., et al. (2019). The single-cell transcriptional landscape of mammalian organogenesis. Nature 566, 496-502.
51 Damell, J. E., Jr. (2002). Transcription factors as targets for cancer therapy. Nat Rev Cancer 2, 740-749.
52 De Rubeis, S., He, X., Goldberg, A. P., Poultney, C. S., Samocha, K., Cicek, A. E., Kou, Y., Liu, L., Fromer, M., Walker, S., et al. (2014). Synaptic, transcriptional and chromatin genes disrupted in autism. Nature 515, 209-215.
53 Englund, C., Fink, A., Lau, C., Pham, D., Daza, R. A., Bulfone, A., Kowalczyk, T., and Hevner, R. F. (2005). Pax6, Tbr2, and Tbr1 are expressed sequentially by radial glia, intermediate progenitor cells, and postmitotic neurons in developing neocortex. J Neurosci 25, 247-251.
54 Frantz, G. D., Weimann, J. M., Levin, M. E., and McConnell, S. K. (1994). Otx 1 and Otx2 define layers and regions in developing cerebral cortex and cerebellum. J Neurosci 14, 5725-5740.
55 Gilbert, L. A., Horlbeck, M. A., Adamson, B., Villalta, J. E., Chen, Y., Whitehead, E. H., Guimaraes, C., Panning, B., Ploegh, H. L., Bassik, M. C., et al. (2014). Genome-Scale CRISPR-Mediated Control of Gene Repression and Activation. Cell 159, 647-661.
56 Gotz, M., Stoykova, A., and Gruss, P. (1998). Pax6 controls radial glia differentiation in the cerebral cortex. Neuron 21, 1031-1044.
57 Liu, Y., Yu, C., Daley, T. P., Wang, F., Cao, W. S., Bhate, S., Lin, X., Still, C., 2nd, Liu, H., Zhao, D., et al. (2018). CRISPR Activation Screens Systematically Identify Factors that Drive Neuronal Fate and Reprogramming. Cell Stem Cell 23, 758-771 e758.
58 Matsunaga, E., Nambu, S., Oka, M., and Iriki, A. (2015). Complex and dynamic expression of cadherins in the embryonic marmoset cerebral cortex. Dev Growth Differ 57, 474-483.
59 Reinchisi, G., Ijichi, K., Glidden, N., Jakovcevski, I., and Zecevic, N. (2012). COUP-TFII expressing interneurons in human fetal forebrain. Cereb Cortex 22, 2820-2830. Schafer, S. T., Paquola, A. C. M., Stern, S., Gosselin, D., Ku, M., Pena, M., Kuret, T. J. M., 60 Liyanage, M., Mansour, A. A., Jaeger, B. N., et al. (2019). Pathological priming causes developmental gene network heterochronicity in autistic subject-derived neurons. Nat Neurosci 22, 243-255.
61 Shi, Y., Kirwan, P., and Livesey, F. J. (2012a). Directed differentiation of human pluripotent stem cells to cerebral cortex neurons and neural networks. Nat Protoc 7, 1836-1846.
62 Shi, Y., Kirwan, P., Smith, J., Robinson, H. P., and Livesey, F. J. (2012b). Human cerebral cortex development from pluripotent stem cells to functional excitatory synapses. Nat Neurosci 15, 477-486, S471.
63 Steele-Perkins, G., Plachez, C., Butz, K. G., Yang, G., Bachurski, C. J., Kinsman, S. L., Litwack, E. D., Richards, L. J., and Gronostajski, R. M. (2005). The transcription factor gene Nfib is essential for both lung maturation and brain development. Mol Cell Biol 25, 685-698.
64 UniProt, C. (2015). UniProt: a hub for protein information. Nucleic Acids Res 43, D204-212.
65 Wolf, F. A., Angerer, P., and Theis, F. J. (2018). SCANPY: large-scale single-cell gene expression data analysis. Genome Biol 19, 15.
66 Zhang, H. M., Chen, H., Liu, W., Liu, H., Gong, J., Wang, H., and Guo, A. Y. (2012). AnimalTFDB: a comprehensive animal transcription factor database. Nucleic Acids Res 40, D144-149.
67 Zhang, H. M., Liu, T., Liu, C. J., Song, S., Zhang, X., Liu, W., Jia, H., Xue, Y., and Guo, A. Y. (2015). AnimalTFDB 2.0: a resource for expression, prediction and functional study of animal transcription factors. Nucleic Acids Res 43, D76-81.

### Tables

TABLE 1
TF ORF isoforms and respective ranks in the screens. The TF ORF library
consisted of 90 TF isoforms of 70 TF genes that were synthesized with a 24bp barcode
(SEQ ID NO: 1-90). Ranks of TF ORF isoforms in each NP screening method are reported.
For the arrayed screen, the median values of the SLC1A3 and VIM mRNA expression fold
changes at day 7 were averaged, and the TFs were ranked based on the average. “NA” ranks
represent cases where the SLC1A3 or VIM mRNA expression levels could not be detected.
For the reporter cell line screen, the median values of the TF barcode enrichment in the
SLC1A3 and VIM screens were averaged to determine the TF rank. For the Flow-FISH
screens, the TF ranks were determined based on the average TF barcode enrichment. For the
single-cell RNA-seq screen, TFs were ranked based on average correlation with radial glia
from human fetal cortex and organoids provided in existing datasets. “NA” indicates
that the TF barcode was not detected in the single-cell RNA-seq data.

| | | | | | | Flow- | Flow- | | |
|---|---|---|---|---|---|---|---|---|---|
| | | | | | | FISH | FISH | | |
| | | Barcode | | | | Rank | Rank | scRNA- | Perturb- |
| Gene | | (SEQ ID | Average | Arrayed | Reporter | (2 | (10 | seq | seq |
| Name | RefSeq Isoform | NO) | Rank | Rank | Rank | genes) | genes) | Rank | Rank |
| ARX | NM_139058 | 1 | NA | NA | 88 | 39 | 83 | 37 | 78 |
| ASCL1 | NM_004316 | 2 | 25 | 2 | 62 | 22 | 27 | 12 | 1 |
| BCL11A | NM_022893 | 3 | NA | NA | 9 | 61 | 38 | 21 | 17 |
| BCL11A | NM_138559 | 4 | 47.2 | 17 | 53 | 58 | 63 | 45 | 43 |
| BRIP1 | NM_032043 | 5 | NA | NA | 33 | 12 | 14 | 23 | 5 |
| CDK1 | NM_001170407 | 6 | 53.8 | 27 | 41 | 63 | 71 | 67 | 42 |
| CDK1 | NM_001786 | 7 | 45.8 | 22 | 30 | 56 | 56 | 65 | 54 |
| CENPA | NM_001042426 | 8 | 57.4 | 19 | 48 | 66 | 74 | 80 | 32 |
| CHAF1A | NM_005483 | 9 | 48.4 | 57 | 87 | 33 | 30 | 35 | 9 |
| CXXC1 | NM_014593 | 10 | NA | NA | 14 | 53 | 48 | 58 | 69 |
| E2F1 | NM_005225 | 11 | NA | NA | 86 | 90 | 90 | 26 | 29 |
| E2F2 | NM_004091 | 12 | NA | NA | 83 | 84 | 87 | 17 | 22 |
| E2F7 | NM_203394 | 13 | NA | NA | 15 | 27 | 41 | 24 | 39 |
| E2F8 | NM_001256372 | 14 | NA | NA | 84 | 14 | 15 | 42 | 12 |
| EGR1 | NM_001964 | 15 | 68.6 | 60 | 65 | 80 | 69 | 69 | 74 |
| EMX2 | NM_001165924 | 16 | 67.8 | 44 | 59 | 81 | 81 | 74 | 62 |
| EMX2 | NM_004098 | 17 | NA | NA | 46 | 50 | 88 | 43 | 8 |
| ENO1 | NM_001201483 | 18 | 53.6 | 40 | 39 | 67 | 60 | 62 | 25 |
| ENO1 | NM_001428 | 19 | 61 | 33 | 64 | 71 | 77 | 60 | 59 |
| EOMES | NM_001278183 | 20 | 61.8 | 41 | 67 | 68 | 61 | 72 | 76 |
| EOMES | NM_005442 | 21 | 11.2 | 3 | 19 | 2 | 10 | 22 | 15 |
| FANCD2 | NM_001018115 | 22 | NA | NA | 57 | 10 | 17 | NA | NA |
| FEZF2 | NM_018008 | 23 | 54.4 | 12 | 79 | 88 | 84 | 9 | 30 |
| FOS | NM_005252 | 24 | 20.8 | 6 | 4 | 7 | 9 | 78 | 86 |
| FOXG1 | NM_005249 | 25 | 59.6 | 32 | 85 | 65 | 59 | 57 | 77 |
| FOXM1 | NM_001243089 | 26 | 73.8 | 54 | 71 | 85 | 89 | 70 | 23 |
| FOXM1 | NM_202003 | 27 | 71.6 | 51 | 72 | 87 | 85 | 63 | 18 |
| FOXN4 | NM_213596 | 28 | NA | NA | 28 | 41 | 46 | 36 | 45 |
| GLI3 | NM_000168 | 29 | 42.8 | 46 | 1 | 75 | 42 | 50 | 84 |
| H2AFX | NM_002105 | 30 | 62.4 | 26 | 61 | 72 | 76 | 77 | 67 |
| HELLS | NM_001289068 | 31 | 29.4 | 39 | 7 | 25 | 24 | 52 | 37 |
| HELLS | NM_001289073 | 32 | 36.8 | 36 | 32 | 40 | 36 | 40 | 24 |
| HES1 | NM_005524 | 33 | NA | NA | 24 | 6 | 7 | 20 | 63 |
| HES5 | NM_001010926 | 34 | 63.4 | 53 | 77 | 79 | 52 | 56 | 41 |
| HMGB1 | NM_002128 | 35 | 46 | 45 | 40 | 57 | 54 | 34 | 36 |
| HMGB2 | NM_002129 | 36 | 62 | 52 | 54 | 64 | 67 | 73 | 53 |
| HOPX | NM_139212 | 37 | 67 | 30 | 63 | 78 | 80 | 84 | 52 |
| ID3 | NM_002167 | 38 | 42.2 | 20 | 45 | 43 | 39 | 64 | 81 |
| ID4 | NM_001546 | 39 | 31.4 | 14 | 47 | 35 | 20 | 41 | 50 |
| INSM1 | NM_002196 | 40 | 50 | 35 | 13 | 86 | 78 | 38 | 82 |
| KLF15 | NM_014079 | 41 | NA | NA | 34 | 26 | 28 | 47 | 80 |
| LHX2 | NM_004789 | 42 | 12.6 | 13 | 25 | 9 | 6 | 10 | 55 |
| MAZ | NM_001276275 | 43 | NA | NA | 70 | 82 | 73 | 51 | 31 |
| MAZ | NM_001276276 | 44 | 69.4 | 29 | 66 | 83 | 82 | 87 | 65 |
| MEIS1 | NM_002398 | 45 | 26.4 | 23 | 26 | 34 | 34 | 15 | 19 |
| MXD3 | NM_001142935 | 46 | 52 | 59 | 44 | 48 | 55 | 54 | 75 |
| MXD3 | NM_031300 | 47 | 44.4 | 56 | 43 | 55 | 43 | 25 | 64 |
| MYBL2 | NM_001278610 | 48 | 68.6 | 49 | 82 | 70 | 66 | 76 | 79 |
| NFATC4 | NM_001198966 | 49 | 48.4 | 15 | 78 | 89 | 44 | 16 | 14 |
| NFATC4 | NM_001288802 | 50 | 66 | 55 | 90 | 51 | 75 | 59 | 83 |
| NFIB | NM_001190737 | 51 | 5 | 1 | 5 | 8 | 8 | 3 | 16 |
| NFIB | NM_005596 | 52 | 11.6 | 5 | 18 | 17 | 12 | 6 | 11 |
| NFIC | NM_001245004 | 53 | 11.4 | 10 | 35 | 3 | 5 | 4 | 3 |
| NFIC | NM_005597 | 54 | 7.6 | 7 | 21 | 4 | 4 | 2 | 6 |
| NOTCH1 | NM_017617 | 55 | NA | NA | 75 | 28 | 31 | NA | NA |
| NOTCH2 | NM_024408 | 56 | NA | NA | 6 | 37 | 1 | NA | NA |
| NR1D1 | NM_021724 | 57 | 61.2 | 61 | 23 | 76 | 64 | 82 | 66 |
| OTX1 | NM_014562 | 58 | 12.2 | 9 | 2 | 1 | 3 | 46 | 27 |
| PAX6 | NM_000280 | 59 | 13.2 | 8 | 20 | 15 | 18 | 5 | 38 |
| PAX6 | NM_001310159 | 60 | 13.6 | 18 | 8 | 16 | 19 | 7 | 26 |
| PAX6 | NM_001310160 | 61 | 65.8 | 48 | 56 | 69 | 70 | 86 | 56 |
| PLK4 | NM_001190799 | 62 | NA | NA | 42 | 31 | 29 | 27 | 85 |
| PLK4 | NM_001190801 | 63 | NA | NA | 74 | 18 | 33 | 14 | 2 |
| POU3F2 | NM_005604 | 64 | 54.8 | 58 | 55 | 62 | 86 | 13 | 13 |
| PRMT7 | NM_001184824 | 65 | 50.6 | 37 | 51 | 44 | 68 | 53 | 21 |
| RAD54L | NM_001142548 | 66 | 32 | 16 | 31 | 24 | 21 | 68 | 58 |
| RCOR2 | NM_173587 | 67 | 39.2 | 11 | 58 | 47 | 50 | 30 | 61 |
| RFX2 | NM_000635 | 68 | 39.8 | 31 | 50 | 45 | 45 | 28 | 70 |
| RFX2 | NM_134433 | 69 | NA | NA | 11 | 46 | 35 | 71 | 34 |
| RFX4 | NM_001206691 | 70 | 6.2 | 4 | 3 | 5 | 11 | 8 | 4 |
| RFX4 | NM_032491 | 71 | 42.6 | 21 | 22 | 59 | 62 | 49 | 40 |
| SMAD1 | NM_001003688 | 72 | 56.4 | 34 | 38 | 60 | 65 | 85 | 57 |
| SMARCC1 | NM_003074 | 73 | NA | NA | 76 | 20 | 22 | 39 | 73 |
| SOX1 | NM_005986 | 74 | NA | NA | 52 | 36 | 26 | 33 | 48 |
| SOX11 | NM_003108 | 75 | NA | NA | 89 | 54 | 51 | 32 | 68 |
| SOX2 | NM_003106 | 76 | NA | NA | 12 | 13 | 13 | 44 | 44 |
| SOX4 | NM_003107 | 77 | NA | NA | 81 | 21 | 32 | 19 | 87 |
| SOX9 | NM_000346 | 78 | NA | NA | 37 | 11 | 2 | 1 | 10 |
| SSRP1 | NM_003146 | 79 | 40.2 | 38 | 17 | 38 | 47 | 61 | 49 |
| STAT3 | NM_003150 | 80 | 43 | 24 | 16 | 42 | 58 | 75 | 33 |
| STAT3 | NM_213662 | 81 | 52.2 | 43 | 68 | 32 | 37 | 81 | 47 |
| TCF12 | NM_207037 | 82 | NA | NA | 10 | 23 | 16 | 31 | 20 |
| TCF12 | NM_207038 | 83 | NA | NA | 60 | 19 | 23 | 29 | 72 |
| TCF7L2 | NM_001146286 | 84 | NA | NA | 49 | 49 | 79 | 11 | 7 |
| TEAD2 | NM_001256662 | 85 | 69.6 | 47 | 73 | 77 | 72 | 79 | 46 |
| TEAD2 | NM_003598 | 86 | 45.8 | 25 | 69 | 29 | 40 | 66 | 51 |
| TRIM28 | NM_005762 | 87 | 53.6 | 50 | 36 | 74 | 53 | 55 | 35 |
| UHRF1 | NM_001290052 | 88 | 43.8 | 42 | 29 | 73 | 57 | 18 | 71 |
| YAP1 | NM_001195045 | 89 | 47.8 | 28 | 27 | 52 | 49 | 83 | 60 |
| ZFP36L1 | NM_001244701 | 90 | NA | NA | 80 | 30 | 25 | 48 | 28 |

| SEQ ID | |
|---|---|
| NO | Barcode |
| 1 | GACGATAGGTTGTGAGACGTAGAA |
| 2 | ATCCCTCTGGATTGCGCATAGGAC |
| 3 | TCGACGACGCTCAGCGGCCTATCA |
| 4 | GGTTGGATCTTGTAGGGAGCTCGT |
| 5 | CTTTCTTGCAGTCGCACCGAGCTC |
| 6 | GGTTAGAAATTCACGTTCCTATAC |
| 7 | GACTTGGGAACTCCGCAGAGACTG |
| 8 | GACTAGTATCCTTCATAGCTCCTC |
| 9 | ACTCCCTCCAAGGTGAGCGTCCTT |
| 10 | ACGCTGAAATGACAATTTCCGGAA |
| 11 | CGTATTTCGCACCCGGAAAGGGTG |
| 12 | TCGGCCCTGCCATCACTTCATAGT |
| 13 | TGCAAGAGCCACGGAGTTTATGTC |
| 14 | GCTAATAAGTGTCCGAGCACTCAC |
| 15 | CAGTGGAAACCTGACATTTACTTA |
| 16 | AGCGCTCGCACGCGCGTATCCATC |
| 17 | TTCCCTGCTGTCATGCTCACCATG |
| 18 | ACTCAGATACTGGGCCTTCCTTCT |
| 19 | GGTAATTATTGGGCGTTATCTCCA |
| 20 | GACTTGTTACATCGTTCCATAGGC |
| 21 | TACTGGAGCCGGCTACAGATGCTC |
| 22 | TTCCTTATACGAACGCCGATTGAT |
| 23 | GCCGCCACTTTCAGCCTCCGACGC |
| 24 | ATCACATTGAAGTCATTTACACCT |
| 25 | CGTGAACCTCAAGTCAAGCCAAAC |
| 26 | CTAGCCCGAAGGACACCTATTCCC |
| 27 | TGGGTTGTTCCGTTACTCATGAAG |
| 28 | TCCGAAAGCCTTGTTATTGAAATT |
| 29 | TAGCGGAAGTCTAACTGACTTCAT |
| 30 | CGTTTCTTATACCTCCGGCCATGG |
| 31 | GGCGCCGGGTTTGCAATTTCCCTG |
| 32 | CGCATATCGATCCTGATCCCGAGC |
| 33 | ATCTCCTGGGCCCGAAGTTGACGA |
| 34 | AGTGTCGAGCTATATGCAGCCGAA |
| 35 | TTCATGGTGCTCATGTCGTATTTC |
| 36 | AGGCCATTATTGGGTCCCGTGATT |
| 37 | TGCCTGAGTTAAAGACGCAGCTGC |
| 38 | AGAAGTCCGAACTATACTTGGATC |
| 39 | ATGGCTTCGTGACTGTGATTATCG |
| 40 | CCTTAATGATGCGAAGGGAAATAT |
| 41 | AGCACCAATGATCGCCGCTAGATG |
| 42 | ACCGTAGGAGCACGTTGCGATGAA |
| 43 | ACATGAACTATTAAACGAAACGCA |
| 44 | TCCAGGTCACCGGCGCGGCAGAAA |
| 45 | GTGGTGCTAACATTTAGTCGCTAG |
| 46 | GCTCACGCGGGATTAATGCGTCGT |
| 47 | TCGCACTTCTTATTCTGAGGCCCG |
| 48 | GCTCTTTCATTTCCTAGCTCCCGA |
| 49 | GTTATTTGGAAGACATTCACCTTG |
| 50 | TTCAATCCGTTCCCTCGCACAGTG |
| 51 | ACTGGTTCACGCAGAAACTGGTTC |
| 52 | AACGGCGAACGAGACAGACCAACG |
| 53 | ATAGGCAGAATCTACGAGAGGCTA |
| 54 | CTAGCCATCTTCCGCAATGGGACC |
| 55 | CGAATTATATTTGATTAACTGTCT |
| 56 | TCGAGCAGAAGGTGAGTGTCGGAC |
| 57 | ACGCTTGGACTGCACACGGAGCGT |
| 58 | GACGAATGAAACGCAGGACCGAGA |
| 59 | GATACGTGGTCACGTCGTCCCACA |
| 60 | GGACGCCAACACCTACTGTCATAG |
| 61 | GCTTCATAAACTAGTATCGGCAGA |
| 62 | GACCGTTGCTGGTTGTACATGCGC |
| 63 | CTGAGACACGTACTCCGGGTTCAC |
| 64 | AGTAACTTCAGTGATTTAACATTC |
| 65 | TCACAAACCGGAGACTGAGATCTT |
| 66 | CCATCAGATCCTACGCCCTCGCTC |
| 67 | AGCGCGGCGCACAACATGCTAACT |
| 68 | AGCATGACATAGTACCGGAAGCCA |
| 69 | ATTATAGCACATCCGCTACTTAGT |
| 70 | GAGTCCTCGTAATGCGACACAGTC |
| 71 | CTTATTATGCTATTCGCCTGAAAG |
| 72 | CGTAGGCAGTTAGGGCTGTCTAGA |
| 73 | AGAGTTGTTGAGCTGTCATGTGCA |
| 74 | GTGCGATACTACATGTACCGTATA |
| 75 | AGTAGTCATCTTATCTGTTCAGTC |
| 76 | ACAGAAGGTCCGCCATCGTGCATA |
| 77 | GGTCCGTTACAAATCCGATCTCCC |
| 78 | TGTGACGTTTAATCGTAGAAGACA |
| 79 | CTATCGAGTGTTGGTGATGACTTG |
| 80 | GATCTAGCGCGAGACTCCGAACAT |
| 81 | CGTGGGACCAGGGATATAGGGCCA |
| 82 | AACCTTAGGTAAAGGGCCTACAGC |
| 83 | TAAACAGGGAAAGTAATTCGGTGT |
| 84 | GTAAGGTAGTTGGATCCAAATGAG |
| 85 | AATCGAGGTAGCCAACGTATCGGG |
| 86 | AGGAGACCTCCACTGCCATGCATT |
| 87 | ACCTTGGCAGACCTATGGTGAGAG |
| 88 | ATTCGGTTTGGTGGATCTCCGGAC |
| 89 | ATTATCTCCGCGAGGACCGAATGG |
| 90 | CGAGGTTGTGACCTCCTGGGAACA |

TABLE 2
Radial glial cell markers

| Gene Name 1-20 | Gene Name 21-40 |
|---|---|
| VIM | FKBP10 |
| PTN | LFNG |
| SLC1A3 | FADS2 |
| CLU | EDNRB |
| CKB | PTPRZ1 |
| NR2E1 | FAM107A |
| NCAN | OAF |
| TTYH1 | TNC |
| MLC1 | SFXN5 |
| F3 | PDLIM4 |
| FABP7 | PDLIM3 |
| ATP1A2 | HES5 |
| DBI | SPARCL1 |
| GJA1 | COL11A1 |
| FJX1 | ATP1B2 |
| RGMA | DDAH1 |
| SLC1A2 | MT3 |
| PHGDH | ALDOC |
| GPR98 | NES |
| SCARA3 | PAX6 |

TABLE 3
TF isoforms in the barcoded human TF library. The TF library consisted of 1,836 genes covering 3,548 isoforms that overlapped between RefSeq
and Gencode annotations, as well as 2 control vectors expressing GFP and mCherry. 593 of the 3,548 isoforms were obtained from the Broad
Genomic Perturbation Platform (Broad GPP) and sequence verified. The rest of the isoforms were synthesized by Genewiz. Some of the Broad
GPP TF ORFs contained V5 epitope tags. Each TF has a unique 24-bp barcode that facilitates identification in pooled screens.

| | | | | | ORF se- | Bar- | |
|---|---|---|---|---|---|---|---|
| | | RefSeq | | Insert | quence | code | Epi- |
| | | Gene | | SEQ | SEQ | SEQ | tope |
| Source | Name | Name | RefSeq and Gencode ID | ID | ID | ID | Tag |
| Genewiz | TFORF0001 | HIF3A | NM_022462, ENST00000244303 | 91 | 3641 | 7191 | None |
| Genewiz | TFORF0002 | HIF3A | NM_152796, ENST00000472815 | 92 | 3642 | 7192 | None |
| Genewiz | TFORF0003 | HIF3A | NM_152794, ENST00000300862 | 93 | 3643 | 7193 | None |
| Genewiz | TFORF0004 | HIF3A | XM_005259153, ENST00000600383 | 94 | 3644 | 7194 | None |
| Genewiz | TFORF0005 | HIF3A | NM_152795, ENST00000377670 | 95 | 3645 | 7195 | None |
| Genewiz | TFORF0006 | TULP4 | XM_017011069, XM_017011070, NM_020245, ENST00000367097 | 96 | 3646 | 7196 | None |
| Genewiz | TFORF0007 | TULP4 | XM_017011071, NM_001007466, ENST00000367094 | 97 | 3647 | 7197 | None |
| Genewiz | TFORF0008 | ZNF709 | NM_152601, ENST00000397732 | 98 | 3648 | 7198 | None |
| Genewiz | TFORF0009 | ZNF708 | NM_021269, ENST00000356929 | 99 | 3649 | 7199 | None |
| Genewiz | TFORF0010 | ZNF879 | XM_011534550, XM_011534551, NM_001136116, | 100 | 3650 | 7200 | None |
| | | | XM_005265908, ENST00000444149 | | | | |
| Genewiz | TFORF0011 | ZNF878 | NM_001080404, ENST00000547628 | 101 | 3651 | 7201 | None |
| Genewiz | TFORF0012 | ZNF700 | NM_001271848, ENST00000622593 | 102 | 3652 | 7202 | None |
| Genewiz | TFORF0013 | ZNF700 | NM_144566, ENST00000254321 | 103 | 3653 | 7203 | None |
| Genewiz | TFORF0014 | ZNF707 | NM_001100599, NM_001100598, NM_173831, | 104 | 3654 | 7204 | None |
| | | | NM_001288806, NM_001288805, XM_011516977, | | | | |
| | | | ENST00000358656, ENST00000532158, ENST00000532205, | | | | |
| | | | ENST00000418203 | | | | |
| Genewiz | TFORF0015 | DRAP1 | NM_006442, ENST00000312515 | 105 | 3655 | 7205 | None |
| Genewiz | TFORF0016 | IRX5 | NM_005853, ENST00000394636 | 106 | 3656 | 7206 | None |
| Genewiz | TFORF0017 | IRX5 | NM_001252197, ENST00000320990 | 107 | 3657 | 7207 | None |
| Genewiz | TFORF0018 | IRX4 | NM_001278635, NM_001278633, ENST00000613726, | 108 | 3658 | 7208 | None |
| | | | ENST00000622814 | | | | |
| Genewiz | TFORF0019 | IRX4 | NM_016358, NM_001278634, NM_001278632, | 109 | 3659 | 7209 | None |
| | | | ENST00000513692, ENST00000231357, ENST00000505790 | | | | |
| Genewiz | TFORF0020 | IRX6 | NM_024335, ENST00000290552 | 110 | 3660 | 7210 | None |
| Genewiz | TFORF0021 | IRX1 | NM_024337, ENST00000302006 | 111 | 3661 | 7211 | None |
| Genewiz | TFORF0022 | IRX3 | NM_024336, ENST00000329734 | 112 | 3662 | 7212 | None |
| Genewiz | TFORF0023 | IRX2 | XM_011513979, NM_033267, NM_001134222, | 113 | 3663 | 7213 | None |
| | | | ENST00000382611, ENST00000302057 | | | | |
| Genewiz | TFORF0024 | FOXQ1 | NM_033260, ENST00000296839 | 114 | 3664 | 7214 | None |
| Genewiz | TFORF0025 | DHX9 | NM_001357, ENST00000367549 | 115 | 3665 | 7215 | None |
| Genewiz | TFORF0026 | ZNF45 | NM_003425, XM_017027224, XM_017027221, | 116 | 3666 | 7216 | None |
| | | | XM_017027217, XM_017027222, XM_011527267, | | | | |
| | | | XM_017027219, XM_017027218, XM_011527269, | | | | |
| | | | XM_011527273, XM_017027223, XM_017027220, | | | | |
| | | | XM_017027225, XM_017027226, XM_017027227, | | | | |
| | | | XM_011527271, ENST00000269973, ENST00000615985, | | | | |
| | | | ENST00000589703 | | | | |
| Genewiz | TFORF0027 | ZNF44 | NM_001164276, ENST00000356109 | 117 | 3667 | 7217 | None |
| Genewiz | TFORF0028 | ZNF44 | NM_016264, ENST00000355684 | 118 | 3668 | 7218 | None |
| Genewiz | TFORF0029 | ZNF43 | NM_001256653, ENST00000357491 | 119 | 3669 | 7219 | None |
| Genewiz | TFORF0030 | ZNF43 | NM_003423, ENST00000354959 | 120 | 3670 | 7220 | None |
| Genewiz | TFORF0031 | ZNF43 | XM_011528259, XM_011528257, XM_017027214, | 121 | 3671 | 7221 | None |
| | | | NM_001256648, NM_001256649, NM_001256650, | | | | |
| | | | XM_017027209, XM_017027210, XM_017027208, | | | | |
| | | | XM_017027216, XM_017027207, XM_017027212, | | | | |
| | | | XM_017027213, XM_017027211, XM_017027215, | | | | |
| | | | ENST00000594012, ENST00000595461, ENST00000598381 | | | | |
| Genewiz | TFORF0032 | GSC2 | NM_005315, ENST00000086933 | 122 | 3672 | 7222 | None |
| Genewiz | TFORF0033 | SP1 | NM_003109, XM_011538696, ENST00000426431 | 123 | 3673 | 7223 | None |
| Genewiz | TFORF0034 | SP2 | NM_003110, ENST00000376741 | 124 | 3674 | 7224 | None |
| Genewiz | TFORF0035 | SP3 | NM_003111, ENST00000310015 | 125 | 3675 | 7225 | None |
| Genewiz | TFORF0036 | SP3 | NM_001017371, ENST00000418194, ENST00000640958 | 126 | 3676 | 7226 | None |
| Genewiz | TFORF0037 | SP5 | NM_001003845, ENST00000375281 | 127 | 3677 | 7227 | None |
| Genewiz | TFORF0038 | SP7 | NM_001300837, XM_011537900, ENST00000537210 | 128 | 3678 | 7228 | None |
| Genewiz | TFORF0039 | SP7 | NM_152860, NM_001173467, ENST00000536324, | 129 | 3679 | 7229 | None |
| | | | ENST00000303846 | | | | |
| Genewiz | TFORF0040 | ZNF676 | NM_001001411, ENST00000397121 | 130 | 3680 | 7230 | None |
| Genewiz | TFORF0041 | ZNF675 | NM_138330, ENST00000359788 | 131 | 3681 | 7231 | None |
| Genewiz | TFORF0042 | ZNF674 | NM_001146291, ENST00000414387 | 132 | 3682 | 7232 | None |
| Genewiz | TFORF0043 | ZNF674 | NM_001039891, XM_011543941, ENST00000523374 | 133 | 3683 | 7233 | None |
| Genewiz | TFORF0044 | ZNF672 | NM_024836, XM_005270336, ENST00000306562 | 134 | 3684 | 7234 | None |
| Genewiz | TFORF0045 | ZNF671 | NM_024833, ENST00000317398 | 135 | 3685 | 7235 | None |
| Genewiz | TFORF0046 | MAX | NM_145113, ENST00000618858, ENST00000394606, | 136 | 3686 | 7236 | None |
| | | | ENST00000553928, ENST00000556979 | | | | |
| Genewiz | TFORF0047 | MAX | NM_001320415, XM_017021313, XM_017021312, | 137 | 3687 | 7237 | None |
| | | | ENST00000557277 | | | | |
| Genewiz | TFORF0048 | MAX | NM_197957, ENST00000341653 | 138 | 3688 | 7238 | None |
| Genewiz | TFORF0049 | MAX | NM_145112, ENST00000358402 | 139 | 3689 | 7239 | None |
| Genewiz | TFORF0050 | MAZ | NM_001276276, ENST00000562337 | 140 | 3690 | 7240 | None |
| Genewiz | TFORF0051 | MAZ | NM_002383, ENST00000322945 | 141 | 3691 | 7241 | None |
| Genewiz | TFORF0052 | MAZ | NM_001276275, ENST00000545521 | 142 | 3692 | 7242 | None |
| Genewiz | TFORF0053 | MAZ | NM_001042539, ENST00000219782 | 143 | 3693 | 7243 | None |
| Genewiz | TFORF0054 | ZNF679 | NM_153363, XM_017011797, ENST00000421025, | 144 | 3694 | 7244 | None |
| | | | ENST00000255746 | | | | |
| Genewiz | TFORF0055 | ZNF678 | NM_178549 | 145 | 3695 | 7245 | None |
| Genewiz | TFORF0056 | MAF | XM_017023234, XM_017023233, XM_017023235, | 146 | 3696 | 7246 | None |
| | | | ENST00000569649 | | | | |
| Genewiz | TFORF0057 | MAF | NM_001031804, ENST00000393350 | 147 | 3697 | 7247 | None |
| Genewiz | TFORF0058 | MAF | NM_005360, ENST00000326043 | 148 | 3698 | 7248 | None |
| Genewiz | TFORF0059 | CTBP1 | NM_001328, ENST00000290921 | 149 | 3699 | 7249 | None |
| Genewiz | TFORF0060 | CTBP2 | NM_001321014, NM_001290215, NM_001321013, | 150 | 3700 | 7250 | None |
| | | | NM_001321012, NM_001290214, NM_001329, | | | | |
| | | | NM_001083914, XM_017015757, XM_011539355, | | | | |
| | | | XM_005269567, XM_005269564, XM_005269571, | | | | |
| | | | XM_017015756, XM_011539354, XM_011539351, | | | | |
| | | | XM_006717642, XM_011539353, XM_005269561, | | | | |
| | | | XM_005269569, XM_005269568, XM_005269572, | | | | |
| | | | ENST00000337195, ENST00000531469, ENST00000494626, | | | | |
| | | | ENST00000411419 | | | | |
| Genewiz | TFORF0061 | CTBP2 | XM_011539349, ENST00000334808 | 151 | 3701 | 7251 | None |
| Genewiz | TFORF0062 | CTBP2 | NM_022802, ENST00000309035 | 152 | 3702 | 7252 | None |
| Genewiz | TFORF0063 | GTF3C2 | NM_001521, NM_001035521, ENST00000359541, | 153 | 3703 | 7253 | None |
| | | | ENST00000264720 | | | | |
| Genewiz | TFORF0064 | DENND4A | NM_005848, XM_017021863, XM_005254121, | 154 | 3704 | 7254 | None |
| | | | ENST00000431932 | | | | |
| Genewiz | TFORF0065 | DENND4A | NM_001144823, ENST00000443035 | 155 | 3705 | 7255 | None |
| Genewiz | TFORF0066 | ZNF451 | NM_001257273, ENST00000370708 | 156 | 3706 | 7256 | None |
| Genewiz | TFORF0067 | ZNF451 | NM_015555, ENST00000357489 | 157 | 3707 | 7257 | None |
| Genewiz | TFORF0068 | ZNF451 | NM_001031623, ENST00000370706 | 158 | 3708 | 7258 | None |
| Genewiz | TFORF0069 | CSDE1 | NM_007158, NM_001242893, ENST00000339438, | 159 | 3709 | 7259 | None |
| | | | ENST00000261443 | | | | |
| Genewiz | TFORF0070 | CSDE1 | NM_001242891, ENST00000610726, ENST00000438362 | 160 | 3710 | 7260 | None |
| Genewiz | TFORF0071 | CSDE1 | NM_001130523, ENST00000369530 | 161 | 3711 | 7261 | None |
| Genewiz | TFORF0072 | CSDE1 | NM_001007553, NM_001242892, ENST00000358528, | 162 | 3712 | 7262 | None |
| | | | ENST00000534699 | | | | |
| Genewiz | TFORF0073 | ZNF454 | NM_001178090, NM_182594, NM_001323306, | 163 | 3713 | 7263 | None |
| | | | NM_001178089, ENST00000320129, ENST00000519564 | | | | |
| Genewiz | TFORF0074 | BACH2 | NM_021813, NM_001170794, XM_017011166, | 164 | 3714 | 7264 | None |
| | | | XM_011536040, XM_017011165, XM_011536039, | | | | |
| | | | XM_005248759, ENST00000257749, ENST00000537989, | | | | |
| | | | ENST00000343122 | | | | |
| Genewiz | TFORF0075 | KCNIP4 | XM_017008653, NM_001035004, NM_147182, | 165 | 3715 | 7265 | None |
| | | | ENST00000359001, ENST00000509207 | | | | |
| Genewiz | TFORF0076 | KCNIP4 | NM_001035003, ENST00000382148 | 166 | 3716 | 7266 | None |
| Genewiz | TFORF0077 | KCNIP4 | NM_147183, ENST00000382150 | 167 | 3717 | 7267 | None |
| Genewiz | TFORF0078 | KCNIP4 | NM_147181, ENST00000447367 | 168 | 3718 | 7268 | None |
| Genewiz | TFORF0079 | KCNIP3 | NM_013434, ENST00000295225 | 169 | 3719 | 7269 | None |
| Genewiz | TFORF0080 | KCNIP3 | NM_001034914, ENST00000468529 | 170 | 3720 | 7270 | None |
| Genewiz | TFORF0081 | KCNIP2 | NM_014591, ENST00000461105 | 171 | 3721 | 7271 | None |
| Genewiz | TFORF0082 | KCNIP2 | NM_173194, ENST00000348850 | 172 | 3722 | 7272 | None |
| Genewiz | TFORF0083 | KCNIP2 | NM_173193, ENST00000353068 | 173 | 3723 | 7273 | None |
| Genewiz | TFORF0084 | KCNIP2 | NM_173195, ENST00000343195 | 174 | 3724 | 7274 | None |
| Genewiz | TFORF0085 | KCNIP2 | NM_173191, ENST00000356640 | 175 | 3725 | 7275 | None |
| Genewiz | TFORF0086 | LIN28B | XM_011535818, ENST00000637759 | 176 | 3726 | 7276 | None |
| Genewiz | TFORF0087 | LIN28A | NM_024674, XM_011542148, ENST00000326279, | 177 | 3727 | 7277 | None |
| | | | ENST00000254231 | | | | |
| Genewiz | TFORF0088 | ZNF408 | NM_024741, ENST00000311764 | 178 | 3728 | 7278 | None |
| Genewiz | TFORF0089 | ZEB1 | NM_001128128, ENST00000446923 | 179 | 3729 | 7279 | None |
| Genewiz | TFORF0090 | ZEB1 | NM_001174093, ENST00000560721 | 180 | 3730 | 7280 | None |
| Genewiz | TFORF0091 | ZEB1 | NM_001174096, ENST00000361642 | 181 | 3731 | 7281 | None |
| Genewiz | TFORF0092 | ZEB1 | NM_001174095, ENST00000542815 | 182 | 3732 | 7282 | None |
| Genewiz | TFORF0093 | ZEB1 | NM_030751, ENST00000320985 | 183 | 3733 | 7283 | None |
| Genewiz | TFORF0094 | ZEB2 | XM_017005414, XM_017005415, ENST00000638087, | 184 | 3734 | 7284 | None |
| | | | ENST00000638007, ENST00000636413, ENST00000637045, | | | | |
| | | | ENST00000637304 | | | | |
| Genewiz | TFORF0095 | ZEB2 | NM_001171653, ENST00000539609 | 185 | 3735 | 7285 | None |
| Genewiz | TFORF0096 | ZEB2 | NM_014795, XM_006712881, XM_006712882, | 186 | 3736 | 7286 | None |
| | | | ENST00000627532, ENST00000409487, ENST00000558170 | | | | |
| Genewiz | TFORF0097 | PSMD14 | NM_005805, ENST00000409682 | 187 | 3737 | 7287 | None |
| Genewiz | TFORF0098 | MSGN1 | NM_001105569, ENST00000281047 | 188 | 3738 | 7288 | None |
| Genewiz | TFORF0099 | HLF | XM_017024556, ENST00000430986, ENST00000573945, | 189 | 3739 | 7289 | None |
| | | | ENST00000575345 | | | | |
| Genewiz | TFORF0100 | NOV | NM_002514, ENST00000259526 | 190 | 3740 | 7290 | None |
| Genewiz | TFORF0101 | FOXC2 | NM_005251, ENST00000320354 | 191 | 3741 | 7291 | None |
| Genewiz | TFORF0102 | FOXC1 | NM_001453, ENST00000380874 | 192 | 3742 | 7292 | None |
| Genewiz | TFORF0103 | LEUTX | NM_001143832, ENST00000396841 | 193 | 3743 | 7293 | None |
| Genewiz | TFORF0104 | SMAD9 | NM_001127217, ENST00000399275, ENST00000379826 | 194 | 3744 | 7294 | None |
| Genewiz | TFORF0105 | TRERF1 | NM_001297573, XM_017011048, ENST00000541110 | 195 | 3745 | 7295 | None |
| Genewiz | TFORF0106 | TRERF1 | XM_017011053, XM_017011052, ENST00000340840 | 196 | 3746 | 7296 | None |
| Genewiz | TFORF0107 | TRERF1 | NM_033502, XM_017011049, ENST00000372922 | 197 | 3747 | 7297 | None |
| Genewiz | TFORF0108 | TRERF1 | XM_017011054, ENST00000354325 | 198 | 3748 | 7298 | None |
| Genewiz | TFORF0109 | SMAD6 | NM_005585, ENST00000288840 | 199 | 3749 | 7299 | None |
| Genewiz | TFORF0110 | SMAD7 | NM_001190821, ENST00000589634 | 200 | 3750 | 7300 | None |
| Genewiz | TFORF0111 | SMAD7 | NM_001190822, ENST00000591805 | 201 | 3751 | 7301 | None |
| Genewiz | TFORF0112 | SMAD2 | NM_001135937, XM_017025746, ENST00000356825, | 202 | 3752 | 7302 | None |
| | | | ENST00000586040 | | | | |
| Genewiz | TFORF0113 | SMAD3 | NM_001145104, ENST00000537194 | 203 | 3753 | 7303 | None |
| Genewiz | TFORF0114 | SMAD3 | NM_001145102, ENST00000540846 | 204 | 3754 | 7304 | None |
| Genewiz | TFORF0115 | SMAD3 | NM_001145103, ENST00000439724 | 205 | 3755 | 7305 | None |
| Genewiz | TFORF0116 | FOXM1 | NM_001243088, ENST00000627656 | 206 | 3756 | 7306 | None |
| Genewiz | TFORF0117 | FOXM1 | NM_202003, ENST00000361953 | 207 | 3757 | 7307 | None |
| Genewiz | TFORF0118 | FOXM1 | NM_202002, ENST00000342628 | 208 | 3758 | 7308 | None |
| Genewiz | TFORF0119 | ELMSAN1 | XM_005268204, XM_005268205, XM_005268206, | 209 | 3759 | 7309 | None |
| | | | ENST00000423556 | | | | |
| Genewiz | TFORF0120 | ELMSAN1 | NM_194278, NM_001043318, ENST00000394071, | 210 | 3760 | 7310 | None |
| | | | ENST00000286523 | | | | |
| Genewiz | TFORF0121 | BARX2 | NM_003658, ENST00000281437 | 211 | 3761 | 7311 | None |
| Genewiz | TFORF0122 | BARX1 | NM_021570, ENST00000253968 | 212 | 3762 | 7312 | None |
| Genewiz | TFORF0123 | OLIG2 | NM_005806, XM_005260908, ENST00000382357, | 213 | 3763 | 7313 | None |
| | | | ENST00000333337 | | | | |
| Genewiz | TFORF0124 | OLIG1 | NM_138983, ENST00000382348 | 214 | 3764 | 7314 | None |
| Genewiz | TFORF0125 | RLF | NM_012421, ENST00000372771 | 215 | 3765 | 7315 | None |
| Genewiz | TFORF0126 | CXXC1 | NM_014593, XM_017025718, ENST00000285106 | 216 | 3766 | 7316 | None |
| Genewiz | TFORF0127 | SP8 | NM_198956, ENST00000361443 | 217 | 3767 | 7317 | None |
| Genewiz | TFORF0128 | SP8 | NM_182700, ENST00000418710 | 218 | 3768 | 7318 | None |
| Genewiz | TFORF0129 | SP9 | NM_001145250, ENST00000394967 | 219 | 3769 | 7319 | None |
| Genewiz | TFORF0130 | HMGB3 | NM_001301229, NM_005342, NM_001301228, | 220 | 3770 | 7320 | None |
| | | | ENST00000325307, ENST00000448905 | | | | |
| Genewiz | TFORF0131 | HMGB4 | NM_145205, ENST00000519684, ENST00000522796 | 221 | 3771 | 7321 | None |
| Genewiz | TFORF0132 | FIZ1 | NM_032836, XM_005259352, ENST00000221665 | 222 | 3772 | 7322 | None |
| Genewiz | TFORF0133 | ZNF780A | NM_001142579, ENST00000414720 | 223 | 3773 | 7323 | None |
| Genewiz | TFORF0134 | ZNF780A | XM_011526772, XM_011526771, XM_005258773, | 224 | 3774 | 7324 | None |
| | | | XM_006723150, NM_001142577, ENST00000455521, | | | | |
| | | | ENST00000594395 | | | | |
| Genewiz | TFORF0135 | ZNF780A | XM_017026617, XM_017026618, XM_017026619, | 225 | 3775 | 7325 | None |
| | | | NM_001010880, NM_001142578, ENST00000340963, | | | | |
| | | | ENST00000595687 | | | | |
| Genewiz | TFORF0136 | ZNF780B | NM_001005851, XM_005258593, XM_017026427, | 226 | 3776 | 7326 | None |
| | | | XM_017026426, XM_017026425, ENST00000617676, | | | | |
| | | | ENST00000434248 | | | | |
| Genewiz | TFORF0137 | NFX1 | NM_002504, ENST00000379540 | 227 | 3777 | 7327 | None |
| Genewiz | TFORF0138 | NFX1 | NM_147134, ENST00000318524 | 228 | 3778 | 7328 | None |
| Genewiz | TFORF0139 | UBTF | NM_001076684, NM_001076683, XM_017025004, | 229 | 3779 | 7329 | None |
| | | | XM_017025003, ENST00000343638, ENST00000533177, | | | | |
| | | | ENST00000393606, ENST00000526094 | | | | |
| Genewiz | TFORF0140 | UBTF | NM_014233, XM_006722059, XM_006722061, | 230 | 3780 | 7330 | None |
| | | | XM_006722060, ENST00000302904, ENST00000436088, | | | | |
| | | | ENST00000529383 | | | | |
| Genewiz | TFORF0141 | POU5F1B | NM_001159542, ENST00000465342 | 231 | 3781 | 7331 | None |
| Genewiz | TFORF0142 | FXN | NM_001161706, ENST00000396364 | 232 | 3782 | 7332 | None |
| Genewiz | TFORF0143 | FXN | NM_181425, ENST00000396366 | 233 | 3783 | 7333 | None |
| Genewiz | TFORF0144 | PHOX2A | NM_005169, ENST00000298231 | 234 | 3784 | 7334 | None |
| Genewiz | TFORF0145 | PHOX2B | NM_003924, ENST00000226382 | 235 | 3785 | 7335 | None |
| Genewiz | TFORF0146 | THAP1 | NM_199003, ENST00000345117 | 236 | 3786 | 7336 | None |
| Genewiz | TFORF0147 | SNW1 | NM_001318844, ENST00000555761 | 237 | 3787 | 7337 | None |
| Genewiz | TFORF0148 | THAP4 | NM_001164356, ENST00000402136 | 238 | 3788 | 7338 | None |
| Genewiz | TFORF0149 | THAP4 | NM_015963, ENST00000407315 | 239 | 3789 | 7339 | None |
| Genewiz | TFORF0150 | THAPS | NM_182529, ENST00000313516 | 240 | 3790 | 7340 | None |
| Genewiz | TFORF0151 | THAP5 | NM_001130475, ENST00000415914 | 241 | 3791 | 7341 | None |
| Genewiz | TFORF0152 | THAP6 | NM_001317791, XM_017007800, ENST00000380837 | 242 | 3792 | 7342 | None |
| Genewiz | TFORF0153 | THAP6 | XM_005262774, ENST00000507556 | 243 | 3793 | 7343 | None |
| Genewiz | TFORF0154 | THAP7 | NM_030573, NM_001008695, ENST00000215742, | 244 | 3794 | 7344 | None |
| | | | ENST00000399133 | | | | |
| Genewiz | TFORF0155 | THAP9 | NM_024672, ENST00000302236 | 245 | 3795 | 7345 | None |
| Genewiz | TFORF0156 | EWSR1 | NM_013986, ENST00000414183 | 246 | 3796 | 7346 | None |
| Genewiz | TFORF0157 | EWSR1 | NM_001163286, ENST00000332035 | 247 | 3797 | 7347 | None |
| Genewiz | TFORF0158 | EWSR1 | NM_005243, ENST00000397938 | 248 | 3798 | 7348 | None |
| Genewiz | TFORF0159 | EWSR1 | NM_001163287, ENST00000333395 | 249 | 3799 | 7349 | None |
| Genewiz | TFORF0160 | EWSR1 | NM_001163285, ENST00000406548 | 250 | 3800 | 7350 | None |
| Genewiz | TFORF0161 | MLLT1 | NM_005934, ENST00000252674 | 251 | 3801 | 7351 | None |
| Genewiz | TFORF0162 | PES1 | NM_001282328, NM_001282327, XM_017028678, | 252 | 3802 | 7352 | None |
| | | | ENST00000402281, ENST00000405677 | | | | |
| Genewiz | TFORF0163 | PES1 | NM_001243225, ENST00000335214 | 253 | 3803 | 7353 | None |
| Genewiz | TFORF0164 | SIX4 | NM_017420, ENST00000216513 | 254 | 3804 | 7354 | None |
| Genewiz | TFORF0165 | SIX5 | NM_175875, ENST00000317578 | 255 | 3805 | 7355 | None |
| Genewiz | TFORF0166 | SIX6 | NM_007374, ENST00000327720 | 256 | 3806 | 7356 | None |
| Genewiz | TFORF0167 | SIX1 | NM_005982, ENST00000247182 | 257 | 3807 | 7357 | None |
| Genewiz | TFORF0168 | SIX2 | NM_016932, ENST00000303077 | 258 | 3808 | 7358 | None |
| Genewiz | TFORF0169 | SIX3 | NM_005413, ENST00000260653 | 259 | 3809 | 7359 | None |
| Genewiz | TFORF0170 | ZNF587B | NM_001204818, ENST00000442832 | 260 | 3810 | 7360 | None |
| Genewiz | TFORF0171 | ZFP69 | XM_006710606, NM_198494, NM_001320179, | 261 | 3811 | 7361 | None |
| | | | ENST00000372706, ENST00000372705 | | | | |
| Genewiz | TFORF0172 | ONECUT2 | NM_004852, ENST00000491143 | 262 | 3812 | 7362 | None |
| Genewiz | TFORF0173 | RELB | XM_005259127, ENST00000505236 | 263 | 3813 | 7363 | None |
| Genewiz | TFORF0174 | RELA | NM_021975, ENST00000406246 | 264 | 3814 | 7364 | None |
| Genewiz | TFORF0175 | RELA | NM_001243985, ENST00000612991 | 265 | 3815 | 7365 | None |
| Genewiz | TFORF0176 | RELA | NM_001145138, ENST00000308639 | 266 | 3816 | 7366 | None |
| Genewiz | TFORF0177 | HDGF | NM_001126051, ENST00000368209 | 267 | 3817 | 7367 | None |
| Genewiz | TFORF0178 | HDGF | NM_001126050, ENST00000368206 | 268 | 3818 | 7368 | None |
| Genewiz | TFORF0179 | ZFP62 | NM_152283, XM_017009714, XM_017009712, | 269 | 3819 | 7369 | None |
| | | | XM_017009710, XM_017009715, XM_017009713, | | | | |
| | | | XM_017009711, ENST00000512132 | | | | |
| Genewiz | TFORF0180 | ZFP62 | NM_001172638, XM_017009717, ENST00000502412 | 270 | 3820 | 7370 | None |
| Genewiz | TFORF0181 | ESX1 | NM_153448, ENST00000372588 | 271 | 3821 | 7371 | None |
| Genewiz | TFORF0182 | SHOX2 | NM_006884, ENST00000441443 | 272 | 3822 | 7372 | None |
| Genewiz | TFORF0183 | SHOX2 | NM_001163678, ENST00000483851 | 273 | 3823 | 7373 | None |
| Genewiz | TFORF0184 | SHOX2 | XM_006713728, XM_017007055, ENST00000425436 | 274 | 3824 | 7374 | None |
| Genewiz | TFORF0185 | SHOX2 | NM_003030, ENST00000389589 | 275 | 3825 | 7375 | None |
| Genewiz | TFORF0186 | DMRTC1 | XM_017029725, XM_005262288, XM_005262287, | 276 | 3826 | 7376 | None |
| | | | NM_033053, ENST00000595412 | | | | |
| Genewiz | TFORF0187 | DMRTC2 | XM_017027128, XM_017027126, XM_017027125, | 277 | 3827 | 7377 | None |
| | | | XM_017027127, ENST00000596827 | | | | |
| Genewiz | TFORF0188 | DMRTC2 | NM_001040283, ENST00000269945 | 278 | 3828 | 7378 | None |
| Genewiz | TFORF0189 | RFX8 | NM_001145664, ENST00000428343 | 279 | 3829 | 7379 | None |
| Genewiz | TFORF0190 | RFX4 | NM_213594, ENST00000392842 | 280 | 3830 | 7380 | None |
| Genewiz | TFORF0191 | RFX4 | NM_032491, ENST00000229387 | 281 | 3831 | 7381 | None |
| Genewiz | TFORF0192 | RFX4 | NM_001206691, ENST00000357881 | 282 | 3832 | 7382 | None |
| Genewiz | TFORF0193 | RFX5 | NM_000449, NM_001025603, XM_011509850, | 283 | 3833 | 7383 | None |
| | | | XM_011509848, XM_005245405, XM_011509849, | | | | |
| | | | XM_005245406, XM_011509847, ENST0290524, | | | | |
| | | | ENST00000368870, ENST00000452671 | | | | |
| Genewiz | TFORF0194 | RFX6 | NM_173560, ENST00000332958 | 284 | 3834 | 7384 | None |
| Genewiz | TFORF0195 | RFX7 | XM_017022508, XM_011521925, XM_017022507, | 285 | 3835 | 7385 | None |
| | | | ENST00000559447 | | | | |
| Genewiz | TFORF0196 | ZNF844 | NM_001136501, ENST00000439326 | 286 | 3836 | 7386 | None |
| Genewiz | TFORF0197 | RFX1 | NM_002918, ENST00000254325 | 287 | 3837 | 7387 | None |
| Genewiz | TFORF0198 | RFX2 | NM_134433, ENST00000592546 | 288 | 3838 | 7388 | None |
| Genewiz | TFORF0199 | RFX2 | NM_000635, XM_011528171, ENST00000303657, | 289 | 3839 | 7389 | None |
| | | | ENST00000359161 | | | | |
| Genewiz | TFORF0200 | RFX3 | NM_002919, ENST00000358730 | 290 | 3840 | 7390 | None |
| Genewiz | TFORF0201 | RFX3 | NM_001282117, ENST00000302303 | 291 | 3841 | 7391 | None |
| Genewiz | TFORF0202 | RFX3 | NM_134428, NM_001282116, ENST00000382004, | 292 | 3842 | 7392 | None |
| | | | ENST00000617270 | | | | |
| Genewiz | TFORF0203 | ELK1 | NM_001257168, ENST00000343894 | 293 | 3843 | 7393 | None |
| Genewiz | TFORF0204 | ELK4 | NM_001973, XM_005244950, XM_005244951, | 294 | 3844 | 7394 | None |
| | | | ENST00000616704, ENST00000357992 | | | | |
| Genewiz | TFORF0205 | DBP | NM_001352, ENST00000222122 | 295 | 3845 | 7395 | None |
| Genewiz | TFORF0206 | ETV3L | NM_001004341, ENST00000454449 | 296 | 3846 | 7396 | None |
| Genewiz | TFORF0207 | FOXP2 | NM_148899, ENST00000360232 | 297 | 3847 | 7397 | None |
| Genewiz | TFORF0208 | FOXP2 | NM_014491, ENST00000393494, ENST00000350908 | 298 | 3848 | 7398 | None |
| Genewiz | TFORF0209 | FOXP2 | XM_017012801, NM_148898, ENST00000408937 | 299 | 3849 | 7399 | None |
| Genewiz | TFORF0210 | FOXP2 | NM_148900, ENST00000403559 | 300 | 3850 | 7400 | None |
| Genewiz | TFORF0211 | FOXP3 | XM_006724533, ENST00000455775 | 301 | 3851 | 7401 | None |
| Genewiz | TFORF0212 | FOXP3 | NM_001114377, ENST00000376199, ENST00000518685 | 302 | 3852 | 7402 | None |
| Genewiz | TFORF0213 | FOXP1 | NM_001244813, XM_011533588, ENST00000614176 | 303 | 3853 | 7403 | None |
| Genewiz | TFORF0214 | FOXP1 | NM_001244816, NM_032682, XM_011533585, | 304 | 3854 | 7404 | None |
| | | | XM_006713102, XM_017006165, XM_011533584, | | | | |
| | | | XM_006713103, XM_006713104, XM_017006166, | | | | |
| | | | NM_001244814, ENST00000318789, ENST00000475937, | | | | |
| | | | ENST00000498215 | | | | |
| Genewiz | TFORF0215 | FOXP1 | NM_001244808, ENST00000493089 | 305 | 3855 | 7405 | None |
| Genewiz | TFORF0216 | FOXP1 | NM_001244812, ENST00000484350 | 306 | 3856 | 7406 | None |
| Genewiz | TFORF0217 | FOXP1 | NM_001244815, ENST00000491238 | 307 | 3857 | 7407 | None |
| Genewiz | TFORF0218 | FOXP1 | NM_001244810, ENST00000615603 | 308 | 3858 | 7408 | None |
| Genewiz | TFORF0219 | ADNP | NM_001282532, NM_001282531, NM_015339, | 309 | 3859 | 7409 | None |
| | | | NM_181442, XM_017027758, XM_017027759, | | | | |
| | | | XM_011528747, XM_017027757, ENST00000371602, | | | | |
| | | | ENST00000621696, ENST00000349014, ENST00000396029, | | | | |
| | | | ENST00000396032 | | | | |
| Genewiz | TFORF0220 | FOXP4 | NM_138457, ENST00000373063 | 310 | 3860 | 7410 | None |
| Genewiz | TFORF0221 | FOXP4 | NM_001012427, ENST00000373057 | 311 | 3861 | 7411 | None |
| Genewiz | TFORF0222 | FOXP4 | NM_001012426, ENST00000373060, ENST00000307972 | 312 | 3862 | 7412 | None |
| Genewiz | TFORF0223 | ZNF479 | XM_017012777, XM_011515604, XM_011515608, | 313 | 3863 | 7413 | None |
| | | | NM_033273, ENST00000331162, ENST00000319636 | | | | |
| Genewiz | TFORF0224 | ZNF592 | XM_005254996, XM_017022734, NM_014630, | 314 | 3864 | 7414 | None |
| | | | XM_011522247, XM_011522246, ENST00000560079, | | | | |
| | | | ENST00000299927 | | | | |
| Genewiz | TFORF0225 | ZNF593 | XM_017001398, ENST00000270812 | 315 | 3865 | 7415 | None |
| Genewiz | TFORF0226 | ZNF593 | NM_015871, ENST00000374266 | 316 | 3866 | 7416 | None |
| Genewiz | TFORF0227 | ZNF596 | NM_001287256, NM_001287255, NM_173539, | 317 | 3867 | 7417 | None |
| | | | NM_001042416, NM_001042415, NM_001287254, | | | | |
| | | | XM_017013166, ENST00000320552, ENST00000308811, | | | | |
| | | | ENST00000398612 | | | | |
| Genewiz | TFORF0228 | ZNF594 | NM_032530, XM_005256827, ENST00000399604, | 318 | 3868 | 7418 | None |
| | | | ENST00000575779 | | | | |
| Genewiz | TFORF0229 | ZNF595 | NM_001286052, ENST00000609518 | 319 | 3869 | 7419 | None |
| Genewiz | TFORF0230 | ZNF595 | NM_001286053, NM_001286054, ENST00000608255 | 320 | 3870 | 7420 | None |
| Genewiz | TFORF0231 | ZNF595 | NM_182524, ENST00000610261 | 321 | 3871 | 7421 | None |
| Genewiz | TFORF0232 | ZNF599 | NM_001007248, ENST00000329285 | 322 | 3872 | 7422 | None |
| Genewiz | TFORF0233 | MNX1 | NM_005515, ENST00000252971 | 323 | 3873 | 7423 | None |
| Genewiz | TFORF0234 | MNX1 | NM_001165255, ENST00000543409 | 324 | 3874 | 7424 | None |
| Genewiz | TFORF0235 | MYCN | NM_005378, NM_001293228, XM_017004168, | 325 | 3875 | 7425 | None |
| | | | ENST00000281043 | | | | |
| Genewiz | TFORF0236 | MYCN | NM_001293231, ENST00000638417 | 326 | 3876 | 7426 | None |
| Genewiz | TFORF0237 | ZNF600 | NM_198457, ENST00000338230 | 327 | 3877 | 7427 | None |
| Genewiz | TFORF0238 | MYCL | NM_001033082, ENST00000397332 | 328 | 3878 | 7428 | None |
| Genewiz | TFORF0239 | MYCL | NM_001033081, ENST00000372816 | 329 | 3879 | 7429 | None |
| Genewiz | TFORF0240 | MYCL | NM_005376, ENST00000372815 | 330 | 3880 | 7430 | None |
| Genewiz | TFORF0241 | ZNF606 | NM_025027, XM_005259276, ENST00000341164 | 331 | 3881 | 7431 | None |
| Genewiz | TFORF0242 | ZNF607 | NM_032689, XM_006723435, ENST00000355202 | 332 | 3882 | 7432 | None |
| Genewiz | TFORF0243 | ZNF607 | NM_001172677, XM_006723436, ENST00000395835 | 333 | 3883 | 7433 | None |
| Genewiz | TFORF0244 | SUB1 | XM_017008986, XM_017008987, NM_006713, | 334 | 3884 | 7434 | None |
| | | | XM_011513944, ENST00000506237, ENST00000512913, | | | | |
| | | | ENST00000265073, ENST00000515355, ENST00000502897 | | | | |
| Genewiz | TFORF0245 | CAMTAZ | NM_001171166, ENST00000381311 | 335 | 3885 | 7435 | None |
| Genewiz | TFORF0246 | CAMTA2 | NM_001171167, ENST00000414043 | 336 | 3886 | 7436 | None |
| Genewiz | TFORF0247 | CAMTA2 | XM_006721478, ENST00000572543 | 337 | 3887 | 7437 | None |
| Genewiz | TFORF0248 | CAMTA2 | NM_015099, ENST00000348066 | 338 | 3888 | 7438 | None |
| Genewiz | TFORF0249 | CAMTAZ | NM_001171168, ENST00000361571 | 339 | 3889 | 7439 | None |
| Genewiz | TFORF0250 | VEZF1 | NM_007146, ENST00000581208 | 340 | 3890 | 7440 | None |
| Genewiz | TFORF0251 | VEZF1 | XM_017025018, XM_017025017, ENST00000584396 | 341 | 3891 | 7441 | None |
| Genewiz | TFORF0252 | CAMTA1 | NM_001242701, ENST00000557126 | 342 | 3892 | 7442 | None |
| Genewiz | TFORF0253 | CAMTA1 | NM_015215, ENST00000303635 | 343 | 3893 | 7443 | None |
| Genewiz | TFORF0254 | CAMTA1 | NM_001195563, ENST00000473578 | 344 | 3894 | 7444 | None |
| Genewiz | TFORF0255 | TCF4 | NM_001243228, ENST00000564403 | 345 | 3895 | 7445 | None |
| Genewiz | TFORF0256 | TCF4 | NM_001243233, XM_017025956, ENST00000537856, | 346 | 3896 | 7446 | None |
| | | | ENST00000561992, ENST00000570177 | | | | |
| Genewiz | TFORF0257 | TCF4 | NM_001306208, ENST00000564228 | 347 | 3897 | 7447 | None |
| Genewiz | TFORF0258 | TCF4 | XM_017025949, XM_017025937, XM_017025948, | 348 | 3898 | 7448 | None |
| | | | ENST00000568740 | | | | |
| Genewiz | TFORF0259 | TCF4 | NM_001243236, ENST00000561831 | 349 | 3899 | 7449 | None |
| Genewiz | TFORF0260 | TCF4 | NM_001243226, ENST00000398339 | 350 | 3900 | 7450 | None |
| Genewiz | TFORF0261 | TCF4 | NM_001243234, ENST00000457482 | 351 | 3901 | 7451 | None |
| Genewiz | TFORF0262 | TCF4 | NM_001306207, XM_017025947, XM_017025936, | 352 | 3902 | 7452 | None |
| | | | XM_017025946, ENST00000540999 | | | | |
| Genewiz | TFORF0263 | TCF4 | XM_011526160, XM_005266754, XM_005266755, | 353 | 3903 | 7453 | None |
| | | | XM_005266752, ENST00000636822 | | | | |
| Genewiz | TFORF0264 | TCF4 | NM_001243227, XM_005266744, XM_005266743, | 354 | 3904 | 7454 | None |
| | | | XM_005266739, ENST00000636400, ENST00000568673, | | | | |
| | | | ENST00000537578 | | | | |
| Genewiz | TFORF0265 | TCF4 | NM_001243232, ENST00000544241 | 355 | 3905 | 7455 | None |
| Genewiz | TFORF0266 | TCF4 | NM_001243231, ENST00000543082 | 356 | 3906 | 7456 | None |
| Genewiz | TFORF0267 | TCF4 | XM_017025939, ENST00000638154 | 357 | 3907 | 7457 | None |
| Genewiz | TFORF0268 | TCF4 | NM_001243235, ENST00000570287 | 358 | 3908 | 7458 | None |
| Genewiz | TFORF0269 | TCF4 | NM_001243230, ENST00000566286 | 359 | 3909 | 7459 | None |
| Genewiz | TFORF0270 | TCF7 | NM_201632, NM_213648, ENST00000520958, | 360 | 3910 | 7460 | None |
| | | | ENST00000395023 | | | | |
| Genewiz | TFORF0271 | TCF7 | NM_001134851, ENST00000518915 | 361 | 3911 | 7461 | None |
| Genewiz | TFORF0272 | TCF7 | NM_201634, ENST00000378560 | 362 | 3912 | 7462 | None |
| Genewiz | TFORF0273 | TCF3 | XM_011528221, XM_006722857, XM_006722858, | 363 | 3913 | 7463 | None |
| | | | ENST00000453954 | | | | |
| Genewiz | TFORF0274 | TCF3 | XM_017027180, ENST00000395423 | 364 | 3914 | 7464 | None |
| Genewiz | TFORF0275 | TCF3 | NM_003200, XM_017027181, ENST00000262965, | 365 | 3915 | 7465 | None |
| | | | ENST00000611869 | | | | |
| Genewiz | TFORF0276 | TCF3 | NM_001136139, XM_017027182, ENST00000588136 | 366 | 3916 | 7466 | None |
| Genewiz | TFORF0277 | TCF3 | XM_011528227, ENST00000344749 | 367 | 3917 | 7467 | None |
| Genewiz | TFORF0278 | ARID2 | NM_152641, ENST00000334344 | 368 | 3918 | 7468 | None |
| Genewiz | TFORF0279 | ZNF776 | NM_173632, ENST00000317178 | 369 | 3919 | 7469 | None |
| Genewiz | TFORF0280 | IGFBP1 | NM_000596, ENST00000275525 | 370 | 3920 | 7470 | None |
| Genewiz | TFORF0281 | ZNF90 | NM_007138, ENST00000418063 | 371 | 3921 | 7471 | None |
| Genewiz | TFORF0282 | MEF2C | NM_002397, NM_001193350, XM_005248511, | 372 | 3922 | 7472 | None |
| | | | XM_011543396, XM_006714619, ENST00000504921, | | | | |
| | | | ENST00000437473, ENST00000636294 | | | | |
| Genewiz | TFORF0283 | MEF2C | XM_011543401, XM_017009482, ENST00000510942 | 373 | 3923 | 7473 | None |
| Genewiz | TFORF0284 | MEF2C | XM_017009483, ENST00000627659 | 374 | 3924 | 7474 | None |
| Genewiz | TFORF0285 | MEF2C | NM_001131005, XM_017009478, ENST00000424173, | 375 | 3925 | 7475 | None |
| | | | ENST00000625674 | | | | |
| Genewiz | TFORF0286 | MEF2C | NM_001193348, ENST00000628656 | 376 | 3926 | 7476 | None |
| Genewiz | TFORF0287 | MEF2C | XM_011543397, XM_017009475, ENST00000625585 | 377 | 3927 | 7477 | None |
| Genewiz | TFORF0288 | MEF2C | NM_001308002, XM_017009477, XM_017009476, | 378 | 3928 | 7478 | None |
| | | | ENST00000629612, ENST00000508569 | | | | |
| Genewiz | TFORF0289 | MEF2C | NM_001193349, ENST00000626391 | 379 | 3929 | 7479 | None |
| Genewiz | TFORF0290 | MEF2C | XM_011543400, XM_017009479, XM_017009481, | 380 | 3930 | 7480 | None |
| | | | XM_017009480, ENST00000636998, ENST00000514028, | | | | |
| | | | ENST00000637732, ENST00000514015 | | | | |
| Genewiz | TFORF0291 | MEF2C | NM_001193347, XM_006714625, ENST00000340208 | 381 | 3931 | 7481 | None |
| Genewiz | TFORF0292 | MEF2B | NM_001145785, ENST00000424583 | 382 | 3932 | 7482 | None |
| Genewiz | TFORF0293 | MEF2A | XM_011521586, NM_001130927, ENST00000558812 | 383 | 3933 | 7483 | None |
| Genewiz | TFORF0294 | MEF2A | XM_005254915, NM_001130926, NM_001171894, | 384 | 3934 | 7484 | None |
| | | | ENST00000338042, ENST00000557785 | | | | |
| Genewiz | TFORF0295 | MEF2A | NM_001319206, XM_011521583, ENST00000557942 | 385 | 3935 | 7485 | None |
| Genewiz | TFORF0296 | MEF2A | XM_011521587, NM_001130928, ENST00000449277 | 386 | 3936 | 7486 | None |
| Genewiz | TFORF0297 | CREM | NM_182720, ENST00000356917 | 387 | 3937 | 7487 | None |
| Genewiz | TFORF0298 | CREM | NM_182717, ENST00000473940 | 388 | 3938 | 7488 | None |
| Genewiz | TFORF0299 | CREM | NM_001267570, ENST00000468236 | 389 | 3939 | 7489 | None |
| Genewiz | TFORF0300 | CREM | XM_017015722, NM_183060, ENST00000374728, | 390 | 3940 | 7490 | None |
| | | | ENST00000348787 | | | | |
| Genewiz | TFORF0301 | CREM | XM_006717378, NM_181571, ENST00000345491 | 391 | 3941 | 7491 | None |
| Genewiz | TFORF0302 | CREM | NM_182718, ENST00000488328 | 392 | 3942 | 7492 | None |
| Genewiz | TFORF0303 | CREM | NM_182769, ENST00000342105 | 393 | 3943 | 7493 | None |
| Genewiz | TFORF0304 | CREM | NM_182719, ENST00000487763 | 394 | 3944 | 7494 | None |
| Genewiz | TFORF0305 | CREM | NM_182721, ENST00000474931 | 395 | 3945 | 7495 | None |
| Genewiz | TFORF0306 | CREM | NM_001267564, ENST00000395887 | 396 | 3946 | 7496 | None |
| Genewiz | TFORF0307 | CREM | NM_182771, ENST00000361599 | 397 | 3947 | 7497 | None |
| Genewiz | TFORF0308 | CREM | XM_006717383, NM_183012, ENST00000374734 | 398 | 3948 | 7498 | None |
| Genewiz | TFORF0309 | CREM | NM_182724, ENST00000490511 | 399 | 3949 | 7499 | None |
| Genewiz | TFORF0310 | CREM | NM_183013, ENST00000354759, ENST00000439705 | 400 | 3950 | 7500 | None |
| Genewiz | TFORF0311 | CREM | XM_011519324, XM_011519325, ENST00000479070 | 401 | 3951 | 7501 | None |
| Genewiz | TFORF0312 | CREM | XM_006717379, NM_183011, ENST00000337656 | 402 | 3952 | 7502 | None |
| Genewiz | TFORF0313 | CREM | NM_001267567, ENST00000463314 | 403 | 3953 | 7503 | None |
| Genewiz | TFORF0314 | CREM | NM_001881, ENST00000374726, ENST00000489321 | 404 | 3954 | 7504 | None |
| Genewiz | TFORF0315 | CREM | NM_182723, ENST00000488741 | 405 | 3955 | 7505 | None |
| Genewiz | TFORF0316 | MEF2D | XM_006711333, NM_001271629, XM_017001314, | 406 | 3956 | 7506 | None |
| | | | XM_017001315, ENST00000360595 | | | | |
| Genewiz | TFORF0317 | MEF2D | XM_006711334, ENST00000464356 | 407 | 3957 | 7507 | None |
| Genewiz | TFORF0318 | ZNF117 | NM_015852, ENST00000282869, ENST00000620222 | 408 | 3958 | 7508 | None |
| Genewiz | TFORF0319 | MSLN | XM_017022857, XM_011522348, NM_013404, | 409 | 3959 | 7509 | None |
| | | | ENST00000382862 | | | | |
| Genewiz | TFORF0320 | MSLN | NM_001177355, NM_005823, ENST00000563941, | 410 | 3960 | 7510 | None |
| | | | ENST00000545450, ENST00000566549 | | | | |
| Genewiz | TFORF0321 | ZNF112 | NM_001083335, ENST00000337401 | 411 | 3961 | 7511 | None |
| Genewiz | TFORF0322 | ZNF112 | NM_013380, ENST00000354340 | 412 | 3962 | 7512 | None |
| Genewiz | TFORF0323 | LCORL | XM_011513822, ENST00000635767 | 413 | 3963 | 7513 | None |
| Genewiz | TFORF0324 | LCORL | NM_153686, ENST00000326877 | 414 | 3964 | 7514 | None |
| Genewiz | TFORF0325 | LCORL | NM_001166139, ENST00000382226 | 415 | 3965 | 7515 | None |
| Genewiz | TFORF0326 | ZNF337 | NM_015655, XM_006723558, XM_011529219, | 416 | 3966 | 7516 | None |
| | | | NM_001290261, ENST00000376436, ENST00000252979 | | | | |
| Genewiz | TFORF0327 | ZNF334 | XM_011528892, XM_017027934, ENST00000593880 | 417 | 3967 | 7517 | None |
| Genewiz | TFORF0328 | ZNF334 | XM_017027938, NM_018102, XM_017027936, | 418 | 3968 | 7518 | None |
| | | | ENST00000347606 | | | | |
| Genewiz | TFORF0329 | ZNF334 | XM_017027944, ENST00000457685 | 419 | 3969 | 7519 | None |
| Genewiz | TFORF0330 | ZNF334 | NM_001270497, XM_011528897, XM_017027941, | 420 | 3970 | 7520 | None |
| | | | XM_017027939, XM_017027940, ENST00000615481, | | | | |
| | | | ENST00000625284 | | | | |
| Genewiz | TFORF0331 | ZNF335 | NM_022095, ENST00000322927 | 421 | 3971 | 7521 | None |
| Genewiz | TFORF0332 | ZNF333 | NM_001300912, ENST00000540689 | 422 | 3972 | 7522 | None |
| Genewiz | TFORF0333 | ZNF333 | XM_011528362, NM_032433, XM_017027367, | 423 | 3973 | 7523 | None |
| | | | ENST00000292530 | | | | |
| Genewiz | TFORF0334 | ZNF331 | XM_017026937, XM_017026936, XM_011527078, | 424 | 3974 | 7524 | None |
| | | | XM_011527076, NM_001317120, NM_018555, | | | | |
| | | | XM_017026939, XM_017026938, NM_001317114, | | | | |
| | | | NM_001317113, NM_001079906, NM_001317115, | | | | |
| | | | NM_001253799, NM_001253798, XM_017026940, | | | | |
| | | | NM_001253800, NM_001253801, NM_001317117, | | | | |
| | | | NM_001317116, NM_001317118, NM_001317121, | | | | |
| | | | NM_001317119, NM_001079907, ENST00000253144, | | | | |
| | | | ENST00000511593, ENST00000449416, ENST00000411977, | | | | |
| | | | ENST00000511154, ENST0513999, ENST00000512387 | | | | |
| Genewiz | TFORF0335 | ZNF91 | NM_001300951, ENST00000397082 | 425 | 3975 | 7525 | None |
| Genewiz | TFORF0336 | ZNF91 | NM_003430, ENST00000300619 | 426 | 3976 | 7526 | None |
| Genewiz | TFORF0337 | MECOM | NM_001205194, NM_005241, NM_001105078, | 427 | 3977 | 7527 | None |
| | | | XM_005247223, ENST00000628990, ENST00000468789 | | | | |
| Genewiz | TFORF0338 | MECOM | NM_001105077, XM_017005874, ENST00000264674 | 428 | 3978 | 7528 | None |
| Genewiz | TFORF0339 | MECOM | NM_001164000, XM_017005875, ENST00000464456 | 429 | 3979 | 7529 | None |
| Genewiz | TFORF0340 | MECOM | XM_005247220, XM_005247221, XM_005247219, | 430 | 3980 | 7530 | None |
| | | | ENST00000472280, ENST00000433243 | | | | |
| Genewiz | TFORF0341 | MECOM | XM_005247215, ENST00000494292 | 431 | 3981 | 7531 | None |
| Genewiz | TFORF0342 | ETF1 | NM_001256302, NM_001291975, NM_001291974, | 432 | 3982 | 7532 | None |
| | | | XM_005271921, ENST00000499810 | | | | |
| Genewiz | TFORF0343 | ETF1 | NM_004730, ENST00000360541 | 433 | 3983 | 7533 | None |
| Genewiz | TFORF0344 | ETF1 | NM_001282185, ENST00000503014 | 434 | 3984 | 7534 | None |
| Genewiz | TFORF0345 | MYT1L | NM_015025, XM_017003614, XM_017003613, | 435 | 3985 | 7535 | None |
| | | | ENST00000428368 | | | | |
| Genewiz | TFORF0346 | MYT1L | NM_001303052, XM_017003609, XM_017003610, | 436 | 3986 | 7536 | None |
| | | | ENST00000399161 | | | | |
| Genewiz | TFORF0347 | FOXB1 | NM_012182, ENST00000396057 | 437 | 3987 | 7537 | None |
| Genewiz | TFORF0348 | FOXB2 | NM_001013735, ENST00000376708 | 438 | 3988 | 7538 | None |
| Genewiz | TFORF0349 | ZNF48 | NM_001214907, ENST00000622647 | 439 | 3989 | 7539 | None |
| Genewiz | TFORF0350 | ZNF48 | NM_001214906, NM_001214909, NM_152652, | 440 | 3990 | 7540 | None |
| | | | ENST00000613509, ENST00000320159 | | | | |
| Genewiz | TFORF0351 | FOXL2 | NM_023067, ENST00000330315 | 441 | 3991 | 7541 | None |
| Genewiz | TFORF0352 | FOXL1 | NM_005250, ENST00000320241 | 442 | 3992 | 7542 | None |
| Genewiz | TFORF0353 | ZNF33B | NM_006955, ENST00000359467 | 443 | 3993 | 7543 | None |
| Genewiz | TFORF0354 | ZNF33A | NM_001278170, ENST00000628825 | 444 | 3994 | 7544 | None |
| Genewiz | TFORF0355 | ZNF33A | XM_011519650, NM_006954, ENST00000432900 | 445 | 3995 | 7545 | None |
| Genewiz | TFORF0356 | ZNF33A | XM_011519651, NM_006974, ENST00000458705 | 446 | 3996 | 7546 | None |
| Genewiz | TFORF0357 | ZNF33A | NM_001278173, ENST00000307441 | 447 | 3997 | 7547 | None |
| Genewiz | TFORF0358 | ZNF33A | NM_001324175, NM_001278174, NM_001278179, | 448 | 3998 | 7548 | None |
| | | | NM_001278171, NM_001324176, NM_001278178, | | | | |
| | | | NM_001324177, ENST00000374618 | | | | |
| Genewiz | TFORF0359 | TUB | NM_003320, ENST00000305253 | 449 | 3999 | 7549 | None |
| Genewiz | TFORF0360 | TUB | NM_177972, ENST00000299506 | 450 | 4000 | 7550 | None |
| Genewiz | TFORF0361 | TCEB3 | NM_003198, ENST00000418390, ENST00000613537 | 451 | 4001 | 7551 | None |
| Genewiz | TFORF0362 | TCEB2 | NM_207013, ENST00000262306 | 452 | 4002 | 7552 | None |
| Genewiz | TFORF0363 | TCEB2 | NM_007108, ENST00000409906 | 453 | 4003 | 7553 | None |
| Genewiz | TFORF0364 | TCEB1 | NM_001204861, NM_001204862, NM_001204858, | 454 | 4004 | 7554 | None |
| | | | NM_001204859, NM_005648, NM_001204860, | | | | |
| | | | NM_001204857, XM_011517580, XM_011517581, | | | | |
| | | | ENST00000518127, ENST00000622804, ENST00000520242, | | | | |
| | | | ENST00000519487, ENST00000284811, ENST00000522337, | | | | |
| | | | ENST00000523815 | | | | |
| Genewiz | TFORF0365 | TCEB1 | NM_001204863, NM_001204864, ENST00000520210 | 455 | 4005 | 7555 | None |
| Genewiz | TFORF0366 | NFYC | XM_017001365, XM_005270894, XM_011541516, | 456 | 4006 | 7556 | None |
| | | | XM_005270895, NM_001308114, ENST00000308733 | | | | |
| Genewiz | TFORF0367 | NFYC | XM_006710661, NM_001308115, XM_006710658, | 457 | 4007 | 7557 | None |
| | | | ENST00000372652 | | | | |
| Genewiz | TFORF0368 | NFYC | NM_001142589, ENST00000427410 | 458 | 4008 | 7558 | None |
| Genewiz | TFORF0369 | NFYC | NM_001142587, ENST00000456393 | 459 | 4009 | 7559 | None |
| Genewiz | TFORF0370 | NFYC | NM_001142588, XM_017001367, XM_006710660, | 460 | 4010 | 7560 | None |
| | | | ENST00000425457 | | | | |
| Genewiz | TFORF0371 | NFYC | NM_001142590, ENST00000372653 | 461 | 4011 | 7561 | None |
| Genewiz | TFORF0372 | NFYB | NM_006166, ENST00000240055, ENST00000551727 | 462 | 4012 | 7562 | None |
| Genewiz | TFORF0373 | NFYA | NM_002505, ENST00000341376 | 463 | 4013 | 7563 | None |
| Genewiz | TFORF0374 | NFYA | NM_021705, ENST00000353205 | 464 | 4014 | 7564 | None |
| Genewiz | TFORF0375 | MYBL1 | NM_001080416, ENST00000522677 | 465 | 4015 | 7565 | None |
| Genewiz | TFORF0376 | SMARCA1 | NM_001282875, ENST00000371123 | 466 | 4016 | 7566 | None |
| Genewiz | TFORF0377 | SMARCA1 | NM_001282874, ENST00000371121 | 467 | 4017 | 7567 | None |
| Genewiz | TFORF0378 | SMARCA1 | NM_003069, XM_005262461, ENST00000371122 | 468 | 4018 | 7568 | None |
| Genewiz | TFORF0379 | SMARCA2 | NM_001289400, ENST00000302401 | 469 | 4019 | 7569 | None |
| Genewiz | TFORF0380 | SMARCA2 | NM_001289399, ENST00000382185, ENST00000417599 | 470 | 4020 | 7570 | None |
| Genewiz | TFORF0381 | SMARCA2 | NM_139045, ENST00000357248, ENST00000382194 | 471 | 4021 | 7571 | None |
| Genewiz | TFORF0382 | SMARCA2 | NM_001289397, ENST00000450198 | 472 | 4022 | 7572 | None |
| Genewiz | TFORF0383 | SMARCA2 | NM_001289398, ENST00000324954 | 473 | 4023 | 7573 | None |
| Genewiz | TFORF0384 | SMARCA2 | NM_003070, NM_001289396, ENST00000349721, | 474 | 4024 | 7574 | None |
| | | | ENST00000382203 | | | | |
| Genewiz | TFORF0385 | SMARCA4 | XM_017027167, NM_001128847, ENST00000590574 | 475 | 4025 | 7575 | None |
| Genewiz | TFORF0386 | SMARCA4 | XM_017027168, NM_001128848, ENST00000444061 | 476 | 4026 | 7576 | None |
| Genewiz | TFORF0387 | SMARCA4 | XM_017027165, NM_001128845, ENST00000541122 | 477 | 4027 | 7577 | None |
| Genewiz | TFORF0388 | SMARCA4 | XM_006722845, XM_011528198, NM_001128849, | 478 | 4028 | 7578 | None |
| | | | XM_006722846, ENST00000450717 | | | | |
| Genewiz | TFORF0389 | SMARCA4 | NM_003072, NM_001128844, ENST00000344626, | 479 | 4029 | 7579 | None |
| | | | ENST00000429416 | | | | |
| Genewiz | TFORF0390 | SMARCA4 | XM_017027166, NM_001128846, ENST00000589677 | 480 | 4030 | 7580 | None |
| Genewiz | TFORF0391 | FERD3L | NM_152898, ENST00000275461 | 481 | 4031 | 7581 | None |
| Genewiz | TFORF0392 | ZNF7 | NM_003416, XM_011517293, XM_011517294, | 482 | 4032 | 7582 | None |
| | | | ENST00000528372 | | | | |
| Genewiz | TFORF0393 | ZNF7 | NM_001282795, XM_011517292, ENST00000446747 | 483 | 4033 | 7583 | None |
| Genewiz | TFORF0394 | ZNF7 | XM_017013817, ENST00000325217 | 484 | 4034 | 7584 | None |
| Genewiz | TFORF0395 | ZNF7 | NM_001282796, ENST00000525266 | 485 | 4035 | 7585 | None |
| Genewiz | TFORF0396 | ZNF7 | NM_001282797, XM_011517297, XM_006716654, | 486 | 4036 | 7586 | None |
| | | | XM_006716656, ENST00000544249 | | | | |
| Genewiz | TFORF0397 | ZNF2 | NM_001291604, ENST00000611463 | 487 | 4037 | 7587 | None |
| Genewiz | TFORF0398 | ZNF2 | NM_001291605, ENST00000611147 | 488 | 4038 | 7588 | None |
| Genewiz | TFORF0399 | ZNF2 | NM_001017396, ENST00000617923 | 489 | 4039 | 7589 | None |
| Genewiz | TFORF0400 | ZNF2 | NM_001282398, ENST00000622059 | 490 | 4040 | 7590 | None |
| Genewiz | TFORF0401 | ZNF2 | NM_021088, ENST00000614034 | 491 | 4041 | 7591 | None |
| Genewiz | TFORF0402 | ZNF3 | NM_017715, ENST00000413658 | 492 | 4042 | 7592 | None |
| Genewiz | TFORF0403 | ZNF3 | NM_032924, NM_001318135, NM_001278290, | 493 | 4043 | 7593 | None |
| | | | NM_001278284, NM_001278287, ENST00000424697, | | | | |
| | | | ENST00000303915, ENST00000299667 | | | | |
| Genewiz | TFORF0404 | CTCFL | NM_001269043, ENST00000423479 | 494 | 4044 | 7594 | None |
| Genewiz | TFORF0405 | CTCFL | NM_001269041, NM_080618, NM_001269040, | 495 | 4045 | 7595 | None |
| | | | ENST00000608263, ENST00000609232, ENST00000243914, | | | | |
| | | | ENST00000371196 | | | | |
| Genewiz | TFORF0406 | CTCFL | NM_001269052, NM_001269051, ENST00000608158, | 496 | 4046 | 7596 | None |
| | | | ENST00000481655 | | | | |
| Genewiz | TFORF0407 | CTCFL | NM_001269049, ENST00000433949 | 497 | 4047 | 7597 | None |
| Genewiz | TFORF0408 | CTCFL | NM_001269048, ENST00000432255 | 498 | 4048 | 7598 | None |
| Genewiz | TFORF0409 | CTCFL | NM_001269046, ENST00000429804 | 499 | 4049 | 7599 | None |
| Genewiz | TFORF0410 | CTCFL | NM_001269055, ENST00000608903 | 500 | 4050 | 7600 | None |
| Genewiz | TFORF0411 | CTCFL | NM_001269054, ENST00000502686 | 501 | 4051 | 7601 | None |
| Genewiz | TFORF0412 | CTCFL | NM_001269044, ENST00000608440 | 502 | 4052 | 7602 | None |
| Genewiz | TFORF0413 | CTCFL | NM_001269045, ENST00000608425 | 503 | 4053 | 7603 | None |
| Genewiz | TFORF0414 | CTCFL | NM_001269050, ENST00000539382 | 504 | 4054 | 7604 | None |
| Genewiz | TFORF0415 | CTCFL | NM_001269047, ENST00000422869 | 505 | 4055 | 7605 | None |
| Genewiz | TFORF0416 | ZNF559- | NM_001202425, ENST00000446085, ENST00000602856 | 506 | 4056 | 7606 | None |
| | | ZNF177 | | | | | |
| Genewiz | TFORF0417 | SULT2A1 | NM_003167, ENST00000222002 | 507 | 4057 | 7607 | None |
| Genewiz | TFORF0418 | CDC5L | NM_001253, ENST00000371477 | 508 | 4058 | 7608 | None |
| Genewiz | TFORF0419 | SKIL | NM_001145098, ENST00000426052 | 509 | 4059 | 7609 | None |
| Genewiz | TFORF0420 | SKIL | NM_001145097, ENST00000413427 | 510 | 4060 | 7610 | None |
| Genewiz | TFORF0421 | SKIL | XM_006713735, NM_005414, XM_005247721, | 511 | 4061 | 7611 | None |
| | | | NM_001248008, ENST00000259119, ENST00000458537 | | | | |
| Genewiz | TFORF0422 | PBX2 | NM_002586, ENST00000375050 | 512 | 4062 | 7612 | None |
| Genewiz | TFORF0423 | PBX3 | NM_001134778, ENST00000447726 | 513 | 4063 | 7613 | None |
| Genewiz | TFORF0424 | PBX3 | NM_006195, ENST00000373489 | 514 | 4064 | 7614 | None |
| Genewiz | TFORF0425 | PBX3 | XM_006717132, ENST00000342287 | 515 | 4065 | 7615 | None |
| Genewiz | TFORF0426 | PBX3 | XM_006717130, ENST00000373487 | 516 | 4066 | 7616 | None |
| Genewiz | TFORF0427 | PBX1 | NM_002585, XM_005245229, ENST00000420696 | 517 | 4067 | 7617 | None |
| Genewiz | TFORF0428 | PBX1 | NM_001204963, ENST00000627490 | 518 | 4068 | 7618 | None |
| Genewiz | TFORF0429 | PBX1 | NM_001204961, XM_017001396, ENST00000367897 | 519 | 4069 | 7619 | None |
| Genewiz | TFORF0430 | ZNF883 | NM_001101338, ENST00000619044 | 520 | 4070 | 7620 | None |
| Genewiz | TFORF0431 | AIRE | NM_000383, ENST00000291582 | 521 | 4071 | 7621 | None |
| Genewiz | TFORF0432 | ARHGAP35 | XM_017026714, NM_004491, ENST00000614079, | 522 | 4072 | 7622 | None |
| | | | ENST00000404338 | | | | |
| Genewiz | TFORF0433 | ZSCAN32 | NM_001324340, NM_001324342, NM_001324344, | 523 | 4073 | 7623 | None |
| | | | NM_001324341, NM_001324345, NM_017810, | | | | |
| | | | ENST00000304926 | | | | |
| Genewiz | TFORF0434 | ZSCAN32 | NM_001284527, XM_011522555, XM_017023371, | 524 | 4074 | 7624 | None |
| | | | ENST00000396846, ENST00000396852 | | | | |
| Genewiz | TFORF0435 | ZSCAN32 | NM_001284528, NM_001284529, ENST00000618425, | 525 | 4075 | 7625 | None |
| | | | ENST00000439568 | | | | |
| Genewiz | TFORF0436 | ZSCAN31 | NM_001135216, NM_030899, NM_001243241, | 526 | 4076 | 7626 | None |
| | | | NM_145909, NM_001135215, XM_011514809, | | | | |
| | | | XM_011514811, XM_011514813, XM_011514812, | | | | |
| | | | XM_005249295, XM_011514807, XM_017011196, | | | | |
| | | | XM_005249296, XM_011514808, ENST00000439158, | | | | |
| | | | ENST00000396838, ENST00000414429, ENST00000344279 | | | | |
| Genewiz | TFORF0437 | ZSCAN31 | NM_001243243, NM_001243242, NM_001243244, | 527 | 4077 | 7627 | None |
| | | | ENST00000611469, ENST00000446474 | | | | |
| Genewiz | TFORF0438 | ZSCAN30 | NM_001166012, NM_001112734, XM_006722371, | 528 | 4078 | 7628 | None |
| | | | XM_017025515, XM_005258183, ENST00000333206, | | | | |
| | | | ENST00000420878 | | | | |
| Genewiz | TFORF0439 | ZSCAN30 | NM_001288711, XM_011525789, XM_017025522, | 529 | 4079 | 7629 | None |
| | | | XM_017025519, XM_017025520, XM_017025521, | | | | |
| | | | ENST00000610712 | | | | |
| Genewiz | TFORF0440 | ZFY | NM_001145276, ENST00000625061 | 530 | 4080 | 7630 | None |
| Genewiz | TFORF0441 | ZEY | XM_005262570, XM_017030075, NM_003411, | 531 | 4081 | 7631 | None |
| | | | ENST00000383052, ENST00000155093 | | | | |
| Genewiz | TFORF0442 | ZFX | XM_011545578, XM_017029792, XM_017029791, | 532 | 4082 | 7632 | None |
| | | | XM_017029790, XM_006724513, XM_017029789, | | | | |
| | | | XM_017029788, XM_017029793, ENST00000379188 | | | | |
| Genewiz | TFORF0443 | ZFX | NM_001178086, ENST00000539115 | 533 | 4083 | 7633 | None |
| Genewiz | TFORF0444 | ZFX | XM_005274592, XM_011545581, XM_005274591, | 534 | 4084 | 7634 | None |
| | | | NM_001178084, NM_001178085, NM_003410, | | | | |
| | | | XM_017029794, XM_011545579, XM_017029795, | | | | |
| | | | ENST00000379177, ENST00000304543 | | | | |
| Genewiz | TFORF0445 | ZNF805 | NM_001145078, ENST00000354309 | 535 | 4085 | 7635 | None |
| Genewiz | TFORF0446 | ZNF805 | NM_001023563, ENST00000414468 | 536 | 4086 | 7636 | None |
| Genewiz | TFORF0447 | TOPORS | NM_001195622, ENST00000379858 | 537 | 4087 | 7637 | None |
| Genewiz | TFORF0448 | TOPORS | NM_005802, ENST00000360538 | 538 | 4088 | 7638 | None |
| Genewiz | TFORF0449 | DNMT1 | NM_001130823, ENST00000359526 | 539 | 4089 | 7639 | None |
| Genewiz | TFORF0450 | DNMT1 | NM_001379, ENST00000340748 | 540 | 4090 | 7640 | None |
| Genewiz | TFORF0451 | THRA | NM_199334, ENST00000450525, ENST00000546243 | 541 | 4091 | 7641 | None |
| Genewiz | TFORF0452 | THRA | NM_001190918, ENST00000584985 | 542 | 4092 | 7642 | None |
| Genewiz | TFORF0453 | THRA | NM_001190919, NM_003250, ENST00000394121, | 543 | 4093 | 7643 | None |
| | | | ENST00000264637 | | | | |
| Genewiz | TFORF0454 | TAF4B | NM_005640, ENST00000269142 | 544 | 4094 | 7644 | None |
| Genewiz | TFORF0455 | TAF4B | NM_001293725, ENST00000578121 | 545 | 4095 | 7645 | None |
| Genewiz | TFORF0456 | HOXD9 | NM_014213, ENST00000249499 | 546 | 4096 | 7646 | None |
| Genewiz | TFORF0457 | TRIM24 | NM_015905, ENST00000343526 | 547 | 4097 | 7647 | None |
| Genewiz | TFORF0458 | TRIM24 | NM_003852, ENST00000415680 | 548 | 4098 | 7648 | None |
| Genewiz | TFORF0459 | CREB5 | NM_182899, ENST00000396299, ENST00000409603 | 549 | 4099 | 7649 | None |
| Genewiz | TFORF0460 | CREB5 | XM_017012808, NM_182898, ENST00000357727 | 550 | 4100 | 7650 | None |
| Genewiz | TFORF0461 | CREB5 | XM_005249906, XM_017012806, NM_004904, | 551 | 4101 | 7651 | None |
| | | | ENST00000396300 | | | | |
| Genewiz | TFORF0462 | CREB5 | NM_001011666, ENST00000396298 | 552 | 4102 | 7652 | None |
| Genewiz | TFORF0463 | HOXD1 | NM_024501, ENST00000331462 | 553 | 4103 | 7653 | None |
| Genewiz | TFORF0464 | TRIM22 | NM_006074, ENST00000379965 | 554 | 4104 | 7654 | None |
| Genewiz | TFORF0465 | NKX1-1 | NM_001290079 | 555 | 4105 | 7655 | None |
| Genewiz | TFORF0466 | SLC15A1 | NM_005073, ENST00000376503 | 556 | 4106 | 7656 | None |
| Genewiz | TFORF0467 | CARHSP1 | NM_001278265, NM_001278262, NM_001278266, | 557 | 4107 | 7657 | None |
| | | | NM_001278264, NM_001278263, NM_001042476, | | | | |
| | | | NM_001278261, NM_001278260, NM_014316, | | | | |
| | | | XM_005255229, XM_011522444, ENST00000396593, | | | | |
| | | | ENST0610831, ENST00000614449, ENST00000619881, | | | | |
| | | | ENST00000618335, ENST00000611932, ENST00000311052, | | | | |
| | | | ENST00000561530, ENST00000567554 | | | | |
| Genewiz | TFORF0468 | KAT7 | NM_001199158, ENST00000510819 | 558 | 4108 | 7658 | None |
| Genewiz | TFORF0469 | KAT7 | NM_001199157, ENST00000509773 | 559 | 4109 | 7659 | None |
| Genewiz | TFORF0470 | KAT7 | NM_001199155, ENST00000424009 | 560 | 4110 | 7660 | None |
| Genewiz | TFORF0471 | KAT7 | NM_001199156, ENST00000454930 | 561 | 4111 | 7661 | None |
| Genewiz | TFORF0472 | ZNF221 | XM_017027232, NM_001297588, NM_001297589, | 562 | 4112 | 7662 | None |
| | | | NM_013359, ENST00000587682, ENST00000251269, | | | | |
| | | | ENST00000592350, ENST00000622072 | | | | |
| Genewiz | TFORF0473 | SP140 | NM_001278452, ENST00000343805 | 563 | 4113 | 7663 | None |
| Genewiz | TFORF0474 | SP140 | NM_007237, ENST00000392045 | 564 | 4114 | 7664 | None |
| Genewiz | TFORF0475 | SP140 | NM_001005176, ENST00000373645 | 565 | 4115 | 7665 | None |
| Genewiz | TFORF0476 | SP140 | NM_001278453, ENST00000417495 | 566 | 4116 | 7666 | None |
| Genewiz | TFORF0477 | SP140 | NM_001278451, ENST00000420434 | 567 | 4117 | 7667 | None |
| Genewiz | TFORF0478 | ZNF222 | NM_013360, ENST00000187879 | 568 | 4118 | 7668 | None |
| Genewiz | TFORF0479 | ZNF222 | NM_001129996, ENST00000391960 | 569 | 4119 | 7669 | None |
| Genewiz | TFORF0480 | HIF1A | NM_001243084, ENST00000539097 | 570 | 4120 | 7670 | None |
| Genewiz | TFORF0481 | HIF1A | NM_181054, ENST00000323441 | 571 | 4121 | 7671 | None |
| Genewiz | TFORF0482 | DMRTB1 | NM_033067, ENST00000371445 | 572 | 4122 | 7672 | None |
| Genewiz | TFORF0483 | ZNF223 | XM_017027258, NM_013361, XM_017027259, | 573 | 4123 | 7673 | None |
| | | | ENST00000434772 | | | | |
| Genewiz | TFORF0484 | ZFP92 | XM_011531115, XM_005274652, NM_001136273, | 574 | 4124 | 7674 | None |
| | | | ENST00000338647 | | | | |
| Genewiz | TFORF0485 | ZNF852 | NM_001287349 | 575 | 4125 | 7675 | None |
| Genewiz | TFORF0486 | ZFP90 | NM_001305204, ENST00000611381 | 576 | 4126 | 7676 | None |
| Genewiz | TFORF0487 | ZFP90 | NM_133458, NM_001305203, XM_005255804, | 577 | 4127 | 7677 | None |
| | | | ENST00000570495, ENST00000563169, ENST00000398253 | | | | |
| Genewiz | TFORF0488 | ZFP90 | NM_001305208, NM_001305206, ENST00000564323 | 578 | 4128 | 7678 | None |
| Genewiz | TFORF0489 | ZFP91 | NM_053023, ENST00000316059 | 579 | 4129 | 7679 | None |
| Genewiz | TFORF0490 | ZSCAN4 | XM_011526607, XM_017026458, NM_152677, | 580 | 4130 | 7680 | None |
| | | | ENST00000318203, ENST00000612521 | | | | |
| Genewiz | TFORF0491 | ZSCAN1 | NM_182572, ENST00000282326 | 581 | 4131 | 7681 | None |
| Genewiz | TFORF0492 | ZSCAN2 | NM_001007072, ENST00000379358 | 582 | 4132 | 7682 | None |
| Genewiz | TFORF0493 | ZSCAN2 | NM_181877, ENST00000448803, ENST00000546148, | 583 | 4133 | 7683 | None |
| | | | ENST00000540894 | | | | |
| Genewiz | TFORF0494 | ZSCAN2 | NM_017894, ENST00000334141 | 584 | 4134 | 7684 | None |
| Genewiz | TFORF0495 | EGR2 | NM_000399, NM_001136177, NM_001136178, | 585 | 4135 | 7685 | None |
| | | | ENST00000242480, ENST00000439032 | | | | |
| Genewiz | TFORF0496 | EGR2 | NM_001136179, NM_001321037, ENST00000411732 | 586 | 4136 | 7686 | None |
| Genewiz | TFORF0497 | EGR3 | NM_004430, ENST00000317216 | 587 | 4137 | 7687 | None |
| Genewiz | TFORF0498 | EGR3 | NM_001199880, XM_005273426, ENST00000522910 | 588 | 4138 | 7688 | None |
| Genewiz | TFORF0499 | PPARGC1A | XM_011513770, XM_011513771, ENST00000613098 | 589 | 4139 | 7689 | None |
| Genewiz | TFORF0500 | PPARGC1A | NM_013261, ENST00000264867 | 590 | 4140 | 7690 | None |
| Genewiz | TFORF0501 | EGR4 | NM_001965, ENST00000545030 | 591 | 4141 | 7691 | None |
| Genewiz | TFORF0502 | NFE4 | NM_001085386, ENST00000638942 | 592 | 4142 | 7692 | None |
| Genewiz | TFORF0503 | RNF138 | XM_005258286, NM_198128, ENST00000257190 | 593 | 4143 | 7693 | None |
| Genewiz | TFORF0504 | ZFP30 | NM_001320666, NM_001320667, NM_014898, | 594 | 4144 | 7694 | None |
| | | | NM_001320669, NM_001320668, ENST00000351218, | | | | |
| | | | ENST00000392144, ENST00000514101 | | | | |
| Genewiz | TFORF0505 | CDK2 | NM_001290230, ENST00000440311 | 595 | 4145 | 7695 | None |
| Genewiz | TFORF0506 | CDK2 | NM_052827, ENST00000354056 | 596 | 4146 | 7696 | None |
| Genewiz | TFORF0507 | CDK2 | XM_011537732, ENST00000553376 | 597 | 4147 | 7697 | None |
| Genewiz | TFORF0508 | TSHZ2 | NM_001193421, ENST00000603338 | 598 | 4148 | 7698 | None |
| Genewiz | TFORF0509 | TSHZ2 | NM_173485, XM_017027640, ENST00000371497 | 599 | 4149 | 7699 | None |
| Genewiz | TFORF0510 | TSHZ3 | NM_020856, ENST00000240587 | 600 | 4150 | 7700 | None |
| Genewiz | TFORF0511 | TSHZ1 | NM_005786, XM_005266641, ENST00000322038 | 601 | 4151 | 7701 | None |
| Genewiz | TFORF0512 | TSHZ1 | NM_001308210, ENST00000580243 | 602 | 4152 | 7702 | None |
| Genewiz | TFORF0513 | PAX5 | NM_001280550, ENST00000520154 | 603 | 4153 | 7703 | None |
| Genewiz | TFORF0514 | PAX5 | NM_001280553, ENST00000520281 | 604 | 4154 | 7704 | None |
| Genewiz | TFORF0515 | PAX5 | NM_001280548, ENST00000377853 | 605 | 4155 | 7705 | None |
| Genewiz | TFORF0516 | PAX5 | NM_001280549, ENST00000523241 | 606 | 4156 | 7706 | None |
| Genewiz | TFORF0517 | PAX5 | NM_016734, ENST00000358127 | 607 | 4157 | 7707 | None |
| Genewiz | TFORF0518 | PAX5 | NM_001280547, ENST00000377852 | 608 | 4158 | 7708 | None |
| Genewiz | TFORF0519 | PAX5 | NM_001280554, ENST00000414447 | 609 | 4159 | 7709 | None |
| Genewiz | TFORF0520 | PAX5 | NM_001280555, ENST00000446742 | 610 | 4160 | 7710 | None |
| Genewiz | TFORF0521 | PAX5 | NM_001280556, ENST00000522003 | 611 | 4161 | 7711 | None |
| Genewiz | TFORF0522 | PAX5 | NM_001280551, ENST00000523145 | 612 | 4162 | 7712 | None |
| Genewiz | TFORF0523 | PAX5 | NM_001280552, ENST00000377847 | 613 | 4163 | 7713 | None |
| Genewiz | TFORF0524 | PAX4 | XM_011516276, ENST00000639438 | 614 | 4164 | 7714 | None |
| Genewiz | TFORF0525 | PAX4 | NM_006193, ENST00000341640 | 615 | 4165 | 7715 | None |
| Genewiz | TFORF0526 | PAX7 | NM_001135254, ENST00000420770 | 616 | 4166 | 7716 | None |
| Genewiz | TFORF0527 | PAX7 | NM_002584, ENST00000375375 | 617 | 4167 | 7717 | None |
| Genewiz | TFORF0528 | PAX6 | NM_001604, NM_001258463, NM_001310158, | 618 | 4168 | 7718 | None |
| | | | NM_001258462, ENST00000606377, ENST00000640368, | | | | |
| | | | ENST00000419022, ENST00000379129, ENST00000379107, | | | | |
| | | | ENST00000638903, ENST00000639409, ENST00000640975 | | | | |
| Genewiz | TFORF0529 | PAX6 | NM_001310160, NM_001310161, ENST00000638629, | 619 | 4169 | 7719 | None |
| | | | ENST00000639548, ENST00000640125, ENST00000481563, | | | | |
| | | | ENST00000639386 | | | | |
| Genewiz | TFORF0530 | PAX6 | NM_001310159, ENST00000639034 | 620 | 4170 | 7720 | None |
| Genewiz | TFORF0531 | PAX6 | NM_001258465, NM_000280, NM_001258464, | 621 | 4171 | 7721 | None |
| | | | NM_001127612, ENST00000638914, ENST00000640610, | | | | |
| | | | ENST00000379132, ENST00000379109, ENST00000639916, | | | | |
| | | | ENST00000241001 | | | | |
| Genewiz | TFORF0532 | PAX1 | NM_006192, ENST00000398485 | 622 | 4172 | 7722 | None |
| Genewiz | TFORF0533 | PAX1 | NM_001257096, ENST00000613128 | 623 | 4173 | 7723 | None |
| Genewiz | TFORF0534 | ZNF584 | NM_001318002, ENST00000322834 | 624 | 4174 | 7724 | None |
| Genewiz | TFORF0535 | ZNF584 | NM_173548, ENST00000306910 | 625 | 4175 | 7725 | None |
| Genewiz | TFORF0536 | PAX2 | NM_003988, ENST00000370296 | 626 | 4176 | 7726 | None |
| Genewiz | TFORF0537 | PAX2 | NM_003987, ENST00000428433 | 627 | 4177 | 7727 | None |
| Genewiz | TFORF0538 | PAX2 | NM_000278, ENST00000355243 | 628 | 4178 | 7728 | None |
| Genewiz | TFORF0539 | ZNF589 | NM_016089, ENST00000354698, ENST00000448461 | 629 | 4179 | 7729 | None |
| Genewiz | TFORF0540 | GCFC2 | NM_003203, ENST00000321027 | 630 | 4180 | 7730 | None |
| Genewiz | TFORF0541 | GCFC2 | NM_001201335, ENST00000470503 | 631 | 4181 | 7731 | None |
| Genewiz | TFORF0542 | ZFAT | NM_001029939, NM_001167583, NM_001289394, | 632 | 4182 | 7732 | None |
| | | | XM_011517203, ENST00000520727, ENST00000520214 | | | | |
| Genewiz | TFORF0543 | ZFAT | NM_001174158, ENST00000520356 | 633 | 4183 | 7733 | None |
| Genewiz | TFORF0544 | ZFAT | XM_011517206, ENST00000429442 | 634 | 4184 | 7734 | None |
| Genewiz | TFORF0545 | ZFAT | NM_020863, ENST00000377838 | 635 | 4185 | 7735 | None |
| Genewiz | TFORF0546 | ZFAT | NM_001174157, ENST00000523399 | 636 | 4186 | 7736 | None |
| Genewiz | TFORF0547 | PAX9 | NM_006194, ENST00000402703, ENST00000361487 | 637 | 4187 | 7737 | None |
| Genewiz | TFORF0548 | PAX8 | NM_013952, ENST00000348715 | 638 | 4188 | 7738 | None |
| Genewiz | TFORF0549 | PAX8 | NM_013992, ENST00000397647 | 639 | 4189 | 7739 | None |
| Genewiz | TFORF0550 | PAX8 | NM_013953, ENST00000263335 | 640 | 4190 | 7740 | None |
| Genewiz | TFORF0551 | ZNF611 | NM_030972, NM_001161499, NM_001161500, | 641 | 4191 | 7741 | None |
| | | | ENST00000540744, ENST00000543227, ENST00000319783 | | | | |
| Genewiz | TFORF0552 | ZNF611 | NM_001161501, ENST00000453741, ENST00000602162, | 642 | 4192 | 7742 | None |
| | | | ENST00000595798 | | | | |
| Genewiz | TFORF0553 | ZNF610 | NM_001161427, ENST00000601151, ENST00000613461 | 643 | 4193 | 7743 | None |
| Genewiz | TFORF0554 | ZNF610 | NM_001161426, NM_001161425, NM_173530, | 644 | 4194 | 7744 | None |
| | | | ENST00000321287, ENST00000403906, ENST00000327920, | | | | |
| | | | ENST00000616431 | | | | |
| Genewiz | TFORF0555 | ZNF613 | XM_017027315, NM_024840, ENST00000391794 | 645 | 4195 | 7745 | None |
| Genewiz | TFORF0556 | ZNF613 | XM_011527333, XM_005259269, NM_001031721, | 646 | 4196 | 7746 | None |
| | | | ENST00000293471 | | | | |
| Genewiz | TFORF0557 | ZNF615 | XM_011526825, XM_011526824, XM_011526826, | 647 | 4197 | 7747 | None |
| | | | NM_001199324, NM_001321319, NM_001321321, | | | | |
| | | | ENST00000594083, ENST00000598071, ENST00000618487 | | | | |
| Genewiz | TFORF0558 | ZNF615 | NM_001321317, ENST00000391795 | 648 | 4198 | 7748 | None |
| Genewiz | TFORF0559 | ZNF615 | XM_017026649, XM_017026648, NM_198480, | 649 | 4199 | 7749 | None |
| | | | NM_001321320, ENST00000602063, ENST00000376716 | | | | |
| Genewiz | TFORF0560 | ZNF614 | NM_025040, ENST00000270649 | 650 | 4200 | 7750 | None |
| Genewiz | TFORF0561 | ZNF616 | NM_178523, ENST00000600228 | 651 | 4201 | 7751 | None |
| Genewiz | TFORF0562 | ZNF619 | NM_001145083, ENST00000456778 | 652 | 4202 | 7752 | None |
| Genewiz | TFORF0563 | ZNF619 | NM_001145093, ENST00000432264 | 653 | 4203 | 7753 | None |
| Genewiz | TFORF0564 | ZNF619 | XM_017006225, XM_011533608, ENST00000429348, | 654 | 4204 | 7754 | None |
| | | | ENST00000522736 | | | | |
| Genewiz | TFORF0565 | ZNF619 | NM_001145082, ENST00000447116, ENST00000521353 | 655 | 4205 | 7755 | None |
| Genewiz | TFORF0566 | ZNF618 | NM_001318040, ENST00000615615 | 656 | 4206 | 7756 | None |
| Genewiz | TFORF0567 | ZNF618 | NM_133374, ENST00000288466 | 657 | 4207 | 7757 | None |
| Genewiz | TFORF0568 | ZNF618 | NM_001318042, ENST00000374126 | 658 | 4208 | 7758 | None |
| Genewiz | TFORF0569 | SRCAP | NM_006662, ENST00000262518 | 659 | 4209 | 7759 | None |
| Genewiz | TFORF0570 | ZNF502 | NM_001134442, NM_001134440, NM_001282880, | 660 | 4210 | 7760 | None |
| | | | NM_001134441, NM_033210, XM_017007440, | | | | |
| | | | ENST00000449836, ENST00000436624, ENST00000296091 | | | | |
| Genewiz | TFORF0571 | MEOX1 | NM_001040002, ENST00000393661 | 661 | 4211 | 7761 | None |
| Genewiz | TFORF0572 | MEOX1 | NM_013999, ENST00000329168 | 662 | 4212 | 7762 | None |
| Genewiz | TFORF0573 | MEOX2 | NM_005924, ENST00000262041 | 663 | 4213 | 7763 | None |
| Genewiz | TFORF0574 | HOXA13 | NM_000522, ENST00000222753 | 664 | 4214 | 7764 | None |
| Genewiz | TFORF0575 | HOXA11 | NM_005523, ENST00000006015 | 665 | 4215 | 7765 | None |
| Genewiz | TFORF0576 | HOXA10 | NM_018951, ENST00000283921 | 666 | 4216 | 7766 | None |
| Genewiz | TFORF0577 | GTF2A1L | NM_001193487, ENST00000430487 | 667 | 4217 | 7767 | None |
| Genewiz | TFORF0578 | ZNF107 | XM_017012284, XM_017012283, XM_017012285, | 668 | 4218 | 7768 | None |
| | | | NM_001013746, NM_016220, XM_017012286, | | | | |
| | | | ENST00000344930, ENST00000423627, ENST00000395391 | | | | |
| Genewiz | TFORF0579 | ZNF107 | NM_001282359, ENST00000620827 | 669 | 4219 | 7769 | None |
| Genewiz | TFORF0580 | ZNF107 | NM_001282360, ENST00000613690 | 670 | 4220 | 7770 | None |
| Genewiz | TFORF0581 | ZNF101 | NM_033204, ENST00000318110, ENST00000592502 | 671 | 4221 | 7771 | None |
| Genewiz | TFORF0582 | ZNF100 | NM_173531, ENST00000358296 | 672 | 4222 | 7772 | None |
| Genewiz | TFORF0583 | ZNF324 | NM_014347, ENST00000536459, ENST00000196482 | 673 | 4223 | 7773 | None |
| Genewiz | TFORF0584 | SRF | NM_003131, ENST00000265354 | 674 | 4224 | 7774 | None |
| Genewiz | TFORF0585 | ZNF329 | NM_024620, XM_006723382, XM_011527316, | 675 | 4225 | 7775 | None |
| | | | XM_006723381, XM_017027311, XM_017027310, | | | | |
| | | | XM_017027307, XM_017027308, XM_006723383, | | | | |
| | | | XM_011527315, XM_006723384, XM_017027309, | | | | |
| | | | ENST00000597186, ENST00000598312, ENST00000358067, | | | | |
| | | | ENST00000500161 | | | | |
| Genewiz | TFORF0586 | SRY | NM_003140, ENST00000383070 | 676 | 4226 | 7776 | None |
| Genewiz | TFORF0587 | NMI | XM_017005247, XM_005246941, NM_004688, | 677 | 4227 | 7777 | None |
| | | | ENST00000243346 | | | | |
| Genewiz | TFORF0588 | FOXO3 | XM_017010586, XM_011535628, XM_011535629, | 678 | 4228 | 7778 | None |
| | | | XM_005266868, ENST00000540898 | | | | |
| Genewiz | TFORF0589 | FOXO1 | NM_002015, ENST00000379561 | 679 | 4229 | 7779 | None |
| Genewiz | TFORF0590 | FOXO6 | NM_001291281 | 680 | 4230 | 7780 | None |
| Genewiz | TFORF0591 | FOXO4 | NM_005938, ENST00000374259 | 681 | 4231 | 7781 | None |
| Genewiz | TFORF0592 | FOXO4 | NM_001170931, ENST00000341558 | 682 | 4232 | 7782 | None |
| Genewiz | TFORF0593 | UNCX | NM_001080461, ENST00000316333 | 683 | 4233 | 7783 | None |
| Genewiz | TFORF0594 | ANKRD30A | NM_052997, ENST00000361713, ENST00000602533 | 684 | 4234 | 7784 | None |
| Genewiz | TFORF0595 | PLAG1 | NM_002655, NM_001114634, XM_017013576, | 685 | 4235 | 7785 | None |
| | | | ENST00000316981, ENST00000429357 | | | | |
| Genewiz | TFORF0596 | PLAG1 | NM_001114635, XM_011517544, XM_017013577, | 686 | 4236 | 7786 | None |
| | | | ENST00000423799 | | | | |
| Genewiz | TFORF0597 | SMARCB1 | NM_001317946, ENST00000344921 | 687 | 4237 | 7787 | None |
| Genewiz | TFORF0598 | SMARCB1 | NM_003073, ENST00000263121 | 688 | 4238 | 7788 | None |
| Genewiz | TFORF0599 | ZNF891 | XM_017018666, XM_017018667, XM_017018668, | 689 | 4239 | 7789 | None |
| | | | NM_001277291, ENST00000537226 | | | | |
| Genewiz | TFORF0600 | MZF1 | NM_001267033, ENST00000594234 | 690 | 4240 | 7790 | None |
| Genewiz | TFORF0601 | ZNF726 | NM_001244038, ENST00000594466 | 691 | 4241 | 7791 | None |
| Genewiz | TFORF0602 | ZNF728 | NM_001267716, ENST00000594710 | 692 | 4242 | 7792 | None |
| Genewiz | TFORF0603 | ZNF292 | NM_015021, ENST00000369577 | 693 | 4243 | 7793 | None |
| Genewiz | TFORF0604 | PUF60 | NM_001271099, ENST00000456095 | 694 | 4244 | 7794 | None |
| Genewiz | TFORF0605 | PUF60 | NM_001271097, ENST00000527197 | 695 | 4245 | 7795 | None |
| Genewiz | TFORF0606 | PUF60 | NM_078480, ENST00000526683 | 696 | 4246 | 7796 | None |
| Genewiz | TFORF0607 | CREBL2 | NM_001310, ENST00000228865 | 697 | 4247 | 7797 | None |
| Genewiz | TFORF0608 | MAFA | NM_201589, ENST00000333480 | 698 | 4248 | 7798 | None |
| Genewiz | TFORF0609 | MAFF | NM_001161574, ENST00000538999 | 699 | 4249 | 7799 | None |
| Genewiz | TFORF0610 | RBPJ | XM_005248161, XM_017008175, XM_017008174, | 700 | 4250 | 7800 | None |
| | | | NM_203284, XM_011513840, XM_017008173, | | | | |
| | | | ENST00000355476, ENST00000342320 | | | | |
| Genewiz | TFORF0611 | RBPJ | XM_017008172, NM_005349, ENST00000342295, | 701 | 4251 | 7801 | None |
| | | | ENST00000361572 | | | | |
| Genewiz | TFORF0612 | RBPJ | NM_015874, ENST00000348160 | 702 | 4252 | 7802 | None |
| Genewiz | TFORF0613 | ZSCAN29 | NM_152455, XM_006720401, XM_011521266, | 703 | 4253 | 7803 | None |
| | | | ENST00000396976 | | | | |
| Genewiz | TFORF0614 | ZSCAN25 | XM_011515906, XM_011515907, XM_011515905, | 704 | 4254 | 7804 | None |
| | | | NM_145115, XM_005250194, XM_017011824, | | | | |
| | | | ENST00000394152, ENST00000334715 | | | | |
| Genewiz | TFORF0615 | ZSCAN26 | NM_001287422, ENST00000611552 | 705 | 4255 | 7805 | None |
| Genewiz | TFORF0616 | ZSCAN26 | NM_152736, NM_001287421, ENST00000316606, | 706 | 4256 | 7806 | None |
| | | | ENST00000614088 | | | | |
| Genewiz | TFORF0617 | ZSCAN26 | NM_001023560, XM_011514862, ENST00000421553, | 707 | 4257 | 7807 | None |
| | | | ENST00000619937 | | | | |
| Genewiz | TFORF0618 | ZSCAN26 | NM_001111039, XM_017011264, ENST00000623276 | 708 | 4258 | 7808 | None |
| Genewiz | TFORF0619 | ZSCAN20 | XM_017002237, NM_145238, XM_006710874, | 709 | 4259 | 7809 | None |
| | | | ENST00000361328 | | | | |
| Genewiz | TFORF0620 | ZSCAN21 | XM_017012585, ENST00000456748 | 710 | 4260 | 7810 | None |
| Genewiz | TFORF0621 | ZSCAN22 | NM_001321116, NM_181846, XM_006723192, | 711 | 4261 | 7811 | None |
| | | | XM_011526917, ENST00000329665 | | | | |
| Genewiz | TFORF0622 | ZSCAN23 | XM_017010479, XM_011514406, XM_011514408, | 712 | 4262 | 7812 | None |
| | | | XM_011514410, XM_005248950, NM_001012455, | | | | |
| | | | ENST00000289788 | | | | |
| Genewiz | TFORF0623 | DDIT3 | NM_001195056, NM_001195053, NM_001195054, | 713 | 4263 | 7813 | None |
| | | | NM_001195055, ENST00000551116, ENST00000552740 | | | | |
| Genewiz | TFORF0624 | NKRF | NM_001173487, ENST00000542113 | 714 | 4264 | 7814 | None |
| Genewiz | TFORF0625 | MYSM1 | NM_001085487, ENST00000472487 | 715 | 4265 | 7815 | None |
| Genewiz | TFORF0626 | CEBPZ | NM_005760, ENST00000234170 | 716 | 4266 | 7816 | None |
| Genewiz | TFORF0627 | TERF2 | NM_005652, ENST00000254942 | 717 | 4267 | 7817 | None |
| Genewiz | TFORF0628 | NR112 | NM_033013, ENST00000466380, ENST00000638727 | 718 | 4268 | 7818 | None |
| Genewiz | TFORF0629 | NR112 | NM_022002, ENST00000337940 | 719 | 4269 | 7819 | None |
| Genewiz | TFORF0630 | NR112 | NM_003889, ENST00000393716, ENST00000640105 | 720 | 4270 | 7820 | None |
| Genewiz | TFORF0631 | TERF1 | NM_017489, ENST00000276603 | 721 | 4271 | 7821 | None |
| Genewiz | TFORF0632 | VPS72 | NM_005997, ENST00000368892 | 722 | 4272 | 7822 | None |
| Genewiz | TFORF0633 | VPS72 | NM_001271087, ENST00000354473 | 723 | 4273 | 7823 | None |
| Genewiz | TFORF0634 | TRIM33 | NM_033020, ENST00000369543 | 724 | 4274 | 7824 | None |
| Genewiz | TFORF0635 | TRIM33 | NM_015906, ENST00000358465 | 725 | 4275 | 7825 | None |
| Genewiz | TFORF0636 | HOXC5 | NM_018953, ENST00000312492 | 726 | 4276 | 7826 | None |
| Genewiz | TFORF0637 | HOXC4 | NM_014620, NM_153633, ENST00000303406, | 727 | 4277 | 7827 | None |
| | | | ENST00000430889 | | | | |
| Genewiz | TFORF0638 | HOXC6 | NM_153693, ENST00000394331 | 728 | 4278 | 7828 | None |
| Genewiz | TFORF0639 | HOXC6 | NM_004503, ENST00000243108 | 729 | 4279 | 7829 | None |
| Genewiz | TFORF0640 | NHP2 | NM_001034833, ENST00000314397 | 730 | 4280 | 7830 | None |
| Genewiz | TFORF0641 | THAP11 | NM_020457, ENST00000303596 | 731 | 4281 | 7831 | None |
| Genewiz | TFORF0642 | NR113 | NM_005122, ENST00000367983 | 732 | 4282 | 7832 | None |
| Genewiz | TFORF0643 | NR113 | NM_001077469, ENST00000428574 | 733 | 4283 | 7833 | None |
| Genewiz | TFORF0644 | NR113 | NM_001077471, ENST00000367984 | 734 | 4284 | 7834 | None |
| Genewiz | TFORF0645 | NR113 | NM_001077481, ENST00000367985 | 735 | 4285 | 7835 | None |
| Genewiz | TFORF0646 | NR113 | NM_001077477, ENST00000437437 | 736 | 4286 | 7836 | None |
| Genewiz | TFORF0647 | NR113 | NM_001077478, ENST00000442691 | 737 | 4287 | 7837 | None |
| Genewiz | TFORF0648 | NR113 | NM_001077475, ENST00000512372 | 738 | 4288 | 7838 | None |
| Genewiz | TFORF0649 | NR113 | NM_001077476, ENST00000508740 | 739 | 4289 | 7839 | None |
| Genewiz | TFORF0650 | NR113 | NM_001077473, ENST00000412844 | 740 | 4290 | 7840 | None |
| Genewiz | TFORF0651 | NR113 | NM_001077482, ENST00000367980, ENST00000367979 | 741 | 4291 | 7841 | None |
| Genewiz | TFORF0652 | NR113 | NM_001077470, ENST00000504010 | 742 | 4292 | 7842 | None |
| Genewiz | TFORF0653 | NR113 | NM_001077479, ENST00000511676 | 743 | 4293 | 7843 | None |
| Genewiz | TFORF0654 | NR113 | NM_001077472, ENST00000367981 | 744 | 4294 | 7844 | None |
| Genewiz | TFORF0655 | NR113 | NM_001077474, ENST00000505005 | 745 | 4295 | 7845 | None |
| Genewiz | TFORF0656 | CHD3 | NM_001005273, ENST00000330494 | 746 | 4296 | 7846 | None |
| Genewiz | TFORF0657 | CHD3 | NM_005852, ENST00000358181 | 747 | 4297 | 7847 | None |
| Genewiz | TFORF0658 | CHD3 | NM_001005271, ENST00000380358 | 748 | 4298 | 7848 | None |
| Genewiz | TFORF0659 | CHD5 | NM_015557, ENST00000262450 | 749 | 4299 | 7849 | None |
| Genewiz | TFORF0660 | CHD7 | NM_001316690, ENST00000524602 | 750 | 4300 | 7850 | None |
| Genewiz | TFORF0661 | CHD7 | NM_017780, ENST00000423902 | 751 | 4301 | 7851 | None |
| Genewiz | TFORF0662 | CHD6 | NM_032221, ENST00000373233 | 752 | 4302 | 7852 | None |
| Genewiz | TFORF0663 | MNT | NM_020310, ENST00000174618 | 753 | 4303 | 7853 | None |
| Genewiz | TFORF0664 | NKX1-2 | NM_001146340, ENST00000451024 | 754 | 4304 | 7854 | None |
| Genewiz | TFORF0665 | SCX | XM_006716616, NM_001080514, ENST00000567180 | 755 | 4305 | 7855 | None |
| Genewiz | TFORF0666 | TGFB111 | NM_001164719, NM_015927, ENST00000361773, | 756 | 4306 | 7856 | None |
| | | | ENST00000394858, ENST00000567607 | | | | |
| Genewiz | TFORF0667 | TGFB111 | NM_001042454, ENST00000394863 | 757 | 4307 | 7857 | None |
| Genewiz | TFORF0668 | ZNF823 | NM_001080493, ENST00000341191 | 758 | 4308 | 7858 | None |
| Genewiz | TFORF0669 | ZNF821 | NM_001201556, ENST00000611294 | 759 | 4309 | 7859 | None |
| Genewiz | TFORF0670 | ZNF821 | NM_017530, NM_001201554, XM_017023414, | 760 | 4310 | 7860 | None |
| | | | ENST00000313565, ENST00000446827 | | | | |
| Genewiz | TFORF0671 | ZNF821 | NM_001201553, NM_001201552, XM_017023409, | 761 | 4311 | 7861 | None |
| | | | XM_006721233, XM_017023411, XM_017023410, | | | | |
| | | | XM_011523211, XM_005256033, XM_017023412, | | | | |
| | | | ENST00000425432, ENST00000565601 | | | | |
| Genewiz | TFORF0672 | ZNF827 | NM_001306215, ENST00000508784 | 762 | 4312 | 7862 | None |
| Genewiz | TFORF0673 | ZNF827 | NM_178835, XM_017007772, XM_017007774, | 763 | 4313 | 7863 | None |
| | | | XM_017007773, XM_017007775, ENST00000379448 | | | | |
| Genewiz | TFORF0674 | ZNF829 | NM_001171979, ENST00000520965 | 764 | 4314 | 7864 | None |
| Genewiz | TFORF0675 | PRDM16 | NM_022114, ENST00000270722 | 765 | 4315 | 7865 | None |
| Genewiz | TFORF0676 | PRDM16 | XM_005244774, ENST00000511072 | 766 | 4316 | 7866 | None |
| Genewiz | TFORF0677 | PRDM16 | NM_199454, ENST00000378391 | 767 | 4317 | 7867 | None |
| Genewiz | TFORF0678 | PRDM14 | NM_024504, ENST00000276594 | 768 | 4318 | 7868 | None |
| Genewiz | TFORF0679 | PRDM15 | NM_001282934, ENST00000422911 | 769 | 4319 | 7869 | None |
| Genewiz | TFORF0680 | PRDM15 | NM_001040424, ENST00000398548 | 770 | 4320 | 7870 | None |
| Genewiz | TFORF0681 | PRDM15 | NM_022115, ENST00000433067, ENST00000269844 | 771 | 4321 | 7871 | None |
| Genewiz | TFORF0682 | PRDM15 | XM_017028426, XM_017028425, ENST00000447016, | 772 | 4322 | 7872 | None |
| | | | ENST00000447207 | | | | |
| Genewiz | TFORF0683 | PRDM12 | NM_021619, ENST00000253008 | 773 | 4323 | 7873 | None |
| Genewiz | TFORF0684 | PRDM13 | NM_021620, ENST00000369215 | 774 | 4324 | 7874 | None |
| Genewiz | TFORF0685 | PRDM10 | NM_020228, ENST00000358825 | 775 | 4325 | 7875 | None |
| Genewiz | TFORF0686 | PRDM10 | NM_199438, ENST00000423662 | 776 | 4326 | 7876 | None |
| Genewiz | TFORF0687 | PRDM10 | NM_199437, ENST00000360871 | 777 | 4327 | 7877 | None |
| Genewiz | TFORF0688 | PRDM10 | NM_199439, ENST00000304538 | 778 | 4328 | 7878 | None |
| Genewiz | TFORF0689 | HNF4G | XM_017013374, XM_011517516, XM_017013373, | 779 | 4329 | 7879 | None |
| | | | XM_017013375, XM_017013376, ENST00000354370 | | | | |
| Genewiz | TFORF0690 | HNF4G | NM_004133, ENST00000396423 | 780 | 4330 | 7880 | None |
| Genewiz | TFORF0691 | HNF4A | NM_178849, ENST00000415691 | 781 | 4331 | 7881 | None |
| Genewiz | TFORF0692 | HNF4A | NM_001030003, ENST00000457232 | 782 | 4332 | 7882 | None |
| Genewiz | TFORF0693 | HNF4A | NM_001287183, ENST00000619550 | 783 | 4333 | 7883 | None |
| Genewiz | TFORF0694 | HNF4A | NM_175914, ENST00000316673 | 784 | 4334 | 7884 | None |
| Genewiz | TFORF0695 | HNF4A | NM_001030004, ENST00000609795 | 785 | 4335 | 7885 | None |
| Genewiz | TFORF0696 | GTF3A | NM_002097, ENST00000381140, ENST00000640289 | 786 | 4336 | 7886 | None |
| Genewiz | TFORF0697 | BATF2 | NM_138456, ENST00000301887 | 787 | 4337 | 7887 | None |
| Genewiz | TFORF0698 | BATF2 | NM_001300808, ENST00000435842 | 788 | 4338 | 7888 | None |
| Genewiz | TFORF0699 | BATF2 | NM_001300807, ENST00000527716 | 789 | 4339 | 7889 | None |
| Genewiz | TFORF0700 | PAXBP1 | NM_013329, ENST00000290178 | 790 | 4340 | 7890 | None |
| Genewiz | TFORF0701 | PAXBP1 | NM_016631, ENST00000331923 | 791 | 4341 | 7891 | None |
| Genewiz | TFORF0702 | NCOR2 | NM_006312, ENST00000405201 | 792 | 4342 | 7892 | None |
| Genewiz | TFORF0703 | NCOR2 | NM_001077261, ENST00000404621 | 793 | 4343 | 7893 | None |
| Genewiz | TFORF0704 | NCOR2 | NM_001206654, ENST00000429285 | 794 | 4344 | 7894 | None |
| Genewiz | TFORF0705 | NCOR1 | NM_006311, ENST00000268712 | 795 | 4345 | 7895 | None |
| Genewiz | TFORF0706 | NCOR1 | NM_001190438, ENST00000395848 | 796 | 4346 | 7896 | None |
| Genewiz | TFORF0707 | NCOR1 | NM_001190440, XM_011524086, ENST00000395851 | 797 | 4347 | 7897 | None |
| Genewiz | TFORF0708 | ZNF628 | NM_033113, ENST00000598519 | 798 | 4348 | 7898 | None |
| Genewiz | TFORF0709 | ZNF629 | NM_001080417, ENST00000262525 | 799 | 4349 | 7899 | None |
| Genewiz | TFORF0710 | ZNF624 | NM_020787, XM_006721562, ENST00000311331 | 800 | 4350 | 7900 | None |
| Genewiz | TFORF0711 | ZNF625 | NM_145233, ENST00000439556 | 801 | 4351 | 7901 | None |
| Genewiz | TFORF0712 | ZNF626 | NM_001076675, ENST00000601440 | 802 | 4352 | 7902 | None |
| Genewiz | TFORF0713 | ZNF627 | NM_145295, ENST00000361113 | 803 | 4353 | 7903 | None |
| Genewiz | TFORF0714 | ZNF620 | XM_017006069, NM_001256167, NM_001256168, | 804 | 4354 | 7904 | None |
| | | | ENST00000418905 | | | | |
| Genewiz | TFORF0715 | ZNF620 | XM_005265012, XM_005265011, NM_175888, | 805 | 4355 | 7905 | None |
| | | | ENST00000314529 | | | | |
| Genewiz | TFORF0716 | ZNF621 | NM_001287245, ENST00000310898 | 806 | 4356 | 7906 | None |
| Genewiz | TFORF0717 | TER | NM_001145398, ENST00000406644 | 807 | 4357 | 7907 | None |
| Genewiz | TFORF0718 | TEF | NM_003216, ENST00000266304 | 808 | 4358 | 7908 | None |
| Genewiz | TFORF0719 | ZNF623 | NM_001261843, NM_001082480, XM_006716708, | 809 | 4359 | 7909 | None |
| | | | ENST00000526926, ENST00000458270 | | | | |
| Genewiz | TFORF0720 | ZNF623 | NM_014789, ENST00000501748 | 810 | 4360 | 7910 | None |
| Genewiz | TFORF0721 | ZNF727 | NM_001159522, ENST00000456806 | 811 | 4361 | 7911 | None |
| Genewiz | TFORF0722 | NFKB1 | NM_001165412, NM_001319226, ENST00000394820, | 812 | 4362 | 7912 | None |
| | | | ENST00000505458 | | | | |
| Genewiz | TFORF0723 | NFKB1 | NM_003998, ENST00000226574 | 813 | 4363 | 7913 | None |
| Genewiz | TFORF0724 | NFKB2 | NM_001288724, NM_002502, NM_001261403, | 814 | 4364 | 7914 | None |
| | | | ENST00000428099, ENST00000189444 | | | | |
| Genewiz | TFORF0725 | NFKB2 | NM_001077494, NM_001322934, ENST00000369966 | 815 | 4365 | 7915 | None |
| Genewiz | TFORF0726 | ASH1L | NM_018489, XM_006711451, XM_006711450, | 816 | 4366 | 7916 | None |
| | | | XM_017001784, XM_017001785, ENST00000392403 | | | | |
| Genewiz | TFORF0727 | ZFP36L2 | NM_006887, ENST00000282388 | 817 | 4367 | 7917 | None |
| Genewiz | TFORF0728 | MLXIP | NM_014938, ENST00000319080 | 818 | 4368 | 7918 | None |
| Genewiz | TFORF0729 | ZNF518B | NM_053042, XM_017008786, XM_017008784, | 819 | 4369 | 7919 | None |
| | | | XM_017008785, XM_005248193, ENST00000326756 | | | | |
| Genewiz | TFORF0730 | ZNF518A | XM_017016994, XM_011540413, NM_001278524, | 820 | 4370 | 7920 | None |
| | | | NM_014803, XM_011540415, XM_011540412, | | | | |
| | | | XM_011540419, XM_017016993, XM_011540406, | | | | |
| | | | XM_011540410, XM_011540418, XM_011540420, | | | | |
| | | | XM_017016989, XM_011540408, NM_001278525, | | | | |
| | | | XM_017016992, XM_017016991, XM_017016986, | | | | |
| | | | XM_017016995, XM_017016990, XM_017016987, | | | | |
| | | | XM_017016996, XM_017016988, XM_017016997, | | | | |
| | | | XM_017016999, XM_017016998, ENST00000316045, | | | | |
| | | | ENST00000624776, ENST00000614149 | | | | |
| Genewiz | TFORF0731 | PROX1 | NM_001270616, NM_002763, XM_017001833, | 821 | 4371 | 7921 | None |
| | | | ENST00000498508, ENST00000366958, ENST00000261454, | | | | |
| | | | ENST00000435016 | | | | |
| Genewiz | TFORF0732 | PROX2 | NM_001243007, ENST00000556489 | 822 | 4372 | 7922 | None |
| Genewiz | TFORF0733 | PROX2 | NM_001080408, ENST00000556084 | 823 | 4373 | 7923 | None |
| Genewiz | TFORF0734 | ZNF488 | XM_017015642, XM_017015643, XM_011539244, | 824 | 4374 | 7924 | None |
| | | | XM_006717617, NM_153034, ENST00000585316 | | | | |
| Genewiz | TFORF0735 | ZNF480 | NM_001297625, ENST00000335090 | 825 | 4375 | 7925 | None |
| Genewiz | TFORF0736 | ZNF480 | NM_001297624, ENST00000334564 | 826 | 4376 | 7926 | None |
| Genewiz | TFORF0737 | ZNF480 | NM_144684, XM_011526465, ENST00000468240, | 827 | 4377 | 7927 | None |
| | | | ENST00000595962 | | | | |
| Genewiz | TFORF0738 | ZNF155 | XM_005259215, NM_001260487, NM_198089, | 828 | 4378 | 7928 | None |
| | | | NM_003445, NM_001260486, ENST00000611002, | | | | |
| | | | ENST00000270014, ENST00000590615 | | | | |
| Genewiz | TFORF0739 | ZNF155 | XM_011527279, NM_001260488, XM_017027248, | 829 | 4379 | 7929 | None |
| | | | XM_011527278, ENST00000407951 | | | | |
| Genewiz | TFORF0740 | JDP2 | XM_017020975, XM_017020973, NM_001135047, | 830 | 4380 | 7930 | None |
| | | | XM_017020974, NM_001135048, NM_130469, | | | | |
| | | | XM_005267332, ENST00000419727, ENST0437176, | | | | |
| | | | ENST00000435893 | | | | |
| Genewiz | TFORF0741 | JDP2 | XM_017020972, NM_001135049, ENST00000267569 | 831 | 4381 | 7931 | None |
| Genewiz | TFORF0742 | ZNF485 | NM_001318141, NM_145312, NM_001318140, | 832 | 4382 | 7932 | None |
| | | | ENST00000361807, ENST00000374435 | | | | |
| Genewiz | TFORF0743 | ZNF486 | NM_052852, ENST00000335117 | 833 | 4383 | 7933 | None |
| Genewiz | TFORF0744 | PIAS2 | NM_004671, ENST00000585916 | 834 | 4384 | 7934 | None |
| Genewiz | TFORF0745 | PIAS2 | NM_173206, ENST00000324794 | 835 | 4385 | 7935 | None |
| Genewiz | TFORF0746 | PIAS3 | NM_006099, ENST00000393045 | 836 | 4386 | 7936 | None |
| Genewiz | TFORF0747 | PIAS1 | NM_001320687, XM_017022688, ENST00000545237 | 837 | 4387 | 7937 | None |
| Genewiz | TFORF0748 | PIAS4 | NM_015897, ENST00000262971 | 838 | 4388 | 7938 | None |
| Genewiz | TFORF0749 | ZNF131 | XM_005248359, XM_005248362, NM_001297548, | 839 | 4389 | 7939 | None |
| | | | XM_005248360, XM_017009830, XM_005248361, | | | | |
| | | | XM_005248363, ENST00000509156 | | | | |
| Genewiz | TFORF0750 | ZNF131 | XM_005248365, XM_017009834, NM_003432, | 840 | 4390 | 7940 | None |
| | | | XM_017009835, XM_017009832, XM_017009833, | | | | |
| | | | ENST00000306938, ENST00000505606, ENST00000509634 | | | | |
| Genewiz | TFORF0751 | HIVEP1 | XM_011514551, XM_011514552, NM_002114, | 841 | 4391 | 7941 | None |
| | | | XM_011514553, ENST00000379388 | | | | |
| Genewiz | TFORF0752 | HIVEP3 | NM_001127714, XM_017001994, ENST00000372584 | 842 | 4392 | 7942 | None |
| Genewiz | TFORF0753 | HIVEP3 | XM_011541884, NM_024503, XM_017001993, | 843 | 4393 | 7943 | None |
| | | | XM_017001992, ENST00000372583 | | | | |
| Genewiz | TFORF0754 | ZNF317 | NM_001190791, ENST00000360385 | 844 | 4394 | 7944 | None |
| Genewiz | TFORF0755 | ZNF319 | NM_020807, XM_005256069, ENST00000299237 | 845 | 4395 | 7945 | None |
| Genewiz | TFORF0756 | JUND | NM_005354, ENST00000252818 | 846 | 4396 | 7946 | None |
| Genewiz | TFORF0757 | NOTCH1 | NM_017617, ENST00000277541 | 847 | 4397 | 7947 | None |
| Genewiz | TFORF0758 | OVOL3 | NM_001302757, ENST00000633214 | 848 | 4398 | 7948 | None |
| Genewiz | TFORF0759 | TLX2 | NM_016170, ENST00000233638 | 849 | 4399 | 7949 | None |
| Genewiz | TFORF0760 | OVOL1 | XM_011545067, XM_017017837, XM_005274018, | 850 | 4400 | 7950 | None |
| | | | XM_011545068, XM_017017838, ENST00000532448 | | | | |
| Genewiz | TFORF0761 | ZNF556 | NM_024967, ENST00000307635 | 851 | 4401 | 7951 | None |
| Genewiz | TFORF0762 | ZNF557 | NM_001044388, ENST00000414706 | 852 | 4402 | 7952 | None |
| Genewiz | TFORF0763 | ZNF557 | NM_001044387, NM_024341, ENST00000252840 | 853 | 4403 | 7953 | None |
| Genewiz | TFORF0764 | ZNF555 | NM_001172775, ENST00000591539 | 854 | 4404 | 7954 | None |
| Genewiz | TFORF0765 | ZNF555 | NM_152791, ENST00000334241 | 855 | 4405 | 7955 | None |
| Genewiz | TFORF0766 | ZNF552 | NM_024762, ENST00000391701 | 856 | 4406 | 7956 | None |
| Genewiz | TFORF0767 | ZNF550 | NM_001277090, NM_001277091, NM_001277092, | 857 | 4407 | 7957 | None |
| | | | XM_011526567, XM_017026401, ENST00000457177, | | | | |
| | | | ENST00000325134, ENST00000376230, ENST00000447310 | | | | |
| Genewiz | TFORF0768 | ZNF551 | NM_138347, ENST00000282296 | 858 | 4408 | 7958 | None |
| Genewiz | TFORF0769 | MSX1 | NM_002448, ENST00000382723 | 859 | 4409 | 7959 | None |
| Genewiz | TFORF0770 | MSX2 | NM_002449, ENST00000239243 | 860 | 4410 | 7960 | None |
| Genewiz | TFORF0771 | MSX2 | XM_017009489, ENST00000507785 | 861 | 4411 | 7961 | None |
| Genewiz | TFORF0772 | SIN3A | NM_015477, NM_001145358, NM_001145357, | 862 | 4412 | 7962 | None |
| | | | XM_006720466, XM_006720467, XM_006720465, | | | | |
| | | | ENST00000394947, ENST00000360439, ENST00000394949 | | | | |
| Genewiz | TFORF0773 | ZNF92 | NM_001287532, ENST00000357512 | 863 | 4413 | 7963 | None |
| Genewiz | TFORF0774 | ZNF92 | NM_001287534, NM_001287533, ENST00000431504 | 864 | 4414 | 7964 | None |
| Genewiz | TFORF0775 | ZNF92 | NM_007139, ENST00000450302 | 865 | 4415 | 7965 | None |
| Genewiz | TFORF0776 | ZNF92 | NM_152626, ENST00000328747 | 866 | 4416 | 7966 | None |
| Genewiz | TFORF0777 | SHOX | NM_000451, NM_000451, ENST00000381578, | 867 | 4417 | 7967 | None |
| | | | ENST00000554971, ENST00000381578, ENST00000554971 | | | | |
| Genewiz | TFORF0778 | SHOX | NM_006883, NM_006883, ENST00000334060, | 868 | 4418 | 7968 | None |
| | | | ENST00000381575, ENST00000334060, ENST00000381575 | | | | |
| Genewiz | TFORF0779 | GTF2H2 | NM_001515, XM_017009404, XM_017009405, | 869 | 4419 | 7969 | None |
| | | | XM_017009406, XM_017009403, ENST00000274400, | | | | |
| | | | ENST00000330280 | | | | |
| Genewiz | TFORF0780 | GTF2H1 | NM_005316, NM_001142307, XM_006718208, | 870 | 4420 | 7970 | None |
| | | | ENST00000453096, ENST00000265963 | | | | |
| Genewiz | TFORF0781 | ZNF93 | NM_031218, ENST00000343769 | 871 | 4421 | 7971 | None |
| Genewiz | TFORF0782 | FOXN4 | NM_213596, ENST00000299162 | 872 | 4422 | 7972 | None |
| Genewiz | TFORF0783 | SEBOX | NM_001080837, ENST00000536498 | 873 | 4423 | 7973 | None |
| Genewiz | TFORF0784 | ZNF585A | NM_001288800, ENST00000292841 | 874 | 4424 | 7974 | None |
| Genewiz | TFORF0785 | ZNF585A | NM_199126, NM_152655, ENST00000356958, | 875 | 4425 | 7975 | None |
| | | | ENST00000392157 | | | | |
| Genewiz | TFORF0786 | FOXN1 | XM_005258046, NM_003593, ENST00000579795, | 876 | 4426 | 7976 | None |
| | | | ENST00000226247 | | | | |
| Genewiz | TFORF0787 | REPIN1 | XM_006715949, NM_001099695, XM_006715947, | 877 | 4427 | 7977 | None |
| | | | XM_006715948, ENST00000489432 | | | | |
| Genewiz | TFORF0788 | REPIN1 | XM_005249985, XM_006715952, NM_001099696, | 878 | 4428 | 7978 | None |
| | | | NM_013400, XM_006715953, XM_017012082, | | | | |
| | | | XM_017012081, XM_011516112, NM_014374, | | | | |
| | | | ENST00000397281, ENST00000444957, ENST00000425389 | | | | |
| Genewiz | TFORF0789 | DBX2 | NM_001004329, ENST00000332700 | 879 | 4429 | 7979 | None |
| Genewiz | TFORF0790 | DBX1 | NM_001029865, ENST00000524983 | 880 | 4430 | 7980 | None |
| Genewiz | TFORF0791 | ZNF99 | NM_001080409, ENST00000596209 | 881 | 4431 | 7981 | None |
| Genewiz | TFORF0792 | TARBP2 | NM_004178, XM_005269114, XM_005269115, | 882 | 4432 | 7982 | None |
| | | | NM_134324, ENST0456234, ENST00000394357 | | | | |
| Genewiz | TFORF0793 | ATF5 | NM_012068, NM_001193646, XM_011526629, | 883 | 4433 | 7983 | None |
| | | | NM_001290746, ENST00000595125, ENST00000423777 | | | | |
| Genewiz | TFORF0794 | ATF7 | XM_005268587, XM_017018722, NM_006856, | 884 | 4434 | 7984 | None |
| | | | ENST00000420353, ENST00000456903 | | | | |
| Genewiz | TFORF0795 | ATF7 | NM_001206683, NM_001206682, ENST00000548118, | 885 | 4435 | 7985 | None |
| | | | ENST00000591397 | | | | |
| Genewiz | TFORF0796 | ATF6 | NM_007348, ENST00000367942 | 886 | 4436 | 7986 | None |
| Genewiz | TFORF0797 | ATF1 | XM_011538387, NM_005171, XM_017019334, | 887 | 4437 | 7987 | None |
| | | | XM_017019333, XM_011538386, XM_017019332, | | | | |
| | | | ENST00000262053 | | | | |
| Genewiz | TFORF0798 | ATF3 | NM_001206486, ENST00000336937 | 888 | 4438 | 7988 | None |
| Genewiz | TFORF0799 | ATF3 | NM_001040619, ENST00000366983, ENST00000464547 | 889 | 4439 | 7989 | None |
| Genewiz | TFORF0800 | ATF3 | NM_001206488, NM_001206484, ENST00000613954, | 890 | 4440 | 7990 | None |
| | | | ENST00000613104 | | | | |
| Genewiz | TFORF0801 | ATF3 | NM_001030287, XM_011509579, NM_001674, | 891 | 4441 | 7991 | None |
| | | | XM_005273146, ENST00000366987, ENST00000341491 | | | | |
| Genewiz | TFORF0802 | ATF2 | NM_001256091, ENST00000426833 | 892 | 4442 | 7992 | None |
| Genewiz | TFORF0803 | ATF2 | NM_001256092, ENST00000345739, ENST00000409635 | 893 | 4443 | 7993 | None |
| Genewiz | TFORF0804 | SMARCC2 | XM_005269101, ENST00000550164 | 894 | 4444 | 7994 | None |
| Genewiz | TFORF0805 | SMARCC2 | NM_001130420, ENST00000394023 | 895 | 4445 | 7995 | None |
| Genewiz | TFORF0806 | SMARCC2 | NM_139067, ENST00000347471 | 896 | 4446 | 7996 | None |
| Genewiz | TFORF0807 | SMARCC2 | NM_003075, ENST00000267064 | 897 | 4447 | 7997 | None |
| Genewiz | TFORF0808 | SMARCC1 | NM_003074, ENST00000254480 | 898 | 4448 | 7998 | None |
| Genewiz | TFORF0809 | SOHLH1 | NM_001012415, ENST00000298466 | 899 | 4449 | 7999 | None |
| Genewiz | TFORF0810 | SOHLH1 | NM_001101677, ENST00000425225 | 900 | 4450 | 8000 | None |
| Genewiz | TFORF0811 | CDCA7L | NM_018719, ENST00000406877 | 901 | 4451 | 8001 | None |
| Genewiz | TFORF0812 | CDCA7L | NM_001127370, ENST00000356195 | 902 | 4452 | 8002 | None |
| Genewiz | TFORF0813 | CDCA7L | NM_001127371, ENST00000373934 | 903 | 4453 | 8003 | None |
| Genewiz | TFORF0814 | LHX1 | NM_005568, ENST00000614239 | 904 | 4454 | 8004 | None |
| Genewiz | TFORF0815 | LHX2 | NM_004789, ENST00000373615 | 905 | 4455 | 8005 | None |
| Genewiz | TFORF0816 | LHX3 | NM_014564, ENST00000371746 | 906 | 4456 | 8006 | None |
| Genewiz | TFORF0817 | LHX3 | NM_178138, ENST00000371748 | 907 | 4457 | 8007 | None |
| Genewiz | TFORF0818 | LHX3 | XM_005263410, ENST00000619587 | 908 | 4458 | 8008 | None |
| Genewiz | TFORF0819 | LHX5 | NM_022363, ENST00000261731 | 909 | 4459 | 8009 | None |
| Genewiz | TFORF0820 | LHX6 | NM_014368, ENST00000394319 | 910 | 4460 | 8010 | None |
| Genewiz | TFORF0821 | LHX6 | NM_001242333, ENST00000541397 | 911 | 4461 | 8011 | None |
| Genewiz | TFORF0822 | LHX6 | NM_199160, ENST00000340587 | 912 | 4462 | 8012 | None |
| Genewiz | TFORF0823 | LHX6 | NM_001242335, ENST00000559895 | 913 | 4463 | 8013 | None |
| Genewiz | TFORF0824 | LHX8 | NM_001256114, ENST00000356261 | 914 | 4464 | 8014 | None |
| Genewiz | TFORF0825 | LHX8 | NM_001001933, ENST00000294638 | 915 | 4465 | 8015 | None |
| Genewiz | TFORF0826 | LHX9 | XM_005245350, ENST00000561173 | 916 | 4466 | 8016 | None |
| Genewiz | TFORF0827 | LHX9 | NM_001014434, ENST00000367390 | 917 | 4467 | 8017 | None |
| Genewiz | TFORF0828 | ZNF585B | NM_152279, ENST00000532828 | 918 | 4468 | 8018 | None |
| Genewiz | TFORF0829 | SLC45A2 | NM_016180, ENST00000296589 | 919 | 4469 | 8019 | None |
| Genewiz | TFORF0830 | SLC45A2 | NM_001297417, ENST00000509381 | 920 | 4470 | 8020 | None |
| Genewiz | TFORF0831 | ZBTB33 | NM_006777, NM_001184742, ENST00000326624, | 921 | 4471 | 8021 | None |
| | | | ENST00000557385 | | | | |
| Genewiz | TFORF0832 | GTF2F1 | NM_002096, ENST00000394456 | 922 | 4472 | 8022 | None |
| Genewiz | TFORF0833 | GTF2F2 | NM_004128, ENST00000340473 | 923 | 4473 | 8023 | None |
| Genewiz | TFORF0834 | HSFY1 | NM_152584, ENST00000309834 | 924 | 4474 | 8024 | None |
| Genewiz | TFORF0835 | HSFY1 | NM_033108, ENST00000307393 | 925 | 4475 | 8025 | None |
| Genewiz | TFORF0836 | ZSCAN18 | XM_005259174, XM_011527238, XM_006723335, | 926 | 4476 | 8026 | None |
| | | | XM_011527239, NM_023926, NM_001145543, | | | | |
| | | | ENST00000240727, ENST00000601144 | | | | |
| Genewiz | TFORF0837 | ZSCAN18 | NM_001145544, ENST00000421612 | 927 | 4477 | 8027 | None |
| Genewiz | TFORF0838 | ZSCAN18 | NM_001145542, ENST00000600404 | 928 | 4478 | 8028 | None |
| Genewiz | TFORF0839 | HSFY2 | NM_001001877, ENST00000344884 | 929 | 4479 | 8029 | None |
| Genewiz | TFORF0840 | HSFY2 | NM_153716, ENST00000304790 | 930 | 4480 | 8030 | None |
| Genewiz | TFORF0841 | ZSCAN10 | NM_032805, XM_017023791, ENST00000576985 | 931 | 4481 | 8031 | None |
| Genewiz | TFORF0842 | ZSCAN10 | NM_001282415, ENST00000575108 | 932 | 4482 | 8032 | None |
| Genewiz | TFORF0843 | ZSCAN10 | NM_001282416, ENST00000538082 | 933 | 4483 | 8033 | None |
| Genewiz | TFORF0844 | ZSCAN12 | XM_011515014, XM_017011528, XM_011515015, | 934 | 4484 | 8034 | None |
| | | | NM_001163391 | | | | |
| Genewiz | TFORF0845 | ZSCAN12 | XM_011515017 | 935 | 4485 | 8035 | None |
| Genewiz | TFORF0846 | YBX1 | NM_004559, ENST00000321358 | 936 | 4486 | 8036 | None |
| Genewiz | TFORF0847 | YBX3 | NM_001145426, ENST00000279550 | 937 | 4487 | 8037 | None |
| Genewiz | TFORF0848 | YBX3 | NM_003651, ENST00000228251 | 938 | 4488 | 8038 | None |
| Genewiz | TFORF0849 | ZIC4 | NM_001168379, ENST00000425731 | 939 | 4489 | 8039 | None |
| Genewiz | TFORF0850 | ZIC4 | NM_032153, ENST00000383075, ENST00000484399, | 940 | 4490 | 8040 | None |
| | | | ENST00000473123 | | | | |
| Genewiz | TFORF0851 | ZIC4 | NM_001168378, ENST00000525172 | 941 | 4491 | 8041 | None |
| Genewiz | TFORF0852 | ZIC4 | NM_001243256, ENST00000491672 | 942 | 4492 | 8042 | None |
| Genewiz | TFORF0853 | ZIC5 | NM_033132, ENST00000267294 | 943 | 4493 | 8043 | None |
| Genewiz | TFORF0854 | VSX1 | NM_001256272, ENST00000429762 | 944 | 4494 | 8044 | None |
| Genewiz | TFORF0855 | VSX1 | XM_017027837, ENST00000409285 | 945 | 4495 | 8045 | None |
| Genewiz | TFORF0856 | VSX1 | NM_199425, ENST00000376707 | 946 | 4496 | 8046 | None |
| Genewiz | TFORF0857 | VSX1 | XM_017027838, ENST00000409958 | 947 | 4497 | 8047 | None |
| Genewiz | TFORF0858 | VSX1 | NM_001256271, ENST00000444511 | 948 | 4498 | 8048 | None |
| Genewiz | TFORF0859 | ZIC3 | XM_017029802, ENST00000370606 | 949 | 4499 | 8049 | None |
| Genewiz | TFORF0860 | NR1H4 | NM_001206978, ENST00000549996 | 950 | 4500 | 8050 | None |
| Genewiz | TFORF0861 | NR1H4 | NM_001206993, ENST00000551379 | 951 | 4501 | 8051 | None |
| Genewiz | TFORF0862 | NR1H4 | NM_001206992, ENST00000188403 | 952 | 4502 | 8052 | None |
| Genewiz | TFORF0863 | GTF21 | NM_032999, ENST00000573035 | 953 | 4503 | 8053 | None |
| Genewiz | TFORF0864 | GTF21 | NM_033001, ENST00000621734 | 954 | 4504 | 8054 | None |
| Genewiz | TFORF0865 | GTF2 | NM_033000, ENST00000614986 | 955 | 4505 | 8055 | None |
| Genewiz | TFORF0866 | GTF21 | NM_001518, ENST00000620879 | 956 | 4506 | 8056 | None |
| Genewiz | TFORF0867 | NR1H2 | NM_001256647, ENST00000411902 | 957 | 4507 | 8057 | None |
| Genewiz | TFORF0868 | NR1H3 | NM_001251934, NM_001251935, ENST00000616973 | 958 | 4508 | 8058 | None |
| Genewiz | TFORF0869 | NR1H3 | XM_011519805, XM_006718113, XM_005252706, | 959 | 4509 | 8059 | None |
| | | | XM_005252705, XM_006718112, XM_005252707, | | | | |
| | | | NM_005693, ENST00000441012, ENST00000467728 | | | | |
| Genewiz | TFORF0870 | NR1H3 | XM_005252713, NM_001130101, ENST00000407404, | 960 | 4510 | 8060 | None |
| | | | ENST00000405853 | | | | |
| Genewiz | TFORF0871 | NR1H3 | XM_011519806, ENST00000405576 | 961 | 4511 | 8061 | None |
| Genewiz | TFORF0872 | HOXB8 | XM_005257286, ENST00000576562 | 962 | 4512 | 8062 | None |
| Genewiz | TFORF0873 | HOXB8 | NM_024016, XM_017024564, ENST00000239144 | 963 | 4513 | 8063 | None |
| Genewiz | TFORF0874 | HOXB9 | NM_024017, ENST00000311177 | 964 | 4514 | 8064 | None |
| Genewiz | TFORF0875 | GLMP | NM_001256609, ENST00000614643 | 965 | 4515 | 8065 | None |
| Genewiz | TFORF0876 | GLMP | NM_001256608, ENST00000612353 | 966 | 4516 | 8066 | None |
| Genewiz | TFORF0877 | GLMP | NM_001256605, ENST00000622703 | 967 | 4517 | 8067 | None |
| Genewiz | TFORF0878 | HOXB2 | NM_002145, ENST00000330070 | 968 | 4518 | 8068 | None |
| Genewiz | TFORF0879 | HOXB3 | NM_002146, XM_011524719, XM_011524720, | 969 | 4519 | 8069 | None |
| | | | XM_011524710, XM_006721854, XM_005257277, | | | | |
| | | | ENST00000470495, ENST00000311626, ENST00000498678, | | | | |
| | | | ENST00000476342 | | | | |
| Genewiz | TFORF0880 | HOXB3 | XM_011524726, XM_005257280, ENST00000472863, | 970 | 4520 | 8070 | None |
| | | | ENST00000489475 | | | | |
| Genewiz | TFORF0881 | HOXB3 | XM_005257282, ENST00000460160 | 971 | 4521 | 8071 | None |
| Genewiz | TFORF0882 | HOXB1 | NM_002144, ENST00000239174 | 972 | 4522 | 8072 | None |
| Genewiz | TFORF0883 | HOXB7 | NM_004502, ENST00000239165 | 973 | 4523 | 8073 | None |
| Genewiz | TFORF0884 | HOXB4 | NM_024015, ENST00000332503 | 974 | 4524 | 8074 | None |
| Genewiz | TFORF0885 | PPARG | XM_011533844, ENST00000397000 | 975 | 4525 | 8075 | None |
| Genewiz | TFORF0886 | CUX2 | NM_015267, ENST00000261726 | 976 | 4526 | 8076 | None |
| Genewiz | TFORF0887 | ZNF384 | NM_001039920, ENST00000355772 | 977 | 4527 | 8077 | None |
| Genewiz | TFORF0888 | ZNF384 | NM_001135734, XM_017018942, XM_017018943, | 978 | 4528 | 8078 | None |
| | | | XM_017018941, ENST00000396801, ENST00000361959 | | | | |
| Genewiz | TFORF0889 | ZNF384 | NM_133476, XM_017018950, XM_017018949, | 979 | 4529 | 8079 | None |
| | | | ENST00000319770 | | | | |
| Genewiz | TFORF0890 | ING4 | NM_001127585, ENST00000444704 | 980 | 4530 | 8080 | None |
| Genewiz | TFORF0891 | ING4 | NM_001127582, ENST00000396807 | 981 | 4531 | 8081 | None |
| Genewiz | TFORF0892 | ING4 | NM_001127584, ENST00000446105 | 982 | 4532 | 8082 | None |
| Genewiz | TFORF0893 | ING4 | NM_016162, ENST00000341550 | 983 | 4533 | 8083 | None |
| Genewiz | TFORF0894 | ING4 | NM_001127586, ENST00000423703 | 984 | 4534 | 8084 | None |
| Genewiz | TFORF0895 | ING4 | NM_001127583, ENST00000412586 | 985 | 4535 | 8085 | None |
| Genewiz | TFORF0896 | ZNF383 | XM_017026423, XM_011526587, XM_017026422, | 986 | 4536 | 8086 | None |
| | | | XM_005258585, XM_005258587, XM_005258588, | | | | |
| | | | XM_011526586, XM_011526590, NM_152604, | | | | |
| | | | XM_017026424, XM_011526588, XM_011526589, | | | | |
| | | | ENST00000590503, ENST00000589413, ENST00000352998 | | | | |
| Genewiz | TFORF0897 | ING1 | NM_198217, ENST00000338450 | 987 | 4537 | 8087 | None |
| Genewiz | TFORF0898 | ING1 | NM_005537, ENST00000375774 | 988 | 4538 | 8088 | None |
| Genewiz | TFORF0899 | ING1 | NM_198218, ENST00000375775 | 989 | 4539 | 8089 | None |
| Genewiz | TFORF0900 | ZNF382 | NM_001256838, ENST00000439428 | 990 | 4540 | 8090 | None |
| Genewiz | TFORF0901 | ZNF382 | NM_032825, ENST00000292928 | 991 | 4541 | 8091 | None |
| Genewiz | TFORF0902 | PTF1A | NM_178161, ENST00000376504 | 992 | 4542 | 8092 | None |
| Genewiz | TFORF0903 | SPDEF | NM_001252294, ENST00000544425 | 993 | 4543 | 8093 | None |
| Genewiz | TFORF0904 | DNAJC1 | NM_022365, ENST00000376980 | 994 | 4544 | 8094 | None |
| Genewiz | TFORF0905 | DNAJC2 | NM_014377, ENST00000379263 | 995 | 4545 | 8095 | None |
| Genewiz | TFORF0906 | DNAJC2 | NM_001129887, ENST00000249270 | 996 | 4546 | 8096 | None |
| Genewiz | TFORF0907 | BCLAF1 | NM_001077441, ENST00000530767 | 997 | 4547 | 8097 | None |
| Genewiz | TFORF0908 | BCLAF1 | NM_001077440, ENST00000353331, ENST00000392348 | 998 | 4548 | 8098 | None |
| Genewiz | TFORF0909 | BCLAF1 | NM_001301038, ENST00000527759 | 999 | 4549 | 8099 | None |
| Genewiz | TFORF0910 | BCLAF1 | NM_014739, ENST00000531224 | 1000 | 4550 | 8100 | None |
| Genewiz | TFORF0911 | BCLAF1 | XM_005267237, ENST00000527536 | 1001 | 4551 | 8101 | None |
| Genewiz | TFORF0912 | ZNF831 | XM_006723698, XM_017027643, XM_017027644, | 1002 | 4552 | 8102 | None |
| | | | XM_011528534, XM_017027642, XM_005260272, | | | | |
| | | | XM_011528537, XM_011528536, XM_005260273, | | | | |
| | | | XM_011528538, NM_178457, ENST00000637017, | | | | |
| | | | ENST00000371030 | | | | |
| Genewiz | TFORF0913 | ZNF835 | NM_001005850, XM_005259383, XM_005259382, | 1003 | 4553 | 8103 | None |
| | | | ENST00000537055 | | | | |
| Genewiz | TFORF0914 | ZNF836 | XM_011526558, XM_011526559, NM_001102657, | 1004 | 4554 | 8104 | None |
| | | | ENST00000597252 | | | | |
| Genewiz | TFORF0915 | NCOA2 | NM_001321707, NM_006540, NM_001321703, | 1005 | 4555 | 8105 | None |
| | | | ENST00000452400 | | | | |
| Genewiz | TFORF0916 | NCOA3 | NM_001174088, ENST00000371997 | 1006 | 4556 | 8106 | None |
| Genewiz | TFORF0917 | NCOA3 | NM_181659, ENST00000371998 | 1007 | 4557 | 8107 | None |
| Genewiz | TFORF0918 | NCOA1 | XM_017005169, XM_017005168, XM_005264628, | 1008 | 4558 | 8108 | None |
| | | | NM_147223, ENST00000405141, ENST00000288599 | | | | |
| Genewiz | TFORF0919 | NCOA1 | XM_005264625, NM_003743, ENST00000406961, | 1009 | 4559 | 8109 | None |
| | | | ENST00000348332 | | | | |
| Genewiz | TFORF0920 | NCOA1 | XM_005264626, NM_147233, ENST00000395856 | 1010 | 4560 | 8110 | None |
| Genewiz | TFORF0921 | BSX | NM_001098169, ENST00000343035 | 1011 | 4561 | 8111 | None |
| Genewiz | TFORF0922 | NR2F1 | NM_005654, ENST00000327111 | 1012 | 4562 | 8112 | None |
| Genewiz | TFORF0923 | NR2F2 | NM_001145155, ENST00000421109 | 1013 | 4563 | 8113 | None |
| Genewiz | TFORF0924 | NR2F2 | NM_021005, ENST00000394166 | 1014 | 4564 | 8114 | None |
| Genewiz | TFORF0925 | NR2F2 | NM_001145156, NM_001145157, ENST00000394171, | 1015 | 4565 | 8115 | None |
| | | | ENST00000453270 | | | | |
| Genewiz | TFORF0926 | ATOH7 | NM_145178, ENST00000373673 | 1016 | 4566 | 8116 | None |
| Genewiz | TFORF0927 | DDB2 | NM_001300734, ENST00000378600 | 1017 | 4567 | 8117 | None |
| Genewiz | TFORF0928 | DDB1 | NM_001923, ENST00000301764 | 1018 | 4568 | 8118 | None |
| Genewiz | TFORF0929 | ATOH8 | NM_032827, ENST00000306279 | 1019 | 4569 | 8119 | None |
| Genewiz | TFORF0930 | ZNF83 | NM_001277951, NM_001105549, NM_001105551, | 1020 | 4570 | 8120 | None |
| | | | NM_001277952, NM_001105550, NM_001105552, | | | | |
| | | | NM_018300, NM_001277947, NM_001277945, | | | | |
| | | | NM_001277949, NM_001277946, NM_001277948, | | | | |
| | | | XM_017026951, ENST00000301096, ENST00000536937, | | | | |
| | | | ENST00000597597, ENST00000541777, ENST00000545872 | | | | |
| Genewiz | TFORF0931 | MECP2 | NM_001110792, ENST00000453960 | 1021 | 4571 | 8121 | None |
| Genewiz | TFORF0932 | ZNF80 | XM_017007133, XM_017007134, XM_017007136, | 1022 | 4572 | 8122 | None |
| | | | XM_017007132, XM_017007135, NM_007136, | | | | |
| | | | ENST00000482457, ENST00000308095, ENST00000619534 | | | | |
| Genewiz | TFORF0933 | ZNF85 | NM_001256172, ENST00000300540 | 1023 | 4573 | 8123 | None |
| Genewiz | TFORF0934 | ZNF85 | NM_003429, ENST00000328178 | 1024 | 4574 | 8124 | None |
| Genewiz | TFORF0935 | ZNF84 | NM_003428, NM_001289971, NM_001127372, | 1025 | 4575 | 8125 | None |
| | | | XM_005266185, XM_005266186, XM_011534832, | | | | |
| | | | NM_001289972, ENST00000392319, ENST00000539354, | | | | |
| | | | ENST00000327668 | | | | |
| Genewiz | TFORF0936 | SS18 | NM_005637, ENST00000269137 | 1026 | 4576 | 8126 | None |
| Genewiz | TFORF0937 | SS18 | NM_001308201, XM_011526145, ENST00000542420 | 1027 | 4577 | 8127 | None |
| Genewiz | TFORF0938 | SS18 | NM_001007559, ENST00000415083 | 1028 | 4578 | 8128 | None |
| Genewiz | TFORF0939 | ZNF473 | NM_001308424, ENST00000445728 | 1029 | 4579 | 8129 | None |
| Genewiz | TFORF0940 | ZNF639 | XM_017006551, NM_001303426, NM_001303425, | 1030 | 4580 | 8130 | None |
| | | | XM_017006552, XM_017006550, XM_017006553, | | | | |
| | | | NM_016331, ENST00000496856, ENST0326361, | | | | |
| | | | ENST00000484866, ENST00000621687 | | | | |
| Genewiz | TFORF0941 | LMO1 | XM_011520099, XM_011520098, ENST00000534484 | 1031 | 4581 | 8131 | None |
| Genewiz | TFORF0942 | LMO1 | NM_001270428, ENST00000428101 | 1032 | 4582 | 8132 | None |
| Genewiz | TFORF0943 | LMO2 | NM_005574, ENST00000257818 | 1033 | 4583 | 8133 | None |
| Genewiz | TFORF0944 | LMO3 | NM_001243612, ENST00000541295 | 1034 | 4584 | 8134 | None |
| Genewiz | TFORF0945 | LMO3 | NM_001243613, ENST00000540445 | 1035 | 4585 | 8135 | None |
| Genewiz | TFORF0946 | LMO3 | NM_001243611, ENST00000261169 | 1036 | 4586 | 8136 | None |
| Genewiz | TFORF0947 | EMX2 | NM_001165924, ENST00000442245 | 1037 | 4587 | 8137 | None |
| Genewiz | TFORF0948 | EMX2 | NM_004098, ENST00000553456 | 1038 | 4588 | 8138 | None |
| Genewiz | TFORF0949 | EMX1 | NM_004097, ENST00000258106 | 1039 | 4589 | 8139 | None |
| Genewiz | TFORF0950 | PBRM1 | XM_017006765, ENST00000337303 | 1040 | 4590 | 8140 | None |
| Genewiz | TFORF0951 | PBRM1 | XM_017006748, XM_017006749, XM_017006750, | 1041 | 4591 | 8141 | None |
| | | | ENST00000296302 | | | | |
| Genewiz | TFORF0952 | PBRM1 | NM_018313, ENST00000394830 | 1042 | 4592 | 8142 | None |
| Genewiz | TFORF0953 | PBRM1 | XM_017006758, XM_017006757, ENST00000409057 | 1043 | 4593 | 8143 | None |
| Genewiz | TFORF0954 | CDIP1 | NM_001199055, ENST00000563507 | 1044 | 4594 | 8144 | None |
| Genewiz | TFORF0955 | CDIP1 | NM_001199056, ENST00000562334 | 1045 | 4595 | 8145 | None |
| Genewiz | TFORF0956 | MYF5 | NM_005593, ENST00000228644 | 1046 | 4596 | 8146 | None |
| Genewiz | TFORF0957 | ZNF497 | NM_198458, NM_001207009, ENST00000311044, | 1047 | 4597 | 8147 | None |
| | | | ENST00000425453 | | | | |
| Genewiz | TFORF0958 | ZNF496 | XM_005273330, NM_032752, ENST00000294753 | 1048 | 4598 | 8148 | None |
| Genewiz | TFORF0959 | ERF | NM_001308402, NM_001301035, NM_001312656, | 1049 | 4599 | 8149 | None |
| | | | XM_017026468, XM_017026469, ENST00000440177 | | | | |
| Genewiz | TFORF0960 | ERG | NM_001243428, NM_001136154, ENST00000417133, | 1050 | 4600 | 8150 | None |
| | | | ENST00000398919 | | | | |
| Genewiz | TFORF0961 | ERG | NM_001243429, ENST00000398897 | 1051 | 4601 | 8151 | None |
| Genewiz | TFORF0962 | ERG | NM_004449, ENST00000398911, ENST00000442448 | 1052 | 4602 | 8152 | None |
| Genewiz | TFORF0963 | ERG | NM_001136155, ENST00000453032 | 1053 | 4603 | 8153 | None |
| Genewiz | TFORF0964 | ERG | XM_017028288, ENST00000398905 | 1054 | 4604 | 8154 | None |
| Genewiz | TFORF0965 | ZNF493 | NM_175910, ENST00000355504 | 1055 | 4605 | 8155 | None |
| Genewiz | TFORF0966 | ZNF493 | NM_145326, ENST00000339914 | 1056 | 4606 | 8156 | None |
| Genewiz | TFORF0967 | ZNF493 | NM_001076678, ENST00000392288 | 1057 | 4607 | 8157 | None |
| Genewiz | TFORF0968 | HAND1 | NM_004821, ENST00000231121 | 1058 | 4608 | 8158 | None |
| Genewiz | TFORF0969 | HAND2 | NM_021973, ENST00000359562 | 1059 | 4609 | 8159 | None |
| Genewiz | TFORF0970 | ZNF124 | NM_001243740, ENST00000472531 | 1060 | 4610 | 8160 | None |
| Genewiz | TFORF0971 | ZNF124 | NM_003431, ENST00000340684 | 1061 | 4611 | 8161 | None |
| Genewiz | TFORF0972 | ZNF124 | NM_001297567, ENST00000491356 | 1062 | 4612 | 8162 | None |
| Genewiz | TFORF0973 | ZNF124 | NM_001297568, ENST00000543802 | 1063 | 4613 | 8163 | None |
| Genewiz | TFORF0974 | GFI1B | XM_011519068, XM_017015175, XM_011519069, | 1064 | 4614 | 8164 | None |
| | | | XM_006717297, ENST00000636137 | | | | |
| Genewiz | TFORF0975 | GFI1B | NM_004188, XM_011519070, ENST00000339463, | 1065 | 4615 | 8165 | None |
| | | | ENST00000372122 | | | | |
| Genewiz | TFORF0976 | GFI1B | XM_017015176, NM_001135031, ENST00000372123 | 1066 | 4616 | 8166 | None |
| Genewiz | TFORF0977 | ZNF121 | XM_017027239, NM_001308269, NM_001008727, | 1067 | 4617 | 8167 | None |
| | | | ENST00000586602, ENST00000320451 | | | | |
| Genewiz | TFORF0978 | KLF7 | NM_001270944, ENST00000412414 | 1068 | 4618 | 8168 | None |
| Genewiz | TFORF0979 | KLF7 | NM_003709, XM_017005161, ENST00000309446 | 1069 | 4619 | 8169 | None |
| Genewiz | TFORF0980 | KLF7 | NM_001270943, ENST00000421199 | 1070 | 4620 | 8170 | None |
| Genewiz | TFORF0981 | KLF6 | NM_001300, ENST00000497571 | 1071 | 4621 | 8171 | None |
| Genewiz | TFORF0982 | KLF6 | NM_001160125, ENST00000542957 | 1072 | 4622 | 8172 | None |
| Genewiz | TFORF0983 | KLF5 | NM_001730, ENST00000377687 | 1073 | 4623 | 8173 | None |
| Genewiz | TFORF0984 | KLF5 | NM_001286818, ENST00000539231 | 1074 | 4624 | 8174 | None |
| Genewiz | TFORF0985 | KLF4 | NM_004235, ENST00000374672 | 1075 | 4625 | 8175 | None |
| Genewiz | TFORF0986 | GZF1 | XM_011529321, XM_011529322, NM_022482, | 1076 | 4626 | 8176 | None |
| | | | NM_001317012, ENST00000338121, ENST00000377051 | | | | |
| Genewiz | TFORF0987 | KLF2 | NM_016270, ENST00000248071 | 1077 | 4627 | 8177 | None |
| Genewiz | TFORF0988 | KLF1 | NM_006563, ENST00000264834 | 1078 | 4628 | 8178 | None |
| Genewiz | TFORF0989 | KLF9 | NM_001206, ENST00000377126 | 1079 | 4629 | 8179 | None |
| Genewiz | TFORF0990 | KLF8 | XM_006724576, ENST00000358094 | 1080 | 4630 | 8180 | None |
| Genewiz | TFORF0991 | KLF8 | XM_005261979, XM_005261977, NM_001324102, | 1081 | 4631 | 8181 | None |
| | | | NM_007250, ENST00000468660 | | | | |
| Genewiz | TFORF0992 | KLF8 | NM_001324099, ENST00000640927 | 1082 | 4632 | 8182 | None |
| Genewiz | TFORF0993 | KLF8 | NM_001159296, XM_006724575, ENST00000374928 | 1083 | 4633 | 8183 | None |
| Genewiz | TFORF0994 | ZNF304 | NM_020657, ENST00000391705, ENST00000282286 | 1084 | 4634 | 8184 | None |
| Genewiz | TFORF0995 | ZNF304 | NM_001290318, ENST00000443917 | 1085 | 4635 | 8185 | None |
| Genewiz | TFORF0996 | ZNF304 | NM_001290319, ENST00000598744 | 1086 | 4636 | 8186 | None |
| Genewiz | TFORF0997 | ZNF302 | XM_011527111, NM_001289189, NM_001289188, | 1087 | 4637 | 8187 | None |
| | | | XM_017026986, ENST00000507959 | | | | |
| Genewiz | TFORF0998 | ZNF302 | NM_001289190, NM_001289191, NM_001289192, | 1088 | 4638 | 8188 | None |
| | | | NM_018675, ENST00000505365 | | | | |
| Genewiz | TFORF0999 | ZNF302 | NM_001289185, NM_001289184, NM_001289182, | 1089 | 4639 | 8189 | None |
| | | | XM_017026982, ENST00000613363 | | | | |
| Genewiz | TFORF1000 | ZNF302 | NM_001289181, XM_017026979, XM_017026981, | 1090 | 4640 | 8190 | None |
| | | | XM_017026980, ENST00000446502 | | | | |
| Genewiz | TFORF1001 | REST | NM_005612, NM_001193508, XM_011534401, | 1091 | 4641 | 8191 | None |
| | | | ENST00000309042, ENST00000619101 | | | | |
| Genewiz | TFORF1002 | ZNF300 | NM_001172832, ENST00000418587 | 1092 | 4642 | 8192 | None |
| Genewiz | TFORF1003 | ZNF300 | NM_001172831, ENST00000446148 | 1093 | 4643 | 8193 | None |
| Genewiz | TFORF1004 | POU1F1 | NM_001122757, ENST00000344265 | 1094 | 4644 | 8194 | None |
| Genewiz | TFORF1005 | POU1F1 | NM_000306, ENST00000350375 | 1095 | 4645 | 8195 | None |
| Genewiz | TFORF1006 | ZNF564 | NM_144976, ENST00000339282 | 1096 | 4646 | 8196 | None |
| Genewiz | TFORF1007 | ZNF544 | NM_001320771, NM_001320770, NM_001320773, | 1097 | 4647 | 8197 | None |
| | | | ENST00000600044, ENST00000600220 | | | | |
| Genewiz | TFORF1008 | ZNF544 | NM_001320788, NM_001320792, NM_001320791, | 1098 | 4648 | 8198 | None |
| | | | NM_001320789, ENST00000599227, ENST00000594384, | | | | |
| | | | ENST00000596825 | | | | |
| Genewiz | TFORF1009 | ZNF544 | NM_001320782, ENST00000596929 | 1099 | 4649 | 8199 | None |
| Genewiz | TFORF1010 | ZNF547 | NM_173631, ENST00000282282 | 1100 | 4650 | 8200 | None |
| Genewiz | TFORF1011 | ZNF546 | NM_178544, XM_011526899, ENST00000347077 | 1101 | 4651 | 8201 | None |
| Genewiz | TFORF1012 | ZNF546 | NM_001297763, ENST00000600094 | 1102 | 4652 | 8202 | None |
| Genewiz | TFORF1013 | NCL | NM_005381, ENST00000322723 | 1103 | 4653 | 8203 | None |
| Genewiz | TFORF1014 | ZNF540 | NM_001172226, ENST00000589117 | 1104 | 4654 | 8204 | None |
| Genewiz | TFORF1015 | ZNF540 | NM_152606, NM_001172225, ENST00000592533, | 1105 | 4655 | 8205 | None |
| | | | ENST00000316433, ENST00000343599 | | | | |
| Genewiz | TFORF1016 | ZNF549 | NM_153263, ENST00000240719 | 1106 | 4656 | 8206 | None |
| Genewiz | TFORF1017 | ZNF548 | NM_001172773, ENST00000336128 | 1107 | 4657 | 8207 | None |
| Genewiz | TFORF1018 | ZNF548 | NM_152909, ENST00000366197 | 1108 | 4658 | 8208 | None |
| Genewiz | TFORF1019 | SCRT1 | NM_031309, ENST00000569446 | 1109 | 4659 | 8209 | None |
| Genewiz | TFORF1020 | ZXDC | NM_025112, ENST00000389709 | 1110 | 4660 | 8210 | None |
| Genewiz | TFORF1021 | ZXDC | NM_001040653, ENST00000336332 | 1111 | 4661 | 8211 | None |
| Genewiz | TFORF1022 | SCRT2 | NM_033129, ENST00000246104 | 1112 | 4662 | 8212 | None |
| Genewiz | TFORF1023 | FOXI1 | NM_012188, ENST00000306268 | 1113 | 4663 | 8213 | None |
| Genewiz | TFORF1024 | FOXI3 | NM_001135649, ENST00000428390 | 1114 | 4664 | 8214 | None |
| Genewiz | TFORF1025 | FOXI2 | NM_207426, ENST00000388920 | 1115 | 4665 | 8215 | None |
| Genewiz | TFORF1026 | RARA | NM_001024809, ENST00000394081 | 1116 | 4666 | 8216 | None |
| Genewiz | TFORF1027 | RARA | XM_005257552, ENST00000394086 | 1117 | 4667 | 8217 | None |
| Genewiz | TFORF1028 | RARA | NM_001145302, ENST00000425707 | 1118 | 4668 | 8218 | None |
| Genewiz | TFORF1029 | RARB | NM_016152, NM_001290276, NM_001290217, | 1119 | 4669 | 8219 | None |
| | | | ENST00000437042, ENST00000458646 | | | | |
| Genewiz | TFORF1030 | RARG | NM_001243732, ENST00000543726 | 1120 | 4670 | 8220 | None |
| Genewiz | TFORF1031 | RARG | NM_001042728, ENST00000338561 | 1121 | 4671 | 8221 | None |
| Genewiz | TFORF1032 | RARG | NM_001243730, ENST00000394426 | 1122 | 4672 | 8222 | None |
| Genewiz | TFORF1033 | MYT1 | NM_004535, ENST00000328439 | 1123 | 4673 | 8223 | None |
| Genewiz | TFORF1034 | WT1 | NM_000378, ENST00000452863, ENST00000639563 | 1124 | 4674 | 8224 | None |
| Genewiz | TFORF1035 | WT1 | NM_001198552, ENST00000530998 | 1125 | 4675 | 8225 | None |
| Genewiz | TFORF1036 | WT1 | NM_024426, ENST00000332351, ENST00000640146 | 1126 | 4676 | 8226 | None |
| Genewiz | TFORF1037 | WT1 | NM_024424, ENST00000448076, ENST00000639907 | 1127 | 4677 | 8227 | None |
| Genewiz | TFORF1038 | WT1 | NM_001198551, ENST00000379079 | 1128 | 4678 | 8228 | None |
| Genewiz | TFORF1039 | TSC22D1 | NM_001243799, ENST00000501704 | 1129 | 4679 | 8229 | None |
| Genewiz | TFORF1040 | TSC22D1 | NM_006022, ENST00000261489 | 1130 | 4680 | 8230 | None |
| Genewiz | TFORF1041 | TSC22D1 | NM_183422, ENST00000458659 | 1131 | 4681 | 8231 | None |
| Genewiz | TFORF1042 | TSC22D1 | NM_001243797, NM_001243798, ENST00000622051, | 1132 | 4682 | 8232 | None |
| | | | ENST00000611198 | | | | |
| Genewiz | TFORF1043 | TSC22D3 | NM_004089, ENST00000372397 | 1133 | 4683 | 8233 | None |
| Genewiz | TFORF1044 | TSC22D3 | NM_198057, NM_001318468, NM_001318470, | 1134 | 4684 | 8234 | None |
| | | | XM_017029335, XM_005262100, XM_005262102, | | | | |
| | | | XM_005262103, XM_011530884, XM_005262099, | | | | |
| | | | ENST00000315660, ENST00000372383, ENST00000372384, | | | | |
| | | | ENST00000506081 | | | | |
| Genewiz | TFORF1045 | TSC22D2 | NM_014779, ENST00000361875 | 1135 | 4685 | 8235 | None |
| Genewiz | TFORF1046 | BAZ1B | NM_032408, XM_017012773, ENST00000339594, | 1136 | 4686 | 8236 | None |
| | | | ENST00000404251 | | | | |
| Genewiz | TFORF1047 | SNAI2 | NM_003068, ENST00000020945 | 1137 | 4687 | 8237 | None |
| Genewiz | TFORF1048 | PLAGL2 | NM_002657, XM_005260436, XM_011528864, | 1138 | 4688 | 8238 | None |
| | | | XM_011528863, ENST00000246229 | | | | |
| Genewiz | TFORF1049 | HKR1 | XM_017026677, XM_017026676, ENST00000589392 | 1139 | 4689 | 8239 | None |
| Genewiz | TFORF1050 | HKR1 | XM_017026686, XM_017026689, XM_017026690, | 1140 | 4690 | 8240 | None |
| | | | XM_017026687, XM_017026688, ENST00000541583 | | | | |
| Genewiz | TFORF1051 | HKR1 | NM_181786, ENST00000324411 | 1141 | 4691 | 8241 | None |
| Genewiz | TFORF1052 | HKR1 | XM_017026679, XM_017026678, ENST00000392153 | 1142 | 4692 | 8242 | None |
| Genewiz | TFORF1053 | HKR1 | XM_017026698, XM_017026702, XM_017026701, | 1143 | 4693 | 8243 | None |
| | | | XM_017026695, XM_017026692, XM_017026693, | | | | |
| | | | XM_017026700, XM_017026694, XM_017026704, | | | | |
| | | | XM_017026703, XM_017026697, XM_017026696, | | | | |
| | | | XM_017026691, XM_017026699, XM_017026705, | | | | |
| | | | ENST00000591471, ENST00000544914 | | | | |
| Genewiz | TFORF1054 | PLAGL1 | NM_001317159, NM_001080951, NM_001289043, | 1144 | 4694 | 8244 | None |
| | | | NM_001289044, NM_001289042, NM_001317157, | | | | |
| | | | NM_001317156, NM_001289046, NM_001317162, | | | | |
| | | | NM_001080954, NM_001080952, NM_006718, | | | | |
| | | | NM_001289048, NM_001289045, NM_001317161, | | | | |
| | | | NM_001080953, NM_001289049, NM_001289047, | | | | |
| | | | ENST00000360537, ENST00000354765, ENST0416623, | | | | |
| | | | ENST00000444202, ENST00000625622, ENST00000367571 | | | | |
| Genewiz | TFORF1055 | PLAGL1 | NM_001289039, NM_001289040, NM_001080955, | 1145 | 4695 | 8245 | None |
| | | | NM_001289037, NM_001289038, NM_001317158, | | | | |
| | | | NM_001080956, NM_001289041, NM_001317160, | | | | |
| | | | ENST00000437412, ENST00000367572, ENST00000417959 | | | | |
| Genewiz | TFORF1056 | NFATC1 | NM_001278673, ENST00000545796 | 1146 | 4696 | 8246 | None |
| Genewiz | TFORF1057 | NFATC1 | NM_001278675, ENST00000592223 | 1147 | 4697 | 8247 | None |
| Genewiz | TFORF1058 | NFATC1 | NM_172389, ENST00000318065 | 1148 | 4698 | 8248 | None |
| Genewiz | TFORF1059 | NFATC1 | NM_006162, ENST00000253506 | 1149 | 4699 | 8249 | None |
| Genewiz | TFORF1060 | NFATC1 | NM_001278672, ENST00000586434 | 1150 | 4700 | 8250 | None |
| Genewiz | TFORF1061 | NFATC1 | NM_001278670, ENST00000542384 | 1151 | 4701 | 8251 | None |
| Genewiz | TFORF1062 | NFATC1 | NM_001278669, ENST00000427363 | 1152 | 4702 | 8252 | None |
| Genewiz | TFORF1063 | NFATC1 | NM_172388, ENST00000397790 | 1153 | 4703 | 8253 | None |
| Genewiz | TFORF1064 | NFATC1 | NM_172387, ENST00000329101 | 1154 | 4704 | 8254 | None |
| Genewiz | TFORF1065 | NFATC2 | NM_001258296, NM_001258294, ENST00000610033, | 1155 | 4705 | 8255 | None |
| | | | ENST00000609507 | | | | |
| Genewiz | TFORF1066 | NFATC2 | NM_173091, ENST00000396009 | 1156 | 4706 | 8256 | None |
| Genewiz | TFORF1067 | NFATC2 | NM_001258292, ENST00000609943 | 1157 | 4707 | 8257 | None |
| Genewiz | TFORF1068 | NFATC2 | NM_001136021, ENST00000414705 | 1158 | 4708 | 8258 | None |
| Genewiz | TFORF1069 | NFATC2 | NM_012340, ENST00000371564 | 1159 | 4709 | 8259 | None |
| Genewiz | TFORF1070 | NFATC3 | NM_004555, ENST00000329524 | 1160 | 4710 | 8260 | None |
| Genewiz | TFORF1071 | NFATC3 | NM_173163, ENST00000349223 | 1161 | 4711 | 8261 | None |
| Genewiz | TFORF1072 | NFATC4 | NM_001136022, ENST00000413692 | 1162 | 4712 | 8262 | None |
| Genewiz | TFORF1073 | NFATC4 | XM_011536797, ENST00000555453 | 1163 | 4713 | 8263 | None |
| Genewiz | TFORF1074 | NFATC4 | NM_001198966, ENST00000553879, ENST00000554344 | 1164 | 4714 | 8264 | None |
| Genewiz | TFORF1075 | NFATC4 | NM_001198965, ENST00000554050 | 1165 | 4715 | 8265 | None |
| Genewiz | TFORF1076 | NFATC4 | NM_001288802, ENST00000422617 | 1166 | 4716 | 8266 | None |
| Genewiz | TFORF1077 | NFATC4 | XM_011536799, ENST00000556169 | 1167 | 4717 | 8267 | None |
| Genewiz | TFORF1078 | NFATC4 | NM_001198967, ENST00000554591 | 1168 | 4718 | 8268 | None |
| Genewiz | TFORF1079 | NFATC4 | NM_004554, ENST00000250373 | 1169 | 4719 | 8269 | None |
| Genewiz | TFORF1080 | NOBOX | NM_001080413, ENST00000467773 | 1170 | 4720 | 8270 | None |
| Genewiz | TFORF1081 | NOBOX | XM_017011742, ENST00000483238 | 1171 | 4721 | 8271 | None |
| Genewiz | TFORF1082 | FUBP1 | NM_003902, ENST00000370768 | 1172 | 4722 | 8272 | None |
| Genewiz | TFORF1083 | FUBP1 | XM_017002743, ENST00000294623 | 1173 | 4723 | 8273 | None |
| Genewiz | TFORF1084 | SPZ1 | NM_032567, ENST00000296739 | 1174 | 4724 | 8274 | None |
| Genewiz | TFORF1085 | AP2B1 | NM_001282, XM_017024284, ENST00000621914 | 1175 | 4725 | 8275 | None |
| Genewiz | TFORF1086 | GTF2E1 | NM_005513, XM_011512745, XM_011512744, | 1176 | 4726 | 8276 | None |
| | | | ENST00000283875 | | | | |
| Genewiz | TFORF1087 | TULP3 | NM_001160408, ENST00000397132 | 1177 | 4727 | 8277 | None |
| Genewiz | TFORF1088 | TULP3 | NM_003324, ENST00000448120 | 1178 | 4728 | 8278 | None |
| Genewiz | TFORF1089 | TULP1 | NM_003322, ENST00000229771 | 1179 | 4729 | 8279 | None |
| Genewiz | TFORF1090 | TULP1 | NM_001289395, ENST00000322263 | 1180 | 4730 | 8280 | None |
| Genewiz | TFORF1091 | ZBTB8A | NM_001291496, ENST00000316459 | 1181 | 4731 | 8281 | None |
| Genewiz | TFORF1092 | ZBTB8A | NM_001040441, ENST00000373510 | 1182 | 4732 | 8282 | None |
| Genewiz | TFORF1093 | ZBTB8B | NM_001145720, ENST00000609129 | 1183 | 4733 | 8283 | None |
| Genewiz | TFORF1094 | HSFX2 | NM_001164415, ENST00000598963 | 1184 | 4734 | 8284 | None |
| Genewiz | TFORF1095 | EN1 | NM_001426, ENST00000295206 | 1185 | 4735 | 8285 | None |
| Genewiz | TFORF1096 | EN2 | NM_001427, ENST00000297375 | 1186 | 4736 | 8286 | None |
| Genewiz | TFORF1097 | HSFX1 | NM_016153, ENST00000370416 | 1187 | 4737 | 8287 | None |
| Genewiz | TFORF1098 | UBP1 | NM_001128160, ENST00000447368 | 1188 | 4738 | 8288 | None |
| Genewiz | TFORF1099 | UBP1 | NM_014517, NM_001128161, ENST00000283629, | 1189 | 4739 | 8289 | None |
| | | | ENST00000283628 | | | | |
| Genewiz | TFORF1100 | GTF2IRD1 | XM_006716182, XM_006716183, ENST00000476977 | 1190 | 4740 | 8290 | None |
| Genewiz | TFORF1101 | GTF2IRD1 | NM_005685, XM_017012804, ENST00000424337 | 1191 | 4741 | 8291 | None |
| Genewiz | TFORF1102 | GTF2IRD1 | NM_016328, ENST00000265755 | 1192 | 4742 | 8292 | None |
| Genewiz | TFORF1103 | GTF2IRD1 | NM_001199207, ENST00000455841 | 1193 | 4743 | 8293 | None |
| Genewiz | TFORF1104 | GTF2IRD2 | NM_173537, ENST00000451013 | 1194 | 4744 | 8294 | None |
| Genewiz | TFORF1105 | GTF2IRD2 | NM_001281447, ENST00000614386 | 1195 | 4745 | 8295 | None |
| Genewiz | TFORF1106 | EPAS1 | NM_001430, ENST00000263734 | 1196 | 4746 | 8296 | None |
| Genewiz | TFORF1107 | ZNF37A | NM_001324258, NM_001324256, NM_001324257, | 1197 | 4747 | 8297 | None |
| | | | XM_017016621, ENST00000638053 | | | | |
| Genewiz | TFORF1108 | CREBBP | NM_004380, ENST00000262367 | 1198 | 4748 | 8298 | None |
| Genewiz | TFORF1109 | CREBBP | NM_001079846, ENST00000382070 | 1199 | 4749 | 8299 | None |
| Genewiz | TFORF1110 | ISX | NM_001303508, ENST00000404699, ENST00000308700 | 1200 | 4750 | 8300 | None |
| Genewiz | TFORF1111 | RCOR3 | NM_001136225, ENST00000452621 | 1201 | 4751 | 8301 | None |
| Genewiz | TFORF1112 | RCOR3 | NM_018254, ENST00000367005 | 1202 | 4752 | 8302 | None |
| Genewiz | TFORF1113 | RCOR3 | NM_001136223, ENST00000419091 | 1203 | 4753 | 8303 | None |
| Genewiz | TFORF1114 | RCOR3 | NM_001136224, ENST00000367006 | 1204 | 4754 | 8304 | None |
| Genewiz | TFORF1115 | RCOR2 | NM_173587, ENST00000301459 | 1205 | 4755 | 8305 | None |
| Genewiz | TFORF1116 | RCOR1 | NM_015156, ENST00000262241 | 1206 | 4756 | 8306 | None |
| Genewiz | TFORF1117 | BRIP1 | NM_032043, ENST00000259008 | 1207 | 4757 | 8307 | None |
| Genewiz | TFORF1118 | SKP2 | NM_001243120, ENST00000620197 | 1208 | 4758 | 8308 | None |
| Genewiz | TFORF1119 | ZNF714 | NM_182515, ENST00000456283, ENST00000610902 | 1209 | 4759 | 8309 | None |
| Genewiz | TFORF1120 | IFI16 | NM_001206567, ENST00000359709 | 1210 | 4760 | 8310 | None |
| Genewiz | TFORF1121 | IFI16 | NM_005531, ENST00000368131, ENST00000368132 | 1211 | 4761 | 8311 | None |
| Genewiz | TFORF1122 | HNRNPAB | NM_004499, ENST00000355836, ENST00000506259 | 1212 | 4762 | 8312 | None |
| Genewiz | TFORF1123 | HNRNPAB | NM_031266, ENST00000358344, ENST00000504898 | 1213 | 4763 | 8313 | None |
| Genewiz | TFORF1124 | E2F7 | NM_203394, ENST00000322886 | 1214 | 4764 | 8314 | None |
| Genewiz | TFORF1125 | ENO1 | NM_001428, ENST00000234590 | 1215 | 4765 | 8315 | None |
| Genewiz | TFORF1126 | THAP3 | XM_005263532, NM_001195752, ENST00000307896 | 1216 | 4766 | 8316 | None |
| Genewiz | TFORF1127 | THAP3 | NM_138350, ENST00000377627 | 1217 | 4767 | 8317 | None |
| Genewiz | TFORF1128 | THAP3 | NM_001195753, ENST00000054650 | 1218 | 4768 | 8318 | None |
| Genewiz | TFORF1129 | ZNF808 | XM_005258909, ENST00000487863 | 1219 | 4769 | 8319 | None |
| Genewiz | TFORF1130 | ZNF808 | NM_001321425, NM_001039886, NM_001321424, | 1220 | 4770 | 8320 | None |
| | | | ENST00000359798 | | | | |
| Genewiz | TFORF1131 | BNC1 | NM_001717, ENST00000345382 | 1221 | 4771 | 8321 | None |
| Genewiz | TFORF1132 | BNC1 | NM_001301206, ENST00000569704 | 1222 | 4772 | 8322 | None |
| Genewiz | TFORF1133 | CARF | NM_001104586, NM_024744, NM_001322427, | 1223 | 4773 | 8323 | None |
| | | | XM_011511867, XM_005246859, XM_005246858, | | | | |
| | | | XM_017004961, NM_001322428, ENST0402905, | | | | |
| | | | ENST00000438828 | | | | |
| Genewiz | TFORF1134 | CARF | XM_017004964, XM_017004965, ENST00000414439 | 1224 | 4774 | 8324 | None |
| Genewiz | TFORF1135 | CARF | NM_001282910, NM_001282911, ENST00000320443, | 1225 | 4775 | 8325 | None |
| | | | ENST00000428585 | | | | |
| Genewiz | TFORF1136 | LRRFIP1 | NM_001137552, ENST00000392000 | 1226 | 4776 | 8326 | None |
| Genewiz | TFORF1137 | LRRFIP1 | NM_001137550, ENST00000308482 | 1227 | 4777 | 8327 | None |
| Genewiz | TFORF1138 | LRRFIP1 | NM_001137553, ENST00000289175 | 1228 | 4778 | 8328 | None |
| Genewiz | TFORF1139 | LRRFIP1 | NM_004735, ENST00000244815 | 1229 | 4779 | 8329 | None |
| Genewiz | TFORF1140 | HMBOX1 | NM_001324391, NM_001324392, ENST00000558662 | 1230 | 4780 | 8330 | None |
| Genewiz | TFORF1141 | HMBOX1 | NM_001135726, NM_024567, NM_001324382, | 1231 | 4781 | 8331 | None |
| | | | ENST00000287701, ENST00000397358 | | | | |
| Genewiz | TFORF1142 | HMBOX1 | XM_005273635, ENST00000524238 | 1232 | 4782 | 8332 | None |
| Genewiz | TFORF1143 | HMBOX1 | XM_017013825, ENST00000521516 | 1233 | 4783 | 8333 | None |
| Genewiz | TFORF1144 | HOXA7 | NM_006896, ENST00000242159 | 1234 | 4784 | 8334 | None |
| Genewiz | TFORF1145 | CDK1 | NM_033379, ENST00000316629, ENST00000373809 | 1235 | 4785 | 8335 | None |
| Genewiz | TFORF1146 | HOXA4 | NM_002141, ENST00000610970, ENST00000360046, | 1236 | 4786 | 8336 | None |
| | | | ENST00000428284 | | | | |
| Genewiz | TFORF1147 | HOXA3 | XM_011515343, XM_006715715, XM_005249730, | 1237 | 4787 | 8337 | None |
| | | | NM_030661, NM_153631, XM_005249731, | | | | |
| | | | XM_005249732, ENST00000396352, ENST012286, | | | | |
| | | | ENST00000317201 | | | | |
| Genewiz | TFORF1148 | HOXA2 | NM_006735, ENST00000222718 | 1238 | 4788 | 8338 | None |
| Genewiz | TFORF1149 | HOXA1 | NM_153620, ENST00000355633 | 1239 | 4789 | 8339 | None |
| Genewiz | TFORF1150 | CDK7 | NM_001324072, NM_001324077, NM_001324078, | 1240 | 4790 | 8340 | None |
| | | | NM_001324074, NM_001324075, ENST00000502604 | | | | |
| Genewiz | TFORF1151 | MYOCD | NM_153604, ENST00000343344 | 1241 | 4791 | 8341 | None |
| Genewiz | TFORF1152 | MYOCD | NM_001146312, ENST00000425538 | 1242 | 4792 | 8342 | None |
| Genewiz | TFORF1153 | RERE | NM_012102, NM_001042681, XM_005263464, | 1243 | 4793 | 8343 | None |
| | | | XM_017001359, XM_017001358, ENST00000337907, | | | | |
| | | | ENST00000400908 | | | | |
| Genewiz | TFORF1154 | RERE | NM_001042682, ENST00000476556 | 1244 | 4794 | 8344 | None |
| Genewiz | TFORF1155 | RERE | XM_005263466, ENST00000377464 | 1245 | 4795 | 8345 | None |
| Genewiz | TFORF1156 | DMBX1 | NM_147192, ENST00000371956 | 1246 | 4796 | 8346 | None |
| Genewiz | TFORF1157 | DMBX1 | XM_017000289, XM_011540668, NM_172225, | 1247 | 4797 | 8347 | None |
| | | | ENST00000360032 | | | | |
| Genewiz | TFORF1158 | EOMES | NM_001278182, ENST00000449599 | 1248 | 4798 | 8348 | None |
| Genewiz | TFORF1159 | EOMES | NM_005442, ENST00000295743 | 1249 | 4799 | 8349 | None |
| Genewiz | TFORF1160 | EOMES | NM_001278183, ENST00000461503 | 1250 | 4800 | 8350 | None |
| Genewiz | TFORF1161 | NFKBIL1 | NM_001144962, ENST00000376146 | 1251 | 4801 | 8351 | None |
| Genewiz | TFORF1162 | NFKBIL1 | NM_005007, ENST00000376148 | 1252 | 4802 | 8352 | None |
| Genewiz | TFORF1163 | NFKBIL1 | NM_001144961, ENST00000376145 | 1253 | 4803 | 8353 | None |
| Genewiz | TFORF1164 | NFXL1 | NM_001278624, NM_152995, NM_001278623, | 1254 | 4804 | 8354 | None |
| | | | ENST00000329043, ENST00000381538, ENST00000507489 | | | | |
| Genewiz | TFORF1165 | PRKCD | NM_212539, NM_006254, XM_006713259, | 1255 | 4805 | 8355 | None |
| | | | NM_001316327, XM_017006856, XM_017006855, | | | | |
| | | | ENST00000394729, ENST00000330452 | | | | |
| Genewiz | TFORF1166 | EBF4 | NM_001110514, ENST00000380648 | 1256 | 4806 | 8356 | None |
| Genewiz | TFORF1167 | EBF3 | XM_005252668, ENST00000355311 | 1257 | 4807 | 8357 | None |
| Genewiz | TFORF1168 | EBF2 | NM_022659, ENST00000520164 | 1258 | 4808 | 8358 | None |
| Genewiz | TFORF1169 | EBF1 | NM_182708, ENST00000380654 | 1259 | 4809 | 8359 | None |
| Genewiz | TFORF1170 | MLX | NM_198205, ENST00000346833 | 1260 | 4810 | 8360 | None |
| Genewiz | TFORF1171 | MLX | NM_170607, ENST00000246912 | 1261 | 4811 | 8361 | None |
| Genewiz | TFORF1172 | ZNF157 | NM_003446, ENST00000377073 | 1262 | 4812 | 8362 | None |
| Genewiz | TFORF1173 | ZNF154 | NM_001085384, ENST00000512439, ENST00000451275 | 1263 | 4813 | 8363 | None |
| Genewiz | TFORF1174 | NRL | XM_011536804, XM_011536805, XM_005267709, | 1264 | 4814 | 8364 | None |
| | | | XM_011536802, XM_005267710, XM_005267708, | | | | |
| | | | NM_006177, ENST00000397002, ENST0561028, | | | | |
| | | | ENST00000396997 | | | | |
| Genewiz | TFORF1175 | ZNF790 | XM_011526950, XM_005258903, NM_001242802, | 1265 | 4815 | 8365 | None |
| | | | NM_206894, NM_001242801, NM_001242800, | | | | |
| | | | ENST00000356725, ENST00000615484, ENST00000614179, | | | | |
| | | | ENST00000613249 | | | | |
| Genewiz | TFORF1176 | ZNF792 | NM_175872, ENST00000404801 | 1266 | 4816 | 8366 | None |
| Genewiz | TFORF1177 | ZNF793 | NM_001013659, XM_006723213, XM_005258927, | 1267 | 4817 | 8367 | None |
| | | | ENST00000587143, ENST00000445217 | | | | |
| Genewiz | TFORF1178 | ZNF98 | NM_001098626, ENST00000357774 | 1268 | 4818 | 8368 | None |
| Genewiz | TFORF1179 | ZNF799 | NM_001080821, ENST00000430385 | 1269 | 4819 | 8369 | None |
| Genewiz | TFORF1180 | ZNF799 | NM_001322497, NM_001322498, ENST00000419318 | 1270 | 4820 | 8370 | None |
| Genewiz | TFORF1181 | TBX21 | NM_013351, ENST00000177694 | 1271 | 4821 | 8371 | None |
| Genewiz | TFORF1182 | TBX20 | NM_001077653, ENST00000408931 | 1272 | 4822 | 8372 | None |
| Genewiz | TFORF1183 | DEAF1 | NM_021008, ENST00000382409 | 1273 | 4823 | 8373 | None |
| Genewiz | TFORF1184 | MAP3K7 | NM_145331, ENST00000369329 | 1274 | 4824 | 8374 | None |
| Genewiz | TFORF1185 | MAP3K7 | NM_145332, ENST00000369325 | 1275 | 4825 | 8375 | None |
| Genewiz | TFORF1186 | MAP3K7 | NM_003188, ENST00000369332 | 1276 | 4826 | 8376 | None |
| Genewiz | TFORF1187 | ZNF570 | NM_001300993, ENST00000586475 | 1277 | 4827 | 8377 | None |
| Genewiz | TFORF1188 | ZNF570 | NM_144694, NM_001321991, XM_011526544, | 1278 | 4828 | 8378 | None |
| | | | ENST00000330173 | | | | |
| Genewiz | TFORF1189 | ZNF571 | NM_001321272, NM_001290314, NM_016536, | 1279 | 4829 | 8379 | None |
| | | | ENST00000328550, ENST00000593133, ENST00000451802, | | | | |
| | | | ENST00000358744 | | | | |
| Genewiz | TFORF1190 | ZNF573 | NM_001172690, ENST00000590414, ENST00000536220 | 1280 | 4830 | 8380 | None |
| Genewiz | TFORF1191 | ZNF573 | XM_017026281, XM_017026280, NM_001172689, | 1281 | 4831 | 8381 | None |
| | | | NM_001172692, ENST00000357309 | | | | |
| Genewiz | TFORF1192 | ZNF573 | NM_152360, ENST00000339503 | 1282 | 4832 | 8382 | None |
| Genewiz | TFORF1193 | ZNF574 | XM_017027149, ENST00000222339 | 1283 | 4833 | 8383 | None |
| Genewiz | TFORF1194 | ZNF575 | XM_011526793, XM_005258783, NM_174945, | 1284 | 4834 | 8384 | None |
| | | | ENST00000314228, ENST00000601282 | | | | |
| Genewiz | TFORF1195 | ZNF576 | NM_024327, NM_001145347, ENST00000391965, | 1285 | 4835 | 8385 | None |
| | | | ENST00000525771, ENST00000533118, ENST00000528387, | | | | |
| | | | ENST00000529930, ENST00000336564 | | | | |
| Genewiz | TFORF1196 | ZNF577 | NM_032679, XM_006723432, XM_006723433, | 1286 | 4836 | 8386 | None |
| | | | XM_011527408, ENST00000301399 | | | | |
| Genewiz | TFORF1197 | ZNF577 | NM_001135590, XM_017027387, ENST00000639636, | 1287 | 4837 | 8387 | None |
| | | | ENST00000451628 | | | | |
| Genewiz | TFORF1198 | ZNF578 | XM_017026302, NM_001099694, ENST00000421239 | 1288 | 4838 | 8388 | None |
| Genewiz | TFORF1199 | ZNF579 | NM_152600, ENST00000325421 | 1289 | 4839 | 8389 | None |
| Genewiz | TFORF1200 | SETDB1 | NM_001243491, XM_017002955, ENST00000368962 | 1290 | 4840 | 8390 | None |
| Genewiz | TFORF1201 | SETDB1 | NM_001145415, ENST00000271640 | 1291 | 4841 | 8391 | None |
| Genewiz | TFORF1202 | SETDB1 | NM_012432, ENST00000368969 | 1292 | 4842 | 8392 | None |
| Genewiz | TFORF1203 | SETDB2 | NM_031915, ENST00000354234 | 1293 | 4843 | 8393 | None |
| Genewiz | TFORF1204 | SETDB2 | NM_001160308, ENST00000317257 | 1294 | 4844 | 8394 | None |
| Genewiz | TFORF1205 | ZNF205 | NM_001042428, NM_003456, NM_001278158, | 1295 | 4845 | 8395 | None |
| | | | XM_005255558, ENST00000382192, ENST00000219091, | | | | |
| | | | ENST00000620094 | | | | |
| Genewiz | TFORF1206 | ZNF202 | NM_001301779, NM_003455, NM_001301780, | 1296 | 4846 | 8396 | None |
| | | | XM_006718901, XM_011542973, XM_011542975, | | | | |
| | | | XM_011542972, XM_005271660, XM_005271661, | | | | |
| | | | XM_005271659, XM_017018268, ENST00000336139, | | | | |
| | | | ENST0530393, ENST00000529691 | | | | |
| Genewiz | TFORF1207 | CASZ1 | NM_001079843, ENST00000377022 | 1297 | 4847 | 8397 | None |
| Genewiz | TFORF1208 | CASZ1 | NM_017766, ENST00000344008 | 1298 | 4848 | 8398 | None |
| Genewiz | TFORF1209 | ZNF200 | NM_001145447, NM_001145448, NM_001145446, | 1299 | 4849 | 8399 | None |
| | | | ENST00000396871, ENST00000396870, ENST00000575948 | | | | |
| Genewiz | TFORF1210 | ZNF200 | NM_198087, ENST00000396868 | 1300 | 4850 | 8400 | None |
| Genewiz | TFORF1211 | EVX1 | NM_001989, ENST00000496902 | 1301 | 4851 | 8401 | None |
| Genewiz | TFORF1212 | ZNF208 | NM_007153, ENST00000397126 | 1302 | 4852 | 8402 | None |
| Genewiz | TFORF1213 | EVX2 | NM_001080458, ENST00000308618 | 1303 | 4853 | 8403 | None |
| Genewiz | TFORF1214 | FOXH1 | NM_003923, ENST00000377317 | 1304 | 4854 | 8404 | None |
| Genewiz | TFORF1215 | NME2 | NM_001198682, ENST00000393183 | 1305 | 4855 | 8405 | None |
| Genewiz | TFORF1216 | NHLH1 | NM_005598, ENST00000302101 | 1306 | 4856 | 8406 | None |
| Genewiz | TFORF1217 | PRRX1 | NM_022716, ENST00000239461 | 1307 | 4857 | 8407 | None |
| Genewiz | TFORF1218 | PRRX1 | NM_006902, ENST00000367760 | 1308 | 4858 | 8408 | None |
| Genewiz | TFORF1219 | PRRX2 | NM_016307, ENST00000372469 | 1309 | 4859 | 8409 | None |
| Genewiz | TFORF1220 | HELT | NM_001300782, ENST00000505610 | 1310 | 4860 | 8410 | None |
| Genewiz | TFORF1221 | HELT | NM_001300781, ENST00000515777 | 1311 | 4861 | 8411 | None |
| Genewiz | TFORF1222 | ZNF483 | NM_001007169, ENST00000358151 | 1312 | 4862 | 8412 | None |
| Genewiz | TFORF1223 | ZNF483 | NM_133464, XM_017014337, XM_017014338, | 1313 | 4863 | 8413 | None |
| | | | XM_011518300, ENST00000309235 | | | | |
| Genewiz | TFORF1224 | AKNA | XM_006717295, ENST00000312033 | 1314 | 4864 | 8414 | None |
| Genewiz | TFORF1225 | AKNA | NM_001317950, NM_030767, XM_005252247, | 1315 | 4865 | 8415 | None |
| | | | XM_005252245, XM_006717294, XM_005252244, | | | | |
| | | | ENST00000307564, ENST00000374088 | | | | |
| Genewiz | TFORF1226 | DUX4 | XM_011531514, ENST00000570263 | 1316 | 4866 | 8416 | None |
| Genewiz | TFORF1227 | DUX4 | NM_001293798, NM_001306068, ENST00000616166, | 1317 | 4867 | 8417 | None |
| | | | ENST00000565211, ENST00000569241 | | | | |
| Genewiz | TFORF1228 | DAXX | NM_001254717, ENST00000414083 | 1318 | 4868 | 8418 | None |
| Genewiz | TFORF1229 | AFF4 | NM_014423, XM_005271963, ENST00000265343 | 1319 | 4869 | 8419 | None |
| Genewiz | TFORF1230 | AFF3 | XM_011511174, XM_011511173, NM_001025108, | 1320 | 4870 | 8420 | None |
| | | | ENST00000409579 | | | | |
| Genewiz | TFORF1231 | AFF3 | XM_005263943, XM_011511176, XM_011511175, | 1321 | 4871 | 8421 | None |
| | | | XM_011511177, NM_002285, ENST00000409236, | | | | |
| | | | ENST00000317233 | | | | |
| Genewiz | TFORF1232 | AFF2 | NM_001169124, ENST00000370457 | 1322 | 4872 | 8422 | None |
| Genewiz | TFORF1233 | AFF2 | NM_001169122, ENST00000342251 | 1323 | 4873 | 8423 | None |
| Genewiz | TFORF1234 | AFF2 | NM_002025, ENST00000370460 | 1324 | 4874 | 8424 | None |
| Genewiz | TFORF1235 | AFF2 | NM_001170628, ENST00000286437 | 1325 | 4875 | 8425 | None |
| Genewiz | TFORF1236 | AFF1 | NM_001166693, XM_011531973, XM_005263007, | 1326 | 4876 | 8426 | None |
| | | | ENST00000395146 | | | | |
| Genewiz | TFORF1237 | AFF1 | NM_005935, ENST00000307808 | 1327 | 4877 | 8427 | None |
| Genewiz | TFORF1238 | HOMEZ | NM_020834, ENST00000357460 | 1328 | 4878 | 8428 | None |
| Genewiz | TFORF1239 | ZNF431 | NM_133473, ENST00000311048 | 1329 | 4879 | 8429 | None |
| Genewiz | TFORF1240 | LBX2 | NM_001009812, ENST00000460508 | 1330 | 4880 | 8430 | None |
| Genewiz | TFORF1241 | LBX2 | NM_001282430, ENST00000377566 | 1331 | 4881 | 8431 | None |
| Genewiz | TFORF1242 | LBX1 | NM_006562, ENST00000370193 | 1332 | 4882 | 8432 | None |
| Genewiz | TFORF1243 | SFPQ | XM_017002053, XM_017002054, XM_005271112, | 1333 | 4883 | 8433 | None |
| | | | XM_005271113, NM_005066, ENST00000357214 | | | | |
| Genewiz | TFORF1244 | PHF20 | XM_017027864, NM_016436, XM_017027867, | 1334 | 4884 | 8434 | None |
| | | | XM_017027866, XM_017027865, ENST00000374012 | | | | |
| Genewiz | TFORF1245 | PKNOX2 | XM_017018110, NM_022062, XM_011542945, | 1335 | 4885 | 8435 | None |
| | | | XM_005271642, XM_011542944, ENST00000298282 | | | | |
| Genewiz | TFORF1246 | ZNF133 | NM_001283004, NM_001283003, XM_017028055, | 1336 | 4886 | 8436 | None |
| | | | XM_017028057, XM_017028056, ENST00000402618 | | | | |
| Genewiz | TFORF1247 | ZNF133 | NM_001282998, NM_001282999, NM_001282997, | 1337 | 4887 | 8437 | None |
| | | | NM_001283001, NM_001283000, XM_011529338, | | | | |
| | | | XM_011529337, XM_017028046, XM017028044, | | | | |
| | | | XM_017028045, XM_011529336, XM_005260820, | | | | |
| | | | XM_011529339, XM_017028048, XM_017028047, | | | | |
| | | | XM_005260819, ENST001790, ENST00000622607, | | | | |
| | | | ENST00000316358 | | | | |
| Genewiz | TFORF1248 | ZNF133 | NM_001283005, ENST00000538547 | 1338 | 4888 | 8438 | None |
| Genewiz | TFORF1249 | ZNF133 | NM_001283002, XM_017028041, XM_017028042, | 1339 | 4889 | 8439 | None |
| | | | ENST00000628216 | | | | |
| Genewiz | TFORF1250 | ZNF133 | NM_001283008, ENST00000630056 | 1340 | 4890 | 8440 | None |
| Genewiz | TFORF1251 | ZNF133 | NM_001283007, ENST00000535822 | 1341 | 4891 | 8441 | None |
| Genewiz | TFORF1252 | TP53 | NM_001276696, ENST00000622645 | 1342 | 4892 | 8442 | None |
| Genewiz | TFORF1253 | TP53 | NM_001126118, NM_001276760, NM_001276761, | 1343 | 4893 | 8443 | None |
| | | | ENST00000610292, ENST00000620739, ENST00000619485 | | | | |
| Genewiz | TFORF1254 | TP53 | NM_001276697, ENST00000619186 | 1344 | 4894 | 8444 | None |
| Genewiz | TFORF1255 | TP53 | NM_001126117, ENST00000504290 | 1345 | 4895 | 8445 | None |
| Genewiz | TFORF1256 | TP53 | NM_001126113, ENST00000455263 | 1346 | 4896 | 8446 | None |
| Genewiz | TFORF1257 | TP53 | NM_000546, NM_001126112, ENST00000269305, | 1347 | 4897 | 8447 | None |
| | | | ENST00000445888 | | | | |
| Genewiz | TFORF1258 | TP53 | NM_001276695, ENST00000610538 | 1348 | 4898 | 8448 | None |
| Genewiz | TFORF1259 | TP53 | NM_001126115, ENST00000504937 | 1349 | 4899 | 8449 | None |
| Genewiz | TFORF1260 | TP53 | NM_001126114, ENST00000617185, ENST00000420246 | 1350 | 4900 | 8450 | None |
| Genewiz | TFORF1261 | TP53 | NM_001126116, ENST00000510385 | 1351 | 4901 | 8451 | None |
| Genewiz | TFORF1262 | TP53 | NM_001276698, ENST00000618944 | 1352 | 4902 | 8452 | None |
| Genewiz | TFORF1263 | TP53 | NM_001276699, ENST00000610623 | 1353 | 4903 | 8453 | None |
| Genewiz | TFORF1264 | ZNF135 | NM_001164530, ENST00000359978 | 1354 | 4904 | 8454 | None |
| Genewiz | TFORF1265 | ZNF135 | XM_006723362, NM_003436, XM_006723363, | 1355 | 4905 | 8455 | None |
| | | | ENST00000511556 | | | | |
| Genewiz | TFORF1266 | ZNF135 | NM_001289401, XM_017027240, ENST00000313434 | 1356 | 4906 | 8456 | None |
| Genewiz | TFORF1267 | ZNF135 | NM_001289402, ENST00000506786 | 1357 | 4907 | 8457 | None |
| Genewiz | TFORF1268 | ZNF135 | NM_007134, ENST00000401053 | 1358 | 4908 | 8458 | None |
| Genewiz | TFORF1269 | HIVEP2 | NM_006734, XM_017010805, ENST00000367603, | 1359 | 4909 | 8459 | None |
| | | | ENST00000012134, ENST00000367604 | | | | |
| Genewiz | TFORF1270 | HLTF | XM_017007079, NM_001318934, ENST00000465259 | 1360 | 4910 | 8460 | None |
| Genewiz | TFORF1271 | HLTF | NM_001318935, NM_003071, NM_139048, | 1361 | 4911 | 8461 | None |
| | | | ENST00000310053, ENST0392912, ENST00000494055 | | | | |
| Genewiz | TFORF1272 | DUXA | NM_001012729, ENST00000554048 | 1362 | 4912 | 8462 | None |
| Genewiz | TFORF1273 | DPF2 | XM_005274149, ENST00000252268 | 1363 | 4913 | 8463 | None |
| Genewiz | TFORF1274 | PKNOX1 | NM_001286258, ENST00000432907 | 1364 | 4914 | 8464 | None |
| Genewiz | TFORF1275 | TPRX1 | NM_198479, ENST00000322175 | 1365 | 4915 | 8465 | None |
| Genewiz | TFORF1276 | ZNF253 | NM_021047, ENST00000589717 | 1366 | 4916 | 8466 | None |
| Genewiz | TFORF1277 | ZNF587 | NM_001204817, ENST00000423137 | 1367 | 4917 | 8467 | None |
| Genewiz | TFORF1278 | ZNF587 | NM_032828, ENST00000339656 | 1368 | 4918 | 8468 | None |
| Genewiz | TFORF1279 | ZNF254 | NM_001278663, XM_011528448, NM_001278678, | 1369 | 4919 | 8469 | None |
| | | | ENST00000616028 | | | | |
| Genewiz | TFORF1280 | ZNF254 | XM_017027518, XM_017027519, NM_001278664, | 1370 | 4920 | 8470 | None |
| | | | ENST00000611359 | | | | |
| Genewiz | TFORF1281 | ZNF254 | NM_001278661, NM_001278677, XM_011528443, | 1371 | 4921 | 8471 | None |
| | | | XM_017027515, XM_011528444, XM_017027514, | | | | |
| | | | NM_001278662, XM_017027516, ENST00000613065 | | | | |
| Genewiz | TFORF1282 | SALL4 | NM_001318031, ENST00000395997 | 1372 | 4922 | 8472 | None |
| Genewiz | TFORF1283 | SALL1 | NM_001127892, ENST00000440970 | 1373 | 4923 | 8473 | None |
| Genewiz | TFORF1284 | SALL1 | NM_002968, XM_011523254, XM_006721241, | 1374 | 4924 | 8474 | None |
| | | | ENST00000251020 | | | | |
| Genewiz | TFORF1285 | ZNF257 | NM_033468, ENST00000594947 | 1375 | 4925 | 8475 | None |
| Genewiz | TFORF1286 | SALL3 | NM_171999, ENST00000537592 | 1376 | 4926 | 8476 | None |
| Genewiz | TFORF1287 | SALL2 | NM_005407, ENST00000614342 | 1377 | 4927 | 8477 | None |
| Genewiz | TFORF1288 | SALL2 | NM_001291447, ENST00000450879 | 1378 | 4928 | 8478 | None |
| Genewiz | TFORF1289 | GMEB2 | NM_012384, XM_005260202, ENST00000266068, | 1379 | 4929 | 8479 | None |
| | | | ENST00000370077 | | | | |
| Genewiz | TFORF1290 | GMEB1 | NM_006582, XM_011540519, XM_011540518, | 1380 | 4930 | 8480 | None |
| | | | XM_017000087, ENST00000294409 | | | | |
| Genewiz | TFORF1291 | SP140L | NM_001308163, ENST00000396563 | 1381 | 4931 | 8481 | None |
| Genewiz | TFORF1292 | SP140L | NM_138402, ENST00000415673 | 1382 | 4932 | 8482 | None |
| Genewiz | TFORF1293 | SP140L | NM_001308162, ENST00000243810 | 1383 | 4933 | 8483 | None |
| Genewiz | TFORF1294 | ZFP64 | NM_022088, ENST00000346617 | 1384 | 4934 | 8484 | None |
| Genewiz | TFORF1295 | ZFP64 | NM_001319146, ENST00000371523 | 1385 | 4935 | 8485 | None |
| Genewiz | TFORF1296 | ZFP64 | NM_199427, ENST00000361387 | 1386 | 4936 | 8486 | None |
| Genewiz | TFORF1297 | ZFP64 | NM_199426, ENST00000371515 | 1387 | 4937 | 8487 | None |
| Genewiz | TFORF1298 | ZFP64 | NM_018197, ENST00000216923 | 1388 | 4938 | 8488 | None |
| Genewiz | TFORF1299 | MAEL | XM_017002602, NM_001286378, XM_017002603, | 1389 | 4939 | 8489 | None |
| | | | ENST00000622874 | | | | |
| Genewiz | TFORF1300 | MAEL | NM_001286377, ENST00000367870 | 1390 | 4940 | 8490 | None |
| Genewiz | TFORF1301 | HMG20A | NM_001304504, NM_018200, XM_011521158, | 1391 | 4941 | 8491 | None |
| | | | ENST00000336216, ENST00000381714 | | | | |
| Genewiz | TFORF1302 | ZMIZ2 | XM_005249872, NM_174929, ENST00000265346 | 1392 | 4942 | 8492 | None |
| Genewiz | TFORF1303 | ZMIZ2 | XM_005249873, ENST00000433667 | 1393 | 4943 | 8493 | None |
| Genewiz | TFORF1304 | ZMIZ2 | XM_005249869, NM_031449, ENST00000309315, | 1394 | 4944 | 8494 | None |
| | | | ENST00000441627 | | | | |
| Genewiz | TFORF1305 | ZMIZ1 | NM_020338, ENST00000334512 | 1395 | 4945 | 8495 | None |
| Genewiz | TFORF1306 | ZSCAN9 | NM_001199479, ENST00000425468 | 1396 | 4946 | 8496 | None |
| Genewiz | TFORF1307 | JARID2 | NM_004973, ENST00000341776 | 1397 | 4947 | 8497 | None |
| Genewiz | TFORF1308 | JARID2 | XM_017010834, NM_001267040, XM_005249089, | 1398 | 4948 | 8498 | None |
| | | | XM_017010835, ENST00000397311 | | | | |
| Genewiz | TFORF1309 | TTF1 | NM_007344, XM_006717273, ENST00000334270 | 1399 | 4949 | 8499 | None |
| Genewiz | TFORF1310 | TTF1 | NM_001205296, ENST00000612514 | 1400 | 4950 | 8500 | None |
| Genewiz | TFORF1311 | RBMS1 | NM_016836, ENST00000348849 | 1401 | 4951 | 8501 | None |
| Genewiz | TFORF1312 | AEBP2 | NM_001267043, ENST00000360995 | 1402 | 4952 | 8502 | None |
| Genewiz | TFORF1313 | AEBP2 | NM_153207, ENST00000266508 | 1403 | 4953 | 8503 | None |
| Genewiz | TFORF1314 | AEBP2 | NM_001114176, ENST00000398864 | 1404 | 4954 | 8504 | None |
| Genewiz | TFORF1315 | ZNF813 | NM_001004301, ENST00000396403 | 1405 | 4955 | 8505 | None |
| Genewiz | TFORF1316 | RB1CC1 | NM_001083617, XM_017014107, ENST00000435644 | 1406 | 4956 | 8506 | None |
| Genewiz | TFORF1317 | RB1CC1 | NM_014781, XM_011517643, ENST00000025008 | 1407 | 4957 | 8507 | None |
| Genewiz | TFORF1318 | MKX | NM_173576, NM_001242702, XM_017016106, | 1408 | 4958 | 8508 | None |
| | | | XM_017016105, ENST00000419761, ENST00000375790 | | | | |
| Genewiz | TFORF1319 | KLF13 | NM_015995, ENST00000307145 | 1409 | 4959 | 8509 | None |
| Genewiz | TFORF1320 | KLF11 | NM_001177716, NM_001177718, ENST00000540845, | 1410 | 4960 | 8510 | None |
| | | | ENST00000535335 | | | | |
| Genewiz | TFORF1321 | KLF11 | NM_003597, ENST00000305883 | 1411 | 4961 | 8511 | None |
| Genewiz | TFORF1322 | KLF10 | NM_005655, ENST00000285407 | 1412 | 4962 | 8512 | None |
| Genewiz | TFORF1323 | KLF10 | NM_001032282, ENST00000395884 | 1413 | 4963 | 8513 | None |
| Genewiz | TFORF1324 | KLF17 | NM_173484, ENST00000372299 | 1414 | 4964 | 8514 | None |
| Genewiz | TFORF1325 | KLF16 | NM_031918, ENST00000541015, ENST00000250916, | 1415 | 4965 | 8515 | None |
| | | | ENST00000617223 | | | | |
| Genewiz | TFORF1326 | KLF15 | NM_014079, XM_005247400, ENST00000296233 | 1416 | 4966 | 8516 | None |
| Genewiz | TFORF1327 | KLF14 | NM_138693, ENST00000583337 | 1417 | 4967 | 8517 | None |
| Genewiz | TFORF1328 | TFAP2A | NM_001042425, ENST00000319516 | 1418 | 4968 | 8518 | None |
| Genewiz | TFORF1329 | TFAP2A | NM_003220, ENST00000482890 | 1419 | 4969 | 8519 | None |
| Genewiz | TFORF1330 | TFAP2C | NM_003222, ENST00000201031 | 1420 | 4970 | 8520 | None |
| Genewiz | TFORF1331 | TFAP2B | NM_003221, ENST00000393655 | 1421 | 4971 | 8521 | None |
| Genewiz | TFORF1332 | TFAP2E | NM_178548, ENST00000373235 | 1422 | 4972 | 8522 | None |
| Genewiz | TFORF1333 | TFAP2D | NM_172238, ENST00000008391 | 1423 | 4973 | 8523 | None |
| Genewiz | TFORF1334 | POU3F3 | NM_006236, ENST00000361360 | 1424 | 4974 | 8524 | None |
| Genewiz | TFORF1335 | POU3F2 | NM_005604, ENST00000328345 | 1425 | 4975 | 8525 | None |
| Genewiz | TFORF1336 | POU3F1 | NM_002699, ENST00000373012 | 1426 | 4976 | 8526 | None |
| Genewiz | TFORF1337 | ZBTB7A | NM_001317990, NM_015898, XM_005259571, | 1427 | 4977 | 8527 | None |
| | | | ENST00000322357, ENST00000601588 | | | | |
| Genewiz | TFORF1338 | ZBTB7C | XM_017025609, XM_011525870, XM_005258229, | 1428 | 4978 | 8528 | None |
| | | | NM_001318841, NM_001039360, XM_011525871, | | | | |
| | | | ENST00000535628, ENST00000586438, ENST00000588982, | | | | |
| | | | ENST00000590800 | | | | |
| Genewiz | TFORF1339 | POU3F4 | NM_000307, ENST00000373200 | 1429 | 4979 | 8529 | None |
| Genewiz | TFORF1340 | POU5F1 | NM_001285986, ENST00000441888, ENST00000471529, | 1430 | 4980 | 8530 | None |
| | | | ENST00000512818, ENST00000513407 | | | | |
| Genewiz | TFORF1341 | POU5F1 | NM_203289, NM_001173531, ENST00000606567, | 1431 | 4981 | 8531 | None |
| | | | ENST00000620031 | | | | |
| Genewiz | TFORF1342 | POU5F2 | NM_153216, ENST00000606183 | 1432 | 4982 | 8532 | None |
| Genewiz | TFORF1343 | FEZF1 | XM_011516202, NM_001160264, ENST00000427185 | 1433 | 4983 | 8533 | None |
| Genewiz | TFORF1344 | FEZF2 | NM_018008, ENST00000486811, ENST00000283268, | 1434 | 4984 | 8534 | None |
| | | | ENST00000475839 | | | | |
| Genewiz | TFORF1345 | RPA1 | NM_002945, ENST00000254719 | 1435 | 4985 | 8535 | None |
| Genewiz | TFORF1346 | ZNF541 | NM_001277075, XM_005259311, ENST00000391901 | 1436 | 4986 | 8536 | None |
| Genewiz | TFORF1347 | RPA3 | NM_002947, ENST00000223129, ENST00000396682 | 1437 | 4987 | 8537 | None |
| Genewiz | TFORF1348 | RPA2 | NM_001297558, ENST00000373909 | 1438 | 4988 | 8538 | None |
| Genewiz | TFORF1349 | POGZ | NM_207171, XM_017000749, XM_005245006, | 1439 | 4989 | 8539 | None |
| | | | XM_017000748, XM_005245005, ENST00000392723 | | | | |
| Genewiz | TFORF1350 | POGZ | NM_001194937, XM_017000745, XM_017000746, | 1440 | 4990 | 8540 | None |
| | | | ENST00000409503 | | | | |
| Genewiz | TFORF1351 | POGZ | NM_145796, ENST00000368863 | 1441 | 4991 | 8541 | None |
| Genewiz | TFORF1352 | POGZ | NM_015100, XM_005244999, XM_005245000, | 1442 | 4992 | 8542 | None |
| | | | XM_005245001, ENST00000271715 | | | | |
| Genewiz | TFORF1353 | POGZ | NM_001194938, ENST00000531094 | 1443 | 4993 | 8543 | None |
| Genewiz | TFORF1354 | ILF3 | NM_017620, XM_017026763, XM_011527984, | 1444 | 4994 | 8544 | None |
| | | | ENST00000449870, ENST00000588657 | | | | |
| Genewiz | TFORF1355 | ILF3 | NM_012218, ENST00000590261 | 1445 | 4995 | 8545 | None |
| Genewiz | TFORF1356 | ILF3 | NM_004516, ENST00000589998 | 1446 | 4996 | 8546 | None |
| Genewiz | TFORF1357 | ILF3 | NM_153464, ENST00000250241 | 1447 | 4997 | 8547 | None |
| Genewiz | TFORF1358 | SP100 | NM_003113, ENST00000264052 | 1448 | 4998 | 8548 | None |
| Genewiz | TFORF1359 | SP100 | NM_001206701, ENST00000409112 | 1449 | 4999 | 8549 | None |
| Genewiz | TFORF1360 | SP100 | NM_001206703, ENST00000427101 | 1450 | 5000 | 8550 | None |
| Genewiz | TFORF1361 | SP100 | NM_001080391, ENST00000340126 | 1451 | 5001 | 8551 | None |
| Genewiz | TFORF1362 | SP100 | NM_001206704, ENST00000409897 | 1452 | 5002 | 8552 | None |
| Genewiz | TFORF1363 | ELOF1 | XM_017027356, ENST00000587806 | 1453 | 5003 | 8553 | None |
| Genewiz | TFORF1364 | ELOF1 | XM_017027357, ENST00000591674 | 1454 | 5004 | 8554 | None |
| Genewiz | TFORF1365 | ISL1 | NM_002202, ENST00000230658 | 1455 | 5005 | 8555 | None |
| Genewiz | TFORF1366 | ISL2 | NM_145805, ENST00000290759 | 1456 | 5006 | 8556 | None |
| Genewiz | TFORF1367 | RREB1 | NM_001003699, XM_006715157, ENST00000379938 | 1457 | 5007 | 8557 | None |
| Genewiz | TFORF1368 | RREB1 | NM_001003700, ENST00000334984 | 1458 | 5008 | 8558 | None |
| Genewiz | TFORF1369 | RREB1 | NM_001168344, NM_001003698, ENST00000379933, | 1459 | 5009 | 8559 | None |
| | | | ENST00000349384 | | | | |
| Genewiz | TFORF1370 | ZNF141 | NM_003441, ENST00000240499 | 1460 | 5010 | 8560 | None |
| Genewiz | TFORF1371 | ZNF140 | XM_017019925, NM_001300776, NM_001300778, | 1461 | 5011 | 8561 | None |
| | | | XM_011534840, XM_017019924, ENST00000544426 | | | | |
| Genewiz | TFORF1372 | ZNF143 | NM_001282657, ENST00000396597 | 1462 | 5012 | 8562 | None |
| Genewiz | TFORF1373 | ZNF143 | NM_001282656, XM_017018254, XM_017018255, | 1463 | 5013 | 8563 | None |
| | | | ENST00000396604, ENST00000530463 | | | | |
| Genewiz | TFORF1374 | ZNF143 | NM_003442, XM_011520349, ENST00000396602 | 1464 | 5014 | 8564 | None |
| Genewiz | TFORF1375 | ZNF142 | XM_017004872, NM_001105537, XM_011511789, | 1465 | 5015 | 8565 | None |
| | | | ENST00000449707, ENST00000411696 | | | | |
| Genewiz | TFORF1376 | ZNF146 | NM_001099639, NM_001099638, NM_007145, | 1466 | 5016 | 8566 | None |
| | | | XM_005259214, XM_017027247, XM_017027245, | | | | |
| | | | XM_017027246, XM_017027244, ENST0456324, | | | | |
| | | | ENST00000443387 | | | | |
| Genewiz | TFORF1377 | ZNF148 | NM_021964, ENST00000360647, ENST00000484491, | 1467 | 5017 | 8567 | None |
| | | | ENST00000492394, ENST00000485866 | | | | |
| Genewiz | TFORF1378 | ZNF783 | NM_001195220, ENST00000434415 | 1468 | 5018 | 8568 | None |
| Genewiz | TFORF1379 | ZNF782 | XM_011518315, XM_005251742, XM_011518318, | 1469 | 5019 | 8569 | None |
| | | | NM_001001662, ENST00000481138, ENST00000535338 | | | | |
| Genewiz | TFORF1380 | ZNF787 | NM_001002836, XM_011526445, ENST00000610935 | 1470 | 5020 | 8570 | None |
| Genewiz | TFORF1381 | ZNF789 | NM_001013258, ENST00000379724 | 1471 | 5021 | 8571 | None |
| Genewiz | TFORF1382 | ZNF789 | XM_017012018, NM_213603, ENST00000331410 | 1472 | 5022 | 8572 | None |
| Genewiz | TFORF1383 | ZNF788 | ENST00000430298 | 1473 | 5023 | 8573 | None |
| Genewiz | TFORF1384 | ZNF788 | ENST00000596883 | 1474 | 5024 | 8574 | None |
| Genewiz | TFORF1385 | TMF | NM_007114, ENST00000398559 | 1475 | 5025 | 8575 | None |
| Genewiz | TFORF1386 | ZNF362 | NM_152493, XM_017000415, ENST00000539719, | 1476 | 5026 | 8576 | None |
| | | | ENST00000373428 | | | | |
| Genewiz | TFORF1387 | ZNF367 | NM_153695, ENST00000375256 | 1477 | 5027 | 8577 | None |
| Genewiz | TFORF1388 | PMS1 | NM_001128144, XM_017004348, ENST00000447232 | 1478 | 5028 | 8578 | None |
| Genewiz | TFORF1389 | PMS1 | NM_001321051, ENST00000374826 | 1479 | 5029 | 8579 | None |
| Genewiz | TFORF1390 | PMS1 | NM_001289409, NM_001289408, XM_011511356, | 1480 | 5030 | 8580 | None |
| | | | ENST00000432292, ENST00000624204 | | | | |
| Genewiz | TFORF1391 | PMS1 | NM_001321048, NM_000534, NM_001321045, | 1481 | 5031 | 8581 | None |
| | | | NM_001321047, ENST00000441310 | | | | |
| Genewiz | TFORF1392 | PMS1 | NM_001128143, XM_017004344, ENST00000409823 | 1482 | 5032 | 8582 | None |
| Genewiz | TFORF1393 | PMS1 | NM_001321049, ENST00000409985 | 1483 | 5033 | 8583 | None |
| Genewiz | TFORF1394 | ZNF569 | XM_017026381, XM_017026380, XM_017026379, | 1484 | 5034 | 8584 | None |
| | | | XM_017026382, ENST00000392150 | | | | |
| Genewiz | TFORF1395 | ZNF569 | XM_011526539, XM_017026376, XM_017026377, | 1485 | 5035 | 8585 | None |
| | | | NM_152484, ENST00000392149, ENST00000316950 | | | | |
| Genewiz | TFORF1396 | ZNF568 | NM_198539, ENST00000333987, ENST00000619231 | 1486 | 5036 | 8586 | None |
| Genewiz | TFORF1397 | ZNF568 | NM_001204837, NM_001204836, XM_017026773, | 1487 | 5037 | 8587 | None |
| | | | XM_017026774, ENST00000415168 | | | | |
| Genewiz | TFORF1398 | ZNF568 | NM_001204839, XM_017026775, ENST00000455427 | 1488 | 5038 | 8588 | None |
| Genewiz | TFORF1399 | ZNF568 | NM_001204838, XM_017026772, ENST00000617745 | 1489 | 5039 | 8589 | None |
| Genewiz | TFORF1400 | ZNF567 | XM_017026420, NM_001322911, NM_152603, | 1490 | 5040 | 8590 | None |
| | | | NM_001322912, XM_017026421, XM_011526584, | | | | |
| | | | ENST00000360729, ENST00000585696 | | | | |
| Genewiz | TFORF1401 | ZNF567 | XM_017026417, NM_001322916, NM_001322915, | 1491 | 5041 | 8591 | None |
| | | | NM_001322919, NM_001322917, NM_001322918, | | | | |
| | | | NM_001322914, NM_001300979, NM_001322920, | | | | |
| | | | NM_001322913, XM_017026418, ENST00000536254 | | | | |
| Genewiz | TFORF1402 | ZNF567 | XM_011526585, ENST00000588311 | 1492 | 5042 | 8592 | None |
| Genewiz | TFORF1403 | ZNF566 | NM_001300970, XM_017027400, XM_005259356, | 1493 | 5043 | 8593 | None |
| | | | XM_017027399, XM_011527428, ENST00000493391 | | | | |
| Genewiz | TFORF1404 | ZNF566 | NM_001145343, XM_006723447, ENST00000392170 | 1494 | 5044 | 8594 | None |
| Genewiz | TFORF1405 | ZNF566 | NM_032838, NM_001145345, NM_001145344, | 1495 | 5045 | 8595 | None |
| | | | ENST00000434377, ENST00000424129 | | | | |
| Genewiz | TFORF1406 | ZNF565 | NM_152477, NM_001042474, XM_011526514, | 1496 | 5046 | 8596 | None |
| | | | XM_011526512, XM_017026341, ENST00000304116, | | | | |
| | | | ENST00000392173 | | | | |
| Genewiz | TFORF1407 | ZNF559 | NM_001202409, ENST00000592896 | 1497 | 5047 | 8597 | None |
| Genewiz | TFORF1408 | ZNF559 | NM_001202406, ENST00000587557 | 1498 | 5048 | 8598 | None |
| Genewiz | TFORF1409 | ZNF559 | NM_001202410, NM_001202411, NM_001202412, | 1499 | 5049 | 8599 | None |
| | | | ENST00000585352, ENST00000317221 | | | | |
| Genewiz | TFORF1410 | RNF2 | NM_007212, XM_011509852, XM_011509851, | 1500 | 5050 | 8600 | None |
| | | | ENST00000367510 | | | | |
| Genewiz | TFORF1411 | ZNF562 | XM_017026898, NM_001300885, ENST00000590155 | 1501 | 5051 | 8601 | None |
| Genewiz | TFORF1412 | ZNF562 | NM_017656, ENST00000293648 | 1502 | 5052 | 8602 | None |
| Genewiz | TFORF1413 | ZNF561 | XM_017027481, XM_017027479, XM_017027480, | 1503 | 5053 | 8603 | None |
| | | | ENST00000424629 | | | | |
| Genewiz | TFORF1414 | ZNF561 | NM_152289, XM_005260150, ENST00000302851 | 1504 | 5054 | 8604 | None |
| Genewiz | TFORF1415 | ZNF560 | XM_011527696, XM_017026327, XM_017026329, | 1505 | 5055 | 8605 | None |
| | | | XM_017026328, XM_011527697, NM_152476, | | | | |
| | | | ENST00000301480 | | | | |
| Genewiz | TFORF1416 | ZNF215 | NM_013250, XM_006718311, ENST00000278319, | 1506 | 5056 | 8606 | None |
| | | | ENST00000414517 | | | | |
| Genewiz | TFORF1417 | ZNF214 | XM_005253128, XM_006718308, NM_013249, | 1507 | 5057 | 8607 | None |
| | | | ENST00000278314, ENST00000536068 | | | | |
| Genewiz | TFORF1418 | ZNF217 | NM_006526, XM_017028059, ENST00000371471, | 1508 | 5058 | 8608 | None |
| | | | ENST00000302342 | | | | |
| Genewiz | TFORF1419 | VDR | NM_001017536, ENST00000550325 | 1509 | 5059 | 8609 | None |
| Genewiz | TFORF1420 | ZNF211 | NM_006385, ENST00000240731 | 1510 | 5060 | 8610 | None |
| Genewiz | TFORF1421 | ZNF211 | NM_001265599, ENST00000254182 | 1511 | 5061 | 8611 | None |
| Genewiz | TFORF1422 | ZNF211 | NM_001265600, ENST00000391703 | 1512 | 5062 | 8612 | None |
| Genewiz | TFORF1423 | ZNF211 | NM_001265597, ENST00000299871 | 1513 | 5063 | 8613 | None |
| Genewiz | TFORF1424 | ZNF211 | NM_001265598, ENST00000541801 | 1514 | 5064 | 8614 | None |
| Genewiz | TFORF1425 | ZNF211 | NM_198855, ENST00000347302 | 1515 | 5065 | 8615 | None |
| Genewiz | TFORF1426 | FOXK2 | NM_004514, ENST00000335255 | 1516 | 5066 | 8616 | None |
| Genewiz | TFORF1427 | FOXK1 | NM_001037165, ENST00000328914 | 1517 | 5067 | 8617 | None |
| Genewiz | TFORF1428 | NR3C2 | NM_001166104, ENST00000512865 | 1518 | 5068 | 8618 | None |
| Genewiz | TFORF1429 | NR3C2 | XM_011531975, XM_011531976, XM_011531977, | 1519 | 5069 | 8619 | None |
| | | | ENST00000625323, ENST00000511528 | | | | |
| Genewiz | TFORF1430 | NFKBIB | NM_001243116, ENST00000392079 | 1520 | 5070 | 8620 | None |
| Genewiz | TFORF1431 | ZNF219 | NM_001102454, XM_006720164, NM_016423, | 1521 | 5071 | 8621 | None |
| | | | NM_001101672, XM_017021354, XM_006720163, | | | | |
| | | | XM_017021355, ENST00000360947, ENST00000451119, | | | | |
| | | | ENST00000421093 | | | | |
| Genewiz | TFORF1432 | NR3C1 | NM_001024094, XM_005268420, XM_005268423, | 1522 | 5072 | 8622 | None |
| | | | XM_005268419, XM_005268422, ENST00000504572, | | | | |
| | | | ENST00000394466, ENST00000231509 | | | | |
| Genewiz | TFORF1433 | NR3C1 | NM_001020825, ENST00000415690 | 1523 | 5073 | 8623 | None |
| Genewiz | TFORF1434 | NFKBID | XM_011527419, ENST00000606253 | 1524 | 5074 | 8624 | None |
| Genewiz | TFORF1435 | NFKBIE | NM_004556, ENST00000275015 | 1525 | 5075 | 8625 | None |
| Genewiz | TFORF1436 | NFKBIZ | NM_031419, ENST00000326172 | 1526 | 5076 | 8626 | None |
| Genewiz | TFORF1437 | NFKBIZ | NM_001005474, ENST00000394054 | 1527 | 5077 | 8627 | None |
| Genewiz | TFORF1438 | EP400 | NM_015409, ENST00000389561, ENST00000389562 | 1528 | 5078 | 8628 | None |
| Genewiz | TFORF1439 | ZNF846 | XM_011527717, XM_005259772, NM_001077624, | 1529 | 5079 | 8629 | None |
| | | | ENST00000397902 | | | | |
| Genewiz | TFORF1440 | ARID4A | NM_002892, ENST00000355431 | 1530 | 5080 | 8630 | None |
| Genewiz | TFORF1441 | ARID4A | NM_023001, ENST00000348476, ENST00000431317 | 1531 | 5081 | 8631 | None |
| Genewiz | TFORF1442 | ARID4A | NM_023000, ENST00000395168 | 1532 | 5082 | 8632 | None |
| Genewiz | TFORF1443 | ARID4B | NM_031371, ENST00000349213 | 1533 | 5083 | 8633 | None |
| Genewiz | TFORF1444 | ARID4B | NM_001206794, NM_016374, XM_011544212, | 1534 | 5084 | 8634 | None |
| | | | ENST00000366603, ENST00000264183 | | | | |
| Genewiz | TFORF1445 | GTF2H3 | NM_001271868, XM_017019228, ENST00000618160 | 1535 | 5085 | 8635 | None |
| Genewiz | TFORF1446 | GTF2H3 | NM_001271867, ENST00000228955 | 1536 | 5086 | 8636 | None |
| Genewiz | TFORF1447 | NR5A2 | XM_011509382, XM_005245062, NM_001276464, | 1537 | 5087 | 8637 | None |
| | | | ENST00000544748 | | | | |
| Genewiz | TFORF1448 | NR5A2 | NM_205860, ENST00000367362 | 1538 | 5088 | 8638 | None |
| Genewiz | TFORF1449 | DLX4 | XM_017024291, NM_001934, ENST00000411890 | 1539 | 5089 | 8639 | None |
| Genewiz | TFORF1450 | DLX6 | NM_005222, ENST00000518156 | 1540 | 5090 | 8640 | None |
| Genewiz | TFORF1451 | DLX1 | NM_178120, ENST00000361725 | 1541 | 5091 | 8641 | None |
| Genewiz | TFORF1452 | DLX1 | NM_001038493, ENST00000341900 | 1542 | 5092 | 8642 | None |
| Genewiz | TFORF1453 | DLX2 | NM_004405, ENST00000234198 | 1543 | 5093 | 8643 | None |
| Genewiz | TFORF1454 | TFEC | NM_001018058, ENST00000320239 | 1544 | 5094 | 8644 | None |
| Genewiz | TFORF1455 | TFEC | NM_012252, XM_017011875, ENST00000265440 | 1545 | 5095 | 8645 | None |
| Genewiz | TFORF1456 | TFEC | NM_001244583, ENST00000457268 | 1546 | 5096 | 8646 | None |
| Genewiz | TFORF1457 | TFEB | NM_001271943, ENST00000420312 | 1547 | 5097 | 8647 | None |
| Genewiz | TFORF1458 | TFEB | NM_001167827, ENST00000358871 | 1548 | 5098 | 8648 | None |
| Genewiz | TFORF1459 | LMX1A | NM_001174069, NM_177398, ENST00000342310, | 1549 | 5099 | 8649 | None |
| | | | ENST00000294816, ENST00000367893 | | | | |
| Genewiz | TFORF1460 | LMX1B | NM_001174147, ENST00000373474 | 1550 | 5100 | 8650 | None |
| Genewiz | TFORF1461 | LMX1B | NM_001174146, ENST00000355497 | 1551 | 5101 | 8651 | None |
| Genewiz | TFORF1462 | LMX1B | NM_002316, ENST00000526117 | 1552 | 5102 | 8652 | None |
| Genewiz | TFORF1463 | VAV1 | NM_001258206, ENST00000304076 | 1553 | 5103 | 8653 | None |
| Genewiz | TFORF1464 | VAV1 | NM_001258207, ENST00000596764 | 1554 | 5104 | 8654 | None |
| Genewiz | TFORF1465 | VAV1 | NM_005428, ENST00000602142 | 1555 | 5105 | 8655 | None |
| Genewiz | TFORF1466 | HOXD12 | NM_021193, ENST00000406506 | 1556 | 5106 | 8656 | None |
| Genewiz | TFORF1467 | HOXD13 | NM_000523, ENST00000392539 | 1557 | 5107 | 8657 | None |
| Genewiz | TFORF1468 | HOXD11 | NM_021192, ENST00000249504 | 1558 | 5108 | 8658 | None |
| Genewiz | TFORF1469 | TOX3 | NM_001080430, ENST00000219746 | 1559 | 5109 | 8659 | None |
| Genewiz | TFORF1470 | TOX3 | NM_001146188, ENST00000407228 | 1560 | 5110 | 8660 | None |
| Genewiz | TFORF1471 | TOX2 | NM_001098797, ENST00000341197 | 1561 | 5111 | 8661 | None |
| Genewiz | TFORF1472 | TOX2 | NM_001098798, ENST00000358131 | 1562 | 5112 | 8662 | None |
| Genewiz | TFORF1473 | LIN54 | NM_001115008, NM_001288997, NM_001115007, | 1563 | 5113 | 8663 | None |
| | | | ENST00000442461, ENST00000446851, ENST00000510557 | | | | |
| Genewiz | TFORF1474 | LIN54 | NM_194282, XM_006714081, XM_005262750, | 1564 | 5114 | 8664 | None |
| | | | ENST00000340417, ENST00000505397 | | | | |
| Genewiz | TFORF1475 | LIN54 | NM_001288996, XM_017007728, ENST00000506560 | 1565 | 5115 | 8665 | None |
| Genewiz | TFORF1476 | VAX1 | NM_199131, ENST00000277905 | 1566 | 5116 | 8666 | None |
| Genewiz | TFORF1477 | VAX1 | NM_001112704, ENST00000369206 | 1567 | 5117 | 8667 | None |
| Genewiz | TFORF1478 | TP63 | XM_017007387, ENST00000456148 | 1568 | 5118 | 8668 | None |
| Genewiz | TFORF1479 | TP63 | NM_001114980, ENST00000354600 | 1569 | 5119 | 8669 | None |
| Genewiz | TFORF1480 | TP63 | NM_001114978, ENST00000392460 | 1570 | 5120 | 8670 | None |
| Genewiz | TFORF1481 | TP63 | NM_001114982, ENST00000437221 | 1571 | 5121 | 8671 | None |
| Genewiz | TFORF1482 | TP63 | NM_001114979, ENST00000418709 | 1572 | 5122 | 8672 | None |
| Genewiz | TFORF1483 | TP63 | NM_001114981, ENST00000392463 | 1573 | 5123 | 8673 | None |
| Genewiz | TFORF1484 | TP63 | XM_005247843, ENST00000440651 | 1574 | 5124 | 8674 | None |
| Genewiz | TFORF1485 | NANOG | NM_024865, ENST00000229307 | 1575 | 5125 | 8675 | None |
| Genewiz | TFORF1486 | NANOG | NM_001297698, ENST00000526286 | 1576 | 5126 | 8676 | None |
| Genewiz | TFORF1487 | NR6A1 | XM_005251917, ENST00000344523 | 1577 | 5127 | 8677 | None |
| Genewiz | TFORF1488 | NR6A1 | NM_001278546, ENST00000373584 | 1578 | 5128 | 8678 | None |
| Genewiz | TFORF1489 | NR6A1 | NM_033334, ENST00000487099 | 1579 | 5129 | 8679 | None |
| Genewiz | TFORF1490 | NR6A1 | NM_001489, ENST00000416460 | 1580 | 5130 | 8680 | None |
| Genewiz | TFORF1491 | ZNF57 | NM_001319083, XM_011527682, ENST00000523428 | 1581 | 5131 | 8681 | None |
| Genewiz | TFORF1492 | FOSL1 | NM_001300855, ENST00000532401 | 1582 | 5132 | 8682 | None |
| Genewiz | TFORF1493 | FOSL1 | NM_001300844, ENST00000531493 | 1583 | 5133 | 8683 | None |
| Genewiz | TFORF1494 | FOSL1 | NM_001300857, ENST00000448083 | 1584 | 5134 | 8684 | None |
| Genewiz | TFORF1495 | FOXN3 | NM_001085471, ENST00000345097, ENST00000261302 | 1585 | 5135 | 8685 | None |
| Genewiz | TFORF1496 | HHEX | NM_002729, ENST00000282728 | 1586 | 5136 | 8686 | None |
| Genewiz | TFORF1497 | LYL1 | NM_005583, ENST00000264824 | 1587 | 5137 | 8687 | None |
| Genewiz | TFORF1498 | RBL1 | NM_002895, ENST00000373664 | 1588 | 5138 | 8688 | None |
| Genewiz | TFORF1499 | RBL1 | NM_183404, ENST00000344359 | 1589 | 5139 | 8689 | None |
| Genewiz | TFORF1500 | RBL2 | NM_001323608, NM_005611, ENST00000262133 | 1590 | 5140 | 8690 | None |
| Genewiz | TFORF1501 | STOX2 | NM_020225, ENST00000308497 | 1591 | 5141 | 8691 | None |
| Genewiz | TFORF1502 | GABPB1 | NM_016654, XM_017022053, ENST00000380877 | 1592 | 5142 | 8692 | None |
| Genewiz | TFORF1503 | GABPB1 | NM_181427, NM_016655, ENST00000396464, | 1593 | 5143 | 8693 | None |
| | | | ENST00000359031 | | | | |
| Genewiz | TFORF1504 | GABPB1 | NM_002041, ENST00000429662 | 1594 | 5144 | 8694 | None |
| Genewiz | TFORF1505 | ZNF696 | NM_030895, ENST00000330143 | 1595 | 5145 | 8695 | None |
| Genewiz | TFORF1506 | HEY2 | XM_017010629, XM_017010628, XM_017010627, | 1596 | 5146 | 8696 | None |
| | | | ENST00000368365 | | | | |
| Genewiz | TFORF1507 | HEY1 | NM_001282851, ENST00000523976 | 1597 | 5147 | 8697 | None |
| Genewiz | TFORF1508 | MTF1 | NM_005955, XM_011541491, ENST00000373036 | 1598 | 5148 | 8698 | None |
| Genewiz | TFORF1509 | STAT5B | NM_012448, ENST00000293328 | 1599 | 5149 | 8699 | None |
| Genewiz | TFORF1510 | STAT5A | NM_001288719, ENST00000546010 | 1600 | 5150 | 8700 | None |
| Genewiz | TFORF1511 | STAT5A | NM_001288720, ENST00000588868 | 1601 | 5151 | 8701 | None |
| Genewiz | TFORF1512 | NKX3-2 | NM_001189, ENST00000382438 | 1602 | 5152 | 8702 | None |
| Genewiz | TFORF1513 | NKX3-1 | NM_006167, ENST00000380871 | 1603 | 5153 | 8703 | None |
| Genewiz | TFORF1514 | NKX3-1 | NM_001256339, ENST00000523261 | 1604 | 5154 | 8704 | None |
| Genewiz | TFORF1515 | NANOGNB | NM_001145465, ENST00000382119 | 1605 | 5155 | 8705 | None |
| Genewiz | TFORF1516 | ZNF737 | NM_001159293, ENST00000427401 | 1606 | 5156 | 8706 | None |
| Genewiz | TFORF1517 | FOXD4L4 | NM_199244, ENST00000377413 | 1607 | 5157 | 8707 | None |
| Genewiz | TFORF1518 | FOXD4L5 | NM_001126334, ENST00000377420 | 1608 | 5158 | 8708 | None |
| Genewiz | TFORF1519 | FOXD4L6 | NM_001085476, ENST00000622588 | 1609 | 5159 | 8709 | None |
| Genewiz | TFORF1520 | NR2E1 | NM_001286102, ENST00000368983 | 1610 | 5160 | 8710 | None |
| Genewiz | TFORF1521 | NR2E3 | NM_016346, ENST00000621098 | 1611 | 5161 | 8711 | None |
| Genewiz | TFORF1522 | NR2E3 | NM_014249, ENST00000617575 | 1612 | 5162 | 8712 | None |
| Genewiz | TFORF1523 | NFRKB | NM_006165, XM_011542852, XM_011542851, | 1613 | 5163 | 8713 | None |
| | | | ENST00000524794 | | | | |
| Genewiz | TFORF1524 | NFRKB | NM_001143835, XM_017017796, ENST00000446488, | 1614 | 5164 | 8714 | None |
| | | | ENST00000524746 | | | | |
| Genewiz | TFORF1525 | GFI1 | XM_005270749, XM_011541245, XM_011541246, | 1615 | 5165 | 8715 | None |
| | | | NM_001127215, NM_001127216, NM_005263, | | | | |
| | | | ENST00000370332, ENST00000427103, ENST00000294702 | | | | |
| Genewiz | TFORF1526 | MXI1 | NM_005962, ENST00000239007 | 1616 | 5166 | 8716 | None |
| Genewiz | TFORF1527 | MXI1 | NM_001008541, ENST00000361248 | 1617 | 5167 | 8717 | None |
| Genewiz | TFORF1528 | MXI1 | NM_130439, ENST00000332674 | 1618 | 5168 | 8718 | None |
| Genewiz | TFORF1529 | ZNF605 | NM_001164715, ENST00000392321 | 1619 | 5169 | 8719 | None |
| Genewiz | TFORF1530 | ZNF605 | NM_183238, ENST00000360187 | 1620 | 5170 | 8720 | None |
| Genewiz | TFORF1531 | ZBTB40 | XM_011542499, NM_014870, NM_001083621, | 1621 | 5171 | 8721 | None |
| | | | ENST00000404138, ENST00000375647 | | | | |
| Genewiz | TFORF1532 | ZBTB40 | XM_017003003, ENST00000374651 | 1622 | 5172 | 8722 | None |
| Genewiz | TFORF1533 | ZBTB41 | NM_194314, ENST00000367405 | 1623 | 5173 | 8723 | None |
| Genewiz | TFORF1534 | ZBTB42 | XM_017020911, NM_001137601, ENST00000555360, | 1624 | 5174 | 8724 | None |
| | | | ENST00000342537 | | | | |
| Genewiz | TFORF1535 | ZBTB44 | NM_001301098, ENST00000397753, ENST00000357899 | 1625 | 5175 | 8725 | None |
| Genewiz | TFORF1536 | ZBTB44 | NM_001301099, XM_017017623, ENST00000530205 | 1626 | 5176 | 8726 | None |
| Genewiz | TFORF1537 | ZBTB45 | NM_001316978, NM_032792, NM_001316981, | 1627 | 5177 | 8727 | None |
| | | | NM_001316979, NM_001316982, NM_001316980, | | | | |
| | | | XM_006723445, ENST00000354590, ENST00000594051, | | | | |
| | | | ENST00000600990 | | | | |
| Genewiz | TFORF1538 | ZBTB46 | XM_005260197, XM_011528548, NM_025224, | 1628 | 5178 | 8728 | None |
| | | | XM_005260195, XM_006723700, XM_005260198, | | | | |
| | | | XM_005260196, ENST00000245663, ENST0395104, | | | | |
| | | | ENST00000302995 | | | | |
| Genewiz | TFORF1539 | ZBTB47 | NM_145166, ENST00000232974 | 1629 | 5179 | 8729 | None |
| Genewiz | TFORF1540 | ZBTB49 | NM_145291, XM_006713864, ENST00000337872 | 1630 | 5180 | 8730 | None |
| Genewiz | TFORF1541 | ZBTB49 | XM_017007835, ENST00000515012 | 1631 | 5181 | 8731 | None |
| Genewiz | TFORF1542 | ZNF286B | NM_001145045, ENST00000545289 | 1632 | 5182 | 8732 | None |
| Genewiz | TFORF1543 | ZNF160 | XM_017027446, NM_001322136, NM_001322130, | 1633 | 5183 | 8733 | None |
| | | | NM_001322131, NM_001322129, NM_001322134, | | | | |
| | | | NM_001322133, NM_001102603, NM_001322132, | | | | |
| | | | NM_198893, NM_001322128, NM_033288, | | | | |
| | | | NM_001322135, ENST00000599056, ENST00000418871, | | | | |
| | | | ENST00000429604 | | | | |
| Genewiz | TFORF1544 | ZNF160 | XM_017027448, NM_001322126, NM_001322125, | 1634 | 5184 | 8734 | None |
| | | | ENST00000355147 | | | | |
| Genewiz | TFORF1545 | ZNF160 | NM_001322139, NM_001322137, NM_001322138, | 1635 | 5185 | 8735 | None |
| | | | ENST00000601421 | | | | |
| Genewiz | TFORF1546 | BHMG1 | NM_001310124, ENST00000457052 | 1636 | 5186 | 8736 | None |
| Genewiz | TFORF1547 | SUPT5H | NM_001319991, NM_001130825, ENST00000402194, | 1637 | 5187 | 8737 | None |
| | | | ENST00000359191 | | | | |
| Genewiz | TFORF1548 | TAZ | NM_181311, ENST00000612460 | 1638 | 5188 | 8738 | None |
| Genewiz | TFORF1549 | TAZ | NM_181313, ENST00000613002 | 1639 | 5189 | 8739 | None |
| Genewiz | TFORF1550 | TAZ | NM_181312, ENST00000475699 | 1640 | 5190 | 8740 | None |
| Genewiz | TFORF1551 | TAZ | NM_000116, ENST00000601016 | 1641 | 5191 | 8741 | None |
| Genewiz | TFORF1552 | SP110 | NM_001185015, ENST00000540870 | 1642 | 5192 | 8742 | None |
| Genewiz | TFORF1553 | SP110 | NM_004510, ENST00000258382 | 1643 | 5193 | 8743 | None |
| Genewiz | TFORF1554 | SP110 | NM_004509, ENST00000358662 | 1644 | 5194 | 8744 | None |
| Genewiz | TFORF1555 | SP110 | NM_080424, ENST00000258381 | 1645 | 5195 | 8745 | None |
| Genewiz | TFORF1556 | YAF2 | XM_017018670, ENST00000552928 | 1646 | 5196 | 8746 | None |
| Genewiz | TFORF1557 | YAF2 | NM_005748, ENST00000534854 | 1647 | 5197 | 8747 | None |
| Genewiz | TFORF1558 | YAF2 | NM_001190980, ENST00000555248 | 1648 | 5198 | 8748 | None |
| Genewiz | TFORF1559 | YAF2 | NM_001190977, ENST00000380790 | 1649 | 5199 | 8749 | None |
| Genewiz | TFORF1560 | IL18 | NM_001243211, XM_011542805, ENST00000524595 | 1650 | 5200 | 8750 | None |
| Genewiz | TFORF1561 | ZNF688 | NM_145271, ENST00000223459 | 1651 | 5201 | 8751 | None |
| Genewiz | TFORF1562 | ZNF688 | NM_001024683, ENST00000563276 | 1652 | 5202 | 8752 | None |
| Genewiz | TFORF1563 | ZNF682 | XM_017027455, ENST00000597972 | 1653 | 5203 | 8753 | None |
| Genewiz | TFORF1564 | ZNF682 | NM_033196, ENST00000397165 | 1654 | 5204 | 8754 | None |
| Genewiz | TFORF1565 | ZNF682 | XM_017027456, ENST00000595736 | 1655 | 5205 | 8755 | None |
| Genewiz | TFORF1566 | ZNF682 | NM_001077349, ENST00000397162, ENST00000358523 | 1656 | 5206 | 8756 | None |
| Genewiz | TFORF1567 | ZNF683 | XM_005245832, XM_011541198, XM_005245830, | 1657 | 5207 | 8757 | None |
| | | | NM_001307925, ENST00000403843, ENST00000436292 | | | | |
| Genewiz | TFORF1568 | ZNF683 | NM_001114759, NM_173574, ENST00000349618 | 1658 | 5208 | 8758 | None |
| Genewiz | TFORF1569 | ZNF680 | NM_001130022, ENST00000447137 | 1659 | 5209 | 8759 | None |
| Genewiz | TFORF1570 | ZNF680 | NM_178558, ENST00000309683 | 1660 | 5210 | 8760 | None |
| Genewiz | TFORF1571 | ZNF681 | NM_138286, ENST00000402377 | 1661 | 5211 | 8761 | None |
| Genewiz | TFORF1572 | ZNF687 | NM_001304764, XM_011509812, XM_011509813, | 1662 | 5212 | 8762 | None |
| | | | NM_020832, NM_001304763, ENST00000336715, | | | | |
| | | | ENST00000324048 | | | | |
| Genewiz | TFORF1573 | ZNF684 | XM_011540672, XM_011540671, XM_017000290, | 1663 | 5213 | 8763 | None |
| | | | NM_152373, XM_011540673, ENST00000372699 | | | | |
| Genewiz | TFORF1574 | ZNF174 | NM_001032292, ENST00000575752, ENST00000344823 | 1664 | 5214 | 8764 | None |
| Genewiz | TFORF1575 | ZNF174 | NM_003450, ENST00000571936, ENST00000268655 | 1665 | 5215 | 8765 | None |
| Genewiz | TFORF1576 | ZNF177 | NM_001172651, ENST00000589262 | 1666 | 5216 | 8766 | None |
| Genewiz | TFORF1577 | AHR | NM_001621, ENST00000242057, ENST00000463496 | 1667 | 5217 | 8767 | None |
| Genewiz | TFORF1578 | ZNF778 | XM_011522940, NM_001201407, XM_005256288, | 1668 | 5218 | 8768 | None |
| | | | ENST00000433976 | | | | |
| Genewiz | TFORF1579 | ZNF778 | XM_017023015, ENST00000306502 | 1669 | 5219 | 8769 | None |
| Genewiz | TFORF1580 | ZNF778 | NM_182531, ENST00000620195 | 1670 | 5220 | 8770 | None |
| Genewiz | TFORF1581 | ATMIN | NM_015251, ENST00000299575 | 1671 | 5221 | 8771 | None |
| Genewiz | TFORF1582 | ZNF777 | NM_015694, XM_011516055, ENST00000247930 | 1672 | 5222 | 8772 | None |
| Genewiz | TFORF1583 | ZNF774 | NM_001004309, ENST00000354377 | 1673 | 5223 | 8773 | None |
| Genewiz | TFORF1584 | ZNF775 | NM_173680, ENST00000329630 | 1674 | 5224 | 8774 | None |
| Genewiz | TFORF1585 | ZNF772 | NM_001144068, ENST00000356584 | 1675 | 5225 | 8775 | None |
| Genewiz | TFORF1586 | ZNF772 | NM_001024596, ENST00000343280 | 1676 | 5226 | 8776 | None |
| Genewiz | TFORF1587 | ZNF772 | XM_005258943, XM_005258944, ENST00000427512 | 1677 | 5227 | 8777 | None |
| Genewiz | TFORF1588 | ZNF773 | NM_198542, ENST00000282292 | 1678 | 5228 | 8778 | None |
| Genewiz | TFORF1589 | ZNF773 | NM_001304334, ENST00000598770 | 1679 | 5229 | 8779 | None |
| Genewiz | TFORF1590 | ZNF773 | NM_001304337, ENST00000593916 | 1680 | 5230 | 8780 | None |
| Genewiz | TFORF1591 | CTCF | NM_001191022, ENST00000401394 | 1681 | 5231 | 8781 | None |
| Genewiz | TFORF1592 | ZNF771 | NM_001142305, NM_016643, ENST00000319296, | 1682 | 5232 | 8782 | None |
| | | | ENST00000434417 | | | | |
| Genewiz | TFORF1593 | ZNF197 | NM_001024855, XM_017005495, ENST00000383744, | 1683 | 5233 | 8783 | None |
| | | | ENST00000383745 | | | | |
| Genewiz | TFORF1594 | ZNF197 | XM_005264783, NM_006991, ENST00000344387, | 1684 | 5234 | 8784 | None |
| | | | ENST00000396058 | | | | |
| Genewiz | TFORF1595 | ZNF195 | NM_001130519, ENST00000005082 | 1685 | 5235 | 8785 | None |
| Genewiz | TFORF1596 | ZNF195 | NM_007152, ENST00000354599 | 1686 | 5236 | 8786 | None |
| Genewiz | TFORF1597 | ZNF195 | NM_001256825, NM_001242843, ENST00000343338, | 1687 | 5237 | 8787 | None |
| | | | ENST00000429541 | | | | |
| Genewiz | TFORF1598 | ZNF195 | NM_001242841, XM_017018263, ENST00000526601 | 1688 | 5238 | 8788 | None |
| Genewiz | TFORF1599 | ZNF195 | NM_001130520, ENST00000399602 | 1689 | 5239 | 8789 | None |
| Genewiz | TFORF1600 | ZNF195 | NM_001256823, ENST00000438262, ENST00000528218, | 1690 | 5240 | 8790 | None |
| | | | ENST00000618467 | | | | |
| Genewiz | TFORF1601 | ZNF195 | XM_011520350, XM_017018261, XM_011520351, | 1691 | 5241 | 8791 | None |
| | | | ENST00000620374 | | | | |
| Genewiz | TFORF1602 | ZNF256 | NM_005773, ENST00000282308 | 1692 | 5242 | 8792 | None |
| Genewiz | TFORF1603 | HINFP | NM_198971, NM_015517, XM_011542745, | 1693 | 5243 | 8793 | None |
| | | | ENST00000350777 | | | | |
| Genewiz | TFORF1604 | HINFP | NM_001243259, ENST00000527410 | 1694 | 5244 | 8794 | None |
| Genewiz | TFORF1605 | BAZ2A | NM_013449, ENST00000551812 | 1695 | 5245 | 8795 | None |
| Genewiz | TFORF1606 | BAZ2A | NM_001300905, ENST00000549884 | 1696 | 5246 | 8796 | None |
| Genewiz | TFORF1607 | BAZ2B | NM_001289975, ENST00000392782 | 1697 | 5247 | 8797 | None |
| Genewiz | TFORF1608 | BAZ2B | NM_013450, ENST00000392783 | 1698 | 5248 | 8798 | None |
| Genewiz | TFORF1609 | ZZZ3 | XM_005270729, NM_001308237, ENST00000370798 | 1699 | 5249 | 8799 | None |
| Genewiz | TFORF1610 | ZZZ3 | XM_005270725, NM_015534, XM_005270726, | 1700 | 5250 | 8800 | None |
| | | | ENST00000370801 | | | | |
| Genewiz | TFORF1611 | GSX2 | NM_133267, ENST00000611459, ENST00000326902 | 1701 | 5251 | 8801 | None |
| Genewiz | TFORF1612 | TADA2A | NM_001291918, NM_133439, ENST00000620367 | 1702 | 5252 | 8802 | None |
| Genewiz | TFORF1613 | TADA2A | NM_001166105, NM_001488, ENST00000615182, | 1703 | 5253 | 8803 | None |
| | | | ENST00000620628, ENST00000612272 | | | | |
| Genewiz | TFORF1614 | TADA2B | NM_152293, ENST00000310074 | 1704 | 5254 | 8804 | None |
| Genewiz | TFORF1615 | GSX1 | NM_145657, ENST00000302945 | 1705 | 5255 | 8805 | None |
| Genewiz | TFORF1616 | ZNF519 | XM_017025562, XM_017025563, XM_017025565, | 1706 | 5256 | 8806 | None |
| | | | XM_017025564, NM_145287, ENST00000590202 | | | | |
| Genewiz | TFORF1617 | NF1 | NM_001128147, ENST00000431387 | 1707 | 5257 | 8807 | None |
| Genewiz | TFORF1618 | NF1 | NM_001042492, ENST00000358273 | 1708 | 5258 | 8808 | None |
| Genewiz | TFORF1619 | NF1 | NM_000267, ENST00000356175 | 1709 | 5259 | 8809 | None |
| Genewiz | TFORF1620 | ZNF512 | NM_032434, ENST00000355467 | 1710 | 5260 | 8810 | None |
| Genewiz | TFORF1621 | ZNF512 | NM_001271286, ENST00000416005 | 1711 | 5261 | 8811 | None |
| Genewiz | TFORF1622 | ZNF513 | NM_001201459, XM_005264142, ENST00000407879 | 1712 | 5262 | 8812 | None |
| Genewiz | TFORF1623 | ZNF510 | NM_001314059, NM_014930, XM_017014483, | 1713 | 5263 | 8813 | None |
| | | | ENST00000223428, ENST00000375231 | | | | |
| Genewiz | TFORF1624 | ZNF516 | NM_014643, XM_011526271, XM_011526270, | 1714 | 5264 | 8814 | None |
| | | | XM_011526272, XM_011526269, XM_011526275, | | | | |
| | | | XM_017026097, XM_011526274, XM_011526273, | | | | |
| | | | ENST00000443185 | | | | |
| Genewiz | TFORF1625 | ZNF517 | NM_213605, XM_011517014, XM_011517015, | 1715 | 5265 | 8815 | None |
| | | | ENST00000359971, ENST00000533965, ENST00000531720 | | | | |
| Genewiz | TFORF1626 | ZNF514 | NM_032788, XM_006712806, ENST00000295208, | 1716 | 5266 | 8816 | None |
| | | | ENST00000411425 | | | | |
| Genewiz | TFORF1627 | FOXJ3 | NM_001198851, NM_014947, NM_001198850, | 1717 | 5267 | 8817 | None |
| | | | XM_011541026, ENST00000372572, ENST00000372573, | | | | |
| | | | ENST00000545068, ENST00000361346 | | | | |
| Genewiz | TFORF1628 | FOXJ3 | NM_001198852, ENST00000361776 | 1718 | 5268 | 8818 | None |
| Genewiz | TFORF1629 | ZNF224 | NM_013398, NM_001321645, XM_017027261, | 1719 | 5269 | 8819 | None |
| | | | ENST00000336976 | | | | |
| Genewiz | TFORF1630 | ZNF226 | NM_001146220, NM_015919, NM_001032374, | 1720 | 5270 | 8820 | None |
| | | | XM_017027265, ENST00000413984, ENST00000588742, | | | | |
| | | | ENST00000300823 | | | | |
| Genewiz | TFORF1631 | ZNF226 | NM_001319089, NM_001319088, NM_001319090, | 1721 | 5271 | 8821 | None |
| | | | XM_006723368, XM_006723367, NM_001032373, | | | | |
| | | | XM_006723369, XM_005259227, XM_017027262, | | | | |
| | | | NM_016444, NM_001032372, ENST00000337433, | | | | |
| | | | ENST0590089, ENST00000454662 | | | | |
| Genewiz | TFORF1632 | ZNF227 | XM_017027270, XM_017027271, NM_001289168, | 1722 | 5272 | 8822 | None |
| | | | NM_001289167, NM_001289169, XM_017027268, | | | | |
| | | | XM_017027269, ENST00000391961, ENST00000589005 | | | | |
| Genewiz | TFORF1633 | ZNF229 | NM_001278510, XM_011527292, ENST00000613197 | 1723 | 5273 | 8823 | None |
| Genewiz | TFORF1634 | ZNF229 | NM_014518, ENST00000614049 | 1724 | 5274 | 8824 | None |
| Genewiz | TFORF1635 | HOXC11 | NM_014212, ENST00000546378 | 1725 | 5275 | 8825 | None |
| Genewiz | TFORF1636 | HOXC13 | NM_017410, ENST00000243056 | 1726 | 5276 | 8826 | None |
| Genewiz | TFORF1637 | HOXC12 | NM_173860, ENST00000243103 | 1727 | 5277 | 8827 | None |
| Genewiz | TFORF1638 | BNIP3 | NM_004052, ENST00000368636 | 1728 | 5278 | 8828 | None |
| Genewiz | TFORF1639 | PSIP1 | NM_033222, NM_001128217, ENST00000380738, | 1729 | 5279 | 8829 | None |
| | | | ENST00000380733 | | | | |
| Genewiz | TFORF1640 | PSIP1 | NM_021144, ENST00000380716, ENST00000397519 | 1730 | 5280 | 8830 | None |
| Genewiz | TFORF1641 | PSIP1 | NM_001317898, ENST00000380715 | 1731 | 5281 | 8831 | None |
| Genewiz | TFORF1642 | ZNF426 | NM_001300883, XM_017027293, ENST00000593003 | 1732 | 5282 | 8832 | None |
| Genewiz | TFORF1643 | ZNF426 | NM_001318056, ENST00000589289 | 1733 | 5283 | 8833 | None |
| Genewiz | TFORF1644 | ZNF425 | NM_001001661, ENST00000378061 | 1734 | 5284 | 8834 | None |
| Genewiz | TFORF1645 | ZNF423 | XM_005255857, ENST00000535559, ENST00000567169 | 1735 | 5285 | 8835 | None |
| Genewiz | TFORF1646 | ZNF423 | NM_001271620, XM_017023078, XM_005255856, | 1736 | 5286 | 8836 | None |
| | | | XM_017023077, ENST00000563137, ENST00000562871, | | | | |
| | | | ENST00000562520 | | | | |
| Genewiz | TFORF1647 | ZNF358 | XM_005272460, NM_018083, XM_011527695, | 1737 | 5287 | 8837 | None |
| | | | ENST00000597229 | | | | |
| Genewiz | TFORF1648 | ZNF429 | NM_001001415, ENST00000358491 | 1738 | 5288 | 8838 | None |
| Genewiz | TFORF1649 | REL | NM_002908, ENST00000295025 | 1739 | 5289 | 8839 | None |
| Genewiz | TFORF1650 | ZSCAN5A | NM_001322075, NM_001322076, NM_001322073, | 1740 | 5290 | 8840 | None |
| | | | NM_001322074, XM_017027299, ENST00000592355 | | | | |
| Genewiz | TFORF1651 | ZSCAN5B | NM_001080456, XM_006723189, ENST00000586855, | 1741 | 5291 | 8841 | None |
| | | | ENST00000358992 | | | | |
| Genewiz | TFORF1652 | SPI1 | NM_001080547, ENST00000227163 | 1742 | 5292 | 8842 | None |
| Genewiz | TFORF1653 | SPI1 | NM_003120, ENST00000378538 | 1743 | 5293 | 8843 | None |
| Genewiz | TFORF1654 | TCFL5 | NM_006602, ENST00000335351 | 1744 | 5294 | 8844 | None |
| Genewiz | TFORF1655 | SPIB | NM_001243999, ENST00000270632 | 1745 | 5295 | 8845 | None |
| Genewiz | TFORF1656 | SPIB | NM_003121, ENST00000595883 | 1746 | 5296 | 8846 | None |
| Genewiz | TFORF1657 | SPIB | NM_001243998, ENST00000439922 | 1747 | 5297 | 8847 | None |
| Genewiz | TFORF1658 | LEF1 | NM_001166119, ENST00000510624 | 1748 | 5298 | 8848 | None |
| Genewiz | TFORF1659 | LEF1 | NM_001130714, ENST00000379951 | 1749 | 5299 | 8849 | None |
| Genewiz | TFORF1660 | LEF1 | NM_001130713, ENST00000438313 | 1750 | 5300 | 8850 | None |
| Genewiz | TFORF1661 | ARID5B | NM_001244638, ENST00000309334 | 1751 | 5301 | 8851 | None |
| Genewiz | TFORF1662 | ARIDSB | NM_032199, ENST00000279873 | 1752 | 5302 | 8852 | None |
| Genewiz | TFORF1663 | ARIDSA | NM_001319092, ENST00000454558 | 1753 | 5303 | 8853 | None |
| Genewiz | TFORF1664 | ARIDSA | NM_212481, ENST00000357485 | 1754 | 5304 | 8854 | None |
| Genewiz | TFORF1665 | TP73 | NM_005427, ENST00000378295 | 1755 | 5305 | 8855 | None |
| Genewiz | TFORF1666 | TP73 | NM_001126242, ENST00000378280 | 1756 | 5306 | 8856 | None |
| Genewiz | TFORF1667 | TP73 | NM_001204184, ENST00000354437 | 1757 | 5307 | 8857 | None |
| Genewiz | TFORF1668 | TP73 | NM_001204192, ENST00000378290 | 1758 | 5308 | 8858 | None |
| Genewiz | TFORF1669 | TP73 | NM_001204186, ENST00000604074 | 1759 | 5309 | 8859 | None |
| Genewiz | TFORF1670 | TP73 | NM_001204187, ENST00000357733, ENST00000603362 | 1760 | 5310 | 8860 | None |
| Genewiz | TFORF1671 | TP73 | NM_001126241, ENST00000378285 | 1761 | 5311 | 8861 | None |
| Genewiz | TFORF1672 | TP73 | NM_001126240, ENST00000378288 | 1762 | 5312 | 8862 | None |
| Genewiz | TFORF1673 | TP73 | NM_001204188, ENST00000346387, ENST00000604479 | 1763 | 5313 | 8863 | None |
| Genewiz | TFORF1674 | RUNX1T1 | NM_001198679, ENST00000436581 | 1764 | 5314 | 8864 | None |
| Genewiz | TFORF1675 | RUNX1T1 | NM_001198633, ENST00000615601 | 1765 | 5315 | 8865 | None |
| Genewiz | TFORF1676 | RUNX1T1 | NM_001198630, NM_001198629, NM_001198627, | 1766 | 5316 | 8866 | None |
| | | | NM_001198626, NM_001198631, NM_001198628, | | | | |
| | | | NM_175634, XM_017013931, XM_011517351, | | | | |
| | | | ENST00000613302, ENST00000614812, ENST00000617740, | | | | |
| | | | ENST00000265814, ENST00000523629 | | | | |
| Genewiz | TFORF1677 | RUNX1T1 | NM_001198634, ENST00000520724 | 1767 | 5317 | 8867 | None |
| Genewiz | TFORF1678 | RUNX1T1 | NM_004349, NM_001198632, NM_001198625, | 1768 | 5318 | 8868 | None |
| | | | XM_017013932, XM_017013933, XM_011517352, | | | | |
| | | | ENST00000613886, ENST00000396218, ENST00000518844 | | | | |
| Genewiz | TFORF1679 | RUNX1T1 | NM_175636, NM_175635, XM_011517353, | 1769 | 5319 | 8869 | None |
| | | | XM_017013935, XM_017013937, XM_017013934, | | | | |
| | | | XM_006716676, XM_017013936, ENST00000360348, | | | | |
| | | | ENST00000422361 | | | | |
| Genewiz | TFORF1680 | GTF2IRD2B | NM_001003795, ENST00000472837 | 1770 | 5320 | 8870 | None |
| Genewiz | TFORF1681 | SIM2 | NM_005069, ENST00000290399 | 1771 | 5321 | 8871 | None |
| Genewiz | TFORF1682 | SIM1 | XM_017011197, XM_005267100, NM_005068, | 1772 | 5322 | 8872 | None |
| | | | ENST00000369208, ENST00000262901 | | | | |
| Genewiz | TFORF1683 | TWIST1 | XM_011515496, NM_000474, ENST00000242261 | 1773 | 5323 | 8873 | None |
| Genewiz | TFORF1684 | HNF1B | NM_000458, ENST00000617811 | 1774 | 5324 | 8874 | None |
| Genewiz | TFORF1685 | HNF1B | XM_011525160, ENST00000614313 | 1775 | 5325 | 8875 | None |
| Genewiz | TFORF1686 | HNF1B | NM_001165923, ENST00000621123 | 1776 | 5326 | 8876 | None |
| Genewiz | TFORF1687 | HNF1B | NM_001304286, ENST00000613727 | 1777 | 5327 | 8877 | None |
| Genewiz | TFORF1688 | HNF1A | NM_001306179, ENST00000544413 | 1778 | 5328 | 8878 | None |
| Genewiz | TFORF1689 | HNF1A | XM_005253931, ENST00000541395 | 1779 | 5329 | 8879 | None |
| Genewiz | TFORF1690 | HNF1A | NM_000545, ENST00000257555 | 1780 | 5330 | 8880 | None |
| Genewiz | TFORF1691 | PGR | NM_001271162, ENST00000534013 | 1781 | 5331 | 8881 | None |
| Genewiz | TFORF1692 | ZNF814 | NM_001144989, ENST00000435989 | 1782 | 5332 | 8882 | None |
| Genewiz | TFORF1693 | E4F1 | NM_004424, ENST00000301727 | 1783 | 5333 | 8883 | None |
| Genewiz | TFORF1694 | E4F1 | NM_001288778, ENST00000565090 | 1784 | 5334 | 8884 | None |
| Genewiz | TFORF1695 | ZNF114 | NM_153608, XM_017026415, ENST00000595607, | 1785 | 5335 | 8885 | None |
| | | | ENST00000600687, ENST00000315849 | | | | |
| Genewiz | TFORF1696 | SOX21 | NM_007084, ENST00000376945 | 1786 | 5336 | 8886 | None |
| Genewiz | TFORF1697 | E2F6 | NM_001278277, NM_001278276, NM_001278278, | 1787 | 5337 | 8887 | None |
| | | | ENST00000542100, ENST00000546212 | | | | |
| Genewiz | TFORF1698 | E2F6 | NM_001278275, ENST00000307236 | 1788 | 5338 | 8888 | None |
| Genewiz | TFORF1699 | E2F5 | NM_001083588, ENST00000418930 | 1789 | 5339 | 8889 | None |
| Genewiz | TFORF1700 | E2F5 | NM_001951, ENST00000416274 | 1790 | 5340 | 8890 | None |
| Genewiz | TFORF1701 | E2F5 | NM_001083589, ENST00000517476 | 1791 | 5341 | 8891 | None |
| Genewiz | TFORF1702 | E2F4 | NM_001950, ENST00000379378 | 1792 | 5342 | 8892 | None |
| Genewiz | TFORF1703 | E2F3 | NM_001949, ENST00000346618 | 1793 | 5343 | 8893 | None |
| Genewiz | TFORF1704 | E2F3 | NM_001243076, ENST00000535432 | 1794 | 5344 | 8894 | None |
| Genewiz | TFORF1705 | E2F2 | NM_004091, ENST00000361729 | 1795 | 5345 | 8895 | None |
| Genewiz | TFORF1706 | E2F1 | NM_005225, ENST00000343380 | 1796 | 5346 | 8896 | None |
| Genewiz | TFORF1707 | BNC2 | NM_017637, ENST00000380672 | 1797 | 5347 | 8897 | None |
| Genewiz | TFORF1708 | PPP1R13L | NM_006663, NM_001142502, XM_017026177, | 1798 | 5348 | 8898 | None |
| | | | XM_017026178, ENST00000360957, ENST00000418234 | | | | |
| Genewiz | TFORF1709 | ZFP41 | NM_173832, NM_001271156, ENST00000520584, | 1799 | 5349 | 8899 | None |
| | | | ENST00000330701 | | | | |
| Genewiz | TFORF1710 | CBFB | NM_022845, ENST00000412916 | 1800 | 5350 | 8900 | None |
| Genewiz | TFORF1711 | FOXE1 | NM_004473, ENST00000375123 | 1801 | 5351 | 8901 | None |
| Genewiz | TFORF1712 | DPRX | XM_011527011, XM_011527012, NM_001012728, | 1802 | 5352 | 8902 | None |
| | | | ENST00000376650 | | | | |
| Genewiz | TFORF1713 | LDB2 | NM_001304434, ENST00000515064 | 1803 | 5353 | 8903 | None |
| Genewiz | TFORF1714 | LDB2 | NM_001130834, ENST00000441778 | 1804 | 5354 | 8904 | None |
| Genewiz | TFORF1715 | LDB2 | NM_001290, ENST00000304523 | 1805 | 5355 | 8905 | None |
| Genewiz | TFORF1716 | LDB2 | NM_001304435, ENST00000502640 | 1806 | 5356 | 8906 | None |
| Genewiz | TFORF1717 | LDB1 | NM_003893, ENST00000361198 | 1807 | 5357 | 8907 | None |
| Genewiz | TFORF1718 | LDB1 | NM_001113407, ENST00000425280 | 1808 | 5358 | 8908 | None |
| Genewiz | TFORF1719 | ZNF699 | NM_198535, ENST00000591998, ENST00000308650 | 1809 | 5359 | 8909 | None |
| Genewiz | TFORF1720 | DMRT2 | XM_011517690, XM_017014213, XM_011517687, | 1810 | 5360 | 8910 | None |
| | | | XM_017014215, XM_017014214, NM_181872, | | | | |
| | | | ENST00000382251, ENST00000358146 | | | | |
| Genewiz | TFORF1721 | DMRT2 | XM_017014216, NM_006557, NM_001130865, | 1811 | 5361 | 8911 | None |
| | | | ENST00000635183, ENST00000382255, ENST00000412350, | | | | |
| | | | ENST00000259622 | | | | |
| Genewiz | TFORF1722 | DMRT3 | NM_021240, ENST00000190165 | 1812 | 5362 | 8912 | None |
| Genewiz | TFORF1723 | ZNF691 | NM_001242739, XM_017001400, XM_017001401, | 1813 | 5363 | 8913 | None |
| | | | ENST00000372502 | | | | |
| Genewiz | TFORF1724 | ZNF691 | XM_017001402, XM_017001403, ENST00000372504, | 1814 | 5364 | 8914 | None |
| | | | ENST00000630961 | | | | |
| Genewiz | TFORF1725 | ZNF691 | NM_015911, ENST00000372508, ENST00000372507, | 1815 | 5365 | 8915 | None |
| | | | ENST00000372506 | | | | |
| Genewiz | TFORF1726 | ZNF692 | NM_001136036, ENST00000451251 | 1816 | 5366 | 8916 | None |
| Genewiz | TFORF1727 | ZNF692 | NM_001193328, ENST00000366471 | 1817 | 5367 | 8917 | None |
| Genewiz | TFORF1728 | ZNF695 | NM_001204221, ENST00000487338 | 1818 | 5368 | 8918 | None |
| Genewiz | TFORF1729 | ZNF695 | NM_020394, ENST00000339986 | 1819 | 5369 | 8919 | None |
| Genewiz | TFORF1730 | CRAMP1 | NM_020825, ENST00000397412, ENST00000293925 | 1820 | 5370 | 8920 | None |
| Genewiz | TFORF1731 | ZNF697 | NM_001080470, XM_011542416, XM_005271315, | 1821 | 5371 | 8921 | None |
| | | | ENST00000421812 | | | | |
| Genewiz | TFORF1732 | HDX | NM_001177478, XM_006724619, ENST00000506585 | 1822 | 5372 | 8922 | None |
| Genewiz | TFORF1733 | ZNF169 | NM_194320, ENST00000395395 | 1823 | 5373 | 8923 | None |
| Genewiz | TFORF1734 | RUVBL1 | NM_001319086, ENST00000464873 | 1824 | 5374 | 8924 | None |
| Genewiz | TFORF1735 | ADAR | NM_001111, ENST00000368474 | 1825 | 5375 | 8925 | None |
| Genewiz | TFORF1736 | ADAR | NM_001193495, NM_001025107, XM_006711111, | 1826 | 5376 | 8926 | None |
| | | | XM_006711113, XM_006711112, ENST00000368471 | | | | |
| Genewiz | TFORF1737 | ZBTB7B | XM_006711359, NM_001256455, XM_006711354, | 1827 | 5377 | 8927 | None |
| | | | XM_006711353, XM_006711349, XM_011509599, | | | | |
| | | | XM_006711357, XM_006711358, XM_006711356, | | | | |
| | | | ENST00000535420, ENST00000368426, ENST00000292176 | | | | |
| Genewiz | TFORF1738 | ZBTB7B | NM_001252406, XM_011509598, ENST00000417934 | 1828 | 5378 | 8928 | None |
| Genewiz | TFORF1739 | ZNF165 | NM_003447, XM_017011258, XM_017011260, | 1829 | 5379 | 8929 | None |
| | | | XM_017011259, ENST00000377325 | | | | |
| Genewiz | TFORF1740 | ZNF768 | XM_017023666, ENST00000562803 | 1830 | 5380 | 8930 | None |
| Genewiz | TFORF1741 | ZNF768 | NM_024671, ENST00000380412 | 1831 | 5381 | 8931 | None |
| Genewiz | TFORF1742 | ZNF765 | NM_001040185, ENST00000396408 | 1832 | 5382 | 8932 | None |
| Genewiz | TFORF1743 | ZNF764 | NM_001172679, ENST00000395091 | 1833 | 5383 | 8933 | None |
| Genewiz | TFORF1744 | ZNF764 | NM_033410, ENST00000252797 | 1834 | 5384 | 8934 | None |
| Genewiz | TFORF1745 | ZNF766 | NM_001010851, ENST00000439461 | 1835 | 5385 | 8935 | None |
| Genewiz | TFORF1746 | ZNF761 | NM_001289951, NM_001008401, NM_001289952, | 1836 | 5386 | 8936 | None |
| | | | ENST00000432094, ENST00000454407 | | | | |
| Genewiz | TFORF1747 | ZNF761 | NM_001289953, ENST00000613950 | 1837 | 5387 | 8937 | None |
| Genewiz | TFORF1748 | ZNF763 | NM_001012753, ENST00000343949 | 1838 | 5388 | 8938 | None |
| Genewiz | TFORF1749 | ZNF184 | NM_001318892, NM_001318891, NM_007149, | 1839 | 5389 | 8939 | None |
| | | | ENST00000211936, ENST00000377419 | | | | |
| Genewiz | TFORF1750 | ZNF181 | XM_017026739, XM_005258850, NM_001145665, | 1840 | 5390 | 8940 | None |
| | | | ENST00000459757 | | | | |
| Genewiz | TFORF1751 | ZNF180 | NM_001278508, ENST00000391956 | 1841 | 5391 | 8941 | None |
| Genewiz | TFORF1752 | ZNF180 | NM_013256, ENST00000221327 | 1842 | 5392 | 8942 | None |
| Genewiz | TFORF1753 | ZNF180 | NM_001278509, NM_001291633, ENST00000592529 | 1843 | 5393 | 8943 | None |
| Genewiz | TFORF1754 | ZNF182 | NM_001007088, ENST00000376943 | 1844 | 5394 | 8944 | None |
| Genewiz | TFORF1755 | ZNF182 | NM_006962, NM_001178099, ENST00000396965 | 1845 | 5395 | 8945 | None |
| Genewiz | TFORF1756 | ZNF189 | NM_001278240, ENST00000615466 | 1846 | 5396 | 8946 | None |
| Genewiz | TFORF1757 | ZNF189 | XM_006717281, XM_011518998, XM_006717280, | 1847 | 5397 | 8947 | None |
| | | | NM_197977, XM_017015121, ENST00000259395 | | | | |
| Genewiz | TFORF1758 | ZNF189 | NM_003452, ENST00000339664 | 1848 | 5398 | 8948 | None |
| Genewiz | TFORF1759 | ALYREF | NM_005782, ENST00000505490 | 1849 | 5399 | 8949 | None |
| Genewiz | TFORF1760 | ZNF25 | XM_005252385, XM_005252386, XM_005252384, | 1850 | 5400 | 8950 | None |
| | | | NM_145011, ENST00000302609 | | | | |
| Genewiz | TFORF1761 | ZNF24 | NM_006965, XM_005258341, ENST00000261332, | 1851 | 5401 | 8951 | None |
| | | | ENST00000399061 | | | | |
| Genewiz | TFORF1762 | ZNF24 | NM_001308123, ENST00000589881 | 1852 | 5402 | 8952 | None |
| Genewiz | TFORF1763 | ZNF26 | XM_011534829, XM_005266182, XM_017019923, | 1853 | 5403 | 8953 | None |
| | | | XM_005266183, XM_017019922, ENST00000534834 | | | | |
| Genewiz | TFORF1764 | ZNF20 | NM_021143, ENST00000334213 | 1854 | 5404 | 8954 | None |
| Genewiz | TFORF1765 | ZNF23 | NM_145911, NM_001304492, ENST00000393539, | 1855 | 5405 | 8955 | None |
| | | | ENST00000357254, ENST00000428724 | | | | |
| Genewiz | TFORF1766 | ZNF23 | NM_001304493, NM_001304494, ENST00000564528 | 1856 | 5406 | 8956 | None |
| Genewiz | TFORF1767 | ZNF22 | NM_006963, ENST00000298299 | 1857 | 5407 | 8957 | None |
| Genewiz | TFORF1768 | ZNF28 | NM_006969, XM_011527262, ENST00000457749 | 1858 | 5408 | 8958 | None |
| Genewiz | TFORF1769 | MGA | NM_001080541, ENST00000545763, ENST00000566586 | 1859 | 5409 | 8959 | None |
| Genewiz | TFORF1770 | MGA | NM_001164273, ENST00000219905, ENST00000570161 | 1860 | 5410 | 8960 | None |
| Genewiz | TFORF1771 | TBP | NM_003194, ENST00000392092, ENST00000230354 | 1861 | 5411 | 8961 | None |
| Genewiz | TFORF1772 | TBP | NM_001172085, ENST00000540980 | 1862 | 5412 | 8962 | None |
| Genewiz | TFORF1773 | ZNF501 | NM_145044, NM_001258280, ENST00000396048, | 1863 | 5413 | 8963 | None |
| | | | ENST00000620116 | | | | |
| Genewiz | TFORF1774 | ZNF500 | NM_021646, ENST00000219478 | 1864 | 5414 | 8964 | None |
| Genewiz | TFORF1775 | ZNF500 | NM_001303450, ENST00000545009 | 1865 | 5415 | 8965 | None |
| Genewiz | TFORF1776 | WHSC1 | NM_007331, ENST00000514045, ENST00000420906 | 1866 | 5416 | 8966 | None |
| Genewiz | TFORF1777 | WHSC1 | NM_001042424, XM_011513557, NM_133331, | 1867 | 5417 | 8967 | None |
| | | | NM_133330, NM_133335, XM_005248001, | | | | |
| | | | ENST00000508803, ENST00000382892, ENST082891, | | | | |
| | | | ENST00000382895 | | | | |
| Genewiz | TFORF1778 | WHSC1 | XM_005248005, XM_006713914, NM_133334, | 1868 | 5418 | 8968 | None |
| | | | ENST00000353275, ENST00000312087, ENST00000503128, | | | | |
| | | | ENST00000398261 | | | | |
| Genewiz | TFORF1779 | ZNF507 | NM_014910, NM_001136156, ENST00000311921, | 1869 | 5419 | 8969 | None |
| | | | ENST00000355898 | | | | |
| Genewiz | TFORF1780 | ZNF506 | NM_001099269, ENST00000591639, ENST00000443905, | 1870 | 5420 | 8970 | None |
| | | | ENST00000540806 | | | | |
| Genewiz | TFORF1781 | ZNF506 | NM_001145404, ENST00000450683 | 1871 | 5421 | 8971 | None |
| Genewiz | TFORF1782 | ZNF239 | XM_011540238, XM_005271832, XM_006718003, | 1872 | 5422 | 8972 | None |
| | | | NM_005674, NM_001099282, NM_001324349, | | | | |
| | | | NM_001324350, NM_001324351, NM_001324348, | | | | |
| | | | NM_001324347, NM_001099284, NM_001099283, | | | | |
| | | | ENST00000306006, ENST00000374446, ENST00000426961, | | | | |
| | | | ENST00000535642 | | | | |
| Genewiz | TFORF1783 | ZNF236 | NM_001306089, ENST00000320610 | 1873 | 5423 | 8973 | None |
| Genewiz | TFORF1784 | ZNF236 | XM_011526165, NM_007345, ENST00000253159 | 1874 | 5424 | 8974 | None |
| Genewiz | TFORF1785 | ZNF235 | NM_004234, ENST00000291182 | 1875 | 5425 | 8975 | None |
| Genewiz | TFORF1786 | ZNF234 | NM_001144824, NM_006630, XM_017026149, | 1876 | 5426 | 8976 | None |
| | | | XM_006722974, ENST00000592437, ENST00000426739 | | | | |
| Genewiz | TFORF1787 | TCERG1 | NM_006706, ENST00000296702 | 1877 | 5427 | 8977 | None |
| Genewiz | TFORF1788 | TCERG1 | NM_001040006, ENST00000394421 | 1878 | 5428 | 8978 | None |
| Genewiz | TFORF1789 | ZNF232 | NM_014519, XM_017025021, ENST00000250076 | 1879 | 5429 | 8979 | None |
| Genewiz | TFORF1790 | ZNF232 | NM_001320952, ENST00000575898 | 1880 | 5430 | 8980 | None |
| Genewiz | TFORF1791 | FOXE3 | NM_012186, ENST00000335071 | 1881 | 5431 | 8981 | None |
| Genewiz | TFORF1792 | CNBP | NM_001127194, ENST00000446936 | 1882 | 5432 | 8982 | None |
| Genewiz | TFORF1793 | CNBP | NM_001127193, ENST00000451728 | 1883 | 5433 | 8983 | None |
| Genewiz | TFORF1794 | CNBP | NM_001127192, ENST00000441626 | 1884 | 5434 | 8984 | None |
| Genewiz | TFORF1795 | CNBP | NM_003418, ENST00000422453 | 1885 | 5435 | 8985 | None |
| Genewiz | TFORF1796 | WDHD1 | NM_001008396, ENST00000420358 | 1886 | 5436 | 8986 | None |
| Genewiz | TFORF1797 | WDHD1 | NM_007086, ENST00000360586 | 1887 | 5437 | 8987 | None |
| Genewiz | TFORF1798 | USF2 | NM_003367, ENST00000222305 | 1888 | 5438 | 8988 | None |
| Genewiz | TFORF1799 | USF2 | NM_207291, ENST00000343550 | 1889 | 5439 | 8989 | None |
| Genewiz | TFORF1800 | USF2 | NM_001321150, ENST00000379134 | 1890 | 5440 | 8990 | None |
| Genewiz | TFORF1801 | TBX10 | NM_005995, ENST00000335385 | 1891 | 5441 | 8991 | None |
| Genewiz | TFORF1802 | USF3 | NM_001009899, XM_017005871, ENST00000316407, | 1892 | 5442 | 8992 | None |
| | | | ENST00000478658 | | | | |
| Genewiz | TFORF1803 | USF1 | NM_001276373, NM_007122, ENST00000368020, | 1893 | 5443 | 8993 | None |
| | | | ENST00000368021 | | | | |
| Genewiz | TFORF1804 | ZNF347 | XM_017027384, XM_005259335, NM_032584, | 1894 | 5444 | 8994 | None |
| | | | ENST00000334197 | | | | |
| Genewiz | TFORF1805 | ZNF347 | NM_001172674, NM_001172675, ENST00000452676, | 1895 | 5445 | 8995 | None |
| | | | ENST00000601469 | | | | |
| Genewiz | TFORF1806 | TBX15 | XM_005271162, ENST00000369429 | 1896 | 5446 | 8996 | None |
| Genewiz | TFORF1807 | ZNF432 | NM_001322285, NM_001322284, NM_014650, | 1897 | 5447 | 8997 | None |
| | | | ENST00000594154, ENST00000221315 | | | | |
| Genewiz | TFORF1808 | TBX18 | NM_001080508, ENST00000369663 | 1898 | 5448 | 8998 | None |
| Genewiz | TFORF1809 | TBX19 | NM_005149, ENST00000367821 | 1899 | 5449 | 8999 | None |
| Genewiz | TFORF1810 | ZNF439 | NM_152262, ENST00000304030 | 1900 | 5450 | 9000 | None |
| Genewiz | TFORF1811 | ZNF438 | XM_017015877, XM_017015875, XM_017015881, | 1901 | 5451 | 9001 | None |
| | | | XM_017015879, XM_017015878, XM_017015880, | | | | |
| | | | XM_017015882, XM_017015876, ENST00000375311 | | | | |
| Genewiz | TFORF1812 | ZNF438 | NM_001143770, NM_001143771, XM_017015873, | 1902 | 5452 | 9002 | None |
| | | | XM_011519376, XM_011519377, XM_006717399, | | | | |
| | | | XM_006717398, XM_017015874, ENST00000331737, | | | | |
| | | | ENST00000452305 | | | | |
| Genewiz | TFORF1813 | ZNF438 | NM_001143769, ENST00000538351 | 1903 | 5453 | 9003 | None |
| Genewiz | TFORF1814 | ZNF430 | NM_025189, ENST00000261560 | 1904 | 5454 | 9004 | None |
| Genewiz | TFORF1815 | ZNF433 | NM_001080411, ENST00000344980 | 1905 | 5455 | 9005 | None |
| Genewiz | TFORF1816 | SATB1 | NM_001195470, NM_001322871, XM_011533988, | 1906 | 5456 | 9006 | None |
| | | | XM_011533989, ENST00000417717 | | | | |
| Genewiz | TFORF1817 | UHRF1 | NM_013282, ENST00000622802 | 1907 | 5457 | 9007 | None |
| Genewiz | TFORF1818 | UHRF1 | NM_001290050, NM_001048201, NM_001290051, | 1908 | 5458 | 9008 | None |
| | | | NM_001290052, ENST00000612630, ENST00000624301, | | | | |
| | | | ENST00000615884, ENST00000616255 | | | | |
| Genewiz | TFORF1819 | ZNF138 | NM_006524, ENST00000440155 | 1909 | 5459 | 9009 | None |
| Genewiz | TFORF1820 | ZNF138 | NM_001271637, ENST00000440598 | 1910 | 5460 | 9010 | None |
| Genewiz | TFORF1821 | ZNF138 | NM_001271640, ENST00000359735 | 1911 | 5461 | 9011 | None |
| Genewiz | TFORF1822 | ZNF138 | NM_001160183, ENST00000494380 | 1912 | 5462 | 9012 | None |
| Genewiz | TFORF1823 | ZNF138 | NM_001271638, ENST00000437743 | 1913 | 5463 | 9013 | None |
| Genewiz | TFORF1824 | ZNF138 | NM_001271639, ENST00000307355 | 1914 | 5464 | 9014 | None |
| Genewiz | TFORF1825 | ARNT | NM_178427, ENST00000505755 | 1915 | 5465 | 9015 | None |
| Genewiz | TFORF1826 | ARNT | NM_001286036, ENST00000354396 | 1916 | 5466 | 9016 | None |
| Genewiz | TFORF1827 | ARNT | NM_001668, ENST00000358595 | 1917 | 5467 | 9017 | None |
| Genewiz | TFORF1828 | ARNT | NM_001286035, XM_017001289, ENST00000515192 | 1918 | 5468 | 9018 | None |
| Genewiz | TFORF1829 | DMRTA2 | XM_011541937, NM_032110, ENST00000418121, | 1919 | 5469 | 9019 | None |
| | | | ENST00000404795 | | | | |
| Genewiz | TFORF1830 | ZNF717 | NM_001324026, NM_001290210, XM_017005487, | 1920 | 5470 | 9020 | None |
| | | | ENST00000477374 | | | | |
| Genewiz | TFORF1831 | ZNF717 | NM_001290209, ENST00000478296 | 1921 | 5471 | 9021 | None |
| Genewiz | TFORF1832 | MTERF1 | NM_006980, XM_005250593, XM_006716126, | 1922 | 5472 | 9022 | None |
| | | | ENST00000351870 | | | | |
| Genewiz | TFORF1833 | MTERF1 | NM_001301135, NM_001301134, XM_017012620, | 1923 | 5473 | 9023 | None |
| | | | ENST00000419292, ENST00000406735 | | | | |
| Genewiz | TFORF1834 | EZH1 | NM_001321081, ENST00000415827 | 1924 | 5474 | 9024 | None |
| Genewiz | TFORF1835 | EZH1 | NM_001321082, ENST00000585893 | 1925 | 5475 | 9025 | None |
| Genewiz | TFORF1836 | EZH2 | NM_152998, ENST00000350995 | 1926 | 5476 | 9026 | None |
| Genewiz | TFORF1837 | EZH2 | NM_001203249, ENST00000478654, ENST00000476773 | 1927 | 5477 | 9027 | None |
| Genewiz | TFORF1838 | EZH2 | NM_001203247, ENST00000460911 | 1928 | 5478 | 9028 | None |
| Genewiz | TFORF1839 | EZH2 | NM_001203248, ENST00000483967 | 1929 | 5479 | 9029 | None |
| Genewiz | TFORF1840 | GTF2A1 | NM_201595, ENST00000434192 | 1930 | 5480 | 9030 | None |
| Genewiz | TFORF1841 | HBP1 | XM_017011967, NM_012257, XM_005250266, | 1931 | 5481 | 9031 | None |
| | | | XM_005250267, ENST00000468410, ENST00000222574, | | | | |
| | | | ENST00000485846 | | | | |
| Genewiz | TFORF1842 | YWHAZ | NM_001135702, NM_001135701, NM_001135700, | 1932 | 5482 | 9032 | None |
| | | | NM_145690, NM_001135699, NM_003406, | | | | |
| | | | XM_005251063, XM_017013811, XM_017013810, | | | | |
| | | | XM_005251061, ENST00000395957, ENST00000395958, | | | | |
| | | | ENST057309, ENST00000395956, ENST00000353245, | | | | |
| | | | ENST00000395953, ENST00000395951, ENST00000419477 | | | | |
| Genewiz | TFORF1843 | ALX1 | NM_006982, ENST00000316824 | 1933 | 5483 | 9033 | None |
| Genewiz | TFORF1844 | RB1 | NM_000321, ENST00000267163 | 1934 | 5484 | 9034 | None |
| Genewiz | TFORF1845 | ALX4 | NM_021926, ENST00000329255 | 1935 | 5485 | 9035 | None |
| Genewiz | TFORF1846 | ETS1 | NM_005238, ENST00000319397 | 1936 | 5486 | 9036 | None |
| Genewiz | TFORF1847 | ETS1 | NM_001143820, XM_017017314, ENST00000392668 | 1937 | 5487 | 9037 | None |
| Genewiz | TFORF1848 | ETS1 | XM_011542652, ENST00000526145 | 1938 | 5488 | 9038 | None |
| Genewiz | TFORF1849 | ETS1 | NM_001162422, ENST00000535549 | 1939 | 5489 | 9039 | None |
| Genewiz | TFORF1850 | YWHAE | NM_006761, ENST00000264335 | 1940 | 5490 | 9040 | None |
| Genewiz | TFORF1851 | ZBTB3 | NM_024784, ENST00000394807 | 1941 | 5491 | 9041 | None |
| Genewiz | TFORF1852 | SOX18 | NM_018419, ENST00000340356 | 1942 | 5492 | 9042 | None |
| Genewiz | TFORF1853 | ZBTB1 | NM_014950, XM_011536568, ENST00000358738 | 1943 | 5493 | 9043 | None |
| Genewiz | TFORF1854 | ZBTB5 | NM_014872, XM_005251634, ENST00000307750 | 1944 | 5494 | 9044 | None |
| Genewiz | TFORF1855 | ZBTB4 | NM_001128833, NM_020899, XM_006721563, | 1945 | 5495 | 9045 | None |
| | | | XM_006721564, XM_011523972, ENST00000380599, | | | | |
| | | | ENST00000311403 | | | | |
| Genewiz | TFORF1856 | NR4A2 | NM_006186, XM_017004219, ENST00000339562, | 1946 | 5496 | 9046 | None |
| | | | ENST00000409572 | | | | |
| Genewiz | TFORF1857 | NR4A2 | XM_005246622, ENST00000426264 | 1947 | 5497 | 9047 | None |
| Genewiz | TFORF1858 | NR4A3 | NM_006981, XM_017015162, ENST00000395097 | 1948 | 5498 | 9048 | None |
| Genewiz | TFORF1859 | NR4A3 | NM_173199, ENST00000338488 | 1949 | 5499 | 9049 | None |
| Genewiz | TFORF1860 | NR4A3 | NM_173200, ENST00000618101, ENST00000330847 | 1950 | 5500 | 9050 | None |
| Genewiz | TFORF1861 | DRGX | NM_001276451, ENST00000374139, ENST00000434016 | 1951 | 5501 | 9051 | None |
| Genewiz | TFORF1862 | OTP | NM_032109, ENST00000306422 | 1952 | 5502 | 9052 | None |
| Genewiz | TFORF1863 | POU2F2 | XM_005259010, ENST00000342301 | 1953 | 5503 | 9053 | None |
| Genewiz | TFORF1864 | POU2F2 | NM_001207025, ENST00000526816 | 1954 | 5504 | 9054 | None |
| Genewiz | TFORF1865 | POU2F2 | XM_011527042, ENST00000560398 | 1955 | 5505 | 9055 | None |
| Genewiz | TFORF1866 | POU2F2 | NM_002698, ENST00000389341 | 1956 | 5506 | 9056 | None |
| Genewiz | TFORF1867 | POU2F2 | NM_001207026, ENST00000529952 | 1957 | 5507 | 9057 | None |
| Genewiz | TFORF1868 | MMP3 | NM_002422, ENST00000299855 | 1958 | 5508 | 9058 | None |
| Genewiz | TFORF1869 | POU2F1 | NM_001198783, ENST00000367862 | 1959 | 5509 | 9059 | None |
| Genewiz | TFORF1870 | POU2F1 | NM_001198786, ENST00000429375 | 1960 | 5510 | 9060 | None |
| Genewiz | TFORF1871 | POU2F1 | NM_002697, ENST00000367866 | 1961 | 5511 | 9061 | None |
| Genewiz | TFORF1872 | BPTF | NM_182641, ENST00000306378 | 1962 | 5512 | 9062 | None |
| Genewiz | TFORF1873 | BBX | NM_020235, XM_017006882, XM_011513004, | 1963 | 5513 | 9063 | None |
| | | | ENST00000415149, ENST00000406780 | | | | |
| Genewiz | TFORF1874 | BBX | NM_001142568, XM_005247644, XM_011513001, | 1964 | 5514 | 9064 | None |
| | | | XM_011513000, XM_017006881, XM_005247642, | | | | |
| | | | XM_005247643, ENST00000325805 | | | | |
| Genewiz | TFORF1875 | CCNT1 | NM_001277842, ENST00000618666, ENST00000417344 | 1965 | 5515 | 9065 | None |
| Genewiz | TFORF1876 | CONT1 | NM_001240, ENST00000261900 | 1966 | 5516 | 9066 | None |
| Genewiz | TFORF1877 | CCNT2 | NM_001241, ENST00000295238 | 1967 | 5517 | 9067 | None |
| Genewiz | TFORF1878 | CCNT2 | NM_058241, ENST00000264157 | 1968 | 5518 | 9068 | None |
| Genewiz | TFORF1879 | CREBZF | NM_001039618, XM_017018089, XM_017018092, | 1969 | 5519 | 9069 | None |
| | | | XM_017018091, XM_017018088, XM_011545195, | | | | |
| | | | XM_017018090, ENST00000490820, ENST00000527447 | | | | |
| Genewiz | TFORF1880 | CREBZF | XM_017018087, XM_006718642, XM_017018086, | 1970 | 5520 | 9070 | None |
| | | | ENST00000525639 | | | | |
| Genewiz | TFORF1881 | SOX30 | NM_178424, ENST00000265007 | 1971 | 5521 | 9071 | None |
| Genewiz | TFORF1882 | SOX30 | NM_007017, ENST00000311371 | 1972 | 5522 | 9072 | None |
| Genewiz | TFORF1883 | SOX30 | XM_011534420, XM_005265803, NM_001308165, | 1973 | 5523 | 9073 | None |
| | | | ENST00000519442 | | | | |
| Genewiz | TFORF1884 | ZIC2 | NM_007129, ENST00000376335 | 1974 | 5524 | 9074 | None |
| Genewiz | TFORF1885 | BRCA1 | NM_007294, ENST00000357654 | 1975 | 5525 | 9075 | None |
| Genewiz | TFORF1886 | BRCA1 | NM_007300, ENST00000471181 | 1976 | 5526 | 9076 | None |
| Genewiz | TFORF1887 | BRCA1 | NM_007298, ENST00000491747 | 1977 | 5527 | 9077 | None |
| Genewiz | TFORF1888 | BRCA1 | NM_007297, ENST00000493795 | 1978 | 5528 | 9078 | None |
| Genewiz | TFORF1889 | WNK1 | NM_001184985, ENST00000537687 | 1979 | 5529 | 9079 | None |
| Genewiz | TFORF1890 | WNK1 | NM_014823, ENST00000535572 | 1980 | 5530 | 9080 | None |
| Genewiz | TFORF1891 | WNK1 | NM_018979, ENST00000315939 | 1981 | 5531 | 9081 | None |
| Genewiz | TFORF1892 | WNK1 | NM_213655, ENST00000340908 | 1982 | 5532 | 9082 | None |
| Genewiz | TFORF1893 | AHRR | NM_001242412, ENST00000505113 | 1983 | 5533 | 9083 | None |
| Genewiz | TFORF1894 | AHRR | NM_020731, ENST00000316418 | 1984 | 5534 | 9084 | None |
| Genewiz | TFORF1895 | NR2C2 | NM_001291694, XM_017007120, ENST00000425241, | 1985 | 5535 | 9085 | None |
| | | | ENST00000393102, ENST00000406272 | | | | |
| Genewiz | TFORF1896 | NR2C1 | NM_003297, ENST00000333003 | 1986 | 5536 | 9086 | None |
| Genewiz | TFORF1897 | NR2C1 | NM_001127362, ENST00000330677 | 1987 | 5537 | 9087 | None |
| Genewiz | TFORF1898 | PATZ1 | NM_014323, ENST00000266269 | 1988 | 5538 | 9088 | None |
| Genewiz | TFORF1899 | PATZ1 | NM_032050, ENST00000351933 | 1989 | 5539 | 9089 | None |
| Genewiz | TFORF1900 | PATZ1 | NM_032052, ENST00000405309 | 1990 | 5540 | 9090 | None |
| Genewiz | TFORF1901 | TCF7L1 | NM_031283, ENST00000282111 | 1991 | 5541 | 9091 | None |
| Genewiz | TFORF1902 | TCF7L2 | NM_001198528, ENST00000352065 | 1992 | 5542 | 9092 | None |
| Genewiz | TFORF1903 | TCF7L2 | XM_005270096, ENST00000538897 | 1993 | 5543 | 9093 | None |
| Genewiz | TFORF1904 | TCF7L2 | NM_001198530, ENST00000534894 | 1994 | 5544 | 9094 | None |
| Genewiz | TFORF1905 | TCF7L2 | XM_005270084, ENST00000355995 | 1995 | 5545 | 9095 | None |
| Genewiz | TFORF1906 | TCF7L2 | NM_001146274, ENST00000627217 | 1996 | 5546 | 9096 | None |
| Genewiz | TFORF1907 | TCF7L2 | NM_030756, ENST00000369397 | 1997 | 5547 | 9097 | None |
| Genewiz | TFORF1908 | TCF7L2 | NM_001146283, ENST00000355717 | 1998 | 5548 | 9098 | None |
| Genewiz | TFORF1909 | TCF7L2 | NM_001146285, ENST00000536810 | 1999 | 5549 | 9099 | None |
| Genewiz | TFORF1910 | TCF7L2 | XM_005270089, ENST00000629706 | 2000 | 5550 | 9100 | None |
| Genewiz | TFORF1911 | TCF7L2 | NM_001198527, ENST00000369395 | 2001 | 5551 | 9101 | None |
| Genewiz | TFORF1912 | TCF7L2 | NM_001198525, ENST00000545257 | 2002 | 5552 | 9102 | None |
| Genewiz | TFORF1913 | TCF7L2 | XM_005270085, ENST00000543371 | 2003 | 5553 | 9103 | None |
| Genewiz | TFORF1914 | RUNX2 | NM_001024630, ENST00000465038, ENST00000371438 | 2004 | 5554 | 9104 | None |
| Genewiz | TFORF1915 | RUNX2 | XM_017011396, ENST00000478660 | 2005 | 5555 | 9105 | None |
| Genewiz | TFORF1916 | RUNX2 | NM_001278478, ENST00000625924 | 2006 | 5556 | 9106 | None |
| Genewiz | TFORF1917 | RUNX2 | NM_001015051, ENST00000371432, ENST00000371436 | 2007 | 5557 | 9107 | None |
| Genewiz | TFORF1918 | RUNX3 | NM_004350, ENST00000308873 | 2008 | 5558 | 9108 | None |
| Genewiz | TFORF1919 | RUNX1 | NM_001122607, ENST00000358356 | 2009 | 5559 | 9109 | None |
| Genewiz | TFORF1920 | RUNX1 | NM_001001890, ENST00000344691 | 2010 | 5560 | 9110 | None |
| Genewiz | TFORF1921 | ZBTB22 | NM_005453, NM_001145338, ENST00000418724, | 2011 | 5561 | 9111 | None |
| | | | ENST00000431845 | | | | |
| Genewiz | TFORF1922 | ZBTB20 | NM_001164342, ENST00000474710 | 2012 | 5562 | 9112 | None |
| Genewiz | TFORF1923 | ZBTB20 | NM_001164347, NM_001164346, NM_001164344, | 2013 | 5563 | 9113 | None |
| | | | NM_001164345, NM_001164343, NM_015642, | | | | |
| | | | ENST00000357258, ENST00000462705, ENST00000393785, | | | | |
| | | | ENST00000481632, ENST00000471418, ENST00000464560 | | | | |
| Genewiz | TFORF1924 | ZBTB21 | NM_001320731, NM_001098402, NM_020727, | 2014 | 5564 | 9114 | None |
| | | | XM_011529590, XM_005261121, XM_017028361, | | | | |
| | | | XM_017028360, XM_011529588, ENST0310826, | | | | |
| | | | ENST00000398499, ENST00000398511 | | | | |
| Genewiz | TFORF1925 | ZBTB21 | NM_001098403, NM_001320729, ENST00000398505 | 2015 | 5565 | 9115 | None |
| Genewiz | TFORF1926 | ZBTB25 | NM_001304508, ENST00000555220 | 2016 | 5566 | 9116 | None |
| Genewiz | TFORF1927 | ZNF75D | NM_001185063, ENST00000370764 | 2017 | 5567 | 9117 | None |
| Genewiz | TFORF1928 | XRCC5 | NM_021141, ENST00000392133, ENST00000392132 | 2018 | 5568 | 9118 | None |
| Genewiz | TFORF1929 | XRCC4 | NM_003401, NM_022550, ENST00000282268, | 2019 | 5569 | 9119 | None |
| | | | ENST00000396027 | | | | |
| Genewiz | TFORF1930 | XRCC6 | NM_001288977, ENST00000402580 | 2020 | 5570 | 9120 | None |
| Genewiz | TFORF1931 | XRCC6 | NM_001288978, ENST00000428575, ENST00000405506 | 2021 | 5571 | 9121 | None |
| Genewiz | TFORF1932 | JUN | NM_002228, ENST00000371222 | 2022 | 5572 | 9122 | None |
| Genewiz | TFORF1933 | MEIS2 | NM_170677, ENST00000424352 | 2023 | 5573 | 9123 | None |
| Genewiz | TFORF1934 | MEIS2 | NM_170674, ENST00000559561 | 2024 | 5574 | 9124 | None |
| Genewiz | TFORF1935 | MEIS2 | NM_170675, ENST00000561208 | 2025 | 5575 | 9125 | None |
| Genewiz | TFORF1936 | MEIS2 | NM_172316, XM_017022205, ENST00000397624, | 2026 | 5576 | 9126 | None |
| | | | ENST00000397620 | | | | |
| Genewiz | TFORF1937 | MEIS2 | NM_172315, ENST00000559085 | 2027 | 5577 | 9127 | None |
| Genewiz | TFORF1938 | MEIS1 | NM_002398, ENST00000272369 | 2028 | 5578 | 9128 | None |
| Genewiz | TFORF1939 | MEIS1 | XM_005264325, ENST00000495021 | 2029 | 5579 | 9129 | None |
| Genewiz | TFORF1940 | JUP | NM_002230, NM_021991, XM_017024590, | 2030 | 5580 | 9130 | None |
| | | | XM_011524756, XM_011524758, XM_006721878, | | | | |
| | | | XM_011524753, XM_006721875, XM_011524755, | | | | |
| | | | XM_006721874, XM_011524757, XM_006721873, | | | | |
| | | | ENST00000393930, ENST00000310706, ENST00000393931 | | | | |
| Genewiz | TFORF1941 | MIER1 | NM_001077702, ENST00000371016 | 2031 | 5581 | 9131 | None |
| Genewiz | TFORF1942 | MIER1 | NM_020948, NM_001146110, XM_017001926, | 2032 | 5582 | 9132 | None |
| | | | XM_017001924, XM_005271076, XM_017001925, | | | | |
| | | | ENST00000357692 | | | | |
| Genewiz | TFORF1943 | MIER1 | NM_001077704, ENST00000401042 | 2033 | 5583 | 9133 | None |
| Genewiz | TFORF1944 | MIER1 | NM_001146111, ENST00000371018 | 2034 | 5584 | 9134 | None |
| Genewiz | TFORF1945 | MIER1 | NM_001077701, ENST00000355356 | 2035 | 5585 | 9135 | None |
| Genewiz | TFORF1946 | MIER1 | NM_001077703, ENST00000371014 | 2036 | 5586 | 9136 | None |
| Genewiz | TFORF1947 | MIER1 | NM_001278215, ENST00000371012 | 2037 | 5587 | 9137 | None |
| Genewiz | TFORF1948 | MIER1 | NM_001077700, ENST00000401041 | 2038 | 5588 | 9138 | None |
| Genewiz | TFORF1949 | MIER2 | NM_017550, ENST00000264819 | 2039 | 5589 | 9139 | None |
| Genewiz | TFORF1950 | MIER3 | NM_001297598, ENST00000381226 | 2040 | 5590 | 9140 | None |
| Genewiz | TFORF1951 | MIER3 | NM_152622, ENST00000381213 | 2041 | 5591 | 9141 | None |
| Genewiz | TFORF1952 | MIER3 | NM_001297599, ENST00000381199 | 2042 | 5592 | 9142 | None |
| Genewiz | TFORF1953 | PARP1 | NM_001618, ENST00000366794 | 2043 | 5593 | 9143 | None |
| Genewiz | TFORF1954 | ESR2 | NM_001271877, ENST00000267525 | 2044 | 5594 | 9144 | None |
| Genewiz | TFORF1955 | ESR2 | NM_001291712, NM_001040275, NM_001291723, | 2045 | 5595 | 9145 | None |
| | | | XM_017021084, ENST00000554572, ENST00000353772, | | | | |
| | | | ENST00000358599 | | | | |
| Genewiz | TFORF1956 | ESR2 | NM_001271876, ENST00000553796 | 2046 | 5596 | 9146 | None |
| Genewiz | TFORF1957 | ESR2 | NM_001214902, ENST00000555278 | 2047 | 5597 | 9147 | None |
| Genewiz | TFORF1958 | CUX1 | NM_181552, ENST00000292535 | 2048 | 5598 | 9148 | None |
| Genewiz | TFORF1959 | CUX1 | NM_001202543, ENST00000360264 | 2049 | 5599 | 9149 | None |
| Genewiz | TFORF1960 | CUX1 | NM_181500, ENST00000622516 | 2050 | 5600 | 9150 | None |
| Genewiz | TFORF1961 | CUX1 | NM_001202544, ENST00000547394 | 2051 | 5601 | 9151 | None |
| Genewiz | TFORF1962 | CUX1 | NM_001202546, ENST00000393824 | 2052 | 5602 | 9152 | None |
| Genewiz | TFORF1963 | CUX1 | NM_001913, ENST00000437600, ENST00000292538 | 2053 | 5603 | 9153 | None |
| Genewiz | TFORF1964 | CUX1 | NM_001202545, ENST00000425244 | 2054 | 5604 | 9154 | None |
| Genewiz | TFORF1965 | PPARD | NM_006238, NM_001171818, XM_006715123, | 2055 | 5605 | 9155 | None |
| | | | XM_017010973, XM_011514710, XM_006715120, | | | | |
| | | | XM_017010972, XM_011514709, XM_011514707, | | | | |
| | | | XM_017010971, XM_005249193, XM_017010974, | | | | |
| | | | ENST00000360694, ENST00000311565 | | | | |
| Genewiz | TFORF1966 | PPARD | NM_001171819, ENST00000448077 | 2056 | 5606 | 9156 | None |
| Genewiz | TFORF1967 | PPARD | NM_001171820, ENST00000418635 | 2057 | 5607 | 9157 | None |
| Genewiz | TFORF1968 | YY1 | NM_003403, ENST00000262238 | 2058 | 5608 | 9158 | None |
| Genewiz | TFORF1969 | ZNF233 | XM_017026759, XM_017026760, ENST00000592581 | 2059 | 5609 | 9159 | None |
| Genewiz | TFORF1970 | ZNF233 | NM_001207005, NM_181756, ENST00000391958 | 2060 | 5610 | 9160 | None |
| Genewiz | TFORF1971 | YY2 | NM_206923, ENST00000429584 | 2061 | 5611 | 9161 | None |
| Genewiz | TFORF1972 | TBX2 | NM_005994, ENST00000240328 | 2062 | 5612 | 9162 | None |
| Genewiz | TFORF1973 | TBX3 | NM_016569, ENST00000257566 | 2063 | 5613 | 9163 | None |
| Genewiz | TFORF1974 | TBX3 | NM_005996, ENST00000349155 | 2064 | 5614 | 9164 | None |
| Genewiz | TFORF1975 | ZNF34 | NM_030580, XM_017013874, XM_017013873, | 2065 | 5615 | 9165 | None |
| | | | XM_017013872, XM_011517314, ENST00000343459 | | | | |
| Genewiz | TFORF1976 | TBX1 | NM_080647, XM_017028926, XM_006724312, | 2066 | 5616 | 9166 | None |
| | | | ENST00000332710 | | | | |
| Genewiz | TFORF1977 | TBX1 | NM_005992, ENST00000359500 | 2067 | 5617 | 9167 | None |
| Genewiz | TFORF1978 | TBX1 | NM_080646, ENST00000329705 | 2068 | 5618 | 9168 | None |
| Genewiz | TFORF1979 | TBX6 | NM_004608, XM_017023614, XM_011545926, | 2069 | 5619 | 9169 | None |
| | | | XM_005255523, ENST00000395224, ENST00000279386 | | | | |
| Genewiz | TFORF1980 | TBX4 | NM_001321120, ENST00000393853 | 2070 | 5620 | 9170 | None |
| Genewiz | TFORF1981 | TBX4 | NM_018488, ENST00000240335 | 2071 | 5621 | 9171 | None |
| Genewiz | TFORF1982 | TBX5 | NM_080717, ENST00000349716 | 2072 | 5622 | 9172 | None |
| Genewiz | TFORF1983 | TBX5 | NM_181486, NM_000192, ENST00000310346, | 2073 | 5623 | 9173 | None |
| | | | ENST00000405440 | | | | |
| Genewiz | TFORF1984 | ZNF534 | NM_001291369, ENST00000432303 | 2074 | 5624 | 9174 | None |
| Genewiz | TFORF1985 | ZNF534 | NM_001291368, ENST00000301085, ENST00000617900 | 2075 | 5625 | 9175 | None |
| Genewiz | TFORF1986 | ZNF534 | NM_001143938, ENST00000433050 | 2076 | 5626 | 9176 | None |
| Genewiz | TFORF1987 | ZNF534 | NM_001143939, ENST00000332323 | 2077 | 5627 | 9177 | None |
| Genewiz | TFORF1988 | ZNF536 | XM_017027542, NM_014717, ENST00000355537 | 2078 | 5628 | 9178 | None |
| Genewiz | TFORF1989 | ZNF530 | NM_020880, ENST00000332854, ENST00000600619 | 2079 | 5629 | 9179 | None |
| Genewiz | TFORF1990 | ZNF532 | NM_001318726, NM_018181, XM_017025815, | 2080 | 5630 | 9180 | None |
| | | | XM_017025818, NM_001318727, XM_017025812, | | | | |
| | | | XM_017025816, NM_001318728, XM_017025814, | | | | |
| | | | XM_017025817, XM_017025813, ENST00000336078, | | | | |
| | | | ENST0591083, ENST00000589288, ENST00000591230, | | | | |
| | | | ENST00000591808 | | | | |
| Genewiz | TFORF1991 | ZHX2 | XM_005250837, NM_014943, XM_005250836, | 2081 | 5631 | 9181 | None |
| | | | XM_011516932, XM_011516931, ENST00000314393 | | | | |
| Genewiz | TFORF1992 | ZHX3 | NM_015035, XM_011528720, XM_017027737, | 2082 | 5632 | 9182 | None |
| | | | XM_017027738, ENST00000309060, ENST00000559234, | | | | |
| | | | ENST00000560361, ENST00000432768 | | | | |
| Genewiz | TFORF1993 | ZHX3 | XM_005260341, ENST00000544979 | 2083 | 5633 | 9183 | None |
| Genewiz | TFORF1994 | ZHX1 | NM_001017926, NM_007222, ENST00000297857, | 2084 | 5634 | 9184 | None |
| | | | ENST00000395571, ENST00000522655 | | | | |
| Genewiz | TFORF1995 | KDR | NM_002253, ENST00000263923 | 2085 | 5635 | 9185 | None |
| Genewiz | TFORF1996 | ZNF248 | XM_017016451, ENST00000485560 | 2086 | 5636 | 9186 | None |
| Genewiz | TFORF1997 | ZNF248 | XM_017016449, NM_001267607, ENST00000615949 | 2087 | 5637 | 9187 | None |
| Genewiz | TFORF1998 | ZNF248 | NM_001267606, NM_001267605, XM_011519599, | 2088 | 5638 | 9188 | None |
| | | | XM_017016450, XM_011519600, XM_011519598, | | | | |
| | | | ENST00000611278, ENST00000374648 | | | | |
| Genewiz | TFORF1999 | TAL1 | NM_001287347, NM_001290404, NM_001290405, | 2089 | 5639 | 9189 | None |
| | | | NM_003189, NM_001290403, XM_005271160, | | | | |
| | | | XM_017002187, XM_017002188, XM_017002190, | | | | |
| | | | XM_017002191, XM_017002189, ENST00000371884, | | | | |
| | | | ENST00000294339 | | | | |
| Genewiz | TFORF2000 | MYEF2 | NM_016132, ENST00000324324 | 2090 | 5640 | 9190 | None |
| Genewiz | TFORF2001 | MYEF2 | NM_001301210, ENST00000267836 | 2091 | 5641 | 9191 | None |
| Genewiz | TFORF2002 | FOXD2 | NM_004474, ENST00000334793 | 2092 | 5642 | 9192 | None |
| Genewiz | TFORF2003 | FOXD3 | NM_012183, ENST00000371116 | 2093 | 5643 | 9193 | None |
| Genewiz | TFORF2004 | FOXD1 | NM_004472, ENST00000615637 | 2094 | 5644 | 9194 | None |
| Genewiz | TFORF2005 | ZNF664 | NM_001204298, NM_152437, ENST00000539644, | 2095 | 5645 | 9195 | None |
| | | | ENST00000392404, ENST00000538932, ENST00000337815 | | | | |
| Genewiz | TFORF2006 | CAPN15 | NM_005632, ENST00000219611 | 2096 | 5646 | 9196 | None |
| Genewiz | TFORF2007 | PREB | XM_006711914, ENST00000406567 | 2097 | 5647 | 9197 | None |
| Genewiz | TFORF2008 | ZNF404 | NM_001033719, ENST00000587539 | 2098 | 5648 | 9198 | None |
| Genewiz | TFORF2009 | ZNF407 | NM_001146189, ENST00000577538 | 2099 | 5649 | 9199 | None |
| Genewiz | TFORF2010 | ZNF407 | XM_011526070, NM_001146190, ENST00000582337, | 2100 | 5650 | 9200 | None |
| | | | ENST00000309902 | | | | |
| Genewiz | TFORF2011 | ZNF407 | XM_005266726, XM_011526068, NM_017757, | 2101 | 5651 | 9201 | None |
| | | | ENST00000299687 | | | | |
| Genewiz | TFORF2012 | GATAD2A | XM_011528105, NM_001300946, ENST00000404158 | 2102 | 5652 | 9202 | None |
| Genewiz | TFORF2013 | GATAD2A | NM_017660, ENST00000360315, ENST00000358713 | 2103 | 5653 | 9203 | None |
| Genewiz | TFORF2014 | HMGXB3 | NM_014983, ENST00000502717 | 2104 | 5654 | 9204 | None |
| Genewiz | TFORF2015 | HMGXB4 | NM_001003681, ENST00000216106 | 2105 | 5655 | 9205 | None |
| Genewiz | TFORF2016 | PITX2 | NM_153427, NM_001204399, XM_011532027, | 2106 | 5656 | 9206 | None |
| | | | ENST00000355080, ENST00000616641 | | | | |
| Genewiz | TFORF2017 | PITX2 | NM_001204397, NM_153426, NM_001204398, | 2107 | 5657 | 9207 | None |
| | | | ENST00000394598, ENST00000354925, ENST00000613094, | | | | |
| | | | ENST00000614423 | | | | |
| Genewiz | TFORF2018 | PITX3 | NM_005029, ENST00000539804, ENST00000370002 | 2108 | 5658 | 9208 | None |
| Genewiz | TFORF2019 | MYBL2 | NM_001278610, ENST00000396863 | 2109 | 5659 | 9209 | None |
| Genewiz | TFORF2020 | MYBL2 | NM_002466, ENST00000217026 | 2110 | 5660 | 9210 | None |
| Genewiz | TFORF2021 | NPAS4 | XM_017017539, ENST00000525148 | 2111 | 5661 | 9211 | None |
| Genewiz | TFORF2022 | NPAS4 | NM_178864, ENST00000311034 | 2112 | 5662 | 9212 | None |
| Genewiz | TFORF2023 | NPAS3 | NM_173159, ENST00000357798 | 2113 | 5663 | 9213 | None |
| Genewiz | TFORF2024 | NPAS3 | NM_022123, ENST00000346562 | 2114 | 5664 | 9214 | None |
| Genewiz | TFORF2025 | NPAS3 | NM_001165893, ENST00000548645 | 2115 | 5665 | 9215 | None |
| Genewiz | TFORF2026 | NPAS3 | NM_001164749, ENST00000356141 | 2116 | 5666 | 9216 | None |
| Genewiz | TFORF2027 | NPAS2 | NM_002518, ENST00000335681 | 2117 | 5667 | 9217 | None |
| Genewiz | TFORF2028 | NPAS1 | NM_001321086, ENST00000439365 | 2118 | 5668 | 9218 | None |
| Genewiz | TFORF2029 | SMARCA5 | NM_003601, ENST00000283131 | 2119 | 5669 | 9219 | None |
| Genewiz | TFORF2030 | TGIF2LX | NM_138960, ENST00000283891, ENST00000561129 | 2120 | 5670 | 9220 | None |
| Genewiz | TFORF2031 | PRDM1 | NM_001198, ENST00000369096 | 2121 | 5671 | 9221 | None |
| Genewiz | TFORF2032 | PRDM1 | XM_017011187, XM_011536063, ENST00000369091 | 2122 | 5672 | 9222 | None |
| Genewiz | TFORF2033 | OSR1 | NM_145260, XM_006711942, ENST00000272223 | 2123 | 5673 | 9223 | None |
| Genewiz | TFORF2034 | OSR2 | NM_001286841, ENST00000457907 | 2124 | 5674 | 9224 | None |
| Genewiz | TFORF2035 | OSR2 | NM_001142462, XM_017013018, XM_011516827, | 2125 | 5675 | 9225 | None |
| | | | ENST00000297565, ENST00000522510 | | | | |
| Genewiz | TFORF2036 | TP53BP1 | XM_011521985, ENST00000382039 | 2126 | 5676 | 9226 | None |
| Genewiz | TFORF2037 | TP53BP1 | NM_001141979, ENST00000450115 | 2127 | 5677 | 9227 | None |
| Genewiz | TFORF2038 | TP53BP1 | NM_005657, ENST00000263801 | 2128 | 5678 | 9228 | None |
| Genewiz | TFORF2039 | TP53BP1 | NM_001141980, ENST00000382044 | 2129 | 5679 | 9229 | None |
| Genewiz | TFORF2040 | TP53BP2 | NM_001031685, ENST00000343537 | 2130 | 5680 | 9230 | None |
| Genewiz | TFORF2041 | ZNF850 | NM_001193552, ENST00000591344 | 2131 | 5681 | 9231 | None |
| Genewiz | TFORF2042 | ZNF850 | NM_001267779, ENST00000614887 | 2132 | 5682 | 9232 | None |
| Genewiz | TFORF2043 | ARID3A | XM_017026445, XM_005259514, NM_005224, | 2133 | 5683 | 9233 | None |
| | | | XM_005259513, ENST00000263620 | | | | |
| Genewiz | TFORF2044 | ARID3C | NM_001017363, ENST00000378909 | 2134 | 5684 | 9234 | None |
| Genewiz | TFORF2045 | ARID3B | NM_001307939, ENST00000622429 | 2135 | 5685 | 9235 | None |
| Genewiz | TFORF2046 | ARID3B | NM_006465, ENST00000346246 | 2136 | 5686 | 9236 | None |
| Genewiz | TFORF2047 | PRDM8 | XM_005263146, NM_020226, NM_001099403, | 2137 | 5687 | 9237 | None |
| | | | ENST00000504452, ENST00000339711, ENST00000415738 | | | | |
| Genewiz | TFORF2048 | PRDM9 | NM_020227, ENST00000296682 | 2138 | 5688 | 9238 | None |
| Genewiz | TFORF2049 | PURA | NM_005859, ENST00000331327 | 2139 | 5689 | 9239 | None |
| Genewiz | TFORF2050 | ATF6B | NM_004381, ENST00000375203 | 2140 | 5690 | 9240 | None |
| Genewiz | TFORF2051 | ATF6B | NM_001136153, ENST00000375201 | 2141 | 5691 | 9241 | None |
| Genewiz | TFORF2052 | BCL6 | NM_001134738, XM_011513062, ENST00000621333, | 2142 | 5692 | 9242 | None |
| | | | ENST00000450123 | | | | |
| Genewiz | TFORF2053 | BCL6 | NM_001130845, NM_001706, XM_005247694, | 2143 | 5693 | 9243 | None |
| | | | ENST00000406870, ENST00000232014 | | | | |
| Genewiz | TFORF2054 | BCL3 | NM_005178, ENST00000164227 | 2144 | 5694 | 9244 | None |
| Genewiz | TFORF2055 | CDX1 | NM_001804, ENST00000231656 | 2145 | 5695 | 9245 | None |
| Genewiz | TFORF2056 | NKX2-8 | NM_014360, ENST00000258829 | 2146 | 5696 | 9246 | None |
| Genewiz | TFORF2057 | ARGFX | NM_001012659, ENST00000334384 | 2147 | 5697 | 9247 | None |
| Genewiz | TFORF2058 | NKX2-2 | NM_002509, ENST00000377142 | 2148 | 5698 | 9248 | None |
| Genewiz | TFORF2059 | NKX2-3 | NM_145285, ENST00000344586 | 2149 | 5699 | 9249 | None |
| Genewiz | TFORF2060 | NKX2-1 | NM_001079668, ENST00000354822 | 2150 | 5700 | 9250 | None |
| Genewiz | TFORF2061 | NKX2-1 | NM_003317, ENST00000498187, ENST00000518149, | 2151 | 5701 | 9251 | None |
| | | | ENST00000522719 | | | | |
| Genewiz | TFORF2062 | NKX2-6 | NM_001136271, ENST00000325017 | 2152 | 5702 | 9252 | None |
| Genewiz | TFORF2063 | NKX2-4 | NM_033176, ENST00000351817 | 2153 | 5703 | 9253 | None |
| Genewiz | TFORF2064 | NKX2-5 | NM_001166176, ENST00000521848 | 2154 | 5704 | 9254 | None |
| Genewiz | TFORF2065 | NKX2-5 | NM_001166175, ENST00000424406 | 2155 | 5705 | 9255 | None |
| Genewiz | TFORF2066 | FLII | NM_001256265, ENST00000545457 | 2156 | 5706 | 9256 | None |
| Genewiz | TFORF2067 | FLII | NM_001256264, ENST00000579294 | 2157 | 5707 | 9257 | None |
| Genewiz | TFORF2068 | FLII | NM_002018, ENST00000327031 | 2158 | 5708 | 9258 | None |
| Genewiz | TFORF2069 | MYC | NM_002467, ENST00000621592, ENST00000613283 | 2159 | 5709 | 9259 | None |
| Genewiz | TFORF2070 | MYB | NM_001161660, ENST00000533624 | 2160 | 5710 | 9260 | None |
| Genewiz | TFORF2071 | MYB | NM_001161659, ENST00000534044 | 2161 | 5711 | 9261 | None |
| Genewiz | TFORF2072 | MYB | NM_005375, ENST00000367814 | 2162 | 5712 | 9262 | None |
| Genewiz | TFORF2073 | MYB | NM_001161656, ENST00000528774 | 2163 | 5713 | 9263 | None |
| Genewiz | TFORF2074 | MYB | NM_001161658, ENST00000534121 | 2164 | 5714 | 9264 | None |
| Genewiz | TFORF2075 | MYB | NM_001130173, ENST00000341911 | 2165 | 5715 | 9265 | None |
| Genewiz | TFORF2076 | MYB | NM_001161657, ENST00000525369 | 2166 | 5716 | 9266 | None |
| Genewiz | TFORF2077 | MYB | NM_001130172, ENST00000442647 | 2167 | 5717 | 9267 | None |
| Genewiz | TFORF2078 | FLI1 | NM_001271012, ENST00000344954 | 2168 | 5718 | 9268 | None |
| Genewiz | TFORF2079 | FLI1 | XM_017017405, XM_017017406, XM_011542701, | 2169 | 5719 | 9269 | None |
| | | | NM_001167681, ENST00000534087 | | | | |
| Genewiz | TFORF2080 | FLI1 | NM_001271010, ENST00000281428 | 2170 | 5720 | 9270 | None |
| Genewiz | TFORF2081 | FLI1 | NM_002017, ENST00000527786 | 2171 | 5721 | 9271 | None |
| Genewiz | TFORF2082 | IGHMBP2 | NM_002180, ENST00000255078 | 2172 | 5722 | 9272 | None |
| Genewiz | TFORF2083 | SNAPC4 | NM_003086, XM_006717242, XM_006717241, | 2173 | 5723 | 9273 | None |
| | | | XM_005266096, ENST00000298532 | | | | |
| Genewiz | TFORF2084 | ZNF441 | NM_152355, ENST00000357901 | 2174 | 5724 | 9274 | None |
| Genewiz | TFORF2085 | NFAT5 | NM_006599, ENST00000354436 | 2175 | 5725 | 9275 | None |
| Genewiz | TFORF2086 | NFAT5 | NM_138713, ENST00000627621 | 2176 | 5726 | 9276 | None |
| Genewiz | TFORF2087 | NFAT5 | NM_001113178, ENST00000567239 | 2177 | 5727 | 9277 | None |
| Genewiz | TFORF2088 | NFAT5 | XM_011522818, NM_173215, NM_173214, | 2178 | 5728 | 9278 | None |
| | | | NM_138714, XM_017022870, ENST00000349945, | | | | |
| | | | ENST00000393742, ENST00000566899 | | | | |
| Genewiz | TFORF2089 | ZNF442 | NM_030824, XM_017027318, ENST00000242804, | 2179 | 5729 | 9279 | None |
| | | | ENST00000545749 | | | | |
| Genewiz | TFORF2090 | FANCD2 | NM_033084, ENST00000287647 | 2180 | 5730 | 9280 | None |
| Genewiz | TFORF2091 | FANCD2 | NM_001018115, NM_001319984, ENST00000383807, | 2181 | 5731 | 9281 | None |
| | | | ENST00000419585 | | | | |
| Genewiz | TFORF2092 | ZBTB2 | NM_020861, XM_011536004, XM_005267076, | 2182 | 5732 | 9282 | None |
| | | | ENST00000325144 | | | | |
| Genewiz | TFORF2093 | ZFP14 | NM_020917, XM_017027038, ENST00000270001 | 2183 | 5733 | 9283 | None |
| Genewiz | TFORF2094 | DEK | NM_001134709, ENST00000244776 | 2184 | 5734 | 9284 | None |
| Genewiz | TFORF2095 | ST18 | NM_014682, XM_011517633, XM_017014063, | 2185 | 5735 | 9285 | None |
| | | | XM_017014058, XM_011517632, XM_017014061, | | | | |
| | | | XM_017014071, XM_017014067, XM_017014054, | | | | |
| | | | XM_017014065, XM_017014053, XM_017014068, | | | | |
| | | | XM_017014048, XM_017014047, XM_017014051, | | | | |
| | | | XM_017014069, XM_017014064, XM_011517636, | | | | |
| | | | XM_017014062, XM_017014070, XM_011517635, | | | | |
| | | | XM_011517634, XM_017014066, XM_011517631, | | | | |
| | | | XM_011517629, XM_006716487, XM_011517637, | | | | |
| | | | XM_017014056, XM_017014052, XM_017014049, | | | | |
| | | | XM_017014057, XM_017014060, XM_017014059, | | | | |
| | | | XM_017014050, XM_017014055, XM_011517638, | | | | |
| | | | ENST00000276480 | | | | |
| Genewiz | TFORF2096 | ZKSCAN1 | NM_001287055, ENST00000535170 | 2186 | 5736 | 9286 | None |
| Genewiz | TFORF2097 | ZKSCAN1 | NM_003439, XM_011516559, ENST00000324306 | 2187 | 5737 | 9287 | None |
| Genewiz | TFORF2098 | ZKSCAN1 | NM_001287054, ENST00000426572, ENST00000620510 | 2188 | 5738 | 9288 | None |
| Genewiz | TFORF2099 | SOHLH2 | NM_017826, ENST00000379881 | 2189 | 5739 | 9289 | None |
| Genewiz | TFORF2100 | SOHLH2 | NM_001282147, ENST00000317764 | 2190 | 5740 | 9290 | None |
| Genewiz | TFORF2101 | ZBTB38 | NM_001080412, XM_005247257, XM_011512611, | 2191 | 5741 | 9291 | None |
| | | | XM_017006061, XM_{——}017006055, XM_017006054, | | | | |
| | | | XM_017006053, XM_017006060, XM_017006056, | | | | |
| | | | XM_005247258, XM_017006052, XM_005247261, | | | | |
| | | | XM_017006059, XM_017006058, XM_017006057, | | | | |
| | | | ENST00000514251, ENST037056, ENST00000441582, | | | | |
| | | | ENST00000321464 | | | | |
| Genewiz | TFORF2102 | NEUROG2 | NM_024019, ENST00000313341 | 2192 | 5742 | 9292 | None |
| Genewiz | TFORF2103 | ZBTB32 | NM_014383, XM_017026589, XM_011526718, | 2193 | 5743 | 9293 | None |
| | | | ENST00000392197, ENST00000262630 | | | | |
| Genewiz | TFORF2104 | ZBTB34 | XM_011518699, ENST00000319119 | 2194 | 5744 | 9294 | None |
| Genewiz | TFORF2105 | ZBTB34 | NM_001099270, ENST00000373452 | 2195 | 5745 | 9295 | None |
| Genewiz | TFORF2106 | ZBTB37 | XM_017002558, XM_011510062, XM_017002557, | 2196 | 5746 | 9296 | None |
| | | | ENST00000367704 | | | | |
| Genewiz | TFORF2107 | ZBTB37 | XM_006711578, XM_005245546, NM_001122770, | 2197 | 5747 | 9297 | None |
| | | | XM_017002556, ENST00000427304, ENST00000367701 | | | | |
| Genewiz | TFORF2108 | L3MBTL4 | XM_011525761, NM_173464, ENST00000400105 | 2198 | 5748 | 9298 | None |
| Genewiz | TFORF2109 | L3MBTL4 | XM_005258166, ENST00000317931 | 2199 | 5749 | 9299 | None |
| Genewiz | TFORF2110 | NR4A1 | NM_001202233, ENST00000360284, ENST00000550082 | 2200 | 5750 | 9300 | None |
| Genewiz | TFORF2111 | NR4A1 | NM_001202234, ENST00000545748 | 2201 | 5751 | 9301 | None |
| Genewiz | TFORF2112 | SIRT6 | NM_001321063, ENST00000601488 | 2202 | 5752 | 9302 | None |
| Genewiz | TFORF2113 | SIRT6 | NM_001193285, ENST00000305232 | 2203 | 5753 | 9303 | None |
| Genewiz | TFORF2114 | SIRT6 | NM_001321064, ENST00000594279 | 2204 | 5754 | 9304 | None |
| Genewiz | TFORF2115 | NCOA6 | NM_001242539, XM_017027751, ENST00000612493 | 2205 | 5755 | 9305 | None |
| Genewiz | TFORF2116 | NCOA6 | NM_014071, NM_001318240, ENST00000374796, | 2206 | 5756 | 9306 | None |
| | | | ENST00000359003 | | | | |
| Genewiz | TFORF2117 | TBPL2 | NM_199047, ENST00000247219 | 2207 | 5757 | 9307 | None |
| Genewiz | TFORF2118 | TBPL1 | NM_001253676, XM_017011513, NM_004865, | 2208 | 5758 | 9308 | None |
| | | | ENST00000613034, ENST00000237264 | | | | |
| Genewiz | TFORF2119 | HOPX | NM_001145460, ENST00000554144 | 2209 | 5759 | 9309 | None |
| Genewiz | TFORF2120 | HOPX | NM_032495, ENST00000420433 | 2210 | 5760 | 9310 | None |
| Genewiz | TFORF2121 | HOPX | NM_001145459, NM_139211, NM_139212, | 2211 | 5761 | 9311 | None |
| | | | XM_017008734, XM_017008731, XM_017008730, | | | | |
| | | | XM_017008733, XM_017008729, XM_017008732, | | | | |
| | | | ENST00000337881, ENST00000503639, ENST00000553379, | | | | |
| | | | ENST0381255, ENST00000317745, ENST00000555760, | | | | |
| | | | ENST00000556614, ENST00000556376, ENST00000508121 | | | | |
| Genewiz | TFORF2122 | ZNF563 | XM_005259751, ENST00000595977 | 2212 | 5762 | 9312 | None |
| Genewiz | TFORF2123 | ZNF273 | XM_017011688, NM_021148, ENST00000476120 | 2213 | 5763 | 9313 | None |
| Genewiz | TFORF2124 | ZNF747 | NM_001305019, ENST00000568028 | 2214 | 5764 | 9314 | None |
| Genewiz | TFORF2125 | ZNF747 | NM_001305020, ENST00000395094 | 2215 | 5765 | 9315 | None |
| Genewiz | TFORF2126 | ZNF746 | NM_152557, ENST00000340622 | 2216 | 5766 | 9316 | None |
| Genewiz | TFORF2127 | ZNF746 | NM_001163474, ENST00000458143 | 2217 | 5767 | 9317 | None |
| Genewiz | TFORF2128 | ZNF740 | NM_001004304, ENST00000416904 | 2218 | 5768 | 9318 | None |
| Genewiz | TFORF2129 | ZNF749 | XM_017026802, NM_001023561, ENST00000334181 | 2219 | 5769 | 9319 | None |
| Genewiz | TFORF2130 | PFDN5 | NM_145897, ENST00000351500 | 2220 | 5770 | 9320 | None |
| Genewiz | TFORF2131 | ZGLP1 | NM_001103167, ENST00000403903 | 2221 | 5771 | 9321 | None |
| Genewiz | TFORF2132 | LCOR | XM_017016787, NM_001170765, XM_017016788, | 2222 | 5772 | 9322 | None |
| | | | XM_017016786, NM_032440, ENST00000371103, | | | | |
| | | | ENST00000356016, ENST00000371097 | | | | |
| Genewiz | TFORF2133 | MNAT1 | NM_001177963, ENST00000539616 | 2223 | 5773 | 9323 | None |
| Genewiz | TFORF2134 | ZNF521 | NM_015461, ENST00000361524, ENST00000538137 | 2224 | 5774 | 9324 | None |
| Genewiz | TFORF2135 | ZNF521 | NM_001308225, XM_017025698, XM_011525911, | 2225 | 5775 | 9325 | None |
| | | | ENST00000584787 | | | | |
| Genewiz | TFORF2136 | TRPS1 | NM_014112, XM_006716625, XM_011517264, | 2226 | 5776 | 9326 | None |
| | | | XM_011517266, ENST00000395715 | | | | |
| Genewiz | TFORF2137 | TRPS1 | XM_011517268, XM_005251049, ENST00000640765, | 2227 | 5777 | 9327 | None |
| | | | ENST00000220888 | | | | |
| Genewiz | TFORF2138 | TRPS1 | NM_001282902, ENST00000520276 | 2228 | 5778 | 9328 | None |
| Genewiz | TFORF2139 | ZNF527 | NM_032453, ENST00000436120 | 2229 | 5779 | 9329 | None |
| Genewiz | TFORF2140 | ZNF525 | ENST00000474037 | 2230 | 5780 | 9330 | None |
| Genewiz | TFORF2141 | ZNF525 | ENST00000491101 | 2231 | 5781 | 9331 | None |
| Genewiz | TFORF2142 | ZNF525 | ENST00000593918 | 2232 | 5782 | 9332 | None |
| Genewiz | TFORF2143 | ZNF525 | ENST00000467003 | 2233 | 5783 | 9333 | None |
| Genewiz | TFORF2144 | ZNF525 | ENST00000475179 | 2234 | 5784 | 9334 | None |
| Genewiz | TFORF2145 | ZNF529 | NM_020951, NM_001145649, XM_006723302, | 2235 | 5785 | 9335 | None |
| | | | XM_011527164, ENST00000591340 | | | | |
| Genewiz | TFORF2146 | ZNF529 | NM_001321351, XM_011527167, XM_017027044, | 2236 | 5786 | 9336 | None |
| | | | XM_017027041, XM_017027043, XM_017027042, | | | | |
| | | | ENST00000334116 | | | | |
| Genewiz | TFORF2147 | ZNF528 | NM_032423, ENST00000360465 | 2237 | 5787 | 9337 | None |
| Genewiz | TFORF2148 | ZNF251 | NM_138367, ENST00000292562 | 2238 | 5788 | 9338 | None |
| Genewiz | TFORF2149 | ZNF250 | NM_001109689, XM_011517209, XM_005272327, | 2239 | 5789 | 9339 | None |
| | | | XM_005272328, ENST00000417550 | | | | |
| Genewiz | TFORF2150 | ZNF250 | NM_021061, XM_006716613, XM_006716612, | 2240 | 5790 | 9340 | None |
| | | | ENST00000292579 | | | | |
| Genewiz | TFORF2151 | FOXG1 | NM_005249, ENST00000313071 | 2241 | 5791 | 9341 | None |
| Genewiz | TFORF2152 | BCL11A | NM_018014, ENST00000356842 | 2242 | 5792 | 9342 | None |
| Genewiz | TFORF2153 | BCL11A | XM_017004334, ENST00000358510 | 2243 | 5793 | 9343 | None |
| Genewiz | TFORF2154 | BCL11A | NM_138559, ENST00000359629 | 2244 | 5794 | 9344 | None |
| Genewiz | TFORF2155 | BCL11A | XM_011532909, NM_022893, ENST00000335712 | 2245 | 5795 | 9345 | None |
| Genewiz | TFORF2156 | RHOXF2 | NM_032498, ENST00000371388 | 2246 | 5796 | 9346 | None |
| Genewiz | TFORF2157 | RHOXF1 | NM_139282, ENST00000217999 | 2247 | 5797 | 9347 | None |
| Genewiz | TFORF2158 | BCL11B | NM_138576, ENST00000357195 | 2248 | 5798 | 9348 | None |
| Genewiz | TFORF2159 | BCL11B | NM_022898, ENST00000345514 | 2249 | 5799 | 9349 | None |
| Genewiz | TFORF2160 | TXK | NM_003328, ENST00000264316 | 2250 | 5800 | 9350 | None |
| Genewiz | TFORF2161 | GRHL1 | NM_198182, ENST00000324907 | 2251 | 5801 | 9351 | None |
| Genewiz | TFORF2162 | GRHL1 | XM_005246159, ENST00000405379 | 2252 | 5802 | 9352 | None |
| Genewiz | TFORF2163 | GRHL2 | XM_011517306, XM_011517305, ENST00000395927 | 2253 | 5803 | 9353 | None |
| Genewiz | TFORF2164 | GRHL2 | NM_024915, ENST00000251808 | 2254 | 5804 | 9354 | None |
| Genewiz | TFORF2165 | GRHL3 | NM_198173, ENST00000361548 | 2255 | 5805 | 9355 | None |
| Genewiz | TFORF2166 | GRHL3 | NM_198174, ENST00000350501 | 2256 | 5806 | 9356 | None |
| Genewiz | TFORF2167 | GRHL3 | NM_021180, ENST00000236255 | 2257 | 5807 | 9357 | None |
| Genewiz | TFORF2168 | GRHL3 | NM_001195010, XM_011541869, ENST00000356046 | 2258 | 5808 | 9358 | None |
| Genewiz | TFORF2169 | BLZF1 | NM_001320972, ENST00000367807 | 2259 | 5809 | 9359 | None |
| Genewiz | TFORF2170 | BHLHE22 | NM_152414, ENST00000321870 | 2260 | 5810 | 9360 | None |
| Genewiz | TFORF2171 | BHLHE23 | NM_080606, ENST00000612929 | 2261 | 5811 | 9361 | None |
| Genewiz | TFORF2172 | ZNF419 | NM_024691, ENST00000221735 | 2262 | 5812 | 9362 | None |
| Genewiz | TFORF2173 | ZNF419 | NM_001098496, ENST00000415379 | 2263 | 5813 | 9363 | None |
| Genewiz | TFORF2174 | ZNF419 | NM_001098491, ENST00000424930 | 2264 | 5814 | 9364 | None |
| Genewiz | TFORF2175 | ZNF419 | NM_001098494, ENST00000347466 | 2265 | 5815 | 9365 | None |
| Genewiz | TFORF2176 | ZNF419 | NM_001098492, ENST00000426954 | 2266 | 5816 | 9366 | None |
| Genewiz | TFORF2177 | ZNF419 | NM_001098493, ENST00000442920 | 2267 | 5817 | 9367 | None |
| Genewiz | TFORF2178 | ZNF418 | XM_017026305, NM_001317027, XM_017026304, | 2268 | 5818 | 9368 | None |
| | | | ENST00000425570 | | | | |
| Genewiz | TFORF2179 | ZNF418 | NM_001317030, ENST00000599852 | 2269 | 5819 | 9369 | None |
| Genewiz | TFORF2180 | ZNF417 | NM_152475, ENST00000312026 | 2270 | 5820 | 9370 | None |
| Genewiz | TFORF2181 | ZNF417 | NM_001297734, ENST00000595559 | 2271 | 5821 | 9371 | None |
| Genewiz | TFORF2182 | ZNF415 | XM_006723281, XM_017026972, XM_006723282, | 2272 | 5822 | 9372 | None |
| | | | ENST00000601493 | | | | |
| Genewiz | TFORF2183 | ZNF415 | NM_001164309, NM_018355, XM_017026964, | 2273 | 5823 | 9373 | None |
| | | | XM_011527103, ENST00000243643, ENST00000421033 | | | | |
| Genewiz | TFORF2184 | ZNF415 | NM_001136038, XM_017026966, XM_017026965, | 2274 | 5824 | 9374 | None |
| | | | XM_017026967, ENST00000500065 | | | | |
| Genewiz | TFORF2185 | ZNF414 | NM_032370, ENST00000255616 | 2275 | 5825 | 9375 | None |
| Genewiz | TFORF2186 | ZNF414 | NM_001146175, ENST00000393927 | 2276 | 5826 | 9376 | None |
| Genewiz | TFORF2187 | ZNF81 | XM_005272600, XM_017029486, NM_007137, | 2277 | 5827 | 9377 | None |
| | | | XM_011543900, XM_017029487, XM_011543899, | | | | |
| | | | ENST00000376954, ENST00000338637 | | | | |
| Genewiz | TFORF2188 | ZNF410 | NM_021188, ENST00000555044 | 2278 | 5828 | 9378 | None |
| Genewiz | TFORF2189 | ZNF410 | NM_001242924, ENST00000442160, ENST00000615736 | 2279 | 5829 | 9379 | None |
| Genewiz | TFORF2190 | ZNF410 | NM_001242927, ENST00000540593 | 2280 | 5830 | 9380 | None |
| Genewiz | TFORF2191 | RAX2 | NM_032753, ENST00000555978, ENST00000555633 | 2281 | 5831 | 9381 | None |
| Genewiz | TFORF2192 | RFXANK | NM_134440, NM_001278728, ENST00000392324 | 2282 | 5832 | 9382 | None |
| Genewiz | TFORF2193 | TFAM | NM_003201, ENST00000487519 | 2283 | 5833 | 9383 | None |
| Genewiz | TFORF2194 | TFAM | NM_001270782, ENST00000373895 | 2284 | 5834 | 9384 | None |
| Genewiz | TFORF2195 | MLXIPL | NM_032954, ENST00000354613 | 2285 | 5835 | 9385 | None |
| Genewiz | TFORF2196 | MLXIPL | NM_032953, ENST00000414749 | 2286 | 5836 | 9386 | None |
| Genewiz | TFORF2197 | MLXIPL | NM_032952, ENST00000429400 | 2287 | 5837 | 9387 | None |
| Genewiz | TFORF2198 | MLXIPL | NM_032951, ENST00000313375 | 2288 | 5838 | 9388 | None |
| Genewiz | TFORF2199 | RXRA | NM_002957, ENST00000481739 | 2289 | 5839 | 9389 | None |
| Genewiz | TFORF2200 | POU4F1 | NM_006237, ENST00000377208 | 2290 | 5840 | 9390 | None |
| Genewiz | TFORF2201 | POU4F2 | NM_004575, ENST00000281321 | 2291 | 5841 | 9391 | None |
| Genewiz | TFORF2202 | POU4F3 | NM_002700, ENST00000230732 | 2292 | 5842 | 9392 | None |
| Genewiz | TFORF2203 | MYNN | XM_011512987, XM_017006864, NM_018657, | 2293 | 5843 | 9393 | None |
| | | | NM_001185118, ENST00000349841, ENST00000356716 | | | | |
| Genewiz | TFORF2204 | MYNN | NM_001185119, ENST00000544106 | 2294 | 5844 | 9394 | None |
| Genewiz | TFORF2205 | POU2F3 | NM_001244682, ENST00000260264 | 2295 | 5845 | 9395 | None |
| Genewiz | TFORF2206 | POU2F3 | NM_014352, ENST00000543440 | 2296 | 5846 | 9396 | None |
| Genewiz | TFORF2207 | CREB3L2 | NM_194071, ENST00000330387 | 2297 | 5847 | 9397 | None |
| Genewiz | TFORF2208 | CREB3L2 | NM_001253775, ENST00000452463 | 2298 | 5848 | 9398 | None |
| Genewiz | TFORF2209 | CREB3L3 | NM_032607, ENST00000078445 | 2299 | 5849 | 9399 | None |
| Genewiz | TFORF2210 | CREB3L3 | NM_001271997, ENST00000602147 | 2300 | 5850 | 9400 | None |
| Genewiz | TFORF2211 | CREB3L3 | NM_001271995, ENST00000595923 | 2301 | 5851 | 9401 | None |
| Genewiz | TFORF2212 | CREB3L3 | NM_001271996, ENST00000602257 | 2302 | 5852 | 9402 | None |
| Genewiz | TFORF2213 | CREB3L1 | NM_052854, ENST00000621158 | 2303 | 5853 | 9403 | None |
| Genewiz | TFORF2214 | CREB3L4 | XM_017000372, NM_001255980, XM_006711172, | 2304 | 5854 | 9404 | None |
| | | | NM_001255981, ENST00000368600 | | | | |
| Genewiz | TFORF2215 | CREB3L4 | NM_001255978, NM_130898, NM_001255979, | 2305 | 5855 | 9405 | None |
| | | | ENST00000368607, ENST00000271889, ENST00000368603 | | | | |
| Genewiz | TFORF2216 | NOTO | NM_001134462, ENST00000398468 | 2306 | 5856 | 9406 | None |
| Genewiz | TFORF2217 | YAP1 | NM_001282098, ENST00000629586 | 2307 | 5857 | 9407 | None |
| Genewiz | TFORF2218 | YAP1 | NM_001282099, ENST00000345877 | 2308 | 5858 | 9408 | None |
| Genewiz | TFORF2219 | YAP1 | NM_001130145, ENST00000282441 | 2309 | 5859 | 9409 | None |
| Genewiz | TFORF2220 | YAP1 | NM_006106, ENST00000526343 | 2310 | 5860 | 9410 | None |
| Genewiz | TFORF2221 | YAP1 | NM_001282101, ENST00000615667 | 2311 | 5861 | 9411 | None |
| Genewiz | TFORF2222 | YAP1 | NM_001195044, ENST00000531439 | 2312 | 5862 | 9412 | None |
| Genewiz | TFORF2223 | YAP1 | NM_001195045, ENST00000524575 | 2313 | 5863 | 9413 | None |
| Genewiz | TFORF2224 | YAP1 | NM_001282100, ENST00000537274 | 2314 | 5864 | 9414 | None |
| Genewiz | TFORF2225 | ZNF343 | NM_001282498, ENST00000617391 | 2315 | 5865 | 9415 | None |
| Genewiz | TFORF2226 | ZNF343 | NM_001321801, NM_001282497, ENST00000612935 | 2316 | 5866 | 9416 | None |
| Genewiz | TFORF2227 | ZNF343 | NM_001282499, ENST00000381253, ENST00000358413 | 2317 | 5867 | 9417 | None |
| Genewiz | TFORF2228 | ZNF343 | NM_001321800, NM_024325, NM_001282496, | 2318 | 5868 | 9418 | None |
| | | | NM_001282495, XM_017028062, ENST00000278772 | | | | |
| Genewiz | TFORF2229 | ZNF730 | NM_001277403, ENST00000597761 | 2319 | 5869 | 9419 | None |
| Genewiz | TFORF2230 | EHF | NM_001206615, XM_005252862, ENST00000450654 | 2320 | 5870 | 9420 | None |
| Genewiz | TFORF2231 | EHF | NM_012153, XM_011519984, XM_005252860, | 2321 | 5871 | 9421 | None |
| | | | XM_011519985, ENST00000257831, ENST00000530286, | | | | |
| | | | ENST00000533754 | | | | |
| Genewiz | TFORF2232 | EHF | NM_001206616, ENST00000531794 | 2322 | 5872 | 9422 | None |
| Genewiz | TFORF2233 | SMARCD1 | NM_139071, ENST00000381513 | 2323 | 5873 | 9423 | None |
| Genewiz | TFORF2234 | GATA6 | NM_005257, ENST00000269216, ENST00000581694 | 2324 | 5874 | 9424 | None |
| Genewiz | TFORF2235 | ZNF880 | XM_017026812, NM_001145434, ENST00000422689 | 2325 | 5875 | 9425 | None |
| Genewiz | TFORF2236 | GATA4 | NM_001308094, ENST00000528712 | 2326 | 5876 | 9426 | None |
| Genewiz | TFORF2237 | GATA4 | XM_005272386, XM_011543817, XM_006716248, | 2327 | 5877 | 9427 | None |
| | | | XM_011543818, XM_017013312, XM_005272385, | | | | |
| | | | NM_001308093, ENST00000532059 | | | | |
| Genewiz | TFORF2238 | GATA4 | NM_002052, ENST00000335135 | 2328 | 5878 | 9428 | None |
| Genewiz | TFORF2239 | GATA5 | NM_080473, XM_006723699, ENST00000252997 | 2329 | 5879 | 9429 | None |
| Genewiz | TFORF2240 | GATA3 | NM_002051, ENST00000346208 | 2330 | 5880 | 9430 | None |
| Genewiz | TFORF2241 | GATA3 | XM_005252443, XM_005252442, NM_001002295, | 2331 | 5881 | 9431 | None |
| | | | ENST00000379328 | | | | |
| Genewiz | TFORF2242 | GATA1 | NM_002049, ENST00000376670 | 2332 | 5882 | 9432 | None |
| Genewiz | TFORF2243 | LITAR | NM_001136473, ENST00000413364, ENST00000620789 | 2333 | 5883 | 9433 | None |
| Genewiz | TFORF2244 | TCF15 | NM_004609, ENST00000246080 | 2334 | 5884 | 9434 | None |
| Genewiz | TFORF2245 | TCF12 | NM_001306219, ENST00000543579 | 2335 | 5885 | 9435 | None |
| Genewiz | TFORF2246 | TCF12 | NM_207040, ENST00000343827 | 2336 | 5886 | 9436 | None |
| Genewiz | TFORF2247 | TCF12 | NM_207038, NM_003205, NM_001322157, | 2337 | 5887 | 9437 | None |
| | | | NM_001322165, ENST0267811, ENST00000557843 | | | | |
| Genewiz | TFORF2248 | TCF12 | NM_001306220, ENST00000537840 | 2338 | 5888 | 9438 | None |
| Genewiz | TFORF2249 | TCF12 | NM_001322151, NM_207036, NM_207037, | 2339 | 5889 | 9439 | None |
| | | | NM_001322159, NM_001322162, ENST00000438423, | | | | |
| | | | ENST00000333725 | | | | |
| Genewiz | TFORF2250 | TCF19 | NM_001318908, NM_001077511, NM_007109, | 2340 | 5890 | 9440 | None |
| | | | ENST00000376257, ENST00000376255 | | | | |
| Genewiz | TFORF2251 | ZNF630 | NM_001037735, NM_001282201, ENST00000442455, | 2341 | 5891 | 9441 | None |
| | | | ENST00000409324 | | | | |
| Genewiz | TFORF2252 | ZNF630 | NM_001282202, ENST00000276054 | 2342 | 5892 | 9442 | None |
| Genewiz | TFORF2253 | HMGN3 | NM_004242, ENST00000344726 | 2343 | 5893 | 9443 | None |
| Genewiz | TFORF2254 | HMGN3 | NM_001201363, ENST00000620514 | 2344 | 5894 | 9444 | None |
| Genewiz | TFORF2255 | ARNTL | NM_001297722, NM_001297719, XM_017017742, | 2345 | 5895 | 9445 | None |
| | | | ENST00000403290 | | | | |
| Genewiz | TFORF2256 | ARNTL | NM_001297724, XM_017017746, XM_011520112, | 2346 | 5896 | 9446 | None |
| | | | XM_017017747, ENST00000401424 | | | | |
| Genewiz | TFORF2257 | ARNTL | NM_001030273, XM_011520113, XM_017017748, | 2347 | 5897 | 9447 | None |
| | | | ENST00000403510 | | | | |
| Genewiz | TFORF2258 | SOX11 | NM_003108, ENST00000322002 | 2348 | 5898 | 9448 | None |
| Genewiz | TFORF2259 | SOX13 | NM_005686, ENST00000367204, ENST00000618875 | 2349 | 5899 | 9449 | None |
| Genewiz | TFORF2260 | SOX17 | NM_022454, ENST00000297316 | 2350 | 5900 | 9450 | None |
| Genewiz | TFORF2261 | DMAP1 | NM_001034024, NM_001034023, NM_019100, | 2351 | 5901 | 9451 | None |
| | | | XM_017001808, ENST00000361745, ENST00000315913, | | | | |
| | | | ENST00000372289 | | | | |
| Genewiz | TFORF2262 | ZFP28 | NM_001308440, ENST00000591844 | 2352 | 5902 | 9452 | None |
| Genewiz | TFORF2263 | ZFP28 | NM_020828, ENST00000301318 | 2353 | 5903 | 9453 | None |
| Genewiz | TFORF2264 | ZNF286A | NM_001130842, NM_020652, ENST00000421016, | 2354 | 5904 | 9454 | None |
| | | | ENST00000583566, ENST00000464847 | | | | |
| Genewiz | TFORF2265 | ZNF286A | NM_001288643, ENST00000593105 | 2355 | 5905 | 9455 | None |
| Genewiz | TFORF2266 | ANHX | NM_001191054, ENST00000419717, ENST00000545940 | 2356 | 5906 | 9456 | None |
| Genewiz | TFORF2267 | ARNT2 | NM_014862, ENST00000303329 | 2357 | 5907 | 9457 | None |
| Genewiz | TFORF2268 | ZNF701 | NM_001172655, XM_011527092, ENST00000301093, | 2358 | 5908 | 9458 | None |
| | | | ENST00000540331 | | | | |
| Genewiz | TFORF2269 | ZNF701 | NM_018260, ENST00000391785 | 2359 | 5909 | 9459 | None |
| Genewiz | TFORF2270 | PSAP | NM_002778, ENST00000394936 | 2360 | 5910 | 9460 | None |
| Genewiz | TFORF2271 | PRDM5 | NM_001300824, ENST00000515109 | 2361 | 5911 | 9461 | None |
| Genewiz | TFORF2272 | PRDM5 | NM_018699, ENST00000264808 | 2362 | 5912 | 9462 | None |
| Genewiz | TFORF2273 | PRDM6 | NM_001136239, ENST00000407847 | 2363 | 5913 | 9463 | None |
| Genewiz | TFORF2274 | SLC22A1 | XM_006715552, ENST00000457470 | 2364 | 5914 | 9464 | None |
| Genewiz | TFORF2275 | SLC22A1 | NM_003057, ENST00000366963 | 2365 | 5915 | 9465 | None |
| Genewiz | TFORF2276 | SLC22A1 | NM_153187, ENST00000324965 | 2366 | 5916 | 9466 | None |
| Genewiz | TFORF2277 | PRDM2 | NM_001135610, ENST00000376048 | 2367 | 5917 | 9467 | None |
| Genewiz | TFORF2278 | PRDM2 | NM_015866, XM_017002257, ENST00000311066 | 2368 | 5918 | 9468 | None |
| Genewiz | TFORF2279 | PRDM2 | XM_017002256, NM_012231, XM_017002255, | 2369 | 5919 | 9469 | None |
| | | | ENST00000235372 | | | | |
| Genewiz | TFORF2280 | PRDM2 | NM_001007257, XM_017002262, ENST00000413440, | 2370 | 5920 | 9470 | None |
| | | | ENST00000343137 | | | | |
| Genewiz | TFORF2281 | MYBBP1A | NM_001105538, ENST00000381556 | 2371 | 5921 | 9471 | None |
| Genewiz | TFORF2282 | MYBBP1A | NM_014520, ENST00000254718 | 2372 | 5922 | 9472 | None |
| Genewiz | TFORF2283 | HES1 | NM_005524, ENST00000232424 | 2373 | 5923 | 9473 | None |
| Genewiz | TFORF2284 | HES2 | NM_019089, ENST00000377834 | 2374 | 5924 | 9474 | None |
| Genewiz | TFORF2285 | HES3 | NM_001024598, ENST00000377898 | 2375 | 5925 | 9475 | None |
| Genewiz | TFORF2286 | HES4 | XM_005244771, ENST00000484667 | 2376 | 5926 | 9476 | None |
| Genewiz | TFORF2287 | HES4 | NM_001142467, ENST00000428771 | 2377 | 5927 | 9477 | None |
| Genewiz | TFORF2288 | HES4 | NM_021170, ENST00000304952 | 2378 | 5928 | 9478 | None |
| Genewiz | TFORF2289 | HES5 | NM_001010926, ENST00000378453 | 2379 | 5929 | 9479 | None |
| Genewiz | TFORF2290 | HES6 | NM_001142853, ENST00000409002 | 2380 | 5930 | 9480 | None |
| Genewiz | TFORF2291 | HES6 | NM_001282434, ENST00000409574 | 2381 | 5931 | 9481 | None |
| Genewiz | TFORF2292 | HES7 | NM_001165967, ENST00000541682 | 2382 | 5932 | 9482 | None |
| Genewiz | TFORF2293 | HES7 | NM_032580, ENST00000317814 | 2383 | 5933 | 9483 | None |
| Genewiz | TFORF2294 | TAF13 | NM_005645, ENST00000461096, ENST00000338366 | 2384 | 5934 | 9484 | None |
| Genewiz | TFORF2295 | TAF11 | NM_005643, ENST00000361288 | 2385 | 5935 | 9485 | None |
| Genewiz | TFORF2296 | TAF11 | NM_001270488, ENST00000420584 | 2386 | 5936 | 9486 | None |
| Genewiz | TFORF2297 | TAF10 | NM_006284, ENST00000299424 | 2387 | 5937 | 9487 | None |
| Genewiz | TFORF2298 | ZFP69B | XM_005271137, NM_023070, ENST00000411995, | 2388 | 5938 | 9488 | None |
| | | | ENST00000361584 | | | | |
| Genewiz | TFORF2299 | HIC1 | NM_006497, ENST00000399849, ENST00000619757 | 2389 | 5939 | 9489 | None |
| Genewiz | TFORF2300 | HIC1 | NM_001098202, ENST00000322941 | 2390 | 5940 | 9490 | None |
| Genewiz | TFORF2301 | HIC2 | NM_015094, XM_011530010, XM_011530008, | 2391 | 5941 | 9491 | None |
| | | | XM_011530009, XM_011530007, ENST00000407464, | | | | |
| | | | ENST00000407598, ENST00000443632 | | | | |
| Genewiz | TFORF2302 | CIITA | XM_011522493, NM_000246, ENST00000324288 | 2392 | 5942 | 9492 | None |
| Genewiz | TFORF2303 | CIITA | NM_001286403, ENST00000381835 | 2393 | 5943 | 9493 | None |
| Genewiz | TFORF2304 | CIITA | XM_011522492, NM_001286402, ENST00000618327 | 2394 | 5944 | 9494 | None |
| Genewiz | TFORF2305 | ETV1 | NM_001163152, ENST00000399357 | 2395 | 5945 | 9495 | None |
| Genewiz | TFORF2306 | ETV1 | XM_011515167, ENST00000405358 | 2396 | 5946 | 9496 | None |
| Genewiz | TFORF2307 | ETV1 | NM_001163147, ENST00000405192 | 2397 | 5947 | 9497 | None |
| Genewiz | TFORF2308 | ETV1 | NM_001163149, NM_001163148, ENST00000242066, | 2398 | 5948 | 9498 | None |
| | | | ENST00000403685 | | | | |
| Genewiz | TFORF2309 | ETV1 | NM_001163151, ENST00000420159 | 2399 | 5949 | 9499 | None |
| Genewiz | TFORF2310 | ETV1 | NM_001163150, ENST00000403527 | 2400 | 5950 | 9500 | None |
| Genewiz | TFORF2311 | RXRG | NM_001256571, NM_001256570, ENST00000619224 | 2401 | 5951 | 9501 | None |
| Genewiz | TFORF2312 | ETV3 | XM_006711210, NM_001145312, ENST00000368192 | 2402 | 5952 | 9502 | None |
| Genewiz | TFORF2313 | ETV3 | NM_005240, ENST00000326786 | 2403 | 5953 | 9503 | None |
| Genewiz | TFORF2314 | RXRB | NM_001270401, ENST00000374685 | 2404 | 5954 | 9504 | None |
| Genewiz | TFORF2315 | RXRB | NM_021976, ENST00000374680 | 2405 | 5955 | 9505 | None |
| Genewiz | TFORF2316 | ETV4 | NM_001986, NM_001079675, ENST00000319349, | 2406 | 5956 | 9506 | None |
| | | | ENST00000393664, ENST00000591713 | | | | |
| Genewiz | TFORF2317 | ETV4 | NM_001261438, NM_001261437, ENST00000538265, | 2407 | 5957 | 9507 | None |
| | | | ENST00000545954 | | | | |
| Genewiz | TFORF2318 | ZNF732 | NM_001137608, ENST00000419098 | 2408 | 5958 | 9508 | None |
| Genewiz | TFORF2319 | TAF1B | NM_005680, ENST00000263663 | 2409 | 5959 | 9509 | None |
| Genewiz | TFORF2320 | TAF1A | NM_001201536, NM_005681, ENST00000350027, | 2410 | 5960 | 9510 | None |
| | | | ENST00000352967 | | | | |
| Genewiz | TFORF2321 | TAF1A | NM_139352, XM_017002759, XM_017002760, | 2411 | 5961 | 9511 | None |
| | | | XM_006711613, ENST00000366890 | | | | |
| Genewiz | TFORF2322 | ZNF736 | NM_001170905, ENST00000355095, ENST00000423484 | 2412 | 5962 | 9512 | None |
| Genewiz | TFORF2323 | TAF1 | NM_138923, ENST00000373790 | 2413 | 5963 | 9513 | None |
| Genewiz | TFORF2324 | TAF1 | NM_001286074, ENST00000423759 | 2414 | 5964 | 9514 | None |
| Genewiz | TFORF2325 | TAF1 | NM_004606, ENST00000276072 | 2415 | 5965 | 9515 | None |
| Genewiz | TFORF2326 | ZNF735 | NM_001159524, ENST00000429565 | 2416 | 5966 | 9516 | None |
| Genewiz | TFORF2327 | CIZ1 | NM_001257976, ENST00000629610 | 2417 | 5967 | 9517 | None |
| Genewiz | TFORF2328 | CIZ1 | NM_001131015, XM_017014596, XM_005251891, | 2418 | 5968 | 9518 | None |
| | | | ENST00000372948 | | | | |
| Genewiz | TFORF2329 | CIZ1 | NM_001131016, NM_012127, XM_005251888, | 2419 | 5969 | 9519 | None |
| | | | ENST00000372938, ENST00000634901 | | | | |
| Genewiz | TFORF2330 | CIZ1 | NM_001131018, XM_005251893, ENST00000372954 | 2420 | 5970 | 9520 | None |
| Genewiz | TFORF2331 | CIZ1 | NM_001257975, ENST00000538431 | 2421 | 5971 | 9521 | None |
| Genewiz | TFORF2332 | ZNF492 | NM_020855, ENST00000456783 | 2422 | 5972 | 9522 | None |
| Genewiz | TFORF2333 | ZNF491 | XM_005259730, NM_152356, ENST00000323169 | 2423 | 5973 | 9523 | None |
| Genewiz | TFORF2334 | CTNNB1 | NM_001098210, NM_001098209, NM_001904, | 2424 | 5974 | 9524 | None |
| | | | XM_017005738, XM_005264886, ENST00000405570, | | | | |
| | | | ENST00000396183, ENST00000349496, ENST00000396185 | | | | |
| Genewiz | TFORF2335 | CTNNB1 | XM_006712984, XM_006712983, ENST00000453024 | 2425 | 5975 | 9525 | None |
| Genewiz | TFORF2336 | ZNF14 | NM_021030, ENST00000344099 | 2426 | 5976 | 9526 | None |
| Genewiz | TFORF2337 | ZNF18 | XM_017025009, XM_017025010, XM_017025011, | 2427 | 5977 | 9527 | None |
| | | | XM_017025008, NM_001303282, ENST00000454073 | | | | |
| Genewiz | TFORF2338 | ZNF18 | XM_011524002, XM_017025007, XM_017025006, | 2428 | 5978 | 9528 | None |
| | | | NM_144680, NM_001303281, ENST00000580306, | | | | |
| | | | ENST00000580613, ENST00000322748 | | | | |
| Genewiz | TFORF2339 | ZNF19 | NM_006961, ENST00000288177, ENST00000564230 | 2429 | 5979 | 9529 | None |
| Genewiz | TFORF2340 | MITF | NM_006722, ENST00000328528 | 2430 | 5980 | 9530 | None |
| Genewiz | TFORF2341 | MITF | NM_198177, ENST00000314589 | 2431 | 5981 | 9531 | None |
| Genewiz | TFORF2342 | MITF | NM_198159, ENST00000352241 | 2432 | 5982 | 9532 | None |
| Genewiz | TFORF2343 | MITF | XM_005264754, ENST00000448226 | 2433 | 5983 | 9533 | None |
| Genewiz | TFORF2344 | MITF | NM_198178, ENST00000531774 | 2434 | 5984 | 9534 | None |
| Genewiz | TFORF2345 | MITF | XM_017006448, NM_001184967, ENST00000472437 | 2435 | 5985 | 9535 | None |
| Genewiz | TFORF2346 | MITF | NM_000248, ENST00000394351 | 2436 | 5986 | 9536 | None |
| Genewiz | TFORF2347 | HOXD8 | NM_001199746, ENST00000450510 | 2437 | 5987 | 9537 | None |
| Genewiz | TFORF2348 | HOXD8 | NM_001199747, ENST00000429017 | 2438 | 5988 | 9538 | None |
| Genewiz | TFORF2349 | HOXD8 | NM_019558, ENST00000313173 | 2439 | 5989 | 9539 | None |
| Genewiz | TFORF2350 | ZNF16 | NM_001029976, NM_006958, XM_005272341, | 2440 | 5990 | 9540 | None |
| | | | XM_011517298, ENST00000276816, ENST00000611477, | | | | |
| | | | ENST00000394909 | | | | |
| Genewiz | TFORF2351 | ZNF17 | NM_006959, ENST00000601808 | 2441 | 5991 | 9541 | None |
| Genewiz | TFORF2352 | ZNF17 | XM_017027205, ENST00000307658 | 2442 | 5992 | 9542 | None |
| Genewiz | TFORF2353 | ZNF12 | NM_016265, ENST00000405858 | 2443 | 5993 | 9543 | None |
| Genewiz | TFORF2354 | ZNF12 | NM_006956, ENST00000342651 | 2444 | 5994 | 9544 | None |
| Genewiz | TFORF2355 | PROP1 | NM_006261, ENST00000308304 | 2445 | 5995 | 9545 | None |
| Genewiz | TFORF2356 | ZNF648 | XM_017000301, XM_017000302, NM_001009992, | 2446 | 5996 | 9546 | None |
| | | | ENST00000339948 | | | | |
| Genewiz | TFORF2357 | ZNF649 | NM_023074, ENST00000354957 | 2447 | 5997 | 9547 | None |
| Genewiz | TFORF2358 | ZNF644 | NM_201269, XM_017002491, XM_017002488, | 2448 | 5998 | 9548 | None |
| | | | XM_017002489, XM_017002490, ENST00000370440, | | | | |
| | | | ENST00000337393 | | | | |
| Genewiz | TFORF2359 | ZNF644 | NM_016620, NM_032186, XM_017002492, | 2449 | 5999 | 9549 | None |
| | | | XM_017002493, XM_017002494, ENST00000347275, | | | | |
| | | | ENST00000361321 | | | | |
| Genewiz | TFORF2360 | ZNF641 | XM_017018797, NM_001172682, ENST00000448928 | 2450 | 6000 | 9550 | None |
| Genewiz | TFORF2361 | ZNF641 | XM_011537897, NM_152320, ENST00000544117, | 2451 | 6001 | 9551 | None |
| | | | ENST00000301042 | | | | |
| Genewiz | TFORF2362 | ZNF641 | XM_005268638, XM_011537898, NM_001172681, | 2452 | 6002 | 9552 | None |
| | | | ENST00000547026 | | | | |
| Genewiz | TFORF2363 | TONSL | NM_013432, ENST00000409379 | 2453 | 6003 | 9553 | None |
| Genewiz | TFORF2364 | CREB1 | NM_004379, XM_011510647, ENST00000430624, | 2454 | 6004 | 9554 | None |
| | | | ENST00000353267 | | | | |
| Genewiz | TFORF2365 | ZNF398 | NM_020781, XM_011516441, ENST00000426851, | 2455 | 6005 | 9555 | None |
| | | | ENST00000483892, ENST00000491174 | | | | |
| Genewiz | TFORF2366 | MBD4 | NM_001276271, ENST00000507208 | 2456 | 6006 | 9556 | None |
| Genewiz | TFORF2367 | MBD4 | NM_001276272, ENST00000503197 | 2457 | 6007 | 9557 | None |
| Genewiz | TFORF2368 | MBD4 | NM_001276273, ENST00000393278 | 2458 | 6008 | 9558 | None |
| Genewiz | TFORF2369 | MBD4 | NM_003925, ENST00000249910 | 2459 | 6009 | 9559 | None |
| Genewiz | TFORF2370 | MBD4 | NM_001276270, ENST00000429544 | 2460 | 6010 | 9560 | None |
| Genewiz | TFORF2371 | ZNF468 | NM_001008801, ENST00000595646 | 2461 | 6011 | 9561 | None |
| Genewiz | TFORF2372 | ZNF468 | NM_001277120, ENST00000243639 | 2462 | 6012 | 9562 | None |
| Genewiz | TFORF2373 | MBD1 | NM_002384, XM_017025776, ENST00000398488 | 2463 | 6013 | 9563 | None |
| Genewiz | TFORF2374 | MBD1 | NM_001204136, ENST00000590208 | 2464 | 6014 | 9564 | None |
| Genewiz | TFORF2375 | MBD1 | NM_001204151, ENST00000591535 | 2465 | 6015 | 9565 | None |
| Genewiz | TFORF2376 | MBD1 | XM_011525993, XM_011525994, NM_001204137, | 2466 | 6016 | 9566 | None |
| | | | ENST00000585595, ENST00000457839 | | | | |
| Genewiz | TFORF2377 | MBD1 | NM_015844, XM_017025770, XM_017025771, | 2467 | 6017 | 9567 | None |
| | | | ENST00000347968, ENST00000398493 | | | | |
| Genewiz | TFORF2378 | MBD1 | NM_001204142, ENST00000339998 | 2468 | 6018 | 9568 | None |
| Genewiz | TFORF2379 | MBD1 | NM_015847, ENST00000353909 | 2469 | 6019 | 9569 | None |
| Genewiz | TFORF2380 | MBD1 | NM_001204143, XM_011526006, ENST00000587605 | 2470 | 6020 | 9570 | None |
| Genewiz | TFORF2381 | MBD1 | NM_001204139, NM_015846, XM_005258271, | 2471 | 6021 | 9571 | None |
| | | | XM_017025760, ENST00000382948, ENST00000591416, | | | | |
| | | | ENST00000269468 | | | | |
| Genewiz | TFORF2382 | MBD1 | NM_015845, ENST00000269471, ENST00000588937 | 2472 | 6022 | 9572 | None |
| Genewiz | TFORF2383 | MBD1 | NM_001204141, ENST00000585672 | 2473 | 6023 | 9573 | None |
| Genewiz | TFORF2384 | MBD3 | NM_001281454, ENST00000156825 | 2474 | 6024 | 9574 | None |
| Genewiz | TFORF2385 | MBD3 | NM_001281453, ENST00000434436 | 2475 | 6025 | 9575 | None |
| Genewiz | TFORF2386 | MBD2 | NM_015832, ENST00000583046 | 2476 | 6026 | 9576 | None |
| Genewiz | TFORF2387 | MBD2 | NM_003927, ENST00000256429 | 2477 | 6027 | 9577 | None |
| Genewiz | TFORF2388 | ZNF462 | NM_021224, XM_006717216, ENST00000277225 | 2478 | 6028 | 9578 | None |
| Genewiz | TFORF2389 | ZNF391 | NM_001322288, XM_011514569, XM_006715086, | 2479 | 6029 | 9579 | None |
| | | | NM_001322293, NM_001076781, NM_001322289, | | | | |
| | | | XM_005249082, ENST00000244576 | | | | |
| Genewiz | TFORF2390 | ZNF460 | XM_005258422, ENST00000537645 | 2480 | 6030 | 9580 | None |
| Genewiz | TFORF2391 | ZNF461 | NM_001297623, ENST00000360357 | 2481 | 6031 | 9581 | None |
| Genewiz | TFORF2392 | ZNF461 | NM_153257, ENST00000588268 | 2482 | 6032 | 9582 | None |
| Genewiz | TFORF2393 | ZNF394 | XM_017012711, ENST00000426306 | 2483 | 6033 | 9583 | None |
| Genewiz | TFORF2394 | ZNF396 | NM_001322291, ENST00000586687 | 2484 | 6034 | 9584 | None |
| Genewiz | TFORF2395 | ZNF396 | NM_001322290, NM_001322286, XM_006722432, | 2485 | 6035 | 9585 | None |
| | | | XM_017025688, XM_017025687, ENST00000589332 | | | | |
| Genewiz | TFORF2396 | ZNF396 | NM_145756, ENST00000306346 | 2486 | 6036 | 9586 | None |
| Genewiz | TFORF2397 | ZNF397 | XM_017026042, XM_011526231, NM_001135178, | 2487 | 6037 | 9587 | None |
| | | | ENST00000330501 | | | | |
| Genewiz | TFORF2398 | ZFPM2 | NM_012082, ENST00000407775 | 2488 | 6038 | 9588 | None |
| Genewiz | TFORF2399 | ZFPM1 | NM_153813, ENST00000319555 | 2489 | 6039 | 9589 | None |
| Genewiz | TFORF2400 | REXO1 | NM_020695, ENST00000170168 | 2490 | 6040 | 9590 | None |
| Genewiz | TFORF2401 | NFE2L3 | NM_004289, ENST00000056233 | 2491 | 6041 | 9591 | None |
| Genewiz | TFORF2402 | NFE2L2 | NM_001145413, ENST00000446151 | 2492 | 6042 | 9592 | None |
| Genewiz | TFORF2403 | NFE2L2 | NM_001145412, NM_001313901, NM_001313900, | 2493 | 6043 | 9593 | None |
| | | | ENST00000397063, ENST00000464747 | | | | |
| Genewiz | TFORF2404 | NFE2L1 | XM_005257413, XM_005257412, ENST00000585291, | 2494 | 6044 | 9594 | None |
| | | | ENST00000357480 | | | | |
| Genewiz | TFORF2405 | NFE2L1 | XM_017024691, XM_005257411, ENST00000361665 | 2495 | 6045 | 9595 | None |
| Genewiz | TFORF2406 | NFE2L1 | NM_003204, XM_005257410, ENST00000362042 | 2496 | 6046 | 9596 | None |
| Genewiz | TFORF2407 | ZNF264 | NM_003417, ENST00000263095, ENST00000536056 | 2497 | 6047 | 9597 | None |
| Genewiz | TFORF2408 | FOXF1 | NM_001451, ENST00000262426 | 2498 | 6048 | 9598 | None |
| Genewiz | TFORF2409 | FOXF2 | NM_001452, ENST00000259806 | 2499 | 6049 | 9599 | None |
| Genewiz | TFORF2410 | ZNF705G | XM_017012941, NM_001164457, XM_017012942, | 2500 | 6050 | 9600 | None |
| | | | ENST00000400156 | | | | |
| Genewiz | TFORF2411 | KLF3 | NM_016531, ENST00000261438 | 2501 | 6051 | 9601 | None |
| Genewiz | TFORF2412 | ZNF705D | NM_001039615, XM_011543832, ENST00000400085, | 2502 | 6052 | 9602 | None |
| | | | ENST00000400078 | | | | |
| Genewiz | TFORF2413 | ZNF268 | NM_001165887, ENST00000592241 | 2503 | 6053 | 9603 | None |
| Genewiz | TFORF2414 | ZNF268 | NM_152943, ENST00000541009 | 2504 | 6054 | 9604 | None |
| Genewiz | TFORF2415 | ZNF268 | NM_001165884, ENST00000536899 | 2505 | 6055 | 9605 | None |
| Genewiz | TFORF2416 | ZNF268 | NM_003415, NM_001165881, ENST00000536435, | 2506 | 6056 | 9606 | None |
| | | | ENST00000228289 | | | | |
| Genewiz | TFORF2417 | ZNF268 | NM_001165885, ENST00000541211 | 2507 | 6057 | 9607 | None |
| Genewiz | TFORF2418 | ZNF268 | NM_001165883, ENST00000539248 | 2508 | 6058 | 9608 | None |
| Genewiz | TFORF2419 | ZNF268 | NM_001165886, ENST00000542711 | 2509 | 6059 | 9609 | None |
| Genewiz | TFORF2420 | ZNF705B | NM_001193630, ENST00000400120 | 2510 | 6060 | 9610 | None |
| Genewiz | TFORF2421 | RAX | NM_013435, ENST00000334889 | 2511 | 6061 | 9611 | None |
| Genewiz | TFORF2422 | RBPJL | NM_001281449, ENST00000372743 | 2512 | 6062 | 9612 | None |
| Genewiz | TFORF2423 | RBPJL | NM_001281448, ENST00000372741 | 2513 | 6063 | 9613 | None |
| Genewiz | TFORF2424 | RBPJL | NM_014276, ENST00000343694 | 2514 | 6064 | 9614 | None |
| Genewiz | TFORF2425 | ZNF646 | XM_011545990, XM_005255711, XM_005255710, | 2515 | 6065 | 9615 | None |
| | | | NM_014699, ENST00000300850 | | | | |
| Genewiz | TFORF2426 | ZNF841 | XM_017026656, XM_017026657, NM_001136499, | 2516 | 6066 | 9616 | None |
| | | | ENST00000389534, ENST00000594295 | | | | |
| Genewiz | TFORF2427 | ZNF841 | NM_001321349, ENST00000426391 | 2517 | 6067 | 9617 | None |
| Genewiz | TFORF2428 | ARID1B | XM_005267069, ENST00000636930 | 2518 | 6068 | 9618 | None |
| Genewiz | TFORF2429 | ARID1B | NM_017519, ENST00000350026 | 2519 | 6069 | 9619 | None |
| Genewiz | TFORF2430 | ARID1B | NM_020732, ENST00000346085 | 2520 | 6070 | 9620 | None |
| Genewiz | TFORF2431 | ARID1B | XM_017011109, ENST00000637904 | 2521 | 6071 | 9621 | None |
| Genewiz | TFORF2432 | ARID1A | NM_006015, ENST00000324856 | 2522 | 6072 | 9622 | None |
| Genewiz | TFORF2433 | ARID1A | NM_139135, ENST00000457599 | 2523 | 6073 | 9623 | None |
| Genewiz | TFORF2434 | ZNF280D | NM_001288589, NM_001002844, ENST00000558320 | 2524 | 6074 | 9624 | None |
| Genewiz | TFORF2435 | ZNF280D | NM_001002843, ENST00000559237 | 2525 | 6075 | 9625 | None |
| Genewiz | TFORF2436 | ZNF280D | XM_005254483, ENST00000559000 | 2526 | 6076 | 9626 | None |
| Genewiz | TFORF2437 | ZNF280D | NM_017661, NM_001288588, XM_017022344, | 2527 | 6077 | 9627 | None |
| | | | XM_017022345, XM_011521702, ENST00000267807 | | | | |
| Genewiz | TFORF2438 | ZNF280B | NM_080764, XM_011529893, XM_011529895, | 2528 | 6078 | 9628 | None |
| | | | XM_011529896, XM_011529897, ENST00000626650, | | | | |
| | | | ENST00000619852, ENST00000613655 | | | | |
| Genewiz | TFORF2439 | YEATS4 | NM_001300950, ENST00000548020 | 2529 | 6079 | 9629 | None |
| Genewiz | TFORF2440 | FUS | NM_001170634, ENST00000380244 | 2530 | 6080 | 9630 | None |
| Genewiz | TFORF2441 | FUS | NM_004960, ENST00000254108 | 2531 | 6081 | 9631 | None |
| Genewiz | TFORF2442 | ABL1 | NM_005157, ENST00000318560 | 2532 | 6082 | 9632 | None |
| Genewiz | TFORF2443 | ABL1 | NM_007313, ENST00000372348 | 2533 | 6083 | 9633 | None |
| Genewiz | TFORF2444 | SMARCE1 | NM_003079, ENST00000348513 | 2534 | 6084 | 9634 | None |
| Genewiz | TFORF2445 | ZIK1 | NM_001010879, ENST00000597850 | 2535 | 6085 | 9635 | None |
| Genewiz | TFORF2446 | ZIK1 | NM_001321146, XM_011526766, ENST00000599456 | 2536 | 6086 | 9636 | None |
| Genewiz | TFORF2447 | ZIK1 | NM_001321145, ENST00000536878 | 2537 | 6087 | 9637 | None |
| Genewiz | TFORF2448 | GBX2 | NM_001301687, ENST00000551105 | 2538 | 6088 | 9638 | None |
| Genewiz | TFORF2449 | GBX1 | NM_001098834, ENST00000297537 | 2539 | 6089 | 9639 | None |
| Genewiz | TFORF2450 | MLLT10 | NM_001324296, NM_001195628, NM_001195630, | 2540 | 6090 | 9640 | None |
| | | | ENST00000377100 | | | | |
| Genewiz | TFORF2451 | MLLT10 | NM_004641, ENST00000377072 | 2541 | 6091 | 9641 | None |
| Genewiz | TFORF2452 | MLLT10 | NM_001195626, ENST00000631589, ENST00000307729, | 2542 | 6092 | 9642 | None |
| | | | ENST00000377059 | | | | |
| Genewiz | TFORF2453 | ZNF845 | NM_138374, NM_001321523, NM_001321524, | 2543 | 6093 | 9643 | None |
| | | | NM_001321522, ENST00000458035, ENST00000595091 | | | | |
| Genewiz | TFORF2454 | IRF3 | NM_001197125, NM_001197126, ENST00000377135, | 2544 | 6094 | 9644 | None |
| | | | ENST00000593922, ENST00000599144, ENST00000598808 | | | | |
| Genewiz | TFORF2455 | IRF3 | NM_001197127, NM_001197128, ENST00000596765, | 2545 | 6095 | 9645 | None |
| | | | ENST00000600022 | | | | |
| Genewiz | TFORF2456 | IRF3 | NM_001197124, ENST00000599223 | 2546 | 6096 | 9646 | None |
| Genewiz | TFORF2457 | IRF3 | NM_001571, XM_017026767, XM_017026766, | 2547 | 6097 | 9647 | None |
| | | | ENST00000309877, ENST00000377139, ENST00000597198 | | | | |
| Genewiz | TFORF2458 | IRF3 | NM_001197122, XM_006723198, XM_006723197, | 2548 | 6098 | 9648 | None |
| | | | ENST00000601291 | | | | |
| Genewiz | TFORF2459 | IRF1 | NM_002198, ENST00000245414, ENST00000405885 | 2549 | 6099 | 9649 | None |
| Genewiz | TFORF2460 | IRF7 | NM_004029, ENST00000348655 | 2550 | 6100 | 9650 | None |
| Genewiz | TFORF2461 | IRF7 | NM_004031, XM_005252906, ENST00000397566, | 2551 | 6101 | 9651 | None |
| | | | ENST00000330243 | | | | |
| Genewiz | TFORF2462 | IRF7 | NM_001572, ENST00000397574 | 2552 | 6102 | 9652 | None |
| Genewiz | TFORF2463 | IRF7 | XM_005252909, ENST00000397570 | 2553 | 6103 | 9653 | None |
| Genewiz | TFORF2464 | IRF6 | NM_001206696, ENST00000542854 | 2554 | 6104 | 9654 | None |
| Genewiz | TFORF2465 | IRF6 | NM_006147, ENST00000367021 | 2555 | 6105 | 9655 | None |
| Genewiz | TFORF2466 | IRF5 | NM_001242452, ENST00000477535 | 2556 | 6106 | 9656 | None |
| Genewiz | TFORF2467 | IRF5 | XM_011516160, NM_001098629, XM_006715974, | 2557 | 6107 | 9657 | None |
| | | | XM_011516158, XM_011516159, XM_005250317, | | | | |
| | | | ENST00000489702, ENST00000357234 | | | | |
| Genewiz | TFORF2468 | LZTR1 | NM_006767, ENST00000215739 | 2558 | 6108 | 9658 | None |
| Genewiz | TFORF2469 | IRF8 | XM_017023199, ENST00000562492 | 2559 | 6109 | 9659 | None |
| Genewiz | TFORF2470 | PDX1 | NM_000209, ENST00000381033 | 2560 | 6110 | 9660 | None |
| Genewiz | TFORF2471 | HDAC5 | NM_005474, XM_005256906, ENST00000586802 | 2561 | 6111 | 9661 | None |
| Genewiz | TFORF2472 | HDAC5 | NM_001015053, ENST00000225983 | 2562 | 6112 | 9662 | None |
| Genewiz | TFORF2473 | HDAC4 | NM_006037, ENST00000345617 | 2563 | 6113 | 9663 | None |
| Genewiz | TFORF2474 | ONECUT3 | NM_001080488, ENST00000382349 | 2564 | 6114 | 9664 | None |
| Genewiz | TFORF2475 | ZFHX3 | NM_001164766, ENST00000397992 | 2565 | 6115 | 9665 | None |
| Genewiz | TFORF2476 | ZFHX2 | NM_033400, XM_011537247, XM_011537246, | 2566 | 6116 | 9666 | None |
| | | | XM_017021714, XM_011537245, ENST00000419474 | | | | |
| Genewiz | TFORF2477 | OXSR1 | NM_005109, ENST00000311806 | 2567 | 6117 | 9667 | None |
| Genewiz | TFORF2478 | MESP1 | NM_018670, ENST00000300057 | 2568 | 6118 | 9668 | None |
| Genewiz | TFORF2479 | MESP2 | NM_001039958, ENST00000341735 | 2569 | 6119 | 9669 | None |
| Genewiz | TFORF2480 | MXD4 | XM_017007656, ENST00000510822 | 2570 | 6120 | 9670 | None |
| Genewiz | TFORF2481 | MXD4 | NM_006454, ENST00000337190 | 2571 | 6121 | 9671 | None |
| Genewiz | TFORF2482 | MXD3 | NM_001142935, ENST00000427908 | 2572 | 6122 | 9672 | None |
| Genewiz | TFORF2483 | CBX2 | NM_005189, ENST00000310942 | 2573 | 6123 | 9673 | None |
| Genewiz | TFORF2484 | CBX2 | NM_032647, ENST00000269399 | 2574 | 6124 | 9674 | None |
| Genewiz | TFORF2485 | MXD1 | NM_001202514, ENST00000540449 | 2575 | 6125 | 9675 | None |
| Genewiz | TFORF2486 | ZBTB11 | NM_014415, ENST00000312938 | 2576 | 6126 | 9676 | None |
| Genewiz | TFORF2487 | ZBTB10 | NM_001277145, ENST00000379091 | 2577 | 6127 | 9677 | None |
| Genewiz | TFORF2488 | ZBTB10 | NM_001105539, ENST00000430430, ENST00000455036 | 2578 | 6128 | 9678 | None |
| Genewiz | TFORF2489 | ZBTB10 | NM_023929, ENST00000426744 | 2579 | 6129 | 9679 | None |
| Genewiz | TFORF2490 | ZBTB17 | NM_003443, ENST00000375743 | 2580 | 6130 | 9680 | None |
| Genewiz | TFORF2491 | ZBTB17 | NM_001242884, XM_011542088, ENST00000537142 | 2581 | 6131 | 9681 | None |
| Genewiz | TFORF2492 | ZBTB17 | NM_001287603, ENST00000375733 | 2582 | 6132 | 9682 | None |
| Genewiz | TFORF2493 | ZBTB16 | NM_006006, XM_017018258, XM_017018257, | 2583 | 6133 | 9683 | None |
| | | | NM_001018011, XM_017018259, ENST00000335953, | | | | |
| | | | ENST00000392996 | | | | |
| Genewiz | TFORF2494 | ZBTB18 | NM_001278196, XM_017000060, XM_005273006, | 2584 | 6134 | 9684 | None |
| | | | NM_006352, ENST00000622512 | | | | |
| Genewiz | TFORF2495 | ZBTB18 | NM_205768, ENST00000358704 | 2585 | 6135 | 9685 | None |
| Genewiz | TFORF2496 | ASCL3 | NM_020646, ENST00000531618 | 2586 | 6136 | 9686 | None |
| Genewiz | TFORF2497 | ASCL1 | NM_004316, ENST00000266744 | 2587 | 6137 | 9687 | None |
| Genewiz | TFORF2498 | ASCL4 | NM_203436, ENST00000342331 | 2588 | 6138 | 9688 | None |
| Genewiz | TFORF2499 | ASCL5 | NM_001270601, ENST00000458416, ENST00000449188 | 2589 | 6139 | 9689 | None |
| Genewiz | TFORF2500 | PML | NM_033249, ENST00000564428 | 2590 | 6140 | 9690 | None |
| Genewiz | TFORF2501 | PML | NM_033238, ENST00000268058 | 2591 | 6141 | 9691 | None |
| Genewiz | TFORF2502 | PML | NM_033244, ENST00000569965, ENST00000436891 | 2592 | 6142 | 9692 | None |
| Genewiz | TFORF2503 | PML | NM_033240, ENST00000435786 | 2593 | 6143 | 9693 | None |
| Genewiz | TFORF2504 | PML | NM_033250, ENST00000354026 | 2594 | 6144 | 9694 | None |
| Genewiz | TFORF2505 | PML | NM_033246, ENST00000567543, ENST00000359928 | 2595 | 6145 | 9695 | None |
| Genewiz | TFORF2506 | PML | NM_002675, ENST00000395135 | 2596 | 6146 | 9696 | None |
| Genewiz | TFORF2507 | PML | NM_033239, ENST00000268059 | 2597 | 6147 | 9697 | None |
| Genewiz | TFORF2508 | PML | NM_033247, ENST00000395132 | 2598 | 6148 | 9698 | None |
| Genewiz | TFORF2509 | EP300 | NM_001429, ENST00000263253 | 2599 | 6149 | 9699 | None |
| Genewiz | TFORF2510 | NAT10 | NM_001144030, ENST00000531159 | 2600 | 6150 | 9700 | None |
| Genewiz | TFORF2511 | PDCD2 | NM_001199461, ENST00000614056 | 2601 | 6151 | 9701 | None |
| Genewiz | TFORF2512 | PDCD2 | NM_001199463, ENST00000443345 | 2602 | 6152 | 9702 | None |
| Genewiz | TFORF2513 | PDCD2 | NM_001199464, ENST00000537445 | 2603 | 6153 | 9703 | None |
| Genewiz | TFORF2514 | PDCD2 | NM_002598, ENST00000541970 | 2604 | 6154 | 9704 | None |
| Genewiz | TFORF2515 | PDCD2 | NM_001199462, ENST00000392090 | 2605 | 6155 | 9705 | None |
| Genewiz | TFORF2516 | PDCD2 | NM_144781, ENST00000453163 | 2606 | 6156 | 9706 | None |
| Genewiz | TFORF2517 | PDCD2 | XM_017010926, ENST00000542896 | 2607 | 6157 | 9707 | None |
| Genewiz | TFORF2518 | ZFP37 | NM_003408, ENST00000374227 | 2608 | 6158 | 9708 | None |
| Genewiz | TFORF2519 | ZFP37 | NM_001282518, ENST00000555206 | 2609 | 6159 | 9709 | None |
| Genewiz | TFORF2520 | ZFP37 | NM_001282515, ENST00000553380 | 2610 | 6160 | 9710 | None |
| Genewiz | TFORF2521 | AATF | NM_012138, ENST00000619387 | 2611 | 6161 | 9711 | None |
| Genewiz | TFORF2522 | ZNF853 | NM_017560, ENST00000457543 | 2612 | 6162 | 9712 | None |
| Genewiz | TFORF2523 | NRF1 | NM_005011, NM_001040110, ENST00000393232, | 2613 | 6163 | 9713 | None |
| | | | ENST00000223190, ENST00000393230 | | | | |
| Genewiz | TFORF2524 | ERCC8 | NM_000082, ENST00000265038 | 2614 | 6164 | 9714 | None |
| Genewiz | TFORF2525 | ERCC2 | NM_001130867, ENST00000485403 | 2615 | 6165 | 9715 | None |
| Genewiz | TFORF2526 | ERCC2 | NM_000400, ENST00000391945 | 2616 | 6166 | 9716 | None |
| Genewiz | TFORF2527 | ERCC3 | NM_000122, ENST00000285398 | 2617 | 6167 | 9717 | None |
| Genewiz | TFORF2528 | ERCC6 | NM_000124, ENST00000355832 | 2618 | 6168 | 9718 | None |
| Genewiz | TFORF2529 | ERCC6 | NM_001277058, NM_001277059, ENST00000447839, | 2619 | 6169 | 9719 | None |
| | | | ENST00000515869 | | | | |
| Genewiz | TFORF2530 | SOX8 | NM_014587, ENST00000293894 | 2620 | 6170 | 9720 | None |
| Genewiz | TFORF2531 | SOX9 | NM_000346, ENST00000245479 | 2621 | 6171 | 9721 | None |
| Genewiz | TFORF2532 | SOX3 | NM_005634, ENST00000370536 | 2622 | 6172 | 9722 | None |
| Genewiz | TFORF2533 | SOX1 | NM_005986, ENST00000330949 | 2623 | 6173 | 9723 | None |
| Genewiz | TFORF2534 | SOX6 | NM_001145811, ENST00000528252 | 2624 | 6174 | 9724 | None |
| Genewiz | TFORF2535 | SOX6 | NM_017508, ENST00000527619 | 2625 | 6175 | 9725 | None |
| Genewiz | TFORF2536 | SOX6 | NM_033326, ENST00000316399, ENST00000396356 | 2626 | 6176 | 9726 | None |
| Genewiz | TFORF2537 | SOX7 | NM_031439, ENST00000304501 | 2627 | 6177 | 9727 | None |
| Genewiz | TFORF2538 | SOX4 | NM_003107, ENST00000244745 | 2628 | 6178 | 9728 | None |
| Genewiz | TFORF2539 | SOX5 | NM_178010, ENST00000396007 | 2629 | 6179 | 9729 | None |
| Genewiz | TFORF2540 | SOX5 | NM_001261415, ENST00000545921 | 2630 | 6180 | 9730 | None |
| Genewiz | TFORF2541 | SOX5 | NM_006940, ENST00000451604 | 2631 | 6181 | 9731 | None |
| Genewiz | TFORF2542 | SOX5 | XM_011520831, ENST00000537393 | 2632 | 6182 | 9732 | None |
| Genewiz | TFORF2543 | SOX5 | XM_017019896, NM_152989, ENST00000546136 | 2633 | 6183 | 9733 | None |
| Genewiz | TFORF2544 | ZNF721 | NM_133474, ENST00000511833 | 2634 | 6184 | 9734 | None |
| Genewiz | TFORF2545 | CEBPB | NM_005194, ENST00000303004 | 2635 | 6185 | 9735 | None |
| Genewiz | TFORF2546 | CEBPA | NM_004364, ENST00000498907 | 2636 | 6186 | 9736 | None |
| Genewiz | TFORF2547 | CEBPD | NM_005195, ENST00000408965 | 2637 | 6187 | 9737 | None |
| Genewiz | TFORF2548 | ZNF729 | NM_001242680, ENST00000601693 | 2638 | 6188 | 9738 | None |
| Genewiz | TFORF2549 | SKI | NM_003036, ENST00000378536 | 2639 | 6189 | 9739 | None |
| Genewiz | TFORF2550 | BID | NM_197966, ENST00000317361 | 2640 | 6190 | 9740 | None |
| Genewiz | TFORF2551 | BID | NM_001244572, NM_001244569, NM_197967, | 2641 | 6191 | 9741 | None |
| | | | NM_001244570, ENST00000615414, ENST00000399767, | | | | |
| | | | ENST00000614949, ENST00000399765, ENST00000611040 | | | | |
| Genewiz | TFORF2552 | BID | NM_001196, NM_001244567, ENST00000622694, | 2642 | 6192 | 9742 | None |
| | | | ENST00000399774, ENST00000551952 | | | | |
| Genewiz | TFORF2553 | AR | NM_001011645, ENST00000396043 | 2643 | 6193 | 9743 | None |
| Genewiz | TFORF2554 | AR | NM_000044, ENST00000374690 | 2644 | 6194 | 9744 | None |
| Genewiz | TFORF2555 | SUZ12 | NM_001321207, ENST00000580398 | 2645 | 6195 | 9745 | None |
| Genewiz | TFORF2556 | ADNP2 | NM_014913, XM_005266656, ENST00000262198 | 2646 | 6196 | 9746 | None |
| Genewiz | TFORF2557 | ZNF69 | NM_021915, ENST00000340180 | 2647 | 6197 | 9747 | None |
| Genewiz | TFORF2558 | TLX1 | NM_005521, ENST00000370196 | 2648 | 6198 | 9748 | None |
| Genewiz | TFORF2559 | TLX1 | NM_001195517, ENST00000467928 | 2649 | 6199 | 9749 | None |
| Genewiz | TFORF2560 | ZNF658 | NM_033160, NM_001317916, XM_005272515, | 2650 | 6200 | 9750 | None |
| | | | XM_017014614, XM_011545679, ENST00000621410, | | | | |
| | | | ENST00000612867 | | | | |
| Genewiz | TFORF2561 | ZNF655 | NM_001085366, NM_001009958, ENST00000357864, | 2651 | 6201 | 9751 | None |
| | | | ENST00000440391 | | | | |
| Genewiz | TFORF2562 | ZNF655 | NM_138494, XM_017012607, XM_017012606, | 2652 | 6202 | 9752 | None |
| | | | NM_001009960, ENST00000252713, ENST00000394163 | | | | |
| Genewiz | TFORF2563 | ZNF655 | XM_017012613, ENST00000626122, ENST00000454654, | 2653 | 6203 | 9753 | None |
| | | | ENST00000425063 | | | | |
| Genewiz | TFORF2564 | ZNF655 | NM_024061, NM_001085367, ENST00000320583 | 2654 | 6204 | 9754 | None |
| Genewiz | TFORF2565 | ZNF655 | XM_017012603, NM_001083956, XM_017012604, | 2655 | 6205 | 9755 | None |
| | | | NM_001085368, ENST00000493277, ENST00000424881 | | | | |
| Genewiz | TFORF2566 | ZNF654 | XM_017006790, ENST00000636215 | 2656 | 6206 | 9756 | None |
| Genewiz | TFORF2567 | ZNF654 | NM_018293, ENST00000309495 | 2657 | 6207 | 9757 | None |
| Genewiz | TFORF2568 | TGIF1 | NM_170695, ENST00000330513 | 2658 | 6208 | 9758 | None |
| Genewiz | TFORF2569 | TGIF1 | NM_174886, NM_001278686, XM_017025959, | 2659 | 6209 | 9759 | None |
| | | | NM_173209, XM_011525735, NM_173210, | | | | |
| | | | NM_173211, ENST00000401449, ENST00000548489, | | | | |
| | | | ENST00000405385, ENST00000551541, ENST00000345133, | | | | |
| | | | ENST0400167, ENST00000472042 | | | | |
| Genewiz | TFORF2570 | TGIF1 | NM_173207, ENST00000618001 | 2660 | 6210 | 9760 | None |
| Genewiz | TFORF2571 | ZNF652 | NM_001145365, NM_014897, ENST00000362063, | 2661 | 6211 | 9761 | None |
| | | | ENST00000430262 | | | | |
| Genewiz | TFORF2572 | ZNF652 | XM_005257166, ENST00000508237 | 2662 | 6212 | 9762 | None |
| Genewiz | TFORF2573 | HSF2 | NM_004506, ENST00000368455 | 2663 | 6213 | 9763 | None |
| Genewiz | TFORF2574 | HSF2 | NM_001135564, ENST00000452194 | 2664 | 6214 | 9764 | None |
| Genewiz | TFORF2575 | HSF5 | NM_001080439, ENST00000323777 | 2665 | 6215 | 9765 | None |
| Genewiz | TFORF2576 | HSF4 | NM_001040667, ENST00000521374 | 2666 | 6216 | 9766 | None |
| Genewiz | TFORF2577 | HSF4 | NM_001538, ENST00000584272 | 2667 | 6217 | 9767 | None |
| Genewiz | TFORF2578 | MEIS3 | NM_020160, ENST00000561293, ENST00000559524 | 2668 | 6218 | 9768 | None |
| Genewiz | TFORF2579 | MEIS3 | XM_017027011, ENST00000561096 | 2669 | 6219 | 9769 | None |
| Genewiz | TFORF2580 | MEIS3 | NM_001009813, ENST00000441740 | 2670 | 6220 | 9770 | None |
| Genewiz | TFORF2581 | MEIS3 | NM_001301059, ENST00000558555 | 2671 | 6221 | 9771 | None |
| Genewiz | TFORF2582 | NR1D1 | NM_021724, ENST00000246672 | 2672 | 6222 | 9772 | None |
| Genewiz | TFORF2583 | MIXL1 | NM_001282402, ENST00000542034 | 2673 | 6223 | 9773 | None |
| Genewiz | TFORF2584 | MIXL1 | NM_031944, ENST00000366810 | 2674 | 6224 | 9774 | None |
| Genewiz | TFORF2585 | CERS6 | NM_203463, ENST00000305747 | 2675 | 6225 | 9775 | None |
| Genewiz | TFORF2586 | CERS4 | XM_011528290, XM_011528293, XM_017027304, | 2676 | 6226 | 9776 | None |
| | | | XM_011528292, XM_{——}017027303, XM_011528294, | | | | |
| | | | NM_024552, XM_017027305, XM_011528295, | | | | |
| | | | XM_011528291, ENST00000251363, ENST00000559450 | | | | |
| Genewiz | TFORF2587 | CERS5 | NM_147190, ENST00000317551 | 2677 | 6227 | 9777 | None |
| Genewiz | TFORF2588 | CERS5 | NM_001281731, ENST00000422340 | 2678 | 6228 | 9778 | None |
| Genewiz | TFORF2589 | ZNF354A | NM_005649, XM_017009791, ENST00000335815 | 2679 | 6229 | 9779 | None |
| Genewiz | TFORF2590 | CERS3 | NM_001290342, NM_001290343, NM_178842, | 2680 | 6230 | 9780 | None |
| | | | XM_017022003, XM_017022004, ENST00000284382, | | | | |
| | | | ENST00000538112, ENST00000394113 | | | | |
| Genewiz | TFORF2591 | ZNF354C | NM_014594, XM_017009409, ENST00000315475 | 2681 | 6231 | 9781 | None |
| Genewiz | TFORF2592 | ZNF354B | NM_058230, ENST00000322434 | 2682 | 6232 | 9782 | None |
| Genewiz | TFORF2593 | BHLHE40 | NM_003670, ENST00000256495 | 2683 | 6233 | 9783 | None |
| Genewiz | TFORF2594 | BHLHE41 | NM_030762, ENST00000242728 | 2684 | 6234 | 9784 | None |
| Genewiz | TFORF2595 | RORC | NM_001001523, ENST00000356728 | 2685 | 6235 | 9785 | None |
| Genewiz | TFORF2596 | RORB | NM_006914, ENST00000376896 | 2686 | 6236 | 9786 | None |
| Genewiz | TFORF2597 | RORA | NM_134260, ENST00000261523 | 2687 | 6237 | 9787 | None |
| Genewiz | TFORF2598 | RORA | NM_134262, ENST00000449337 | 2688 | 6238 | 9788 | None |
| Genewiz | TFORF2599 | RORA | NM_002943, ENST00000309157 | 2689 | 6239 | 9789 | None |
| Genewiz | TFORF2600 | POU6F2 | NM_001166018, ENST00000518318 | 2690 | 6240 | 9790 | None |
| Genewiz | TFORF2601 | POU6F2 | NM_007252, ENST00000403058 | 2691 | 6241 | 9791 | None |
| Genewiz | TFORF2602 | POU6F1 | XM_017019514, XM_017019515, ENST00000333640 | 2692 | 6242 | 9792 | None |
| Genewiz | TFORF2603 | ZNF471 | NM_020813, XM_011527148, ENST00000308031 | 2693 | 6243 | 9793 | None |
| Genewiz | TFORF2604 | ZNF470 | NM_001001668, ENST00000330619, ENST00000391709 | 2694 | 6244 | 9794 | None |
| Genewiz | TFORF2605 | MYRF | NM_001127392, ENST00000278836 | 2695 | 6245 | 9795 | None |
| Genewiz | TFORF2606 | MYRF | NM_013279, ENST00000265460 | 2696 | 6246 | 9796 | None |
| Genewiz | TFORF2607 | DMTF1 | NM_001142326, ENST00000432937 | 2697 | 6247 | 9797 | None |
| Genewiz | TFORF2608 | DMTF1 | NM_001142327, XM_011516737, NM_021145, | 2698 | 6248 | 9798 | None |
| | | | XM_011516735, XM_017012865, ENST00000331242, | | | | |
| | | | ENST00000394703 | | | | |
| Genewiz | TFORF2609 | FOXA2 | NM_153675, ENST00000377115 | 2699 | 6249 | 9799 | None |
| Genewiz | TFORF2610 | FOXA2 | NM_021784, ENST00000419308 | 2700 | 6250 | 9800 | None |
| Genewiz | TFORF2611 | ZNF277 | NM_021994, ENST00000361822 | 2701 | 6251 | 9801 | None |
| Genewiz | TFORF2612 | ZNF276 | NM_001113525, ENST00000443381 | 2702 | 6252 | 9802 | None |
| Genewiz | TFORF2613 | ZNF276 | NM_152287, ENST00000289816 | 2703 | 6253 | 9803 | None |
| Genewiz | TFORF2614 | ZNF275 | NM_001080485, ENST00000370251 | 2704 | 6254 | 9804 | None |
| Genewiz | TFORF2615 | ZNF274 | NM_133502, ENST00000617501, ENST00000610905 | 2705 | 6255 | 9805 | None |
| Genewiz | TFORF2616 | ZNF274 | NM_016324, XM_017026174, XM_011526327, | 2706 | 6256 | 9806 | None |
| | | | ENST00000424679 | | | | |
| Genewiz | TFORF2617 | ZNF274 | NM_001278734, ENST00000326804 | 2707 | 6257 | 9807 | None |
| Genewiz | TFORF2618 | ZNF274 | NM_016325, ENST00000345813 | 2708 | 6258 | 9808 | None |
| Genewiz | TFORF2619 | ZXDB | NM_007157, ENST00000374888 | 2709 | 6259 | 9809 | None |
| Genewiz | TFORF2620 | CRX | NM_000554, ENST00000221996, ENST00000539067 | 2710 | 6260 | 9810 | None |
| Genewiz | TFORF2621 | ZXDA | NM_007156, ENST00000358697 | 2711 | 6261 | 9811 | None |
| Genewiz | TFORF2622 | CRTC1 | NM_015321, ENST00000321949 | 2712 | 6262 | 9812 | None |
| Genewiz | TFORF2623 | CRTC1 | NM_001098482, ENST00000338797 | 2713 | 6263 | 9813 | None |
| Genewiz | TFORF2624 | CRTC3 | NM_001042574, ENST00000420329 | 2714 | 6264 | 9814 | None |
| Genewiz | TFORF2625 | CRTC3 | NM_022769, ENST00000268184 | 2715 | 6265 | 9815 | None |
| Genewiz | TFORF2626 | MAML1 | NM_014757, ENST00000292599 | 2716 | 6266 | 9816 | None |
| Genewiz | TFORF2627 | NONO | NM_001145410, ENST00000535149 | 2717 | 6267 | 9817 | None |
| Genewiz | TFORF2628 | NONO | NM_001145409, NM_007363, NM_001145408, | 2718 | 6268 | 9818 | None |
| | | | ENST00000276079, ENST00000373856, ENST00000373841 | | | | |
| Genewiz | TFORF2629 | INSM2 | NM_032594, ENST00000307169 | 2719 | 6269 | 9819 | None |
| Genewiz | TFORF2630 | ZNF583 | NM_001159860, XM_017026350, NM_152478, | 2720 | 6270 | 9820 | None |
| | | | NM_001159861, XM_011526517, ENST00000291598, | | | | |
| | | | ENST00000333201 | | | | |
| Genewiz | TFORF2631 | INSM1 | NM_002196, ENST00000310227 | 2721 | 6271 | 9821 | None |
| Genewiz | TFORF2632 | TFDP3 | NM_016521, ENST00000310125 | 2722 | 6272 | 9822 | None |
| Genewiz | TFORF2633 | TFDP2 | NM_001178141, ENST00000495310 | 2723 | 6273 | 9823 | None |
| Genewiz | TFORF2634 | TFDP2 | NM_001178142, ENST00000477292 | 2724 | 6274 | 9824 | None |
| Genewiz | TFORF2635 | TFDP2 | XM_017007099, XM_017007097, ENST00000479040 | 2725 | 6275 | 9825 | None |
| Genewiz | TFORF2636 | TFDP2 | NM_001178139, XM_017007091, ENST00000489671 | 2726 | 6276 | 9826 | None |
| Genewiz | TFORF2637 | TFDP2 | NM_001178140, ENST00000499676 | 2727 | 6277 | 9827 | None |
| Genewiz | TFORF2638 | TFDP1 | XM_005268327, NM_007111, ENST00000375370 | 2728 | 6278 | 9828 | None |
| Genewiz | TFORF2639 | CIC | XM_005258673, ENST00000160740 | 2729 | 6279 | 9829 | None |
| Genewiz | TFORF2640 | CIC | NM_015125, ENST00000575354 | 2730 | 6280 | 9830 | None |
| Genewiz | TFORF2641 | PAX3 | NM_181459, ENST00000392069 | 2731 | 6281 | 9831 | None |
| Genewiz | TFORF2642 | PAX3 | NM_181457, ENST00000350526 | 2732 | 6282 | 9832 | None |
| Genewiz | TFORF2643 | PAX3 | NM_181461, ENST00000344493 | 2733 | 6283 | 9833 | None |
| Genewiz | TFORF2644 | PAX3 | NM_013942, ENST00000258387 | 2734 | 6284 | 9834 | None |
| Genewiz | TFORF2645 | PAX3 | NM_001127366, ENST00000409551 | 2735 | 6285 | 9835 | None |
| Genewiz | TFORF2646 | PAX3 | NM_181458, ENST00000392070 | 2736 | 6286 | 9836 | None |
| Genewiz | TFORF2647 | PAX3 | NM_181460, ENST00000336840 | 2737 | 6287 | 9837 | None |
| Genewiz | TFORF2648 | PAX3 | NM_000438, ENST00000409828 | 2738 | 6288 | 9838 | None |
| Genewiz | TFORF2649 | ZNF586 | NM_017652, ENST00000396154 | 2739 | 6289 | 9839 | None |
| Genewiz | TFORF2650 | ZNF586 | NM_001204814, ENST00000391702 | 2740 | 6290 | 9840 | None |
| Genewiz | TFORF2651 | TERB1 | XM_011523008, XM_011523005, XM_011523009, | 2741 | 6291 | 9841 | None |
| | | | XM_011523006, XM_011523007, XM_011523004, | | | | |
| | | | NM_001136505, ENST00000558713, ENST00000433154 | | | | |
| Genewiz | TFORF2652 | ARNTL2 | NM_020183, ENST00000266503 | 2742 | 6292 | 9842 | None |
| Genewiz | TFORF2653 | ARNTL2 | NM_001248002, ENST00000311001 | 2743 | 6293 | 9843 | None |
| Genewiz | TFORF2654 | ARNTL2 | NM_001248005, ENST00000546179 | 2744 | 6294 | 9844 | None |
| Genewiz | TFORF2655 | STAT6 | NM_001178080, NM_001178081, ENST00000538913, | 2745 | 6295 | 9845 | None |
| | | | ENST00000537215 | | | | |
| Genewiz | TFORF2656 | STAT3 | XM_011525146, NM_213662, XM_017024972, | 2746 | 6296 | 9846 | None |
| | | | ENST00000585517 | | | | |
| Genewiz | TFORF2657 | STAT3 | NM_003150, XM_017024975, XM_005257616, | 2747 | 6297 | 9847 | None |
| | | | ENST00000404395 | | | | |
| Genewiz | TFORF2658 | STAT2 | NM_198332, ENST00000557235 | 2748 | 6298 | 9848 | None |
| Genewiz | TFORF2659 | STAT2 | NM_005419, ENST00000314128 | 2749 | 6299 | 9849 | None |
| Genewiz | TFORF2660 | STAT1 | NM_007315, XM_006712718, ENST00000361099, | 2750 | 6300 | 9850 | None |
| | | | ENST00000409465 | | | | |
| Genewiz | TFORF2661 | STOX1 | NM_152709, NM_001130161, ENST00000298596, | 2751 | 6301 | 9851 | None |
| | | | ENST00000399169 | | | | |
| Genewiz | TFORF2662 | STOX1 | NM_001130159, ENST00000399165 | 2752 | 6302 | 9852 | None |
| Genewiz | TFORF2663 | STOX1 | NM_001130160, ENST00000399162 | 2753 | 6303 | 9853 | None |
| Genewiz | TFORF2664 | ZBED1 | NM_001171135, NM_001171136, NM_004729, | 2754 | 6304 | 9854 | None |
| | | | NM_001171135, NM_001171136, NM_004729, | | | | |
| | | | ENST00000381222, ENST00000381223, ENST00000381218, | | | | |
| | | | ENST00000381222, ENST00000381223, ENST00000381218 | | | | |
| Genewiz | TFORF2665 | ZBED2 | NM_024508, ENST00000317012 | 2755 | 6305 | 9855 | None |
| Genewiz | TFORF2666 | ZBED3 | NM_032367, ENST00000255198 | 2756 | 6306 | 9856 | None |
| Genewiz | TFORF2667 | ZBED4 | NM_014838, ENST00000216268 | 2757 | 6307 | 9857 | None |
| Genewiz | TFORF2668 | ZBED6 | NM_001174108, ENST00000550078 | 2758 | 6308 | 9858 | None |
| Genewiz | TFORF2669 | BTAF1 | NM_003972, ENST00000265990 | 2759 | 6309 | 9859 | None |
| Genewiz | TFORF2670 | NEUROD2 | NM_006160, ENST00000302584 | 2760 | 6310 | 9860 | None |
| Genewiz | TFORF2671 | NEUROD4 | NM_021191, ENST00000242994 | 2761 | 6311 | 9861 | None |
| Genewiz | TFORF2672 | CHURC1 | NM_145165, ENST00000607599 | 2762 | 6312 | 9862 | None |
| Genewiz | TFORF2673 | CHURC1 | NM_001204063, ENST00000549115 | 2763 | 6313 | 9863 | None |
| Genewiz | TFORF2674 | CHURC1 | NM_001204064, ENST00000548752 | 2764 | 6314 | 9864 | None |
| Genewiz | TFORF2675 | CCDC169- | NM_001198910, ENST00000511166 | 2765 | 6315 | 9865 | None |
| | | SOHLH2 | | | | | |
| Genewiz | TFORF2676 | ZNF705A | NM_001004328, ENST00000610508, ENST00000359286 | 2766 | 6316 | 9866 | None |
| Genewiz | TFORF2677 | GABPA | NM_001197297, NM_002040, XM_011529520, | 2767 | 6317 | 9867 | None |
| | | | XM_011529521, XM_005260938, XM_017028313, | | | | |
| | | | ENST00000354828, ENST00000400075 | | | | |
| Genewiz | TFORF2678 | PEG3 | NM_001146185, ENST00000593695 | 2768 | 6318 | 9868 | None |
| Genewiz | TFORF2679 | PEG3 | NM_001146186, NM_006210, NM_001146184, | 2769 | 6319 | 9869 | None |
| | | | ENST00000326441 | | | | |
| Genewiz | TFORF2680 | PEG3 | NM_001146187, ENST00000598410 | 2770 | 6320 | 9870 | None |
| Genewiz | TFORF2681 | ZMYND11 | NM_001202466, ENST00000309776 | 2771 | 6321 | 9871 | None |
| Genewiz | TFORF2682 | ZMYND11 | NM_001202468, ENST00000558098 | 2772 | 6322 | 9872 | None |
| Genewiz | TFORF2683 | ZMYND11 | NM_001202465, ENST00000397959, ENST00000602682 | 2773 | 6323 | 9873 | None |
| Genewiz | TFORF2684 | ZMYND11 | NM_006624, XM_005252359, XM_017015588, | 2774 | 6324 | 9874 | None |
| | | | XM_017015589, XM_017015587, XM_017015590, | | | | |
| | | | ENST00000397962, ENST00000381591 | | | | |
| Genewiz | TFORF2685 | ZMYND11 | XM_017015591, ENST00000381584, ENST00000627286 | 2775 | 6325 | 9875 | None |
| Genewiz | TFORF2686 | ZMYND11 | NM_212479, ENST00000509513 | 2776 | 6326 | 9876 | None |
| Genewiz | TFORF2687 | ZMYND11 | XM_006717376, XM_005252362, XM_017015592, | 2777 | 6327 | 9877 | None |
| | | | XM_005252361, NM_001202464, XM_017015593, | | | | |
| | | | ENST00000381607 | | | | |
| Genewiz | TFORF2688 | TEAD3 | NM_003214, ENST00000338863, ENST00000639578 | 2778 | 6328 | 9878 | None |
| Genewiz | TFORF2689 | TEAD2 | NM_001256661, NM_001256660, XM_006723424, | 2779 | 6329 | 9879 | None |
| | | | ENST00000598810, ENST00000593945 | | | | |
| Genewiz | TFORF2690 | TEAD2 | NM_001256658, NM_001256659, XM_011527403, | 2780 | 6330 | 9880 | None |
| | | | ENST00000601519, ENST00000377214 | | | | |
| Genewiz | TFORF2691 | TEAD2 | NM_001256662, ENST00000539846 | 2781 | 6331 | 9881 | None |
| Genewiz | TFORF2692 | TEAD1 | NM_021961, ENST00000527636, ENST00000638666 | 2782 | 6332 | 9882 | None |
| Genewiz | TFORF2693 | TEAD4 | NM_201443, ENST00000397122 | 2783 | 6333 | 9883 | None |
| Genewiz | TFORF2694 | ELF5 | NM_001243081, ENST00000620316 | 2784 | 6334 | 9884 | None |
| Genewiz | TFORF2695 | ELF5 | NM_001243080, ENST00000429939 | 2785 | 6335 | 9885 | None |
| Genewiz | TFORF2696 | ELF5 | NM_198381, ENST00000312319 | 2786 | 6336 | 9886 | None |
| Genewiz | TFORF2697 | ZFP42 | XM_011531607, XM_011531604, NM_001304358, | 2787 | 6337 | 9887 | None |
| | | | NM_174900, XM_011531605, XM_011531606, | | | | |
| | | | ENST00000509524, ENST00000326866, ENST00000618147 | | | | |
| Genewiz | TFORF2698 | ACTL6A | NM_177989, NM_178042, ENST00000450518, | 2788 | 6338 | 9888 | None |
| | | | ENST00000392662 | | | | |
| Genewiz | TFORF2699 | KMT2B | NM_014727, ENST00000420124 | 2789 | 6339 | 9889 | None |
| Genewiz | TFORF2700 | ESRRG | NM_001438, ENST00000408911 | 2790 | 6340 | 9890 | None |
| Genewiz | TFORF2701 | ESRRG | NM_001243518, XM_011509269, XM_011509267, | 2791 | 6341 | 9891 | None |
| | | | XM_011509268, ENST00000366937 | | | | |
| Genewiz | TFORF2702 | ESRRG | NM_001243507, ENST00000463665 | 2792 | 6342 | 9892 | None |
| Genewiz | TFORF2703 | ESRRA | NM_004451, NM_001282450, ENST00000000442, | 2793 | 6343 | 9893 | None |
| | | | ENST00000405666 | | | | |
| Genewiz | TFORF2704 | ESRRA | NM_001282451, ENST00000406310 | 2794 | 6344 | 9894 | None |
| Genewiz | TFORF2705 | ESRRB | NM_004452, XM_017021086, ENST00000380887, | 2795 | 6345 | 9895 | None |
| | | | ENST00000509242 | | | | |
| Genewiz | TFORF2706 | TFCP2L1 | NM_014553, ENST00000263707 | 2796 | 6346 | 9896 | None |
| Genewiz | TFORF2707 | ZNF718 | XM_017007980, XM_017007979, NM_001289930, | 2797 | 6347 | 9897 | None |
| | | | NM_001289931, ENST00000609714 | | | | |
| Genewiz | TFORF2708 | ARX | NM_139058, ENST00000379044 | 2798 | 6348 | 9898 | None |
| Genewiz | TFORF2709 | ZNF716 | NM_001159279, ENST00000420713 | 2799 | 6349 | 9899 | None |
| Genewiz | TFORF2710 | DMRTA1 | NM_022160, ENST00000325870 | 2800 | 6350 | 9900 | None |
| Genewiz | TFORF2711 | ZNF710 | NM_198526, ENST00000268154 | 2801 | 6351 | 9901 | None |
| Genewiz | TFORF2712 | ZNF711 | XM_005262187, XM_005262186, XM_017029804, | 2802 | 6352 | 9902 | None |
| | | | XM_011531024, XM_017029805, XM_005262188, | | | | |
| | | | ENST00000360700 | | | | |
| Genewiz | TFORF2713 | ZNF711 | XM_017029808, XM_005262189, XM_017029807, | 2803 | 6353 | 9903 | None |
| | | | XM_011531026, NM_021998, XM_017029809, | | | | |
| | | | XM_017029806, ENST00000373165, ENST00000276123 | | | | |
| Genewiz | TFORF2714 | ZNF860 | NM_001137674, XM_017006299, ENST00000360311 | 2804 | 6354 | 9904 | None |
| Genewiz | TFORF2715 | ZNF713 | NM_182633, ENST00000429591 | 2805 | 6355 | 9905 | None |
| Genewiz | TFORF2716 | SLC2A4RG | NM_020062, ENST00000266077 | 2806 | 6356 | 9906 | None |
| Genewiz | TFORF2717 | TFCP2 | NM_001173453, ENST00000548115 | 2807 | 6357 | 9907 | None |
| Genewiz | TFORF2718 | LRPPRC | NM_133259, ENST00000260665 | 2808 | 6358 | 9908 | None |
| Genewiz | TFORF2719 | ZNF79 | NM_001286697, NM_001286696, NM_001322260, | 2809 | 6359 | 9909 | None |
| | | | XM_006717279, ENST00000543471, ENST00000612342 | | | | |
| Genewiz | TFORF2720 | ZNF79 | NM_007135, ENST00000342483 | 2810 | 6360 | 9910 | None |
| Genewiz | TFORF2721 | ZNF79 | NM_001286698, ENST00000617266 | 2811 | 6361 | 9911 | None |
| Genewiz | TFORF2722 | ZNF70 | NM_021916, ENST00000341976 | 2812 | 6362 | 9912 | None |
| Genewiz | TFORF2723 | ZNF76 | XM_017011256, NM_001292032, ENST00000339411 | 2813 | 6363 | 9913 | None |
| Genewiz | TFORF2724 | ZNF74 | NM_001256524, NM_003426, ENST00000400451, | 2814 | 6364 | 9914 | None |
| | | | ENST00000611540 | | | | |
| Genewiz | TFORF2725 | ZNF74 | NM_001256523, ENST00000403682 | 2815 | 6365 | 9915 | None |
| Genewiz | TFORF2726 | ZFP1 | NM_001318476, NM_001318475, ENST00000464850, | 2816 | 6366 | 9916 | None |
| | | | ENST00000567481 | | | | |
| Genewiz | TFORF2727 | ZFP1 | NM_001318472, NM_001318471, XM_017022986, | 2817 | 6367 | 9917 | None |
| | | | ENST00000332307 | | | | |
| Genewiz | TFORF2728 | ZFP1 | NM_153688, NM_001318469, XM_017022984, | 2818 | 6368 | 9918 | None |
| | | | XM_017022985, ENST00000570010, ENST00000393430 | | | | |
| Genewiz | TFORF2729 | ZNF668 | NM_001172669, ENST00000426488, ENST00000539836 | 2819 | 6369 | 9919 | None |
| Genewiz | TFORF2730 | ZNF668 | NM_001172670, NM_024706, NM_001172668, | 2820 | 6370 | 9920 | None |
| | | | ENST00000535577, ENST00000538906, ENST00000394983, | | | | |
| | | | ENST00000300849 | | | | |
| Genewiz | TFORF2731 | ZNF669 | NM_024804, ENST00000343381 | 2821 | 6371 | 9921 | None |
| Genewiz | TFORF2732 | ZNF669 | NM_001142572, ENST00000448299 | 2822 | 6372 | 9922 | None |
| Genewiz | TFORF2733 | BDP1 | NM_018429, ENST00000358731 | 2823 | 6373 | 9923 | None |
| Genewiz | TFORF2734 | ZNF662 | NM_207404, ENST00000440367 | 2824 | 6374 | 9924 | None |
| Genewiz | TFORF2735 | ZNF662 | NM_001134656, ENST00000328199 | 2825 | 6375 | 9925 | None |
| Genewiz | TFORF2736 | PLK4 | NM_001190801, ENST00000514379 | 2826 | 6376 | 9926 | None |
| Genewiz | TFORF2737 | PLK4 | NM_014264, ENST00000270861 | 2827 | 6377 | 9927 | None |
| Genewiz | TFORF2738 | PLK4 | NM_001190799, ENST00000513090 | 2828 | 6378 | 9928 | None |
| Genewiz | TFORF2739 | ZNF665 | NM_024733, XM_005259266, ENST00000396424 | 2829 | 6379 | 9929 | None |
| Genewiz | TFORF2740 | ZNF667 | XM_011527208, XM_011527209, NM_001321356, | 2830 | 6380 | 9930 | None |
| | | | NM_022103, ENST00000504904, ENST00000292069 | | | | |
| Genewiz | TFORF2741 | ZNF484 | XM_017015181, NM_001261459, NM_001261460, | 2831 | 6381 | 9931 | None |
| | | | NM_001007101, ENST00000395506, ENST00000332591 | | | | |
| Genewiz | TFORF2742 | ZNF484 | NM_001261458, ENST00000395505 | 2832 | 6382 | 9932 | None |
| Genewiz | TFORF2743 | T | NM_001270484, ENST00000366871 | 2833 | 6383 | 9933 | None |
| Genewiz | TFORF2744 | T | NM_003181, ENST00000296946 | 2834 | 6384 | 9934 | None |
| Genewiz | TFORF2745 | HELZ2 | NM_033405, ENST00000427522 | 2835 | 6385 | 9935 | None |
| Genewiz | TFORF2746 | HELZ2 | NM_001037335, ENST00000467148 | 2836 | 6386 | 9936 | None |
| Genewiz | TFORF2747 | NKX6-2 | XM_017016789, NM_177400, ENST00000368592 | 2837 | 6387 | 9937 | None |
| Genewiz | TFORF2748 | NKX6-3 | XM_017013143, ENST00000518699 | 2838 | 6388 | 9938 | None |
| Genewiz | TFORF2749 | NKX6-3 | NM_152568, ENST00000524115 | 2839 | 6389 | 9939 | None |
| Genewiz | TFORF2750 | NKX6-1 | NM_006168, ENST00000295886 | 2840 | 6390 | 9940 | None |
| Genewiz | TFORF2751 | ZNF444 | XM_005259035, NM_018337, XM_005259034, | 2841 | 6391 | 9941 | None |
| | | | XM_005259036, XM_011527068, ENST00000337080 | | | | |
| Genewiz | TFORF2752 | ZNF444 | NM_001253792, ENST00000592949 | 2842 | 6392 | 9942 | None |
| Genewiz | TFORF2753 | ZNF445 | NM_181489, XM_011533674, XM_005265102, | 2843 | 6393 | 9943 | None |
| | | | ENST00000396077, ENST00000425708 | | | | |
| Genewiz | TFORF2754 | ZNF446 | XM_005259052, NM_017908, ENST00000594369 | 2844 | 6394 | 9944 | None |
| Genewiz | TFORF2755 | ZNF440 | NM_152357, ENST00000304060 | 2845 | 6395 | 9945 | None |
| Genewiz | TFORF2756 | BARHL2 | NM_020063, ENST00000370445 | 2846 | 6396 | 9946 | None |
| Genewiz | TFORF2757 | BARHL1 | NM_020064, ENST00000263610, ENST00000542090 | 2847 | 6397 | 9947 | None |
| Genewiz | TFORF2758 | ZNF443 | NM_005815, ENST00000301547 | 2848 | 6398 | 9948 | None |
| Genewiz | TFORF2759 | L3MBTL1 | NM_015478, ENST00000373135 | 2849 | 6399 | 9949 | None |
| Genewiz | TFORF2760 | L3MBTL1 | NM_032107, ENST00000427442, ENST00000418998 | 2850 | 6400 | 9950 | None |
| Genewiz | TFORF2761 | ZKSCAN3 | XM_006715215, NM_024493, NM_001242894, | 2851 | 6401 | 9951 | None |
| | | | ENST00000252211, ENST00000377255 | | | | |
| Genewiz | TFORF2762 | ZKSCAN3 | NM_001242895, XM_006715218, ENST00000341464 | 2852 | 6402 | 9952 | None |
| Genewiz | TFORF2763 | ZKSCAN2 | NM_001012981, ENST00000328086 | 2853 | 6403 | 9953 | None |
| Genewiz | TFORF2764 | ZKSCAN5 | XM_017011918, XM_017011919, NM_145102, | 2854 | 6404 | 9954 | None |
| | | | NM_001318082, NM_014569, ENST00000326775, | | | | |
| | | | ENST00000451158, ENST00000394170 | | | | |
| Genewiz | TFORF2765 | ZKSCAN7 | NM_001288590, NM_018651, ENST00000426540, | 2855 | 6405 | 9955 | None |
| | | | ENST00000273320 | | | | |
| Genewiz | TFORF2766 | ZKSCAN7 | NM_001288592, ENST00000447279 | 2856 | 6406 | 9956 | None |
| Genewiz | TFORF2767 | ZKSCAN7 | NM_001288591, NM_025169, ENST00000431636, | 2857 | 6407 | 9957 | None |
| | | | ENST00000341840 | | | | |
| Genewiz | TFORF2768 | KDM5D | NM_001146705, ENST00000541639 | 2858 | 6408 | 9958 | None |
| Genewiz | TFORF2769 | KDM5D | NM_001146706, ENST00000382806 | 2859 | 6409 | 9959 | None |
| Genewiz | TFORF2770 | KDM5D | NM_004653, ENST00000317961 | 2860 | 6410 | 9960 | None |
| Genewiz | TFORF2771 | KDM5A | NM_001042603, ENST00000399788 | 2861 | 6411 | 9961 | None |
| Genewiz | TFORF2772 | KDM5B | NM_001314042, ENST00000367264 | 2862 | 6412 | 9962 | None |
| Genewiz | TFORF2773 | KDM5B | NM_006618, ENST00000367265 | 2863 | 6413 | 9963 | None |
| Genewiz | TFORF2774 | KDMSC | NM_001146702, ENST00000452825 | 2864 | 6414 | 9964 | None |
| Genewiz | TFORF2775 | KDM5C | NM_004187, ENST00000375401 | 2865 | 6415 | 9965 | None |
| Genewiz | TFORF2776 | KDM5C | NM_001282622, ENST00000404049 | 2866 | 6416 | 9966 | None |
| Genewiz | TFORF2777 | KDM5C | XM_011530826, ENST00000375383 | 2867 | 6417 | 9967 | None |
| Genewiz | TFORF2778 | KDMSC | XM_005262035, ENST00000375379 | 2868 | 6418 | 9968 | None |
| Genewiz | TFORF2779 | BATE | NM_006399, ENST00000286639 | 2869 | 6419 | 9969 | None |
| Genewiz | TFORF2780 | ZNF30 | XM_017027426, NM_194325, XM_017027425, | 2870 | 6420 | 9970 | None |
| | | | ENST00000601142 | | | | |
| Genewiz | TFORF2781 | ZNF30 | XM_017027424, NM_001099437, NM_001099438, | 2871 | 6421 | 9971 | None |
| | | | XM_011527443, XM_011527444, XM_017027423, | | | | |
| | | | ENST00000303586, ENST00000439785 | | | | |
| Genewiz | TFORF2782 | GLIS2 | NM_001318918, XM_005255641, NM_032575, | 2872 | 6422 | 9972 | None |
| | | | ENST00000262366, ENST00000433375 | | | | |
| Genewiz | TFORF2783 | GLIS3 | XM_005251386, XM_017014361, XM_011517766, | 2873 | 6423 | 9973 | None |
| | | | NM_152629, ENST00000324333 | | | | |
| Genewiz | TFORF2784 | GLIS3 | XM_011517764, XM_011517763, NM_001042413, | 2874 | 6424 | 9974 | None |
| | | | ENST00000381971 | | | | |
| Genewiz | TFORF2785 | GLIS1 | NM_147193, ENST00000312233 | 2875 | 6425 | 9975 | None |
| Genewiz | TFORF2786 | GLIS1 | XM_017000410, XM_017000408, ENST00000628545 | 2876 | 6426 | 9976 | None |
| Genewiz | TFORF2787 | DCP1A | NM_001290204, ENST00000294241 | 2877 | 6427 | 9977 | None |
| Genewiz | TFORF2788 | OTX2 | NM_172337, NM_001270524, NM_001270523, | 2878 | 6428 | 9978 | None |
| | | | ENST00000408990, ENST00000555006 | | | | |
| Genewiz | TFORF2789 | ZNF287 | XM_011523968, XM_017024887, NM_020653, | 2879 | 6429 | 9979 | None |
| | | | ENST00000395824, ENST00000395825 | | | | |
| Genewiz | TFORF2790 | ZNF284 | XM_011526908, XM_011526907, NM_001037813, | 2880 | 6430 | 9980 | None |
| | | | ENST00000421176 | | | | |
| Genewiz | TFORF2791 | ZNF285 | NM_001291488, ENST00000591679 | 2881 | 6431 | 9981 | None |
| Genewiz | TFORF2792 | ZNF282 | NM_001303481, ENST00000479907 | 2882 | 6432 | 9982 | None |
| Genewiz | TFORF2793 | ZNF282 | NM_003575, ENST00000610704 | 2883 | 6433 | 9983 | None |
| Genewiz | TFORF2794 | ZNF283 | XM_017026630, XM_017026628, XM_017026629, | 2884 | 6434 | 9984 | None |
| | | | NM_181845, XM_017026631, ENST00000618787, | | | | |
| | | | ENST00000324461 | | | | |
| Genewiz | TFORF2795 | ZNF283 | NM_001297752, XM_017026638, XM_017026637, | 2885 | 6435 | 9985 | None |
| | | | XM_017026639, ENST00000588797 | | | | |
| Genewiz | TFORF2796 | ZNF281 | NM_001281294, ENST00000367352 | 2886 | 6436 | 9986 | None |
| Genewiz | TFORF2797 | TAF7 | NM_005642, ENST00000313368 | 2887 | 6437 | 9987 | None |
| Genewiz | TFORF2798 | TAF6 | NM_001190415, ENST00000437822 | 2888 | 6438 | 9988 | None |
| Genewiz | TFORF2799 | TAF6 | NM_005641, NM_139315, XM_006716100, | 2889 | 6439 | 9989 | None |
| | | | ENST00000453269, ENST0452041, ENST00000344095 | | | | |
| Genewiz | TFORF2800 | TAF5 | NM_006951, ENST00000369839 | 2890 | 6440 | 9990 | None |
| Genewiz | TFORF2801 | ETV2 | XM_005258652, ENST00000379026 | 2891 | 6441 | 9991 | None |
| Genewiz | TFORF2802 | ETV2 | NM_001304549, ENST00000379023 | 2892 | 6442 | 9992 | None |
| Genewiz | TFORF2803 | ETV2 | XM_011526624, NM_001300974, ENST00000479824 | 2893 | 6443 | 9993 | None |
| Genewiz | TFORF2804 | ETV2 | NM_014209, ENST00000402764, ENST00000403402 | 2894 | 6444 | 9994 | None |
| Genewiz | TFORF2805 | TAF2 | NM_003184, ENST00000378164 | 2895 | 6445 | 9995 | None |
| Genewiz | TFORF2806 | ETV7 | NM_001207037, ENST00000615781 | 2896 | 6446 | 9996 | None |
| Genewiz | TFORF2807 | ETV7 | NM_001207038, ENST00000373737 | 2897 | 6447 | 9997 | None |
| Genewiz | TFORF2808 | ETV7 | NM_001207041, ENST00000538992 | 2898 | 6448 | 9998 | None |
| Genewiz | TFORF2809 | ETV7 | NM_001207035, ENST00000339796 | 2899 | 6449 | 9999 | None |
| Genewiz | TFORF2810 | ETV7 | NM_001207036, ENST00000373738 | 2900 | 6450 | 10000 | None |
| Genewiz | TFORF2811 | ETV7 | NM_001207040, ENST00000620358 | 2901 | 6451 | 10001 | None |
| Genewiz | TFORF2812 | ETV7 | NM_001207039, ENST00000627426 | 2902 | 6452 | 10002 | None |
| Genewiz | TFORF2813 | FOXD4L1 | NM_012184, ENST00000306507 | 2903 | 6453 | 10003 | None |
| Genewiz | TFORF2814 | FOSB | XM_005258691, ENST00000592436 | 2904 | 6454 | 10004 | None |
| Genewiz | TFORF2815 | FOSB | NM_006732, ENST00000353609 | 2905 | 6455 | 10005 | None |
| Genewiz | TFORF2816 | FOSB | NM_001114171, ENST00000417353 | 2906 | 6456 | 10006 | None |
| Genewiz | TFORF2817 | FOXD4L3 | NM_199135, ENST00000342833 | 2907 | 6457 | 10007 | None |
| Genewiz | TFORF2818 | GON4L | NM_001282861, NM_032292, ENST00000361040 | 2908 | 6458 | 10008 | None |
| Genewiz | TFORF2819 | GON4L | NM_001282858, XM_006711393, XM_006711394, | 2909 | 6459 | 10009 | None |
| | | | ENST00000437809, ENST00000271883 | | | | |
| Genewiz | TFORF2820 | GON4L | NM_001282860, NM_001282856, ENST00000368331, | 2910 | 6460 | 10010 | None |
| | | | ENST00000615926 | | | | |
| Genewiz | TFORF2821 | IKZF5 | NM_001271840, XM_017016550, XM_006717947, | 2911 | 6461 | 10011 | None |
| | | | XM_017016551, XM_017016552, ENST00000368886, | | | | |
| | | | ENST00000617859 | | | | |
| Genewiz | TFORF2822 | IKZF4 | XM_017019809, XM_017019805, XM_017019807, | 2912 | 6462 | 10012 | None |
| | | | XM_017019806, XM_{——}017019810, XM_017019811, | | | | |
| | | | XM_017019808, NM_022465, ENST0262032, | | | | |
| | | | ENST00000431367, ENST00000547167 | | | | |
| Genewiz | TFORF2823 | IKZF1 | XM_011515068, NM_006060, XM_011515069, | 2913 | 6463 | 10013 | None |
| | | | ENST00000331340 | | | | |
| Genewiz | TFORF2824 | IKZF1 | XM_011515076, XM_017011668, XM_011515075, | 2914 | 6464 | 10014 | None |
| | | | NM_001291838, ENST00000343574, ENST00000438033 | | | | |
| Genewiz | TFORF2825 | IKZF1 | NM_001291845, ENST00000413698 | 2915 | 6465 | 10015 | None |
| Genewiz | TFORF2826 | IKZF1 | NM_001220771, ENST00000349824 | 2916 | 6466 | 10016 | None |
| Genewiz | TFORF2827 | IKZF1 | NM_001220768, ENST00000357364 | 2917 | 6467 | 10017 | None |
| Genewiz | TFORF2828 | IKZF3 | NM_001257413, ENST00000377952 | 2918 | 6468 | 10018 | None |
| Genewiz | TFORF2829 | IKZF3 | NM_183230, ENST00000350532 | 2919 | 6469 | 10019 | None |
| Genewiz | TFORF2830 | IKZF3 | NM_183231, ENST00000439016 | 2920 | 6470 | 10020 | None |
| Genewiz | TFORF2831 | IKZF3 | NM_183229, ENST00000351680 | 2921 | 6471 | 10021 | None |
| Genewiz | TFORF2832 | IKZF3 | NM_001257408, ENST00000535189 | 2922 | 6472 | 10022 | None |
| Genewiz | TFORF2833 | IKZF3 | NM_001257410, ENST00000377958 | 2923 | 6473 | 10023 | None |
| Genewiz | TFORF2834 | IKZF3 | NM_001257412, ENST00000394189 | 2924 | 6474 | 10024 | None |
| Genewiz | TFORF2835 | IKZF3 | NM_001257414, ENST00000377945 | 2925 | 6475 | 10025 | None |
| Genewiz | TFORF2836 | IKZF3 | NM_001257411, ENST00000377944 | 2926 | 6476 | 10026 | None |
| Genewiz | TFORF2837 | IKZF3 | NM_183232, ENST00000346243 | 2927 | 6477 | 10027 | None |
| Genewiz | TFORF2838 | IKZF3 | NM_001284514, NM_001284515, NM_001284516, | 2928 | 6478 | 10028 | None |
| | | | ENST00000623724, ENST00000583368 | | | | |
| Genewiz | TFORF2839 | IKZF3 | NM_183228, ENST00000467757 | 2929 | 6479 | 10029 | None |
| Genewiz | TFORF2840 | IKZF3 | NM_001257409, ENST00000439167 | 2930 | 6480 | 10030 | None |
| Genewiz | TFORF2841 | IKZF2 | NM_016260, XM_005246385, XM_005246384, | 2931 | 6481 | 10031 | None |
| | | | XM_011510814, ENST00000434687 | | | | |
| Genewiz | TFORF2842 | IKZF2 | NM_001079526, XM_017003589, XM_017003590, | 2932 | 6482 | 10032 | None |
| | | | ENST00000374319 | | | | |
| Genewiz | TFORF2843 | RHOXF2B | NM_001099685, ENST00000371402 | 2933 | 6483 | 10033 | None |
| Genewiz | TFORF2844 | HNRNPK | NM_001318188, NM_031262, XM_017014668, | 2934 | 6484 | 10034 | None |
| | | | ENST00000351839, ENST00000360384 | | | | |
| Genewiz | TFORF2845 | TCF24 | NM_001193502, XM_017012940, ENST00000563496 | 2935 | 6485 | 10035 | None |
| Genewiz | TFORF2846 | TCF20 | NM_181492, XM_011530354, ENST00000335626 | 2936 | 6486 | 10036 | None |
| Genewiz | TFORF2847 | TCF20 | NM_005650, XM_006724313, XM_005261722, | 2937 | 6487 | 10037 | None |
| | | | ENST00000359486 | | | | |
| Genewiz | TFORF2848 | TCF21 | NM_198392, NM_003206, ENST00000367882, | 2938 | 6488 | 10038 | None |
| | | | ENST00000237316 | | | | |
| Genewiz | TFORF2849 | HMGA2 | NM_003484, ENST00000354636 | 2939 | 6489 | 10039 | None |
| Genewiz | TFORF2850 | HMGA2 | NM_001300918, ENST00000393577 | 2940 | 6490 | 10040 | None |
| Genewiz | TFORF2851 | HMGA2 | NM_003483, ENST00000403681 | 2941 | 6491 | 10041 | None |
| Genewiz | TFORF2852 | HMGA2 | NM_001300919, ENST00000536545 | 2942 | 6492 | 10042 | None |
| Genewiz | TFORF2853 | HMGA2 | XM_017019989, ENST00000393578 | 2943 | 6493 | 10043 | None |
| Genewiz | TFORF2854 | FEV | NM_017521, ENST00000295727 | 2944 | 6494 | 10044 | None |
| Genewiz | TFORF2855 | MAPK8IP1 | XM_005253226, ENST00000395629 | 2945 | 6495 | 10045 | None |
| Genewiz | TFORF2856 | MAPK8IP1 | NM_005456, ENST00000241014 | 2946 | 6496 | 10046 | None |
| Genewiz | TFORF2857 | ID4 | NM_001546, ENST00000378700 | 2947 | 6497 | 10047 | None |
| Genewiz | TFORF2858 | ID3 | NM_002167, ENST00000374561 | 2948 | 6498 | 10048 | None |
| Genewiz | TFORF2859 | ID1 | NM_181353, ENST00000376105 | 2949 | 6499 | 10049 | None |
| Genewiz | TFORF2860 | ID1 | NM_002165, ENST00000376112 | 2950 | 6500 | 10050 | None |
| Genewiz | TFORF2861 | SREBF2 | NM_004599, ENST00000361204 | 2951 | 6501 | 10051 | None |
| Genewiz | TFORF2862 | SREBF1 | NM_001005291, ENST00000355815 | 2952 | 6502 | 10052 | None |
| Genewiz | TFORF2863 | SREBF1 | NM_004176, ENST00000261646 | 2953 | 6503 | 10053 | None |
| Genewiz | TFORF2864 | WIZ | NM_021241, ENST00000263381 | 2954 | 6504 | 10054 | None |
| Genewiz | TFORF2865 | WIZ | XM_005260009, XM_011528163, XM_005260010, | 2955 | 6505 | 10055 | None |
| | | | ENST00000599910 | | | | |
| Genewiz | TFORF2866 | WIZ | XM_005260012, ENST00000599686 | 2956 | 6506 | 10056 | None |
| Genewiz | TFORF2867 | TADA3 | NM_006354, NM_001278270, ENST00000301964, | 2957 | 6507 | 10057 | None |
| | | | ENST00000440161 | | | | |
| Genewiz | TFORF2868 | TADA3 | NM_133480, ENST00000343450 | 2958 | 6508 | 10058 | None |
| Genewiz | TFORF2869 | ZNF865 | NM_001195605, ENST00000568956 | 2959 | 6509 | 10059 | None |
| Genewiz | TFORF2870 | HMX1 | NM_001306142, ENST00000506970 | 2960 | 6510 | 10060 | None |
| Genewiz | TFORF2871 | HMX1 | NM_018942, ENST00000400677 | 2961 | 6511 | 10061 | None |
| Genewiz | TFORF2872 | HMX3 | NM_001105574, ENST00000357878 | 2962 | 6512 | 10062 | None |
| Genewiz | TFORF2873 | ELF1 | XM_005266277, NM_172373, XM_005266276, | 2963 | 6513 | 10063 | None |
| | | | XM_011534950, XM_017020413, XM_017020412, | | | | |
| | | | XM_017020409, XM_017020411, XM_017020410, | | | | |
| | | | ENST00000239882 | | | | |
| Genewiz | TFORF2874 | ELF1 | NM_001145353, ENST00000625359 | 2964 | 6514 | 10064 | None |
| Genewiz | TFORF2875 | ELF3 | NM_004433, NM_001114309, ENST00000359651, | 2965 | 6515 | 10065 | None |
| | | | ENST00000367284, ENST00000367283 | | | | |
| Genewiz | TFORF2876 | ELF2 | NM_006874, ENST00000358635 | 2966 | 6516 | 10066 | None |
| Genewiz | TFORF2877 | ELF2 | NM_001276458, ENST00000510408 | 2967 | 6517 | 10067 | None |
| Genewiz | TFORF2878 | ELF2 | NM_001276457, ENST00000379549 | 2968 | 6518 | 10068 | None |
| Genewiz | TFORF2879 | ELF2 | NM_201999, XM_005262804, ENST00000394235 | 2969 | 6519 | 10069 | None |
| Genewiz | TFORF2880 | ELF2 | XM_005262803, ENST00000379550 | 2970 | 6520 | 10070 | None |
| Genewiz | TFORF2881 | XBP1 | NM_005080, ENST00000216037 | 2971 | 6521 | 10071 | None |
| Genewiz | TFORF2882 | XBP1 | NM_001079539, ENST00000611155, ENST00000344347 | 2972 | 6522 | 10072 | None |
| Genewiz | TFORF2883 | BHLHA9 | NM_001164405, ENST00000391429 | 2973 | 6523 | 10073 | None |
| Genewiz | TFORF2884 | FIGLA | NM_001004311, ENST00000332372 | 2974 | 6524 | 10074 | None |
| Genewiz | TFORF2885 | GLI2 | NM_005270, ENST00000452319, ENST00000361492 | 2975 | 6525 | 10075 | None |
| Genewiz | TFORF2886 | GLI3 | NM_000168, ENST00000395925 | 2976 | 6526 | 10076 | None |
| Genewiz | TFORF2887 | BRPF1 | NM_001319050, ENST00000424362 | 2977 | 6527 | 10077 | None |
| Genewiz | TFORF2888 | BRPF1 | NM_001319049, ENST00000433861 | 2978 | 6528 | 10078 | None |
| Genewiz | TFORF2889 | BRPF1 | NM_004634, ENST00000457855 | 2979 | 6529 | 10079 | None |
| Genewiz | TFORF2890 | GLI1 | NM_005269, XM_011538189, ENST00000228682 | 2980 | 6530 | 10080 | None |
| Genewiz | TFORF2891 | GLI1 | NM_001160045, ENST00000543426 | 2981 | 6531 | 10081 | None |
| Genewiz | TFORF2892 | GLI1 | NM_001167609, ENST00000546141 | 2982 | 6532 | 10082 | None |
| Genewiz | TFORF2893 | GLI4 | NM_138465, ENST00000340042, ENST00000523522 | 2983 | 6533 | 10083 | None |
| Genewiz | TFORF2894 | MTA1 | NM_001203258, ENST00000438610 | 2984 | 6534 | 10084 | None |
| Genewiz | TFORF2895 | MTA1 | NM_004689, ENST00000331320 | 2985 | 6535 | 10085 | None |
| Genewiz | TFORF2896 | MTA2 | XM_017018561, ENST00000524902, ENST00000527204 | 2986 | 6536 | 10086 | None |
| Genewiz | TFORF2897 | MTA2 | NM_004739, ENST00000278823 | 2987 | 6537 | 10087 | None |
| Genewiz | TFORF2898 | MTA3 | NM_001282755, NM_001282756, ENST00000405592, | 2988 | 6538 | 10088 | None |
| | | | ENST00000406652 | | | | |
| Genewiz | TFORF2899 | MTA3 | NM_020744, ENST00000407270 | 2989 | 6539 | 10089 | None |
| Genewiz | TFORF2900 | MTA3 | XM_005264456, ENST00000405094 | 2990 | 6540 | 10090 | None |
| Genewiz | TFORF2901 | DACH1 | NM_080760, ENST00000611519 | 2991 | 6541 | 10091 | None |
| Genewiz | TFORF2902 | DACH1 | NM_004392, ENST00000620444 | 2992 | 6542 | 10092 | None |
| Genewiz | TFORF2903 | DACH1 | XM_011534939, ENST00000619232 | 2993 | 6543 | 10093 | None |
| Genewiz | TFORF2904 | DACH1 | NM_080759, ENST00000613252 | 2994 | 6544 | 10094 | None |
| Genewiz | TFORF2905 | DACH2 | NM_053281, ENST00000373125 | 2995 | 6545 | 10095 | None |
| Genewiz | TFORF2906 | DACH2 | NM_001139515, ENST00000508860 | 2996 | 6546 | 10096 | None |
| Genewiz | TFORF2907 | DACH2 | NM_001139514, XM_017029256, ENST00000373131 | 2997 | 6547 | 10097 | None |
| Genewiz | TFORF2908 | NFIC | NM_001245002, ENST00000443272 | 2998 | 6548 | 10098 | None |
| Genewiz | TFORF2909 | NFIC | XM_017026835, NM_001245004, ENST00000590282 | 2999 | 6549 | 10099 | None |
| Genewiz | TFORF2910 | NFIC | NM_001245005, ENST00000395111 | 3000 | 6550 | 10100 | None |
| Genewiz | TFORF2911 | NFIC | NM_005597, ENST00000341919 | 3001 | 6551 | 10101 | None |
| Genewiz | TFORF2912 | NFIC | NM_205843, ENST00000589123 | 3002 | 6552 | 10102 | None |
| Genewiz | TFORF2913 | NFIB | XM_005251467, ENST00000397581 | 3003 | 6553 | 10103 | None |
| Genewiz | TFORF2914 | NFIB | NM_001190738, ENST00000380934 | 3004 | 6554 | 10104 | None |
| Genewiz | TFORF2915 | NFIB | XM_017014742, ENST00000636735 | 3005 | 6555 | 10105 | None |
| Genewiz | TFORF2916 | NFIB | NM_001282787, ENST00000543693 | 3006 | 6556 | 10106 | None |
| Genewiz | TFORF2917 | NFIB | XM_005251470, ENST00000397579 | 3007 | 6557 | 10107 | None |
| Genewiz | TFORF2918 | NFIB | NM_001190737, ENST00000380953 | 3008 | 6558 | 10108 | None |
| Genewiz | TFORF2919 | NFIB | XM_017014740, ENST00000606230 | 3009 | 6559 | 10109 | None |
| Genewiz | TFORF2920 | NFIB | XM_005251469, ENST00000397575 | 3010 | 6560 | 10110 | None |
| Genewiz | TFORF2921 | NFIA | NM_001145512, ENST00000371189 | 3011 | 6561 | 10111 | None |
| Genewiz | TFORF2922 | NFIA | NM_001145511, ENST00000407417 | 3012 | 6562 | 10112 | None |
| Genewiz | TFORF2923 | NFIA | NM_001134673, ENST00000403491 | 3013 | 6563 | 10113 | None |
| Genewiz | TFORF2924 | NFIA | NM_005595, ENST00000371187 | 3014 | 6564 | 10114 | None |
| Genewiz | TFORF2925 | ZFP57 | NM_001109809, XM_011514570, ENST00000488757 | 3015 | 6565 | 10115 | None |
| Genewiz | TFORF2926 | BCL6B | NM_181844, ENST00000293805 | 3016 | 6566 | 10116 | None |
| Genewiz | TFORF2927 | NFIX | XM_005259918, ENST00000592199 | 3017 | 6567 | 10117 | None |
| Genewiz | TFORF2928 | NFIX | NM_002501, ENST00000397661 | 3018 | 6568 | 10118 | None |
| Genewiz | TFORF2929 | NFIX | XM_005259920, ENST00000585575 | 3019 | 6569 | 10119 | None |
| Genewiz | TFORF2930 | NFIX | NM_001271044, ENST00000587760 | 3020 | 6570 | 10120 | None |
| Broad GPP | TFORF2931 | LHX4 | NM_033343.3 | 3021 | 6571 | 10121 | V5 |
| Broad GPP | TFORF2932 | TFAP2A | NM_001032280.2 | 3022 | 6572 | 10122 | V5 |
| Broad GPP | TFORF2933 | HOXD3 | NM_006898.4 | 3023 | 6573 | 10123 | V5 |
| Broad GPP | TFORF2934 | TFEB | NM_007162.2 | 3024 | 6574 | 10124 | V5 |
| Broad GPP | TFORF2935 | MAFK | NM_002360.3 | 3025 | 6575 | 10125 | V5 |
| Broad GPP | TFORF2936 | FOS | NM_005252.3 | 3026 | 6576 | 10126 | V5 |
| Broad GPP | TFORF2937 | FOS | NM_005252.3 | 3027 | 6577 | 10127 | None |
| Broad GPP | TFORF2938 | SOX5 | NM_001261414.2 | 3028 | 6578 | 10128 | V5 |
| Broad GPP | TFORF2939 | HOXA6 | NM_024014.3 | 3029 | 6579 | 10129 | V5 |
| Broad GPP | TFORF2940 | NR4A1 | NM_002135.4 | 3030 | 6580 | 10130 | V5 |
| Broad GPP | TFORF2941 | DLX5 | NM_005221.5 | 3031 | 6581 | 10131 | V5 |
| Broad GPP | TFORF2942 | ESRRG | XM_011509277.1 | 3032 | 6582 | 10132 | V5 |
| Broad GPP | TFORF2943 | ESRRG | NM_001243509.1 | 3033 | 6583 | 10133 | None |
| Broad GPP | TFORF2944 | SATB2 | NM_001172509.1 | 3034 | 6584 | 10134 | V5 |
| Genewiz | TFORF2945 | NEUROD1 | NM_002500.4 | 3035 | 6585 | 10135 | None |
| Broad GPP | TFORF2946 | MYOD1 | NM_002478.4 | 3036 | 6586 | 10136 | V5 |
| Broad GPP | TFORF2947 | HOXC8 | NM_022658.3 | 3037 | 6587 | 10137 | V5 |
| Broad GPP | TFORF2948 | HOXB6 | NM_018952.4 | 3038 | 6588 | 10138 | V5 |
| Broad GPP | TFORF2949 | GCM2 | NM_004752.3 | 3039 | 6589 | 10139 | V5 |
| Broad GPP | TFORF2950 | MAFB | NM_005461.4 | 3040 | 6590 | 10140 | V5 |
| Broad GPP | TFORF2951 | TWIST2 | NM_057179.2 | 3041 | 6591 | 10141 | V5 |
| Broad GPP | TFORF2952 | TLE1 | NM_005077.4 | 3042 | 6592 | 10142 | V5 |
| Broad GPP | TFORF2953 | TLE1 | NM_005077.4 | 3043 | 6593 | 10143 | V5 |
| Broad GPP | TFORF2954 | HNF4G | NM_001330561.1 | 3044 | 6594 | 10144 | None |
| Broad GPP | TFORF2955 | HMG20B | NM_006339.2 | 3045 | 6595 | 10145 | V5 |
| Broad GPP | TFORF2956 | HNF4A | NM_178850.2 | 3046 | 6596 | 10146 | V5 |
| Broad GPP | TFORF2957 | HNF4A | NM_000457.4 | 3047 | 6597 | 10147 | None |
| Broad GPP | TFORF2958 | SP1 | NM_138473.2 | 3048 | 6598 | 10148 | None |
| Broad GPP | TFORF2959 | KAT2A | NM_021078.2 | 3049 | 6599 | 10149 | None |
| Broad GPP | TFORF2960 | PGR | NM_000926.4 | 3050 | 6600 | 10150 | None |
| Broad GPP | TFORF2961 | NR2C1 | NM_001032287.2 | 3051 | 6601 | 10151 | V5 |
| Broad GPP | TFORF2962 | NR2C2 | XM_011534061.2 | 3052 | 6602 | 10152 | V5 |
| Broad GPP | TFORF2963 | NR2C2 | NM_003298.4 | 3053 | 6603 | 10153 | None |
| Broad GPP | TFORF2964 | NR2C2 | XM_011534061.2 | 3054 | 6604 | 10154 | None |
| Broad GPP | TFORF2965 | NR2C2 | NM_003298.4 | 3055 | 6605 | 10155 | V5 |
| Broad GPP | TFORF2966 | NROB1 | NM_000475.4 | 3056 | 6606 | 10156 | V5 |
| Broad GPP | TFORF2967 | NROB1 | NM_000475.4 | 3057 | 6607 | 10157 | None |
| Broad GPP | TFORF2968 | NROB1 | NM_000475.4 | 3058 | 6608 | 10158 | V5 |
| Broad GPP | TFORF2969 | PAX8 | NM_003466.3 | 3059 | 6609 | 10159 | V5 |
| Broad GPP | TFORF2970 | RORC | NM_005060.3 | 3060 | 6610 | 10160 | V5 |
| Broad GPP | TFORF2971 | NR1H4 | NM_001206977.1 | 3061 | 6611 | 10161 | V5 |
| Broad GPP | TFORF2972 | NR1H4 | NM_005123.3 | 3062 | 6612 | 10162 | None |
| Broad GPP | TFORF2973 | PPARD | NM_177435.2 | 3063 | 6613 | 10163 | V5 |
| Genewiz | TFORF2974 | NR1H2 | NM_007121.5 | 3064 | 6614 | 10164 | None |
| Genewiz | TFORF2975 | NAT10 | NM_024662.2 | 3065 | 6615 | 10165 | None |
| Broad GPP | TFORF2976 | TULP2 | NM_003323.2 | 3066 | 6616 | 10166 | V5 |
| Broad GPP | TFORF2977 | ZNF225 | NM_013362.3 | 3067 | 6617 | 10167 | V5 |
| Broad GPP | TFORF2978 | NROB2 | NM_021969.2 | 3068 | 6618 | 10168 | V5 |
| Broad GPP | TFORF2979 | NROB2 | NM_021969.2 | 3069 | 6619 | 10169 | None |
| Broad GPP | TFORF2980 | FOXP3 | NM_014009.3 | 3070 | 6620 | 10170 | V5 |
| Broad GPP | TFORF2981 | ZNF71 | NM_021216.4 | 3071 | 6621 | 10171 | V5 |
| Broad GPP | TFORF2982 | ZNF420 | NM_144689.4 | 3072 | 6622 | 10172 | V5 |
| Broad GPP | TFORF2983 | SUZ12 | NM_015355.3 | 3073 | 6623 | 10173 | V5 |
| Broad GPP | TFORF2984 | MAP3K7 | NM_145333.2 | 3074 | 6624 | 10174 | None |
| Broad GPP | TFORF2985 | TEAD2 | NM_003598.1 | 3075 | 6625 | 10175 | V5 |
| Broad GPP | TFORF2986 | MECP2 | NM_004992.3 | 3076 | 6626 | 10176 | V5 |
| Broad GPP | TFORF2987 | RORA | NM_134261.2 | 3077 | 6627 | 10177 | None |
| Broad GPP | TFORF2988 | OTX2 | NM_021728.3 | 3078 | 6628 | 10178 | V5 |
| Genewiz | TFORF2989 | SLC45A2 | NM_001012509.3 | 3079 | 6629 | 10179 | None |
| Broad GPP | TFORF2990 | NFE2L1 | NM_001330262.1 | 3080 | 6630 | 10180 | V5 |
| Broad GPP | TFORF2991 | ID2 | NM_002166.4 | 3081 | 6631 | 10181 | V5 |
| Broad GPP | TFORF2992 | FOXR2 | NM_198451.3 | 3082 | 6632 | 10182 | V5 |
| Broad GPP | TFORF2993 | SKP2 | NM_032637.3 | 3083 | 6633 | 10183 | V5 |
| Broad GPP | TFORF2994 | SKP2 | NM_005983.3 | 3084 | 6634 | 10184 | None |
| Broad GPP | TFORF2995 | SKP2 | NM_032637.3 | 3085 | 6635 | 10185 | None |
| Broad GPP | TFORF2996 | TGIF1 | NM_173208.2 | 3086 | 6636 | 10186 | V5 |
| Broad GPP | TFORF2997 | GATA2 | NM_001145662.1 | 3087 | 6637 | 10187 | V5 |
| Broad GPP | TFORF2998 | GATA2 | NM_032638.4 | 3088 | 6638 | 10188 | V5 |
| Genewiz | TFORF2999 | CDK2 | NM_001798.4 | 3089 | 6639 | 10189 | None |
| Broad GPP | TFORF3000 | E2F6 | NM_198256.3 | 3090 | 6640 | 10190 | V5 |
| Broad GPP | TFORF3001 | DR1 | NM_001938.2 | 3091 | 6641 | 10191 | V5 |
| Broad GPP | TFORF3002 | NFYC | NM_014223.4 | 3092 | 6642 | 10192 | V5 |
| Broad GPP | TFORF3003 | MIER1 | NM_001146113.1 | 3093 | 6643 | 10193 | V5 |
| Broad GPP | TFORF3004 | PCGF2 | NM_007144.2 | 3094 | 6644 | 10194 | V5 |
| Broad GPP | TFORF3005 | SMAD4 | NM_005359.5 | 3095 | 6645 | 10195 | V5 |
| Broad GPP | TFORF3006 | ZBTB12 | NM_181842.2 | 3096 | 6646 | 10196 | V5 |
| Broad GPP | TFORF3007 | SMAD1 | NM_005900.2 | 3097 | 6647 | 10197 | V5 |
| Genewiz | TFORF3008 | CDX2 | NM_001265.4 | 3098 | 6648 | 10198 | None |
| Broad GPP | TFORF3009 | ZNF35 | NM_003420.3 | 3099 | 6649 | 10199 | V5 |
| Broad GPP | TFORF3010 | NR113 | NM_001077480.2 | 3100 | 6650 | 10200 | V5 |
| Broad GPP | TFORF3011 | ZNF10 | NM_015394.4 | 3101 | 6651 | 10201 | V5 |
| Broad GPP | TFORF3012 | HSF2 | NM_001243094.1 | 3102 | 6652 | 10202 | V5 |
| Broad GPP | TFORF3013 | MEF2A | NM_005587.3 | 3103 | 6653 | 10203 | V5 |
| Broad GPP | TFORF3014 | CEBPG | NM_001806.3 | 3104 | 6654 | 10204 | V5 |
| Broad GPP | TFORF3015 | PPARA | NM_001001928.2 | 3105 | 6655 | 10205 | None |
| Broad GPP | TFORF3016 | MLX | NM_198204.1 | 3106 | 6656 | 10206 | V5 |
| Broad GPP | TFORF3017 | ETV4 | NM_001261439.1 | 3107 | 6657 | 10207 | V5 |
| Broad GPP | TFORF3018 | NEUROG3 | NM_020999.3 | 3108 | 6658 | 10208 | V5 |
| Broad GPP | TFORF3019 | POU2F1 | XM_011509654.2 | 3109 | 6659 | 10209 | V5 |
| Broad GPP | TFORF3020 | PAX7 | NM_013945.2 | 3110 | 6660 | 10210 | V5 |
| Broad GPP | TFORF3021 | DLX4 | NM_138281.2 | 3111 | 6661 | 10211 | V5 |
| Broad GPP | TFORF3022 | RXRG | NM_006917.4 | 3112 | 6662 | 10212 | V5 |
| Broad GPP | TFORF3023 | NEUROD6 | NM_022728.3 | 3113 | 6663 | 10213 | None |
| Broad GPP | TFORF3024 | HEY2 | NM_012259.2 | 3114 | 6664 | 10214 | V5 |
| Broad GPP | TFORF3025 | MYF6 | NM_002469.2 | 3115 | 6665 | 10215 | V5 |
| Broad GPP | TFORF3026 | CREB1 | NM_134442.4 | 3116 | 6666 | 10216 | V5 |
| Broad GPP | TFORF3027 | SP6 | NM_199262.2 | 3117 | 6667 | 10217 | V5 |
| Broad GPP | TFORF3028 | ZNF200 | NM_003454.3 | 3118 | 6668 | 10218 | V5 |
| Broad GPP | TFORF3029 | ATOH1 | NM_005172.1 | 3119 | 6669 | 10219 | V5 |
| Broad GPP | TFORF3030 | ZNF415 | NM_001352148.1 | 3120 | 6670 | 10220 | V5 |
| Broad GPP | TFORF3031 | STRAP | NM_007178.3 | 3121 | 6671 | 10221 | V5 |
| Broad GPP | TFORF3032 | SIRT6 | NM_016539.3 | 3122 | 6672 | 10222 | V5 |
| Broad GPP | TFORF3033 | NFKBIA | NM_020529.2 | 3123 | 6673 | 10223 | V5 |
| Broad GPP | TFORF3034 | OTX1 | NM_001199770.1 | 3124 | 6674 | 10224 | V5 |
| Broad GPP | TFORF3035 | NFKBIB | NM_002503.4 | 3125 | 6675 | 10225 | V5 |
| Broad GPP | TFORF3036 | ATF4 | NM_001675.4 | 3126 | 6676 | 10226 | V5 |
| Broad GPP | TFORF3037 | IRF2 | NM_002199.3 | 3127 | 6677 | 10227 | V5 |
| Broad GPP | TFORF3038 | IRF2 | NM_002199.3 | 3128 | 6678 | 10228 | None |
| Broad GPP | TFORF3039 | NR1H3 | NM_001130102.2 | 3129 | 6679 | 10229 | V5 |
| Broad GPP | TFORF3040 | ZIM3 | NM_052882.1 | 3130 | 6680 | 10230 | V5 |
| Broad GPP | TFORF3041 | SOX15 | NM_006942.1 | 3131 | 6681 | 10231 | V5 |
| Broad GPP | TFORF3042 | TAF9 | NM_003187.4 | 3132 | 6682 | 10232 | V5 |
| Broad GPP | TFORF3043 | ZNF75D | NM_007131.4 | 3133 | 6683 | 10233 | V5 |
| Broad GPP | TFORF3044 | ZNF582 | NM_144690.2 | 3134 | 6684 | 10234 | V5 |
| Broad GPP | TFORF3045 | SNW1 | NM_012245.2 | 3135 | 6685 | 10235 | V5 |
| Broad GPP | TFORF3046 | ZNF418 | NM_133460.2 | 3136 | 6686 | 10236 | V5 |
| Broad GPP | TFORF3047 | ZNF266 | NM_006631.3 | 3137 | 6687 | 10237 | V5 |
| Broad GPP | TFORF3048 | ATMIN | NM_001300728.1 | 3138 | 6688 | 10238 | V5 |
| Broad GPP | TFORF3049 | CDK1 | NM_001786.4 | 3139 | 6689 | 10239 | V5 |
| Broad GPP | TFORF3050 | CDK1 | NM_001786.4 | 3140 | 6690 | 10240 | None |
| Broad GPP | TFORF3051 | CDK1 | NM_001786.4 | 3141 | 6691 | 10241 | None |
| Broad GPP | TFORF3052 | NCOA3 | NM_006534.3 | 3142 | 6692 | 10242 | V5 |
| Broad GPP | TFORF3053 | ZBTB44 | NM_014155.4 | 3143 | 6693 | 10243 | V5 |
| Broad GPP | TFORF3054 | SALL4 | NM_020436.4 | 3144 | 6694 | 10244 | V5 |
| Broad GPP | TFORF3055 | THRB | NM_001128177.1 | 3145 | 6695 | 10245 | V5 |
| Broad GPP | TFORF3056 | THRB | NM_001128177.1 | 3146 | 6696 | 10246 | None |
| Broad GPP | TFORF3057 | YAF2 | NM_001190979.2 | 3147 | 6697 | 10247 | V5 |
| Genewiz | TFORF3058 | EBF1 | NM_024007.4 | 3148 | 6698 | 10248 | None |
| Broad GPP | TFORF3059 | FOSL1 | NM_005438.4 | 3149 | 6699 | 10249 | V5 |
| Broad GPP | TFORF3060 | BACH1 | NM_206866.2 | 3150 | 6700 | 10250 | V5 |
| Broad GPP | TFORF3061 | SNAPC3 | NM_001039697.1 | 3151 | 6701 | 10251 | V5 |
| Genewiz | TFORF3062 | ZNF549 | NM_001199295.1 | 3152 | 6702 | 10252 | None |
| Broad GPP | TFORF3063 | FOXP1 | NM_001012505.1 | 3153 | 6703 | 10253 | V5 |
| Broad GPP | TFORF3064 | TERF1 | NM_003218.3 | 3154 | 6704 | 10254 | V5 |
| Broad GPP | TFORF3065 | ZBTB1 | NM_001123329.1 | 3155 | 6705 | 10255 | V5 |
| Broad GPP | TFORF3066 | CXXC1 | NM_001101654.1 | 3156 | 6706 | 10256 | V5 |
| Broad GPP | TFORF3067 | ZNF8 | NM_021089.2 | 3157 | 6707 | 10257 | V5 |
| Broad GPP | TFORF3068 | FOXJ2 | NM_018416.2 | 3158 | 6708 | 10258 | V5 |
| Broad GPP | TFORF3069 | DPF2 | NM_006268.4 | 3159 | 6709 | 10259 | V5 |
| Broad GPP | TFORF3070 | ZNF322 | NM_024639.4 | 3160 | 6710 | 10260 | V5 |
| Broad GPP | TFORF3071 | PRDM5 | NM_001300823.1 | 3161 | 6711 | 10261 | V5 |
| Broad GPP | TFORF3072 | NELFE | NM_002904.5 | 3162 | 6712 | 10262 | V5 |
| Broad GPP | TFORF3073 | LMO2 | NM_001142315.1 | 3163 | 6713 | 10263 | V5 |
| Broad GPP | TFORF3074 | LMO2 | NM_001142315.1 | 3164 | 6714 | 10264 | V5 |
| Broad GPP | TFORF3075 | ETS2 | NM_005239.5 | 3165 | 6715 | 10265 | V5 |
| Broad GPP | TFORF3076 | GTF2A2 | NM_004492.2 | 3166 | 6716 | 10266 | V5 |
| Broad GPP | TFORF3077 | AP2B1 | NM_001030006.1 | 3167 | 6717 | 10267 | V5 |
| Broad GPP | TFORF3078 | XRCC4 | NM_001318012.1 | 3168 | 6718 | 10268 | V5 |
| Broad GPP | TFORF3079 | ZNF846 | XM_017026405.1 | 3169 | 6719 | 10269 | V5 |
| Broad GPP | TFORF3080 | ZNF296 | NM_145288.2 | 3170 | 6720 | 10270 | V5 |
| Broad GPP | TFORF3081 | ELK3 | NM_005230.3 | 3171 | 6721 | 10271 | V5 |
| Broad GPP | TFORF3082 | GBX2 | NM_001485.3 | 3172 | 6722 | 10272 | V5 |
| Broad GPP | TFORF3083 | FOSL2 | NM_005253.3 | 3173 | 6723 | 10273 | V5 |
| Broad GPP | TFORF3084 | ZFP36L1 | NM_001244698.1 | 3174 | 6724 | 10274 | V5 |
| Broad GPP | TFORF3085 | GABPB1 | NM_005254.5 | 3175 | 6725 | 10275 | V5 |
| Broad GPP | TFORF3086 | STAT1 | NM_139266.2 | 3176 | 6726 | 10276 | V5 |
| Broad GPP | TFORF3087 | MAFF | NM_012323.3 | 3177 | 6727 | 10277 | V5 |
| Genewiz | TFORF3088 | E4F1 | NM_001288776.1 | 3178 | 6728 | 10278 | None |
| Broad GPP | TFORF3089 | ZSCAN9 | NM_006299.4 | 3179 | 6729 | 10279 | V5 |
| Broad GPP | TFORF3090 | ZNF260 | NM_001012756.2 | 3180 | 6730 | 10280 | V5 |
| Broad GPP | TFORF3091 | ZNF260 | NM_001012756.2 | 3181 | 6731 | 10281 | V5 |
| Broad GPP | TFORF3092 | NPAS1 | NM_002517.3 | 3182 | 6732 | 10282 | V5 |
| Broad GPP | TFORF3093 | ZNF574 | NM_022752.5 | 3183 | 6733 | 10283 | V5 |
| Broad GPP | TFORF3094 | ZIM2 | NM_015363.4 | 3184 | 6734 | 10284 | V5 |
| Broad GPP | TFORF3095 | LING | NM_173083.3 | 3185 | 6735 | 10285 | V5 |
| Broad GPP | TFORF3096 | ZNF837 | NM_138466.1 | 3186 | 6736 | 10286 | V5 |
| Broad GPP | TFORF3097 | ZNF76 | NM_003427.4 | 3187 | 6737 | 10287 | V5 |
| Broad GPP | TFORF3098 | ZNF467 | NM_207336.2 | 3188 | 6738 | 10288 | V5 |
| Broad GPP | TFORF3099 | KAT7 | NM_007067.4 | 3189 | 6739 | 10289 | V5 |
| Broad GPP | TFORF3100 | ZNF212 | NM_012256.3 | 3190 | 6740 | 10290 | V5 |
| Broad GPP | TFORF3101 | PES1 | NM_014303.3 | 3191 | 6741 | 10291 | V5 |
| Broad GPP | TFORF3102 | ZNF689 | NM_138447.2 | 3192 | 6742 | 10292 | V5 |
| Genewiz | TFORF3103 | HOXA1 | NM_005522.4 | 3193 | 6743 | 10293 | None |
| Broad GPP | TFORF3104 | NKX2-5 | NM_004387.3 | 3194 | 6744 | 10294 | V5 |
| Broad GPP | TFORF3105 | CCNH | NM_001239.3 | 3195 | 6745 | 10295 | V5 |
| Broad GPP | TFORF3106 | CCNH | NM_001239.3 | 3196 | 6746 | 10296 | V5 |
| Broad GPP | TFORF3107 | RARA | NM_001145301.2 | 3197 | 6747 | 10297 | None |
| Broad GPP | TFORF3108 | ASCL2 | NM_005170.2 | 3198 | 6748 | 10298 | V5 |
| Broad GPP | TFORF3109 | ELF5 | NM_001422.3 | 3199 | 6749 | 10299 | V5 |
| Broad GPP | TFORF3110 | TGIF2LY | NM_139214.2 | 3200 | 6750 | 10300 | V5 |
| Broad GPP | TFORF3111 | ZNF416 | NM_017879.1 | 3201 | 6751 | 10301 | V5 |
| Broad GPP | TFORF3112 | RFXANK | NM_003721.3 | 3202 | 6752 | 10302 | V5 |
| Broad GPP | TFORF3113 | RFXANK | NM_001278727.1 | 3203 | 6753 | 10303 | V5 |
| Broad GPP | TFORF3114 | BHLHA15 | NM_177455.3 | 3204 | 6754 | 10304 | V5 |
| Broad GPP | TFORF3115 | GSC | NM_173849.2 | 3205 | 6755 | 10305 | V5 |
| Genewiz | TFORF3116 | HEYL | NM_014571.3 | 3206 | 6756 | 10306 | None |
| Broad GPP | TFORF3117 | MZF1 | NM_003422.2 | 3207 | 6757 | 10307 | V5 |
| Broad GPP | TFORF3118 | ZNF581 | NM_016535.3 | 3208 | 6758 | 10308 | V5 |
| Broad GPP | TFORF3119 | DDIT3 | NM_004083.5 | 3209 | 6759 | 10309 | V5 |
| Broad GPP | TFORF3120 | ZNF341 | NM_001282933.1 | 3210 | 6760 | 10310 | V5 |
| Genewiz | TFORF3121 | ZNF341 | NM_032819.4 | 3211 | 6761 | 10311 | None |
| Broad GPP | TFORF3122 | SATB1 | NM_001322874.1 | 3212 | 6762 | 10312 | V5 |
| Broad GPP | TFORF3123 | STAT4 | NM_001243835.1 | 3213 | 6763 | 10313 | V5 |
| Broad GPP | TFORF3124 | SMAD3 | NM_005902.3 | 3214 | 6764 | 10314 | V5 |
| Broad GPP | TFORF3125 | ZNF300 | NM_052860.2 | 3215 | 6765 | 10315 | V5 |
| Broad GPP | TFORF3126 | IKZF3 | NM_012481.4 | 3216 | 6766 | 10316 | V5 |
| Broad GPP | TFORF3127 | NFIL3 | NM_005384.2 | 3217 | 6767 | 10317 | V5 |
| Broad GPP | TFORF3128 | IRF5 | NM_032643.4 | 3218 | 6768 | 10318 | V5 |
| Broad GPP | TFORF3129 | SMAD7 | NM_005904.3 | 3219 | 6769 | 10319 | V5 |
| Broad GPP | TFORF3130 | IRF8 | NM_002163.2 | 3220 | 6770 | 10320 | V5 |
| Broad GPP | TFORF3131 | NR5A2 | NM_003822.4 | 3221 | 6771 | 10321 | V5 |
| Broad GPP | TFORF3132 | ESR2 | NM_001437.2 | 3222 | 6772 | 10322 | None |
| Broad GPP | TFORF3133 | TCF7 | NM_003202.4 | 3223 | 6773 | 10323 | None |
| Broad GPP | TFORF3134 | TFE3 | NM_006521.5 | 3224 | 6774 | 10324 | None |
| Broad GPP | TFORF3135 | NOTCH2 | NM_024408.3 | 3225 | 6775 | 10325 | None |
| Broad GPP | TFORF3136 | HEY1 | NM_012258.3 | 3226 | 6776 | 10326 | V5 |
| Broad GPP | TFORF3137 | HEY1 | NM_001040708.1 | 3227 | 6777 | 10327 | None |
| Broad GPP | TFORF3138 | PPARG | NM_138712.3 | 3228 | 6778 | 10328 | V5 |
| Broad GPP | TFORF3139 | PPARG | NM_015869.4 | 3229 | 6779 | 10329 | None |
| Broad GPP | TFORF3140 | ZNF718 | NM_001039127.5 | 3230 | 6780 | 10330 | V5 |
| Broad GPP | TFORF3141 | ALX3 | NM_006492.2 | 3231 | 6781 | 10331 | V5 |
| Broad GPP | TFORF3142 | CDX4 | NM_005193.1 | 3232 | 6782 | 10332 | V5 |
| Broad GPP | TFORF3143 | CTBP1 | NM_001012614.1 | 3233 | 6783 | 10333 | V5 |
| Broad GPP | TFORF3144 | MIER2 | NM_001346105.1 | 3234 | 6784 | 10334 | V5 |
| Broad GPP | TFORF3145 | ZNF660 | NM_173658.3 | 3235 | 6785 | 10335 | V5 |
| Broad GPP | TFORF3146 | HSF1 | NM_005526.3 | 3236 | 6786 | 10336 | V5 |
| Broad GPP | TFORF3147 | ZBTB43 | NM_014007.3 | 3237 | 6787 | 10337 | V5 |
| Broad GPP | TFORF3148 | NFE2 | NM_006163.2 | 3238 | 6788 | 10338 | V5 |
| Broad GPP | TFORF3149 | ZNF254 | NM_001278665.1 | 3239 | 6789 | 10339 | V5 |
| Broad GPP | TFORF3150 | ZNF254 | NM_203282.3 | 3240 | 6790 | 10340 | V5 |
| Broad GPP | TFORF3151 | ZNF230 | NM_006300.3 | 3241 | 6791 | 10341 | V5 |
| Broad GPP | TFORF3152 | ZFP3 | NM_153018.2 | 3242 | 6792 | 10342 | V5 |
| Broad GPP | TFORF3153 | ZFP2 | NM_030613.3 | 3243 | 6793 | 10343 | V5 |
| Broad GPP | TFORF3154 | ZFP2 | NM_030613.3 | 3244 | 6794 | 10344 | V5 |
| Broad GPP | TFORF3155 | HLX | NM_021958.3 | 3245 | 6795 | 10345 | V5 |
| Broad GPP | TFORF3156 | ETV5 | NM_004454.2 | 3246 | 6796 | 10346 | V5 |
| Broad GPP | TFORF3157 | SMARCD1 | NM_003076.4 | 3247 | 6797 | 10347 | V5 |
| Broad GPP | TFORF3158 | SMARCB1 | NM_001007468.2 | 3248 | 6798 | 10348 | V5 |
| Broad GPP | TFORF3159 | SMAD2 | NM_005901.5 | 3249 | 6799 | 10349 | V5 |
| Broad GPP | TFORF3160 | SNAI3 | NM_178310.3 | 3250 | 6800 | 10350 | V5 |
| Broad GPP | TFORF3161 | ZNF433 | NM_001308355.1 | 3251 | 6801 | 10351 | V5 |
| Broad GPP | TFORF3162 | ZNF253 | NM_001331134.1 | 3252 | 6802 | 10352 | V5 |
| Broad GPP | TFORF3163 | PPP1R13B | NM_015316.2 | 3253 | 6803 | 10353 | V5 |
| Broad GPP | TFORF3164 | RUVBL1 | NM_003707.2 | 3254 | 6804 | 10354 | V5 |
| Broad GPP | TFORF3165 | TRIM28 | NM_005762.2 | 3255 | 6805 | 10355 | V5 |
| Broad GPP | TFORF3166 | ZNF512B | NM_020713.2 | 3256 | 6806 | 10356 | V5 |
| Broad GPP | TFORF3167 | ZNF512B | NM_020713.2 | 3257 | 6807 | 10357 | V5 |
| Broad GPP | TFORF3168 | FOXM1 | NM_021953.3 | 3258 | 6808 | 10358 | V5 |
| Broad GPP | TFORF3169 | GTF21 | NM_001280800.1 | 3259 | 6809 | 10359 | V5 |
| Broad GPP | TFORF3170 | CNOT3 | NM_014516.3 | 3260 | 6810 | 10360 | V5 |
| Genewiz | TFORF3171 | PSMB4 | NM_002796.2 | 3261 | 6811 | 10361 | None |
| Broad GPP | TFORF3172 | STAT6 | NM_003153.4 | 3262 | 6812 | 10362 | V5 |
| Broad GPP | TFORF3173 | STAT6 | NM_003153.4 | 3263 | 6813 | 10363 | None |
| Broad GPP | TFORF3174 | ARIDSA | NM_001319094.1 | 3264 | 6814 | 10364 | V5 |
| Broad GPP | TFORF3175 | ILF3 | NM_001137673.1 | 3265 | 6815 | 10365 | V5 |
| Broad GPP | TFORF3176 | ZNF544 | NM_014480.3 | 3266 | 6816 | 10366 | V5 |
| Broad GPP | TFORF3177 | NR1D2 | NM_005126.4 | 3267 | 6817 | 10367 | None |
| Broad GPP | TFORF3178 | TFDP2 | NM_001178138.1 | 3268 | 6818 | 10368 | V5 |
| Broad GPP | TFORF3179 | LHX9 | NM_020204.2 | 3269 | 6819 | 10369 | V5 |
| Genewiz | TFORF3180 | RARG | NM_000966.5 | 3270 | 6820 | 10370 | None |
| Broad GPP | TFORF3181 | PATZ1 | NM_032051.1 | 3271 | 6821 | 10371 | V5 |
| Broad GPP | TFORF3182 | ZNF324B | NM_207395.2 | 3272 | 6822 | 10372 | V5 |
| Broad GPP | TFORF3183 | ZNF692 | NM_017865.3 | 3273 | 6823 | 10373 | V5 |
| Broad GPP | TFORF3184 | HSBP1 | NM_001537.3 | 3274 | 6824 | 10374 | V5 |
| Broad GPP | TFORF3185 | TOX2 | NM_032883.2 | 3275 | 6825 | 10375 | V5 |
| Broad GPP | TFORF3186 | GTF2H3 | NM_001516.4 | 3276 | 6826 | 10376 | V5 |
| Broad GPP | TFORF3187 | RAD21 | NM_006265.2 | 3277 | 6827 | 10377 | V5 |
| Broad GPP | TFORF3188 | ZNF394 | NM_032164.3 | 3278 | 6828 | 10378 | V5 |
| Broad GPP | TFORF3189 | ZNF558 | NM_144693.2 | 3279 | 6829 | 10379 | V5 |
| Broad GPP | TFORF3190 | ZNF554 | NM_001102651.1 | 3280 | 6830 | 10380 | V5 |
| Broad GPP | TFORF3191 | ZNF436 | NM_030634.2 | 3281 | 6831 | 10381 | V5 |
| Broad GPP | TFORF3192 | ERG | NM_182918.3 | 3282 | 6832 | 10382 | V5 |
| Broad GPP | TFORF3193 | DND1 | NM_194249.2 | 3283 | 6833 | 10383 | V5 |
| Broad GPP | TFORF3194 | TLX3 | NM_021025.3 | 3284 | 6834 | 10384 | V5 |
| Broad GPP | TFORF3195 | ONECUT1 | NM_004498.2 | 3285 | 6835 | 10385 | V5 |
| Broad GPP | TFORF3196 | ZIC1 | NM_003412.3 | 3286 | 6836 | 10386 | V5 |
| Broad GPP | TFORF3197 | STAT5A | NM_003152.3 | 3287 | 6837 | 10387 | V5 |
| Broad GPP | TFORF3198 | OVOL2 | NM_021220.3 | 3288 | 6838 | 10388 | V5 |
| Broad GPP | TFORF3199 | NFATC1 | NM_172390.2 | 3289 | 6839 | 10389 | V5 |
| Broad GPP | TFORF3200 | RUNX3 | NM_001031680.2 | 3290 | 6840 | 10390 | V5 |
| Broad GPP | TFORF3201 | ZNF829 | NM_001037232.3 | 3291 | 6841 | 10391 | V5 |
| Broad GPP | TFORF3202 | ZNF816 | NM_001202456.1 | 3292 | 6842 | 10392 | V5 |
| Broad GPP | TFORF3203 | ZNF526 | NM_133444.2 | 3293 | 6843 | 10393 | V5 |
| Broad GPP | TFORF3204 | LITAR | NM_004862.3 | 3294 | 6844 | 10394 | V5 |
| Broad GPP | TFORF3205 | FOXO3 | NM_201559.2 | 3295 | 6845 | 10395 | V5 |
| Broad GPP | TFORF3206 | TBR1 | NM_006593.3 | 3296 | 6846 | 10396 | V5 |
| Broad GPP | TFORF3207 | HMGN2 | NM_005517.3 | 3297 | 6847 | 10397 | V5 |
| Broad GPP | TFORF3208 | ZNF317 | NM_020933.4 | 3298 | 6848 | 10398 | V5 |
| Broad GPP | TFORF3209 | ZBTB9 | NM_152735.3 | 3299 | 6849 | 10399 | V5 |
| Broad GPP | TFORF3210 | VSX2 | NM_182894.2 | 3300 | 6850 | 10400 | V5 |
| Broad GPP | TFORF3211 | TADA2B | XM_011513595.1 | 3301 | 6851 | 10401 | V5 |
| Broad GPP | TFORF3212 | ZKSCAN1 | NM_001346581.1 | 3302 | 6852 | 10402 | V5 |
| Broad GPP | TFORF3213 | ZNF572 | NM_152412.2 | 3303 | 6853 | 10403 | V5 |
| Broad GPP | TFORF3214 | ZNF484 | NM_031486.2 | 3304 | 6854 | 10404 | V5 |
| Broad GPP | TFORF3215 | ZNF248 | NM_021045.2 | 3305 | 6855 | 10405 | V5 |
| Broad GPP | TFORF3216 | ATF2 | NM_001256094.1 | 3306 | 6856 | 10406 | V5 |
| Broad GPP | TFORF3217 | ZNF227 | NM_182490.2 | 3307 | 6857 | 10407 | V5 |
| Broad GPP | TFORF3218 | FOXN3 | NM_005197.3 | 3308 | 6858 | 10408 | V5 |
| Broad GPP | TFORF3219 | ZNF677 | NM_001317998.1 | 3309 | 6859 | 10409 | V5 |
| Broad GPP | TFORF3220 | ZNF449 | NM_152695.5 | 3310 | 6860 | 10410 | V5 |
| Broad GPP | TFORF3221 | ZNF770 | NM_014106.3 | 3311 | 6861 | 10411 | V5 |
| Broad GPP | TFORF3222 | FOXS1 | NM_004118.3 | 3312 | 6862 | 10412 | V5 |
| Broad GPP | TFORF3223 | ZMIZ2 | NM_001300959.1 | 3313 | 6863 | 10413 | V5 |
| Genewiz | TFORF3224 | HMGB1 | NM_002128.5 | 3314 | 6864 | 10414 | None |
| Broad GPP | TFORF3225 | ZNF285 | NM_152354.5 | 3315 | 6865 | 10415 | V5 |
| Broad GPP | TFORF3226 | FGF3 | NM_005247.2 | 3316 | 6866 | 10416 | V5 |
| Broad GPP | TFORF3227 | RPA2 | NM_002946.4 | 3317 | 6867 | 10417 | V5 |
| Broad GPP | TFORF3228 | MAX | NM_002382.4 | 3318 | 6868 | 10418 | V5 |
| Broad GPP | TFORF3229 | MAX | NM_145114.2 | 3319 | 6869 | 10419 | V5 |
| Broad GPP | TFORF3230 | FOXJ1 | NM_001454.3 | 3320 | 6870 | 10420 | V5 |
| Broad GPP | TFORF3231 | ZNF423 | NM_015069.4 | 3321 | 6871 | 10421 | V5 |
| Broad GPP | TFORF3232 | JUNB | NM_002229.2 | 3322 | 6872 | 10422 | V5 |
| Broad GPP | TFORF3233 | JUNB | NM_002229.2 | 3323 | 6873 | 10423 | V5 |
| Broad GPP | TFORF3234 | VSX1 | NM_014588.5 | 3324 | 6874 | 10424 | V5 |
| Broad GPP | TFORF3235 | ZNF320 | NM_001351776.1 | 3325 | 6875 | 10425 | V5 |
| Broad GPP | TFORF3236 | E2F8 | NM_024680.3 | 3326 | 6876 | 10426 | V5 |
| Broad GPP | TFORF3237 | SOX10 | NM_006941.3 | 3327 | 6877 | 10427 | V5 |
| Broad GPP | TFORF3238 | GTF2A1 | NM_015859.3 | 3328 | 6878 | 10428 | V5 |
| Broad GPP | TFORF3239 | HOXB5 | NM_002147.3 | 3329 | 6879 | 10429 | V5 |
| Broad GPP | TFORF3240 | POU2AF1 | NM_006235.2 | 3330 | 6880 | 10430 | V5 |
| Broad GPP | TFORF3241 | ZNF490 | NM_020714.2 | 3331 | 6881 | 10431 | V5 |
| Broad GPP | TFORF3242 | ZNF136 | NM_003437.4 | 3332 | 6882 | 10432 | V5 |
| Genewiz | TFORF3243 | ZNF267 | NM_003414.5 | 3333 | 6883 | 10433 | None |
| Broad GPP | TFORF3244 | ZNF483 | XM_017014339.1 | 3334 | 6884 | 10434 | V5 |
| Broad GPP | TFORF3245 | MAFG | NM_002359.3 | 3335 | 6885 | 10435 | V5 |
| Broad GPP | TFORF3246 | HOXC9 | NM_006897.2 | 3336 | 6886 | 10436 | V5 |
| Broad GPP | TFORF3247 | LEF1 | NM_016269.4 | 3337 | 6887 | 10437 | V5 |
| Broad GPP | TFORF3248 | ZNF311 | NM_001010877.3 | 3338 | 6888 | 10438 | V5 |
| Broad GPP | TFORF3249 | ZNF181 | NM_001029997.3 | 3339 | 6889 | 10439 | V5 |
| Broad GPP | TFORF3250 | ZKSCAN8 | NM_006298.3 | 3340 | 6890 | 10440 | V5 |
| Broad GPP | TFORF3251 | PFDN5 | NM_002624.3 | 3341 | 6891 | 10441 | V5 |
| Broad GPP | TFORF3252 | ZNF77 | NM_021217.2 | 3342 | 6892 | 10442 | V5 |
| Broad GPP | TFORF3253 | RBPJ | NM_203283.2 | 3343 | 6893 | 10443 | V5 |
| Genewiz | TFORF3254 | ETV6 | NM_001987.4 | 3344 | 6894 | 10444 | None |
| Broad GPP | TFORF3255 | ZNF175 | NM_007147.3 | 3345 | 6895 | 10445 | V5 |
| Broad GPP | TFORF3256 | ZNF189 | NM_001278231.1 | 3346 | 6896 | 10446 | V5 |
| Broad GPP | TFORF3257 | ZNF800 | NM_176814.4 | 3347 | 6897 | 10447 | V5 |
| Broad GPP | TFORF3258 | TAF12 | NM_005644.3 | 3348 | 6898 | 10448 | V5 |
| Genewiz | TFORF3259 | MEF2D | NM_005920.3 | 3349 | 6899 | 10449 | None |
| Broad GPP | TFORF3260 | ZNF524 | NM_153219.3 | 3350 | 6900 | 10450 | V5 |
| Broad GPP | TFORF3261 | OVOL1 | NM_004561.3 | 3351 | 6901 | 10451 | V5 |
| Broad GPP | TFORF3262 | CEBPE | NM_001805.3 | 3352 | 6902 | 10452 | V5 |
| Broad GPP | TFORF3263 | ZNF26 | NM_019591.3 | 3353 | 6903 | 10453 | V5 |
| Broad GPP | TFORF3264 | ARNTL2 | NM_001248003.1 | 3354 | 6904 | 10454 | V5 |
| Broad GPP | TFORF3265 | ARNTL2 | NM_001248004.1 | 3355 | 6905 | 10455 | V5 |
| Broad GPP | TFORF3266 | ZNF213 | NM_004220.2 | 3356 | 6906 | 10456 | V5 |
| Broad GPP | TFORF3267 | SNAPC2 | NM_003083.3 | 3357 | 6907 | 10457 | V5 |
| Broad GPP | TFORF3268 | ZNF34 | NM_001286769.1 | 3358 | 6908 | 10458 | V5 |
| Broad GPP | TFORF3269 | EZH1 | NM_001991.4 | 3359 | 6909 | 10459 | V5 |
| Broad GPP | TFORF3270 | ZNF138 | NM_001271649.1 | 3360 | 6910 | 10460 | V5 |
| Broad GPP | TFORF3271 | EBF3 | NM_001005463.2 | 3361 | 6911 | 10461 | V5 |
| Broad GPP | TFORF3272 | PRDM4 | NM_012406.3 | 3362 | 6912 | 10462 | V5 |
| Broad GPP | TFORF3273 | BBX | NM_001276286.1 | 3363 | 6913 | 10463 | V5 |
| Broad GPP | TFORF3274 | HMGA1 | NM_145899.2 | 3364 | 6914 | 10464 | V5 |
| Broad GPP | TFORF3275 | HMGA1 | NM_145905.2 | 3365 | 6915 | 10465 | V5 |
| Broad GPP | TFORF3276 | ZBTB14 | NM_001243702.1 | 3366 | 6916 | 10466 | V5 |
| Broad GPP | TFORF3277 | ZNF518A | NM_001278526.1 | 3367 | 6917 | 10467 | V5 |
| Broad GPP | TFORF3278 | MEOX1 | NM_004527.3 | 3368 | 6918 | 10468 | V5 |
| Broad GPP | TFORF3279 | LM01 | NM_002315.2 | 3369 | 6919 | 10469 | V5 |
| Broad GPP | TFORF3280 | DDB2 | NM_000107.2 | 3370 | 6920 | 10470 | V5 |
| Broad GPP | TFORF3281 | ZNF133 | NM_001352456.1 | 3371 | 6921 | 10471 | V5 |
| Broad GPP | TFORF3282 | BRCA1 | NM_007299.3 | 3372 | 6922 | 10472 | V5 |
| Broad GPP | TFORF3283 | HOXA5 | NM_019102.3 | 3373 | 6923 | 10473 | V5 |
| Broad GPP | TFORF3284 | PBX3 | XM_006717133.1 | 3374 | 6924 | 10474 | V5 |
| Broad GPP | TFORF3285 | FEZF1 | NM_001024613.3 | 3375 | 6925 | 10475 | V5 |
| Broad GPP | TFORF3286 | NR2F6 | NM_005234.3 | 3376 | 6926 | 10476 | V5 |
| Broad GPP | TFORF3287 | ZNF114 | NM_001331097.1 | 3377 | 6927 | 10477 | V5 |
| Broad GPP | TFORF3288 | HOXA9 | NM_152739.3 | 3378 | 6928 | 10478 | V5 |
| Genewiz | TFORF3289 | ZFY | NM_001145275.1 | 3379 | 6929 | 10479 | None |
| Broad GPP | TFORF3290 | CTCF | NM_006565.3 | 3380 | 6930 | 10480 | V5 |
| Broad GPP | TFORF3291 | CDK9 | NM_001261.3 | 3381 | 6931 | 10481 | V5 |
| Broad GPP | TFORF3292 | MXD3 | NM_031300.3 | 3382 | 6932 | 10482 | V5 |
| Broad GPP | TFORF3293 | ZNF781 | NM_152605.3 | 3383 | 6933 | 10483 | V5 |
| Broad GPP | TFORF3294 | ZNF784 | NM_203374.1 | 3384 | 6934 | 10484 | V5 |
| Broad GPP | TFORF3295 | SPDEF | NM_012391.2 | 3385 | 6935 | 10485 | V5 |
| Broad GPP | TFORF3296 | HIF1A | NM_001530.3 | 3386 | 6936 | 10486 | None |
| Broad GPP | TFORF3297 | HIF1A | NM_001530.3 | 3387 | 6937 | 10487 | None |
| Broad GPP | TFORF3298 | EZH2 | NM_004456.4 | 3388 | 6938 | 10488 | V5 |
| Broad GPP | TFORF3299 | MEIS2 | NM_001220482.1 | 3389 | 6939 | 10489 | V5 |
| Broad GPP | TFORF3300 | MEIS2 | NM_002399.3 | 3390 | 6940 | 10490 | V5 |
| Broad GPP | TFORF3301 | OLIG3 | NM_175747.2 | 3391 | 6941 | 10491 | V5 |
| Broad GPP | TFORF3302 | SPIC | NM_152323.2 | 3392 | 6942 | 10492 | V5 |
| Broad GPP | TFORF3303 | MITF | NM_198158.2 | 3393 | 6943 | 10493 | V5 |
| Broad GPP | TFORF3304 | MITF | NM_001184968.1 | 3394 | 6944 | 10494 | V5 |
| Broad GPP | TFORF3305 | ESR1 | NM_001122742.1 | 3395 | 6945 | 10495 | V5 |
| Broad GPP | TFORF3306 | THAP1 | NM_018105.2 | 3396 | 6946 | 10496 | V5 |
| Broad GPP | TFORF3307 | ZNF563 | NM_145276.2 | 3397 | 6947 | 10497 | V5 |
| Broad GPP | TFORF3308 | GTF2A1L | NM_006872.4 | 3398 | 6948 | 10498 | V5 |
| Broad GPP | TFORF3309 | DCP1A | NM_018403.6 | 3399 | 6949 | 10499 | V5 |
| Broad GPP | TFORF3310 | ZNF561 | NM_001330365.1 | 3400 | 6950 | 10500 | V5 |
| Broad GPP | TFORF3311 | CREB3 | NM_006368.4 | 3401 | 6951 | 10501 | V5 |
| Broad GPP | TFORF3312 | ELK4 | NM_021795.2 | 3402 | 6952 | 10502 | V5 |
| Broad GPP | TFORF3313 | ZBTB25 | NM_001304507.1 | 3403 | 6953 | 10503 | V5 |
| Broad GPP | TFORF3314 | SMAD9 | NM_005905.5 | 3404 | 6954 | 10504 | V5 |
| Broad GPP | TFORF3315 | ELF4 | NM_001127197.1 | 3405 | 6955 | 10505 | V5 |
| Broad GPP | TFORF3316 | MED21 | NM_004264.4 | 3406 | 6956 | 10506 | V5 |
| Broad GPP | TFORF3317 | ZNF75A | NM_153028.3 | 3407 | 6957 | 10507 | V5 |
| Broad GPP | TFORF3318 | TFCP2 | NM_005653.4 | 3408 | 6958 | 10508 | V5 |
| Broad GPP | TFORF3319 | ZSCAN5A | NM_001322070.1 | 3409 | 6959 | 10509 | V5 |
| Genewiz | TFORF3320 | HDX | NM_001177479.1 | 3410 | 6960 | 10510 | None |
| Broad GPP | TFORF3321 | PAZG4 | NM_006191.2 | 3411 | 6961 | 10511 | V5 |
| Broad GPP | TFORF3322 | ZBTB26 | NM_001304364.1 | 3412 | 6962 | 10512 | V5 |
| Broad GPP | TFORF3323 | ZNF626 | NM_145297.3 | 3413 | 6963 | 10513 | V5 |
| Broad GPP | TFORF3324 | XRCC6 | NM_001288976.1 | 3414 | 6964 | 10514 | V5 |
| Broad GPP | TFORF3325 | DLX3 | NM_005220.2 | 3415 | 6965 | 10515 | V5 |
| Broad GPP | TFORF3326 | ZNF512 | NM_001271287.1 | 3416 | 6966 | 10516 | V5 |
| Broad GPP | TFORF3327 | LHX6 | NM_001242334.1 | 3417 | 6967 | 10517 | V5 |
| Broad GPP | TFORF3328 | ZSCAN16 | NM_001320555.1 | 3418 | 6968 | 10518 | V5 |
| Broad GPP | TFORF3329 | TFAP4 | NM_003223.2 | 3419 | 6969 | 10519 | V5 |
| Broad GPP | TFORF3330 | TBX20 | NM_001166220.1 | 3420 | 6970 | 10520 | V5 |
| Broad GPP | TFORF3331 | PUF60 | NM_014281.4 | 3421 | 6971 | 10521 | V5 |
| Broad GPP | TFORF3332 | PUF60 | NM_001136033.2 | 3422 | 6972 | 10522 | V5 |
| Broad GPP | TFORF3333 | PUF60 | NM_001271100.1 | 3423 | 6973 | 10523 | V5 |
| Broad GPP | TFORF3334 | SP100 | NM_001206702.1 | 3424 | 6974 | 10524 | V5 |
| Broad GPP | TFORF3335 | ZBTB6 | NM_006626.5 | 3425 | 6975 | 10525 | V5 |
| Broad GPP | TFORF3336 | ZBTB37 | NM_032522.4 | 3426 | 6976 | 10526 | V5 |
| Broad GPP | TFORF3337 | HDAC1 | NM_004964.2 | 3427 | 6977 | 10527 | V5 |
| Broad GPP | TFORF3338 | HDAC1 | NM_004964.2 | 3428 | 6978 | 10528 | None |
| Broad GPP | TFORF3339 | HDAC3 | NM_003883.3 | 3429 | 6979 | 10529 | V5 |
| Broad GPP | TFORF3340 | HDAC3 | NM_003883.3 | 3430 | 6980 | 10530 | None |
| Broad GPP | TFORF3341 | HMX2 | NM_005519.1 | 3431 | 6981 | 10531 | V5 |
| Broad GPP | TFORF3342 | TP53BP2 | NM_005426.2 | 3432 | 6982 | 10532 | None |
| Genewiz | TFORF3343 | RARB | NM_000965.4 | 3433 | 6983 | 10533 | None |
| Broad GPP | TFORF3344 | ZNF280A | NM_080740.4 | 3434 | 6984 | 10534 | V5 |
| Broad GPP | TFORF3345 | LMO3 | NM_018640.4 | 3435 | 6985 | 10535 | V5 |
| Broad GPP | TFORF3346 | NHP2 | NM_017838.3 | 3436 | 6986 | 10536 | V5 |
| Broad GPP | TFORF3347 | PITX1 | NM_002653.4 | 3437 | 6987 | 10537 | V5 |
| Broad GPP | TFORF3348 | PITX2 | NM_000325.5 | 3438 | 6988 | 10538 | V5 |
| Broad GPP | TFORF3349 | ERCC8 | NM_001007234.2 | 3439 | 6989 | 10539 | V5 |
| Broad GPP | TFORF3350 | TBX15 | NM_152380.2 | 3440 | 6990 | 10540 | V5 |
| Broad GPP | TFORF3351 | ARNTL | NM_001178.5 | 3441 | 6991 | 10541 | V5 |
| Broad GPP | TFORF3352 | HESX1 | NM_003865.2 | 3442 | 6992 | 10542 | V5 |
| Broad GPP | TFORF3353 | ZNF32 | NM_006973.2 | 3443 | 6993 | 10543 | V5 |
| Broad GPP | TFORF3354 | MXD1 | NM_002357.3 | 3444 | 6994 | 10544 | V5 |
| Broad GPP | TFORF3355 | FOXN2 | NM_002158.3 | 3445 | 6995 | 10545 | V5 |
| Genewiz | TFORF3356 | NRF1 | NM_001293163.1 | 3446 | 6996 | 10546 | None |
| Broad GPP | TFORF3357 | ZNF131 | NM_001330717.1 | 3447 | 6997 | 10547 | V5 |
| Broad GPP | TFORF3358 | STAT3 | NM_139276.2 | 3448 | 6998 | 10548 | V5 |
| Broad GPP | TFORF3359 | ZNF263 | NM_005741.4 | 3449 | 6999 | 10549 | V5 |
| Broad GPP | TFORF3360 | HOXB13 | NM_006361.5 | 3450 | 7000 | 10550 | V5 |
| Genewiz | TFORF3361 | MSC | NM_005098.3 | 3451 | 7001 | 10551 | None |
| Broad GPP | TFORF3362 | MYOG | NM_002479.5 | 3452 | 7002 | 10552 | V5 |
| Broad GPP | TFORF3363 | NR5A1 | NM_004959.4 | 3453 | 7003 | 10553 | V5 |
| Broad GPP | TFORF3364 | ZNF232 | NM_001320954.1 | 3454 | 7004 | 10554 | V5 |
| Broad GPP | TFORF3365 | FOXA1 | NM_004496.3 | 3455 | 7005 | 10555 | V5 |
| Broad GPP | TFORF3366 | FOXA3 | NM_004497.2 | 3456 | 7006 | 10556 | V5 |
| Broad GPP | TFORF3367 | ZNF281 | NM_012482.4 | 3457 | 7007 | 10557 | V5 |
| Broad GPP | TFORF3368 | FOXR1 | NM_181721.2 | 3458 | 7008 | 10558 | V5 |
| Broad GPP | TFORF3369 | BLZF1 | NM_001320973.1 | 3459 | 7009 | 10559 | V5 |
| Broad GPP | TFORF3370 | MYBL1 | NM_001144755.2 | 3460 | 7010 | 10560 | V5 |
| Broad GPP | TFORF3371 | MYBL1 | NM_001294282.1 | 3461 | 7011 | 10561 | V5 |
| Broad GPP | TFORF3372 | DAXX | NM_001141969.1 | 3462 | 7012 | 10562 | V5 |
| Broad GPP | TFORF3373 | ZNF101 | NM_001300949.1 | 3463 | 7013 | 10563 | V5 |
| Broad GPP | TFORF3374 | VAX2 | NM_012476.2 | 3464 | 7014 | 10564 | V5 |
| Broad GPP | TFORF3375 | TGIF2 | NM_021809.6 | 3465 | 7015 | 10565 | V5 |
| Broad GPP | TFORF3376 | TOX | NM_014729.2 | 3466 | 7016 | 10566 | V5 |
| Broad GPP | TFORF3377 | ZBTB39 | NM_014830.2 | 3467 | 7017 | 10567 | V5 |
| Broad GPP | TFORF3378 | ZSCAN21 | NM_145914.2 | 3468 | 7018 | 10568 | V5 |
| Broad GPP | TFORF3379 | ACTL6A | NM_004301.4 | 3469 | 7019 | 10569 | V5 |
| Broad GPP | TFORF3380 | ZNF3 | NM_001278291.1 | 3470 | 7020 | 10570 | V5 |
| Broad GPP | TFORF3381 | HNRNPK | NM_002140.4 | 3471 | 7021 | 10571 | V5 |
| Broad GPP | TFORF3382 | ZNF140 | NM_003440.3 | 3472 | 7022 | 10572 | V5 |
| Broad GPP | TFORF3383 | NFKBID | NM_139239.2 | 3473 | 7023 | 10573 | V5 |
| Broad GPP | TFORF3384 | HOXC10 | NM_017409.3 | 3474 | 7024 | 10574 | V5 |
| Broad GPP | TFORF3385 | ZIC3 | NM_003413.3 | 3475 | 7025 | 10575 | V5 |
| Broad GPP | TFORF3386 | IRF4 | NM_002460.3 | 3476 | 7026 | 10576 | V5 |
| Broad GPP | TFORF3387 | ZNF586 | NM_001077426.2 | 3477 | 7027 | 10577 | V5 |
| Broad GPP | TFORF3388 | YEATS4 | NM_006530.3 | 3478 | 7028 | 10578 | V5 |
| Broad GPP | TFORF3389 | ZNF580 | NM_207115.1 | 3479 | 7029 | 10579 | V5 |
| Broad GPP | TFORF3390 | SMAD5 | NM_005903.6 | 3480 | 7030 | 10580 | V5 |
| Broad GPP | TFORF3391 | CNBP | NM_001127196.1 | 3481 | 7031 | 10581 | V5 |
| Broad GPP | TFORF3392 | CNBP | NM_001127196.1 | 3482 | 7032 | 10582 | V5 |
| Broad GPP | TFORF3393 | ZBTB24 | NM_001164313.1 | 3483 | 7033 | 10583 | V5 |
| Broad GPP | TFORF3394 | ZBTB24 | NM_014797.2 | 3484 | 7034 | 10584 | V5 |
| Broad GPP | TFORF3395 | TBX22 | NM_016954.2 | 3485 | 7035 | 10585 | V5 |
| Genewiz | TFORF3396 | TARBP2 | NM_134323.1 | 3486 | 7036 | 10586 | None |
| Broad GPP | TFORF3397 | ZIK1 | NM_001321147.1 | 3487 | 7037 | 10587 | V5 |
| Broad GPP | TFORF3398 | HLF | NM_002126.4 | 3488 | 7038 | 10588 | V5 |
| Broad GPP | TFORF3399 | POU2F2 | NM_001247994.1 | 3489 | 7039 | 10589 | V5 |
| Broad GPP | TFORF3400 | GMEB1 | NM_024482.2 | 3490 | 7040 | 10590 | V5 |
| Broad GPP | TFORF3401 | GTF2B | NM_001514.5 | 3491 | 7041 | 10591 | V5 |
| Broad GPP | TFORF3402 | HSFY2 | XM_017030031.1 | 3492 | 7042 | 10592 | V5 |
| Broad GPP | TFORF3403 | ZNF488 | NM_001346933.1 | 3493 | 7043 | 10593 | V5 |
| Broad GPP | TFORF3404 | ZNF511 | NM_145806.3 | 3494 | 7044 | 10594 | V5 |
| Broad GPP | TFORF3405 | KCNIP4 | NM_025221.5 | 3495 | 7045 | 10595 | V5 |
| Broad GPP | TFORF3406 | ELOF1 | NM_032377.3 | 3496 | 7046 | 10596 | V5 |
| Broad GPP | TFORF3407 | ZNF397 | NM_032347.2 | 3497 | 7047 | 10597 | V5 |
| Broad GPP | TFORF3408 | ZNF286A | NM_001288648.1 | 3498 | 7048 | 10598 | V5 |
| Broad GPP | TFORF3409 | ZNF621 | NM_001098414.2 | 3499 | 7049 | 10599 | V5 |
| Broad GPP | TFORF3410 | ZKSCAN4 | NM_019110.4 | 3500 | 7050 | 10600 | V5 |
| Broad GPP | TFORF3411 | CRTC2 | NM_181715.2 | 3501 | 7051 | 10601 | V5 |
| Broad GPP | TFORF3412 | EGR1 | NM_001964.2 | 3502 | 7052 | 10602 | V5 |
| Broad GPP | TFORF3413 | ZFP82 | NM_133466.3 | 3503 | 7053 | 10603 | V5 |
| Broad GPP | TFORF3414 | NEUROG1 | NM_006161.2 | 3504 | 7054 | 10604 | V5 |
| Broad GPP | TFORF3415 | NKRF | NM_001173488.1 | 3505 | 7055 | 10605 | V5 |
| Broad GPP | TFORF3416 | ZNF410 | NM_001242926.1 | 3506 | 7056 | 10606 | V5 |
| Broad GPP | TFORF3417 | TCF7L2 | NM_001146284.1 | 3507 | 7057 | 10607 | V5 |
| Broad GPP | TFORF3418 | NFE2L2 | NM_006164.4 | 3508 | 7058 | 10608 | V5 |
| Broad GPP | TFORF3419 | ZNF562 | NM_001130031.1 | 3509 | 7059 | 10609 | V5 |
| Broad GPP | TFORF3420 | SNAPC1 | NM_003082.3 | 3510 | 7060 | 10610 | V5 |
| Broad GPP | TFORF3421 | YBX2 | NM_015982.3 | 3511 | 7061 | 10611 | V5 |
| Broad GPP | TFORF3422 | HES6 | NM_018645.5 | 3512 | 7062 | 10612 | V5 |
| Broad GPP | TFORF3423 | ZNF345 | NM_003419.4 | 3513 | 7063 | 10613 | V5 |
| Broad GPP | TFORF3424 | ZNF57 | NM_173480.2 | 3514 | 7064 | 10614 | V5 |
| Broad GPP | TFORF3425 | ZNF559 | NM_032497.2 | 3515 | 7065 | 10615 | V5 |
| Broad GPP | TFORF3426 | ZNF350 | NM_021632.3 | 3516 | 7066 | 10616 | V5 |
| Broad GPP | TFORF3427 | NFIB | NM_005596.3 | 3517 | 7067 | 10617 | V5 |
| Broad GPP | TFORF3428 | TRIM27 | NM_006510.4 | 3518 | 7068 | 10618 | V5 |
| Broad GPP | TFORF3429 | ZNF302 | NM_001289186.1 | 3519 | 7069 | 10619 | V5 |
| Broad GPP | TFORF3430 | FXN | NM_000144.4 | 3520 | 7070 | 10620 | V5 |
| Broad GPP | TFORF3431 | NR3C2 | NM_000901.4 | 3521 | 7071 | 10621 | V5 |
| Broad GPP | TFORF3432 | NR3C1 | NM_000176.2 | 3522 | 7072 | 10622 | V5 |
| Broad GPP | TFORF3433 | DEK | NM_003472.3 | 3523 | 7073 | 10623 | V5 |
| Broad GPP | TFORF3434 | ZNF37A | NM_001178101.1 | 3524 | 7074 | 10624 | V5 |
| Broad GPP | TFORF3435 | SUPT5H | NM_003169.3 | 3525 | 7075 | 10625 | V5 |
| Broad GPP | TFORF3436 | TOX4 | NM_014828.3 | 3526 | 7076 | 10626 | V5 |
| Broad GPP | TFORF3437 | RBAK | NM_021163.3 | 3527 | 7077 | 10627 | V5 |
| Broad GPP | TFORF3438 | GCM1 | NM_003643.3 | 3528 | 7078 | 10628 | V5 |
| Broad GPP | TFORF3439 | FOXI1 | NM_144769.2 | 3529 | 7079 | 10629 | V5 |
| Broad GPP | TFORF3440 | ING1 | NM_198219.2 | 3530 | 7080 | 10630 | V5 |
| Broad GPP | TFORF3441 | MNAT1 | NM_002431.3 | 3531 | 7081 | 10631 | V5 |
| Broad GPP | TFORF3442 | ZNF177 | NM_003451.2 | 3532 | 7082 | 10632 | V5 |
| Broad GPP | TFORF3443 | SP4 | NM_003112.4 | 3533 | 7083 | 10633 | V5 |
| Broad GPP | TFORF3444 | KCNIP2 | NM_173192.2 | 3534 | 7084 | 10634 | V5 |
| Broad GPP | TFORF3445 | PTTG1 | NM_004219.3 | 3535 | 7085 | 10635 | V5 |
| Broad GPP | TFORF3446 | ZNF786 | NM_152411.3 | 3536 | 7086 | 10636 | V5 |
| Broad GPP | TFORF3447 | MYCBP | NM_012333.4 | 3537 | 7087 | 10637 | V5 |
| Broad GPP | TFORF3448 | TP63 | NM_003722.4 | 3538 | 7088 | 10638 | V5 |
| Broad GPP | TFORF3449 | DMRT1 | NM_021951.2 | 3539 | 7089 | 10639 | V5 |
| Broad GPP | TFORF3450 | ELK1 | NM_001114123.2 | 3540 | 7090 | 10640 | V5 |
| Broad GPP | TFORF3451 | ZNF366 | NM_152625.2 | 3541 | 7091 | 10641 | V5 |
| Broad GPP | TFORF3452 | ZNF460 | NM_006635.3 | 3542 | 7092 | 10642 | V5 |
| Broad GPP | TFORF3453 | PREB | NM_013388.5 | 3543 | 7093 | 10643 | V5 |
| Broad GPP | TFORF3454 | ZNF653 | NM_138783.3 | 3544 | 7094 | 10644 | V5 |
| Broad GPP | TFORF3455 | ZNF513 | NM_144631.5 | 3545 | 7095 | 10645 | V5 |
| Broad GPP | TFORF3456 | ZNF426 | NM_024106.2 | 3546 | 7096 | 10646 | V5 |
| Broad GPP | TFORF3457 | IRF9 | NM_006084.4 | 3547 | 7097 | 10647 | V5 |
| Broad GPP | TFORF3458 | ZNF556 | NM_001300843.1 | 3548 | 7098 | 10648 | V5 |
| Broad GPP | TFORF3459 | HMGB2 | NM_002129.3 | 3549 | 7099 | 10649 | V5 |
| Broad GPP | TFORF3460 | PSMB1 | NM_002793.3 | 3550 | 7100 | 10650 | V5 |
| Broad GPP | TFORF3461 | ETV1 | NM_004956.4 | 3551 | 7101 | 10651 | V5 |
| Broad GPP | TFORF3462 | BRPF1 | NM_001003694.1 | 3552 | 7102 | 10652 | V5 |
| Broad GPP | TFORF3463 | ETV7 | NM_016135.3 | 3553 | 7103 | 10653 | V5 |
| Broad GPP | TFORF3464 | KLF7 | NM_001270942.1 | 3554 | 7104 | 10654 | V5 |
| Broad GPP | TFORF3465 | ZBTB48 | NM_001278647.1 | 3555 | 7105 | 10655 | V5 |
| Broad GPP | TFORF3466 | SIM2 | NM_009586.4 | 3556 | 7106 | 10656 | V5 |
| Broad GPP | TFORF3467 | SUPT4H1 | NM_003168.2 | 3557 | 7107 | 10657 | V5 |
| Broad GPP | TFORF3468 | PKNOX1 | NM_004571.4 | 3558 | 7108 | 10658 | V5 |
| Broad GPP | TFORF3469 | ZNF438 | NM_182755.2 | 3559 | 7109 | 10659 | V5 |
| Broad GPP | TFORF3470 | ZNF726 | NM_001348689.1 | 3560 | 7110 | 10660 | V5 |
| Broad GPP | TFORF3471 | ZNF670 | NM_033213.4 | 3561 | 7111 | 10661 | V5 |
| Broad GPP | TFORF3472 | NFATC3 | NM_173165.2 | 3562 | 7112 | 10662 | V5 |
| Broad GPP | TFORF3473 | RUNX1 | NM_001754.4 | 3563 | 7113 | 10663 | V5 |
| Broad GPP | TFORF3474 | ZNF41 | NM_001324150.1 | 3564 | 7114 | 10664 | V5 |
| Broad GPP | TFORF3475 | IL18 | NM_001562.3 | 3565 | 7115 | 10665 | V5 |
| Broad GPP | TFORF3476 | SNAI1 | NM_005985.3 | 3566 | 7116 | 10666 | V5 |
| Broad GPP | TFORF3477 | POU5F1 | NM_002701.5 | 3567 | 7117 | 10667 | V5 |
| Broad GPP | TFORF3478 | FOXD4 | NM_207305.4 | 3568 | 7118 | 10668 | V5 |
| Broad GPP | TFORF3479 | ZNF132 | NM_003433.3 | 3569 | 7119 | 10669 | V5 |
| Broad GPP | TFORF3480 | ZNF785 | NM_152458.6 | 3570 | 7120 | 10670 | V5 |
| Broad GPP | TFORF3481 | ZNF597 | NM_152457.2 | 3571 | 7121 | 10671 | V5 |
| Broad GPP | TFORF3482 | SOX2 | NM_003106.3 | 3572 | 7122 | 10672 | V5 |
| Genewiz | TFORF3483 | PBX4 | NM_025245.2 | 3573 | 7123 | 10673 | None |
| Broad GPP | TFORF3484 | ZNF398 | NM_170686.2 | 3574 | 7124 | 10674 | V5 |
| Broad GPP | TFORF3485 | ATF2 | NM_001880.3 | 3575 | 7125 | 10675 | V5 |
| Broad GPP | TFORF3486 | PRDM1 | NM_182907.2 | 3576 | 7126 | 10676 | V5 |
| Broad GPP | TFORF3487 | NFATC1 | NM_172390.2 | 3577 | 7127 | 10677 | V5 |
| Broad GPP | TFORF3488 | ZNF169 | XM_017014364.1 | 3578 | 7128 | 10678 | V5 |
| Broad GPP | TFORF3489 | IKZF1 | NM_001291837.1 | 3579 | 7129 | 10679 | V5 |
| Broad GPP | TFORF3490 | TCF23 | NM_175769.2 | 3580 | 7130 | 10680 | V5 |
| Broad GPP | TFORF3491 | NR113 | NM_001077480.2 | 3581 | 7131 | 10681 | None |
| Broad GPP | TFORF3492 | RELB | NM_006509.3 | 3582 | 7132 | 10682 | V5 |
| Broad GPP | TFORF3493 | ZNF791 | NM_153358.2 | 3583 | 7133 | 10683 | V5 |
| Broad GPP | TFORF3494 | OSR2 | NM_053001.3 | 3584 | 7134 | 10684 | V5 |
| Broad GPP | TFORF3495 | CLOCK | NM_001267843.1 | 3585 | 7135 | 10685 | V5 |
| Broad GPP | TFORF3496 | REL | NM_001291746.1 | 3586 | 7136 | 10686 | V5 |
| Broad GPP | TFORF3497 | ZNF280C | NM_017666.4 | 3587 | 7137 | 10687 | V5 |
| Broad GPP | TFORF3498 | APEX1 | NM_001641.3 | 3588 | 7138 | 10688 | V5 |
| Broad GPP | TFORF3499 | RARA | NM_001145301.2 | 3589 | 7139 | 10689 | V5 |
| Broad GPP | TFORF3500 | BATF3 | NM_018664.2 | 3590 | 7140 | 10690 | V5 |
| Broad GPP | TFORF3501 | CERS6 | NM_001256126.1 | 3591 | 7141 | 10691 | V5 |
| Broad GPP | TFORF3502 | VENTX | NM_014468.3 | 3592 | 7142 | 10692 | V5 |
| Broad GPP | TFORF3503 | CDK7 | NM_001799.3 | 3593 | 7143 | 10693 | V5 |
| Broad GPP | TFORF3504 | ZNF543 | NM_213598.3 | 3594 | 7144 | 10694 | V5 |
| Broad GPP | TFORF3505 | KLF12 | NM_007249.4 | 3595 | 7145 | 10695 | V5 |
| Broad GPP | TFORF3506 | LCOR | NM_001170766.1 | 3596 | 7146 | 10696 | V5 |
| Broad GPP | TFORF3507 | NOCT | NM_012118.3 | 3597 | 7147 | 10697 | V5 |
| Broad GPP | TFORF3508 | ZNF134 | NM_003435.4 | 3598 | 7148 | 10698 | V5 |
| Broad GPP | TFORF3509 | GATAD2B | NM_020699.3 | 3599 | 7149 | 10699 | V5 |
| Broad GPP | TFORF3510 | TCF25 | NM_014972.2 | 3600 | 7150 | 10700 | V5 |
| Broad GPP | TFORF3511 | SOX14 | NM_004189.3 | 3601 | 7151 | 10701 | V5 |
| Broad GPP | TFORF3512 | SOX14 | NM_004189.3 | 3602 | 7152 | 10702 | V5 |
| Broad GPP | TFORF3513 | GLMP | NM_144580.2 | 3603 | 7153 | 10703 | V5 |
| Broad GPP | TFORF3514 | PURB | NM_033224.4 | 3604 | 7154 | 10704 | V5 |
| Broad GPP | TFORF3515 | HDGF | NM_004494.2 | 3605 | 7155 | 10705 | V5 |
| Broad GPP | TFORF3516 | RNF138 | NM_016271.4 | 3606 | 7156 | 10706 | V5 |
| Broad GPP | TFORF3517 | SOX12 | NM_006943.3 | 3607 | 7157 | 10707 | V5 |
| Broad GPP | TFORF3518 | NHLH2 | NM_001111061.1 | 3608 | 7158 | 10708 | V5 |
| Broad GPP | TFORF3519 | TCF4 | NM_003199.2 | 3609 | 7159 | 10709 | None |
| Broad GPP | TFORF3520 | TCF4 | NM_001083962.1 | 3610 | 7160 | 10710 | V5 |
| Broad GPP | TFORF3521 | THAP8 | NM_152658.2 | 3611 | 7161 | 10711 | V5 |
| Broad GPP | TFORF3522 | POU6F1 | XM_017019524.1 | 3612 | 7162 | 10712 | V5 |
| Broad GPP | TFORF3523 | ZNF473 | NM_015428.3 | 3613 | 7163 | 10713 | V5 |
| Broad GPP | TFORF3524 | MAEL | NM_032858.2 | 3614 | 7164 | 10714 | V5 |
| Broad GPP | TFORF3525 | THAP2 | NM_031435.3 | 3615 | 7165 | 10715 | V5 |
| Broad GPP | TFORF3526 | THAP12 | NM_004705.3 | 3616 | 7166 | 10716 | V5 |
| Broad GPP | TFORF3527 | ERF | NM_006494.3 | 3617 | 7167 | 10717 | V5 |
| Broad GPP | TFORF3528 | NME2 | NM_001018138.1 | 3618 | 7168 | 10718 | V5 |
| Broad GPP | TFORF3529 | VDR | NM_000376.2 | 3619 | 7169 | 10719 | V5 |
| Broad GPP | TFORF3530 | SSRP1 | NM_003146.2 | 3620 | 7170 | 10720 | V5 |
| Broad GPP | TFORF3531 | HMGN3 | NM_138730.2 | 3621 | 7171 | 10721 | V5 |
| Broad GPP | TFORF3532 | LIN28B | NM_001004317.3 | 3622 | 7172 | 10722 | V5 |
| Broad GPP | TFORF3533 | CDIP1 | NM_013399.2 | 3623 | 7173 | 10723 | None |
| Broad GPP | TFORF3534 | BORCS8- | NM_005919.3 | 3624 | 7174 | 10724 | V5 |
| | | MEF2B | | | | | |
| Broad GPP | TFORF3535 | MLLT10 | NM_001195627.1 | 3625 | 7175 | 10725 | V5 |
| Broad GPP | TFORF3536 | HOXD4 | NM_014621.2 | 3626 | 7176 | 10726 | V5 |
| Broad GPP | TFORF3537 | TSC22D4 | NM_001303043.1 | 3627 | 7177 | 10727 | V5 |
| Broad GPP | TFORF3538 | HOXD10 | NM_002148.3 | 3628 | 7178 | 10728 | V5 |
| Broad GPP | TFORF3539 | ZNF747 | NM_023931.3 | 3629 | 7179 | 10729 | V5 |
| Broad GPP | TFORF3540 | TAL2 | NM_005421.2 | 3630 | 7180 | 10730 | V5 |
| Broad GPP | TFORF3541 | THAP6 | NM_144721.5 | 3631 | 7181 | 10731 | V5 |
| Broad GPP | TFORF3542 | ZNF720 | NM_001130913.1 | 3632 | 7182 | 10732 | V5 |
| Broad GPP | TFORF3543 | CSDC2 | NM_014460.3 | 3633 | 7183 | 10733 | V5 |
| Broad GPP | TFORF3544 | NR2E1 | NM_003269.4 | 3634 | 7184 | 10734 | V5 |
| Broad GPP | TFORF3545 | PIAS1 | NM_016166.2 | 3635 | 7185 | 10735 | V5 |
| Broad GPP | TFORF3546 | TSC22D3 | NM_001015881.1 | 3636 | 7186 | 10736 | V5 |
| Broad GPP | TFORF3547 | CBFB | NM_001755.2 | 3637 | 7187 | 10737 | V5 |
| Broad GPP | TFORF3548 | THAP10 | NM_020147.3 | 3638 | 7188 | 10738 | V5 |
| Neither | TFORF3549 | GFP | — | 3639 | 7189 | 10739 | None |
| Neither | TFORF3550 | mCherry | — | 3640 | 7190 | 10740 | None |

TABLE 4
Transcription Factor Enrichment in Radial Glia by Flow-FISH

| | | | Fold |
|---|---|---|---|
| Gene | Refseq ID | ORF ID | enrichment |
| RFX4 | NM_001206691 | pORF0063 | 4.592962779 |
| NFIC | NM_005597 | pORF0048 | 4.456389351 |
| OTX1 | NM_014562 | pORF0051 | 4.101884122 |
| NOTCH2 | NM_024408 | pORF0088 | 3.943317106 |
| NFIC | NM_001245004 | pORF0049 | 3.902336252 |
| LHX2 | NM_004789 | pORF0037 | 3.184572774 |
| NFIB | NM_001190737 | pORF0047 | 2.992118862 |
| FOS | NM_005252 | pORF0021 | 2.819461324 |
| EOMES | NM_005442 | pORF0018 | 2.493885045 |
| NFIB | NM_005596 | pORF0046 | 2.118506211 |
| HES1 | NM_005524 | pORF0029 | 2.062443348 |
| SOX9 | NM_000346 | pORF0070 | 2.037365883 |
| BRIP1 | NM_032043 | pORF0083 | 1.983878329 |
| ZFP36L1 | NM_001244701 | pORF0082 | 1.732874565 |
| PAX6 | NM_001310159 | pORF0052 | 1.729014559 |
| SOX2 | NM_003106 | pORF0068 | 1.703600734 |
| MEIS1 | NM_002398 | pORF0040 | 1.63016496 |
| PAX6 | NM_000280 | pORF0053 | 1.621301866 |
| E2F7 | NM_203394 | pORF0011 | 1.445290255 |
| SOX1 | NM_005986 | pORF0066 | 1.414928718 |
| BCL11A | NM_022893 | pORF0004 | 1.366033006 |
| TCF12 | NM_207037 | pORF0075 | 1.338644735 |
| HELLS | NM_001289068 | pORF0027 | 1.314452769 |
| NOTCH1 | NM_017617 | pORF0087 | 1.282543236 |
| ASCL1 | NM_004316 | pORF0002 | 1.257269521 |
| ID4 | NM_001546 | pORF0034 | 1.242259505 |
| RFX2 | NM_000635 | pORF0062 | 1.156789924 |
| ID3 | NM_002167 | pORF0033 | 1.13013705 |
| FOXN4 | NM_213596 | pORF0025 | 1.118252693 |
| FANCD2 | NM_001018115 | pORF0085 | 1.105106279 |
| CXXC1 | NM_014593 | pORF0008 | 1.10158182 |
| RAD54L | NM_001142548 | pORF0059 | 1.100049356 |
| PLK4 | NM_001190801 | pORF0056 | 1.087864401 |
| MXD3 | NM_031300 | pORF0041 | 1.046651367 |
| SMARCC1 | NM_003074 | pORF0093 | 1.013071774 |
| CHAF1A | NM_005483 | pORF0084 | 0.987758678 |
| STAT3 | NM_213662 | pORF0073 | 0.982494628 |
| EMX2 | NM_004098 | pORF0015 | 0.980888997 |
| RFX4 | NM_032491 | pORF0064 | 0.97115811 |
| STAT3 | NM_003150 | pORF0072 | 0.940727106 |
| TEAD2 | NM_003598 | pORF0077 | 0.93305152 |
| RCOR2 | NM_173587 | pORF0060 | 0.92635635 |
| MYBL2 | NM_001278610 | pORF0043 | 0.921257809 |
| SSRP1 | NM_003146 | pORF0071 | 0.888548451 |
| TCF12 | NM_207038 | pORF0074 | 0.887183472 |
| HELLS | NM_001289073 | pORF0028 | 0.848309668 |
| CDK1 | NM_001786 | pORF0005 | 0.84539673 |
| HMGB1 | NM_002128 | pORF0030 | 0.82087272 |
| ENO1 | NM_001428 | pORF0017 | 0.810855094 |
| CENPA | NM_001042426 | pORF0007 | 0.800609156 |
| TRIM28 | NM_005762 | pORF0079 | 0.795174082 |
| H2AFX | NM_002105 | pORF0026 | 0.779428312 |
| EOMES | NM_001278183 | pORF0019 | 0.775123473 |
| ENO1 | NM_001201483 | pORF0016 | 0.752089926 |
| PLK4 | NM_001190799 | pORF0055 | 0.747675661 |
| TCF7L2 | NM_001146286 | pORF0076 | 0.7461346 |
| MXD3 | NM_001142935 | pORF0042 | 0.742008629 |
| YAP1 | NM_001195045 | pORF0081 | 0.733973908 |
| KLF15 | NM_014079 | pORF0036 | 0.724234171 |
| HMGB2 | NM_002129 | pORF0031 | 0.715645669 |
| MAZ | NM_001276275 | pORF0039 | 0.712224529 |
| SMAD1 | NM_001003688 | pORF0065 | 0.691823503 |
| PRMT7 | NM_001184824 | pORF0058 | 0.685275524 |
| E2F2 | NM_004091 | pORF0010 | 0.68015417 |
| GLI3 | NM_000168 | pORF0086 | 0.676437305 |
| PAX6 | NM_001310160 | pORF0054 | 0.670896717 |
| RFX2 | NM_134433 | pORF0061 | 0.662831174 |
| BCL11A | NM_138559 | pORF0003 | 0.653637098 |
| SOX11 | NM_003108 | pORF0067 | 0.648216511 |
| FOXG1 | NM_005249 | pORF0022 | 0.643117047 |
| POU3F2 | NM_005604 | pORF0057 | 0.638941761 |
| HOPX | NM_139212 | pORF0032 | 0.63780869 |
| UHRF1 | NM_001290052 | pORF0080 | 0.634190647 |
| TEAD2 | NM_001256662 | pORF0078 | 0.622268143 |
| CDK1 | NM_001170407 | pORF0006 | 0.618841246 |
| MAZ | NM_001276276 | pORF0038 | 0.601623712 |
| ARX | NM_139058 | pORF0001 | 0.599805685 |
| HES5 | NM_001010926 | pORF0094 | 0.599030362 |
| FOXM1 | NM_202003 | pORF0024 | 0.592181164 |
| EGR1 | NM_001964 | pORF0013 | 0.587722694 |
| E2F1 | NM_005225 | pORF0009 | 0.554009014 |
| EMX2 | NM_001165924 | pORF0014 | 0.54756126 |
| E2F8 | NM_001256372 | pORF0012 | 0.531116602 |
| NFATC4 | NM_001288802 | pORF0044 | 0.529705034 |
| NR1D1 | NM_021724 | pORF0050 | 0.518527509 |
| FOXM1 | NM_001243089 | pORF0023 | 0.50781922 |
| FEZF2 | NM_018008 | pORF0020 | 0.427110797 |
| NFATC4 | NM_001198966 | pORF0045 | 0.420447714 |
| INSM1 | NM_002196 | pORF0035 | 0.41826746 |
| SOX4 | NM_003107 | pORF0069 | 0.376315081 |

TABLE 5
Number of cells analyzed using single-cell RNA-seq in each
biorep of spontaneously differentiated cells. Number of
cells used in the analyses after filtering using Seurat.

| Condition | Number of cells |
|---|---|
| RFX4 S1 | 6,120 |
| RFX4 S2 | 7,109 |
| NFIB S1 | 6,343 |
| NFIB S2 | 7,935 |
| PAX6 S1 | 7,557 |
| PAX6 S1 | 6,417 |
| ASCL1 S1 | 6,652 |
| ASCL1 S2 | 4,231 |

TABLE 6
Cluster marker genes for each scRNA-seq dataset. (A) scRNA-seq data from 53,113 cells that have been
spontaneously differentiated from iNPs for 8 weeks. iNPs were derived using RFX4, NFIB, ASCL1, or PAX6
with n = biological replicates per TF. (B) UMAP clustering of scRNA-seq data from 42,780 iNPs derived
using three iNP differentiation methods. iNP differentiation methods included RFX4 overexpression with
dual SMAD inhibition (15,211 cells), embryoid body formation (11,148 cells), and dual SMAD inhibition
(16,421 cells). Data represents n = batch replicates per method. (C) UMAP clustering of scRNA-seq data from
26,111 cells that have been spontaneously differentiated from iNPs. iNPs were produced by combining RFX4
overexpression with dual SMAD inhibition and spontaneously differentiated for 4 or 8 weeks. Data represents
n = biological replicates per timepoint. For each dataset, the top 30 marker genes and associated p-values
for each cluster were identified using the scanpy.tl.rank_gene_groups function.
Table 6A

| Cluster 0 | Cluster 0 | Cluster 1 | Cluster 1 | Cluster 2 | Cluster 2 | Cluster 3 | Cluster 3 |
|---|---|---|---|---|---|---|---|
| Gene | P-value | Gene | P-value | Gene | P-value | Gene | P-value |
| DAPL1 | 0 | RPLP0 | 0 | SFRP2 | 0 | COL1A1 | 0 |
| SFRP2 | 0 | RPS3A | 0 | ARL4A | 0 | COL1A2 | 0 |
| PTH2 | 0 | EEF1A1 | 0 | PCDH9 | 0 | LGALS1 | 0 |
| CYP1B1 | 0 | RPL34 | 3.04E−302 | NR2F1 | 0 | COL3A1 | 0 |
| RAX | 0 | RPL26 | 2.91E−298 | RPL5 | 0 | MMP2 | 0 |
| HMX1 | 0 | RPL7 | 4.38E−269 | HSD17B2 | 0 | COL6A2 | 0 |
| CRABP1 | 0 | RPL35A | 4.26E−255 | DAPL1 | 0 | LUM | 0 |
| RPS7 | 0 | RPL21 | 3.17E−228 | RPS3A | 0 | FBLN1 | 0 |
| RPL7 | 0 | RPL31 | 6.96E−225 | CPE | 0 | COL9A3 | 0 |
| SPP1 | 0 | RPS17 | 3.04E−210 | PAX6 | 0 | RPL10 | 0 |
| SIX6 | 0 | RPS15A | 1.89E−203 | FLRT3 | 0 | SERPINH1 | 0 |
| NUDT4 | 0 | RPS6 | 1.50E−197 | HMX1 | 0 | TGFBI | 0 |
| DKK3 | 0 | RPS12 | 5.31E−191 | MAB21L1 | 0 | DCN | 0 |
| COL2A1 | 0 | RPL9 | 5.37E−189 | RPL35A | 0 | IGF2 | 0 |
| NAP1L1 | 0 | RPL6 | 1.37E−184 | ZFP36L2 | 0 | SPARC | 0 |
| MIAT | 0 | RPL5 | 2.29E−184 | EFNA5 | 0 | IGFBP4 | 0 |
| TRH | 0 | RPS24 | 1.12E−178 | RPS7 | 0 | COL5A2 | 0 |
| ZFP36L2 | 0 | RPL11 | 1.10E−172 | MT-ND2 | 0 | RPL28 | 0 |
| LMAN1 | 0 | RPL12 | 7.56E−153 | SIX6 | 0 | TPT1 | 0 |
| RAB31 | 0 | RPS13 | 4.55E−150 | TKT | 0 | MGP | 0 |
| RPS3A | 0 | RPL24 | 9.20E−149 | COL9A1 | 0 | IL11RA | 0 |
| TPPP3 | 0 | RPS8 | 8.46E−147 | RP4- | 0 | COL5A1 | 0 |
| | | | | 665J23.1 | | | |
| SOX2 | 0 | RPL32 | 5.53E−146 | RPL7 | 0 | TWIST1 | 0 |
| MAB21L1 | 0 | RPL30 | 6.38E−144 | RPL30 | 0 | IFITM3 | 0 |
| RPL30 | 0 | RPS25 | 5.76E−143 | RPL39 | 0 | CEBPD | 0 |
| MSI2 | 0 | RPL39 | 6.53E−139 | RPL9 | 0 | FTL | 0 |
| ALDH1A1 | 0 | RPS7 | 9.28E−139 | RPL34 | 0 | MFAP2 | 0 |
| LHX2 | 0 | RPLP1 | 1.98E−137 | QPRT | 0 | PCOLCE | 0 |
| PAX6 | 0 | RPS4X | 1.50E−136 | HSP90AB1 | 0 | COL6A1 | 0 |
| TTYH1 | 0 | RPL41 | 1.95E−135 | EIF3E | 0 | SELM | 0 |

| Cluster 4 | Cluster 4 | Cluster 5 | Cluster 5 | Cluster 6 | Cluster 6 | Cluster 7 | Cluster 7 |
|---|---|---|---|---|---|---|---|
| Gene | P-value | Gene | P-value | Gene | P-value | Gene | P-value |
| PMEL | 0 | LUM | 0 | TUBB2B | 0 | LGALS1 | 0 |
| GJA1 | 0 | MGP | 0 | TUBA1A | 0 | BGN | 0 |
| APOE | 0 | DCN | 0 | BASP1 | 0 | SPARC | 0 |
| SLC2A1 | 0 | COL1A1 | 0 | STMN2 | 0 | COL1A1 | 0 |
| ARL4A | 0 | COL1A2 | 0 | MLLT11 | 0 | TAGLN | 0 |
| HSD17B2 | 0 | COL3A1 | 0 | HN1 | 0 | COL1A2 | 0 |
| NKAIN4 | 0 | BGN | 0 | SOX4 | 0 | SERPINH1 | 0 |
| PAX6 | 0 | FBLN1 | 0 | DCX | 0 | COL3A1 | 0 |
| TRPM3 | 0 | PCOLCE | 0 | MAP1B | 0 | POSTN | 0 |
| ALDH1A1 | 0 | MMP2 | 0 | TUBB2A | 0 | TGFBI | 0 |
| TSC22D1 | 0 | EGFL6 | 0 | CRMP1 | 0 | RPL28 | 0 |
| TMEFF2 | 0 | LGALS1 | 0 | RAB3A | 0 | MGP | 0 |
| NPC2 | 0 | SPARC | 0 | STMN4 | 0 | CCDC80 | 0 |
| PTGDS | 0 | MFAP4 | 0 | BEX1 | 0 | NUPR1 | 0 |
| SDCBP | 0 | IGFBP4 | 0 | MARCKSL1 | 0 | IGFBP7 | 0 |
| MT-CYB | 0 | COL6A2 | 0 | MALAT1 | 0 | FHL2 | 0 |
| APLP2 | 0 | DKK2 | 0 | GAP43 | 0 | LUM | 0 |
| DCT | 0 | CRABP2 | 0 | TAGLN3 | 0 | MMP2 | 0 |
| LINC01474 | 0 | COL5A2 | 0 | GNG3 | 0 | RPL12 | 0 |
| SERPINF1 | 0 | SERPINH1 | 0 | TUBB | 0 | S100A11 | 0 |
| DCBLD2 | 0 | TPT1 | 0 | NSG1 | 0 | TPM1 | 0 |
| ELP4 | 0 | TCF4 | 0 | ELAVL3 | 0 | TPT1 | 0 |
| MT-CO3 | 0 | TGFBI | 0 | CD24 | 0 | DCN | 0 |
| MITF | 0 | CDC42EP5 | 0 | PCSK1N | 0 | ANXA2 | 0 |
| SNCA | 0 | ITM2C | 0 | STMN1 | 0 | PTRF | 0 |
| PCDH9 | 0 | FCGRT | 0 | APLP1 | 0 | SFRP4 | 0 |
| LMO4 | 0 | MFAP2 | 0 | TTC3 | 0 | NNMT | 0 |
| GAS1 | 0 | GLT8D2 | 0 | NREP | 0 | TPM2 | 0 |
| MT-CO2 | 0 | RPL10 | 0 | MIAT | 0 | PPIB | 0 |
| SPOCK1 | 0 | MXRA8 | 0 | VAMP2 | 0 | FN1 | 0 |

| Cluster 8 | Cluster 8 | Cluster 9 | Cluster 9 | Cluster 10 | Cluster 10 | Cluster 11 | Cluster 11 |
|---|---|---|---|---|---|---|---|
| Gene | P-value | Gene | P-value | Gene | P-value | Gene | P-value |
| HMGB2 | 0 | ARL4A | 0 | PTN | 0 | GPM6B | 0 |
| UBE2C | 0 | HSD17B2 | 0 | GPM6B | 0 | TTYH1 | 0 |
| TUBA1B | 0 | GJA1 | 0 | VIM | 0 | SOX2 | 0 |
| SMC4 | 0 | TSC22D1 | 0 | TUBA1A | 0 | CKB | 0 |
| BIRC5 | 0 | PAX6 | 0 | C1orf61 | 0 | TUBA1A | 0 |
| CKS1B | 0 | EFNA5 | 0 | CRYAB | 0 | ID4 | 0 |
| TOP2A | 0 | TRPM3 | 0 | B2M | 0 | VIM | 0 |
| NUSAP1 | 0 | NR2F1 | 0 | C1QL1 | 0 | FGFBP3 | 0 |
| PTTG1 | 0 | CFI | 0 | CD99 | 0 | PTN | 0 |
| H2AFZ | 0 | PCDH9 | 0 | CLU | 0 | TUBB2B | 0 |
| CENPF | 0 | PMEL | 0 | FEZ1 | 0 | C1orf61 | 0.00E+00 |
| AURKB | 0 | NKAIN4 | 0 | GFAP | 0 | PDLIM3 | 1.57E−305 |
| CKS2 | 0 | SFRP1 | 6.33E−305 | CNN3 | 7.08E−308 | NES | 2.54E−299 |
| UBE2T | 0 | CPE | 9.20E−296 | TUBB2B | 6.03E−306 | HES5 | 4.39E−290 |
| MAD2L1 | 0 | SNCA | 6.73E−290 | NGFRAP1 | 1.47E−301 | NTRK2 | 2.83E−261 |
| MKI67 | 0 | ZIC1 | 1.86E−289 | PCSK1N | 6.61E−299 | CD99 | 6.71E−260 |
| FAM64A | 0 | MAL | 4.13E−273 | S100B | 1.49E−280 | CXCR4 | 2.49E−257 |
| UBE2S | 0 | TKT | 1.68E−270 | PEA15 | 5.42E−277 | METRN | 1.70E−250 |
| CDK1 | 0 | SLC2A1 | 1.50E−264 | LYPD1 | 3.27E−271 | FEZ1 | 1.13E−243 |
| TUBB4B | 0 | COL9A1 | 6.97E−256 | ID3 | 1.38E−266 | MAP1B | 1.15E−240 |
| CCNA2 | 0 | MAB21L1 | 1.10E−249 | CFL1 | 2.09E−256 | CDKN1A | 1.70E−237 |
| PBK | 0 | ALDH1A1 | 1.64E−249 | LAPTM4B | 1.88E−254 | RPS27L | 1.99E−237 |
| NUCKS1 | 0 | APOE | 5.27E−245 | ID4 | 6.64E−253 | FDFT1 | 6.39E−232 |
| CDKN3 | 0 | NPC2 | 1.13E−240 | ACTB | 7.32E−253 | TUBB2A | 6.18E−223 |
| TPX2 | 0 | COL9A2 | 2.25E−234 | METRN | 9.33E−247 | EDNRB | 8.89E−219 |
| CCNB2 | 0 | ITM2B | 9.82E−216 | FHL1 | 4.13E−235 | LINC00461 | 5.05E−211 |
| PRC1 | 0 | RPL5 | 3.01E−214 | MYL6 | 2.88E−229 | ID3 | 1.58E−204 |
| GTSE1 | 0 | TMSB4X | 2.04E−207 | IFI6 | 8.71E−223 | DOK5 | 5.02E−203 |
| KPNA2 | 0 | ACTG1 | 4.12E−207 | FXYD6 | 4.07E−222 | PON2 | 1.44E−200 |
| TYMS | 0 | FAM84A | 2.93E−206 | TTYH1 | 3.24E−221 | NLRP1 | 2.01E−197 |

| Cluster 12 | Cluster 12 | Cluster 13 | Cluster 13 | Cluster 14 | Cluster 14 | Cluster 15 | Cluster 15 |
|---|---|---|---|---|---|---|---|
| Gene | P-value | Gene | P-value | Gene | P-value | Gene | P-value |
| OSR2 | 0 | TUBA1B | 0 | MIAT | 0 | S100A11 | 0 |
| FBLN1 | 0 | UBE2C | 0 | RBP1 | 0 | KRT19 | 0 |
| COL3A1 | 0 | HMGB2 | 0 | MAP1B | 0 | KRT17 | 0 |
| TGFBI | 0 | PTTG1 | 0 | STMN2 | 0 | S100A10 | 0 |
| COL1A1 | 0 | HMGB1 | 0 | PCBP4 | 0 | HSPB1 | 0 |
| ALX1 | 0 | H2AFZ | 0 | MLLT11 | 0 | S100A14 | 0 |
| ITM2A | 0 | CKS1B | 0 | TUBA1A | 0 | KRT8 | 4.83E−282 |
| NBL1 | 0 | BIRC5 | 0 | TAGLN3 | 0 | PERP | 5.42E−246 |
| RPL15 | 0 | TUBB | 0 | SH3BGRL3 | 0 | KRT18 | 7.28E−245 |
| COL1A2 | 0 | STMN1 | 1.08E−267 | FAM57B | 0 | PDLIM1 | 5.00E−239 |
| LGALS1 | 0 | HMGN2 | 6.15E−265 | CRABP1 | 0 | FTH1 | 1.49E−204 |
| POSTN | 0 | TOP2A | 1.88E−264 | DCX | 0 | IGFBP6 | 8.69E−185 |
| IGFBP6 | 0 | KIAA0101 | 9.88E−260 | BASP1 | 0 | ANXA1 | 2.78E−163 |
| RPLP1 | 0 | CKS2 | 1.00E−259 | TUBB2B | 0 | ANXA2 | 1.13E−160 |
| IGFBP5 | 0 | NUSAP1 | 3.17E−248 | TRH | 0 | RPS18 | 2.19E−150 |
| RPL10 | 0 | AURKB | 2.42E−242 | CRMP1 | 0 | CXCL14 | 1.01E−147 |
| IGF2 | 0 | SMC4 | 7.31E−242 | PPP1R1A | 0 | RPS6 | 2.20E−143 |
| MFAP4 | 0 | CDK1 | 4.67E−241 | STMN1 | 0 | RPL12 | 3.64E−138 |
| DCN | 0 | CDKN3 | 2.62E−226 | ETFB | 0 | S100A16 | 3.63E−132 |
| MMP2 | 0 | TYMS | 2.22E−223 | MALAT1 | 0 | FXYD3 | 6.65E−132 |
| FOXL2 | 0 | UBE2S | 4.95E−223 | CKB | 0 | RAB25 | 1.32E−121 |
| RPL13A | 0 | MAD2L1 | 3.78E−215 | RAB3A | 0 | SAT1 | 7.84E−115 |
| TCF4 | 0 | UBE2T | 8.57E−211 | SNCG | 0 | RPL27A | 3.72E−114 |
| KCNQ1OT1 | 0 | KPNA2 | 1.90E−205 | SCG3 | 0 | RPL35 | 1.58E−111 |
| RPL27A | 0 | CCNA2 | 6.60E−203 | HN1 | 0 | ARPC2 | 8.16E−110 |
| CRABP2 | 0 | CENPF | 3.39E−194 | ELAVL3 | 0 | RPL41 | 2.02E−109 |
| TWIST1 | 0 | CCNB1 | 5.36E−193 | ENO2 | 0 | RPL29 | 2.25E−105 |
| MFAP2 | 0 | CCNB2 | 1.58E−188 | CD24 | 0 | RPLP1 | 1.43E−98 |
| PLAC9 | 0 | CENPW | 4.72E−184 | SOX11 | 0 | RPL34 | 7.41E−97 |
| NFIB | 0 | TUBB4B | 1.54E−183 | RORB | 0 | GAPDH | 2.69E−96 |

| Cluster 16 | Cluster 16 | Cluster 17 | Cluster 17 | Cluster 18 | Cluster 18 | Cluster 19 | Cluster 19 |
|---|---|---|---|---|---|---|---|
| Gene | P-value | Gene | P-value | Gene | P-value | Gene | P-value |
| TUBA1A | 0 | SFRP2 | 0 | PMEL | 0 | ERICH5 | 0 |
| STMN2 | 0 | GINS2 | 1.88E−281 | DCT | 0 | ALDH1A1 | 0 |
| TUBB2B | 0 | PCNA | 2.81E−270 | SERPINF1 | 0 | DCBLD2 | 0 |
| TUBB | 5.11E−157 | DAPL1 | 1.20E−264 | APOE | 0 | NKAIN4 | 2.01E−295 |
| MLLT11 | 3.56E−149 | DEK | 4.50E−250 | TMEM98 | 0 | FLRT3 | 1.41E−289 |
| TMSB10 | 6.07E−136 | PTH2 | 2.33E−223 | IGFBP7 | 0 | TKT | 3.99E−282 |
| STMN4 | 1.89E−135 | CYP1B1 | 6.89E−217 | ELN | 0 | APLP2 | 7.30E−279 |
| RTN1 | 1.03E−124 | CRABP1 | 1.24E−216 | TRPM3 | 0 | PAX6 | 3.06E−276 |
| TUBB2A | 4.22E−118 | NASP | 5.96E−213 | TYRP1 | 0 | PTGDS | 9.54E−275 |
| STMN1 | 1.17E−114 | MCM7 | 2.47E−210 | SLC2A1 | 0 | PSAT1 | 1.52E−271 |
| HN1 | 1.19E−105 | CDT1 | 1.73E−203 | MITF | 0 | PCDH9 | 1.19E−269 |
| GAP43 | 3.26E−99 | TYMS | 1.26E−201 | TRPM1 | 0 | SLC2A1 | 5.34E−269 |
| BASP1 | 4.27E−90 | RAX | 8.66E−198 | ATP1B1 | 0 | RPS4X | 1.91E−234 |
| CALM2 | 1.77E−82 | SIX6 | 2.28E−196 | NPC2 | 0 | MAB21L1 | 2.55E−220 |
| MARCKSL1 | 3.50E−78 | MCM3 | 7.50E−195 | GJA1 | 0 | LINC01474 | 4.28E−219 |
| MAP1B | 6.17E−66 | H2AFZ | 3.33E−194 | IGFBP5 | 0 | CP | 9.14E−215 |
| SOX4 | 9.60E−66 | COL2A1 | 1.32E−192 | ATP6V1C2 | 0 | RELN | 5.49E−192 |
| BEX1 | 2.27E−60 | DUT | 1.41E−192 | VIM | 0 | TXNIP | 4.58E−180 |
| UCHL1 | 3.60E−58 | HMX1 | 1.25E−186 | HSD17B2 | 9.42E−305 | RPL7 | 1.08E−179 |
| NSG1 | 9.24E−55 | HMGB2 | 1.29E−185 | TSPAN10 | 3.98E−297 | SPOCK1 | 3.98E−177 |
| CRMP1 | 3.10E−53 | KIAA0101 | 6.96E−184 | GSTP1 | 4.24E−287 | SFRP2 | 6.87E−177 |
| RAB3A | 1.80E−52 | NAP1L1 | 5.36E−178 | PTGDS | 7.15E−281 | ARL4A | 1.46E−173 |
| GNG3 | 3.20E−49 | DKK3 | 7.08E−173 | TYR | 7.95E−280 | DAPL1 | 3.42E−169 |
| CCNI | 1.73E−47 | E2F1 | 2.40E−172 | CSTB | 2.14E−259 | RPS12 | 3.43E−169 |
| CD24 | 2.33E−47 | CCND1 | 6.56E−170 | OTX2 | 3.19E−259 | SIX3 | 2.58E−165 |
| DCX | 4.05E−44 | PSIP1 | 1.89E−164 | CTSH | 1.62E−255 | PCP4 | 2.85E−165 |
| PCSK1N | 3.30E−40 | HELLS | 2.46E−161 | ITM2C | 1.93E−246 | RPL5 | 1.11E−162 |
| HMP19 | 5.27E−38 | CENPH | 4.52E−161 | TMEM176A | 4.19E−238 | HSD17B2 | 6.04E−162 |
| CFL1 | 1.10E−37 | TUBA1B | 3.30E−159 | MT-ND4 | 4.77E−236 | PDGFRA | 1.76E−159 |
| H3F3B | 1.74E−34 | FEN1 | 4.13E−154 | TIMP3 | 1.72E−228 | TSC22D1 | 3.55E−154 |

| Cluster 20 | Cluster 20 | Cluster 21 | Cluster 21 | Cluster 22 | Cluster 22 | Cluster 23 | Cluster 23 |
|---|---|---|---|---|---|---|---|
| Gene | P-value | Gene | P-value | Gene | P-value | Gene | P-value |
| DDIT3 | 3.21E−254 | TPPP3 | 0 | S100A14 | 0 | MT1X | 0 |
| TCEA1 | 2.19E−231 | SFRP2 | 1.33E−202 | KRT17 | 0 | MT2A | 0 |
| HMGA1 | 6.97E−231 | HMX1 | 3.81E−190 | KRT19 | 0 | MT1E | 8.96E−263 |
| HSPA9 | 3.79E−211 | UBB | 5.80E−183 | PERP | 0 | MT1F | 2.65E−239 |
| GDF15 | 1.08E−188 | H3F3A | 4.09E−177 | IGFBP6 | 0 | MT1G | 6.85E−114 |
| SLC3A2 | 3.26E−182 | DAPL1 | 1.77E−172 | S100A11 | 0 | ENO1 | 1.68E−52 |
| BBC3 | 7.93E−179 | DYNLL1 | 8.09E−170 | HSPB1 | 0 | RPL39 | 1.15E−47 |
| DDIT4 | 1.02E−165 | TMSB15A | 6.10E−165 | PDLIM1 | 0 | RPL37A | 4.51E−46 |
| APOE | 6.32E−158 | MEST | 8.43E−163 | WNT6 | 0 | RPS29 | 1.23E−44 |
| POU5F1 | 9.46E−157 | CKB | 1.82E−161 | AQP3 | 0 | RPS27 | 2.86E−41 |
| PMAIP1 | 2.08E−153 | HNRNPA1 | 3.72E−158 | S100A10 | 0 | RPL36 | 8.97E−41 |
| SQSTM1 | 3.72E−150 | PTH2 | 8.61E−153 | KRT8 | 0 | PTH2 | 1.29E−38 |
| UPP1 | 4.18E−149 | CRABP1 | 1.79E−151 | FXYD3 | 0 | DAPL1 | 1.66E−37 |
| MTHFD2 | 2.12E−141 | HNRNPA2B1 | 7.40E−151 | CLDN6 | 0 | RPL38 | 6.70E−36 |
| PLA2G16 | 4.68E−135 | SIX6 | 2.56E−147 | S100A16 | 0 | RPS21 | 3.96E−35 |
| EPCAM | 1.43E−133 | CCNA1 | 1.12E−142 | ARPC2 | 0 | BNIP3 | 6.25E−30 |
| EIF2S2 | 1.60E−112 | CYP1B1 | 2.69E−138 | KRT15 | 0 | RPL34 | 8.16E−30 |
| HSPD1 | 4.42E−111 | TECR | 7.71E−137 | ANXA1 | 0 | RPS15 | 3.60E−28 |
| FTL | 2.11E−110 | PTMS | 1.55E−136 | CLDN4 | 0 | PGK1 | 6.95E−28 |
| AARS | 1.90E−106 | RAX | 9.68E−136 | RAB25 | 0 | NR2F1 | 7.50E−26 |
| HSPA5 | 1.37E−105 | HMGN3 | 2.73E−135 | BCAM | 0 | MIF | 8.53E−26 |
| SARS | 1.98E−103 | ALDH1A1 | 6.79E−134 | HOTAIRM1 | 0 | ALDOA | 2.36E−25 |
| ATF4 | 4.89E−100 | TEKT2 | 4.76E−125 | SFN | 0 | SOX2 | 5.35E−25 |
| PRDX1 | 3.32E−99 | METRN | 9.18E−124 | SPINT1 | 0 | NUDT4 | 2.46E−24 |
| BBS9 | 2.81E−95 | WDR54 | 8.42E−121 | KRT18 | 0.00E+00 | CKB | 4.55E−23 |
| HSP90AA1 | 3.90E−95 | RBMX | 8.38E−118 | SVIL | 6.56E−303 | RPS7 | 4.57E−22 |
| MID1IP1 | 3.26E−93 | CCDC34 | 4.09E−115 | SNCG | 9.02E−292 | RPS15A | 1.53E−21 |
| ATF3 | 2.17E−90 | PTMA | 5.28E−115 | SPINT2 | 1.36E−291 | RPL35A | 3.86E−20 |
| GARS | 1.34E−86 | PTTG1 | 1.85E−114 | TMEM40 | 2.06E−291 | COL2A1 | 4.68E−20 |
| YARS | 4.07E−86 | PEBP1 | 1.51E−112 | EPCAM | 5.76E−285 | RPL7 | 6.30E−20 |

| Cluster 24 | Cluster 24 | Cluster 25 | Cluster 25 | Cluster 26 | Cluster 26 |
|---|---|---|---|---|---|
| Gene | P-value | Gene | P-value | Gene | P-value |
| KRT18 | 0 | PCBP4 | 0 | TTR | 1.87E−199 |
| CLDN4 | 0 | FAM57B | 0 | WLS | 2.66E−158 |
| KRT8 | 0 | NEUROD1 | 0 | CDO1 | 1.23E−148 |
| GABRP | 0 | PDC | 0 | RSPO2 | 1.04E−145 |
| ANXA1 | 0 | 4-Sep | 0 | TPBG | 1.30E−136 |
| KRT19 | 0 | C8orf46 | 0 | CNTNAP2 | 1.92E−135 |
| CLDN7 | 0 | ENO2 | 7.66E−305 | PCP4 | 2.04E−130 |
| S100A11 | 0 | GADD45G | 5.51E−303 | MEST | 3.07E−117 |
| RAB25 | 0 | GNB3 | 1.85E−297 | LYPD1 | 5.34E−106 |
| GPRC5A | 0 | OTX2 | 1.41E−294 | CA2 | 3.57E−105 |
| SPINT1 | 0 | MIR7-3HG | 3.42E−274 | CXCL14 | 5.28E−102 |
| RHOV | 0 | TUBB4B | 6.87E−274 | CYSTM1 | 1.80E−93 |
| S100A10 | 0 | STMN2 | 4.08E−268 | BNIP3 | 1.10E−89 |
| VAMP8 | 0 | SSTR2 | 7.86E−248 | OTX2 | 3.17E−82 |
| ARHGAP29 | 0 | MAP1B | 1.11E−244 | GPC3 | 6.17E−82 |
| CD9 | 0 | THRB | 4.88E−243 | MPC1 | 1.39E−81 |
| ANXA2 | 0 | HES6 | 3.69E−240 | IGFBP2 | 1.04E−80 |
| PERP | 0 | RCVRN | 7.44E−234 | SDC2 | 9.37E−79 |
| CD24 | 0 | GUK1 | 1.28E−233 | RHBDD2 | 6.28E−72 |
| MAL2 | 0 | TPI1 | 6.14E−229 | EMC2 | 5.45E−71 |
| EPCAM | 0 | C11orf96 | 1.14E−226 | SOX2 | 5.90E−71 |
| ATF3 | 0 | H3F3B | 1.18E−226 | MLF1 | 6.49E−70 |
| ELF3 | 0 | DLL3 | 1.18E−225 | PIFO | 2.09E−69 |
| SAT1 | 0 | NEUROD4 | 3.44E−224 | IFI27L1 | 5.87E−69 |
| KLF6 | 0 | CKB | 7.38E−210 | ALDOA | 1.57E−66 |
| ETHE1 | 0 | SCG3 | 7.91E−209 | HSBP1 | 6.25E−66 |
| HSPB1 | 0 | DCT | 1.03E−204 | SEPW1 | 7.56E−66 |
| LGALS3 | 0 | SPCS1 | 5.49E−204 | NRN1 | 2.75E−65 |
| TNFRSF12A | 0 | BSG | 1.03E−203 | LHFP | 6.11E−64 |
| LSR | 0 | NRN1 | 2.62E−202 | GGH | 1.23E−62 |

| Cluster 27 | Cluster 27 | Cluster 28 | Cluster 28 | Cluster 29 | Cluster 29 |
|---|---|---|---|---|---|
| Gene | P-value | Gene | P-value | Gene | P-value |
| IGF2 | 1.70E−235 | FABP7 | 2.38E−258 | MAP1B | 1.11E−243 |
| SPARC | 1.79E−224 | C1orf61 | 2.40E−256 | MLLT11 | 1.39E−241 |
| DLK1 | 1.71E−223 | LINC01551 | 3.00E−225 | TUBA1A | 1.86E−240 |
| COL3A1 | 8.74E−212 | SOX2 | 4.25E−173 | TUBB2B | 7.99E−237 |
| IGFBP3 | 1.09E−211 | CKB | 7.91E−172 | BASP1 | 6.75E−235 |
| DCN | 2.08E−199 | TTYH1 | 1.83E−170 | STMN2 | 3.81E−227 |
| IFITM3 | 1.77E−196 | ID4 | 6.45E−166 | HN1 | 9.57E−227 |
| COL1A1 | 3.72E−192 | VIM | 2.87E−143 | DCX | 2.03E−220 |
| COL6A2 | 3.87E−183 | GPM6B | 3.86E−120 | RTN1 | 1.17E−219 |
| LUM | 3.72E−172 | HES5 | 7.14E−117 | RAB3A | 6.71E−215 |
| FMOD | 5.93E−157 | METRN | 2.28E−116 | CRMP1 | 5.04E−213 |
| FN1 | 5.44E−152 | HOPX | 6.09E−110 | TUBB2A | 1.59E−211 |
| TIMP1 | 7.14E−152 | PTN | 6.77E−105 | STMN4 | 8.64E−201 |
| TGFBI | 3.49E−144 | YBX1 | 3.17E−103 | GAP43 | 4.17E−196 |
| COL5A1 | 1.49E−143 | DOK5 | 2.77E−101 | CNTNAP2 | 3.28E−187 |
| COL1A2 | 1.18E−141 | IGFBP2 | 1.18E−98 | TUBB | 8.48E−184 |
| COL6A1 | 5.44E−138 | TUBB2B | 4.53E−98 | OLFM1 | 1.86E−176 |
| KDELR2 | 7.74E−137 | TMEM161B- | 7.28E−96 | BEX1 | 2.70E−173 |
| | | AS1 | | | |
| PITX1 | 6.70E−135 | GPC3 | 1.69E−88 | MARCKSL1 | 2.96E−172 |
| SERPINH1 | 3.58E−134 | MAP1B | 4.39E−77 | STMN1 | 2.29E−169 |
| BST2 | 2.61E−121 | FZD3 | 4.10E−75 | SOX4 | 1.14E−162 |
| MFAP4 | 1.16E−120 | SYNE2 | 2.40E−73 | KIF5C | 9.67E−161 |
| LMCD1 | 1.55E−119 | TUBA1A | 4.94E−73 | ELAVL4 | 9.70E−161 |
| COL6A3 | 5.79E−119 | SOX3 | 5.39E−66 | HMP19 | 1.42E−155 |
| FTL | 2.79E−113 | SMS | 1.77E−65 | UCHL1 | 1.56E−155 |
| S100A10 | 2.74E−112 | FABP5 | 6.06E−65 | NSG1 | 3.74E−154 |
| COL15A1 | 1.30E−111 | SRI | 2.99E−63 | TERF2IP | 4.55E−152 |
| FXYD5 | 3.36E−110 | PTPRZ1 | 3.52E−62 | CAMK2N1 | 8.96E−152 |
| FKBP10 | 1.24E−108 | FXYD6 | 1.91E−61 | TAGLN3 | 9.34E−149 |
| LGALS1 | 8.71E−103 | FOXG1 | 9.53E−61 | ENO2 | 2.82E−148 |

| Cluster 30 | Cluster 30 | Cluster 31 | Cluster 31 | Cluster 32 | Cluster 32 |
|---|---|---|---|---|---|
| Gene | P-value | Gene | P-value | Gene | P-value |
| HMX1 | 1.16E−97 | CRYAB | 1.96E−110 | PTPRS | 1.50E−13 |
| PTH2 | 1.94E−90 | CRYBB3 | 3.61E−101 | RPL7 | 6.55E−12 |
| RAX | 4.60E−85 | LIM2 | 6.45E−97 | RPL21 | 8.75E−10 |
| COL2A1 | 1.06E−83 | CRYBB2 | 7.37E−91 | RPS15 | 2.20E−09 |
| RPS7 | 1.23E−78 | S100A4 | 1.44E−62 | RPS2 | 3.76E−09 |
| DAPL1 | 1.43E−76 | TRPM3 | 2.08E−60 | SOX11 | 4.70E−09 |
| TRH | 7.55E−76 | CRYBB1 | 2.31E−59 | RPL11 | 5.77E−09 |
| CYP1B1 | 9.96E−74 | CRYBA1 | 4.41E−59 | CTNNB1 | 7.17E−09 |
| MSI2 | 1.17E−73 | PROX1 | 2.32E−54 | RPS3A | 7.78E−09 |
| NUDT4 | 4.49E−71 | TP53I3 | 3.31E−52 | RPS15A | 8.21E−09 |
| RPL7 | 1.96E−67 | PITX3 | 4.02E−48 | LRRC75A | 1.20E−08 |
| MIAT | 4.35E−66 | FABP5 | 2.11E−47 | RPL34 | 1.62E−08 |
| RPL39 | 4.82E−66 | SERPINB6 | 1.49E−46 | RPS19 | 2.60E−08 |
| SFRP2 | 2.94E−65 | CRYBA4 | 1.04E−44 | RPL35A | 5.60E−08 |
| RPL30 | 4.11E−64 | CRYGS | 3.18E−43 | RPS7 | 6.28E−08 |
| LHX2 | 4.19E−63 | CRYGC | 2.66E−42 | SET | 2.85E−07 |
| RPS17 | 9.06E−63 | CP | 1.09E−41 | CYP1B1 | 4.27E−07 |
| SIX6 | 1.63E−62 | IGFBP7 | 2.01E−41 | RPS3 | 4.94E−07 |
| RPS24 | 2.42E−60 | P3H2 | 7.27E−41 | NAP1L1 | 1.09E−06 |
| RAB31 | 8.85E−59 | ALDH1A1 | 1.16E−39 | RPLP0 | 2.09E−06 |
| RPS3 | 4.28E−58 | FOXE3 | 1.30E−38 | RPL39 | 3.80E−06 |
| RDH10 | 6.13E−58 | GJA3 | 3.54E−37 | RPS8 | 7.96E−06 |
| NAP1L1 | 1.52E−56 | SSX2IP | 1.13E−36 | C1orf56 | 8.80E−06 |
| NR2F1 | 8.34E−56 | MAF | 8.46E−35 | RPS13 | 1.10E−05 |
| RPL18 | 4.80E−54 | BHMT | 1.31E−34 | PHKG1 | 1.41E−05 |
| FGF19 | 1.12E−53 | LYPD6B | 7.84E−33 | RPS27 | 1.51E−05 |
| RPLP2 | 7.74E−53 | GJA8 | 3.65E−32 | RPL13 | 1.83E−05 |
| ZFP36L2 | 1.05E−52 | MRPS6 | 7.26E−32 | RPS24 | 2.21E−05 |
| SPP1 | 1.21E−52 | SUCO | 2.77E−31 | RPL31 | 3.34E−05 |
| RPL9 | 3.29E−52 | RP11- | 1.45E−30 | RPS14 | 3.50E−05 |
| | | 490M8.1 | | | |

Table 6B

| Cluster 0 | Cluster 0 | Cluster 1 | Cluster 1 | Cluster 2 | Cluster 2 | Cluster 3 | Cluster 3 |
|---|---|---|---|---|---|---|---|
| Gene | P-value | Gene | P-value | Gene | P-value | Gene | P-value |
| PTMA | 0 | TPM2 | 0 | WLS | 0 | ANKRD1 | 0 |
| RPS2 | 0 | MYL6 | 0 | CRABP1 | 0 | TPM1 | 0 |
| TMSB15A | 0 | TMSB4X | 0 | COL3A1 | 0 | GSTP1 | 8.28E−290 |
| RPL41 | 0 | FTL | 0 | CDH6 | 0 | TMSB4X | 1.34E−287 |
| RPLP0 | 0 | TAGLN | 0 | PRSS23 | 0 | RPL37A | 6.94E−259 |
| YBX1 | 0 | CRABP1 | 0 | SEMA3A | 0 | RPLP2 | 4.65E−253 |
| RPS19 | 0 | MYL9 | 0 | AKAP12 | 0 | RPLP1 | 9.12E−229 |
| HMGN2 | 0 | S100A10 | 0 | SPARC | 0 | RPS20 | 1.70E−225 |
| GAPDH | 0 | TMSB10 | 0 | IGFBP2 | 0 | TAGLN | 1.05E−219 |
| RPL23A | 0 | VIM | 0 | GPM6B | 0 | RPS19 | 2.07E−212 |
| H3F3A | 0 | ANXA1 | 0 | COL1A2 | 0 | RPS10 | 2.97E−208 |
| RPSA | 0 | ANXA2 | 1.11E−288 | MT-ND3 | 0 | RPL27A | 9.98E−203 |
| RPS24 | 0 | CALD1 | 2.92E−288 | MT-CO1 | 0 | RPL23 | 4.39E−186 |
| RPS27L | 0 | S100A11 | 1.55E−243 | PTX3 | 0 | NPPB | 1.87E−185 |
| RPLP1 | 0 | ACTB | 1.74E−226 | DST | 0 | RPL35 | 4.91E−170 |
| LINC01551 | 0 | RPS18 | 1.17E−209 | ITGB1 | 0 | RPS26 | 1.01E−160 |
| RPS25 | 0 | RPL35 | 1.45E−207 | MT-CYB | 0 | RPL23A | 3.50E−134 |
| RANBP1 | 0 | GNG11 | 3.43E−200 | HSPA5 | 0 | TMSB10 | 5.63E−133 |
| HES5 | 0 | RPS12 | 8.27E−199 | PCDH9 | 0 | RPL36A | 2.02E−132 |
| RPL18A | 0 | PFN1 | 1.21E−182 | SPOCK1 | 0 | RPS18 | 2.25E−132 |
| RPL35 | 0 | CFL1 | 5.55E−171 | NES | 0 | RPS2 | 1.35E−129 |
| HMGA1 | 0 | GNG5 | 1.76E−156 | SDC2 | 0 | ACTG1 | 2.84E−127 |
| RPS15 | 0 | RPL10 | 1.22E−151 | VIM | 0 | RPLP0 | 2.77E−125 |
| RPS13 | 0 | EIF1 | 4.41E−147 | PEG10 | 0 | RPS27A | 1.72E−119 |
| RPL21 | 1.42E−307 | RPS26 | 2.71E−135 | HSP90B1 | 0 | RPS12 | 6.50E−119 |
| RPS10 | 6.27E−302 | RPL32 | 8.19E−134 | MT-CO2 | 0 | RPL41 | 2.59E−113 |
| RPS8 | 1.68E−297 | RPL7 | 1.01E−130 | MT-CO3 | 0 | RPL26 | 3.35E−110 |
| HNRNPA1 | 5.54E−294 | PRDX6 | 1.39E−129 | PLD3 | 0 | UBA52 | 6.45E−109 |
| MARCKSL1 | 9.23E−291 | SPATS2L | 4.41E−122 | MAP2 | 0 | RPL21 | 9.69E−108 |
| RPLP2 | 2.26E−278 | DSTN | 2.18E−121 | CCND2 | 0 | ANXA1 | 1.06E−106 |

| Cluster 4 | Cluster 4 | Cluster 5 | Cluster 5 | Cluster 6 | Cluster 6 |
|---|---|---|---|---|---|
| Gene | P-value | Gene | P-value | Gene | P-value |
| RPL41 | 0 | NAV1 | 0 | CENPF | 0 |
| GSTP1 | 0 | SFRP2 | 0 | CCNB1 | 0 |
| RPL13A | 0 | EMX2 | 0 | UBE2S | 0 |
| RPS2 | 2.42E−300 | GJA1 | 0 | TPX2 | 0 |
| RPS20 | 7.92E−298 | TPBG | 0 | ARL6IP1 | 0 |
| RPL21 | 6.43E−279 | CDH2 | 0 | CENPE | 0 |
| CNTNAP2 | 4.57E−272 | ARL4C | 0 | TUBB4B | 0 |
| TMSB15A | 5.30E−269 | GPC3 | 0 | CDC20 | 0 |
| RPLP0 | 1.45E−247 | PAX6 | 0 | DLGAP5 | 0 |
| RPL27A | 4.33E−239 | MT-ND2 | 0 | CRABP1 | 0 |
| RPLP2 | 4.29E−226 | MT-ND1 | 0 | CCNB2 | 0 |
| RPLP1 | 3.94E−207 | SYNE2 | 0 | TOP2A | 0 |
| RPS8 | 5.72E−204 | GLI3 | 0 | ASPM | 0 |
| RPS27 | 1.31E−200 | CLU | 0 | UBE2C | 0 |
| RPL12 | 2.05E−195 | ATP2B1 | 0 | PTTG1 | 0 |
| RPL31 | 1.40E−193 | TRIM24 | 0 | CKAP2 | 0 |
| RPS15 | 1.21E−169 | ZIC1 | 0 | HMMR | 0 |
| RPL37A | 1.46E−168 | CNTNAP2 | 0 | SGO2 | 0 |
| RPL35 | 1.94E−152 | FZD3 | 0 | KPNA2 | 0 |
| SNRPD2 | 2.22E−146 | AKAP9 | 0 | AURKA | 0 |
| YBX1 | 6.81E−145 | GABPB1- | 0 | GNG11 | 0 |
| | | AS1 | | | |
| RPL34 | 1.45E−143 | MT-ND6 | 0 | PRSS23 | 0 |
| RPS19 | 5.29E−140 | MYO10 | 0 | NR2F2 | 0 |
| TMA7 | 8.83E−140 | TLE4 | 0 | PLK1 | 0 |
| RPS11 | 1.78E−135 | CENPF | 0 | CALM2 | 0 |
| RPS14 | 6.24E−132 | MFAP2 | 0 | CDH6 | 0 |
| RPS28 | 1.06E−126 | TPR | 0 | MKI67 | 0 |
| RPL9 | 1.82E−126 | KCNQ1OT1 | 0 | PTX3 | 0 |
| RPL7 | 3.14E−126 | CCND1 | 0 | LYPD1 | 0 |
| HES4 | 1.50E−122 | FADS1 | 0 | HSP90B1 | 0 |

| Cluster 7 | Cluster 7 | Cluster 8 | Cluster 8 | Cluster 9 | Cluster 9 |
|---|---|---|---|---|---|
| Gene | P-value | Gene | P-value | Gene | P-value |
| DLK1 | 0 | CRABP1 | 0 | EEF1A1 | 0 |
| MT-CO3 | 0 | WLS | 0 | HNRNPA1 | 0 |
| FOXG1 | 0 | HIST1H4C | 0 | RPS10 | 8.73E−246 |
| SYNE2 | 0 | PTN | 0 | RPL36A | 9.40E−180 |
| LINC01551 | 0 | COL3A1 | 0 | TMSB15A | 1.99E−158 |
| SFRP1 | 0 | MT-ND3 | 0 | DLK1 | 1.84E−156 |
| LHX2 | 0 | PTX3 | 0 | LINC01551 | 4.55E−141 |
| MT-CO2 | 0 | ALKAL2 | 0 | RPS24 | 1.21E−106 |
| GLI3 | 0 | HIST1H1E | 0 | RPS3 | 5.20E−102 |
| IGDCC3 | 0 | PRSS23 | 0 | RPS3A | 6.44E−96 |
| TRIM24 | 0 | CDH6 | 0 | GAPDH | 8.74E−94 |
| HES5 | 0 | SEMA3A | 0 | RPL6 | 5.92E−92 |
| ID4 | 0 | MT-CO1 | 0 | PTMS | 2.21E−82 |
| MEIS2 | 0 | AKAP12 | 0 | RPL13A | 7.53E−81 |
| PTPRZ1 | 0 | PEG10 | 0 | MARCKSL1 | 9.66E−75 |
| VCAN | 0 | SPOCK1 | 0 | RPL3 | 2.23E−72 |
| MT-ND1 | 0 | HIST1H1C | 0 | RPL26 | 7.38E−72 |
| MT-ND4 | 0 | GPM6B | 0 | RPL37A | 5.73E−69 |
| LIX1 | 0 | SPARC | 0 | SRP14 | 1.92E−67 |
| MYCN | 0 | MT-CYB | 0 | RPS11 | 4.38E−66 |
| EMX2 | 0 | HIST1H1B | 0 | RPL23A | 6.79E−66 |
| MT-CYB | 0 | MT-ND4 | 0 | RPL7A | 1.25E−65 |
| PRKDC | 0 | CALU | 0 | TUBA1A | 2.95E−60 |
| PTBP2 | 0 | ITGB1 | 0 | RPL41 | 4.81E−59 |
| SIX3 | 0 | DST | 0 | TPT1 | 1.06E−57 |
| CHD7 | 0 | HIST1H1D | 0 | RPS4X | 1.23E−57 |
| EPB41 | 0 | COL1A2 | 0 | NNAT | 1.48E−55 |
| MT-ND3 | 0 | RHOB | 0 | STMN1 | 4.14E−54 |
| PTMA | 0 | GNG11 | 0 | PRDX2 | 7.57E−53 |
| SOX3 | 0 | CDK1 | 0 | RPS27 | 1.26E−52 |

| Cluster 10 | Cluster 10 | Cluster 11 | Cluster 11 | Cluster 12 | Cluster 12 |
|---|---|---|---|---|---|
| Gene | P-value | Gene | P-value | Gene | P-value |
| S100A11 | 0 | NNAT | 0 | PLCG2 | 1.34E−43 |
| MYL12A | 0 | CDKN1C | 0 | TLE4 | 1.72E−27 |
| TAGLN | 0 | SOX4 | 0 | MALAT1 | 1.72E−26 |
| CTGF | 0 | TUBA1A | 0 | RPS15 | 5.35E−24 |
| TPM1 | 0 | TAGLN3 | 0 | HES1 | 7.46E−24 |
| CALD1 | 0 | STMN1 | 0 | RPL36 | 6.96E−21 |
| TMSB10 | 0 | MLLT11 | 0 | RPS19 | 3.57E−20 |
| FN1 | 0 | STMN2 | 0 | RPS27 | 4.83E−19 |
| COL8A1 | 0 | HES6 | 0 | CDKN1C | 2.24E−18 |
| TPM4 | 0 | CKB | 0.00E+00 | SOX4 | 2.89E−18 |
| ACTG2 | 0 | TUBB2B | 6.71E−297 | RPL28 | 4.70E−13 |
| COL4A1 | 0 | H3F3A | 8.43E−297 | PIK3R3 | 6.10E−13 |
| KRT18 | 0 | MAP1B | 3.48E−273 | RPL13 | 8.48E−13 |
| COL1A2 | 0 | SOX11 | 1.26E−269 | BTG1 | 2.24E−12 |
| ITGA1 | 0 | MAP6 | 2.58E−247 | RPL35 | 4.81E−12 |
| DSTN | 0 | BASP1 | 4.65E−232 | MTRNR2L12 | 2.33E−10 |
| COL1A1 | 0 | DCX | 2.55E−230 | RPL37A | 2.64E−10 |
| PHLDA2 | 0 | PCBP4 | 7.01E−227 | DLK1 | 4.21E−10 |
| FSTL1 | 0 | ELAVL4 | 1.39E−220 | IER5 | 1.43E−09 |
| BGN | 0 | ELAVL2 | 4.10E−201 | RPL13A | 1.70E−09 |
| SEPT11 | 0 | ELAVL3 | 1.38E−198 | AC025164.1 | 1.74E−09 |
| TPM2 | 0 | RBP1 | 3.78E−197 | AL021453.1 | 1.03E−08 |
| ITGB1 | 0 | CD24 | 4.44E−183 | RPS27L | 1.09E−08 |
| COL5A2 | 0 | MIAT | 7.13E−178 | HEXIM1 | 1.78E−08 |
| IGFBP5 | 0 | INSM1 | 9.74E−178 | HIST1H2AG | 2.87E−08 |
| COL4A2 | 0 | NREP | 4.62E−177 | RPL38 | 2.94E−08 |
| IGFBP7 | 0 | ATP6V1G1 | 1.34E−166 | RPL37 | 3.52E−08 |
| TGFB2 | 0 | DLL3 | 2.87E−164 | AMD1 | 8.52E−08 |
| ACTN1 | 0 | DPYSL3 | 3.35E−158 | MAFB | 1.94E−07 |
| RHOBTB3 | 0 | KIF5C | 1.73E−146 | RPS21 | 2.21E−07 |

Table 6C

| Cluster 0 | Cluster 0 | Cluster 1 | Cluster 1 | Cluster 2 | Cluster 2 | Cluster 3 | Cluster 3 |
|---|---|---|---|---|---|---|---|
| Gene | P-value | Gene | P-value | Gene | P-value | Gene | P-value |
| STMN2 | 0 | TUBA1A | 2.01E−251 | FGFBP3 | 0 | TSPAN13 | 0 |
| NNAT | 0 | TUBB | 1.64E−244 | VIM | 0 | MDH1 | 0 |
| GPM6A | 0 | STMN2 | 1.25E−240 | NTRK2 | 0 | ARF4 | 0 |
| STMN1 | 0 | SOX4 | 5.77E−235 | HES1 | 0 | ATP6V0B | 0 |
| CELF4 | 0 | MLLT11 | 8.39E−234 | TTYH1 | 0 | RABAC1 | 0 |
| TUBB2B | 0 | STMN1 | 1.42E−220 | SCD | 0 | CUTA | 0 |
| TUBB | 0 | STMN4 | 2.68E−217 | ZFP36L1 | 0 | COPE | 0 |
| BASP1 | 0 | MALAT1 | 3.02E−212 | SOX2 | 0 | COX8A | 0 |
| H3F3B | 0 | TUBB2B | 3.73E−209 | SPARC | 0 | SERF2 | 0 |
| TUBB2A | 0 | GPM6A | 3.40E−150 | CD99 | 0 | ATP5F1B | 0 |
| TUBA1A | 0 | PTMS | 1.05E−137 | NPC2 | 0 | VAMP2 | 0 |
| STMN4 | 0 | CCNI | 2.01E−133 | CDKN1A | 0 | PTPRN | 0 |
| RTN1 | 0 | GAP43 | 2.57E−133 | RCN1 | 0 | MAGEH1 | 0 |
| NOVA1 | 0 | TMSB15A | 2.40E−130 | TMBIM6 | 0 | ARPC3 | 0 |
| MARCKSL1 | 0 | PEG10 | 1.38E−121 | GPM6B | 0 | ATP5MC1 | 0 |
| SOX4 | 0.00E+00 | H3F3B | 2.24E−110 | PSAP | 0 | VGF | 0 |
| SEZ6L2 | 1.87E−301 | TUBB2A | 4.88E−97 | HSPA5 | 0 | NDUFAF3 | 0 |
| H3F3A | 6.07E−294 | MARCKSL1 | 6.13E−95 | HES4 | 0 | SLC22A17 | 0 |
| MAPT | 1.06E−290 | YWHAH | 1.19E−92 | ID4 | 0 | MT-ND1 | 0 |
| MALAT1 | 2.71E−284 | NNAT | 1.17E−91 | EDNRB | 0 | KDELR2 | 0 |
| TMSB15A | 9.97E−275 | FXYD6 | 2.05E−76 | CALU | 0 | BEX1 | 0 |
| CCNI | 4.03E−268 | NREP | 7.48E−71 | CLU | 0 | APBB3 | 0 |
| YWHAH | 1.63E−266 | ZFAS1 | 4.25E−69 | ID3 | 0 | CPE | 0 |
| TSHZ2 | 3.61E−263 | H3F3A | 3.92E−64 | MSMO1 | 0 | SEC61G | 0 |
| POU2F2 | 1.36E−256 | UCHL1 | 3.94E−64 | CXCR4 | 0 | SCG2 | 0 |
| NSG2 | 5.10E−256 | TMSB10 | 2.57E−62 | MGST1 | 0 | MORF4L2 | 0 |
| GAP43 | 5.45E−252 | RTN1 | 3.60E−59 | NEAT1 | 0 | OCIAD1 | 0 |
| NREP | 3.27E−246 | PTH2 | 9.51E−59 | SAT1 | 0 | PRDX5 | 0 |
| FXYD6 | 6.33E−241 | BASP1 | 1.01E−56 | PDLIM3 | 0 | SELENOM | 0 |
| MLLT11 | 1.46E−240 | RPL34 | 2.65E−56 | RHOC | 0 | ATP6V1G2 | 0 |

| Cluster 4 | Cluster 4 | Cluster 5 | Cluster 5 | Cluster 6 | Cluster 6 | Cluster 7 | Cluster 7 |
|---|---|---|---|---|---|---|---|
| Gene | P-value | Gene | P-value | Gene | P-value | Gene | P-value |
| DLX2 | 0 | EIF1 | 2.28E−190 | SOX4 | 6.41E−134 | RPS19 | 0 |
| DLX6-AS1 | 0 | KRT10 | 4.51E−181 | GPM6A | 1.21E−133 | ID1 | 1.33E−297 |
| DLX5 | 0 | VAMP2 | 1.64E−176 | MARCKSL1 | 5.03E−122 | RPS7 | 9.58E−267 |
| ACTG1 | 0 | ARF1 | 9.63E−139 | MLLT11 | 1.38E−112 | RPL39 | 1.22E−265 |
| RPL34 | 2.45E−301 | ARF4 | 2.64E−134 | NRN1 | 1.98E−112 | RPL13 | 3.94E−259 |
| TUBB | 2.93E−292 | P4HB | 2.88E−132 | STMN2 | 1.19E−103 | CKB | 4.32E−258 |
| SOX4 | 2.43E−283 | GOLGA2 | 1.83E−127 | BASP1 | 1.36E−103 | RPS27L | 1.28E−249 |
| TUBA1A | 1.27E−271 | SEC61G | 4.84E−125 | CD24 | 2.11E−100 | RPLP1 | 2.43E−245 |
| DLX1 | 5.87E−258 | KDELR2 | 5.10E−125 | H3F3A | 9.70E−92 | RPS15A | 2.89E−244 |
| TUBB2B | 4.24E−228 | MVD | 9.68E−122 | TUBA1A | 1.64E−86 | ASCL1 | 1.48E−222 |
| HNRNPA1 | 4.77E−215 | TMED3 | 5.20E−118 | BTG1 | 2.35E−84 | SOX2 | 3.38E−220 |
| RPL39 | 1.67E−205 | CYCS | 1.87E−111 | TUBB2B | 5.43E−76 | HES1 | 9.72E−219 |
| MLLT11 | 4.66E−201 | TRMT112 | 5.65E−111 | MAP1B | 6.07E−72 | RPL7 | 2.58E−217 |
| RPS4X | 3.05E−194 | HERPUD1 | 3.58E−108 | JPT1 | 2.14E−70 | HES6 | 2.06E−216 |
| NNAT | 1.21E−181 | MED10 | 1.42E−104 | CRABP1 | 1.48E−65 | RPS27 | 1.60E−215 |
| PTMS | 2.14E−181 | MORF4L2 | 2.18E−96 | TUBB | 2.31E−64 | RPS12 | 7.83E−213 |
| H3F3A | 1.63E−177 | FDPS | 8.49E−95 | ELAVL4 | 3.04E−64 | RPS15 | 1.35E−209 |
| RPL11 | 2.10E−173 | ATP5ME | 8.92E−94 | LHX5-AS1 | 4.76E−62 | RPS23 | 1.45E−209 |
| RPL7 | 1.77E−163 | SRP54 | 2.19E−92 | PTMA | 1.01E−60 | RPL37 | 2.79E−209 |
| RPL35A | 2.30E−150 | RPL18 | 3.90E−90 | MIAT | 2.67E−59 | RPS6 | 3.15E−205 |
| TMSB10 | 1.61E−148 | NPRL2 | 1.52E−88 | NSG1 | 7.52E−58 | RPL18A | 1.21E−199 |
| MARCKSL1 | 5.88E−146 | MAP1LC3B | 2.05E−87 | STMN1 | 6.87E−52 | CDKN1A | 2.88E−193 |
| RPL6 | 1.55E−140 | LSM3 | 2.31E−83 | CITED2 | 3.86E−51 | RPS9 | 5.23E−189 |
| ZFAS1 | 1.51E−138 | SELENOS | 9.34E−81 | CRNDE | 2.03E−48 | RPS3A | 3.07E−186 |
| RPL24 | 5.99E−137 | UBC | 1.67E−78 | SEZ6L2 | 7.92E−46 | RPL6 | 1.14E−184 |
| GAD2 | 3.26E−135 | MRPL55 | 6.60E−78 | NOVA1 | 1.54E−45 | RPL12 | 5.47E−183 |
| JPT1 | 1.22E−123 | ARPC3 | 6.79E−77 | LHX1 | 2.16E−44 | RPS20 | 6.89E−182 |
| RPL9 | 2.77E−122 | RPS16 | 7.55E−76 | RTN4 | 2.27E−44 | RPS3 | 8.76E−181 |
| COX7C | 6.70E−122 | HMGB2 | 9.53E−75 | ACTG1 | 2.89E−43 | RPS2 | 3.16E−172 |
| TMSB15A | 1.82E−120 | SELENOK | 3.25E−73 | NEFL | 3.62E−43 | RPL36 | 1.97E−170 |

| Cluster 8 | Cluster 8 | Cluster 9 | Cluster 9 | Cluster 10 | Cluster 10 |
|---|---|---|---|---|---|
| Gene | P-value | Gene | P-value | Gene | P-value |
| RPL41 | 0 | COL1A1 | 0 | GATA3 | 4.35E−149 |
| FTL | 0 | COL3A1 | 0 | HOXB5 | 3.05E−135 |
| FTH1 | 0 | COL1A2 | 0 | RBP1 | 1.16E−89 |
| RPS6 | 0 | NUPR1 | 0 | CRABP1 | 3.80E−80 |
| VIM | 0 | PTN | 0 | GATA3-AS1 | 2.69E−69 |
| RPS20 | 0 | IFITM3 | 0 | BTG1 | 2.74E−39 |
| RPLP1 | 8.59E−298 | MGP | 0 | HOXB2 | 1.36E−35 |
| TMSB4X | 1.19E−289 | FN1 | 0 | CD24 | 3.83E−34 |
| RPL36 | 5.98E−287 | RBP1 | 0 | JPT1 | 1.28E−33 |
| RPL12 | 5.47E−286 | S100A11 | 0 | STMN2 | 1.59E−33 |
| RPL35 | 7.09E−281 | RPLP1 | 0 | SOX4 | 1.03E−31 |
| RPL26 | 2.50E−275 | RPS2 | 0 | MARCKSL1 | 6.33E−29 |
| RPS18 | 2.14E−260 | RPS18 | 0 | TUBA1A | 2.28E−28 |
| RPS24 | 2.33E−257 | SLC25A6 | 0 | NR2F1 | 4.38E−28 |
| SERF2 | 1.86E−255 | LGALS1 | 0 | H3F3A | 8.49E−28 |
| RPL27 | 5.35E−255 | SPARC | 0 | HOXB8 | 1.04E−25 |
| RPL31 | 1.28E−254 | IFITM2 | 0 | TERF2IP | 9.63E−25 |
| RPS12 | 7.07E−247 | RPL28 | 0 | MIAT | 9.96E−25 |
| RPS3A | 9.68E−240 | RPS12 | 0 | PAFAH1B3 | 2.65E−24 |
| GSTP1 | 2.90E−236 | RPL23A | 0 | CRNDE | 7.63E−24 |
| NPC2 | 1.98E−229 | RPL13 | 0 | MLLT11 | 1.32E−23 |
| RPS15 | 9.72E−223 | BGN | 0 | NSG1 | 9.49E−23 |
| EEF1A1 | 2.32E−221 | RPS6 | 0 | GATA2 | 3.45E−22 |
| RPS25 | 1.02E−220 | RPL12 | 0 | DDX5 | 2.14E−21 |
| RPL27A | 7.55E−220 | MFAP4 | 0 | NR2F2 | 5.77E−21 |
| RPS10 | 6.57E−219 | SAT1 | 0 | CITED2 | 1.03E−19 |
| RPS15A | 4.07E−217 | ISLR | 0 | TAGLN2 | 1.48E−19 |
| RPS19 | 1.15E−210 | IGFBP5 | 0 | EIF4A2 | 1.80E−19 |
| RPL28 | 2.20E−209 | MDK | 0 | NDUFA4 | 2.57E−19 |
| RPL21 | 1.06E−202 | RPL41 | 0 | GPM6A | 2.69E−19 |

| Cluster 11 | Cluster 11 | Cluster 12 | Cluster 12 | Cluster 13 | Cluster 13 |
|---|---|---|---|---|---|
| Gene | P-value | Gene | P-value | Gene | P-value |
| MT-ND1 | 2.19E−265 | NTRK2 | 6.35E−277 | GNG5 | 6.94E−172 |
| VGF | 1.47E−243 | CLU | 1.32E−271 | VIM | 6.49E−170 |
| COX8A | 5.90E−235 | VIM | 1.90E−267 | CKS1B | 6.04E−150 |
| CUTA | 1.04E−233 | GPM6B | 1.19E−261 | HES1 | 1.12E−147 |
| MT-CYB | 4.68E−233 | EDNRB | 1.16E−232 | ID3 | 2.58E−142 |
| SERF2 | 2.16E−230 | CD99 | 4.87E−221 | RPLP1 | 2.52E−129 |
| TSPAN13 | 6.18E−228 | ACTB | 3.78E−215 | CD99 | 4.85E−125 |
| RABAC1 | 9.05E−225 | ID3 | 4.84E−212 | RPS6 | 2.18E−124 |
| FAIM2 | 4.40E−224 | PDLIM3 | 5.39E−205 | B2M | 4.08E−118 |
| PTPRN | 1.65E−222 | TTYH1 | 4.79E−204 | TUBA1B | 6.01E−113 |
| SNCB | 9.79E−213 | DKK3 | 7.58E−196 | RHOC | 7.39E−112 |
| MT-ND2 | 1.43E−212 | RTN4 | 4.14E−194 | TOP2A | 1.36E−110 |
| PHLDA2 | 1.08E−211 | HSPA8 | 1.12E−191 | PTTG1 | 4.29E−110 |
| ATP6V0B | 2.59E−210 | SERPINE2 | 3.37E−190 | UBE2C | 7.11E−110 |
| MT-ND4 | 3.92E−205 | MGST1 | 3.71E−189 | BIRC5 | 2.12E−109 |
| MDH1 | 1.90E−199 | ID2 | 3.64E−187 | EMP3 | 2.94E−108 |
| ATP5MC1 | 4.45E−194 | UBL3 | 7.47E−187 | NME4 | 4.55E−108 |
| COPE | 6.21E−194 | S100B | 2.15E−186 | GSTP1 | 1.75E−107 |
| SELENOM | 1.32E−190 | CKB | 4.72E−186 | PCLAF | 1.22E−103 |
| LIN7B | 3.69E−190 | NPC2 | 7.99E−185 | CLIC1 | 2.55E−102 |
| EBNA1BP2 | 1.02E−189 | CNN3 | 1.48E−184 | NUSAP1 | 5.00E−100 |
| APBB3 | 3.34E−189 | PFN2 | 4.92E−184 | CKS2 | 6.30E−99 |
| ARPC3 | 2.30E−186 | PSAP | 1.82E−182 | ID1 | 1.05E−98 |
| PRDX5 | 1.02E−185 | SOX2 | 6.49E−180 | TUBB6 | 3.46E−98 |
| NDUFAF3 | 1.02E−180 | ID4 | 2.14E−178 | CNN3 | 7.70E−98 |
| CHMP2B | 4.40E−180 | DDR1 | 1.41E−176 | CDK1 | 8.92E−98 |
| DNAJC3-DT | 7.09E−180 | ENO1 | 1.65E−174 | H2AFV | 2.67E−96 |
| GNG3 | 2.07E−177 | CTGF | 1.35E−169 | HMGB2 | 4.54E−96 |
| ARF4 | 7.34E−175 | B2M | 4.76E−168 | TYMS | 4.40E−94 |
| AL669831.5 | 8.98E−174 | C12orf75 | 5.32E−168 | RPS20 | 1.72E−92 |

| Cluster 14 | Cluster 14 | Cluster 15 | Cluster 15 | Cluster 16 | Cluster 16 |
|---|---|---|---|---|---|
| Gene | P-value | Gene | P-value | Gene | P-value |
| PEG10 | 4.11E−141 | RPL37A | 1.38E−49 | MALAT1 | 8.04E−85 |
| BTG1 | 2.03E−116 | SBK1 | 3.43E−48 | WSB1 | 1.03E−80 |
| TMSB4X | 6.01E−112 | RPS29 | 6.95E−44 | HES1 | 1.02E−37 |
| TUBB2B | 4.90E−71 | CNIH2 | 9.96E−42 | CTGF | 2.52E−31 |
| MLLT11 | 1.28E−48 | POU2F2 | 3.65E−41 | SRSF5 | 6.68E−31 |
| ACTG1 | 1.34E−46 | NUDT3 | 4.06E−41 | VMP1 | 3.36E−30 |
| TUBA1A | 6.95E−38 | DCX | 2.37E−40 | MIR99AHG | 4.68E−28 |
| TUBB | 3.66E−37 | RPL38 | 3.04E−39 | CDK5RAP3 | 8.29E−26 |
| SOX4 | 2.02E−35 | ZFHX3 | 8.09E−39 | DST | 9.09E−24 |
| NKX2-1 | 1.80E−34 | MEX3A | 1.33E−37 | PNISR | 1.40E−22 |
| BRS3 | 2.09E−34 | CAMK2N1 | 3.59E−36 | SORBS2 | 1.22E−20 |
| PTMS | 3.31E−33 | PPP1R1A | 6.71E−36 | ADGRV1 | 4.98E−20 |
| JPT1 | 7.76E−33 | DPYSL3 | 7.94E−36 | SAT2 | 8.65E−20 |
| HTATSF1 | 2.20E−32 | RPS21 | 8.87E−36 | N4BP2L2 | 9.71E−20 |
| H3F3A | 8.21E−30 | RPS27 | 1.06E−35 | ID3 | 3.54E−18 |
| FHL1 | 4.76E−27 | EFNB3 | 3.01E−35 | NOVA1 | 1.68E−16 |
| ETFB | 1.62E−26 | SDC3 | 3.58E−35 | SAT1 | 1.42E−15 |
| STMN4 | 6.88E−26 | NCAM1 | 4.82E−35 | PDGFRB | 1.70E−15 |
| FDFT1 | 8.05E−26 | NACA | 8.88E−35 | ACADVL | 3.90E−15 |
| DLX2 | 1.90E−25 | MAP6 | 4.61E−34 | THUMPD3-AS1 | 5.27E−15 |
| CD24 | 1.02E−24 | RPL30 | 5.06E−34 | NEAT1 | 6.39E−15 |
| MARCKSL1 | 1.83E−24 | SPTAN1 | 5.45E−34 | NTRK2 | 1.85E−13 |
| PCDH17 | 6.82E−23 | SEPT3 | 6.38E−34 | PDLIM3 | 8.97E−13 |
| DCX | 1.70E−22 | DUSP8 | 8.95E−34 | CYR61 | 1.76E−11 |
| NOVA1 | 1.80E−20 | RPL18 | 1.09E−33 | HNRNPDL | 2.10E−11 |
| LHX6 | 1.92E−20 | ATCAY | 2.14E−33 | NAT9 | 2.04E−10 |
| COX7C | 7.48E−20 | AES | 3.23E−33 | PTPRZ1 | 1.01E−09 |
| NREP | 9.12E−20 | RPS11 | 1.41E−32 | RSRP1 | 1.43E−09 |
| MIR7-3HG | 1.42E−19 | EVL | 1.73E−32 | SRSF2 | 9.39E−09 |
| TMSB15A | 1.06E−17 | ZFAS1 | 2.45E−32 | QKI | 1.37E−08 |

TABLE 7
Differentially expressed genes in bulk RNA-seq datasets (see, U.S. Provisional Application 63/219,705 filed
Jul. 8, 2021). (A) For each ORF overexpression condition, genes that were significantly differentially
expressed (t-test q-value < 0.05 with FDR correction) relative to respective GFP overexpressing cells
that were cultured in mTeSR stem cell media are listed with associated fold change and P-values. (B) For
each DYRK1A perturbation, genes that were significantly differentially expressed (t-test q-value <
0.05 with FDR correction) relative to respective controls are listed with associated fold change and P-values.

| Cardiomyocte TFs | | HSC TFs | | Oligodendrocyte TFs | | Hepatocyte TFs | |
|---|---|---|---|---|---|---|---|
| Table 3 Name | nDatasets | Table 3 Name | nDatasets | Table 3 Name | nDatasets | Table 3 Name | nDatasets |
| TFORF2064 | 7 | TFORF3148 | 5 | TFORF0124 | 9 | TFORFO793 | 6 |
| TFORF2065 | 7 | TFORF2242 | 4 | TFORF2534 | 8 | TFORF0691 | 6 |
| TFORF3104 | 7 | TFORF2997 | 4 | TFORF2535 | 8 | TFORF0692 | 6 |
| TFORF2119 | 5 | TFORF2998 | 4 | TFORF2536 | 8 | TFORF0693 | 6 |
| TFORF2120 | 5 | TFORF0974 | 4 | TFORF2095 | 8 | TFORF0694 | 6 |
| TFORF2121 | 5 | TFORF0975 | 4 | TFORF0123 | 6 | TFORF0695 | 6 |
| TFORF1982 | 5 | TFORF0976 | 4 | TFORF2530 | 5 | TFORF2956 | 6 |
| TFORF1983 | 5 | TFORF2823 | 4 | TFORF0094 | 5 | TFORF2957 | 6 |
| TFORF2236 | 4 | TFORF2824 | 4 | TFORF0095 | 5 | TFORF2546 | 5 |
| TFORF2237 | 4 | TFORF2825 | 4 | TFORF0096 | 5 | TFORF0099 | 4 |
| TFORF2238 | 4 | TFORF2826 | 4 | TFORF2497 | 4 | TFORF3398 | 4 |
| TFORF2239 | 4 | TFORF2827 | 4 | TFORF2605 | 4 | TFORFO280 | 4 |
| TFORF0968 | 4 | TFORF3489 | 4 | TFORF2606 | 4 | TFORF2195 | 4 |
| TFORF1182 | 4 | TFORF0943 | 4 | TFORF0110 | 4 | TFORF2196 | 4 |
| TFORF3330 | 4 | TFORF3073 | 4 | TFORF0111 | 4 | TFORF2197 | 4 |
| TFORF1638 | 3 | TFORF3074 | 4 | TFORF3129 | 4 | TFORF2198 | 4 |
| TFORF0969 | 3 | TFORF1497 | 4 | TFORF3237 | 4 | TFORF1638 | 3 |
| TFORF1506 | 3 | TFORF0337 | 4 | TFORF2539 | 4 | TFORF2209 | 3 |
| TFORF3024 | 3 | TFORF0338 | 4 | TFORF2540 | 4 | TFORF2210 | 3 |
| TFORF0480 | 3 | TFORF0339 | 4 | TFORF2541 | 4 | TFORF2211 | 3 |
| TFORF0481 | 3 | TFORF0340 | 4 | TFORF2542 | 4 | TFORF2212 | 3 |
| TFORF3296 | 3 | TFORF0341 | 4 | TFORF2543 | 4 | TFORF1326 | 3 |
| TFORF3297 | 3 | TFORF1938 | 4 | TFORF2938 | 4 | TFORF0981 | 3 |
| TFORF0018 | 3 | TFORF1939 | 4 | TFORF1607 | 3 | TFORF0982 | 3 |
| TFORF0019 | 3 | TFORF0427 | 4 | TFORF1608 | 3 | TFORF0860 | 3 |
| TFORF0282 | 3 | TFORF0428 | 4 | TFORF1885 | 3 | TFORF0861 | 3 |
| TFORF0283 | 3 | TFORF0429 | 4 | TFORF1886 | 3 | TFORF0862 | 3 |
| TFORF0284 | 3 | TFORF1919 | 4 | TFORF1887 | 3 | TFORF2971 | 3 |
| TFORF0285 | 3 | TFORF1920 | 4 | TFORF1888 | 3 | TFORF2972 | 3 |
| TFORF0286 | 3 | TFORF3473 | 4 | TFORF3282 | 3 | TFORF3015 | 3 |
| TFORF0287 | 3 | TFORF1999 | 4 | TFORF0459 | 3 | TFORF0499 | 3 |
| TFORF0288 | 3 | TFORF0960 | 3 | TFORF0460 | 3 | TFORFO500 | 3 |
| TFORF0289 | 3 | TFORF0961 | 3 | TFORF0461 | 3 | TFORFO731 | 3 |
| TFORF0290 | 3 | TFORF0962 | 3 | TFORF0462 | 3 | TFORF2274 | 3 |
| TFORF0291 | 3 | TFORF0963 | 3 | TFORF2305 | 3 | TFORF2275 | 3 |
| TFORF0235 | 3 | TFORF0964 | 3 | TFORF2306 | 3 | TFORF2276 | 3 |
| TFORF0236 | 3 | TFORF3192 | 3 | TFORF2307 | 3 | TFORF2881 | 3 |
| TFORF1151 | 3 | TFORF2240 | 3 | TFORF2308 | 3 | TFORF2882 | 3 |
| TFORF1152 | 3 | TFORF2241 | 3 | TFORF2309 | 3 | TFORF2553 | 2 |
| TFORF0923 | 3 | TFORF1525 | 3 | TFORF2310 | 3 | TFORF2554 | 2 |
| TFORF0924 | 3 | TFORF0099 | 3 | TFORF3461 | 3 | TFORF3036 | 2 |
| TFORF0925 | 3 | TFORF3398 | 3 | TFORF0079 | 3 | TFORF2545 | 2 |
| TFORF0499 | 3 | TFORF2841 | 3 | TFORF0080 | 3 | TFORF3412 | 2 |
| TFORF0500 | 3 | TFORF2842 | 3 | TFORF0323 | 3 | TFORF1125 | 2 |
| TFORF3498 | 2 | TFORF2070 | 3 | TFORF0324 | 3 | TFORF0959 | 2 |
| TFORF2593 | 2 | TFORF2071 | 3 | TFORF0325 | 3 | TFORF3527 | 2 |
| TFORF1207 | 2 | TFORF2072 | 3 | TFORF1033 | 3 | TFORF3075 | 2 |
| TFORF1208 | 2 | TFORF2073 | 3 | TFORF2401 | 3 | TFORF2609 | 2 |
| TFORF0954 | 2 | TFORF2074 | 3 | TFORF2747 | 3 | TFORF2610 | 2 |
| TFORF0955 | 2 | TFORF2075 | 3 | TFORF3482 | 3 | TFORF3366 | 2 |
| TFORF3533 | 2 | TFORF2076 | 3 | TFORF0006 | 3 | TFORF3274 | 2 |
| TFORF2700 | 2 | TFORF2077 | 3 | TFORF0007 | 3 | TFORF3275 | 2 |
| TFORF2701 | 2 | TFORF0235 | 3 | TFORF1889 | 3 | TFORF3224 | 2 |
| TFORF2702 | 2 | TFORF0236 | 3 | TFORF1890 | 3 | TFORF1684 | 2 |
| TFORF2942 | 2 | TFORF2085 | 3 | TFORF1891 | 3 | TFORF1685 | 2 |
| TFORF2943 | 2 | TFORF2086 | 3 | TFORF1892 | 3 | TFORF1686 | 2 |
| TFORF2234 | 2 | TFORF2087 | 3 | TFORF1922 | 3 | TFORF1687 | 2 |
| TFORF2161 | 2 | TFORF2088 | 3 | TFORF1923 | 3 | TFORF2991 | 2 |
| TFORF2162 | 2 | TFORF2059 | 3 | TFORF0089 | 3 | TFORF0989 | 2 |
| TFORF2849 | 2 | TFORF0675 | 3 | TFORF0090 | 3 | TFORF2384 | 2 |
| TFORF2850 | 2 | TFORF0676 | 3 | TFORF0091 | 3 | TFORF2385 | 2 |
| TFORF2851 | 2 | TFORF0677 | 3 | TFORF0092 | 3 | TFORF2908 | 2 |
| TFORF2852 | 2 | TFORF2633 | 3 | TFORF0093 | 3 | TFORF2909 | 2 |
| TFORF2853 | 2 | TFORF2634 | 3 | TFORF2096 | 3 | TFORF2910 | 2 |
| TFORF3459 | 2 | TFORF2635 | 3 | TFORF2097 | 3 | TFORF2911 | 2 |
| TFORF1658 | 2 | TFORF2636 | 3 | TFORF2098 | 3 | TFORF2912 | 2 |
| TFORF1659 | 2 | TFORF2637 | 3 | TFORF3212 | 3 | TFORF2978 | 2 |
| TFORF1660 | 2 | TFORF3178 | 3 | TFORF1873 | 2 | TFORF2979 | 2 |
| TFORF3247 | 2 | TFORF2152 | 2 | TFORF1874 | 2 | TFORF0628 | 2 |
| TFORF0814 | 2 | TFORF2153 | 2 | TFORF3273 | 2 | TFORF0629 | 2 |
| TFORF1136 | 2 | TFORF2154 | 2 | TFORF0467 | 2 | TFORF0630 | 2 |
| TFORF1137 | 2 | TFORF2155 | 2 | TFORF0660 | 2 | TFORF2007 | 2 |
| TFORF1138 | 2 | TFORF2586 | 2 | TFORF0661 | 2 | TFORF3453 | 2 |
| TFORF1139 | 2 | TFORF0656 | 2 | TFORF0789 | 2 | TFORF2862 | 2 |
| TFORF1938 | 2 | TFORF0657 | 2 | TFORF2873 | 2 | TFORF2863 | 2 |
| TFORF1939 | 2 | TFORF0658 | 2 | TFORF2874 | 2 | TFORF2658 | 2 |
| TFORF2340 | 2 | TFORF0064 | 2 | TFORF1096 | 2 | TFORF2659 | 2 |
| TFORF2341 | 2 | TFORF0065 | 2 | TFORF3156 | 2 | TFORF2656 | 2 |
| TFORF2342 | 2 | TFORF2873 | 2 | TFORF1615 | 2 | TFORF2657 | 2 |
| TFORF2343 | 2 | TFORF2874 | 2 | TFORF2991 | 2 | TFORF3358 | 2 |
| TFORF2344 | 2 | TFORF1846 | 2 | TFORF0075 | 2 | TFORF0244 | 2 |
| TFORF2345 | 2 | TFORF1847 | 2 | TFORF0076 | 2 | TFORF0417 | 2 |
| TFORF2346 | 2 | TFORF1848 | 2 | TFORF0077 | 2 | TFORF0508 | 2 |
| TFORF3303 | 2 | TFORF1849 | 2 | TFORF0078 | 2 | TFORF0509 | 2 |
| TFORF3304 | 2 | TFORF2078 | 2 | TFORF3405 | 2 | TFORF1922 | 2 |
| TFORF0769 | 2 | TFORF2079 | 2 | TFORF3505 | 2 | TFORF1923 | 2 |
| TFORF0770 | 2 | TFORF2080 | 2 | TFORF2855 | 2 | TFORF3084 | 2 |
| TFORF0771 | 2 | TFORF2081 | 2 | TFORF2856 | 2 | TFORF1994 | 2 |
| TFORF2913 | 2 | TFORF2782 | 2 | TFORF2340 | 2 | TFORF2096 | 2 |
| TFORF2914 | 2 | TFORF1507 | 2 | TFORF2341 | 2 | TFORF2097 | 2 |
| TFORF2915 | 2 | TFORF3136 | 2 | TFORF2342 | 2 | TFORF2098 | 2 |
| TFORF2916 | 2 | TFORF3137 | 2 | TFORF2343 | 2 | TFORF3212 | 2 |
| TFORF2917 | 2 | TFORF0001 | 2 | TFORF2344 | 2 | | |
| TFORF2918 | 2 | TFORF0002 | 2 | TFORF2345 | 2 | | |
| TFORF2919 | 2 | TFORF0003 | 2 | TFORF2346 | 2 | | |
| TFORF2920 | 2 | TFORF0004 | 2 | TFORF3303 | 2 | | |
| TFORF3427 | 2 | TFORF0005 | 2 | TFORF3304 | 2 | | |
| TFORF0366 | 2 | TFORF2849 | 2 | TFORF2921 | 2 | | |
| TFORF0367 | 2 | TFORF2850 | 2 | TFORF2922 | 2 | | |
| TFORF0368 | 2 | TFORF2851 | 2 | TFORF2923 | 2 | | |
| TFORF0369 | 2 | TFORF2852 | 2 | TFORF2924 | 2 | | |
| TFORF0370 | 2 | TFORF2853 | 2 | TFORF2913 | 2 | | |
| TFORF0371 | 2 | TFORF0576 | 2 | TFORF2914 | 2 | | |
| TFORF3002 | 2 | TFORF3283 | 2 | TFORF2915 | 2 | | |
| TFORF2016 | 2 | TFORF3288 | 2 | TFORF2916 | 2 | | |
| TFORF2017 | 2 | TFORF0879 | 2 | TFORF2917 | 2 | | |
| TFORF3348 | 2 | TFORF0880 | 2 | TFORF2918 | 2 | | |
| TFORF1054 | 2 | TFORF0881 | 2 | TFORF2919 | 2 | | |
| TFORF1055 | 2 | TFORF0884 | 2 | TFORF2920 | 2 | | |
| TFORF1708 | 2 | TFORF3239 | 2 | TFORF3427 | 2 | | |
| TFORF1674 | 2 | TFORF3037 | 2 | TFORF2023 | 2 | | |
| TFORF1675 | 2 | TFORF3038 | 2 | TFORF2024 | 2 | | |
| TFORF1676 | 2 | TFORF0756 | 2 | TFORF2025 | 2 | | |
| TFORF1677 | 2 | TFORF2772 | 2 | TFORF2026 | 2 | | |
| TFORF1678 | 2 | TFORF2773 | 2 | TFORF0731 | 2 | | |
| TFORF1679 | 2 | TFORF0988 | 2 | TFORF1217 | 2 | | |
| TFORF2311 | 2 | TFORF0989 | 2 | TFORF1218 | 2 | | |
| TFORF3022 | 2 | TFORF1769 | 2 | TFORF0610 | 2 | | |
| TFORF0109 | 2 | TFORF1770 | 2 | TFORF0611 | 2 | | |
| TFORF0104 | 2 | TFORF2402 | 2 | TFORF0612 | 2 | | |
| TFORF3314 | 2 | TFORF2403 | 2 | TFORF3253 | 2 | | |
| TFORF1047 | 2 | TFORF3418 | 2 | TFORF2597 | 2 | | |
| TFORF0584 | 2 | TFORF2927 | 2 | TFORF2598 | 2 | | |
| TFORF3123 | 2 | TFORF2928 | 2 | TFORF2599 | 2 | | |
| TFORF1973 | 2 | TFORF2929 | 2 | TFORF2987 | 2 | | |
| TFORF1974 | 2 | TFORF2930 | 2 | TFORF2258 | 2 | | |
| | | TFORF2016 | 2 | TFORF1696 | 2 | | |
| | | TFORF2017 | 2 | TFORF2294 | 2 | | |
| | | TFORF3348 | 2 | TFORF2245 | 2 | | |
| | | TFORF1316 | 2 | TFORF2246 | 2 | | |
| | | TFORF1317 | 2 | TFORF2247 | 2 | | |
| | | TFORF1674 | 2 | TFORF2248 | 2 | | |
| | | TFORF1675 | 2 | TFORF2249 | 2 | | |
| | | TFORF1676 | 2 | TFORF1902 | 2 | | |
| | | TFORF1677 | 2 | TFORF1903 | 2 | | |
| | | TFORF1678 | 2 | TFORF1904 | 2 | | |
| | | TFORF1679 | 2 | TFORF1905 | 2 | | |
| | | TFORF0379 | 2 | TFORF1906 | 2 | | |
| | | TFORF0380 | 2 | TFORF1907 | 2 | | |
| | | TFORF0381 | 2 | TFORF1908 | 2 | | |
| | | TFORF0382 | 2 | TFORF1909 | 2 | | |
| | | TFORF0383 | 2 | TFORF1910 | 2 | | |
| | | TFORF0384 | 2 | TFORF1911 | 2 | | |
| | | TFORF1652 | 2 | TFORF1912 | 2 | | |
| | | TFORF1653 | 2 | TFORF1913 | 2 | | |
| | | TFORF1902 | 2 | TFORF3417 | 2 | | |
| | | TFORF1903 | 2 | TFORF1457 | 2 | | |
| | | TFORF1904 | 2 | TFORF1458 | 2 | | |
| | | TFORF1905 | 2 | TFORF2934 | 2 | | |
| | | TFORF1906 | 2 | TFORF3376 | 2 | | |
| | | TFORF1907 | 2 | TFORF3084 | 2 | | |
| | | TFORF1908 | 2 | TFORF2398 | 2 | | |
| | | TFORF1909 | 2 | TFORF2388 | 2 | | |
| | | TFORF1910 | 2 | TFORF1988 | 2 | | |
| | | TFORF1911 | 2 | TFORF0009 | 2 | | |
| | | TFORF1912 | 2 | | | | |
| | | TFORF1913 | 2 | | | | |
| | | TFORF3417 | 2 | | | | |
| | | TFORF2689 | 2 | | | | |
| | | TFORF2690 | 2 | | | | |
| | | TFORF2691 | 2 | | | | |
| | | TFORF2985 | 2 | | | | |
| | | TFORF1039 | 2 | | | | |
| | | TFORF1040 | 2 | | | | |
| | | TFORF1041 | 2 | | | | |
| | | TFORF1042 | 2 | | | | |
| | | TFORF1419 | 2 | | | | |
| | | TFORF3529 | 2 | | | | |
| | | TFORF2096 | 2 | | | | |
| | | TFORF2097 | 2 | | | | |
| | | TFORF2098 | 2 | | | | |
| | | TFORF3212 | 2 | | | | |

TABLE 8
Genes with TF ChIP-seq peaks (see, U.S. provisional application 63/219,705
filed Jul. 8, 2021). For each TF, genes with transcriptional start sites that
that were within 10 kb of the TF ChIP-seq peak region identified by MACS.

| | | |
|---|---|---|
| Endothelial TFs | Epithelial TFs | Limb muscle TFs |

| Table 3 Name | nDatasets | Table 3 Name | nDatasets | Table 3 Name | nDatasets |
|---|---|---|---|---|---|
| TFORF1852 | 5 | TFORF2209 | 2 | TFORF2946 | 4 |
| TFORF2926 | 4 | TFORF2210 | 2 | TFORF0526 | 4 |
| TFORF1995 | 4 | TFORF2211 | 2 | TFORF0527 | 4 |
| TFORF3081 | 3 | TFORF2212 | 2 | TFORF3020 | 4 |
| TFORF0960 | 3 | TFORF2694 | 2 | TFORF0956 | 3 |
| TFORF0961 | 3 | TFORF2695 | 2 | TFORF0101 | 2 |
| TFORF0962 | 3 | TFORF2696 | 2 | TFORF3361 | 2 |
| TFORF0963 | 3 | TFORF3109 | 2 | TFORF2641 | 2 |
| TFORF0964 | 3 | TFORF3366 | 2 | TFORF2642 | 2 |
| TFORF3192 | 3 | TFORF2163 | 2 | TFORF2643 | 2 |
| TFORF2260 | 3 | TFORF2164 | 2 | TFORF2644 | 2 |
| TFORF0171 | 3 | TFORF2165 | 2 | TFORF2645 | 2 |
| TFORF3075 | 2 | TFORF2166 | 2 | TFORF2646 | 2 |
| TFORF2801 | 2 | TFORF2167 | 2 | TFORF2647 | 2 |
| TFORF2802 | 2 | TFORF2168 | 2 | TFORF2648 | 2 |
| TFORF2803 | 2 | TFORF0689 | 2 | TFORF2016 | 2 |
| TFORF2804 | 2 | TFORF0690 | 2 | TFORF2017 | 2 |
| TFORF0024 | 2 | TFORF2954 | 2 | TFORF3348 | 2 |
| TFORF1140 | 2 | TFORF2464 | 2 | TFORF1219 | 2 |
| TFORF1141 | 2 | TFORF2465 | 2 | TFORF1919 | 2 |
| TFORF1142 | 2 | TFORF0056 | 2 | TFORF1920 | 2 |
| TFORF1143 | 2 | TFORF0057 | 2 | TFORF3473 | 2 |
| TFORF0987 | 2 | TFORF0058 | 2 | TFORF2568 | 2 |
| TFORF0978 | 2 | | | TFORF2569 | 2 |
| TFORF0979 | 2 | | | TFORF2570 | 2 |
| TFORF0980 | 2 | | | TFORF2996 | 2 |
| TFORF3464 | 2 | | | | |
| TFORF0587 | 2 | | | | |
| TFORF3163 | 2 | | | | |
| TFORF2537 | 2 | | | | |

| Endothelial Markers | | Epithelial Markers | | Limb muscle Markers | |
|---|---|---|---|---|---|
| Gene | nDatasets | Gene | nDatasets | Gene | nDatasets |
| CLDN5 | 6 | KRT8 | 5 | FLNC | 3 |
| CD34 | 6 | KRT18 | 4 | VCAM1 | 3 |
| TEK | 6 | CLDN6 | 4 | TNFRSF12A | 3 |
| CDH5 | 5 | KRT7 | 4 | CD82 | 3 |
| RAMP2 | 5 | EPCAM | 4 | SDC4 | 3 |
| FLT1 | 5 | KRT19 | 3 | ERRFI1 | 2 |
| PECAM1 | 5 | CDH1 | 3 | MEG3 | 2 |
| ECSCR | 5 | CLDN7 | 3 | CDKN1A | 2 |
| ICAM2 | 5 | SPINT2 | 3 | ITGA7 | 2 |
| EGFL7 | 5 | SPINT1 | 3 | PDLIM4 | 2 |
| ESAM | 5 | KRT20 | 3 | GPX3 | 2 |
| ARHGEF15 | 4 | AP1M2 | 2 | FBN2 | 2 |
| EMCN | 4 | PPAP2A | 2 | SYTL2 | 2 |
| SHE | 4 | ADH6A | 2 | ASB5 | 2 |
| CD93 | 4 | CBLC | 2 | VGLL2 | 2 |
| FLT4 | 4 | CNDP2 | 2 | VGLL3 | 2 |
| MMRN2 | 4 | MYL12B | 2 | ODC1 | 2 |
| GIMAP4 | 4 | CLEC2H | 2 | SEMA6A | 2 |
| GNG11 | 4 | ITGA3 | 2 | MUSK | 2 |
| PLVAP | 4 | APOA4 | 2 | CRLF1 | 2 |
| KLHL4 | 4 | LSR | 2 | FGFR4 | 2 |
| CTLA2A | 4 | REG3A | 2 | FGFR1 | 2 |
| EXOC3L | 4 | MOGAT2 | 2 | CHODL | 2 |
| MYCT1 | 4 | SLC39A4 | 2 | PDPN | 2 |
| GPR116 | 3 | CYP2D26 | 2 | NPPC | 2 |
| RASGRP3 | 3 | KRT14 | 2 | CHRNB1 | 2 |
| MYZAP | 3 | MYH14 | 2 | CD63 | 2 |
| SRGAP1 | 3 | COX7A1 | 2 | CDH15 | 2 |
| TFPI | 3 | ENPEP | 2 | GAL | 2 |
| SCARF1 | 3 | MME | 2 | HNRNPA2B1 | 2 |
| SH2D3C | 3 | CYP3A13 | 2 | FAM132B | 2 |
| ACER2 | 3 | IGSF9 | 2 | RPL6 | 2 |
| SPTBN1 | 3 | RDH7 | 2 | DES | 2 |
| N4BP3 | 3 | CYP2C65 | 2 | NACA | 2 |
| NOS3 | 3 | CYP2C66 | 2 | TENM4 | 2 |
| CD40 | 3 | HSD17B6 | 2 | ARL4D | 2 |
| CMTM3 | 3 | CYB5B | 2 | | |
| PRCP | 3 | GSTM6 | 2 | | |
| PLXND1 | 3 | GSTA1 | 2 | | |
| SH3BP5 | 3 | DHRS1 | 2 | | |
| VIM | 3 | LAD1 | 2 | | |
| CTLA2B | 3 | SLC2A2 | 2 | | |
| CALCRL | 3 | XPNPEP1 | 2 | | |
| ENG | 3 | OCM | 2 | | |
| PCDH12 | 3 | RETSAT | 2 | | |
| S1PR1 | 3 | EPHX2 | 2 | | |
| FGD5 | 3 | CAR4 | 2 | | |
| ARAP3 | 3 | FABP1 | 2 | | |
| CD38 | 3 | SLC5A1 | 2 | | |
| CAV1 | 3 | FRK | 2 | | |
| THSD1 | 3 | CKMT1 | 2 | | |
| TIE1 | 3 | CYP3A25 | 2 | | |
| RASIP1 | 3 | SLC16A1 | 2 | | |
| TMEM204 | 3 | PHGR1 | 2 | | |
| BC028528 | 3 | CKB | 2 | | |
| ANXA3 | 3 | APOC3 | 2 | | |
| FAM43A | 3 | 2200002D01RIK | 2 | | |
| KANK3 | 3 | NAALADL1 | 2 | | |
| KCNE3 | 3 | UPK1B | 2 | | |
| PLK2 | 3 | GSTA4 | 2 | | |
| EDN1 | 3 | GLT28D2 | 2 | | |
| KLHL6 | 3 | MYO5C | 2 | | |
| ACVRL1 | 3 | UGDH | 2 | | |
| RHOJ | 3 | PRLR | 2 | | |
| GRAP | 3 | XDH | 2 | | |
| VAMP5 | 3 | VIL1 | 2 | | |
| PTPRB | 3 | MAL2 | 2 | | |
| GPM6B | 2 | H2-Q2 | 2 | | |
| GPR182 | 2 | LCT | 2 | | |
| APBB2 | 2 | MS4A10 | 2 | | |
| DYSF | 2 | KRT5 | 2 | | |
| TMEM184B | 2 | CEACAM20 | 2 | | |
| GAP43 | 2 | SLC34A2 | 2 | | |
| CSGALNACT1 | 2 | GSTM3 | 2 | | |
| FMNL3 | 2 | WNT6 | 2 | | |
| FLNB | 2 | WNT4 | 2 | | |
| TMEM88 | 2 | RIPK4 | 2 | | |
| MCAM | 2 | GPD1 | 2 | | |
| GJA4 | 2 | LPGAT1 | 2 | | |
| IL2RG | 2 | ALDH1A1 | 2 | | |
| PRKD2 | 2 | ANPEP | 2 | | |
| NPR1 | 2 | ALDOB | 2 | | |
| PFN1 | 2 | CASP6 | 2 | | |
| TSPAN18 | 2 | FAM213A | 2 | | |
| TMEM173 | 2 | FAM160A1 | 2 | | |
| CLEC1A | 2 | KHK | 2 | | |
| ELTD1 | 2 | RBP2 | 2 | | |
| ADORA2A | 2 | PRAP1 | 2 | | |
| NID1 | 2 | TREH | 2 | | |
| ITPKB | 2 | ACSL5 | 2 | | |
| TDRP | 2 | GRB7 | 2 | | |
| IGF1 | 2 | DPEP1 | 2 | | |
| APLNR | 2 | ABCB1A | 2 | | |
| RALB | 2 | GRAMD3 | 2 | | |
| HDAC7 | 2 | SLC26A3 | 2 | | |
| FABP4 | 2 | WFDC2 | 2 | | |
| HSPA12B | 2 | BCAM | 2 | | |
| HBB-BS | 2 | MEP1A | 2 | | |
| SIPA1 | 2 | SLC51A | 2 | | |
| VGLL4 | 2 | SLC51B | 2 | | |
| DLL4 | 2 | MTTP | 2 | | |
| OIT3 | 2 | SULT1B1 | 2 | | |
| TJP1 | 2 | ACE | 2 | | |
| LPAR6 | 2 | EPHA1 | 2 | | |
| AMOTL1 | 2 | CYB5R3 | 2 | | |
| STAB1 | 2 | SLC7A9 | 2 | | |
| ECE1 | 2 | SLC13A1 | 2 | | |
| ICA1 | 2 | FERMT1 | 2 | | |
| PRKCH | 2 | GDA | 2 | | |
| CARD10 | 2 | SCP2 | 2 | | |
| ROCK2 | 2 | BEX1 | 2 | | |
| LYVE1 | 2 | TMPRSS2 | 2 | | |
| RAPGEF4 | 2 | AMN | 2 | | |
| CAV2 | 2 | | | | |
| PPP1R2 | 2 | | | | |
| MAP3K3 | 2 | | | | |
| STAP2 | 2 | | | | |
| CTSW | 2 | | | | |
| ADCY4 | 2 | | | | |
| PTPN12 | 2 | | | | |
| PPFIBP1 | 2 | | | | |
| CCM2L | 2 | | | | |
| FKBP1A | 2 | | | | |
| HSPG2 | 2 | | | | |
| KIF26A | 2 | | | | |
| SLC31A2 | 2 | | | | |
| LIMCH1 | 2 | | | | |
| MALL | 2 | | | | |
| GIMAP8 | 2 | | | | |
| GIMAP6 | 2 | | | | |
| HAPLN1 | 2 | | | | |
| GRK5 | 2 | | | | |
| VEGFC | 2 | | | | |
| MYO1C | 2 | | | | |
| CD81 | 2 | | | | |
| CCDC85B | 2 | | | | |
| COLGALT2 | 2 | | | | |
| MAP4K2 | 2 | | | | |
| YES1 | 2 | | | | |
| RGS3 | 2 | | | | |
| PEAK1 | 2 | | | | |
| COL18A1 | 2 | | | | |
| PIEZO2 | 2 | | | | |
| PKN3 | 2 | | | | |
| CYYR1 | 2 | | | | |
| CASP7 | 2 | | | | |
| CNRIP1 | 2 | | | | |
| FXYD5 | 2 | | | | |
| S100A16 | 2 | | | | |
| MRC1 | 2 | | | | |
| DENND3 | 2 | | | | |
| ARHGAP27 | 2 | | | | |
| VWA1 | 2 | | | | |
| AU021092 | 2 | | | | |
| KCTD12 | 2 | | | | |
| ROBO4 | 2 | | | | |
| AFAP1L1 | 2 | | | | |
| USHBP1 | 2 | | | | |
| ABCB1A | 2 | | | | |
| PREX2 | 2 | | | | |
| PGM1 | 2 | | | | |
| ITPR3 | 2 | | | | |
| GNAI2 | 2 | | | | |
| RASSF2 | 2 | | | | |
| LRRC8C | 2 | | | | |
| C130074G19RIK | 2 | | | | |
| EOGT | 2 | | | | |
| PODXL | 2 | | | | |
| MSN | 2 | | | | |
| SLC9A3R2 | 2 | | | | |
| DOK4 | 2 | | | | |
| GNGT2 | 2 | | | | |
| ADGRF5 | 2 | | | | |
| PCDH1 | 2 | | | | |
| LY6C1 | 2 | | | | |
| ESM1 | 2 | | | | |

TABLE 9
Lists of marker genes and TFs for applying TF screening to additional cell types. For some additional cell
types, Applicants have recommended lists of marker genes and TFs based on published RNA-seq datasets.

| Cardiomyocte Markers | | Hepatocyte Markers | | HSC Markers | | Oligodendrocyte Markers | |
|---|---|---|---|---|---|---|---|
| Gene | nDatasets | Gene | nDatasets | Gene | nDatasets | Gene | nDatasets |
| MYL4 | 8 | ALB | 9 | MPL | 5 | MOG | 10 |
| MYH7 | 8 | ASS1 | 6 | GUCY1A3 | 4 | MAG | 8 |
| NEBL | 7 | APOA1 | 6 | PROCR | 4 | MAL | 8 |
| NPPA | 7 | AGT | 5 | KIT | 4 | PDGFRA | 8 |
| TNNC1 | 7 | GC | 5 | TSPAN32 | 4 | OPALIN | 8 |
| MYH6 | 7 | MGST1 | 5 | SERPINF1 | 4 | CLDN11 | 8 |
| CSRP3 | 7 | TTR | 5 | SPNS2 | 3 | MOBP | 7 |
| MYL7 | 7 | PCK1 | 5 | RAB27B | 3 | PLEKHH1 | 7 |
| PLN | 7 | KNG1 | 5 | PPP1R11 | 3 | ITPR2 | 7 |
| TPM1 | 6 | CYP2E1 | 5 | DEF6 | 3 | QDPR | 7 |
| SMPX | 6 | HAL | 5 | GCNT2 | 3 | CNP | 7 |
| NPPB | 6 | FN1 | 5 | RBP1 | 3 | ERMN | 7 |
| ACTC1 | 6 | APOH | 5 | DAPP1 | 3 | TNR | 7 |
| MYL2 | 6 | AHSG | 5 | BEX1 | 3 | ASPA | 7 |
| CRYAB | 6 | ASL | 5 | IGF1R | 3 | PLLP | 6 |
| TTN | 5 | ALDOB | 5 | SLC18A2 | 3 | SIRT2 | 6 |
| SH3BGR | 5 | APOA2 | 4 | ZFP467 | 3 | LHFPL3 | 6 |
| PPP1R3C | 5 | FTCD | 4 | OSBPL1A | 3 | PTPRZ1 | 6 |
| ACTN2 | 5 | SC5D | 4 | ANGPT1 | 3 | CDK18 | 6 |
| FITM1 | 5 | CTH | 4 | ENG | 3 | FA2H | 6 |
| RRAD | 5 | GPT2 | 4 | F10 | 3 | PCDH15 | 6 |
| MYBPC3 | 5 | SULT1A1 | 4 | CAR2 | 3 | HAPLN2 | 6 |
| FHL2 | 5 | TCEA3 | 4 | PRKCA | 3 | SLC44A1 | 6 |
| MDH1 | 5 | PIPOX | 4 | PDZK1IP1 | 3 | BCAN | 6 |
| ADPRHL1 | 5 | SPP2 | 4 | WAS | 3 | ANLN | 6 |
| CSRP2 | 5 | DPYS | 4 | MSI2 | 3 | UGT8A | 6 |
| TNNT2 | 5 | CFI | 4 | GSE1 | 3 | APOD | 6 |
| LRRC10 | 5 | CFB | 4 | RBPMS | 3 | CNTN2 | 6 |
| TNNI3 | 5 | HAMP | 4 | DUSP2 | 3 | GPR17 | 6 |
| TNNI1 | 5 | ITIH3 | 4 | LYZ2 | 3 | LG13 | 6 |
| MYL3 | 5 | AFM | 4 | CDKN1C | 3 | EPN2 | 6 |
| MYL9 | 5 | PROZ | 4 | APOE | 3 | TMEFF2 | 6 |
| HSPB7 | 5 | AZGP1 | 4 | VWF | 3 | HHIP | 6 |
| HSPB2 | 5 | PROC | 4 | LTB | 3 | PLP1 | 6 |
| ANKRD1 | 5 | EPHX2 | 4 | LGALS3BP | 3 | ABCA2 | 6 |
| MYLK3 | 4 | ETFA | 4 | GNG11 | 3 | TMEM88B | 6 |
| SLC25A4 | 4 | ASGR2 | 4 | IFITM1 | 3 | MBP | 6 |
| TCAP | 4 | GCGR | 4 | CTLA2A | 3 | OPHN1 | 5 |
| VSNL1 | 4 | PAH | 4 | CDC42BPA | 3 | PHLDB1 | 5 |
| PPP1R14C | 4 | MASP2 | 4 | BLVRB | 3 | SECISBP2L | 5 |
| ENO3 | 4 | ACOX2 | 4 | VAMP8 | 3 | FYN | 5 |
| SORBS2 | 4 | DMGDH | 4 | CBFA2T3 | 3 | KANK1 | 5 |
| LDB3 | 4 | C3 | 4 | TNIP3 | 3 | GJC3 | 5 |
| NEXN | 4 | HMGCS2 | 4 | RBM38 | 3 | TUBB4A | 5 |
| APOBEC2 | 4 | SORD | 4 | GIMAP5 | 3 | GJB1 | 5 |
| MYOM1 | 4 | CP | 4 | MLLT3 | 3 | PPP1R14A | 5 |
| DES | 4 | HPX | 4 | MYCT1 | 3 | CASK | 5 |
| COX6A2 | 4 | AADAC | 4 | MAGED2 | 3 | EDIL3 | 5 |
| PDLIM5 | 4 | HPN | 4 | TRIM47 | 3 | VCAN | 5 |
| TRIM55 | 4 | EBP | 4 | ESAM | 3 | CHN2 | 5 |
| COX5A | 4 | FBP1 | 4 | KRT18 | 3 | DOCK5 | 5 |
| POPDC2 | 4 | RBP4 | 4 | SGMS1 | 3 | FRMD4B | 5 |
| SMYD1 | 4 | ACSL1 | 4 | ITIH5 | 3 | SERPINE2 | 5 |
| PGAM2 | 4 | HP | 4 | GABARAPL1 | 3 | SLC35F1 | 5 |
| TRIM63 | 4 | GNMT | 4 | PIK3IP1 | 3 | NTM | 5 |
| THBS4 | 4 | ERRFI1 | 4 | NRGN | 3 | SNX22 | 5 |
| AGL | 3 | HSD17B13 | 4 | AFAP1L1 | 3 | NEU4 | 5 |
| ASB2 | 3 | APOC4 | 4 | RAB38 | 3 | AATK | 5 |
| KCNG2 | 3 | ECHDC2 | 4 | LAPTM5 | 2 | DOCK10 | 5 |
| BVES | 3 | AC01 | 4 | SLC50A1 | 2 | BCAS1 | 5 |
| LRRC39 | 3 | SLC27A2 | 4 | CAMK1 | 2 | CRYAB | 5 |
| ABRA | 3 | ABAT | 4 | GRINA | 2 | ENPP6 | 5 |
| CKM | 3 | BHMT2 | 4 | FAM84B | 2 | ENPP2 | 5 |
| SLC25A5 | 3 | GJB1 | 4 | SNN | 2 | TSPAN2 | 5 |
| TMEM176B | 3 | GRHPR | 4 | TRAPPC5 | 2 | LUZP2 | 5 |
| CHCHD10 | 3 | F12 | 4 | MGST1 | 2 | ZFP488 | 4 |
| VDAC2 | 3 | SERPINA10 | 4 | GPRASP2 | 2 | PPP2R2B | 4 |
| COL1A1 | 3 | HAAO | 4 | DSEL | 2 | DBNDD2 | 4 |
| TAGLN | 3 | AMDHD1 | 4 | THSD1 | 2 | KNDC1 | 4 |
| MPPED2 | 3 | F13B | 4 | COL4A2 | 2 | KLK6 | 4 |
| RASSF5 | 3 | ACOX1 | 4 | COL4A1 | 2 | LSAMP | 4 |
| ADM | 3 | BDH1 | 4 | CHST2 | 2 | NXPH1 | 4 |
| CCDC141 | 3 | GCKR | 4 | RPS6KA6 | 2 | SEZ6L | 4 |
| ALPK2 | 3 | GOT2 | 4 | KHK | 2 | ALCAM | 4 |
| MB | 3 | SLC25A47 | 4 | NPHP1 | 2 | TRIO | 4 |
| CNN1 | 3 | SDC4 | 4 | IGF2R | 2 | SMOC1 | 4 |
| HRC | 3 | SERPINA6 | 4 | ART4 | 2 | NINJ2 | 4 |
| GYG | 3 | ALDH6A1 | 4 | SULT1A1 | 2 | POLR3E | 4 |
| TECRL | 3 | AKR1D1 | 4 | CCDC112 | 2 | NTRK3 | 4 |
| IDH2 | 3 | ANGPTL3 | 4 | CXXC5 | 2 | ARHGAP23 | 4 |
| RBPMS | 3 | TST | 4 | IPO11 | 2 | PPP1R16B | 4 |
| PPP1R12B | 3 | APOF | 4 | SASH3 | 2 | ABHD2 | 4 |
| BMP10 | 3 | APOB | 4 | TRIM16 | 2 | OPCML | 4 |
| IDH3A | 3 | APOM | 4 | PPP2R3A | 2 | GJC2 | 4 |
| JPH2 | 3 | MST1 | 4 | CSAD | 2 | TRAF4 | 4 |
| ACTA2 | 3 | ALDH8A1 | 4 | SERINC3 | 2 | NOVA1 | 4 |
| MYO18B | 3 | LIPC | 4 | PINK1 | 2 | PTGDS | 4 |
| XIRP1 | 3 | HGFAC | 4 | PRKG1 | 2 | SORT1 | 4 |
| FBXO32 | 3 | VTN | 4 | PPP1R9A | 2 | SLC1A2 | 4 |
| ANXA2 | 3 | PON1 | 4 | IGF2 | 2 | OSBPL1A | 4 |
| COX7A1 | 3 | PXMP2 | 4 | PHLDB1 | 2 | DNAJB2 | 4 |
| CAV1 | 3 | OTC | 4 | GJA1 | 2 | SLC24A2 | 4 |
| PRELID2 | 3 | SERPINC1 | 4 | MFNG | 2 | GAL3ST1 | 4 |
| SYNPO2L | 3 | SHMT1 | 4 | TXNIP | 2 | MATN4 | 4 |
| S100A10 | 3 | BHMT | 4 | ITGA2B | 2 | MARCKS | 4 |
| FSD2 | 3 | FAH | 4 | KTN1 | 2 | ERBB3 | 4 |
| MYOZ2 | 3 | UPB1 | 4 | PCBD1 | 2 | TMEM108 | 4 |
| CCND2 | 3 | HADH | 4 | MMRN1 | 2 | NKAIN2 | 4 |
| PDLIM3 | 3 | PANK1 | 4 | ARHGAP31 | 2 | EFHD1 | 4 |
| CRIP2 | 3 | F2 | 4 | CD34 | 2 | ZDHHC20 | 4 |
| POPDC3 | 3 | F7 | 4 | IGF2BP2 | 2 | NRCAM | 4 |
| FABP3 | 3 | GSTZ1 | 4 | MBNL1 | 2 | BRINP3 | 4 |
| TRDN | 3 | ACAA2 | 4 | PER3 | 2 | SLC5A11 | 4 |
| SLC8A1 | 3 | HGD | 4 | TMEM176A | 2 | TRF | 4 |
| CYCS | 3 | MAT1A | 4 | TMEM176B | 2 | CARNS1 | 4 |
| TMOD1 | 3 | SLC25A13 | 4 | PTPN18 | 2 | PACS2 | 4 |
| RYR2 | 3 | FGG | 4 | F2R | 2 | HIPK2 | 4 |
| DSTN | 3 | FGA | 4 | H19 | 2 | SEMA4D | 4 |
| UNC45B | 3 | FGB | 4 | LARGE | 2 | MMP16 | 4 |
| LDHA | 3 | SERPIND1 | 4 | ECSCR | 2 | TMEM125 | 4 |
| LINC00881 | 3 | MAOB | 4 | CX3CL1 | 2 | CPOX | 4 |
| ATP2A2 | 2 | FABP1 | 4 | ZFP532 | 2 | NLGN1 | 4 |
| ITGA6 | 2 | ITIH1 | 4 | OBSL1 | 2 | PLEKHA1 | 4 |
| H19 | 2 | ITIH2 | 4 | SRC | 2 | CNTN1 | 4 |
| COL4A5 | 2 | ITIH4 | 4 | SORD | 2 | GATM | 4 |
| KCNH2 | 2 | ALDH2 | 4 | AGPAT4 | 2 | CERCAM | 4 |
| FAM134B | 2 | KLKB1 | 4 | MYO10 | 2 | CHST11 | 4 |
| SPARC | 2 | APOC1 | 4 | SLC48A1 | 2 | GPR37 | 4 |
| PRKAR1B | 2 | C1RL | 4 | EYA1 | 2 | CTNND2 | 4 |
| CLIP4 | 2 | CPB2 | 4 | SIGIRR | 2 | SH3GL3 | 4 |
| HOMER1 | 2 | PLG | 4 | CALCRL | 2 | TTYH2 | 4 |
| NDUFA4L2 | 2 | C8B | 4 | IL11RA1 | 2 | CSPG4 | 4 |
| UQCRC2 | 2 | GPD1 | 4 | KLHL7 | 2 | C1QL1 | 4 |
| UQCRC1 | 2 | C8G | 4 | PTGS1 | 2 | FNBP1 | 4 |
| IGFBP5 | 2 | AMBP | 4 | ZFAND5 | 2 | COL9A1 | 4 |
| GJA1 | 2 | SUCLG2 | 4 | CREBRF | 2 | SLAIN1 | 4 |
| MLF1 | 2 | ALDH1L1 | 4 | PPP1R16B | 2 | DSCAM | 4 |
| ALCAM | 2 | BCHE | 4 | PTOV1 | 2 | SRCIN1 | 4 |
| TGFBR3 | 2 | G6PC | 4 | CHD9 | 2 | GRB14 | 4 |
| MYH7B | 2 | SERPINF1 | 4 | TTC3 | 2 | CSRP1 | 4 |
| SLC16A3 | 2 | SERPINF2 | 4 | TTC8 | 2 | PDLIM2 | 4 |
| QK | 2 | SCP2 | 4 | DCUN1D1 | 2 | NFASC | 4 |
| PAM | 2 | TMEM56 | 3 | DYNC1LI2 | 2 | TNK2 | 4 |
| CACNA1C | 2 | ABCD3 | 3 | F11R | 2 | PLCL1 | 4 |
| CACNA1D | 2 | ORM1 | 3 | ABCG1 | 2 | TMEM151A | 4 |
| SLC25A3 | 2 | MGST2 | 3 | ABCG2 | 2 | KIF13A | 4 |
| TMEM176A | 2 | APOA5 | 3 | DEPTOR | 2 | COL11A1 | 4 |
| SRSF1 | 2 | DGAT2 | 3 | USP54 | 2 | SLC12A2 | 4 |
| PPM1K | 2 | SHROOM1 | 3 | MAPK14 | 2 | JAM3 | 4 |
| PPARGC1B | 2 | NIPSNAP1 | 3 | TEK | 2 | GRIA3 | 4 |
| TMEM71 | 2 | FAM20A | 3 | FZD6 | 2 | JOSD2 | 4 |
| PRKAB2 | 2 | FGL1 | 3 | ZFP521 | 2 | OMG | 4 |
| MSRB3 | 2 | AASS | 3 | CACNB2 | 2 | ARAP2 | 4 |
| VDAC1 | 2 | ETNK2 | 3 | SLA2 | 2 | KCND2 | 4 |
| SRL | 2 | CFH | 3 | NEURL3 | 2 | PEX5L | 4 |
| MYL12A | 2 | AGMO | 3 | MAMDC2 | 2 | GPR37L1 | 4 |
| EIF1B | 2 | SLC47A1 | 3 | GPR125 | 2 | CDH13 | 4 |
| RGS5 | 2 | DBI | 3 | TTC14 | 2 | MAP4K5 | 4 |
| RGS6 | 2 | IGFBP2 | 3 | P2RX1 | 2 | MAP4K4 | 4 |
| C530008M17RIK | 2 | CYP8B1 | 3 | BEX4 | 2 | S100B | 4 |
| PTP4A3 | 2 | GLYCTK | 3 | HOOK1 | 2 | ERBB2IP | 4 |
| BMP2 | 2 | CYP27A1 | 3 | ELTD1 | 2 | APLP1 | 4 |
| CAP2 | 2 | FETUB | 3 | KLRB1C | 2 | GSN | 4 |
| NPC2 | 2 | QPRT | 3 | RYK | 2 | ENPP4 | 4 |
| KLHL41 | 2 | ASGR1 | 3 | CTNNAL1 | 2 | CSPG5 | 4 |
| CACNB2 | 2 | PRDX6 | 3 | PEG12 | 2 | DNM3 | 4 |
| ADO | 2 | UQCRQ | 3 | DSP | 2 | SCRG1 | 4 |
| FHOD3 | 2 | GPAM | 3 | HID1 | 2 | PDCD4 | 4 |
| FAM195A | 2 | FAM46A | 3 | WDFY3 | 2 | TPRN | 3 |
| CORO6 | 2 | LYPLA1 | 3 | ACP5 | 2 | SH3D19 | 3 |
| 3425401B19RIK | 2 | HMGCL | 3 | CUEDC1 | 2 | ZFYVE16 | 3 |
| SDHD | 2 | SLC25A1 | 3 | SCARF1 | 2 | BIN1 | 3 |
| MTUS2 | 2 | TMEM176A | 3 | AK3 | 2 | CDC37L1 | 3 |
| HCN4 | 2 | TMEM176B | 3 | PRKD1 | 2 | NIPAL4 | 3 |
| KLHL31 | 2 | DNAJC22 | 3 | CTTN | 2 | PDE8A | 3 |
| DSP | 2 | HABP2 | 3 | MATK | 2 | NRXN1 | 3 |
| ACAT1 | 2 | NDRG2 | 3 | TGM2 | 2 | PAK4 | 3 |
| TOB1 | 2 | ACADM | 3 | LRRC49 | 2 | DPYSL2 | 3 |
| LRRC4B | 2 | NNMT | 3 | FCHSD2 | 2 | TRIM2 | 3 |
| ATP5B | 2 | CHCHD10 | 3 | ZFP184 | 2 | ARPP21 | 3 |
| FAM189A2 | 2 | ADI1 | 3 | MADD | 2 | NLGN2 | 3 |
| PALM2 | 2 | C9 | 3 | CYP26B1 | 2 | KCNIP1 | 3 |
| PRSS23 | 2 | TMEM205 | 3 | FZD3 | 2 | GAB1 | 3 |
| ATP5A1 | 2 | C2 | 3 | TRIM6 | 2 | MYO1D | 3 |
| FGF12 | 2 | HMGCS1 | 3 | PTPRCAP | 2 | AGPAT4 | 3 |
| HAPLN3 | 2 | GALM | 3 | SUGP2 | 2 | SLC48A1 | 3 |
| OBSCN | 2 | ALDH1A1 | 3 | CLU | 2 | CALCRL | 3 |
| PRKAA2 | 2 | GCDH | 3 | OGT | 2 | FXYD6 | 3 |
| SPHKAP | 2 | PCYOX1 | 3 | SPECC1 | 2 | BMP4 | 3 |
| LMNA | 2 | IL1RAP | 3 | H2-K1 | 2 | TRIM36 | 3 |
| UNC13C | 2 | HPD | 3 | GULP1 | 2 | PTN | 3 |
| TPI1 | 2 | PECR | 3 | SYTL4 | 2 | SCD | 3 |
| PPP1R3B | 2 | FXYD1 | 3 | GHR | 2 | RHOU | 3 |
| PPP1R3A | 2 | SLC39A14 | 3 | TBXAS1 | 2 | PADI2 | 3 |
| PARD6B | 2 | HSDL2 | 3 | FGFR3 | 2 | TPD52 | 3 |
| SPEG | 2 | ACSL5 | 3 | FGFR1 | 2 | PDZRN4 | 3 |
| GM1821 | 2 | USMG5 | 3 | SMPDL3A | 2 | PHLPP1 | 3 |
| COL12A1 | 2 | MTTP | 3 | RAI14 | 2 | FAM155A | 3 |
| NDUFA5 | 2 | RNF128 | 3 | UBA7 | 2 | TMPRSS5 | 3 |
| WNT2 | 2 | A1CF | 3 | ARHGEF6 | 2 | TMEM165 | 3 |
| PALLD | 2 | GAMT | 3 | CPXM1 | 2 | PCDH9 | 3 |
| MEST | 2 | PHYH | 3 | TMCC1 | 2 | PCDH7 | 3 |
| NDUFS6 | 2 | DECR2 | 3 | GSTM1 | 2 | PCDH11X | 3 |
| MICAL2 | 2 | DECR1 | 3 | GSTM2 | 2 | SLC45A3 | 3 |
| GOT1 | 2 | MBL2 | 3 | TREML2 | 2 | HCN2 | 3 |
| PYGM | 2 | HSD11B1 | 3 | SAT1 | 2 | ANO4 | 3 |
| HCFC1R1 | 2 | TMPRSS6 | 3 | SAMHD1 | 2 | HIP1R | 3 |
| PGM5 | 2 | PMVK | 3 | TENC1 | 2 | GRM5 | 3 |
| FYTTD1 | 2 | SDHC | 3 | TIMP3 | 2 | DST | 3 |
| USP13 | 2 | SDHB | 3 | CCDC88A | 2 | CSGALNACT1 | 3 |
| MOV10L1 | 2 | SDHD | 3 | RNASE6 | 2 | DNAJC6 | 3 |
| BRAF | 2 | SLC38A4 | 3 | KCNN4 | 2 | SLC22A23 | 3 |
| DUSP27 | 2 | SLC38A3 | 3 | ROBO4 | 2 | GRID1 | 3 |
| ACTA1 | 2 | TTC36 | 3 | INADL | 2 | RAB33A | 3 |
| CDKN1C | 2 | TTC38 | 3 | CCDC60 | 2 | SULF2 | 3 |
| APOE | 2 | ECHDC3 | 3 | MPDZ | 2 | CDK14 | 3 |
| NAB1 | 2 | ACAT1 | 3 | TNFAIP8 | 2 | SLC24A3 | 3 |
| COL2A1 | 2 | SLC27A5 | 3 | ARHGAP29 | 2 | FRYL | 3 |
| ADSSL1 | 2 | SLC17A9 | 3 | SGCE | 2 | SETD5 | 3 |
| LMOD1 | 2 | AK3 | 3 | FERMT3 | 2 | LRRTM4 | 3 |
| LMOD3 | 2 | SLC17A2 | 3 | SERPINA3G | 2 | LRRTM3 | 3 |
| LMOD2 | 2 | BAAT | 3 | GPR56 | 2 | CAR2 | 3 |
| NRP1 | 2 | DHTKD1 | 3 | CPNE8 | 2 | S1PR5 | 3 |
| FBXL22 | 2 | EHHADH | 3 | EXOC6B | 2 | TTC28 | 3 |
| MIF | 2 | DHRS1 | 3 | ICK | 2 | SLC22A17 | 3 |
| RBM24 | 2 | PCYT2 | 3 | IQCB1 | 2 | LPAR1 | 3 |
| CENPH | 2 | LEAP2 | 3 | RHBDD2 | 2 | CAR14 | 3 |
| RAMP1 | 2 | PHGDH | 3 | ACTA2 | 2 | PTK2 | 3 |
| GPI1 | 2 | HRG | 3 | EMCN | 2 | LIMS2 | 3 |
| RRAS | 2 | ECHS1 | 3 | SETBP1 | 2 | ATP10B | 3 |
| HACD1 | 2 | SMLR1 | 3 | VEGFC | 2 | NRM | 3 |
| ALDOA | 2 | RRBP1 | 3 | MYH10 | 2 | SHC3 | 3 |
| ABRACL | 2 | AGMAT | 3 | MBOAT2 | 2 | PID1 | 3 |
| CASQ1 | 2 | F10 | 3 | ATAD2B | 2 | NKAIN1 | 3 |
| TXLNB | 2 | SARDH | 3 | ARMCX1 | 2 | CCP110 | 3 |
| ADCY5 | 2 | VKORC1 | 3 | IFI44 | 2 | TALDO1 | 3 |
| ZAK | 2 | RCL1 | 3 | FUT8 | 2 | PIP4K2A | 3 |
| DCAF12L1 | 2 | CLU | 3 | EVA1B | 2 | DPP6 | 3 |
| A2M | 2 | SLC22A18 | 3 | IFITM3 | 2 | SCD5 | 3 |
| TMEM38A | 2 | KNG2 | 3 | CGNL1 | 2 | PRUNE2 | 3 |
| PTGES3L | 2 | SERPINA1C | 3 | PABPC4L | 2 | SCN3A | 3 |
| KLHL30 | 2 | DNAJC12 | 3 | KMT2D | 2 | ELOVL7 | 3 |
| GBAS | 2 | FMO5 | 3 | PLA2G16 | 2 | ELOVL1 | 3 |
| RBM20 | 2 | IDH1 | 3 | TRPC6 | 2 | NRBP2 | 3 |
| ME2 | 2 | RGN | 3 | VGLL4 | 2 | ATP8A1 | 3 |
| CITED1 | 2 | PNPLA3 | 3 | FGD5 | 2 | RDX | 3 |
| VCAM1 | 2 | ECH1 | 3 | PARD3B | 2 | PRR5L | 3 |
| PDE4DIP | 2 | GULO | 3 | LPHN2 | 2 | FAM83D | 3 |
| SNTA1 | 2 | SEC14L2 | 3 | CARD10 | 2 | PXK | 3 |
| TUBA1A | 2 | NDUFA4 | 3 | NCEH1 | 2 | DGKB | 3 |
| FKBP3 | 2 | CYP1A2 | 3 | MMP2 | 2 | ZDHHC14 | 3 |
| GPX3 | 2 | AGXT | 3 | BCL2L1 | 2 | OTUD7B | 3 |
| FLNC | 2 | CBR1 | 3 | PODXL | 2 | ARHGEF10 | 3 |
| CRIP1 | 2 | SDS | 3 | IL12RB2 | 2 | SLITRK2 | 3 |
| KRT19 | 2 | HIBADH | 3 | ENSA | 2 | CNDP1 | 3 |
| TMEM88 | 2 | SOD1 | 3 | AQP1 | 2 | GALNT13 | 3 |
| RCAN2 | 2 | NIT2 | 3 | FAM132A | 2 | SGK1 | 3 |
| TRIM54 | 2 | SLC7A2 | 3 | RBBP9 | 2 | EDNRB | 3 |
| LAMA4 | 2 | KRT8 | 3 | ADCY7 | 2 | FBXO32 | 3 |
| MYBPHL | 2 | PGRMC1 | 3 | PNPO | 2 | S100A1 | 3 |
| MUM1L1 | 2 | SDC2 | 3 | GUCY1B3 | 2 | TMEM63A | 3 |
| DKK3 | 2 | GCH1 | 3 | DOCK1 | 2 | EMID1 | 3 |
| PKP2 | 2 | ECI2 | 3 | DOCK6 | 2 | TRIM9 | 3 |
| LBR | 2 | ECI1 | 3 | ZFP788 | 2 | KAZN | 3 |
| LBH | 2 | SAA4 | 3 | GIMAP8 | 2 | PDE1C | 3 |
| ATP1B1 | 2 | ACAD11 | 3 | GIMAP6 | 2 | ANK2 | 3 |
| VIM | 2 | PEMT | 3 | PHF6 | 2 | ADAMTS4 | 3 |
| YPEL2 | 2 | SEMA4G | 3 | TIE1 | 2 | LDB3 | 3 |
| SMYD2 | 2 | TAT | 3 | MDFI | 2 | REV3L | 3 |
| FAM78A | 2 | ANGPTL4 | 3 | LAX1 | 2 | SHISA9 | 3 |
| FILIP1 | 2 | GLS2 | 3 | FHL1 | 2 | ASTN1 | 3 |
| GAPDH | 2 | CAT | 3 | YES1 | 2 | SEMA5A | 3 |
| USP2 | 2 | GHR | 3 | FSTL1 | 2 | CADM2 | 3 |
| NDRG2 | 2 | ATP5G3 | 3 | ALOX5 | 2 | CADM1 | 3 |
| LOXL2 | 2 | STARD10 | 3 | KLHL4 | 2 | RALGDS | 3 |
| PFKP | 2 | GOS2 | 3 | MAGED1 | 2 | CDC42EP2 | 3 |
| CTNNA3 | 2 | MTHFD1 | 3 | ZFP12 | 2 | TMEM144 | 3 |
| CNN2 | 2 | CPN2 | 3 | PCMTD1 | 2 | FMNL2 | 3 |
| PGK1 | 2 | CPN1 | 3 | CMAS | 2 | PLD1 | 3 |
| SMTNL2 | 2 | CALD1 | 3 | CLDN12 | 2 | PPFIBP1 | 3 |
| RBFOX2 | 2 | ACADVL | 3 | HNRNPH3 | 2 | GRM3 | 3 |
| ATP5G3 | 2 | TFR2 | 3 | MPZL1 | 2 | SOX2-OT | 3 |
| HSPB3 | 2 | DDT | 3 | HSPA12B | 2 | GRIA2 | 3 |
| HSPB1 | 2 | ANG | 3 | CITED2 | 2 | GRIA4 | 3 |
| ALDOC | 2 | ALDH4A1 | 3 | CC2D2A | 2 | LIMCH1 | 3 |
| CACNA2D2 | 2 | A2M | 3 | UNC13D | 2 | PLEKHB1 | 3 |
| CYP2S1 | 2 | SLC10A1 | 3 | VAMP5 | 2 | PLCB1 | 3 |
| PLPPR5 | 2 | DCXR | 3 | CNTLN | 2 | EPHB1 | 3 |
| GYS1 | 2 | PCBD1 | 3 | OSGIN1 | 2 | MOB3B | 3 |
| CFL2 | 2 | ALDH7A1 | 3 | SLC9A3R2 | 2 | SUSD5 | 3 |
| | | RARRES2 | 3 | PREX2 | 2 | FTH1 | 3 |
| | | OAF | 3 | PTPRE | 2 | SLCO3A1 | 3 |
| | | OAT | 3 | MFAP2 | 2 | CHADL | 3 |
| | | ENTPD5 | 3 | PIK3R1 | 2 | TMBIM1 | 3 |
| | | CPS1 | 3 | GRB10 | 2 | CLMN | 3 |
| | | TDO2 | 3 | CCND2 | 2 | KLHL5 | 3 |
| | | GLYAT | 3 | LAT | 2 | KLHL2 | 3 |
| | | CMBL | 3 | RC3H2 | 2 | CYP2J12 | 3 |
| | | LECT2 | 3 | PDLIM1 | 2 | PCBP4 | 3 |
| | | RPS27L | 3 | ZDHHC1 | 2 | SPOCK3 | 3 |
| | | INSIG1 | 3 | CYP2J6 | 2 | SEMA3D | 3 |
| | | NDUFV2 | 3 | NBEAL2 | 2 | RIN2 | 3 |
| | | ALDH9A1 | 3 | GM13152 | 2 | RNF144A | 3 |
| | | F5 | 3 | MUM1L1 | 2 | FRMD4A | 3 |
| | | F9 | 3 | IER3 | 2 | KCNJ10 | 3 |
| | | ABCA6 | 3 | WBSCR27 | 2 | PTPRG | 3 |
| | | TM4SF4 | 3 | DDAH1 | 2 | PTPRE | 3 |
| | | APCS | 3 | DDAH2 | 2 | GPM6A | 3 |
| | | HSD17B4 | 3 | VSIG2 | 2 | CCND2 | 3 |
| | | MPC2 | 3 | YPEL3 | 2 | CHL1 | 3 |
| | | LBP | 3 | TRAF3IP3 | 2 | C1ORF61 | 3 |
| | | HSD17B2 | 3 | PLXDC2 | 2 | TRIM59 | 3 |
| | | DDAH1 | 3 | ORAI1 | 2 | SDC3 | 3 |
| | | CFHR1 | 3 | JAM3 | 2 | BAALC | 3 |
| | | ETFB | 3 | CD74 | 2 | SPON1 | 3 |
| | | LCAT | 3 | CHAD | 2 | RTN4 | 3 |
| | | ABCB4 | 3 | CIRBP | 2 | PARD3 | 3 |
| | | HDLBP | 3 | CBX6 | 2 | PKP4 | 3 |
| | | CIDEB | 3 | DOCK2 | 2 | XYLT1 | 3 |
| | | DHCR24 | 3 | KLHL13 | 2 | TNFRSF21 | 3 |
| | | TM7SF2 | 3 | AMIGO2 | 2 | D16ERTD472E | 3 |
| | | APOC3 | 3 | GABARAPL2 | 2 | GNG7 | 3 |
| | | ASPDH | 3 | UNC119 | 2 | TTLL7 | 3 |
| | | ADK | 3 | VIPR2 | 2 | YPEL2 | 3 |
| | | SELENBP1 | 3 | TTPA | 2 | GALNT6 | 3 |
| | | ETFDH | 3 | SELENBP1 | 2 | EFNB3 | 3 |
| | | CRP | 3 | FAM110C | 2 | FABP7 | 3 |
| | | GLDC | 3 | GBP7 | 2 | KAT2B | 3 |
| | | SERPING1 | 3 | CHRNB1 | 2 | MYO6 | 3 |
| | | IYD | 3 | GIMAP1 | 2 | NDRG1 | 3 |
| | | C8A | 3 | EFNA1 | 2 | TPPP | 3 |
| | | COL18A1 | 3 | OCLN | 2 | DHCR24 | 3 |
| | | EBPL | 3 | EFCAB14 | 2 | MAP6D1 | 3 |
| | | PSAT1 | 3 | CREG1 | 2 | CERS2 | 3 |
| | | HAO1 | 3 | PKN1 | 2 | SNTG1 | 3 |
| | | ABCC6 | 3 | RHOBTB3 | 2 | MAML2 | 3 |
| | | ABCC2 | 3 | FAM213A | 2 | MAN2A1 | 3 |
| | | QDPR | 3 | TROVE2 | 2 | CD9 | 3 |
| | | UGP2 | 3 | ITSN1 | 2 | CD82 | 3 |
| | | FDPS | 3 | HK1 | 2 | EFCAB14 | 3 |
| | | PTMS | 3 | DKKL1 | 2 | LRP1 | 3 |
| | | H6PD | 3 | DNMT3B | 2 | PDE4B | 3 |
| | | GLUL | 3 | UBE2L6 | 2 | SEPP1 | 3 |
| | | ALDH5A1 | 3 | SLC25A36 | 2 | MAP7 | 3 |
| | | ARG1 | 3 | NBEA | 2 | RAP1GAP | 3 |
| | | GCSH | 3 | EHD3 | 2 | MT3 | 3 |
| | | ALAD | 3 | STAU2 | 2 | LDLRAD3 | 3 |
| | | GLUD1 | 3 | LEPREL2 | 2 | NAV1 | 3 |
| | | LDHA | 3 | RAB37 | 2 | NAV2 | 3 |
| | | LONP2 | 3 | NDN | 2 | LRRC4C | 3 |
| | | LRG1 | 3 | LDHB | 2 | KCTD13 | 3 |
| | | AGL | 2 | YPEL2 | 2 | MEGF11 | 3 |
| | | HRSP12 | 2 | PTRF | 2 | AFAP1L2 | 3 |
| | | HSPA9 | 2 | | | IGSF11 | 3 |
| | | SPR | 2 | | | NCAM2 | 3 |
| | | ZC3H13 | 2 | | | RNF220 | 3 |
| | | GOLIM4 | 2 | | | RAB37 | 3 |
| | | CLDN3 | 2 | | | CLASP2 | 3 |
| | | ORM2 | 2 | | | CFL2 | 3 |
| | | ACLY | 2 | | | SPATA6 | 3 |
| | | PNISR | 2 | | | RNF13 | 2 |
| | | PHLDA1 | 2 | | | LINC00609 | 2 |
| | | LSR | 2 | | | CRB1 | 2 |
| | | LSS | 2 | | | CNP1 | 2 |
| | | TMEM192 | 2 | | | MTMR10 | 2 |
| | | SIGIRR | 2 | | | AI414108 | 2 |
| | | FAM134B | 2 | | | LSS | 2 |
| | | FAHD1 | 2 | | | COL4A5 | 2 |
| | | AIG1 | 2 | | | CHST3 | 2 |
| | | PRKAR1A | 2 | | | KCNH8 | 2 |
| | | SAR1B | 2 | | | ITGAD | 2 |
| | | SLCO1B1 | 2 | | | TUBB2B | 2 |
| | | SLCO1B3 | 2 | | | DIP2C | 2 |
| | | CYP3A13 | 2 | | | GLTP | 2 |
| | | CHP1 | 2 | | | ENOPH1 | 2 |
| | | PGLYRP2 | 2 | | | NOL4 | 2 |
| | | GCHFR | 2 | | | SRPK3 | 2 |
| | | DPYD | 2 | | | CADPS2 | 2 |
| | | RDH7 | 2 | | | ADARB2 | 2 |
| | | PEX14 | 2 | | | SPP1 | 2 |
| | | FPGS | 2 | | | CTHRC1 | 2 |
| | | CYB5A | 2 | | | RGS20 | 2 |
| | | CDO1 | 2 | | | DPYD | 2 |
| | | APOC4-APOC2 | 2 | | | PCDH17 | 2 |
| | | CHI3L1 | 2 | | | CA10 | 2 |
| | | CYP4F2 | 2 | | | SASH1 | 2 |
| | | CYP4F3 | 2 | | | FAR1 | 2 |
| | | MRPL34 | 2 | | | CDO1 | 2 |
| | | CSAD | 2 | | | SORCS1 | 2 |
| | | UQCRFS1 | 2 | | | ARRDC3 | 2 |
| | | UQCRC1 | 2 | | | DBI | 2 |
| | | GGCX | 2 | | | APP | 2 |
| | | RETSAT | 2 | | | SERINC5 | 2 |
| | | SAA2 | 2 | | | KHDRBS3 | 2 |
| | | PINK1 | 2 | | | MDGA2 | 2 |
| | | CYP2D6 | 2 | | | PEAK1 | 2 |
| | | AKR1C1 | 2 | | | MFSD2A | 2 |
| | | FDFT1 | 2 | | | PRKG2 | 2 |
| | | IGF2 | 2 | | | FDFT1 | 2 |
| | | IGFBP4 | 2 | | | C1ORF21 | 2 |
| | | AFP | 2 | | | COL16A1 | 2 |
| | | COX17 | 2 | | | ABTB2 | 2 |
| | | UGT2A3 | 2 | | | GAS1 | 2 |
| | | PPA1 | 2 | | | AMPD3 | 2 |
| | | CFHR3 | 2 | | | CSMD2 | 2 |
| | | PLPP5 | 2 | | | CSMD3 | 2 |
| | | CFHR4 | 2 | | | CDKN1C | 2 |
| | | UGDH | 2 | | | PRIMA1 | 2 |
| | | TJP2 | 2 | | | GPCPD1 | 2 |
| | | PRG4 | 2 | | | TRIL | 2 |
| | | SH3PXD2A | 2 | | | NLGN3 | 2 |
| | | PRDX4 | 2 | | | UGDH | 2 |
| | | PRDX3 | 2 | | | AC012593.1 | 2 |
| | | PRDX1 | 2 | | | TMEM141 | 2 |
| | | SMOC1 | 2 | | | TJP1 | 2 |
| | | POR | 2 | | | RTKN2 | 2 |
| | | PPP1R1A | 2 | | | MPPED2 | 2 |
| | | NSUN6 | 2 | | | PRDX1 | 2 |
| | | DCAF6 | 2 | | | PDK4 | 2 |
| | | ACADSB | 2 | | | SBDS | 2 |
| | | A1BG | 2 | | | GM98 | 2 |
| | | SLC25A4 | 2 | | | ARHGAP31 | 2 |
| | | HMGCR | 2 | | | LAMP2 | 2 |
| | | ORMDL3 | 2 | | | B3GAT1 | 2 |
| | | PBLD2 | 2 | | | CACNA1A | 2 |
| | | KIF5B | 2 | | | DCC | 2 |
| | | MASP1 | 2 | | | DLGAP1 | 2 |
| | | SLC31A1 | 2 | | | TMEM178B | 2 |
| | | C4BPA | 2 | | | WSB1 | 2 |
| | | C4BPB | 2 | | | CDH20 | 2 |
| | | XDH | 2 | | | TMEM176B | 2 |
| | | CISD3 | 2 | | | SLC15A4 | 2 |
| | | CISD1 | 2 | | | CCDC50 | 2 |
| | | PTPN3 | 2 | | | RRAS2 | 2 |
| | | ATOX1 | 2 | | | NDRG2 | 2 |
| | | KMO | 2 | | | RNF130 | 2 |
| | | MSRB1 | 2 | | | S100A16 | 2 |
| | | C6 | 2 | | | DDR1 | 2 |
| | | C5 | 2 | | | C10ORF90 | 2 |
| | | LGALS9 | 2 | | | NKX6.2 | 2 |
| | | LGALS4 | 2 | | | MYO1E | 2 |
| | | CRYZ | 2 | | | RTKN | 2 |
| | | SPINK1 | 2 | | | ADI1 | 2 |
| | | AADAT | 2 | | | TPPP3 | 2 |
| | | HPR | 2 | | | LY6G6F | 2 |
| | | C1RA | 2 | | | HMGCS1 | 2 |
| | | RDH16 | 2 | | | ARHGAP24 | 2 |
| | | CYP2C70 | 2 | | | IGSF21 | 2 |
| | | PLIN5 | 2 | | | ASAP1 | 2 |
| | | PLIN2 | 2 | | | CMYA5 | 2 |
| | | PTP4A1 | 2 | | | TMEM159 | 2 |
| | | RBP5 | 2 | | | PRICKLE1 | 2 |
| | | ABHD2 | 2 | | | HIP1 | 2 |
| | | MT1M | 2 | | | D7ERTD443E | 2 |
| | | UBR4 | 2 | | | 7-Sep | 2 |
| | | MLYCD | 2 | | | PSEN1 | 2 |
| | | HC | 2 | | | EPCAM | 2 |
| | | ATP5C1 | 2 | | | PIGZ | 2 |
| | | SCD | 2 | | | FSD1L | 2 |
| | | AKR1C4 | 2 | | | RHPN2 | 2 |
| | | GADD45B | 2 | | | WFDC18 | 2 |
| | | COL27A1 | 2 | | | NTRK2 | 2 |
| | | MRPL12 | 2 | | | ARHGAP20 | 2 |
| | | ITPR2 | 2 | | | DDIT4 | 2 |
| | | CHDH | 2 | | | CD22 | 2 |
| | | AKR7A3 | 2 | | | KCNMB4 | 2 |
| | | IL1RN | 2 | | | ARPC1B | 2 |
| | | IL17RC | 2 | | | ACSL1 | 2 |
| | | CD302 | 2 | | | CCNY | 2 |
| | | HPGD | 2 | | | LRP4 | 2 |
| | | UGT2B15 | 2 | | | KCNQ1OT1 | 2 |
| | | UGT2B10 | 2 | | | NCKAP5 | 2 |
| | | SRD5A1 | 2 | | | LRRK2 | 2 |
| | | ADH1 | 2 | | | CHD9 | 2 |
| | | ADH6 | 2 | | | HTRA1 | 2 |
| | | ADH5 | 2 | | | MBP6 | 2 |
| | | ADH4 | 2 | | | TANC2 | 2 |
| | | IGFALS | 2 | | | RHOG | 2 |
| | | PSMB7 | 2 | | | ANKRD28 | 2 |
| | | MBL1 | 2 | | | RNF128 | 2 |
| | | HADHA | 2 | | | RNF122 | 2 |
| | | HIGD1A | 2 | | | DSCAML1 | 2 |
| | | ACOT12 | 2 | | | GAMT | 2 |
| | | KLB | 2 | | | NLGN4X | 2 |
| | | RMDN1 | 2 | | | FZD9 | 2 |
| | | UROC1 | 2 | | | CTNNA3 | 2 |
| | | NADK2 | 2 | | | ADO | 2 |
| | | CYP2C68 | 2 | | | LGR5 | 2 |
| | | SLC38A2 | 2 | | | TMEM163 | 2 |
| | | PTGR1 | 2 | | | EVI2A | 2 |
| | | CYP3A4 | 2 | | | PPAP2C | 2 |
| | | STARD4 | 2 | | | CTTNBP2 | 2 |
| | | CELF1 | 2 | | | ANKIB1 | 2 |
| | | PEPD | 2 | | | DLG1 | 2 |
| | | TMEM97 | 2 | | | SAMD12 | 2 |
| | | GSTO1 | 2 | | | LINC01608 | 2 |
| | | GLYATL1 | 2 | | | AKAP6 | 2 |
| | | ECHDC1 | 2 | | | TMEM132C | 2 |
| | | PRODH2 | 2 | | | ATP1A2 | 2 |
| | | RHOB | 2 | | | CAMSAP2 | 2 |
| | | ACAT2 | 2 | | | ERMP1 | 2 |
| | | ACAT3 | 2 | | | WDFY2 | 2 |
| | | RNF152 | 2 | | | MKRN3 | 2 |
| | | ELL2 | 2 | | | LHFPL2 | 2 |
| | | LDLR | 2 | | | PHYHIPL | 2 |
| | | CYP2C9 | 2 | | | ACAT2 | 2 |
| | | CYP2C8 | 2 | | | FAM110B | 2 |
| | | ZFYVE21 | 2 | | | KIF21A | 2 |
| | | AK4 | 2 | | | GRID2 | 2 |
| | | HSPE1 | 2 | | | ZNF638 | 2 |
| | | ERBB3 | 2 | | | TBCB | 2 |
| | | LARP1B | 2 | | | AK5 | 2 |
| | | HERPUD1 | 2 | | | PRKD1 | 2 |
| | | TMEM220 | 2 | | | ERC1 | 2 |
| | | LACTB2 | 2 | | | EPS8 | 2 |
| | | RND3 | 2 | | | GRIN3A | 2 |
| | | FUOM | 2 | | | NEO1 | 2 |
| | | IDI1 | 2 | | | SHROOM4 | 2 |
| | | MTCH2 | 2 | | | VWC2 | 2 |
| | | SLC2A2 | 2 | | | NLGN4Y | 2 |
| | | F11 | 2 | | | BFSP2 | 2 |
| | | 2-Mar | 2 | | | RGCC | 2 |
| | | SERPINA11 | 2 | | | SLC1A1 | 2 |
| | | PSMB10 | 2 | | | 1-Mar | 2 |
| | | CYP2B6 | 2 | | | DAAM2 | 2 |
| | | ATP5A1 | 2 | | | PCYT2 | 2 |
| | | HFE2 | 2 | | | FCHSD2 | 2 |
| | | KEG1 | 2 | | | HEPN1 | 2 |
| | | HNRNPA3 | 2 | | | DIXDC1 | 2 |
| | | IL6R | 2 | | | LINC01322 | 2 |
| | | IRS2 | 2 | | | IL33 | 2 |
| | | SERPINA1B | 2 | | | CAR8 | 2 |
| | | SERPINA1A | 2 | | | TAOK3 | 2 |
| | | SERPINA1D | 2 | | | GRIK2 | 2 |
| | | SERPINA1E | 2 | | | SPECC1 | 2 |
| | | PHB2 | 2 | | | SEMA6A | 2 |
| | | SF3B1 | 2 | | | GYG | 2 |
| | | SORBS3 | 2 | | | SUN2 | 2 |
| | | HSPD1 | 2 | | | PRKCA | 2 |
| | | UGT2B36 | 2 | | | BCAS11 | 2 |
| | | UGT2B34 | 2 | | | SEC11C | 2 |
| | | UGT2B35 | 2 | | | GSTP1 | 2 |
| | | CES1 | 2 | | | PKD2L1 | 2 |
| | | CES2 | 2 | | | IDH1 | 2 |
| | | LMAN1 | 2 | | | NACAD | 2 |
| | | FAM195A | 2 | | | TMEM100 | 2 |
| | | GSTP1 | 2 | | | FGFR2 | 2 |
| | | PCK2 | 2 | | | PLP19 | 2 |
| | | FKBP11 | 2 | | | VPS37B | 2 |
| | | MARVELD2 | 2 | | | GRIK1 | 2 |
| | | ZNF207 | 2 | | | TIAM1 | 2 |
| | | IDH2 | 2 | | | NKAIN3 | 2 |
| | | GSTA3 | 2 | | | MAP1B | 2 |
| | | GPX4 | 2 | | | UNC5B | 2 |
| | | THRSP | 2 | | | UNC5C | 2 |
| | | PHKA2 | 2 | | | FRMD8 | 2 |
| | | FAM162A | 2 | | | FRMD5 | 2 |
| | | CDHR5 | 2 | | | BTBD16 | 2 |
| | | FGGY | 2 | | | SIK3 | 2 |
| | | SEC14L4 | 2 | | | TMCC3 | 2 |
| | | SIK2 | 2 | | | CYP51 | 2 |
| | | NDUFA1 | 2 | | | GPR62 | 2 |
| | | COMT | 2 | | | WNT3 | 2 |
| | | FIS1 | 2 | | | LRRN1 | 2 |
| | | EFHD1 | 2 | | | IL23A | 2 |
| | | INSR | 2 | | | TIMP4 | 2 |
| | | GNE | 2 | | | ARRDC2 | 2 |
| | | ANKRD17 | 2 | | | BOK | 2 |
| | | TMEM37 | 2 | | | SUSD4 | 2 |
| | | CPT1A | 2 | | | RNASE1 | 2 |
| | | PAPSS2 | 2 | | | BRINP1 | 2 |
| | | C1S | 2 | | | STXBP3A | 2 |
| | | C1R | 2 | | | STMN4 | 2 |
| | | AKR1C13 | 2 | | | AMER2 | 2 |
| | | CPT2 | 2 | | | ZFP276 | 2 |
| | | GOT1 | 2 | | | ZCCHC24 | 2 |
| | | PYGL | 2 | | | 42986 | 2 |
| | | SOD2 | 2 | | | TNFAIP6 | 2 |
| | | RNASE4 | 2 | | | ELOVL6 | 2 |
| | | TF | 2 | | | PDZD2 | 2 |
| | | SMIM24 | 2 | | | SORCS3 | 2 |
| | | FST | 2 | | | SGCD | 2 |
| | | SLC19A2 | 2 | | | CNKSR3 | 2 |
| | | NACA | 2 | | | GP1BB | 2 |
| | | PGM1 | 2 | | | ANKS1B | 2 |
| | | PGM2 | 2 | | | AUTS2 | 2 |
| | | TRF | 2 | | | UST | 2 |
| | | SDC1 | 2 | | | GPR56 | 2 |
| | | ELOVL5 | 2 | | | EXOC6B | 2 |
| | | CYP2C44 | 2 | | | CPNE2 | 2 |
| | | TECR | 2 | | | RFTN2 | 2 |
| | | PAQR9 | 2 | | | RFTN1 | 2 |
| | | PHYHD1 | 2 | | | DGKG | 2 |
| | | RDX | 2 | | | ICK | 2 |
| | | FERMT2 | 2 | | | TMCC2 | 2 |
| | | FADS1 | 2 | | | JPH1 | 2 |
| | | CCL16 | 2 | | | OTUD7A | 2 |
| | | SERPINA1 | 2 | | | NCAN | 2 |
| | | SERPINA4 | 2 | | | AMZ1 | 2 |
| | | SERPINA5 | 2 | | | ATRNL1 | 2 |
| | | SERPINA7 | 2 | | | SCCPDH | 2 |
| | | HAGH | 2 | | | RASGRP3 | 2 |
| | | COPZ1 | 2 | | | GPM6B5 | 2 |
| | | PCSK9 | 2 | | | D630045J12RIK | 2 |
| | | ESPN | 2 | | | TMEM123 | 2 |
| | | H2-Q10 | 2 | | | DLG2 | 2 |
| | | SCCPDH | 2 | | | PPP1R18 | 2 |
| | | CNDP1 | 2 | | | LMF1 | 2 |
| | | CDA | 2 | | | INSC | 2 |
| | | PTER | 2 | | | ADSSL1 | 2 |
| | | GADD45GIP1 | 2 | | | STK32A | 2 |
| | | FDX1 | 2 | | | ARL8A | 2 |
| | | ANGPTL8 | 2 | | | PDE1A | 2 |
| | | GPLD1 | 2 | | | PCDH20 | 2 |
| | | APOE | 2 | | | 3110035E14RIK | 2 |
| | | HNMT | 2 | | | ARSG | 2 |
| | | NUPR1 | 2 | | | ARSB | 2 |
| | | VEGFA | 2 | | | ANK3 | 2 |
| | | SLC39A4 | 2 | | | FUT9 | 2 |
| | | SLC39A5 | 2 | | | FUT8 | 2 |
| | | FBXO31 | 2 | | | RLBP1 | 2 |
| | | AOX1 | 2 | | | DAB1 | 2 |
| | | METTL7A1 | 2 | | | USP24 | 2 |
| | | HINT1 | 2 | | | DPP10 | 2 |
| | | HINT2 | 2 | | | SCD3 | 2 |
| | | ALAS1 | 2 | | | ENOX1 | 2 |
| | | LEPR | 2 | | | RAMP1 | 2 |
| | | RIOK3 | 2 | | | SSH3 | 2 |
| | | ZNHIT1 | 2 | | | SCARB2 | 2 |
| | | EGFR | 2 | | | CSMD1 | 2 |
| | | NDUFC2 | 2 | | | G0S2 | 2 |
| | | LGALS3BP | 2 | | | ASTN2 | 2 |
| | | IL32 | 2 | | | HHATL | 2 |
| | | CA2 | 2 | | | PLA2G16 | 2 |
| | | EPHX1 | 2 | | | SEMA5B | 2 |
| | | GABPB1-AS1 | 2 | | | HS6ST3 | 2 |
| | | GSTA1 | 2 | | | DUSP15 | 2 |
| | | GSTA2 | 2 | | | GM10863 | 2 |
| | | AGXT2 | 2 | | | RASSF10 | 2 |
| | | CDH1 | 2 | | | CADM4 | 2 |
| | | CDH2 | 2 | | | FGD3 | 2 |
| | | IFITM3 | 2 | | | UNC80 | 2 |
| | | IFITM2 | 2 | | | FAM107B | 2 |
| | | AMACR | 2 | | | CLIC4 | 2 |
| | | SCARB1 | 2 | | | TMEM132B | 2 |
| | | ADGRG6 | 2 | | | AGAP1 | 2 |
| | | HULC | 2 | | | FEZ1 | 2 |
| | | PROS1 | 2 | | | SLCO1A2 | 2 |
| | | ARFGAP2 | 2 | | | GM9895 | 2 |
| | | TRIB1 | 2 | | | MAPRE2 | 2 |
| | | ARHGEF10L | 2 | | | CRYL1 | 2 |
| | | MT1X | 2 | | | GRM7 | 2 |
| | | GATM | 2 | | | LIMA1 | 2 |
| | | MT1H | 2 | | | DOCK1 | 2 |
| | | MT1E | 2 | | | SEPT4 | 2 |
| | | MT1F | 2 | | | SLC9A9 | 2 |
| | | MT1G | 2 | | | DDC | 2 |
| | | MT1A | 2 | | | AAMDC | 2 |
| | | COX6C | 2 | | | CCDC88A | 2 |
| | | VNN1 | 2 | | | FBXO7 | 2 |
| | | SLC23A2 | 2 | | | KCNA1 | 2 |
| | | CFHR2 | 2 | | | MTSS1L | 2 |
| | | CHST13 | 2 | | | GAREML | 2 |
| | | CYP2C18 | 2 | | | IL1RAPL1 | 2 |
| | | NDUFB9 | 2 | | | DIP2B | 2 |
| | | PCCB | 2 | | | PTPRZ12 | 2 |
| | | PZP | 2 | | | FAM19A2 | 2 |
| | | SLC22A7 | 2 | | | FAH | 2 |
| | | CRYL1 | 2 | | | HNRNPA2B1 | 2 |
| | | ACSM2A | 2 | | | SYNJ2 | 2 |
| | | AQP9 | 2 | | | MIDN | 2 |
| | | ACOT4 | 2 | | | SHTN1 | 2 |
| | | SHMT2 | 2 | | | NKX2-9 | 2 |
| | | TOR1AIP2 | 2 | | | ATP2B4 | 2 |
| | | ADCY1 | 2 | | | ELFN2 | 2 |
| | | DDI2 | 2 | | | TMEM132D | 2 |
| | | RBM39 | 2 | | | DLEU2 | 2 |
| | | DST | 2 | | | FBXL7 | 2 |
| | | DDC | 2 | | | PLXNB3 | 2 |
| | | GSTT1 | 2 | | | GRAMD3 | 2 |
| | | PHF8 | 2 | | | KLHL32 | 2 |
| | | PPP2R5A | 2 | | | SEZ6L2 | 2 |
| | | NADK | 2 | | | C9ORF3 | 2 |
| | | SMIM14 | 2 | | | SPOCK1 | 2 |
| | | NAMPT | 2 | | | CLDN14 | 2 |
| | | ACACB | 2 | | | MPZL1 | 2 |
| | | PLA2G2A | 2 | | | FGF12 | 2 |
| | | ATRX | 2 | | | FGF14 | 2 |
| | | COX7B | 2 | | | COL9A3 | 2 |
| | | HIBCH | 2 | | | UNC13C | 2 |
| | | MDH1 | 2 | | | SYNE2 | 2 |
| | | HNRNPA2B1 | 2 | | | DEGS1 | 2 |
| | | ZCCHC6 | 2 | | | CNTNAP5 | 2 |
| | | DIO1 | 2 | | | CNTNAP4 | 2 |
| | | TMBIM6 | 2 | | | PAPSS1 | 2 |
| | | ENPP1 | 2 | | | TF | 2 |
| | | CCS | 2 | | | PTPRT | 2 |
| | | RALGAPA2 | 2 | | | PTPRD | 2 |
| | | P4HB | 2 | | | PTPRO | 2 |
| | | SPRYD4 | 2 | | | PTPRK | 2 |
| | | FAXDC2 | 2 | | | PTPRJ | 2 |
| | | CDV3 | 2 | | | ADGRL3 | 2 |
| | | LINC00844 | 2 | | | CPM | 2 |
| | | TKFC | 2 | | | UBL3 | 2 |
| | | ADGRA3 | 2 | | | THBS3 | 2 |
| | | ATP5J | 2 | | | GPM6B | 2 |
| | | TENM1 | 2 | | | APBB1 | 2 |
| | | ATP5H | 2 | | | GPRC5B | 2 |
| | | ATP50 | 2 | | | ABLIM2 | 2 |
| | | ATP5D | 2 | | | CCND1 | 2 |
| | | AFMID | 2 | | | KCNMA1 | 2 |
| | | OSGIN1 | 2 | | | ZDHHC9 | 2 |
| | | PDE4DIP | 2 | | | CYP2J9 | 2 |
| | | ARHGEF26 | 2 | | | PARVB | 2 |
| | | DESI1 | 2 | | | IL12RB1 | 2 |
| | | TXN | 2 | | | GM6682 | 2 |
| | | UBE2G2 | 2 | | | FAM89A | 2 |
| | | CYP4A11 | 2 | | | SPARCL1 | 2 |
| | | ACTN4 | 2 | | | EPS15 | 2 |
| | | CYP3A5 | 2 | | | QKI | 2 |
| | | FKBP4 | 2 | | | HSD17B7 | 2 |
| | | FKBP5 | 2 | | | LRRC7 | 2 |
| | | SRSF11 | 2 | | | ITGB4 | 2 |
| | | TIMD2 | 2 | | | BACE1 | 2 |
| | | ABLIM3 | 2 | | | SLC35F3 | 2 |
| | | GRB14 | 2 | | | B230206H07RIK | 2 |
| | | AK2 | 2 | | | KIF13B | 2 |
| | | BBOX1 | 2 | | | CMTM5 | 2 |
| | | SFXN1 | 2 | | | MPC1 | 2 |
| | | SERPINE1 | 2 | | | TSPAN12 | 2 |
| | | CCT8 | 2 | | | TSPAN15 | 2 |
| | | C4B | 2 | | | KIRREL3 | 2 |
| | | HEATR5A | 2 | | | TESK2 | 2 |
| | | INSIG2 | 2 | | | GNG8 | 2 |
| | | CYP2D26 | 2 | | | H2-AB1 | 2 |
| | | ZDHHC9 | 2 | | | PPFIBP2 | 2 |
| | | GPX1 | 2 | | | SAPCD2 | 2 |
| | | CBS | 2 | | | FIGN | 2 |
| | | GPX3 | 2 | | | LINC00511 | 2 |
| | | GPX2 | 2 | | | CLK1 | 2 |
| | | ANPEP | 2 | | | SH3BP4 | 2 |
| | | TTC39C | 2 | | | FABP5 | 2 |
| | | RCAN1 | 2 | | | PANX1 | 2 |
| | | ACSM5 | 2 | | | LINC00461 | 2 |
| | | CROT | 2 | | | DOCK4 | 2 |
| | | XIAP | 2 | | | LAMP1 | 2 |
| | | FH | 2 | | | REEP3 | 2 |
| | | PSMD9 | 2 | | | DLC1 | 2 |
| | | NDUFV3 | 2 | | | TNNI1 | 2 |
| | | NDUFV1 | 2 | | | SCN1A | 2 |
| | | ASPG | 2 | | | CTNNA2 | 2 |
| | | GGH | 2 | | | FAM181B | 2 |
| | | FAM96A | 2 | | | ARHGAP21 | 2 |
| | | MVD | 2 | | | TM7SF3 | 2 |
| | | BPHL | 2 | | | RAPGEF5 | 2 |
| | | MVK | 2 | | | MPP5 | 2 |
| | | C11ORF54 | 2 | | | INHBB | 2 |
| | | MT2A | 2 | | | MAGI2 | 2 |
| | | COX5B | 2 | | | DEB1 | 2 |
| | | EI24 | 2 | | | MYLK | 2 |
| | | RTP3 | 2 | | | FAM13C | 2 |
| | | TM4SF5 | 2 | | | CORO2B | 2 |
| | | ACAA1 | 2 | | | STRN | 2 |
| | | HSD17B6 | 2 | | | KNOP1 | 2 |
| | | MPC1 | 2 | | | STK39 | 2 |
| | | LINC01554 | 2 | | | CD81 | 2 |
| | | SLC25A18 | 2 | | | ANKRD36C | 2 |
| | | SLC25A15 | 2 | | | RDX1 | 2 |
| | | NDUFA11 | 2 | | | NEAT1 | 2 |
| | | NDUFA13 | 2 | | | SGK2 | 2 |
| | | LINC01485 | 2 | | | PDE4D | 2 |
| | | APOL1 | 2 | | | NNAT | 2 |
| | | MMAB | 2 | | | MAP2 | 2 |
| | | CLDN1 | 2 | | | ADCYAP1R1 | 2 |
| | | BCO2 | 2 | | | ZFP740 | 2 |
| | | MAOA | 2 | | | CLDN25 | 2 |
| | | MUT | 2 | | | PSAT1 | 2 |
| | | AKR1C2 | 2 | | | ARC | 2 |
| | | TMEM150A | 2 | | | GSS | 2 |
| | | RAB13 | 2 | | | TMOD1 | 2 |
| | | RAB17 | 2 | | | ZFP365 | 2 |
| | | GMPPA | 2 | | | NIPA1 | 2 |
| | | GAPDH | 2 | | | GOLGA7 | 2 |
| | | CMC2 | 2 | | | PTBP2 | 2 |
| | | KIF1C | 2 | | | SCAMP2 | 2 |
| | | ATP5F1 | 2 | | | FNTA | 2 |
| | | UQCR11 | 2 | | | EPB4.1L3 | 2 |
| | | TSKU | 2 | | | KIF26B | 2 |
| | | NDRG1 | 2 | | | TSPAN6 | 2 |
| | | SEC16B | 2 | | | TSPAN5 | 2 |
| | | GABARAPL1 | 2 | | | FDPS | 2 |
| | | TTPA | 2 | | | GLUL | 2 |
| | | ABHD14B | 2 | | | BCHE | 2 |
| | | ACMSD | 2 | | | GNG4 | 2 |
| | | ADH1B | 2 | | | OS9 | 2 |
| | | ADH1A | 2 | | | FAM102A | 2 |
| | | EPB41L4B | 2 | | | PLPPR1 | 2 |
| | | LURAP1L | 2 | | | C030030A07RIK | 2 |
| | | FURIN | 2 | | | ELMO1 | 2 |
| | | GADD45A | 2 | | | GPC6 | 2 |
| | | PARK7 | 2 | | | PPAPDC1A | 2 |
| | | SELENBP2 | 2 | | | REEP1 | 2 |
| | | ZYG11B | 2 | | | RGS16 | 2 |
| | | UGT2B7 | 2 | | | XKR4 | 2 |
| | | UGT2B4 | 2 | | | DICER1 | 2 |
| | | UGT2B5 | 2 | | | RAB31 | 2 |
| | | SLC25A20 | 2 | | | LRP1B | 2 |
| | | GOLT1A | 2 | | | ACAP3 | 2 |
| | | PC | 2 | | | MTAP | 2 |
| | | PEBP1 | 2 | | | BICD1 | 2 |
| | | GALK1 | 2 | | | PLEKHG1 | 2 |
| | | ADIPOR2 | 2 | | | | |
| | | LY6E | 2 | | | | |
| | | EFNA1 | 2 | | | | |
| | | OCLN | 2 | | | | |
| | | LRP6 | 2 | | | | |
| | | METTL7A | 2 | | | | |
| | | SEPP1 | 2 | | | | |
| | | PPP2R1B | 2 | | | | |
| | | ATP5G1 | 2 | | | | |
| | | SUCLG1 | 2 | | | | |
| | | PON3 | 2 | | | | |
| | | ABCC3 | 2 | | | | |
| | | MSMO1 | 2 | | | | |
| | | DHCR7 | 2 | | | | |
| | | GSN | 2 | | | | |
| | | CYP2F2 | 2 | | | | |
| | | MPST | 2 | | | | |
| | | KHK | 2 | | | | |
| | | CYP39A1 | 2 | | | | |
| | | NNT | 2 | | | | |
| | | MYO1B | 2 | | | | |
| | | PRAP1 | 2 | | | | |
| | | TFPI2 | 2 | | | | |
| | | CINP | 2 | | | | |
| | | SLC25A33 | 2 | | | | |
| | | MUP3 | 2 | | | | |
| | | TRAP1 | 2 | | | | |
| | | GYS2 | 2 | | | | |
| | | DENND5B | 2 | | | | |
| | | FMO3 | 2 | | | | |
| | | CYB5R3 | 2 | | | | |
| | | SORL1 | 2 | | | | |
| | | REEP6 | 2 | | | | |
| | | SLC13A5 | 2 | | | | |
| | | PBLD | 2 | | | | |
| | | GCAT | 2 | | | | |
| | | FAAH | 2 | | | | |
| | | LINC00261 | 2 | | | | |
| | | CD59 | 2 | | | | |
| | | DAO | 2 | | | | |
| | | DAP | 2 | | | | |

TABLE 10
List of sgRNA spacer sequences and
corresponding targets for mediating
CRISPR knockout, activation, and HDR.

| | | sgRNA target | |
|---|---|---|---|
| Target | | sequence | |
| gene | Name | (5′ to 3′) | Purpose |
| NEUROD1 | SAM sgRNA 1 | CATGCGCCAT | Activation |
| | | ATGGTCTTCC | |
| | | (SEQ ID | |
| | | NO: 10741) | |
| NEUROD1 | SAM sgRNA 2 | ATACAAATGG | Activation |
| | | GCAGGTCACG | |
| | | (SEQ ID | |
| | | NO: 10742) | |
| NEUROG2 | SAM sgRNA 1 | GAAAAGAATA | Activation |
| | | AGCCAGAGGA | |
| | | (SEQ ID | |
| | | NO: 10743) | |
| NEUROG2 | SAM sgRNA 2 | CTGACAGGAG | Activation |
| | | GAGGAGGCGG | |
| | | (SEQ ID | |
| | | NO: 10744) | |
| Non- | NT sgRNA 1 | CTGAAAAAGG | Activation; |
| targeting | | AAGGAGTTGA | knockout |
| | | (SEQ ID | |
| | | NO: 10745) | |
| Non- | NT sgRNA 2 | AAGATGAAAG | Activation; |
| targeting | | GAAAGGCGTT | knockout |
| | | (SEQ ID | |
| | | NO: 10746) | |
| SLC1A3 | sgRNA | ATGGAGAAGA | HDR |
| | | GCCCAAGA | |
| | | (SEQ ID | |
| | | NO: 10747) | |
| VIM | sgRNA | TCCGCAGCCA | HDR |
| | | TGTCCACC | |
| | | (SEQ ID | |
| | | NO: 10748) | |
| DYRK1A | KO sgRNA 1 | TCAGCAACCT | Knockout |
| | | CTAACTAACC | |
| | | (SEQ ID | |
| | | NO: 10749) | |
| DYRK1A | KO sgRNA 2 | TTACAGGAGT | Knockout |
| | | ACAAACCACC | |
| | | (SEQ ID | |
| | | NO: 10750) | |

TABLE 11
TaqMan qPCR probe ID's from Thermo Fisher
Scientific for detecting mRNA expression.

| Gene Name | RefSeq isoform(s) | Probe ID |
|---|---|---|
| NEUROD1 | NM_002500 | Hs01922995_s1 |
| NEUROG2 | NM_024019 | Hs00702774_s1 |
| SLC1A3 | NM_001166695; NM_001289939; NM_001289940; | Hs00904823_g1 |
| | NM_004172 | |
| VIM | NM_003380 | Hs00958111_m1 |
| TUBB3 | NM_001197181; NM_006086 | Hs00801390_s1 |
| MAP2 | NM_001039538; NM_002374; NM_031845; NM_031847 | Hs00258900_m1 |
| DYRK1A | NM_001396; NM_101395; NM_130436; NM_130438 | Hs00176369_m1 |

TABLE 12
Custom TaqMan qPCR probes for
detecting codon-optimized
DYRKIA ORF mRNA expression.

| Target | Primer | |
|---|---|---|
| Transcript | type | Sequence (5′ to 3′) |
| DYRK1A | Forward | ACACCTCAGGAGACCTGTAA |
| | | (SEQ ID NO: 10785) |
| DYRK1A | Probe | /56-FAM/AACTGACAG/ZEN/ |
| | | GAACTGCCGAAGTCC/3IABKFQ/ |
| | | (SEQ ID NO: 10786) |
| DYRK1A | Reverse | CCGAAGAGAAGCGCGATAAA |
| | | (SEQ ID NO: 10787) |

TABLE 13
Primers for PCR amplification used in this study.

| Name | Primer Sequence (5′ to 3′) | Purpose |
|---|---|---|
| SLC1A3 HDR Fwd 1 | TCCTGTAAAGCACCAGGAGG (SEQ ID NO: 10751) | SLC1A3 reporter line |
| | | genotyping |
| SLC1A3 HDR Fwd 2 | ACCAAAGAGGAGGTTTGGCT (SEQ ID NO: 10752) | SLC1A3 reporter line |
| | | genotyping |
| SLC1A3 HDR Rev | AGCAGCACAAAAGCATTCCG (SEQ ID NO: 10753) | SLC1A3 reporter line |
| | | genotyping |
| VIM HDR Fwd 1 | AGCCCGCTGAGACTTGAATC (SEQ ID NO: 10754) | VIM reporter line |
| | | genotyping |
| VIM HDR Fwd 2 | CTCGTTCGCCTCTTCTCCG (SEQ ID NO: 10755) | VIM reporter line |
| | | genotyping |
| VIM HDR Rev | GGTGGACGTAGTCACGTAGC (SEQ ID NO: 10756) | VIM reporter line |
| | | genotyping |
| TF NGS Fwd 1 | AATGATACGGCGACCACCGAGATCTACACTCTTTCCCTACACGA | TF library amplification for |
| | CGCTCTTCCGATCTTAAGTAGAGGCTTTATATATCTTGTGGAAA | NGS |
| | GGACGAAACACC (SEQ ID NO: 10757) | |
| TF NGS Fwd 2 | AATGATACGGCGACCACCGAGATCTACACTCTTTCCCTACACGA | TF library amplification for |
| | CGCTCTTCCGATCTATCATGCTTAGCTTTATATATCTTGTGGAAA | NGS |
| | GGACGAAACACC (SEQ ID NO: 10758) | |
| TF NGS Fwd 3 | AATGATACGGCGACCACCGAGATCTACACTCTTTCCCTACACGA | TF library amplification for |
| | CGCTCTTCCGATCTGATGCACATCTGCTTTATATATCTTGTGGAA | NGS |
| | AGGACGAAACACC (SEQ ID NO: 10759) | |
| TF NGS Fwd 4 | AATGATACGGCGACCACCGAGATCTACACTCTTTCCCTACACGA | TF library amplification for |
| | CGCTCTTCCGATCTCGATTGCTCGACGCTTTATATATCTTGTGGA | NGS |
| | AAGGACGAAACACC (SEQ ID NO: 10760) | |
| TF NGS Fwd 5 | AATGATACGGCGACCACCGAGATCTACACTCTTTCCCTACACGA | TF library amplification for |
| | CGCTCTTCCGATCTTCGATAGCAATTCGCTTTATATATCTTGTGG | NGS |
| | AAAGGACGAAACACC (SEQ ID NO: 10761) | |
| TF NGS Fwd 6 | AATGATACGGCGACCACCGAGATCTACACTCTTTCCCTACACGA | TF library amplification for |
| | CGCTCTTCCGATCTATCGATAGTTGCTTGCTTTATATATCTTGTG | NGS |
| | GAAAGGACGAAACACC (SEQ ID NO: 10762) | |
| TF NGS Fwd 7 | AATGATACGGCGACCACCGAGATCTACACTCTTTCCCTACACGA | TF library amplification for |
| | CGCTCTTCCGATCTGATCGATCCAGTTAGGCTTTATATATCTTGT | NGS |
| | GGAAAGGACGAAACACC (SEQ ID NO: 10763) | |
| TF NGS Fwd 8 | AATGATACGGCGACCACCGAGATCTACACTCTTTCCCTACACGA | TF library amplification for |
| | CGCTCTTCCGATCTCGATCGATTTGAGCCTGCTTTATATATCTTG | NGS |
| | TGGAAAGGACGAAACACC (SEQ ID NO: 10764) | |
| TF NGS Fwd 9 | AATGATACGGCGACCACCGAGATCTACACTCTTTCCCTACACGA | TF library amplification for |
| | CGCTCTTCCGATCTACGATCGATACACGATCGCTTTATATATCTT | NGS |
| | GTGGAAAGGACGAAACACC (SEQ ID NO: 10765) | |
| TF NGS Fwd 10 | AATGATACGGCGACCACCGAGATCTACACTCTTTCCCTACACGA | TF library amplification for |
| | CGCTCTTCCGATCTTACGATCGATGGTCCAGAGCTTTATATATCT | NGS |
| | TGTGGAAAGGACGAAACACC (SEQ ID NO: 10766) | |
| TF NGS Rev 1 | CAAGCAGAAGACGGCATACGAGATTCGCCTTGGTGACTGGAGT | TF library amplification for |
| | TCAGACGTGTGCTCTTCCGATCTTAAAGCAGCGTATCCACATAG | NGS |
| | CGT (SEQ ID NO: 10767) | |
| TF NGS Rev 2 | CAAGCAGAAGACGGCATACGAGATATAGCGTCGTGACTGGAG | TF library amplification for |
| | TTCAGACGTGTGCTCTTCCGATCTTAAAGCAGCGTATCCACATA | NGS |
| | GCGT (SEQ ID NO: 10768) | |
| TF NGS Rev 3 | CAAGCAGAAGACGGCATACGAGATGAAGAAGTGTGACTGGAG | TF library amplification for |
| | TTCAGACGTGTGCTCTTCCGATCTTAAAGCAGCGTATCCACATA | NGS |
| | GCGT (SEQ ID NO: 10769) | |
| TF NGS Rev 4 | CAAGCAGAAGACGGCATACGAGATATTCTAGGGTGACTGGAGT | TF library amplification for |
| | TCAGACGTGTGCTCTTCCGATCTTAAAGCAGCGTATCCACATAG | NGS |
| | CGT (SEQ ID NO: 10770) | |
| TF NGS Rev 5 | CAAGCAGAAGACGGCATACGAGATCGTTACCAGTGACTGGAGT | TF library amplification for |
| | TCAGACGTGTGCTCTTCCGATCTTAAAGCAGCGTATCCACATAG | NGS |
| | CGT (SEQ ID NO: 10771) | |
| TF NGS Rev 6 | CAAGCAGAAGACGGCATACGAGATGTCTGATGGTGACTGGAG | TF library amplification for |
| | TTCAGACGTGTGCTCTTCCGATCTTAAAGCAGCGTATCCACATA | NGS |
| | GCGT (SEQ ID NO: 10772) | |
| TF NGS Rev 7 | CAAGCAGAAGACGGCATACGAGATTTACGCACGTGACTGGAGT | TF library amplification for |
| | TCAGACGTGTGCTCTTCCGATCTTAAAGCAGCGTATCCACATAG | NGS |
| | CGT (SEQ ID NO: 10773) | |
| TF NGS Rev 8 | CAAGCAGAAGACGGCATACGAGATTTGAATAGGTGACTGGAG | TF library amplification for |
| | TTCAGACGTGTGCTCTTCCGATCTTAAAGCAGCGTATCCACATA | NGS |
| | GCGT (SEQ ID NO: 10774) | |
| TF NGS Rev 9 | CAAGCAGAAGACGGCATACGAGATTCCTTGGTGTGACTGGAGT | TF library amplification for |
| | TCAGACGTGTGCTCTTCCGATCTTAAAGCAGCGTATCCACATAG | NGS |
| | CGT (SEQ ID NO: 10775) | |
| TF NGS Rev 10 | CAAGCAGAAGACGGCATACGAGATACAGGTATGTGACTGGAG | TF library amplification for |
| | TTCAGACGTGTGCTCTTCCGATCTTAAAGCAGCGTATCCACATA | NGS |
| | GCGT (SEQ ID NO: 10776) | |
| TF NGS Rev 11 | CAAGCAGAAGACGGCATACGAGATAGGTAAGGGTGACTGGAG | TF library amplification for |
| | TTCAGACGTGTGCTCTTCCGATCTTAAAGCAGCGTATCCACATA | NGS |
| | GCGT (SEQ ID NO: 10777) | |
| TF NGS Rev 12 | CAAGCAGAAGACGGCATACGAGATAACAATGGGTGACTGGAG | TF library amplification for |
| | TTCAGACGTGTGCTCTTCCGATCTTAAAGCAGCGTATCCACATA | NGS |
| | GCGT (SEQ ID NO: 10778) | |
| TF Perturb Fwd | AATGATACGGCGACCACCGAGATCTACACTCTTTCCCTACACGA | Perturb-seq TF and cell |
| | CGCTCTTCCGATCT (SEQ ID NO: 10779) | barcode amplification for |
| | | NGS |
| TF Perturb Rev | TTGGCTTTATATATCTTGTGGAAAGGACGA | Perturb-seq TF and cell |
| | (SEQ ID NO: 10780) | barcode amplification for |
| | | NGS |
| DYRK1A KO | CTTTCCCTACACGACGCTCTTCCGATCTCGTCGCCAGCCAAACAT | DYRK1A knockout indel |
| sgRNA 1 | AAG (SEQ ID NO: 10781) | analysis |
| Fwd | | |
| DYRK1A KO | GACTGGAGTTCAGACGTGTGCTCTTCCGATCTCCCACGTTGCAT | DYRK1A knockout indel |
| sgRNA 1 | GTAAAACTGAA (SEQ ID NO: 10782) | analysis |
| Rev | | |
| DYRK1A KO | CTTTCCCTACACGACGCTCTTCCGATCTATCGGTCTCCAGAGGT | DYRK1A knockout indel |
| sgRNA 2 | GCTA (SEQ ID NO: 10783) | analysis |
| Fwd | | |
| DYRK1A KO | GACTGGAGTTCAGACGTGTGCTCTTCCGATCTAGACATCTTTGC | DYRK1A knockout indel |
| sgRNA 2 | CTAAAAACAACTT (SEQ ID NO: 10784) | analysis |
| Rev | | |

TABLE 14
FISH probes.

| Gene | | |
|---|---|---|
| Name | RefSeq isoform(s) | Probe ID |
| RPL13A | NM_001270491; NM_012423 | VA10-19016-PF |
| SLC1A3 | NM_001166695; NM_001166696; NM_001289939; NM_001289940; | VA4-3088280- |
| | NM_004172 | PF |
| VIM | NM_003380 | VA4-14372-PF |
| PTN | NM_001321386; NM_001321387; NM_002825 | VA4-3087919- |
| | | PF |
| NCAN | NM_004386 | VA4-3086032- |
| | | PF |
| FABP7 | NM_001319039; NM_001319041; NM_001319042; NM_001446 | VA4-3086347- |
| | | PF |
| CLU | NM_001831 | VA1-11566-PF |
| CKB | NM_001823 | VA1-3000320- |
| | | PF |
| NR2E1 | NM_001286102; NM_003269 | VA1-3004772- |
| | | PF |
| TTYH1 | NM_001005367; NM_001201461; NM_020659 | VA1-3000024- |
| | | PF |
| F3 | NM_001178096; NM_001993 | VA1-19910-PF |

### Example 22—Transcription Factor Atlas of Directed Differentiation

Achieving a comprehensive understanding of the gene regulatory networks that govern cell states is a fundamental goal in molecular cell biology. Transcription factors (TFs) can bind to specific sequences in the genome to regulate the expression of ensembles of genes. Some TFs function as “master regulators” that exert control over processes that specify cell types and alter cell states (1-5). Perturbing TFs, especially by overexpression, can thus offer a simpler way to guide cell states than perturbing their downstream genes.

Generation of diverse cell types has the potential to realize a broad array of cellular replacement therapies and provide tractable models that can be perturbed, genetically or chemically, to assess effects in a cell type-specific context (6-20). However, it remains challenging or impossible to generate many cell types. The best differentiation methods are often labor-intensive and can require months to produce even heterogenous or immature cell populations. Overexpression of TFs can direct differentiation of pluripotent stem cells towards many different cell types (21-28), including neurons (24, 28) and skeletal muscle cells (26), or reprogram differentiated cells, such as fibroblasts, into other cell types, such as stem cells (25, 27) or neurons (22). Compared to exogenous growth factors or small molecules, which go through a TF intermediary to affect gene expression, directly overexpressing TFs to drive differentiation may enhance efficiency and reduce variability (29). As TF overexpression often mimics natural developmental processes, this approach should in theory be capable of generating all possible cell types. Elucidating the gene programs regulated by TFs will guide the production of diverse cellular models with higher fidelity while illuminating aspects of development. Here, Applicants sought to systematically map expression changes driven by TFs and identify TFs and their combinations that govern specific cellular differentiation programs in human embryonic stem cells (hESCs) by developing a TF overexpression screening platform for high-throughput interrogation of TF function.

Transcription factors (TFs) regulate gene programs, thereby controlling diverse cellular processes and cell states. To achieve a comprehensive understanding of TFs and their respective programs, Applicants developed a platform for high-throughput, systematic TF ORF overexpression that leverages barcodes for pooled screening. Applicants created a library of all annotated human TF splice isoforms (1,836 genes encoding 3,548 isoforms) and applied it to build a TF Atlas charting expression profiles in human embryonic stem cells (hESCs) overexpressing each TF. The comprehensive TF Atlas allowed systematic investigation and generalized observations, showing that 27% of TF genes could function as “master regulators” that induce differentiation when overexpressed in hESCs. Applicants mapped TF-induced expression profiles to reference cell types and validated candidate TFs for generation of diverse cell types, spanning all three germ layers and trophoblasts. Further targeted screens with a subset of the library allowed us to create a tailored cellular disease model and integrate mRNA expression and chromatin accessibility data to identify downstream regulators. Finally, Applicants predicted the effects of TF combinations, demonstrated the validity of Applicants' predictions in a combinatorial TF overexpression dataset, and showed how to predict combinations of TFs that could produce target profiles of reference cell types, reducing the combinatorial search space for experiments. The TF atlas provides a comprehensive overview of gene regulatory networks and a roadmap for further understanding developmental trajectories and guiding cellular engineering efforts.

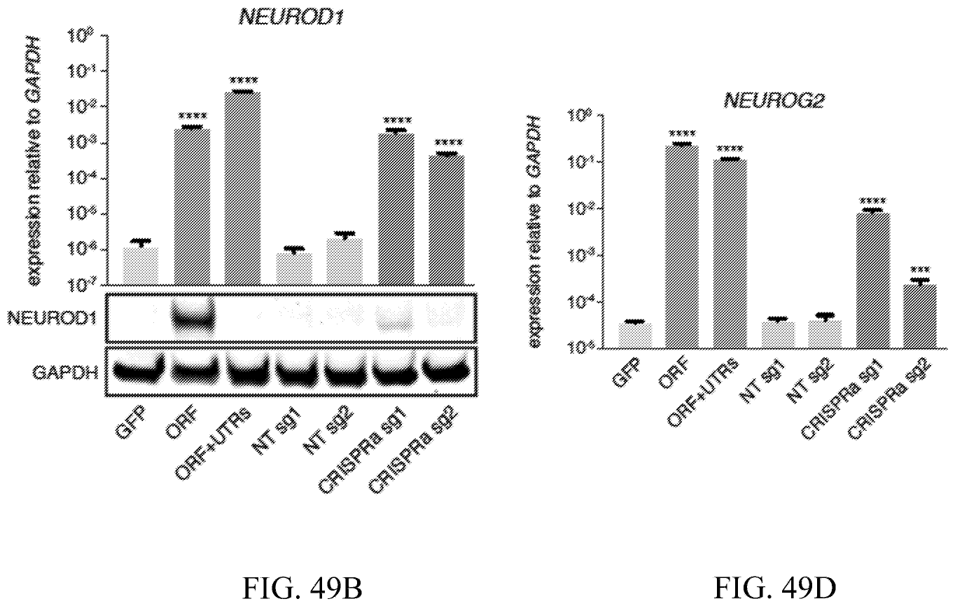

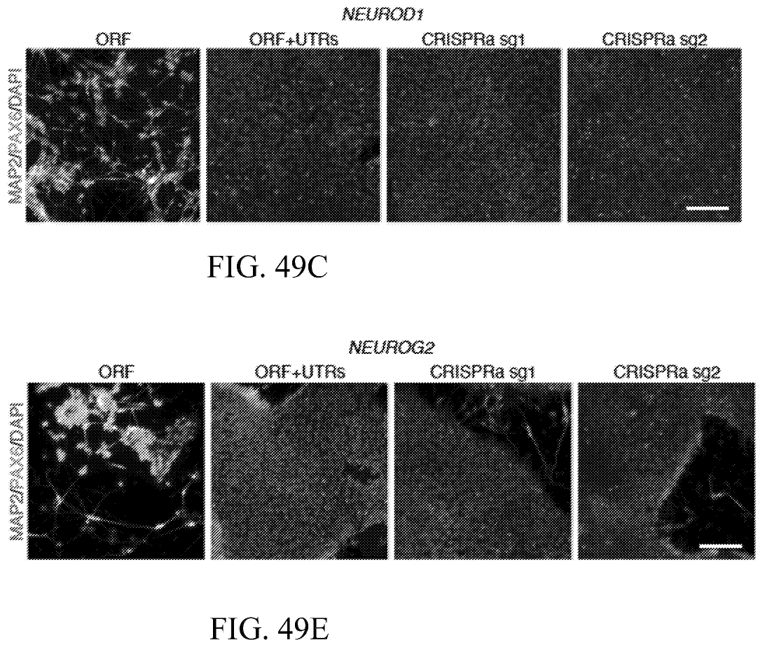

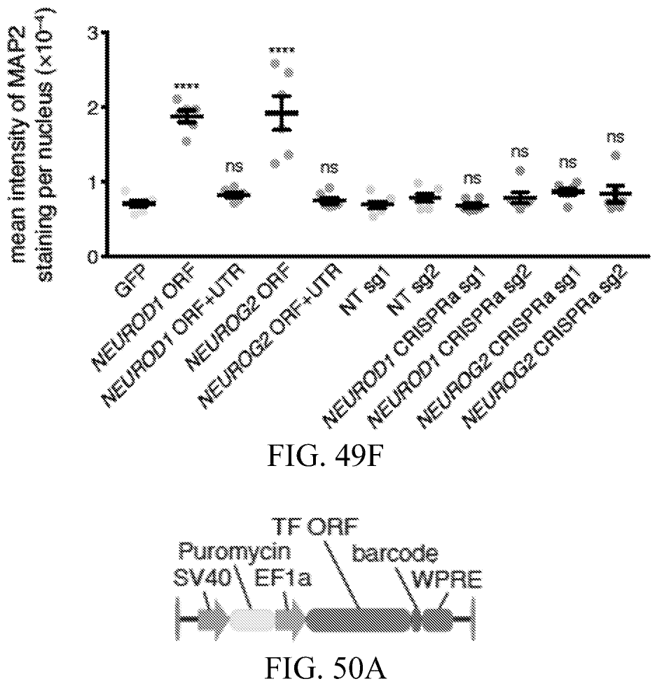

Development of Multiplexed Overexpression of Regulatory Factors (MORF) library. Applicants first established the most efficient mode of TF upregulation by comparing the ability of CRISPR activation (CRISPRa) (30) and ORF-based methods using overexpression of NEUROD1 or NEUROG2 to induce neuronal differentiation in HUES66 hESCs as a test case (FIG. 49A) (28). ORF expression of both TFs effectively induced neuronal differentiation, but TF upregulation using CRISPRa or ORFs with endogenous UTRs did not, despite robust upregulation of expression (FIG. 49B-F). This may indicate that hESCs have post-transcriptional regulatory mechanisms in endogenous UTRs that buffer against TF protein expression. Applicants therefore proceeded with TF ORF overexpression for screening.

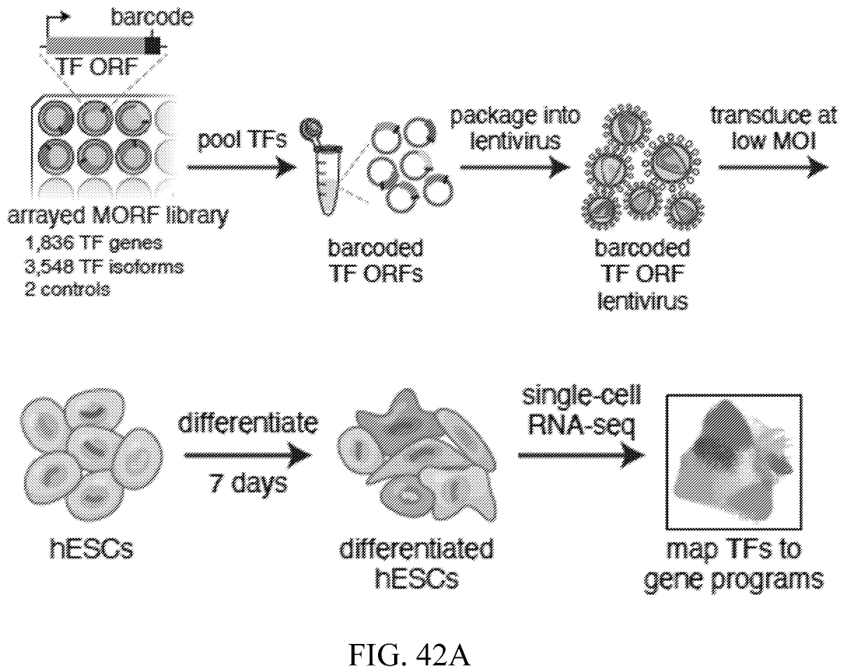

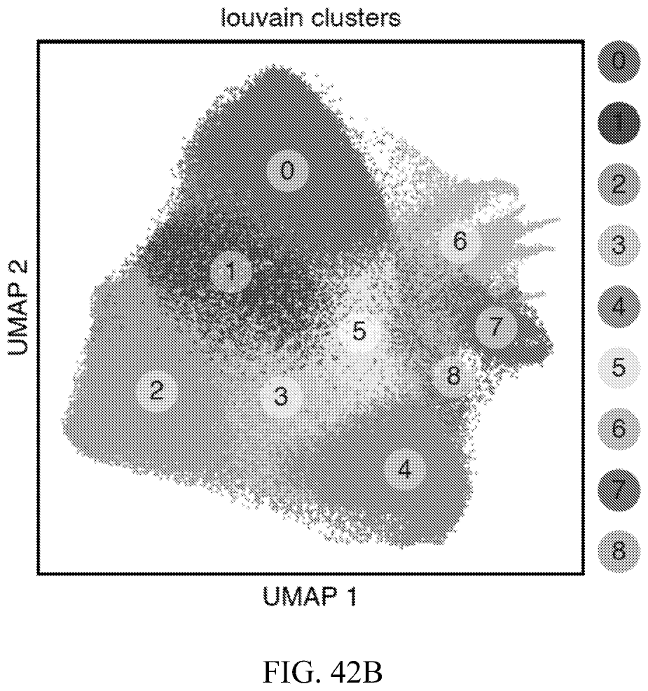

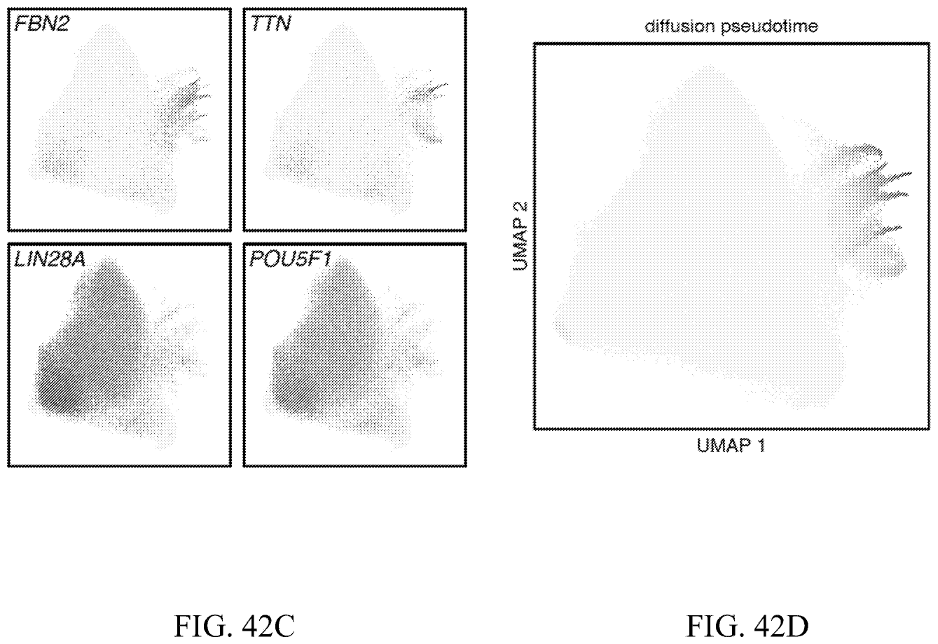

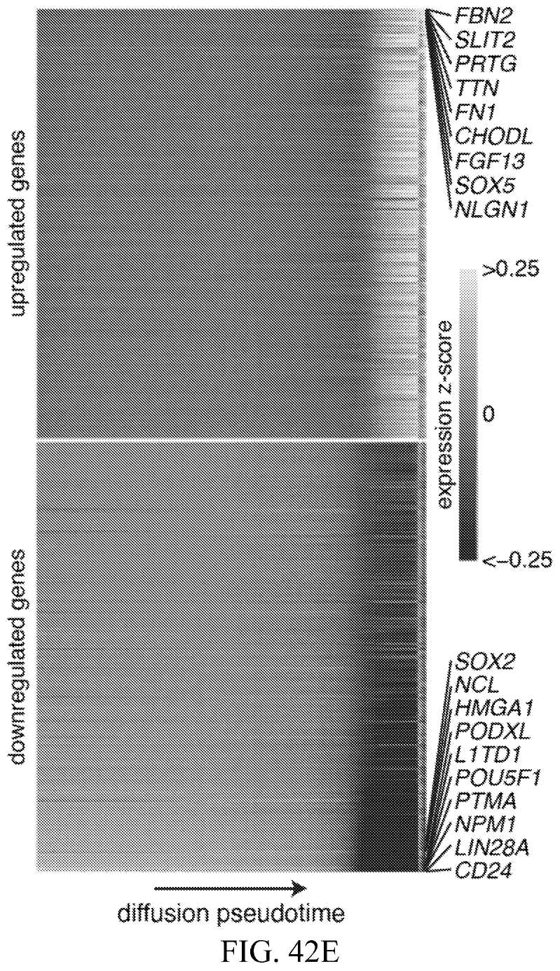

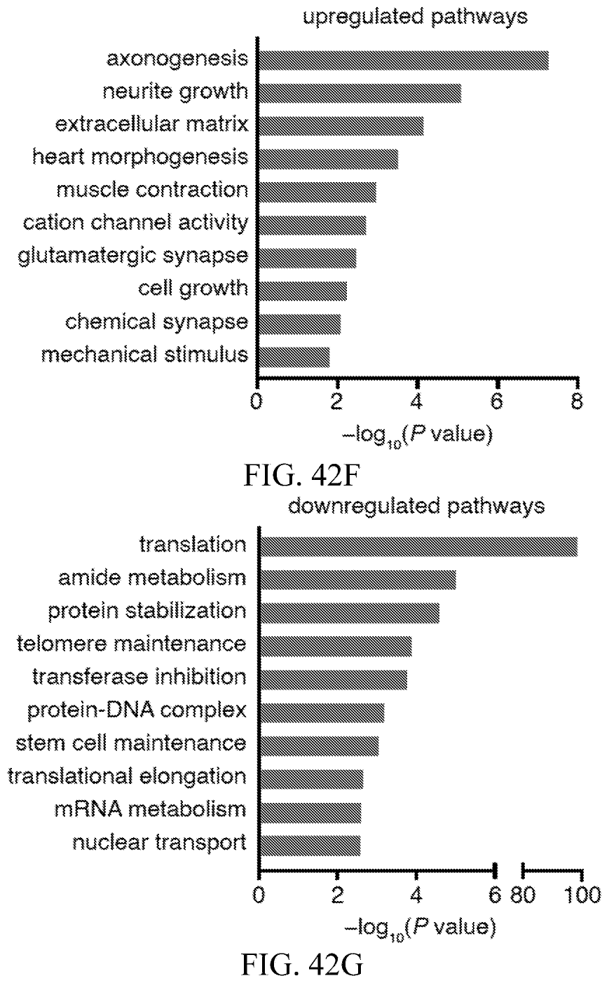

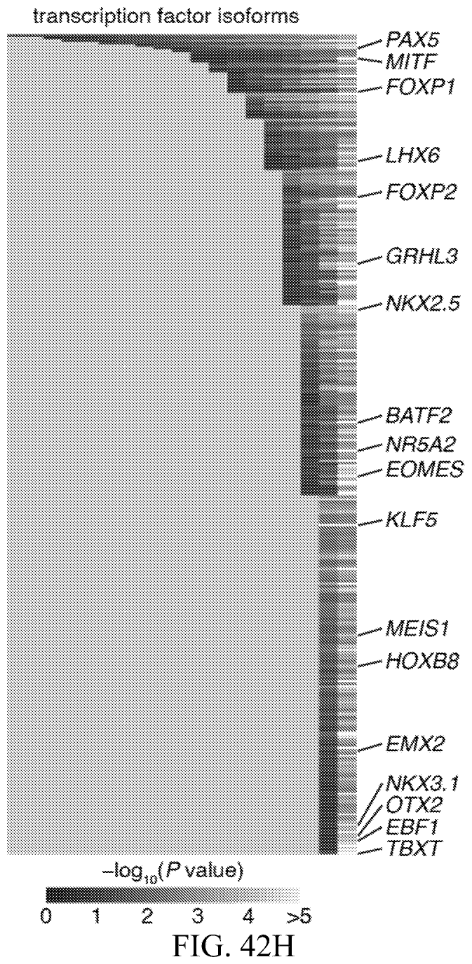

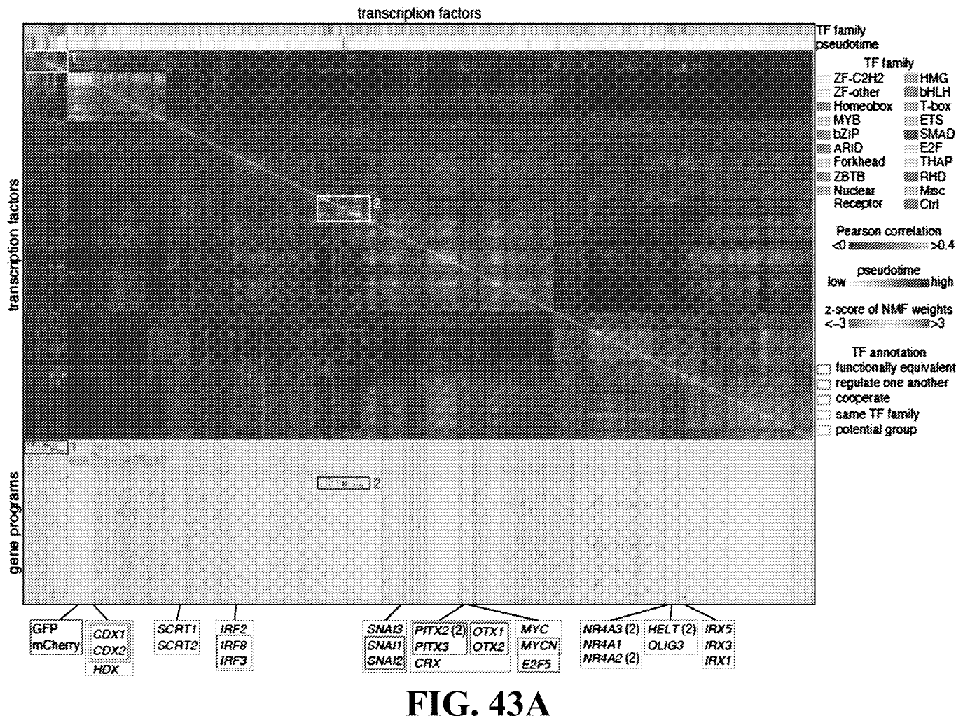

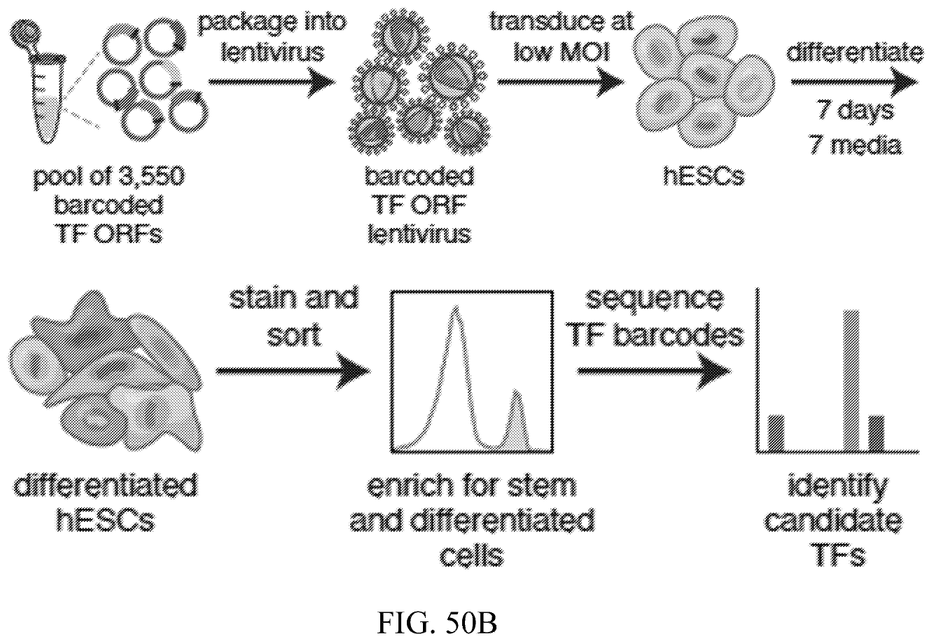

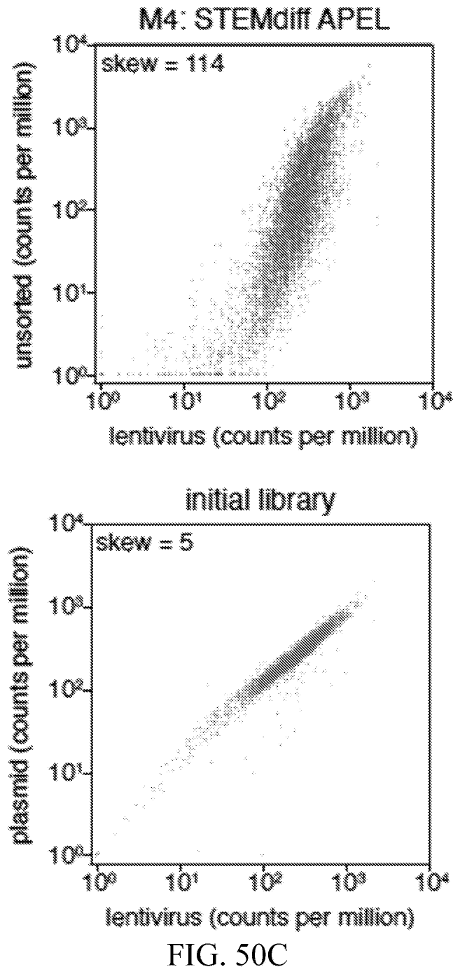

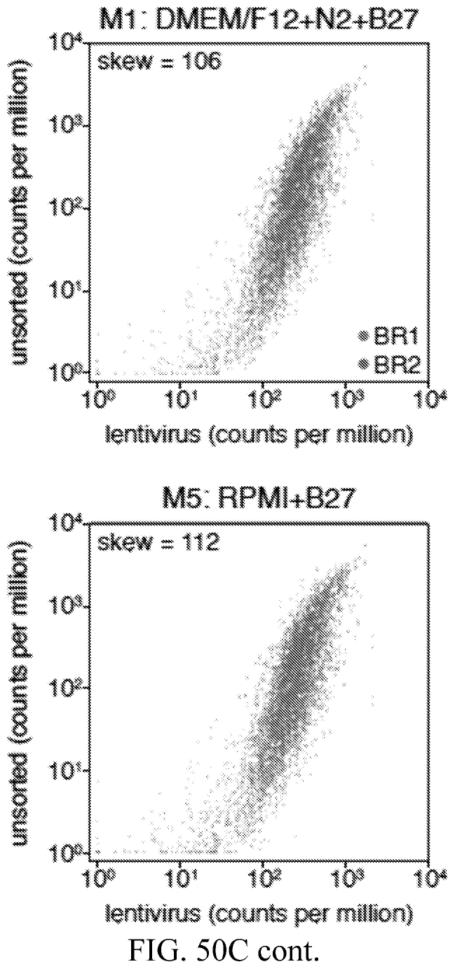

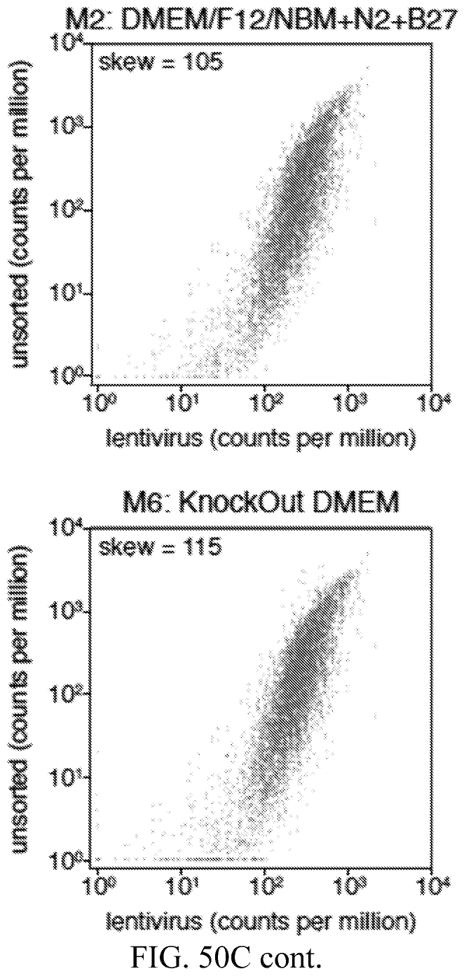

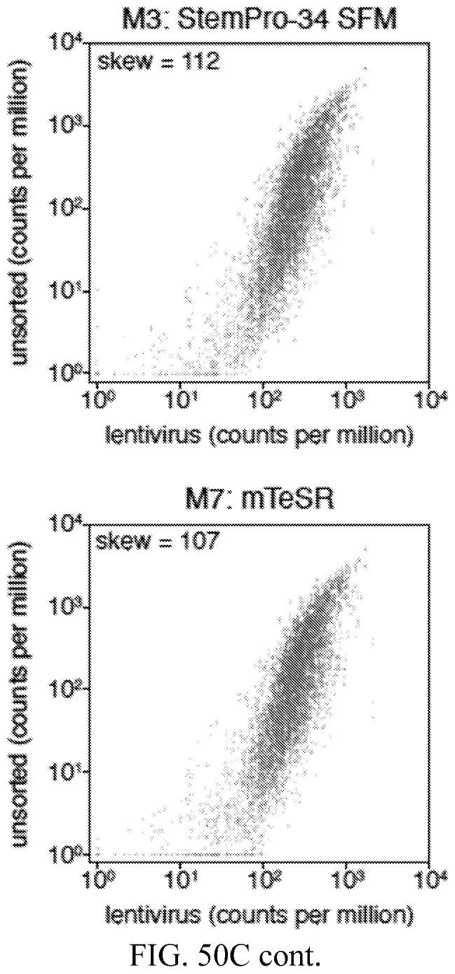

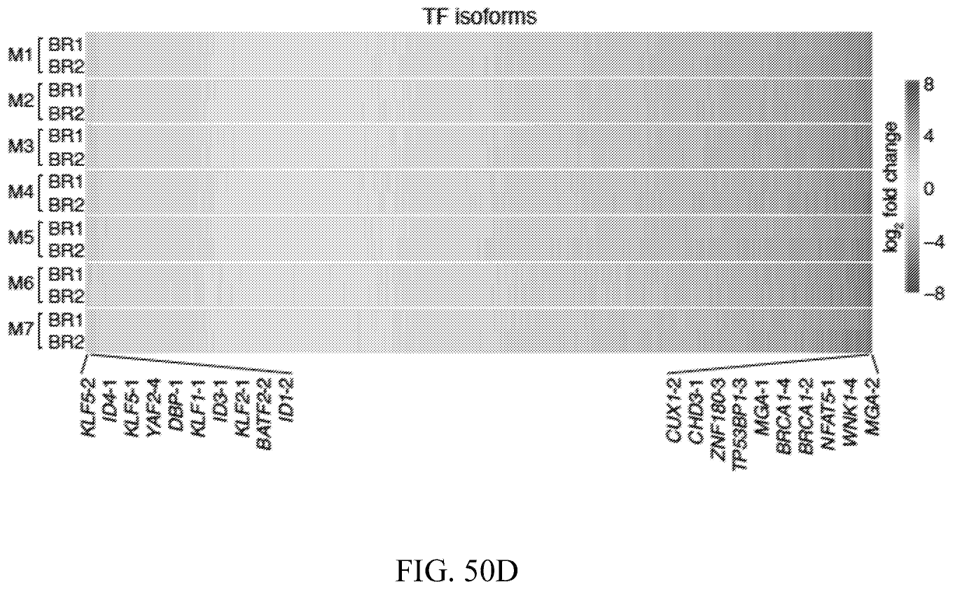

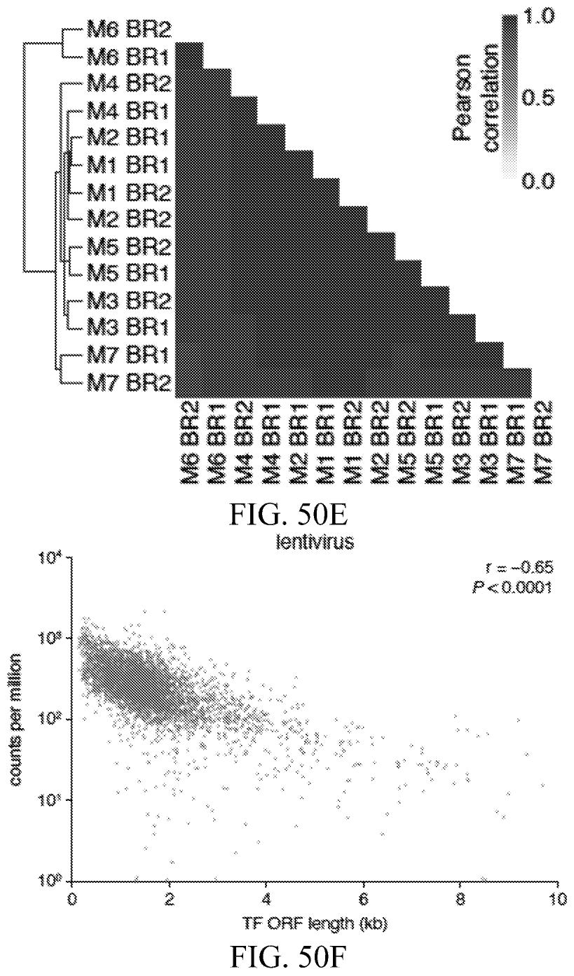

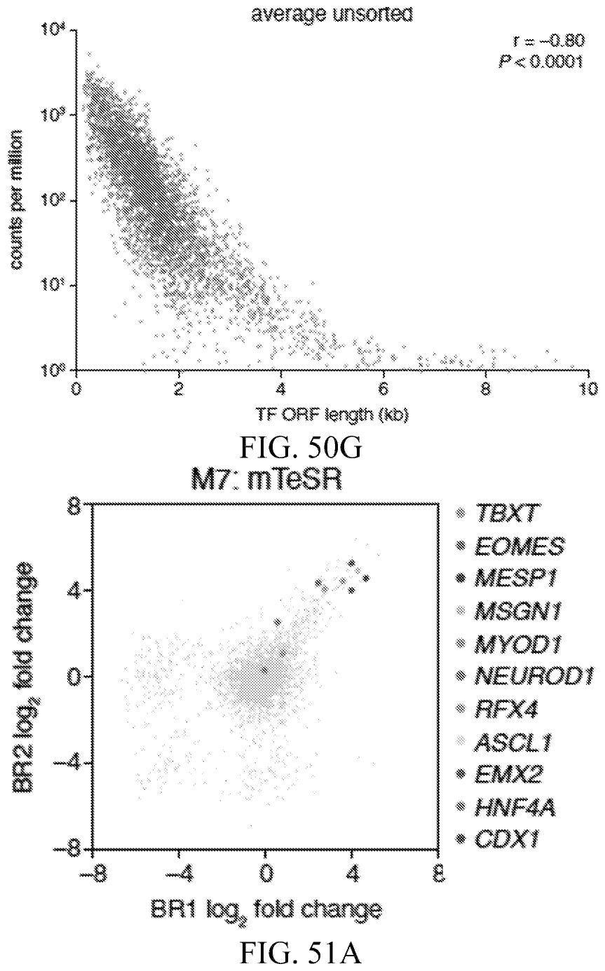

To enable pooled screening, Applicants created a barcoded human TF library, which Applicants named Multiplexed Overexpression of Regulatory Factors (MORF) (FIG. 42A, FIG. 50A, and Table 3). The MORF library consists of 3,548 splice isoforms encoded by 1,836 genes, including histone modifiers, considering all overlapping RefSeq and Gencode annotations, as choice of isoform has been shown to affect differentiation efficiency (21). As ORF libraries generated from cDNA libraries often contain missense mutations that can result in screening artifacts, Applicants individually synthesized and sequence verified all constructs in the MORF library.

MORF has several advantages over prior libraries. First, it is the most comprehensive TF library to date, compared to 1,732 isoforms in a recent collection (21). Furthermore, in contrast to the previous collection, the MORF library vectors contain unique barcodes that facilitate isoform identification and minimize ORF length-dependent PCR bias that could confound screening results (31). Applicants' MORF library design minimized barcode shuffling rates observed in another previous design (23), by reducing the distance between barcode and TF (FIG. 50A). Finally, as an arrayed library of all annotated human TFs that could be selectively pooled for targeted screening, MORF is a generalizable resource that enables the comprehensive discovery of TFs that induce phenotypes of interest.

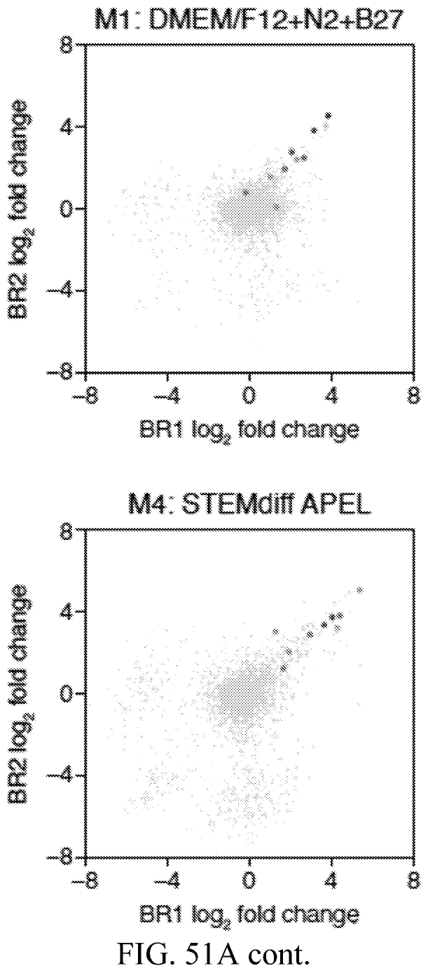

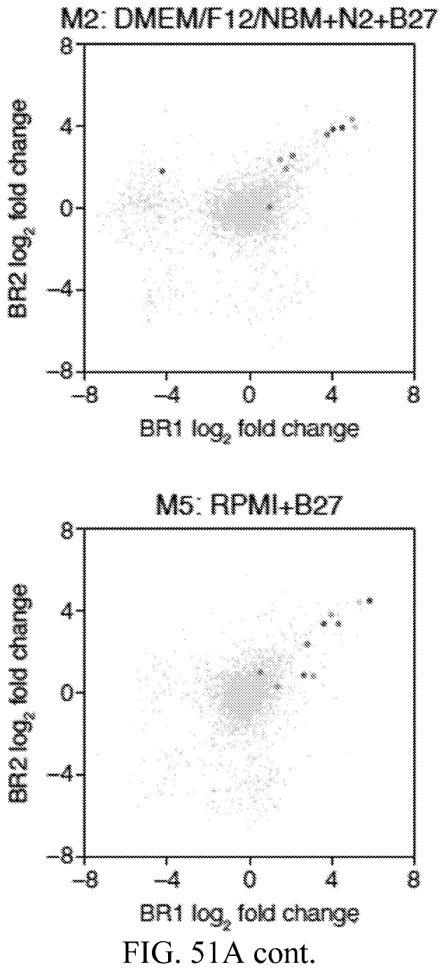

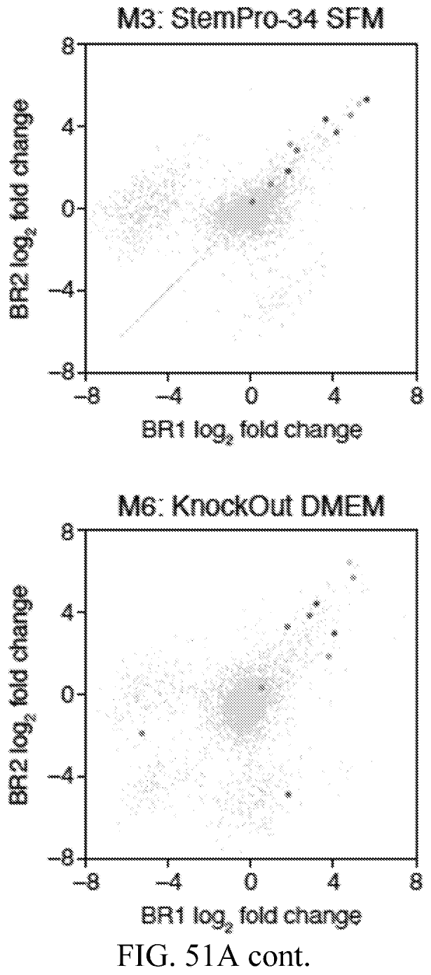

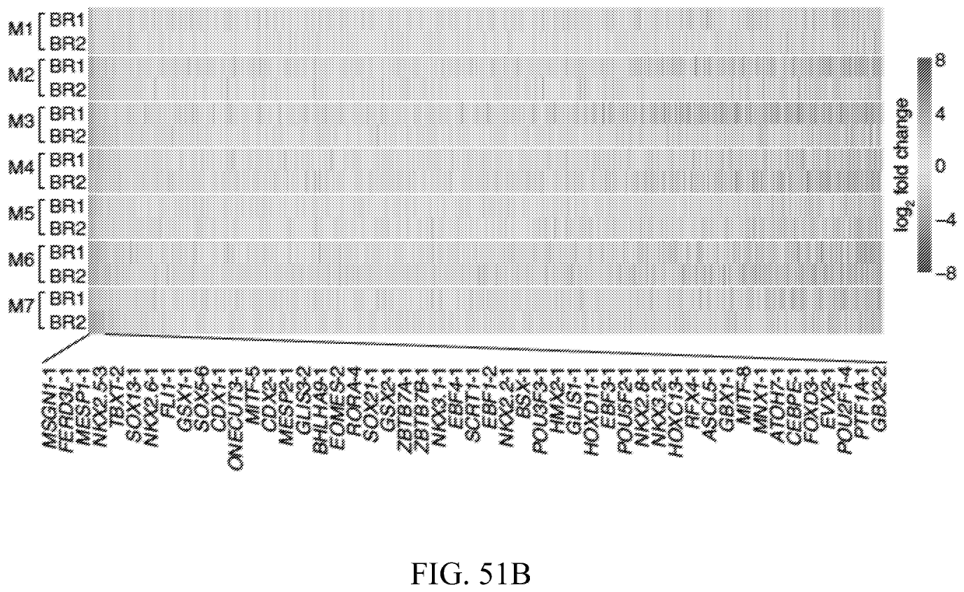

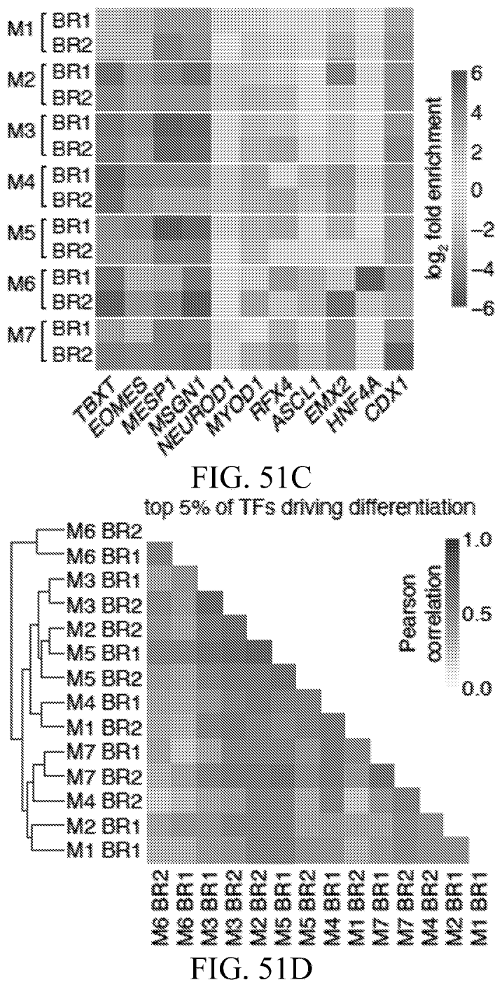

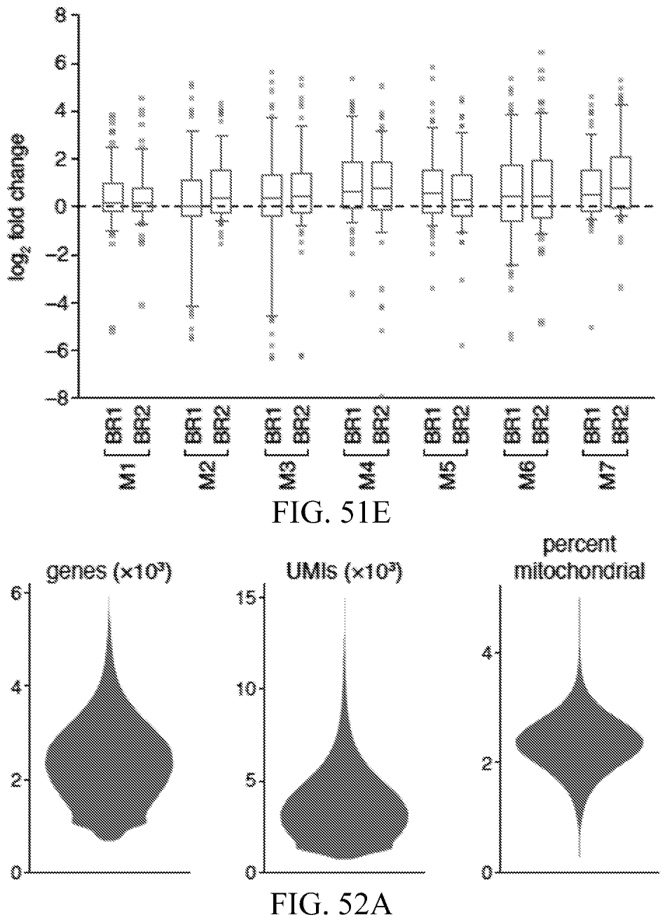

Construction of a TF atlas of directed differentiation. Applicants first applied MORF to comprehensively test which and how many TFs can drive cell fate changes in directed differentiation. Because existing protocols for cellular differentiation often use different culture media, depending on the target cell type (28, 32-34), Applicants first identified an optimal cell culture media that could capture the broadest range of effects of TFs. To this end, Applicants pooled the MORF library TFs and packaged them into a lentiviral library for delivery in H1 hESCs. Applicants tested 7 media conditions selected from published reprogramming protocols for cell types of different lineages (see Methods; FIG. 50B). After 7 days, for each media condition, Applicants sorted cells into two populations (top and bottom 10%) based on the expression level of pluripotency markers (TRA-1-60 and SSEA4), as a proxy for differentiation, and sequenced the TF barcodes in the unsorted and sorted bulk populations. Despite initial even distributions in the plasmid and lentiviral libraries (skew=5), in the unsorted populations, the TF distributions became very skewed across all media conditions (skew=105-115; FIG. 50C). As the TF distributions were remarkably consistent across replicates and media conditions (Pearson r >0.94; FIG. 50D, E), Applicants reasoned that the increase in skew is likely a result of TF-dependent effects on cell fitness. TFs that promote pluripotency maintenance (e.g., KLFs 1, 2, and 5; IDs 1, 3, and 4; and YAF2) (35-37) increased cell fitness (i.e., the TFs were overrepresented in the unsorted populations), whereas TFs involved in DNA damage sensing and repair (e.g., BRCA1 and TP53BP1) (38), decreased cell fitness (FIG. 50D). TF distributions in the lentivirus library and unsorted populations negatively correlated with TF length, suggesting that the packaging and integration efficiency may be lower for larger genes (FIG. 50F, G). Similar to the unsorted populations, TF distributions in the sorted populations were relatively consistent across media conditions (FIG. 51A-D). Out of the top 5% of TFs driving differentiation, 94% were reproducible across 3 or more media conditions (average Pearson r=0.51), suggesting that cell culture media does not strongly influence differentiation outcome. TFs enriched in the differentiated cell population (i.e., the bottom 10% relative to the top 10%) included developmentally critical TFs, such as MSGN1, TBXT, and CDX1 (FIG. 51B). By examining the enrichment of known developmentally critical TFs (23), Applicants selected the media condition that produced the highest enrichment and most even distribution, STEMdiff APEL (FIG. 51C, E).

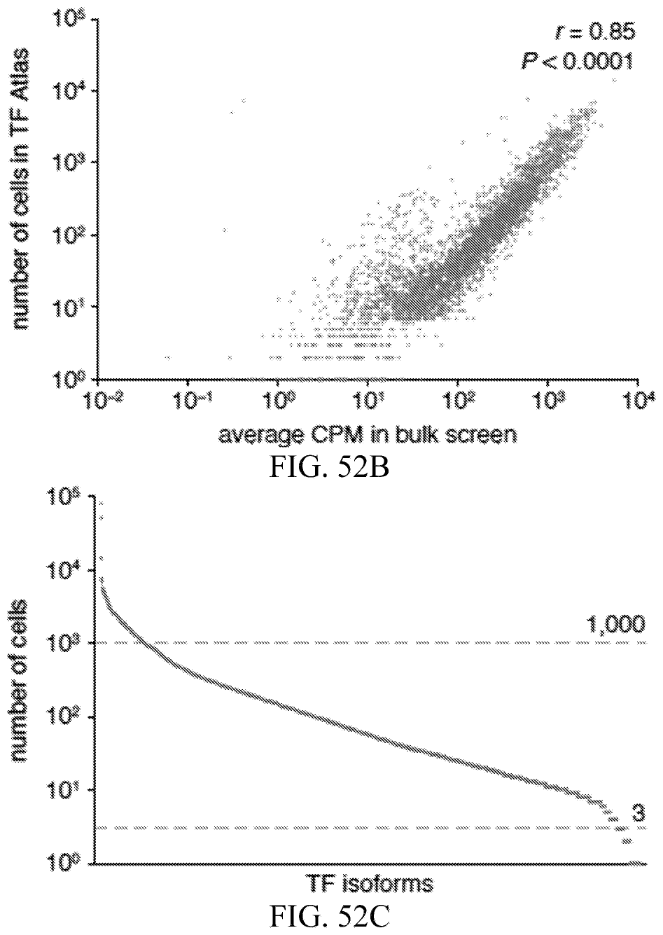

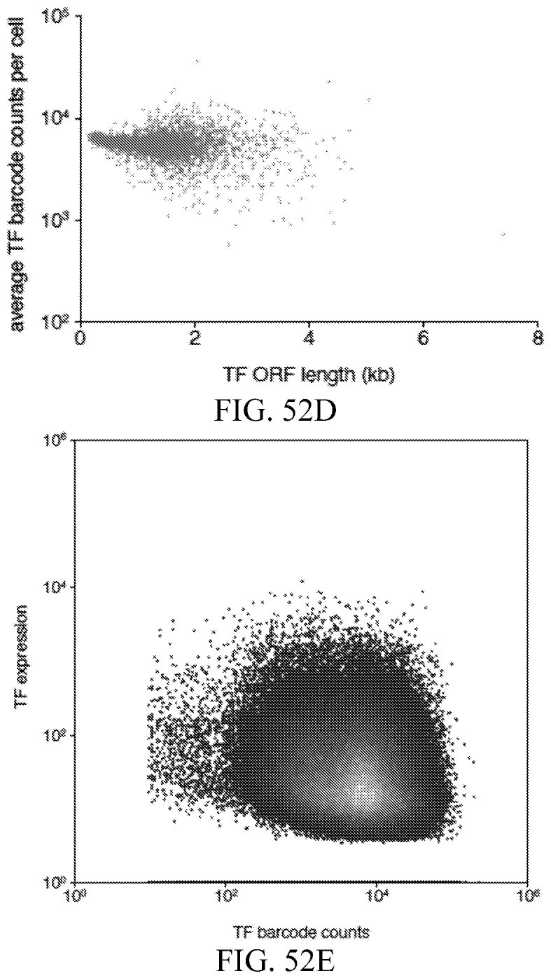

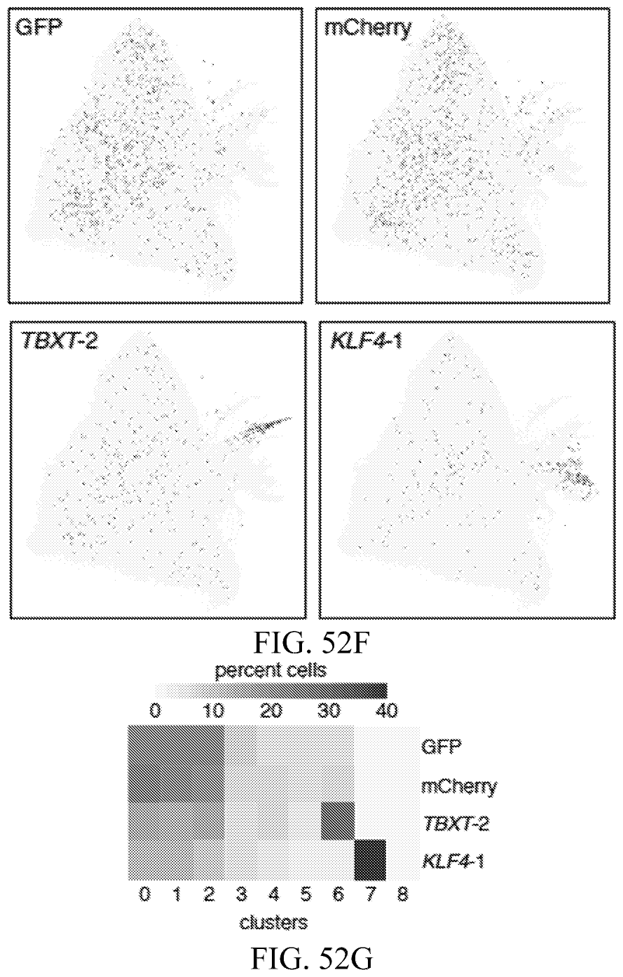

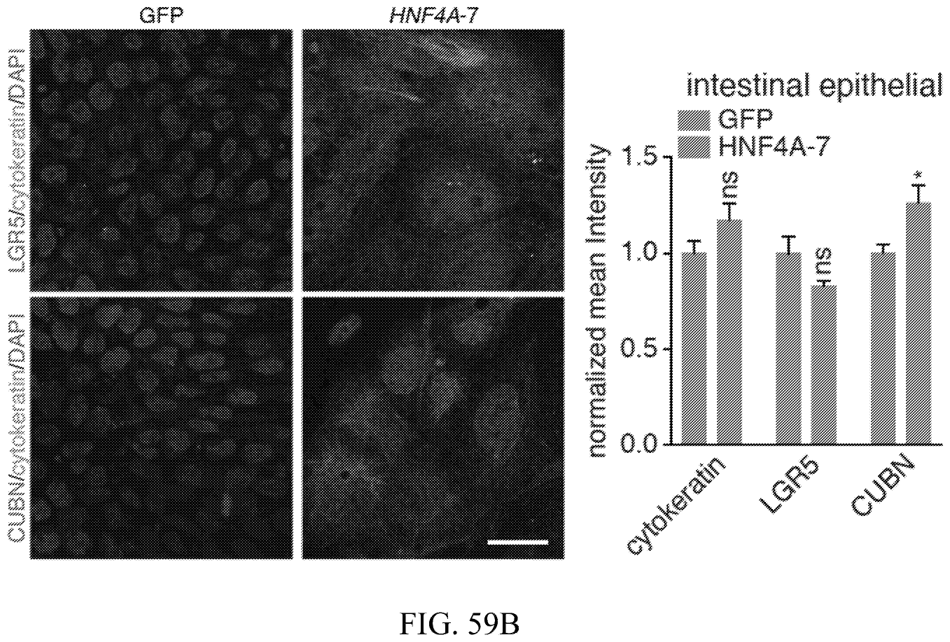

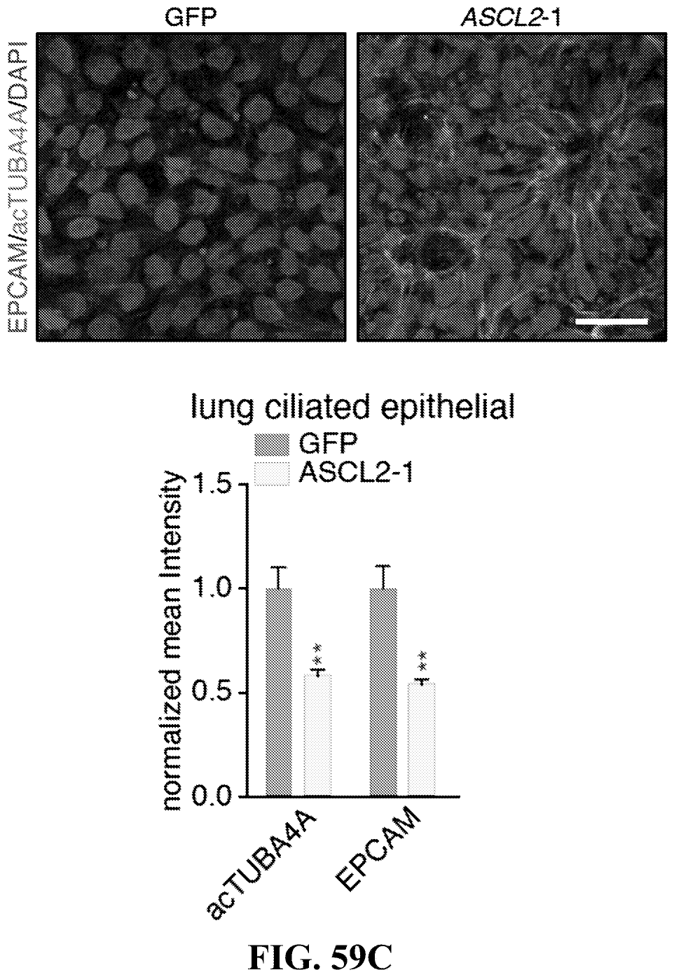

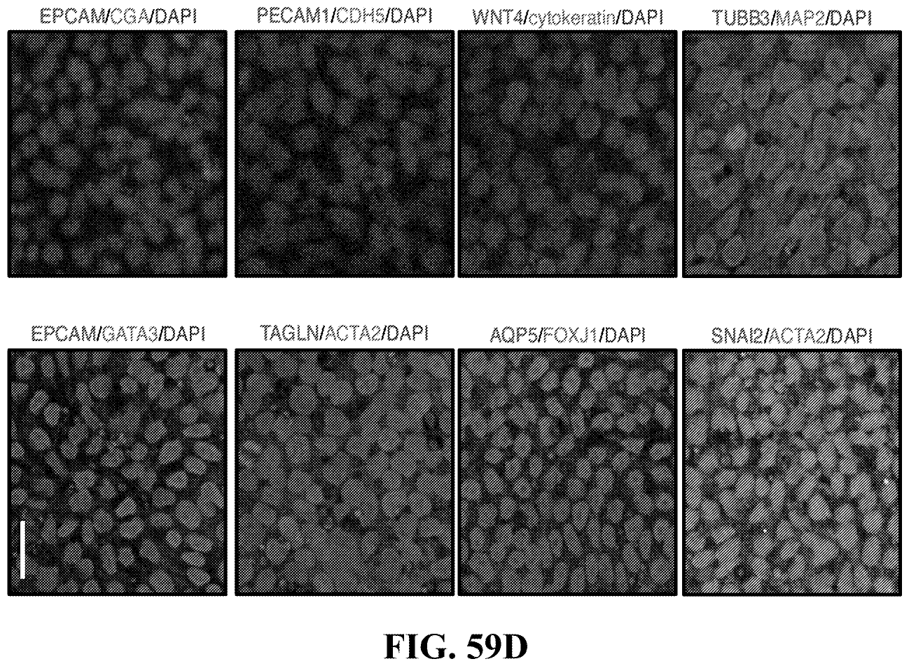

To build an expression atlas of all TF overexpression effects, Applicants transduced hESCs with the MORF library, differentiated cells in STEMdiff APEL media for 7 days, and profiled the cells by single cell RNA-Seq (scRNA-seq), using a combinatorial indexing protocol based on SHARE-seq (FIG. 42A; see Methods) (39). After filtering for quality and TF barcode mapping, Applicants retained >1.1 million high quality cell profiles (3,761 UMIs per cell on average) with a comparable TF distribution to the bulk TF screen, and no strong dependence between TF detectability and number of UMIs per cell (FIG. 52A-C). Applicants then down-sampled the data by TF ORF to 671,453 cells covering 3,266 TFs (92% of the MORF library; FIG. 42B and FIG. 52C; see Methods) to ensure even representation (3-1,000 cells, with an average of 206 cells, per TF ORF). Expression level of TF ORFs did not correlate with TF length or expression of the respective endogenous TF, as the TF ORF sequence is too distant (>1 kb) from the 3′ end of the transcript to be captured by SHARE-seq (FIG. 50A and FIG. 59D, E). This allows us to decouple expression of the TF ORF from the corresponding endogenous TF and observe potential positive feedback mechanisms, as many TFs are known to regulate themselves (40).

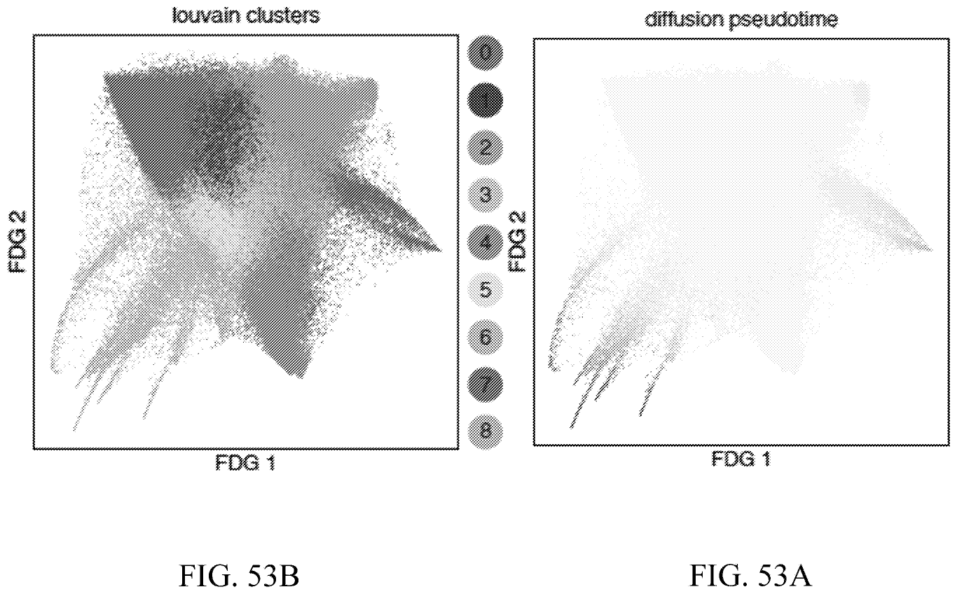

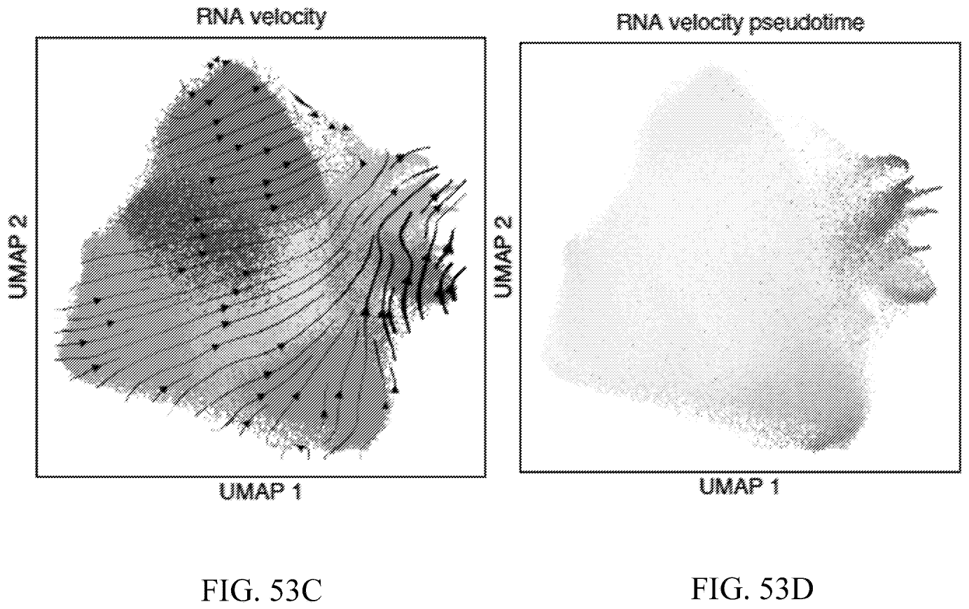

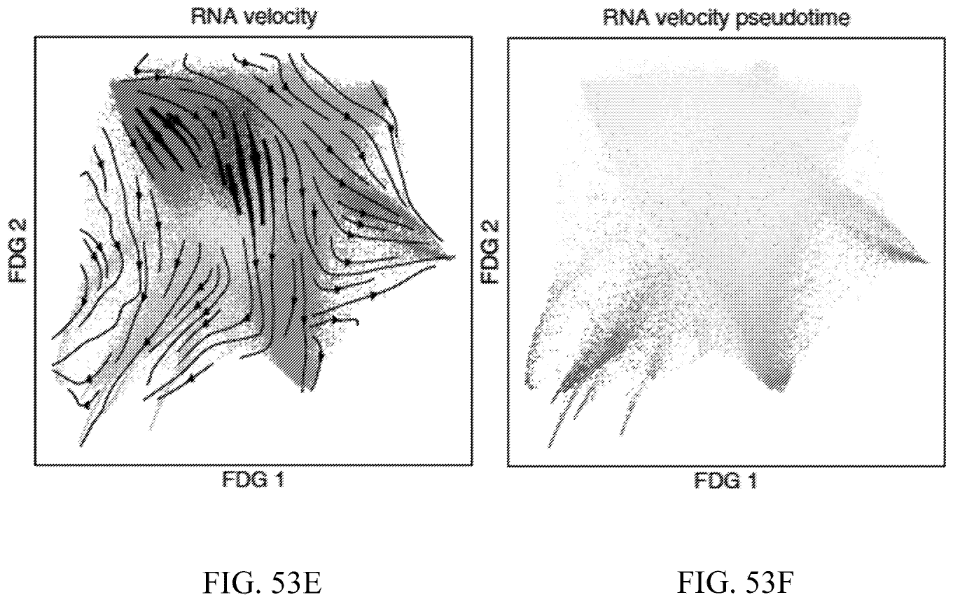

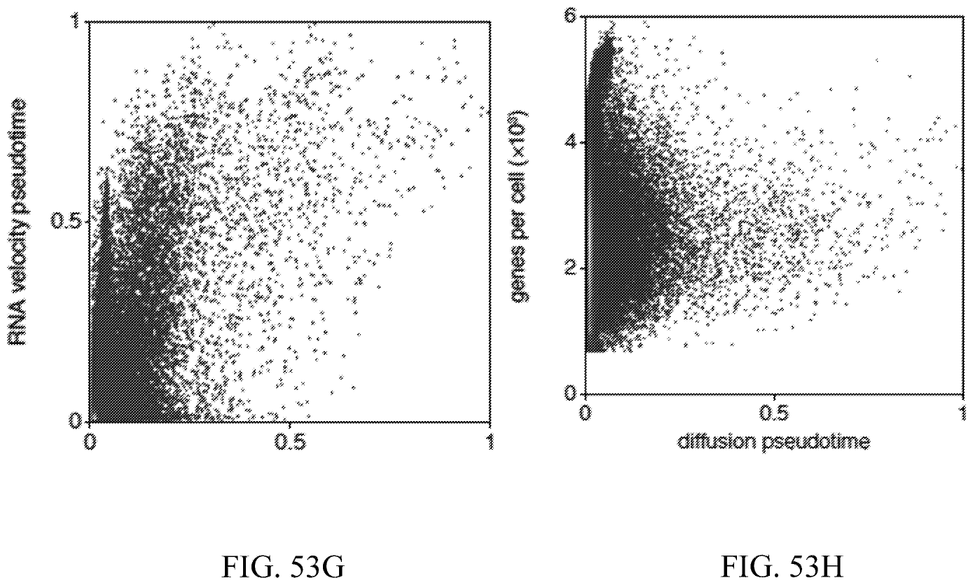

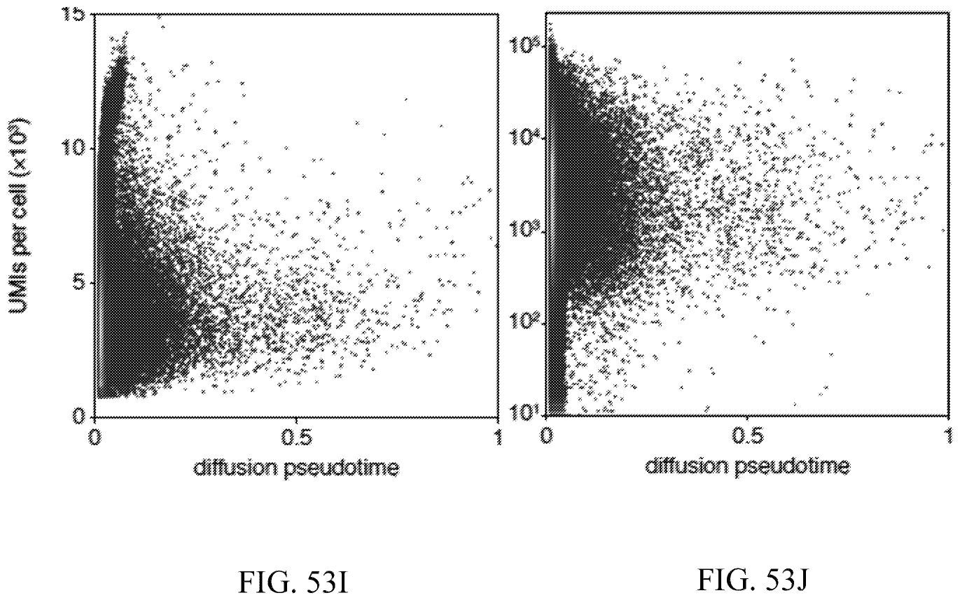

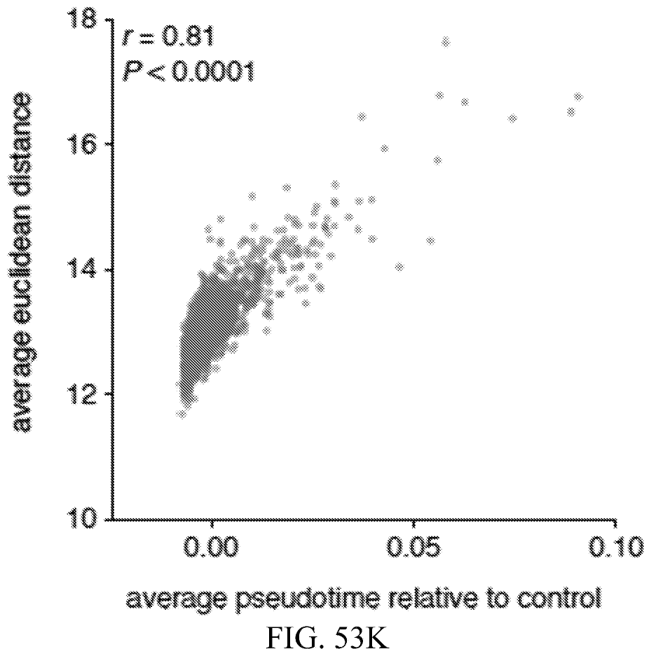

Over a quarter of TFs direct differentiation of hESCs. To study the effects of TF overexpression on hESC differentiation, Applicants computationally inferred differentiation trajectories from the TF Atlas. Applicants used two different approaches to order TF-overexpressing cells in pseudotime based on expression profile similarity to cells expressing GFP or mCherry controls (FIG. 42C, D, FIG. 52F, G, and FIG. 53A-F). Inferred pseudotimes were comparable between the two methods and correlated with the scale of expression changes, rather than quality control variables such as the number of detected genes per cell (FIG. 53G-K).

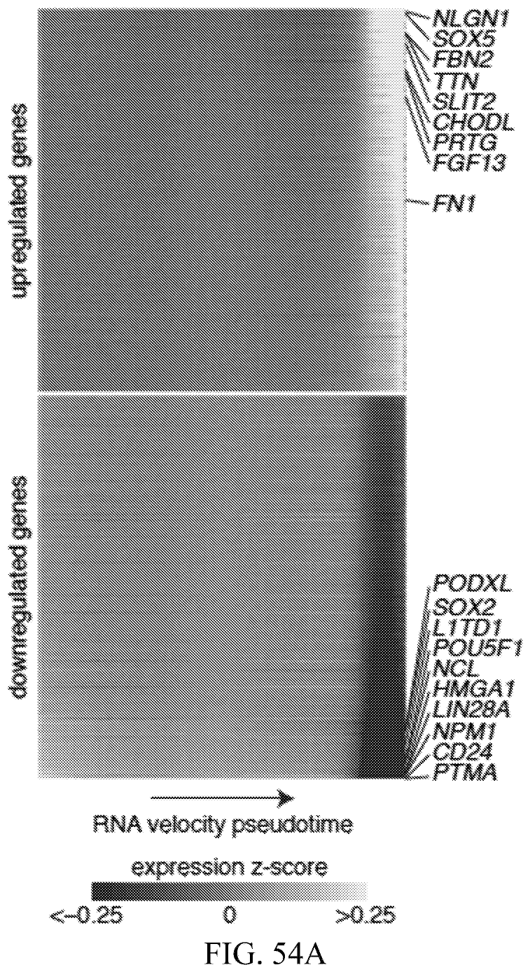

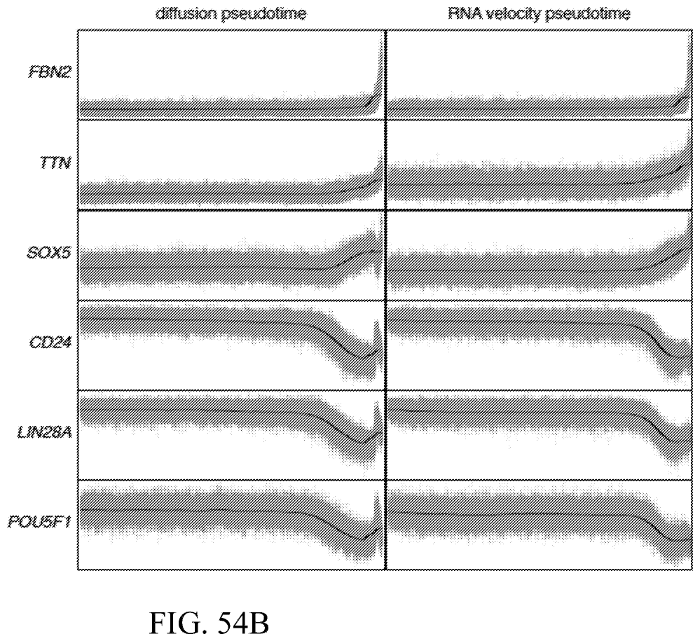

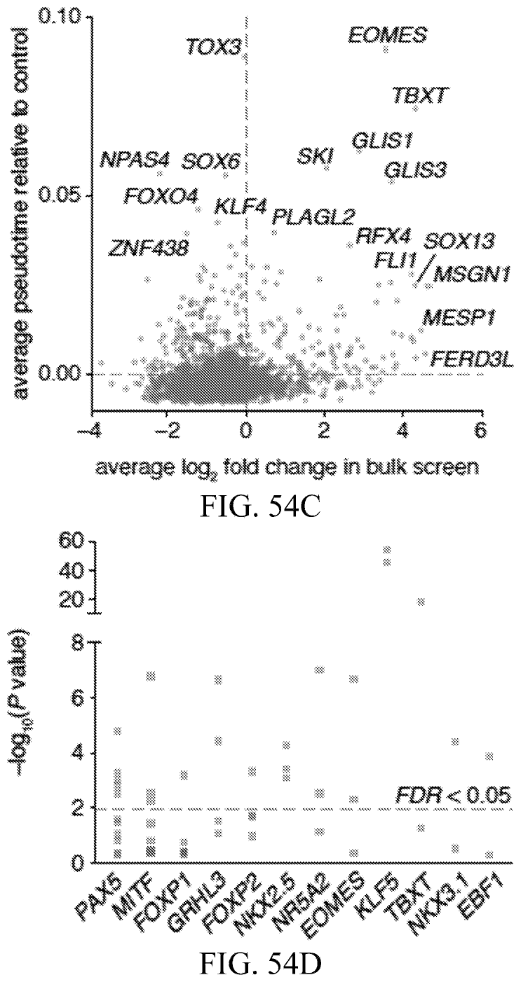

Confirming Applicants' pseudotime inference, genes that drive differentiation (FBN2, TTN, and SOX5) were upregulated over pseudotime, whereas those that maintain pluripotency (CD24, LIN28A, and POU5F1 (OCT4)) were downregulated (FIG. 42E, FIG. 54A, B, and Table 16). Accordingly, pathway analysis confirmed that differentiation pathways such as axonogenesis and heart morphogenesis were enriched in pseudotime-upregulated genes (FIG. 42F). Pluripotency maintenance pathways such as telomere and stem cell maintenance were enriched in pseudotime-downregulated genes (FIG. 1G). Besides pluripotency maintenance, translation was by far the most significantly downregulated pathway with increased pseudotime, as the majority of top downregulated genes were ribosomal genes and translation initiation factors (FIG. 42G). This may suggest that regulation of either translation (41) or cell growth (42) could play a major role in differentiation.

Using pseudotime as a measurement of differentiation, Applicants evaluated the ability of each TF isoform to direct differentiation by comparing the pseudotime distribution of cells with each TF to those of control cells (Wilcoxon rank-sum test). Some TF ORFs that increased pseudotimes were also enriched in the differentiated cells from the pooled marker-based TF screen (TBXT, MSGN1, RFX4, and EOMES), while others (SOX6, KLF4, and TOX3) were only enriched in the scRNA-seq screen, potentially because scRNA-seq captures the full expression profile, rather than only two pluripotency marker genes (FIG. 54C). Surprisingly, 496 (27%) TFs encoding 694 (20%) isoforms could significantly alter pseudotime (FDR<0.05), suggesting a high percentage of TFs could act as master regulators, perhaps because of the relatively open chromatin in hESCs (43). Notably, differentiation efficiencies were sometimes drastically different between splice isoforms of the same TF gene (FIG. 42H and FIG. 54D). Applicants could not simply identify the splice isoform that most efficiently induced differentiation based on nominal protein domain annotations, length, or consensus sequence (Table 17), highlighting the need to experimentally test different isoforms.

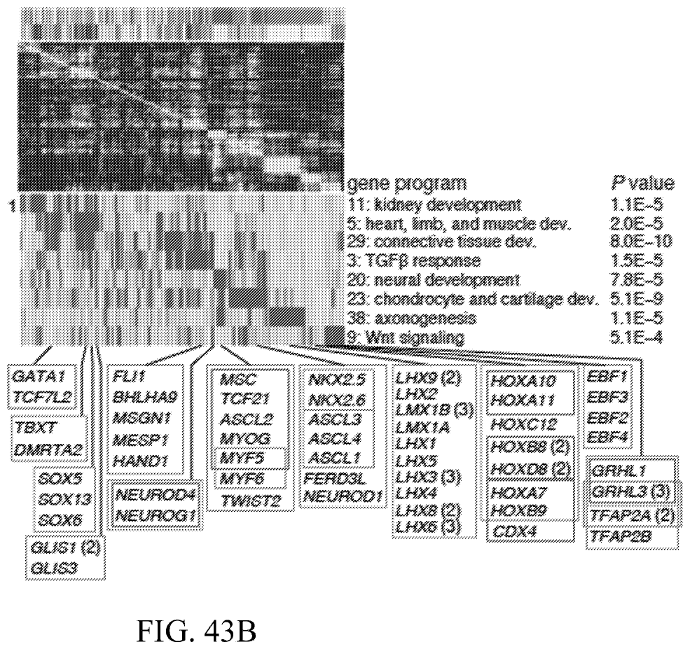

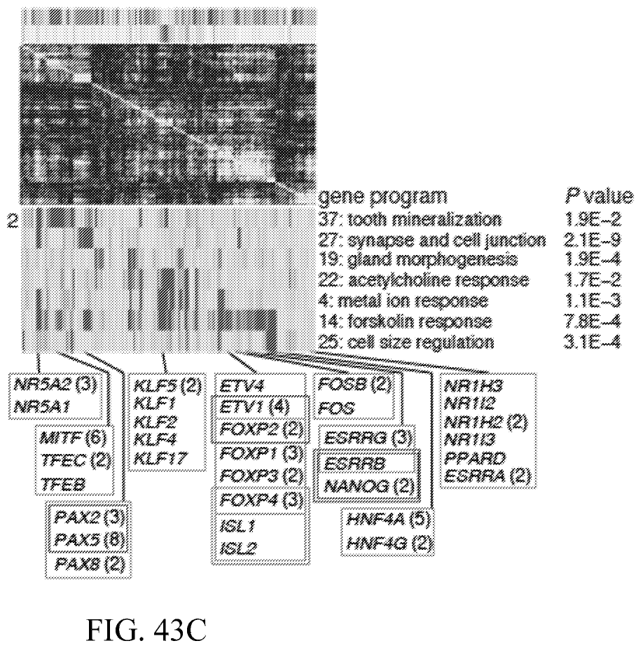

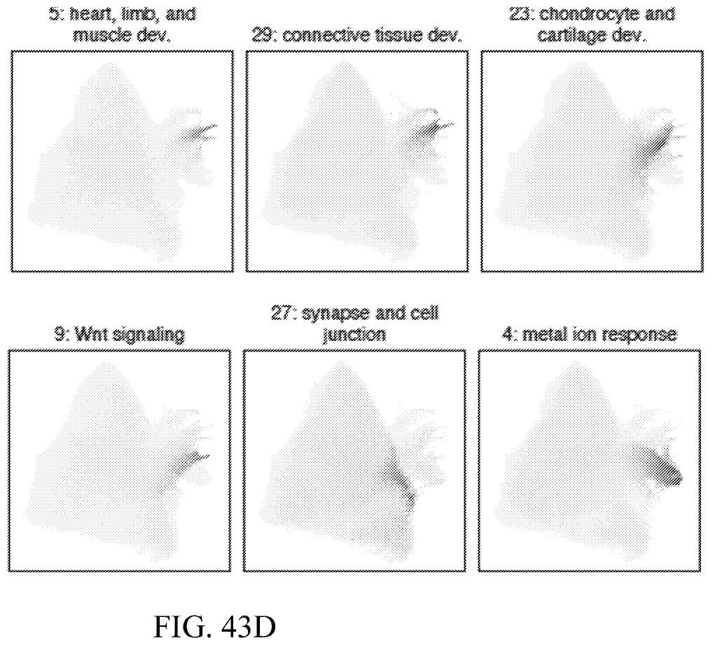

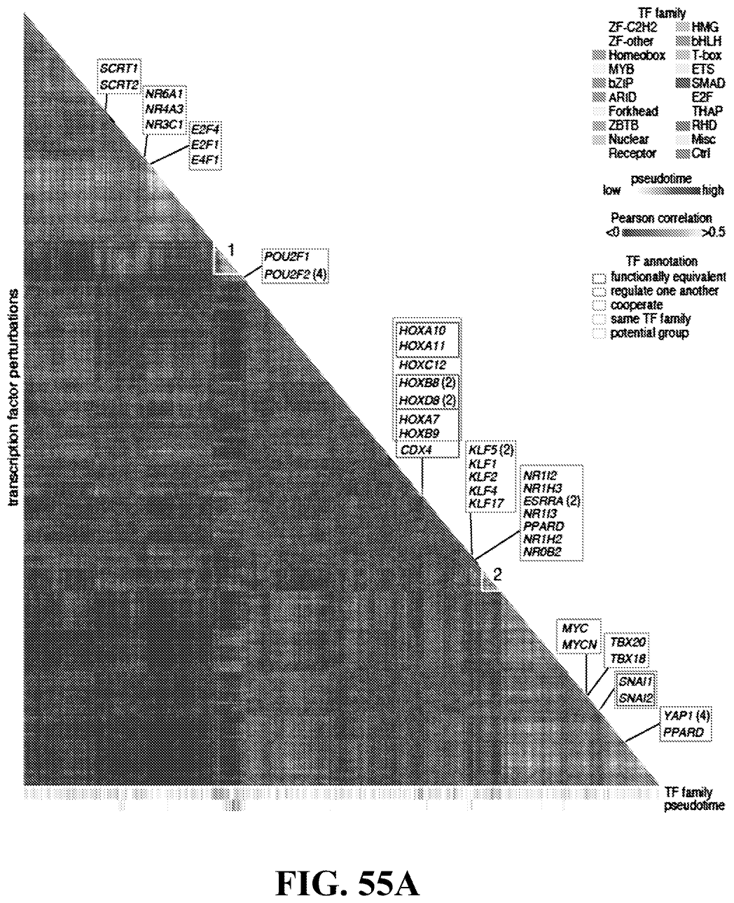

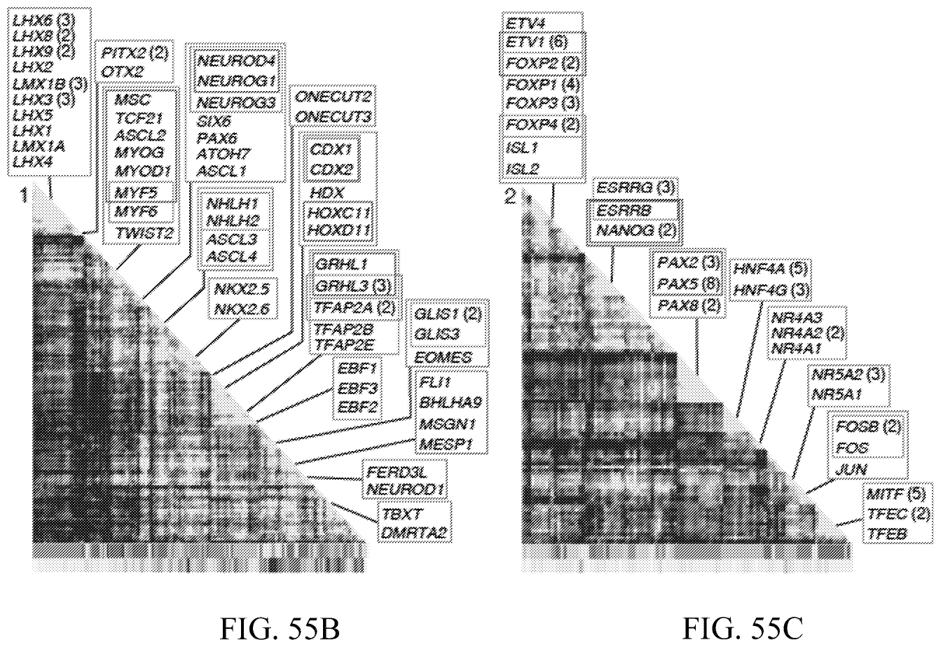

Co-functional TF modules annotate uncharacterized TFs. Applicants next leveraged the comprehensive scope of Applicants' TF Atlas to group co-functional modules of TFs that impact the same programs, and thus classify unknown, orphan TFs. Applicants first inferred gene programs across the mean expression profiles associated with each TF using non-negative matrix factorization (NMF) and then clustered the 3,266 TFs by their effects across the programs (clusters have a maximum Pearson correlation P-value of 10-7; FIG. 43). Clustering TFs using pairwise correlation of their mean expression profiles produced similar groupings (FIG. 55).

Our analysis grouped together splice isoforms and TFs that are known to be functionally equivalent (e.g., NEUROD4 and NEUROG1 (44), PAX2 and PAX5 (45), ESRRB and NANOG (46)) (FIG. 43B, C), as well as TFs from the same family, including 18 TF isoforms from the Lim homeobox TF family (LHXs 1-6, 8, 9 and LMXs 1A and 1B), 9 posterior Hox genes (HOXA7, HOXB8, HOXD8, HOXB9, HOXA10, HOXA11, and HOXC12), and 8 nuclear receptors (NR1H2, NR1H3, NR112, NR113, PPARD, and ESRRA) (FIG. 43B, C).

This analysis helps annotate relatively uncharacterized TFs by their association with well-characterized TFs in the same co-functional module. For instance, there is little functional information on KLF17, and it is considered distantly related to the rest of the KLF TF family, members of which can function as activators or repressors (47). As KLF17 induces a similar gene program to KLF activators (KLFs 1,2,4, and 5), it is likely an activator (FIG. 43C). TFs from different families also group together based on similarities in gene programs, such as DMRTA2 and TBXT in mesoderm development and FERD3L and NEUROD1 in neural development (FIG. 43B). These results demonstrate the utility of the TF Atlas for identifying shared functional modules of TFs.

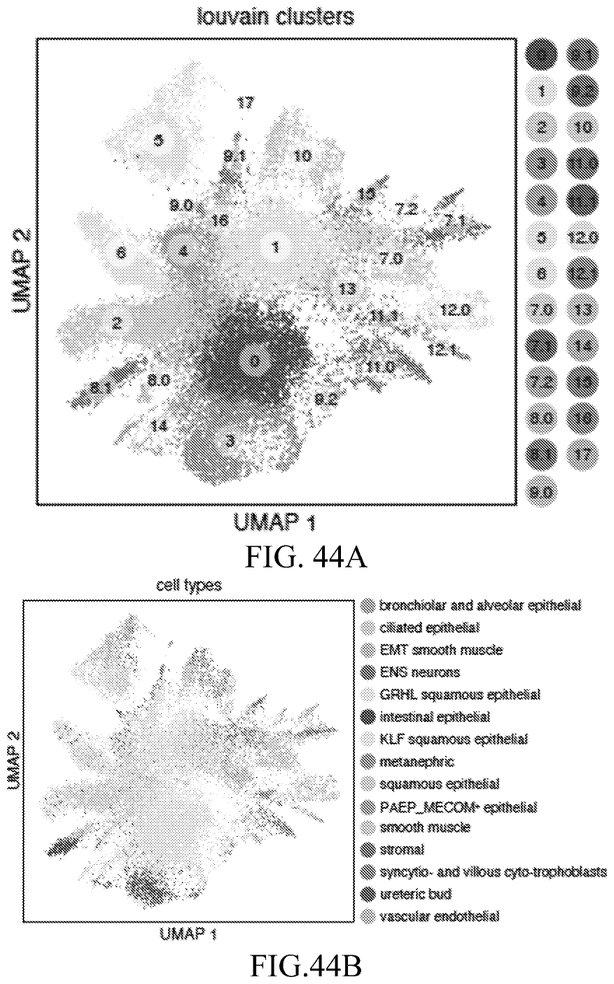

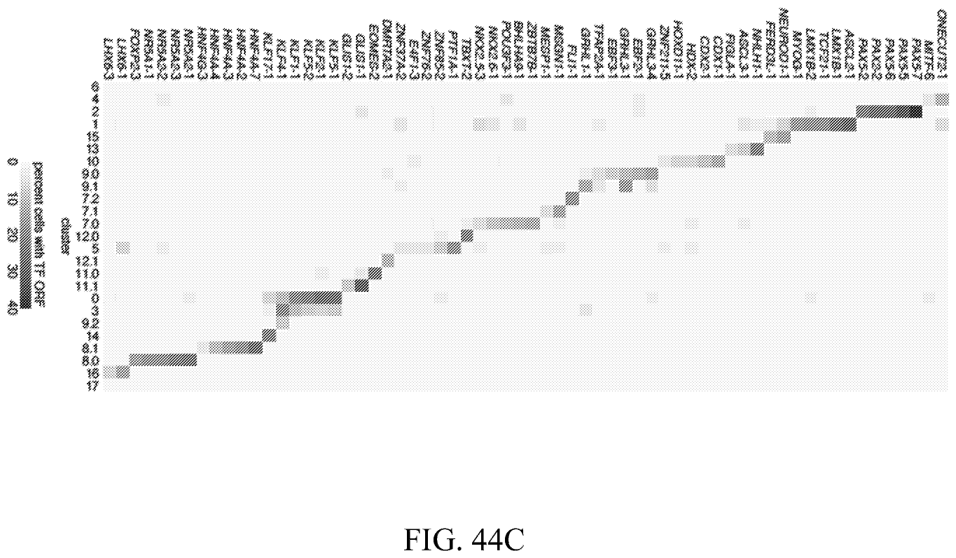

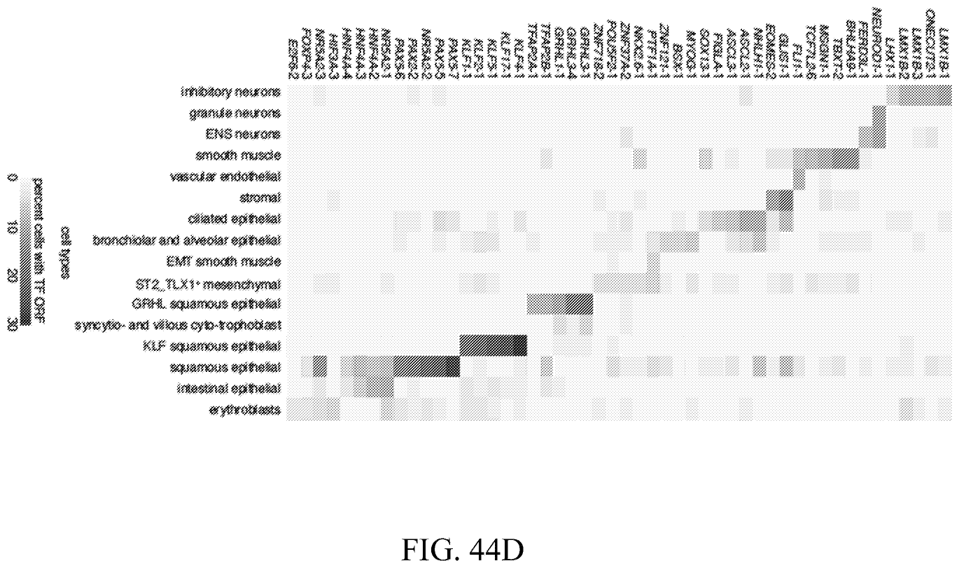

Mapping TF effects to reference cell types. To characterize the ability of TFs to drive differentiation to particular endpoints, Applicants next mapped TF-induced expression profiles to those of reference cell types. Applicants subclustered differentiated cells from the TF Atlas (clusters 6-8 from FIG. 42B and FIG. 53A, defined by differentiation pseudotime, FIG. 42D and FIG. 53B; see Methods) to obtain higher resolution and annotated each cell in Applicants' atlas by label-transfer from the human fetal transcriptome atlas (48) cell type that most closely resembled it (FIG. 44A, B).

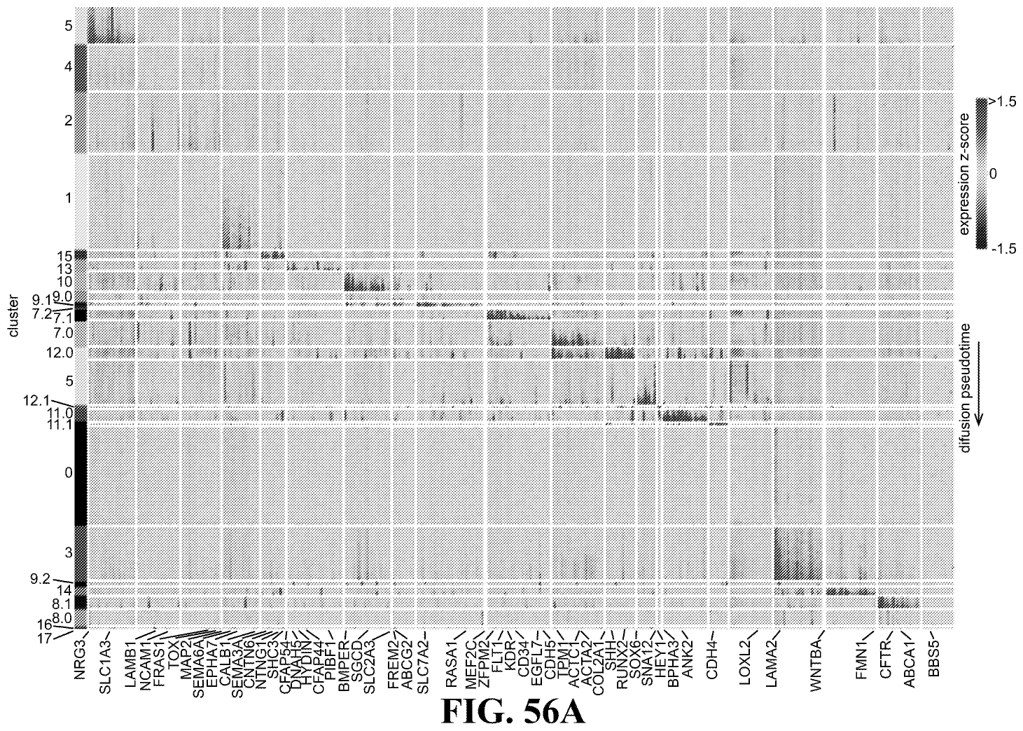

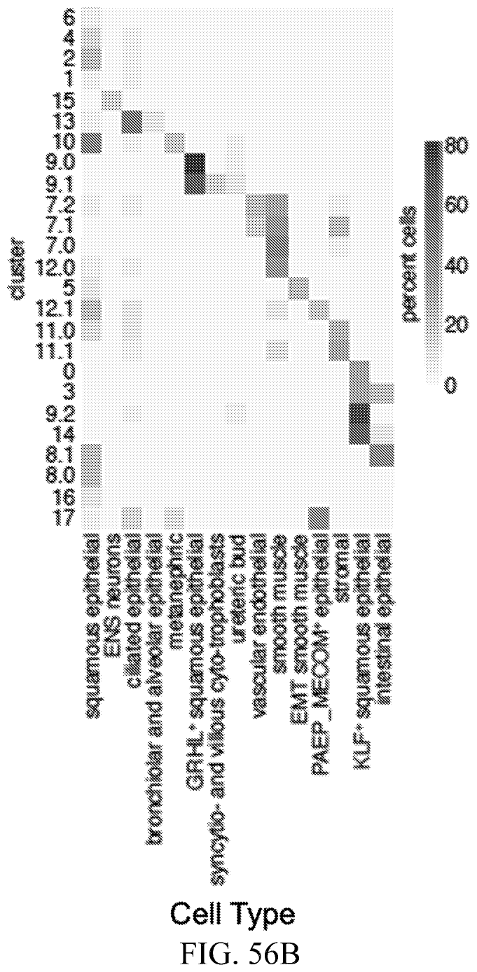

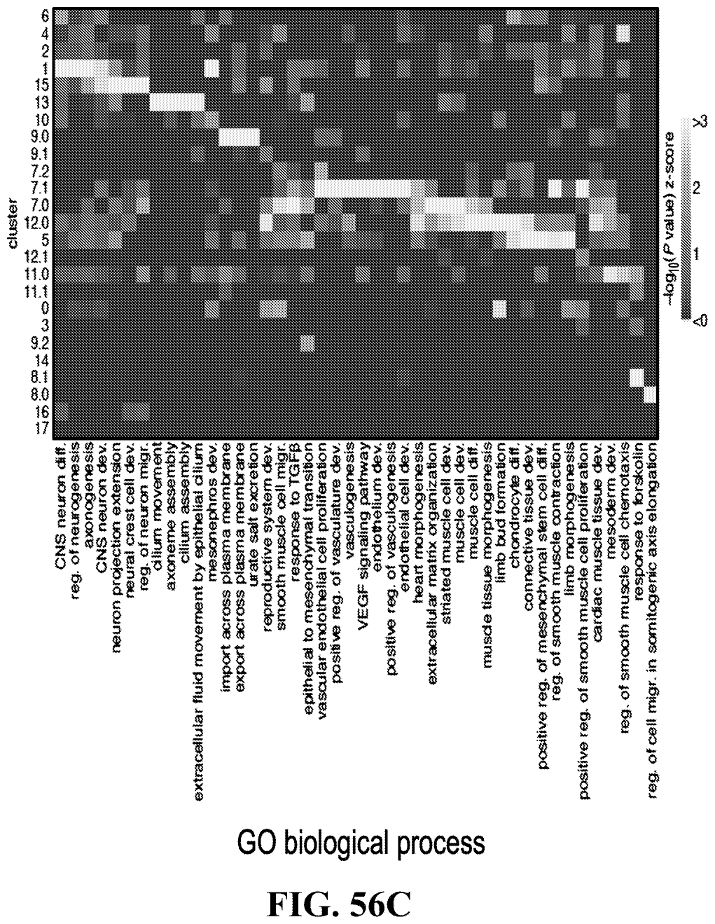

The mapping results suggest that Applicants generated cells resembling types from each of the three germ layers, such as (i) squamous epithelial and neurons from ectoderm, (ii) smooth muscle and metanephric from mesoderm, and (iii) intestinal epithelial and bronchiolar and alveolar epithelial from endoderm, as well as from the extraembryonic lineage (syncytio- and villous cyto-trophoblast) (FIG. 44B). Each cluster is comprised of cells with distinct groups of TF ORFs (adjusted mutual information score of 0.43 for TFs with >5% cells in any cluster) and is associated differentially expressed genes, indicating the diversity and specificity of TF-induced gene programs and differentiation states, as well as high penetrance of TF effects (FIG. 44C, FIG. 56A, and Table 18). The biological pathways enriched in each cluster were consistent with their assigned cell type annotations (FIG. 56B, C). For instance, cluster 13 was enriched in cilium assembly and movement pathway genes and mapped to ciliated epithelial cells. Cluster 7.1 was enriched in vasculogenesis and vasculature development pathway genes and mapped to vascular endothelial cells.

Matching TF ORFs to cell types suggested candidate TFs that could induce differentiation of each cell type (FIG. 44D). Notably, several of these candidate TFs are known to be important for specifying the target cell type during development, further supporting the mapping results. For instance, FERD3L is important for neurogenesis (49), FLI1 for endothelial development (50), and KLF4 for intestinal epithelial homeostasis (51, 52).

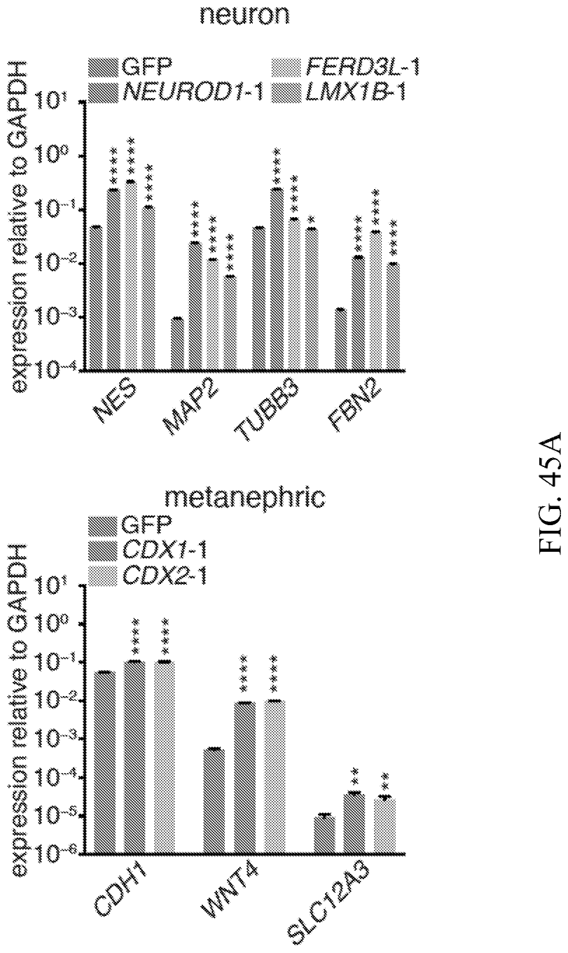

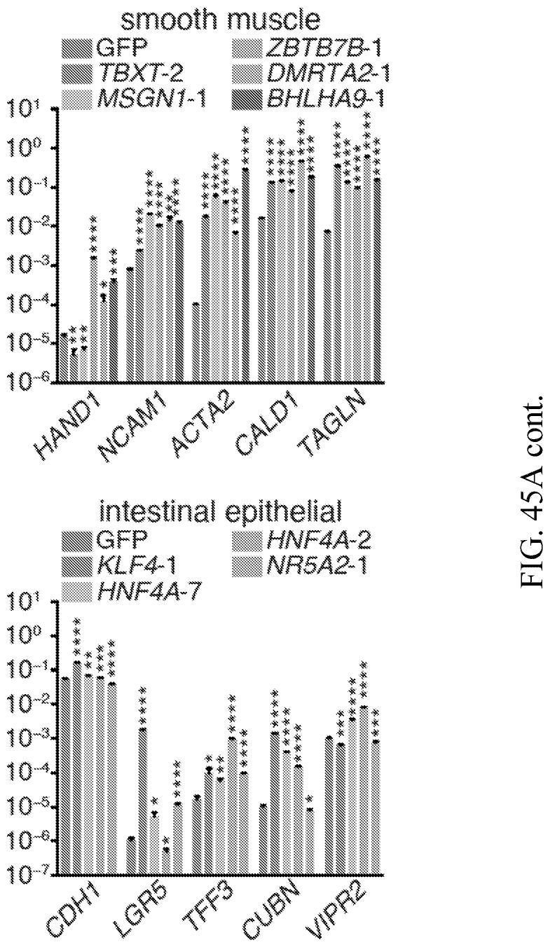

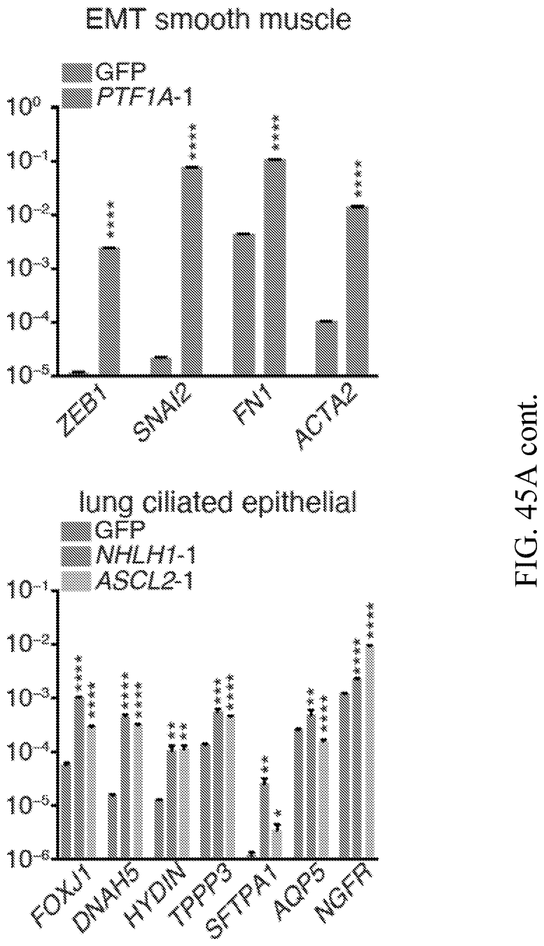

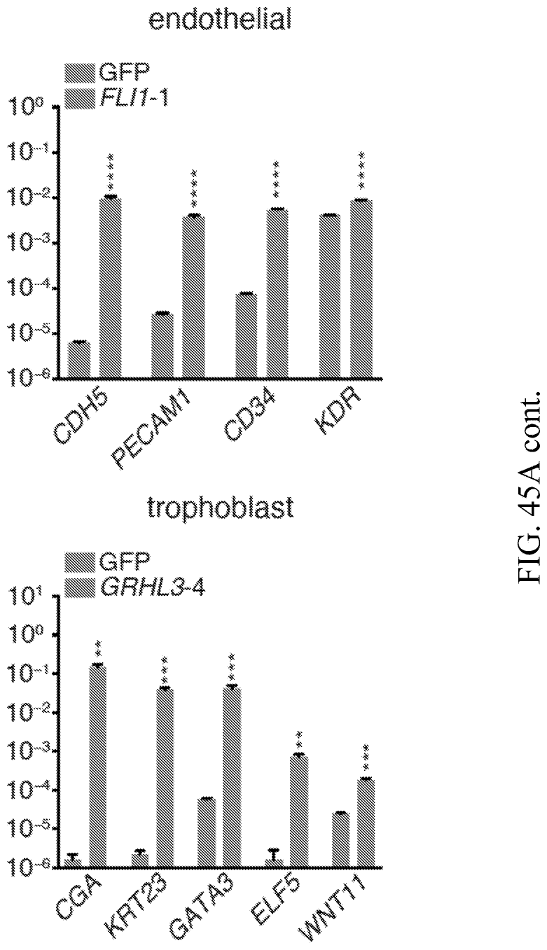

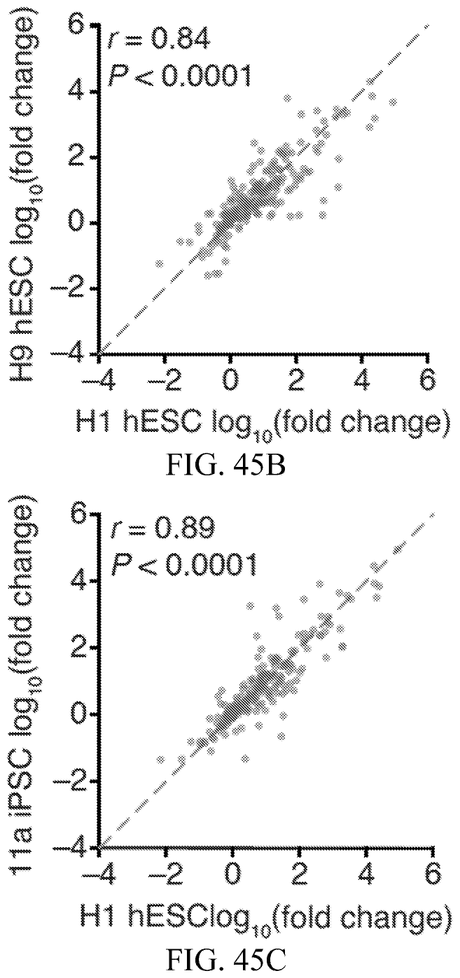

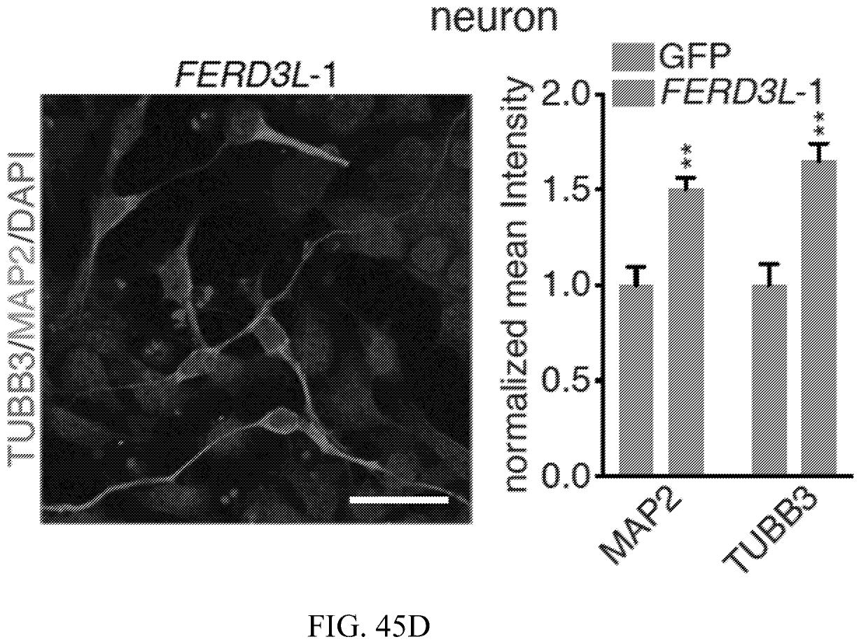

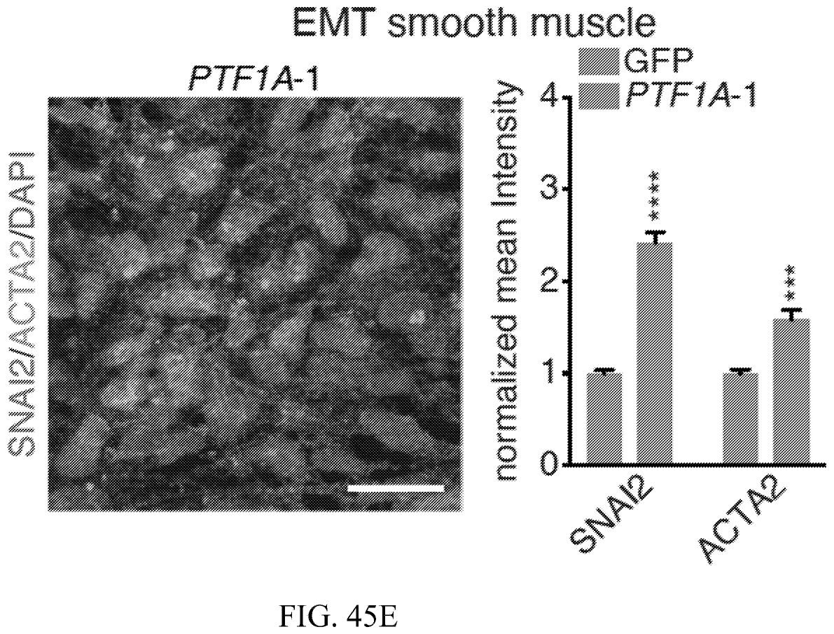

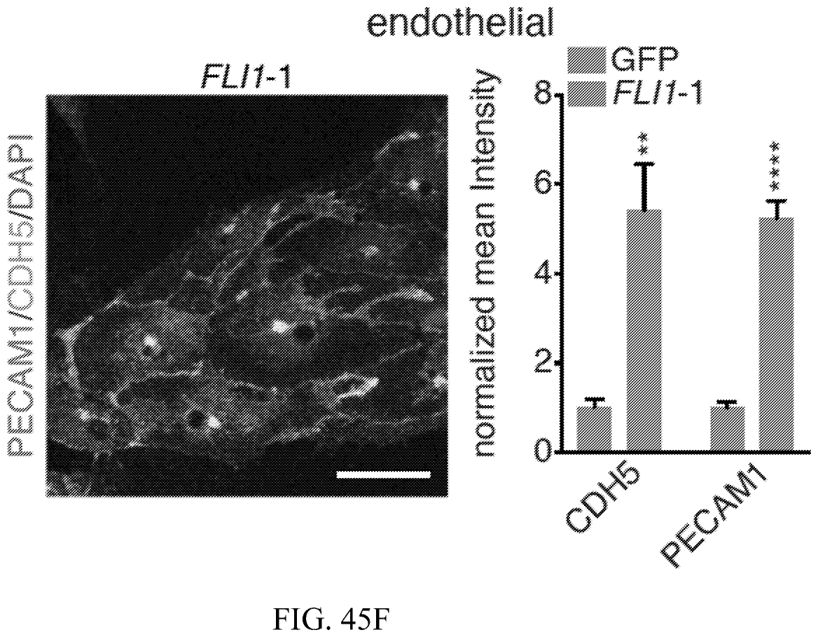

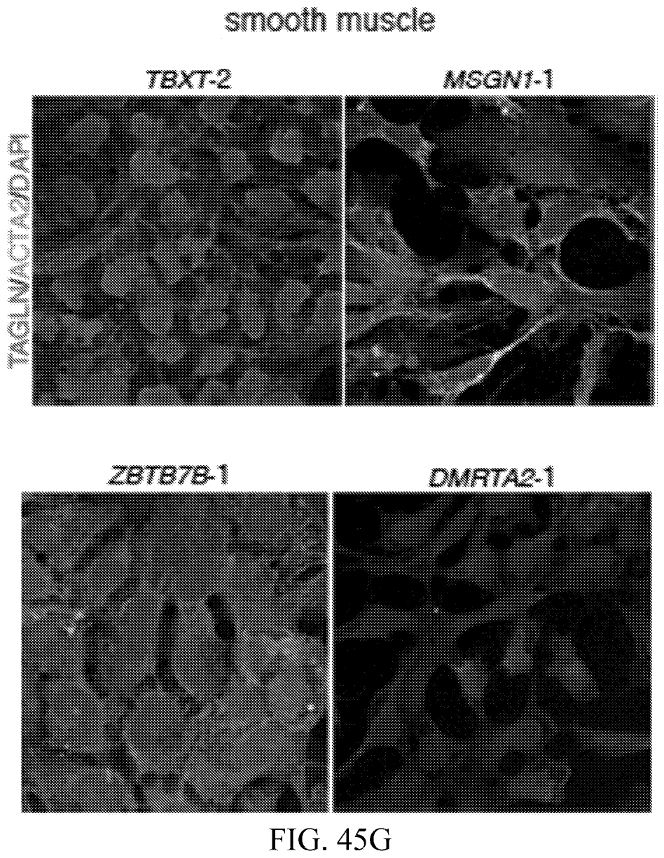

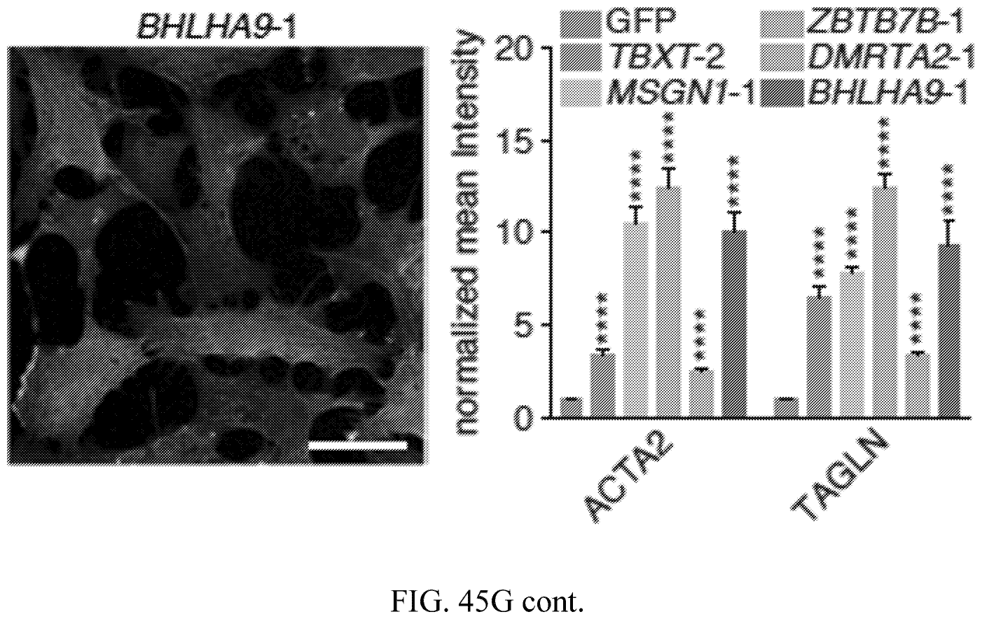

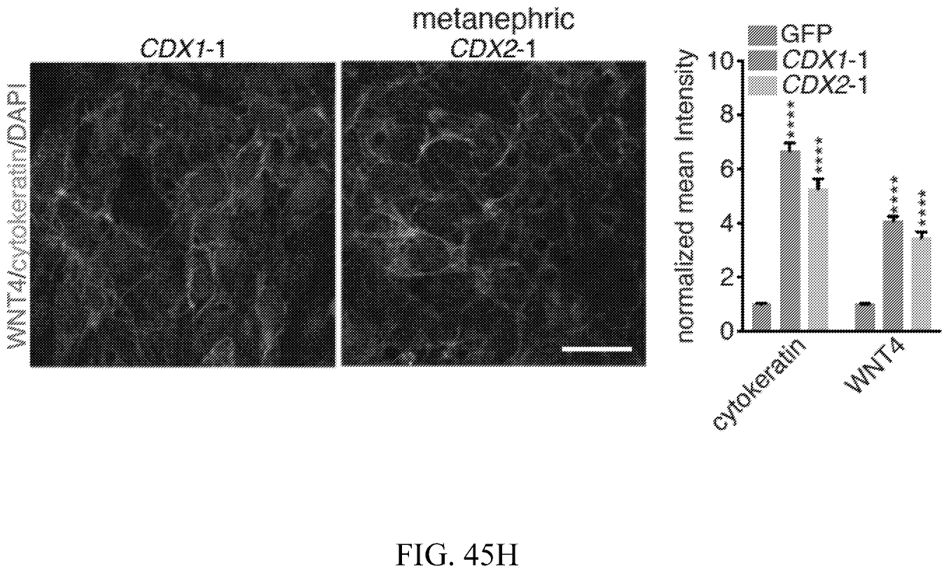

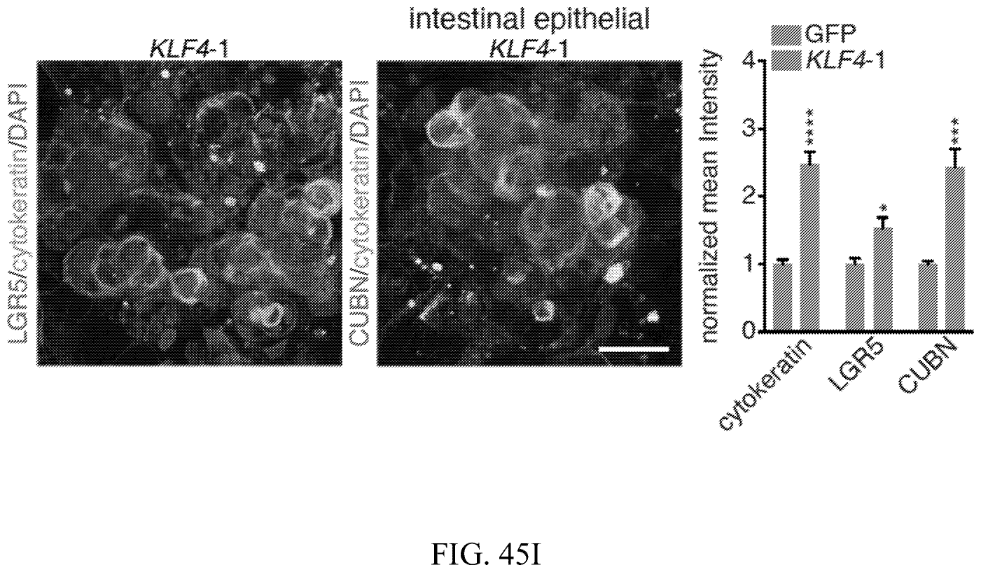

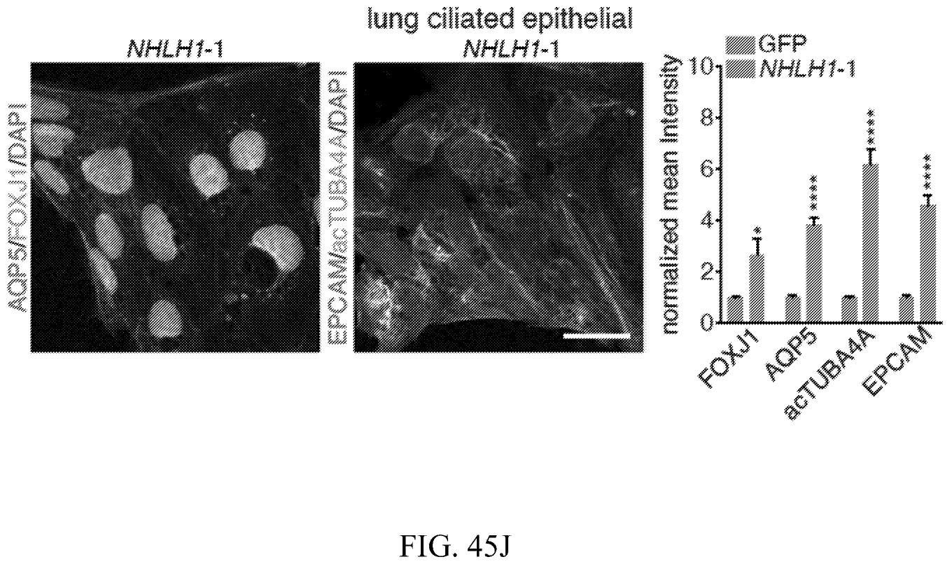

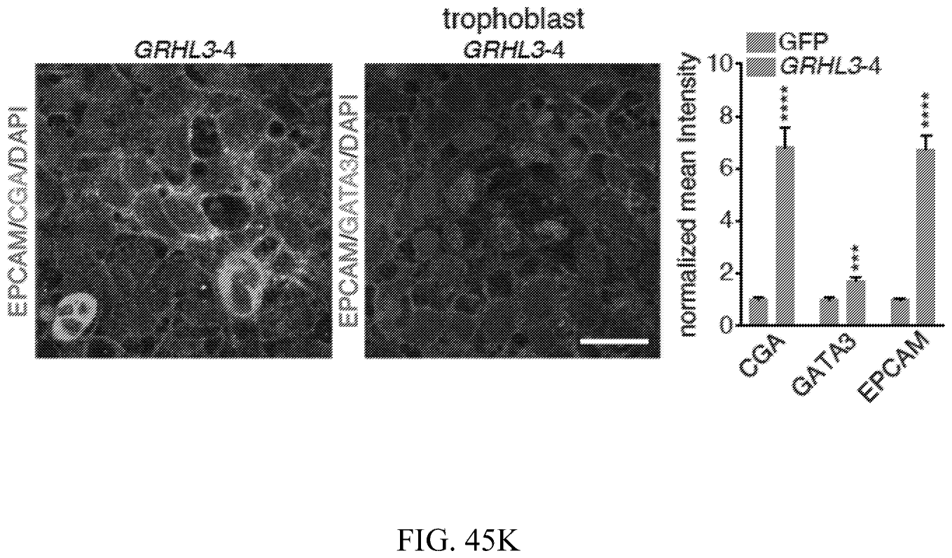

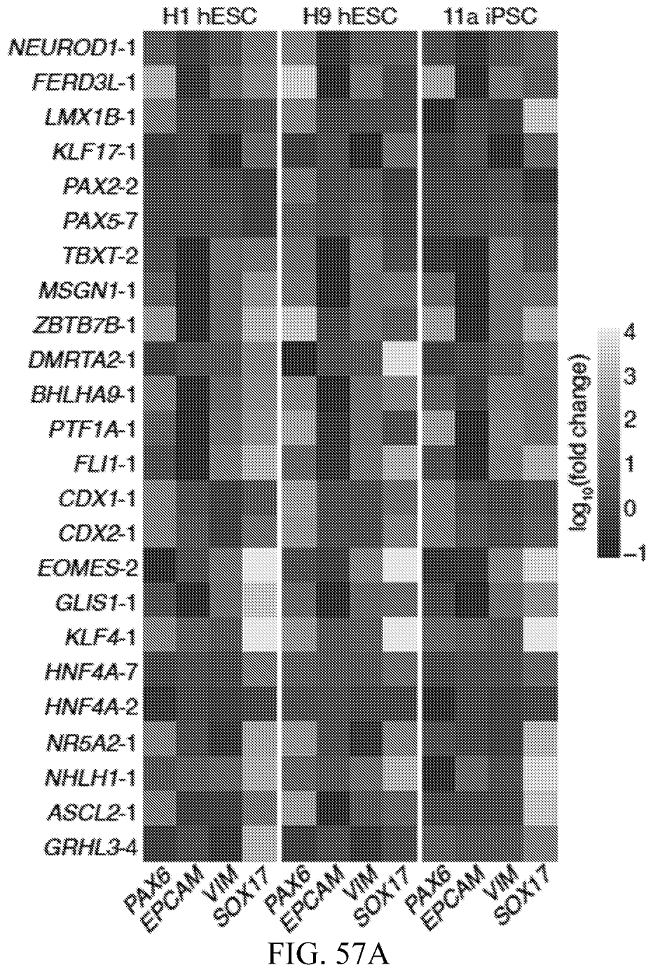

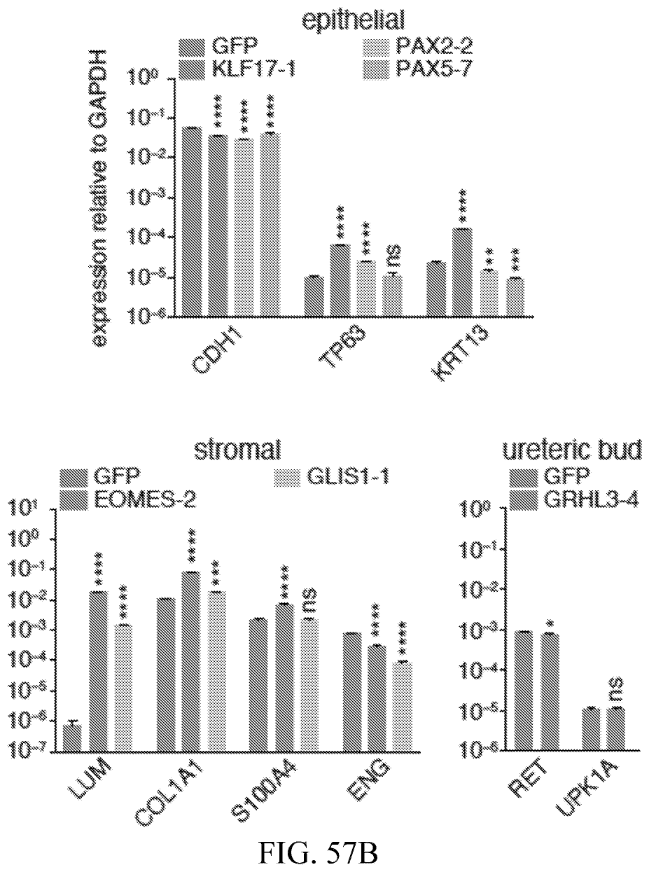

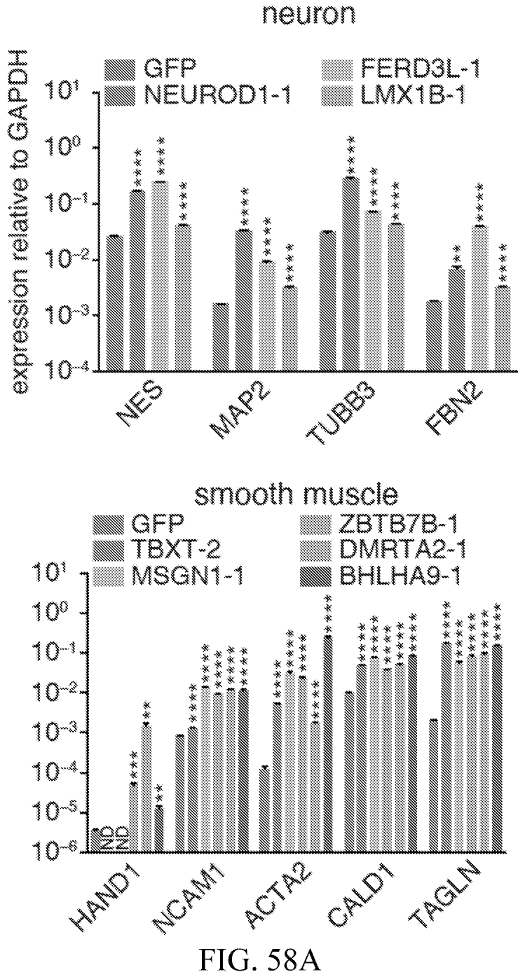

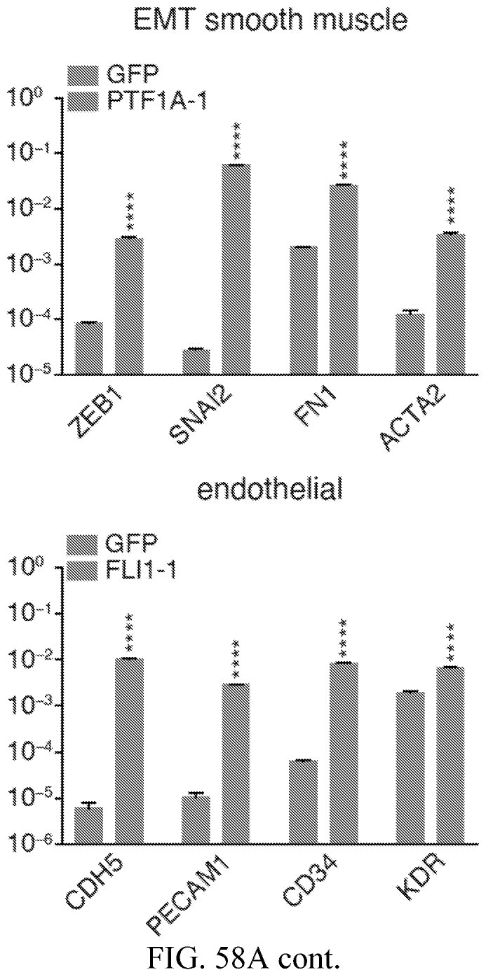

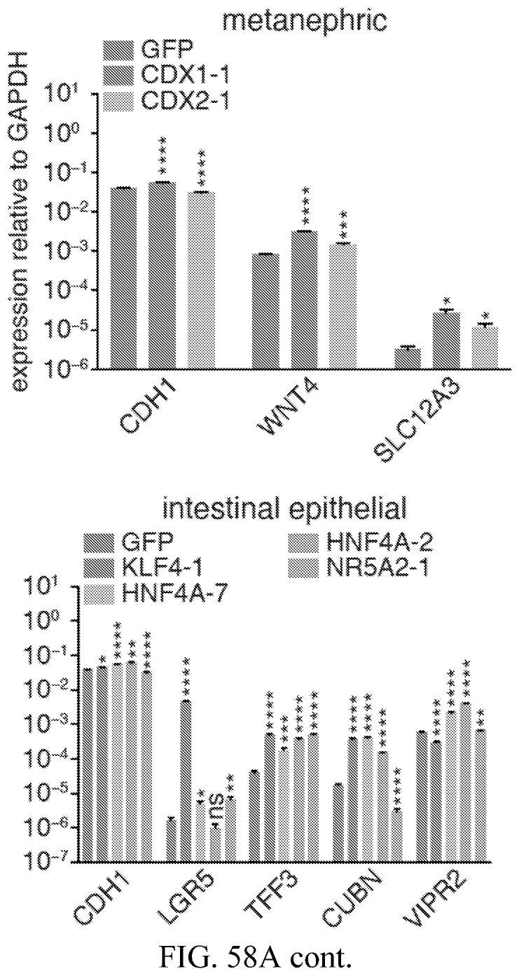

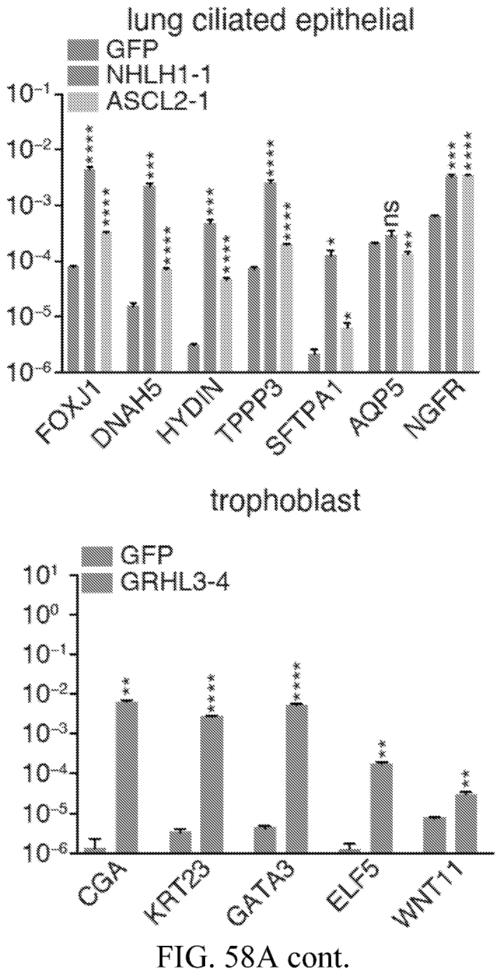

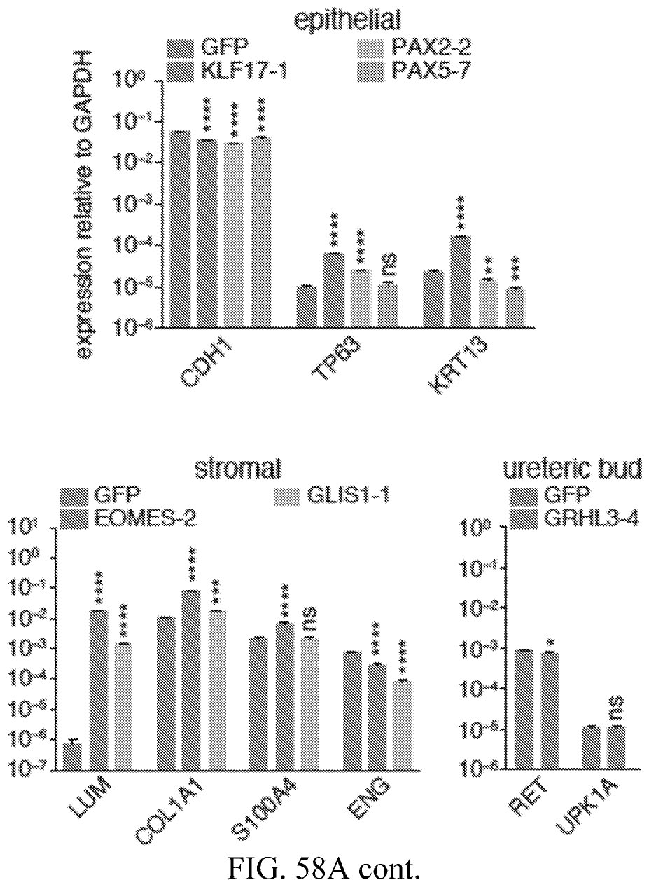

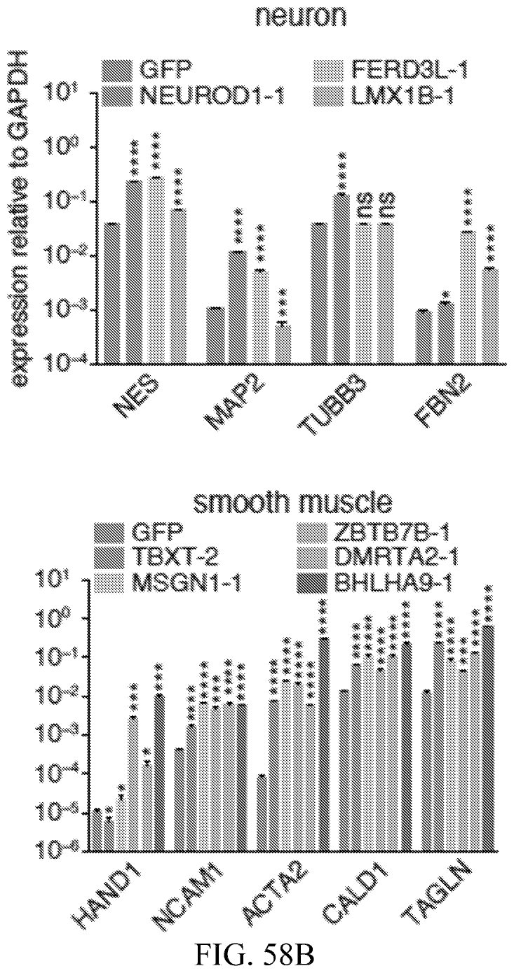

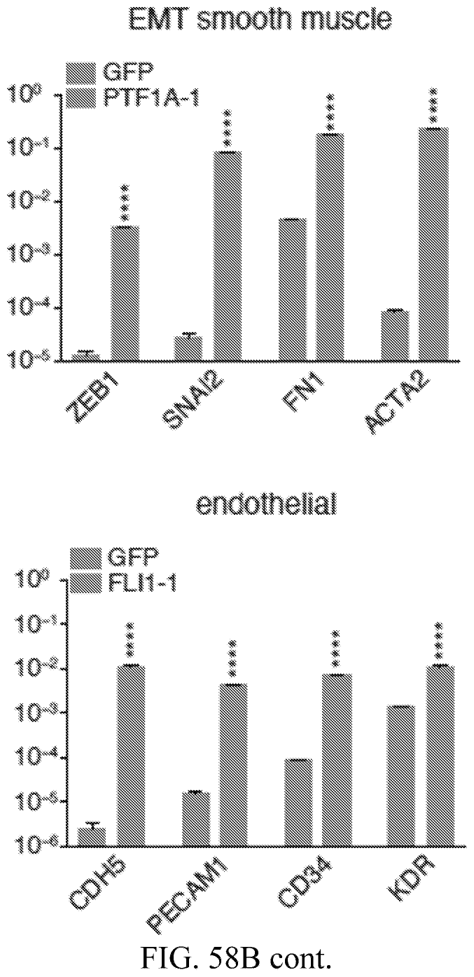

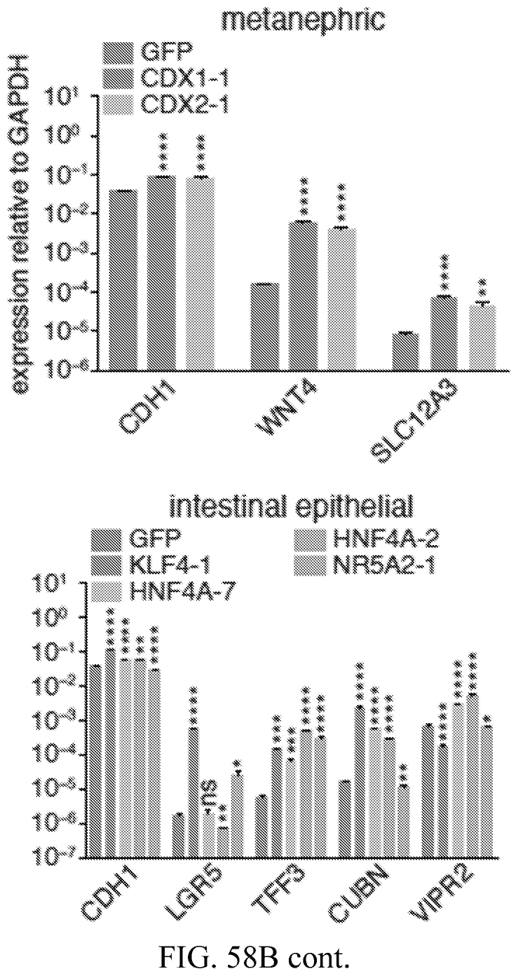

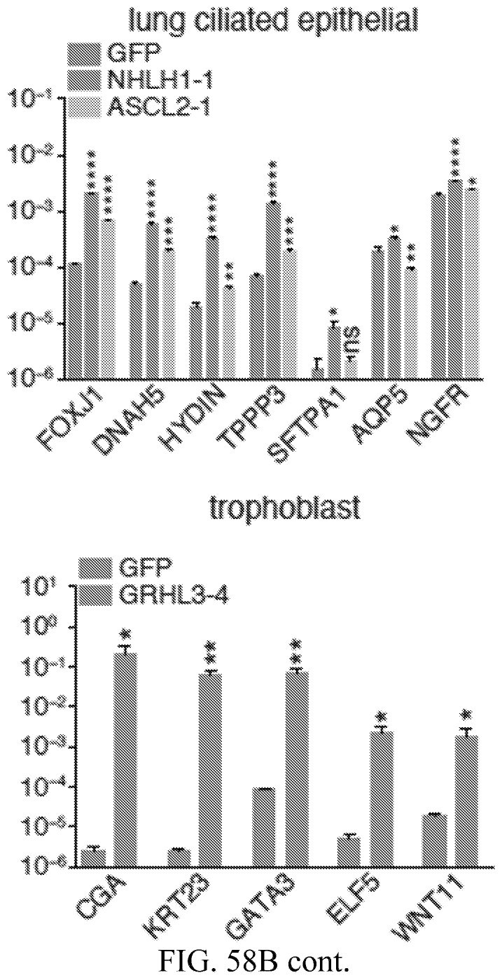

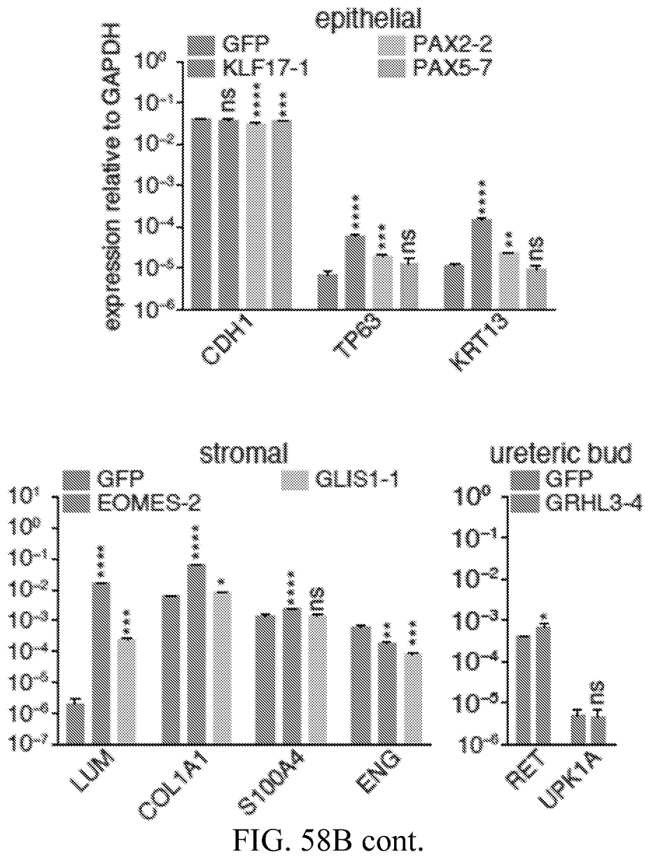

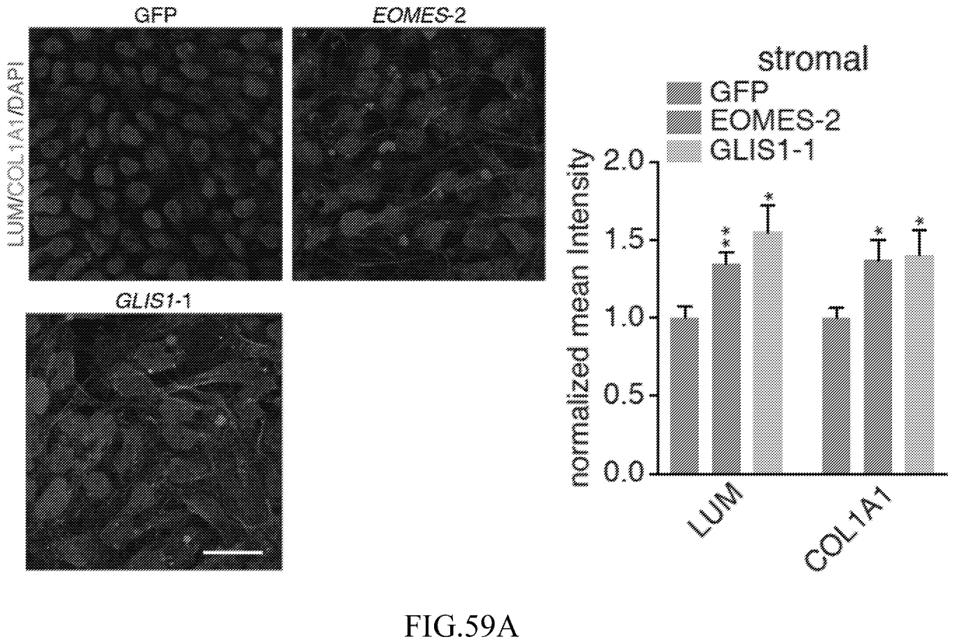

Validation of differentiation-directing TFs. To validate the cell type mapping results, Applicants selected a diverse set of 24 candidate TFs that were predicted to generate 10 distinct cell types. Out of 24 candidate TFs, only one, NEUROD1, has been previously shown to direct differentiation of the nominated cell type and was included as a control (28). Three pairs of TFs that induce similar gene programs (CDX1 and CDX2, PAX2 and PAX5, and two isoforms of HNF4A) were included for comparison. Applicants overexpressed each candidate TF separately in H1 hESCs for 7 days and measured expression of known marker genes that delineate each cell type (FIG. 45A, FIG. 57). Most candidate TFs (22 out of 24) induced expression of marker genes for the cell type predicted by Applicants' TF atlas screen and analysis (FIG. 45A and FIG. 57). For instance, based on marker expression, NEUROD1, FERD3L, and LMX1B produced peripheral neuron-like cells; FLI1 produced vascular endothelial-like cells; KLF4, HNF4A, and NR542 produced intestinal epithelial-like cells; and NHLH1 and ASCL2 produced lung ciliated epithelial-like cells. Within each cell type, different candidate TFs sometimes generated distinct mean expression profiles of marker genes (FIG. 45A), indicating differences in either differentiation efficiencies or trajectories towards the target cell type. GRHL3, which was predicted to induce both trophoblasts and ureteric bud cells, only generated trophoblast-like cells (FIG. 45A and FIG. 57B). Overexpression of EOMES and GLIS1 increased expression of LUM and COL1A1, but not ENG, indicating that the TFs produced general stromal-like cells rather than a subpopulation of ENG-expressing stromal mesenchymal cells (FIG. 57B). Although PAX2 and PAX5 generated distinct expression changes (FIG. 44C), neither produced epithelial-like cells (FIG. 57B). The expression changes induced by each TF were remarkably consistent across two additional cell lines: H9 hESCs and 11a iPSCs (Pearson r=0.84 and 0.89, respectively), suggesting that the TF Atlas results extend beyond the cell line used in the screen (FIG. 45B, C and FIG. 58).

Applicants further validated Applicants' results by immunostaining for a subset of 17 candidate TFs covering 8 cell types, confirming that changes in protein expression and cell morphology were consistent with the target cell type (FIG. 45D-K, FIG. 59). Out of 17 candidate TFs, 15 significantly upregulated protein expression of marker genes and induced morphology that resembled that of reference cell types (FIG. 45D-K, FIG. 59). HNF4A and ASCL2 did not significantly upregulate protein expression of marker genes on average (by automated image quantification): the cells with morphology changes did have increased protein expression, but there was only a low fraction of such differentiated cells with these two TFs (FIG. 59B, C). Together, these results show that Applicants can identify and validate TFs for directed differentiation into diverse cell types.

Targeted TF screening to create tailored cellular disease models. Cellular disease models are a tractable system that can be perturbed, genetically or chemically, to assess effects in a cell type-specific context (6-17). However, it remains challenging or impossible to generate many cell types. To address this challenge, Applicants sought to demonstrate that the MORF library could be applied to create a tailored cellular disease model.

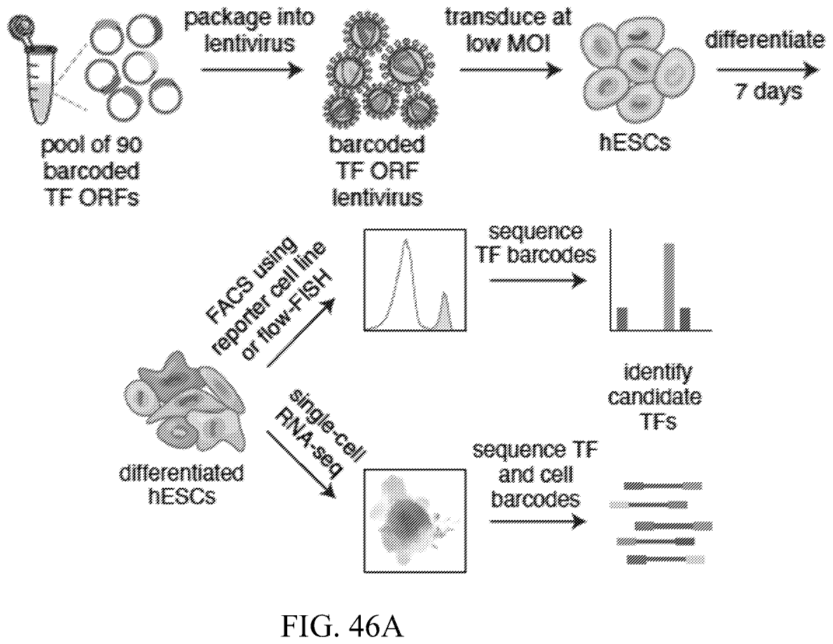

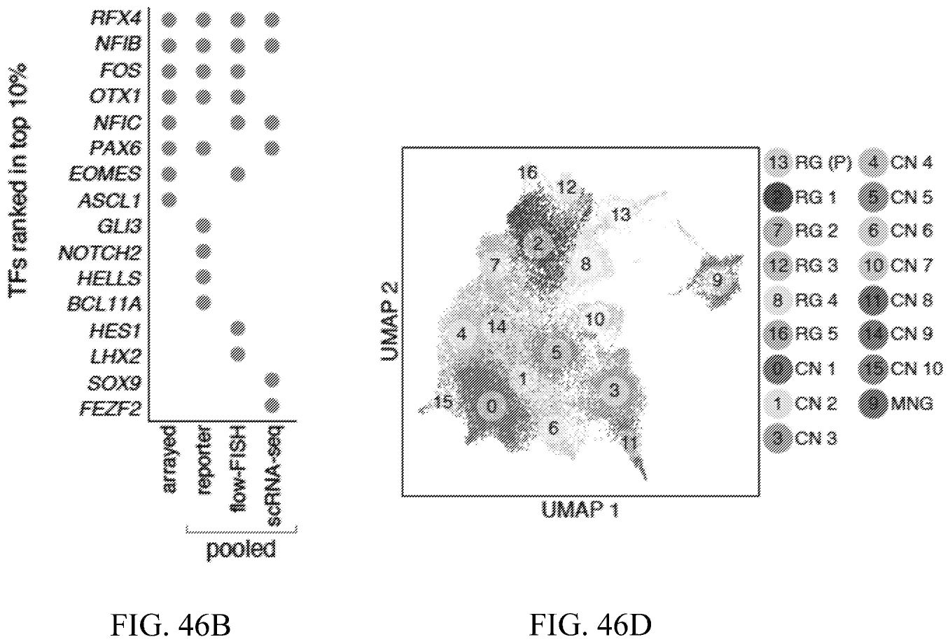

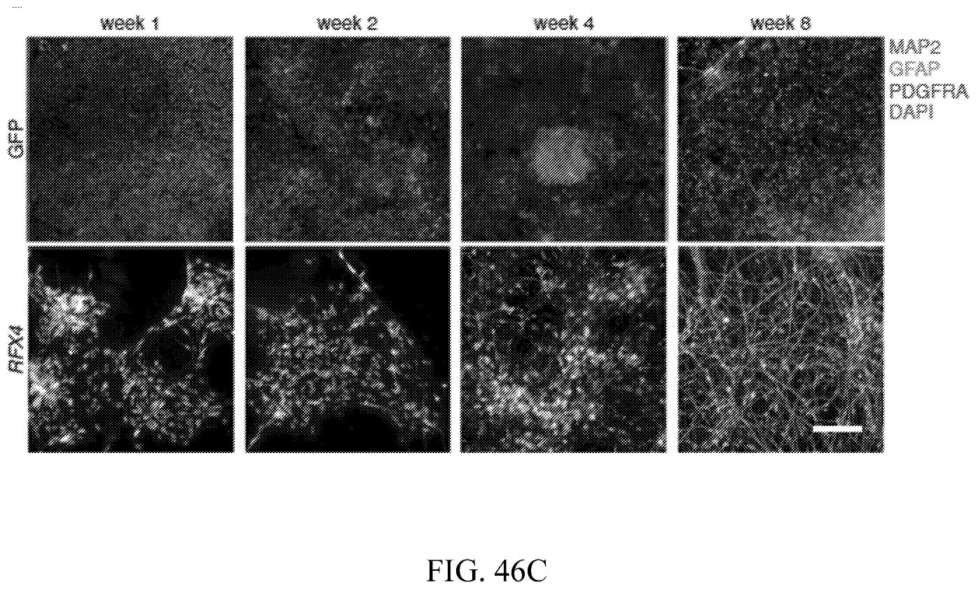

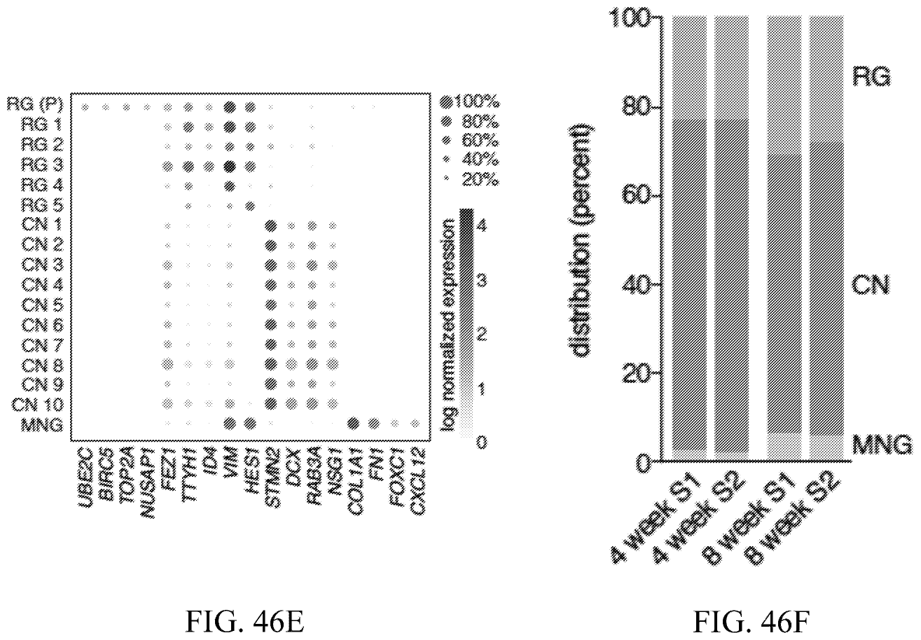

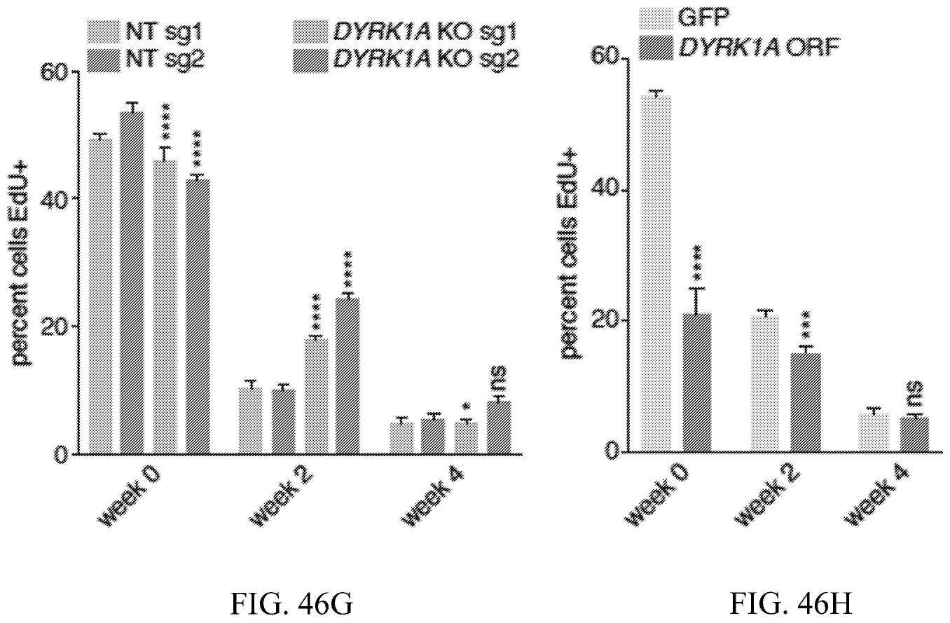

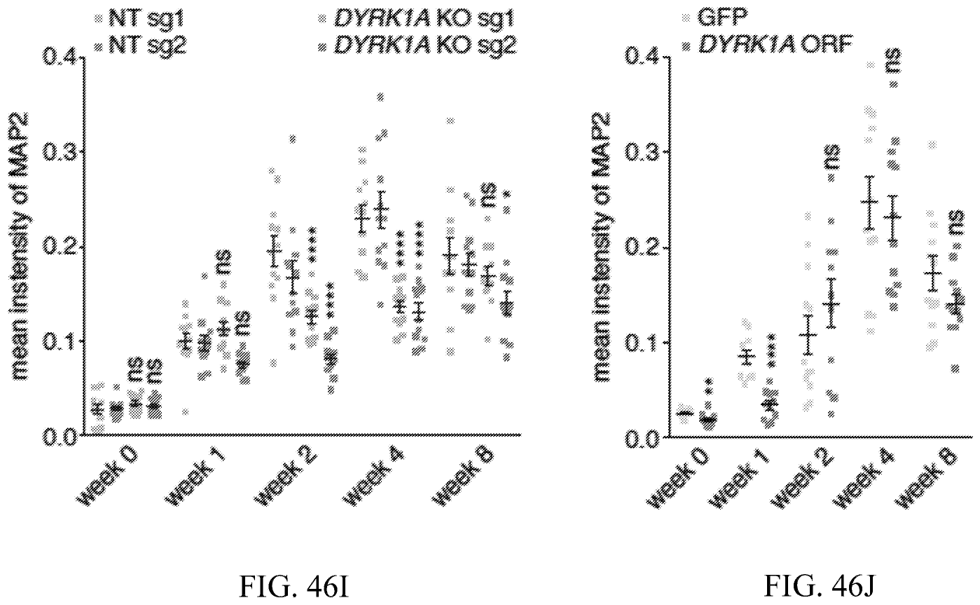

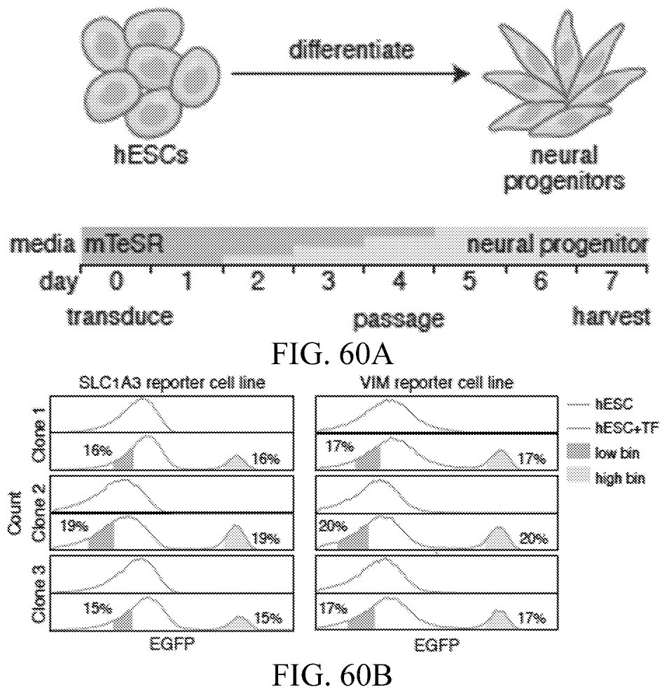

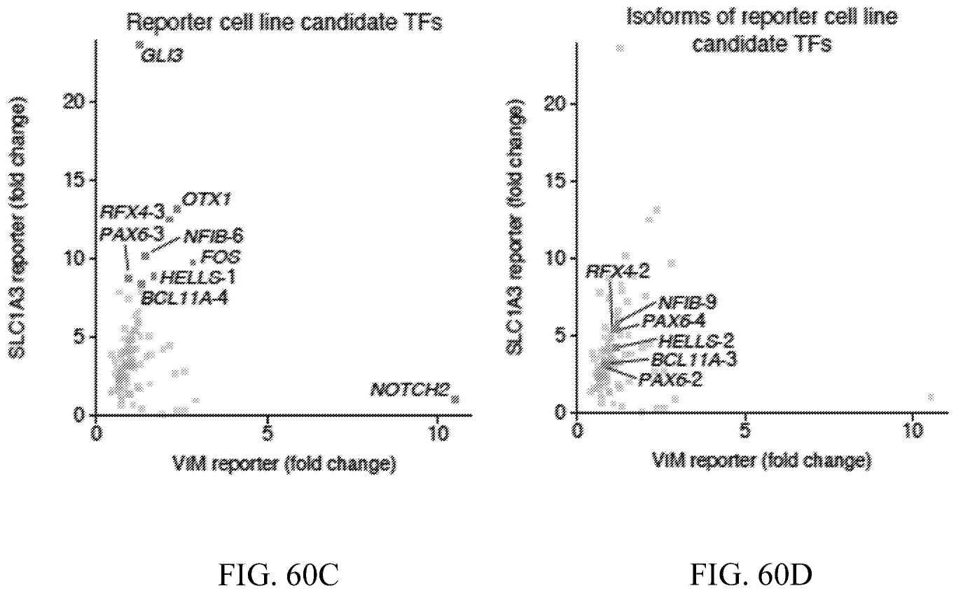

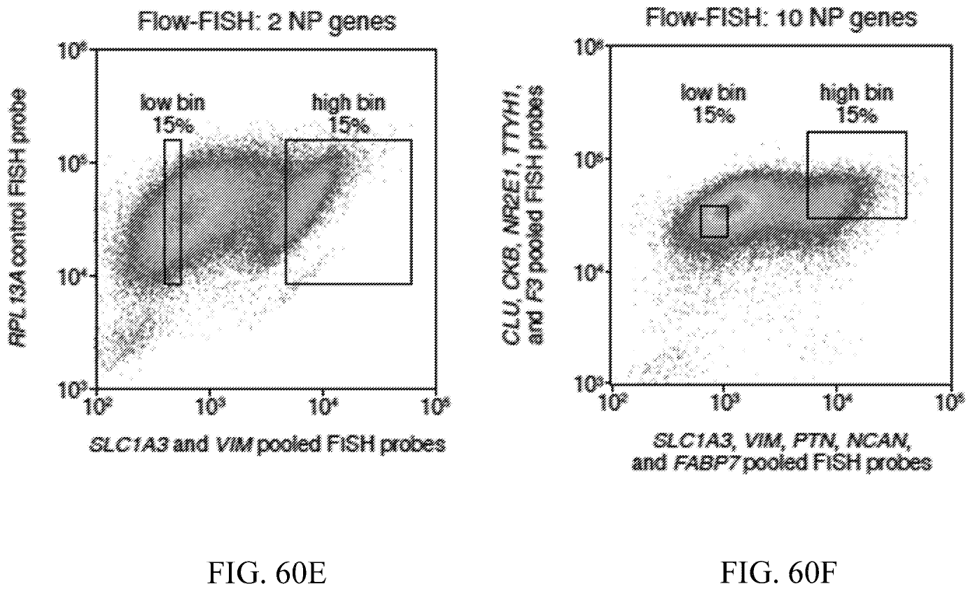

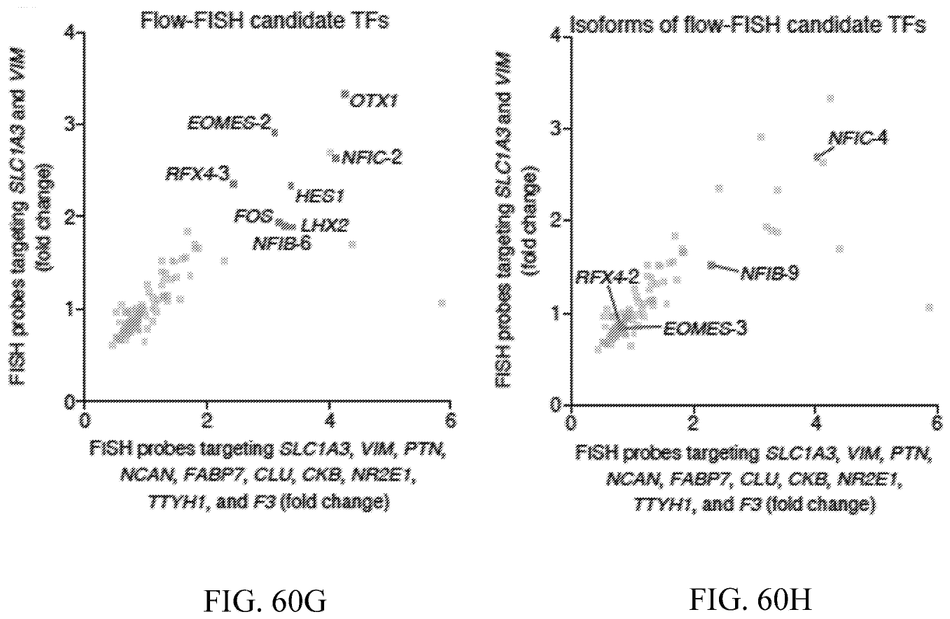

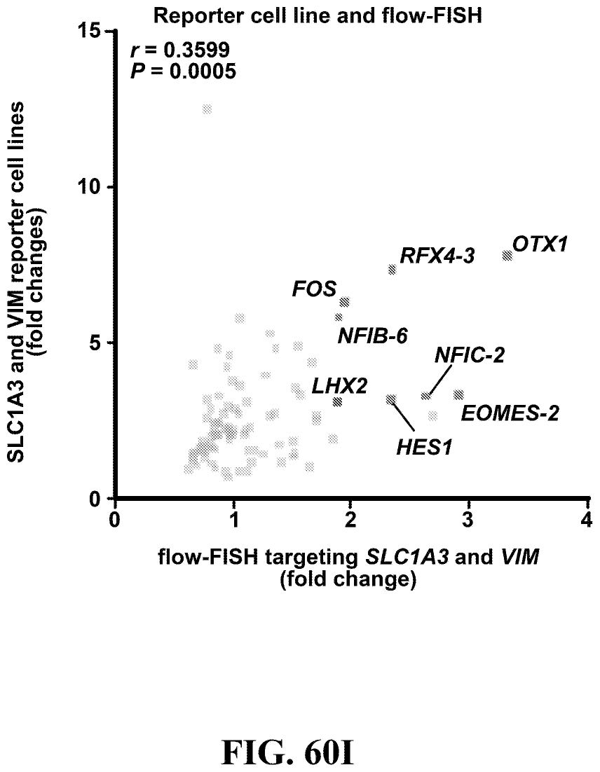

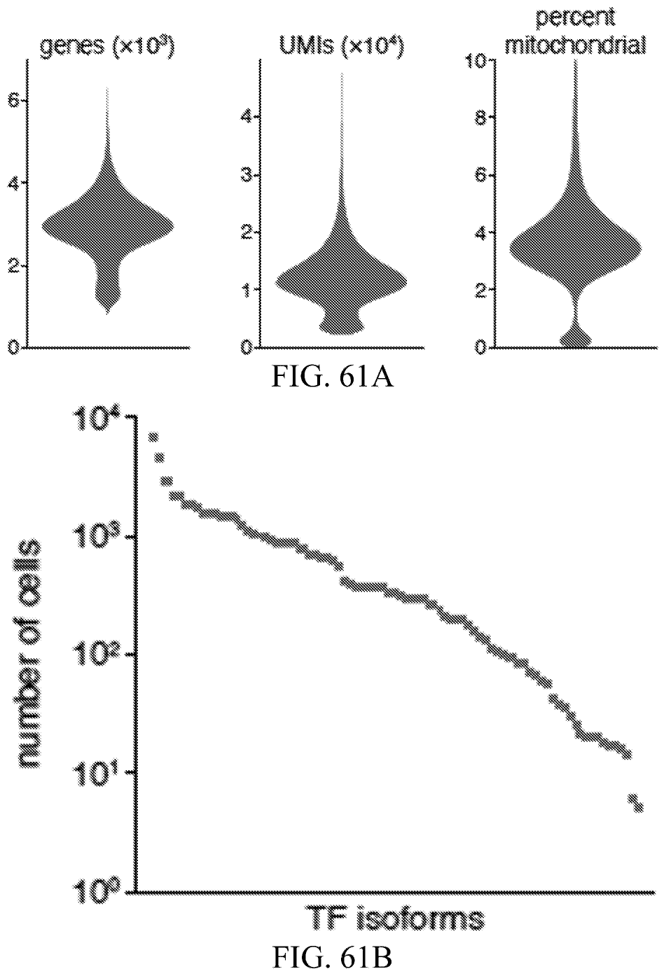

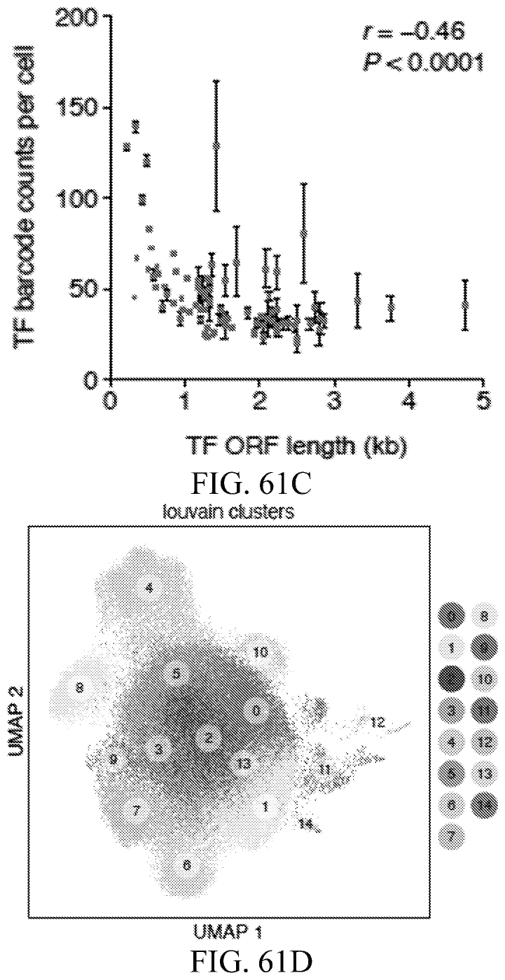

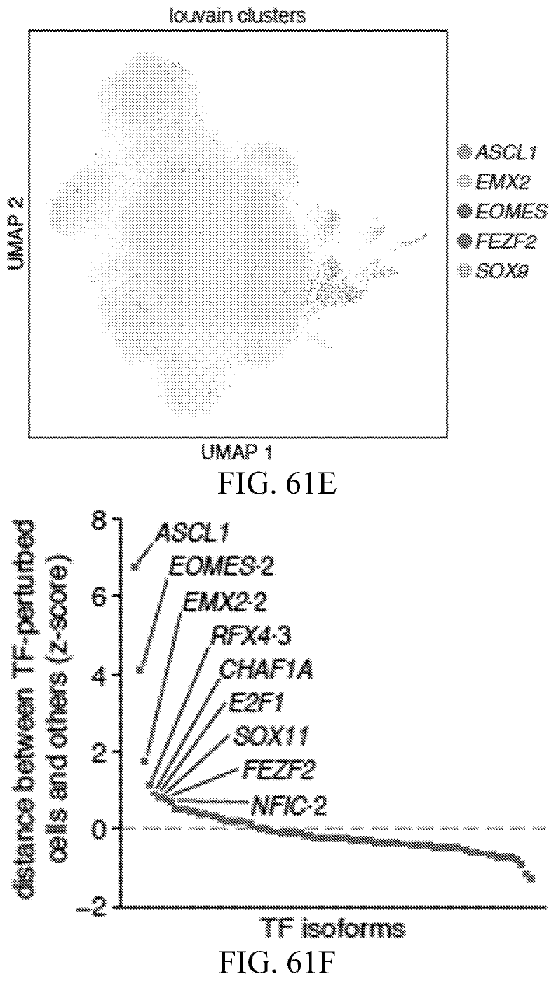

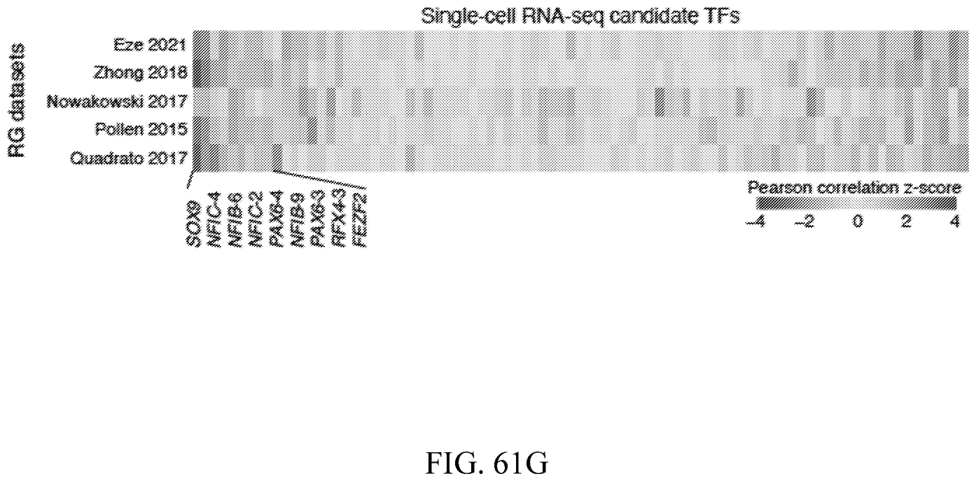

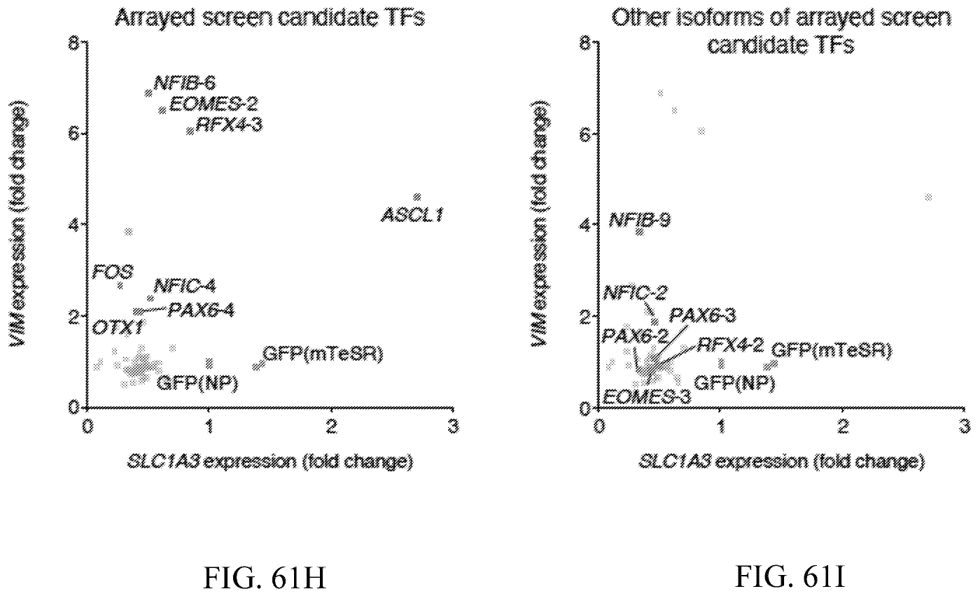

To demonstrate a generalizable approach for constructing targeted TF libraries for generation of cellular disease models, Applicants selected 90 TF isoforms specifically expressed in a selected target cell type, induced neural progenitors (iNPs), using available expression data (53-60) (Table 1; see Methods). iNPs offer a tractable model for studying neurological diseases, but current methods for producing iNPs, namely embryoid body formation (EB) (7, 18) or dual SMAD inhibition (DS) (19, 20), are low-throughput or cell line-dependent (61), respectively. Applicants introduced the pooled, targeted TF library into hESCs and differentiated the cells for 7 days (FIG. 46A and FIG. 60A). Applicants explored three different methods for selecting iNPs that can simultaneously assay different numbers of marker genes: reporter cell line (1 gene), flow-FISH (62) (2-10 genes), and scRNA-seq (up to ˜2,000 genes; FIG. 46A; see Methods). Deep sequencing of the TF barcodes identified candidate TFs that were enriched in iNPs (FIG. 46A, FIG. 60B-I, FIG. 61A-G, and Table 1). Applicants also individually tested the same 90 TF isoforms in an arrayed screening format (FIG. 42H, I). Applicants obtained concordant screening results (maximum Spearman correlation P-value of 10-3) with overlapping sets of top candidate TFs for iNP differentiation (FIG. 46B and Table 1), some of which (NFIB (63), OTX1 (64), PAX6 (65, 66), EOMES (66, 67), and ASCL1 (68)) are known to be critical for neural development, further supporting the screening results.

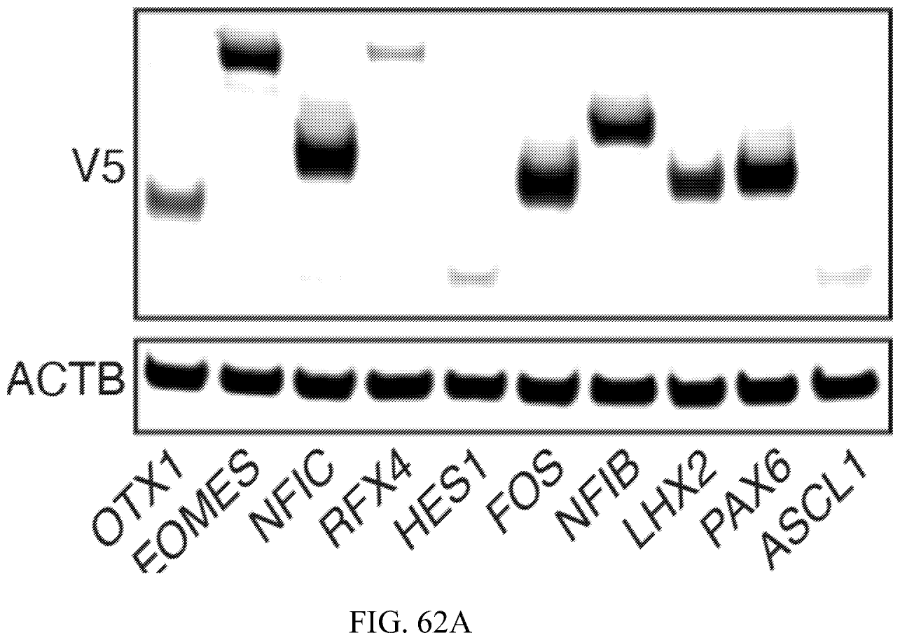

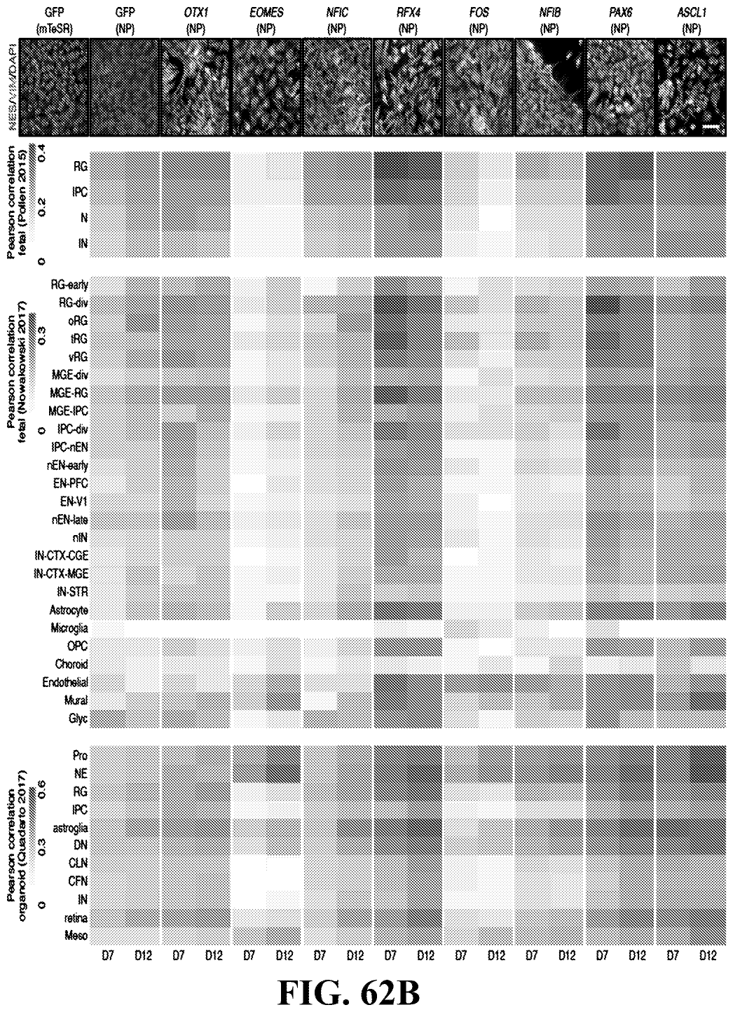

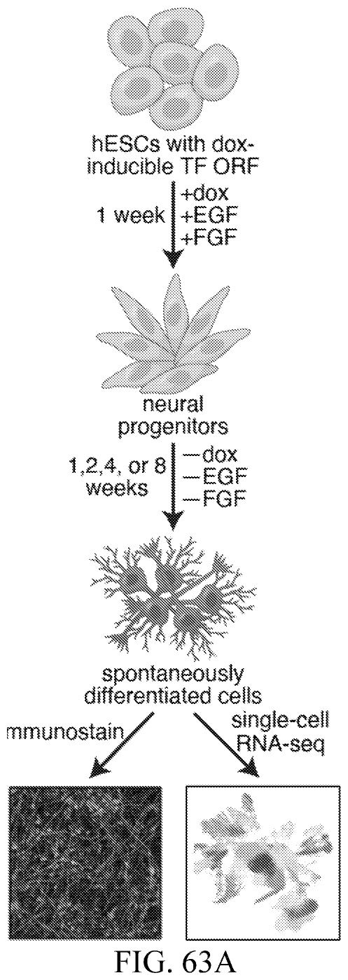

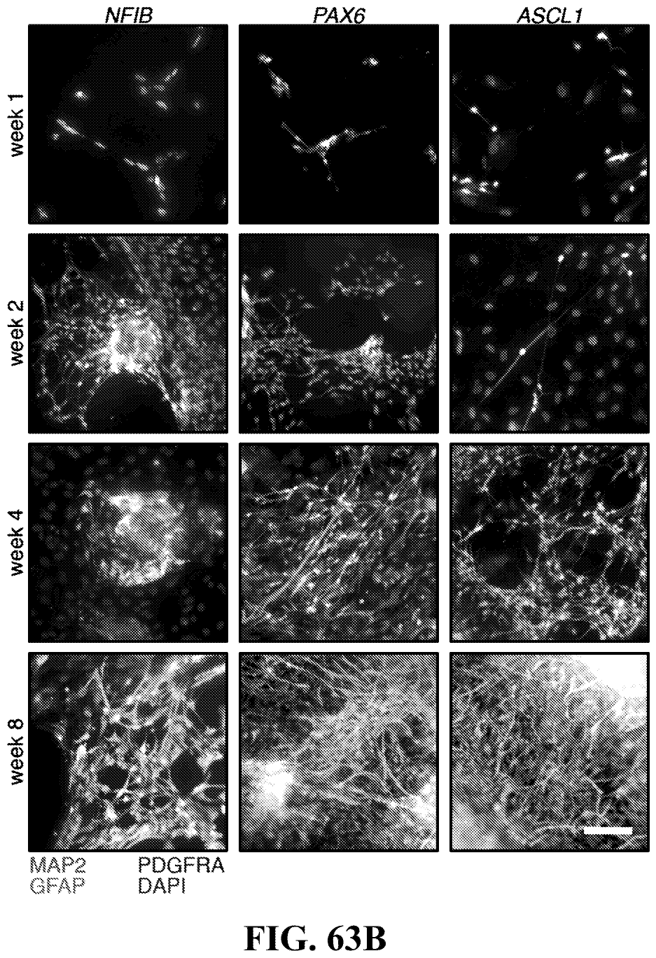

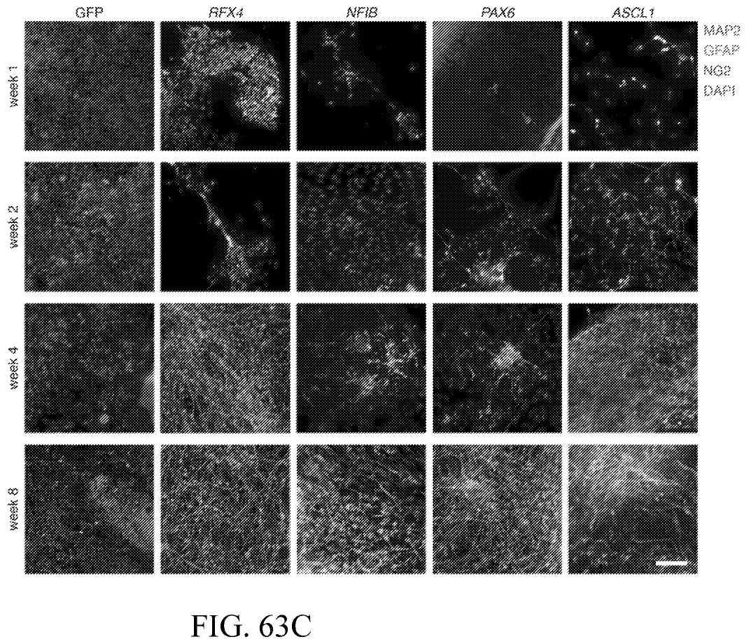

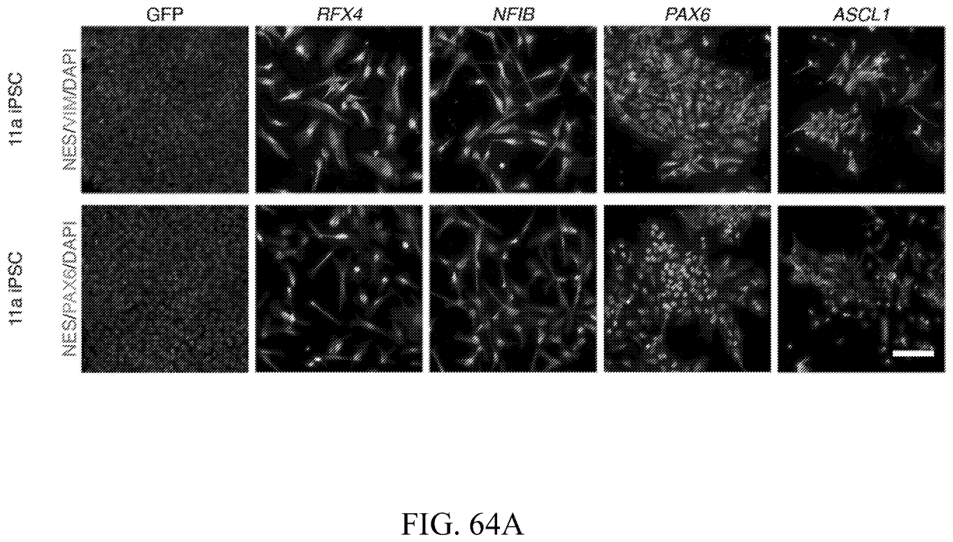

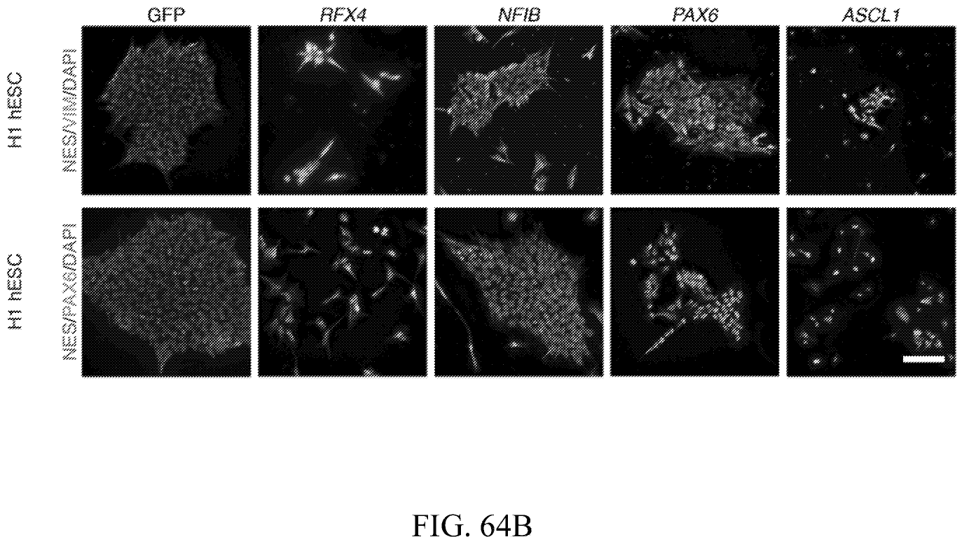

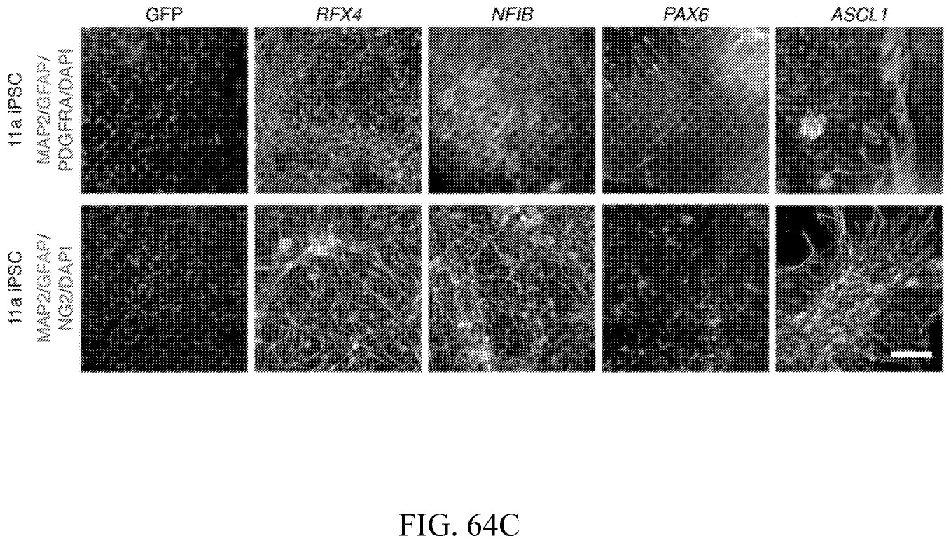

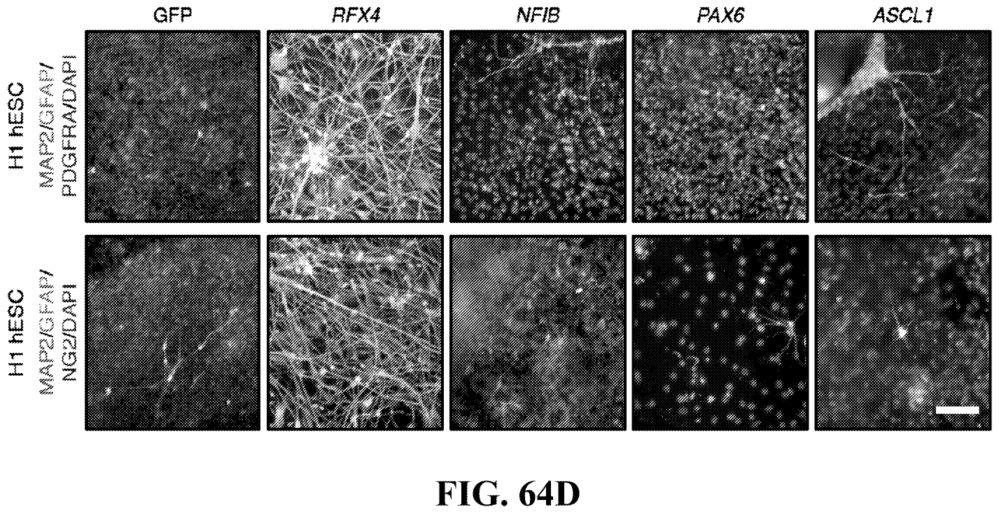

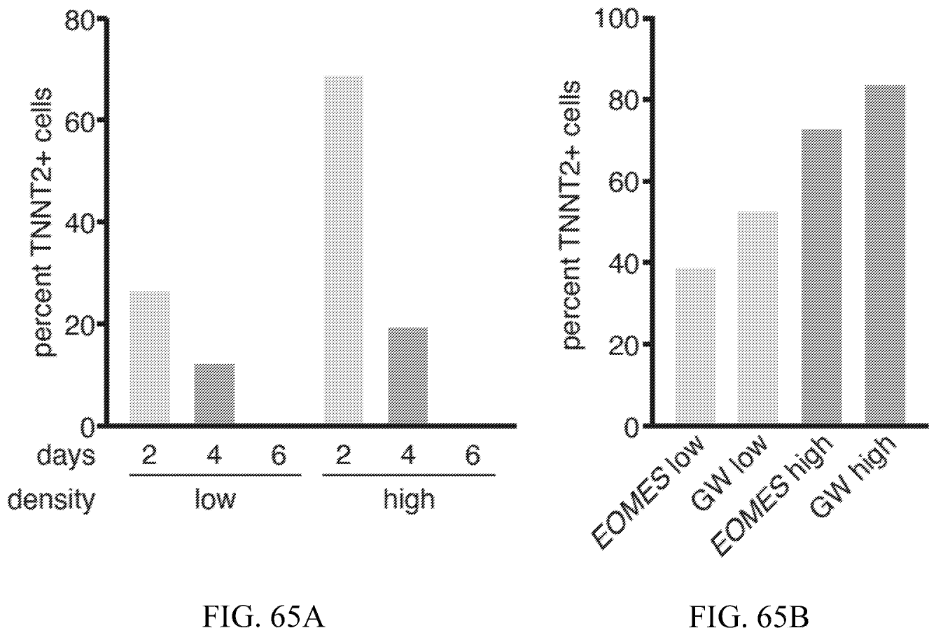

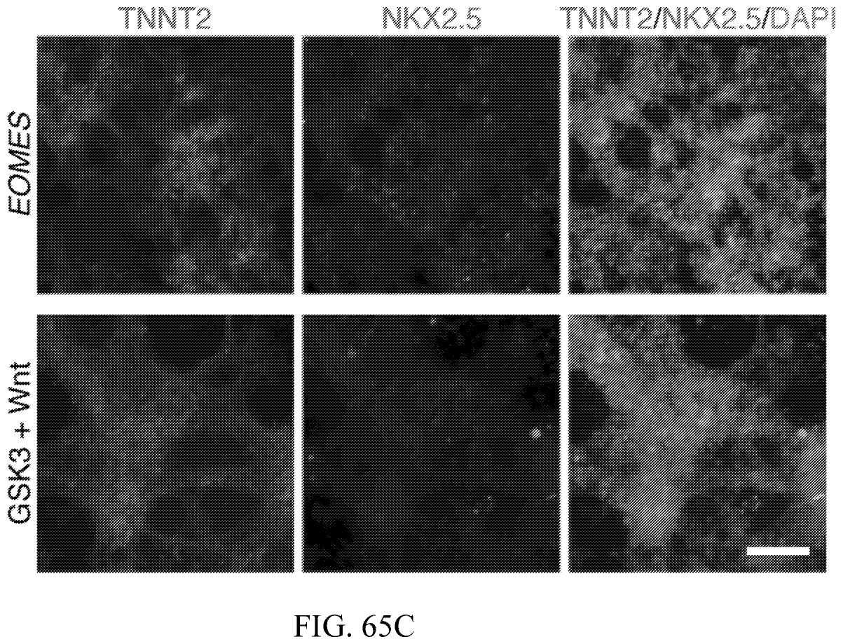

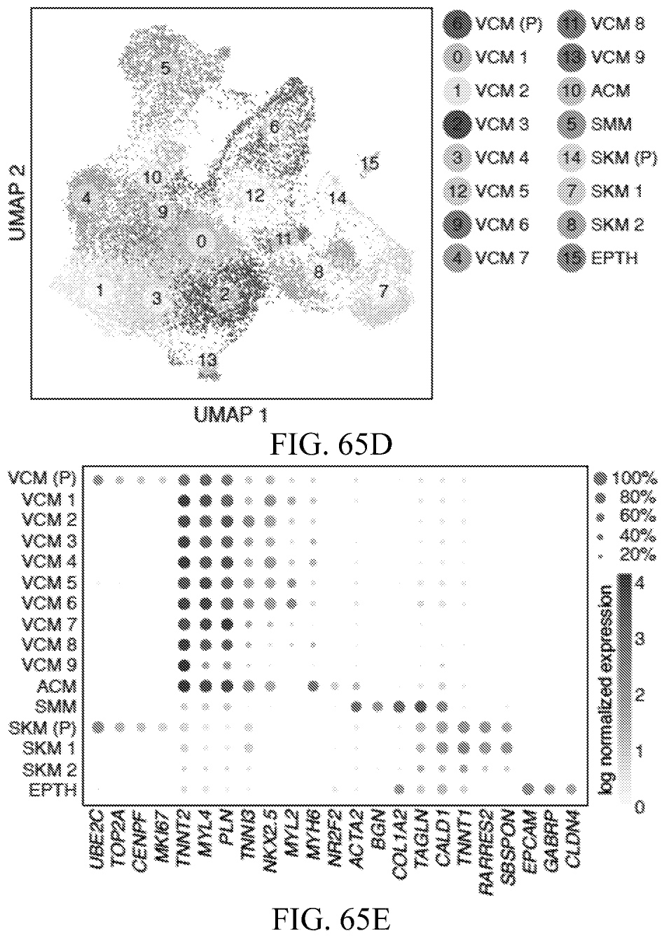

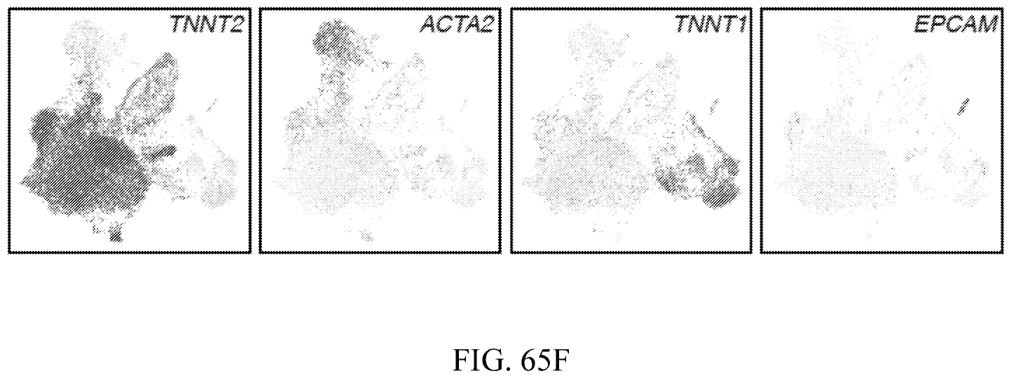

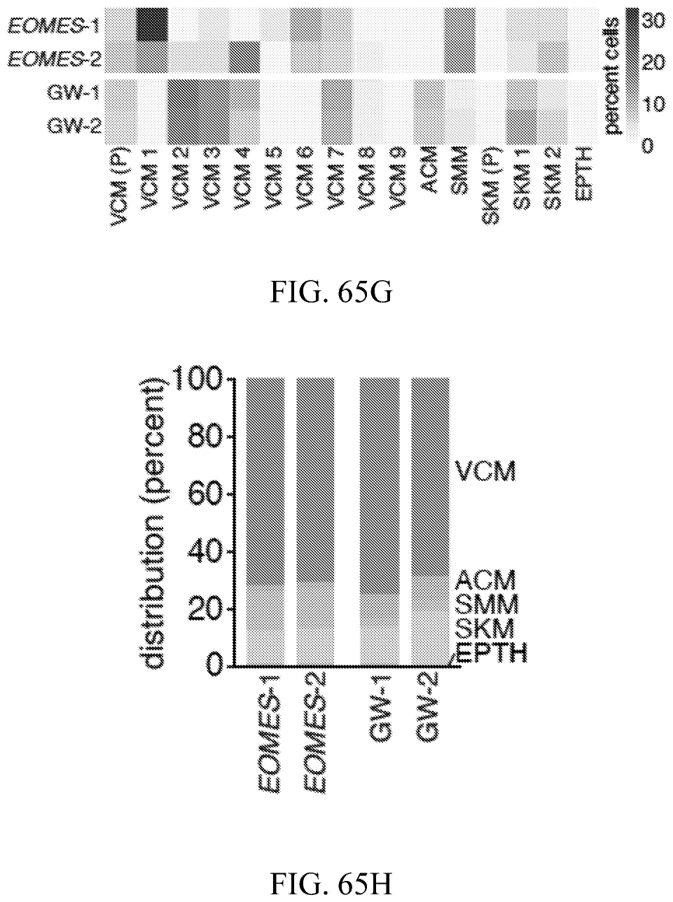

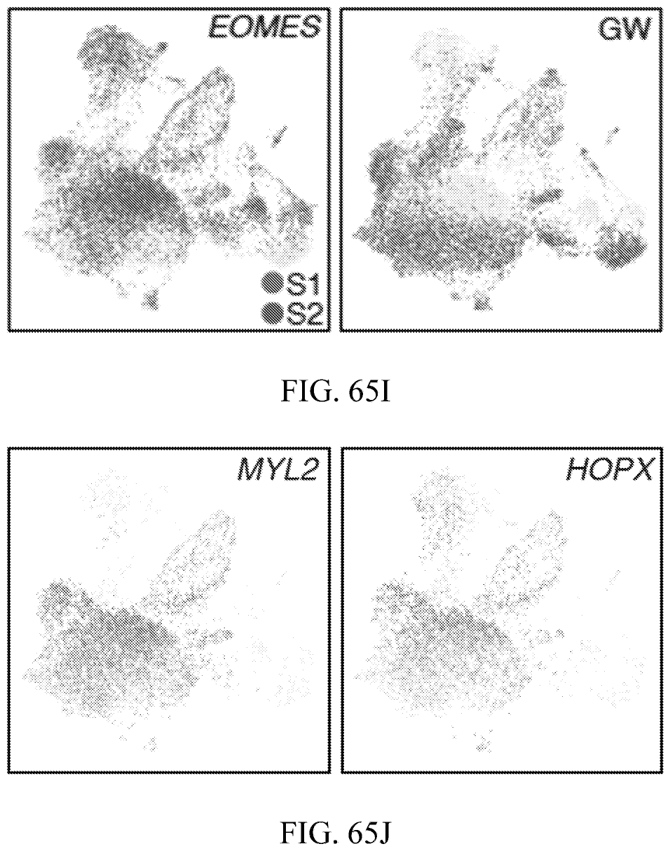

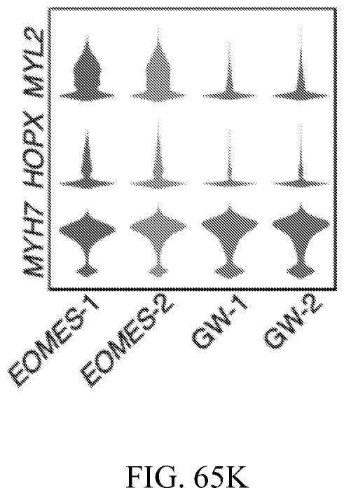

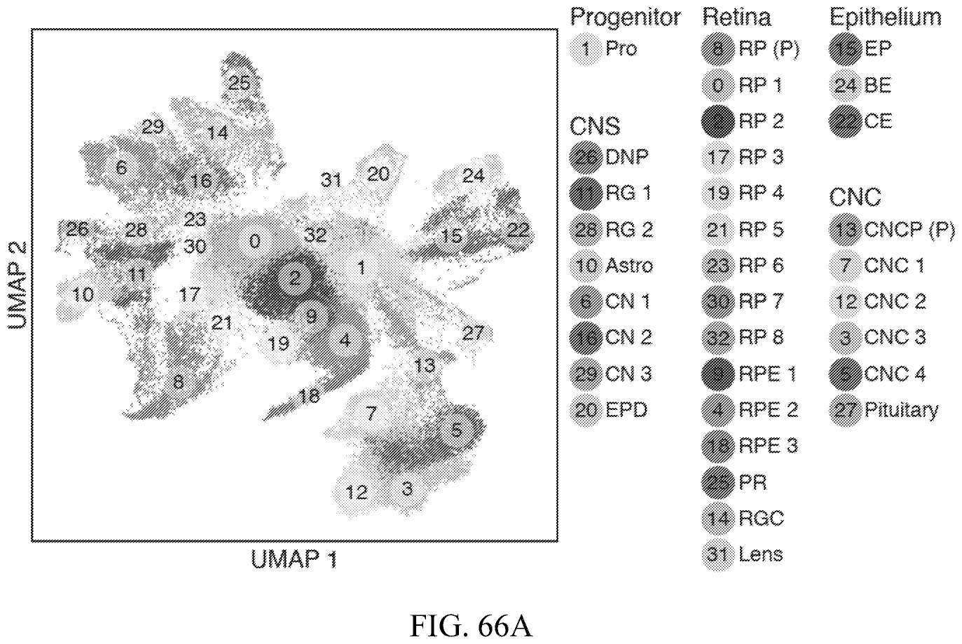

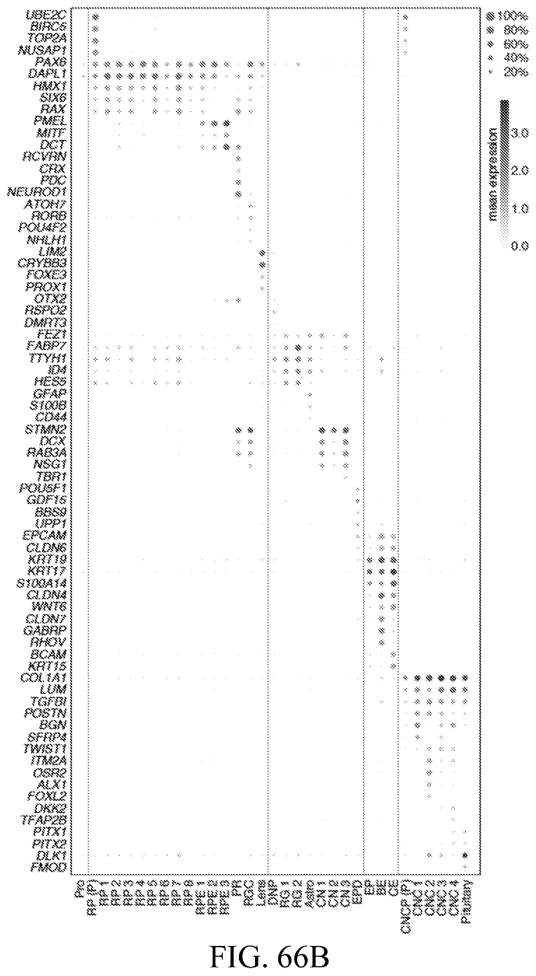

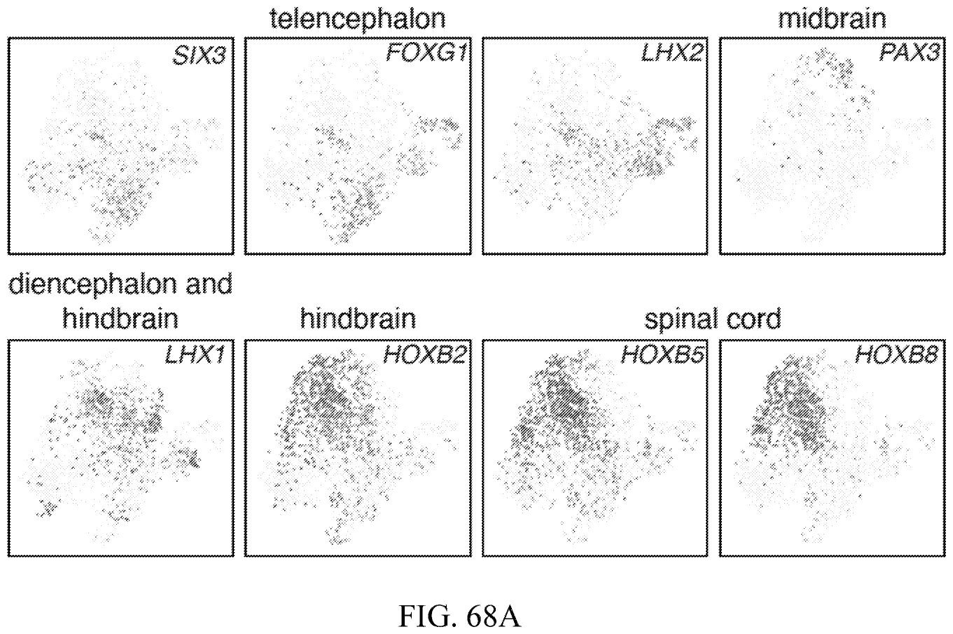

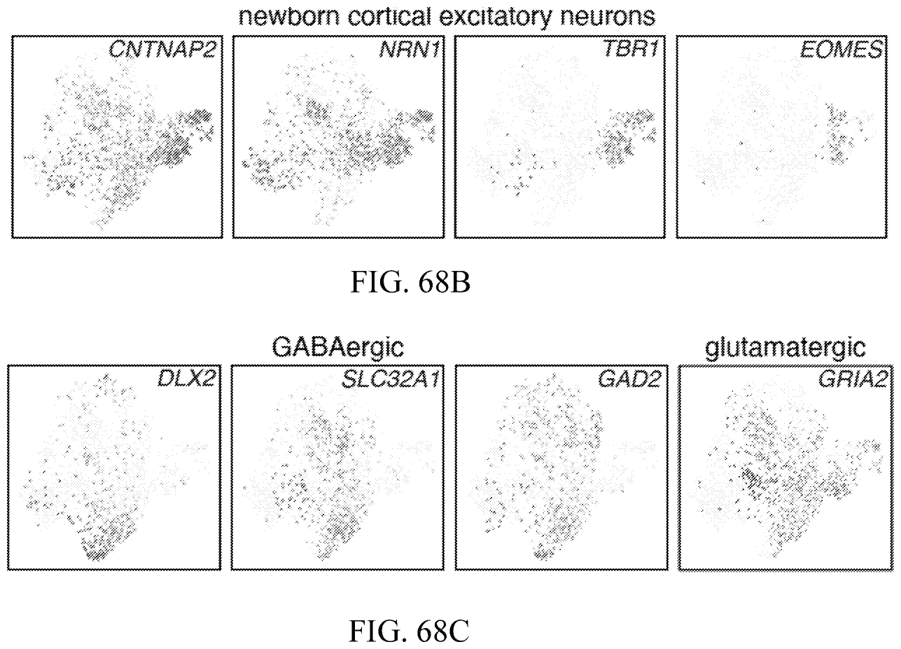

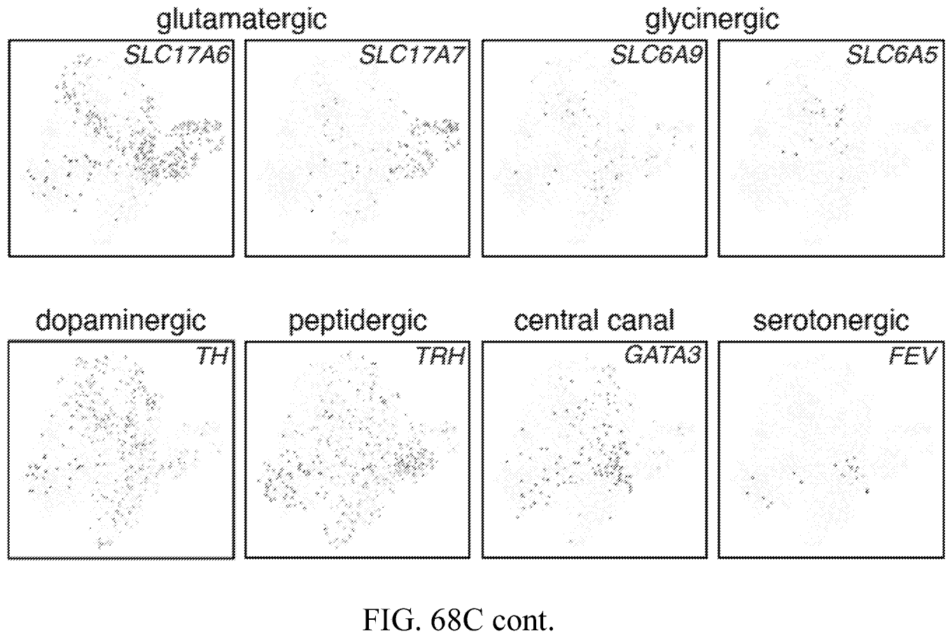

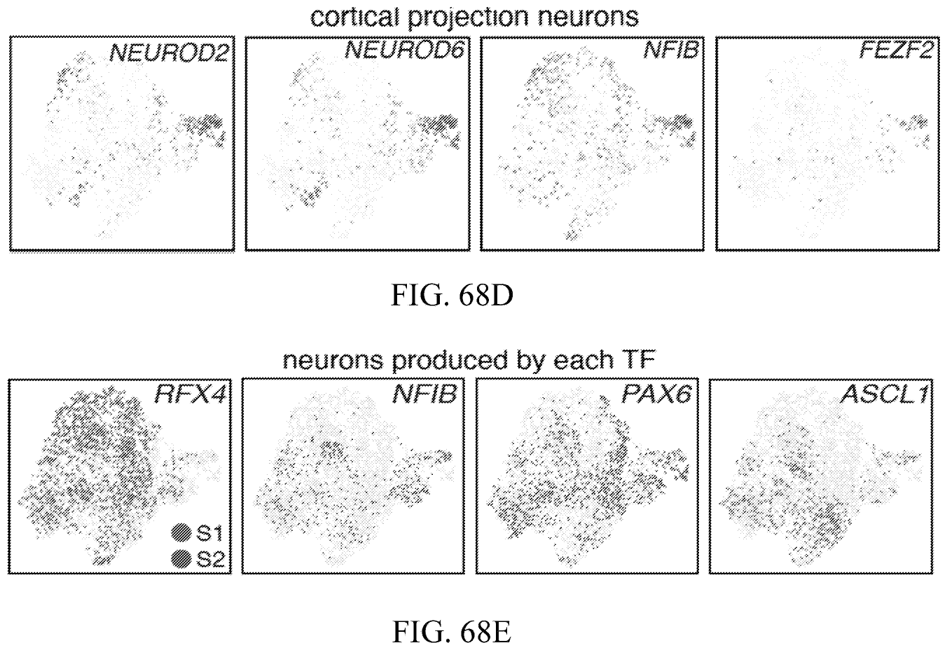

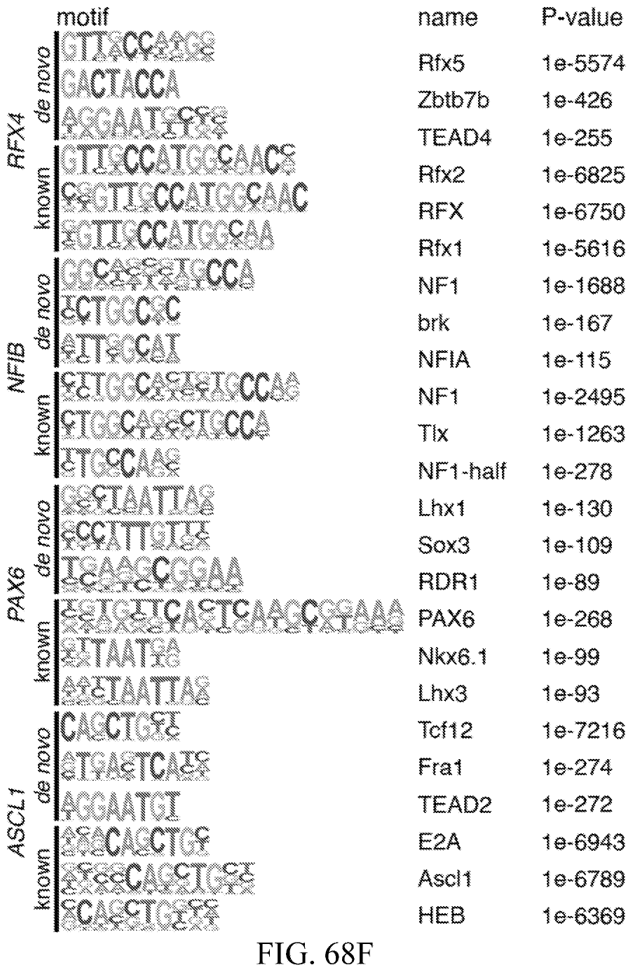

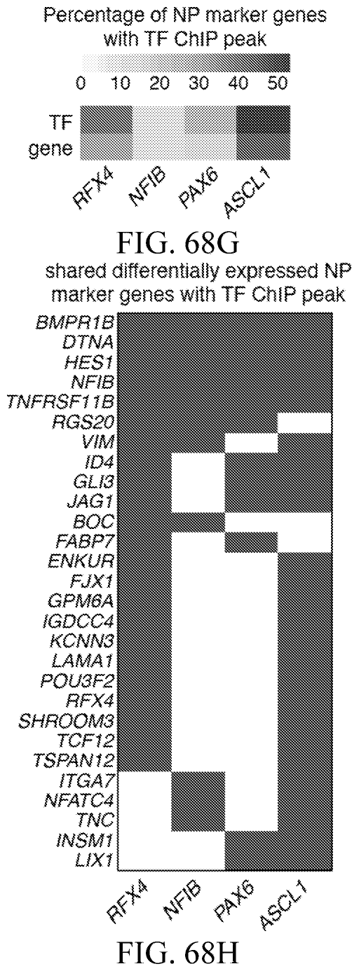

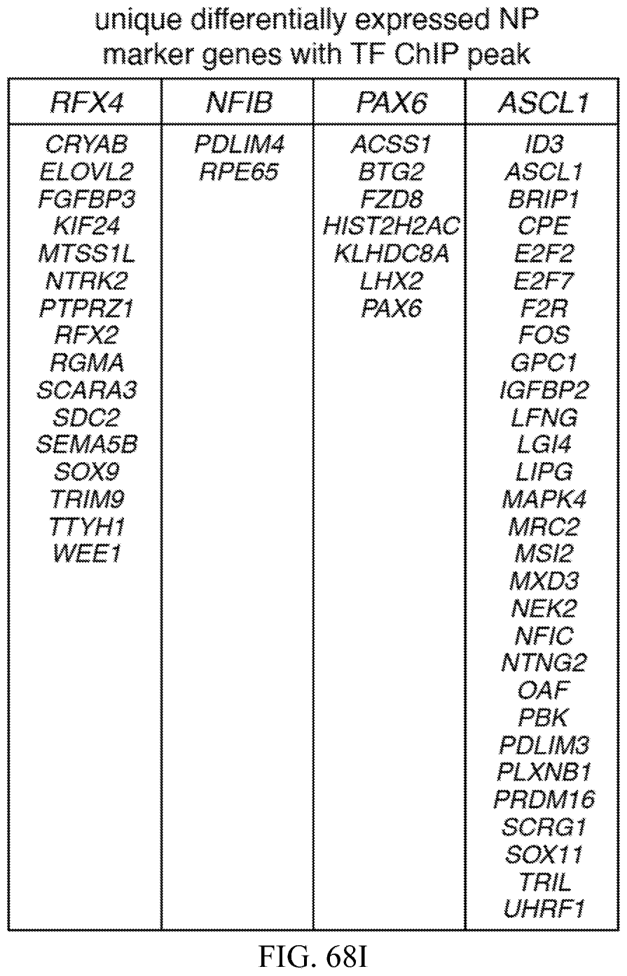

For downstream analysis, Applicants focused on eight TFs (RFX4, NFIB, FOS, OTX1, NFIC, PAX6, EOMES, and ASCL1) that were enriched in at least two screens. While all eight TFs produced iNPs that expressed VIM, an NP marker used to select target cells in the screens, RFX4, ASCL1, and PAX6 produced iNPs with bulk RNA-seq expression signatures that were the most similar to human fetal and organoid NPs (FIG. 62) (56, 69, 70). Overexpression of four of the eight TFs (RFX4, NFIB, PAX6, and ASCL1) produced multipotent iNPs that, like NPs, could spontaneously differentiate into neurons and astrocytes, as assayed by immunostaining and scRNA-seq (FIG. 46C, FIG. 63-64, Note 1). Intriguingly, cells overexpressing EOMES spontaneously differentiated into cardiomyocytes by both immunostaining and scRNA-Seq (FIG. 65 and Note 2). ScRNA-seq profiles of cells spontaneously differentiated from iNPs expressing these four TFs revealed a broad range of cell types that was reproducible across replicates and distinct between TFs, with RFX4-iNPs producing more CNS cell types (FIG. 66-68, Note 3, Table 6). Chromatin immunoprecipitation with sequencing (ChIP-seq) targeting each of the four TFs allowed us to identify motifs, transcriptional co-regulators, and candidate genes that drive iNP differentiation (FIG. 68F-I, Note 4).

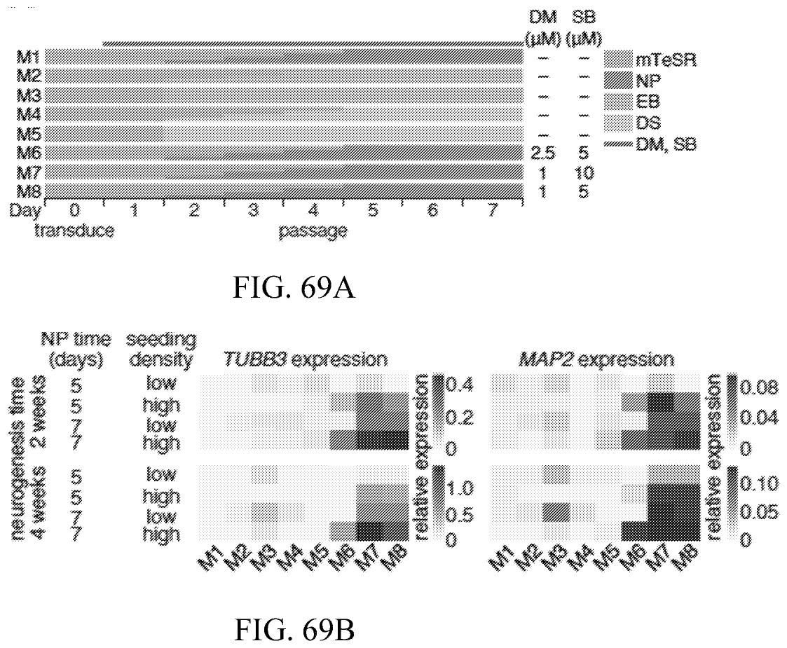

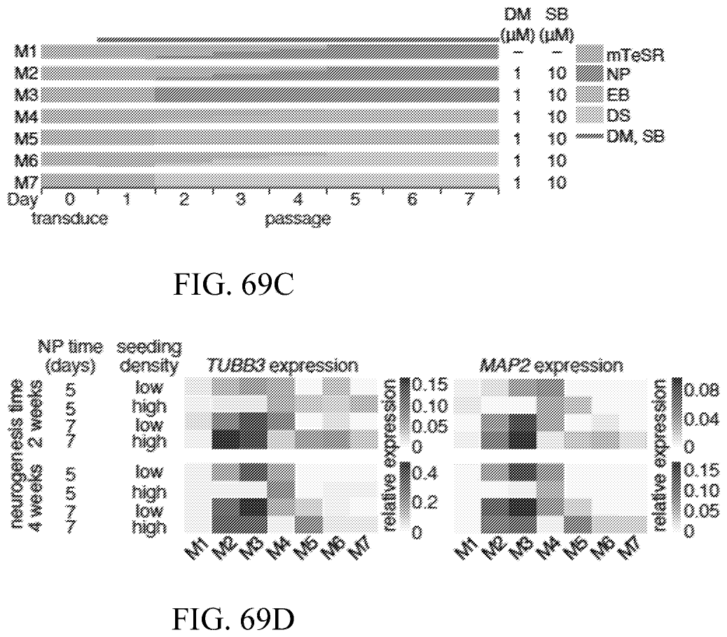

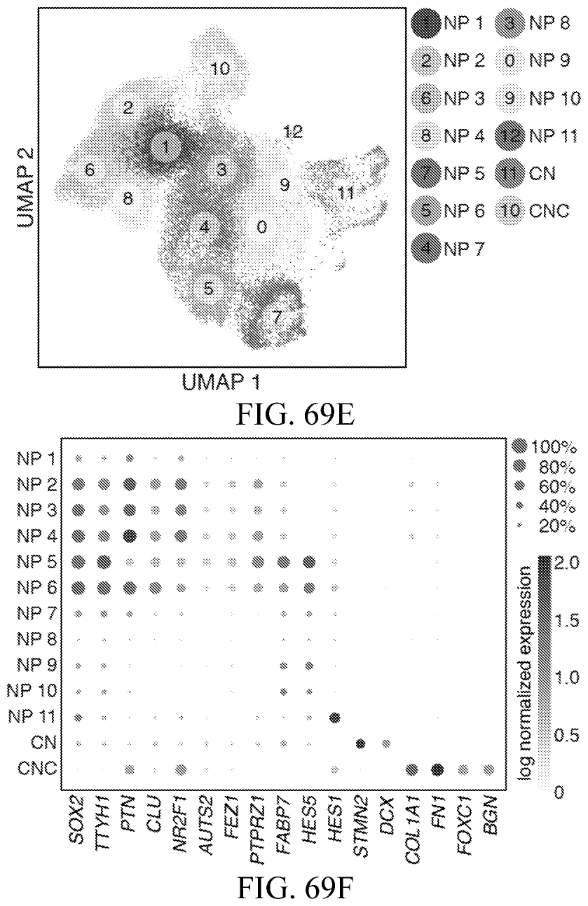

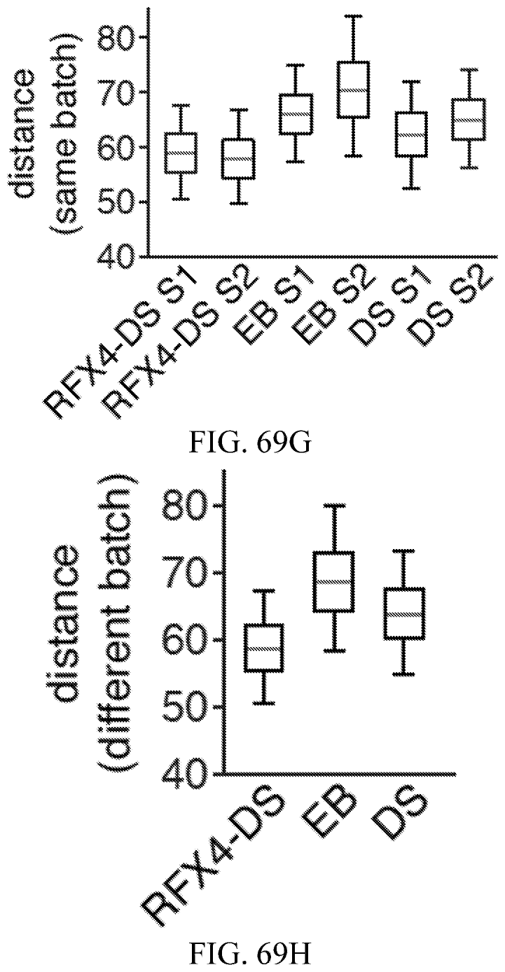

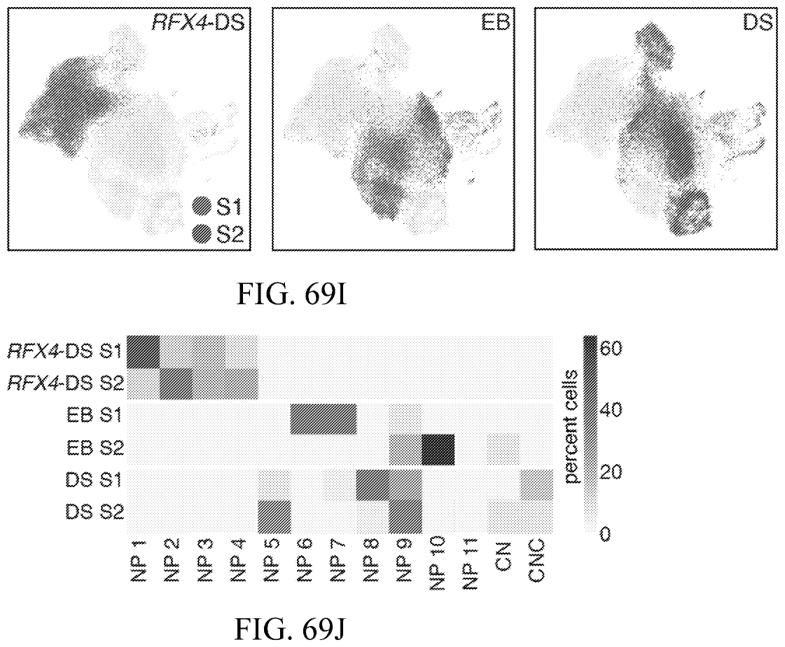

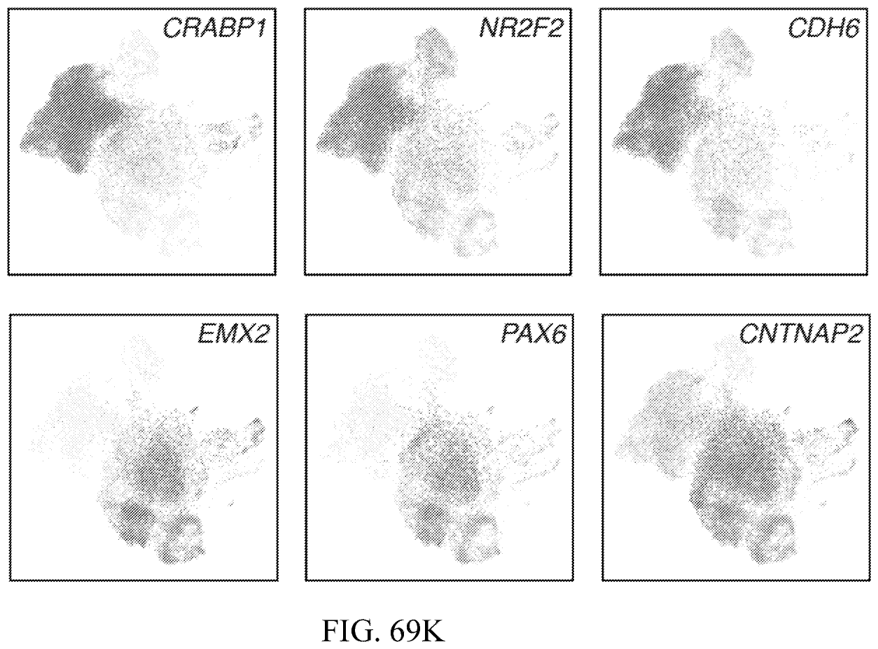

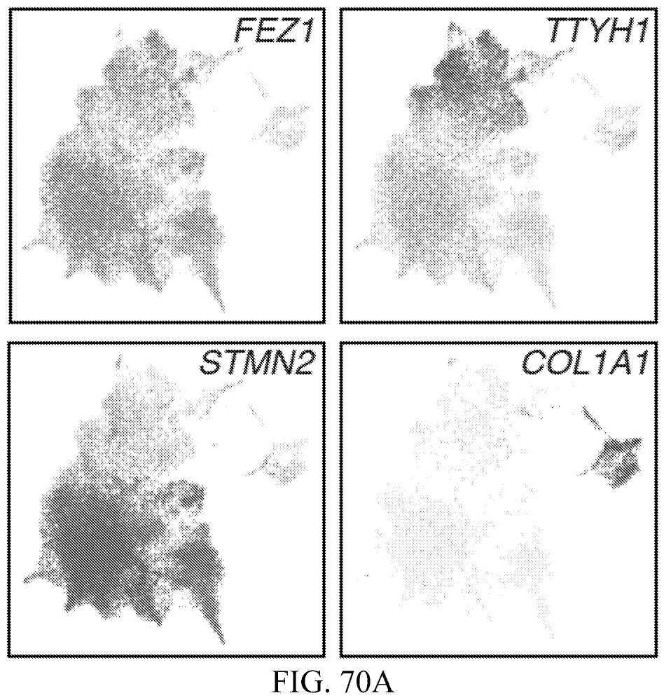

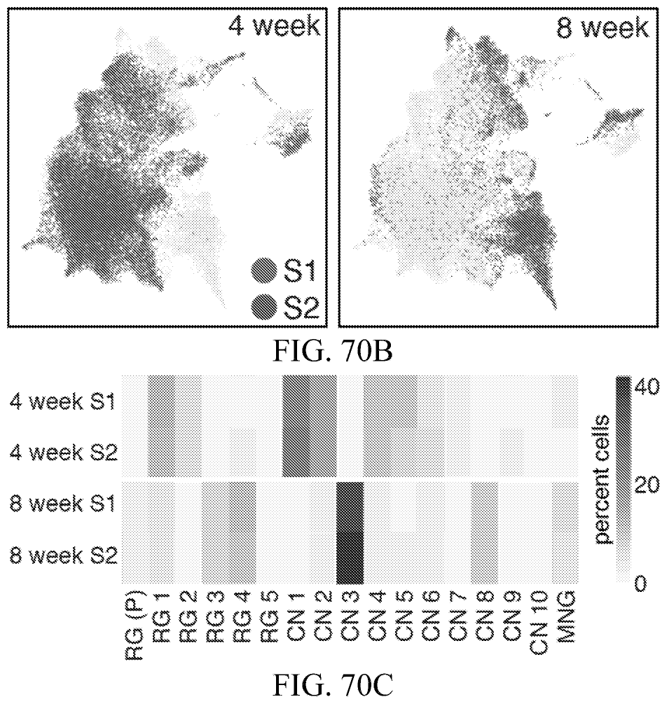

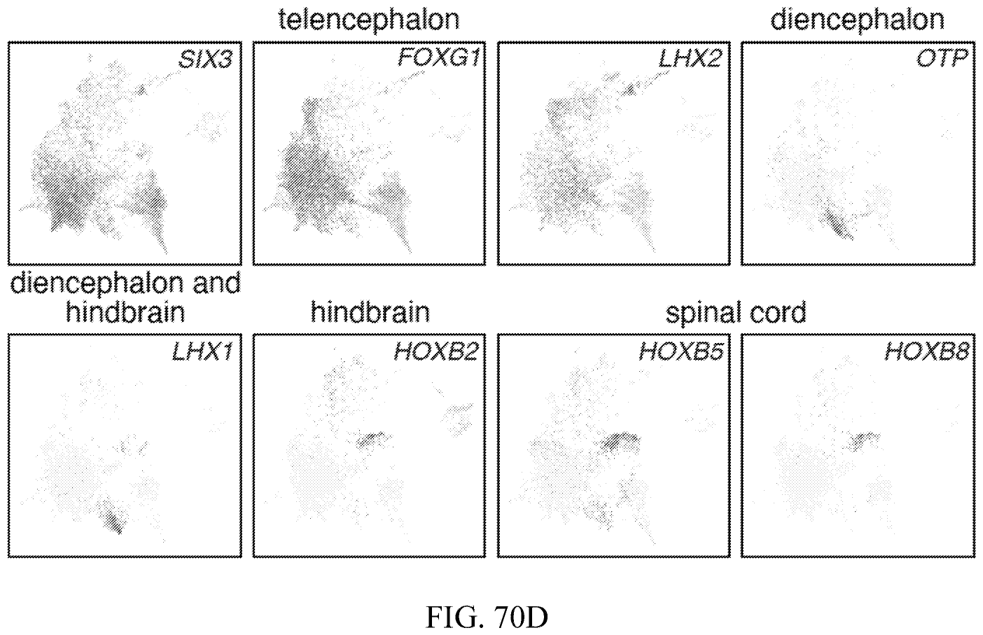

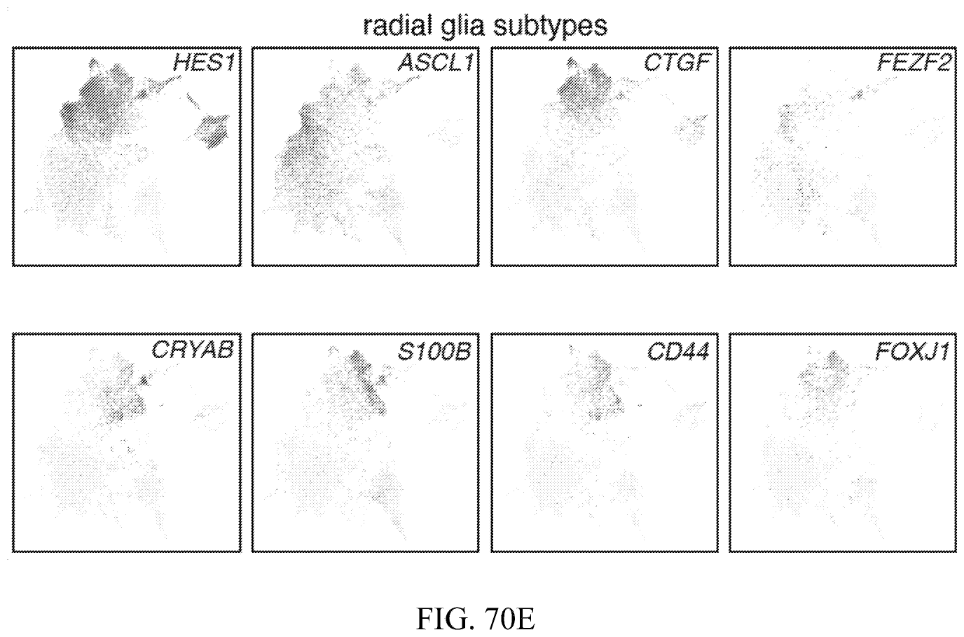

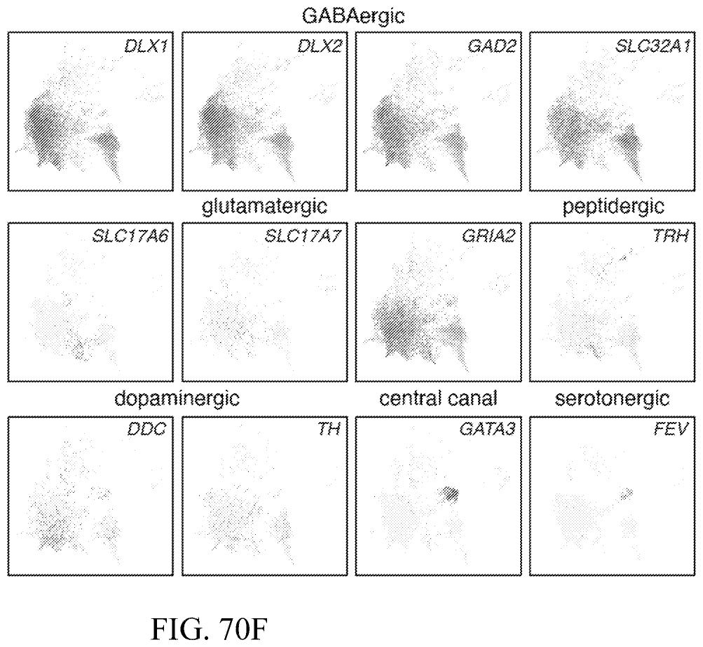

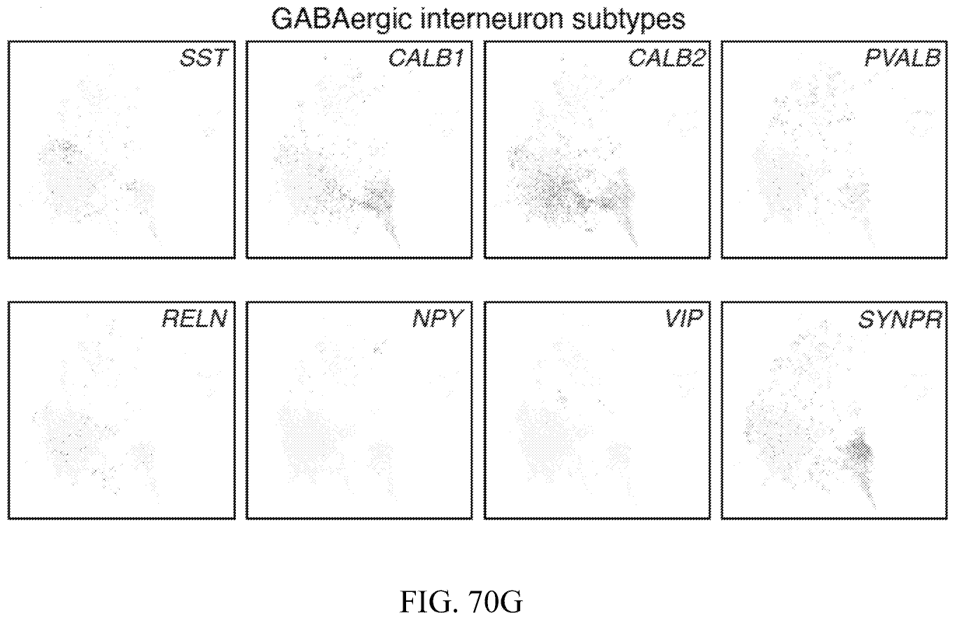

RFX4-iNPs as a reproducible and tractable model for neural development and disease. Applicants further optimized RFX4-iNPs by combining RFX4 overexpression with dual SMAD inhibition (FIG. 69A-D) and compared Applicants' optimized protocol (RFX4-DS) to two previous NP differentiation methods (7, 33) using scRNA-seq. RFX4-DS-iNPs were most consistent within and between replicates, as measured by pairwise Euclidean distances and cluster distributions (FIG. 69E-K and Table 6), and may resemble subpallial NPs (71). Moreover, spontaneously differentiated cells from RFX4-DS-iNPs were remarkably consistent between replicates and consisted of predominantly radial glia and neurons, with a small subset (2-6%) of meningeal cells (FIG. 46D-F, FIG. 70, and Table 6). The propensity for RFX4-DS-iNPs to spontaneously differentiate into GABAergic neurons (FIG. 70F, G), rather than glutamatergic neurons like iNPs produced by alternative methods (7, 20), may stem from higher levels of NR2F2, a marker gene for GABAergic interneurons (FIG. 69K) (72, 73). Integrated analysis of ChIP-seq and bulk RNA-seq data further suggests that RFX4 directly binds the NR2F2 locus and upregulates its expression.

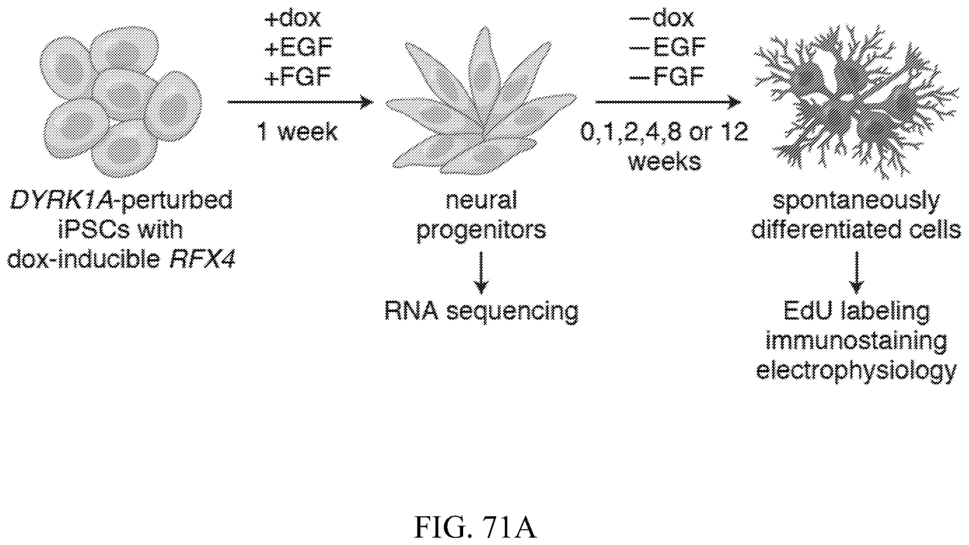

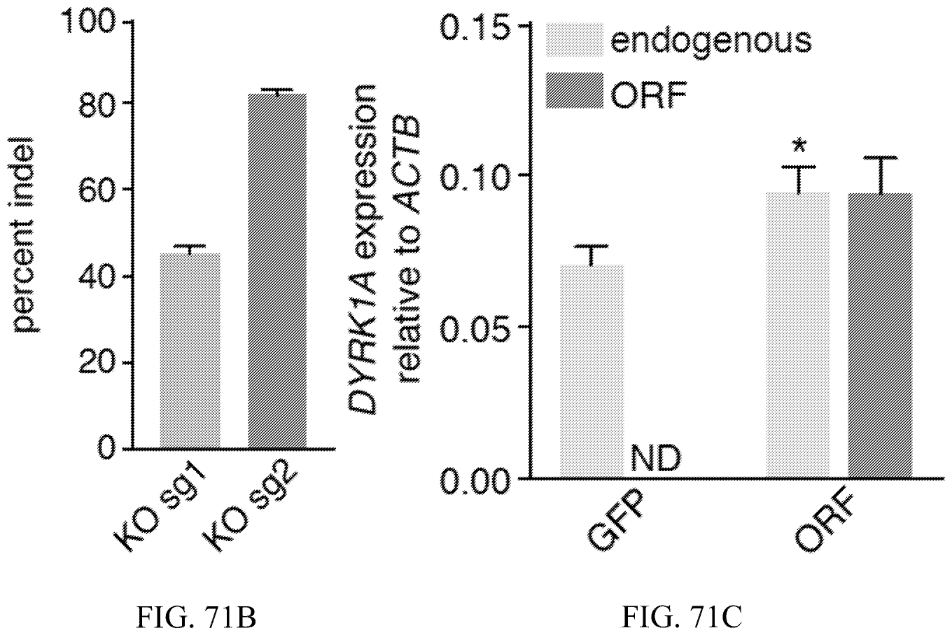

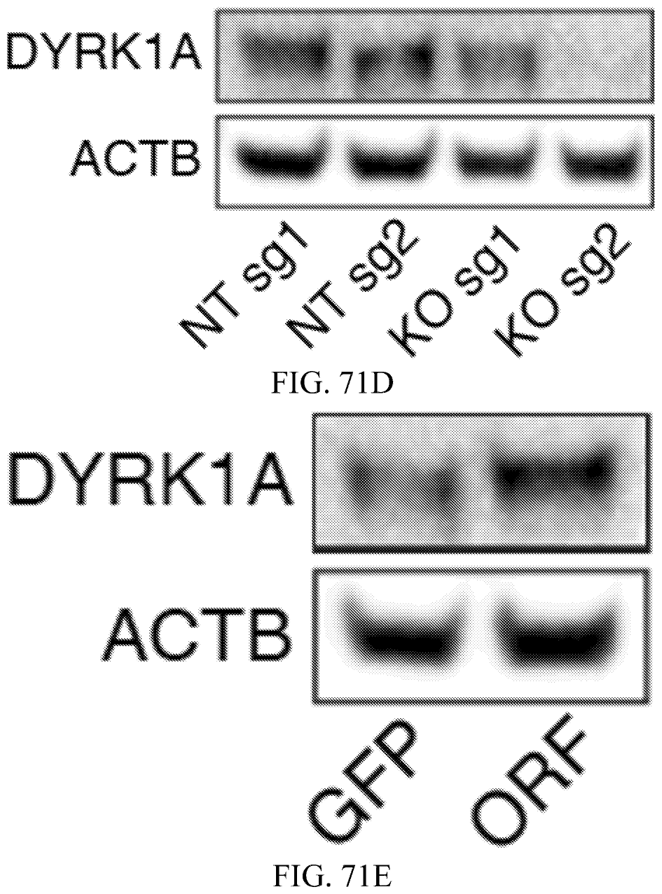

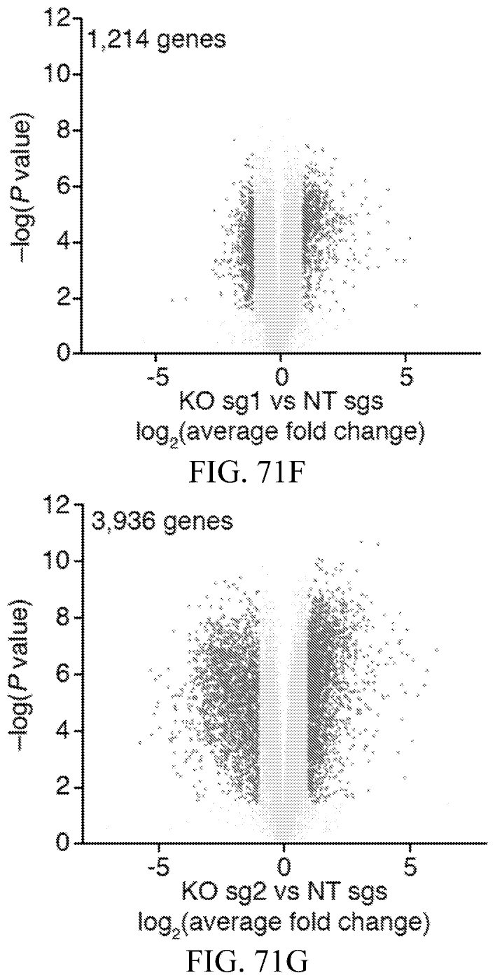

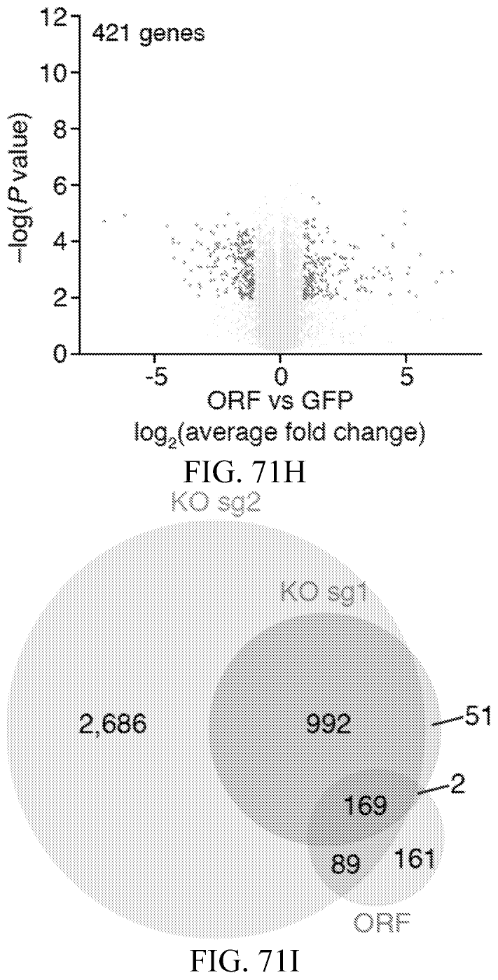

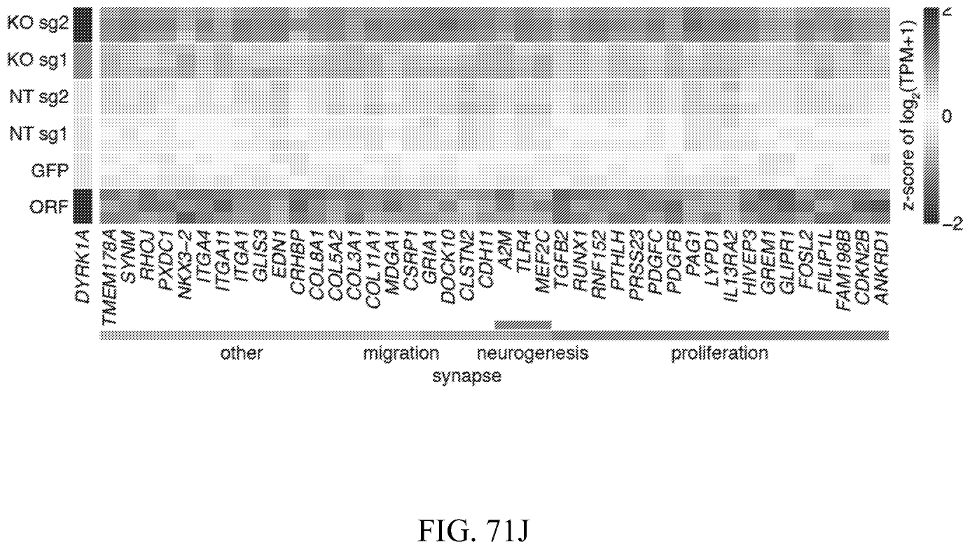

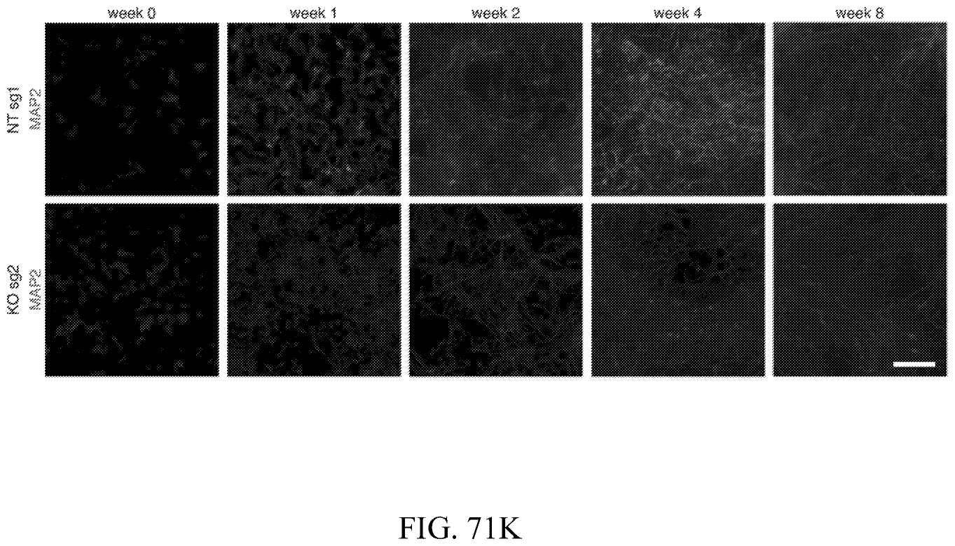

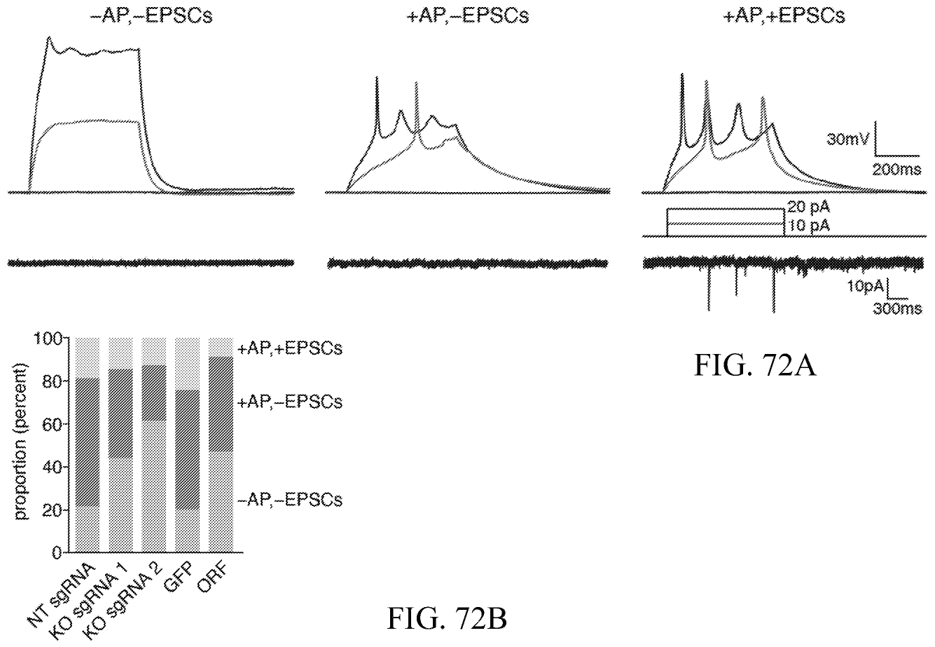

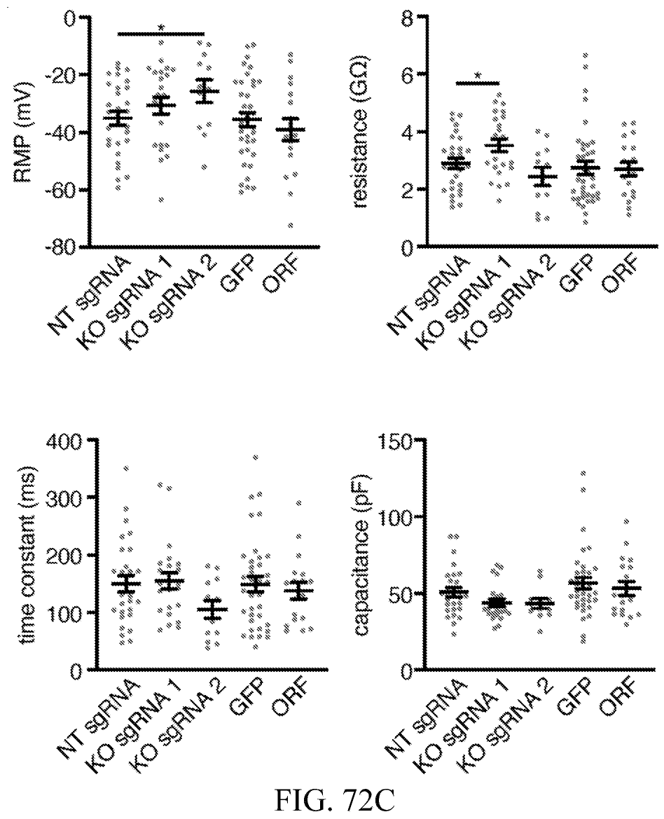

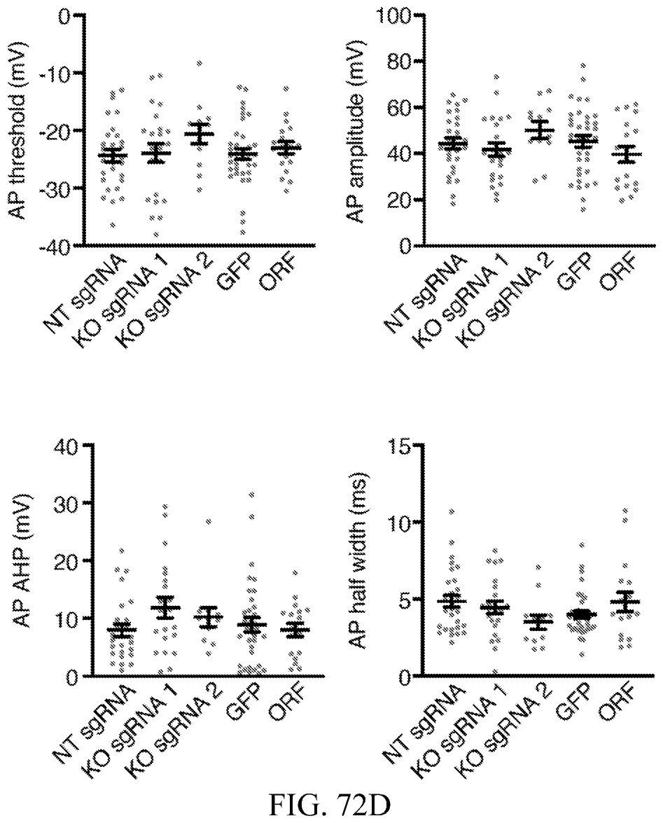

To explore the utility of RFX4-iNPs for modeling neurological disorders, Applicants evaluated the effects of DYRK1A perturbation on neurogenesis in this model (FIG. 71A-E). DYRK1A knockout and overexpression have been implicated in autism spectrum disorder (74, 75) and Down syndrome (76), respectively. Bulk RNA-seq of DYRK1A knockout and overexpression identified 42 genes, including those involved in neuronal migration and synapse formation, that were expressed in a DYRK1A dosage-dependent manner (FIG. 71F-J). During spontaneous differentiation, DYRK1A knockout increased, whereas DYRK1A overexpression decreased, the proportion of proliferating iNPs (FIG. 46G, H). Interestingly, DYRK1A knockout iNPs and overpressing iNPs ultimately have reduced neurogenesis as measured by neuronal MAP2 staining: in the knockout, this is because increased iNP proliferation deters neurogenesis (FIG. 461 and FIG. 71K), whereas in DYRK1A overexpression iNPs, it is because there were fewer iNPs due to lower initial proliferation (FIG. 46J). Electrophysiological characterization of spontaneously differentiated neurons showed that both DYRK1A knockout and overexpression resulted in reduced proportions of neurons with properties indicative of maturation (FIG. 72). Applicants' results are consistent with previous DYRK1A studies in other model systems (77-83) and provide additional insight. Thus, RFX4-iNPs may serve as a tractable system for studying neural development and disease.

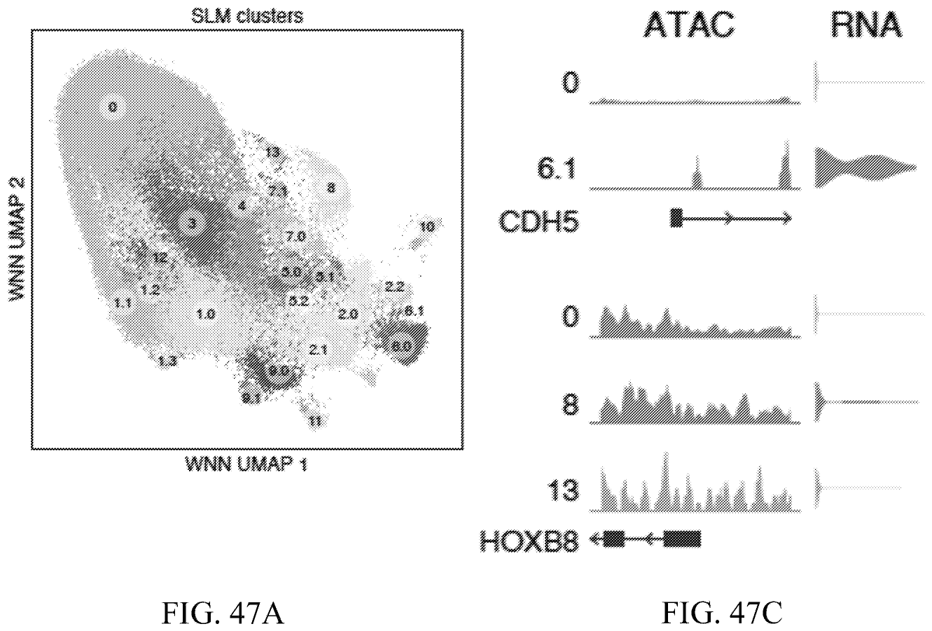

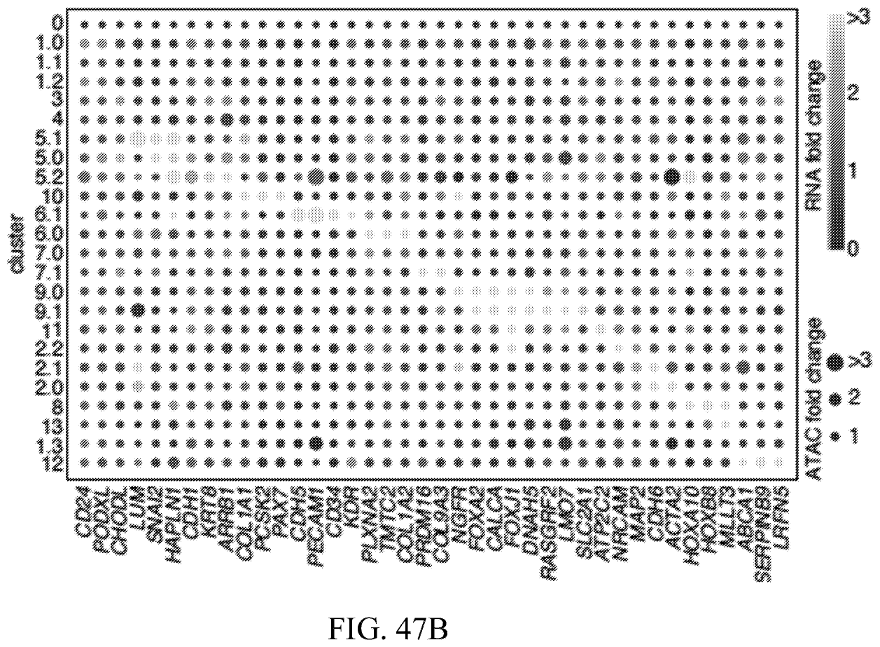

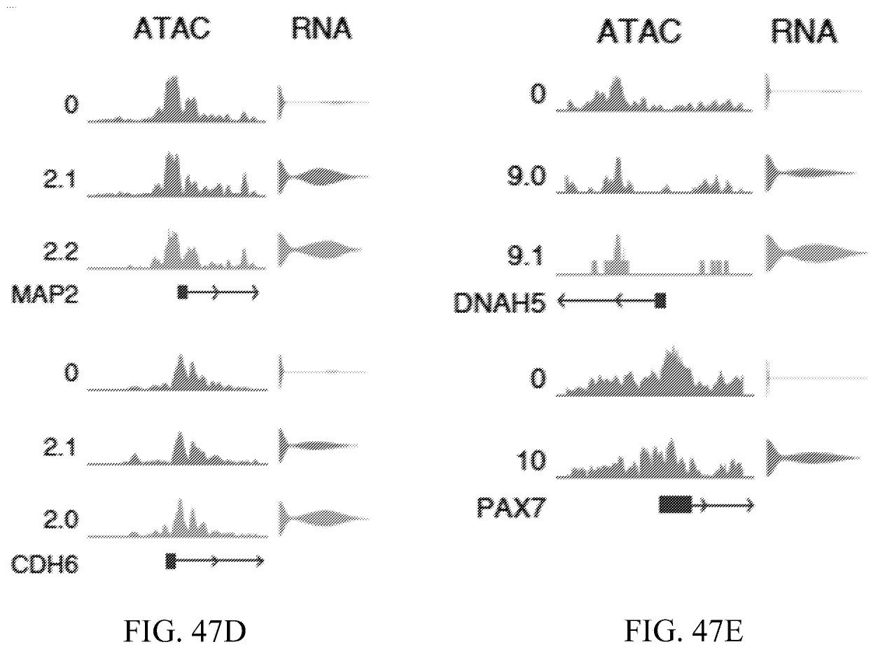

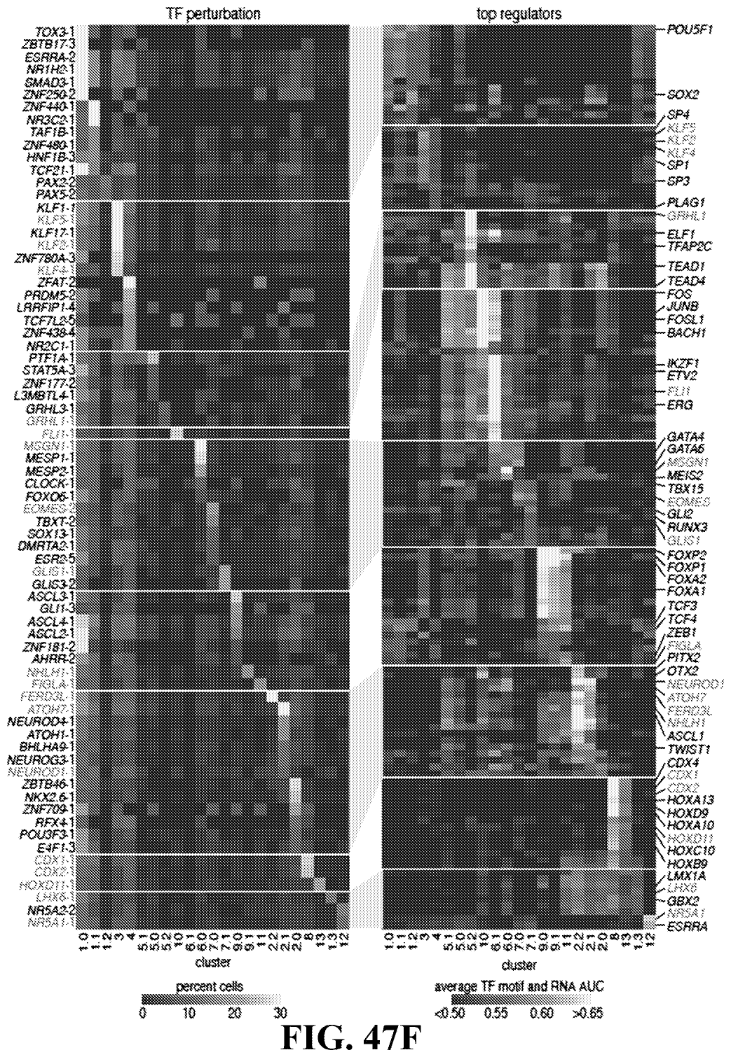

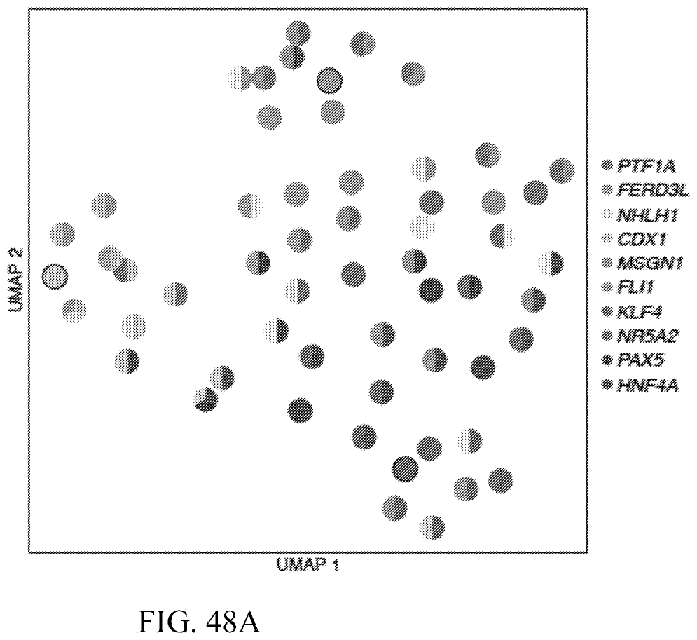

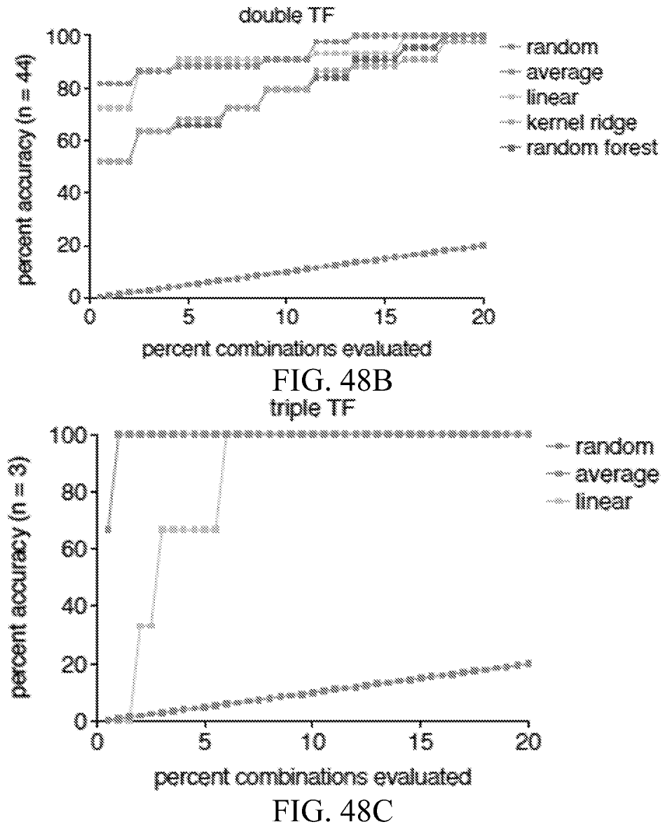

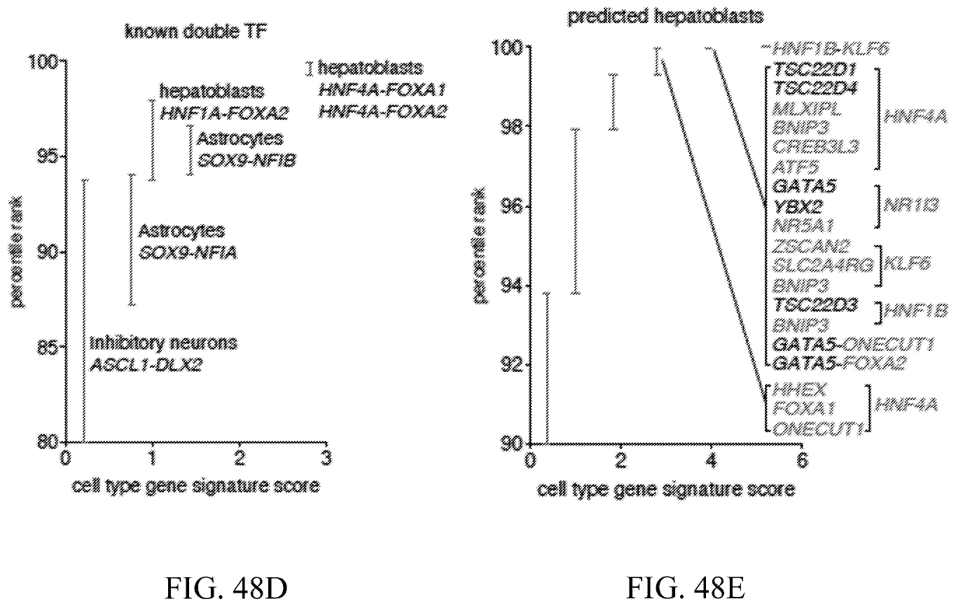

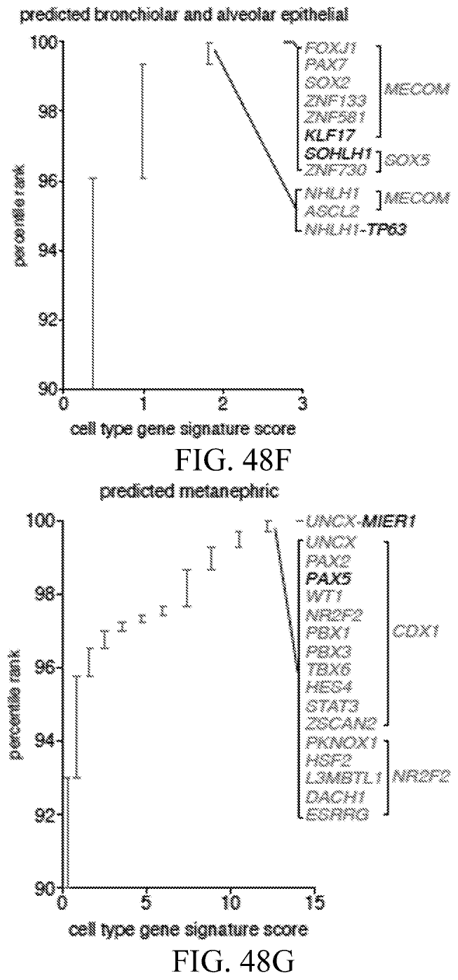

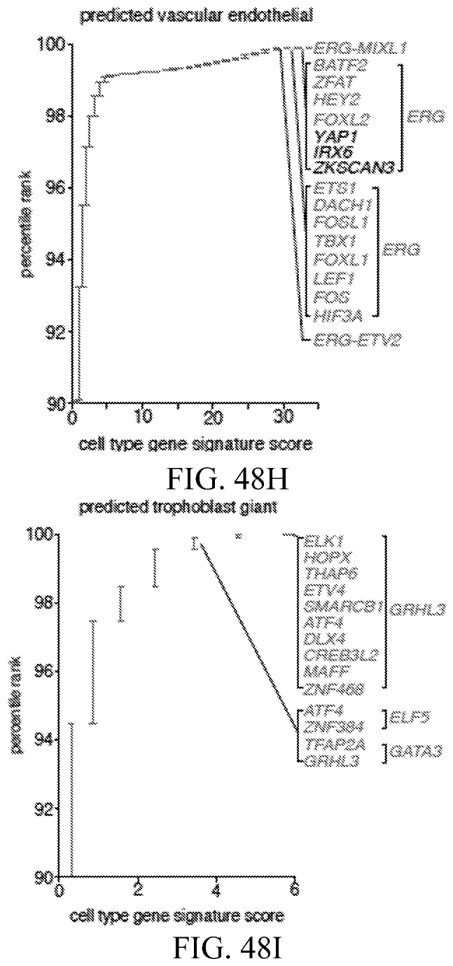

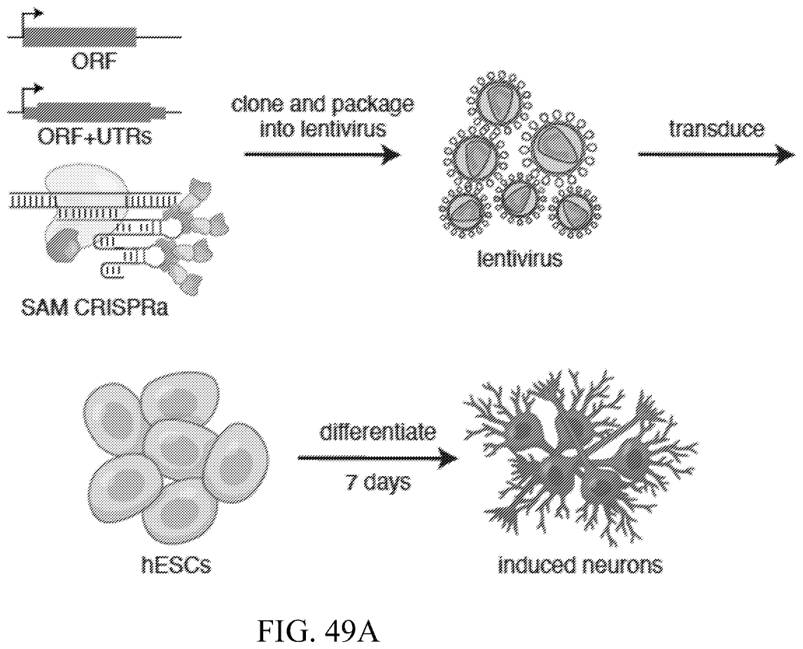

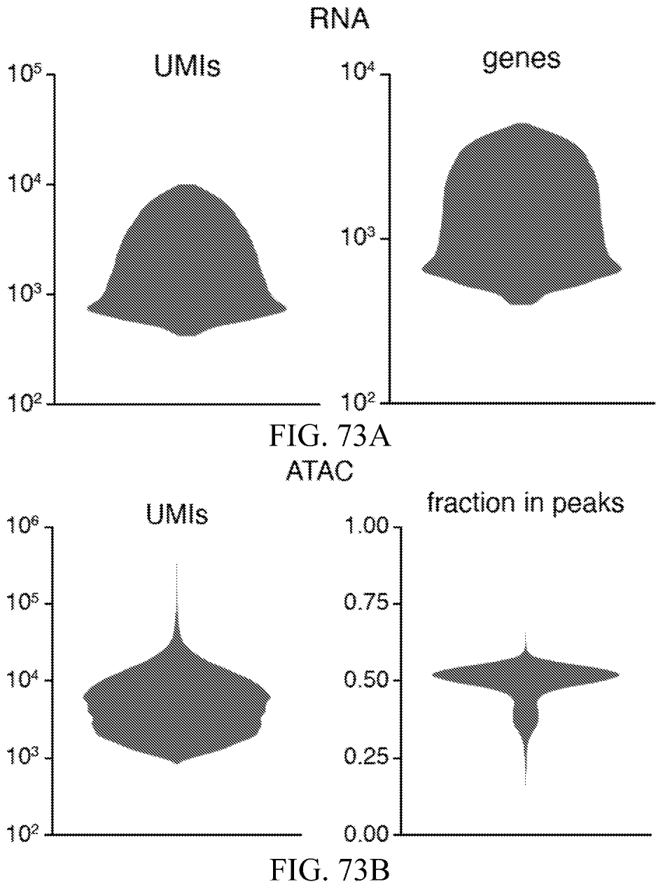

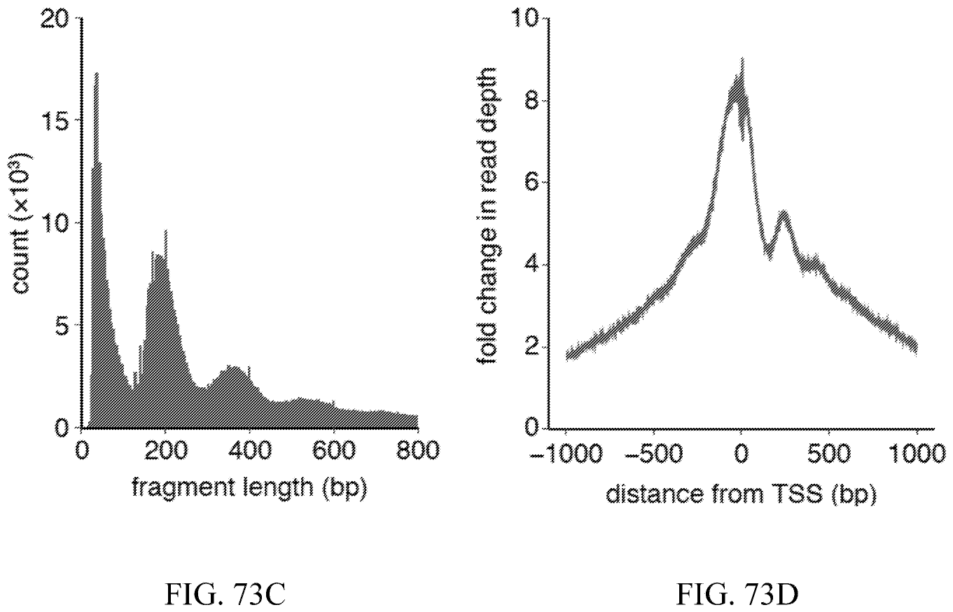

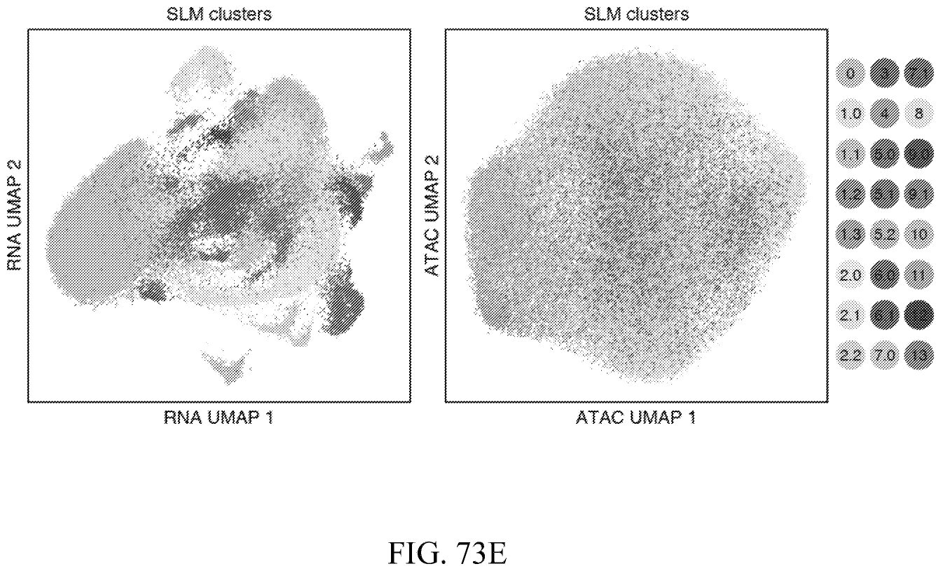

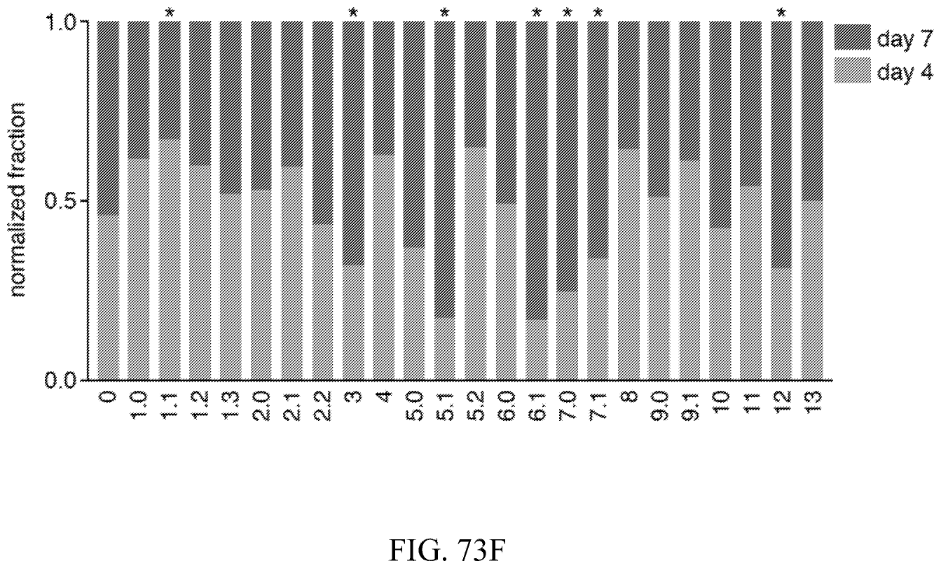

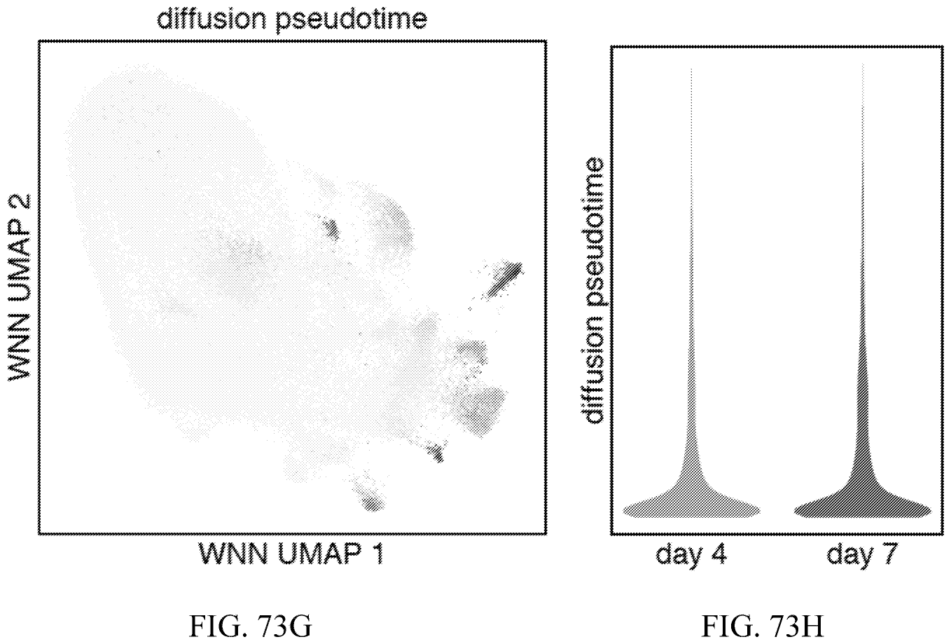

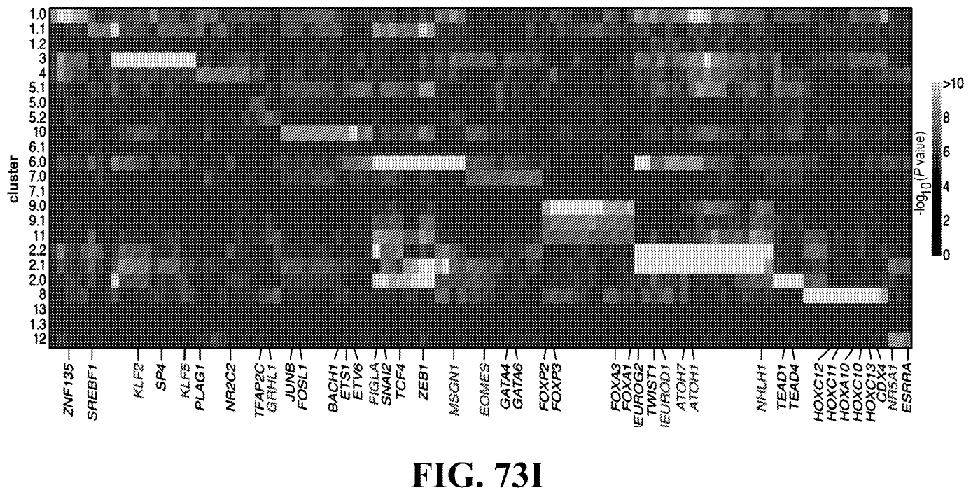

Discovery of regulatory networks by joint profiling of chromatin accessibility and expression under TF overexpression. To decipher the interplay between TF activity and chromatin state in the context of causal TF overexpression, Applicants profiled 198 diverse TFs in the MORF library by joint single cell chromatin accessibility (by ATAC-Seq) and RNA profiles (SHARE-Seq (39, 84)). Applicants introduced the targeted 198 TF MORF library into hESCs and, after 4 or 7 days, performed SHARE-seq (3,317 and 2,384 UMIs per cell on average for scATAC- and scRNA-seq, respectively; FIG. 73A-D). Applicants constructed a weighted nearest neighbor (WNN) graph based on a weighted combination of RNA and ATAC similarities that integrated the two profiles into a single representation for clustering and visualization (FIG. 47A) (85). However, when comparing two separate embeddings of RNA and ATAC profiles (FIG. 73E, labeled by the WNN clusters), there was much stronger separation between RNA profiles than between ATAC profiles, with some ATAC profiles of cells belonging to distinct clusters in the WNN embedding, now grouping together. This suggests that changes in expression, rather than in chromatin accessibility, may have primarily contributed to the TF-driven cell state alterations (FIG. 47A and FIG. 73E), potentially because the chromatin landscape in hESCs is highly accessible and primed for differentiation (43). Cluster and pseudotime distributions were not drastically different between time points, suggesting that most TF ORFs have altered gene expression within 4 days (FIG. 73F-H). For each cluster, Applicants identified marker genes enriched in both scATAC- and scRNA-seq profiles (FIG. 47B). Consistent with the clustering analyses (FIG. 73E), changes in gene expression, instead of chromatin accessibility, drove marker gene specificity across clusters (FIG. 47B). Although Applicants did observe a few instances in which marker genes exhibited an increase in both chromatin accessibility and gene expression (FIG. 47C), most showed little (FIG. 48D) or no (FIG. 47E) change in chromatin accessibility. Instead, the chromatin at marker gene promoters tended to be open and primed for TF binding to alter gene expression.

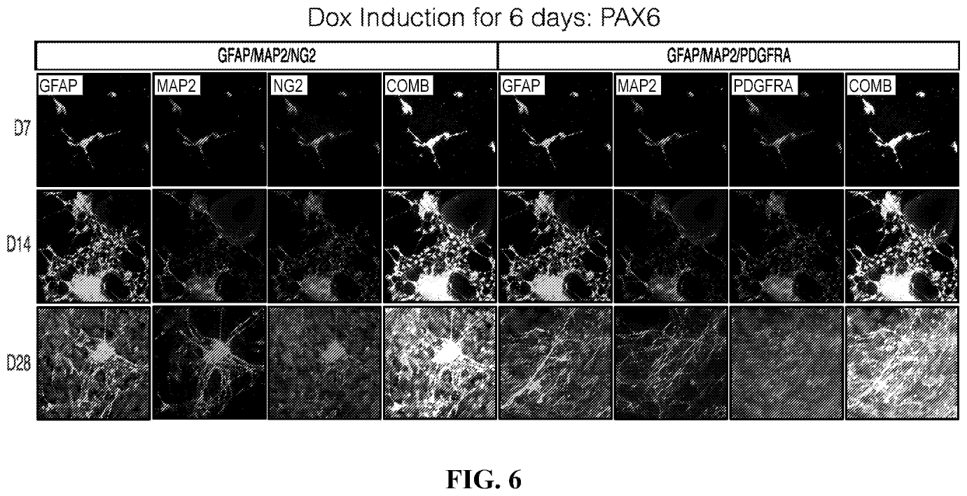

Applicants leveraged the joint chromatin accessibility and expression profiles to identify downstream TFs regulated by each TF ORF that may facilitate cell state changes. Specifically, for each cluster, Applicants nominated key putative regulators by identifying TFs with both enriched expression in the scRNA-seq and enriched accessibility for their motifs in the scATAC-seq. Applicants then mapped TF ORFs in each cluster to these respective downstream regulators (FIG. 6F). For instance, GRHL1 and GRHL3 induce upregulation of TFAP2C and TEAD family TFs, consistent with their roles in trophoblast differentiation (FIG. 44D and FIG. 47F) (86). FLI1 induces the AP-1 family of TFs (JUN and FOS) and ETV2, consistent with its ability to induce vascular endothelial cells (FIG. 44D and FIG. 47F) (87). CDX1, CDX2, and HOXD11 induced posterior Hox genes, consistent with their roles in anterior-posterior specification (FIG. 44D and FIG. 47F) (88). For 18 TF ORFs, such as KLF5, GRHL1, MSGN1, and NHLH1, the endogenous TF itself was nominated as the top regulator, suggesting a positive feedback mechanism that enhances TF expression. The MORF library design allows this distinction, because expression of the overexpressed ORF is not captured by 3′ scRNA-seq (FIG. 50A). A complementary approach to identify top downstream regulators based on TF motif enrichment in significantly functional ATAC peaks (significant correlation between chromatin accessibility and expression of neighboring genes) yielded similar relationships (FIG. 731).

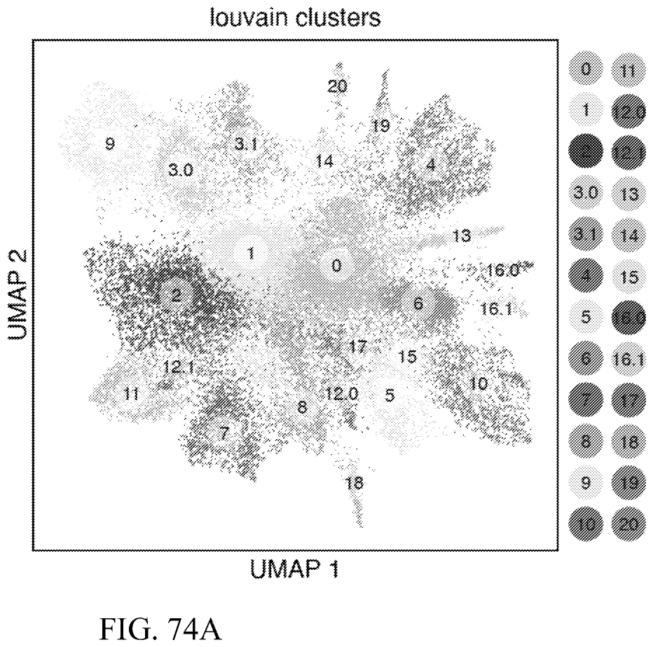

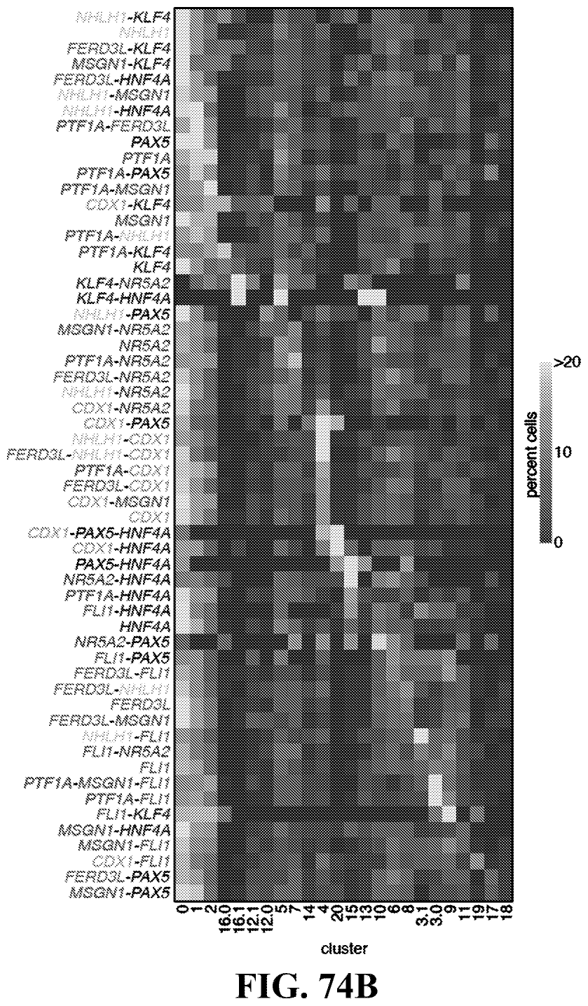

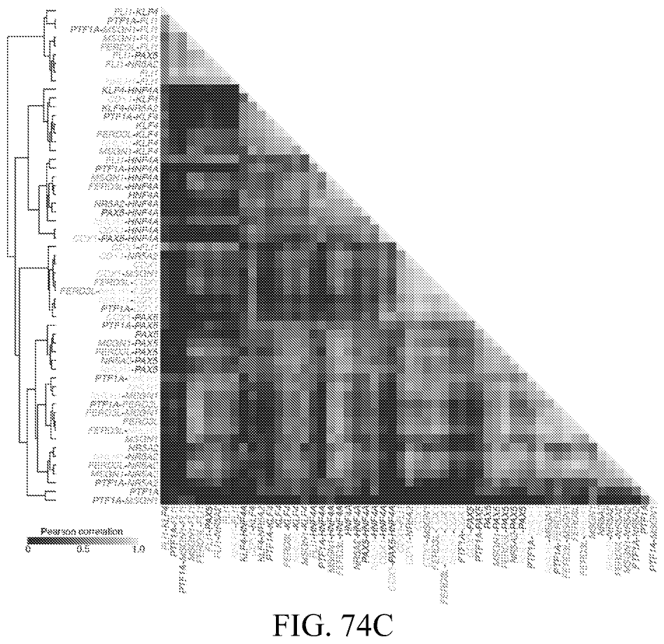

Combinatorial TF screening and prediction. Finally, as differentiation into more mature cell types often requires multiple TFs, Applicants explored how TF ORFs combine to produce the resulting expression state. To model combinatorial TF overexpression, Applicants first generated a scRNA-seq dataset for 10 TF ORFs in combinations, including 44 of the 45 possible doubles (the remaining pair was not detected) and 3 triples, as well as 10 singles (TF and GFP) as controls. Low dimensionality embedding and cluster analysis showed that expression profiles of combinations with similar TFs often grouped together, such that TF combinations were in some cases grouped with the single TF profile of one member of the respective pair, but not the other (FIG. 48A and FIG. 74), yielding a grouping of TF combinations associated with that single TF profile (e.g., CDX1, FLI1, and KLF4). In some other cases, two TFs generated a continuum of combinations, first centered around one, followed by their combination, and then centered around the other (e.g., FERD3L and NR5A2). This suggested that some TF effects may dominate those of others, whereas in other cases, the relationship may be more additive (in either nominal terms or in a non-linear embedded space).

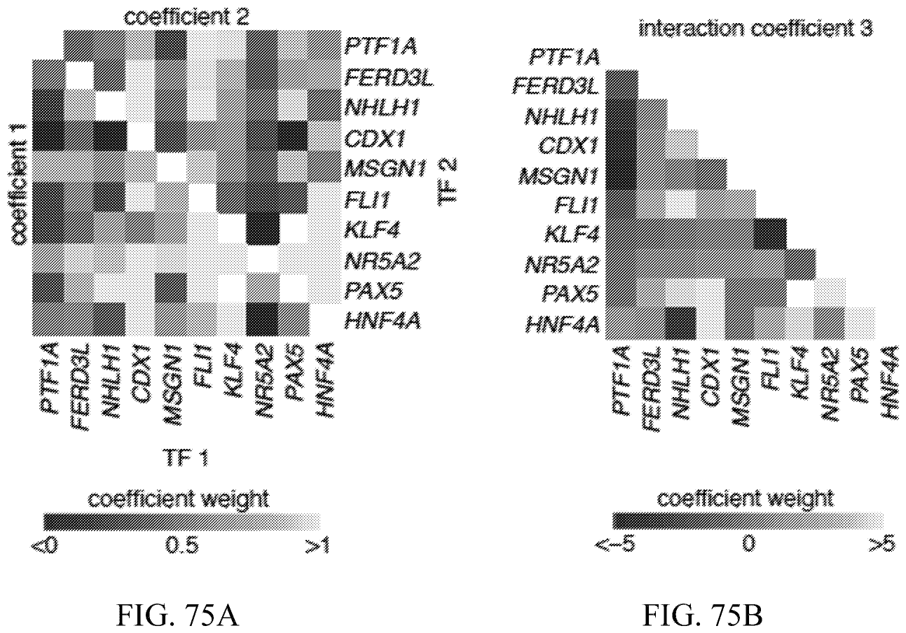

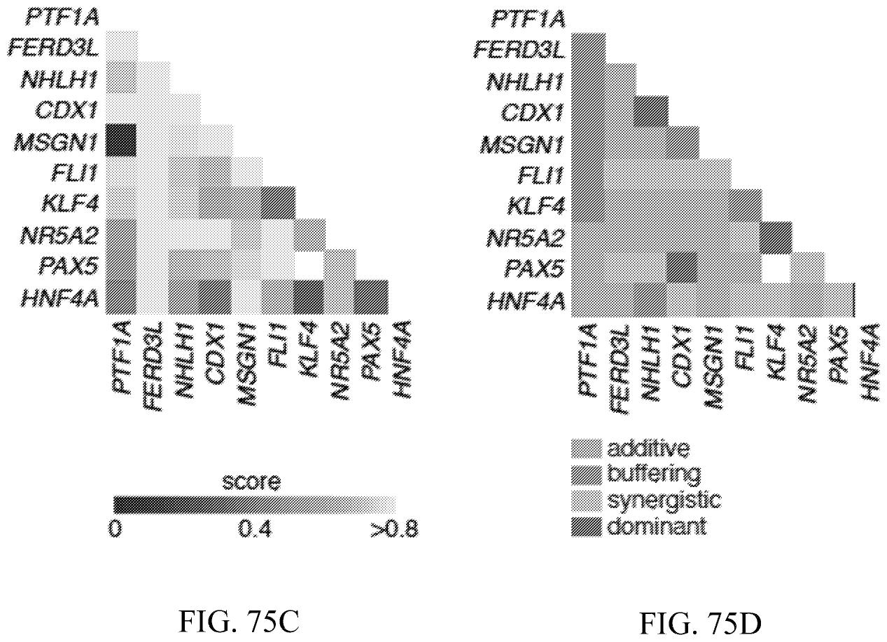

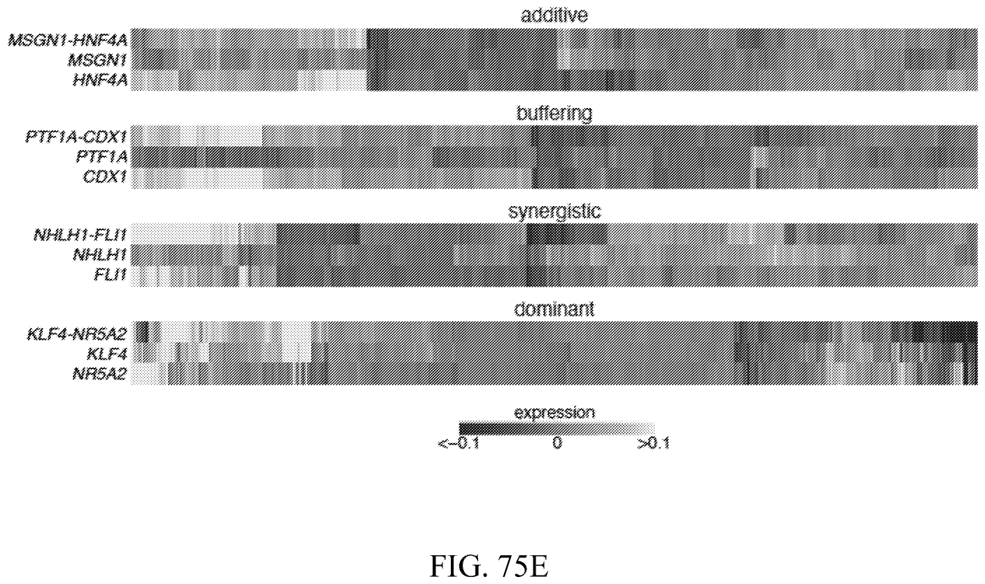

Applicants next quantitively modeled TF interactions for every gene, by a linear regression model (ab=c1*a+c2*b+c3*a*b) that fits the expression profiles observed in cells overexpressing two TFs (ab) as a linear combination of the profiles in cells overexpressing one TF (a and b) along with an interaction term (a*b) (89, 90). This linear model explained, on average, 68% of the variance in gene expression (mean R2=0.68; FIG. 75A-C). For each TF combination, Applicants used the model to assess whether their relationship was overall additive, synergistic, buffering (antagonistic), or dominant (FIG. 75D-E) when aggregating their effects across targets (as their effects can vary for each gene target). Although most TF combinations were broadly additive, Applicants identified TFs that tended to interact non-additively with other TFs in a consistent manner. For instance, combinations with PTF1A had mostly buffering effects, FLI1 was enriched for synergistic effects, and CDX1 was often dominant (FIG. 75D-E).

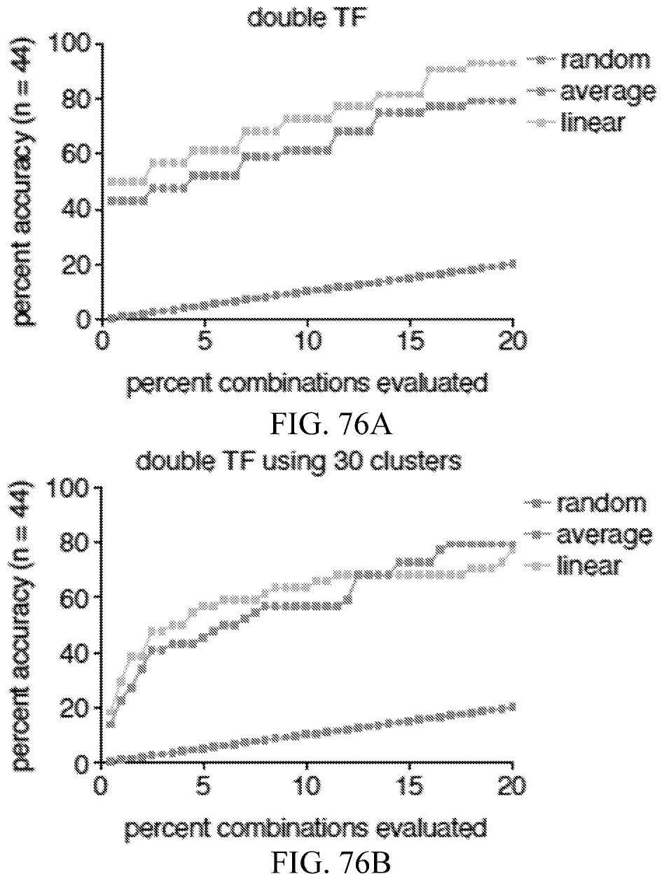

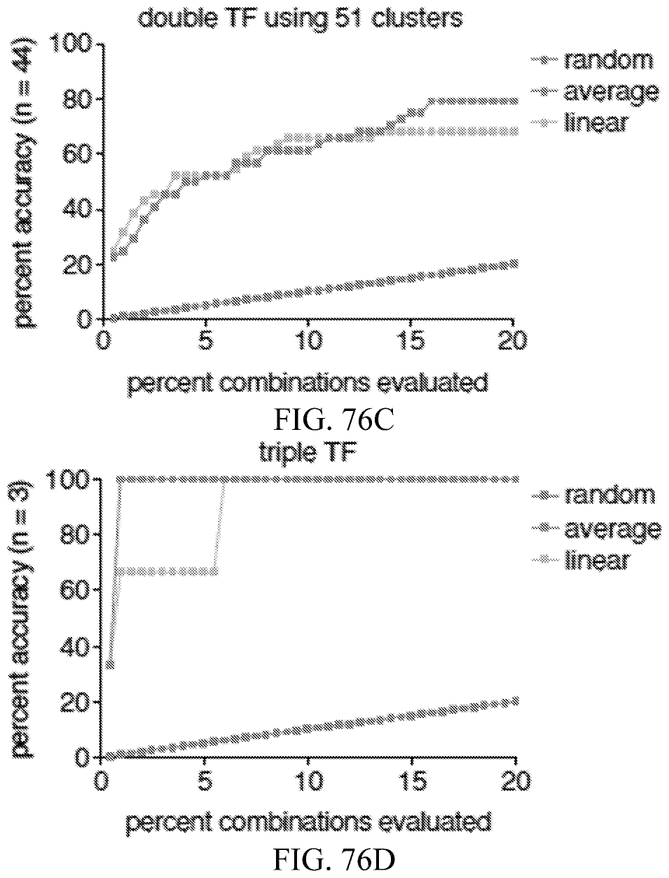

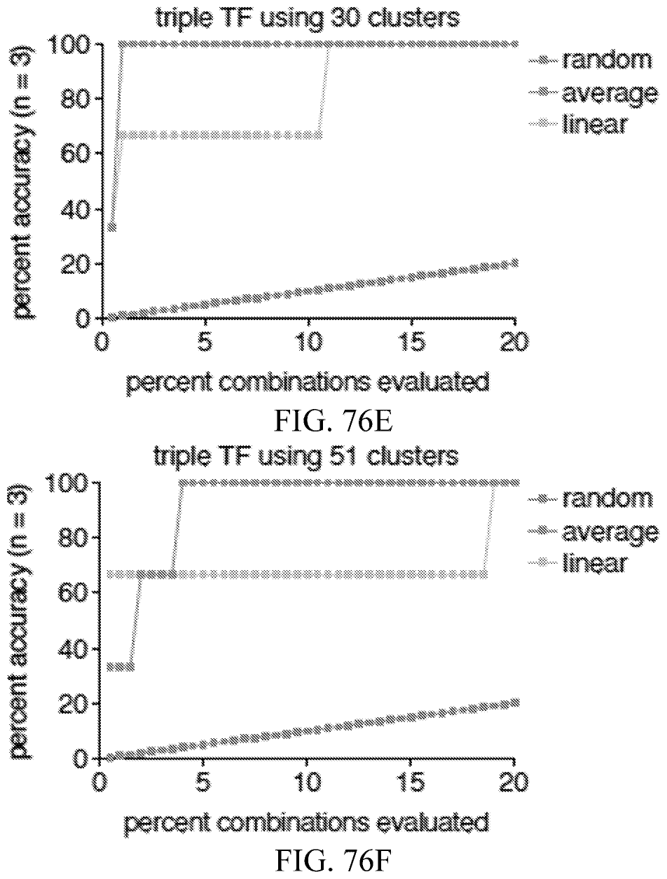

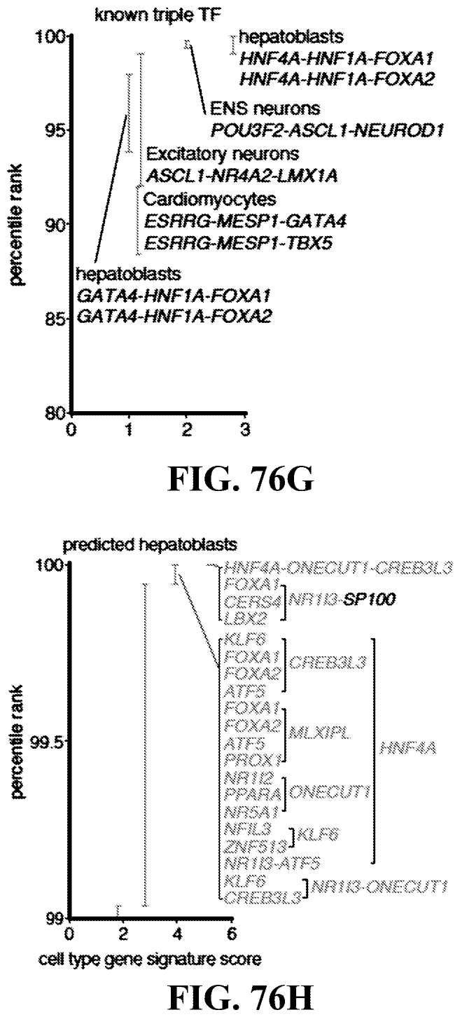

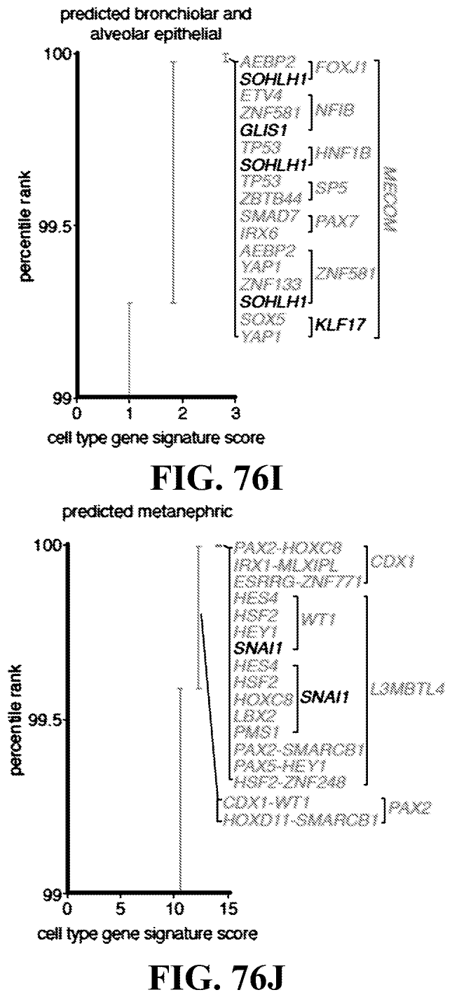

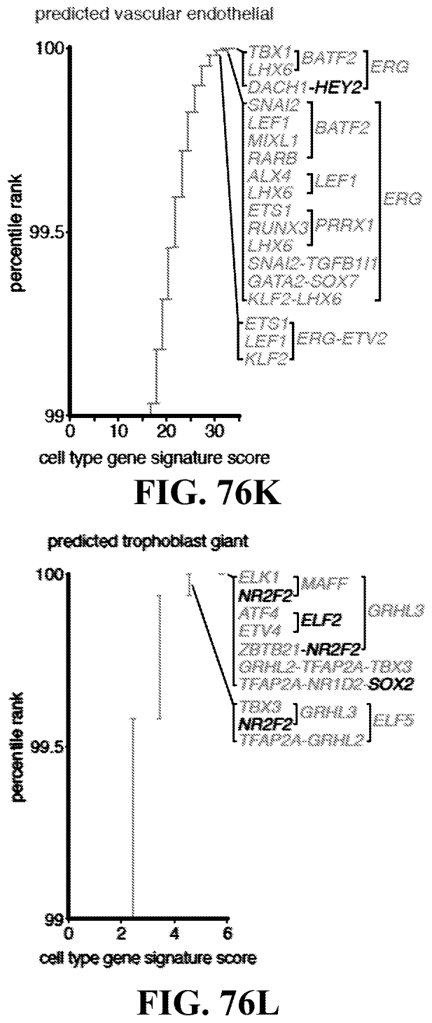

Applicants then used the combinatorial dataset to nominate TF combinations that could produce a measured combinatorial expression profile. For each measured mean combinatorial profile for TFs A and B, Applicants nominated possible combinations of any pair of TFs X and Y, based on how well X and Y's respective single TF profiles when combined fit the measured combinatorial profile of A and B. Applicants tested different approaches for combining and fitting, including taking the average or using linear and nonlinear (kernel ridge and random forest) regression methods. As the baseline, Applicants randomly selected TF combinations from the same set of possible combinations (i.e., 45 combinations for doubles and 120 combinations for triples). Applicants compared the nominated and known TF combinations to evaluate prediction accuracy. Surprisingly, simply computing the average of the single TF profiles outperformed linear and nonlinear regression approaches (FIG. 48B, C). Averaging single TF profiles to predict double TF profiles had an accuracy of 81% when evaluating only the top TF combination and 91% when evaluating the top 10% of possible TF combinations (i.e., top 4 out of 45 total combinations; FIG. 48B). For triple TF profiles, averaging correctly predicted all 3 sets of TFs when evaluating the top ˜2% of possible TF combinations (i.e., top 2 out of 120 total combinations; FIG. 48C). The relatively worse performance of linear and nonlinear regression approaches may be because most TF combinations were additive (FIG. 75D). Applicants then extended this approach by using single TF profiles from the TF Atlas for fitting and testing if Applicants can recover the correct combination of TFs that generated the measured combinatorial profile. Applicants found that Applicants could still predict both double and triple TF combinations, though with lower accuracy (FIG. 76A-F). Double TF prediction had an accuracy of 57% when evaluating the top 10% of possible TF combinations and 80% when evaluating the top 20% (FIG. 76A-C). Triple TF prediction correctly identified all 3 sets of TFs when evaluating the top 5% of possible TF combinations (FIG. 76D-F). Both double and triple TF prediction outperformed the null of randomly selecting TF combinations from the corresponding set of possible combinations.

Applicants leveraged Applicants' findings from the combinatorial dataset to develop an approach for nominating TF combinations that could differentiate hESCs into different cell types. As averaging single TF expression profiles gave us a relatively good approximation for their combinatorial TF profile, Applicants estimated the profile of all possible double and triple TF combinations by averaging single TF profiles from the TF Atlas. Applicants then scored each combinatorial TF profile for enrichment of cell type-specific gene signatures using the human fetal cell atlas (48). For each cell type, Applicants ranked potential TF combinations by the respective gene signature score. Applicants confirmed that Applicants' approach enriched for experimentally validated double TF combinations for the respective cell types, such as hepatoblasts (HNF4A and FOXA1) (91), astrocytes (SOX9 and NFIB) (92), and inhibitory neurons (ASCL1 and DLX2) (93), within the 80th percentile (FIG. 48D). Moreover, the top predicted combinations for each cell type included TFs that are part of experimentally validated combinations as well as TFs that are developmentally critical, suggesting that the TF drives core gene programs for the cell type (FIG. 48E-I and Table 19). As an example, KLF6, unlike HNF4A and HNF1B, has not been applied to hepatocyte differentiation, even though Klf6 knockout mice do not develop a liver (FIG. 48E) (94). Similarly, ERG has not been previously used for endothelial cell differentiation, but it is required for angiogenesis (FIG. 48H) (95). As expected, TF combinations enriched for TFs that Applicants experimentally validated, such as NHLH1 for bronchiolar and alveolar epithelial-like cells, CDX1 for metanephric-like cells, and GRHL3 for trophoblast-like cells, suggesting combinations that could further improve the efficiency and fidelity of differentiating these cell types (FIG. 48F-I). Similarly, for triple TF combinations, Applicants' approach enriched for combinations that were experimentally validated and developmentally relevant (FIG. 76G-L and Table 19). Thus, Applicants suggest this approach may reduce the exponentially vast search space of combinatorial TF effects for follow up empirical experimentation, accelerating the pace of cellular engineering.

Discussion. To achieve a comprehensive understanding of the gene programs governed by each TF, Applicants developed a platform for systematic TF overexpression. Applicants created a comprehensive library of 3,548 human TF splice isoforms and built a TF Atlas that maps TF ORFs to corresponding expression changes. The TF Atlas allows for systematic investigation of the relationships between TFs as well as broad-spectrum findings. Applicants mapped TF-induced expression profiles to those of reference cell types and validated TFs for production of cell types from all three germ layers and trophoblasts. Applicants then performed a targeted TF screen to establish a cellular model for neurological disorders using RFX4-derived iNPs. In a second targeted screen, Applicants integrated expression and chromatin accessibility data to identify downstream regulatory networks for a subset of TFs. Finally, Applicants generated a combinatorial TF overexpression dataset and demonstrated that Applicants could often predict the effects of combining TFs in a useful way. Applicants leveraged Applicants' findings to develop an approach for predicting TF combinations for reference cell types, which can help investigators reduce the combinatorial search space for iterative experimentation.

The accessibility and flexibility of Applicants' screening approach lends itself to scalable extensions of the technology to additional contexts. Applicants' screening approach may be applied to identify TF combinations for any cell type of interest by increasing the screening MOI to increase the probability of introducing more than one TF in the same cell (89). Iterative or sequential TF screens may also expand the landscape of possible cell types as sequential overexpression of TFs may mimic the natural developmental trajectory. For instance, one could perform a TF screen in RFX4-iNPs for more mature cell types like specific neuronal or glial cell types. In addition to identifying TFs for directed differentiation, TF screening can be used to nominate candidates involved in trans-differentiation (22, 96), as well as aging (97) and cancer progression (98). Applicants' analysis of combinations suggests that while simple additive models provide a first-generation method to predict combinations, enhanced models that perform algebra in a latent space (such as the embedding in FIG. 48A) may be able to better predict combinatorial relations, especially as those vary for individual gene targets.

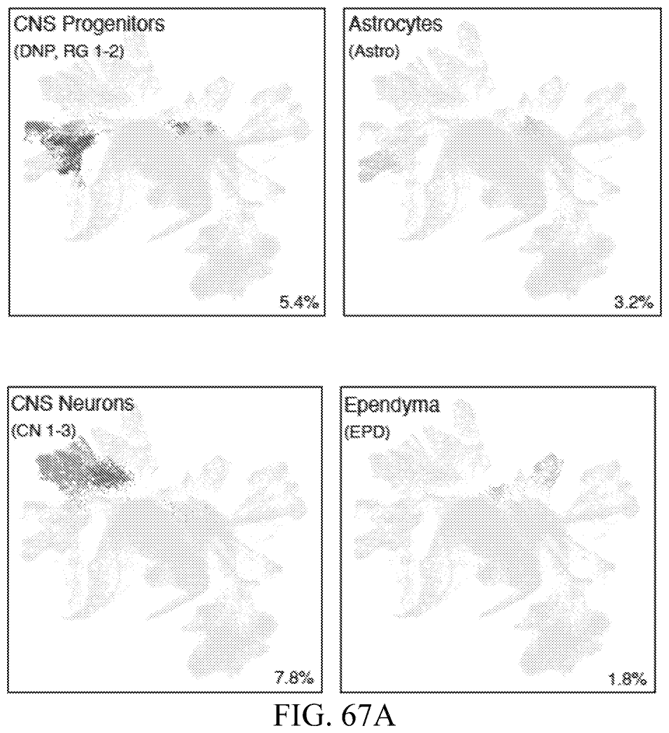

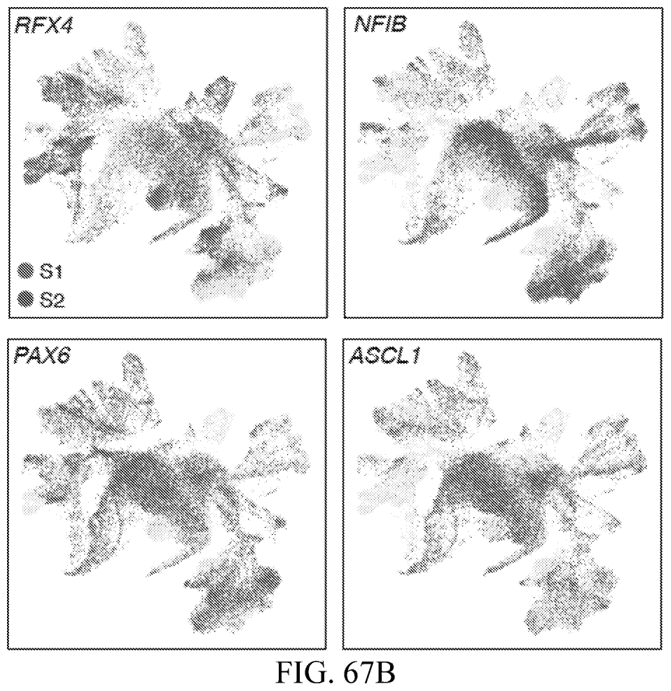

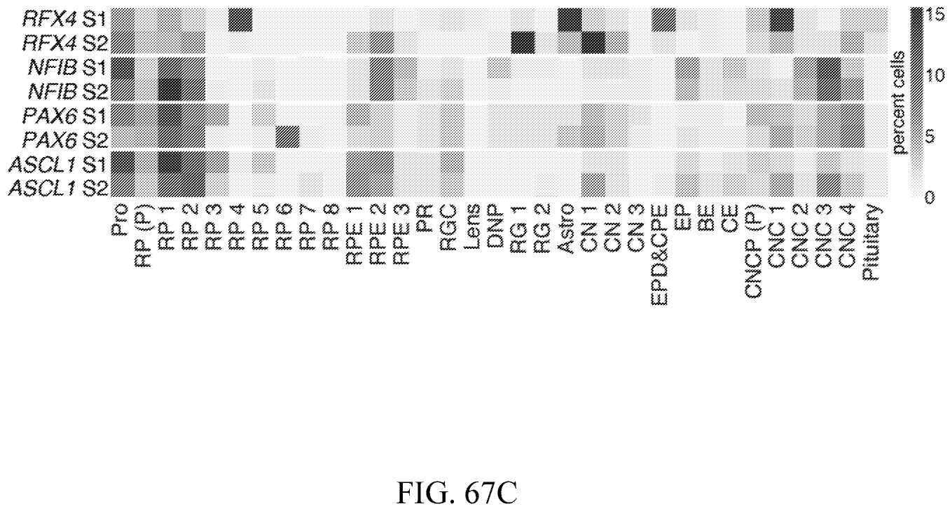

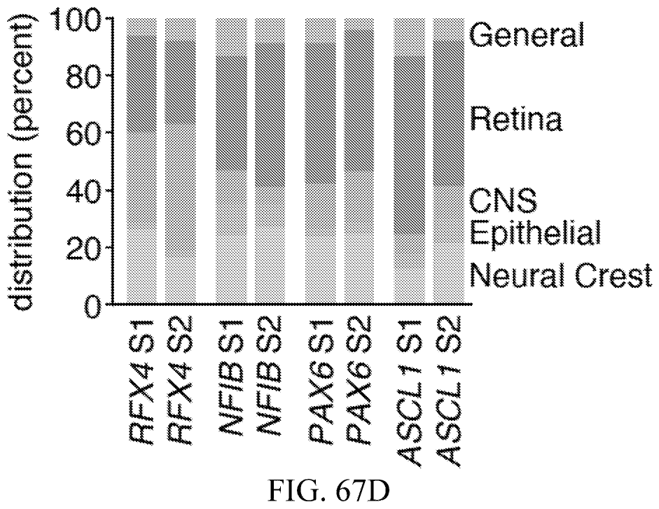

The TF Atlas establishes a framework for large-scale exploration of gene regulatory networks and, in concert with its application in other contexts such as additional cell types and differentiation time scales, will contribute to a comprehensive understanding of the processes controlling cell states. Moreover, as single cell profiling becomes more affordable, Applicants anticipate that the resolution of this TF Atlas will increase. In addition, Applicants' ORF barcoding approach allows for a variety of screening selection methods and could be extended to pooled ORF screening of other protein families of interest. Future applications of Applicants' MORF library and other pooled ORF libraries will accelerate our ability to scalably identify factors driving nearly any cellular phenotype of interest.
Note 1: Functional validation of candidate TFs by spontaneous differentiation. Applicants evaluated the multipotency of iNPs produced by candidate TFs by spontaneously differentiating the iNPs (FIG. 63A). Spontaneous differentiation of iNPs generated by four TFs (RFX4, NFIB, PAX6, and ASCL1) followed the natural developmental progression of neurogenesis starting at week 1 and proceeding to gliogenesis at week 4 (FIG. 46C and FIG. 63B, C). RFX4-iNPs patterned into neural rosettes prior to neurogenesis. Applicants validated these four candidate TFs in two additional cell lines, 11a induced pluripotent stem cells (iPSCs) and H1 hESCs. For both cell lines, overexpression of the four TFs produced iNPs that expressed higher levels of NP marker genes relative to GFP control (FIG. 64A, B). Following spontaneous differentiation, Applicants found that RFX4 and NFIB consistently produced functional iNPs in 11a iPSCs (FIG. 64C), and RFX4 produced functional iNPs in H1 hESCs (FIG. 64D). These results indicate that the effects of some TFs are cell line-dependent, while others, like RFX4, are cell line-independent, which may point to a more critical role in NP specification during development.
Note 2: Induced cardiomyocytes. During candidate TF assessment, Applicants noticed that EOMES generated cells that contracted rhythmically, a phenotype indicative of cardiomyocytes (iCM). After protocol optimization, including shortening EOMES induction to 2 days, EOMES-iCMs were comparable to iCMs generated using the canonical GSK and WNT inhibition method (GW-iCMs) (1), producing 73% and 84% TNNT2-positive cells respectively at day 10 that formed lattice-like structures (FIG. 65A-C). ScRNA-seq characterization of EOMES- and GW-iCMs at 4 weeks showed that both methods produced 72% ventricular cardiomyocytes (FIG. 65D-I). EOMES produced more smooth muscle cells, while GW produced more atrial cardiomyocytes and skeletal muscle cells (FIG. 65G-I). EOMES-iCMs expressed higher levels of maturation markers, MYL2 and HOPX (FIG. 65J, K) (2). Though a previous study showed that combining EOMES overexpression with WNT inhibition could produce iCMs (3), Applicants' results suggested that EOMES alone could generate iCMs, potentially due to the differences in EOMES splice isoforms used. These results show that TF screening can identify TFs for cell types of different lineages.
Note 3: ScRNA-seq profiling of differentiated cells from four candidate TFs. Applicants further characterized the cells spontaneously differentiated from iNPs produced by four TFs (RFX4, NFIB, PAX6, and ASCL1) using scRNA-seq. iNPs generated a broad range of cell types from the CNS, retina, epithelium, and neural crest (FIG. 66 and Table 6). For the CNS, iNPs spontaneously produced different regionally-restricted neural progenitors, as well as neurons, astrocytes, and ependyma (FIG. 67A). Applicants found that the spontaneously differentiated cell types were generally consistent between biological replicates of the same TF, except for those from RFX4-iNPs, and distinct between TFs (FIG. 67B-D). RFX4-iNPs produced more CNS cell types; NFIB-iNPs produced more epithelium and neural crest cell types; PAX6-iNPs generated diverse cell types; and ASCL1-iNPs produced more retina cell types (FIG. 67B-D). Further analysis of CNS neurons spontaneously differentiated from iNPs showed that the neurons expressed marker genes representative of diverse brain regions and neurotransmitters, including newborn cortical excitatory neurons and cortical projection neurons (FIG. 68A-D). RFX4-iNPs generated diverse neurons, NFIB-iNPs produced more cortical projection and excitatory neurons, PAX6-iNPs produced more forebrain neurons, and ASCL1-iNPs generated more forebrain GABAergic neurons (FIG. 68E). These differences potentially indicate different roles of each TF in neural development.
Note 4: ChIP-seq analysis of four candidate TFs. To better understand the transcriptional networks that lead to iNP production, Applicants profiled the four TFs using chromatin immunoprecipitation with sequencing (ChIP-seq). Motif analysis generated distinct motifs for each TF and suggested potential transcriptional coregulators, some of which have been found in previous studies (FIG. 68F) (4, 5). Applicants identified candidate genes that could contribute to iNP differentiation by examining NP marker genes with TF ChIP-seq peaks that were also differentially expressed upon TF overexpression (FIG. 68G-I). In addition, Applicants found that each of the four TFs had ChIP-seq peaks that were proximal to its own promoter, indicating a positive feedback mechanism that contributes to the high expression levels required for driving differentiation (FIG. 68H,I).

### Example 23—Materials and Methods

Sequences and cloning. The plasmids lentiMPHv2 (Addgene 89308) and lentiSAMv2 (Addgene 75112) were used for CRISPR activation. LentiCRISPRv2 (Addgene 52961) was used for CRISPR-Cas9 mediated homology-directed repair (HDR). The Puromycin resistance gene in lentiCRISPRv2 was replaced with Blasticidin resistance gene (Addgene 75112) for CRISPR-Cas9 knockout of DYRK1A. Single guide RNA (sgRNA) spacer sequences used in this study are listed in Table 11-15, and were cloned into the respective vectors as previously described (6). For spontaneous differentiation using a dox-inducible gene expression system, the plasmid pUltra-puro-RTTA3 (Addgene 58750) was used for rtTA. The EF1a promoter in pLX_TRC209 (Broad Genetic Perturbation Platform) was replaced with the pTight promoter (Addgene 31877). For DYRK1A overexpression, the codon-optimized DYRK1A sequence (NM_001396) was cloned into pLX_TRC209 (Broad Genetic Perturbation Platform) for expression under EF1a and the Hygromycin resistance gene was replaced with a Blasticidin resistance gene (Addgene 75112).

Cell culture and differentiation. HEK293FT cells (Thermo Fisher Scientific R70007) were maintained in high-glucose DMEM with GlutaMax and pyruvate (Thermo Fisher Scientific 10569010), 10% fetal bovine serum (VWR 97068-085), and 1% penicillin/streptomycin (Thermo Fisher Scientific 15140122). Cells were passaged every other day at a ratio of 1:4 or 1:5 using TrypLE Express (Thermo Fisher Scientific 12604021).

Unless otherwise specified, human embryonic stem cells (hESCs) used in these experiments were from H1 hESCs (WiCell). HUES66 hESCs (Harvard Stem Cell Institute iPS Core Facility) were used for the neural progenitor screens and candidate TF validation. Other stem cell lines used in this study include H9 hESCs (WiCell) and 11a human induced pluripotent stem cell (iPSCs) (gift from the Arlotta laboratory, Harvard University). hESCs and iPSCs were maintained in cell culture dishes coated with 1% Geltrex membrane matrix (Thermo Fisher Scientific A1413202) in mTeSR1 medium (STEMCELL Technologies 85850). For routine maintenance, stem cells were passaged 1:10-1:20 using ReLeSR (STEMCELL Technologies 05873). For lentivirus transduction and differentiation, cells were dissociated using Accutase (STEMCELL Technologies 07920) and seeded in mTeSR1 with 10 AM ROCK Inhibitor Y27632 (Enzo Life Sciences ALX-270-333-M025). All stem cells were maintained below passage 30 and confirmed to be karyotypically normal and negative for mycoplasma every 5-10 passages. Normocin (Invivogen ant-nr-1) was used as an antibiotic for stem cell culture and differentiation.

During neuronal differentiation, stem cell media was incrementally shifted towards neuronal media [Neurobasal medium (Thermo Fisher Scientific 21103049), B-27 (Thermo Fisher Scientific 17504044), and GlutaMAX (Thermo Fisher Scientific 35050061)] in 25% increments starting from day 2. On day 5, media was changed to 100% neuronal media.

During TF-driven neural progenitor (NP) differentiation, stem cell media was gradually shifted towards NP media [DMEM/F-12 with HEPES (Thermo Fisher Scientific 11330057), B-27 (Thermo Fisher Scientific 17504044), 20 ng/ml EGF (MilliporeSigma E9644), 20 ng/mL bFGF (STEMCELL Technologies 78003), and 2 μg/mL heparin (STEMCELL Technologies 07980)] in 25% increments as described above for neuronal differentiation. Cells were passaged at day 4. For spontaneous differentiation, 2 μg/ml doxycycline (MilliporeSigma D9891) was added to the media starting on day 0 for 7 days to induce TF expression. After 7 days, cells were maintained in NP media for 3 days before media was changed to differentiation media, which had the same components as NP media but without EGF and bFGF. Half of the media was refreshed every other day during spontaneous differentiation.

For RFX4-iNP protocol optimization, base media from the dual SMAD inhibition (DS) (7) and embryoid body (EB) (8) protocols were tested. DS media is a 1:1 mix of N-2 [DMEM/F12 with HEPES (Thermo Fisher Scientific 11330057), N-2 (Thermo Fisher Scientific 17502048), 5 μg/mL insulin (Millipore Sigma 19278), 100 μM nonessential amino acids (Thermo Fisher Scientific 11140050), and 100 μM 2-mercaptoethanol (Millipore Sigma M6250)] and neuronal media. EB media [DMEM/F12 with HEPES (Thermo Fisher Scientific 11330057), N-2 (Thermo Fisher Scientific 17502048), and B27 minus vitamin A (Thermo Fisher Scientific 12587010)] was also tested. SMAD inhibitors dorsomorphin (Millipore Sigma P5499) and SB-431542 (R&D Systems 1614) were added where indicated. To provide the best comparison between RFX4-iNP, DS, and EB methods, the differentiation timelines were aligned such that the iNPs produced by the three methods were dissociated for scRNA-seq at the same time.

For cardiomyocyte differentiation using EOMES-derived progenitors, HUES66 hESCs were seeded at 3×10⁵ or 5×10⁵ cells/cm². After 2 days, on day 0, 2 μg/mL doxycycline (MilliporeSigma D9891) was added to the media for 2 days unless otherwise indicated. On day 1, media was switched to cardiomyocyte differentiation media [RPMI 1640 with GlutaMax (Thermo Fisher Scientific A1895601), B-27 minus insulin (Thermo Fisher Scientific 17504044), and 10 mg/mL Ascorbic acid (Millipore Sigma A4403-100 MG)]. Media was refreshed on day 2 and every other day afterwards. On day 7, half of the media was replaced with cardiomyocyte maintenance media [RPMI 1640 with GlutaMax (Thermo Fisher Scientific A1895601) and B-27 (Thermo Fisher Scientific 17504044)]. On day 8, all of the media was replaced with cardiomyocyte maintenance media. For cardiomyocyte differentiation using GSK and Wnt inhibitors, 10 μM CHIR99021 (Selleckchem S1263) and 5 mM IWP4 (Stemgent 04-0036) were used as described previously (1).

To select the optimal media condition for the TF Atlas, stem cell media was gradually shifted towards 7 medias in 25% increments starting from day 2 as described above. Medias tested include M1 [DMEM/F-12 with HEPES (Thermo Fisher Scientific 11330057), N-2 (Thermo Fisher Scientific 17502048), B-27 (Thermo Fisher Scientific 17504044), and 100 μM nonessential amino acids (Thermo Fisher Scientific 11140050)], M2 [1:1 mix of neuronal media and DMEM/F-12 with HEPES (Thermo Fisher Scientific 11330057), N-2 (Thermo Fisher Scientific 17502048), and 100 μM nonessential amino acids (Thermo Fisher Scientific 11140050)], M3 [StemPro-34 SFM (Thermo Fisher Scientific 10639011) and GlutaMAX (Thermo Fisher Scientific 35050061)], M4 [STEMdiff APEL 2 (STEMCELL Technologies 05275)], M5 [cardiomyocyte maintenance media], M6 [KnockOut DMEM (Thermo Fisher Scientific 10829018), KnockOut Serum Replacement (Thermo Fisher Scientific 10828010), GlutaMAX (Thermo Fisher Scientific 35050061), and 100 μM nonessential amino acids (Thermo Fisher Scientific 11140050)], and M7 [stem cell media]. M4 was selected for the TF Atlas and validation.

Lentivirus production. HEK293FT cells (Thermo Fisher Scientific R70007) were cultured as described above. 1 day prior to transfection, cells were seeded at ˜40% confluency in T25, T75, or T225 flasks (Thermo Fisher Scientific 156367, 156499, or 159934). Cells were transfected the next day at ˜90-99% confluency. For each T25 flask, 3.4 μg of plasmid containing the vector of interest, 2.6 μg of psPAX2 (Addgene 12260), and 1.7 μg of pMD2.G (Addgene 12259) were transfected using 17.5 μL of Lipofectamine 3000 (Thermo Fisher Scientific L3000150), 15 μL of P3000 Enhancer (Thermo Fisher Scientific L3000150), and 1.25 mL of Opti-MEM (Thermo Fisher Scientific 31985070). Transfection parameters were scaled up linearly with flask area for T75 and T225 flasks. Media was changed 5 h after transfection. Virus supernatant was harvested 48 h post-transfection, filtered with a 0.45 μm PVDF filter (MilliporeSigma SLHV013SL), aliquoted, and stored at −80° C.

Lentivirus transduction. For transduction, 3×10⁶ hESCs or iPSCs were seeded in 10-cm cell culture dishes with an appropriate volume of lentivirus. After 24 h, media was refreshed with the appropriate antibiotic. For 5 days, media with the appropriate antibiotic was refreshed every day, and cells were passaged after 3 days of selection. Concentrations for selection agents were determined using a kill curve: 150 μg/mL Hygromycin (Thermo Fisher Scientific 10687010), 3 μg/mL Blasticidin (Thermo Fisher Scientific A1113903), and 1 μg/mL Puromycin (Thermo Fisher A1113803). Lentiviral titers were calculated by transducing cells with 5 different volumes of lentivirus and determining viability after a complete selection of 3 days (6).

qPCR quantification of transcript expression. Cells were seeded in 96-well plates and grown to 60-90% confluency before RNA was reverse transcribed for qPCR as described previously (6). TaqMan qPCR was performed with custom or readymade probes (Table 11-14).

Western blot. Protein lysates were harvested with RIPA lysis buffer (Cell Signaling Technologies 9806S) containing protease inhibitor cocktail (MilliporeSigma 05892791001). Samples were standardized for protein concentration using the Pierce BCA protein assay (VWR 23227), and incubated at 70° C. for 10 mins under reducing conditions. After denaturation, samples were separated by Bolt 4-12% Bis-Tris Plus Gels (Thermo Fisher Scientific NW04125BOX) and transferred onto a PVDF membrane using iBlot Transfer Stacks (Thermo Fisher Scientific IB401001).

For NEUROD1 and V5, blots were blocked with Odyssey Blocking Buffer (TBS; LiCOr 927-50000) for 1 h at room temperature. Blots were then probed with different primary antibodies in Odyssey Blocking Buffer overnight at 4° C. Blots were washed with TBST before incubation with secondary antibodies in Odyssey Blocking Buffer for 1 h at room temperature. Blots were washed with TBST and imaged using the Odyssey CLx (LiCOr).

For DYRK1A, blots were blocked with 5% BLOT-QuickBlocker (G Biosciences 786-011) in TBST for 1 h at room temperature. Blots were then probed with different primary antibodies in 2.5% BLOT-QuickBlocker (G Biosciences 786-011) in TBST overnight at 4° C. Blots were washed with TBST before incubation with secondary antibodies in 2.5% BLOT-QuickBlocker (G Biosciences 786-011) in TBST for 1 h at room temperature. Blots were washed with TBST and imaged using the Pierce ECL Western Blotting Substrate (Thermo Fisher Scientific 32209) on the ChemiDox XRS+(Bio-Rad).

Immunofluorescence and imaging. Cells were cultured on poly-D-lysine/laminin coated glass coverslips (VWR 354087) in 24-well plates as described above. Prior to staining, cells were washed with 1 mL PBS and fixed with 4% paraformaldehyde (VWR 15710) in PBS for 30 mins at room temperature. Cells were washed with PBS and blocked in PBS with 2.5% goat serum (Cell Signaling Technologies 5425S) and 0.1% Triton X-100 (MilliporeSigma 93443) for 1 h at room temperature. Cells were then stained with different primary antibodies in PBS with 1.25% goat serum (Cell Signaling Technologies 5425S) and 0.1% Triton X-100 (MilliporeSigma 93443) overnight at 4° C. Cells were washed in PBS with 0.1% Triton X-100 (MilliporeSigma 93443) before staining with the appropriate secondary antibodies in PBS with 1.25% goat serum (Cell Signaling Technologies 5425S) and 0.1% Triton X-100 (MilliporeSigma 93443) for 1 h at room temperature. Cells were washed in PBS with 0.1% Triton X-100 (MilliporeSigma 93443), mounted onto slides using ProLong Gold Antifade Mountant with DAPI (Thermo Fisher Scientific P36941), and nail polished (VWR 100491-940). Immunostained coverslips for NPs were imaged on a Zeiss Axio Observer with a Hamatsu Camera using a Plan-Apochromat 20× objective and a 1.6x Optovar. Immunostained coverslips for TF Atlas validation were imaged on a Leica Stellaris 5 confocal microscope using a 20× objective.

Image quantification. Images were taken from randomly selected regions using fixed exposure times. For quantification of MAP2 staining (FIG. 46I, J and FIG. 49F), the MeasureImageIntensity module in CellProfiler 3.1.8 was used to measure mean intensity on grayscale MAP2 420 μm×420 μm images. The IdentifyPrimaryObjects module in CellProfiler was used to identify and count nuclei in grayscale DAPI images with the following settings modified from default: Typical diameter of objects, in pixel units (Min, Max)=25, 70; Threshold strategy=Adaptive; Threshold smoothing scale=1.5; Lower and upper bounds on threshold=0.06, 1.0. For quantification of marker gene staining (FIG. 45D-K and FIG. 59), the MeasureImageIntensity module in CellProfiler 4.2.1 was used to measure mean intensity on grayscale 580 μm×580 μm images. The IdentifyPrimaryObjects module in CellProfiler was used to identify and count nuclei in grayscale DAPI images with the following settings modified from default: Typical diameter of objects, in pixel units (Min, Max): 25, 100; Threshold method=Otsu; Three-class thresholding; Assign pixels in the middle intensity class to the foreground; Threshold smoothing scale=5; Threshold correction factor=0.9; Lower and upper bounds on threshold=0.02, 1.0; Size of smoothing filter=10; Suppress local maxima that are closer than this minimum allowed distance=15; speed up by using lower-resolution image to find local maxima=no.

Design and cloning of TF ORF libraries. The barcoded human TF library (MORF) consisted of 1,836 genes that were selected based on AnimalTFDB (9) and Uniprot (10) annotations and included histone modifiers. The library included all 3,548 splice isoforms that overlapped between RefSeq and Gencode annotations, as well as 2 control vectors expressing GFP and mCherry. 593 of the 3,548 isoforms were obtained from the Broad Genomic Perturbation Platform and sequence verified. The rest of the isoforms were synthesized (Genewiz) and sequence verified. Table 3 lists the sequences of TFs in MORF.

To design a targeted TF ORF library for NP differentiation, single-cell or bulk RNA-seq datasets of human or mouse radial glia, neural stem cells, differentiated neural progenitors from 2D cultures or brain organoids, and fetal astrocytes were used to select TFs that were shown to be specifically expressed in these cell types (11-18). TFs that were identified in 2 or more datasets (out of 8) were included in the library. Then, bulk RNA-seq data of human fetal astrocytes (18) was used to identify TF isoforms annotated in RefSeq that comprised >25% of the TF gene transcripts. These criteria selected 90 TF isoforms covering 70 TF genes (Table 1). TF ORF isoforms that were not available from the Broad Genomic Perturbation Platform were synthesized with 24-bp barcodes (Genewiz) and cloned in an arrayed format into pLX_TRC317 (MORF; Broad Genetic Perturbation Platform) or pLX_TRC209 (targeted NP library; Broad Genetic Perturbation Platform) for expression under the EF1a promoter. Barcodes for each TF were selected to have a Hamming distance of at least 3 compared to all other barcodes.

To assess TF distribution, TF barcodes were amplified and deep-sequenced on the Illumina MiSeq or NextSeq platforms as previously described (6). For the pooled lentiviral library, lentiviral RNA was harvested using the QIAmp Viral RNA Mini Kit (Qiagen 52906) and reverse transcribed using the qScript Flex cDNA Kit (VWR 95049-100) with gene-specific priming before barcode amplification. NGS reads that perfectly matched each barcode were counted and normalized to the total number of perfectly matched NGS reads for each condition. Skew ratio was calculated as the normalized count for the 10th percentile divided by the 90th percentile.

Reporter cell line NP screen. To generate reporter cell lines, EGFP from pLX_TRC209 (Broad Genetic Perturbation Platform) followed by a T2A (GGCAGTGGAGAGGGCAGAGGAAGTCTGCTAACATGCGGTGACGT CGAGGAGAATCCTGGCCCA (SEQ ID NO: 10809)) self-cleaving peptide was inserted at the N-terminus of endogenous SLC1A3 and VIM genomic sequences. SLC1A3 and VIM were selected as NP marker genes based on convergence across published RNA-seq datasets and high expression levels (11-18). Clonal reporter cell lines were generated using CRISPR-Cas9 mediated HDR. To construct the HDR plasmids for each gene, the HDR templates that consisted of the 850-1,000 bp genomic regions flanking the sgRNA cleavage sites were PCR amplified from HUES66 genomic DNA using KAPA HiFi HotStart Readymix (KAPA Biosystems KK2602). Then EGFP-T2A flanked by HDR templates were cloned into pUC19 (Addgene 50005). HUES66 hESCs were nucleofected with 10 μg of sgRNA and Cas9 plasmid (Addgene 52961) and 6 μg of HDR plasmid using the P3 Primary Cell 4D-Nucleofector X Kit (Lonza V4XP-3024) according to the manufacturer's instructions. Cells were then seeded sparsely (2 electroporation reactions per 10-cm cell culture dish) to form single-cell clones. After 18 h, cells were selected for Cas9 expression with 0.5 μg/mL Puromycin for 2 days and expanded until colonies can be picked (˜1 week).

Cell colonies were detached by replacing the media with PBS and incubating at room temperature for 15 mins. Each cell colony was removed from the Petri dish using a 200 μL pipette tip and transferred a well in a 96-well plate for expansion. Clones with EGFP insertions were identified by 2-round PCR amplification, first with primers amplifying outside of the HDR template (HDR Fwd 1 and HDR Rev, 15 cycles) and then with primers amplifying the region of insertion (HDR Fwd 2 and HDR Rev, 15 cycles) to avoid detecting the HDR template plasmid as a false positive. Products were run on a gel to identify clones with insertions and Sanger sequencing confirmed that EGFP had been inserted at the intended site without mutations. For each reporter cell line, 3 clones with EGFP inserted into one of the two alleles were selected for further expansion and characterization.

For TF screening, SLCIA3 or VIM reporter HUES66 hESC lines were transduced with the pooled TF ORF library at MOI <0.3 and differentiated into iNPs as described above. After 7 days, 5-10×10⁶ cells were sorted for EGFP expression using the Sony SH800S Cell Sorter. For each clonal line, the percentage of cells sorted for the control condition was matched to those expressing EGFP (˜15-20%). After sorting, TF barcodes from each population were deep sequenced. Enrichment of each TF was calculated as the normalized barcode count in the high population divided by the count in the low population.

Flow-FISH NP screen. HUES66 hESCs were transduced with the pooled TF ORF library at MOI <0.3 and differentiated into iNPs as described above. After 7 days, cells were labeled with the appropriate FISH probes (Table 11-14) using the PrimeFlow RNA assay kit (Thermo Fisher Scientific 88-18005-204) with 20 million cells per biological replicate. FISH probes targeting transcripts with similar expression levels were pooled together. Once the cells were labeled, the entire cell population was sorted for high or low fluorescence (15% of cells per bin), indicating an aggregate expression level of the transcripts labeled with the pooled FISH probes for the particular wavelength. After sorting, TF barcodes from each population were amplified using a modified ChIP reverse cross-linking protocol as described previously (19). Enrichment of each TF was determined as described above for the reporter cell line screen.

10× single-cell RNA sequencing (scRNA-seq) library preparation and analysis. Cells were dissociated with Accutase (STEMCELL Technologies 07920) for 10 mins (NP) or 50 mins (spontaneously differentiated cells) at 37° C. and filtered using a 70 μm cell strainer (MilliporeSigma CLS431751) to obtain single cells. Cells were loaded in the 10x Genomics Chromium Controller with 10,000 cells per channel. For cells from the scRNA-seq pooled screen and spontaneous differentiation of four candidate TFs, scRNA-seq libraries were prepared using the Chromium Single Cell 3′ Library & Gel Bead Kit v2 (10× Genomics 120237) according to the manufacturer's instructions. Libraries were sequenced on the NextSeq platform, aiming for a minimum coverage of 20,000 reads per single cell (paired-end; read 1: 26 cycles; i7 index: 8 cycles, i5 index: 0 cycles; read 2: 55 cycles). For cells from the NP method comparison and spontaneous differentiation of RFX4-DS-iNPs, scRNA-seq libraries were prepared using the Chromium Single Cell 3′ Library & Gel Bead Kit v3 (10x Genomics 1000075) and sequenced on the HiSeq X platform (paired-end; read 1: 28 cycles; i7 index: 8 cycles, i5 index: 0 cycles; read 2: 96 cycles).

Sequencing data were aligned and quantified using the Cell Ranger Single-Cell Software Suite v3.1.0 (10× Genomics) (20) against the GRCh38 human reference genome provided by Cell Ranger. Scanpy v1.4.4 (21) was used to cluster and visualize cells. Cells with 400-7,000 detected genes and less than 10% total mitochondrial gene expression were retained for analysis. Genes that were detected in fewer than 3 cells were removed. Scanpy was used to log normalize, scale, and center the data and unwanted variation was removed by regressing out the number of UMIs and percent mitochondrial reads. Next, highly variable genes were identified and used as input for dimensionality reduction via principal component analysis (PCA). The resulting principal components were then used to cluster the cells, which were visualized using Uniform manifold approximation and projection (UMAP). Clusters were identified using Louvain by fitting the top 50 principal components to compute a neighborhood graph of observations with local neighborhood number of 20 using the scanpy.pp.neighbors function. Cells were then clustered into subgroups using the Louvain algorithm implemented as the scanpy.tl.louvain function. Cluster marker genes and associated p-values were identified using the scanpy.tl.rank_gene_groups function. To compare iNP differentiation methods, the cluster of spontaneously differentiated neurons was excluded. Intra- and inter-batch distances were calculated on the 2,305 variable genes using the spatial.distance.pdist and spatial.distance.cdist functions, respectively, from SciPy.

ScRNA-seq NP screen. HUES66 hESCs were transduced with the pooled TF ORF library at MOI <0.3 and differentiated into iNPs. Then, iNPs were dissociated for scRNA-seq analysis as described above. To pair TF barcodes with cell barcodes, TF and cell barcodes were PCR amplified from cDNA retained following the whole transcriptome amplification step of the 10× Genomics scRNA-seq library preparation protocol. The resulting amplicon was sequenced on the Illumina NextSeq platform, aiming for a minimum coverage of 20,000 reads per single cell (paired-end; read 1: 16 cycles; read 2: 72 cycles). For each cell, the TF whose corresponding barcode had the highest number of perfectly matching NGS reads was paired with the cell if the TF barcode had at least 2 reads and >25% more reads than the second highest TF. Otherwise, the cell was excluded from the scRNA-seq analysis. To identify TFs that produced similar expression profiles to radial glia, TF scRNA-seq signatures were correlated to available human fetal cortex or brain organoid scRNA-seq datasets (14, 22-25). The 1,121 most variable genes identified using the scanpy.pp.highly_variable genes function with the parameters “min_mean=0.0125, max_mean=3 and min_disp=0.5” were used. Candidate TFs were ranked based on Pearson correlations between mean expression profiles of each TF ORF and radial glia from reference datasets.

Arrayed NP screen. TF ORFs were packaged individually into lentivirus for delivery into HUES66 hESCs at MOI <0.5. After 7 days, cells were differentiated into NP and harvested for qPCR as described above to measure expression of SLC1A3 and VIM.

Bulk MORF library screen. H1 hESCs were transduced with the pooled MORF library at MOI <0.3 and differentiated for 7 days in different culture media as described above. Cells were stained for pluripotency markers, SSEA4 and TRA-1-60, and sorted for high or low fluorescence (10% of cells per bin). After sorting, TF barcodes from each population were deep sequenced. Enrichment of each TF was calculated as the normalized barcode count in the low population divided by the count in the high population.

Bulk RNA sequencing (RNA-seq) and analysis. RNA from cells plated in 24-well plates and grown to 60-90% confluency was harvested using the RNeasy Plus Mini Kit (Qiagen 74134). RNA-seq libraries were prepared using NEBNext Ultra RNA Library Prep Kit for Illumina (NEB E7530S) and deep sequenced on the Illumina NextSeq platform (>9 million reads per biological replicate). Bowtie (26) index was created based on the hg38 genome and RefSeq transcriptome. Next, RSEM v1.3.1(27) was run with command line options “--estimate-rspd --bowtie-chunkmbs 512 --paired-end” to align paired-end reads directly to this index using Bowtie and estimate expression levels in transcripts per million (TPM) based on the alignments.

TFs with similar RNA-seq signatures to reference cell types from human fetal cortex or brain organoid (14, 23, 24) were identified using Pearson correlation between expression profiles. For each TF ORF, the expression signature was defined as the top 2,000 genes with the highest fold change relative to the GFP control condition. For each cell type, the average expression profile in TPM was used. To identify genes that were differentially expressed, TPM values were log-transformed (log₂(TPM+1)) and filtered for genes that were detectable (above or equal to 1) in either condition. TF overexpression conditions were compared to control conditions using the Student's t-test. Only genes that were significant (FDR<0.05) were reported.

Chromatin immunoprecipitation with sequencing (ChIP-seq). Cells were plated in 10-cm cell culture dishes and grown to 60-80% confluency. For each condition, two biological replicates were harvested for ChIP-seq. Formaldehyde (MilliporeSigma 252549) was added directly to the growth media for a final concentration of 1% and cells were incubated at 37° C. for 10 mins to initiate chromatin fixation. Fixation was quenched by adding 2.5 M glycine (MilliporeSigma G7126) in PBS for a final concentration of 125 mM glycine and incubated at room temperature for 5 mins. Cells were then washed with ice-cold PBS, scraped, and pelleted at 1,000×g for 5 mins.

Cell pellets were prepared for ChIP-seq using the Epigenomics Alternative Mag Bead ChIP Protocol v2.0(28). Briefly, cell pellets were resuspended in 100 μL of lysis buffer (1% SDS, 10 mM EDTA, 50 mM Tris-HCL pH 8.1) containing protease inhibitor cocktail (MilliporeSigma 05892791001) and incubated for 10 mins at 4° C. Then 400 μL of dilution buffer (0.01% SDS, 1.1% Triton X-100, 1.2 mM EDTA, 16.7 mM Tris-HCl pH 8.1, and 167 mM NaCl) containing protease inhibitor cocktail (MilliporeSigma 05892791001) was added. Samples were pulse sonicated with 2 rounds of 10 mins (30s on-off cycles, high frequency) in a rotating water bath sonicator (Diagenode Bioruptor) with 5 mins on ice between each round. 10 μL of sonicated sample was set aside as input control. Then 500 μL of dilution buffer (0.01% SDS, 1.1% Triton X-100, 1.2 mM EDTA, 16.7 mM Tris-HCl pH 8.1, and 167 mM NaCl) containing protease inhibitor cocktail (MilliporeSigma 05892791001) and 1 μL of anti-V5 was added to the sonicated sample. ChIP samples were rotated end over end overnight at 4° C.

For each ChIP, 50 μL of Protein A/G Magnetic Beads (Thermo Fisher Scientific 88802) was washed with 1 mL of blocking buffer (0.5% TWEEN and 0.5% BSA in PBS) containing protease inhibitor cocktail (MilliporeSigma 05892791001) twice before resuspending in 100 μL of blocking buffer. ChIP samples were transferred to the beads and rotated end over end for 1 h at 4° C. ChIP supernatant was then removed and the beads were washed twice with 200 μL of RIPA low salt buffer (0.1% SDS, 1% Triton x-100, 1 mM EDTA, 20 mM Tris-HCl pH 8.1, 140 mM NaCl, 0.1% DOC), twice with 200 μL of RIPA high salt buffer (0.1% SDS, 1% Triton x-100, 1 mM EDTA, 20 mM Tris-HCl pH 8.1, 500 mM NaCl, 0.1% DOC), twice with 200 μL of LiCl wash buffer (250 mM LiCl, 1% NP40, 1% DOC, 1 mM EDTA, 10 mM Tris-HCl pH 8.1), and twice with 200 μL of TE (10 mM Tris-HCl pH8.0, 1 mM EDTA pH 8.0). ChIP samples were eluted with 50 μL of elution buffer (10 mM Tris-HCl pH 8.0, 5 mM EDTA, 300 mM NaCl, 0.1% SDS). 40 μL of water was added to the input control samples. 8 μL of reverse cross-linking buffer (250 mM Tris-HCl pH 6.5, 62.5 mM EDTA pH 8.0, 1.25 M NaCl, 5 mg/ml Proteinase K, 62.5 μg/ml RNAse A) was added to the ChIP and input control samples and then incubated at 65° C. for 5 h. After reverse crosslinking, samples were purified using 116 μL of SPRIselect Reagent (Beckman Coulter B23318).

ChIP-seq libraries were prepared with NEBNext Ultra II DNA Library Prep Kit for Illumina (NEB E7645S) and deep-sequenced on the Illumina NextSeq platform (>60 million reads per condition). Bowtie (26) was used to align paired-end reads to the hg38 genome with command line options q -X 300 --sam --chunkmbs 512″. Next, biological replicates were merged and Model-based Analysis of ChIP-seq (MACS) (29) was run with command line options “-g hs -B -S --mfold 6,30” to identify TF peaks. HOMER (30) was used to discover motifs in the TF peak regions identified by MACS. The findMotifsGenome.pl program from HOMER was run with the command line options “-size 200-mask” and the top 3 known and de novo motifs were presented. TFs were considered potential regulators of a candidate gene if the TF peak region identified by MACS overlapped with the 20 kb region centered around the transcriptional start site of the candidate gene RefSeq annotations.

Indel analysis. Cells plated in 96-well plates were grown to 60-80% confluency and assessed for indel rates as previously described (6). Genomic DNA was harvested from cells using QuickExtract DNA Extraction kit (Lucigen QE09050). The genomic region flanking the site of interest was amplified using NEBNext High Fidelity 2x PCR Master Mix (New England BioLabs M0541L), first with region-specific primers for 15 cycles and then with barcoded primers for 15 cycles as previously described. PCR products were sequenced on the Illumina MiSeq platform (>10,000 reads per condition), and indel analysis was performed as previously described (6).

Flow cytometry assays. To measure the proportion of cells that expressed TNNT2, cells were stained for TNNT2 (Thermo Fisher Scientific MS-295-P1; 1:200 dilution) as described previously (31). For the EdU assay, cells plated in 24-well plates were differentiated and EdU incorporation was measured using the Click-iT EdU Alexa Fluor 488 Flow Cytometry Assay Kit (Thermo Fisher Scientific C10420) according to a modified version of the manufacturer's instructions. EdU was added to the culture medium to a final concentration of 10 μM for 2 h before cells were dissociated with Accutase (STEMCELL Technologies 07920) for 15-45 mins at 37° C. Cells were transferred to a 96-well plate, pelleted at 200×g for 5 mins, and washed once with 200 μL of 1% BSA (MilliporeSigma A9418) in PBS. Cells were resuspended in 100 μL of Click-iT fixative and incubated for 15 mins at room temperature in the dark. After fixing, cells were washed with 200 μL of 1% BSA (MilliporeSigma A9418) in PBS twice, resuspended in 100 μL of Click-iT saponin-based permeabilization and wash reagent, and incubated for 15 mins in the dark. To each sample, 500 μL of Click-iT reaction cocktail was added and the reaction mixture was incubated for 30 mins at room temperature in the dark. Cells were washed with 200 μL of Click-iT saponin-based permeabilization and wash reagent twice and resuspended in 200 μL of 1% BSA (MilliporeSigma A9418) in PBS. For each sample, 10,000 cells were analyzed on a CytoFLEX Flow Cytometer (Beckman Coulter) and quantified with FlowJo (FlowJo).

Electrophysiology. Whole-cell patch-clamp recordings were performed as described (doi: 10.1016/j.celrep.2018.04.066). Recording pipettes were pulled from thin-walled borosilicate glass capillary tubing (KG33, King Precision Glass, CA, USA) on a P-97 puller (Sutter Instrument, CA, USA) and had resistances of 3-5 MΩ when filled with internal solution (in mM: 128 K-gluconate, 10 HEPES, 10 phosphocreatine sodium salt, 1.1 EGTA, 5 ATP magnesium salt and 0.4 GTP sodium salt, pH=7.3, 300-305 mOsm). The cultured cells were constantly perfused at a speed of 3 ml/min with the extracellular solution (119 mM NaCl, 2.3 mM KCl, 2 mM CaCl2, 1 mM MgCl2, 15 mM HEPES, 5 mM glucose, pH=7.3-7.4, Osmolarity was adjusted to 325 mOsm with sucrose). All the experiments were performed at room temperature unless otherwise specified.

Cells were visualized with a 40X water-immersion objective on an upright microscope (Olympus, Japan) equipped with IR-DIC. Recordings were made using a Multiclamp 700B amplifier (Molecular Devices, CA, USA) and Clampex 10.7 software (Molecular Devices, CA, USA). In current clamp mode, membrane potential was held at −65 mV with a Multiclamp 700B amplifier, and step currents were then injected to elicit action potentials. Subsequent analysis was performed using Clampfit 10.7 software (Molecular Devices, CA, USA). The spontaneous AMPA receptor mediated excitatory postsynaptic currents (sEPSCs) were recorded after entering whole-cell patch clamp recording mode for at least 3 min. The data were stored in a computer for subsequent offline analysis. Cells in which the series resistance (Rs) changed by >20% were excluded from data analysis. In addition, cells with Rs more than 20 MS2 at any time during the recordings were discarded.

TF Atlas SHARE-seq library preparation. For single TF overexpression, H1 hESCs were transduced with the pooled MORF library at MOI <0.3. For combinatorial TF overexpression, H1 hESCs were transduced with combinations of 2 or 3 TFs at MOI <1 in an arrayed format and selected with multiple antibiotics. Cells were pooled during passaging at day 4. Cells were differentiated for 7 days in STEMdiff APEL 2 (STEMCELL Technologies 05275) as described above. Cells were dissociated with Accutase (STEMCELL Technologies 07920) for 10 mins at 37° C. and filtered using a 70 μm cell strainer (MilliporeSigma CLS431751) to obtain single cells.

SHARE-seq libraries were prepared as previously described (32). Briefly, cells were fixed and permeabilized. For joint measurements of single-cell chromatin accessibility and expression (scATAC- and scRNA-seq), cells were first transposed by Tn5 transposase to mark regions of open chromatin. The mRNA was reverse transcribed using a poly(T) primer containing a unique molecular identifier (UMI) and a biotin tag. Permeabilized cells were distributed in a 96-well plate to hybridize well-specific barcoded oligonucleotides to transposed chromatin fragments and poly(T) cDNA. Hybridization was repeated three times to expand the barcoding space and ligate cell barcodes to cDNA and chromatin fragments. Reverse crosslinking was performed to release barcoded molecules. cDNA was separated from chromatin using streptavidin beads, and each library was prepared separately for sequencing. Libraries were sequenced on the Illumina NovaSeq platform, aiming for a minimum coverage of 20,000 reads per single cell (for scRNA-seq only, read 1: 100 cycles, read 2: 10 cycles, index 1: 99 cycles, index 2: 8 cycles; for scATAC- and RNA-seq, read 1: 50 cycles, read 2: 50 cycles, index 1: 99 cycles, index 2: 8 cycles). To pair TF barcodes with cell barcodes, TF and cell barcodes were PCR amplified from cDNA retained following the whole transcriptome amplification step and before tagmentation. The resulting amplicon was sequenced on the Illumina NovaSeq platform, aiming for a minimum coverage of 10,000 reads per single cell (read 1: 65 cycles; index 1: 99 cycles).

SHARE-seq data preprocessing. SHARE-seq libraries were aligned as previously described (32). Briefly, SHARE-ATAC-seq reads were trimmed and aligned to the hg38 genome using bowtie2. Reads were demultiplexed using four sets of 8-bp barcodes in the index reads, tolerating one mismatched base per barcode. Reads mapping to the mitochondria and chrY were discarded. Duplicates were removed using Picard tools (http://broadinstitute.github.io/picard/). Open chromatin region peaks were called on individual samples using MACS2 peak caller (29). Peaks from all samples were merged and peaks overlapping with ENCODE blacklisted regions (https://sites.google.com/site/anshulkundaje/projects/blacklists) were filtered out. Peak summits were extended by 150 bp on each side and defined as accessible regions. The fragment counts in peaks and TF scores were calculated using chromVAR (33).

SHARE-RNA-seq reads were trimmed and filtered for reads that contain TTTTTT at the 11-16 bases of read 2 allowing for one mismatch. Reads were aligned to hg38 genome using STAR (34). Reads were demultiplexed as described above for SHARE-ATAC-seq. Aligned reads were annotated to both exons and introns using featurecounts (35). UMI-Tools (36) was used to collapse UMIs that were within one mismatch of another UMI. UMIs with only one read were removed as potential ambient RNA contamination. A matrix of gene counts by cell was created with UMI-Tools. Cells that expressed <7,000 genes and <5% mitochondrial reads were retained. The minimum number of genes per cell was selected based on the distribution for each dataset (TF Atlas, >700 genes; joint scRNA- and ATAC-seq, >400 genes; combinatorial TF screen, >500 genes) Scanpy v1.7.2 (21) was used to preprocess the count matrix and cluster cells as described above for 10X scRNA-seq libraries. Harmony (37) (max_iter_harmony=30, max_iter_kmeans=50) was used for batch correction. To map TF ORFs to single cells, TF barcodes were ranked by number of perfectly matching NGS reads and filtered for >10 reads. For single TF overexpression, the TF barcode with the highest number of NGS reads and >50% more reads than the second highest TF were mapped. For combinatorial TF overexpression, the top two or three TF barcodes were mapped. Cells without mapped TF ORFs were excluded from downstream analyses. Scanpy's sc.pp.subsample was used to subsample datasets by TF ORF.

Pseudotime analysis. Three approaches were used to order cells along pseudotime: diffusion, RNA velocity, and Monocle3. Diffusion pseudotime was determined using Scanpy's sc.tl.diffmap (n_comps=15) and sc.tl.dpt functions. RNA velocity pseudotime was determined using scVelo (38). The top 5,000 most dispersed genes were used to estimate velocity (mode=‘stochastic’) and the velocity pseudotime function was used to determine pseudotime. Monocle3 (39) pseudotime was determined by clustering cells into the same partition and applying the order_cells function. For each approach, pseudotimes were computed using each cell expressing GFP or mCherry controls as the root cells and averaged. Genes that were differentially expressed over pseudotime were identified by fitting a linear regression on the raw counts against pseudotime using scipy.stats.linregress. Genes with slopes that were significantly different than 0 (FDR<0.05) were considered differentially expressed.

Pathway enrichment analysis. Pathway enrichment analysis was performed using g:Profiler (40). The top 100 differentially expressed genes over diffusion pseudotime or genes with the highest NMF gene program weights were provided as a ranked list for input. GO:BP pathways with between 5 and 500 genes that were significantly enriched (FDR<0.05) were included. To identify non-overlapping pathways, the enriched pathways were sorted by FDR and any pathway that had more than 50% genes overlapping was excluded. For the subset of differentiated cells in FIG. 56C, Enrichr (41) implemented by GSEApy was used to evaluate the enrichment of each pathway in the set of differentially expressed genes for each cluster.

Non-negative matrix factorization. To identify gene programs in the scRNA-seq data, non-negative matrix factorization (NMF) was performed using scikit-learn (http://scikit-learn.sourceforge.net; tol=1e-5, max_iter=10000). The analysis was performed on log-normalized, centered expression data for the set of variable genes. Negative values were converted to zero to identify enriched gene programs. Positive values were converted to zero and the data multiplied by −1 to identify depleted gene programs. The optimal number of NMF programs for enriched and depleted gene programs was determined by performing NMF analysis over a range of K values (20, 30, 50, 100, 200). The average NMF program weights for each TF ORF were ordered by hierarchical clustering using 1-correlation coefficient as the distance and Ward's linkage. Clustering results were examined for groups of TFs with known similarities to select the best value of K. Applicants chose 50 NMF programs each for enriched and depleted gene programs.

Cell type mapping. Expression profiles of differentiated cells were mapped to those of reference cell types from the human fetal cell transcriptome atlas (42) using Seurat v4 (43). The set of common variable genes between the differentiated cells and reference dataset were used for mapping. The FindTransferAnchors function from Seurat was run with parameters: dims=1:50, k.anchor=15, k.filter=100, k.score=50, and max.features=300. Cells with a maximum prediction score >0.3 were mapped to the respective cell type.

Joint chromatin accessibility and gene expression analysis. Seurat v4 (44) was used for the joint chromatin accessibility and gene expression (scATAC- and scRNA-seq) multimodal analysis. Dimensionality reduction was performed on each dataset separately. The scRNA-seq data was normalized and variable features were retained for scaling and principle component analysis (PCA). The scATAC-seq data was normalized using term-frequency inverse-document-frequency and the top 250,000 most accessible regions were retained for latent semantic indexing (LSI). Weighted nearest neighbor analysis from Seurat (dims.list=list(1:50, 2:50), prune.SNN=1/40) was performed using the scRNA-seq PCA and scATAC-seq LSI to simultaneously cluster scATAC- and scRNA-seq data. Marker genes for each cluster were identified using Presto (45).

Two approaches were used to identify top regulators in each cluster. First, chromVAR (33) computed accessibility scores for known motifs at the single cell level. Presto (45) identified TFs whose expression and motif accessibility were both significantly enriched (FDR<0.05) in each cluster. These TFs were ranked by the average of the Presto AUC statistic to identify top regulators. Second, the ATAC peaks were filtered for those whose accessibility was significantly correlated with neighboring genes (41,376 peaks with FDR<0.25) with background correction, as described previously (32). Presto identified ATAC peaks that were significantly enriched (FDR<0.05) in each cluster. Enrichment of known TF motifs was determined by a Kolmogorov-Smimov test of the position weight matrix (PWM) scores in the cluster compared to PWM scores in GC- and accessibility-matched peaks.

Combinatorial TF prediction. The average expression profiles of TF combinations were used for prediction. All possible combinations of single TF expression profiles were fitted against each measured double or triple TF profile to select the TF combination with the best fit. Linear regression (fit_intercept=False, positive=True), kernel ridge regression (alpha=1), and random forest regression (max_depth=4, n_estimators=200) from scikit-learn (http://scikit-learn.sourceforge.net) were evaluated and scored based on the coefficient of determination. Average expression profiles were scored based on Pearson correlation. To predict double and triple TF profiles using single TF profiles from the TF Atlas, the two datasets were integrated using Harmony (37). The average expression profiles from the TF Atlas differentiated cells were fitted against each double or triple TF profile. As TFs from the TF Atlas can share similar expression profiles, average expression profiles were grouped by hierarchical clustering using 1−correlation coefficient as the distance and Ward's linkage where indicated.

To predict TF combinations for reference cell types, expression profiles of double or triple TFs were estimated using the mean expression profiles from the TF Atlas differentiated cells. Individual TF profiles were grouped by hierarchical clustering as described above into 365 clusters for double TF profiles and 151 clusters for triple TF profiles to reduce the number of combinations. Group gene signatures for reference cell types from the human fetal cell atlas (42) were extracted using CelliD (46) with default parameters. Cell type-specific gene signature scores were computed on all possible estimated expression profiles for multiple TFs. Predicted TF combinations were ranked by cell type-specific gene signature scores. Combinations that did not include any cell type-specific TFs (approximately 10-50% of combinations depending on the cell type) were eliminated. For each cell type, up to 100 TFs that were significantly enriched (FDR<0.05) based on the human fetal cell atlas (42) analysis were considered specific for that cell type. In cases with more than 100 significantly enriched TFs, the top 100 TFs with the highest expression relative to other cell types were included.

Statistics. Statistical tests were applied with the sample size listed in the text and figure legends. Sample size represents the number of independent biological replicates. Data supporting main conclusions represents results from at least two independent experiments. All graphs with error bars report mean±s.e.m. values. Two-tailed t-tests were performed unless otherwise indicated. PRISM was used for basic statistical analysis and plotting (www.graphpad.com), and the R language and programming environment (www.r-project.org) was used for the remainder of the statistical analysis. Multiple hypothesis testing correction was applied where indicated.

### REFERENCES FOR EXAMPLES 22 AND 23

1. S. A. Lambert et al., The Human Transcription Factors. Cell 172, 650-665 (2018).
2. I. Amit et al., Unbiased reconstruction of a mammalian transcriptional network mediating pathogen responses. Science 326, 257-263 (2009).
3. R. Sandberg, Entering the era of single-cell transcriptomics in biology and medicine. Nat Methods 11, 22-24 (2014).
4. S. A. Dalla Torre di Sanguinetto, J. S. Dasen, S. Arber, Transcriptional mechanisms controlling motor neuron diversity and connectivity. Curr Opin Neurobiol 18, 36-43 (2008).
5. T. Ravasi et al., An atlas of combinatorial transcriptional regulation in mouse and man. Cell 140, 744-752 (2010).
6. D. E. Cohen, D. Melton, Turning straw into gold: directing cell fate for regenerative medicine. Nat Rev Genet 12, 243-252 (2011).
7. S. T. Schafer et al., Pathological priming causes developmental gene network heterochronicity in autistic subject-derived neurons. Nat Neurosci 22, 243-255 (2019).
8. A. Colman, O. Dreesen, Pluripotent stem cells and disease modeling. Cell Stem Cell 5, 244-247 (2009).
9. G. Keller, Embryonic stem cell differentiation: emergence of a new era in biology and medicine. Genes Dev 19, 1129-1155 (2005).
10. E. Kiskinis, K. Eggan, Progress toward the clinical application of patient-specific pluripotent stem cells. J Clin Invest 120, 51-59 (2010).
11. D. A. Robinton, G. Q. Daley, The promise of induced pluripotent stem cells in research and therapy. Nature 481, 295-305 (2012).
12. S. A. Morris, G. Q. Daley, A blueprint for engineering cell fate: current technologies to reprogram cell identity. Cell Res 23, 33-48 (2013).
13. H. Okano, S. Yamanaka, iPS cell technologies: significance and applications to CNS regeneration and disease. Mol Brain 7, 22 (2014).
14. K. Saha, R. Jaenisch, Technical challenges in using human induced pluripotent stem cells to model disease. Cell Stem Cell 5, 584-595 (2009).
15. S. M. Wu, K. Hochedlinger, Harnessing the potential of induced pluripotent stem cells for regenerative medicine. Nat Cell Biol 13, 497-505 (2011).
16. S. Chen et al., A small molecule that directs differentiation of human ESCs into the pancreatic lineage. Nat Chem Biol 5, 258-265 (2009).
17. K. Hochedlinger, K. Plath, Epigenetic reprogramming and induced pluripotency. Development 136, 509-523 (2009).
18. S. C. Zhang, M. Wernig, I. D. Duncan, O. Brustle, J. A. Thomson, In vitro differentiation of transplantable neural precursors from human embryonic stem cells. Nat Biotechnol 19, 1129-1133 (2001).
19. S. M. Chambers et al., Highly efficient neural conversion of human ES and iPS cells by dual inhibition of SMAD signaling. Nat Biotechnol 27, 275-280 (2009).
20. Y. Shi, P. Kirwan, J. Smith, H. P. Robinson, F. J. Livesey, Human cerebral cortex development from pluripotent stem cells to functional excitatory synapses. Nat Neurosci 15, 477-486, S471 (2012).
21. A. H. M. Ng et al., A comprehensive library of human transcription factors for cell fate engineering. Nat Biotechnol 39, 510-519 (2021).
22. Z. P. Pang et al., Induction of human neuronal cells by defined transcription factors. Nature 476, 220-223 (2011).
23. U. Parekh et al., Mapping Cellular Reprogramming via Pooled Overexpression Screens with Paired Fitness and Single-Cell RNA-Sequencing Readout. Cell Syst 7, 548-555 e548 (2018).
24. A. L. Perrier et al., Derivation of midbrain dopamine neurons from human embryonic stem cells. Proc Natl Acad Sci USA 101, 12543-12548 (2004).
25. K. Takahashi et al., Induction of pluripotent stem cells from adult human fibroblasts by defined factors. Cell 131, 861-872 (2007).
26. H. Weintraub et al., Activation of muscle-specific genes in pigment, nerve, fat, liver, and fibroblast cell lines by forced expression of MyoD. Proc Natl Acad Sci USA 86, 5434-5438 (1989).
27. J. Yu et al., Induced pluripotent stem cell lines derived from human somatic cells. Science 318, 1917-1920 (2007).
28. Y. Zhang et al., Rapid single-step induction of functional neurons from human pluripotent stem cells. Neuron 78, 785-798 (2013).
29. J. M. Pedraza, A. van Oudenaarden, Noise propagation in gene networks. Science 307, 1965-1969 (2005).
30. S. Konermann et al., Genome-scale transcriptional activation by an engineered CRISPR-Cas9 complex. Nature 517, 583-588 (2015).
31. J. Dabney, M. Meyer, Length and GC-biases during sequencing library amplification: a comparison of various polymerase-buffer systems with ancient and modern DNA sequencing libraries. Biotechniques 52, 87-94 (2012).
32. R. Sugimura et al., Haematopoietic stem and progenitor cells from human pluripotent stem cells. Nature 545, 432-438 (2017).
33. Y. Shi, P. Kirwan, F. J. Livesey, Directed differentiation of human pluripotent stem cells to cerebral cortex neurons and neural networks. Nat Protoc 7, 1836-1846 (2012).
34. X. Lian et al., Directed cardiomyocyte differentiation from human pluripotent stem cells by modulating Wnt/beta-catenin signaling under fully defined conditions. Nat Protoc 8, 162-175 (2013).
35. Y. Hayashi et al., BMP-SMAD-ID promotes reprogramming to pluripotency by inhibiting p16/INK4A-dependent senescence. Proc Natl Acad Sci USA 113, 13057-13062 (2016).
36. W. Zhao et al., The polycomb group protein Yaf2 regulates the pluripotency of embryonic stem cells in a phosphorylation-dependent manner. J Biol Chem 293, 12793-12804 (2018).
37. J. Jiang et al., A core Klf circuitry regulates self-renewal of embryonic stem cells. Nat Cell Biol 10, 353-360 (2008).
38. J. M. Daley, P. Sung, 53BP1, BRCA1, and the choice between recombination and end joining at DNA double-strand breaks. Mol Cell Biol 34, 1380-1388 (2014).
39. S. Ma et al., Chromatin Potential Identified by Shared Single-Cell Profiling of RNA and Chromatin. Cell 183, 1103-1116 e1120 (2020).
40. S. T. Crews, J. C. Pearson, Transcriptional autoregulation in development. Curr Biol 19, R241-246 (2009).
41. J. A. Saba, K. Liakath-Ali, R. Green, F. M. Watt, Translational control of stem cell function. Nat Rev Mol Cell Biol 22, 671-690 (2021).
42. C. Mayer, I. Grummt, Ribosome biogenesis and cell growth: mTOR coordinates transcription by all three classes of nuclear RNA polymerases. Oncogene 25, 6384-6391 (2006).
43. A. Gaspar-Maia, A. Alajem, E. Meshorer, M. Ramalho-Santos, Open chromatin in pluripotency and reprogramming. Nat Rev Mol Cell Biol 12, 36-47 (2011).
44. C. Halluin et al., Habenular Neurogenesis in Zebrafish Is Regulated by a Hedgehog, Pax6 Proneural Gene Cascade. PLOS One 11, e0158210 (2016).
45. M. Bouchard, P. Pfeffer, M. Busslinger, Functional equivalence of the transcription factors Pax2 and Pax5 in mouse development. Development 127, 3703-3713 (2000).
46. N. Festuccia et al., Esrb is a direct Nanog target gene that can substitute for Nanog function in pluripotent cells. Cell Stem Cell 11, 477-490 (2012).
47. B. B. McConnell, V. W. Yang, Mammalian Kruppel-like factors in health and diseases. Physiol Rev 90, 1337-1381 (2010).
48. J. Cao et al., A human cell atlas of fetal gene expression. Science 370, (2020).
49. Y. Ono, T. Nakatani, Y. Minaki, M. Kumai, The basic helix-loop-helix transcription factor Nato3 controls neurogenic activity in mesencephalic floor plate cells. Development 137, 1897-1906 (2010).
50. Y. Li, H. Luo, T. Liu, E. Zacksenhaus, Y. Ben-David, The ets transcription factor Fli-1 in development, cancer and disease. Oncogene 34, 2022-2031 (2015).
51. A. M. Ghaleb, B. B. McConnell, K. H. Kaestner, V. W. Yang, Altered intestinal epithelial homeostasis in mice with intestine-specific deletion of the Kruppel-like factor 4 gene. Dev Biol 349, 310-320 (2011).
52. L. G. van der Flier, H. Clevers, Stem cells, self-renewal, and differentiation in the intestinal epithelium. Annu Rev Physiol 71, 241-260 (2009).
53. J. G. Camp et al., Human cerebral organoids recapitulate gene expression programs of fetal neocortex development. Proc Natl Acad Sci USA 112, 15672-15677 (2015).
54. M. B. Johnson et al., Single-cell analysis reveals transcriptional heterogeneity of neural progenitors in human cortex. Nat Neurosci 18, 637-646 (2015).
55. E. Llorens-Bobadilla et al., Single-Cell Transcriptomics Reveals a Population of Dormant Neural Stem Cells that Become Activated upon Brain Injury. Cell Stem Cell 17, 329-340 (2015).
56. A. A. Pollen et al., Molecular identity of human outer radial glia during cortical development. Cell 163, 55-67 (2015).
57. J. Shin et al., Single-Cell RNA-Seq with Waterfall Reveals Molecular Cascades underlying Adult Neurogenesis. Cell Stem Cell 17, 360-372 (2015).
58. E. R. Thomsen et al., Fixed single-cell transcriptomic characterization of human radial glial diversity. Nat Methods 13, 87-93 (2016).
59. J. Q. Wu et al., Dynamic transcriptomes during neural differentiation of human embryonic stem cells revealed by short, long, and paired-end sequencing. Proc Natl Acad Sci USA 107, 5254-5259 (2010).
60. Y. Zhang et al., Purification and Characterization of Progenitor and Mature Human Astrocytes Reveals Transcriptional and Functional Differences with Mouse. Neuron 89, 37-53 (2016).
61. B. Y. Hu et al., Neural differentiation of human induced pluripotent stem cells follows developmental principles but with variable potency. Proc Natl Acad Sci USA 107, 4335-4340 (2010).
62. C. P. Fulco et al., Activity-by-contact model of enhancer-promoter regulation from thousands of CRISPR perturbations. Nat Genet 51, 1664-1669 (2019).
63. G. Steele-Perkins et al., The transcription factor gene Nfib is essential for both lung maturation and brain development. Mol Cell Biol 25, 685-698 (2005).
64. G. D. Frantz, J. M. Weimann, M. E. Levin, S. K. McConnell, Otx1 and Otx2 define layers and regions in developing cerebral cortex and cerebellum. J Neurosci 14, 5725-5740 (1994).
65. M. Gotz, A. Stoykova, P. Gruss, Pax6 controls radial glia differentiation in the cerebral cortex. Neuron 21, 1031-1044 (1998).
66. C. Englund et al., Pax6, Tbr2, and Tbr1 are expressed sequentially by radial glia, intermediate progenitor cells, and postmitotic neurons in developing neocortex. J Neurosci 25, 247-251 (2005).
67. A. Bulfone et al., Expression pattern of the Tbr2 (Eomesodermin) gene during mouse and chick brain development. Mech Dev 84, 133-138 (1999).
68. S. Casarosa, C. Fode, F. Guillemot, Mash1 regulates neurogenesis in the ventral telencephalon. Development 126, 525-534 (1999).
69. G. Quadrato et al., Cell diversity and network dynamics in photosensitive human brain organoids. Nature 545, 48-53 (2017).
70. T. J. Nowakowski et al., Spatiotemporal gene expression trajectories reveal developmental hierarchies of the human cortex. Science 358, 1318-1323 (2017).
71. P. Fuentealba et al., Expression of COUP-TFII nuclear receptor in restricted GABAergic neuronal populations in the adult rat hippocampus. J Neurosci 30, 1595-1609 (2010).
72. G. Reinchisi, K. Ijichi, N. Glidden, I. Jakovcevski, N. Zecevic, COUP-TFII expressing interneurons in human fetal forebrain. Cereb Cortex 22, 2820-2830 (2012).
73. C. Mayer et al., Developmental diversification of cortical inhibitory interneurons. Nature 555, 457-462 (2018).
74. S. De Rubeis et al., Synaptic, transcriptional and chromatin genes disrupted in autism. Nature 515, 209-215 (2014).
75. I. Iossifov et al., The contribution of de novo coding mutations to autism spectrum disorder. Nature 515, 216-221 (2014).
76. D. J. Smith et al., Functional screening of 2 Mb of human chromosome 21q22.2 in transgenic mice implicates minibrain in learning defects associated with Down syndrome. Nat Genet 16, 28-36 (1997).
77. V. Fotaki et al., Dyrk1A haploinsufficiency affects viability and causes developmental delay and abnormal brain morphology in mice. Mol Cell Biol 22, 6636-6647 (2002).
78. X. Jin et al., In vivo Perturb-Seq reveals neuronal and glial abnormalities associated with autism risk genes. Science 370, (2020).
79. B. Hammerle et al., Transient expression of Mnb/Dyrkla couples cell cycle exit and differentiation of neuronal precursors by inducing p27KIP1 expression and suppressing NOTCH signaling. Development 138, 2543-2554 (2011).
80. J. Park et al., Dyrk1A phosphorylates p53 and inhibits proliferation of embryonic neuronal cells. J Biol Chem 285, 31895-31906 (2010).
81. O. Yabut, J. Domogauer, G. D'Arcangelo, Dyrk1A overexpression inhibits proliferation and induces premature neuronal differentiation of neural progenitor cells. J Neurosci 30, 4004-4014 (2010).
82. U. Soppa et al., The Down syndrome-related protein kinase DYRK1A phosphorylates p27(Kip1) and Cyclin D1 and induces cell cycle exit and neuronal differentiation. Cell Cycle 13, 2084-2100 (2014).
83. M. A. Lalli, D. Avey, J. D. Dougherty, J. Milbrandt, R. D. Mitra, High-throughput single-cell functional elucidation of neurodevelopmental disease-associated genes reveals convergent mechanisms altering neuronal differentiation. Genome Res 30, 1317-1331 (2020).
84. J. D. Buenrostro, P. G. Giresi, L. C. Zaba, H. Y. Chang, W. J. Greenleaf, Transposition of native chromatin for fast and sensitive epigenomic profiling of open chromatin, DNA-binding proteins and nucleosome position. Nat Methods 10, 1213-1218 (2013).
85. Y. Hao et al., Integrated analysis of multimodal single-cell data. Cell 184, 3573-3587 e3529 (2021).
86. C. Krendl et al., GATA2/3-TFAP2A/C transcription factor network couples human pluripotent stem cell differentiation to trophectoderm with repression of pluripotency. Proc Natl Acad Sci USA 114, E9579-E9588 (2017).
87. E. Dejana, A. Taddei, A. M. Randi, Foxs and Ets in the transcriptional regulation of endothelial cell differentiation and angiogenesis. Biochim Biophys Acta 1775, 298-312 (2007).
88. R. Neijts, S. Amin, C. van Rooijen, J. Deschamps, Cdx is crucial for the timing mechanism driving colinear Hox activation and defines a trunk segment in the Hox cluster topology. Dev Biol 422, 146-154 (2017).
89. A. Dixit et al., Perturb-Seq: Dissecting Molecular Circuits with Scalable Single-Cell RNA Profiling of Pooled Genetic Screens. Cell 167, 1853-1866 e1817 (2016).
90. A. P. Capaldi et al., Structure and function of a transcriptional network activated by the MAPK Hog1. Nat Genet 40, 1300-1306 (2008).
91. S. Sekiya, A. Suzuki, Direct conversion of mouse fibroblasts to hepatocyte-like cells by defined factors. Nature 475, 390-393 (2011).
92. I. Canals et al., Rapid and efficient induction of functional astrocytes from human pluripotent stem cells. Nat Methods 15, 693-696 (2018).
93. N. Yang et al., Generation of pure GABAergic neurons by transcription factor programming. Nat Methods 14, 621-628 (2017).
94. X. Zhao et al., Klf6/copeb is required for hepatic outgrowth in zebrafish and for hepatocyte specification in mouse ES cells. Dev Biol 344, 79-93 (2010).
95. G. M. Birdsey et al., The endothelial transcription factor ERG promotes vascular stability and growth through Wnt/beta-catenin signaling. Dev Cell 32, 82-96 (2015).
96. K. Song et al., Heart repair by reprogramming non-myocytes with cardiac transcription factors. Nature 485, 599-604 (2012).
97. A. Ocampo et al., In Vivo Amelioration of Age-Associated Hallmarks by Partial Reprogramming. Cell 167, 1719-1733 e1712 (2016).
98. J. E. Darnell, Jr., Transcription factors as targets for cancer therapy. Nat Rev Cancer 2, 740-749 (2002).

TABLE 16
Differentially expressed genes over pseudotime for TF Atlas. Linear regression was applied to identify genes
that were significantly differentially expressed (FDR < 0.05) over pseudotime. Genes were ranked by the
estimated slope of the linear regression fit. The top 1,000 most upregulated and downregulated genes are listed.

| | Diffusion | Diffusion | RNA Velocity | RNA Velocity | Monocle3 | Monocle3 |
|---|---|---|---|---|---|---|
| Rank | Upregulated | Downregulated | Upregulated | Downregulated | Upregulated | Downregulated |
| 1 | FBN2 | CD24 | GPC6 | PTMA | EXT1 | RPS8 |
| 2 | SLIT2 | LIN28A | IMMP2L | CD24 | PVT1 | CD24 |
| 3 | PRTG | RPS6 | EXT1 | RPS8 | PTPRK | RPL13 |
| 4 | TTN | RPL13A | SMYD3 | RPS6 | UBE2E2 | RPS6 |
| 5 | MAML2 | RPS12 | FAM155A | GAPDH | GPC6 | RPL13A |
| 6 | FN1 | SFRP1 | NLGN1 | RPL13A | KCNT2 | RPS12 |
| 7 | GPC6 | RPS8 | FBXL7 | RPLP1 | KHDRBS2 | GAPDH |
| 8 | LPP | GAPDH | UBE2E2 | EEF1A1 | PRKG1 | RPLP1 |
| 9 | CHODL | NPM1 | PRKG1 | HIST1H4C | BBS9 | RPL19 |
| 10 | EXT1 | PTMA | ST6GALNAC3 | RPL13 | GULP1 | RPS18 |
| 11 | CALD1 | EEF1A1 | SOX5 | NPM1 | TENM3 | EEF1A1 |
| 12 | NEAT1 | RPLP1 | ROBO2 | RPS18 | LSAMP | PTMA |
| 13 | FGF13 | POU5F1 | PTPRD | RPL11 | FNDC3B | RPL11 |
| 14 | SOX5 | RPL13 | LPP | RPS12 | PTPRD | RPL23 |
| 15 | PTPRM | RPS18 | DIAPH3 | LIN28A | CRIM1 | NPM1 |
| 16 | CDK14 | L1TD1 | KCNT2 | HSP90AB1 | ST6GALNAC3 | RPS27A |
| 17 | LSAMP | USP9X | WWOX | HSP90AA1 | TLE4 | RPL15 |
| 18 | MAML3 | HSP90AB1 | GRIP1 | ACTB | IMMP2L | RPL5 |
| 19 | FLRT2 | RPL11 | TENM3 | RPL19 | GPHN | RPL10A |
| 20 | TENM3 | RPL23 | KHDRBS2 | HMGA1 | TRIO | RPL3 |
| 21 | AHNAK | PODXL | SYT1 | RPL4 | KLF12 | RPS23 |
| 22 | SAMD4A | RPL3 | ZC3H12B | RPL31 | NRXN3 | TPT1 |
| 23 | RBMS3 | ESRG | DLG2 | RPL23 | SPATA5 | RPL4 |
| 24 | PALLD | RPL12 | FAT3 | RPL12 | NLGN4Y | RPL6 |
| 25 | CNTNAP2 | RPL19 | VPS13B | RPL3 | TCF4 | ACTG1 |
| 26 | PTPRD | TERF1 | LSAMP | RPL10A | PRIM2 | RPL8 |
| 27 | HAPLN1 | RPL10A | TRIO | RPS27A | KIAA1217 | RPL12 |
| 28 | NLGN1 | RPS27A | MAML2 | RPL34 | KAZN | RPL31 |
| 29 | LOC101927668 | RPSA | MBD5 | SET | FBXL7 | LIN28A |
| 30 | FNDC3B | HSPA8 | LINGO2 | HNRNPA1 | ADCY2 | HNRNPA1 |
| 31 | PDE4D | SET | PTPRK | HSPA8 | ADGRL3 | RPS3 |
| 32 | ST6GALNAC3 | HNRNPA1 | BCKDHB | RPL6 | CASC15 | HSP90AB1 |
| 33 | SSBP2 | DNMT3B | RORA | RPL15 | MBD5 | RPLPO |
| 34 | KIAA1217 | EEF2 | SPATA5 | HSPD1 | GRIP1 | RPSA |
| 35 | CCDC141 | HSPD1 | GPHN | RPS23 | COMMD10 | RACK1 |
| 36 | ADCY2 | RPLPO | NRXN3 | RPL5 | NLGN1 | RPL14 |
| 37 | ZSWIM6 | RPL5 | ARL15 | RPL8 | DIAPH3 | HSPA8 |
| 38 | TRIO | RPL15 | KAZN | RPL14 | RIMS2 | HSPD1 |
| 39 | CDH2 | RPL31 | CDK14 | RPL35A | FAF1 | RPL34 |
| 40 | 11-Sep | RPS2 | CTNNA2 | RPS24 | USP25 | RPS2 |
| 41 | P3H2 | HSP90AA1 | RAD51B | TPT1 | SMYD3 | RPL18 |
| 42 | APP | RPL34 | EFNA5 | NCL | FAM155A | HSP90AA1 |
| 43 | SMYD3 | RACK1 | FNDC3B | RPS14 | EFNA5 | RPS24 |
| 44 | AKT3 | RPL4 | SSBP2 | FTH1 | PHLPP1 | RPS14 |
| 45 | SHROOM3 | PARP1 | SPIDR | RPS19 | CACNA2D3 | RPS19 |
| 46 | VPS13B | RPL8 | FAF1 | RPL32 | TCF12 | FTL |
| 47 | GREB1L | HIST1H4C | COMMD10 | RPS3 | DLG2 | RPS3A |
| 48 | COL11A1 | HMGA1 | ADCY2 | RACK1 | KCND2 | RPS5 |
| 49 | ASAP1 | RPS23 | NRG3 | TMSB4X | B3GALT1 | RPL35A |
| 50 | COL5A2 | SERBP1 | CTNND2 | TUBB | TMTC2 | RPS15 |
| 51 | ROBO2 | RPS15 | KCND2 | RPL18 | ASAP1 | ACTB |
| 52 | TNS3 | RPS24 | EXOC4 | POU5F1 | SYT1 | RPL21 |
| 53 | DOCK4 | UGP2 | RIMS2 | EEF2 | MMP16 | RPL10 |
| 54 | NRIP1 | RPS5 | LOC100420587 | RPS15 | VPS13B | RPS15A |
| 55 | LOC100505817 | NASP | PRIM2 | RPLPO | GMDS | RPL30 |
| 56 | SPP1 | NCL | EGFEM1P | RPS15A | FMNL2 | RPS17 |
| 57 | CAMK2D | APELA | TBC1D5 | RPL7 | ARL15 | FTH1 |
| 58 | MAPK10 | RPS3 | TTC28 | RPL21 | TMEM132D | RPL7A |
| 59 | FBXL7 | RPS16 | CDKAL1 | RPL35 | RANBP17 | EEF2 |
| 60 | DIAPH3 | LDHB | PVT1 | ACTG1 | RFX3 | RPS7 |
| 61 | TMTC2 | RPS14 | NLGN4Y | RPL24 | KIAA0825 | TUBB |
| 62 | ARL15 | USP44 | FBN2 | LDHB | DOCK4 | POU5F1 |
| 63 | SYT1 | RPL21 | TTN | RPL30 | CTNND2 | RPL7 |
| 64 | TTC28 | RPS4X | SUPT3H | RPS16 | SLIT2 | RPS16 |
| 65 | MID1 | RPL35A | ASAP1 | RPSA | FOXP1 | LDHB |
| 66 | IL1RAPL1 | DPYSL3 | KLF12 | RPS7 | AUTS2 | RPS4X |
| 67 | TMTC1 | RPL18 | RANBP17 | RPL28 | FOCAD | RPS11 |
| 68 | MIR4435-2HG | RPS27 | MID1 | RPS2 | ZC3H12B | RPL28 |
| 69 | LOC644919 | RPL14 | TCF12 | RPS3A | PLXDC2 | SET |
| 70 | FER | RPS17 | PLXDC2 | PFN1 | TBC1D5 | SERBP1 |
| 71 | ADAMTS12 | SOX2 | TCF4 | FTL | LOC100420587 | RPL24 |
| 72 | PTPRG | RPS15A | GULP1 | RPS5 | PKIB | RPL32 |
| 73 | CDH11 | RPL28 | GALNT13 | RPS27 | UST | RPL27A |
| 74 | MBD5 | RPS3A | CASC15 | SERBP1 | RYR2 | TMSB4X |
| 75 | FAF1 | RPS11 | UGGT2 | RPS17 | CHD2 | RPS13 |
| 76 | PPP3CA | RPL30 | NBEA | RPL10 | OPCML | HMGA1 |
| 77 | TLE4 | TARS | RYR2 | RPL7A | FAT3 | NCL |
| 78 | TRPS1 | TPT1 | OPCML | PABPC1 | BCKDHB | PARP1 |
| 79 | TUSC3 | RPL6 | PARD3 | RPS11 | CDK14 | NUCKS1 |
| 80 | TTC3 | MFGE8 | TENM4 | RPL36 | TMEM132B | H2AFZ |
| 81 | CRIM1 | PABPC1 | ERC2 | RPS4X | LPP | EIF4G2 |
| 82 | SORBS2 | RPL7A | TMTC2 | H2AFZ | TUSC3 | RPL26 |
| 83 | KLF12 | TUBB | AKT3 | RPLP2 | RASAL2 | PABPC1 |
| 84 | CDK6 | RPS19 | TMEM132D | ENO1 | GALNT13 | RPLP2 |
| 85 | IMMP2L | PLS3 | FHIT | NUCKS1 | ROBO2 | RPS9 |
| 86 | FHIT | RPL27A | CEP128 | PARP1 | PTPRG | RPS27 |
| 87 | SPIDR | RPS7 | METTL15 | RPS13 | FAM13A | EEF1G |
| 88 | UBE2E2 | RPL10 | SLIT2 | RPL26 | PPP3CA | ENO1 |
| 89 | PRKG1 | DPPA4 | LOC644919 | RPL37 | MAML2 | TUBA1B |
| 90 | SERPINE2 | RPL7 | VTI1A | RPL27A | SETBP1 | RAN |
| 91 | DSP | RPL24 | SDK1 | L1TD1 | CDKAL1 | GSTP1 |
| 92 | ITGB1 | HNRNPA2B1 | ANK2 | SFRP1 | PARP8 | PFN1 |
| 93 | RBMS1 | RPS9 | LINC01572 | CANX | RFX7 | CANX |
| 94 | MED13L | NUCKS1 | ROBO1 | EIF4G2 | NRG3 | RPL9 |
| 95 | ANKRD1 | RPS13 | PPP3CA | HNRNPA2B1 | ANK2 | RPL29 |
| 96 | SVIL | PSIP1 | RFX3 | RAN | COG5 | MYL6 |
| 97 | COL2A1 | IPO5 | LRBA | RPS29 | LCORL | HNRNPA2B1 |
| 98 | ELMO1 | LECT1 | AGAP1 | ESRG | RERE | FAU |
| 99 | ATP2B1 | RPLP2 | DPP10 | UBB | PCNX2 | SFRP1 |
| 100 | ZEB2 | RPS20 | PTPRG | NACA | PARD3 | RPL35 |
| 101 | LAMB1 | RPL32 | TUSC3 | CALM1 | TEAD1 | H3F3B |
| 102 | APBB2 | RPL18A | GMDS | GSTP1 | LOC644919 | RPL18A |
| 103 | SETBP1 | COX3 | PKIB | RPS9 | ZMAT4 | NACA |
| 104 | DMD | CLDN6 | UST | RPL29 | UGGT2 | CBX5 |
| 105 | ZC3H12B | H2AFZ | MED13L | CBX5 | AGAP1 | CALM1 |
| 106 | RN7SK | RPL37A | ADAMTS19 | RPL37A | DENND1A | RPS20 |
| 107 | TCF12 | ACTB | MAGI2 | EEF1G | LINGO2 | UBB |
| 108 | PARP8 | PGRMC1 | EXOC6B | RPS20 | ZFAND3 | RPL37A |
| 109 | DACH1 | COX7C | AUTS2 | NASP | LDLRAD3 | SCD |
| 110 | CADM1 | FAU | PLCB4 | FAU | METTL15 | HSPA5 |
| 111 | TBC1D5 | CBX5 | FOCAD | HIST1H1B | SUPT3H | RPL36 |
| 112 | FBXL17 | HSPA4 | LOC101929378 | ND5 | PSD3 | PRDX1 |
| 113 | PTPRK | CEBPZ | BBS9 | H3F3B | NEDD4L | YWHAZ |
| 114 | EPB41L2 | CANX | ARID1B | SCD | LARGE | ESRG |
| 115 | SEMA6A | RPL9 | PLEKHA5 | COX3 | MAGI1 | YWHAE |
| 116 | FTO | EEF1G | PARD3B | RPL18A | TOX | HIST1H4C |
| 117 | EFNA5 | RPL29 | MIPOL1 | PRDX1 | PCSK5 | ATP5B |
| 118 | FIGN | RPL35 | TRPM3 | COX1 | ADGRV1 | MDH1 |
| 119 | SIPA1L2 | RAN | PTK2 | YWHAE | NSMCE2 | GJA1 |
| 120 | GLI3 | USO1 | ANKS1B | TUBA1B | MED13L | CALM2 |
| 121 | KIF26B | RPL26 | SETBP1 | MYL6 | PPM1L | RPL37 |
| 122 | UGGT2 | LITAF | LDB2 | YBX1 | UNC5D | ATP5A1 |
| 123 | RHOBTB3 | RPL36 | TBCK | RPL9 | SDK1 | L1TD1 |
| 124 | NTS | ATP6 | RASAL2 | ATP5B | LOC102467213 | HNRNPA3 |
| 125 | CBLB | SLC16A1 | DOCK4 | SOX2 | SOX5 | PPIA |
| 126 | GTDC1 | HNRNPU | ZFAND3 | HSPA5 | ERC2 | HSPA4 |
| 127 | ROR2 | RPL37 | CACNA2D1 | HSP90B1 | HS2ST1 | TARS |
| 128 | TANC1 | CALM1 | ASCC3 | HMGB1 | RBPMS | TOP2A |
| 129 | KAZN | RPS29 | KIAA1217 | GJA1 | DLGAP1 | HSP90B1 |
| 130 | GPHN | H3F3B | MIR924HG | PODXL | PLEKHA5 | NASP |
| 131 | TAGLN | ATP5B | CRIM1 | TPI1 | IGF1R | RPS29 |
| 132 | ZFAND3 | FTL | PSD3 | CALM2 | PCCA | TPI1 |
| 133 | PTK2 | MDH1 | MACROD2 | RPL38 | FRMD4A | CCT3 |
| 134 | COMMD10 | ACTG1 | ATRNL1 | CYTB | ANKS1B | SSB |
| 135 | NRXN3 | AASS | FBXL17 | TPM3 | UTY | HNRNPK |
| 136 | FAM13A | G3BP2 | FER | SSB | FBXW7 | PGRMC1 |
| 137 | ITGAV | NACA | CADM2 | HNRNPK | MID1 | UBA52 |
| 138 | MAGI3 | TRIM71 | ADGRL3 | ATP6 | TTC28 | HMGB1 |
| 139 | PLCB1 | EPCAM | DLGAP1 | PGRMC1 | PTK2 | RPS28 |
| 140 | TEAD1 | ND5 | RSRC1 | TARS | TTN | EEF1B2 |
| 141 | TBCK | ND4L | RFWD2 | ATP5A1 | LOC101929378 | HMGB2 |
| 142 | METTL15 | CCT3 | IGF1R | COX7C | ROBO1 | KPNA2 |
| 143 | FRYL | SSB | ADGRV1 | HMGB2 | RAPGEF2 | RPL27 |
| 144 | ASCC3 | AP1S2 | RFX7 | ND4 | LRRC4C | YWHAB |
| 145 | ZNF804A | TDGF1 | CNTN4 | PPIA | RORA | IPO5 |
| 146 | PLXNA2 | CYTB | LDLRAD3 | ND2 | ZSWIM6 | YBX1 |
| 147 | LTBP1 | LRRN1 | FTO | ATP8 | RAB28 | CALR |
| 148 | IGF2BP2 | ITGA6 | DOCK3 | YWHAZ | TENM4 | PTGES3 |
| 149 | EXOC4 | TPI1 | NRXN1 | EEF1B2 | ADAMTS19 | CCT6A |
| 150 | ANK2 | ATP5A1 | FARS2 | HSPA4 | MEF2A | TPM3 |
| 151 | ANXA2 | RPS21 | KLF8 | COX2 | EXOC4 | PSMA4 |
| 152 | PICALM | TUBA1B | LCORL | CCT3 | TCF7L2 | PDIA3 |
| 153 | LRP1B | GJA1 | FMNL2 | CALR | SIK3 | SRSF3 |
| 154 | AGAP1 | PAICS | PAM | HNRNPU | LINC01572 | CCT5 |
| 155 | SPATS2L | PPIA | MGAT5 | ND6 | TASP1 | CFL1 |
| 156 | RAPGEF2 | SRSF3 | PCCA | DPPA4 | RPS6KA2 | SMARCA5 |
| 157 | INPP4B | RPS28 | MAGI1 | YWHAB | TBC1D22A | PLS3 |
| 158 | ERBB4 | PRDX1 | STK3 | HIST1H1D | PTPRT | XRCC6 |
| 159 | NEK7 | ND2 | CHODL | RPL27 | NEAT1 | TMSB10 |
| 160 | PCAT14 | SUPT16H | ARHGAP42 | ND1 | CAMKMT | PSMA7 |
| 161 | AUTS2 | ND6 | COG5 | CFL1 | FARS2 | ND6 |
| 162 | SGCD | TOP2A | KIAA0825 | PDIA3 | MPPED2 | PAICS |
| 163 | PARD3 | ENO1 | ADK | TOP2A | ASCC3 | HNRNPU |
| 164 | LOC105377862 | G3BP1 | ULK4 | XRCC6 | STK3 | SOX2 |
| 165 | PKIB | SMARCA5 | CERS6 | RPS21 | UBE3D | CEBPZ |
| 166 | FAM155A | HIST1H1D | CAMKMT | RPS28 | MAPK10 | CSDE1 |
| 167 | PKNOX2 | MATR3 | PARP8 | ND4L | NRXN1 | DYNLL1 |
| 168 | COL4A2 | PTGES3 | LARGE | PTGES3 | GRID2 | DDX5 |
| 169 | SERINC5 | COX1 | PRTG | PAICS | CTNNA2 | COX7C |
| 170 | MEF2A | HIST1H1B | PLCB1 | HNRNPA3 | ATRNL1 | TCP1 |
| 171 | DIP2C | GSTP1 | GRID2 | TMSB10 | CDK8 | PEBP1 |
| 172 | RFWD2 | CLU | TMEM132B | IPO5 | PSPC1 | HMGB3 |
| 173 | ROBO1 | PWARSN | TRPS1 | UBA52 | MGAT5 | UCHL1 |
| 174 | LOC102467213 | HMGB1 | HS3ST4 | USP9X | FRAS1 | NORAD |
| 175 | CDKAL1 | ND1 | SBF2 | CEBPZ | ARID1B | DPPA4 |
| 176 | PRIM2 | EEF1B2 | ADAMTS12 | MDH1 | ARHGAP26 | CCT8 |
| 177 | BMPR2 | YBX1 | MAML3 | CCT6A | TMTC1 | RPS21 |
| 178 | ITPR2 | PFN1 | TLE4 | CCT5 | WWOX | RPL38 |
| 179 | GSK3B | DEK | UBE3D | SRSF3 | TRPS1 | CLDN6 |
| 180 | SCAPER | UCHL1 | FIGN | SUPT16H | JAZF1 | SUPT16H |
| 181 | SUPT3H | PDIA3 | PCNX2 | EPCAM | CADM2 | CHD4 |
| 182 | SPATA5 | FGFR1 | PKP4 | CLDN6 | NLK | BEX3 |
| 183 | LHFP | COX2 | NSMCE2 | DNMT3B | AKT3 | BTF3 |
| 184 | CASK | HNRNPK | DTNA | HMGB3 | SPIDR | CENPF |
| 185 | UTRN | HNRNPA3 | CACNA2D3 | DYNLL1 | ADGRA3 | SUMO2 |
| 186 | STK3 | ND4 | GNAQ | UCHL1 | CEP128 | EPCAM |
| 187 | TMEM135 | RPL38 | FOXP1 | PLS3 | COBL | MDK |
| 188 | MALAT1 | RPL27 | B3GALT1 | SLC16A1 | LMCD1-AS1 | DEK |
| 189 | KLF8 | ATP8 | UTY | BEX3 | CHODL | TDGF1 |
| 190 | GNAQ | MCM4 | LRRC4C | DEK | NBEA | MATR3 |
| 191 | ST6GALNAC5 | XRCC6 | TMEM135 | MFGE8 | EPC2 | G3BP1 |
| 192 | RORA | TMSB4X | STK33 | KPNA2 | PAN3 | ATP8 |
| 193 | DNAH14 | EIF4B | DNAH14 | SMARCA5 | ERBIN | LECT1 |
| 194 | BBS9 | KPNA2 | PCDH7 | UGP2 | FRYL | RSL1D1 |
| 195 | LCORL | FTH1 | AKAP13 | DDX5 | LINC01021 | KPNB1 |
| 196 | WWOX | PPP1CC | ARHGAP32 | CHD4 | LRBA | TKT |
| 197 | RNF217 | CALM2 | PPM1L | TDGF1 | CERS6 | CLTC |
| 198 | PTPN14 | YWHAE | COL4A5 | MCM4 | KANSL1 | RHOA |
| 199 | RSRC1 | CCT6A | MMP16 | STMN1 | DIAPH2 | COX3 |
| 200 | ANO6 | CDH1 | FRYL | NONO | MAML3 | ND2 |
| 201 | LOC339862 | UBB | RBMS1 | HNRNPM | PAM | ND4L |
| 202 | ITFG1 | HSPA5 | ZSWIM6 | BTF3 | EGFEM1P | UGP2 |
| 203 | NBAS | MSH6 | EDA | SRP14 | FBN2 | NDUFS5 |
| 204 | TENM4 | AKIRIN1 | DAB1 | TKT | AKAP13 | HSPA9 |
| 205 | LOC728755 | PEBP1 | MEF2A | NDUFS5 | ADK | PSIP1 |
| 206 | INVS | YWHAB | ASTN2 | PEBP1 | GNAQ | CBX3 |
| 207 | VTI1A | TMX1 | TEAD1 | PSIP1 | PDE4D | NONO |
| 208 | WLS | CALR | DPH6 | CSDE1 | FBXL17 | UBC |
| 209 | CEP128 | SEPHS1 | ITFG1 | CBX3 | MNAT1 | ND5 |
| 210 | AKAP13 | EIF3A | RAPGEF2 | CLU | CCNY | CCT2 |
| 211 | CERS6 | RCC2 | ELP4 | G3BP1 | MAGI2 | PODXL |
| 212 | FARS2 | LARP7 | CBLB | HSPA9 | PKP4 | HSPH1 |
| 213 | SH3RF1 | TPM3 | SLC25A21 | SUMO2 | SSBP2 | PDIA6 |
| 214 | GRIP1 | HSPA9 | PHLPP1 | MATR3 | CACNA2D1 | CCT4 |
| 215 | AFAP1 | XRCC5 | RBFOX1 | EIF5A | ZNF827 | ARL6IP1 |
| 216 | FOCAD | QSER1 | RAB28 | MORF4L1 | INTS6 | USP9X |
| 217 | ALCAM | KCNG3 | PHACTR1 | HMGN1 | TRPM3 | FKBP3 |
| 218 | FAT3 | SUMO2 | TASP1 | TERF1 | LDB2 | MFGE8 |
| 219 | SEMA3A | UBA52 | KDM4C | PSMA4 | TMEM108 | VCP |
| 220 | NSMCE2 | NONO | PDE4D | RPS26 | FGF13 | CNBP |
| 221 | CAP2 | HMGB2 | HS2ST1 | CCT4 | RALGAPA2 | MORF4L1 |
| 222 | RAD51B | ADAR | RBPMS | RSL1D1 | HS3ST4 | CYP51A1 |
| 223 | BCKDHB | HMGB3 | GTDC1 | USP44 | SLC25A21 | SLC16A1 |
| 224 | TULP4 | HELLS | TTC17 | TCP1 | FRMD5 | SRP14 |
| 225 | DPH6 | HSP90B1 | APP | PDIA6 | NHSL1 | PKM |
| 226 | FMNL2 | KPNB1 | NBAS | PSMA7 | TDRP | COX411 |
| 227 | ARHGAP10 | TKT | TBC1D22A | EIF4B | ZCCHC7 | EIF4B |
| 228 | TENM2 | NAP1L1 | FGF13 | KPNB1 | PTBP2 | CYTB |
| 229 | LOC101929378 | SALL2 | PTPRM | MDK | TRMT11 | SF3B1 |
| 230 | PCDH11Y | PWAR5 | SEMA3A | CCT2 | RFWD2 | COX1 |
| 231 | HMCN1 | CCT5 | TOX | IDH1 | TTC17 | HNRNPM |
| 232 | ZNF407 | SCD | FRAS1 | PKM | ELAVL2 | C11orf58 |
| 233 | ATRNL1 | C21orf59 | DENND1A | UBC | DIP2C | NAP1L1 |
| 234 | PDE10A | BPTF | SEMA6A | NORAD | XYLT1 | DNMT3B |
| 235 | TIAM1 | NDUFS5 | LOC107986770 | LRRN1 | SLC2A13 | USP44 |
| 236 | HS6ST2 | CSE1L | JAZF1 | ANP32E | CMIP | XRCC5 |
| 237 | SDC2 | CNBP | LOC107985675 | NOLC1 | ULK4 | IGF2BP1 |
| 238 | ANXA1 | SNHG5 | TMTC1 | MSH6 | UBE2E1 | ND1 |
| 239 | KALRN | SYN3 | ANK3 | RPS25 | SBF2 | PA2G4 |
| 240 | ARHGAP28 | MYL6 | PPARGC1A | HIST1H1E | TLAM1 | EIF3A |
| 241 | ZNF423 | CCT4 | RPS6KA2 | CNBP | RFC3 | ATP6 |
| 242 | LDLRAD3 | IDO1 | SGMS1 | IGF2BP1 | LOC107986777 | C14orf166 |
| 243 | PSD3 | RTN3 | LOC339862 | VCP | LOC105377134 | AZIN1 |
| 244 | RFX3 | ZNF770 | DIAPH2 | NAP1L1 | CDH4 | CCNG1 |
| 245 | NT5C2 | TRIM28 | CCDC91 | SLIRP | SEMA3A | STMN1 |
| 246 | WDFY3 | POLR3G | MNAT1 | ARL6IP1 | CNTN4 | NARS |
| 247 | FRMD4A | RPS26 | VWA8 | DHCR24 | DAPK1 | HMGCS1 |
| 248 | CDH13 | EIF4G2 | UNC5D | UQCRH | SEMA5A | RAD21 |
| 249 | PARD3B | CLIC4 | LOC100288637 | PA2G4 | ARHGAP32 | PARK7 |
| 250 | ARHGAP42 | SALL4 | EDIL3 | LECT1 | SPRED1 | ANP32E |
| 251 | COL4A5 | RSL1D1 | PTBP2 | HIST1H1C | RSRC1 | SKP1 |
| 252 | PIBF1 | CHD4 | USP25 | G3BP2 | PARD3B | NOLC1 |
| 253 | PAM | ANP32E | RNGTT | EIF3A | PRDM5 | COX2 |
| 254 | LOC105377901 | CENPF | PRKD1 | FKBP3 | ADAMTS12 | HNRNPDL |
| 255 | EXOC6B | PRR14L | MMS22L | LITAF | RGS7 | IDH1 |
| 256 | ZNF521 | DDX5 | FAM13A | RPL22 | PTPRM | PPP1CC |
| 257 | TMCC1 | YWHAZ | APBB2 | PARK7 | UTRN | MCM4 |
| 258 | ALPK2 | ZFP42 | DOCK1 | CLTC | BMPR1A | IQGAP1 |
| 259 | VWA8 | SPTBN1 | BCAS3 | AKIRIN1 | DYRK1A | STIP1 |
| 260 | RAB28 | NOLC1 | EHBP1 | CCT8 | WWC2 | ADH5 |
| 261 | CCSER2 | FKBP3 | WDR70 | DPYSL3 | KCTD8 | SPTBN1 |
| 262 | DANT2 | SRRM2 | LOC105377862 | CENPF | KLF8 | LITAF |
| 263 | RFX7 | PSMA4 | PTCHD1-AS | CYCS | MAPK8 | CBX1 |
| 264 | DDAH1 | PTPRZ1 | NCOA2 | SF3B1 | KCNQ1OT1 | MKI67 |
| 265 | ADAMTS6 | CCT2 | NTM | DBI | SGMS1 | DPYSL3 |
| 266 | NFAT5 | CKB | TLAM1 | C11orf58 | TMEM135 | RPS25 |
| 267 | COG5 | RIF1 | ADGRL2 | AZIN1 | APBB2 | RPL22 |
| 268 | KIRREL | UQCRH | TMEM108 | PPP1CC | TNS3 | YWHAQ |
| 269 | TASP1 | IGF2BP1 | MAPK8 | RHOA | UBE2E3 | EID1 |
| 270 | SPAG16 | CCNG1 | CLASP1 | XRCC5 | CHST9 | NDUFA4 |
| 271 | MYOF | RPS25 | RBMS3 | RCC2 | MACROD2 | G3BP2 |
| 272 | NLGN4Y | SF3B1 | TBL1XR1 | HSPH1 | FGF12 | RPS26 |
| 273 | LRFN5 | ILF3 | UTRN | HNRNPAB | CBLB | EPRS |
| 274 | CDC42BPA | ND3 | MAPK10 | OAZ1 | SH3RF1 | DLGAP5 |
| 275 | LINC01572 | HSPH1 | CCNY | SKP1 | PRKD1 | SSRP1 |
| 276 | COL4A1 | NORAD | TRAPPC9 | HNRNPDL | TBCK | PRDX6 |
| 277 | ABHD12B | HNRNPM | WDFY3 | EID1 | KDM4C | TERF1 |
| 278 | WDR70 | EPRS | NKAIN3 | ADAR | NCOA2 | CSE1L |
| 279 | STXBP5 | BEX3 | FBXW7 | APELA | NFIB | TPM4 |
| 280 | UST | AZIN1 | DANT2 | ALDOA | FOXN3 | UQCRH |
| 281 | CLASP1 | SSRP1 | WDR27 | COX411 | AGPAT4 | SRSF1 |
| 282 | HS6ST3 | MCM3 | MCC | SRRM2 | FER | MSH6 |
| 283 | SLC4A7 | TMEM167A | LTBP1 | DNAJA1 | EDA | CYCS |
| 284 | KCNT2 | BUB1 | BBX | MKI67 | CLASP1 | CLU |
| 285 | SFMBT2 | MORF4L1 | RFC3 | DDX6 | FANCL | ALDOA |
| 286 | LIMS1 | SRSF1 | NEDD4L | UQCRB | FAM160A1 | DDX21 |
| 287 | PTCHD1-AS | DDX21 | FARP1 | RAD21 | RMST | ATP50 |
| 288 | RANBP17 | RPL22 | ARHGAP26 | BSG | RAD51B | GNAS |
| 289 | SOX6 | ZFAND5 | PTPRT | SPTBN1 | KCNH8 | ZNF770 |
| 290 | FARP1 | LARS | ADGRA3 | YWHAQ | MIPOL1 | EIF5 |
| 291 | ERBIN | MED14 | MAGI3 | SNHG5 | PKN2 | LRRN1 |
| 292 | ROCK2 | RAD21 | LRRTM4 | NARS | XKR6 | CD63 |
| 293 | SRPK2 | ATP5H | GSK3B | CSE1L | PHACTR1 | RPL23A |
| 294 | TCF4 | MLEC | RGS7 | SON | GLCCI1 | GDI2 |
| 295 | IGF1R | HNRNPDL | DIP2C | RPL23A | DANT2 | CCNB1 |
| 296 | ZNF704 | SNTG2 | GLI3 | IQGAP1 | CADM1 | HDAC2 |
| 297 | LINC00458 | IDH1 | ZNF423 | DDX21 | ZNF423 | CLIC4 |
| 298 | PPFIBP1 | TRIM24 | PTPRN2 | HMGCS1 | ATXN7L1 | PTBP1 |
| 299 | MCC | CSDE1 | RALGAPA2 | TMBIM6 | STAG1 | OAZ1 |
| 300 | CHST9 | BTF3 | ST6GALNAC5 | NDUFA4 | PTEN | ADAR |
| 301 | PRKD1 | ARL6IP1 | SND1 | EPRS | SEMA6A | DDX6 |
| 302 | CNTN4 | PA2G4 | FTX | STIP1 | ETV6 | ACTR2 |
| 303 | PRICKLE2 | SMC3 | FRMD4A | ILF3 | MARK1 | EIF5A |
| 304 | CCNY | RPL23A | RERE | SSRP1 | EXOC6B | ZFP42 |
| 305 | DST | DDX6 | AIG1 | CBX1 | APP | DBI |
| 306 | AFF3 | PRDM14 | SCLT1 | CLIC4 | RNGTT | TOMM20 |
| 307 | NEBL | CCT8 | FANCL | HIST2H2AC | DPP6 | DNAJA1 |
| 308 | MNAT1 | CXADR | ZNF521 | SRSF1 | SLC4A7 | TMBIM6 |
| 309 | CTDSPL | HNRNPH3 | C15orf41 | ATP5H | DNAH14 | EIF5B |
| 310 | SYNE1 | HNRNPD | ATP9B | C14orf166 | PRICKLE2 | HMGN1 |
| 311 | PDE3B | TCP1 | NHSL1 | APEX1 | MED12L | C21orf59 |
| 312 | AMOTL1 | DYNLL1 | PAN3 | ITGA6 | SND1 | HNRNPH3 |
| 313 | KDM4C | HIST1H1E | TBC1D32 | HMGN2 | RBMS1 | BSG |
| 314 | NF1 | GDI2 | ALCAM | LARP7 | LRRTM4 | APELA |
| 315 | RIC1 | TARDBP | PALLD | ATP50 | PLCB1 | MRFAP1 |
| 316 | CEP112 | HNRNPAB | MED12L | SNRPD2 | TRAPPC9 | RCC2 |
| 317 | DOCK1 | MTHFD2 | PKN2 | MARCKSL1 | AIG1 | EDF1 |
| 318 | LRCH1 | XPO1 | PPP1R9A | FBL | C15orf41 | SRRM2 |
| 319 | RNGTT | TOMM20 | CDH4 | TRIM28 | MIR924HG | HIST1H1B |
| 320 | HSD17B12 | NUDT21 | STAG1 | EIF5B | FYN | ACLY |
| 321 | MIPOL1 | PRKDC | PCSK5 | PTBP1 | ATP9B | SLC25A3 |
| 322 | ATXN1 | DLGAP5 | PLD5 | HNRNPH3 | GTDC1 | ST13 |
| 323 | MB21D2 | OTX2 | BMPR1A | CD63 | DMTF1 | ILF3 |
| 324 | HPGD | IQGAP1 | SRPK2 | HIST1H2AM | LARP4B | EIF4G1 |
| 325 | TANC2 | CBX3 | ZNF407 | CYP51A1 | KDM6A | HINT1 |
| 326 | SCLT1 | PSMA7 | ERBIN | CCNG1 | BACH2 | ND4 |
| 327 | FOXO3 | HDAC2 | R3HDM1 | ZNF770 | UBAC2 | DHCR24 |
| 328 | LOC102724419 | COX411 | SHISA9 | ZFP42 | PLCB4 | FLNA |
| 329 | MACROD2 | YWHAG | ITPR2 | ND3 | DLG1 | SNRNP200 |
| 330 | PDLIM5 | VCAN | LMCD1-AS1 | ANP32A | RBM47 | RPL36AL |
| 331 | UBE2E3 | NDUFA4 | LOC105377134 | TARDBP | MAP2K4 | DDX3X |
| 332 | UNC5C | FAM60A | LOC102467213 | YWHAG | STK33 | TMX1 |
| 333 | LRRC4C | NARS | HS6ST3 | EIF5 | BBX | PRRC2C |
| 334 | MAPK8 | C14orf166 | TNS3 | AP2M1 | FARP1 | IGF2R |
| 335 | CUX1 | BSG | XKR6 | PSAP | ADGRL2 | APEX1 |
| 336 | EGFEM1P | OAZ1 | DLG1 | SLC25A3 | ITFG1 | PDIA4 |
| 337 | LOC105377134 | SLC25A3 | DPP6 | SERF2 | SPATS2L | SF3B2 |
| 338 | CHST11 | IMPDH2 | GPC3 | CCNB1 | ZNRF3 | ANP32A |
| 339 | PDZD2 | SYNCRIP | PIBF1 | C21orf59 | THSD4 | TRIM28 |
| 340 | MBNL1 | VCP | SAMD4A | ACLY | EHBP1 | UQCRB |
| 341 | TBC1D32 | PTBP1 | NEAT1 | EIF1 | HS6ST3 | PSMA2 |
| 342 | BCAS3 | CYCS | CDC42BPA | MRFAP1 | WDFY3 | PSAP |
| 343 | SPRED1 | CLTC | PRICKLE2 | RPL36AL | LOC101927668 | FAM136A |
| 344 | LOC100420587 | PARK7 | DST | GDI2 | C9orf3 | CCT7 |
| 345 | PKN2 | MARCKSL1 | CHST9 | MLEC | PTPRN2 | EIF1 |
| 346 | FBXW7 | PMAIP1 | UBE2E3 | HDAC2 | ELMO1 | SMC3 |
| 347 | IL17RD | EIF4A2 | ATXN7L1 | DDX3X | CHST11 | TMED2 |
| 348 | RAP1GDS1 | STIP1 | FAAH2 | EDF1 | LRRC8D | TAF7 |
| 349 | DCC | DUT | ELMO1 | COX5B | ATG10 | TPR |
| 350 | ADGRL2 | C1orf21 | PHF14 | EIF4G1 | MCC | MORF4L2 |
| 351 | LAMA2 | FAM136A | CLASP2 | APOE | DTNA | DDX18 |
| 352 | MACF1 | MCM6 | KCNQ1OT1 | TPM4 | MSI2 | FBL |
| 353 | LOC107986770 | ZNF217 | SIPA1L1 | TOMM20 | GSK3B | EIF4A2 |
| 354 | SCFD2 | CENPU | GREB1L | SEPHS1 | FTO | ATP5H |
| 355 | SNX9 | NOL11 | MAP4K3 | ATPIF1 | R3HDM1 | AKIRIN1 |
| 356 | ZNF827 | EID1 | KCTD8 | PRDX6 | CDYL | DSTN |
| 357 | EDIL3 | THRAP3 | WDPCP | ADH5 | FTX | HNRNPD |
| 358 | UTY | PPM1G | ZNF827 | GNAS | UVRAG | THRAP3 |
| 359 | LOC100288637 | NIPSNAP1 | STXBP5 | HIST1H3B | LOC100133091 | GLO1 |
| 360 | DOK6 | C11orf58 | TRMT11 | TMED2 | VTI1A | SON |
| 361 | SBF2 | IGF2BP3 | ZRANB3 | ILF2 | PRTG | TXN |
| 362 | PARVA | EIF5A | CADPS | ST13 | GLI3 | BZW1 |
| 363 | ZNF385D | APEX1 | MARK1 | CKB | INVS | AP2M1 |
| 364 | DLG1 | FKBP4 | TMCC1 | SF3B2 | ARHGAP42 | CNN3 |
| 365 | GRIK2 | DNAJA1 | MARCH1 | MORF4L2 | FIRRE | IMPDH2 |
| 366 | MAN1A2 | FBL | MLLT10 | CDH1 | MEMO1 | HN1L |
| 367 | ZEB1 | PDPN | TCF7L2 | IMPDH2 | PLD5 | RANBP2 |
| 368 | C15orf41 | ALDOA | STXBP5L | NFE2L1 | MMS22L | USO1 |
| 369 | HDAC8 | SNRPD2 | ASIC2 | ATF4 | ANAPC10 | SYNCRIP |
| 370 | TMEFF2 | PDIA6 | AGPAT4 | THRAP3 | HDAC9 | HNRNPR |
| 371 | CTNNA3 | NUFIP2 | C9orf3 | FLNA | NEK7 | ILF2 |
| 372 | BMPER | FUS | DDX10 | TMEM167A | LOC100288637 | TARDBP |
| 373 | MGAT5 | APOE | CDH2 | PRRC2C | RAPGEF6 | TPX2 |
| 374 | PCSK5 | PRRC2C | PRDM5 | HNRNPD | DPP10 | ZNF217 |
| 375 | TMEM232 | EIF2S3 | LOC729732 | USO1 | FAM135A | YWHAG |
| 376 | MLLT3 | ACVR2B | ZCCHC7 | SMC3 | SHISA9 | CACYBP |
| 377 | TBL1XR1 | SRP14 | CUX1 | FAM136A | VPS54 | PCBP1 |
| 378 | KLF7 | EIF5 | EPC2 | HINT1 | TBL1XR1 | PRKDC |
| 379 | RPS6KA2 | ATP50 | MSI2 | NUDT21 | ASTN2 | HNRNPH1 |
| 380 | LRRTM4 | DDX3Y | OSBPL10 | ZNF217 | WDR27 | PRPF8 |
| 381 | SLIT3 | MRFAP1 | WWC2 | ZFAND5 | TLE1 | SLIRP |
| 382 | RFC3 | RHOA | CHST11 | SALLA | MYO1D | DBN1 |
| 383 | HS3ST3A1 | SLIRP | SHROOM3 | MCM3 | FOXJ3 | STRAP |
| 384 | TBC1D22A | CCNB1 | CADM1 | CACYBP | CADPS | CKB |
| 385 | ATP9B | UQCRB | CMIP | NDUFA13 | NKAIN3 | CAPRIN1 |
| 386 | SPATA6 | SERF2 | ZBTB20 | HN1L | EDIL3 | PSMA3 |
| 387 | OSBPL8 | TUG1 | CSMD2 | EIF4A2 | LOC101929194 | SNRPD2 |
| 388 | ARHGAP32 | NAA50 | RMST | PSMA2 | STXBP5 | ITGA6 |
| 389 | KMT2C | YWHAQ | MPPED2 | KHSRP | ASIC2 | SERF2 |
| 390 | GLS | CTSC | BAZ2B | ATP5G3 | VWA8 | COX5B |
| 391 | ZNF608 | ADH5 | BICD1 | RPL41 | LOC105378031 | FADS1 |
| 392 | LOC107985962 | SF3B2 | ZMAT4 | PCBP1 | CACHD1 | CCND1 |
| 393 | PTCHD4 | SNRNP200 | HCN1 | CCT7 | MB21D2 | CTNNB1 |
| 394 | LOC102724210 | DHCR24 | DAPK1 | ACTR2 | PRR16 | RBMX |
| 395 | ZNF516 | EIF5B | CASK | DDX18 | ZNF385D | MLEC |
| 396 | FYN | NFYB | ZC4H2 | PPM1G | FBXO11 | TMEM167A |
| 397 | DOCK7 | SON | DYRK1A | PSMD2 | ST6GALNAC5 | FUS |
| 398 | GLIS3 | PCNA | MGAT4C | SNRNP200 | OSBPL10 | HDLBP |
| 399 | COPG2 | RPL36AL | JMJD1C | PDIA4 | E2F3 | EIF3L |
| 400 | ASTN2 | RAB13 | LOC100133091 | TMX1 | PDSS2 | METAP2 |
| 401 | DLC1 | MGEA5 | INVS | DSTN | ADGRB3 | SEPHS1 |
| 402 | PLEKHA5 | ZNF483 | CADPS2 | SNRPD3 | LINC01619 | LARS |
| 403 | COL1A2 | LRRC75A-AS1 | MTHFD1L | PRMT1 | KCNQ3 | SKIL |
| 404 | YAP1 | FLT1 | SCAPER | SKIL | RBM33 | DDX17 |
| 405 | LRIG3 | ZIC3 | PRKCA | DBN1 | WDR70 | TOP2B |
| 406 | PCNX4 | SLC6A8 | CDK8 | GLO1 | UBE3C | HNRNPAB |
| 407 | NEDD9 | ATF4 | UBAC2 | FKBP4 | HDAC4 | MAPRE1 |
| 408 | ZNF385B | HIST1H3D | SH3RF1 | RBMX | MAST4 | SRP72 |
| 409 | KRT8 | NDUFA13 | KIAA1328 | DUT | GABRB3 | PRMT1 |
| 410 | LOC105377860 | STRAP | CAMK2D | SERPINB9 | STXBP5L | ATP5G3 |
| 411 | DLG2 | CFL1 | PEAK1 | STRAP | ABHD17B | NUDC |
| 412 | AGPAT4 | PCBP1 | ARHGEF10 | TAF7 | LOC100288798 | CAND1 |
| 413 | GRIA1 | PSMA2 | FAM135A | CNN2 | FAM168A | DDX1 |
| 414 | ADK | SEMA3E | CNTLN | LRRC75A-AS1 | PEAK1 | ND3 |
| 415 | ITGB8 | IPW | HDAC8 | TFAM | PCNX1 | NUDT21 |
| 416 | FAM172A | DDX17 | KDM6A | SALL2 | ROR1 | CALU |
| 417 | STAG1 | IPO7 | MBNL1 | ERH | NR2C2 | NIPSNAP1 |
| 418 | ETV6 | TAF7 | SNTG2 | HNRNPR | KIAA1324L | BUB1 |
| 419 | VPS54 | SF3A3 | ATXN1 | HDGF | CCDC91 | NOL11 |
| 420 | ZBTB20 | BNC2 | BRAF | DNTTIP2 | NRF1 | ETF1 |
| 421 | LOC107987087 | TFAM | RTTN | TXN | NOVA1 | IPO7 |
| 422 | RERE | DHX9 | NAV2 | NUDC | USP6NL | SERPINH1 |
| 423 | MMP16 | COX5B | MAN2A1 | SSBP1 | SNTB1 | SF3A3 |
| 424 | LOC105378031 | HNRNPH1 | LRRC8D | METAP2 | PPP2R2B | CAP1 |
| 425 | TRAPPC9 | F11R | GLI2 | EIF2S2 | GPATCH2 | EIF2S2 |
| 426 | ULK4 | CYP2S1 | RAP1GDS1 | PRPF8 | ELP4 | MARCKSL1 |
| 427 | ATG10 | SERPINB9 | RAPGEF6 | DLGAP5 | LOC102723568 | SALL4 |
| 428 | NAALADL2 | HDGF | CHN1 | HNRNPL | PDE3B | ATF4 |
| 429 | GPATCH2 | PHF21B | ERC1 | TXNRD1 | ZNF519 | MARCKS |
| 430 | NKAIN3 | ANP32A | HDAC9 | HDLBP | SHANK2 | EIF3M |
| 431 | DNAJC15 | KHSRP | IGF2BP2 | HIST1H3D | NBAS | PSMD2 |
| 432 | KIAA1328 | USP7 | TTLL5 | PMAIP1 | THSD7A | ZFAND5 |
| 433 | FAAH2 | DDX3X | RPS6KC1 | HIST1H4E | SIPA1L1 | VIM |
| 434 | FGF12 | SNRPD3 | SHANK2 | MARCKS | TAF3 | EIF2S3 |
| 435 | TTC17 | CBX1 | COL11A1 | PRKDC | ANO10 | KDM5B |
| 436 | JAZF1 | METAP2 | ANO10 | DDX1 | MAGI3 | TXNRD1 |
| 437 | UBE2E1 | TMBIM6 | FAM160A1 | PWARSN | ERBB4 | PGK1 |
| 438 | PKP4 | RPL41 | DYM | PSMD7 | HCN1 | CDH1 |
| 439 | RASAL2 | EDF1 | SCFD2 | MTHFD2 | PPARGC1A | XPO1 |
| 440 | LOC107985710 | EIF3L | SYNDIG1 | ETF1 | RIC1 | CNN2 |
| 441 | S100A11 | NFE2L1 | CALD1 | MRPS34 | RNF13 | NDUFA13 |
| 442 | CHD7 | ETF1 | FOXN3 | RANBP2 | BRAF | FKBP4 |
| 443 | GNB4 | CACYBP | SAMD12 | HNRNPAO | PELI2 | KHSRP |
| 444 | KRT18 | SMARCC1 | FGF12 | COX7A2 | TBC1D32 | SMC4 |
| 445 | EVI5 | HN1L | UBE2E1 | EIF3L | JMJD1C | SALL2 |
| 446 | C9orf3 | SIRT1 | NEBL | NIPSNAP1 | SYN2 | SPTAN1 |
| 447 | TSPAN18 | CHD8 | SEMA5A | DNAJC8 | PLPP3 | SNRPD3 |
| 448 | KCNJ3 | RBMX | LOC101927668 | RAB13 | FNDC3A | SNHG5 |
| 449 | NHSL2 | PPM1B | TULP4 | PSMB1 | FIGN | SRP9 |
| 450 | ITPR1 | ST13 | ANKRD17 | LARS | KANK1 | SSR3 |
| 451 | MARCH1 | KDM5B | NLGN4X | PGK1 | PIP4K2A | PCNP |
| 452 | MAP4K3 | GNPTAB | CCSER2 | BZW1 | SAMD4A | HIST1H1D |
| 453 | LYPLAL1 | MRPS34 | NELL2 | SLC26A3 | ANK3 | HMGN2 |
| 454 | TNC | PAIP2 | PTPN4 | SUB1 | TMEM161B-AS1 | LARP7 |
| 455 | PHF14 | SKIL | COBL | MAPRE1 | WDFY2 | SQLE |
| 456 | ARID1B | SSBP1 | CTTNBP2 | PHB | FGD4 | HDGF |
| 457 | LINC01515 | DNTTIP2 | LOC100288798 | SYNCRIP | TMCC1 | MCM6 |
| 458 | FAM171A1 | KNOP1 | GABRB3 | CENPU | SCAF8 | ATP5C1 |
| 459 | LOC100133091 | ATPIF1 | THSD7A | HSBP1 | NELL2 | MTHFD2 |
| 460 | BBX | KIF1A | PDE3B | SSR3 | PIBF1 | HNRNPL |
| 461 | MED12L | ACLY | STAU2 | PAIP2 | ZNF407 | MYH10 |
| 462 | SSH2 | HNRNPC | BTBD9 | SNRPF | PTPN4 | DHX9 |
| 463 | HOOK3 | PRDX6 | FNDC3A | TRIM71 | NOS1AP | MCM3 |
| 464 | MPDZ | CDCA7L | COA1 | SF3A3 | MTHFD1L | DUT |
| 465 | BACH2 | NAP1L3 | DLEU1 | SERPINH1 | ZNF608 | FDFT1 |
| 466 | FLNC | PPAT | LINC01515 | HNRNPH1 | CAMK2D | PSMB1 |
| 467 | VIM | HNRNPL | MKL2 | FUS | GREB1L | HMGCR |
| 468 | ESRRG | PSMB4 | FRMD5 | ATP5J2 | CCSER2 | PPM1G |
| 469 | ACTN4 | ATP5G3 | POLA1 | PSMA3 | SLC44A5 | HYOU1 |
| 470 | PPM1L | DANCR | NLK | IPO7 | SRPK2 | SEC61B |
| 471 | LOC107985675 | CCT7 | PDZRN3 | MRPL51 | ITPR2 | XPOT |
| 472 | RTTN | VRTN | FYN | RANBP1 | COL25A1 | SFPQ |
| 473 | CSRNP3 | ZNF146 | NAV3 | SLC25A5 | FGF2 | DDX3Y |
| 474 | STIM2 | RAD50 | FAM172A | RPL13AP5 | NAV2 | DNTTIP2 |
| 475 | USP25 | HIST1H1C | SUMF1 | NOP56 | SHROOM3 | EIF3E |
| 476 | COLEC12 | PRDX2 | ROR1 | CALU | MLLT10 | DNAJC8 |
| 477 | DNAJC1 | ZFP36L2 | PICALM | HIST1H2AJ | DOCK3 | ACVR2B |
| 478 | TENM1 | PKM | KANSL1 | NOL11 | FAM169A | MRPS34 |
| 479 | NHS | SNRPF | PCNX1 | ABCF1 | BTBD9 | DCP2 |
| 480 | ANKRD6 | RABGAP1L | SPRED1 | HNRNPF | AUH | SLC26A3 |
| 481 | CREB5 | DBI | RBM26 | TMEM97 | DDX10 | CENPU |
| 482 | RBPMS | SRP9 | TMEM178B | MCM6 | KALRN | SLC38A2 |
| 483 | COL25A1 | PSAT1 | ATG10 | SOD1 | CTNNA3 | HNRNPAO |
| 484 | SPRED2 | HMGN1 | FIRRE | NUFIP2 | SCMH1 | CDC42 |
| 485 | C5orf42 | MIAT | BACH2 | SRP9 | ZRANB3 | SMARCE1 |
| 486 | CPNE8 | HNRNPF | SLC2A13 | PRR14L | ATG7 | GARS |
| 487 | HDAC9 | AP2M1 | CTNNA3 | ACVR2B | CALN1 | FXR1 |
| 488 | HS2ST1 | SNHG16 | LINC01122 | TCEB2 | EPC1 | TFAM |
| 489 | BCAT1 | RANBP2 | SIK3 | HYOU1 | CDK19 | PWARSN |
| 490 | MEIS2 | RBM27 | ADGRB3 | CNN3 | SIPA1L3 | RPL41 |
| 491 | MKLN1 | EIF1 | LOC105378031 | COX8A | MKLN1 | ABCF1 |
| 492 | MAP2K4 | TFRC | MIR325HG | EIF4EBP2 | NTM | LAPTM4A |
| 493 | UBE3D | LTA4H | TNKS | CCND1 | SYNDIG1 | PRR14L |
| 494 | AP3B1 | SNRPE | DYNC2H1 | CAP1 | SLC16A10 | CHD8 |
| 495 | CDH6 | PRMT1 | PELI2 | EIF2S3 | PCMTD1 | MRPL51 |
| 496 | DEC1 | MYH10 | GRB10 | SNHG16 | TSPAN5 | CCNI |
| 497 | BTBD9 | JARID2 | P3H2 | ENY2 | CAMTA1 | ID1 |
| 498 | SDK2 | XPOT | PSPC1 | PSMB4 | SNTG2 | ARCN1 |
| 499 | MIR99AHG | AHCY | FN1 | SNRPE | HERC4 | PSMD7 |
| 500 | TNIK | ERBB2 | HIVEP1 | SEMA3E | NR3C2 | LDHA |
| 501 | BMPR1A | EPB41 | NEK7 | TFRC | PHF21A | HADHA |
| 502 | TMEM131 | ANKRD18CP | GLCCI1 | UBTF | TRAF3IP2-AS1 | SLC25A5 |
| 503 | RBM47 | SRSF10 | WDFY2 | UQCR10 | ZNF277 | NAA50 |
| 504 | EPHA7 | SNRPD1 | LDAH | CKS2 | LOC339862 | USP7 |
| 505 | MBNL2 | CPSF2 | GPATCH2 | COX6B1 | RUNX1T1 | SOD1 |
| 506 | FUT8 | SRSF2 | PPP2R2B | LDHA | PALLD | NAE1 |
| 507 | FAM110B | NSD1 | PCMTD1 | IGF2R | KIAA1328 | ANP32B |
| 508 | MDGA2 | PRPF8 | SUCLG2 | CAPRIN1 | RIMS1 | SUB1 |
| 509 | SCMH1 | TCOF1 | THSD4 | ACAA2 | SERINC5 | SRRM1 |
| 510 | SMAD3 | LARS | BRE | OTX2 | ATXN1 | COPB2 |
| 511 | SLC2A13 | PSAP | CHD6 | SMARCE1 | LOC105377329 | ATPIF1 |
| 512 | VAV3 | SKP1 | ACOXL | TPR | BAZ2B | PAPOLA |
| 513 | NEDD4L | DBN1 | SPATS2L | KNOP1 | DAB1 | REST |
| 514 | MBOAT2 | SFPQ | CDH11 | GARS | FAM189A1 | SSBP1 |
| 515 | RPS6KC1 | EIF2S2 | CHD2 | HSPE1 | 1-Mar | ZNF146 |
| 516 | KHDRBS3 | NDUFV2 | SYT14 | SNRPD1 | KANSL1L | MAP1B |
| 517 | SESTD1 | BCLAF1 | DMTF1 | NAA50 | CSTF3 | PRPF40A |
| 518 | LRRFIP1 | HIST1H1A | ST6GAL1 | SOLE | RABGAP1 | SERPINB9 |
| 519 | UBAC2 | RMST | DMD | FDFT1 | NXPH2 | PHB |
| 520 | NPSR1-AS1 | HNRNPAO | LOC102723568 | ATP5F1 | MAP4K3 | HNRNPF |
| 521 | GALNT13 | NOP56 | SLC24A2 | GPX4 | PBX1 | RANBP1 |
| 522 | TLN2 | MAP7 | ANAPC10 | ATP5L | ARHGEF10 | SUMO1 |
| 523 | KIAA1109 | ERH | ANO6 | GNL3 | TTLL5 | PAIP2 |
| 524 | WDR27 | GAS5 | IL1RAPL1 | KARS | STRN3 | RIF1 |
| 525 | FREM1 | NAE1 | ZNF608 | ATXN7L3B | FGFR2 | SEPT2 |
| 526 | NES | RPL13AP5 | XKR4 | PCNA | LINC01515 | BCLAF1 |
| 527 | GMDS | DDX18 | CAMTA1 | ZNF146 | MKL2 | ARPC2 |
| 528 | EFR3A | GNAS | AP3B1 | UBE2I | CSMD2 | COX7A2 |
| 529 | ACSS3 | GPR176 | LOC107983984 | CCNA2 | CDK13 | APLP2 |
| 530 | JMJD1C | EIF3E | SNTB1 | SEPW1 | PDE7A | ERH |
| 531 | SND1 | PSMA3 | MYCBP2 | C1QBP | DYM | ACAA2 |
| 532 | PPARGC1A | MGAT4C | KIAA1324L | EIF3M | ATP8A2 | ATP5F1 |
| 533 | CHD2 | PHB | SMAD2 | NFYB | RTTN | HSBP1 |
| 534 | MEST | AARS | COL25A1 | ID1 | ARHGAP44 | LMAN1 |
| 535 | CCDC91 | CCNA2 | PBX3 | PFDN5 | SCLT1 | DKC1 |
| 536 | RREB1 | HINT1 | ETV6 | SRP72 | RNF38 | CKAP5 |
| 537 | SLC8A1 | ACAA2 | SCMH1 | FADS1 | CHN1 | RPL13AP5 |
| 538 | CCND2 | NOP58 | TBL1X | GOT2 | WDPCP | ZC3H15 |
| 539 | RSU1 | SUMO1 | UVRAG | TCOF1 | COA1 | KIF11 |
| 540 | MIR325HG | ABCF1 | PLCH1 | TUBB2B | DISP1 | PSMD12 |
| 541 | ADAM19 | TPR | CEP83 | PAPOLA | CTBP2 | CLTA |
| 542 | SRGAP1 | ZNF281 | FLRT2 | RSL24D1 | CDC14A | MRPL3 |
| 543 | CACNA2D3 | SLC25A5 | RNF130 | PCNF | BRINP1 | EIF4EBP2 |
| 544 | SHISA6 | SLC26A3 | SPATA6 | PSMC5 | GAB1 | NREP |
| 545 | TRMT11 | GGCT | EIF4G3 | NAE1 | DLEU1 | HIF1A |
| 546 | CBFA2T2 | MAL2 | CSRNP3 | HMGCR | RBFOX1 | SMARCC1 |
| 547 | SDCCAG8 | PSMD7 | PTEN | DKC1 | PRKCA | RAB13 |
| 548 | LRBA | IGF2R | LRRC16A | F11R | CADPS2 | COX8A |
| 549 | DUSP6 | SCG3 | KCNQ3 | TMA7 | RAP1GDS1 | CCND2 |
| 550 | ZFPM2 | UBTF | NRIP1 | SEC61B | KSR1 | PGD |
| 551 | BMPR1B | NUDC | PATJ | CLTA | EPB41L4A | SRSF7 |
| 552 | NCOA2 | PAPOLA | NAALADL2 | YBX3 | TULP4 | ATP5J2 |
| 553 | ANKRD36 | ENAH | TSPAN5 | PSMD12 | CDC42BPA | FASN |
| 554 | TTLL5 | EIF3M | MKLN1 | SRSF7 | LOC107985675 | TRIM71 |
| 555 | BARD1 | CKS2 | CACNB2 | TUG1 | GRB10 | LRRC75A-AS1 |
| 556 | RUFY3 | COX8A | STRN3 | PSME3 | GLI2 | PTPN11 |
| 557 | CMIP | MORF4L2 | CHD9 | ATP5G2 | RRAS2 | SMS |
| 558 | EDNRB | DKC1 | DCAF6 | BCCIP | MGAT4C | PSMB4 |
| 559 | CLASP2 | PSMD2 | HSD17B12 | PGD | ZNF521 | CCNA2 |
| 560 | MYH9 | PCDH18 | MYO1D | TALDO1 | ZBTB20 | AP1S2 |
| 561 | DYRK1A | NRXN1 | HDAC4 | ARCN1 | FAAH2 | NFE2L1 |
| 562 | ANAPC10 | TALDO1 | KMT2C | ATP6V1G1 | DCAF6 | TALDO1 |
| 563 | MAMDC2 | GOT2 | SNX13 | EIF3I | CHRM3 | SRSF10 |
| 564 | ADGRA3 | EIF4G1 | ACBD6 | PDPN | KCNMA1 | TUG1 |
| 565 | USP47 | SLTM | NHSL2 | DDX17 | RBMS3 | TMEM97 |
| 566 | ID3 | MKI67 | MPDZ | HIST1H2AG | GRHL2 | CKAP2 |
| 567 | CDK8 | SRSF7 | XYLT1 | PABPC4 | ADARB2 | KNOP1 |
| 568 | ZNF148 | ATP5G2 | PUM2 | KDM5B | ZNF124 | ATXN7L3B |
| 569 | MYO9A | PSMB1 | HERC4 | SLC38A2 | RIC8B | RBM3 |
| 570 | STAU2 | MDN1 | NF1 | SUMO1 | SCFD2 | SNRPE |
| 571 | REV3L | ROR1 | ANO4 | CDC42 | GPC3 | SUGT1 |
| 572 | PVT1 | PGK1 | KCNMA1 | PRDX2 | CHD6 | UBE21 |
| 573 | STXBP5L | TUBB2B | ZNF385B | COPB2 | LOC100507053 | RAD50 |
| 574 | GAB2 | BCOR | CEP192 | ATP5C1 | PDZD2 | PCBP2 |
| 575 | MKL2 | SMPDL3B | NOVA1 | ZFP36L2 | PTCHD1-AS | ACAT2 |
| 576 | ZC4H2 | GARS | EPB41L2 | FASN | LTBP1 | HSPE1 |
| 577 | LOC105375334 | SLC38A2 | PDLIM5 | SRRM1 | ZC4H2 | SNRPF |
| 578 | PLXDC2 | FEM1B | CHCHD3 | SAP18 | NAV3 | CKS2 |
| 579 | ATG7 | PABPC4 | FUT8 | RIF1 | SORCS1 | TAX1BP1 |
| 580 | LIMCH1 | TSSC2 | PIP4K2A | NDUFS6 | FHIT | ZNF483 |
| 581 | NFIA | NREP | FGD4 | ATP5 | SASH1 | OSTC |
| 582 | CEP192 | WDHD1 | MAP2K4 | GGCT | LOC105374945 | ATP5L |
| 583 | KIAA1324L | UBA2 | SLC35F1 | MGEA5 | SMAD2 | TUBA1A |
| 584 | SIPA1L1 | YBX3 | KALRN | RCN2 | LHFP | SNRPD1 |
| 585 | NEO1 | AIF1L | ZNF385D | NOL7 | FN1 | ABCE1 |
| 586 | DENND1A | CNOT1 | FGGY | GSPT1 | PITPNC1 | PPIG |
| 587 | MMS22L | CAPRIN1 | ANKIB1 | BUB1 | KDM2A | CPSF2 |
| 588 | CASC15 | UBC | RIMS1 | SRSF2 | PARGP1 | GOT2 |
| 589 | ZNF124 | PLPP1 | CDYL | NDUFB10 | ACOXL | SNHG16 |
| 590 | FAM126A | ARFGEF3 | PDZD2 | ZC3H15 | TRABD2B | PPAT |
| 591 | C4orf51 | CHEK1 | LOC105377860 | DSG2 | MBOAT2 | NUFIP2 |
| 592 | SAMD3 | CBL | MEMO1 | PHB2 | ATF7IP2 | MCL1 |
| 593 | PARK2 | HIP1 | ACACA | TXNDC17 | CNTLN | PFDN5 |
| 594 | RUNX1T1 | DNAJC8 | SLC4A7 | APLP2 | LRRC16A | SEMA3E |
| 595 | DLEU1 | RBM12 | FAM168A | PSMB6 | MIR325HG | TCEB2 |
| 596 | PRSS23 | MMADHC | COL4A2 | RTN3 | ATAD2B | SNX6 |
| 597 | SULF1 | ILF2 | KIRREL | SNRPB | STXBP4 | ATP5G2 |
| 598 | LOC100506990 | PSMB6 | SHPRH | HELLS | SLAIN1 | YBX3 |
| 599 | NEXN | DDX1 | THRB | PDAP1 | BMPR2 | MGEA5 |
| 600 | UBE3C | PPIG | LOC107987083 | NDUFB9 | BRIP1 | ATP6V1G1 |
| 601 | DPP6 | FXR1 | GMDS-AS1 | DDX24 | TMEM178B | EIF3] |
| 602 | WWTR1 | ATP5L | ANKRD36 | PSMD4 | PDZRN3 | PRDX2 |
| 603 | LOC107986021 | NAA15 | CNOT2 | CPSF2 | RPS6KC1 | HELLS |
| 604 | ANKIB1 | SUGT1 | BNC2 | VAT1 | RICTOR | LARS |
| 605 | DYM | HIST2H2AC | LOC107986777 | COX5A | ST7 | GGCT |
| 606 | USP53 | RSL24D1 | PITPNC1 | DHX9 | PHF14 | KARS |
| 607 | HECW2 | RBM25 | ATP8A2 | DANCR | CSRNP3 | TMED10 |
| 608 | DDX10 | MRPL3 | RIC1 | CCDC47 | MBNL1 | RPL7L1 |
| 609 | PRDM5 | EIF4EBP2 | MBOAT2 | DDX3Y | SUMF1 | PSME3 |
| 610 | LOC100288798 | LRPPRC | SVIL | WBP11 | PRKY | COX6B1 |
| 611 | COL5A1 | DDX46 | LOC101928570 | PNRC2 | NEBL | PSMB6 |
| 612 | BRE | GPI | HACE1 | XPO1 | FOXO3 | PSMD4 |
| 613 | ARHGAP26 | MRPL51 | SSH2 | ZNF483 | NRIP1 | NUSAP1 |
| 614 | SLC20A2 | PRPF40A | VPS54 | ACP1 | LMBR1 | LAPTM4B |
| 615 | NHSL1 | DSG2 | PBX1 | POLR2L | BCL11A | ZIC3 |
| 616 | SOS1 | RARRES2 | NEO1 | LSM4 | NEO1 | PDPN |
| 617 | MTHFD1L | GLO1 | KIF13A | PAFAH1B2 | SLC20A2 | LSM4 |
| 618 | CNOT2 | PHGDH | FGF2 | TOP2B | SCN8A | RSL24D1 |
| 619 | KDM6A | ID1 | TLN2 | SHFM1 | AHCYL2 | TUBB2B |
| 620 | HHLA1 | MGST1 | LARP4B | CHCHD2 | DPH6 | MMADHC |
| 621 | PCMTD1 | GLUL | RAPGEF5 | PSMC3 | SAMD12 | RTN3 |
| 622 | PCDH10 | PAFAH1B2 | VPS13A | SPTAN1 | IGF2BP2 | UBTF |
| 623 | RIN2 | SOD1 | STK39 | MGST1 | LOC645513 | GFPT1 |
| 624 | RAPGEF6 | PFDN5 | CDK6 | RBM3 | LRCH1 | BCCIP |
| 625 | SASH1 | ATXN7L3B | PPM1H | SFPQ | CHD9 | DDX24 |
| 626 | SUCLG2 | TAF15 | RBM47 | PPAT | CBR4 | ATP5J |
| 627 | FNDC3A | WBSCR17 | PCLO | SOX4 | CUX1 | NES |
| 628 | OPCML | BUB3 | KIAA1958 | KDELR1 | HACE1 | PAFAH1B2 |
| 629 | PHLPP1 | EP300 | CDC73 | EIF3K | LRIG1 | GSPT1 |
| 630 | TSPAN5 | CADM2 | ARID2 | OSTC | UBE2W | DDX46 |
| 631 | MGMT | KIF11 | UBE3C | HIST1H2BH | REPS1 | UQCR10 |
| 632 | MEMO1 | EIF1B | TCF7L1 | SRSF10 | LOC729732 | HEATR1 |
| 633 | HERC4 | LINC00371 | MB21D2 | USP7 | CDH8 | NOP56 |
| 634 | RALGAPA2 | OAZ2 | WDR7 | LTA4H | PICALM | GOLGB1 |
| 635 | ADARB2 | BST2 | ERBB4 | NAP1L3 | CLASP2 | GPX4 |
| 636 | PTBP2 | COX6B1 | LMBR1 | USP1 | LINC-PINT | SEPW1 |
| 637 | FRMD6 | HNRNPR | DISP1 | NREP | SPPL3 | F11R |
| 638 | TPM1 | DCP2 | LYPLAL1 | TCEAL4 | DGKH | DENR |
| 639 | NCALD | NDUFB10 | BMPR2 | PPIG | MAP2K5 | MAGED2 |
| 640 | CCSER1 | AMD1 | AGTPBP1 | PRRC2A | SNX13 | ANXA5 |
| 641 | EPHA3 | LSM4 | SOS1 | VCAN | SLC35F3 | NFYB |
| 642 | RBM26 | RBPJ | CHRM3 | CCNI | MTMR3 | EIF4H |
| 643 | LOC105370504 | GNL3 | RICTOR | LSM3 | CDH13 | MGST1 |
| 644 | MAP4K5 | HMMR | QKI | PCBP2 | TCF7L1 | ARPC5 |
| 645 | ANTXR1 | LAPTM4B | AHCYL2 | EIF4H | SLC25A26 | CENPE |
| 646 | HMGA2 | STK26 | DTNB | TMEM14C | COL4A5 | PSMC5 |
| 647 | ITGB5 | RND2 | LOC101928096 | MMADHC | RPTOR | YME1L1 |
| 648 | ATXN7L1 | TFDP2 | BARD1 | PTTG1 | FNIP1 | TAF15 |
| 649 | MIR924HG | APRT | KCNH8 | TOMM6 | SLC24A3 | BPTF |
| 650 | CDH10 | PPT1 | HIBCH | SEPT2 | MRPS28 | TSPAN6 |
| 651 | CACNA1C | PSMD12 | EV15 | ZIC3 | SLX4IP | CHCHD2 |
| 652 | CADPS | GSPT1 | LGR4 | PPDPF | DIP2B | VCAN |
| 653 | RALYL | IRX2 | GALNT7 | HIST1H1A | AHI1 | PNRC2 |
| 654 | NPAS3 | TMEM97 | CALN1 | GMFB | TPST1 | PABPC4 |
| 655 | TAF3 | GPRC5B | CDK19 | XPOT | KIAA1468 | PEG10 |
| 656 | FAT4 | EIF3I | ATP2B1 | TIMM13 | STX18-AS1 | VAT1 |
| 657 | ADAMTS3 | PNRC2 | VAV3 | NDUFV2 | SFMBT2 | DSG2 |
| 658 | TMEM108 | RAB3B | PDSS2 | SOX11 | KCNN2 | NAP1L3 |
| 659 | DISP1 | UBXN7 | SUGCT | SLC6A8 | BCAS3 | SOX11 |
| 660 | PHLDB2 | FZD7 | MLLT3 | BCLAF1 | ZNF254 | KDELR1 |
| 661 | ME1 | THUMPD1 | ACSS3 | TMED10 | KIRREL | PCNA |
| 662 | TFPI | VAT1L | KIAA1109 | RPL7L1 | LOC107986022 | GMFB |
| 663 | LDAH | PGD | SRBD1 | NRBP1 | LOC107987083 | ARPC3 |
| 664 | DENND1B | GPC4 | NFIB | ARPP19 | POLA1 | ARPP19 |
| 665 | VGLL4 | KRAS | TAF3 | SNRPC | GSTCD | TCOF1 |
| 666 | TPST1 | HSPE1 | RABGAP1 | FEM1B | RHBDD1 | BUB3 |
| 667 | ELP4 | C1QBP | E2F3 | CHD8 | DTNB | TMEM14C |
| 668 | TNRC18 | SLC7A3 | CNKSR2 | PPT1 | LGR4 | SRSF2 |
| 669 | ANKRD17 | ACIN1 | ATG7 | LAPTM4A | SCAI | TFRC |
| 670 | WWC2 | BUB1B | ZNRF3 | POLR2A | ZNF385B | SEPT7 |
| 671 | LOC101928570 | PHB2 | TNIK | C11orf31 | EPHA6 | ENY2 |
| 672 | PPM1H | FGFR2 | KANK1 | CLIC1 | CDH2 | FAM60A |
| 673 | LINC00922 | NDUFB9 | CELF2 | THUMPD1 | DCLK1 | SRSF6 |
| 674 | LINC01278 | CDC6 | UBE2W | EIF1B | REV3L | RBM25 |
| 675 | GULP1 | SAMHD1 | ATAD2B | HIST1H2AE | NINL | DNMT1 |
| 676 | GPD2 | COX7A2 | NHS | HADHA | TMEM161B | CAPZA1 |
| 677 | EHBP1 | PPP2R2B | ZDHHC21 | EIF3E | MANBA | PCDH18 |
| 678 | UVRAG | KDM4A | PHF21A | RBM8A | HIVEP1 | RARS |
| 679 | SMAD2 | STMN1 | KCNN2 | BPTF | PPP2R3A | IL6ST |
| 680 | ATAD2B | GNPDA1 | SCN8A | BAZ2A | INPP4A | HNRNPC |
| 681 | EEA1 | BAZ2A | CENPP | COX7B | EVI5 | HMMR |
| 682 | ASAP2 | TCEB2 | SCAF8 | CTSV | CEP83 | EIF3K |
| 683 | SNX13 | ZMYND8 | FHOD3 | DCP2 | ANKS1A | HIST1H1E |
| 684 | QKI | BCCIP | SFMBT2 | MCL1 | FRS2 | UGDH |
| 685 | TNFRSF21 | KIAA0101 | AUH | CTNNB1 | LINC00693 | PSMD11 |
| 686 | NR3C2 | BEND3 | SLX4IP | TSPAN6 | ARIH1 | ANLN |
| 687 | ST5 | POLR2A | PDE3A | DNAJA2 | GRIK4 | NUP50 |
| 688 | PUM2 | FAM98A | POU2F1 | TOMM7 | SCAPER | GNL3 |
| 689 | CLIP1 | GART | LOC105370504 | REST | LYPLAL1 | CDCA7L |
| 690 | TNKS | CSRP2 | PCDH11X | HIST1H3E | TSSC1 | EIF3F |
| 691 | ZFHX3 | VAT1 | IMMP1L | TUBA1A | FAM13B | KHDRBS1 |
| 692 | CYR61 | RPL22L1 | WRN | ICMT | ANO6 | CLIC1 |
| 693 | LOC102723568 | KDELR1 | RUNX1T1 | UGDH | RBM26 | QPRT |
| 694 | CDK13 | ALDH7A1 | GAB1 | ATP1A1 | FUT8 | PHB2 |
| 695 | GAB1 | MSH2 | NEK1 | UBL5 | ARHGEF7 | COX5A |
| 696 | MON2 | COA4 | PARK2 | CDC6 | SLC24A2 | CNOT1 |
| 697 | ASIC2 | FUBP1 | USP34 | MYH10 | AFF1 | NRAS |
| 698 | GPC3 | PCNP | ZZZ3 | FABP5 | MED27 | API5 |
| 699 | LOC284825 | PPP2CA | SORBS2 | AIF1L | ZZZ3 | LTA4H |
| 700 | SENP7 | TCF3 | SPAG16 | AP1S2 | FSD1L | CCDC47 |
| 701 | ST7 | COX5A | TANC2 | SMARCC1 | CECR2 | NOL7 |
| 702 | DTWD2 | UBAP2L | KIF16B | NRAS | ADAM22 | ZFP36L2 |
| 703 | EPC2 | VMA21 | POLQ | ARPC5 | CELF2 | PSAT1 |
| 704 | GALNT10 | SRRM1 | LHFP | GLUL | SSH2 | ASPM |
| 705 | DGKH | YARS | CCDC171 | CKAP2 | IMMP1L | DANCR |
| 706 | DTNA | TMEM261 | FAM189A1 | CDC37 | P3H2 | GLUL |
| 707 | R3HDM1 | ENY2 | ZNF292 | FAM60A | MICAL3 | MAPK6 |
| 708 | BIRC6 | NLN | RAI14 | NHP2 | ZNF148 | FUBP1 |
| 709 | ZRANB3 | TIMM13 | SDK2 | SNRPA1 | THADA | CXADR |
| 710 | PLCB4 | EIF3B | GLS | VDAC1 | BMT2 | WBP11 |
| 711 | DERA | C11orf31 | GPM6B | HIST1H2AC | PPP1R9A | QSER1 |
| 712 | MAP4K4 | LUC7L3 | CACHD1 | YWHAH | HDAC8 | USP1 |
| 713 | PCDH7 | MAD2L2 | SORCS1 | CUTA | SH3GL2 | NDUFV2 |
| 714 | CHN2 | EIF1AX | CDK13 | FAM98A | PPM1H | ZNF106 |
| 715 | LOC105370108 | RCN2 | REPS1 | RPL22L1 | OPHN1 | NRBP1 |
| 716 | HEG1 | GMFB | DGKH | PRPF40A | GRIK2 | THUMPD1 |
| 717 | LRRFIP2 | TXLNG | VWDE | EIF3F | ASH1L | SLC6A8 |
| 718 | RAPGEF1 | HMGN2 | ADAMTS6 | NME4 | BICD1 | PSMC3 |
| 719 | H2AFY2 | HEATR1 | CCDC150 | LSM7 | ITPR1 | APOE |
| 720 | SUGCT | SEPW1 | GRHL2 | ARPC3 | STAU2 | OTX2 |
| 721 | PLOD2 | ICMT | RRAS2 | SMS | SLC36A4 | SPARC |
| 722 | SDK1 | EIF3K | SLC35F3 | APRT | WRN | NDUFB9 |
| 723 | DIAPH2 | TSR1 | AFF2 | PSMD11 | LOC107986770 | RBBP7 |
| 724 | GSE1 | TMED2 | LRCH1 | HIF1A | TENM2 | DNAJC7 |
| 725 | DIS3L2 | PNN | ST7 | CAND1 | FGGY | BAZ2A |
| 726 | DCAF6 | HMGN5 | NXN | HIST1H3I | MAP4K5 | HIST2H2AC |
| 727 | PHKB | DNAJC7 | MAP2K5 | CYP2S1 | SVIL | RCN2 |
| 728 | USH2A | SLC7A8 | LIMS1 | PRDX5 | FHOD3 | NDUFB10 |
| 729 | ZCCHC7 | TPX2 | DEPDC1B | LARS | IKZF2 | GPI |
| 730 | GPC5 | TAOK1 | CDH13 | COA4 | SLC39A11 | DYNC1H1 |
| 731 | FCHSD2 | NDUFS6 | ATP11A | RBM12 | UCK2 | UBAP2L |
| 732 | MAST4 | GPATCH4 | NPSR1-AS1 | CXADR | KCNH7 | TCEAL4 |
| 733 | SLC36A4 | EIF2AK2 | CDH6 | UBAP2L | GLS | VDAC1 |
| 734 | WDR7 | MARCKS | SENP6 | PRDX3 | GRAMD1B | PRDM14 |
| 735 | LARP4B | BEND4 | RPTOR | PSMD8 | PCLO | ACP1 |
| 736 | DENND5B | CCDC47 | PDE7A | GPI | POU2F1 | NDUFS6 |
| 737 | MAP2 | PSME3 | FNIP1 | ARF1 | RAPGEF5 | HN1 |
| 738 | CTNND2 | SHFM1 | CTBP2 | C12orf57 | MLLT3 | RPN1 |
| 739 | ADD3 | SORL1 | THADA | HIST1H2BD | ADAMTS20 | PDAP1 |
| 740 | ARHGAP24 | GPX4 | CNTNAP2 | ENSA | LAMA2 | KIAA1551 |
| 741 | DSCAM | RUVBL1 | ELAVL2 | HSPB1 | WWP1 | GNAI3 |
| 742 | KCNQ1OT1 | HCFC1 | KIAA1468 | GNAI2 | TYW1B | SHFM1 |
| 743 | BCAR3 | RRP1B | SLC36A4 | HCFC1 | LDAH | PRRC2A |
| 744 | MYCBP2 | ALPL | BIRC6 | NUP50 | PIK3C3 | PPDPF |
| 745 | MYO1B | CPSF3 | ZNF148 | COX6A1 | RAD54B | POLR2B |
| 746 | RPTOR | SEC11A | ZNF277 | HNRNPC | TNIK | RPN2 |
| 747 | CCDC102B | BEX1 | MRPS28 | DENR | FUT9 | EZR |
| 748 | TSSC1 | NME4 | SIPA1L2 | ARPC2 | SLCO3A1 | PRDX3 |
| 749 | USP3 | LARP1 | LCLAT1 | PTPN11 | AEBP2 | LSM3 |
| 750 | TRABD2B | PCBP2 | ARHGEF7 | BEX1 | DENND4C | FEM1B |
| 751 | KHDRBS2 | CARHSP1 | GSTCD | ACIN1 | DOCK1 | RBM27 |
| 752 | SEMA5A | FEZ1 | TDRP | CNIH4 | CTTNBP2 | PRDX5 |
| 753 | TDRD3 | C12orf57 | CTDSPL | RAD50 | ARHGEF11 | RBM12 |
| 754 | VRK2 | WNK3 | ZNF519 | VMA21 | LOC101928096 | CUTA |
| 755 | ST3GAL1 | WBP11 | CUL3 | POMP | NUTM2B-AS1 | PSMD8 |
| 756 | KCNH8 | TMEM14C | RAD54B | SRSF6 | ERCC8 | HIST1H1C |
| 757 | PLAS1 | SMS | LOC100506990 | ERP29 | LCOR | SNRPC |
| 758 | MYO3A | NCAPD2 | TTTY14 | TPX2 | STAC | PPP1CB |
| 759 | NEK1 | TXN | LRIG1 | MAD2L2 | PDE10A | LUC7L3 |
| 760 | DLEU2 | ACP1 | SLC39A11 | GNPDA1 | EVL | TIMM13 |
| 761 | TRIM44 | SNRPC | HS6ST2 | BUB3 | NAALADL2 | ACIN1 |
| 762 | KIF16B | ADGRV1 | GRM8 | YME1L1 | NCAM2 | SMC2 |
| 763 | CLEC16A | CLTA | FOXJ3 | VRTN | COL23A1 | COPB1 |
| 764 | LINGO2 | SRP72 | LOC645513 | ABCE1 | PTCHD4 | CDC37 |
| 765 | SEMA3C | ATP5F1 | RBM33 | TMEM261 | SPRED2 | CNIH4 |
| 766 | SMC5 | UBE2K | SLCO3A1 | P4HB | LOC105377860 | TNP01 |
| 767 | MLLT10 | TXNDC17 | TLE1 | IDI1 | THRB | PTP4A2 |
| 768 | THBS1 | ZC3H15 | ADARB2 | RPN1 | 3-Mar | TAOK1 |
| 769 | REEP3 | CNIH4 | PPP2R3A | NCBP2 | RYBP | NCAPD2 |
| 770 | ZNF254 | SNRPB | CNTN1 | SERP1 | ARHGAP10 | GPX1 |
| 771 | NRG1 | CNN2 | MON2 | NDUFB11 | CUX2 | APRT |
| 772 | ATF7IP2 | SRSF6 | RNF217 | TOR1AIP2 | ARMC8 | P4HB |
| 773 | ASXL3 | ARMCX2 | LOC107985962 | QPRT | CDC73 | PNN |
| 774 | NCOA1 | MRPS21 | BRIP1 | TMEM258 | ZNF767P | NAA15 |
| 775 | NTNG1 | EIF4H | DACH1 | KIAA1551 | EPHA7 | TMPO |
| 776 | SRGAP2 | MCM7 | AGFG1 | AHCY | LARP1B | SNRPB |
| 777 | BRAF | KDM1A | RNF38 | NDUFA6 | DMD | CTNNA1 |
| 778 | ENC1 | SBK1 | C1QTNF3-AMACR | HIST1H2BC | KCNIP4 | FAM98A |
| 779 | KTN1 | POLR1D | CWC27 | GPATCH4 | ANKRD36 | KRAS |
| 780 | ADAMTS19 | NUSAP1 | NOS1AP | SUGT1 | AVL9 | POLR2L |
| 781 | STXBP4 | UQCR10 | MAST4 | TSR1 | KIAA1958 | PMAIP1 |
| 782 | NUTM2B-AS1 | SPINT2 | NCOA1 | SCG3 | HS6ST2 | YWHAH |
| 783 | ARHGAP21 | DAB1 | ERCC8 | NUTF2 | XXYLT1 | HACD3 |
| 784 | SGMS1 | HDLBP | RHBDD1 | ROMO1 | FBX025 | NCBP2 |
| 785 | LOC107985037 | CHML | FAM13B | HN1 | ACTR3B | H2AFV |
| 786 | LDLRAD4 | SMC2 | ANKRD28 | MOB1A | HSD17B12 | ARF1 |
| 787 | STRN3 | HMGCS1 | TRABD2B | PEG10 | DACH1 | EEF1D |
| 788 | TBL1X | TXNRD1 | SASH1 | FXR1 | FBXW11 | GNPDA1 |
| 789 | LDB2 | HIST1H2AJ | MAP2 | BST2 | RGL1 | DNAJA2 |
| 790 | ARHGEF12 | LSM7 | KIAA1211 | LMAN1 | KIF16B | PWAR5 |
| 791 | SUCLA2 | ARPP19 | MAP4K5 | SNRPB2 | EMSY | SERP1 |
| 792 | TSHZ2 | FOXO1 | LOC101928437 | EZR | LCLAT1 | ENSA |
| 793 | ISPD | HIST1H2AM | CCDC141 | EIF1AX | SIPA1L2 | TXNDC17 |
| 794 | RCOR1 | EEF1D | EXOC2 | MRPL12 | CHCHD3 | TOMM6 |
| 795 | FRMD4B | SSR3 | PHKB | RARRES2 | TLN2 | TPM1 |
| 796 | MEF2C | SUB1 | DIS3L2 | MRPL3 | LOC105377862 | CTSV |
| 797 | EPB41L4A | ATP6V1G1 | TENM2 | GPX1 | DNAJB4 | UBE2Q2P2 |
| 798 | HIVEP1 | CAND1 | AFF1 | DDX46 | RNF150 | TPM2 |
| 799 | HMBOX1 | SACS | WBSCR17 | PDCD5 | CDH11 | EIF1B |
| 800 | ATP11A | RBBP7 | NUTM2B-AS1 | ARL14EPL | CCDC150 | SMARCA4 |
| 801 | SPARC | USP1 | FOXO1 | PCDH18 | SPATA6 | HSPB1 |
| 802 | PELI2 | DNMT3A | LOC728755 | NOP10 | MCU | HCFC1 |
| 803 | PELI1 | ZNF121 | LUZP2 | MED1 | WWC1 | RBM8A |
| 804 | ANKS1B | SAP18 | LOC107985037 | RBBP7 | PREX2 | PSMB5 |
| 805 | NOVA1 | SMC4 | GRIK2 | NPC2 | CACNB2 | UBA2 |
| 806 | ZAK | CHCHD2 | USP32 | ITM2C | SH3BGRL2 | INPP5F |
| 807 | HACE1 | DDX24 | SIPA1L3 | RBM25 | LOC107986021 | VMA21 |
| 808 | PSPC1 | SLC1A5 | LINC00693 | BIRC5 | GRIA1 | TOR1AIP2 |
| 809 | AGTPBP1 | TOMM7 | STXBP4 | ANAPC11 | FREM1 | BMS1 |
| 810 | PCNX1 | MTHFD1 | CHN2 | TOMM5 | ERC1 | PTTG1 |
| 811 | PAN3 | PSMD4 | SLC24A3 | MAPK6 | LOC107985037 | ZC3H13 |
| 812 | ARHGEF10 | MAPRE1 | FSD1L | PTP4A2 | GPR176 | BIRC5 |
| 813 | CBR4 | HIF1A | OSBPL8 | SMARCD1 | SLC30A7 | ITM2C |
| 814 | FNIP1 | HSBP1 | OPHN1 | PSMB5 | RGS17 | TOMM7 |
| 815 | ITCH | BMS1 | XRCC4 | HMMR | GRM8 | PHGDH |
| 816 | MAP1B | RMND5A | FBXO11 | ATP51 | CBFA2T2 | SAP18 |
| 817 | PDGFD | CALU | MARCH3 | CSRP2 | MAST2 | FSTL1 |
| 818 | PRKD3 | ZNF649 | PREX2 | CNOT1 | CCDC141 | COA4 |
| 819 | CHCHD3 | PGRMC2 | NEGR1 | TUBB4B | ANKRD28 | MSH2 |
| 820 | CCDC171 | PDK3 | MICAL3 | AURKAIP1 | ACBD6 | TMEM261 |
| 821 | TBCA | SOX11 | SSBP3 | LAPTM4B | UBTD2 | C11orf31 |
| 822 | ATXN7 | ERP29 | PTPN14 | RRM2 | HIVEP2 | C12orf57 |
| 823 | CTGF | AKR1A1 | MANBA | EIF2AK1 | LOC105370108 | IDI1 |
| 824 | PRDM2 | KARS | ASH1L | SAR1A | LOC107983984 | SNRPA1 |
| 825 | RASSF8 | HADHA | NFAT5 | PTMS | RAI14 | NUTF2 |
| 826 | PAPPA | KPNA6 | SRGAP1 | EEF1D | NTNG1 | VCL |
| 827 | SUCO | TTF2 | NCK2 | HMGN5 | CMTM8 | PPT1 |
| 828 | C11orf49 | FGFBP3 | RSU1 | BANF1 | IL1RAPL1 | AKAP12 |
| 829 | ARRB1 | GNAI2 | MCU | TPM2 | CRADD | RAB10 |
| 830 | NME7 | POLR2L | GSE1 | NDUFB7 | PLEKHG4B | PPA1 |
| 831 | C20orf194 | ADD2 | ZNF254 | IL6ST | LOC105377700 | GNAI2 |
| 832 | LOC107986324 | ZNF850 | RALGAPA1 | UQCR11 | CAMK1D | HUWE1 |
| 833 | ELK3 | BZW1 | MAN1A2 | RPN2 | NK2 | PCM1 |
| 834 | LOC105378797 | EIF2AK1 | FAM169A | CTR9 | TDRD3 | CTR9 |
| 835 | UBR3 | SNRPA1 | WWP1 | MRPS16 | SOS1 | SMC1A |
| 836 | CHRM3 | RNF20 | IPO11 | PPA1 | SMURF1 | TUBB4B |
| 837 | GREB1 | CRABP1 | LAMA2 | IK | FAM110B | CHEK1 |
| 838 | S100A10 | COX7B | PCNX4 | MRPS21 | EFCAB2 | C1QBP |
| 839 | GSTCD | HIST1H3E | MYO9A | MCM7 | SGK223 | SARAF |
| 840 | PITPNC1 | CDC37 | PRR16 | SLC1A5 | WWTR1 | SAR1A |
| 841 | GLCCI1 | PSMC5 | INTS6 | FZD7 | SYT14 | RRM1 |
| 842 | SEC63 | MARS | TMEM161B | UBXN7 | FOXO1 | NME4 |
| 843 | KSR1 | ZNF589 | MGMT | HIST1H2AL | MAP2 | BUB1B |
| 844 | KIAA1211 | HIST1H2BD | TPST1 | PPP2CA | SDCCAG8 | LSM7 |
| 845 | ACVR1 | RNF168 | ANTXR1 | IFITM1 | ANKRD17 | CCNC |
| 846 | MAP2K5 | MTA3 | ARHGAP10 | MAGED2 | PELI1 | PSMA6 |
| 847 | CENPP | DMKN | JAKMIP2 | HIST1H2AH | AGFG1 | UBE2N |
| 848 | DYNC111 | KIF20B | FUT9 | ANP32B | PUM2 | CDK1 |
| 849 | LINC01356 | FAM111B | PLEKHG4B | AMD1 | NSUN6 | RAB1A |
| 850 | H2AFY | FDFT1 | SDCCAG8 | AARS | VPS13A | RPL22L1 |
| 851 | DENND2A | ESRP1 | TSC22D1 | ARMCX2 | PKNOX2 | ATP1A1 |
| 852 | CHSY3 | DCAF13 | C11orf49 | STARD7 | MAN2A1 | NDUFB11 |
| 853 | RFTN2 | MAPK6 | PARGP1 | PHGDH | SLC35F1 | GART |
| 854 | ABCG2 | UNG | GAB2 | H2AFV | ANO4 | SESN3 |
| 855 | CEP170 | ZNF397 | LOC101929194 | CDCA7L | CNOT4 | EIF2AK2 |
| 856 | RHBDD1 | MED1 | CECR2 | MRPL15 | VAV3 | ICMT |
| 857 | SLC35F3 | ASNS | TTC3 | FKBP2 | ARHGEF9 | MOB1A |
| 858 | CACNA2D1 | SOLE | AHNAK | EIF3B | MOB3B | SOX4 |
| 859 | USP34 | ATP1B3 | PDE10A | TRMT10C | LRP12 | PRKAR1A |
| 860 | SIK3 | PRRC2A | TSSC1 | RARS | XKR4 | UBXN7 |
| 861 | KAT6A | EIF3F | TMEM131 | NGRN | PAT | UBE2V2 |
| 862 | NCK2 | TOP2B | YAP1 | PTP4A1 | ST6GAL1 | CSRP2 |
| 863 | RNF13 | AKR1B1 | LCOR | EBNA1BP2 | COL4A2 | TMA7 |
| 864 | FBXO11 | PCDH1 | FCHSD2 | NDUFB4 | CSPP1 | COX7B |
| 865 | FTX | NACC1 | FBXW11 | ACAT2 | PDLIM5 | DNMT3A |
| 866 | TSC22D2 | CLNS1A | TBCD | TUBA1C | RAP1GAP2 | MAD2L2 |
| 867 | ADGRB3 | ZNF207 | NRF1 | GTF3C4 | LINC01278 | NHP2 |
| 868 | LOC101928437 | COPB2 | GRIA1 | CARHSP1 | SNX25 | KIF20B |
| 869 | NLK | SCNN1A | AHI1 | SMC2 | AGTPBP1 | MDN1 |
| 870 | PIK3R1 | STARD7 | BMT2 | NDUFB8 | ROR2 | GPATCH4 |
| 871 | THSD7A | PTPN11 | SDC2 | SARAF | AP3B1 | CDC6 |
| 872 | NXN | SMC1A | ANKDD1A | FGD5-AS1 | LOC100287497 | ZYG11B |
| 873 | ZZZ3 | TOR1AIP2 | NME7 | MRPL57 | RALGAPA1 | ABRACL |
| 874 | CSPP1 | PRKAR2B | DENND5B | RNF168 | STXBP6 | RAD23B |
| 875 | ERC2 | ABCE1 | PKD2 | RUVBL1 | MICU3 | CCAR1 |
| 876 | LOC645513 | BOD1 | PPP2R5E | SLC39A1 | BARD1 | ADSS |
| 877 | SAMD5 | DNAJA2 | ROR2 | SPCS3 | TNKS | WDR1 |
| 878 | PKD2 | LSM3 | TBC1D4 | DNAJC7 | STK39 | IK |
| 879 | COL4A6 | JADE1 | USP6NL | C9orf78 | LHFPL2 | MED1 |
| 880 | DMTF1 | CUTA | KANSL1L | C19orf53 | AMBRA1 | RRBP1 |
| 881 | CEP83 | ATP1A1 | SORBS1 | GART | PACS1 | COX6A1 |
| 882 | ANTXR2 | MRPS16 | PLAS1 | PSAT1 | ITCH | AURKAIP1 |
| 883 | ERCC8 | ELP2 | CDK5RAP2 | DAZAP1 | CEP192 | RBBP4 |
| 884 | LMBR1 | PRDX5 | CDH8 | CPSF3 | DIS3L2 | SEC11A |
| 885 | ST6GAL1 | HUWE1 | STX18-AS1 | GTF3C6 | ZDHHC17 | CDC20 |
| 886 | FBN1 | CDC123 | PPA2 | MDH2 | SRBD1 | RRP1B |
| 887 | BMP2K | API5 | LINC01619 | NEDD8 | CASK | CPSF3 |
| 888 | SNX14 | TIMELESS | RNF13 | BMS1 | SDC2 | NRDC |
| 889 | SLC44A5 | TRMT10C | WAC | CDC20 | TBC1D4 | SCG3 |
| 890 | FAM135A | TNPO3 | USP9Y | MAP1LC3B | STARD4-AS1 | CYP2S1 |
| 891 | FAM168A | DDX42 | CLOCK | ABRACL | LOC101929709 | EIF1AX |
| 892 | ANO10 | HSPB1 | DOCK7 | NUSAP1 | KHDRBS3 | MYL12B |
| 893 | POU6F2 | SLC39A1 | NINL | ZMAT2 | CNOT2 | ANAPC11 |
| 894 | LOC107983984 | DAZAP1 | LOC107985961 | DCTN1 | CCDC171 | VRTN |
| 895 | CHSY1 | CACHD1 | ADAM22 | RBM27 | C5orf46 | PSMD6 |
| 896 | ZMYND11 | CAMLG | SCAI | MIDN | NAA16 | DCAF13 |
| 897 | HIBCH | GTF3C4 | BCL11A | NDUFAB1 | LOC101929147 | CDC123 |
| 898 | ZDHHC21 | SYT6 | JPX | ATP6VOC | RAP1A | MRPL12 |
| 899 | LOC105378798 | ALDH1B1 | RIC8B | SNX6 | LOC105375334 | POMP |
| 900 | BZW2 | AGO2 | CHM | GNL2 | JAKMIP2 | UBL5 |
| 901 | IMMP1L | DNMT1 | SLC44A5 | AKR1B1 | CENPP | TM9SF2 |
| 902 | EPHA4 | CTSV | NRCAM | GMNN | TAF4B | PTTG1IP |
| 903 | CUL3 | MIS18A | EFR3A | USMG5 | NHS | PDCD5 |
| 904 | TBC1D23 | SLC16A9 | MAP4K4 | PSMA6 | ZDHHC21 | TMEM258 |
| 905 | FAM13B | NDUFB7 | ANKRD18CP | LMNB2 | ALCAM | IPW |
| 906 | SLC7A11 | SPTBN2 | MTAP | NDUFA3 | TMEM131 | U2SURP |
| 907 | ADARB1 | NUP62 | NCAM2 | SMC4 | FLRT2 | EIF3B |
| 908 | ARHGAP6 | MIS18BP1 | CENPK | RRP1B | TRERF1 | ATIC |
| 909 | ADAMTS9 | MCM5 | SERINC5 | ECHS1 | NRG1 | CNDP2 |
| 910 | HIPK2 | ANOS1 | TRERF1 | TIMM8B | SRGAP1 | ARL14EPL |
| 911 | NRF1 | HYOU1 | SLC25A13 | EIF3D | DNM3 | TROVE2 |
| 912 | CAPN2 | MKNK2 | HECW2 | PSMC2 | GRIN2B | KDM1A |
| 913 | NUAK1 | FTSJ3 | CSPP1 | APIS | GPR137C | DLD |
| 914 | LRRC8D | FABP5 | PDE5A | MRPL52 | NHSL2 | AIF1L |
| 915 | CDC14A | LSG1 | FAM19A5 | ZYG11B | BNC2 | NSD1 |
| 916 | MTR | ATP5J2 | STIM2 | RND2 | LOC100506990 | TCEA1 |
| 917 | USP15 | SIN3A | TENM1 | MRPS15 | NXN | UBQLN1 |
| 918 | GPM6B | NGRN | ROCK2 | QSER1 | NME7 | CTSC |
| 919 | MDFIC | TRIM59 | CDC14A | HAUS1 | XRCC4 | NDUFB8 |
| 920 | CDC73 | CCDC6 | PTPR | CCNC | LOC105370504 | YARS |
| 921 | PTPN12 | MTCH1 | NT5C2 | CWC15 | PDE5A | SMARCD1 |
| 922 | NETO2 | ALDH6A1 | RALGPS2 | CNDP2 | RNF130 | FABP5 |
| 923 | DYNC2H1 | ZNF134 | CRADD | HIST1H3F | COL11A1 | ERP29 |
| 924 | EPC1 | QPRT | CHD7 | SPINT2 | DACH2 | GRSF1 |
| 925 | FLNB | NHP2 | CWF19L2 | MAL2 | BRE | ATP1B3 |
| 926 | MRPS28 | RFC1 | LOC105374945 | KIAA0101 | ZC3H12C | EBNA1BP2 |
| 927 | WWP1 | ATP6VOC | ZNF618 | IDO1 | GABBR1 | ARMCX2 |
| 928 | TGFBR3 | TCEAL4 | CEP112 | RAB1A | PPP3R1 | NGRN |
| 929 | MARK1 | IVD | MPP6 | NOP58 | PBX3 | NPC2 |
| 930 | IKZF2 | SEC61B | LINC-PINT | DPY30 | ACVR1 | TRAM1 |
| 931 | SCAF8 | MFAP1 | LYPD6 | CAPZA1 | PDE3A | BEX1 |
| 932 | SNRPN | FKBP8 | CBR4 | FTSJ3 | CSNK1G1 | UBE2K |
| 933 | SLC25A13 | TRMT112 | LARP1B | PHAX | TANC2 | TSR1 |
| 934 | BMT2 | PDIA4 | VPS41 | PSMD6 | LOC107985962 | NDUFAB1 |
| 935 | C9orf84 | RANBP1 | ATF7IP2 | STOML2 | SPAG16 | SPCS3 |
| 936 | MECOM | ADSS | ATXN7 | OIP5-AS1 | GCNT2 | TXNL1 |
| 937 | DENND5A | ANAPC11 | KCNIP4 | RBBP4 | LINC01122 | ARHGAP11A |
| 938 | OSBPL3 | NEDD8 | GPR137C | EI24 | SHB | IMMT |
| 939 | ZFHX4 | ACTR2 | INPP4A | HIST1H4D | SUGCT | DAZAP1 |
| 940 | CHM | FASN | MAST2 | KPNA6 | LOC100505817 | RUVBL1 |
| 941 | ELOVL6 | XPO5 | PLPP3 | CDC123 | PRRG1 | PTMS |
| 942 | TBL1Y | DYNLL2 | ATF2 | UBE2V2 | TANC1 | NDUFA6 |
| 943 | SUMF1 | HAUS1 | ARHGAP44 | NUP62 | CPNE8 | HAUS1 |
| 944 | STARD13 | LINC00665 | CNOT4 | UBA2 | RASSF8 | CTTN |
| 945 | RGL1 | DPY30 | NXPH2 | C14orf2 | KIF13A | DPYSL2 |
| 946 | DAPK1 | SV2A | PPP1R12B | SUMO3 | CDK6 | SEC62 |
| 947 | MAP3K4 | NRBP1 | FRS2 | BRK1 | ATXN7 | STK26 |
| 948 | HIVEP2 | OSTC | SYN3 | KIF11 | NEK1 | GNPTAB |
| 949 | LATS2 | ARCN1 | DLEU2 | CDK2AP1 | CHM | BST2 |
| 950 | LRP12 | HOOK1 | ATE1 | KRAS | EYA1 | ANAPC5 |
| 951 | ZFYVE16 | UPF3B | CTIF | MYL12B | NFIA | LMNB1 |
| 952 | DISC1FP1 | DSTN | ZHX2 | BUB1B | SUCLG2 | HNRNPUL1 |
| 953 | PTEN | CDK2AP1 | ARHGEF12 | BOD1 | CACNA1A | GNL2 |
| 954 | STX8 | SRRT | SLC25A26 | COX6C | NCOA1 | SPINT2 |
| 955 | SLC25A24 | PHC1 | KSR1 | DDB1 | CNTN1 | VDAC2 |
| 956 | NRP2 | TTK | EHMT1 | INPP5F | LOC105379362 | LMNB2 |
| 957 | MAST2 | EI24 | MOB3B | TUFM | GALNT18 | SRSF5 |
| 958 | ASPH | UBL5 | RCOR1 | BCAS2 | TNFAIP8 | TRIM59 |
| 959 | SEC24B | SMARCA4 | LOC105375334 | DCAF13 | VGLL4 | BAZ1B |
| 960 | AHI1 | TGIF1 | RNF220 | DNMT1 | MTCL1 | ARF4 |
| 961 | TBC1D19 | ZNF814 | ZNF124 | RAC1 | GMDS-AS1 | DDB1 |
| 962 | SNCA | LMNB2 | LOC107986215 | DYNLL2 | ADAMTS6 | RRM2 |
| 963 | STK39 | RPN2 | REV3L | RNF7 | CHN2 | WNK3 |
| 964 | STPG2 | CKAP2 | LOC105370108 | TRMT112 | CEP112 | FKBP2 |
| 965 | PACS1 | CTR9 | SLC20A2 | RRM1 | RASGRF2 | USP14 |
| 966 | RBM20 | RFWD3 | PGAP1 | CLNS1A | TSC22D2 | RDX |
| 967 | ZNF91 | WDFY1 | ZNF644 | GHITM | CTIF | FERMT2 |
| 968 | SMG6 | PSMD11 | VRK2 | PBDC1 | LOC105375751 | MAD2L1 |
| 969 | PEAK1 | PSMC3 | TANC1 | SRRT | ACSS3 | SBNO1 |
| 970 | SH3D19 | ADSL | IKZF2 | HIST1H3H | EPB41L5 | EIF2AK1 |
| 971 | LOC101928096 | CNDP2 | ATXN10 | STK26 | LYPD6 | NDUFB7 |
| 972 | ZHX2 | GSPT2 | EMSY | PHPT1 | RCOR1 | KPNA6 |
| 973 | USP24 | HSD17B4 | HIVEP2 | KRT19 | LINC01057 | SNRPB2 |
| 974 | FANCL | SAE1 | KHDRBS3 | EIF2S1 | QKI | RARRES2 |
| 975 | FOXP2 | BRD3 | ITCH | CHML | MON2 | NEDD8 |
| 976 | PPP2R5E | GMNN | UBR3 | ODC1 | TRAK1 | BANF1 |
| 977 | ARHGEF7 | AURKAIP1 | NECTIN3 | MIS18A | XPR1 | PSMC2 |
| 978 | ASH1L | SF3B3 | DCLK1 | MAD2L1 | BTBD11 | SSR1 |
| 979 | RNF24 | YWHAH | PAWR | SSR4 | HIBADH | RAC1 |
| 980 | AFF2 | TUBA1C | SNX9 | KHDRBS1 | SORBS1 | RNF7 |
| 981 | TRAM2 | U2SURP | ASAP2 | ARF4 | SRGAP3 | ACTR3 |
| 982 | GRIN2A | RPL7L1 | PLCL2 | ANXA5 | DENND5B | NOP10 |
| 983 | CNTLN | DAXX | ARFIP1 | HACD3 | RNF217 | OIP5-AS1 |
| 984 | LCLAT1 | CTNNB1 | GAREM1 | PWAR5 | SHPRH | ECHS1 |
| 985 | DAAM1 | CCAR1 | TRPM7 | EIF4A3 | ARFIP1 | MRPL15 |
| 986 | SH3BGRL2 | EZR | LOC102724001 | VBP1 | PCDH11Y | PFN2 |
| 987 | EVL | ZIC5 | CEP170 | NUDCD2 | PARK2 | RNF20 |
| 988 | PIK3CB | PTTG1 | EPC1 | C6orf62 | CLOCK | MRPS16 |
| 989 | TRAK1 | TMEM258 | VGLL4 | SV2A | KCMF1 | RMND5A |
| 990 | MTAP | ARL14EPL | TRAF3IP2-AS1 | GRSF1 | DNAH11 | MRPS15 |
| 991 | KPNA1 | ATP5C1 | ZNF767P | HNRNPUL1 | PIAS2 | UQCR11 |
| 992 | DIP2B | NUP50 | AVL9 | SLC7A3 | GPM6B | TMEM14B |
| 993 | SMAD6 | WDR43 | CSMD1 | UNG | LATS2 | LRPPRC |
| 994 | ZNF277 | NRDC | SLC30A7 | DAXX | HMCN1 | CDK12 |
| 995 | XRN1 | NDUFB11 | TDRD3 | CTSC | POLQ | FGD5-AS1 |
| 996 | FOX]3 | PAIP2B | BRINP1 | PRKAR1A | ANKRD6 | DDX42 |
| 997 | SHPRH | PIM2 | POU6F2 | YARS | SEC24D | SEPT10 |
| 998 | SMURF1 | SALL3 | CUX2 | DDX27 | LOC101929563 | MAP1LC3B |
| 999 | LYPD6 | CD63 | EPHA6 | HEATR1 | POU6F2 | PTP4A1 |
| 1000 | CHD9 | TUFM | FANCC | AP2S1 | C11orf49 | STARD7 |

TABLE 17
Splice isoform annotations and differentiation outcome. Splice isoform annotations for 9 TF genes
indicating differences between isoforms and whether domains that may be important for function
are missing. The diffusion pseudotime P-values are included for each isoform. AA, amino acid.

| | | | | | Missing |
|---|---|---|---|---|---|
| | Pseudotime | Length | Splice isoform difference | | important |
| TF ORF | P-value | (AA) | relative to consensus | Important domains in consensus | domains? |
| TFORF0983-KLF5 | 8.09E−55 | 457 | Consensus | AA 324-328 interaction with | no |
| | | | | WWP1, AA 373-397 zinc finger, | |
| | | | | AA 403-427 zinc finger, AA 433- | |
| | | | | 455 zinc finger | |
| TFORF0984-KLF5 | 9.96E−46 | 366 | AA 1-91 missing | | no |
| TFORF1158- | 4.04E−01 | 705 | AA 460 insertion of 19 AA | | no |
| EOMES | | | | | |
| TFORF1159- | 2.04E−07 | 686 | Consensus | AA 276-456 DNA binding, AA | no |
| EOMES | | | | 571-686 required for | |
| | | | | transcription activation | |
| TFORF1160- | 4.64E−03 | 410 | AA 1-295 missing, AA 460 | | partially |
| EOMES | | | insertion of 19 AA | | |
| TFORF2743-T | 0.05371542 | 377 | AA 244-302 missing | | no |
| | 9 | | | | |
| TFORF2744-T | 5.37E−19 | 435 | Consensus | AA 51-219 DNA binding | no |
| TFORF1447-NR5A2 | 1.07E−07 | 469 | AA 1-72 missing | | no |
| TFORF1448-NR5A2 | 0.07477391 | 541 | Consensus | AA 83-154 DNA binding, 155-184 | no |
| | 1 | | | FTZ-F1 box, 300-539 ligand | |
| | | | | binding | |
| TFORF3131-NR5A2 | 0.00281719 | 495 | AA 22-67 missing | | no |
| | 2 | | | | |
| TFORF1513-NKX3- | 0.29313432 | 234 | Consensus | AA 124-183 DNA binding | no |
| 1 | 9 | | | | |
| TFORF1514-NKX3- | 3.93E−05 | 159 | AA 13-87 missing | | no |
| 1 | | | | | |
| TFORF1169-EBF1 | 0.00014125 | 560 | AA 162-184 missing, AA 252-259 | | partially |
| | 5 | | missing | | |
| TFORF3058-EBF1 | 0.46799205 | 591 | Consensus | AA 63-66 DNA interaction, AA | no |
| | 2 | | | 151-170 zinc finger, AA 197-204 | |
| | | | | DNA interaction, AA 236-239 | |
| | | | | DNA interaction, AA 262-345 | |
| | | | | IPT/TIG, | |
| TFORF0213-FOXP1 | 0.16910964 | 577 | AA 1-100 missing | | no |
| | 5 | | | | |
| TFORF0214-FOXP1 | 0.40157246 | 677 | Consensus | AA 306-331 Zinc finger, 348-369 | no |
| | 8 | | | leucine zipper, 382-386 CTBP1- | |
| | | | | binding, AA 465-555 DNA binding | |
| TFORF0215-FOXP1 | 0.40916766 | 676 | AA 450 missing | | no |
| | 8 | | | | |
| TFORF0216-FOXP1 | 0.46279587 | 601 | AA 95-170 missing | | no |
| TFORF0217-FOXP1 | 0.38858319 | 577 | AA 1-100 missing | | no |
| | 6 | | | | |
| TFORF0218-FOXP1 | 0.36383866 | 693 | AA 511-551 insertion | | domain |
| | 8 | | | | insertion |
| TFORF3063-FOXP1 | 0.00063486 | 114 | AA 61-114 altered, AA 115-677 | | yes |
| | | | missing | | |
| TFORF0207-FOXP2 | 0.10180136 | 432 | AA 423-432 altered, AA 433-715 | | yes |
| | 8 | | missing | | |
| TFORF0208-FOXP2 | 0.01815991 | 715 | Consensus | AA 346-371 zinc finger, AA 388- | no |
| | 5 | | | 409 leucine zipper, AA 422-426 | |
| | | | | CTBP1-binding, AA 504-594 DNA | |
| | | | | binding | |
| TFORF0209-FOXP2 | 0.02004428 | 740 | AA 86 insertion of 25 AA | | no |
| | 1 | | | | |
| TFORF0210-FOXP2 | 0.00047311 | 732 | AA 132 insertion of 17 AA | | no |
| | 3 | | | | |
| TFORF2165-GRHL3 | 2.27E−07 | 602 | AA 566-626 altered | | no |
| TFORF2166-GRHL3 | 0.03001187 | 626 | Consensus | AA 30-95 transcriptional | no |
| | 1 | | | activation, AA 226-460 DNA | |
| | | | | binding | |
| TFORF2167-GRHL3 | 0.08267995 | 607 | AA 1-6 insertion, AA 566-626 | | no |
| | 6 | | altered | | |
| TFORF2168-GRHL3 | 3.71E−05 | 556 | AA 1-46 missing, AA 566-626 | | partially |
| | | | altered | | |

TABLE 18
Cluster marker genes for differentiated cells in the TF Atlas. Cells were ordered by diffusion pseudotime relative to Cluster 0. Linear regression was applied to identify
genes that were significantly differentially expressed (FDR < 0.05) over pseudotime. The estimated slope of the linear regression fit and associated P-values are listed for each marker gene
(Each cluster is shown in the left column. The right column shows Gene 1, Slope, P-value; Gene 2, Slope, P-value; etc.).
Cluster

| | |
|---|---|
| 0 | LOC100505817, −2.4E+01, 7.6E−21; CHODL, −1.9E+01, 3.4E−09; SYT1, −1.8E+01, 1.9E−12; FGF13, −1.8E+01, 1.2E−09; LRRTM4, −1.7E+01, 5.0E−13; |
| | SEMA6A, −1.7E+01, 2.1E−15; LINC01194, −1.7E+01, 2.0E−10; LOC105377901, −1.6E+01, 1.1E−08; ABHD12B, −1.6E+01, 8.4E−11; LSAMP, −1.6E+01, |
| | 1.8E−09; TTN, −1.5E+01, 6.0E−07; SGCD, −1.5E+01, 2.6E−10; GALNT3, −1.5E+01, 3.4E−11; C9orf135, −1.5E+01, 3.4E−11; SPP1, −1.4E+01, 1.2E−08; SLC39A10, |
| | −1.4E+01, 6.0E−08; S100A10, −1.4E+01, 7.9E−10; EZR, −1.4E+01, 2.4E−07; GSTP1, −1.4E+01, 1.7E−08; KRT8, −1.4E+01, 1.0E−07; KRT18, −1.4E+01, 5.4E−07; |
| | LOC101927668, −1.3E+01, 1.4E−05; LOC105375710, −1.3E+01, 1.6E−08; BICD1, −1.3E+01, 8.6E−08; OPCML, −1.3E+01, 3.6E−06; FOXN3, −1.3E+01, 4.7E−12; |
| | JARID2, −1.3E+01, 1.0E−10; POU5F1, −1.3E+01, 3.7E−06; ST6GAL1, −1.3E+01, 1.1E−05; SKIL, −1.2E+01, 1.0E−05; L1TD1, −1.2E+01, 4.4E−12; |
| | LOC105374945, −1.2E+01, 2.0E−05; MRS2, −1.2E+01, 5.9E−06; GRID2, −1.2E+01, 2.7E−33; MDN1, −1.2E+01, 6.1E−05; RIMS2, −1.1E+01, 5.4E−07; PCAT14, |
| | −1.1E+01, 1.5E−04; RAD51B, −1.1E+01, 1.9E−04; RPSA, −1.1E+01, 1.1E−04; CACNA2D3, −1.1E+01, 2.1E−04; FGF2, −1.1E+01, 1.6E−04; PAWR, −1.1E+01, |
| | 2.1E−04; RNF220, −1.1E+01, 3.6E−04; VSIG10, −1.1E+01, 1.8E−05; LOC107987087, −1.1E+01, 1.9E−05; GRIA1, −1.1E+01, 1.1E−05; LOC102467213, |
| | −1.1E+01, 1.2E−03; IQGAP2, −1.0E+01, 2.1E−04; RESF1, −1.0E+01, 8.1E−04; PDLIM1, −1.0E+01, 3.3E−05; USO1, −1.0E+01, 6.9E−04; TMEM132D, −1.0E+01, |
| | 1.3E−03; MPPED2, −1.0E+01, 2.6E−04; ARFGEF1, −9.9E+00, 1.7E−03; TNS3, −9.8E+00, 1.1E−03; CCT3, −9.7E+00, 2.0E−03; HSPA8, −9.7E+00, 6.9E−06; |
| | LARP1, −9.7E+00, 2.6E−03; LOC101929194, −9.6E+00, 2.8E−03; OSBPL10, −9.6E+00, 2.6E−03; DPPA4, −9.6E+00, 3.6E−04; PPP1CC, −9.6E+00, 2.0E−03; |
| | DCLK1, −9.5E+00, 1.1E−03; ANKRD18CP, −9.5E+00, 1.2E−03; EIF4G3, −9.5E+00, 2.2E−03; BNC2, −9.5E+00, 1.2E−03; FLVCR1, −9.4E+00, 2.4E−03; ESRG, |
| | −9.4E+00, 1.9E−03; LOC105372310, −9.4E+00, 3.9E−03; WDHD1, −9.4E+00, 3.7E−03; VASH2, −9.3E+00, 5.3E−03; CCDC141, −9.3E+00, 2.3E−03; CD2AP, |
| | −9.3E+00, 3.3E−03; TBC1D23, −9.3E+00, 5.0E−04; G3BP1, −9.3E+00, 2.8E−03; PTAR1, −9.2E+00, 4.2E−03; LRBA, −9.2E+00, 2.8E−03; ND4L, −9.1E+00, 2.2E−03; |
| | ESRP1, −9.1E+00, 5.3E−03; TARS1, −9.0E+00, 6.5E−03; CDH13, −9.0E+00, 3.9E−04; C12orf60, −8.9E+00, 6.9E−04; RACK1, −8.9E+00, 3.9E−03; TPD52, |
| | −8.9E+00, 1.8E−03; ZNF90, −8.8E+00, 5.9E−03; TET1, −8.8E+00, 6.5E−03; TDGF1, −8.8E+00, 8.8E−03; RPL8, −8.7E+00, 3.3E−03; RYR2, −8.7E+00, 1.7E−02; |
| | JAZF1, −8.6E+00, 1.2E−02; ANXA2, −8.5E+00, 1.2E−02; ITPR2, −8.5E+00, 4.2E−03; GCNT2, −8.5E+00, 4.5E−03; ND6, −8.5E+00, 3.1E−03; TKT, −8.5E+00, |
| | 1.4E−02; PLAAT3, −8.5E+00, 1.2E−03; ZNF217, −8.4E+00, 9.3E−03; CADPS, −8.4E+00, 3.4E−03; SYNE2, −8.3E+00, 1.4E−02; MYSM1, −8.3E+00, 1.0E−02; |
| | TRIM71, −8.2E+00, 9.6E−04; CECR2, −8.2E+00, 5.8E−03; SHANK2, −8.2E+00, 2.2E−02; PCSK9, −8.2E+00, 1.6E−04; AKAP12, −8.1E+00, 2.8E−02; LINC00428, |
| | −8.1E+00, 2.1E−02; EEF1G, −8.1E+00, 2.1E−02; SERINC5, −8.1E+00, 3.6E−02; PKIB, −8.0E+00, 4.1E−02; RPS5, −8.0E+00, 2.4E−02; DNMT3B, −8.0E+00, 2.9E−04; |
| | PPARGC1A, −7.9E+00, 1.9E−02; SEMA3E, −7.9E+00, 3.2E−02; RCC2, −7.9E+00, 2.7E−02; IMMP2L, −7.9E+00, 3.4E−02; LOC107986770, −7.9E+00, 2.9E−02; |
| | STRBP, −7.9E+00, 3.8E−02; CYTB, −7.9E+00, 9.6E−07; RFX3, −7.9E+00, 3.4E−02; PRKG1, −7.9E+00, 3.9E−02; MAGI2, −7.9E+00, 4.4E−02; PPP6R3, |
| | −7.8E+00, 3.7E−02; TCF7L2, −7.8E+00, 3.2E−03; ARHGEF10, −7.8E+00, 4.3E−02; ND1, −7.8E+00, 5.2E−04; EIF4A2, −7.8E+00, 4.0E−02; PTCHD1-AS, |
| | −7.8E+00, 2.2E−02; EXT1, −7.7E+00, 4.6E−02; FOXO1, −7.7E+00, 4.8E−02; GLDC, −7.7E+00, 3.2E−03; AKAP9, −7.6E+00, 3.2E−02; RNF125, −7.6E+00, 7.6E−04; |
| | DDX5, −7.6E+00, 4.2E−02; FAM155A, −7.6E+00, 2.5E−02; IGF2R, −7.6E+00, 4.1E−02; PTMA, −7.5E+00, 5.1E−09; SLC16A1, −7.5E+00, 4.1E−02; DDX21, |
| | −7.5E+00, 4.8E−02; RIF1, −7.5E+00, 3.2E−02; CASC15, −7.4E+00, 2.9E−02; ZNF326, −7.4E+00, 1.4E−02; KIF5B, −7.4E+00, 3.9E−02; CBL, −7.4E+00, 4.4E−02; |
| | TJP2, −7.4E+00, 4.0E−02; FAM160A1, −7.3E+00, 4.4E−02; AASS, −7.2E+00, 5.6E−03; KIF5C, −7.2E+00, 2.4E−02; SLX4IP, −7.2E+00, 1.2E−02; ATP6, −7.2E+00, |
| | 1.7E−06; TPT1, −7.2E+00, 2.8E−02; COX3, −7.2E+00, 3.0E−07; PARD3B, −7.1E+00, 2.9E−02; SPG20, −7.0E+00, 4.8E−02; VRK2, −6.8E+00, 1.9E−02; ASRGL1, |
| | −6.6E+00, 2.8E−03; HSP90AA1, −6.5E+00, 2.2E−04; HNRNPU, −6.5E+00, 3.9E−02; IFITM1, −6.5E+00, 4.7E−02; HHLA1, −6.4E+00, 3.6E−02; IPMK, −6.4E+00, |
| | 4.8E−04; ARMT1, −6.4E+00, 4.7E−02; LOC107985710, −6.3E+00, 3.3E−02; LYPLAL1, −6.2E+00, 2.1E−02; C4orf51, −6.1E+00, 2.5E−02; KLF5, −5.9E+00, |
| | 1.2E−05; CCSAP, −5.8E+00, 1.1E−02; TTC39C, −5.7E+00, 4.3E−02; MT1G, −5.6E+00, 2.4E−02; F3, −5.5E+00, 9.8E−03; ZNF486, −5.5E+00, 2.2E−02; SLC25A16, |
| | −5.4E+00, 2.1E−02; NR3C2, −5.3E+00, 4.1E−02; STOM, −5.2E+00, 9.1E−03; NPM1, −5.1E+00, 1.7E−02; DOCK11, −5.1E+00, 3.5E−02; TUBB3, −5.1E+00, 4.2E−02; |
| | MIR31HG, −5.0E+00, 1.4E−02; COX2, −5.0E+00, 2.7E−02; CAVIN1, −4.8E+00, 3.9E−02; CRIP1, −4.8E+00, 5.4E−04; LOC105374827, −4.5E+00, 7.0E−03; |
| | PHKA1, −4.4E+00, 5.0E−02; BCAM, −4.3E+00, 1.8E−02; MT1H, −4.2E+00, 1.6E−02; LOC107985887, −4.2E+00, 2.1E−02; CAV1, −4.1E+00, 2.5E−03; COX1, |
| | −4.1E+00, 2.6E−03; DLL3, −4.0E+00, 2.8E−02; TRPC6, −3.5E+00, 1.9E−02; C14orf177, 9.5E−02, 1.2E−03; LOC105378566, 1.5E−01, 4.3E−03; LOC105377193, |
| | 1.6E−01, 3.9E−02; LOC107987346, 1.6E−01, 3.9E−02; LOC105377018, 1.7E−01, 2.7E−02; LOC101927606, 1.7E−01, 3.1E−03; PRR29-AS1, 1.7E−01, 3.1E−03; |
| | LOC105377355, 1.8E−01, 2.2E−03; LOC107986607, 1.8E−01, 2.2E−03; LOC100131289, 2.0E−01, 8.0E−05; INKA1, 2.0E−01, 5.0E−02; LINC00452, 2.1E−01, |
| | 4.5E−03; LOC105372169, 2.3E−01, 1.6E−04; LILRB5, 2.4E−01, 2.4E−07; LINC00479, 2.5E−01, 3.7E−02; GATA3-AS1, 2.5E−01, 3.2E−02; TP53AIP1, 2.5E−01, |
| | 3.8E−02; HOXA7, 2.5E−01, 4.6E−02; CTSLP2, 2.6E−01, 3.8E−02; LOC51145, 2.6E−01, 2.0E−02; HOXB-AS3, 2.6E−01, 2.6E−03; LINC00964, 2.7E−01, 5.4E−03; |
| | LOC105371313, 2.7E−01, 8.7E−04; LOC105374837, 2.7E−01, 1.1E−02; LOC105369740, 2.7E−01, 2.3E−03; LOC105377096, 2.7E−01, 2.3E−03; |
| | LOC107985138, 2.7E−01, 2.3E−03; LOC107986657, 2.7E−01, 2.3E−03; HOXC9, 2.7E−01, 4.5E−03; FOXL2, 2.8E−01, 3.3E−02; LINC01603, 2.8E−01, 7.2E−03; |
| | CYP2A7, 2.9E−01, 1.6E−02; LOC102725080, 2.9E−01, 2.4E−03; TH, 2.9E−01, 2.4E−03; OLR1, 2.9E−01, 2.3E−07; LOC105374689, 2.9E−01, 1.4E−02; |
| | LOC105369408, 3.0E−01, 4.3E−03; DSG4, 3.0E−01, 3.4E−03; FMR1NB, 3.0E−01, 1.8E−02; LOC105376351, 3.1E−01, 2.7E−02; LOC107984512, 3.1E−01, |
| | 3.5E−02; PCSK6-AS1, 3.1E−01, 8.6E−05; DRAIC, 3.1E−01, 1.7E−02; C15orf32, 3.2E−01, 4.8E−02; LOC105372763, 3.2E−01, 1.5E−03; FLJ32255, 3.2E−01, |
| | 4.9E−02; LOC105374937, 3.3E−01, 4.7E−02; LINC01216, 3.3E−01, 2.0E−03; PGA3, 3.3E−01, 4.5E−04; ZNF556, 3.4E−01, 4.5E−02; LOC102724900, 3.4E−01, |
| | 3.5E−03; LOC105374496, 3.4E−01, 2.6E−02; LOC105372217, 3.4E−01, 2.9E−03; LOC105372104, 3.4E−01, 5.1E−07; HOTAIRM1, 3.5E−01, 8.3E−09; |
| | LOC107985900, 3.6E−01, 1.8E−03; LOC100996263, 3.8E−01, 4.8E−04; LOC107985355, 3.8E−01, 1.5E−07; CLDND2, 3.8E−01, 2.0E−02; LOC102723763, |
| | 3.8E−01, 6.3E−03; LOC101928230, 3.8E−01, 3.5E−02; LOC105369560, 3.9E−01, 3.7E−02; LINC00331, 4.0E−01, 8.8E−06; HOXC10, 4.0E−01, 2.0E−03; |
| | LOC105379099, 4.0E−01, 4.2E−02; LOC105373426, 4.1E−01, 1.2E−09; DBX1, 4.2E−01, 3.4E−02; H4C14, 4.2E−01, 2.4E−03; HCG4B, 4.3E−01, 2.4E−03; |
| | RNF216-IT1, 4.3E−01, 2.0E−02; RSPO3, 4.3E−01, 3.0E−03; CD2, 4.3E−01, 9.1E−17; FETUB, 4.3E−01, 8.5E−05; LOC107986527, 4.4E−01, 4.7E−02; |
| | LOC101927702, 4.4E−01, 1.9E−03; MIR3140, 4.5E−01, 7.7E−03; C9orf106, 4.5E−01, 1.7E−08; LOC107984762, 4.5E−01, 1.7E−08; NEUROG2, 4.5E−01, |
| | 1.3E−08; NUTM2F, 4.5E−01, 7.6E−04; LOC107985854, 4.6E−01, 1.1E−05; TEN1-CDK3, 4.6E−01, 1.3E−08; LOC107986603, 4.6E−01, 1.0E−02; LOC101928943, |
| | 4.6E−01, 2.0E−02; LOC105375544, 4.8E−01, 9.1E−03; LOC107983949, 4.8E−01, 1.8E−05; LOC107984042, 4.8E−01, 4.7E−02; LOC105372749, 4.8E−01, |
| | 4.7E−14; LOC107985847, 5.0E−01, 4.0E−04; MMRN1, 5.0E−01, 3.9E−02; NXF2, 5.0E−01, 5.1E−07; IL33, 5.1E−01, 7.2E−10; PDCD1LG2, 5.1E−01, 1.4E−02; |
| | ZNF571-AS1, 5.2E−01, 8.7E−03; LOC101927888, 5.2E−01, 1.6E−03; FLJ40194, 5.2E−01, 2.9E−06; LOC105373283, 5.3E−01, 1.0E−03; WHAMMP3, 5.4E−01, |
| | 8.1E−03; LOC107984332, 5.4E−01, 1.8E−02; LOC105373345, 5.4E−01, 8.1E−03; GALNT15, 5.4E−01, 6.5E−04; FGF17, 5.5E−01, 8.8E−03; LOC107985770, |
| | 5.5E−01, 3.0E−02; LOC105378310, 5.6E−01, 2.5E−02; LINC00643, 5.7E−01, 1.6E−04; LOC105375369, 5.7E−01, 4.0E−06; LOC101060498, 5.7E−01, 2.3E−02; |
| | LOC101927100, 5.7E−01, 2.6E−02; LOC105370604, 5.7E−01, 3.1E−03; LOC105373636, 5.9E−01, 2.5E−02; LOC105377188, 5.9E−01, 2.6E−03; |
| | LOC107986923, 6.2E−01, 1.1E−04; LOC105378122, 6.2E−01, 2.3E−08; AMIGO2, 6.4E−01, 2.9E−02; SLC23A3, 6.6E−01, 2.2E−03; FERD3L, 6.8E−01, |
| | 1.0E−10; LOC107985934, 6.8E−01, 2.3E−03; OTX1, 6.8E−01, 2.4E−03; LOC107984339, 6.9E−01, 5.2E−05; LINC00448, 6.9E−01, 4.5E−03; LOC105375154, 6.9E−01, |
| | 2.1E−02; JMJD1C−AS1, 6.9E−01, 1.2E−02; ASIC3, 6.9E−01, 1.4E−03; LOC107984818, 6.9E−01, 2.8E−02; DKK1, 6.9E−01, 2.6E−03; SETSIP, 6.9E−01, 2.9E−03; |
| | LOC102724497, 7.0E−01, 1.9E−04; DCAF12L1, 7.2E−01, 1.7E−02; GCNT4, 7.2E−01, 1.5E−07; LOC105373270, 7.2E−01, 1.9E−02; LOC105370774, 7.5E−01, |
| | 2.1E−04; LINC00951, 7.7E−01, 1.4E−02; CDX2, 7.9E−01, 6.7E−06; CYBB, 7.9E−01, 1.3E−07; LOC105376342, 8.0E−01, 5.6E−04; MSX1, 8.0E−01, 5.6E−05; |
| | LINC01500, 8.0E−01, 2.0E−03; LOC105378358, 8.1E−01, 3.1E−02; GATD3A, 8.1E−01, 6.9E−09; LOC105375240, 8.2E−01, 1.6E−02; LOC105372653, 8.5E−01, |
| | 2.0E−05; FOXA1, 8.5E−01, 1.0E−10; LOC105374016, 8.7E−01, 2.6E−03; LOC105374220, 8.8E−01, 3.4E−03; LPL, 9.2E−01, 2.7E−03; FOXA2, 9.2E−01, |
| | 6.5E−10; SSTR1, 9.4E−01, 7.6E−04; ZFP2, 9.4E−01, 9.0E−03; LOC105370302, 9.4E−01, 7.5E−03; CEACAM1, 9.5E−01, 3.9E−02; LOC102724548, 9.6E−01, |
| | 4.4E−02; LOC102723665, 9.8E−01, 4.5E−05; NXPH3, 1.0E+00, 7.4E−03; SIX1, 1.0E+00, 5.8E−09; SPX, 1.0E+00, 1.1E−21; GPR75, 1.1E+00, 2.4E−03; ACTA2, |
| | 1.1E+00, 2.6E−03; GATA3, 1.1E+00, 6.9E−04; GRIN3B, 1.1E+00, 5.3E−03; APLNR, 1.1E+00, 2.6E−03; CER1, 1.1E+00, 5.9E−03; HAND1, 1.1E+00, 4.7E−14; |
| | ZNF205, 1.2E+00, 4.6E−02; LOC105377633, 1.2E+00, 2.4E−12; KLK10, 1.2E+00, 5.8E−03; LOC102724701, 1.2E+00, 1.8E−03; LOC100133985, 1.3E+00, |
| | 8.7E−03; DLK1, 1.3E+00, 3.1E−03; LINC00472, 1.3E+00, 4.8E−03; EOMES, 1.3E+00, 4.2E−17; SPOCK2, 1.3E+00, 1.5E−05; TNFRSF11B, 1.3E+00, 3.8E−02; |
| | LOC399815, 1.3E+00, 1.8E−03; LOC105375969, 1.4E+00, 2.8E−02; RGS14, 1.4E+00, 2.1E−02; LUM, 1.4E+00, 2.0E−14; CCDC92, 1.4E+00, 6.9E−05; SNAI2, |
| | 1.4E+00, 7.6E−05; KIAA0895, 1.4E+00, 1.1E−02; LOC102724419, 1.4E+00, 7.9E−04; LRRC7, 1.5E+00, 3.0E−02; TBX19, 1.5E+00, 4.0E−02; EGFLAM, |
| | 1.6E+00, 2.8E−02; ALPK2, 1.6E+00, 8.2E−03; LOC648987, 1.7E+00, 1.7E−02; ADAMTS9, 1.7E+00, 1.8E−02; BIVM, 1.7E+00, 9.2E−03; GREM1, 1.7E+00, |
| | 1.4E−03; TMCC2, 1.8E+00, 3.7E−02; LOC101929594, 1.9E+00, 3.0E−03; PYGO1, 1.9E+00, 2.3E−03; LOXL4, 1.9E+00, 2.7E−06; ADGRL4, 1.9E+00, 5.0E−08; |
| | CNTN5, 2.0E+00, 4.9E−02; ACTC1, 2.0E+00, 1.6E−02; RABL2B, 2.0E+00, 1.3E−02; SEMA3C, 2.2E+00, 4.2E−04; LRP2, 2.3E+00, 5.0E−03; ZEB1, 2.3E+00, |
| | 3.7E−04; TBXT, 2.5E+00, 1.0E−10; TBC1D9, 2.5E+00, 2.3E−06; MIR99AHG, 2.5E+00, 2.6E−03; MPV17L2, 2.6E+00, 1.4E−02; BMPER, 2.6E+00, 3.7E−03; |
| | DAZAP2, 2.6E+00, 2.8E−02; EDNRB, 2.7E+00, 8.6E−03; PLK2, 2.8E+00, 3.6E−03; DUSP6, 2.8E+00, 3.7E−07; PREX1, 2.8E+00, 3.4E−02; DCC, 2.9E+00, |
| | 1.8E−02; ARL2BP, 3.0E+00, 2.0E−02; EBF2, 3.0E+00, 8.2E−14; PLXNA2, 3.0E+00, 7.6E−06; AFF3, 3.0E+00, 1.5E−02; MN1, 3.0E+00, 2.1E−02; INPP4B, 3.0E+00, |
| | 2.8E−06; COL2A1, 3.1E+00, 4.6E−06; NEDD9, 3.1E+00, 9.8E−03; IGFBP5, 3.1E+00, 1.9E−06; ANTXR2, 3.1E+00, 2.9E−03; LRIG3, 3.2E+00, 4.9E−02; CDH10, |
| | 3.3E+00, 5.7E−14; DOK6, 3.4E+00, 2.0E−02; MMP2, 3.4E+00, 1.1E−04; FST, 3.4E+00, 3.5E−02; KIF26B, 3.5E+00, 1.4E−02; ITGB8, 3.5E+00, 4.2E−02; |
| | HSPA2, 3.6E+00, 1.5E−03; ZNHIT3, 3.6E+00, 2.6E−02; ACAD8, 3.6E+00, 2.1E−02; H2BC11, 3.7E+00, 4.0E−02; H4C1, 3.8E+00, 3.8E−02; ANKRD1, 3.8E+00, |
| | 4.4E−12; S100A11, 3.8E+00, 3.9E−02; PARVA, 3.9E+00, 3.4E−02; ANXA1, 4.0E+00, 1.0E−07; ZNF197, 4.0E+00, 3.4E−02; LOXL2, 4.0E+00, 7.6E−04; |
| | CRABP2, 4.2E+00, 3.8E−02; SH3BGRL3, 4.3E+00, 4.2E−02; WLS, 4.3E+00, 3.6E−14; RHOBTB3, 4.4E+00, 1.5E−07; NEXN, 4.4E+00, 1.2E−02; ZEB2, 4.5E+00, |
| | 5.7E−13; MACROH2A2, 4.5E+00, 2.4E−04; H2BC21, 4.6E+00, 2.3E−03; FAM32A, 4.7E+00, 1.9E−02; LINC00458, 4.7E+00, 8.3E−09; SLC50A1, 4.9E+00, |
| | 2.2E−05; LHFP, 4.9E+00, 1.6E−02; GPX8, 4.9E+00, 2.1E−02; FAM120B, 5.2E+00, 2.9E−02; PTPRM, 5.3E+00, 2.4E−02; MRPL52, 5.3E+00, 4.1E−02; TAF9B, |
| | 5.4E+00, 2.3E−03; PPFIBP1, 5.5E+00, 3.9E−02; H2BC17, 5.9E+00, 4.1E−05; FLRT2, 5.9E+00, 1.7E−03; SAT1, 6.0E+00, 5.8E−07; CTSV, 6.0E+00, 1.5E−02; |
| | TCEAL4, 6.1E+00, 2.7E−02; COL5A2, 6.2E+00, 3.3E−09; PKNOX2, 6.2E+00, 1.6E−07; NDUFB2, 6.4E+00, 2.4E−03; CDK6, 6.6E+00, 3.9E−02; NNAT, 6.8E+00, |
| | 1.9E−06; CD63, 7.3E+00, 3.3E−02; H3C10, 7.4E+00, 2.3E−03; ACTB, 7.6E+00, 4.3E−05; H2AC20, 8.0E+00, 3.2E−02; FSTL1, 8.4E+00, 7.5E−03; H2AC13, |
| | 8.4E+00, 8.3E−05; SEPTIN11, 8.4E+00, 3.6E−03; H4C12, 8.5E+00, 4.1E−10; VIM, 8.6E+00, 8.1E−06; GREB1L, 8.7E+00, 1.0E−06; H3C8, 8.7E+00, 1.9E−06; |
| | H2BC4, 8.7E+00, 2.0E−03; SERPINE2, 8.8E+00, 2.3E−07; H1-2, 8.8E+00, 3.2E−02; H2BC9, 8.8E+00, 1.6E−03; H2AC12, 9.0E+00, 1.5E−04; H4C3, 9.0E+00, |
| | 1.9E−02; CNTNAP2, 9.1E+00, 2.9E−03; FBN2, 9.2E+00, 4.7E−05; HAPLN1, 9.5E+00, 2.3E−26; H2AC8, 9.6E+00, 4.7E−04; HMGN1, 9.7E+00, 2.3E−03; |
| | EFNA5, 1.0E+01, 1.3E−03; H2AC11, 1.0E+01, 2.3E−04; HMGN2, 1.0E+01, 2.7E−04; NR6A1, 1.0E+01, 6.9E−04; H2AC17, 1.1E+01, 1.2E−03; NTS, 1.1E+01, |
| | 2.5E−16; H3C2, 1.1E+01, 5.6E−04; H2AC14, 1.2E+01, 1.5E−06; ID3, 1.3E+01, 4.3E−14; SLIT2, 1.3E+01, 3.8E−14; H4C5, 1.4E+01, 3.0E−08; RN7SK, 1.8E+01, |
| | 4.4E−12; PRTG, 1.8E+01, 2.7E−49 |
| 2 | LINC00428, −3.9E+01, 8.8E−23; ESRG, −3.3E+01, 2.2E−18; MME, −2.8E+01, 2.1E−09; L1TD1, −2.6E+01, 1.2E−14; GJA1, −2.5E+01, 5.8E−08; PTMA, −2.4E+01, |
| | 2.1E−20; RPSA, −2.4E+01, 2.1E−07; LOC105375710, −2.3E+01, 1.6E−14; GSTP1, −2.3E+01, 7.6E−07; PRDX1, −2.3E+01, 9.0E−07; DNMT3B, −2.1E+01, 7.1E−12; |
| | POU5F1, −2.1E+01, 9.8E−06; UQCRH, −2.1E+01, 2.6E−06; RPL8, −2.1E+01, 6.9E−06; SKIL, −2.0E+01, 2.4E−05; RPS6, −2.0E+01, 3.4E−10; RPL14, |
| | −2.0E+01, 1.3E−05; LRFN5, −2.0E+01, 4.2E−06; LOC101927668, −2.0E+01, 1.1E−03; RPL10A, −2.0E+01, 1.7E−05; RPL34, −2.0E+01, 2.5E−05; HSPD1, |
| | −1.9E+01, 1.0E−06; RPS8, −1.9E+01, 9.0E−08; RPL15, −1.9E+01, 4.0E−05; DPYSL3, −1.9E+01, 5.5E−05; HSP90AB1, −1.9E+01, 2.3E−10; RPL3, −1.9E+01, 2.6E−06; |
| | HSPA9, −1.9E+01, 6.9E−05; RPL13, −1.9E+01, 3.3E−05; RPL13A, −1.9E+01, 1.3E−07; LINC01194, −1.9E+01, 1.0E−03; CSDE1, −1.9E+01, 5.5E−05; RACK1, |
| | −1.8E+01, 2.0E−04; RPS3, −1.8E+01, 1.1E−04; SGCD, −1.8E+01, 4.8E−04; DPPA4, −1.8E+01, 2.2E−05; RPS24, −1.8E+01, 9.4E−05; HSP90AA1, −1.8E+01, 8.8E−09; |
| | RPS27A, −1.8E+01, 1.9E−04; RPL9, −1.7E+01, 6.6E−04; EEF1A1, −1.7E+01, 5.6E−09; H4C3, −1.7E+01, 4.8E−03; SPG20, −1.7E+01, 7.6E−05; RPL29, |
| | −1.7E+01, 9.6E−04; RPL11, −1.7E+01, 9.1E−05; NPM1, −1.7E+01, 5.4E−07; EEF1G, −1.7E+01, 2.4E−03; CCT3, −1.7E+01, 1.1E−03; SERBP1, −1.7E+01, 4.1E−04; |
| | GAPDH, −1.6E+01, 1.7E−05; HSPA8, −1.6E+01, 3.6E−04; RPS5, −1.6E+01, 2.1E−03; RPL23, −1.6E+01, 1.1E−03; RPS2, −1.6E+01, 2.4E−03; RPL5, −1.6E+01, |
| | 1.2E−03; FTH1, −1.6E+01, 3.2E−03; RPLPO, −1.6E+01, 2.6E−03; RPLP1, −1.6E+01, 1.3E−04; RIF1, −1.6E+01, 1.5E−03; H2AZ1, −1.6E+01, 4.4E−03; PKM, |
| | −1.6E+01, 4.6E−03; RPS15A, −1.5E+01, 5.1E−03; PABPC1, −1.5E+01, 3.2E−04; RPL37A, −1.5E+01, 5.4E−03; ADH5, −1.5E+01, 2.4E−03; CBX3, −1.5E+01, 3.9E−03; |
| | C12orf60, −1.5E+01, 1.3E−04; TUBA1B, −1.5E+01, 7.3E−03; RBPJ, −1.5E+01, 5.4E−03; RPS23, −1.5E+01, 6.3E−03; RPL26, −1.5E+01, 8.9E−03; RPS7, |
| | −1.5E+01, 8.5E−03; UBB, −1.5E+01, 9.1E−03; RPL18, −1.5E+01, 9.1E−03; PARP1, −1.5E+01, 4.5E−03; LIN28A, −1.5E+01, 2.6E−04; RPL19, −1.5E+01, 5.5E−03; |
| | TDGF1, −1.5E+01, 4.5E−03; IDO1, −1.4E+01, 2.8E−03; RPS18, −1.4E+01, 2.8E−03; RPS11, −1.4E+01, 1.5E−02; TERF1, −1.4E+01, 6.9E−03; UGP2, −1.4E+01, |
| | 1.4E−03; PA2G4, −1.4E+01, 6.3E−03; HSPA5, −1.4E+01, 2.3E−02; LOC101929194, −1.4E+01, 2.6E−02; POLR3G, −1.4E+01, 1.5E−02; H1-5, −1.4E+01, 4.5E−02; |
| | RPL10, −1.4E+01, 2.5E−02; SRSF3, −1.4E+01, 2.3E−02; LDHB, −1.4E+01, 1.4E−02; SERINC5, −1.4E+01, 2.3E−02; RPL35A, −1.3E+01, 2.6E−02; ACTB, |
| | −1.3E+01, 3.4E−04; RPS20, −1.3E+01, 3.1E−02; XPO1, −1.3E+01, 9.8E−03; RPL12, −1.3E+01, 2.9E−02; HNRNPA2B1, −1.3E+01, 9.7E−05; RPS12, −1.3E+01, |
| | 1.9E−02; EIF5B, −1.3E+01, 3.1E−02; NCL, −1.3E+01, 1.1E−03; PAPOLA, −1.3E+01, 3.1E−02; CEBPZ, −1.3E+01, 3.0E−02; RPS28, −1.3E+01, 2.3E−02; RPL4, |
| | −1.3E+01, 4.7E−03; RPL7, −1.3E+01, 3.9E−02; TPM3, −1.3E+01, 2.9E−02; RPS3A, −1.3E+01, 4.0E−02; RPL6, −1.3E+01, 2.5E−02; PEBP1, −1.3E+01, 2.6E−02; |
| | KRT8, −1.3E+01, 4.4E−03; MLEC, −1.3E+01, 2.6E−02; HNRNPK, −1.3E+01, 3.9E−02; THRAP3, −1.3E+01, 4.5E−02; COX7C, −1.3E+01, 3.2E−02; IFITM1, |
| | −1.3E+01, 5.6E−04; HSPA4, −1.3E+01, 4.6E−02; EIF4B, −1.3E+01, 4.5E−02; CD24, −1.3E+01, 6.6E−03; CANX, −1.3E+01, 2.9E−02; EID1, −1.2E+01, 2.5E−02; |
| | G3BP2, −1.2E+01, 4.5E−02; PCBP1, −1.2E+01, 4.5E−03; RPL31, −1.2E+01, 3.7E−02; ATP5PD, −1.2E+01, 1.5E−02; RPS26, −1.2E+01, 2.7E−02; HELLS, |
| | −1.2E+01, 2.4E−02; SET, −1.2E+01, 3.3E−02; MCM6, −1.2E+01, 4.2E−02; ARMT1, −1.2E+01, 1.2E−02; NEFL, −1.1E+01, 5.5E−05; PSMD12, −1.1E+01, 4.3E−02; |
| | AARS1, −1.1E+01, 4.6E−02; SHFM1, −1.1E+01, 3.9E−02; GALNT3, −1.1E+01, 2.1E−02; H4C5, −1.1E+01, 4.7E−02; TXLNG, −1.1E+01, 2.9E−02; TIMELESS, |
| | −1.0E+01, 2.1E−02; OIP5-AS1, −1.0E+01, 4.2E−02; NOL7, −9.8E+00, 4.2E−02; GAL, −9.7E+00, 4.2E−05; SLC25A5, −9.5E+00, 4.3E−02; TBC1D23, −8.8E+00, |
| | 4.5E−02; HTR2C, −8.6E+00, 2.9E−02; NME1, −7.8E+00, 2.3E−02; NDUFAF4, −6.8E+00, 2.4E−02; H2BC6, −6.7E+00, 3.1E−02; LOC107986623, −6.0E+00, |
| | 1.8E−02; HHLA1, −5.9E+00, 1.6E−03; ABHD12B, −5.4E+00, 2.6E−03; LOC107987087, −4.5E+00, 2.4E−02; LOC105373409, −4.0E+00, 4.3E−02; LINC01508, |
| | −3.4E+00, 4.9E−03; LOC107984606, 3.2E−01, 2.7E−02; MIR5190, 3.2E−01, 2.7E−02; LOC105374201, 3.3E−01, 2.6E−02; LOC107985997, 3.8E−01, 3.0E−02; |
| | R3HDML, 4.7E−01, 5.1E−03; CNGA2, 4.8E−01, 1.6E−03; CSF2RB, 4.8E−01, 1.6E−03; LOC105370739, 4.8E−01, 1.6E−03; BTF3P11, 5.0E−01, 5.8E−08; |
| | LOC101929660, 5.0E−01, 5.8E−08; LOC101930496, 5.0E−01, 5.8E−08; LOC107983993, 5.0E−01, 5.8E−08; LOC105373417, 5.5E−01, 6.2E−05; GBP2, 5.7E−01, |
| | 1.4E−03; H2BP1, 5.7E−01, 1.4E−03; LOC107984590, 5.7E−01, 2.4E−02; AKR1C4, 5.8E−01, 4.1E−04; STRA8, 6.9E−01, 1.0E−03; LOC84214, 7.1E−01, 2.6E−04; |
| | KBTBD11-OT1, 7.2E−01, 5.8E−03; PTGER1, 7.4E−01, 5.6E−03; LOC105373654, 7.5E−01, 2.8E−02; CPB2, 7.5E−01, 6.4E−03; HTN1, 7.8E−01, 1.4E−03; |
| | SLC26A1, 7.8E−01, 2.9E−02; GOS2, 7.9E−01, 4.6E−03; HLA-H, 8.0E−01, 1.3E−05; LCE1A, 8.0E−01, 1.3E−05; LINC00377, 8.0E−01, 1.3E−05; LOC400627, 8.2E−01, |
| | 2.5E−02; LOC105371532, 8.3E−01, 1.0E−03; LOC105372088, 8.4E−01, 3.4E−02; ITGB2, 8.5E−01, 3.0E−03; LOC105369734, 8.7E−01, 1.6E−04; |
| | LOC105374595, 8.8E−01, 3.2E−02; CRTAM, 9.0E−01, 1.0E−02; LOC101929951, 9.1E−01, 1.1E−02; SLC22A8, 9.1E−01, 2.6E−03; LOC107987020, 9.2E−01, |
| | 3.4E−02; LOC107984512, 9.3E−01, 1.1E−02; LOC105377477, 9.8E−01, 1.1E−02; NR2F2, 9.9E−01, 5.0E−02; LOC107984502, 1.0E+00, 1.1E−06; COL3A1, |
| | 1.1E+00, 1.4E−02; LINC01590, 1.1E+00, 1.4E−03; TGM1, 1.1E+00, 2.6E−02; LOC105376345, 1.1E+00, 6.2E−05; LOC107986263, 1.2E+00, 8.8E−09; |
| | LINC01135, 1.2E+00, 8.2E−03; DIAPH3-AS1, 1.2E+00, 4.8E−03; LOC105375130, 1.3E+00, 1.3E−02; LOC102723468, 1.4E+00, 2.1E−02; TFF3, 1.4E+00, |
| | 4.7E−02; LOC105378480, 1.5E+00, 2.3E−05; LOC105374709, 1.5E+00, 1.6E−02; GATA3, 1.6E+00, 1.7E−02; LOC105369745, 1.8E+00, 3.9E−03; MTHFS, |
| | 2.0E+00, 7.9E−03; LOXL4, 2.0E+00, 3.2E−02; CALHM2, 2.0E+00, 1.7E−02; ANKRD19P, 2.1E+00, 4.6E−02; DEFB1, 2.1E+00, 1.4E−10; LOC729603, 2.2E+00, |
| | 4.7E−02; PCDHGB6, 2.2E+00, 3.2E−02; LOC107983979, 2.2E+00, 2.4E−02; LOC105370534, 2.2E+00, 3.8E−23; COL11A2, 2.2E+00, 8.0E−04; MIR186, |
| | 2.3E+00, 1.9E−10; COX7B2, 2.3E+00, 1.8E−06; LOC101929584, 2.3E+00, 1.6E−14; SSC5D, 2.3E+00, 4.3E−02; LOC105371092, 2.4E+00, 1.0E−04; |
| | LOC101928509, 2.4E+00, 8.1E−11; PLA2G15, 2.4E+00, 1.8E−03; PCDH20, 2.4E+00, 1.9E−10; TNMD, 2.4E+00, 1.1E−02; LINC00877, 2.4E+00, 1.5E−03; |
| | BC01, 2.4E+00, 2.1E−14; LOC101927701, 2.5E+00, 1.5E−83; LOC102725227, 2.5E+00, 7.7E−11; UGT2B15, 2.5E+00, 5.3E−82; PHEX, 2.5E+00, 4.8E−03; |
| | HSD17B2, 2.5E+00, 1.1E−02; FGA, 2.5E+00, 5.1E−155; IL17F, 2.5E+00, 5.1E−155; PRADC1, 2.5E+00, 2.4E−05; HNF1B, 2.5E+00, 2.7E−09; LOC102723557, |
| | 2.5E+00, 2.4E−02; NYAP1, 2.5E+00, 1.2E−03; SNORD45B, 2.5E+00, 2.2E−10; LOC107985275, 2.6E+00, 1.8E−02; LINC01600, 2.6E+00, 2.9E−06; MIR579, |
| | 2.6E+00, 1.6E−92; HSD11B2, 2.6E+00, 1.6E−02; HIGD1B, 2.6E+00, 3.6E−15; LOC105377596, 2.7E+00, 5.8E−03; AGAP5, 2.7E+00, 3.1E−02; ANKRD1, |
| | 2.7E+00, 1.3E−02; LOC105378780, 2.7E+00, 1.2E−46; MGAM, 2.7E+00, 2.1E−10; LOC101929007, 2.7E+00, 1.7E−02; CDC37L1-AS1, 2.7E+00, 3.5E−14; |
| | PARP12, 2.8E+00, 4.4E−03; RIMBP2, 2.8E+00, 2.4E−02; SPDYE16, 2.8E+00, 2.0E−47; HOGA1, 2.9E+00, 1.7E−04; LOC105376287, 2.9E+00, 3.6E−03; |
| | LOC105370532, 2.9E+00, 3.9E−03; LOC105377100, 2.9E+00, 4.0E−16; MYLK4, 2.9E+00, 4.5E−09; KRT7, 2.9E+00, 2.6E−02; S100A6, 2.9E+00, 2.0E−03; |
| | ZEB1, 2.9E+00, 2.7E−02; LOC107986431, 2.9E+00, 3.6E−08; LOC107985343, 3.0E+00, 2.5E−02; STEAP1, 3.0E+00, 2.3E−02; CARD10, 3.0E+00, 2.0E−04; |
| | LOC107987122, 3.0E+00, 4.6E−04; OGN, 3.0E+00, 2.0E−02; LOC105375846, 3.1E+00, 2.3E−29; FANK1, 3.2E+00, 4.3E−02; ARHGAP25, 3.2E+00, 3.0E−02; |
| | SHISA2, 3.2E+00, 2.6E−02; SAP30, 3.2E+00, 2.3E−04; NKAPP1, 3.2E+00, 2.4E−02; DNAJC25, 3.2E+00, 1.3E−02; ZNF446, 3.2E+00, 5.1E−04; |
| | LOC105373146, 3.3E+00, 4.6E−03; PCDH19, 3.3E+00, 8.7E−06; LOC105371416, 3.4E+00, 2.7E−08; NELL1, 3.4E+00, 3.6E−02; APOB, 3.5E+00, 2.2E−08; |
| | SOX6, 3.6E+00, 2.9E−03; TMEM175, 3.6E+00, 9.0E−05; C1orf216, 3.7E+00, 8.6E−03; TRIM54, 3.7E+00, 2.4E−03; PWRN1, 3.7E+00, 4.5E−04; FGF17, |
| | 3.8E+00, 2.3E−70; RADIL, 3.8E+00, 2.6E−02; CYB5D2, 3.9E+00, 1.4E−02; LOC105378071, 4.0E+00, 1.2E−04; SLC8B1, 4.0E+00, 4.3E−02; USP51, 4.1E+00, |
| | 4.2E−02; PUS10, 4.3E+00, 4.2E−02; HPSE2, 4.3E+00, 1.9E−02; RFXANK, 4.3E+00, 2.9E−02; LOC102724810, 4.4E+00, 3.5E−02; GALNT16, 4.5E+00, 4.9E−02; |
| | IPP, 4.5E+00, 3.0E−02; ATP1A3, 4.7E+00, 1.2E−02; SYTL5, 4.7E+00, 1.0E−02; PAX7, 4.9E+00, 1.3E−04; LRTOMT, 5.1E+00, 3.9E−03; SULF1, 5.1E+00, |
| | 3.4E−03; GLIS3, 5.2E+00, 1.2E−06; SLC39A8, 5.2E+00, 2.9E−03; ZEB2, 5.6E+00, 7.3E−07; ARNTL, 5.7E+00, 1.1E−02; LINC01089, 5.7E+00, 3.3E−02; |
| | LRP1B, 5.8E+00, 2.4E−02; DENND1B, 5.9E+00, 4.3E−02; SF3A2, 6.0E+00, 2.6E−02; LOC102724623, 6.0E+00, 6.8E−03; UNC5C, 6.1E+00, 1.1E−03; RNPC3, |
| | 6.2E+00, 3.4E−02; PLEKHG1, 6.2E+00, 2.4E−02; KCNIP4-IT1, 6.2E+00, 3.5E−04; DPYD, 6.4E+00, 6.9E−07; MEGF6, 6.6E+00, 8.4E−03; LOC107984805, |
| | 6.6E+00, 3.1E−02; DSCAML1, 6.6E+00, 4.1E−02; BCL7A, 6.6E+00, 1.9E−02; RNLS, 7.2E+00, 2.4E−02; PKNOX2, 7.2E+00, 1.8E−03; CITED2, 7.2E+00, 3.3E−02; |
| | DCC, 7.2E+00, 2.0E−04; ZFHX4, 7.4E+00, 1.7E−05; SAMD4A, 7.6E+00, 2.5E−02; SH3YL1, 7.6E+00, 8.5E−03; TBC1D19, 8.0E+00, 3.3E−02; SIL1, |
| | 8.0E+00, 3.5E−03; BMPER, 8.0E+00, 6.5E−05; TLE3, 8.0E+00, 4.3E−02; ARID5B, 8.0E+00, 3.3E−02; BMPR1B, 8.2E+00, 4.8E−03; OSBPL3, 8.5E+00, 2.5E−02; |
| | LOC105378308, 8.5E+00, 1.4E−05; PCDH15, 8.7E+00, 4.4E−03; GRIK2, 8.9E+00, 1.1E−02; LINC00278, 8.9E+00, 2.0E−10; EFNB2, 9.0E+00, 1.4E−04; |
| | EPHA4, 9.1E+00, 2.5E−02; ID4, 9.3E+00, 4.8E−03; CDH2, 9.3E+00, 2.7E−02; IKZF2, 9.3E+00, 2.5E−02; CDH11, 9.5E+00, 2.1E−03; TANC2, 9.5E+00, |
| | 1.6E−02; STXBP5L, 9.7E+00, 2.5E−02; SRGAP1, 9.7E+00, 1.3E−02; EPHB1, 9.7E+00, 8.9E−04; HDAC9, 9.8E+00, 4.3E−02; FRY, 9.8E+00, 1.8E−02; PLAG1, |
| | 9.9E+00, 2.4E−03; IGSF11, 9.9E+00, 1.9E−02; COL4A6, 1.0E+01, 1.9E−02; CDH6, 1.0E+01, 1.1E−03; TTC3, 1.0E+01, 3.6E−02; CADM2, 1.0E+01, 3.8E−02; |
| | DAAM1, 1.0E+01, 3.6E−02; LOC102723568, 1.0E+01, 4.7E−02; AHNAK, 1.0E+01, 1.8E−03; RIMS2, 1.1E+01, 2.9E−02; WIPF2, 1.1E+01, 2.6E−03; ABCA1, |
| | 1.1E+01, 4.2E−02; SNX25, 1.1E+01, 4.3E−02; MLLT3, 1.1E+01, 1.5E−03; MYOF, 1.1E+01, 5.3E−04; MTCL1, 1.1E+01, 2.3E−02; MEMO1, 1.1E+01, 2.3E−02; |
| | ASPH, 1.1E+01, 2.9E−02; NTRK3, 1.1E+01, 8.1E−03; CDK8, 1.1E+01, 4.7E−02; PTPRM, 1.1E+01, 1.6E−03; CNTN5, 1.1E+01, 9.2E−06; UNC5D, 1.2E+01, |
| | 2.3E−02; SORBS2, 1.2E+01, 2.3E−02; PPP2R3A, 1.2E+01, 4.1E−02; RELCH, 1.2E+01, 1.8E−02; DIP2C, 1.2E+01, 3.0E−02; DST, 1.2E+01, 2.6E−02; CADPS, |
| | 1.2E+01, 2.5E−02; DYNC2H1, 1.2E+01, 4.7E−02; XKR4, 1.2E+01, 6.8E−03; ZNF407, 1.2E+01, 8.8E−03; CTDSPL2, 1.2E+01, 1.4E−02; CDK14, 1.2E+01, |
| | 1.6E−03; HAPLN1, 1.2E+01, 6.9E−05; LMO3, 1.2E+01, 4.0E−07; ARHGAP10, 1.2E+01, 4.1E−04; CHKA, 1.2E+01, 2.8E−02; MAPK8, 1.2E+01, 4.0E−02; GTDC1, |
| | 1.2E+01, 1.2E−02; GLI3, 1.3E+01, 1.8E−03; DENND1A, 1.3E+01, 2.3E−02; PBX1, 1.3E+01, 2.8E−04; GPATCH2, 1.3E+01, 1.1E−02; TBCK, 1.3E+01, 2.2E−02; |
| | ALCAM, 1.3E+01, 2.6E−02; ARID1B, 1.3E+01, 1.6E−02; ANKDD1A, 1.3E+01, 3.4E−03; C8orf34, 1.3E+01, 2.1E−03; LOC100288798, 1.3E+01, 8.3E−03; |
| | MAGI1, 1.3E+01, 8.7E−03; SOX5, 1.3E+01, 2.6E−02; PALLD, 1.3E+01, 2.0E−03; RBM26, 1.3E+01, 3.0E−02; THRB, 1.3E+01, 8.7E−03; BASP1, 1.3E+01, |
| | 1.1E−02; TCF7L2, 1.3E+01, 2.4E−03; TOX, 1.3E+01, 2.1E−02; CDYL, 1.3E+01, 4.7E−02; BCKDHB, 1.3E+01, 3.9E−02; PGAP1, 1.3E+01, 6.8E−03; MNAT1, 1.3E+01, |
| | 2.6E−02; BCAS3, 1.3E+01, 2.7E−02; ZNF521, 1.3E+01, 1.5E−03; NLGN4Y, 1.3E+01, 4.9E−02; ADNP, 1.3E+01, 2.7E−02; PPP1R9A, 1.3E+01, 2.8E−02; PLD5, |
| | 1.3E+01, 6.9E−03; FRAS1, 1.3E+01, 1.0E−02; PTPRJ, 1.3E+01, 5.6E−03; ROBO1, 1.4E+01, 2.9E−02; FIGN, 1.4E+01, 2.5E−02; CHD7, 1.4E+01, 1.2E−02; |
| | KCNQ10T1, 1.4E+01, 3.3E−02; DCBLD2, 1.4E+01, 8.4E−03; KMT2C, 1.4E+01, 2.5E−02; KCNN2, 1.4E+01, 1.9E−03; SIPA1L1, 1.4E+01, 2.3E−02; DPP10, |
| | 1.4E+01, 2.1E−02; KCNT2, 1.4E+01, 2.4E−02; ITGB8, 1.4E+01, 8.0E−04; NEBL, 1.4E+01, 5.5E−04; CTNND2, 1.4E+01, 2.2E−02; CHD9, 1.4E+01, 1.6E−02; |
| | RABGAP1, 1.4E+01, 6.9E−03; AKAP13, 1.4E+01, 1.3E−02; ZNF608, 1.4E+01, 8.4E−03; UBE2E2, 1.4E+01, 1.5E−02; XKR6, 1.4E+01, 4.5E−03; COL4A5, |
| | 1.5E+01, 1.1E−02; CDH12, 1.5E+01, 4.0E−06; NRIP1, 1.5E+01, 6.9E−07; EPB41L4A, 1.5E+01, 1.8E−04; ZFAND3, 1.5E+01, 1.1E−02; MCC, 1.5E+01, |
| | 1.8E−03; TENM4, 1.5E+01, 2.4E−03; LOC107986770, 1.5E+01, 8.4E−03; ADGRB3, 1.5E+01, 2.5E−03; SMYD3, 1.5E+01, 8.1E−03; PRIM2, 1.5E+01, 6.8E−03; |
| | FBN2, 1.5E+01, 3.3E−09; FHIT, 1.5E+01, 8.4E−03; TENM2, 1.5E+01, 4.4E−03; EFNA5, 1.5E+01, 6.3E−03; NCOA1, 1.5E+01, 6.6E−03; NEAT1, 1.5E+01, |
| | 1.2E−02; COBL, 1.6E+01, 3.9E−03; COL11A1, 1.6E+01, 3.9E−03; NFIB, 1.6E+01, 3.0E−03; TRPM3, 1.6E+01, 5.8E−03; MALAT1, 1.6E+01, 3.1E−15; MBD5, |
| | 1.6E+01, 4.0E−03; RBFOX1, 1.6E+01, 3.5E−03; GRIP1, 1.6E+01, 9.6E−04; TBL1X, 1.7E+01, 8.6E−04; CMIP, 1.7E+01, 1.2E−03; CBLB, 1.7E+01, 3.9E−04; |
| | CADPS2, 1.7E+01, 1.0E−03; ST6GALNAC5, 1.7E+01, 3.3E−05; ZC3H12B, 1.7E+01, 1.4E−03; FBXL17, 1.7E+01, 3.8E−05; CACNA2D1, 1.7E+01, 1.2E−03; |
| | NECTIN3, 1.7E+01, 6.3E−04; MAPK10, 1.8E+01, 1.7E−05; RNF130, 1.8E+01, 1.7E−04; MAGI2, 1.8E+01, 2.4E−04; NRCAM, 1.9E+01, 3.2E−06; PRTG, |
| | 1.9E+01, 9.8E−22; LOC102724392, 1.9E+01, 8.2E−11; LOC101928437, 1.9E+01, 4.2E−08; GREB1L, 2.0E+01, 2.7E−11; KLF12, 2.0E+01, 3.8E−05; CNTN4, |
| | 2.0E+01, 2.7E−05; MACROD2, 2.0E+01, 3.0E−05; DACH1, 2.0E+01, 1.3E−09; PCDH7, 2.0E+01, 3.8E−05; WWOX, 2.1E+01, 6.0E−06; PTPRD, 2.1E+01, |
| | 1.2E−05; PRKG1, 2.1E+01, 2.1E−05; ERBB4, 2.2E+01, 7.5E−10; SSBP2, 2.2E+01, 2.1E−06; SOX2-OT, 2.2E+01, 5.0E−06; NRXN3, 2.4E+01, 3.9E−07; CDH9, |
| | 2.4E+01, 1.5E−09; CTNNA2, 3.6E+01, 2.7E−15; IL1RAPL1, 3.7E+01, 1.7E−12; KCNIP4, 5.4E+01, 5.7E−24; |
| 1 | SHISA9, −1.8E+01, 1.9E−24; MAGI2, −1.4E+01, 4.5E−15; GRID2, −1.4E+01, 2.2E−63; CD24, −1.3E+01, 4.3E−21; TOX, −1.3E+01, 5.3E−14; MGAT4C, −1.3E+01, |
| | 2.4E−13; FOXO1, −1.2E+01, 1.2E−11; PLPP1, −1.1E+01, 5.7E−11; ESRG, −1.1E+01, 7.1E−11; ANKRD18CP, −1.1E+01, 3.7E−10; AASS, −1.1E+01, 2.2E−13; |
| | DNMT3B, −1.1E+01, 1.3E−13; BNC2, −1.0E+01, 1.7E−09; SORBS2, −1.0E+01, 2.4E−09; NPFFR2, −1.0E+01, 3.2E−30; FLT1, −1.0E+01, 3.7E−10; AP1S2, |
| | −9.9E+00, 1.4E−08; UGP2, −9.6E+00, 3.3E−09; POU5F1, −9.5E+00, 3.7E−08; USP44, −9.5E+00, 2.6E−08; USP9X, −9.5E+00, 7.9E−14; CDH1, −9.5E+00, 3.9E−08; |
| | LINC01194, −9.4E+00, 2.4E−08; PODXL, −9.2E+00, 1.0E−08; LOC107986777, −9.0E+00, 2.0E−08; KIAA0825, −9.0E+00, 4.5E−06; PLS3, −8.8E+00, |
| | 1.4E−06; JARID2, −8.4E+00, 2.3E−08; SYN3, −8.3E+00, 2.0E−06; POLR3G, −8.1E+00, 8.1E−07; CSMD2, −7.9E+00, 2.4E−05; RABGAP1L, −7.6E+00, 3.4E−06; |
| | GALNT17, −7.6E+00, 4.8E−05; RPS2, −7.5E+00, 1.2E−04; DAB1, −7.4E+00, 7.8E−05; LDB2, −7.4E+00, 1.2E−04; ESRP1, −7.3E+00, 3.9E−05; CNTN1, −7.1E+00, |
| | 9.4E−08; GNPTAB, −7.0E+00, 1.3E−05; OSBPL10, −7.0E+00, 1.8E−04; DPPA4, −7.0E+00, 2.2E−04; LMO3, −6.9E+00, 8.9E−09; GABRB3, −6.7E+00, 6.9E−04; |
| | MFGE8, −6.7E+00, 8.7E−04; LIN28A, −6.7E+00, 2.2E−06; LOC101929194, −6.7E+00, 5.6E−06; FAU, −6.6E+00, 7.4E−04; L1TD1, −6.6E+00, 2.6E−05; RBPJ, |
| | −6.5E+00, 1.3E−03; DPYSL3, −6.3E+00, 2.2E−03; TARS1, −6.3E+00, 2.4E−03; PTMA, −6.2E+00, 3.9E−08; LOC107985661, −6.2E+00, 6.4E−05; EEF1A1, |
| | −6.2E+00, 2.9E−09; APELA, −6.2E+00, 2.6E−03; RPS11, −6.0E+00, 5.3E−03; TERF1, −6.0E+00, 1.7E−03; FGF12, −5.9E+00, 3.6E−04; ND4, −5.9E+00, 3.1E−19; |
| | SERBP1, −5.8E+00, 2.0E−03; NLGN4X, −5.8E+00, 1.2E−02; LOC107986324, −5.8E+00, 1.7E−07; SLC24A2, −5.7E+00, 4.3E−04; EZR, −5.6E+00, 5.5E−03; |
| | USO1, −5.5E+00, 1.1E−02; CTSC, −5.5E+00, 9.2E−03; ND3, −5.5E+00, 9.3E−03; RPL15, −5.5E+00, 9.4E−03; SEPHS1, −5.5E+00, 6.8E−03; CGNL1, −5.4E+00, |
| | 4.7E−03; LOC729732, −5.4E+00, 1.7E−02; RPS8, −5.4E+00, 4.7E−04; SLC16A1, −5.4E+00, 1.8E−02; DIAPH2, −5.4E+00, 2.3E−02; RPL13A, −5.4E+00, 5.7E−04; |
| | TRIM71, −5.4E+00, 2.0E−03; RPL18, −5.3E+00, 1.5E−02; VWDE, −5.3E+00, 5.9E−04; NR3C1, −5.3E+00, 3.4E−06; PHF21B, −5.3E+00, 2.2E−02; LRRTM4, |
| | −5.3E+00, 3.2E−02; NPM1, −5.3E+00, 2.0E−04; IDO1, −5.2E+00, 1.0E−04; RPL12, −5.2E+00, 1.9E−02; TSSC2, −5.2E+00, 7.2E−03; PRKCA, −5.2E+00, 1.3E−02; |
| | GPR176, −5.2E+00, 2.5E−02; TMEM132D, −5.1E+00, 4.9E−02; ND4L, −5.1E+00, 4.1E−02; CDH9, −5.1E+00, 8.4E−03; HSPD1, −5.0E+00, 1.0E−02; RPSA, |
| | −5.0E+00, 4.4E−02; RPS7, −5.0E+00, 3.8E−02; TFRC, −5.0E+00, 5.7E−03; TNRC6A, −5.0E+00, 3.0E−02; COX3, −4.9E+00, 2.3E−06; ADGRV1, −4.9E+00, 4.1E−02; |
| | RRAS2, −4.9E+00, 3.4E−02; RPS27A, −4.9E+00, 3.4E−02; IGF2BP3, −4.8E+00, 2.3E−02; CYTB, −4.8E+00, 1.0E−04; ZNF770, −4.8E+00, 1.2E−02; GRB10, |
| | −4.8E+00, 1.5E−02; PBX1, −4.8E+00, 2.7E−03; RPLP1, −4.7E+00, 8.8E−03; ATP6, −4.7E+00, 4.8E−05; BCL11A, −4.7E+00, 2.6E−02; VASH2, −4.7E+00, 2.6E−02; |
| | BEND3, −4.7E+00, 4.8E−03; HSP90AA1, −4.7E+00, 9.6E−04; RPL3, −4.6E+00, 2.4E−02; ND2, −4.6E+00, 4.3E−03; CNMD, −4.6E+00, 3.8E−02; RPS24, |
| | −4.6E+00, 4.2E−02; RPS18, −4.6E+00, 3.9E−02; TRPS1, −4.5E+00, 1.3E−02; NASP, −4.5E+00, 2.6E−02; SIRT1, −4.5E+00, 1.4E−02; CDKL5, −4.4E+00, 2.7E−02; |
| | TRPC4, −4.4E+00, 1.8E−02; C1GALT1, −4.3E+00, 4.7E−02; MAD2L2, −4.3E+00, 2.7E−03; NCL, −4.2E+00, 1.5E−02; SERPINB9, −4.1E+00, 1.1E−02; SLC1A3, |
| | −3.8E+00, 5.4E−05; CDCA7L, −3.8E+00, 3.9E−02; SLC16A12, −3.8E+00, 8.8E−07; GRTP1, −3.7E+00, 2.4E−02; GLB1L3, −3.7E+00, 3.4E−04; C5orf46, −3.6E+00, |
| | 7.4E−04; COX1, −3.6E+00, 9.2E−06; TMEM64, −3.5E+00, 4.7E−02; LRAT, −3.4E+00, 5.3E−05; VAV3, −3.4E+00, 2.8E−02; S100A10, −3.3E+00, 8.4E−03; TRDN, |
| | −3.2E+00, 3.3E−02; CAPN1, −3.0E+00, 2.4E−02; LOC101926942, −3.0E+00, 7.3E−03; LRMDA, −3.0E+00, 1.5E−02; LINC01098, −3.0E+00, 2.7E−07; SLC25A4, |
| | −2.8E+00, 4.8E−03; NIBAN1, −2.7E+00, 1.1E−02; LOC105369165, −2.6E+00, 4.7E−05; CYP26A1, −2.5E+00, 3.4E−03; HLA-DPB2, −2.4E+00, 2.6E−02; |
| | LOC107985887, −2.4E+00, 7.4E−03; CA8, −2.1E+00, 7.5E−03; LOC105375014, −2.1E+00, 1.9E−02; TMEM220-AS1, −2.1E+00, 1.1E−02; SPINT1, −2.1E+00, |
| | 3.7E−02; TPRA1, −2.1E+00, 1.7E−05; PRLR, −2.0E+00, 2.2E−02; NPY1R, −2.0E+00, 1.3E−02; RIPPLY3, −1.9E+00, 1.5E−04; BHLHE40, −1.8E+00, 4.4E−02; |
| | DPPA3, −1.5E+00, 1.7E−07; ERVH48−1, −1.4E+00, 3.4E−02; LOC107985647, −1.3E+00, 1.9E−02; OSMR, −1.1E+00, 4.9E−02; LOC100505817, −1.1E+00, |
| | 3.6E−02; LOC107985167, −1.1E+00, 4.3E−02; XKR7, −8.4E−01, 4.7E−02; LAT, −6.7E−01, 4.3E−04; LOC105370956, −3.8E−01, 3.7E−02; LINC00898, |
| | −9.9E−02, 2.6E−02; LOC107985849, 1.7E−01, 3.2E−05; LOC105378776, 2.1E−01, 1.0E−02; ZXDA, 2.4E−01, 4.5E−02; LINC01451, 2.9E−01, 2.7E−07; |
| | LOC105379289, 3.1E−01, 2.5E−03; LOC105377978, 3.2E−01, 4.6E−02; LOC105375410, 3.2E−01, 1.5E−02; PANCR, 3.3E−01, 1.6E−02; LOC105374474, |
| | 3.3E−01, 3.6E−03; LOC101929019, 3.7E−01, 7.0E−03; GPR85, 3.9E−01, 5.8E−03; XCL2, 4.1E−01, 1.6E−02; AKR7L, 4.2E−01, 2.4E−02; LOC107986445, 4.5E−01, |
| | 3.7E−02; TBX3, 4.7E−01, 1.7E−02; RSPO3, 5.9E−01, 4.6E−02; RBM24, 6.2E−01, 4.4E−02; GUCA1B, 7.3E−01, 1.9E−02; PITX2, 7.3E−01, 4.5E−02; SKAP1, |
| | 7.5E−01, 2.2E−02; LOC107987271, 7.7E−01, 3.6E−02; LINC00609, 7.7E−01, 1.6E−02; FAS, 8.9E−01, 1.2E−02; ANKRD45, 9.3E−01, 2.1E−02; ATOH8, 9.8E−01, |
| | 2.7E−04; DKK1, 1.0E+00, 1.5E−04; STON1−GTF2A1L, 1.0E+00, 3.4E−02; LOC107984427, 1.0E+00, 3.4E−02; TNNT2, 1.0E+00, 4.0E−02; THNSL2, |
| | 1.1E+00, 3.2E−02; PAX6, 1.1E+00, 2.8E−02; COL28A1, 1.1E+00, 5.3E−03; OSCAR, 1.1E+00, 7.9E−03; POU3F2, 1.1E+00, 4.2E−04; EPHA2, 1.2E+00, |
| | 1.0E−02; GATA2, 1.2E+00, 8.2E−04; GRHL1, 1.2E+00, 3.9E−02; MDGA1, 1.2E+00, 1.3E−03; LOC105374057, 1.2E+00, 9.3E−03; BCHE, 1.3E+00, 1.4E−02; |
| | C4orf22, 1.3E+00, 2.7E−03; APLNR, 1.3E+00, 4.6E−04; TTC6, 1.4E+00, 4.0E−03; LRRC9, 1.4E+00, 4.1E−02; NGFR, 1.4E+00, 4.1E−03; DIO2, 1.4E+00, |
| | 1.8E−05; LOC102724227, 1.5E+00, 3.0E−02; LOC105370302, 1.5E+00, 5.0E−06; LRRN3, 1.5E+00, 3.3E−02; DNAH6, 1.5E+00, 2.3E−02; LOC107984112, |
| | 1.5E+00, 4.1E−02; BMP5, 1.5E+00, 4.5E−03; DLX6, 1.6E+00, 3.8E−06; BICC1, 1.6E+00, 3.0E−02; TNFRSF11B, 1.6E+00, 2.7E−02; DLX6-AS1, 1.6E+00, |
| | 1.3E−06; GATA3, 1.6E+00, 9.2E−04; LOC100130502, 1.6E+00, 5.1E−04; LOC102723436, 1.6E+00, 2.2E−03; LEF1, 1.6E+00, 1.7E−02; CCDC146, 1.6E+00, 2.0E−02; |
| | SCD5, 1.6E+00, 9.4E−03; ZNF664−FAM101A, 1.7E+00, 3.0E−02; LOC107984063, 1.7E+00, 4.5E−02; LINC00869, 1.7E+00, 1.0E−02; TLL1, 1.8E+00, |
| | 8.7E−04; LOC100506885, 1.8E+00, 9.6E−03; MFAP4, 1.8E+00, 8.1E−05; HRK, 1.8E+00, 2.4E−02; LOC105374056, 1.8E+00, 5.1E−04; RGS16, 1.8E+00, |
| | 5.3E−03; CCDC92, 1.9E+00, 2.2E−05; RASGEF1B, 1.9E+00, 3.0E−02; PLPP4, 1.9E+00, 2.0E−04; SYTL5, 1.9E+00, 1.3E−02; GLIS1, 1.9E+00, 8.5E−03; NEK10, |
| | 1.9E+00, 2.1E−02; MANEA, 1.9E+00, 4.1E−02; TCHP, 1.9E+00, 5.5E−03; DLX5, 2.0E+00, 1.6E−05; YPEL4, 2.0E+00, 1.5E−02; BGN, 2.0E+00, 2.2E−05; |
| | GPRIN3, 2.0E+00, 2.4E−02; LOC105378305, 2.0E+00, 1.6E−05; ASXL3, 2.0E+00, 6.9E−04; CPXM1, 2.0E+00, 1.3E−02; NOVA1-AS1, 2.1E+00, 1.3E−02; |
| | HPSE2, 2.1E+00, 4.0E−02; PHC2, 2.1E+00, 9.1E−03; LRRC3B, 2.1E+00, 6.6E−03; DLL3, 2.1E+00, 3.6E−06; FGF14, 2.1E+00, 1.3E−02; LOC107986641, |
| | 2.1E+00, 3.6E−02; LHX5-AS1, 2.1E+00, 3.0E−03; D21S2088E, 2.1E+00, 6.0E−06; LOC101929468, 2.1E+00, 1.8E−02; HIST1H2BM, 2.1E+00, 5.0E−02; |
| | CXXC4, 2.2E+00, 2.7E−03; SMAD7, 2.2E+00, 4.0E−02; SUSD6, 2.2E+00, 1.8E−02; LYRM1, 2.2E+00, 2.5E−03; LRRC34, 2.2E+00, 4.9E−04; MYBL1, 2.2E+00, |
| | 9.2E−03; LINC00290, 2.2E+00, 4.1E−02; PXDNL, 2.3E+00, 9.7E−03; HIST1H4B, 2.3E+00, 6.4E−03; COL5A2, 2.3E+00, 4.9E−02; CFAP44, 2.3E+00, 1.1E−03; |
| | NCKAP5, 2.4E+00, 4.0E−02; IGSF11, 2.4E+00, 2.0E−02; KAT2B, 2.4E+00, 1.4E−03; MMP2, 2.4E+00, 1.2E−02; DGKB, 2.4E+00, 1.9E−03; CCDC102B, |
| | 2.4E+00, 7.4E−03; MAF, 2.4E+00, 1.7E−02; GKAP1, 2.4E+00, 1.9E−02; RNF152, 2.4E+00, 4.0E−02; PLEKHG1, 2.4E+00, 5.7E−03; TFF3, 2.4E+00, 1.8E−02; |
| | HABP4, 2.4E+00, 4.1E−02; PKIG, 2.4E+00, 3.3E−02; NEDD4, 2.5E+00, 4.7E−03; HIST1H2BJ, 2.5E+00, 8.7E−03; SLC6A15, 2.5E+00, 3.2E−04; LRRC37A3, |
| | 2.5E+00, 1.6E−02; ZNF782, 2.5E+00, 2.9E−02; TPBG, 2.5E+00, 6.0E−03; GGH, 2.5E+00, 4.9E−02; STX17, 2.5E+00, 4.9E−02; TSPAN12, 2.5E+00, 1.7E−02; |
| | RBM20, 2.5E+00, 1.9E−04; HIST1H2BO, 2.5E+00, 5.0E−02; JKAMP, 2.5E+00, 2.4E−02; FZD2, 2.5E+00, 1.3E−03; HHAT, 2.6E+00, 5.7E−04; ENC1, 2.6E+00, |
| | 2.3E−03; RGMB, 2.6E+00, 1.9E−02; PCAT14, 2.6E+00, 4.0E−02; TFPI, 2.6E+00, 1.2E−04; PIK3R1, 2.6E+00, 2.1E−02; SRGAP2C, 2.6E+00, 6.8E−04; GATS, |
| | 2.6E+00, 1.9E−02; SULF2, 2.6E+00, 2.6E−02; TUBB6, 2.6E+00, 3.6E−02; SEZ6L, 2.6E+00, 5.2E−07; COL2A1, 2.6E+00, 6.9E−03; POC1B, 2.7E+00, 3.2E−02; |
| | NKD1, 2.7E+00, 2.9E−04; NUCB2, 2.7E+00, 4.4E−02; HIST1H4F, 2.7E+00, 4.0E−02; OTX2-AS1, 2.7E+00, 2.2E−04; TMEM232, 2.7E+00, 2.6E−02; FAM19A1, |
| | 2.7E+00, 1.7E−02; BLCAP, 2.7E+00, 3.1E−04; C3orf70, 2.7E+00, 6.3E−04; SOX6, 2.8E+00, 1.4E−04; EBF2, 2.8E+00, 1.6E−07; COLEC12, 2.8E+00, 5.6E−04; |
| | KLHL2, 2.8E+00, 4.4E−02; FBN1, 2.8E+00, 7.1E−03; ADAMTS17, 2.8E+00, 1.1E−02; SUFU, 2.8E+00, 2.5E−02; GRK5, 2.8E+00, 9.7E−04; RAP2B, 2.9E+00, |
| | 5.9E−03; LOC107984581, 2.9E+00, 4.9E−02; KCNIP1, 2.9E+00, 6.4E−03; FMN2, 2.9E+00, 1.1E−02; PCOLCE2, 2.9E+00, 3.5E−02; CPE, 2.9E+00, 3.1E−03; |
| | WDYHV1, 2.9E+00, 1.6E−02; LOC107983974, 2.9E+00, 5.6E−06; TTLL7, 2.9E+00, 4.6E−02; AHDC1, 2.9E+00, 4.8E−02; TNFRSF19, 3.0E+00, 5.7E−05; |
| | LOC107985879, 3.0E+00, 6.5E−07; RTN1, 3.0E+00, 1.6E−02; GXYLT1, 3.0E+00, 3.7E−02; ZNF518B, 3.0E+00, 9.2E−03; SLC39A8, 3.0E+00, 3.4E−06; |
| | SMAD1, 3.0E+00, 3.2E−02; COL13A1, 3.0E+00, 9.8E−04; PAQR3, 3.0E+00, 5.9E−03; NCALD, 3.0E+00, 2.4E−03; METTL2B, 3.0E+00, 4.0E−02; HECTD2, |
| | 3.1E+00, 3.0E−04; RAI1, 3.1E+00, 1.2E−04; PLXNA2, 3.1E+00, 8.1E−06; MDFIC, 3.1E+00, 5.4E−05; LOC105374693, 3.1E+00, 2.0E−03; CCNG2, 3.1E+00, |
| | 1.5E−05; IGDCC4, 3.1E+00, 1.5E−02; PPME1, 3.1E+00, 4.9E−02; LOC107985783, 3.1E+00, 5.9E−03; TNC, 3.1E+00, 1.9E−04; H3C8, 3.1E+00, 4.0E−02; |
| | B3GLCT, 3.1E+00, 8.2E−03; WIPF1, 3.1E+00, 5.9E−04; LRRN2, 3.1E+00, 9.2E−04; TANC1, 3.1E+00, 1.9E−02; PROS1, 3.2E+00, 5.7E−07; FBLN7, 3.2E+00, |
| | 5.6E−09; DOK6, 3.2E+00, 7.7E−03; ARL13B, 3.2E+00, 4.9E−02; SYT10, 3.2E+00, 1.2E−02; ANKRD6, 3.2E+00, 3.6E−02; PCBP1-AS1, 3.2E+00, 4.7E−02; |
| | FRMD6, 3.2E+00, 3.4E−03; MRC2, 3.2E+00, 1.3E−02; SNRPN, 3.2E+00, 3.5E−03; FHDC1, 3.3E+00, 1.4E−02; RNF165, 3.3E+00, 2.7E−03; PREX1, 3.3E+00, |
| | 2.7E−04; H2AC21, 3.3E+00, 2.5E−03; ATP9A, 3.3E+00, 9.1E−03; TANC2, 3.3E+00, 4.9E−02; NXPH2, 3.3E+00, 3.1E−02; KHDRBS3, 3.3E+00, 2.8E−02; |
| | ANTXR2, 3.4E+00, 3.9E−06; ZBTB18, 3.4E+00, 8.6E−06; C1orf112, 3.4E+00, 4.1E−02; MAP3K20, 3.4E+00, 5.3E−04; HIRA, 3.4E+00, 4.8E−02; TCF21, |
| | 3.4E+00, 4.5E−10; TNFRSF10B, 3.4E+00, 1.1E−02; TSHZ3, 3.4E+00, 3.7E−04; LOC101927947, 3.4E+00, 9.3E−06; SMOC1, 3.4E+00, 2.1E−04; FAM184A, |
| | 3.4E+00, 4.4E−02; EFCAB13, 3.5E+00, 1.7E−02; BAHCC1, 3.5E+00, 5.5E−10; FAM110B, 3.5E+00, 2.4E−03; FAM149B1, 3.5E+00, 7.4E−03; H3C7, 3.5E+00, |
| | 4.4E−02; FBXW8, 3.5E+00, 2.8E−02; IPO5P1, 3.5E+00, 1.0E−02; RGL1, 3.5E+00, 3.1E−03; SATB1, 3.5E+00, 5.5E−03; CEP112, 3.5E+00, 2.3E−02; GEN1, |
| | 3.5E+00, 1.2E−02; MEIS2, 3.6E+00, 3.5E−04; SEC24B, 3.6E+00, 3.5E−02; SHROOM2, 3.6E+00, 1.0E−02; AFAP1, 3.6E+00, 6.9E−03; DLK1, 3.6E+00, |
| | 1.3E−08; MALAT1, 3.6E+00, 8.6E−06; H2BC10, 3.6E+00, 5.5E−04; ANAPC10, 3.6E+00, 4.1E−02; FRMD3, 3.6E+00, 1.7E−04; NFASC, 3.6E+00, 1.0E−02; LRIG3, |
| | 3.6E+00, 2.4E−04; SIL1, 3.6E+00, 5.6E−04; NETO2, 3.7E+00, 2.3E−03; HIST1H2BK, 3.7E+00, 9.5E−03; GAREM1, 3.7E+00, 4.0E−02; FBXL4, 3.7E+00, |
| | 1.6E−02; ARHGAP10, 3.7E+00, 9.3E−03; ST8SIA2, 3.7E+00, 1.4E−05; COL11A1, 3.7E+00, 3.8E−02; FBLN1, 3.7E+00, 9.5E−03; LOC107983984, 3.8E+00, 4.6E−02; |
| | PTPRM, 3.8E+00, 3.3E−02; KIF26B, 3.8E+00, 4.4E−05; TBC1D14, 3.8E+00, 4.5E−03; TRAM2, 3.8E+00, 7.9E−04; COL1A2, 3.8E+00, 3.6E−02; UTP18, |
| | 3.8E+00, 2.9E−02; NTS, 3.8E+00, 4.2E−03; CBFB, 3.8E+00, 9.2E−03; PARK2, 3.8E+00, 2.4E−02; GREM1, 3.9E+00, 7.9E−09; NRCAM, 3.9E+00, 3.1E−02; |
| | YAF2, 3.9E+00, 2.5E−06; AMOTL1, 3.9E+00, 7.8E−03; CSRNP3, 3.9E+00, 4.1E−02; TAF3, 3.9E+00, 4.4E−02; WLS, 3.9E+00, 2.8E−06; HEG1, 3.9E+00, |
| | 1.3E−04; HMG20A, 3.9E+00, 9.2E−03; FZD5, 3.9E+00, 3.8E−02; FRZB, 3.9E+00, 3.9E−06; CYP20A1, 3.9E+00, 1.6E−02; DNAJC3, 4.0E+00, 1.0E−02; PTCH1, |
| | 4.0E+00, 9.5E−03; PAPPA, 4.0E+00, 1.7E−03; VGLL4, 4.0E+00, 2.0E−02; E2F7, 4.0E+00, 1.0E−02; H3C11, 4.0E+00, 3.1E−02; FST, 4.0E+00, 5.4E−04; REV3L, |
| | 4.0E+00, 3.0E−02; TP53I11, 4.0E+00, 5.9E−03; FAM171A1, 4.0E+00, 2.2E−02; CCDC50, 4.0E+00, 1.7E−03; PHLDB2, 4.0E+00, 1.2E−03; ZC3H12C, 4.0E+00, |
| | 7.7E−03; HIST1H2AK, 4.0E+00, 8.1E−05; DGKH, 4.0E+00, 3.1E−02; LRRC8B, 4.1E+00, 8.4E−03; COL4A1, 4.1E+00, 1.0E−02; COL4A2, 4.1E+00, 1.8E−02; |
| | MECOM, 4.1E+00, 5.5E−05; PROM1, 4.1E+00, 4.0E−02; MN1, 4.1E+00, 4.9E−06; ASAP2, 4.1E+00, 2.7E−02; ECHDC1, 4.2E+00, 1.5E−03; WASF3, 4.2E+00, |
| | 5.1E−04; H3C12, 4.2E+00, 1.4E−04; PHTF1, 4.2E+00, 5.7E−03; EMSY, 4.2E+00, 4.2E−02; TTI1, 4.2E+00, 1.0E−02; RGS12, 4.2E+00, 3.2E−03; DEPDC1B, |
| | 4.2E+00, 4.7E−02; H4C12, 4.2E+00, 1.8E−03; ZDHHC21, 4.2E+00, 2.7E−02; TRMT9B, 4.2E+00, 2.0E−10; MBOAT2, 4.2E+00, 3.4E−02; SLF1, 4.2E+00, 4.0E−03; |
| | ADAMTS6, 4.3E+00, 7.9E−04; MCF2L, 4.3E+00, 3.6E−03; CDK17, 4.3E+00, 1.6E−02; LOC105374322, 4.3E+00, 1.5E−03; SHB, 4.3E+00, 2.9E−03; |
| | SCAPER, 4.3E+00, 1.4E−02; CXXC5, 4.3E+00, 2.4E−04; C1QTNF3-AMACR, 4.3E+00, 8.2E−03; ADD1, 4.3E+00, 1.6E−02; DENND2B, 4.4E+00, 7.0E−05; AFF3, |
| | 4.4E+00, 6.1E−09; COCH, 4.4E+00, 1.3E−06; GJC1, 4.4E+00, 1.8E−02; TRIP11, 4.4E+00, 1.4E−02; MAP2K5, 4.4E+00, 1.2E−02; C15orf41, 4.4E+00, |
| | 1.7E−02; EP400, 4.4E+00, 3.8E−02; TTC37, 4.4E+00, 3.1E−02; HDAC4, 4.5E+00, 4.7E−02; NAALAD2, 4.5E+00, 3.4E−05; H2AC13, 4.5E+00, 2.3E−03; |
| | LOC107986007, 4.5E+00, 5.3E−04; LOC285696, 4.5E+00, 1.1E−03; TSPAN18, 4.5E+00, 3.1E−04; C11orf49, 4.5E+00, 3.1E−02; SPRED1, 4.5E+00, 2.2E−02; |
| | SOX2-OT, 4.5E+00, 1.0E−02; ZNF516, 4.5E+00, 8.2E−04; FBXL17, 4.5E+00, 4.6E−02; IVNS1ABP, 4.5E+00, 3.6E−02; CDON, 4.5E+00, 7.9E−03; TRIM44, |
| | 4.5E+00, 1.2E−02; DPH6, 4.5E+00, 4.1E−02; LOC105370982, 4.5E+00, 2.5E−09; ZNF407, 4.6E+00, 1.6E−02; LCORL, 4.6E+00, 4.6E−02; ZBTB20, 4.6E+00, |
| | 2.4E−02; SHISA2, 4.6E+00, 1.5E−06; POU6F2, 4.6E+00, 2.3E−04; LSM14A, 4.6E+00, 4.9E−02; EFNB2, 4.6E+00, 2.9E−04; DLEU2, 4.6E+00, 1.4E−03; DUSP6, |
| | 4.6E+00, 2.5E−10; RSRC1, 4.6E+00, 3.3E−02; SAMD4A, 4.7E+00, 5.6E−05; ARL3, 4.7E+00, 6.3E−04; ELP4, 4.7E+00, 4.9E−02; CLMP, 4.7E+00, 2.0E−03; |
| | WDR70, 4.7E+00, 2.2E−02; CREBBP, 4.7E+00, 3.4E−02; CPD, 4.7E+00, 2.2E−03; USP3, 4.7E+00, 1.7E−05; NSD2, 4.7E+00, 4.4E−02; GTDC1, 4.7E+00, 2.1E−02; |
| | NFAT5, 4.7E+00, 1.9E−02; NCAM1, 4.8E+00, 6.5E−05; SLC44A5, 4.8E+00, 2.0E−03; DANT2, 4.8E+00, 4.6E−02; H2AC16, 4.8E+00, 2.3E−03; PLEKHA5, |
| | 4.8E+00, 2.2E−02; CCBE1, 4.8E+00, 3.0E−06; CEP350, 4.8E+00, 2.5E−02; PDE3B, 4.8E+00, 4.8E−03; HIP1, 4.8E+00, 4.8E−02; UGGT2, 4.8E+00, 4.3E−02; |
| | SMAD6, 4.8E+00, 2.5E−07; EML1, 4.8E+00, 1.0E−03; DENND2A, 4.8E+00, 1.0E−05; MIPOL1, 4.8E+00, 2.6E−02; TULP4, 4.8E+00, 4.0E−02; EYA1, 4.8E+00, |
| | 8.9E−05; SESTD1, 4.9E+00, 5.9E−04; HACD3, 4.9E+00, 3.3E−02; PDE10A, 4.9E+00, 3.1E−04; H2BC7, 4.9E+00, 2.6E−04; ZC4H2, 4.9E+00, 1.8E−02; |
| | COL26A1, 4.9E+00, 1.1E−02; CDK19, 4.9E+00, 1.1E−02; SRRM4, 4.9E+00, 8.7E−06; SLC7A11, 4.9E+00, 3.5E−03; RASA1, 4.9E+00, 7.8E−04; TTC17, |
| | 4.9E+00, 4.2E−02; HS2ST1, 4.9E+00, 4.1E−02; RASSF8, 4.9E+00, 1.2E−03; AP3B1, 5.0E+00, 3.0E−02; INVS, 5.0E+00, 6.5E−03; HDAC8, 5.0E+00, 1.6E−02; |
| | SPIDR, 5.0E+00, 3.7E−02; BMPER, 5.0E+00, 1.3E−11; APP, 5.0E+00, 3.0E−02; LOC105377862, 5.0E+00, 6.5E−03; MYLK, 5.0E+00, 6.9E−04; CALD1, |
| | 5.0E+00, 3.2E−02; GNAS, 5.0E+00, 3.4E−02; DYM, 5.1E+00, 6.8E−03; RANBP1, 5.1E+00, 5.5E−03; PDS5B, 5.1E+00, 1.9E−02; FANCI, 5.1E+00, 1.1E−02; |
| | HIST1H2AC, 5.1E+00, 4.5E−03; PTK2, 5.1E+00, 2.0E−02; LRP2, 5.2E+00, 2.5E−10; BCAS3, 5.2E+00, 2.0E−02; TFAP2A, 5.2E+00, 9.6E−14; NT5C2, 5.2E+00, |
| | 1.5E−03; SNTB1, 5.2E+00, 2.8E−03; MAP2K6, 5.2E+00, 3.8E−03; NEDD9, 5.2E+00, 2.0E−10; MACROH2A1, 5.2E+00, 1.4E−05; R3HDM1, 5.2E+00, 2.7E−02; |
| | ASCC3, 5.2E+00, 3.4E−02; AN06, 5.2E+00, 3.3E−03; STK3, 5.2E+00, 1.0E−02; BTBD9, 5.2E+00, 6.5E−03; ESRRG, 5.3E+00, 4.9E−08; RFX3, 5.3E+00, 2.7E−02; |
| | MAST4, 5.3E+00, 1.2E−04; IMMP2L, 5.3E+00, 2.6E−02; LINC01021, 5.3E+00, 4.8E−03; ASTN1, 5.3E+00, 1.7E−05; P3H2, 5.3E+00, 4.7E−04; ZNF385B, |
| | 5.3E+00, 5.4E−04; UBE2E3, 5.3E+00, 6.1E−03; NF1, 5.3E+00, 1.6E−02; H3C4, 5.3E+00, 3.1E−03; HIBCH, 5.3E+00, 2.6E−03; RAD51B, 5.4E+00, 2.7E−02; |
| | LINC01515, 5.4E+00, 3.5E−03; LOC107986022, 5.4E+00, 1.9E−06; DLEU1, 5.4E+00, 1.9E−03; ASPH, 5.4E+00, 1.2E−02; FLNC, 5.4E+00, 3.8E−11; SSBP3, |
| | 5.4E+00, 1.7E−02; IGDCC3, 5.4E+00, 2.0E−07; DMD, 5.4E+00, 8.1E−04; RBFOX2, 5.4E+00, 1.4E−02; CCND2, 5.4E+00, 8.6E−03; HMCN1, 5.5E+00, 7.8E−08; |
| | H4C8, 5.5E+00, 3.1E−04; RAB28, 5.5E+00, 3.0E−03; STX8, 5.5E+00, 4.5E−04; GREB1, 5.5E+00, 7.1E−06; PCDH11X, 5.5E+00, 3.4E−02; SYNE2, 5.5E+00, |
| | 1.2E−02; CHD1, 5.5E+00, 7.6E−03; SMAD5, 5.5E+00, 5.4E−04; EPB41L2, 5.5E+00, 6.4E−03; HIST1H2AH, 5.5E+00, 1.3E−03; PLCH1, 5.6E+00, 1.3E−02; |
| | POLA1, 5.6E+00, 1.3E−02; H3C10, 5.6E+00, 5.4E−05; TCF12, 5.6E+00, 7.4E−03; NOVA1, 5.6E+00, 1.4E−03; RNGTT, 5.6E+00, 4.8E−03; GSK3B, 5.6E+00, |
| | 7.6E−03; ZFAND3, 5.6E+00, 1.2E−02; TMEM123, 5.7E+00, 9.4E−05; MMS22L, 5.7E+00, 5.9E−03; CEP78, 5.7E+00, 3.2E−04; ZMIZ1, 5.7E+00, 2.0E−03; NES, |
| | 5.7E+00, 4.7E−03; CTNND2, 5.7E+00, 1.3E−02; UBE21, 5.7E+00, 2.0E−03; GNAQ, 5.7E+00, 9.4E−03; SALL1, 5.7E+00, 2.1E−06; CRADD, 5.7E+00, 1.6E−03; |
| | RBMS1, 5.7E+00, 8.8E−03; GRIK3, 5.8E+00, 7.8E−11; ZNF148, 5.8E+00, 9.2E−04; PTPRN2, 5.8E+00, 1.2E−02; ZNF664, 5.8E+00, 1.6E−06; ID1, 5.8E+00, |
| | 7.5E−05; CDK6, 5.8E+00, 2.0E−03; SDC2, 5.8E+00, 5.1E−04; H2AC8, 5.8E+00, 8.5E−04; MED12L, 5.9E+00, 7.3E−04; LIMCH1, 5.9E+00, 1.8E−04; DZIP1, |
| | 5.9E+00, 1.5E−05; TSPAN5, 5.9E+00, 2.5E−04; CRACD, 5.9E+00, 4.5E−04; PTN, 5.9E+00, 2.6E−05; DIAPH3, 6.0E+00, 7.2E−03; SLC5A3, 6.0E+00, 7.0E−06; |
| | SOX11, 6.0E+00, 5.9E−04; FAM13A, 6.1E+00, 9.8E−04; PREX2, 6.1E+00, 5.4E−04; DPY19L3, 6.1E+00, 3.0E−04; SEMASA, 6.1E+00, 1.5E−04; AFF2, |
| | 6.1E+00, 5.0E−05; H4C5, 6.1E+00, 7.7E−03; EXOC4, 6.2E+00, 2.9E−03; NCOA1, 6.2E+00, 3.4E−04; MIR924HG, 6.2E+00, 1.4E−02; PPFIBP1, 6.2E+00, 2.8E−05; |
| | STIM2, 6.2E+00, 1.7E−07; CAMKMT, 6.2E+00, 4.3E−03; DLG1, 6.3E+00, 2.0E−03; MACROD2, 6.3E+00, 1.2E−03; NRG1, 6.3E+00, 1.2E−07; KALRN, |
| | 6.3E+00, 6.9E−05; ZEB1, 6.3E+00, 7.9E−14; EPHA4, 6.3E+00, 7.7E−04; CASK, 6.3E+00, 1.1E−03; LOC100420587, 6.3E+00, 3.1E−03; NRIP1, 6.3E+00, 3.6E−06; |
| | PAMR1, 6.4E+00, 6.5E−09; FZD3, 6.4E+00, 9.1E−05; UBE3D, 6.4E+00, 1.2E−03; ADAM23, 6.5E+00, 1.3E−04; TMEM132C, 6.5E+00, 2.5E−15; GRIP1, |
| | 6.6E+00, 6.3E−04; LIX1, 6.7E+00, 2.1E−25; RNF217, 6.7E+00, 7.7E−09; VPS13B, 6.7E+00, 6.8E−04; TIAM1, 6.7E+00, 3.7E−04; H1-5, 6.7E+00, 9.5E−03; |
| | NOTCH2, 6.7E+00, 6.6E−08; SLC25A13, 6.8E+00, 5.0E−05; H2AFY2, 6.8E+00, 1.2E−11; GRAMD1B, 6.8E+00, 3.0E−05; H1-2, 6.8E+00, 3.1E−03; ROR2, |
| | 6.8E+00, 3.7E−08; DST, 6.8E+00, 4.4E−05; H2AC20, 6.8E+00, 7.9E−04; STON2, 6.9E+00, 5.6E−07; RFC3, 6.9E+00, 4.1E−04; AKAP13, 6.9E+00, 4.2E−04; |
| | TLN2, 6.9E+00, 7.1E−05; KAZN, 7.0E+00, 7.0E−04; DLG5, 7.0E+00, 2.1E−06; HIST1H1E, 7.0E+00, 1.0E−03; SEPTIN9, 7.0E+00, 1.8E−04; ADGRL2, 7.0E+00, |
| | 2.3E−04; SVIL, 7.0E+00, 8.7E−07; CUX1, 7.0E+00, 1.2E−04; ANXA5, 7.0E+00, 2.7E−05; ID4, 7.1E+00, 2.5E−10; ME3, 7.1E+00, 1.4E−14; NLGN1, 7.1E+00, |
| | 7.8E−04; FIGN, 7.1E+00, 2.0E−04; TBL1X, 7.2E+00, 1.2E−04; CADPS, 7.2E+00, 2.0E−05; SLC4A7, 7.2E+00, 5.1E−06; ELMO1, 7.2E+00, 1.0E−07; PRKG1, |
| | 7.3E+00, 7.9E−04; GSE1, 7.3E+00, 1.5E−04; MED13L, 7.3E+00, 1.7E−04; LSAMP, 7.4E+00, 1.2E−04; YAP1, 7.4E+00, 6.0E−05; HIST1H2BC, 7.4E+00, |
| | 1.1E−06; FBXL7, 7.4E+00, 5.8E−05; FOXP2, 7.5E+00, 1.3E−20; BCKDHB, 7.5E+00, 8.8E−05; SMYD3, 7.6E+00, 5.2E−05; MID1, 7.7E+00, 1.8E−05; H2AC17, |
| | 7.8E+00, 1.7E−04; NEO1, 7.8E+00, 4.6E−06; LAMB1, 7.8E+00, 6.5E−07; TCF4, 7.9E+00, 1.0E−05; STOX2, 7.9E+00, 1.7E−07; UNC5C, 7.9E+00, 3.4E−10; |
| | SH3RF1, 7.9E+00, 9.1E−08; SIPA1L2, 7.9E+00, 7.4E−11; PSD3, 7.9E+00, 1.6E−05; AKT3, 8.0E+00, 1.2E−05; PEAK1, 8.0E+00, 3.0E−05; FRMD4A, 8.0E+00, |
| | 4.7E−06; IL17RD, 8.0E+00, 1.5E−12; CHD7, 8.0E+00, 1.4E−05; IL1RAPL1, 8.0E+00, 4.7E−07; MMP16, 8.1E+00, 5.1E−06; CALB1, 8.2E+00, 1.1E−07; GRIK2, |
| | 8.4E+00, 1.4E−05; LPP, 8.4E+00, 5.1E−06; PPP3CA, 8.5E+00, 1.3E−06; ARHGAP28, 8.5E+00, 2.1E−13; ST6GALNAC3, 8.5E+00, 8.5E−06; ZFHX4, 8.5E+00, |
| | 3.1E−11; MIAT, 8.6E+00, 6.6E−07; H2AC11, 8.6E+00, 4.6E−07; KIRREL1, 8.7E+00, 2.9E−09; PRSS23, 8.7E+00, 2.8E−17; CTNNA2, 8.7E+00, 5.5E−06; |
| | SMAD2, 8.8E+00, 6.4E−07; FAM155A, 8.8E+00, 1.5E−07; SIX3, 8.8E+00, 1.7E−29; H3C2, 8.8E+00, 3.8E−05; PTCHD4, 8.9E+00, 4.9E−15; PALLD, 9.0E+00, |
| | 3.2E−09; BCAT1, 9.1E+00, 3.7E−07; CNTNAP2, 9.1E+00, 5.6E−07; SEPTIN11, 9.2E+00, 3.5E−08; TLE4, 9.3E+00, 3.6E−08; ID2, 9.4E+00, 7.5E−14; ATP2B1, |
| | 9.7E+00, 2.3E−09; RASGRF2, 9.7E+00, 1.2E−15; NR6A1, 9.7E+00, 2.0E−09; SETBP1, 9.8E+00, 1.4E−09; ZNF521, 9.8E+00, 2.3E−08; FHOD3, 9.9E+00, 5.5E−11; |
| | MAML3, 9.9E+00, 2.5E−09; TTC28, 9.9E+00, 2.3E−08; HS6ST2, 9.9E+00, 1.7E−11; AUTS2, 1.0E+01, 1.2E−12; LOC105377979, 1.0E+01, 2.3E−43; |
| | PTPRD, 1.0E+01, 1.8E−08; SLIT2, 1.0E+01, 2.8E−10; GLIS3, 1.0E+01, 2.2E−19; EPHA7, 1.0E+01, 1.8E−13; WWOX, 1.0E+01, 8.7E−10; CDH2, 1.0E+01, |
| | 1.5E−09; MIR325HG, 1.0E+01, 1.7E−12; ST6GALNAC5, 1.0E+01, 2.7E−11; SC5D, 1.0E+01, 9.4E−12; TMTC2, 1.0E+01, 1.4E−09; PAM, 1.1E+01, 8.9E−11; LRRC4C, |
| | 1.1E+01, 1.7E−08; PRKD1, 1.1E+01, 3.8E−11; CAMK2D, 1.1E+01, 1.9E−14; NEBL, 1.1E+01, 6.5E−11; PKNOX2, 1.1E+01, 5.8E−16; TEAD1, 1.1E+01, 7.3E−10; |
| | SSBP2, 1.1E+01, 3.9E−10; ZC3H12B, 1.1E+01, 1.1E−09; SEMA6A, 1.1E+01, 1.0E−13; ZNF423, 1.1E+01, 1.2E−11; SLC35F1, 1.2E+01, 3.9E−14; SYNDIG1, |
| | 1.2E+01, 5.2E−14; PGAP1, 1.2E+01, 4.2E−13; ZSWIM6, 1.2E+01, 5.2E−14; DACH1, 1.2E+01, 3.7E−20; PDE4D, 1.2E+01, 3.9E−16; CADM1, 1.2E+01, 9.4E−15; |
| | CNTN4, 1.3E+01, 1.2E−15; LMX1A, 1.3E+01, 2.1E−37; MACF1, 1.4E+01, 1.1E−21; GPC3, 1.4E+01, 1.3E−13; GLI3, 1.4E+01, 1.1E−18; CDH11, 1.4E+01, |
| | 1.1E−24; TENM4, 1.4E+01, 1.7E−14; PTPN13, 1.4E+01, 3.5E−17; DCC, 1.4E+01, 5.3E−30; ZEB2, 1.5E+01, 1.1E−30; NNAT, 1.5E+01, 9.7E−26; SDK2, |
| | 1.5E+01, 1.9E−22; ID3, 1.5E+01, 6.3E−23; NAV3, 1.6E+01, 1.1E−24; TENM3, 1.6E+01, 7.5E−28; PCDH11Y, 1.7E+01, 2.8E−36; GPC6, 1.7E+01, 1.0E−22; |
| | CDK14, 1.8E+01, 6.0E−26; SOX5, 1.8E+01, 1.4E−25; CDH6, 1.9E+01, 1.1E−30; NKAIN3, 2.0E+01, 4.0E−30; MAP2, 2.1E+01, 1.3E−36; FBN2, 2.2E+01, 4.6E−39; |
| | MAPK10, 2.4E+01, 7.8E−44; EFNA5, 2.7E+01, 2.0E−69; GREB1L, 3.3E+01, 4.4E−83; PRTG, 3.4E+01, 5.8E−81; |
| 3 | PTMA, −1.5E+01, 4.9E−109; RPS8, −1.3E+01, 5.2E−59; GSTP1, −1.3E+01, 1.7E−47; RPL8, −1.2E+01, 4.1E−42; HSP90AA1, −1.2E+01, 4.0E−65; RPL6, |
| | −1.2E+01, 2.5E−42; HSP90AB1, −1.2E+01, 8.7E−63; HSPA8, −1.2E+01, 2.3E−45; RPL4, −1.2E+01, 1.5E−45; NPM1, −1.2E+01, 7.9E−54; RPS6, −1.1E+01, |
| | 1.6E−50; TPT1, −1.1E+01, 2.1E−38; ACTG1, −1.1E+01, 5.3E−38; GAPDH, −1.1E+01, 1.6E−47; RPL3, −1.1E+01, 5.1E−39; FTH1, −1.1E+01, 2.1E−34; RPL10A, |
| | −1.1E+01, 7.9E−35; RPL34, −1.1E+01, 1.5E−33; RPL13, −1.1E+01, 7.9E−35; RPLP1, −1.1E+01, 7.4E−39; H4C3, −1.1E+01, 3.8E−29; NCL, −1.1E+01, 1.0E−44; |
| | ACTB, −1.1E+01, 4.3E−47; RPL23, −1.1E+01, 5.8E−34; RPL14, −1.1E+01, 7.2E−33; RPL11, −1.1E+01, 7.9E−35; RPS15A, −1.1E+01, 7.6E−33; PFN1, −1.0E+01, |
| | 4.2E−32; RPL35A, −1.0E+01, 1.7E−32; RPS3, −1.0E+01, 3.3E−32; EIF4G2, −1.0E+01, 3.8E−33; RPL13A, −1.0E+01, 5.9E−38; RPS27A, −1.0E+01, 1.8E−31; |
| | RPS12, −1.0E+01, 1.5E−30; RPS23, −1.0E+01, 5.6E−30; RPL12, −1.0E+01, 1.9E−31; RPS3A, −1.0E+01, 1.6E−30; RPL19, −1.0E+01, 9.5E−31; HSP90B1, |
| | −1.0E+01, 5.2E−35; RPS16, −1.0E+01, 4.0E−30; KRT18, −1.0E+01, 6.2E−34; RPL5, −1.0E+01, 1.6E−30; CALM1, −1.0E+01, 2.8E−29; RPS7, −1.0E+01, 4.6E−30; |
| | RACK1, −1.0E+01, 2.3E−29; EEF1A1, −9.9E+00, 4.3E−55; POU5F1, −9.9E+00, 2.8E−29; RPS13, −9.9E+00, 3.0E−31; RPS14, −9.8E+00, 3.4E−28; RPL21, |
| | −9.8E+00, 1.2E−28; TUBB, −9.8E+00, 3.0E−29; SET, −9.8E+00, 5.1E−30; RPS18, −9.6E+00, 1.4E−29; HSPD1, −9.6E+00, 4.2E−30; RPS15, −9.4E+00, 9.6E−27; |
| | CANX, −9.4E+00, 4.0E−28; FTL, −9.4E+00, 5.8E−27; GJA1, −9.4E+00, 5.4E−26; LIN28A, −9.3E+00, 1.3E−31; RPS24, −9.3E+00, 2.7E−26; RPS11, −9.3E+00, |
| | 2.5E−26; HMGA1, −9.2E+00, 2.1E−25; RPSA, −9.1E+00, 4.9E−24; RPL15, −9.1E+00, 1.8E−24; RPS4X, −9.1E+00, 3.2E−25; RPS19, −9.0E+00, 3.1E−24; CD24, |
| | −9.0E+00, 8.8E−30; YWHAZ, −9.0E+00, 5.8E−24; RPS27, −9.0E+00, 1.2E−23; RPL18, −9.0E+00, 1.5E−23; H2AZ1, −8.9E+00, 6.0E−24; RPL31, −8.9E+00, 3.1E−23; |
| | TMSB4X, −8.8E+00, 1.6E−23; RPL7, −8.8E+00, 5.6E−22; HNRNPA1, −8.7E+00, 7.9E−30; RPL24, −8.7E+00, 2.2E−22; KRT8, −8.7E+00, 2.0E−27; EN01, |
| | −8.7E+00, 2.1E−22; PRDX1, −8.6E+00, 2.4E−22; SSB, −8.5E+00, 4.8E−23; RPL7A, −8.5E+00, 1.4E−21; RPL30, −8.5E+00, 1.1E−21; TMSB10, −8.4E+00, |
| | 1.8E−22; STMN1, −8.4E+00, 3.1E−22; RPS17, −8.4E+00, 7.9E−21; RAN, −8.4E+00, 3.8E−22; SERBP1, −8.3E+00, 2.8E−22; H3F3B, −8.3E+00, 1.5E−22; RPL32, |
| | −8.3E+00, 6.4E−20; CALM2, −8.2E+00, 2.4E−20; RPL28, −8.2E+00, 1.0E−19; RPLP2, −8.1E+00, 8.6E−20; HNRNPU, −8.1E+00, 3.9E−23; PABPC1, −8.1E+00, |
| | 3.9E−23; TPM3, −8.0E+00, 2.9E−19; RPL10, −8.0E+00, 2.9E−19; SCD, −8.0E+00, 5.0E−20; HNRNPA2B1, −8.0E+00, 1.8E−32; HSPA5, −7.9E+00, 4.7E−18; |
| | RPLP0, −7.8E+00, 3.4E−18; CFL1, −7.8E+00, 2.6E−20; TDGF1, −7.7E+00, 8.9E−20; RPL35, −7.7E+00, 7.6E−18; RPS9, −7.7E+00, 4.7E−18; RPL26, −7.7E+00, |
| | 4.4E−18; UBB, −7.6E+00, 8.8E−19; LDHB, −7.6E+00, 7.5E−18; YBX1, −7.6E+00, 5.7E−17; NACA, −7.5E+00, 5.3E−17; FAU, −7.5E+00, 1.7E−17; EPCAM, |
| | −7.4E+00, 5.7E−17; HMGB1, −7.4E+00, 3.1E−16; MYL6, −7.4E+00, 2.0E−16; HNRNPK, −7.3E+00, 2.1E−16; PARP1, −7.3E+00, 9.0E−16; CSDE1, −7.3E+00, 7.7E−16; |
| | EEF2, −7.3E+00, 1.8E−15; YWHAE, −7.2E+00, 1.9E−16; RPS2, −7.2E+00, 2.1E−15; L1TD1, −7.2E+00, 6.9E−23; ESRG, −7.2E+00, 4.7E−16; NUCKS1, |
| | −7.2E+00, 5.7E−17; RPS5, −7.2E+00, 2.7E−15; HSPA4, −7.2E+00, 3.1E−15; EEF1G, −7.1E+00, 4.4E−15; RPL27A, −7.1E+00, 2.1E−15; RPL29, −7.1E+00, 4.2E−16; |
| | RPL36, −7.1E+00, 2.6E−15; PDIA3, −7.1E+00, 1.3E−14; HMGB2, −7.0E+00, 1.6E−16; ATP5F1B, −7.0E+00, 1.9E−15; EEF1B2, −7.0E+00, 1.9E−15; H1-5, |
| | −6.9E+00, 8.5E−13; TUBA1B, −6.9E+00, 4.4E−15; PEBP1, −6.9E+00, 1.2E−17; TKT, −6.8E+00, 3.6E−15; NASP, −6.8E+00, 1.9E−15; CCT4, −6.8E+00, 9.7E−15; |
| | TPI1, −6.8E+00, 1.2E−15; RPS20, −6.8E+00, 6.5E−14; H1-3, −6.8E+00, 2.4E−12; CYP51A1, −6.7E+00, 2.1E−15; SRSF3, −6.7E+00, 5.5E−14; ATP5F1A, |
| | −6.7E+00, 8.9E−14; CBX5, −6.7E+00, 5.3E−14; CCT3, −6.7E+00, 8.8E−14; PPIA, −6.6E+00, 2.2E−13; TARS1, −6.6E+00, 1.4E−13; SPP1, −6.6E+00, 1.1E−15; |
| | RPL27, −6.6E+00, 4.4E−15; CALR, −6.6E+00, 4.7E−13; MDK, −6.5E+00, 2.3E−15; RPS28, −6.5E+00, 1.8E−15; MORF4L1, −6.3E+00, 2.3E−12; SEPHS1, |
| | −6.3E+00, 2.1E−12; SF3B1, −6.3E+00, 1.6E−11; CCT5, −6.2E+00, 8.2E−12; TCP1, −6.2E+00, 1.0E−11; RPL18A, −6.2E+00, 1.5E−12; NORAD, −6.1E+00, 8.6E−13; |
| | PSMA4, −6.1E+00, 7.4E−15; S100A10, −6.1E+00, 1.4E−18; PGRMC1, −6.1E+00, 8.1E−13; RPS21, −6.1E+00, 2.6E−13; NDUFS5, −6.1E+00, 9.0E−16; RPL37, |
| | −6.0E+00, 4.7E−11; CEBPZ, −6.0E+00, 5.1E−11; UBA52, −6.0E+00, 2.6E−13; PSMA7, −5.9E+00, 2.8E−13; CCT6A, −5.9E+00, 9.9E−11; HMGN1, −5.9E+00, 4.3E−12; |
| | DNAJA1, −5.9E+00, 3.3E−12; HNRNPA3, −5.8E+00, 2.2E−10; SSRP1, −5.8E+00, 6.4E−14; VCP, −5.8E+00, 3.8E−11; BTF3, −5.8E+00, 1.9E−13; RPL38, |
| | −5.8E+00, 8.3E−12; SUMO2, −5.7E+00, 1.9E−12; IDH1, −5.7E+00, 2.9E−10; H1-2, −5.7E+00, 3.1E−10; IPO5, −5.7E+00, 8.8E−10; NONO, −5.7E+00, 3.6E−10; |
| | AZIN1, −5.7E+00, 4.0E−12; PTGES3, −5.7E+00, 1.3E−09; CCT2, −5.7E+00, 2.1E−10; NAP1L1, −5.7E+00, 6.0E−12; RPL36AL, −5.6E+00, 1.1E−16; BEX3, |
| | −5.6E+00, 2.9E−13; RPL9, −5.6E+00, 7.5E−10; YWHAQ, −5.6E+00, 1.4E−09; HNRNPH3, −5.6E+00, 1.4E−10; DPPA4, −5.6E+00, 4.2E−10; HNRNPAB, −5.6E+00, |
| | 1.5E−12; RPS29, −5.5E+00, 1.4E−09; PARK7, −5.5E+00, 3.2E−12; SRP14, −5.5E+00, 9.4E−12; UCHL1, −5.5E+00, 2.7E−09; TPM4, −5.5E+00, 3.0E−09; CCT8, |
| | −5.4E+00, 2.7E−10; MSH6, −5.4E+00, 2.2E−09; CBX1, −5.4E+00, 2.2E−11; SOX2, −5.4E+00, 3.0E−10; SKP1, −5.4E+00, 5.2E−11; COX4I1, −5.4E+00, 9.1E−12; |
| | CHD4, −5.4E+00, 1.4E−08; AKIRIN1, −5.4E+00, 5.2E−09; SOX4, −5.4E+00, 9.5E−12; SMC3, −5.3E+00, 3.4E−09; HNRNPM, −5.3E+00, 1.6E−08; TMED2, |
| | −5.3E+00, 6.5E−14; C11orf58, −5.3E+00, 6.2E−10; COX7C, −5.3E+00, 6.7E−10; CENPF, −5.3E+00, 3.8E−09; PDIA6, −5.3E+00, 7.0E−09; IFITM1, −5.3E+00, |
| | 7.7E−15; CNBP, −5.3E+00, 6.6E−10; G3BP1, −5.3E+00, 1.7E−08; PRDX6, −5.2E+00, 1.9E−09; DHCR24, −5.2E+00, 8.7E−09; ADH5, −5.2E+00, 4.8E−10; CYCS, |
| | −5.2E+00, 1.0E−10; DBI, −5.2E+00, 2.8E−13; UQCRH, −5.2E+00, 2.1E−09; MCM4, −5.2E+00, 1.2E−08; PMAIP1, −5.2E+00, 2.6E−12; DDX21, −5.2E+00, 7.2E−09; |
| | HIST1H1E, −5.2E+00, 5.4E−09; YWHAB, −5.2E+00, 1.2E−08; KPNA2, −5.1E+00, 1.7E−08; MTHFD2, −5.1E+00, 3.4E−10; DEK, −5.1E+00, 4.6E−08; |
| | IGF2BP1, −5.1E+00, 1.6E−08; XRCC5, −5.1E+00, 2.9E−09; PDIA4, −5.1E+00, 8.3E−10; PAICS, −5.1E+00, 4.6E−08; CSE1L, −5.1E+00, 1.1E−07; RHOA, −5.1E+00, |
| | 4.0E−09; HMGCS1, −5.1E+00, 1.9E−07; TMEM167A, −5.1E+00, 5.9E−09; DYNLL1, −5.1E+00, 1.3E−09; SLC25A3, −5.1E+00, 5.8E−10; IQGAP1, −5.0E+00, |
| | 1.1E−07; PSIP1, −5.0E+00, 8.3E−08; RPL37A, −5.0E+00, 8.4E−08; FKBP3, −5.0E+00, 1.7E−09; CLDN6, −5.0E+00, 5.6E−10; CD63, −5.0E+00, 9.7E−12; RPS25, |
| | −5.0E+00, 2.2E−10; ZNF770, −5.0E+00, 4.0E−09; PTBP1, −4.9E+00, 3.1E−09; SUPT16H, −4.9E+00, 2.4E−07; H2AC17, −4.9E+00, 1.3E−09; SKIL, −4.9E+00, |
| | 8.9E−07; TPR, −4.9E+00, 2.6E−07; UQCRB, −4.9E+00, 7.7E−11; PKM, −4.8E+00, 1.4E−07; HMGB3, −4.8E+00, 1.1E−08; SERPINB9, −4.8E+00, 2.2E−09; |
| | ARL6IP1, −4.8E+00, 8.9E−09; HNRNPL, −4.8E+00, 6.0E−09; CACYBP, −4.8E+00, 5.0E−12; RPS26, −4.8E+00, 2.9E−09; RBMX, −4.8E+00, 2.9E−07; STIP1, |
| | −4.8E+00, 1.5E−08; RSL1D1, −4.8E+00, 2.1E−08; APEX1, −4.8E+00, 3.7E−10; MDH1, −4.8E+00, 9.3E−09; CBX3, −4.7E+00, 9.2E−08; CAPRIN1, −4.7E+00, 5.8E−07; |
| | SLC16A1, −4.7E+00, 9.5E−07; PA2G4, −4.7E+00, 1.5E−08; EPRS1, −4.7E+00, 3.5E−07; EIF5A, −4.7E+00, 6.5E−09; H3C2, −4.7E+00, 4.4E−09; CNN3, |
| | −4.7E+00, 8.8E−08; DDX5, −4.7E+00, 1.3E−06; SNHG5, −4.7E+00, 1.5E−09; G3BP2, −4.6E+00, 6.3E−07; CCDC47, −4.6E+00, 5.0E−10; HNRNPDL, −4.6E+00, |
| | 1.7E−06; EIF1, −4.6E+00, 2.8E−09; SMARCA5, −4.6E+00, 2.3E−06; CAP1, −4.6E+00, 7.1E−09; PLS3, −4.6E+00, 3.3E−06; CLTC, −4.6E+00, 9.9E−07; H3C4, |
| | −4.6E+00, 1.1E−09; EIF5B, −4.6E+00, 2.5E−06; ANXA2, −4.6E+00, 4.7E−07; RCC2, −4.6E+00, 1.7E−06; TARDBP, −4.6E+00, 4.6E−08; KPNB1, −4.6E+00, 2.3E−06; |
| | RAD21, −4.5E+00, 5.8E−07; H2AC20, −4.5E+00, 1.7E−08; HSPH1, −4.5E+00, 5.0E−07; SLIRP, −4.5E+00, 9.6E−09; DDX1, −4.5E+00, 3.0E−07; MATR3, |
| | −4.5E+00, 1.5E−06; ANP32E, −4.5E+00, 1.9E−07; UBC, −4.4E+00, 1.3E−07; ZFAND5, −4.4E+00, 2.2E−07; HDLBP, −4.4E+00, 4.7E−06; EIF4G1, −4.4E+00, |
| | 1.6E−06; SRSF7, −4.4E+00, 2.5E−07; C14orf166, −4.4E+00, 1.4E−08; NAA50, −4.4E+00, 2.6E−06; EIF3E, −4.4E+00, 6.4E−06; PRPF40A, −4.4E+00, 5.6E−06; DSG2, |
| | −4.4E+00, 1.9E−06; NAE1, −4.4E+00, 2.4E−08; EIF4A2, −4.4E+00, 6.0E−06; METAP2, −4.3E+00, 4.9E−07; YBX3, −4.3E+00, 1.8E−08; ANP32B, −4.3E+00, 6.8E−08; |
| | SERPINH1, −4.3E+00, 2.1E−07; TXNRD1, −4.3E+00, 5.3E−06; SUB1, −4.3E+00, 4.5E−08; EIF3A, −4.3E+00, 9.1E−06; EIF2S2, −4.3E+00, 3.6E−07; ZFP42, |
| | −4.3E+00, 1.7E−06; MRFAP1, −4.3E+00, 1.7E−08; XRCC6, −4.3E+00, 1.5E−05; SEPTIN2, −4.3E+00, 9.3E−06; LDHA, −4.3E+00, 9.6E−10; EIF5, −4.3E+00, 3.5E−06; |
| | NARS1, −4.2E+00, 1.5E−06; HSPA9, −4.2E+00, 1.1E−05; RPL22, −4.2E+00, 3.3E−07; USP44, −4.2E+00, 1.4E−05; FKBP4, −4.2E+00, 3.8E−09; MFGE8, |
| | −4.2E+00, 1.5E−05; ATP5PD, −4.2E+00, 1.9E−09; HINT1, −4.2E+00, 2.0E−08; PPM1G, −4.2E+00, 6.7E−07; SERP1, −4.2E+00, 3.8E−09; OAZ1, −4.2E+00, 8.3E−08; |
| | HNRNPR, −4.2E+00, 1.0E−05; DDX3X, −4.2E+00, 6.5E−06; CCNG1, −4.2E+00, 1.0E−06; ARPC5, −4.2E+00, 6.1E−09; NDUFA4, −4.2E+00, 2.4E−07; DDX6, |
| | −4.1E+00, 2.2E−05; PSMD2, −4.1E+00, 1.1E−08; CLIC1, −4.1E+00, 5.1E−10; MAPRE1, −4.1E+00, 8.8E−08; EID1, −4.1E+00, 2.2E−07; NOL11, −4.1E+00, |
| | 1.0E−07; GDI2, −4.1E+00, 3.1E−05; ATP5PO, −4.1E+00, 1.5E−07; EIF3I, −4.1E+00, 3.0E−10; ST13, −4.0E+00, 3.7E−06; TFAM, −4.0E+00, 8.1E−08; BSG, −4.0E+00, |
| | 7.9E−07; PHB2, −4.0E+00, 2.7E−10; HIF1A, −4.0E+00, 8.9E−06; CCT7, −4.0E+00, 2.1E−07; DSTN, −4.0E+00, 4.4E−06; SRSF1, −4.0E+00, 3.0E−05; PEG10, |
| | −4.0E+00, 2.4E−07; ND5, −4.0E+00, 9.9E−15; MLEC, −4.0E+00, 6.7E−06; TMBIM6, −4.0E+00, 9.1E−06; RPL23A, −4.0E+00, 1.6E−06; EIF3M, −4.0E+00, |
| | 1.2E−06; SNRPD2, −4.0E+00, 1.3E−07; CLIC4, −3.9E+00, 1.2E−04; NFE2L1, −3.9E+00, 1.3E−06; CCNI, −3.9E+00, 2.7E−06; ACLY, −3.9E+00, 6.7E−05; NUDT21, |
| | −3.9E+00, 1.2E−06; PHB, −3.9E+00, 8.6E−09; SON, −3.9E+00, 1.1E−04; H2AC14, −3.9E+00, 1.3E−09; ZC3H15, −3.9E+00, 3.7E−06; HMGCR, −3.9E+00, |
| | 1.0E−04; TOP2A, −3.9E+00, 2.7E−04; MRPL51, −3.9E+00, 2.5E−10; SELENOW, −3.9E+00, 1.6E−08; TFRC, −3.8E+00, 7.9E−06; TXN, −3.8E+00, 1.2E−06; YWHAG, |
| | −3.8E+00, 7.2E−05; ARCN1, −3.8E+00, 1.2E−07; CDC42, −3.8E+00, 2.2E−06; PPP1CC, −3.8E+00, 3.3E−05; LRRC75A-AS1, −3.8E+00, 4.8E−06; SNRPD3, |
| | −3.8E+00, 2.3E−06; HDAC2, −3.8E+00, 1.5E−04; ATP5PB, −3.8E+00, 2.1E−07; ACTR2, −3.8E+00, 3.6E−05; STRAP, −3.8E+00, 5.0E−06; GNPTAB, −3.8E+00, |
| | 1.4E−05; ALDOA, −3.8E+00, 2.3E−06; ADAR, −3.8E+00, 8.2E−05; ZNF217, −3.8E+00, 4.6E−05; COX5B, −3.8E+00, 4.4E−07; SNRPF, −3.8E+00, 4.2E−08; GNL3, |
| | −3.8E+00, 7.0E−06; TERF1, −3.8E+00, 6.8E−05; DNMT3B, −3.7E+00, 1.5E−06; TCOF1, −3.7E+00, 1.2E−05; AP2M1, −3.7E+00, 7.2E−07; MARCKSL1, −3.7E+00, |
| | 8.3E−07; ERH, −3.7E+00, 1.2E−06; CDH1, −3.7E+00, 2.5E−04; ILF2, −3.7E+00, 1.7E−06; ATP6V1G1, −3.7E+00, 1.6E−08; NDUFA13, −3.6E+00, 3.6E−07; |
| | FSCN1, −3.6E+00, 3.6E−07; USP9X, −3.6E+00, 2.8E−07; SPARC, −3.6E+00, 4.7E−07; THRAP3, −3.6E+00, 3.1E−04; SNRNP200, −3.6E+00, 4.3E−05; PGK1, |
| | −3.6E+00, 4.8E−06; CCND1, −3.6E+00, 3.0E−06; SMARCE1, −3.6E+00, 1.2E−05; NREP, −3.6E+00, 4.6E−05; FUS, −3.6E+00, 5.0E−04; LMNB1, −3.6E+00, |
| | 1.7E−04; MORF4L2, −3.6E+00, 9.3E−05; SRSF6, −3.6E+00, 6.3E−06; NOP56, −3.6E+00, 3.7E−05; CALU, −3.6E+00, 1.6E−04; MKI67, −3.6E+00, 8.9E−04; ND4L, |
| | −3.6E+00, 2.4E−04; SRSF10, −3.6E+00, 3.3E−04; H4C5, −3.6E+00, 6.3E−07; SF3B2, −3.6E+00, 3.1E−05; EIF4B, −3.6E+00, 7.6E−04; BZW1, −3.6E+00, 6.8E−06; |
| | SOD1, −3.6E+00, 5.2E−07; ABCF1, −3.5E+00, 8.0E−06; COX1, −3.5E+00, 7.9E−19; PCNP, −3.5E+00, 3.3E−05; SF3A3, −3.5E+00, 5.3E−06; SYNCRIP, −3.5E+00, |
| | 7.8E−04; DDX18, −3.5E+00, 3.8E−05; PODXL, −3.5E+00, 2.1E−04; IDI1, −3.5E+00, 9.2E−06; PAIP2, −3.5E+00, 1.7E−05; ARPC3, −3.5E+00, 1.7E−07; ACAT2, |
| | −3.5E+00, 5.1E−05; HNRNPAO, −3.5E+00, 9.3E−07; REST, −3.5E+00, 8.6E−04; DNTTIP2, −3.4E+00, 6.4E−06; SSR3, −3.4E+00, 3.0E−05; AP1S2, −3.4E+00, |
| | 8.5E−04; PCBP2, −3.4E+00, 7.6E−04; LAPTM4A, −3.4E+00, 6.1E−06; PSMD8, −3.4E+00, 1.4E−07; SRRM1, −3.4E+00, 4.4E−04; NUTF2, −3.4E+00, 2.7E−07; |
| | TOP2B, −3.4E+00, 1.2E−03; COX8A, −3.4E+00, 5.4E−08; PPIG, −3.4E+00, 5.2E−04; EIF3F, −3.4E+00, 6.0E−07; KARS1, −3.4E+00, 1.8E−05; LUC7L3, −3.4E+00, |
| | 8.8E−04; IMPDH2, −3.4E+00, 6.5E−06; TOMM20, −3.4E+00, 1.3E−04; ATP5MC3, −3.4E+00, 1.0E−05; KDELR1, −3.4E+00, 1.3E−08; BAZ1B, −3.4E+00, 6.8E−04; |
| | STAU1, −3.4E+00, 2.0E−04; CNN2, −3.4E+00, 5.3E−05; PSAP, −3.4E+00, 2.2E−04; FAM136A, −3.4E+00, 5.3E−06; APOE, −3.4E+00, 5.9E−06; TRIM28, |
| | −3.4E+00, 1.1E−04; EDF1, −3.4E+00, 1.3E−05; NOLC1, −3.4E+00, 2.3E−04; MGST1, −3.3E+00, 1.9E−04; CNMD, −3.3E+00, 5.8E−04; ATP5MG, −3.3E+00, |
| | 1.9E−07; BMS1, −3.3E+00, 5.7E−05; CAND1, −3.3E+00, 9.4E−04; ATXN7L3B, −3.3E+00, 2.6E−07; RANBP1, −3.3E+00, 1.5E−05; H2AC8, −3.3E+00, 9.4E−07; SRP72, |
| | −3.3E+00, 2.7E−04; PAPOLA, −3.3E+00, 1.7E−03; RDX, −3.3E+00, 4.4E−04; ATP5IF1, −3.3E+00, 1.6E−06; PSMD12, −3.3E+00, 1.3E−04; MT1G, −3.3E+00, |
| | 1.6E−12; ENSA, −3.3E+00, 3.2E−05; ETF1, −3.3E+00, 2.8E−04; GLO1, −3.3E+00, 3.5E−04; DENR, −3.3E+00, 7.6E−04; DLGAP5, −3.3E+00, 7.7E−04; PRRC2C, |
| | −3.3E+00, 5.2E−04; MAP1B, −3.2E+00, 3.2E−03; ADSS, −3.2E+00, 1.7E−04; RBM3, −3.2E+00, 2.0E−05; SNRPC, −3.2E+00, 2.5E−06; NUDT15, −3.2E+00, 4.3E−06; |
| | PSMA3, −3.2E+00, 7.6E−05; PLAAT3, −3.2E+00, 1.4E−05; PPP2CA, −3.2E+00, 4.6E−04; CTNNB1, −3.2E+00, 2.6E−03; ANP32A, −3.2E+00, 1.5E−03; |
| | HMGN2, −3.2E+00, 1.8E−04; UQCR10, −3.2E+00, 1.3E−07; PABPC4, −3.2E+00, 1.2E−05; NRAS, −3.2E+00, 7.5E−06; TOMM7, −3.2E+00, 3.8E−07; |
| | LOC105377901, −3.2E+00, 5.2E−03; PSMB1, −3.2E+00, 6.9E−05; PGD, −3.2E+00, 8.2E−05; SSBP1, −3.2E+00, 1.7E−04; EBNA1BP2, −3.2E+00, 4.0E−08; |
| | SPCS3, −3.2E+00, 1.5E−05; PSMA2, −3.2E+00, 1.8E−04; MRPL3, −3.2E+00, 1.0E−03; VCAN, −3.1E+00, 4.0E−04; GNAS, −3.1E+00, 3.9E−03; TMED10, |
| | −3.1E+00, 9.8E−05; HNRNPD, −3.1E+00, 3.7E−03; LASP1, −3.1E+00, 8.0E−05; FABP5, −3.1E+00, 4.0E−07; DSP, −3.1E+00, 2.9E−03; APLP2, −3.1E+00, 4.6E−03; |
| | KRAS, −3.1E+00, 4.7E−04; LSM4, −3.1E+00, 8.5E−07; DPYSL3, −3.1E+00, 6.6E−03; FLNA, −3.1E+00, 4.6E−03; DKC1, −3.1E+00, 3.5E−05; FDFT1, |
| | −3.1E+00, 3.6E−03; HIST1H2AC, −3.1E+00, 2.8E−06; ATF4, −3.1E+00, 2.5E−04; MARCKS, −3.1E+00, 2.2E−05; DNAJC8, −3.1E+00, 4.5E−05; GPX4, −3.1E+00, |
| | 2.2E−07; SMARCD1, −3.1E+00, 3.4E−06; PSMB4, −3.1E+00, 4.9E−06; CDCA7L, −3.0E+00, 1.1E−04; COX6B1, −3.0E+00, 1.2E−05; PDPN, −3.0E+00, 2.7E−04; |
| | FBL, −3.0E+00, 1.9E−04; FAM60A, −3.0E+00, 6.6E−03; DHX9, −3.0E+00, 6.7E−03; SMC4, −3.0E+00, 6.6E−03; GSPT1, −3.0E+00, 2.5E−04; NDUFB8, −3.0E+00, |
| | 7.7E−07; BCCIP, −3.0E+00, 1.5E−04; PSMD11, −3.0E+00, 5.8E−04; DDX3Y, −3.0E+00, 2.1E−04; HSPE1, −3.0E+00, 5.1E−06; SHFM1, −3.0E+00, 1.5E−04; |
| | ARMT1, −3.0E+00, 1.8E−04; ENY2, −3.0E+00, 3.4E−05; CLTA, −3.0E+00, 1.8E−04; SAP18, −3.0E+00, 1.1E−05; NOL7, −3.0E+00, 1.0E−05; HIST1H2BC, |
| | −3.0E+00, 4.1E−06; TSPAN6, −3.0E+00, 4.6E−06; SLC38A2, −3.0E+00, 5.2E−03; ABRACL, −3.0E+00, 8.9E−06; MCM7, −3.0E+00, 3.5E−05; MTHFD1, −2.9E+00, |
| | 4.1E−04; RARS, −2.9E+00, 5.7E−04; TOMM6, −2.9E+00, 4.3E−07; UGP2, −2.9E+00, 1.8E−03; NR6A1, −2.9E+00, 9.5E−03; LARS, −2.9E+00, 8.6E−03; UBE2V2, |
| | −2.9E+00, 2.4E−04; VDAC1, −2.9E+00, 2.7E−04; ID1, −2.9E+00, 4.8E−06; ACVR2B, −2.9E+00, 4.1E−03; RFC1, −2.9E+00, 2.9E−03; DBN1, −2.9E+00, 7.0E−04; |
| | USP7, −2.9E+00, 3.4E−03; TALDO1, −2.9E+00, 1.6E−05; CD81, −2.9E+00, 4.2E−06; SBNO1, −2.9E+00, 4.0E−03; PRDX3, −2.9E+00, 3.4E−05; MPHOSPH10, |
| | −2.9E+00, 7.2E−06; PSMA6, −2.9E+00, 2.0E−04; PCBP1, −2.9E+00, 1.9E−04; NUFIP2, −2.9E+00, 1.0E−02; RRM1, −2.9E+00, 3.9E−05; RPL13AP5, −2.9E+00, |
| | 1.2E−05; RIF1, −2.9E+00, 8.2E−03; STAG2, −2.9E+00, 1.2E−02; HIST1H2AG, −2.9E+00, 1.8E−04; WDR1, −2.9E+00, 1.2E−03; KHDRBS1, −2.9E+00, 5.9E−03; |
| | TRAM1, −2.8E+00, 1.2E−04; COX3, −2.8E+00, 3.6E−09; DDX24, −2.8E+00, 3.3E−04; GOT2, −2.8E+00, 3.4E−04; DDX17, −2.8E+00, 1.1E−02; MYL12B, |
| | −2.8E+00, 2.3E−05; CCNA2, −2.8E+00, 1.5E−04; PFDN5, −2.8E+00, 1.7E−05; EIF1AX, −2.8E+00, 8.7E−05; PNRC2, −2.8E+00, 9.5E−05; SQLE, −2.8E+00, 4.5E−03; |
| | CENPU, −2.8E+00, 2.4E−03; DNAJC10, −2.8E+00, 3.1E−03; SNHG16, −2.8E+00, 3.3E−04; SAFB, −2.8E+00, 1.7E−03; ELP2, −2.8E+00, 1.2E−03; C6orf62, |
| | −2.8E+00, 5.2E−04; RTF1, −2.8E+00, 4.8E−04; PAFAH1B2, −2.8E+00, 2.9E−03; TMA7, −2.8E+00, 6.5E−06; MRPS21, −2.8E+00, 5.7E−05; NSA2, −2.8E+00, |
| | 2.1E−05; PCNA, −2.8E+00, 2.0E−04; GRSF1, −2.8E+00, 5.7E−04; HNRNPH1, −2.8E+00, 1.6E−02; CWC15, −2.8E+00, 2.2E−06; KNOP1, −2.8E+00, 4.8E−04; |
| | C21orf59, −2.7E+00, 1.9E−04; SCG3, −2.7E+00, 9.2E−05; PPDPF, −2.7E+00, 8.7E−06; UBE2I, −2.7E+00, 7.6E−04; TMPO, −2.7E+00, 1.2E−02; SPTAN1, |
| | −2.7E+00, 1.4E−02; NCBP2, −2.7E+00, 2.3E−04; FRAT2, −2.7E+00, 5.2E−08; VMA21, −2.7E+00, 6.7E−06; SOX11, −2.7E+00, 5.6E−04; RBM12, −2.7E+00, 9.2E−04; |
| | ZNF483, −2.7E+00, 5.8E−03; MRPS34, −2.7E+00, 6.2E−05; USP1, −2.7E+00, 2.7E−03; PPP2R1B, −2.7E+00, 1.3E−04; PSMD7, −2.7E+00, 8.7E−04; LITAF, |
| | −2.7E+00, 9.7E−03; SNRPD1, −2.7E+00, 1.1E−03; RMND5A, −2.7E+00, 4.0E−04; MAGED2, −2.7E+00, 8.3E−05; TCEAL4, −2.7E+00, 6.9E−05; TUBA1A, |
| | −2.7E+00, 1.9E−04; ROR1, −2.7E+00, 1.9E−02; MDH2, −2.7E+00, 1.8E−05; UGDH, −2.7E+00, 5.9E−04; NDUFB9, −2.7E+00, 4.5E−04; TPX2, −2.7E+00, 7.1E−03; |
| | RBBP7, −2.7E+00, 1.2E−03; SUMO1, −2.6E+00, 1.9E−03; RPL41, −2.6E+00, 3.1E−04; GOLGB1, −2.6E+00, 1.7E−02; ERP29, −2.6E+00, 1.4E−04; SNRPE, |
| | −2.6E+00, 7.2E−04; MRPL19, −2.6E+00, 6.9E−05; PCDH7, −2.6E+00, 3.4E−02; PSMC6, −2.6E+00, 2.7E−04; RAD50, −2.6E+00, 1.4E−02; CTR9, −2.6E+00, 2.8E−04; |
| | YME1L1, −2.6E+00, 1.4E−02; PSMC3, −2.6E+00, 9.9E−05; ARPC2, −2.6E+00, 9.3E−03; PSME3, −2.6E+00, 4.9E−04; PDHA1, −2.6E+00, 3.0E−06; TOMM70, |
| | −2.6E+00, 1.1E−03; EIF3K, −2.6E+00, 1.2E−03; HYOU1, −2.6E+00, 3.0E−03; IPO7, −2.6E+00, 3.1E−02; NES, −2.6E+00, 5.2E−03; ND6, −2.6E+00, 1.2E−02; |
| | PWARSN, −2.6E+00, 3.0E−02; DNAJC2, −2.6E+00, 6.4E−04; FZD7, −2.6E+00, 3.8E−05; LMAN1, −2.6E+00, 5.7E−03; SUMO3, −2.6E+00, 2.0E−06; PRMT1, |
| | −2.6E+00, 1.0E−03; GPI, −2.6E+00, 2.0E−03; GADD45GIP1, −2.6E+00, 3.3E−07; MRPS16, −2.6E+00, 1.3E−05; BUB3, −2.6E+00, 3.9E−04; SEC61B, −2.6E+00, |
| | 2.2E−04; DCP2, −2.6E+00, 2.7E−02; PSMC2, −2.6E+00, 6.9E−05; HCFC1, −2.6E+00, 5.5E−04; CHCHD2, −2.6E+00, 9.6E−05; ND4, −2.6E+00, 2.2E−11; PRRC2A, |
| | −2.6E+00, 6.4E−03; TMEM59, −2.6E+00, 7.7E−06; CKS2, −2.6E+00, 4.3E−04; DNAJC7, −2.6E+00, 3.9E−03; DUT, −2.6E+00, 1.9E−03; ZNF146, −2.6E+00, |
| | 1.9E−02; TUBB2B, −2.6E+00, 9.9E−05; SIRT1, −2.6E+00, 9.8E−03; MED1, −2.5E+00, 1.5E−03; FLT1, −2.5E+00, 9.7E−03; RAD23B, −2.5E+00, 2.2E−02; CXADR, |
| | −2.5E+00, 3.2E−02; MCM6, −2.5E+00, 1.8E−02; GMFB, −2.5E+00, 4.1E−04; PPP2R2B, −2.5E+00, 3.4E−02; HSBP1, −2.5E+00, 3.2E−04; RAC1, −2.5E+00, 8.3E−04; |
| | CKB, −2.5E+00, 6.5E−04; ATP5C1, −2.5E+00, 7.4E−04; SPDL1, −2.5E+00, 6.5E−04; EIF1B, −2.5E+00, 7.5E−05; CDV3, −2.5E+00, 2.4E−03; TRIM71, |
| | −2.5E+00, 6.7E−03; SALL4, −2.5E+00, 3.2E−02; F11R, −2.5E+00, 9.8E−03; GPX1, −2.5E+00, 1.0E−05; MCM3, −2.5E+00, 1.6E−02; TRMT10C, −2.5E+00, 3.0E−05; |
| | ARF1, −2.5E+00, 1.5E−03; TIMM13, −2.5E+00, 4.4E−05; LINC00678, −2.5E+00, 8.5E−05; ATP8, −2.5E+00, 3.9E−02; MYH9, −2.5E+00, 2.5E−02; PDCD5, |
| | −2.5E+00, 7.5E−05; PPAT, −2.5E+00, 7.2E−03; BAZ2A, −2.5E+00, 5.3E−03; ATP6VOC, −2.5E+00, 2.2E−06; XPO1, −2.5E+00, 2.0E−02; ACTR3, −2.5E+00, |
| | 1.5E−02; NRBP1, −2.5E+00, 4.4E−04; UBTF, −2.5E+00, 8.7E−04; COX5A, −2.5E+00, 9.8E−04; DDX52, −2.5E+00, 7.5E−05; PRDX5, −2.5E+00, 5.9E−05; MAP1LC3B, |
| | −2.5E+00, 1.0E−05; DAZAP1, −2.5E+00, 3.2E−03; TROVE2, −2.5E+00, 9.9E−03; FXR1, −2.5E+00, 4.6E−02; TPM1, −2.5E+00, 3.3E−02; DDB1, −2.5E+00, 4.4E−03; |
| | TCEB2, −2.5E+00, 2.0E−04; CYTB, −2.5E+00, 2.6E−05; TFDP2, −2.4E+00, 3.9E−02; PSMD4, −2.4E+00, 1.1E−03; HIST1H3E, −2.4E+00, 6.8E−05; EIF2AK1, |
| | −2.4E+00, 1.2E−03; AES, −2.4E+00, 1.6E−05; XPOT, −2.4E+00, 1.8E−02; TAX1BP1, −2.4E+00, 1.9E−02; C9orf78, −2.4E+00, 8.3E−05; PFN2, −2.4E+00, 5.6E−04; |
| | FAM98A, −2.4E+00, 7.9E−04; RRM2, −2.4E+00, 1.9E−03; ITGA6, −2.4E+00, 3.0E−02; AURKAIP1, −2.4E+00, 5.9E−06; TMEM97, −2.4E+00, 3.2E−03; |
| | PSMB6, −2.4E+00, 3.1E−04; MOB1A, −2.4E+00, 3.8E−03; PPA1, −2.4E+00, 2.8E−03; ABCE1, −2.4E+00, 1.0E−02; PRR13, −2.4E+00, 4.8E−06; POLR2L, |
| | −2.4E+00, 4.4E−05; VDAC2, −2.4E+00, 1.8E−03; VBP1, −2.4E+00, 1.9E−04; SRSF5, −2.4E+00, 6.1E−03; PRKAR1A, −2.4E+00, 4.0E−03; UBLCP1, −2.4E+00, |
| | 2.9E−04; ATP51, −2.4E+00, 4.5E−06; IK, −2.4E+00, 1.9E−04; KIF5B, −2.4E+00, 3.3E−02; SRSF2, −2.4E+00, 4.4E−03; ATP5G2, −2.4E+00, 9.6E−04; UBXN7, |
| | −2.4E+00, 2.2E−02; RAB13, −2.4E+00, 1.7E−04; 4.4E+04, −2.4E+00, 4.4E−02; MAD2L2, −2.4E+00, 8.8E−04; VPS29, −2.4E+00, 2.6E−04; RCN2, −2.4E+00, |
| | 1.5E−02; VDAC3, −2.4E+00, 1.5E−04; KHSRP, −2.4E+00, 7.7E−03; LTA4H, −2.4E+00, 2.2E−03; SUDS3, −2.4E+00, 3.3E−03; PERP, −2.4E+00, 4.2E−04; CCND2, |
| | −2.4E+00, 1.3E−02; COX6A1, −2.4E+00, 7.1E−04; METTL5, −2.4E+00, 2.3E−05; SRP54, −2.4E+00, 4.3E−03; RBM27, −2.4E+00, 3.7E−02; UBA2, −2.4E+00, 2.5E−02; |
| | GID8, −2.4E+00, 1.4E−04; NME1, −2.3E+00, 1.8E−06; SARS, −2.3E+00, 2.5E−04; UQCR11, −2.3E+00, 3.6E−05; CPSF2, −2.3E+00, 2.2E−02; UTP11, |
| | −2.3E+00, 1.3E−04; GNAI3, −2.3E+00, 6.0E−03; ATP1A1, −2.3E+00, 1.9E−02; HADHA, −2.3E+00, 1.0E−02; SRP9, −2.3E+00, 1.3E−02; TCEAL9, −2.3E+00, 2.9E−06; |
| | CLSPN, −2.3E+00, 1.3E−02; ND2, −2.3E+00, 5.8E−03; TPM2, −2.3E+00, 2.5E−03; SNRPA1, −2.3E+00, 1.8E−03; MRPL57, −2.3E+00, 1.8E−04; GHITM, |
| | −2.3E+00, 4.9E−04; CAPZA1, −2.3E+00, 3.3E−02; PCDH18, −2.3E+00, 1.8E−03; XPO5, −2.3E+00, 7.7E−04; PSMC5, −2.3E+00, 7.9E−04; MIS18A, −2.3E+00, |
| | 1.3E−04; ZNF460, −2.3E+00, 3.0E−03; CCNB1, −2.3E+00, 3.0E−02; GART, −2.3E+00, 2.9E−02; DNMT3A, −2.3E+00, 2.6E−02; RPN1, −2.3E+00, 3.8E−03; PDHB, |
| | −2.3E+00, 2.0E−03; PTTG1, −2.3E+00, 8.8E−05; ALDH7A1, −2.3E+00, 3.3E−02; PRPF4B, −2.3E+00, 3.7E−02; WBP11, −2.3E+00, 2.3E−03; DHX33, −2.3E+00, |
| | 1.1E−03; NFYB, −2.3E+00, 2.6E−03; SMC2, −2.3E+00, 2.2E−02; MDM2, −2.3E+00, 4.8E−02; FAM213A, −2.3E+00, 9.4E−04; TUBB4B, −2.3E+00, 7.5E−04; |
| | OSTC, −2.3E+00, 5.1E−03; LSM3, −2.3E+00, 8.2E−04; TAF7, −2.3E+00, 5.4E−03; PRDM14, −2.3E+00, 3.9E−02; CCNC, −2.3E+00, 2.0E−03; RMI1, −2.3E+00, |
| | 1.3E−03; PHGDH, −2.3E+00, 2.0E−02; DIS3, −2.3E+00, 1.9E−03; CLU, −2.3E+00, 1.3E−02; GRB2, −2.3E+00, 3.1E−02; BRD3, −2.3E+00, 2.1E−03; RALBP1, |
| | −2.3E+00, 2.2E−03; TIMELESS, −2.3E+00, 3.9E−03; ZCRB1, −2.3E+00, 5.1E−05; CNOT7, −2.3E+00, 5.4E−03; ODC1, −2.3E+00, 5.4E−04; COX7B, −2.2E+00, |
| | 2.9E−04; CUTA, −2.2E+00, 1.1E−04; ND1, −2.2E+00, 6.8E−03; RNF20, −2.2E+00, 2.4E−03; IPW, −2.2E+00, 1.0E−02; ITM2B, −2.2E+00, 6.1E−03; RNF7, |
| | −2.2E+00, 1.8E−04; MCL1, −2.2E+00, 3.2E−02; PTP4A1, −2.2E+00, 6.6E−03; PTP4A2, −2.2E+00, 7.0E−03; CD164, −2.2E+00, 2.3E−03; ZMAT2, −2.2E+00, 2.9E−04; |
| | CASP8AP2, −2.2E+00, 3.4E−02; UQCRC1, −2.2E+00, 6.1E−04; IFITM3, −2.2E+00, 3.0E−05; MAD2L1, −2.2E+00, 4.5E−04; MSN, −2.2E+00, 9.8E−03; |
| | COX7A2, −2.2E+00, 6.2E−03; EIF4EBP2, −2.2E+00, 3.4E−02; ATP5J, −2.2E+00, 2.7E−03; TSR1, −2.2E+00, 2.0E−03; PRIM1, −2.2E+00, 7.4E−04; FERMT2, |
| | −2.2E+00, 2.2E−02; ATP6, −2.2E+00, 9.1E−05; HN1L, −2.2E+00, 4.4E−02; DDX50, −2.2E+00, 7.0E−03; COPA, −2.2E+00, 2.3E−02; GTF2F1, −2.2E+00, 1.5E−04; |
| | ARRDC3, −2.2E+00, 5.0E−04; CNIH4, −2.2E+00, 7.0E−03; HN1, −2.2E+00, 4.1E−03; H2AFV, −2.2E+00, 1.9E−02; ATP1B3, −2.2E+00, 3.0E−02; |
| | LOC107985690, −2.2E+00, 4.9E−04; CDK2AP1, −2.2E+00, 1.2E−04; SLC2A3, −2.2E+00, 2.5E−02; HMMR, −2.2E+00, 2.1E−02; HSPA13, −2.2E+00, 1.4E−04; |
| | GNPDA1, −2.2E+00, 9.8E−04; CDC6, −2.2E+00, 5.5E−03; COPS2, −2.2E+00, 5.1E−03; ROMO1, −2.2E+00, 1.2E−04; CKS1B, −2.2E+00, 1.0E−05; ARL14EPL, |
| | −2.2E+00, 3.0E−05; SLC25A5, −2.1E+00, 7.4E−03; MRPL12, −2.1E+00, 8.8E−04; NIFK, −2.1E+00, 8.1E−04; PRKAR2A, −2.1E+00, 2.5E−03; LSM7, −2.1E+00, |
| | 7.7E−04; ASF1A, −2.1E+00, 2.4E−05; OIP5-AS1, −2.1E+00, 1.9E−02; PTTG1IP, −2.1E+00, 1.3E−02; HIST1H2AH, −2.1E+00, 1.8E−04; KAT7, −2.1E+00, 3.5E−03; |
| | C1QBP, −2.1E+00, 6.1E−03; HIST1H2AL, −2.1E+00, 1.0E−03; VRTN, −2.1E+00, 1.7E−03; RRP15, −2.1E+00, 8.8E−03; HIST1H4D, −2.1E+00, 6.8E−05; |
| | RAB8A, −2.1E+00, 4.0E−04; KDR, −2.1E+00, 3.6E−03; AIF1L, −2.1E+00, 2.1E−03; MRPL15, −2.1E+00, 1.9E−03; GOLGA2, −2.1E+00, 7.5E−05; EIF3D, |
| | −2.1E+00, 8.6E−03; CDC123, −2.1E+00, 3.1E−02; ZNF738, −2.1E+00, 3.2E−02; BEND3, −2.1E+00, 1.6E−02; RNF168, −2.1E+00, 4.0E−02; POMP, −2.1E+00, |
| | 4.3E−03; PAK1IP1, −2.1E+00, 9.5E−04; DPY30, −2.1E+00, 4.0E−03; CDC37, −2.1E+00, 6.1E−03; CSTB, −2.1E+00, 5.0E−04; VIM, −2.1E+00, 1.3E−03; YTHDF2, |
| | −2.1E+00, 5.0E−02; SMIM7, −2.1E+00, 1.6E−03; C19orf53, −2.1E+00, 1.2E−04; GTF3C4, −2.1E+00, 7.5E−03; CASP3, −2.1E+00, 2.1E−03; TMEM261, |
| | −2.1E+00, 1.4E−03; ATP5G1, −2.1E+00, 9.2E−06; UBL5, −2.1E+00, 7.8E−04; DCTN1, −2.1E+00, 7.9E−03; SNRPG, −2.1E+00, 5.3E−04; DANCR, −2.1E+00, |
| | 1.5E−03; TRIM59, −2.1E+00, 7.2E−03; THY1, −2.1E+00, 4.1E−05; TXNDC17, −2.1E+00, 2.2E−03; MTIF2, −2.1E+00, 6.2E−03; HAUS1, −2.1E+00, 9.1E−03; BOD1, |
| | −2.1E+00, 5.4E−04; EIF2A, −2.1E+00, 2.1E−03; KRT19, −2.1E+00, 1.1E−03; RAB5A, −2.1E+00, 4.1E−02; SFRP2, −2.1E+00, 4.8E−04; RAPGEF5, −2.1E+00, |
| | 1.9E−02; UBQLN2, −2.1E+00, 1.8E−04; EIF2S1, −2.0E+00, 1.8E−03; HIST1H3F, −2.0E+00, 1.3E−03; SARAF, −2.0E+00, 1.4E−02; GTF3C6, −2.0E+00, 3.9E−04; |
| | TMEM14C, −2.0E+00, 2.6E−03; U2AF2, −2.0E+00, 2.6E−03; GDE1, −2.0E+00, 3.2E−04; GPATCH4, −2.0E+00, 3.6E−03; CKAP4, −2.0E+00, 4.6E−04; NIPSNAP1, |
| | −2.0E+00, 2.6E−02; CRKL, −2.0E+00, 1.0E−02; RPL7L1, −2.0E+00, 3.3E−02; NCOA4, −2.0E+00, 7.2E−03; NPC2, −2.0E+00, 1.6E−03; AHSA1, −2.0E+00, |
| | 1.4E−03; MRPL11, −2.0E+00, 2.8E−05; ADSL, −2.0E+00, 8.5E−03; SV2A, −2.0E+00, 6.4E−04; CTSV, −2.0E+00, 2.3E−03; KLHL24, −2.0E+00, 3.4E−03; BEX1, |
| | −2.0E+00, 9.4E−05; ARF6, −2.0E+00, 3.6E−04; CARHSP1, −2.0E+00, 2.7E−02; SERINC1, −2.0E+00, 7.5E−03; MT1X, −2.0E+00, 3.1E−05; NCBP1, −2.0E+00, |
| | 1.1E−02; MESDC2, −2.0E+00, 9.0E−03; ANXA5, −2.0E+00, 2.8E−02; MRPL32, −2.0E+00, 1.0E−03; VPS35, −2.0E+00, 1.9E−02; GGCT, −2.0E+00, 1.0E−02; PPIB, |
| | −2.0E+00, 8.7E−05; P4HB, −2.0E+00, 2.8E−02; GTF2A2, −2.0E+00, 2.4E−03; IARS2, −2.0E+00, 2.7E−02; HIST1H2BH, −2.0E+00, 8.5E−03; COX6C, −2.0E+00, |
| | 8.3E−03; DYNLT1, −2.0E+00, 4.3E−05; TCEAL8, −2.0E+00, 1.9E−04; RAB1A, −2.0E+00, 4.6E−02; RPL22L1, −2.0E+00, 1.0E−02; SF3A1, −2.0E+00, 2.7E−03; |
| | HDGF, −2.0E+00, 1.8E−02; STX3, −2.0E+00, 6.0E−03; HTATSF1, −2.0E+00, 7.1E−03; HMGN5, −2.0E+00, 1.6E−03; ASNSD1, −2.0E+00, 3.7E−04; ARPC1A, |
| | −2.0E+00, 4.0E−03; C7orf73, −2.0E+00, 4.1E−04; DYNC1LI1, −2.0E+00, 3.4E−02; GANAB, −1.9E+00, 2.5E−02; ECHS1, −1.9E+00, 6.8E−04; TBCB, −1.9E+00, |
| | 2.8E−04; RDH11, −1.9E+00, 1.0E−02; EXOSC3, −1.9E+00, 5.4E−04; TUFM, −1.9E+00, 1.7E−03; SSR4, −1.9E+00, 2.7E−04; MRPL47, −1.9E+00, 5.2E−03; |
| | NACC1, −1.9E+00, 1.8E−02; TCEB3, −1.9E+00, 4.0E−03; DNAJA2, −1.9E+00, 3.8E−02; ARF4, −1.9E+00, 2.3E−03; 4.4E+04, −1.9E+00, 1.1E−02; UTP14A, |
| | −1.9E+00, 1.1E−04; NDUFB10, −1.9E+00, 4.5E−03; GTPBP4, −1.9E+00, 4.6E−02; DNAJC19, −1.9E+00, 1.2E−03; PFDN4, −1.9E+00, 2.8E−03; UBXN1, −1.9E+00, |
| | 5.9E−04; ZIC3, −1.9E+00, 1.2E−02; NDUFS6, −1.9E+00, 2.3E−02; BCAS2, −1.9E+00, 2.7E−03; VCPIP1, −1.9E+00, 1.0E−03; GPX8, −1.9E+00, 9.3E−04; ERCC6L, |
| | −1.9E+00, 6.8E−04; TOMM22, −1.9E+00, 3.3E−04; PSMC1, −1.9E+00, 2.3E−03; MMADHC, −1.9E+00, 2.9E−02; AP2S1, −1.9E+00, 7.6E−04; EIF3J, −1.9E+00, |
| | 4.4E−02; CNDP2, −1.9E+00, 1.9E−02; MIDN, −1.9E+00, 1.7E−02; SLC1A5, −1.9E+00, 1.7E−02; S100A13, −1.9E+00, 3.8E−04; PWP1, −1.9E+00, 9.0E−03; |
| | EIF4A3, −1.9E+00, 3.8E−03; STAM, −1.8E+00, 3.7E−02; TRAPPC5, −1.8E+00, 5.0E−05; POLD2, −1.8E+00, 1.6E−03; PHAX, −1.8E+00, 2.8E−02; THOC7, |
| | −1.8E+00, 2.6E−03; NDUFAB1, −1.8E+00, 2.1E−02; DLD, −1.8E+00, 3.4E−02; ZNF92, −1.8E+00, 3.5E−02; RNF11, −1.8E+00, 4.6E−03; RBM8A, −1.8E+00, |
| | 1.7E−02; CLNS1A, −1.8E+00, 3.3E−02; ZNF268, −1.8E+00, 1.9E−03; REX02, −1.8E+00, 9.0E−03; MCCC2, −1.8E+00, 3.3E−02; LOC107987279, −1.8E+00, 8.0E−05; |
| | SRRT, −1.8E+00, 3.4E−02; SF3B5, −1.8E+00, 6.0E−05; TSN, −1.8E+00, 1.4E−02; WDR82, −1.8E+00, 1.4E−02; SERF2, −1.8E+00, 4.0E−02; PRDX4, −1.8E+00, |
| | 1.0E−02; MRPL13, −1.8E+00, 3.2E−02; MRPS15, −1.8E+00, 1.1E−02; ARHGD1A, −1.8E+00, 1.4E−03; CLPX, −1.8E+00, 3.4E−02; RIDA, −1.8E+00, 6.2E−05; |
| | HIST1H3H, −1.8E+00, 3.4E−03; COX2, −1.8E+00, 6.4E−03; FTSJ3, −1.8E+00, 1.1E−02; BRK1, −1.8E+00, 5.3E−03; GPN3, −1.8E+00, 1.5E−03; YWHAH, |
| | −1.8E+00, 3.7E−02; RAB5C, −1.8E+00, 1.5E−03; HIST1H31, −1.8E+00, 2.1E−02; BNIP2, −1.8E+00, 2.3E−02; MTCH1, −1.8E+00, 1.6E−02; LRPAP1, −1.8E+00, |
| | 1.6E−02; NEDD8, −1.8E+00, 2.2E−03; MRPS23, −1.8E+00, 1.2E−03; ZFP36L2, −1.8E+00, 1.2E−02; TRMT112, −1.8E+00, 1.2E−03; LINC01356, −1.8E+00, |
| | 7.3E−03; CDH9, −1.8E+00, 1.1E−02; THUMPD3, −1.8E+00, 1.0E−02; HOOK1, −1.8E+00, 2.1E−02; C14orf2, −1.7E+00, 7.8E−03; EMC4, −1.7E+00, 1.7E−03; |
| | NDUFB7, −1.7E+00, 5.4E−03; NDUFB11, −1.7E+00, 4.9E−03; SURF4, −1.7E+00, 1.7E−02; MRPL21, −1.7E+00, 6.6E−03; UTP14C, −1.7E+00, 1.1E−03; |
| | HIST1H2BD, −1.7E+00, 2.3E−02; STT3A, −1.7E+00, 1.7E−02; LOC101927746, −1.7E+00, 1.6E−02; RAB11A, −1.7E+00, 2.3E−02; SCOC, −1.7E+00, 2.6E−03; |
| | NOP10, −1.7E+00, 5.8E−03; MANF, −1.7E+00, 3.2E−03; DRAP1, −1.7E+00, 3.8E−04; SSR2, −1.7E+00, 1.0E−02; PRKCSH, −1.7E+00, 3.1E−02; SLC7A3, |
| | −1.7E+00, 1.9E−02; FRG1, −1.7E+00, 1.4E−04; KIAA1191, −1.7E+00, 1.5E−02; CFAP97, −1.7E+00, 1.4E−02; CEBPG, −1.7E+00, 5.6E−03; POLR3A, −1.7E+00, |
| | 4.6E−02; CCDC58, −1.7E+00, 5.8E−03; TOMM5, −1.7E+00, 1.1E−02; SHISA5, −1.7E+00, 3.3E−02; HIST1H41, −1.7E+00, 4.4E−03; BNIP3L, −1.7E+00, 6.3E−03; |
| | SDHD, −1.7E+00, 7.1E−03; CDK4, −1.7E+00, 3.4E−03; NDUFA5, −1.7E+00, 3.0E−02; PRPS1, −1.7E+00, 3.7E−03; PSMD13, −1.7E+00, 1.0E−02; POLR2K, |
| | −1.6E+00, 2.4E−04; MRPL35, −1.6E+00, 1.1E−03; USMG5, −1.6E+00, 1.4E−02; UTP6, −1.6E+00, 3.1E−02; MRPL9, −1.6E+00, 5.4E−04; EXOSC9, −1.6E+00, |
| | 3.6E−02; UBE2Q1, −1.6E+00, 2.5E−04; CCNK, −1.6E+00, 4.2E−02; NANOG, −1.6E+00, 5.8E−03; VPS4B, −1.6E+00, 3.8E−02; NUDCD2, −1.6E+00, 5.8E−03; |
| | POLR3C, −1.6E+00, 3.7E−03; NAP1L3, −1.6E+00, 9.9E−03; ESCO2, −1.6E+00, 1.2E−02; HSPA14, −1.6E+00, 2.6E−02; PNPLA8, −1.6E+00, 1.2E−02; C5orf51, |
| | −1.6E+00, 2.0E−02; GTF3A, −1.6E+00, 1.3E−02; MPLKIP, −1.6E+00, 1.5E−04; EXOSC8, −1.6E+00, 3.0E−02; KIAA1143, −1.6E+00, 1.5E−02; ALDH3A2, |
| | −1.6E+00, 4.3E−02; COMMD6, −1.6E+00, 7.8E−03; UTP3, −1.6E+00, 3.2E−03; BANF1, −1.6E+00, 5.4E−03; YKT6, −1.6E+00, 2.8E−03; NDUFB5, −1.6E+00, 4.4E−03; |
| | MRPS18B, −1.6E+00, 1.6E−03; HIGD2A, −1.6E+00, 2.4E−04; PEX19, −1.6E+00, 5.4E−04; SNRPB2, −1.6E+00, 3.4E−02; LINC01090, −1.6E+00, 3.5E−02; |
| | ALDH1B1, −1.6E+00, 2.0E−04; BCL7A, −1.6E+00, 9.5E−04; PPID, −1.6E+00, 1.8E−02; WDR77, −1.6E+00, 3.3E−03; MRPL37, −1.6E+00, 1.2E−02; NDUFS8, |
| | −1.6E+00, 1.7E−02; UBFD1, −1.6E+00, 1.3E−02; PSMD3, −1.6E+00, 9.2E−03; TMEM50A, −1.6E+00, 1.8E−02; ZMYM3, −1.6E+00, 1.5E−02; COPS9, −1.6E+00, |
| | 2.8E−03; MRPS7, −1.6E+00, 1.4E−03; LANCL1, −1.6E+00, 1.3E−02; TSPYL1, −1.6E+00, 4.5E−03; CRIP1, −1.6E+00, 1.6E−04; NDUFB6, −1.6E+00, 1.6E−02; |
| | RPA3, −1.6E+00, 3.8E−03; GNG5, −1.6E+00, 7.7E−03; ACTR6, −1.6E+00, 1.3E−03; HIST4H4, −1.5E+00, 8.8E−04; PBX2, −1.5E+00, 7.3E−03; TMX2, −1.5E+00, |
| | 8.4E−04; SH3BGRL3, −1.5E+00, 1.5E−03; ZBTB33, −1.5E+00, 3.2E−04; NDUFA1, −1.5E+00, 1.0E−03; CENPN, −1.5E+00, 1.0E−02; THYN1, −1.5E+00, 3.0E−03; |
| | NUCB1, −1.5E+00, 5.9E−03; BIRC5, −1.5E+00, 2.5E−02; PBDC1, −1.5E+00, 2.2E−02; NGRN, −1.5E+00, 2.6E−02; MCFD2, −1.5E+00, 3.7E−02; TIMM50, |
| | −1.5E+00, 9.3E−03; PSMG1, −1.5E+00, 4.6E−03; GABPA, −1.5E+00, 2.5E−02; BAG4, −1.5E+00, 3.7E−02; SF3A2, −1.5E+00, 7.0E−04; POLR2I, −1.5E+00, |
| | 1.5E−03; MAGOH, −1.5E+00, 4.4E−03; LBH, −1.5E+00, 1.1E−03; UBE2C, −1.5E+00, 2.0E−02; MRPL20, −1.5E+00, 1.3E−02; LYAR, −1.5E+00, 2.6E−02; HMG20B, |
| | −1.5E+00, 1.6E−03; CTSD, −1.5E+00, 1.7E−02; PPP1R15B, −1.5E+00, 3.1E−02; SEC11C, −1.5E+00, 4.0E−03; TMEM183A, −1.5E+00, 9.4E−03; USP39, |
| | −1.5E+00, 2.8E−02; PPP1R7, −1.5E+00, 1.9E−02; MRPL41, −1.5E+00, 6.8E−03; NRBF2, −1.5E+00, 1.6E−03; ARL3, −1.5E+00, 4.0E−02; FLII, −1.5E+00, |
| | 1.3E−02; BAG5, −1.5E+00, 1.2E−02; DCTPP1, −1.5E+00, 7.2E−04; NDUFC1, −1.5E+00, 1.0E−02; XBP1, −1.5E+00, 3.9E−02; NEK2, −1.5E+00, 1.7E−04; UQCRFS1, |
| | −1.5E+00, 2.0E−02; LLPH, −1.5E+00, 1.4E−03; C12orf57, −1.5E+00, 1.9E−02; QARS, −1.5E+00, 3.1E−02; COPS6, −1.5E+00, 1.3E−02; TMEM258, −1.5E+00, |
| | 2.7E−02; CCDC25, −1.5E+00, 2.3E−02; MPP5, −1.5E+00, 6.0E−03; TRIP10, −1.5E+00, 1.1E−02; ALKBH7, −1.5E+00, 1.4E−03; PGAM1, −1.5E+00, 2.0E−02; |
| | DARS2, −1.5E+00, 3.3E−02; MINOS1, −1.4E+00, 1.2E−02; APRT, −1.4E+00, 2.6E−02; POLR2G, −1.4E+00, 6.4E−03; KIAA0430, −1.4E+00, 1.3E−02; MRPS30, |
| | −1.4E+00, 1.4E−02; BAG6, −1.4E+00, 3.1E−02; SRP68, −1.4E+00, 1.7E−02; CLDN7, −1.4E+00, 1.4E−02; CCDC34, −1.4E+00, 4.6E−02; HIST1H2AB, −1.4E+00, |
| | 8.3E−03; SURF6, −1.4E+00, 3.6E−03; SMIM10L1, −1.4E+00, 1.8E−02; MAGOHB, −1.4E+00, 2.3E−02; EXOSC2, −1.4E+00, 4.1E−02; LOC101927721, −1.4E+00, |
| | 7.6E−03; METTL13, −1.4E+00, 2.0E−02; MT1E, −1.4E+00, 1.2E−05; MLLT11, −1.4E+00, 1.0E−02; TRMT6, −1.4E+00, 9.3E−03; CYB5A, −1.4E+00, 3.1E−02; |
| | PHPT1, −1.4E+00, 1.7E−02; HLA-C, −1.4E+00, 1.4E−02; SLC4A1AP, −1.4E+00, 1.7E−02; NTS, −1.4E+00, 7.6E−05; HLA-A, −1.4E+00, 1.3E−02; TGIF2, |
| | −1.4E+00, 1.2E−02; CYC1, −1.4E+00, 9.0E−03; RWDD1, −1.4E+00, 2.3E−02; PSMC4, −1.4E+00, 3.8E−02; PLIN2, −1.4E+00, 2.0E−02; MBD4, −1.4E+00, 4.4E−02; |
| | MT1H, −1.4E+00, 2.3E−04; LY6E, −1.4E+00, 2.6E−03; BLOC1S6, −1.4E+00, 4.6E−02; OAT, −1.4E+00, 3.9E−02; TIMM8B, −1.4E+00, 4.9E−02; OLFML3, |
| | −1.4E+00, 3.0E−02; EI24, −1.4E+00, 3.2E−02; CTGF, −1.4E+00, 1.6E−02; SYF2, −1.4E+00, 1.7E−02; DNAAF2, −1.4E+00, 1.3E−02; PHF23, −1.4E+00, 7.7E−04; |
| | FAM32A, −1.4E+00, 1.4E−02; KRR1, −1.4E+00, 1.7E−02; TTYH3, −1.3E+00, 3.1E−02; NDUFB1, −1.3E+00, 2.3E−02; TSR2, −1.3E+00, 4.8E−03; TIMM10, |
| | −1.3E+00, 4.2E−03; MTERF3, −1.3E+00, 2.0E−02; RND2, −1.3E+00, 3.9E−02; GAL, −1.3E+00, 1.1E−02; HIST1H2BK, −1.3E+00, 4.9E−02; NDUFA3, −1.3E+00, |
| | 2.1E−02; ETFB, −1.3E+00, 7.2E−03; SLC29A1, −1.3E+00, 2.3E−02; ZNF426, −1.3E+00, 4.5E−02; CDC37L1, −1.3E+00, 3.3E−02; OCIAD2, −1.3E+00, 6.6E−03; |
| | CSNK2B, −1.3E+00, 1.2E−02; RNF26, −1.3E+00, 4.6E−03; FKBP1A, −1.3E+00, 3.8E−02; ATP6AP2, −1.3E+00, 4.7E−02; KLHDC3, −1.3E+00, 3.2E−02; C14orf1, |
| | −1.3E+00, 1.1E−02; KLHL9, −1.3E+00, 3.4E−02; GRPEL1, −1.3E+00, 2.8E−03; COX18, −1.3E+00, 3.7E−03; ASS1, −1.3E+00, 4.0E−02; ZNF28, −1.3E+00, |
| | 1.8E−02; MRPL4, −1.3E+00, 3.4E−03; APEH, −1.3E+00, 1.3E−02; CCDC86, −1.3E+00, 2.3E−04; MKNK2, −1.3E+00, 4.4E−02; SLC39A6, −1.3E+00, 4.0E−02; PPP1R2, |
| | −1.3E+00, 1.3E−02; CD59, −1.3E+00, 3.9E−02; MEAF6, −1.3E+00, 3.4E−02; LAMTOR1, −1.2E+00, 1.0E−02; CHRAC1, −1.2E+00, 6.0E−03; EBP, −1.2E+00, 2.2E−02; |
| | ATRAID, −1.2E+00, 1.8E−02; UQCRQ, −1.2E+00, 3.7E−02; SRF, −1.2E+00, 1.1E−02; GSPT2, −1.2E+00, 1.2E−02; DEGS1, −1.2E+00, 2.9E−02; IFITM2, |
| | −1.2E+00, 5.0E−03; GRPEL2, −1.2E+00, 2.2E−02; PPIL1, −1.2E+00, 1.8E−02; ADI1, −1.2E+00, 3.7E−02; FOPNL, −1.2E+00, 4.0E−02; NDUFA8, −1.2E+00, 3.9E−02; |
| | MRPL55, −1.2E+00, 1.2E−03; MRPS33, −1.2E+00, 4.9E−03; TIMM17B, −1.2E+00, 1.0E−03; ERF, −1.2E+00, 2.0E−02; CHCHD1, −1.2E+00, 3.7E−02; ARL16, |
| | −1.2E+00, 7.8E−04; MRPS17, −1.2E+00, 2.5E−02; ABCF2, −1.2E+00, 4.0E−02; EIF2B1, −1.2E+00, 1.8E−02; FUNDC1, −1.2E+00, 3.9E−03; LOC105373177, |
| | −1.2E+00, 9.4E−03; RPS19BP1, −1.2E+00, 1.3E−02; IGFBP2, −1.2E+00, 4.6E−02; DDX20, −1.2E+00, 2.3E−02; COQ10B, −1.2E+00, 9.2E−03; TIMM10B, |
| | −1.2E+00, 2.6E−02; PSMD10, −1.2E+00, 1.8E−02; HIST2H2BF, −1.2E+00, 7.4E−03; HIST1H4F, −1.2E+00, 5.8E−03; CDKN2AIPNL, −1.2E+00, 4.1E−02; UBAC1, |
| | −1.2E+00, 1.8E−02; SNHG6, −1.2E+00, 9.2E−03; POP7, −1.2E+00, 2.9E−02; NOP16, −1.2E+00, 9.2E−03; VANGL2, −1.2E+00, 3.0E−02; TMED7, −1.2E+00, 5.0E−02; |
| | THAP9-AS1, −1.2E+00, 4.1E−02; SYT4, −1.2E+00, 1.8E−03; C11orf57, −1.2E+00, 3.3E−02; ZNHIT1, −1.2E+00, 4.2E−02; SCHLAP1, −1.1E+00, 9.5E−03; |
| | SS18L2, −1.1E+00, 1.9E−02; C12orf10, −1.1E+00, 2.7E−03; SUPT4H1, −1.1E+00, 1.1E−02; FXYD6, −1.1E+00, 4.1E−03; CPNE7, −1.1E+00, 6.0E−03; SEPHS2, |
| | −1.1E+00, 4.4E−02; C16orf13, −1.1E+00, 2.0E−02; TRAPPC4, −1.1E+00, 1.3E−02; MRPL36, −1.1E+00, 4.2E−03; SIVA1, −1.1E+00, 1.3E−02; ATOX1, −1.1E+00, |
| | 8.3E−03; FOXD3-AS1, −1.1E+00, 1.2E−02; HIST1H3J, −1.1E+00, 3.2E−02; NDUFAF4, −1.1E+00, 4.1E−02; RPL26L1, −1.1E+00, 2.4E−02; SNRNP27, −1.1E+00, |
| | 3.6E−02; USF2, −1.1E+00, 4.6E−02; MRPS22, −1.1E+00, 4.6E−02; NUDT1, −1.1E+00, 4.0E−02; UFSP2, −1.1E+00, 1.4E−02; COA6, −1.1E+00, 1.8E−02; |
| | C6orf120, −1.1E+00, 1.0E−02; PDZD4, −1.1E+00, 2.2E−02; NKAP, −1.1E+00, 3.5E−02; TRIAP1, −1.1E+00, 2.0E−02; PQBP1, −1.1E+00, 2.7E−02; CDT1, |
| | −1.1E+00, 3.3E−02; MRPS12, −1.1E+00, 2.7E−03; RBM42, −1.1E+00, 1.9E−02; MRPL28, −1.1E+00, 4.1E−02; ISYNA1, −1.1E+00, 3.8E−02; HIST1H4K, |
| | −1.1E+00, 3.4E−03; TP53RK, −1.0E+00, 7.1E−03; FYTTD1, −1.0E+00, 3.5E−02; AAMP, −1.0E+00, 3.1E−02; LOC101243545, −1.0E+00, 2.4E−02; DCXR, |
| | −1.0E+00, 1.1E−02; CCDC174, −1.0E+00, 1.9E−02; TUBB2A, −1.0E+00, 2.0E−02; HIST2H2AB, −1.0E+00, 4.4E−02; RGS2, −1.0E+00, 4.6E−02; TMEM223, |
| | −1.0E+00, 3.4E−02; WDR46, −1.0E+00, 3.5E−02; TOB1, −1.0E+00, 9.5E−03; PLEKHJ1, −1.0E+00, 3.3E−02; SNRNP25, −1.0E+00, 4.5E−02; MAGEF1, −1.0E+00, |
| | 1.1E−02; MED8, −1.0E+00, 3.2E−03; PIK3C2B, −1.0E+00, 4.1E−02; INA, −1.0E+00, 4.5E−02; HTATIP2, −9.9E−01, 2.5E−02; TMEM41A, −9.8E−01, 2.5E−02; |
| | POLR3K, −9.8E−01, 1.6E−02; C18orf21, −9.7E−01, 4.7E−02; MRPL40, −9.7E−01, 3.4E−03; FABP7, −9.7E−01, 4.4E−03; BMF, −9.6E−01, 8.4E−03; TRIB1, |
| | −9.6E−01, 2.6E−02; IRF2BP2, −9.5E−01, 4.0E−02; WRB, −9.5E−01, 2.8E−03; ACBD7, −9.5E−01, 2.2E−02; ZNF615, −9.4E−01, 3.6E−02; PNMA1, −9.4E−01, 9.6E−03; |
| | RRAGA, −9.4E−01, 1.4E−02; ABT1, −9.3E−01, 1.5E−03; GDF3, −9.3E−01, 3.2E−03; NOL6, −9.2E−01, 2.1E−02; ATP5D, −9.2E−01, 4.0E−02; RIC8A, −9.2E−01, 9.8E−03; |
| | SLC35B1, −9.1E−01, 4.5E−02; COA3, −9.1E−01, 1.0E−02; MN1, −9.1E−01, 3.9E−02; THAP1, −9.1E−01, 1.9E−02; CCDC127, −9.0E−01, 2.7E−02; ZNF184, |
| | −9.0E−01, 3.4E−02; MLST8, −9.0E−01, 4.0E−03; C1orf174, −9.0E−01, 1.9E−02; GIT1, −8.9E−01, 1.6E−02; ITM2A, −8.8E−01, 3.7E−02; FLVCR1-AS1, −8.8E−01, |
| | 7.9E−03; TAGLN, −8.7E−01, 3.3E−02; PRKACA, −8.6E−01, 3.8E−02; FAM96B, −8.6E−01, 4.8E−02; ING2, −8.6E−01, 3.4E−02; FAM210B, −8.5E−01, 4.0E−03; |
| | ZBTB3, −8.3E−01, 2.3E−02; GUK1, −8.3E−01, 4.2E−02; HIST2H2BE, −8.1E−01, 4.4E−02; RNASEH1-AS1, −8.1E−01, 1.9E−02; KCNK6, −8.1E−01, 6.1E−03; COX14, |
| | −7.9E−01, 1.4E−02; ALDOC, −7.9E−01, 3.9E−02; SYT7, −7.9E−01, 1.9E−02; GEMIN4, −7.9E−01, 3.4E−02; LIMD2, −7.8E−01, 3.1E−02; SMIM12, −7.8E−01, 3.8E−02; |
| | VWCE, −7.7E−01, 2.5E−02; PRELID3B, −7.6E−01, 1.1E−02; KLF15, −7.6E−01, 6.3E−03; MTFP1, −7.6E−01, 5.8E−03; MGAT2, −7.5E−01, 3.3E−02; DUSP23, |
| | −7.5E−01, 1.5E−02; AKIP1, −7.5E−01, 1.2E−02; ZNF559, −7.4E−01, 3.8E−02; HINT3, −7.3E−01, 4.9E−02; BIK, −7.2E−01, 2.0E−02; MPG, −7.1E−01, 4.1E−02; |
| | KLHDC8A, −7.1E−01, 4.5E−02; EXOSC6, −6.9E−01, 1.8E−02; NUDT16L1, −6.9E−01, 2.7E−02; RASGRP2, −6.8E−01, 3.4E−02; C11orf71, −6.6E−01, 4.2E−03; |
| | C1orf122, −6.6E−01, 1.4E−02; KLF1, −6.3E−01, 3.3E−02; CST3, −6.2E−01, 5.0E−02; TSSC4, −6.1E−01, 2.7E−02; AIP, −6.1E−01, 1.8E−02; IL27RA, −6.1E−01, 3.5E−02; |
| | ANXA2P3, −5.9E−01, 2.7E−03; RBP1, −5.8E−01, 3.5E−02; DNAJC4, −5.7E−01, 1.4E−02; FBXL15, −5.6E−01, 1.0E−02; UTF1, −5.3E−01, 4.2E−02; CKMT1A, |
| | −5.2E−01, 2.1E−02; TIMP4, −4.8E−01, 2.5E−02; USP27X, −4.4E−01, 1.6E−02; RGS16, −4.1E−01, 4.0E−02; C1orf74, −3.9E−01, 2.3E−02; TCEAL1, −3.8E−01, 9.3E−03; |
| | ZNHIT2, −3.8E−01, 2.4E−02; EFNA3, −3.6E−01, 3.1E−02; ZNF853, −3.4E−01, 9.5E−03; LOC101929541, −3.0E−01, 2.5E−02; PKD2L1, −2.8E−01, 2.5E−02; |
| | LOC105373003, −2.7E−01, 1.9E−02; LOC101929348, 6.9E−02, 4.7E−02; MIR4263, 6.9E−02, 4.7E−02; LOC105370195, 8.4E−02, 4.5E−02; LOC105369545, |
| | 9.8E−02, 4.3E−02; SNORA2B, 1.0E−01, 2.8E−02; LOC105373406, 1.1E−01, 4.6E−02; LOC105374669, 1.2E−01, 3.5E−02; MS4A7, 1.4E−01, 3.6E−02; |
| | LOC100506274, 1.4E−01, 3.7E−02; RNVU1-3, 1.5E−01, 4.6E−02; LOC105375775, 1.5E−01, 3.9E−02; PATE2, 1.5E−01, 4.3E−02; LOC105371760, 1.5E−01, |
| | 2.2E−02; LOC105377559, 1.6E−01, 3.4E−02; LOC105369616, 1.6E−01, 9.7E−03; LOC101927296, 1.7E−01, 4.1E−02; LOC101929524, 1.7E−01, 4.6E−02; |
| | LOC107984631, 1.9E−01, 4.9E−02; ATP6V1B1, 1.9E−01, 4.6E−02; LOC105378410, 2.0E−01, 2.0E−02; HCG8, 2.0E−01, 7.3E−03; LOC105375210, 2.0E−01, |
| | 1.3E−02; LOC105373645, 2.1E−01, 4.0E−02; CCDC190, 2.3E−01, 4.0E−02; LOC107985235, 2.3E−01, 4.2E−02; SNORD14C, 2.3E−01, 1.8E−02; LOC286437, |
| | 2.4E−01, 4.7E−02; LOC105375167, 2.4E−01, 3.8E−02; LOC105371065, 2.4E−01, 4.6E−02; LOC105376603, 2.5E−01, 2.9E−02; NLRP14, 2.5E−01, 1.9E−02; |
| | LOC105379508, 2.6E−01, 4.9E−03; SMARCA5-AS1, 2.6E−01, 6.6E−03; LOC105379613, 2.6E−01, 4.1E−02; LOC105376485, 2.8E−01, 3.5E−02; BTBD16, |
| | 2.8E−01, 3.0E−02; C5orf66, 2.9E−01, 3.5E−02; LOC107986405, 2.9E−01, 3.7E−02; LOC105371161, 3.0E−01, 3.1E−03; LOC107986970, 3.0E−01, 1.8E−02; |
| | LOC105370198, 3.1E−01, 4.6E−02; LOC105369719, 3.2E−01, 2.7E−02; LOC105373684, 3.5E−01, 1.7E−02; LOC105373037, 3.5E−01, 2.2E−02; |
| | LOC107985897, 3.5E−01, 2.2E−02; LOC105370953, 3.5E−01, 1.9E−03; LOC107986643, 3.6E−01, 1.8E−02; TBC1D22A-AS1, 3.7E−01, 3.8E−02; |
| | LOC105369838, 3.7E−01, 2.3E−02; LOC105373511, 3.8E−01, 1.0E−02; LOC339666, 3.8E−01, 9.1E−03; LOC105373578, 3.8E−01, 1.7E−02; LOC105369896, |
| | 3.9E−01, 2.7E−02; ENPP3, 4.1E−01, 1.2E−02; LOC654342, 4.3E−01, 1.8E−02; LINC01049, 4.3E−01, 1.6E−02; TNXB, 4.7E−01, 3.8E−02; LOC105376123, 4.8E−01, |
| | 1.4E−02; LOC105375153, 4.8E−01, 3.8E−02; LOC105369449, 4.8E−01, 4.8E−03; LOC107985342, 4.8E−01, 2.7E−02; LOC107986885, 4.9E−01, 9.8E−03; |
| | TXNRD2, 4.9E−01, 4.7E−02; LOC107986515, 5.0E−01, 3.0E−02; LOC105375902, 5.0E−01, 2.0E−02; LOC283140, 5.1E−01, 3.5E−02; LOC105379151, 5.2E−01, |
| | 1.2E−02; FOXN3-AS2, 5.3E−01, 3.3E−02; LOC105374520, 5.4E−01, 2.3E−04; LOC105376976, 5.4E−01, 2.3E−02; TEKT5, 5.5E−01, 4.1E−02; |
| | LOC105372037, 5.5E−01, 4.3E−02; LOC105379109, 5.5E−01, 3.2E−02; C1orf158, 5.6E−01, 3.0E−02; LOC101928819, 5.6E−01, 1.8E−02; CMKLR1, 5.6E−01, |
| | 2.2E−02; SULT1C2, 5.7E−01, 2.0E−02; MUC19, 5.9E−01, 1.2E−02; MGLL, 5.9E−01, 2.5E−02; ODF3L2, 6.0E−01, 3.7E−02; LOC105379102, 6.1E−01, 2.9E−02; |
| | TRIM58, 6.1E−01, 4.6E−02; ANGPT2, 6.1E−01, 6.7E−04; LOC107986430, 6.2E−01, 1.0E−02; LOC105376087, 6.2E−01, 3.4E−02; STK31, 6.4E−01, 4.4E−03; |
| | SCARA5, 6.5E−01, 7.3E−04; PWAR1, 6.5E−01, 8.8E−03; LOC105370284, 6.6E−01, 1.6E−02; LOC105373813, 6.7E−01, 2.3E−02; LOC105375752, 6.7E−01, |
| | 4.2E−02; LOC105369403, 6.7E−01, 4.4E−02; DOCK8, 6.8E−01, 1.1E−02; CPED1, 6.8E−01, 3.1E−03; LOC107986113, 6.8E−01, 4.4E−03; ANKRD45, 6.8E−01, |
| | 2.9E−03; MPPE1, 6.9E−01, 2.1E−02; LOC101929551, 7.2E−01, 2.4E−02; LOC105374943, 7.2E−01, 1.6E−02; LOC105374264, 7.2E−01, 3.0E−02; FAM13A- |
| | AS1, 7.2E−01, 4.0E−03; RUNX2, 7.3E−01, 3.3E−02; ADGRD1, 7.3E−01, 2.3E−03; MEPE, 7.3E−01, 4.9E−02; LOC105372044, 7.4E−01, 3.8E−02; FAM86DP, |
| | 7.4E−01, 4.4E−02; ATP6V1C2, 7.5E−01, 6.9E−03; LOC105370283, 7.5E−01, 1.6E−02; PTK2B, 7.6E−01, 4.2E−02; LINC00492, 7.6E−01, 4.5E−02; |
| | LOC102724861, 7.7E−01, 3.7E−02; FAM157A, 7.8E−01, 1.8E−03; LOC105379045, 7.8E−01, 4.0E−02; LOC105377177, 7.9E−01, 8.3E−05; LINC01322, 8.0E−01, |
| | 1.7E−03; LOC107985971, 8.0E−01, 3.8E−02; LOC101927239, 8.0E−01, 2.9E−02; LOC105379110, 8.1E−01, 1.5E−03; LOC339874, 8.1E−01, 4.4E−02; |
| | LOC101928331, 8.1E−01, 2.2E−02; AHRR, 8.2E−01, 4.1E−02; LOC105369166, 8.2E−01, 8.5E−03; GRM7-AS3, 8.2E−01, 4.0E−02; LOC105370500, 8.2E−01, |
| | 4.4E−03; LOC105373455, 8.3E−01, 5.0E−02; ARHGAP25, 8.4E−01, 3.6E−03; WDR11-AS1, 8.4E−01, 2.3E−02; LOC105379080, 8.4E−01, 4.0E−03; |
| | LOC105374344, 8.4E−01, 2.6E−02; LOC105378516, 8.7E−01, 8.3E−04; LINC00871, 8.7E−01, 7.0E−03; LOC101928767, 8.8E−01, 4.0E−02; TRPM6, 8.8E−01, |
| | 3.1E−03; LOC102724637, 8.8E−01, 3.5E−02; CASC20, 8.9E−01, 1.2E−02; CPO, 9.0E−01, 1.5E−02; GCM1, 9.1E−01, 1.9E−02; LOC107983974, 9.1E−01, |
| | 4.5E−03; IGSF9, 9.1E−01, 3.2E−02; LOC107986980, 9.3E−01, 1.2E−02; LOC107986620, 9.3E−01, 2.1E−02; DSCAML1, 9.4E−01, 4.8E−03; LOC105377558, |
| | 9.6E−01, 7.4E−03; LOC105371671, 9.6E−01, 2.3E−02; SLC01C1, 9.7E−01, 4.3E−02; LINC00383, 9.7E−01, 2.5E−03; LOC105370803, 9.9E−01, 1.3E−02; IL15, |
| | 9.9E−01, 1.9E−02; NPNT, 9.9E−01, 2.5E−02; CD48, 9.9E−01, 7.9E−03; TOM1L2, 1.0E+00, 1.6E−02; LOC101928765, 1.0E+00, 4.0E−03; GPR89A, 1.0E+00, |
| | 4.4E−02; CNTN6, 1.0E+00, 2.9E−03; ZRANB2-AS2, 1.0E+00, 5.6E−03; PI15, 1.0E+00, 1.1E−02; LOC101927550, 1.0E+00, 3.8E−02; RNF212, 1.1E+00, 8.3E−03; |
| | LOC101927286, 1.1E+00, 2.1E−02; LOC102724858, 1.1E+00, 3.9E−03; SH3D21, 1.1E+00, 3.7E−02; PPP1R1C, 1.1E+00, 1.6E−02; FLVCR2, 1.1E+00, |
| | 3.4E−02; LOC101929028, 1.1E+00, 1.8E−02; CCDC169-SOHLH2, 1.1E+00, 1.7E−02; LOC107986313, 1.1E+00, 4.3E−02; LOC105374010, 1.1E+00, 7.6E−03; |
| | LOC102724542, 1.1E+00, 2.5E−02; ZNF800, 1.1E+00, 2.4E−02; LOC105375181, 1.1E+00, 1.1E−02; LINC00645, 1.1E+00, 2.1E−03; LM03, 1.2E+00, |
| | 3.2E−02; LOC105371077, 1.2E+00, 1.9E−02; LOC105375817, 1.2E+00, 2.2E−04; C12orf42, 1.2E+00, 2.5E−04; LOC105373187, 1.2E+00, 1.1E−02; |
| | LOC105379005, 1.2E+00, 3.6E−02; FGL1, 1.2E+00, 1.0E−05; LOC105376126, 1.2E+00, 4.0E−04; LOC101929549, 1.2E+00, 3.7E−02; LOC105375721, |
| | 1.2E+00, 3.7E−03; WNT3, 1.2E+00, 2.2E−03; DET1, 1.2E+00, 7.3E−03; LINC00648, 1.2E+00, 8.3E−03; LOC105369863, 1.2E+00, 1.6E−03; ADAMTSL4− |
| | AS1, 1.2E+00, 2.9E−02; PAPPA, 1.2E+00, 4.9E−02; LOC105377261, 1.2E+00, 1.1E−04; EPHA1-AS1, 1.3E+00, 1.3E−02; NLRP12, 1.3E+00, 7.3E−03; |
| | WNT9A, 1.3E+00, 6.6E−04; RNF165, 1.3E+00, 6.7E−03; LOC107985172, 1.3E+00, 2.1E−02; LOC105370982, 1.3E+00, 4.1E−03; A4GALT, 1.3E+00, 3.1E−02; |
| | EML6, 1.3E+00, 1.0E−02; LOC101926941, 1.3E+00, 1.3E−02; LOC105373831, 1.3E+00, 1.6E−03; PQLC1, 1.3E+00, 1.2E−02; MX1, 1.3E+00, 8.2E−03; |
| | PBX4, 1.3E+00, 6.5E−03; LDLRAD4, 1.3E+00, 1.7E−02; METAP1D, 1.3E+00, 2.2E−02; ACER2, 1.3E+00, 4.9E−02; FYCO1, 1.3E+00, 2.9E−02; GRIP2, |
| | 1.3E+00, 1.4E−02; GPR141, 1.3E+00, 4.4E−05; LOC105370921, 1.3E+00, 1.5E−02; TMEM117, 1.3E+00, 3.7E−02; ITGA1, 1.4E+00, 3.1E−02; TEC, 1.4E+00, |
| | 2.2E−02; MCTP2, 1.4E+00, 6.7E−03; LOC105369942, 1.4E+00, 3.4E−03; SYNPO2, 1.4E+00, 3.1E−02; LOC105373691, 1.4E+00, 1.2E−06; LOC107986294, |
| | 1.4E+00, 4.0E−02; LOC105374122, 1.4E+00, 9.8E−03; PIK3AP1, 1.4E+00, 3.3E−02; EPHB2, 1.4E+00, 4.4E−02; KANTR, 1.4E+00, 1.5E−02; CCDC7, 1.4E+00, |
| | 1.8E−03; CNOT6L, 1.4E+00, 1.8E−02; PLSCR4, 1.4E+00, 3.4E−03; GAS6, 1.4E+00, 2.0E−03; LINC00641, 1.4E+00, 7.1E−03; CYB5R4, 1.5E+00, 2.2E−02; |
| | TSNARE1, 1.5E+00, 4.2E−02; UBASH3B, 1.5E+00, 2.1E−02; FAM110B, 1.5E+00, 4.8E−02; LOC107984258, 1.5E+00, 6.2E−03; NBPF11, 1.5E+00, 1.5E−03; |
| | LOC102724880, 1.5E+00, 6.6E−06; LOC105373900, 1.5E+00, 9.0E−03; LOC105375704, 1.5E+00, 2.0E−03; LM07, 1.5E+00, 1.9E−02; ZSCAN5A, 1.5E+00, |
| | 3.9E−02; PRKCH, 1.5E+00, 3.8E−03; LINC01416, 1.5E+00, 3.6E−02; ZYG11A, 1.5E+00, 2.8E−02; EPB41L1, 1.5E+00, 4.9E−02; SLIT3, 1.5E+00, 2.2E−02; |
| | DYNC111, 1.5E+00, 1.6E−02; CHST8, 1.5E+00, 3.7E−02; THUMPD3-AS1, 1.5E+00, 3.5E−02; TBC1D19, 1.6E+00, 2.7E−02; EFR3B, 1.6E+00, 6.2E−03; |
| | LOC105373158, 1.6E+00, 1.6E−04; LOC107984570, 1.6E+00, 6.2E−04; LOC101927768, 1.6E+00, 2.6E−02; LOC105369944, 1.6E+00, 1.9E−03; |
| | MIR99AHG, 1.6E+00, 7.1E−05; NSUN3, 1.6E+00, 4.1E−03; TPTE2P1, 1.6E+00, 5.4E−03; COLEC12, 1.6E+00, 1.2E−02; NPFFR2, 1.6E+00, 1.5E−03; TRIM16, |
| | 1.6E+00, 1.6E−02; LOC107984494, 1.6E+00, 6.4E−04; POLK, 1.6E+00, 1.5E−02; LOC105374656, 1.6E+00, 8.2E−12; LOC107987100, 1.6E+00, 6.8E−03; |
| | APLF, 1.6E+00, 2.9E−02; PDXDC2P, 1.6E+00, 4.5E−02; TVP23C−CDRT4, 1.6E+00, 4.0E−02; USP12, 1.6E+00, 3.4E−02; LOC105369168, 1.6E+00, 7.5E−04; |
| | MTBP, 1.6E+00, 4.2E−02; LOC107987043, 1.6E+00, 1.4E−05; CCDC57, 1.6E+00, 4.5E−02; TESK2, 1.7E+00, 3.5E−02; NOCT, 1.7E+00, 1.4E−02; BANP, |
| | 1.7E+00, 3.4E−02; LINC00491, 1.7E+00, 2.9E−04; C8orf34, 1.7E+00, 6.3E−03; ZNF516, 1.7E+00, 1.0E−02; XRRA1, 1.7E+00, 3.8E−02; LOC107985647, |
| | 1.7E+00, 2.7E−02; GALNT8, 1.7E+00, 2.3E−03; ST7L, 1.7E+00, 3.6E−02; LOC107985325, 1.7E+00, 2.3E−04; PARK2, 1.7E+00, 4.4E−02; PCGF3, 1.7E+00, |
| | 3.4E−02; LOC101929563, 1.7E+00, 6.7E−03; RAB3IP, 1.8E+00, 2.2E−02; PEX14, 1.8E+00, 4.5E−02; DSCAM, 1.8E+00, 4.3E−04; UNC5B, 1.8E+00, 1.7E−02; |
| | DYSF, 1.8E+00, 2.5E−03; ARRB1, 1.8E+00, 1.4E−02; CDHR3, 1.8E+00, 2.0E−02; CHCHD6, 1.8E+00, 4.1E−02; ANO5, 1.8E+00, 2.3E−02; LOC101928565, |
| | 1.8E+00, 2.1E−04; C4orf19, 1.8E+00, 5.3E−04; LOC107985704, 1.8E+00, 8.8E−03; BBOX1-AS1, 1.8E+00, 8.0E−04; NAPB, 1.8E+00, 4.7E−03; SIK2, |
| | 1.8E+00, 1.3E−02; LOC105373456, 1.8E+00, 9.5E−03; LOC102723694, 1.8E+00, 2.0E−03; FBXL4, 1.8E+00, 3.8E−02; TGFBR2, 1.8E+00, 3.2E−03; |
| | LINC01108, 1.9E+00, 7.8E−04; ELF1, 1.9E+00, 4.4E−02; STON2, 1.9E+00, 4.7E−02; RNF217, 1.9E+00, 1.5E−02; LOC105372130, 1.9E+00, 2.1E−03; |
| | LOC107986066, 1.9E+00, 4.9E−04; LONP2, 1.9E+00, 1.4E−02; UQCC1, 1.9E+00, 3.2E−02; LOC105374971, 1.9E+00, 4.0E−03; SLC23A2, 1.9E+00, |
| | 1.8E−02; AGBL1, 1.9E+00, 3.0E−06; LOC349160, 1.9E+00, 2.0E−05; PINX1, 1.9E+00, 3.0E−02; NOVA1, 1.9E+00, 4.9E−02; LOC100507006, 1.9E+00, 1.2E−02; |
| | NNT, 1.9E+00, 3.7E−03; AKAP10, 1.9E+00, 4.6E−02; TAF1B, 1.9E+00, 2.0E−02; RDH13, 1.9E+00, 1.8E−03; MTSS1, 1.9E+00, 1.2E−02; SFSWAP, 1.9E+00, |
| | 4.0E−02; DDHD1, 1.9E+00, 3.6E−02; ACSS3, 2.0E+00, 4.5E−02; TMEM161B, 2.0E+00, 3.1E−02; LOC107987296, 2.0E+00, 5.8E−08; HS3ST3A1, 2.0E+00, |
| | 3.0E−03; LOC105373785, 2.0E+00, 2.0E−03; ZFYVE16, 2.0E+00, 2.0E−02; CASC9, 2.0E+00, 4.9E−03; NUP50-AS1, 2.0E+00, 7.1E−03; KLHDC10, 2.0E+00, |
| | 1.0E−02; LOC105370777, 2.0E+00, 1.2E−03; CMC1, 2.0E+00, 2.1E−02; ARHGAP10, 2.0E+00, 2.1E−03; SPOP, 2.0E+00, 2.6E−03; DENND5A, 2.0E+00, 2.4E−02; |
| | MICU3, 2.0E+00, 5.3E−03; RABGEF1, 2.0E+00, 4.1E−02; TMEM120B, 2.0E+00, 7.5E−03; RIC1, 2.0E+00, 2.1E−02; TEAD4, 2.0E+00, 1.3E−02; |
| | LOC101929147, 2.0E+00, 1.8E−02; LOC105372950, 2.0E+00, 3.6E−07; CHM, 2.0E+00, 2.6E−02; EVC2, 2.0E+00, 2.1E−03; FGGY, 2.0E+00, 2.1E−02; |
| | LINC01456, 2.0E+00, 3.6E−03; C5orf42, 2.1E+00, 2.7E−02; LOC105370504, 2.1E+00, 2.0E−02; USH2A, 2.1E+00, 1.5E−03; KPNA1, 2.1E+00, 3.4E−02; |
| | SERGEF, 2.1E+00, 9.2E−03; DEPTOR, 2.1E+00, 5.3E−03; LOC105370456, 2.1E+00, 5.3E−04; JMJD1C, 2.1E+00, 4.2E−02; ENOSF1, 2.1E+00, 3.9E−02; |
| | SFMBT2, 2.1E+00, 1.9E−02; SPINK5, 2.1E+00, 3.3E−03; DISC1FP1, 2.1E+00, 8.5E−04; GRIA4, 2.1E+00, 4.9E−04; SMAD3, 2.1E+00, 6.8E−03; RAB12, |
| | 2.1E+00, 1.6E−02; TRAK1, 2.1E+00, 3.2E−02; ARMC8, 2.1E+00, 2.5E−02; CEP95, 2.1E+00, 2.9E−02; ZDHHC21, 2.1E+00, 3.2E−02; RYR1, 2.1E+00, |
| | 1.0E−02; CCDC109B, 2.1E+00, 4.4E−03; KC6, 2.1E+00, 3.5E−05; TRAPPC12, 2.1E+00, 1.4E−02; LOC105378030, 2.1E+00, 2.6E−03; NUBPL, 2.1E+00, 3.8E−03; |
| | COX10, 2.1E+00, 8.6E−03; AFF2, 2.1E+00, 2.6E−02; GALK2, 2.1E+00, 1.7E−03; SYNPR, 2.2E+00, 6.3E−04; ZNRF1, 2.2E+00, 2.9E−02; VSNL1, 2.2E+00, |
| | 3.4E−03; LOC105374020, 2.2E+00, 7.9E−05; PDZRN3, 2.2E+00, 3.5E−02; XRCC4, 2.2E+00, 2.1E−02; SLC25A12, 2.2E+00, 2.7E−03; DENND1B, 2.2E+00, |
| | 1.3E−03; EPHA7, 2.2E+00, 7.7E−03; EFCAB13, 2.2E+00, 2.1E−03; MON2, 2.2E+00, 3.5E−02; LOC107984117, 2.2E+00, 1.8E−12; PMEPA1, 2.2E+00, 2.0E−04; |
| | STXBP4, 2.2E+00, 2.8E−02; MFAP3L, 2.2E+00, 3.1E−04; ZNF75D, 2.2E+00, 3.2E−02; GPCPD1, 2.2E+00, 4.2E−03; FTX, 2.2E+00, 4.7E−02; VWDE, |
| | 2.2E+00, 3.5E−02; SENP5, 2.2E+00, 2.2E−02; MAST2, 2.2E+00, 2.7E−02; MIR4435-2HG, 2.2E+00, 7.0E−06; LOC101927605, 2.2E+00, 4.9E−04; |
| | LOC100505498, 2.2E+00, 2.0E−04; MBOAT1, 2.2E+00, 2.6E−05; KCNIP4, 2.2E+00, 8.8E−03; LOC107985165, 2.2E+00, 4.0E−03; ZNF91, 2.3E+00, 3.9E−02; |
| | RASGRF2, 2.3E+00, 2.0E−03; MAGI3, 2.3E+00, 3.6E−02; EVI5, 2.3E+00, 2.0E−02; NSMAF, 2.3E+00, 2.3E−03; LOC105369427, 2.3E+00, 1.3E−09; |
| | KIAA1468, 2.3E+00, 2.5E−02; EEA1, 2.3E+00, 3.3E−02; LINC01508, 2.3E+00, 8.2E−03; HIRA, 2.3E+00, 4.0E−03; ZC3H12C, 2.3E+00, 1.1E−03; IMMP1L, |
| | 2.3E+00, 1.2E−02; LOC101928923, 2.3E+00, 3.1E−03; LOC105377134, 2.3E+00, 2.6E−02; PLCE1, 2.3E+00, 7.6E−07; KLRG2, 2.3E+00, 3.8E−03; |
| | LOC102724623, 2.3E+00, 5.2E−05; SEMA6D, 2.3E+00, 3.9E−04; STX8, 2.3E+00, 1.7E−02; CAMK2D, 2.3E+00, 3.0E−03; ANKDD1B, 2.3E+00, 2.6E−06; |
| | ACVR1, 2.3E+00, 4.0E−03; ALG13, 2.3E+00, 3.0E−02; SAMD12, 2.3E+00, 2.3E−02; HTT, 2.3E+00, 3.1E−02; TLE4, 2.3E+00, 3.1E−02; TSEN2, 2.4E+00, 2.4E−03; |
| | MED27, 2.4E+00, 9.9E−03; PIBF1, 2.4E+00, 2.3E−02; LOC105375161, 2.4E+00, 3.5E−06; MBOAT2, 2.4E+00, 1.8E−02; HOOK3, 2.4E+00, 9.3E−03; |
| | WIPF3, 2.4E+00, 4.1E−04; LOC102724001, 2.4E+00, 9.7E−03; DENND4C, 2.4E+00, 1.7E−02; MPRIP, 2.4E+00, 8.3E−03; DNAJC15, 2.4E+00, 4.2E−05; |
| | CLEC16A, 2.4E+00, 6.4E−03; GRB10, 2.4E+00, 4.3E−02; HHLA1, 2.4E+00, 2.6E−02; RAD54B, 2.4E+00, 1.4E−02; FAM78B, 2.4E+00, 5.5E−06; MAGED1, |
| | 2.4E+00, 4.7E−02; WRN, 2.4E+00, 3.5E−02; ME1, 2.4E+00, 2.4E−04; CCDC150, 2.4E+00, 8.5E−03; LIMK2, 2.4E+00, 2.0E−03; RIMKLB, 2.4E+00, 3.6E−02; |
| | UBE2W, 2.4E+00, 3.0E−02; RCOR3, 2.4E+00, 5.6E−03; KIAA1328, 2.4E+00, 8.6E−03; MAD1L1, 2.4E+00, 1.2E−02; MGMT, 2.4E+00, 5.8E−03; ATXN2, |
| | 2.4E+00, 4.3E−02; NRF1, 2.4E+00, 1.5E−02; PRR16, 2.4E+00, 2.2E−02; XYLT1, 2.4E+00, 2.8E−02; PSD3, 2.4E+00, 4.1E−02; CCSER2, 2.4E+00, 2.1E−02; |
| | RCOR1, 2.5E+00, 4.0E−02; DDX31, 2.5E+00, 8.6E−04; CBLB, 2.5E+00, 2.5E−02; GAB2, 2.5E+00, 1.7E−03; MAML3, 2.5E+00, 1.4E−02; SIPA1L2, 2.5E+00, |
| | 2.3E−03; UBE2E3, 2.5E+00, 2.8E−02; EMSY, 2.5E+00, 1.4E−02; KLF7, 2.5E+00, 9.0E−03; NDUFAF2, 2.5E+00, 3.7E−03; TXLNGY, 2.5E+00, 1.6E−02; MYO19, |
| | 2.5E+00, 1.0E−02; UBE3D, 2.5E+00, 3.6E−02; RAB28, 2.5E+00, 1.8E−02; PDE7A, 2.5E+00, 1.4E−02; ZC3H12B, 2.5E+00, 4.9E−02; LOC105369791, |
| | 2.5E+00, 3.0E−05; CHSY1, 2.5E+00, 7.8E−03; RELL1, 2.5E+00, 1.3E−04; FAM13A, 2.5E+00, 1.9E−02; TBCA, 2.5E+00, 1.0E−02; NEB, 2.5E+00, 1.0E−03; |
| | C11orf49, 2.5E+00, 8.5E−03; CNTLN, 2.5E+00, 4.0E−02; MBNL1, 2.5E+00, 1.1E−02; MAGI1, 2.5E+00, 6.1E−03; C2CD5, 2.5E+00, 1.2E−02; ACOXL, 2.5E+00, |
| | 3.5E−02; TENM4, 2.5E+00, 3.1E−02; HTR2C, 2.5E+00, 5.7E−03; LYPLAL1, 2.5E+00, 8.0E−03; UVRAG, 2.5E+00, 2.0E−02; NEK7, 2.5E+00, 3.9E−03; POT1, |
| | 2.5E+00, 4.0E−04; CDK5RAP2, 2.5E+00, 1.9E−02; STOX2, 2.5E+00, 2.3E−03; PARD3, 2.5E+00, 1.8E−02; SENP7, 2.5E+00, 2.3E−04; LINC01060, 2.5E+00, |
| | 4.4E−03; 4.4E+04, 2.6E+00, 3.8E−03; MYO5A, 2.6E+00, 5.7E−03; TJP2, 2.6E+00, 3.5E−02; PCMTD1, 2.6E+00, 1.9E−02; GATAD2B, 2.6E+00, 1.9E−02; |
| | ATF7IP2, 2.6E+00, 5.6E−03; CHCHD3, 2.6E+00, 3.4E−02; SND1, 2.6E+00, 3.1E−02; VRK2, 2.6E+00, 9.8E−03; USP32, 2.6E+00, 3.5E−02; CHD9, 2.6E+00, |
| | 3.6E−02; CEP192, 2.6E+00, 1.8E−02; TACC2, 2.6E+00, 1.2E−03; LDLRAD3, 2.6E+00, 2.7E−02; WDR33, 2.6E+00, 1.8E−02; PHKB, 2.6E+00, 9.7E−03; |
| | LOC107986724, 2.6E+00, 9.1E−08; RSRC1, 2.6E+00, 2.2E−02; LUZP2, 2.6E+00, 2.5E−02; CLASP2, 2.6E+00, 3.0E−02; RGL1, 2.6E+00, 1.5E−05; ATG5, |
| | 2.6E+00, 6.9E−03; FAM135A, 2.6E+00, 1.6E−02; SCN8A, 2.6E+00, 2.3E−02; CRMP1, 2.6E+00, 1.5E−03; PTAR1, 2.6E+00, 3.3E−02; LINC00630, 2.6E+00, |
| | 4.9E−05; BMPR2, 2.6E+00, 1.9E−02; CHN1, 2.7E+00, 2.2E−02; JARID2, 2.7E+00, 1.4E−03; TRIML2, 2.7E+00, 1.2E−04; LOC105370463, 2.7E+00, 1.7E−08; |
| | KIRREL1, 2.7E+00, 4.2E−03; LOC105373703, 2.7E+00, 3.9E−05; SUZ12P1, 2.7E+00, 2.3E−02; FOXP1, 2.7E+00, 2.2E−02; FRYL, 2.7E+00, 2.2E−02; NARS2, |
| | 2.7E+00, 3.3E−03; KDM6A, 2.7E+00, 2.9E−02; FMNL2, 2.7E+00, 3.3E−02; PDE5A, 2.7E+00, 4.5E−03; SUCLG2, 2.7E+00, 8.5E−03; RPTOR, 2.7E+00, 4.0E−03; |
| | FYN, 2.7E+00, 6.0E−03; CDH8, 2.7E+00, 1.8E−03; PRKCQ, 2.7E+00, 6.9E−04; INVS, 2.7E+00, 6.0E−03; RAPGEF6, 2.7E+00, 7.6E−03; LDAH, 2.7E+00, |
| | 3.5E−03; ATXN7, 2.7E+00, 7.6E−04; LOC107987087, 2.7E+00, 1.8E−02; ASTN2, 2.7E+00, 2.0E−02; XP04, 2.7E+00, 1.2E−02; COL4A2, 2.7E+00, 5.8E−03; |
| | CRIM1, 2.7E+00, 9.9E−03; GLI2, 2.7E+00, 2.2E−02; MYO3A, 2.7E+00, 1.0E−04; WWOX, 2.7E+00, 2.1E−02; CTDSPL, 2.8E+00, 4.0E−03; STK3, 2.8E+00, |
| | 9.0E−03; FGF12, 2.8E+00, 5.1E−03; LOC107985675, 2.8E+00, 1.5E−02; TTLL5, 2.8E+00, 1.3E−02; TRIO, 2.8E+00, 1.9E−02; ARHGAP32, 2.8E+00, 1.6E−02; |
| | FLRT2, 2.8E+00, 1.1E−04; FEZ2, 2.8E+00, 3.1E−04; ZNF609, 2.8E+00, 1.1E−02; CENPC, 2.8E+00, 4.0E−04; RRAS2, 2.8E+00, 1.7E−02; MALAT1, 2.8E+00, |
| | 7.4E−11; ASH1L, 2.8E+00, 1.5E−02; ATG7, 2.8E+00, 3.2E−03; NCOA2, 2.8E+00, 1.5E−02; PKD1L1, 2.8E+00, 2.0E−07; KMT2C, 2.8E+00, 1.8E−02; SLCO1A2, |
| | 2.8E+00, 1.3E−06; RAP1GDS1, 2.8E+00, 3.0E−03; LOC101929095, 2.8E+00, 4.0E−04; UBR3, 2.8E+00, 2.4E−03; AKT3, 2.8E+00, 1.3E−02; LINC01250, |
| | 2.8E+00, 7.7E−14; ERCC8, 2.8E+00, 8.1E−04; ITCH, 2.8E+00, 4.7E−03; MMS22L, 2.8E+00, 1.2E−02; HSD17B12, 2.8E+00, 1.8E−02; LOC105371953, |
| | 2.8E+00, 1.2E−04; LOC100133091, 2.8E+00, 5.0E−03; ANKRD12, 2.8E+00, 7.8E−03; MMP16, 2.8E+00, 9.9E−03; ANKRD11, 2.8E+00, 1.4E−02; HERC4, |
| | 2.8E+00, 9.7E−03; PKP4, 2.8E+00, 1.4E−02; GPATCH2, 2.8E+00, 3.8E−03; RIMS2, 2.9E+00, 9.0E−04; TNRC6A, 2.9E+00, 9.8E−03; GALNT13, 2.9E+00, |
| | 1.5E−02; KCNH8, 2.9E+00, 8.9E−04; MAN1C1, 2.9E+00, 3.8E−06; CAMTA1, 2.9E+00, 1.2E−02; ETNK1, 2.9E+00, 9.3E−03; CCNY, 2.9E+00, 9.5E−03; MICAL3, |
| | 2.9E+00, 1.9E−03; GRIK2, 2.9E+00, 4.5E−05; VPS54, 2.9E+00, 2.2E−03; PDZD2, 2.9E+00, 5.0E−03; GSTCD, 2.9E+00, 2.2E−03; LOC105370108, 2.9E+00, |
| | 5.2E−04; GRIP1, 2.9E+00, 9.6E−03; SRBD1, 2.9E+00, 6.5E−03; AGBL4, 2.9E+00, 3.2E−04; NTN4, 2.9E+00, 1.5E−04; KCNJ3, 2.9E+00, 5.9E−05; UBE3C, |
| | 2.9E+00, 7.4E−03; IL34, 2.9E+00, 3.9E−05; HIBCH, 2.9E+00, 2.8E−03; CELF2, 2.9E+00, 3.9E−03; PCDH11Y, 2.9E+00, 9.0E−05; SLC2A13, 2.9E+00, 2.9E−04; |
| | KCND2, 3.0E+00, 1.1E−02; PCDH15, 3.0E+00, 9.8E−08; FNDC3A, 3.0E+00, 5.9E−03; AGFG1, 3.0E+00, 2.1E−03; CACNA1C, 3.0E+00, 1.9E−05; PARD3B, |
| | 3.0E+00, 6.2E−03; MUC4, 3.0E+00, 6.3E−07; STAG1, 3.0E+00, 1.0E−02; STRN3, 3.0E+00, 6.8E−03; ARHGEF7, 3.0E+00, 2.3E−03; EIPR1, 3.0E+00, 7.5E−04; |
| | RTTN, 3.0E+00, 4.2E−04; MLLT10, 3.0E+00, 8.2E−03; VSIG10, 3.0E+00, 3.5E−03; EHMT1, 3.0E+00, 7.9E−03; RIN2, 3.0E+00, 8.0E−09; KIAA1958, 3.0E+00, |
| | 8.2E−03; CRPPA, 3.0E+00, 2.8E−07; NSMCE2, 3.0E+00, 6.6E−03; DMD, 3.0E+00, 2.3E−02; LOC107986638, 3.0E+00, 1.6E−05; SORBS1, 3.0E+00, 5.0E−03; |
| | CPNE8, 3.0E+00, 1.4E−06; BAZ2B, 3.0E+00, 6.7E−03; RALYL, 3.0E+00, 8.8E−04; PDE3B, 3.0E+00, 6.4E−04; CACNB2, 3.0E+00, 1.1E−03; SLC25A16, |
| | 3.1E+00, 4.3E−05; CEP128, 3.1E+00, 4.2E−03; PLEKHG4B, 3.1E+00, 3.5E−03; UTY, 3.1E+00, 6.7E−03; RUNX1T1, 3.1E+00, 2.0E−03; WDR27, 3.1E+00, |
| | 3.4E−03; TBC1D32, 3.1E+00, 1.7E−03; LOC105378800, 3.1E+00, 5.2E−11; NETO2, 3.1E+00, 2.4E−04; HDAC4, 3.1E+00, 2.5E−03; FOCAD, 3.1E+00, 4.4E−03; |
| | TBC1D23, 3.1E+00, 1.6E−03; TBCK, 3.1E+00, 3.6E−03; KLF12, 3.1E+00, 5.8E−03; DAPK1, 3.1E+00, 5.5E−03; FNDC3B, 3.1E+00, 7.3E−03; DST, 3.1E+00, |
| | 3.9E−04; TTTY14, 3.1E+00, 3.6E−03; SRGAP1, 3.1E+00, 1.8E−04; DIP2C, 3.1E+00, 3.5E−03; LOC105369876, 3.1E+00, 1.7E−06; STX18-AS1, 3.1E+00, 7.5E−04; |
| | ATG10, 3.1E+00, 1.6E−03; KCNQ10T1, 3.2E+00, 2.7E−03; ELP4, 3.2E+00, 2.3E−03; LPP, 3.2E+00, 5.5E−03; MTHFD1L, 3.2E+00, 2.6E−03; |
| | LOC105374657, 3.2E+00, 9.3E−05; SDHAP3, 3.2E+00, 1.2E−04; FBXL7, 3.2E+00, 2.9E−03; CDC42BPA, 3.2E+00, 3.6E−03; FAM160A1, 3.2E+00, 4.1E−03; |
| | TRMT11, 3.2E+00, 7.5E−04; RHBDD1, 3.2E+00, 8.7E−05; CELF4, 3.2E+00, 3.9E−06; XKR6, 3.2E+00, 1.1E−03; BACH2, 3.2E+00, 4.2E−04; VTI1A, 3.2E+00, |
| | 3.6E−03; ASAP1, 3.2E+00, 3.6E−03; RAP1GAP2, 3.2E+00, 5.3E−04; SASH1, 3.2E+00, 8.3E−04; MAP2K4, 3.2E+00, 4.6E−04; MAP2K5, 3.2E+00, 1.4E−04; |
| | DIS3L2, 3.2E+00, 6.5E−05; VASH2, 3.3E+00, 4.4E−03; MIR2052HG, 3.3E+00, 2.0E−08; EXPH5, 3.3E+00, 2.7E−05; ERICH1-AS1, 3.3E+00, 3.7E−05; |
| | CACNA1B, 3.3E+00, 8.8E−05; MBD5, 3.3E+00, 2.6E−03; PBX1, 3.3E+00, 4.5E−06; DOCK4, 3.3E+00, 1.6E−03; TCF7L2, 3.3E+00, 2.8E−05; BTBD9, 3.3E+00, |
| | 4.7E−04; SLX4IP, 3.3E+00, 2.3E−04; BABAM2, 3.3E+00, 1.5E−04; PAN3, 3.3E+00, 1.6E−03; APP, 3.3E+00, 1.2E−03; TNRC6C, 3.3E+00, 5.0E−05; NHS, |
| | 3.3E+00, 3.8E−04; ELMO1, 3.3E+00, 9.4E−05; LOC645513, 3.3E+00, 2.2E−05; KIF16B, 3.3E+00, 8.9E−06; ZRANB3, 3.3E+00, 1.1E−03; PCSK5, 3.4E+00, |
| | 2.5E−04; PPP1R12B, 3.4E+00, 1.6E−05; RALGAPA2, 3.4E+00, 5.6E−04; LCLAT1, 3.4E+00, 2.3E−04; TASP1, 3.4E+00, 6.4E−04; NBAS, 3.4E+00, 4.4E−04; |
| | MIR924HG, 3.4E+00, 4.8E−03; PDSS2, 3.4E+00, 7.2E−04; SPRED1, 3.4E+00, 1.5E−04; ATP9B, 3.4E+00, 4.8E−04; ARHGAP44, 3.4E+00, 5.9E−05; KCNT2, |
| | 3.4E+00, 2.1E−03; ANKS1A, 3.4E+00, 1.9E−04; PKN2, 3.4E+00, 8.3E−04; SUGCT, 3.4E+00, 7.3E−06; GSE1, 3.4E+00, 1.3E−03; FBXW7, 3.4E+00, 8.5E−04; |
| | LOC102723568, 3.4E+00, 3.8E−05; DPP6, 3.5E+00, 1.2E−04; ERBIN, 3.5E+00, 1.4E−03; MB21D2, 3.5E+00, 1.8E−04; UTRN, 3.5E+00, 6.4E−04; DPP10, |
| | 3.5E+00, 1.1E−03; LATS2, 3.5E+00, 3.0E−06; LRBA, 3.5E+00, 3.6E−04; RSU1, 3.5E+00, 4.2E−05; ARID1B, 3.5E+00, 2.2E−04; PATJ, 3.5E+00, 1.1E−03; |
| | RASAL2, 3.5E+00, 9.6E−04; PICALM, 3.5E+00, 2.2E−04; IGF1R, 3.5E+00, 1.5E−04; CECR2, 3.5E+00, 7.1E−05; FARP1, 3.5E+00, 7.9E−04; BCAS3, 3.6E+00, |
| | 5.0E−04; MDGA2, 3.6E+00, 2.1E−06; CTBP2, 3.6E+00, 4.7E−05; FRMPD4, 3.6E+00, 9.9E−07; ATXN7L1, 3.6E+00, 6.4E−04; UST, 3.6E+00, 8.0E−04; |
| | MIR31HG, 3.6E+00, 1.7E−07; PRKG1, 3.6E+00, 8.8E−04; AOPEP, 3.6E+00, 1.6E−04; COG5, 3.6E+00, 3.3E−04; MED13L, 3.6E+00, 5.6E−04; BMPR1A, |
| | 3.6E+00, 6.0E−04; TMEM135, 3.6E+00, 3.1E−04; TLN2, 3.6E+00, 2.4E−04; RFC3, 3.6E+00, 5.6E−04; NHSL1, 3.6E+00, 4.2E−04; CAMKMT, 3.7E+00, 5.8E−04; |
| | NRXN3, 3.7E+00, 7.6E−04; PRIM2, 3.7E+00, 4.2E−04; MEI4, 3.7E+00, 7.8E−08; DNAH14, 3.7E+00, 1.5E−04; SH3RF1, 3.7E+00, 2.6E−05; SIK3, 3.7E+00, |
| | 4.1E−04; ROR2, 3.7E+00, 2.4E−06; DIAPH3, 3.7E+00, 6.5E−04; DOCK1, 3.7E+00, 3.5E−04; SDK1, 3.7E+00, 5.0E−04; ADGRA3, 3.7E+00, 1.9E−04; |
| | LINC00989, 3.7E+00, 6.1E−07; MAPK8, 3.7E+00, 2.3E−04; TAF3, 3.7E+00, 1.6E−05; ST6GALNAC3, 3.8E+00, 3.8E−04; C15orf41, 3.8E+00, 3.3E−05; STK33, |
| | 3.8E+00, 2.3E−04; PVT1, 3.8E+00, 3.0E−04; CCDC141, 3.8E+00, 1.4E−03; MIPOL1, 3.8E+00, 1.1E−04; FASTKD2, 3.8E+00, 2.7E−05; LOC285500, 3.8E+00, |
| | 1.1E−06; BCKDHB, 3.8E+00, 3.3E−04; EHBP1, 3.8E+00, 1.3E−04; AHCYL2, 3.8E+00, 1.6E−04; ITFG1, 3.8E+00, 1.0E−04; LINC01162, 3.9E+00, 3.0E−05; |
| | VAV2, 3.9E+00, 1.1E−05; LOC101927580, 3.9E+00, 1.4E−07; TEX41, 3.9E+00, 1.1E−07; LOC107986978, 3.9E+00, 1.2E−11; LOC107987166, 3.9E+00, |
| | 7.3E−06; PTK2, 3.9E+00, 4.2E−05; EXOC4, 3.9E+00, 6.1E−05; RNF24, 3.9E+00, 1.5E−07; AVL9, 3.9E+00, 1.1E−05; TTC28, 3.9E+00, 1.7E−04; LINC01057, |
| | 3.9E+00, 6.1E−07; TCF4, 3.9E+00, 5.8E−05; FAAH2, 3.9E+00, 5.4E−05; MYO1D, 3.9E+00, 2.9E−05; PITPNC1, 3.9E+00, 1.1E−04; PTPRN2, 3.9E+00, |
| | 1.7E−04; CLASP1, 3.9E+00, 9.9E−05; SPATA5, 3.9E+00, 1.3E−04; NEDD4L, 3.9E+00, 1.6E−04; STPG2, 3.9E+00, 2.9E−07; LOC107986623, 4.0E+00, 5.2E−09; |
| | EDIL3, 4.0E+00, 1.7E−04; ARHGAP42, 4.0E+00, 9.6E−05; FAM155A, 4.0E+00, 1.1E−05; STK39, 4.0E+00, 4.9E−06; TTC17, 4.0E+00, 8.7E−05; RNGTT, |
| | 4.0E+00, 3.0E−05; FAM102B, 4.0E+00, 8.3E−07; MPPED2, 4.0E+00, 1.5E−04; LOC105369617, 4.0E+00, 1.1E−07; IL1RAPL1, 4.0E+00, 2.2E−05; |
| | LOC101928354, 4.0E+00, 1.0E−07; C5orf46, 4.0E+00, 2.6E−06; PDE4D, 4.0E+00, 8.4E−07; ADK, 4.1E+00, 9.9E−05; AGAP1, 4.1E+00, 7.9E−05; |
| | LOC101928283, 4.1E+00, 6.8E−12; TSPAN18, 4.1E+00, 8.2E−07; PCAT14, 4.1E+00, 4.0E−04; PLCB4, 4.1E+00, 3.9E−05; RBPMS, 4.1E+00, 6.9E−07; |
| | POU6F2, 4.1E+00, 1.0E−07; TBC1D22A, 4.1E+00, 2.3E−05; MID1, 4.1E+00, 4.5E−05; ZNF385D, 4.1E+00, 3.2E−08; NTNG1, 4.1E+00, 6.6E−08; TRAPPC9, |
| | 4.1E+00, 5.9E−06; EDA, 4.1E+00, 5.4E−05; KAZN, 4.1E+00, 7.1E−05; COMMD10, 4.2E+00, 6.4E−05; MYO9A, 4.2E+00, 6.0E−06; DOCK3, 4.2E+00, 8.0E−05; |
| | LRFN5, 4.2E+00, 6.2E−05; FTO, 4.2E+00, 2.1E−05; WDR70, 4.2E+00, 5.2E−06; RBMS3, 4.2E+00, 1.1E−06; LOC105375815, 4.2E+00, 8.4E−20; DNAJB4, |
| | 4.2E+00, 1.9E−06; ITPR2, 4.3E+00, 1.9E−05; KLF8, 4.3E+00, 1.3E−05; PLCB1, 4.3E+00, 1.6E−05; TULP4, 4.3E+00, 1.5E−05; CHRM3, 4.3E+00, 3.1E−07; |
| | PTPRK, 4.3E+00, 1.3E−05; LOC105378798, 4.3E+00, 5.4E−08; LOC100288637, 4.3E+00, 2.3E−06; ZFAND3, 4.3E+00, 9.1E−06; FGD4, 4.3E+00, 1.3E−06; |
| | DYM, 4.3E+00, 1.5E−06; SPATA6, 4.3E+00, 1.1E−07; ZNF407, 4.3E+00, 3.2E−07; AFAP1, 4.3E+00, 1.3E−07; PLEKHA5, 4.3E+00, 2.9E−08; SCFD2, 4.3E+00, |
| | 1.8E−07; DANT2, 4.4E+00, 9.9E−06; FARS2, 4.4E+00, 6.6E−06; ADAMTS12, 4.4E+00, 1.8E−06; GLDC, 4.4E+00, 2.3E−07; PTPRG, 4.4E+00, 5.7E−11; CMIP, |
| | 4.4E+00, 9.8E−06; SERINC5, 4.4E+00, 1.5E−05; COP1, 4.4E+00, 5.9E−06; CBFA2T2, 4.4E+00, 1.6E−06; PHKA1, 4.4E+00, 1.9E−11; ACACA, 4.4E+00, 3.4E−06; |
| | SLC16A10, 4.5E+00, 2.8E−06; WDFY3, 4.5E+00, 5.1E−06; LOC107985961, 4.5E+00, 1.0E−06; RORA, 4.5E+00, 1.4E−05; MIR3681HG, 4.5E+00, 3.4E−12; |
| | ADAMTS19, 4.5E+00, 1.1E−05; RBM47, 4.5E+00, 9.2E−06; TMEM132D, 4.5E+00, 9.1E−06; TMCC1, 4.5E+00, 5.0E−06; GABRB3, 4.5E+00, 8.0E−07; |
| | LOC105378797, 4.6E+00, 1.4E−09; NLK, 4.6E+00, 4.4E−07; TMTC1, 4.6E+00, 2.6E−06; MEF2A, 4.6E+00, 1.9E−06; LINC01572, 4.6E+00, 2.9E−06; |
| | TMEM108, 4.6E+00, 5.0E−07; RERE, 4.6E+00, 1.0E−07; DLEU1, 4.6E+00, 2.2E−06; LINC00534, 4.6E+00, 1.9E−11; BICD1, 4.7E+00, 3.7E−09; UGGT2, |
| | 4.7E+00, 1.4E−06; ARHGEF10, 4.7E+00, 1.9E−06; SLC35F3, 4.7E+00, 1.1E−07; ZNRF3, 4.7E+00, 3.1E−07; SUPT3H, 4.7E+00, 1.7E−06; ASCC3, 4.7E+00, |
| | 1.7E−06; GMDS, 4.7E+00, 1.1E−06; TENM3, 4.8E+00, 1.1E−07; RANBP17, 4.8E+00, 1.1E−06; COBL, 4.8E+00, 7.6E−07; TCF12, 4.8E+00, 2.3E−07; SYNE1, |
| | 4.8E+00, 1.3E−10; LOC107985037, 4.8E+00, 2.5E−08; LRCH1, 4.8E+00, 9.0E−11; CHST9, 4.8E+00, 6.8E−07; RFX7, 4.8E+00, 9.4E−07; MACROD2, 4.9E+00, |
| | 3.1E−07; LOC105374945, 4.9E+00, 1.7E−06; CADPS, 4.9E+00, 4.6E−08; LOC107986777, 4.9E+00, 2.5E−06; ANKRD18CP, 4.9E+00, 5.4E−09; TIAM1, |
| | 4.9E+00, 1.4E−07; RPS6KA2, 4.9E+00, 1.8E−07; LOC101928437, 4.9E+00, 2.2E−11; LOC105373153, 5.0E+00, 6.3E−12; PCNX2, 5.0E+00, 4.3E−07; PKIB, |
| | 5.0E+00, 1.3E−06; GRID2, 5.0E+00, 7.9E−35; THADA, 5.1E+00, 4.0E−09; FOXN3, 5.1E+00, 2.4E−18; APBB2, 5.1E+00, 1.3E−07; CTNNA3, 5.1E+00, 8.6E−08; |
| | LINC00922, 5.2E+00, 1.9E−09; UBE2E2, 5.2E+00, 8.6E−09; FER, 5.2E+00, 2.8E−08; LOC105374013, 5.3E+00, 2.0E−10; RYR2, 5.3E+00, 2.3E−07; |
| | LOC101929194, 5.3E+00, 5.5E−08; GRIA1, 5.3E+00, 1.3E−07; PTPRT, 5.3E+00, 3.1E−08; FOXO3, 5.3E+00, 2.2E−09; JAZF1, 5.3E+00, 9.7E−08; EXT1, |
| | 5.4E+00, 9.3E−09; METTL15, 5.4E+00, 1.2E−09; PTCHD1, 5.4E+00, 7.6E−14; TUSC3, 5.4E+00, 6.0E−09; RAD51B, 5.4E+00, 1.2E−08; LOC107985710, |
| | 5.4E+00, 3.2E−08; VPS13B, 5.5E+00, 2.0E−08; LOC105375334, 5.5E+00, 1.1E−10; IMMP2L, 5.5E+00, 1.4E−09; LINC01194, 5.5E+00, 3.7E−09; TBC1D5, |
| | 5.5E+00, 1.3E−08; ULK4, 5.5E+00, 1.5E−09; LOC101929378, 5.5E+00, 2.8E−08; CDH13, 5.6E+00, 1.8E−08; TRPS1, 5.7E+00, 7.9E−10; ST6GAL1, 5.8E+00, |
| | 2.8E−09; LOC105374827, 5.8E+00, 7.2E−18; FBN2, 5.8E+00, 2.6E−11; ANK2, 5.8E+00, 5.4E−23; NEAT1, 5.8E+00, 4.0E−09; GPC6, 5.9E+00, 9.1E−08; |
| | GRIN2A, 6.0E+00, 2.5E−15; FAF1, 6.0E+00, 2.2E−10; TBCD, 6.0E+00, 8.1E−12; MAML2, 6.1E+00, 3.1E−09; SGCD, 6.1E+00, 7.3E−10; LOC105378031, |
| | 6.2E+00, 1.0E−10; ZNF827, 6.2E+00, 1.7E−11; SEMA6A, 6.2E+00, 1.5E−20; ASIC2, 6.3E+00, 1.3E−11; LOC107985962, 6.3E+00, 1.3E−11; CDKAL1, |
| | 6.4E+00, 1.7E−11; SPIDR, 6.4E+00, 6.5E−12; GPHN, 6.5E+00, 9.2E−12; CASC15, 6.5E+00, 8.4E−18; ADCY2, 6.5E+00, 2.2E−09; LOC644919, 6.6E+00, |
| | 1.0E−11; MARCHF1, 6.6E+00, 4.3E−15; VWA8, 6.6E+00, 3.8E−14; OPCML, 6.6E+00, 4.0E−12; FHIT, 6.7E+00, 1.2E−11; SMYD3, 6.7E+00, 8.9E−14; LINGO2, |
| | 6.7E+00, 7.9E−12; C4orf51, 6.7E+00, 1.8E−15; LOC107986770, 6.8E+00, 9.7E−12; LOC100420587, 6.8E+00, 8.5E−13; ARL15, 6.8E+00, 3.6E−13; SYT1, |
| | 6.9E+00, 1.1E−15; BBS9, 6.9E+00, 4.9E−12; SCAPER, 7.2E+00, 3.0E−16; GTDC1, 7.2E+00, 6.0E−17; LOC101927668, 7.2E+00, 1.4E−10; LSAMP, 7.2E+00, |
| | 9.0E−15; NPSR1-AS1, 7.3E+00, 1.2E−17; DPH6, 7.4E+00, 1.8E−16; CACNA2D3, 7.6E+00, 4.1E−16; PPARGC1A, 7.6E+00, 4.4E−16; LAMA2, 7.6E+00, |
| | 1.8E−16; TNS3, 7.7E+00, 2.7E−17; LOC105374655, 7.7E+00, 4.2E−27; KDM4C, 7.9E+00, 3.2E−18; KIAA1217, 7.9E+00, 9.1E−17; TMEFF2, 8.2E+00, 2.1E−23; |
| | LOC339862, 8.5E+00, 1.0E−18; SHROOM3, 8.6E+00, 6.7E−21; LRRTM4, 8.8E+00, 1.5E−33; BHLHE40, 9.0E+00, 5.7E−26; PTCHD1-AS, 9.0E+00, 1.2E−19; |
| | LOC728755, 1.0E+01, 5.2E−25; LOC100505817, 1.1E+01, 6.6E−31; FGF13, 1.1E+01, 3.2E−25; LOC102467213, 1.1E+01, 4.6E−31; CHODL, 1.8E+01, 2.2E−52; |
| 4 | LINC01194, −1.6E+01, 9.8E−12; LINC00428, −1.5E+01, 7.1E−15; ESRG, −1.4E+01, 1.4E−08; SERPINB9, −1.3E+01, 9.7E−10; LOC102467213, −1.2E+01, 2.7E−09; |
| | LOC101927668, −1.0E+01, 1.3E−04; CHODL, −1.0E+01, 4.4E−05; LOC101929194, −9.6E+00, 4.4E−05; LOC105374013, −9.3E+00, 1.9E−09; SERINC5, |
| | −9.1E+00, 7.1E−05; GRID2, −8.7E+00, 1.8E−18; C12orf60, −8.7E+00, 2.1E−08; LOC100505817, −8.3E+00, 4.7E−19; RPLP1, −8.3E+00, 1.2E−03; RACK1, |
| | −8.2E+00, 4.4E−03; PTMA, −7.9E+00, 2.7E−05; ND1, −7.9E+00, 1.3E−04; TERF1, −7.7E+00, 3.0E−03; RPS8, −7.7E+00, 2.4E−03; SERBP1, −7.6E+00, 6.9E−03; |
| | CCDC141, −7.5E+00, 2.1E−09; EIF4A2, −7.3E+00, 1.7E−02; LOC105372310, −7.3E+00, 1.9E−02; SPG20, −7.3E+00, 1.8E−04; USO1, −7.2E+00, 2.5E−02; GJA1, |
| | −7.1E+00, 4.2E−02; RPL5, −7.0E+00, 3.3E−02; NPM1, −7.0E+00, 5.8E−03; BHLHE40, −7.0E+00, 3.0E−08; ZMAT4, −6.9E+00, 1.9E−02; DPPA4, −6.9E+00, 2.8E−02; |
| | LIN28A, −6.9E+00, 1.0E−02; DNMT3B, −6.9E+00, 3.4E−03; MRS2, −6.9E+00, 3.8E−04; PRDX6, −6.8E+00, 1.0E−02; RPL9, −6.8E+00, 3.4E−02; |
| | LOC105374945, −6.7E+00, 1.5E−02; VASH2, −6.6E+00, 4.3E−02; CNBP, −6.5E+00, 2.8E−02; FOXN3, −6.5E+00, 2.3E−03; RPL4, −6.4E+00, 2.6E−02; |
| | HSP90AB1, −6.4E+00, 5.2E−03; C9orf135, −6.4E+00, 4.8E−05; LOC105375710, −6.3E+00, 8.7E−07; FLVCR1, −6.3E+00, 4.9E−02; BUB1, −6.2E+00, 4.8E−02; |
| | CNMD, −6.2E+00, 4.9E−02; LOC105377860, −6.1E+00, 1.8E−02; TDGF1, −6.0E+00, 4.2E−02; LOC107985710, −6.0E+00, 3.6E−06; VSIG10, −5.7E+00, 9.7E−03; |
| | GALNT3, −5.7E+00, 1.4E−02; HSP90AA1, −5.6E+00, 2.6E−02; LRFN5, −5.6E+00, 1.4E−07; LOC105370482, −5.5E+00, 1.6E−02; C5orf46, −5.3E+00, |
| | 1.4E−02; LOC107987166, −4.9E+00, 3.5E−02; SGCD, −4.9E+00, 2.6E−03; HHLA1, −4.9E+00, 2.3E−11; PCAT14, −4.7E+00, 1.2E−02; ATP6, −4.6E+00, 7.1E−03; |
| | LOC107985647, −4.6E+00, 1.0E−07; PLAAT3, −4.6E+00, 1.4E−02; RALYL, −4.5E+00, 4.3E−03; LOC105377901, −4.4E+00, 2.3E−09; GRIA1, −4.3E+00, |
| | 1.3E−02; COX1, −4.3E+00, 1.3E−03; ND5, −4.2E+00, 4.5E−02; LOC105374657, −4.1E+00, 3.9E−03; SPP1, −4.0E+00, 8.6E−04; SNAR−C3, −3.6E+00, 1.2E−02; |
| | C4orf51, −3.6E+00, 1.5E−06; ABHD12B, −3.4E+00, 7.8E−07; FRMPD4, −3.2E+00, 3.4E−02; F3, −3.0E+00, 7.5E−05; LOC107985704, −2.9E+00, 2.9E−03; ND4, |
| | −2.9E+00, 3.9E−02; S100A13, −2.8E+00, 3.8E−02; LOC107987087, −2.8E+00, 1.1E−03; LOC105377986, −2.7E+00, 3.1E−02; C9orf129, −2.6E+00, 1.6E−03; |
| | CCSAP, −2.6E+00, 2.3E−02; LINC00989, −2.3E+00, 7.6E−04; LOC101927915, −2.3E+00, 4.0E−04; MT1X, −2.2E+00, 7.1E−03; LOC107986623, −2.0E+00, |
| | 1.3E−03; LINC01508, −2.0E+00, 1.6E−02; ABCC2, −1.7E+00, 4.8E−02; MT1H, −1.6E+00, 1.2E−03; LOC101927580, −1.4E+00, 4.0E−02; LOC105374814, |
| | −1.2E+00, 1.5E−02; LOC107987090, −9.0E−01, 4.1E−02; LOC105374869, 1.7E−01, 1.3E−02; PMCHL2, 1.7E−01, 1.3E−02; SEMG1, 1.7E−01, 1.3E−02; GCG, |
| | 2.2E−01, 2.2E−03; LOC107985306, 2.2E−01, 2.2E−03; PYHIN1, 2.2E−01, 2.2E−03; SLC17A6, 2.2E−01, 2.2E−03; UCN, 2.2E−01, 2.2E−03; LOC105369406, |
| | 2.2E−01, 1.7E−02; APCS, 2.9E−01, 2.4E−06; LOC105379278, 2.9E−01, 2.4E−06; LOC107984876, 2.9E−01, 2.4E−06; LOC105376645, 3.0E−01, 1.0E−08; |
| | MIR194-2HG, 3.0E−01, 1.0E−08; MIR330, 3.0E−01, 1.0E−08; MIR544B, 3.0E−01, 1.0E−08; FAM228A, 3.0E−01, 2.0E−03; TMEM105, 3.0E−01, 2.0E−03; |
| | SSTR5, 3.0E−01, 2.1E−02; PRRT1, 3.1E−01, 2.8E−02; LINC00508, 3.2E−01, 5.8E−03; ZNF735, 3.2E−01, 5.8E−03; LOC107984008, 3.2E−01, 3.5E−03; ENPP7, |
| | 3.2E−01, 2.4E−02; LOC105372985, 3.4E−01, 1.4E−07; LOC105375532, 3.4E−01, 1.4E−07; LOC105376556, 3.4E−01, 1.4E−07; USP41, 3.4E−01, 1.4E−07; |
| | LOC101927755, 3.5E−01, 3.4E−02; TPPP3, 3.6E−01, 1.4E−04; MYL4, 4.0E−01, 8.6E−04; LOC105372750, 4.3E−01, 2.3E−02; FGFBP1, 4.3E−01, 4.9E−02; |
| | LOC105374769, 4.4E−01, 1.2E−02; HOXB6, 4.6E−01, 7.6E−04; EPHB3, 4.8E−01, 1.0E−02; CABCOCO1, 4.9E−01, 1.6E−02; SLC52A1, 5.5E−01, 4.3E−04; |
| | LOC105379301, 5.8E−01, 5.0E−03; LOC107984512, 6.0E−01, 1.8E−05; SQOR, 6.1E−01, 5.1E−04; SLC26A4, 6.2E−01, 1.4E−03; LOC105369482, 6.8E−01, |
| | 5.6E−04; LOC105372782, 7.0E−01, 1.7E−03; LOC105374496, 7.1E−01, 6.2E−11; HOXD4, 7.3E−01, 1.6E−09; LOC105372821, 7.3E−01, 1.6E−09; |
| | LOC105376027, 7.3E−01, 1.6E−09; RTCA-AS1, 7.3E−01, 4.0E−02; HDC, 7.4E−01, 3.5E−04; DIRAS3, 7.4E−01, 9.5E−07; CCDC9B, 7.4E−01, 1.6E−03; |
| | LOC105376425, 7.5E−01, 5.0E−03; LOC101927244, 7.6E−01, 6.2E−04; LOC100996294, 7.7E−01, 4.7E−04; LOC105374798, 7.9E−01, 3.9E−05; |
| | LOC107985685, 8.0E−01, 5.9E−06; TRIM55, 8.2E−01, 1.8E−02; LIFR-AS1, 8.3E−01, 3.7E−02; TMEFF1, 8.4E−01, 1.4E−02; HOXD3, 8.4E−01, 2.2E−05; ZBED5- |
| | AS1, 8.6E−01, 9.3E−03; SERPINF2, 9.1E−01, 1.9E−02; LOC105369309, 9.3E−01, 9.7E−03; FGF10-AS1, 9.4E−01, 2.8E−10; LOC105376981, 9.4E−01, 2.3E−03; |
| | LOC105373693, 9.6E−01, 3.5E−04; LOC285889, 9.8E−01, 3.8E−26; NTRK2, 9.8E−01, 5.9E−03; KRT222, 9.9E−01, 1.3E−02; OPRM1, 9.9E−01, 4.6E−08; |
| | LOC105369689, 1.0E+00, 7.8E−04; CLEC4D, 1.0E+00, 1.7E−13; MOSPD3, 1.0E+00, 7.7E−03; LOC105375728, 1.0E+00, 3.4E−04; LOC101928529, |
| | 1.0E+00, 1.3E−02; OPN5, 1.0E+00, 5.6E−27; LOC105376440, 1.0E+00, 1.5E−12; KCNS1, 1.0E+00, 7.8E−07; LOC102723724, 1.0E+00, 2.8E−03; |
| | LOC102724392, 1.0E+00, 4.3E−11; LOC102725254, 1.0E+00, 3.3E−10; METTL11B, 1.0E+00, 1.0E−07; LOC107987057, 1.0E+00, 2.7E−02; |
| | LOC105378512, 1.1E+00, 6.0E−04; KRT15, 1.1E+00, 2.3E−11; LOC105377135, 1.1E+00, 2.5E−66; MTRNR2L4, 1.1E+00, 2.5E−66; ARX, 1.1E+00, 5.6E−28; |
| | RAB9B, 1.1E+00, 2.9E−05; LINC01518, 1.1E+00, 3.0E−23; CA5A, 1.1E+00, 3.5E−04; LOC105373137, 1.1E+00, 6.1E−08; LINC00882, 1.1E+00, 3.8E−05; |
| | LOC102724362, 1.1E+00, 3.9E−02; PCDH19, 1.1E+00, 3.5E−02; FGF13-AS1, 1.1E+00, 8.0E−03; SNORD12B, 1.1E+00, 1.0E−13; LOC107985714, 1.1E+00, |
| | 5.0E−20; MIR4256, 1.1E+00, 3.2E−16; PRSS55, 1.1E+00, 8.1E−21; AQP4, 1.1E+00, 2.0E−09; RSPH1, 1.1E+00, 1.2E−04; FBXL22, 1.1E+00, 8.7E−03; |
| | SCGB2B2, 1.1E+00, 2.7E−13; ZNF763, 1.1E+00, 1.2E−02; SNAI3, 1.1E+00, 4.8E−06; LOC100505530, 1.1E+00, 6.2E−03; LOC105377585, 1.1E+00, |
| | 1.0E−02; LOC105379064, 1.1E+00, 3.9E−05; SCX, 1.1E+00, 6.2E−07; ADH6, 1.1E+00, 2.8E−03; MFSD2B, 1.2E+00, 3.0E−03; LOC101927378, 1.2E+00, 1.9E−13; |
| | LOC105378623, 1.2E+00, 2.1E−30; LOC105373292, 1.2E+00, 1.4E−05; LOC107987055, 1.2E+00, 9.1E−33; LOC105379050, 1.2E+00, 1.5E−07; |
| | TMEM115, 1.2E+00, 2.0E−02; LOC101928535, 1.2E+00, 6.3E−31; LOC107986417, 1.2E+00, 3.9E−19; MIR320E, 1.2E+00, 9.7E−26; TCL6, 1.2E+00, |
| | 1.1E−08; ASCL2, 1.2E+00, 5.5E−23; LOC105379365, 1.2E+00, 3.1E−33; PLPPR4, 1.2E+00, 3.8E−03; SYTL3, 1.2E+00, 4.6E−02; EMX2OS, 1.2E+00, 8.1E−21; |
| | EFHB, 1.2E+00, 1.7E−03; IGLL5, 1.2E+00, 7.0E−52; MB, 1.2E+00, 2.5E−15; LSAMP-AS1, 1.2E+00, 1.8E−05; SNORD116−26, 1.2E+00, 1.0E−02; ANKFN1, |
| | 1.2E+00, 4.6E−03; LOC107984315, 1.3E+00, 3.6E−04; LOC105375457, 1.3E+00, 4.4E−06; SLC17A1, 1.3E+00, 2.5E−22; LOC105375803, 1.3E+00, |
| | 1.8E−38; LOC729558, 1.3E+00, 2.8E−03; RGS14, 1.3E+00, 5.6E−04; CDH24, 1.3E+00, 2.7E−02; TINAG, 1.3E+00, 4.7E−34; LOC105376234, 1.4E+00, 3.2E−16; |
| | MRGPRF, 1.4E+00, 1.1E−03; LOC105369927, 1.4E+00, 1.5E−02; PARP12, 1.4E+00, 1.2E−02; SYBU, 1.4E+00, 3.2E−02; FRMPD2, 1.4E+00, 7.6E−04; |
| | LOC105377623, 1.4E+00, 1.0E−02; SP8, 1.4E+00, 2.5E−04; VENTX, 1.4E+00, 7.3E−04; LOC105374165, 1.4E+00, 1.3E−02; LOC105378866, 1.4E+00, |
| | 1.2E−10; LOC105372606, 1.4E+00, 4.7E−19; LOC101927421, 1.5E+00, 8.5E−03; APOL2, 1.5E+00, 4.5E−02; ANAPC15, 1.5E+00, 4.7E−05; EPS8L1, 1.5E+00, |
| | 4.7E−03; LINC02208, 1.5E+00, 7.5E−07; EFCAB12, 1.5E+00, 2.8E−08; IRX5, 1.6E+00, 3.6E−13; LOC105378248, 1.6E+00, 5.2E−03; RHEBL1, 1.6E+00, |
| | 6.9E−08; DYNLT3, 1.6E+00, 5.6E−03; GSTM2, 1.6E+00, 2.1E−03; RELN, 1.6E+00, 5.4E−06; LOC107985770, 1.6E+00, 9.3E−18; LOC105378120, 1.7E+00, |
| | 5.1E−04; LOC101927995, 1.7E+00, 4.9E−31; CDK15, 1.7E+00, 4.1E−02; SLC9B2, 1.7E+00, 3.3E−03; DAB2, 1.7E+00, 1.5E−02; MOXD1, 1.7E+00, 4.1E−02; |
| | MTTP, 1.7E+00, 6.9E−06; ABCA12, 1.8E+00, 1.4E−02; DOCK8, 1.9E+00, 3.6E−02; CDH10, 1.9E+00, 1.7E−02; H2BU1, 1.9E+00, 1.1E−05; TMUB2, 1.9E+00, |
| | 3.2E−02; LOC105375721, 1.9E+00, 3.2E−02; FGF11, 1.9E+00, 2.2E−02; TFAP2A, 1.9E+00, 1.2E−03; ANXA3, 2.0E+00, 5.2E−03; PAX7, 2.1E+00, 2.4E−06; |
| | ADAM12, 2.1E+00, 5.2E−03; GOLGA8B, 2.1E+00, 4.9E−02; MGAM, 2.1E+00, 5.0E−10; LOC105379109, 2.1E+00, 1.4E−10; LOC105378308, 2.2E+00, |
| | 1.5E−02; MAOB, 2.2E+00, 4.8E−43; GLIS3, 2.4E+00, 2.3E−02; RPH3AL, 2.4E+00, 2.8E−02; ANKRD16, 2.4E+00, 1.0E−02; ARID5B, 2.4E+00, 6.8E−04; ME3, |
| | 2.5E+00, 9.6E−03; DPYD, 2.5E+00, 2.3E−04; LOC105373627, 2.5E+00, 1.9E−44; SEMA3C, 2.6E+00, 1.7E−02; FOXP2, 2.6E+00, 8.4E−03; CYP27A1, |
| | 2.6E+00, 3.0E−09; GOLGA8A, 2.7E+00, 8.6E−04; LOC100130207, 2.8E+00, 4.9E−02; PCDH17, 2.9E+00, 4.3E−04; SOX6, 2.9E+00, 4.4E−04; ANGPT1, |
| | 3.0E+00, 3.8E−02; ZNF256, 3.0E+00, 1.1E−04; LOC107987163, 3.1E+00, 2.7E−02; UBE2S, 3.1E+00, 3.5E−04; MALAT1, 3.2E+00, 4.3E−02; BMPR1B, |
| | 3.3E+00, 1.7E−02; LOC105370802, 3.3E+00, 2.8E−02; CA10, 3.4E+00, 4.2E−02; SLC66A2, 3.5E+00, 3.9E−05; NOX4, 3.5E+00, 3.1E−05; GNG12, 3.5E+00, |
| | 1.3E−02; SYTL5, 3.7E+00, 6.2E−08; HMCN1, 3.7E+00, 1.9E−02; POC1B, 3.8E+00, 2.6E−02; PDCD4, 3.9E+00, 1.6E−02; INPP4B, 3.9E+00, 9.1E−05; ZFHX4, |
| | 4.0E+00, 6.4E−04; LPAR1, 4.0E+00, 5.9E−03; MEIS2, 4.1E+00, 8.2E−04; UNC5C, 4.2E+00, 5.6E−03; NCAM1, 4.2E+00, 9.7E−03; KDM3A, 4.3E+00, 2.0E−02; |
| | NCKAP5, 4.3E+00, 7.7E−03; PIGU, 4.3E+00, 5.6E−03; TAFA2, 4.3E+00, 1.2E−03; RSRP1, 4.5E+00, 3.2E−02; DENND1B, 4.6E+00, 2.8E−03; ASXL3, 4.7E+00, |
| | 2.8E−08; LHFP, 4.7E+00, 5.0E−02; ITGB8, 4.7E+00, 6.2E−03; HAPLN1, 4.9E+00, 1.1E−03; LOC105378531, 4.9E+00, 8.1E−04; GRIK2, 5.0E+00, 1.3E−02; |
| | ANKRD6, 5.1E+00, 1.2E−02; ANAPC10, 5.2E+00, 3.2E−02; PPFIBP1, 5.3E+00, 2.0E−02; SDC2, 5.4E+00, 3.9E−02; SH3GL2, 5.4E+00, 4.0E−03; PKNOX2, |
| | 5.4E+00, 3.2E−04; LAMB1, 5.5E+00, 4.4E−02; LOC107986022, 5.5E+00, 1.2E−04; RCAN2, 5.5E+00, 3.8E−05; KCNIP4, 5.8E+00, 1.3E−02; SLC35F1, |
| | 5.8E+00, 4.5E−02; FAM171A1, 5.8E+00, 6.4E−03; TTC3, 5.9E+00, 2.3E−02; CAMK2D, 5.9E+00, 1.0E−02; VAV3, 5.9E+00, 5.8E−03; AUTS2, 6.0E+00, 4.9E−02; |
| | PGAP1, 6.0E+00, 5.0E−02; HIPK2, 6.0E+00, 3.4E−02; CNKSR3, 6.0E+00, 6.4E−04; AHNAK, 6.3E+00, 8.6E−04; ITGAV, 6.5E+00, 3.5E−04; DACH1, |
| | 6.6E+00, 8.4E−06; PKP2, 6.7E+00, 1.5E−07; CHD7, 6.7E+00, 4.7E−02; CASK, 6.8E+00, 3.2E−02; TLE4, 6.9E+00, 1.6E−02; KIF26B, 6.9E+00, 4.4E−09; |
| | ZSWIM6, 6.9E+00, 1.2E−02; MAPK10, 7.0E+00, 5.4E−03; NF1, 7.0E+00, 2.4E−02; LPP, 7.0E+00, 4.1E−02; ARL15, 7.1E+00, 3.3E−02; NLGN4Y, 7.1E+00, |
| | 4.2E−02; SIPA1L2, 7.1E+00, 6.8E−06; CDH2, 7.1E+00, 4.3E−04; VTI1A, 7.2E+00, 2.5E−02; LOC101928570, 7.2E+00, 7.2E−04; COL4A5, 7.2E+00, 1.1E−02; |
| | ZNF521, 7.3E+00, 3.9E−03; PPP3CA, 7.3E+00, 1.2E−02; MAGI3, 7.3E+00, 4.2E−03; NRXN3, 7.3E+00, 3.3E−02; DSP, 7.3E+00, 7.1E−03; PDE4D, 7.3E+00, |
| | 2.8E−03; MAML3, 7.3E+00, 2.0E−03; ZNF608, 7.4E+00, 1.9E−02; SDK1, 7.4E+00, 2.3E−02; PTPRD, 7.6E+00, 1.9E−02; CDH6, 8.2E+00, 3.4E−06; PARP8, |
| | 8.2E+00, 7.6E−04; SLIT2, 8.2E+00, 8.7E−05; PALLD, 8.3E+00, 6.1E−05; FRYL, 8.4E+00, 1.2E−03; TENM4, 8.5E+00, 2.8E−03; PDE10A, 8.6E+00, 5.4E−08; |
| | GPC3, 8.7E+00, 3.9E−03; LRP1B, 8.7E+00, 7.8E−07; EFNA5, 8.7E+00, 2.3E−03; ZBTB20, 8.8E+00, 5.1E−05; CADM1, 8.9E+00, 1.3E−04; SORBS2, 9.0E+00, |
| | 2.5E−06; CTNNA2, 9.5E+00, 5.6E−04; ERBB4, 9.6E+00, 6.7E−07; FBN2, 9.6E+00, 1.2E−06; CNTN4, 9.8E+00, 2.2E−05; PTPRM, 1.0E+01, 1.8E−08; PRTG, |
| | 1.1E+01, 2.7E−13; CACNA2D1, 1.1E+01, 1.6E−05; CDK14, 1.1E+01, 8.6E−08; GREB1L, 1.6E+01, 1.2E−16; |
| 5 | CDH1, −4.1E+00, 2.8E−19; PODXL, −4.1E+00, 1.3E−25; CD24, −3.6E+00, 4.6E−21; DPPA4, −3.3E+00, 1.8E−14; ESRG, −3.2E+00, 1.1E−11; L1TD1, −2.9E+00, |
| | 9.0E−15; GPC4, −2.8E+00, 1.4E−08; GSTP1, −2.8E+00, 5.0E−09; HMGA1, −2.7E+00, 1.6E−08; CHODL, −2.7E+00, 3.6E−05; APELA, −2.7E+00, 3.6E−08; CGNL1, |
| | −2.6E+00, 5.4E−09; SAT1, −2.6E+00, 2.0E−08; LINC01194, −2.6E+00, 1.7E−07; GRID2, −2.6E+00, 4.6E−18; SLIRP, −2.6E+00, 2.9E−07; SLC16A1, −2.6E+00, |
| | 5.7E−08; SERPINB9, −2.5E+00, 4.8E−10; POU5F1, −2.5E+00, 7.6E−07; ITGA6, −2.5E+00, 3.7E−07; OPCML, −2.4E+00, 4.1E−06; ANKRD18CP, −2.4E+00, 2.4E−06; |
| | SLC7A8, −2.4E+00, 1.0E−08; SOX2, −2.4E+00, 1.6E−06; SET, −2.3E+00, 8.9E−08; TDGF1, −2.3E+00, 3.4E−07; UCHL1, −2.3E+00, 6.7E−06; RTN3, −2.3E+00, |
| | 1.8E−06; ATP5F1B, −2.3E+00, 5.2E−06; RPL24, −2.2E+00, 1.5E−05; SEMA3E, −2.2E+00, 1.2E−05; DHCR24, −2.2E+00, 2.3E−05; PTMA, −2.2E+00, 9.0E−14; |
| | RPL35, −2.2E+00, 7.1E−05; DNMT3B, −2.1E+00, 1.3E−06; CTSC, −2.1E+00, 3.1E−05; PLS3, −2.0E+00, 8.5E−05; CLDN6, −2.0E+00, 1.2E−05; CLU, −2.0E+00, |
| | 2.1E−05; EPCAM, −2.0E+00, 3.9E−05; LOC101927668, −2.0E+00, 2.1E−05; AKIRIN1, −2.0E+00, 1.1E−04; H4C3, −2.0E+00, 4.2E−03; GAPDH, −2.0E+00, 9.1E−08; |
| | CTSV, −2.0E+00, 7.7E−06; BEX3, −1.9E+00, 6.1E−04; RPS27, −1.9E+00, 3.2E−04; ESRP1, −1.9E+00, 7.5E−05; CALM1, −1.9E+00, 3.7E−04; HMGB3, |
| | −1.9E+00, 3.0E−04; GJA1, −1.9E+00, 1.6E−04; EZR, −1.9E+00, 2.4E−04; HSD17B4, −1.9E+00, 1.7E−04; BNC2, −1.9E+00, 5.1E−04; LARP7, −1.9E+00, 5.7E−04; |
| | RPS14, −1.9E+00, 1.4E−04; LIN28A, −1.9E+00, 7.1E−07; RPL34, −1.9E+00, 1.1E−04; LINC01356, −1.9E+00, 9.0E−09; ATP5IF1, −1.9E+00, 7.3E−05; TENM4, |
| | −1.9E+00, 4.0E−04; TARS1, −1.9E+00, 5.4E−04; RAB3B, −1.8E+00, 2.6E−04; TERF1, −1.8E+00, 1.9E−04; CEBPZ, −1.8E+00, 8.8E−04; RPL12, −1.8E+00, 3.6E−04; |
| | DPYSL3, −1.8E+00, 7.9E−04; UQCRH, −1.8E+00, 3.2E−04; JARID2, −1.8E+00, 8.5E−05; RPL32, −1.8E+00, 1.2E−03; LRRTM4, −1.8E+00, 2.4E−03; RPL21, |
| | −1.8E+00, 1.2E−03; RPL35A, −1.7E+00, 1.5E−03; FOXP1, −1.7E+00, 2.5E−03; SEPHS1, −1.7E+00, 8.7E−04; NTM, −1.7E+00, 4.2E−03; TFRC, −1.7E+00, 3.1E−04; |
| | OSBPL10, −1.7E+00, 2.2E−03; RPS29, −1.7E+00, 3.7E−03; RPL8, −1.7E+00, 1.0E−03; CACHD1, −1.7E+00, 2.9E−03; BPTF, −1.7E+00, 8.9E−04; RPS6, |
| | −1.7E+00, 1.8E−07; PCDH11X, −1.7E+00, 4.0E−03; RPL11, −1.7E+00, 2.2E−04; FTH1, −1.7E+00, 2.4E−03; CXADR, −1.7E+00, 2.8E−03; RPL37, −1.7E+00, 6.0E−03; |
| | SPP1, −1.6E+00, 2.9E−03; BST2, −1.6E+00, 5.5E−06; MAD2L2, −1.6E+00, 4.0E−05; NCL, −1.6E+00, 1.1E−05; RPS21, −1.6E+00, 4.4E−03; ENO1, −1.6E+00, |
| | 2.8E−03; NRXN1, −1.6E+00, 5.8E−03; DBN1, −1.6E+00, 1.7E−03; CLIC4, −1.6E+00, 4.6E−03; PTPRZ1, −1.6E+00, 5.9E−03; RPL31, −1.6E+00, 7.6E−04; RIMS2, |
| | −1.6E+00, 5.1E−03; RCN2, −1.6E+00, 2.1E−03; YWHAE, −1.6E+00, 2.5E−03; LITAF, −1.6E+00, 3.2E−03; HNRNPU, −1.6E+00, 7.6E−04; HSPD1, −1.6E+00, |
| | 8.0E−04; ACOXL, −1.6E+00, 2.1E−03; ZNF281, −1.5E+00, 8.0E−03; UGP2, −1.5E+00, 3.4E−03; FGF13, −1.5E+00, 4.5E−02; SALL4, −1.5E+00, 9.1E−03; PSIP1, |
| | −1.5E+00, 9.5E−03; BTF3, −1.5E+00, 1.9E−02; RPS26, −1.5E+00, 6.9E−03; RPL27, −1.5E+00, 1.4E−02; TFDP2, −1.5E+00, 1.1E−02; MGST1, −1.5E+00, 6.4E−03; |
| | RPS15A, −1.5E+00, 1.0E−02; NEDD4L, −1.5E+00, 1.1E−02; FGFR2, −1.5E+00, 1.4E−02; BICD1, −1.5E+00, 2.1E−02; BCOR, −1.5E+00, 4.3E−03; CFAP298, |
| | −1.5E+00, 1.6E−03; RPL19, −1.4E+00, 5.6E−03; SNTG2, −1.4E+00, 2.9E−02; LDHB, −1.4E+00, 8.3E−03; DBI, −1.4E+00, 2.3E−02; C13orf42, −1.4E+00, |
| | 1.3E−02; MAP7, −1.4E+00, 2.0E−02; PDPN, −1.4E+00, 2.2E−03; EIF3K, −1.4E+00, 1.2E−03; AP2M1, −1.4E+00, 1.9E−02; PRR16, −1.4E+00, 6.6E−03; RPS19, |
| | −1.4E+00, 1.5E−02; PMAIP1, −1.4E+00, 3.5E−03; PARK7, −1.4E+00, 2.6E−02; QSER1, −1.4E+00, 2.7E−02; UQCR10, −1.4E+00, 7.1E−03; RPL23, −1.4E+00, |
| | 1.5E−02; ATP5PD, −1.4E+00, 4.6E−03; SYN3, −1.4E+00, 3.5E−02; EIF5B, −1.4E+00, 3.3E−02; PHF21B, −1.4E+00, 2.9E−02; RPS15, −1.4E+00, 3.7E−02; |
| | ALDOA, −1.4E+00, 2.5E−02; TMED2, −1.4E+00, 2.7E−02; ZNF483, −1.4E+00, 3.6E−03; PSMA2, −1.3E+00, 1.6E−02; ACIN1, −1.3E+00, 4.1E−02; SERBP1, |
| | −1.3E+00, 2.0E−02; PPP1CC, −1.3E+00, 3.3E−02; NDUFS6, −1.3E+00, 1.6E−02; FLNB, −1.3E+00, 2.7E−02; PPM1H, −1.3E+00, 2.6E−02; TRIM24, −1.3E+00, |
| | 4.6E−02; AIF1L, −1.3E+00, 2.4E−03; SLC3A2, −1.3E+00, 2.4E−02; KIF1A, −1.3E+00, 1.2E−02; ADD2, −1.3E+00, 1.7E−02; COX7C, −1.3E+00, 4.5E−02; TLE1, |
| | −1.3E+00, 5.5E−03; H4C9, −1.3E+00, 4.4E−02; SLC7A5, −1.3E+00, 1.8E−03; HSPA8, −1.3E+00, 1.8E−02; AZIN1, −1.3E+00, 4.0E−02; CALB1, −1.3E+00, 8.3E−05; |
| | RPS8, −1.2E+00, 3.2E−03; ESF1, −1.2E+00, 1.9E−02; METAP2, −1.2E+00, 4.4E−02; SLC29A1, −1.2E+00, 2.1E−03; RBM47, −1.2E+00, 2.0E−02; RPL13A, |
| | −1.2E+00, 6.6E−03; TRIM71, −1.2E+00, 4.6E−02; ZNF770, −1.2E+00, 2.9E−02; PTTG1, −1.2E+00, 2.4E−02; TBCA, −1.2E+00, 4.7E−02; F11R, −1.2E+00, 3.1E−02; |
| | SLC9A3R1, −1.2E+00, 1.7E−03; PABPC1, −1.2E+00, 1.7E−02; ARMCX3, −1.2E+00, 9.2E−04; SYNDIG1, −1.2E+00, 9.1E−03; LINC00428, −1.2E+00, 3.5E−03; |
| | NDUFA3, −1.2E+00, 3.5E−03; H4C12, −1.2E+00, 2.0E−02; PCAT14, −1.2E+00, 4.5E−03; ELOB, −1.2E+00, 3.3E−02; ITM2B, −1.1E+00, 3.7E−02; NDUFB9, |
| | −1.1E+00, 2.9E−02; COX7A2, −1.1E+00, 4.5E−02; SELENOW, −1.1E+00, 4.4E−02; MDH2, −1.1E+00, 6.5E−03; UQCRQ, −1.1E+00, 6.3E−03; TOMM6, −1.1E+00, |
| | 3.2E−02; SORBS1, −1.1E+00, 4.1E−02; TXNDC17, −1.1E+00, 3.2E−02; C12orf60, −1.1E+00, 1.6E−03; LSM3, −1.1E+00, 4.6E−02; COX7B, −1.1E+00, 1.6E−02; |
| | GPX1, −1.1E+00, 1.5E−02; HSP90AB1, −1.1E+00, 5.0E−03; HNRNPA2B1, −1.1E+00, 6.8E−03; ATP6V1G1, −1.1E+00, 4.7E−02; CDCP1, −1.0E+00, 2.5E−02; |
| | GCNT2, −1.0E+00, 4.2E−02; INSR, −1.0E+00, 3.9E−02; ROMO1, −1.0E+00, 4.6E−02; NPM1, −9.9E−01, 2.2E−02; SEC13, −9.9E−01, 2.7E−02; LOC105375710, |
| | −9.8E−01, 1.2E−04; RPA3, −9.7E−01, 2.5E−02; TFAP2C, −9.7E−01, 3.1E−04; SEL1L3, −9.6E−01, 1.9E−02; EEF1A1, −9.4E−01, 3.7E−03; TMEM64, −9.4E−01, 3.9E−02; |
| | CYP2S1, −9.4E−01, 2.7E−02; PAK1IP1, −9.1E−01, 4.4E−02; BEND4, −8.9E−01, 3.2E−03; SCGB3A2, −8.8E−01, 6.0E−04; ICA1, −8.5E−01, 2.2E−02; RAB15, |
| | −8.5E−01, 2.5E−02; FEZ1, −8.4E−01, 1.4E−02; FXYD5, −8.2E−01, 6.6E−03; SF3B4, −8.1E−01, 4.5E−02; ND4, −8.1E−01, 2.9E−04; CD55, −8.0E−01, 1.9E−02; KLKB1, |
| | −7.9E−01, 1.3E−02; MTIF3, −7.8E−01, 1.9E−02; RND2, −7.7E−01, 3.6E−02; CASTOR2, −7.5E−01, 3.8E−02; HLA-C, −7.5E−01, 2.9E−02; RPS14P3, −7.4E−01, 2.8E−02; |
| | CRABP1, −7.3E−01, 2.3E−02; PLAAT3, −7.3E−01, 1.7E−02; PLAAT5, −6.7E−01, 7.1E−03; RHOU, −6.3E−01, 3.0E−02; MUC3A, −6.2E−01, 2.8E−03; FGFR3, |
| | −5.7E−01, 4.5E−02; MBP, −5.5E−01, 9.0E−03; LOC105379087, −5.5E−01, 2.1E−02; NANOG, −5.3E−01, 4.0E−02; PILRA, −5.3E−01, 3.5E−02; NKAIN4, −5.1E−01, |
| | 4.0E−02; GPANK1, −5.1E−01, 1.4E−02; ATP6VOD2, −4.5E−01, 1.4E−02; MIR302C, −3.0E−01, 6.2E−03; FABP4, 2.9E−02, 2.7E−02; LOC101928523, 2.9E−02, |
| | 2.7E−02; LOC102723517, 2.9E−02, 2.7E−02; LOC728084, 2.9E−02, 2.7E−02; LOC105379355, 3.1E−02, 4.0E−02; LOC107985155, 3.1E−02, 4.0E−02; |
| | LOC105370855, 3.3E−02, 2.3E−02; LOC107985001, 3.3E−02, 2.3E−02; SNORA72, 3.3E−02, 2.3E−02; LOC105371498, 3.3E−02, 4.1E−02; GAPLINC, 3.5E−02, |
| | 4.1E−02; LOC105372092, 3.5E−02, 4.1E−02; LOC105373397, 3.5E−02, 4.1E−02; LOC107985569, 3.5E−02, 4.1E−02; TBC1D26, 3.5E−02, 4.1E−02; CD93, |
| | 3.8E−02, 2.4E−02; MIR181A2, 3.8E−02, 2.4E−02; SERPINB3, 3.8E−02, 2.4E−02; LINC01221, 3.8E−02, 4.6E−02; LOC101927829, 3.8E−02, 4.6E−02; |
| | LOC105379091, 3.8E−02, 4.6E−02; OR2A25, 3.8E−02, 4.6E−02; POU6F2-AS2, 3.8E−02, 4.6E−02; TNFSF14, 3.8E−02, 4.6E−02; LOC101928272, 4.2E−02, |
| | 3.8E−02; TMEM211, 4.2E−02, 3.8E−02; C4BPB, 4.3E−02, 1.6E−02; LY6G6C, 4.3E−02, 1.6E−02; LOC105377687, 4.4E−02, 3.0E−02; LOC107986117, 4.4E−02, |
| | 3.0E−02; SNORA11, 4.4E−02, 3.0E−02; LOC107986842, 4.4E−02, 1.9E−02; LOC107987085, 4.4E−02, 1.9E−02; MIR4256, 4.4E−02, 3.1E−02; LOC105369874, |
| | 4.4E−02, 4.1E−02; LOC105376435, 4.4E−02, 4.1E−02; LOC105377706, 4.4E−02, 4.1E−02; AQP7, 4.7E−02, 2.1E−02; CYP2B7P, 4.9E−02, 4.4E−02; DBH, 4.9E−02, |
| | 4.4E−02; LOC105370251, 4.9E−02, 4.4E−02; LOC100130345, 5.0E−02, 3.7E−02; LOC105378400, 5.3E−02, 4.6E−02; RNVU1-19, 5.5E−02, 3.5E−02; |
| | LOC101927740, 5.6E−02, 4.0E−02; LOC107985349, 5.6E−02, 4.5E−02; NANOS3, 6.0E−02, 1.7E−02; LOC728327, 6.1E−02, 9.2E−03; CEP170P1, 6.2E−02, |
| | 3.5E−02; FGF14-IT1, 6.3E−02, 5.8E−03; LOC105373041, 6.4E−02, 2.4E−02; BHMG1, 6.4E−02, 1.5E−02; BMI1, 6.5E−02, 5.7E−03; HLA-H, 6.7E−02, 6.8E−03; |
| | LOC105375085, 6.8E−02, 3.6E−02; LOC105371529, 7.1E−02, 4.6E−02; CISH, 7.2E−02, 2.0E−02; LINC00305, 7.2E−02, 4.0E−03; LOC105372104, 7.3E−02, |
| | 3.3E−02; LOC105377367, 7.3E−02, 2.6E−03; LOC107985284, 7.4E−02, 2.9E−02; LOC105374517, 7.7E−02, 4.1E−02; LOC107987064, 7.9E−02, 2.5E−03; |
| | LOC105377709, 7.9E−02, 1.8E−02; LOC107986013, 8.1E−02, 4.5E−02; SOWAHD, 8.2E−02, 2.3E−02; KCNMB1, 8.3E−02, 1.0E−03; LOC107985458, 8.4E−02, |
| | 2.6E−02; LOC107986771, 8.5E−02, 4.5E−02; SLC5A8, 8.5E−02, 2.4E−02; LOC102723339, 8.5E−02, 3.9E−02; LOC100506474, 8.7E−02, 5.3E−03; |
| | LOC107985504, 8.8E−02, 2.6E−02; PIP, 8.9E−02, 3.7E−03; LINC01491, 8.9E−02, 4.2E−03; LOC105374559, 9.0E−02, 3.1E−02; LOC107986527, 9.4E−02, |
| | 4.4E−02; LINC01152, 9.4E−02, 4.2E−03; LOC101927646, 9.5E−02, 1.4E−03; LOC105371874, 9.7E−02, 2.7E−02; PLXNB3, 9.7E−02, 3.2E−03; XAF1, 9.9E−02, |
| | 1.9E−02; LOC101929717, 1.0E−01, 3.9E−02; LOC105376411, 1.0E−01, 2.1E−02; LOC105378825, 1.0E−01, 4.6E−02; INAFM1, 1.0E−01, 3.5E−02; |
| | LOC105372733, 1.1E−01, 3.2E−02; CPA1, 1.1E−01, 6.8E−03; LOC105374340, 1.1E−01, 4.2E−02; MGC27382, 1.1E−01, 1.1E−03; LOC105373117, 1.1E−01, |
| | 4.1E−02; LOC107986712, 1.1E−01, 4.5E−02; LOC107986600, 1.2E−01, 2.2E−02; ADAP2, 1.2E−01, 7.1E−03; PEAR1, 1.2E−01, 4.4E−02; LOC100128882, |
| | 1.3E−01, 1.4E−04; LOC644135, 1.3E−01, 2.0E−03; DNAI2, 1.3E−01, 4.8E−02; LOC105369428, 1.3E−01, 3.5E−03; LOC105371625, 1.3E−01, 5.0E−03; |
| | LOC107986707, 1.4E−01, 1.3E−02; TMEM14EP, 1.4E−01, 1.4E−02; COL8A2, 1.4E−01, 4.7E−02; THEMIS, 1.5E−01, 5.6E−04; LOC105369890, 1.5E−01, 3.0E−02; |
| | LOC107985779, 1.5E−01, 1.8E−03; LOC107985054, 1.6E−01, 5.9E−05; LOC107986189, 1.6E−01, 3.1E−02; ACVRL1, 1.6E−01, 1.0E−02; LINC01252, |
| | 1.6E−01, 3.6E−02; LOC100130476, 1.6E−01, 1.5E−02; LOC107986028, 1.7E−01, 1.7E−03; LOC100506731, 1.7E−01, 7.0E−05; LOC101928462, 1.7E−01, |
| | 2.8E−02; LOC100507156, 1.7E−01, 3.2E−02; LOC440934, 1.7E−01, 1.5E−03; LOC105378234, 1.7E−01, 3.0E−02; PPP1R3A, 1.7E−01, 1.5E−03; FGF10-AS1, |
| | 1.8E−01, 1.4E−02; LOC105373438, 1.8E−01, 2.2E−03; LOC107984131, 1.8E−01, 2.8E−02; LOC105370604, 1.8E−01, 1.3E−03; GNG8, 1.8E−01, 4.6E−02; |
| | LOC105371303, 1.9E−01, 2.6E−04; LOC101926964, 2.0E−01, 3.3E−03; CCL2, 2.0E−01, 3.9E−02; LOC653602, 2.0E−01, 1.2E−02; LINC00276, 2.0E−01, 7.4E−04; |
| | SCN7A, 2.1E−01, 3.2E−03; LOC105372211, 2.1E−01, 5.4E−03; LOC107984366, 2.1E−01, 4.5E−04; LOC105372550, 2.1E−01, 7.5E−03; LOC105369826, |
| | 2.2E−01, 2.4E−02; CEACAM21, 2.2E−01, 1.7E−02; LINC01389, 2.2E−01, 9.6E−03; LOC107985228, 2.2E−01, 4.6E−02; LOC107985784, 2.3E−01, 1.1E−02; |
| | LINC00877, 2.3E−01, 4.4E−02; NDST4, 2.3E−01, 2.5E−02; TNS2, 2.3E−01, 7.3E−03; LOC107986709, 2.3E−01, 1.1E−02; FAS, 2.3E−01, 5.4E−04; ATOH8, 2.3E−01, |
| | 1.3E−02; HTR1A, 2.3E−01, 2.9E−04; SLC5A9, 2.3E−01, 4.7E−02; LOC105374464, 2.3E−01, 1.5E−03; LOC105377979, 2.4E−01, 9.4E−03; IER5L, 2.4E−01, |
| | 4.7E−04; LOC105377291, 2.4E−01, 1.1E−02; CPED1, 2.5E−01, 6.9E−03; ENG, 2.5E−01, 6.1E−03; ADAMTS9-AS1, 2.5E−01, 1.3E−03; CDRT1, 2.6E−01, 3.0E−04; |
| | GDF6, 2.6E−01, 2.1E−04; VAX1, 2.6E−01, 5.2E−04; A2M, 2.6E−01, 3.9E−02; LOC102723906, 2.6E−01, 1.1E−02; ADGRF2, 2.7E−01, 3.8E−05; RCSD1, 2.7E−01, |
| | 3.5E−04; LOC105372160, 2.7E−01, 3.6E−02; POSTN, 2.7E−01, 2.7E−02; LOC105370555, 2.8E−01, 4.0E−02; LINC00936, 2.8E−01, 3.8E−03; PCDHGC4, |
| | 2.8E−01, 5.6E−03; PIK3C2G, 2.8E−01, 2.5E−02; ITGA11, 2.8E−01, 2.2E−02; HSD17B3, 2.8E−01, 1.8E−02; ABCA10, 2.8E−01, 1.3E−02; ARHGEF15, 2.9E−01, |
| | 4.0E−02; GATA2, 2.9E−01, 1.0E−02; BCHE, 2.9E−01, 4.0E−03; VSTM2A, 2.9E−01, 5.3E−04; SLC8A1-AS1, 3.0E−01, 6.2E−05; LOC105375631, 3.0E−01, 7.9E−03; |
| | LOC105375821, 3.0E−01, 5.7E−03; SYTL4, 3.0E−01, 8.7E−04; KLHDC8B, 3.0E−01, 1.0E−02; CTBS, 3.0E−01, 3.5E−02; EPHB3, 3.1E−01, 9.8E−04; DACT3, |
| | 3.1E−01, 3.0E−04; LOC339622, 3.1E−01, 1.4E−02; LOC105374039, 3.2E−01, 3.1E−05; PRICKLE2-AS1, 3.2E−01, 2.6E−08; LOC105376486, 3.2E−01, 3.5E−02; |
| | SMARCD3, 3.3E−01, 2.3E−02; S1PR1, 3.3E−01, 1.5E−08; BVES, 3.3E−01, 4.6E−02; SMYD1, 3.3E−01, 1.2E−04; RFTN1, 3.3E−01, 1.9E−02; TMEM246, 3.4E−01, |
| | 3.6E−02; ENPEP, 3.4E−01, 6.8E−04; SPX, 3.4E−01, 3.4E−04; MESDC1, 3.4E−01, 3.9E−02; WASH2P, 3.5E−01, 3.5E−03; LOC100506178, 3.5E−01, 4.0E−05; |
| | TRIM32, 3.6E−01, 7.8E−03; KCTD12, 3.7E−01, 1.4E−02; PDZRN4, 3.7E−01, 1.4E−04; LOC105370487, 3.7E−01, 7.0E−03; ELFN1, 3.9E−01, 3.0E−02; TRIL, |
| | 3.9E−01, 4.8E−04; CNTN6, 3.9E−01, 3.9E−04; TRMT10A, 4.0E−01, 2.2E−02; SYTL5, 4.0E−01, 3.8E−02; ADGRA2, 4.1E−01, 2.3E−02; FOXP2, 4.1E−01, 2.8E−02; |
| | CYP7B1, 4.2E−01, 1.5E−02; COL3A1, 4.2E−01, 8.0E−05; SRPX2, 4.2E−01, 1.3E−06; LOC105379055, 4.2E−01, 1.7E−03; COPG2IT1, 4.2E−01, 1.2E−03; DAB2, |
| | 4.3E−01, 2.3E−02; RCAN1, 4.3E−01, 1.4E−02; LOC105374322, 4.3E−01, 1.7E−03; FKBP9, 4.3E−01, 4.1E−02; LOC105374959, 4.3E−01, 2.3E−03; PGM5, 4.4E−01, |
| | 7.0E−08; ADAM12, 4.4E−01, 3.2E−03; LOC151760, 4.4E−01, 7.9E−03; LOC101929710, 4.4E−01, 3.1E−02; MSC, 4.5E−01, 3.2E−03; ZHX1, 4.5E−01, 2.0E−02; |
| | SVEP1, 4.5E−01, 3.4E−07; CLECIA, 4.5E−01, 2.3E−07; LOC107984656, 4.6E−01, 6.9E−03; PCDHGC3, 4.6E−01, 3.0E−02; BLOC1S5−TXNDC5, 4.6E−01, |
| | 3.8E−02; C22orf29, 4.6E−01, 1.9E−02; ASXL3, 4.7E−01, 2.7E−02; IL1R1, 4.8E−01, 6.0E−05; LRRC6, 4.8E−01, 1.3E−02; NFKB1, 4.8E−01, 4.1E−02; P2RY1, |
| | 4.8E−01, 1.9E−02; FSIP2, 4.8E−01, 4.3E−02; SLITRK5, 4.9E−01, 1.2E−05; TMEM169, 4.9E−01, 2.7E−02; CNTN5, 4.9E−01, 2.1E−03; FAM104B, 4.9E−01, 3.3E−02; |
| | GNG12-AS1, 5.0E−01, 2.5E−06; ALX1, 5.0E−01, 2.4E−05; CUEDC1, 5.0E−01, 3.1E−02; SLC16A7, 5.0E−01, 3.1E−02; NTF3, 5.1E−01, 1.2E−02; GNPDA2, |
| | 5.1E−01, 2.2E−02; PLXND1, 5.1E−01, 3.4E−02; RASSF9, 5.1E−01, 3.8E−02; GNB1L, 5.1E−01, 8.8E−04; PKD1L1, 5.1E−01, 1.2E−03; RUNX2, 5.2E−01, 1.2E−04; |
| | ATP7A, 5.2E−01, 1.8E−02; NNMT, 5.2E−01, 1.2E−14; LOC101927815, 5.2E−01, 3.7E−04; MATN3, 5.2E−01, 1.3E−04; KDELC2, 5.2E−01, 1.4E−02; ARSJ, 5.2E−01, |
| | 3.9E−02; LINC01237, 5.2E−01, 4.6E−02; AMER2, 5.2E−01, 2.6E−06; SLFN5, 5.3E−01, 1.0E−03; LOC105369863, 5.3E−01, 3.4E−07; POU3F2, 5.4E−01, |
| | 1.4E−12; RGS3, 5.4E−01, 3.1E−02; NETO1, 5.4E−01, 2.7E−02; BAHCC1, 5.4E−01, 3.4E−06; PAG1, 5.4E−01, 1.7E−06; LOC107985969, 5.4E−01, 4.5E−02; |
| | ANGPT2, 5.4E−01, 6.8E−08; SUSD4, 5.5E−01, 1.3E−03; HECTD2, 5.6E−01, 7.2E−03; LOC101927359, 5.7E−01, 3.8E−07; ST3GAL1, 5.7E−01, 2.4E−03; PALM2− |
| | AKAP2, 5.7E−01, 2.4E−02; STAT6, 5.8E−01, 3.1E−02; BRINP2, 5.8E−01, 1.4E−12; LGALS1, 5.8E−01, 2.0E−03; EHD2, 5.8E−01, 2.4E−02; PDE7B, 5.9E−01, |
| | 1.1E−08; HHAT, 5.9E−01, 3.7E−03; YAF2, 5.9E−01, 1.5E−02; GADD45A, 5.9E−01, 3.3E−03; NDRG1, 6.0E−01, 1.1E−09; LOC101929563, 6.0E−01, 1.9E−02; UNC5C, |
| | 6.0E−01, 4.0E−03; PLAGL1, 6.0E−01, 3.7E−05; KIFAP3, 6.0E−01, 6.6E−03; SHANK1, 6.1E−01, 1.1E−03; DKK2, 6.1E−01, 3.5E−11; HGSNAT, 6.1E−01, 1.0E−02; |
| | LOC105374494, 6.2E−01, 3.9E−02; UBE2J1, 6.2E−01, 2.1E−03; NPR1, 6.3E−01, 2.2E−04; LOC102724832, 6.3E−01, 1.2E−02; WLS, 6.3E−01, 7.0E−03; |
| | TNFRSF19, 6.4E−01, 4.7E−04; SMYD2, 6.4E−01, 1.7E−03; SMAD6, 6.4E−01, 4.6E−02; RNF152, 6.5E−01, 3.2E−03; NID1, 6.6E−01, 1.6E−02; ENOX1, 6.6E−01, |
| | 9.4E−03; TSPAN12, 6.6E−01, 1.4E−02; DNM3OS, 6.6E−01, 6.9E−17; SPSB1, 6.7E−01, 4.9E−04; RNF180, 6.7E−01, 8.1E−03; C12orf4, 6.7E−01, 3.9E−02; |
| | SLC2A14, 6.8E−01, 5.1E−03; CLMN, 6.8E−01, 2.4E−02; EMILIN2, 6.8E−01, 5.6E−03; LOC105373202, 6.8E−01, 3.1E−02; ST8SIA2, 6.8E−01, 9.5E−04; CPE, |
| | 6.9E−01, 6.5E−03; LOC100507291, 6.9E−01, 2.6E−02; MECOM, 7.0E−01, 4.6E−03; ARFGAP3, 7.0E−01, 1.1E−02; ALPK2, 7.0E−01, 6.3E−07; TRIM5, 7.0E−01, |
| | 2.7E−02; MEGF8, 7.0E−01, 1.9E−02; MIR503HG, 7.0E−01, 2.8E−12; TIMP1, 7.1E−01, 3.7E−03; RIN2, 7.1E−01, 1.1E−04; DPY19L1, 7.1E−01, 3.6E−02; SHC3, |
| | 7.1E−01, 1.2E−05; ADAM9, 7.2E−01, 4.3E−02; WIPF1, 7.2E−01, 1.8E−03; BICC1, 7.2E−01, 1.9E−04; SRGAP2C, 7.2E−01, 1.0E−03; RNF19A, 7.2E−01, 1.3E−02; |
| | LINC00152, 7.3E−01, 3.4E−02; CXXC4, 7.3E−01, 4.9E−04; KLF6, 7.3E−01, 2.7E−02; COLGALT2, 7.3E−01, 2.1E−06; TTYH3, 7.4E−01, 3.6E−02; RUSC2, 7.4E−01, |
| | 1.5E−03; LOC100506974, 7.5E−01, 6.6E−04; ARF6, 7.5E−01, 4.1E−02; KBTBD2, 7.5E−01, 2.0E−02; CALCR, 7.5E−01, 6.3E−06; PREX1, 7.5E−01, 2.9E−03; |
| | CSMD3, 7.5E−01, 6.2E−05; JPH2, 7.5E−01, 3.1E−11; MCAM, 7.5E−01, 9.5E−04; FAM155A-IT1, 7.5E−01, 2.9E−07; FBN1, 7.5E−01, 8.6E−04; CYP1B1, 7.6E−01, |
| | 2.3E−10; FAM129B, 7.6E−01, 2.2E−03; MAF, 7.7E−01, 2.2E−03; AFF3, 7.7E−01, 4.4E−05; LRRC8C, 7.8E−01, 5.2E−05; LRRC28, 7.8E−01, 1.1E−02; MRC2, 7.9E−01, |
| | 2.9E−02; EPB41L4A, 7.9E−01, 2.0E−02; SLFN12, 7.9E−01, 1.1E−04; TGFB2, 8.0E−01, 3.8E−09; DUBR, 8.1E−01, 4.5E−09; HEYL, 8.1E−01, 8.0E−04; MMP2, |
| | 8.2E−01, 1.5E−04; TRAM2, 8.2E−01, 1.7E−02; KLF3, 8.3E−01, 3.9E−03; GATA3, 8.3E−01, 8.0E−04; NXPH2, 8.4E−01, 4.0E−02; SLC35F5, 8.4E−01, 3.5E−02; |
| | RGS16, 8.5E−01, 2.5E−02; ZFPM2, 8.5E−01, 1.8E−07; ARHGEF17, 8.5E−01, 1.1E−06; MDGA2, 8.6E−01, 7.0E−03; PLOD1, 8.6E−01, 3.4E−03; GCLM, 8.6E−01, |
| | 2.9E−03; MSRB3, 8.6E−01, 8.2E−08; PKDCC, 8.6E−01, 1.3E−03; MRPS25, 8.7E−01, 2.3E−02; GBE1, 8.7E−01, 1.4E−02; FAR2, 8.8E−01, 1.6E−02; C9orf84, 8.8E−01, |
| | 5.9E−03; HSPA2, 8.8E−01, 3.4E−03; FMNL3, 8.8E−01, 5.9E−03; RBM22, 8.8E−01, 1.8E−03; KCNB2, 8.8E−01, 2.2E−03; LOC105369715, 8.8E−01, 4.7E−15; |
| | NEGR1, 8.8E−01, 4.2E−02; MIR17HG, 8.8E−01, 5.1E−03; MIR181A1HG, 8.9E−01, 2.5E−13; MMP14, 8.9E−01, 4.1E−03; SSH1, 8.9E−01, 2.0E−03; HECW2, |
| | 8.9E−01, 1.2E−02; PPP3CC, 8.9E−01, 2.4E−05; SSFA2, 8.9E−01, 4.5E−07; MLLT11, 8.9E−01, 2.4E−02; CTGF, 9.0E−01, 1.1E−02; PARK2, 9.0E−01, 3.3E−02; |
| | DLC1, 9.1E−01, 3.6E−05; PAPD7, 9.1E−01, 2.4E−03; ARID5B, 9.1E−01, 4.0E−08; ETS1, 9.1E−01, 1.2E−02; KIAA0754, 9.1E−01, 6.6E−04; TNRC18, 9.1E−01, |
| | 3.1E−04; RBMS2, 9.2E−01, 8.0E−04; ARMC9, 9.2E−01, 4.7E−02; NPAS3, 9.2E−01, 3.0E−05; CIRBP, 9.2E−01, 2.2E−02; CREB3L1, 9.3E−01, 1.6E−05; MN1, 9.3E−01, |
| | 1.2E−02; C5orf42, 9.3E−01, 4.8E−02; MIR99AHG, 9.4E−01, 7.5E−09; RUFY2, 9.4E−01, 7.9E−03; RAP1A, 9.5E−01, 3.3E−02; RDH10, 9.5E−01, 6.7E−10; |
| | MAP3K1, 9.5E−01, 1.5E−03; KANSL1L, 9.5E−01, 3.6E−02; GPR161, 9.6E−01, 3.8E−03; PIK3R1, 9.6E−01, 1.2E−03; SEMA5A, 9.6E−01, 3.6E−02; GAB2, 9.6E−01, |
| | 4.0E−03; GLIS3, 9.6E−01, 1.9E−08; GRK5, 9.6E−01, 3.5E−05; DENND2A, 9.7E−01, 2.4E−02; NAALADL2, 9.7E−01, 3.2E−02; SNRPN, 9.7E−01, 4.6E−05; |
| | SPRED2, 9.7E−01, 3.4E−03; MPHOSPH9, 9.8E−01, 3.8E−02; SLC12A2, 9.8E−01, 7.1E−03; KCNH5, 9.8E−01, 1.0E−07; RNF146, 9.9E−01, 1.0E−03; MOB3B, |
| | 9.9E−01, 1.5E−02; RAI1, 9.9E−01, 1.6E−09; MACROH2A1, 1.0E+00, 1.0E−02; RNF144A, 1.0E+00, 1.9E−03; MACROH2A2, 1.0E+00, 3.3E−03; CLIP2, |
| | 1.0E+00, 2.8E−03; FSCN1, 1.0E+00, 4.0E−02; AHI1, 1.0E+00, 1.7E−02; SIK2, 1.0E+00, 7.1E−03; ANKRD26, 1.0E+00, 3.5E−02; NDFIP1, 1.0E+00, 2.2E−03; |
| | SH3PXD2A, 1.0E+00, 1.9E−03; GNAI1, 1.0E+00, 1.9E−02; APBA2, 1.0E+00, 1.7E−04; PRKCE, 1.0E+00, 4.5E−07; ST6GAL2, 1.0E+00, 5.9E−03; PPP4R1, |
| | 1.0E+00, 2.3E−02; ZNF124, 1.0E+00, 4.5E−02; MAN1A2, 1.0E+00, 3.1E−02; DYRK2, 1.0E+00, 4.2E−03; ELL2, 1.1E+00, 1.5E−02; ZBTB18, 1.1E+00, |
| | 1.4E−04; LRFN5, 1.1E+00, 1.1E−11; RCN1, 1.1E+00, 2.6E−02; MAP3K20, 1.1E+00, 7.4E−04; RABGEF1, 1.1E+00, 4.7E−02; STXBP5, 1.1E+00, 2.2E−02; DAAM1, |
| | 1.1E+00, 3.0E−02; DSE, 1.1E+00, 5.9E−03; BNC1, 1.1E+00, 9.4E−05; VOPP1, 1.1E+00, 3.9E−02; ZFYVE16, 1.1E+00, 1.4E−02; F3, 1.1E+00, 1.3E−07; |
| | TBC1D9, 1.1E+00, 7.2E−10; ANTXR2, 1.1E+00, 2.2E−07; RAB28, 1.1E+00, 4.7E−02; AIM2, 1.1E+00, 4.7E−07; WWC3, 1.1E+00, 3.1E−03; TENM1, 1.1E+00, |
| | 3.6E−04; SLC16A12, 1.1E+00, 2.3E−22; NBAS, 1.1E+00, 4.9E−02; MPRIP, 1.1E+00, 2.1E−02; IGF2, 1.1E+00, 8.2E−12; NOL4, 1.1E+00, 6.7E−10; FAT4, |
| | 1.1E+00, 2.8E−09; ATXN1, 1.1E+00, 1.1E−02; RPS6KA3, 1.1E+00, 7.6E−04; TNS1, 1.1E+00, 4.6E−06; ZFP36L1, 1.1E+00, 2.2E−02; BTBD7, 1.1E+00, 3.1E−02; |
| | CRISPLD1, 1.1E+00, 9.2E−03; CENPK, 1.1E+00, 4.2E−02; DGKD, 1.1E+00, 1.4E−06; SCRN1, 1.2E+00, 2.1E−02; PTCHD4, 1.2E+00, 3.4E−07; NUDT4, |
| | 1.2E+00, 1.3E−05; GALNT2, 1.2E+00, 2.6E−02; RNF145, 1.2E+00, 1.7E−03; SMAD5, 1.2E+00, 2.2E−02; GREB1, 1.2E+00, 3.1E−03; SLITRK6, 1.2E+00, |
| | 1.4E−15; ACTG1, 1.2E+00, 4.6E−02; PCDH10, 1.2E+00, 2.0E−10; ARHGAP24, 1.2E+00, 2.3E−05; RIC1, 1.2E+00, 4.6E−03; GLS, 1.2E+00, 1.5E−02; JAM3, 1.2E+00, |
| | 6.6E−03; ANO6, 1.2E+00, 4.1E−02; SDK2, 1.2E+00, 2.4E−02; FBXW8, 1.2E+00, 1.8E−03; DLG5, 1.2E+00, 1.9E−02; OGT, 1.2E+00, 3.3E−02; TTN, 1.2E+00, |
| | 4.8E−02; MEMO1, 1.2E+00, 1.1E−02; ELMO1, 1.2E+00, 5.1E−03; GRB10, 1.2E+00, 2.0E−02; SOX4, 1.2E+00, 4.9E−02; TUBA1A, 1.2E+00, 1.7E−02; SIPA1L2, |
| | 1.2E+00, 4.4E−03; ARHGAP10, 1.2E+00, 8.5E−04; MSC-AS1, 1.2E+00, 3.1E−06; TBC1D22A, 1.2E+00, 3.9E−02; CAPN2, 1.2E+00, 5.7E−07; VGLL4, 1.2E+00, |
| | 4.1E−03; GTDC1, 1.2E+00, 1.2E−02; NELL2, 1.2E+00, 7.2E−03; STC1, 1.2E+00, 6.0E−05; COL11A1, 1.2E+00, 2.9E−03; GNB4, 1.2E+00, 2.9E−04; H19, |
| | 1.2E+00, 3.2E−09; HACE1, 1.2E+00, 2.6E−03; GNG12, 1.2E+00, 1.5E−06; TBCK, 1.2E+00, 1.8E−02; PARP8, 1.2E+00, 3.1E−02; PDS5B, 1.2E+00, 4.5E−02; |
| | WSB1, 1.3E+00, 1.8E−02; PDLIM5, 1.3E+00, 9.2E−03; TP53I11, 1.3E+00, 1.0E−03; ABI2, 1.3E+00, 3.8E−02; NEK7, 1.3E+00, 8.0E−04; TACR3, 1.3E+00, |
| | 1.9E−09; AAK1, 1.3E+00, 3.2E−02; TNFRSF21, 1.3E+00, 2.9E−04; UBE2E3, 1.3E+00, 3.1E−02; SLC10A7, 1.3E+00, 4.7E−07; LCORL, 1.3E+00, 1.9E−02; |
| | EBF2, 1.3E+00, 1.3E−11; MYOF, 1.3E+00, 1.1E−07; KNTC1, 1.3E+00, 2.0E−02; PIBF1, 1.3E+00, 8.1E−03; CEP170, 1.3E+00, 3.3E−02; FADS1, 1.3E+00, 4.5E−02; |
| | RTN4, 1.3E+00, 5.0E−02; CPD, 1.3E+00, 1.9E−03; MSN, 1.3E+00, 1.7E−02; FST, 1.3E+00, 5.7E−07; TUSC3, 1.3E+00, 3.1E−02; UBL3, 1.3E+00, 3.1E−04; |
| | LOC101927483, 1.3E+00, 4.4E−04; NAV1, 1.3E+00, 7.5E−03; EVI5, 1.3E+00, 1.7E−03; RUNX1T1, 1.3E+00, 1.6E−02; UGGT2, 1.3E+00, 3.6E−02; USP3, |
| | 1.3E+00, 3.5E−08; TULP4, 1.3E+00, 2.9E−02; MEF2A, 1.3E+00, 2.7E−02; ELK3, 1.3E+00, 1.0E−06; IGF2BP2, 1.3E+00, 4.9E−02; LOC100506718, 1.3E+00, |
| | 3.6E−21; FAM13A, 1.3E+00, 1.1E−02; FBXW7, 1.3E+00, 2.0E−02; ANGPT1, 1.3E+00, 1.0E−10; METTL15, 1.3E+00, 1.2E−02; MTUS2, 1.3E+00, 8.6E−11; |
| | KIAA1324L, 1.3E+00, 2.6E−03; VPS54, 1.3E+00, 3.3E−03; TTC17, 1.3E+00, 3.9E−02; RNF13, 1.3E+00, 2.6E−03; TNS3, 1.3E+00, 1.1E−02; ITGA1, 1.3E+00, |
| | 9.8E−10; YAP1, 1.3E+00, 3.1E−02; SLIT3, 1.3E+00, 4.2E−05; CEP128, 1.3E+00, 1.5E−02; ABCA5, 1.3E+00, 1.5E−08; DNAJC1, 1.3E+00, 1.7E−04; SFMBT2, |
| | 1.3E+00, 1.5E−03; SCFD2, 1.3E+00, 5.4E−04; HMGCR, 1.3E+00, 4.0E−02; SPATA6, 1.3E+00, 1.6E−04; PHTF2, 1.3E+00, 7.9E−04; COL5A1, 1.3E+00, 6.9E−10; |
| | SLC7A11, 1.3E+00, 3.2E−03; GLCCI1, 1.4E+00, 4.4E−03; TLN1, 1.4E+00, 1.9E−03; PPP3CA, 1.4E+00, 1.5E−02; KLF12, 1.4E+00, 2.0E−02; LYN, 1.4E+00, |
| | 1.2E−03; VTI1A, 1.4E+00, 2.1E−02; SLC8A1, 1.4E+00, 9.6E−10; PELI1, 1.4E+00, 8.0E−03; CORO1C, 1.4E+00, 5.5E−03; EPHA6, 1.4E+00, 1.8E−08; SLC5A3, |
| | 1.4E+00, 8.4E−06; DDR2, 1.4E+00, 1.1E−22; CDC14B, 1.4E+00, 3.0E−03; ADGRG6, 1.4E+00, 3.8E−17; SERPINH1, 1.4E+00, 1.7E−02; ASCC3, 1.4E+00, 3.4E−02; |
| | EPB41L2, 1.4E+00, 1.6E−02; TNIK, 1.4E+00, 1.1E−03; PCNX1, 1.4E+00, 1.0E−02; TEAD1, 1.4E+00, 2.6E−02; ARHGAP6, 1.4E+00, 5.1E−13; MYH9, |
| | 1.4E+00, 1.9E−02; BAZ2B, 1.4E+00, 2.6E−02; BACH1, 1.4E+00, 6.9E−04; SSH2, 1.4E+00, 3.2E−03; PHF14, 1.4E+00, 2.0E−02; RFTN2, 1.4E+00, 6.1E−09; |
| | KIF5B, 1.4E+00, 1.0E−02; TRIM44, 1.4E+00, 1.0E−03; SPATS2, 1.4E+00, 2.0E−03; GPC5, 1.4E+00, 1.4E−11; TRIB2, 1.4E+00, 4.3E−08; NSMCE2, 1.4E+00, |
| | 7.8E−03; HMCN1, 1.4E+00, 5.1E−07; LOC105378178, 1.4E+00, 8.8E−11; PABPC4L, 1.4E+00, 1.2E−17; DCC, 1.5E+00, 6.8E−12; CRIM1, 1.5E+00, 2.4E−03; |
| | FBXL17, 1.5E+00, 2.8E−03; CHSY3, 1.5E+00, 5.4E−08; RFC3, 1.5E+00, 1.4E−02; AGAP1, 1.5E+00, 1.7E−02; RUFY3, 1.5E+00, 1.9E−04; EZH2, 1.5E+00, 4.0E−03; |
| | GREB1L, 1.5E+00, 2.1E−03; ANP32B, 1.5E+00, 6.9E−03; DYRK1A, 1.5E+00, 1.1E−02; FLT1, 1.5E+00, 1.3E−02; CEP85L, 1.5E+00, 2.6E−08; RFX3, |
| | 1.5E+00, 1.1E−02; ITPKB, 1.5E+00, 1.4E−19; MAP1B, 1.5E+00, 1.1E−02; ARHGAP5, 1.5E+00, 5.2E−03; UBR3, 1.5E+00, 3.7E−05; ANKRD17, 1.5E+00, |
| | 1.4E−02; SLC38A2, 1.5E+00, 1.3E−02; GPM6B, 1.5E+00, 1.7E−02; MALAT1, 1.5E+00, 4.4E−13; C1orf21, 1.5E+00, 1.4E−02; CCBE1, 1.5E+00, 2.4E−11; DAAM2, |
| | 1.5E+00, 1.8E−11; WDFY3, 1.5E+00, 9.3E−03; KSR1, 1.5E+00, 7.8E−05; IVNS1ABP, 1.5E+00, 4.5E−03; DIAPH3, 1.5E+00, 1.4E−02; NCOA2, 1.5E+00, |
| | 1.3E−02; MDFIC, 1.5E+00, 2.7E−10; MAN2A1, 1.5E+00, 8.7E−03; LPGAT1, 1.5E+00, 1.8E−09; TMTC2, 1.5E+00, 3.7E−03; FSTL1, 1.5E+00, 9.8E−03; BMPR2, |
| | 1.5E+00, 2.4E−03; KALRN, 1.5E+00, 1.4E−04; ANTXR1, 1.5E+00, 7.7E−04; SMAD2, 1.5E+00, 7.9E−03; RAD54B, 1.5E+00, 3.5E−04; MBD5, 1.5E+00, 9.4E−03; |
| | UHRF1, 1.6E+00, 3.8E−07; PCNX4, 1.6E+00, 4.0E−04; MKLN1, 1.6E+00, 5.1E−03; ST7, 1.6E+00, 1.2E−05; ANXA1, 1.6E+00, 2.0E−18; TRPS1, 1.6E+00, |
| | 3.6E−04; KAT6A, 1.6E+00, 5.7E−04; GBF1, 1.6E+00, 5.9E−05; NR6A1, 1.6E+00, 6.0E−03; PHC2, 1.6E+00, 1.4E−16; GNG2, 1.6E+00, 1.6E−13; |
| | LOC100506885, 1.6E+00, 4.4E−15; LAMB1, 1.6E+00, 9.0E−04; STK3, 1.6E+00, 8.8E−04; AMOTL1, 1.6E+00, 3.4E−05; TENM3, 1.6E+00, 3.2E−03; PDGFRB, |
| | 1.6E+00, 6.3E−18; CAP2, 1.6E+00, 5.3E−06; S1PR3, 1.6E+00, 1.4E−10; HEG1, 1.6E+00, 3.9E−06; MED13, 1.6E+00, 2.8E−03; GSK3B, 1.6E+00, 2.4E−03; |
| | CEP112, 1.6E+00, 1.3E−07; DIP2C, 1.6E+00, 2.7E−04; NR3C1, 1.6E+00, 2.4E−05; LINC01515, 1.7E+00, 1.9E−05; RAD51B, 1.7E+00, 4.4E−03; REEP3, |
| | 1.7E+00, 2.7E−05; TCF4, 1.7E+00, 2.7E−03; MAMDC2, 1.7E+00, 1.6E−09; NAB1, 1.7E+00, 2.6E−10; CCNY, 1.7E+00, 4.2E−04; MED13L, 1.7E+00, 4.4E−03; |
| | PHLDB2, 1.7E+00, 7.4E−05; CYR61, 1.7E+00, 1.5E−09; MBNL2, 1.7E+00, 3.4E−24; PHLPP1, 1.7E+00, 1.7E−03; PPFIBP1, 1.7E+00, 2.0E−05; SPATA5, |
| | 1.7E+00, 1.8E−03; ADAM19, 1.7E+00, 3.8E−10; PRICKLE2, 1.7E+00, 1.0E−05; SRGAP2, 1.7E+00, 5.5E−09; LIN28B, 1.7E+00, 2.4E−03; ADGRL2, 1.7E+00, |
| | 1.7E−03; FIGN, 1.7E+00, 7.9E−04; TSPAN6, 1.7E+00, 2.0E−05; HMGA2, 1.7E+00, 1.3E−03; ZFAND3, 1.7E+00, 2.4E−03; WWC2, 1.7E+00, 1.8E−03; STK38L, |
| | 1.7E+00, 1.2E−06; PTPRG, 1.7E+00, 2.1E−05; TSC22D1, 1.8E+00, 1.5E−03; COLEC12, 1.8E+00, 7.1E−20; ZNF516, 1.8E+00, 3.5E−11; DGKH, 1.8E+00, 4.5E−07; |
| | DOCK4, 1.8E+00, 1.5E−04; EDNRB, 1.8E+00, 3.4E−06; ANO4, 1.8E+00, 4.0E−06; FLNC, 1.8E+00, 2.7E−07; ASAP1, 1.8E+00, 1.0E−03; SDC2, 1.8E+00, |
| | 1.4E−04; MSI2, 1.8E+00, 1.3E−03; TENM2, 1.8E+00, 1.8E−08; ZBTB16, 1.8E+00, 4.0E−21; GNAQ, 1.8E+00, 8.6E−04; VAV3, 1.8E+00, 7.6E−10; COL2A1, |
| | 1.8E+00, 1.6E−15; CACNA1C, 1.8E+00, 8.6E−12; ETV6, 1.9E+00, 3.7E−05; LOC105377134, 1.9E+00, 8.5E−07; PIAS1, 1.9E+00, 3.1E−05; TAGLN, 1.9E+00, |
| | 1.9E−09; MIR924HG, 1.9E+00, 1.5E−03; TBC1D5, 1.9E+00, 3.7E−04; ENC1, 1.9E+00, 3.3E−20; TCF12, 1.9E+00, 4.6E−04; KTN1, 1.9E+00, 2.6E−04; CREB5, |
| | 1.9E+00, 6.9E−21; PKNOX2, 1.9E+00, 1.0E−05; CDK6, 1.9E+00, 1.7E−04; PBX3, 1.9E+00, 5.9E−04; DST, 2.0E+00, 6.3E−05; FTO, 2.0E+00, 1.9E−05; |
| | FERMT2, 2.0E+00, 1.5E−05; CCDC50, 2.0E+00, 2.6E−11; DENND2B, 2.0E+00, 1.4E−09; MEF2C, 2.0E+00, 3.2E−42; APP, 2.0E+00, 9.1E−05; LOC105374693, |
| | 2.0E+00, 5.2E−11; MPDZ, 2.0E+00, 2.7E−06; SMYD3, 2.0E+00, 2.1E−04; PDE10A, 2.0E+00, 9.2E−11; NCALD, 2.1E+00, 6.4E−11; FNDC3B, 2.1E+00, 8.6E−05; |
| | FN1, 2.1E+00, 1.3E−05; RASAL2, 2.1E+00, 7.5E−05; ANKRD1, 2.1E+00, 8.4E−16; NOVA1-AS1, 2.1E+00, 2.7E−18; PLK2, 2.1E+00, 1.0E−29; RASA3, |
| | 2.1E+00, 5.0E−15; LOC107984581, 2.1E+00, 6.1E−17; ROCK2, 2.1E+00, 2.7E−06; LOC644919, 2.1E+00, 3.8E−05; JMJD1C, 2.1E+00, 3.4E−06; PTPRD, |
| | 2.1E+00, 2.9E−05; SETBP1, 2.1E+00, 1.2E−06; FGF10, 2.1E+00, 2.3E−41; SEMA6D, 2.1E+00, 3.3E−11; MAPK10, 2.1E+00, 4.8E−07; AKAP13, 2.1E+00, 2.1E−05; |
| | TLE4, 2.1E+00, 1.5E−06; PTK2, 2.1E+00, 2.7E−05; EPHA7, 2.2E+00, 3.7E−13; COL1A2, 2.2E+00, 9.6E−08; CBLB, 2.2E+00, 2.2E−06; CERS6, 2.2E+00, |
| | 3.1E−06; ANK2, 2.2E+00, 5.5E−07; CLSTN2, 2.2E+00, 7.8E−16; ROR2, 2.2E+00, 6.3E−12; NF1, 2.2E+00, 6.3E−06; TMCC1, 2.2E+00, 1.3E−06; OXR1, |
| | 2.2E+00, 7.3E−13; NEXN, 2.2E+00, 3.9E−09; EDIL3, 2.2E+00, 1.2E−07; THBS1, 2.3E+00, 3.5E−17; RNF217, 2.3E+00, 8.3E−14; PTPRK, 2.3E+00, 5.4E−06; |
| | C3orf70, 2.3E+00, 2.3E−20; FRMD6, 2.3E+00, 6.5E−18; CDH11, 2.3E+00, 7.7E−10; NRIP1, 2.3E+00, 1.2E−10; PARD3, 2.4E+00, 2.1E−06; TRIO, 2.4E+00, |
| | 3.2E−06; NFAT5, 2.4E+00, 7.0E−08; PTPRM, 2.4E+00, 5.8E−05; SCMH1, 2.4E+00, 1.1E−10; COL4A2, 2.4E+00, 8.9E−13; NEAT1, 2.4E+00, 1.6E−09; LHFP, |
| | 2.4E+00, 1.8E−09; PCDH7, 2.4E+00, 2.0E−06; HACD2, 2.4E+00, 1.5E−13; COL4A1, 2.4E+00, 7.3E−13; HDAC9, 2.4E+00, 8.6E−11; BGN, 2.4E+00, 8.2E−30; |
| | SVIL, 2.5E+00, 7.1E−11; LOXL2, 2.5E+00, 4.7E−14; AKT3, 2.5E+00, 5.1E−07; EPHA3, 2.5E+00, 9.6E−24; NID2, 2.5E+00, 8.0E−31; TTC28, 2.5E+00, 6.0E−07; |
| | S100A11, 2.5E+00, 1.9E−10; LINC-PINT, 2.5E+00, 2.6E−13; COL5A2, 2.6E+00, 3.1E−19; EFR3A, 2.6E+00, 2.3E−10; PICALM, 2.6E+00, 2.7E−10; MGAT5, |
| | 2.6E+00, 1.0E−07; BZW2, 2.6E+00, 6.7E−12; SPARC, 2.6E+00, 1.6E−08; ROBO2, 2.7E+00, 3.3E−06; KCNE5, 2.7E+00, 1.3E−47; HS3ST3A1, 2.7E+00, 3.5E−25; |
| | PLEKHA5, 2.7E+00, 1.3E−09; COL12A1, 2.7E+00, 5.8E−59; KCNT2, 2.7E+00, 1.0E−08; SEPTIN7, 2.7E+00, 5.0E−09; ZNF704, 2.8E+00, 2.8E−16; ESRRG, |
| | 2.8E+00, 9.3E−34; WWOX, 2.8E+00, 3.5E−08; SMURF2, 2.8E+00, 1.1E−18; FBXL7, 2.8E+00, 2.2E−09; HEY1, 2.8E+00, 6.0E−58; KIRREL1, 2.8E+00, 5.0E−13; |
| | SNAI2, 2.8E+00, 6.3E−15; PTPN14, 2.8E+00, 1.6E−09; ZSWIM6, 2.8E+00, 2.1E−10; CNTNAP2, 2.8E+00, 3.7E−09; BCAT1, 2.9E+00, 9.8E−10; ZEB1, |
| | 2.9E+00, 2.1E−32; SHROOM3, 2.9E+00, 3.8E−14; HS6ST2, 2.9E+00, 2.3E−10; ST8SIA4, 2.9E+00, 7.7E−75; CHD7, 2.9E+00, 2.7E−10; MID1, 2.9E+00, 6.5E−12; |
| | ZNF521, 3.0E+00, 3.1E−14; COL1A1, 3.0E+00, 2.5E−14; PELI2, 3.0E+00, 2.4E−12; SULF1, 3.0E+00, 8.9E−28; KCND2, 3.1E+00, 1.9E−09; PDE3B, |
| | 3.1E+00, 1.9E−19; LM03, 3.1E+00, 3.6E−39; AHNAK, 3.1E+00, 6.0E−17; TANC1, 3.1E+00, 3.4E−22; TFPI, 3.1E+00, 2.9E−58; TTC3, 3.2E+00, 3.1E−16; |
| | RBMS1, 3.2E+00, 1.6E−12; EOGT, 3.2E+00, 2.8E−32; DUSP6, 3.3E+00, 4.6E−34; DDAH1, 3.3E+00, 1.6E−30; CAMK2D, 3.4E+00, 2.6E−22; SPATS2L, |
| | 3.4E+00, 7.3E−15; ST6GALNAC3, 3.4E+00, 3.4E−11; KIF26B, 3.5E+00, 9.2E−16; NES, 3.5E+00, 1.4E−13; FAM126A, 3.5E+00, 9.0E−15; ALCAM, 3.5E+00, |
| | 4.8E−15; CADM1, 3.5E+00, 3.1E−20; ZBTB7C, 3.5E+00, 3.2E−53; ZEB2, 3.6E+00, 8.3E−37; AUTS2, 3.6E+00, 5.0E−19; GATM, 3.6E+00, 1.1E−56; EXT1, |
| | 3.6E+00, 1.4E−14; DOK6, 3.7E+00, 5.8E−33; MACF1, 3.7E+00, 4.4E−21; TBX15, 3.7E+00, 8.9E−39; CCND2, 3.7E+00, 7.9E−16; PALLD, 3.8E+00, 1.3E−20; |
| | EFNA5, 3.8E+00, 8.4E−17; ARHGAP28, 3.8E+00, 4.9E−27; FAM171A1, 3.8E+00, 2.2E−26; COPG2, 3.9E+00, 1.3E−20; LRP1B, 3.9E+00, 1.7E−44; LPP, |
| | 4.0E+00, 2.8E−18; SSBP2, 4.0E+00, 3.1E−18; NLGN1, 4.1E+00, 6.6E−16; CDK14, 4.1E+00, 1.7E−18; ROBO1, 4.1E+00, 2.0E−18; SAMD4A, 4.1E+00, |
| | 1.1E−34; MIR4435−2HG, 4.1E+00, 2.3E−26; ITGAV, 4.1E+00, 3.0E−34; PARVA, 4.2E+00, 5.0E−39; PRKG1, 4.2E+00, 2.8E−19; ADAMTS9, 4.2E+00, 2.2E−61; P3H2, |
| | 4.4E+00, 4.1E−26; ITPR2, 4.4E+00, 4.6E−27; APBB2, 4.5E+00, 5.0E−24; SOX5, 4.5E+00, 1.4E−22; CDH10, 4.6E+00, 1.8E−53; CCDC102B, 4.7E+00, 6.2E−67; |
| | ARHGAP42, 4.7E+00, 2.9E−28; PLXNA2, 4.7E+00, 8.0E−55; GPC3, 4.8E+00, 1.3E−25; BMPR1B, 4.8E+00, 1.4E−71; EBF1, 4.9E+00, 3.9E−104; MEST, |
| | 4.9E+00, 7.3E−34; CALD1, 5.0E+00, 2.2E−27; PDZD2, 5.2E+00, 7.9E−35; SYT1, 5.2E+00, 2.1E−30; FAT1, 5.3E+00, 1.1E−35; MAML2, 5.3E+00, 2.3E−41; |
| | FBN2, 5.4E+00, 7.8E−46; STXBP5L, 5.4E+00, 2.4E−41; TMTC1, 5.5E+00, 3.8E−33; ITPR1, 5.8E+00, 7.3E−93; GPC6, 5.8E+00, 5.4E−34; PRTG, 6.1E+00, 8.3E−37; |
| | SOX6, 6.2E+00, 1.5E−109; HAPLN1, 6.5E+00, 2.0E−44; SORBS2, 6.6E+00, 3.1E−73; INPP4B, 6.6E+00, 3.1E−100; SEPTIN11, 6.7E+00, 6.4E−50; SLIT2, |
| | 7.0E+00, 4.5E−51; FAM155A, 7.6E+00, 1.5E−65; RBMS3, 8.2E+00, 5.0E−93; RHOBTB3, 8.7E+00, 3.9E−129; ATP2B1, 9.0E+00, 1.1E−94; FLRT2, 9.8E+00, |
| | 5.1E−136; VIM, 9.8E+00, 1.8E−110; ZNF804A, 1.7E+01, 8.5E−284 |
| 6 | PTMA, −3.3E+01, 3.2E−62; RPL4, −3.3E+01, 5.4E−33; RPS8, −3.2E+01, 6.6E−39; ACTG1, −3.2E+01, 7.6E−30; ACTB, −3.2E+01, 6.6E−39; RPL11, −3.1E+01, |
| | 7.0E−33; GAPDH, −3.1E+01, 4.6E−36; RPL6, −3.0E+01, 6.7E−27; EEF1A1, −3.0E+01, 1.9E−58; TPT1, −3.0E+01, 5.0E−24; RPL8, −2.9E+01, 4.8E−23; RPL13A, |
| | −2.9E+01, 6.8E−32; RPL15, −2.9E+01, 1.1E−21; TMSB4X, −2.8E+01, 1.6E−20; RPS24, −2.8E+01, 2.6E−21; RPS19, −2.8E+01, 2.3E−21; HSP90AB1, −2.8E+01, |
| | 3.9E−39; RPL34, −2.8E+01, 1.4E−21; RPL19, −2.8E+01, 5.0E−21; RPL31, −2.8E+01, 3.3E−22; RPS12, −2.7E+01, 4.3E−20; RPS11, −2.7E+01, 1.6E−19; RPL13, |
| | −2.7E+01, 3.9E−20; HNRNPA1, −2.7E+01, 6.7E−30; NPM1, −2.7E+01, 3.9E−35; RPL3, −2.7E+01, 2.9E−23; RPS6, −2.6E+01, 8.9E−31; H4C3, −2.6E+01, 3.4E−15; |
| | LIN28A, −2.6E+01, 9.0E−26; CD24, −2.6E+01, 9.4E−23; RPL7, −2.5E+01, 2.4E−17; RPL14, −2.5E+01, 8.1E−17; EEF2, −2.5E+01, 7.9E−17; RPL18, |
| | −2.5E+01, 3.8E−16; RPS3, −2.4E+01, 1.4E−15; RPL23, −2.4E+01, 1.8E−15; HSP90AA1, −2.4E+01, 9.0E−29; EIF4G2, −2.4E+01, 3.6E−16; FTH1, −2.4E+01, |
| | 1.3E−14; HSPD1, −2.4E+01, 6.8E−19; RPL10A, −2.3E+01, 1.2E−14; RPS2, −2.3E+01, 1.2E−14; RPS23, −2.3E+01, 3.9E−15; RPLP1, −2.3E+01, 5.6E−21; LDHB, |
| | −2.3E+01, 2.9E−15; RPS18, −2.3E+01, 3.8E−16; RPS27A, −2.3E+01, 7.6E−15; SET, −2.3E+01, 4.0E−16; RPL21, −2.3E+01, 1.2E−13; RPL10, −2.3E+01, 7.0E−14; |
| | CANX, −2.2E+01, 4.9E−14; RPLPO, −2.2E+01, 3.0E−13; RPL12, −2.2E+01, 9.8E−14; RACK1, −2.2E+01, 1.5E−13; RPL7A, −2.2E+01, 4.1E−14; RPS7, −2.2E+01, |
| | 7.4E−13; RPS16, −2.2E+01, 8.5E−13; POU5F1, −2.2E+01, 1.5E−12; RPL24, −2.2E+01, 1.6E−12; RPS13, −2.2E+01, 6.3E−13; NCL, −2.2E+01, 3.4E−19; RPS15A, |
| | −2.2E+01, 1.5E−12; PFN1, −2.2E+01, 3.7E−12; RPS27, −2.2E+01, 3.3E−12; GSTP1, −2.2E+01, 5.0E−12; TUBA1B, −2.1E+01, 1.4E−12; RPS15, −2.1E+01, |
| | 1.6E−11; PABPC1, −2.1E+01, 7.2E−16; RPL32, −2.1E+01, 9.6E−12; TUBB, −2.1E+01, 2.3E−12; RPSA, −2.1E+01, 2.6E−11; PAICS, −2.1E+01, 9.3E−12; FTL, −2.1E+01, |
| | 2.1E−11; HMGA1, −2.1E+01, 4.6E−12; NUCKS1, −2.1E+01, 1.2E−13; PRDX1, −2.1E+01, 9.8E−12; H1-5, −2.0E+01, 1.3E−09; CALM1, −2.0E+01, 4.8E−11; |
| | RPL27A, −2.0E+01, 2.1E−11; RPL18A, −2.0E+01, 1.1E−11; NACA, −2.0E+01, 1.1E−10; HNRNPK, −2.0E+01, 4.0E−11; RPS9, −2.0E+01, 1.2E−10; RPS29, |
| | −2.0E+01, 1.4E−10; RPL5, −2.0E+01, 1.7E−11; RPLP2, −2.0E+01, 3.5E−10; TPM3, −2.0E+01, 2.4E−10; ENO1, −2.0E+01, 8.7E−11; RAN, −2.0E+01, 2.2E−10; |
| | NASP, −2.0E+01, 8.0E−12; RPL35A, −2.0E+01, 1.9E−09; RPL26, −1.9E+01, 2.7E−10; HNRNPA2B1, −1.9E+01, 3.4E−19; MDK, −1.9E+01, 3.1E−10; RPL27, |
| | −1.9E+01, 3.0E−11; RPS14, −1.9E+01, 5.0E−10; HSPA8, −1.9E+01, 2.6E−12; MYL6, −1.9E+01, 7.7E−10; CALM2, −1.9E+01, 2.3E−09; RPS4X, −1.9E+01, 2.9E−09; |
| | RPL37, −1.9E+01, 2.6E−09; RPL28, −1.9E+01, 2.1E−09; RPS3A, −1.9E+01, 2.6E−09; EEF1G, −1.8E+01, 5.1E−09; YWHAE, −1.8E+01, 4.7E−09; UBB, |
| | −1.8E+01, 2.9E−09; TMSB10, −1.8E+01, 1.2E−09; SCD, −1.8E+01, 2.4E−09; RPS17, −1.8E+01, 2.7E−08; HMGB2, −1.8E+01, 3.1E−09; GDI2, −1.7E+01, 2.2E−08; |
| | CBX5, −1.7E+01, 2.3E−08; CEBPZ, −1.7E+01, 4.4E−08; CHD4, −1.7E+01, 5.8E−08; PRDX6, −1.7E+01, 1.2E−08; RPL29, −1.7E+01, 4.5E−08; CFL1, −1.7E+01, |
| | 1.1E−08; HSPA4, −1.7E+01, 7.4E−08; TCP1, −1.7E+01, 1.7E−08; EEF1B2, −1.7E+01, 4.4E−08; SERBP1, −1.7E+01, 2.8E−08; YWHAZ, −1.7E+01, 5.6E−08; |
| | RPS20, −1.7E+01, 1.8E−07; FAU, −1.7E+01, 7.5E−08; RPL36, −1.7E+01, 2.4E−07; TPI1, −1.7E+01, 7.9E−08; CLTC, −1.7E+01, 1.9E−07; RPL9, −1.7E+01, |
| | 1.9E−07; RHOA, −1.7E+01, 1.8E−08; PEBP1, −1.6E+01, 1.5E−08; RPL30, −1.6E+01, 2.8E−07; RPS28, −1.6E+01, 4.4E−08; HMGB1, −1.6E+01, 5.7E−07; YWHAB, |
| | −1.6E+01, 1.7E−07; ATP5F1A, −1.6E+01, 3.2E−07; CCT3, −1.6E+01, 2.7E−07; RPL35, −1.6E+01, 3.1E−07; H2AZ1, −1.6E+01, 7.0E−07; H1-3, −1.6E+01, 3.9E−06; |
| | RPL38, −1.6E+01, 7.6E−08; PTGES3, −1.6E+01, 9.5E−07; STMN1, −1.6E+01, 1.5E−07; SMARCA5, −1.6E+01, 9.0E−07; KPNB1, −1.6E+01, 7.1E−07; |
| | ATP5F1B, −1.6E+01, 1.2E−06; HMGCS1, −1.6E+01, 2.6E−06; SSB, −1.6E+01, 6.4E−07; SOX2, −1.6E+01, 6.2E−07; SKP1, −1.6E+01, 1.3E−07; CALR, −1.5E+01, |
| | 1.7E−06; EIF4B, −1.5E+01, 3.1E−06; YBX1, −1.5E+01, 2.2E−06; MORF4L1, −1.5E+01, 2.5E−06; PARP1, −1.5E+01, 2.0E−06; MATR3, −1.5E+01, 7.1E−07; |
| | SUMO2, −1.5E+01, 8.5E−08; VCP, −1.5E+01, 9.9E−07; CYCS, −1.5E+01, 1.6E−07; XRCC5, −1.5E+01, 2.2E−07; PPIA, −1.5E+01, 3.9E−06; TDGF1, −1.5E+01, |
| | 4.6E−07; G3BP2, −1.5E+01, 8.0E−06; NORAD, −1.5E+01, 2.3E−06; PGRMC1, −1.5E+01, 2.5E−06; RPS5, −1.5E+01, 9.0E−06; CYP51A1, −1.5E+01, 2.2E−06; |
| | GJA1, −1.5E+01, 9.2E−06; CSDE1, −1.5E+01, 9.5E−06; CCT8, −1.5E+01, 2.5E−06; ND5, −1.4E+01, 2.4E−20; H3F3B, −1.4E+01, 8.6E−06; DNMT3B, −1.4E+01, |
| | 4.8E−09; TXNRD1, −1.4E+01, 9.0E−06; COX4I1, −1.4E+01, 4.3E−07; RPL22, −1.4E+01, 1.0E−06; HNRNPA3, −1.4E+01, 2.2E−05; H1-2, −1.4E+01, 1.2E−05; |
| | H2AC17,−1.4E+01, 1.4E−06; HMGB3, −1.4E+01, 4.6E−06; HNRNPU, −1.4E+01, 2.8E−06; ILF3, −1.4E+01, 3.6E−05; IPO5, −1.4E+01, 3.0E−05; ESRG, −1.4E+01, |
| | 7.0E−05; GNAS, −1.4E+01, 4.3E−05; RPS26, −1.4E+01, 5.8E−07; ANP32E, −1.4E+01, 3.4E−06; PLS3, −1.4E+01, 4.2E−05; DYNLL1, −1.4E+01, 5.9E−06; MDH1, |
| | −1.4E+01, 1.5E−05; AP2M1, −1.4E+01, 6.6E−07; RCC2, −1.4E+01, 6.1E−05; SON, −1.4E+01, 6.7E−05; SRSF3, −1.3E+01, 6.1E−05; CCT6A, −1.3E+01, 3.9E−05; |
| | CLDN6, −1.3E+01, 1.5E−05; COX3, −1.3E+01, 7.4E−20; ND6, −1.3E+01, 4.7E−06; UBC, −1.3E+01, 1.1E−05; HMGN1, −1.3E+01, 2.7E−05; CBX3, −1.3E+01, 2.1E−05; |
| | UQCRB, −1.3E+01, 1.1E−06; PDIA3, −1.3E+01, 1.3E−04; ANP32B, −1.3E+01, 5.6E−07; CYTB, −1.3E+01, 7.6E−15; SRP14, −1.3E+01, 1.4E−05; HSP90B1, |
| | −1.3E+01, 2.0E−05; CCT4, −1.3E+01, 7.3E−05; KPNA2, −1.3E+01, 1.4E−04; LITAF, −1.3E+01, 1.1E−05; UBA52, −1.3E+01, 3.8E−05; RPS25, −1.3E+01, 6.7E−06; |
| | CCT5, −1.3E+01, 1.2E−04; H2AC20, −1.3E+01, 5.2E−06; HNRNPM, −1.3E+01, 3.1E−04; L1TD1, −1.3E+01, 7.6E−07; CCND1, −1.3E+01, 1.7E−04; CCT2, |
| | −1.3E+01, 1.4E−04; NONO, −1.3E+01, 2.9E−04; NDUFA4, −1.3E+01, 2.1E−05; DDX18, −1.2E+01, 8.9E−06; RAD21, −1.2E+01, 2.0E−04; DDX1, −1.2E+01, 6.1E−05; |
| | C11orf58, −1.2E+01, 1.1E−04; ACLY, −1.2E+01, 2.4E−04; FKBP3, −1.2E+01, 1.4E−04; HIST1H1E, −1.2E+01, 1.8E−04; NARS1, −1.2E+01, 6.5E−05; PARK7, |
| | −1.2E+01, 6.7E−05; COX6B1, −1.2E+01, 2.9E−07; TKT, −1.2E+01, 3.7E−04; DDX3X, −1.2E+01, 3.1E−04; CLIC4, −1.2E+01, 7.6E−04; BTF3, −1.2E+01, 1.5E−04; |
| | APEX1, −1.2E+01, 4.0E−05; DBI, −1.2E+01, 2.6E−05; AKIRIN1, −1.2E+01, 4.5E−04; SUPT16H, −1.2E+01, 8.4E−04; XRCC6, −1.2E+01, 8.4E−04; TARS1, |
| | −1.2E+01, 1.3E−03; HNRNPDL, −1.2E+01, 8.8E−04; RPL37A, −1.2E+01, 1.2E−03; ATP5PD, −1.2E+01, 1.8E−05; SF3B1, −1.2E+01, 1.2E−03; DNAJA1, −1.2E+01, |
| | 2.5E−04; MSH6, −1.2E+01, 1.1E−03; COX5B, −1.2E+01, 1.7E−05; IQGAP1, −1.2E+01, 9.4E−04; RPS21, −1.2E+01, 7.4E−04; YWHAG, −1.2E+01, 7.7E−04; SLIRP, |
| | −1.2E+01, 8.8E−05; ST13, −1.1E+01, 2.9E−04; OAZ1, −1.1E+01, 5.9E−05; CCT7, −1.1E+01, 1.9E−05; DDX5, −1.1E+01, 2.5E−03; HSPA5, −1.1E+01, 2.0E−03; |
| | CDC42, −1.1E+01, 1.0E−04; CSE1L, −1.1E+01, 1.5E−03; IGF2BP1, −1.1E+01, 1.4E−03; SMC3, −1.1E+01, 1.7E−03; COX7C, −1.1E+01, 8.5E−04; RBMX, |
| | −1.1E+01, 1.4E−03; TRIM28, −1.1E+01, 1.8E−04; EIF1, −1.1E+01, 1.1E−04; PODXL, −1.1E+01, 8.3E−04; IFITM1, −1.1E+01, 3.7E−06; ARL6IP1, −1.1E+01, 7.7E−04; |
| | DSTN, −1.1E+01, 8.0E−04; SNHG5, −1.1E+01, 1.7E−04; RPL23A, −1.1E+01, 2.4E−04; H2AZ2, −1.1E+01, 4.0E−05; FLNA, −1.1E+01, 2.7E−03; SERF2, |
| | −1.1E+01, 7.7E−05; NOLC1, −1.1E+01, 8.8E−04; UGP2, −1.1E+01, 3.9E−04; DEK, −1.1E+01, 4.0E−03; PSMA4, −1.1E+01, 8.1E−04; EIF2S3, −1.1E+01, 2.3E−03; |
| | UBTF, −1.0E+01, 2.9E−05; SEPHS1, −1.0E+01, 3.3E−03; DENR, −1.0E+01, 1.4E−03; MCM4, −1.0E+01, 5.9E−03; RN7SK, −1.0E+01, 3.6E−03; ALDOA, −1.0E+01, |
| | 5.9E−04; CCNB1, −1.0E+01, 7.8E−04; TARDBP, −1.0E+01, 2.0E−03; HSBP1, −1.0E+01, 8.7E−05; LDHA, −1.0E+01, 4.7E−05; CD63, −1.0E+01, 5.3E−04; COX1, |
| | −1.0E+01, 1.1E−14; HMGN2, −1.0E+01, 1.4E−03; AZIN1, −1.0E+01, 2.4E−03; PEG10, −1.0E+01, 6.8E−04; H2AC11, −1.0E+01, 1.7E−04; NDUFS5, −1.0E+01, |
| | 1.4E−03; TOMM20, −1.0E+01, 2.5E−03; SRRM2, −1.0E+01, 7.5E−03; TFAM, −1.0E+01, 3.2E−04; ACVR2B, −1.0E+01, 4.2E−03; NUDT21, −1.0E+01, 8.8E−04; |
| | SNRNP200, −1.0E+01, 3.1E−03; STIP1, −1.0E+01, 2.5E−03; CLU, −1.0E+01, 1.6E−03; MAPRE1, −9.9E+00, 6.9E−04; G3BP1, −9.9E+00, 1.2E−02; RANBP2, |
| | −9.9E+00, 1.1E−02; SERPINB9, −9.8E+00, 8.8E−04; SKIL, −9.8E+00, 1.3E−02; NAP1L1, −9.8E+00, 5.0E−03; DCP2, −9.8E+00, 8.2E−03; ABCF1, −9.8E+00, 7.7E−04; |
| | MCM6, −9.8E+00, 2.4E−03; PKM, −9.7E+00, 6.2E−03; HDLBP, −9.7E+00, 1.3E−02; PDIA6, −9.7E+00, 9.4E−03; ATP6, −9.7E+00, 4.4E−09; PPP1CC, |
| | −9.7E+00, 6.2E−03; ND1, −9.7E+00, 3.3E−04; SOD1, −9.7E+00, 1.5E−04; TMED2, −9.7E+00, 7.8E−04; COX2, −9.6E+00, 3.7E−06; HNRNPD, −9.6E+00, 1.6E−02; |
| | ARPC2, −9.6E+00, 3.1E−03; PRRC2A, −9.6E+00, 1.5E−03; CNOT1, −9.6E+00, 1.3E−02; YWHAQ, −9.5E+00, 1.6E−02; TOP2A, −9.5E+00, 3.0E−02; MLEC, |
| | −9.5E+00, 1.1E−02; ND4L, −9.5E+00, 1.3E−02; SLC16A1, −9.5E+00, 1.6E−02; MTHFD2, −9.5E+00, 1.8E−03; HNRNPR, −9.5E+00, 1.3E−02; CNBP, −9.4E+00, |
| | 7.7E−03; TUBB2B, −9.4E+00, 1.0E−04; PSIP1, −9.4E+00, 2.0E−02; KRT18, −9.4E+00, 6.1E−05; SRSF7, −9.4E+00, 5.0E−03; KRT8, −9.3E+00, 2.4E−04; FBL, |
| | −9.3E+00, 2.0E−03; SLC2A3, −9.3E+00, 9.4E−04; GSPT1, −9.3E+00, 1.0E−03; RBM3, −9.3E+00, 6.2E−04; PDIA4, −9.3E+00, 7.9E−03; YBX3, −9.2E+00, 1.8E−03; |
| | ERH, −9.2E+00, 1.6E−03; SERPINH1, −9.2E+00, 3.2E−03; DPPA4, −9.1E+00, 1.7E−02; H3C4, −9.1E+00, 1.3E−03; EZR, −9.1E+00, 2.2E−02; ACTR2, −9.1E+00, |
| | 1.0E−02; PSME3, −9.1E+00, 3.3E−04; ZNF770, −9.1E+00, 8.0E−03; RSL1D1, −9.1E+00, 1.1E−02; TPM4, −9.0E+00, 2.2E−02; GLO1, −9.0E+00, 5.8E−03; ATP8, |
| | −9.0E+00, 1.8E−02; ND2, −9.0E+00, 7.5E−04; C14orf166, −9.0E+00, 6.4E−03; EIF2S2, −9.0E+00, 6.3E−03; CFAP298, −9.0E+00, 2.8E−03; TUBA1A, −8.9E+00, |
| | 1.1E−03; SEPTIN2, −8.9E+00, 3.0E−02; MARCKS, −8.9E+00, 2.5E−03; UBA2, −8.9E+00, 1.1E−02; NFE2L1, −8.9E+00, 5.6E−03; PSMB1, −8.9E+00, 3.0E−03; |
| | PABPC4, −8.9E+00, 1.2E−03; SOX11, −8.9E+00, 6.7E−03; BZW1, −8.9E+00, 2.0E−03; THRAP3, −8.8E+00, 2.9E−02; ND4, −8.8E+00, 1.6E−13; RPL7L1, |
| | −8.8E+00, 1.1E−03; PAIP2, −8.8E+00, 6.0E−03; PGD, −8.8E+00, 3.7E−03; SOX4, −8.8E+00, 1.8E−03; FSCN1, −8.7E+00, 1.2E−04; SUB1, −8.7E+00, 6.4E−03; |
| | PPAT, −8.7E+00, 5.4E−03; HSPA9, −8.7E+00, 3.4E−02; IDH1, −8.7E+00, 3.6E−02; ATP5PB, −8.7E+00, 2.9E−03; CKB, −8.7E+00, 8.4E−04; TMA7, −8.6E+00, |
| | 2.8E−04; BEX3, −8.6E+00, 1.4E−02; SELENOW, −8.6E+00, 1.0E−03; ILF2, −8.6E+00, 6.4E−03; H3C2, −8.6E+00, 1.3E−02; ZFP42, −8.6E+00, 1.2E−02; MYH10, |
| | −8.6E+00, 1.6E−02; FDFT1, −8.6E+00, 4.4E−02; SFRP1, −8.6E+00, 3.6E−02; CCNG1, −8.6E+00, 2.9E−02; JPT2, −8.5E+00, 2.0E−02; ATP5MG, −8.5E+00, |
| | 1.2E−03; PTBP1, −8.5E+00, 1.8E−02; UBE21, −8.5E+00, 4.4E−03; H3C6, −8.5E+00, 1.7E−04; FUS, −8.5E+00, 4.2E−02; APLP2, −8.5E+00, 4.3E−02; EIF5A, −8.4E+00, |
| | 2.0E−02; PSMB4, −8.4E+00, 6.1E−04; DUT, −8.4E+00, 6.9E−03; USP7, −8.4E+00, 2.4E−02; RPL41, −8.4E+00, 2.6E−03; SARAF, −8.4E+00, 2.1E−03; NUDC, |
| | −8.4E+00, 6.2E−03; LTA4H, −8.4E+00, 3.5E−03; TMEM97, −8.3E+00, 5.2E−03; GNL3, −8.3E+00, 1.6E−02; ZC3H15, −8.3E+00, 1.1E−02; DBN1, −8.3E+00, |
| | 1.3E−02; MAP1LC3B, −8.3E+00, 2.9E−04; ID1, −8.3E+00, 1.4E−03; LAPTM4A, −8.2E+00, 4.0E−03; PSAP, −8.2E+00, 3.4E−02; TFRC, −8.2E+00, 1.5E−02; RNF168, |
| | −8.2E+00, 1.3E−02; EIF5, −8.2E+00, 4.0E−02; UBAP2L, −8.2E+00, 3.6E−02; CNN3, −8.2E+00, 3.5E−02; EIF3L, −8.2E+00, 2.8E−02; IMPDH2, −8.2E+00, 7.9E−03; |
| | CBX1, −8.2E+00, 2.4E−02; GHITM, −8.2E+00, 5.9E−04; MRFAP1, −8.2E+00, 9.3E−03; AHCY, −8.1E+00, 6.1E−03; EIF4G1, −8.1E+00, 4.4E−02; TMED10, |
| | −8.1E+00, 8.8E−03; USP1, −8.0E+00, 7.9E−03; EDF1, −8.0E+00, 1.2E−02; DDX21, −8.0E+00, 4.8E−02; ZNF146, −8.0E+00, 4.2E−02; RPN2, −8.0E+00, 2.3E−02; |
| | EID1, −7.9E+00, 2.3E−02; RAB1A, −7.9E+00, 8.6E−03; ATP5PO, −7.9E+00, 2.2E−02; EIF4EBP2, −7.9E+00, 2.6E−02; SLC25A3, −7.9E+00, 3.4E−02; XPO1, |
| | −7.9E+00, 4.3E−02; ATXN7L3B, −7.9E+00, 5.1E−03; ZFAND5, −7.9E+00, 3.7E−02; VAT1, −7.9E+00, 2.6E−03; CDC37, −7.9E+00, 2.4E−03; PSMA7, −7.8E+00, |
| | 4.2E−02; ATP5MF, −7.8E+00, 2.9E−03; CLIC1, −7.7E+00, 2.4E−03; XPOT, −7.7E+00, 4.2E−02; HELLS, −7.7E+00, 3.9E−02; STRAP, −7.7E+00, 2.7E−02; |
| | CDC123, −7.7E+00, 1.7E−02; KARS1, −7.7E+00, 1.2E−02; H2AC8, −7.6E+00, 3.5E−03; CAP1, −7.6E+00, 2.8E−02; SSR3, −7.6E+00, 2.4E−02; PCBP1, −7.6E+00, |
| | 9.1E−03; BSG, −7.6E+00, 4.0E−02; NUP50, −7.6E+00, 1.4E−02; PFDN5, −7.6E+00, 4.8E−03; GPX4, −7.6E+00, 4.9E−03; KHSRP, −7.6E+00, 2.7E−02; CNN2, |
| | −7.5E+00, 2.7E−02; ZNF106, −7.5E+00, 3.9E−02; NDUFS6, −7.5E+00, 7.1E−03; CCND2, −7.5E+00, 4.5E−02; LMAN1, −7.5E+00, 1.4E−02; RPL13AP5, −7.5E+00, |
| | 4.4E−03; VRTN, −7.5E+00, 2.6E−03; MCM7, −7.4E+00, 7.7E−03; ENY2, −7.4E+00, 1.6E−02; PRDX5, −7.4E+00, 6.0E−04; MRPL47, −7.4E+00, 5.2E−04; NHP2, |
| | −7.3E+00, 1.6E−03; ATP5MC3, −7.3E+00, 2.9E−02; RPN1, −7.3E+00, 9.2E−03; HINT1, −7.3E+00, 2.9E−02; SNAR−E, −7.3E+00, 4.4E−03; POLR1D, −7.3E+00, |
| | 1.4E−02; POLR2L, −7.3E+00, 1.2E−03; BAZ2A, −7.3E+00, 3.5E−02; HYOU1, −7.3E+00, 3.7E−02; PRMT1, −7.2E+00, 2.4E−02; DCTN1, −7.2E+00, 7.0E−03; |
| | COX5A, −7.2E+00, 1.1E−02; S100A10, −7.2E+00, 4.4E−04; EIF2AK2, −7.2E+00, 3.4E−02; NIPSNAP1, −7.2E+00, 4.2E−02; ATP5J, −7.1E+00, 6.9E−03; SGO2, |
| | −7.1E+00, 8.4E−03; PGK1, −7.1E+00, 3.3E−02; SNRPD2, −7.1E+00, 4.1E−02; COX7A2, −7.1E+00, 1.7E−02; COX8A, −7.1E+00, 1.3E−02; RDH11, −7.1E+00, 5.1E−03; |
| | RRP1B, −7.1E+00, 2.7E−02; C1QBP, −7.1E+00, 9.5E−03; SF3A3, −7.1E+00, 4.6E−02; FERMT2, −7.0E+00, 3.9E−02; EIF3K, −7.0E+00, 1.8E−02; NDUFA13, |
| | −7.0E+00, 3.4E−02; PPDPF, −7.0E+00, 5.1E−03; ATP6V1G1, −6.9E+00, 1.4E−02; SPARC, −6.9E+00, 1.0E−02; PHB, −6.9E+00, 2.6E−02; PSMD2, −6.9E+00, |
| | 4.2E−02; TCEB2, −6.9E+00, 1.1E−02; RTF1, −6.9E+00, 2.9E−02; PPT1, −6.8E+00, 5.4E−03; HIST1H2AJ, −6.8E+00, 1.4E−02; PRDX2, −6.8E+00, 2.3E−02; |
| | MRPL51, −6.8E+00, 1.3E−02; GMFB, −6.8E+00, 2.3E−02; RANBP1, −6.8E+00, 3.2E−02; COX7B, −6.8E+00, 5.0E−03; SNRPF, −6.7E+00, 2.7E−02; SERP1, |
| | −6.7E+00, 2.6E−02; PCYOX1, −6.7E+00, 8.0E−03; EIF3I, −6.6E+00, 2.3E−02; NME4, −6.6E+00, 7.7E−03; MSN, −6.6E+00, 1.6E−02; SP1, −6.6E+00, 3.7E−02; |
| | WIPI2, −6.6E+00, 4.0E−03; RPL36AL, −6.6E+00, 4.6E−02; SLC25A5, −6.6E+00, 2.7E−02; PLA2G16, −6.5E+00, 3.0E−03; YWHAH, −6.5E+00, 2.3E−02; |
| | MTHFD1, −6.5E+00, 4.6E−02; OSTC, −6.5E+00, 4.5E−02; WDR82, −6.5E+00, 9.4E−03; SNRPE, −6.5E+00, 3.9E−02; EI24, −6.4E+00, 1.7E−03; DKC1, −6.4E+00, |
| | 5.0E−02; C9orf78, −6.4E+00, 5.6E−03; TSPAN6, −6.4E+00, 2.2E−02; TUBB4B, −6.4E+00, 8.0E−03; FKBP4, −6.4E+00, 4.6E−02; NDUFB11, −6.4E+00, 2.8E−03; |
| | RMND5A, −6.4E+00, 4.1E−02; HSPA14, −6.4E+00, 1.2E−02; ARPC5, −6.4E+00, 2.5E−02; CTR9, −6.3E+00, 2.7E−02; PMAIP1, −6.2E+00, 4.4E−02; HIST1H4D, |
| | −6.2E+00, 4.8E−03; RAB13, −6.2E+00, 3.0E−02; SNRPA1, −6.2E+00, 3.3E−02; NSUN2, −6.2E+00, 3.1E−02; NDUFB10, −6.2E+00, 2.0E−02; USP9X, −6.2E+00, |
| | 3.1E−02; PHAX, −6.2E+00, 3.1E−02; NOL7, −6.2E+00, 3.5E−02; MRPL21, −6.1E+00, 1.6E−03; SCOC, −6.1E+00, 2.1E−03; CRABP1, −6.1E+00, 1.7E−02; |
| | AKR1A1, −6.1E+00, 3.7E−02; SNRPB2, −6.1E+00, 2.8E−02; EBNA1BP2, −6.1E+00, 1.3E−02; ZNF680, −6.1E+00, 2.5E−02; SAP18, −6.0E+00, 4.3E−02; COX6C, |
| | −6.0E+00, 2.5E−02; ICMT, −6.0E+00, 4.6E−02; LSM3, −5.9E+00, 3.4E−02; SURF4, −5.9E+00, 1.5E−02; NDUFB8, −5.9E+00, 2.4E−02; NIFK, −5.8E+00, 2.6E−02; |
| | TIMM44, −5.8E+00, 2.1E−03; COA4, −5.8E+00, 4.8E−02; CHP1, −5.7E+00, 3.4E−02; TGIF1, −5.7E+00, 2.5E−02; KDELR1, −5.7E+00, 3.0E−02; HSPE1, |
| | −5.7E+00, 4.6E−02; PSMB6, −5.7E+00, 4.9E−02; UMPS, −5.6E+00, 1.8E−02; UQCRC1, −5.6E+00, 3.3E−02; SNU13, −5.6E+00, 7.9E−03; VIM, −5.6E+00, 4.1E−02; |
| | PSMB3, −5.6E+00, 1.1E−02; LYAR, −5.5E+00, 8.0E−03; TMEM258, −5.5E+00, 1.8E−02; MRPL45, −5.5E+00, 3.9E−03; ZCRB1, −5.5E+00, 2.1E−02; |
| | MYBBP1A, −5.4E+00, 2.7E−03; NOSIP, −5.4E+00, 2.0E−03; PSMD8, −5.4E+00, 4.6E−02; GID8, −5.4E+00, 2.7E−02; NDUFB7, −5.4E+00, 2.3E−02; PSMC4, |
| | −5.3E+00, 7.8E−03; HIST1H3H, −5.3E+00, 2.0E−02; CMAS, −5.3E+00, 3.9E−02; EIF3G, −5.3E+00, 2.8E−02; STOML2, −5.3E+00, 1.7E−02; AKR1B1, −5.3E+00, |
| | 4.8E−02; COPZ1, −5.2E+00, 4.6E−02; PURB, −5.2E+00, 3.6E−02; NGRN, −5.2E+00, 4.8E−02; PGAM1, −5.2E+00, 1.5E−02; HIST1H2AK, −5.2E+00, 4.9E−03; |
| | TMEM261, −5.2E+00, 4.1E−02; NDUFA11, −5.2E+00, 2.5E−02; SNRPG, −5.2E+00, 4.0E−02; PLRG1, −5.2E+00, 4.4E−02; PBX2, −5.1E+00, 2.1E−02; FKBP2, |
| | −5.1E+00, 1.7E−02; METTL5, −5.1E+00, 2.3E−02; AES, −5.1E+00, 1.8E−02; RFC2, −5.1E+00, 3.5E−02; TOMM5, −5.1E+00, 3.6E−02; AURKAIP1, −5.0E+00, |
| | 2.8E−02; NDUFA12, −5.0E+00, 4.1E−02; ARF4, −5.0E+00, 3.6E−02; WDR45B, −5.0E+00, 3.7E−02; SERINC3, −5.0E+00, 2.8E−02; MINOS1, −5.0E+00, 2.6E−02; |
| | CSTB, −4.9E+00, 2.5E−02; C14orf2, −4.9E+00, 4.9E−02; UBQLN2, −4.9E+00, 2.1E−02; ERLEC1, −4.8E+00, 3.0E−03; SSR4, −4.8E+00, 3.6E−02; IFITM3, |
| | −4.8E+00, 3.3E−02; COPS6, −4.8E+00, 3.8E−02; DNAJB1, −4.8E+00, 2.2E−02; POLR2G, −4.8E+00, 1.2E−02; ASAH1, −4.8E+00, 3.4E−02; ARHGD1A, −4.8E+00, |
| | 1.9E−02; HIST4H4, −4.7E+00, 2.1E−02; NDUFA3, −4.7E+00, 4.1E−02; YIPF6, −4.6E+00, 4.4E−02; RAD23A, −4.6E+00, 6.4E−03; EHD4, −4.6E+00, 4.2E−02; |
| | UQCRQ, −4.6E+00, 1.1E−02; DYNLT1, −4.5E+00, 4.1E−02; SPAG7, −4.5E+00, 1.6E−02; SGCB, −4.5E+00, 4.5E−02; DSCC1, −4.4E+00, 1.2E−02; MTERF3, |
| | −4.4E+00, 3.0E−02; LLPH, −4.4E+00, 2.0E−03; ZNHIT1, −4.3E+00, 4.4E−02; RRS1, −4.3E+00, 1.4E−02; PKN1, −4.3E+00, 4.5E−02; THYN1, −4.2E+00, 2.1E−02; |
| | EIF1AY, −4.1E+00, 3.4E−02; SMOC1, −4.0E+00, 1.4E−02; SPP1, −4.0E+00, 8.5E−04; APOPT1, −4.0E+00, 4.2E−02; C8orf33, −3.8E+00, 3.3E−02; HIST2H2BF, |
| | −3.7E+00, 2.7E−02; PEA15, −3.7E+00, 1.3E−02; HSD17B10, −3.7E+00, 4.9E−02; SYT4, −3.6E+00, 3.2E−03; ZNF330, −3.6E+00, 3.5E−02; SIVA1, −3.5E+00, |
| | 3.3E−02; NABP2, −3.5E+00, 2.1E−02; MRPL36, −3.5E+00, 2.3E−02; SURF2, −3.4E+00, 1.1E−02; PRMT6, −3.4E+00, 2.1E−02; WDR18, −3.4E+00, 8.6E−03; |
| | COPS7A, −3.1E+00, 3.1E−02; RMI2, −2.9E+00, 4.1E−02; MAPK3, −2.8E+00, 1.0E−02; TMEM128, −2.8E+00, 3.5E−02; CABLES2, −2.6E+00, 8.6E−03; ELOVL1, |
| | −2.6E+00, 2.9E−02; HHLA1, −2.5E+00, 3.8E−02; KPTN, −2.1E+00, 1.3E−02; ZSCAN31, −2.0E+00, 4.3E−02; PHLDA3, −1.7E+00, 4.6E−02; LOC100996447, |
| | −1.6E+00, 2.5E−02; LOC105372457, −1.6E+00, 4.9E−03; GLI4, −1.5E+00, 4.8E−02; TMEM215, −1.1E+00, 4.6E−02; LOC105373186, −9.8E−01, 1.5E−02; |
| | GHET1, −7.2E−01, 4.0E−02; LOC101929532, −6.0E−01, 2.6E−02; DPPA5, −5.7E−01, 4.5E−02; NXF2B, 2.9E−01, 2.1E−02; RASAL2-AS1, 3.1E−01, 1.4E−02; |
| | SNORD14A, 3.9E−01, 3.7E−02; TMEM88B, 4.1E−01, 2.9E−02; LINC01498, 4.1E−01, 1.6E−02; MIR1267, 4.2E−01, 3.7E−02; LOC102724046, 4.7E−01, 2.6E−02; |
| | LOC102724615, 4.7E−01, 3.4E−02; ZBTB20-AS4, 5.1E−01, 1.6E−02; LOC105374313, 5.3E−01, 1.4E−02; LOC107984704, 5.4E−01, 4.6E−02; |
| | LOC105370647, 5.5E−01, 3.0E−02; LOC105373174, 6.0E−01, 9.7E−03; LOC102723879, 6.4E−01, 3.0E−02; LINC01338, 6.4E−01, 4.8E−02; LOC107984530, |
| | 6.8E−01, 4.6E−02; CCDC129, 6.9E−01, 4.0E−02; FGF16, 8.6E−01, 4.9E−03; LOC105378340, 8.9E−01, 3.3E−02; LOC105377977, 8.9E−01, 1.6E−03; |
| | LOC107984718, 1.0E+00, 2.0E−02; CCDC63, 1.1E+00, 2.3E−02; LOC107986807, 1.2E+00, 1.0E−02; GNB3, 1.3E+00, 4.0E−02; XACT, 1.4E+00, 7.6E−03; |
| | LOC105374970, 1.4E+00, 3.2E−02; LOC105376086, 1.6E+00, 3.0E−02; TMEM108-AS1, 1.6E+00, 2.1E−02; LOC101928731, 1.7E+00, 3.7E−03; |
| | LOC107984492, 1.8E+00, 1.9E−02; LEMD1, 1.8E+00, 2.8E−02; PTH2R, 1.8E+00, 1.6E−02; PLAC4, 2.0E+00, 2.4E−02; LOC105370824, 2.0E+00, 3.1E−02; |
| | LOC100505938, 2.1E+00, 2.7E−02; LOC107986901, 2.1E+00, 4.0E−03; LOC105371657, 2.1E+00, 2.2E−02; NME8, 2.1E+00, 9.1E−04; TBC1D9, 2.2E+00, |
| | 1.4E−02; HNF4G, 2.3E+00, 4.2E−02; STUM, 2.3E+00, 3.4E−03; FAM185A, 2.4E+00, 4.2E−02; LOC101927359, 2.5E+00, 3.0E−03; TRMO, 2.5E+00, 1.2E−02; |
| | HIVEP3, 2.5E+00, 6.9E−03; GPR141, 2.6E+00, 3.2E−02; RNF43, 2.6E+00, 2.6E−02; LOC107985195, 2.6E+00, 8.3E−04; LOC283194, 2.7E+00, 4.0E−02; |
| | PHACTR2-AS1, 2.7E+00, 4.2E−03; ZNF250, 2.7E+00, 3.7E−02; LOC107984754, 2.7E+00, 1.9E−02; XK, 2.8E+00, 2.0E−02; LINC00645, 2.8E+00, 8.4E−03; |
| | LOC100505918, 2.8E+00, 2.7E−02; KCNQ5, 2.8E+00, 4.1E−02; LOC105377130, 2.8E+00, 1.7E−02; LOC105374839, 2.8E+00, 6.9E−03; EBF1, 2.8E+00, |
| | 3.5E−02; LOC105372532, 2.9E+00, 2.6E−03; ABCB5, 2.9E+00, 2.0E−02; LOC107987171, 3.0E+00, 3.1E−02; FBN1, 3.0E+00, 3.6E−02; LOC105370558, |
| | 3.0E+00, 4.8E−02; RRN3P1, 3.0E+00, 3.0E−02; BCL6, 3.1E+00, 2.2E−02; LOC102724861, 3.1E+00, 7.5E−04; FANK1, 3.2E+00, 1.2E−02; LOC105370213, |
| | 3.2E+00, 5.9E−04; SEMA3C, 3.3E+00, 3.2E−02; RGS20, 3.3E+00, 3.8E−02; DDX11-AS1, 3.3E+00, 2.6E−02; OLFM3, 3.3E+00, 1.8E−02; C3orf35, 3.4E+00, |
| | 1.5E−02; LOC105374493, 3.4E+00, 2.6E−02; LOC101927286, 3.4E+00, 6.1E−03; LOC107987081, 3.5E+00, 7.9E−03; KCNK13, 3.5E+00, 1.5E−03; ZIC4, |
| | 3.5E+00, 4.9E−02; NFATC1, 3.5E+00, 3.1E−02; GOLGA8B, 3.5E+00, 9.3E−03; LOC107986293, 3.5E+00, 3.1E−02; PLXNC1, 3.5E+00, 3.3E−02; SUSD1, |
| | 3.6E+00, 2.3E−02; MDFIC, 3.6E+00, 2.1E−02; LINC01339, 3.7E+00, 4.9E−02; NOMO1, 3.7E+00, 2.3E−02; LOC105372633, 3.8E+00, 3.6E−02; PRKCH, |
| | 3.8E+00, 4.2E−02; STPG2-AS1, 3.8E+00, 1.3E−03; PID1, 3.9E+00, 4.3E−02; PPP4R1L, 3.9E+00, 4.4E−02; TNFSF4, 3.9E+00, 7.1E−03; CLEC2A, 3.9E+00, |
| | 6.5E−04; GABRB2, 3.9E+00, 8.0E−03; SLC16A12, 4.0E+00, 1.7E−02; FRMD3, 4.0E+00, 1.7E−02; MIR2052HG, 4.0E+00, 1.5E−02; PTER, 4.1E+00, 1.4E−02; |
| | MBP, 4.1E+00, 3.2E−02; TGFBRAP1, 4.1E+00, 1.6E−02; LPIN1, 4.2E+00, 2.3E−02; TMLHE, 4.2E+00, 2.7E−02; HERC2P3, 4.2E+00, 1.4E−02; PAQR8, |
| | 4.2E+00, 2.8E−02; CELF4, 4.3E+00, 3.1E−02; DENND1B, 4.3E+00, 4.7E−02; ST8SIA6, 4.4E+00, 1.2E−02; ASB18, 4.4E+00, 1.2E−03; NPAS2, 4.4E+00, 6.4E−03; |
| | SERTAD2, 4.4E+00, 4.6E−02; LOC101929077, 4.5E+00, 2.4E−03; C8orf34, 4.5E+00, 4.9E−02; SLC10A7, 4.5E+00, 2.1E−02; PLEKHG1, 4.6E+00, |
| | 1.3E−02; CASC9, 4.6E+00, 1.4E−02; TAPT1-AS1, 4.6E+00, 8.2E−03; TNFAIP8L3, 4.6E+00, 2.3E−02; ARSB, 4.7E+00, 2.5E−02; LDLRAD4, 4.7E+00, 2.9E−02; |
| | LOC107986813, 4.7E+00, 8.0E−04; LOC105373153, 4.7E+00, 1.6E−03; EML5, 4.7E+00, 4.7E−03; LOC105373785, 4.7E+00, 2.8E−02; LOC105371077, |
| | 4.8E+00, 6.2E−03; CACNA1C, 4.8E+00, 4.6E−02; LOC107986022, 4.8E+00, 8.3E−03; FAM73A, 4.8E+00, 2.4E−02; LOC107984294, 4.9E+00, 6.4E−03; |
| | TBC1D22B, 4.9E+00, 3.0E−02; IL1RAPL2, 4.9E+00, 2.6E−02; LOC105377803, 4.9E+00, 7.8E−03; ACVR1B, 5.0E+00, 1.7E−02; ADRA1A, 5.0E+00, 2.6E−03; |
| | NAALAD2, 5.0E+00, 3.9E−02; LINC00937, 5.0E+00, 2.1E−02; PAX7, 5.0E+00, 4.6E−03; MCM9, 5.1E+00, 8.0E−03; B4GALT6, 5.1E+00, 4.2E−02; ANKRD6, |
| | 5.1E+00, 4.1E−02; ME1, 5.1E+00, 1.5E−02; PDGFD, 5.1E+00, 3.3E−02; LM03, 5.1E+00, 9.8E−03; NAPB, 5.2E+00, 9.7E−03; TRHDE, 5.2E+00, 8.2E−03; |
| | LOC101926942, 5.3E+00, 2.7E−03; CASP2, 5.3E+00, 1.1E−02; FAM53B, 5.3E+00, 2.2E−02; TANK, 5.3E+00, 3.7E−02; LINC01057, 5.3E+00, 3.8E−02; |
| | FLRT2, 5.3E+00, 1.1E−02; MCTP2, 5.3E+00, 8.0E−03; ZNF385B, 5.4E+00, 2.6E−02; PDGFC, 5.4E+00, 7.3E−03; KIRREL3, 5.5E+00, 6.3E−04; NKAIN2, |
| | 5.5E+00, 3.7E−02; DHRS7B, 5.5E+00, 4.1E−02; SERGEF, 5.5E+00, 4.2E−02; CAMK4, 5.6E+00, 4.8E−02; LOC105377700, 5.6E+00, 2.0E−02; COBLL1, |
| | 5.6E+00, 9.6E−03; BCAR3, 5.6E+00, 3.8E−02; SCN9A, 5.6E+00, 3.2E−03; LOC107985704, 5.7E+00, 1.8E−02; BMT2, 5.7E+00, 4.6E−02; LOC102724858, |
| | 5.7E+00, 2.8E−07; C9orf85, 5.7E+00, 3.2E−02; LYPD6, 5.7E+00, 3.0E−02; CDH8, 5.7E+00, 2.5E−02; PTCHD1, 5.7E+00, 6.7E−03; VPS50, 5.7E+00, 2.1E−02; |
| | PPARGC1B, 5.8E+00, 3.0E−02; HECW1, 5.8E+00, 4.6E−03; STEAP2, 5.9E+00, 2.4E−02; RORB, 5.9E+00, 4.2E−03; STIM2, 6.0E+00, 6.4E−03; GRB14, |
| | 6.0E+00, 1.3E−02; LRRC1, 6.0E+00, 1.9E−03; CHIC2, 6.0E+00, 2.3E−02; FSTL4, 6.0E+00, 9.2E−03; KHDRBS3, 6.0E+00, 8.2E−03; HCG17, 6.0E+00, 2.4E−02; |
| | EVA1C, 6.0E+00, 1.4E−02; RSRP1, 6.0E+00, 6.4E−03; CEP85L, 6.0E+00, 3.6E−03; LOC105371953, 6.1E+00, 1.5E−02; FKTN, 6.1E+00, 3.1E−03; |
| | ARHGEF26, 6.1E+00, 1.8E−02; USP3, 6.1E+00, 4.0E−03; ZFAT, 6.2E+00, 1.7E−04; RBMS3, 6.2E+00, 1.8E−02; NTN1, 6.2E+00, 3.2E−02; PDE10A, 6.2E+00, |
| | 4.1E−03; TMEM51, 6.2E+00, 3.5E−02; DLEU2, 6.2E+00, 3.1E−02; C1orf101, 6.2E+00, 5.3E−04; RUFY3, 6.2E+00, 4.4E−02; LOC105377959, 6.2E+00, 6.6E−06; |
| | HS6ST2, 6.2E+00, 3.8E−02; SDCCAG8, 6.3E+00, 2.1E−02; CCDC144NL-AS1, 6.3E+00, 1.2E−02; AMOTL1, 6.4E+00, 4.2E−03; FAR2, 6.4E+00, 1.8E−02; |
| | LOC645513, 6.4E+00, 2.0E−02; LOC107987083, 6.4E+00, 4.9E−02; LOC101929563, 6.5E+00, 1.0E−03; FRY, 6.5E+00, 1.5E−02; TSPAN18, 6.5E+00, |
| | 1.1E−02; SLCO5A1, 6.5E+00, 1.1E−03; NRP2, 6.5E+00, 3.7E−03; RPS6KA5, 6.5E+00, 1.1E−02; XRN1, 6.5E+00, 2.9E−02; DGKH, 6.5E+00, 4.7E−02; EPHA4, |
| | 6.5E+00, 4.7E−02; BMP7, 6.5E+00, 1.9E−03; ITGB8, 6.6E+00, 1.9E−03; EMB, 6.6E+00, 2.5E−02; SDC2, 6.6E+00, 3.5E−02; ANKDD1A, 6.6E+00, 3.7E−02; |
| | ABTB2, 6.6E+00, 3.2E−03; GDA, 6.6E+00, 4.8E−03; NEK7, 6.7E+00, 2.3E−02; UMAD1, 6.7E+00, 2.3E−02; FAM193A, 6.7E+00, 4.8E−02; TNRC18, 6.7E+00, |
| | 5.4E−03; VAV2, 6.7E+00, 3.3E−02; C6orf141, 6.7E+00, 3.8E−03; RIC1, 6.7E+00, 2.4E−02; LYPLAL1, 6.7E+00, 4.3E−02; SVIL, 6.8E+00, 1.8E−02; HECW2, |
| | 6.8E+00, 3.6E−03; SLC01A2, 6.8E+00, 7.0E−04; LRP1B, 6.8E+00, 1.9E−03; ZFHX3, 6.8E+00, 1.4E−02; FBXL4, 6.8E+00, 1.2E−02; CDHR3, 6.8E+00, 9.3E−03; |
| | SMAD3, 6.9E+00, 2.3E−02; TDRD3, 6.9E+00, 1.8E−02; IQCB1, 6.9E+00, 1.6E−02; LOC107983984, 6.9E+00, 2.0E−02; UGT8, 6.9E+00, 2.5E−02; |
| | LOC105374455, 6.9E+00, 9.2E−03; ANKH, 7.0E+00, 4.4E−05; AR, 7.0E+00, 1.9E−02; ZNF236, 7.0E+00, 9.2E−03; RNF13, 7.0E+00, 3.4E−02; SMOC2, |
| | 7.0E+00, 1.1E−04; SHROOM2, 7.0E+00, 1.3E−03; FCHSD2, 7.0E+00, 2.0E−02; OPHN1, 7.0E+00, 3.2E−02; PRKY, 7.0E+00, 1.9E−02; BCL11B, 7.1E+00, 3.5E−03; |
| | HTR2C, 7.1E+00, 2.2E−02; MIR124−2HG, 7.1E+00, 9.6E−06; UNC79, 7.1E+00, 1.0E−02; FAM13B, 7.1E+00, 3.4E−02; SLC22A23, 7.1E+00, 4.8E−03; |
| | SCMH1, 7.1E+00, 4.3E−02; FBXL20, 7.1E+00, 6.3E−03; EPN2, 7.1E+00, 3.3E−02; MEMO1, 7.1E+00, 4.7E−02; PPP1R12B, 7.2E+00, 2.5E−02; KANSL1L, |
| | 7.2E+00, 1.6E−02; AGBL4, 7.2E+00, 1.3E−03; LINC01122, 7.2E+00, 2.6E−02; POU6F2, 7.2E+00, 5.2E−03; LRP12, 7.2E+00, 1.7E−03; RAD54B, 7.2E+00, |
| | 4.5E−02; MRPS28, 7.2E+00, 3.5E−02; FGGY, 7.2E+00, 1.5E−02; FAM35A, 7.2E+00, 7.7E−03; JARID2, 7.3E+00, 2.3E−02; RREB1, 7.3E+00, 2.5E−02; TSSC1, |
| | 7.3E+00, 1.5E−02; SDHAP3, 7.3E+00, 1.2E−02; KCNH7, 7.3E+00, 1.1E−03; FAM189A1, 7.4E+00, 2.3E−02; CBFA2T2, 7.4E+00, 3.9E−02; ZNF75D, 7.4E+00, |
| | 2.0E−02; LOC101928437, 7.4E+00, 5.5E−03; RPS6KC1, 7.4E+00, 4.2E−02; REPS1, 7.4E+00, 4.5E−02; UHRF2, 7.4E+00, 1.6E−02; RNF138P1, 7.5E+00, |
| | 7.1E−04; PPHLN1, 7.5E+00, 4.2E−02; FRMD4B, 7.5E+00, 3.6E−04; SNX25, 7.5E+00, 9.5E−03; MEGF10, 7.5E+00, 4.9E−02; DOPEY1, 7.5E+00, 1.2E−02; |
| | MYO1D, 7.5E+00, 4.8E−02; CLCN3, 7.5E+00, 1.4E−02; LOC105369617, 7.5E+00, 2.4E−03; FNIP1, 7.5E+00, 1.9E−02; ETV1, 7.6E+00, 1.4E−02; NRF1, |
| | 7.6E+00, 2.1E−02; SMURF2, 7.6E+00, 2.4E−03; TET3, 7.6E+00, 1.6E−02; LAMA2, 7.6E+00, 5.6E−03; STXBP4, 7.6E+00, 1.0E−02; TBC1D4, 7.6E+00, 2.9E−02; |
| | PDE4D, 7.6E+00, 9.2E−03; EYA1, 7.6E+00, 1.3E−03; MALAT1, 7.6E+00, 2.0E−08; CDK5RAP2, 7.7E+00, 4.4E−02; LOC101928217, 7.7E+00, 1.3E−03; |
| | MIR325HG, 7.7E+00, 4.4E−03; CENPC, 7.7E+00, 4.9E−03; NOVA1, 7.7E+00, 1.0E−02; WNK2, 7.7E+00, 8.4E−03; ANO6, 7.7E+00, 3.3E−02; HCN1, 7.8E+00, |
| | 1.4E−02; CTIF, 7.8E+00, 2.7E−02; NUMB, 7.8E+00, 3.0E−02; SYN3, 7.8E+00, 3.9E−02; GMDS−AS1, 7.8E+00, 8.6E−03; RHBDD1, 7.8E+00, 4.5E−03; IKZF2, |
| | 7.8E+00, 1.0E−03; MON2, 7.8E+00, 1.7E−02; ZNF519, 7.8E+00, 1.8E−02; LOC100506990, 7.8E+00, 1.2E−02; GPR137C, 7.8E+00, 9.8E−03; LPAR1, |
| | 7.9E+00, 1.8E−05; TSHZ3, 7.9E+00, 4.7E−05; GSTCD, 7.9E+00, 1.5E−02; DCBLD2, 7.9E+00, 4.6E−02; CTDSPL, 7.9E+00, 9.2E−03; KATNBL1, 7.9E+00, 7.5E−03; |
| | RTTN, 7.9E+00, 6.4E−03; ANAPC10, 7.9E+00, 3.1E−03; FANCL, 8.0E+00, 3.6E−02; BAZ1A, 8.0E+00, 2.2E−03; ATG7, 8.0E+00, 1.4E−02; ZBTB20, |
| | 8.0E+00, 1.1E−02; NHS, 8.0E+00, 1.1E−02; RIC8B, 8.0E+00, 8.7E−03; LINC00630, 8.0E+00, 6.9E−05; CDK6, 8.0E+00, 3.3E−02; FUT8, 8.0E+00, 1.7E−02; |
| | FTO, 8.1E+00, 4.5E−02; FRMD4A, 8.1E+00, 4.4E−02; MAPK10, 8.1E+00, 8.7E−03; ATP2C1, 8.1E+00, 3.2E−02; C1GALT1, 8.1E+00, 3.3E−02; NELL2, |
| | 8.1E+00, 2.9E−02; LINC00693, 8.1E+00, 1.4E−03; HECTD4, 8.1E+00, 1.2E−02; NNT, 8.2E+00, 8.9E−05; FGF13, 8.2E+00, 8.0E−03; LIMS1, 8.2E+00, |
| | 1.6E−02; KIF21A, 8.2E+00, 3.0E−02; PDE3A, 8.2E+00, 3.5E−02; ZNF407, 8.2E+00, 9.7E−03; LSAMP, 8.2E+00, 4.2E−02; FOXN3, 8.2E+00, 8.9E−04; 4.4E+04, |
| | 8.2E+00, 2.1E−03; CHN1, 8.3E+00, 3.7E−02; PKNOX2, 8.3E+00, 1.2E−04; MAN1A1, 8.3E+00, 1.6E−04; C5orf46, 8.3E+00, 3.4E−03; CEP192, 8.3E+00, |
| | 1.9E−02; C1QTNF3-AMACR, 8.3E+00, 1.5E−03; SEMA5A, 8.3E+00, 5.2E−03; SLX4IP, 8.3E+00, 7.2E−03; KIAA1109, 8.3E+00, 2.8E−02; PHF20L1, 8.3E+00, 7.4E−04; |
| | CPNE8, 8.4E+00, 3.5E−05; DLEU1, 8.4E+00, 1.6E−02; MYO3A, 8.4E+00, 7.7E−04; UBE3D, 8.4E+00, 4.5E−02; ZNF876P, 8.4E+00, 3.5E−05; EPB41L5, |
| | 8.4E+00, 2.5E−02; LRRC16A, 8.4E+00, 4.3E−02; PDZRN3, 8.4E+00, 1.2E−02; TMEM245, 8.4E+00, 1.5E−02; RBM26, 8.4E+00, 4.1E−02; SNX9, 8.4E+00, |
| | 6.8E−03; ACBD6, 8.4E+00, 2.9E−02; ARHGEF9, 8.4E+00, 5.4E−03; LOC105377860, 8.4E+00, 5.7E−03; PIBF1, 8.4E+00, 7.7E−03; MB21D2, 8.5E+00, |
| | 1.0E−02; ERICH1, 8.5E+00, 9.6E−03; AP1S2, 8.5E+00, 4.1E−02; DMXL1, 8.5E+00, 2.9E−02; VWDE, 8.5E+00, 9.0E−03; PELI2, 8.5E+00, 1.6E−02; RBM33, 8.5E+00, |
| | 1.0E−02; ZMIZ1, 8.5E+00, 2.0E−02; KCNIP4, 8.6E+00, 3.1E−03; RICTOR, 8.6E+00, 5.9E−03; STRN3, 8.6E+00, 3.5E−02; DYRK1A, 8.6E+00, 3.7E−02; KIF16B, |
| | 8.6E+00, 8.7E−04; CADPS2, 8.6E+00, 1.6E−02; LDAH, 8.6E+00, 8.0E−03; ATF7IP2, 8.6E+00, 3.1E−03; KCNQ10T1, 8.6E+00, 3.5E−02; LIN7A, 8.6E+00, |
| | 3.5E−03; TMEM131, 8.6E+00, 3.4E−03; CHIC1, 8.6E+00, 1.3E−03; ATP8A1, 8.6E+00, 5.0E−04; PLEKHA1, 8.6E+00, 4.6E−03; PDSS2, 8.7E+00, 1.8E−02; |
| | KIAA1328, 8.7E+00, 2.8E−03; ZHX2, 8.7E+00, 5.2E−03; CDH2, 8.7E+00, 2.9E−03; NSMCE2, 8.7E+00, 2.9E−02; TDRP, 8.7E+00, 2.1E−02; BTBD9, 8.7E+00, |
| | 1.1E−02; ATRX, 8.8E+00, 3.2E−02; MICAL3, 8.8E+00, 8.5E−03; PCNX1, 8.8E+00, 2.3E−02; FGF2, 8.8E+00, 3.0E−02; FNDC3A, 8.8E+00, 2.4E−02; DCAF6, |
| | 8.8E+00, 7.2E−03; SSBP2, 8.8E+00, 3.0E−02; ARHGEF11, 8.8E+00, 4.3E−03; CADM1, 8.9E+00, 9.1E−03; GHR, 8.9E+00, 1.4E−06; TCF7L2, 8.9E+00, 3.8E−03; |
| | HS6ST3, 8.9E+00, 2.4E−03; PTPRZ1, 8.9E+00, 1.9E−02; RAPGEF5, 8.9E+00, 6.8E−03; SPIRE1, 8.9E+00, 5.3E−04; LOC107985675, 8.9E+00, 1.6E−02; |
| | LOC100288637, 8.9E+00, 4.3E−03; DGKI, 8.9E+00, 2.0E−05; BCL11A, 9.0E+00, 2.6E−02; AP3B1, 9.0E+00, 2.5E−02; TRIM44, 9.0E+00, 1.6E−03; CAPRIN2, |
| | 9.0E+00, 1.6E−03; TMEM178B, 9.0E+00, 2.3E−02; DPH6, 9.0E+00, 1.2E−02; FAM172A, 9.0E+00, 1.6E−02; GSE1, 9.0E+00, 2.9E−02; ZGRF1, 9.0E+00, 2.6E−03; |
| | LOC107987166, 9.0E+00, 1.4E−03; LCOR, 9.0E+00, 1.1E−02; CHRM3, 9.0E+00, 1.5E−03; STAG1, 9.1E+00, 2.4E−02; SIK3, 9.1E+00, 2.3E−02; RBPMS, |
| | 9.1E+00, 1.7E−02; NUTM2B-AS1, 9.1E+00, 2.3E−04; LYN, 9.1E+00, 1.6E−04; RFC3, 9.1E+00, 2.3E−02; SLC2A13, 9.1E+00, 1.4E−03; TMTC2, 9.1E+00, 2.0E−02; |
| | KCNMA1, 9.1E+00, 2.7E−03; PPARGC1A, 9.1E+00, 9.7E−03; RALGPS2, 9.1E+00, 9.0E−03; CWC27, 9.1E+00, 1.0E−02; WDR7, 9.1E+00, 7.8E−04; |
| | ARHGAP44, 9.1E+00, 8.7E−04; SBF2, 9.1E+00, 2.2E−02; BRWD3, 9.2E+00, 2.4E−02; MAN2A1, 9.2E+00, 2.2E−02; ST6GALNAC5, 9.2E+00, 1.7E−03; SYNE2, |
| | 9.2E+00, 2.2E−02; CERS6, 9.2E+00, 1.9E−02; SRGAP3, 9.2E+00, 3.9E−04; LOC105377134, 9.2E+00, 2.4E−03; SUCLG2, 9.2E+00, 3.8E−03; ERC1, 9.2E+00, |
| | 2.4E−02; MED12L, 9.2E+00, 3.2E−03; NOS1AP, 9.2E+00, 6.5E−04; FARP1, 9.2E+00, 1.9E−02; POU2F1, 9.2E+00, 2.0E−02; MIPOL1, 9.3E+00, 1.1E−02; |
| | SASH1, 9.3E+00, 1.9E−03; ATP11A, 9.3E+00, 8.5E−03; PHLPP1, 9.3E+00, 1.8E−02; 4.4E+04, 9.3E+00, 1.3E−03; UBE2W, 9.3E+00, 3.2E−03; PARK2, |
| | 9.3E+00, 2.0E−04; CDK13, 9.3E+00, 9.4E−03; ADK, 9.3E+00, 1.8E−02; COA1, 9.3E+00, 1.6E−02; GULP1, 9.3E+00, 1.8E−02; XKR6, 9.4E+00, 1.3E−02; |
| | 4.4E+04, 9.4E+00, 1.2E−02; FBX011, 9.4E+00, 9.6E−03; RUNX1T1, 9.4E+00, 7.6E−03; SCN8A, 9.4E+00, 7.7E−03; GABRB3, 9.4E+00, 1.5E−02; CLASP2, |
| | 9.4E+00, 1.4E−02; NBAS, 9.4E+00, 3.2E−03; WWC1, 9.4E+00, 1.4E−03; TANC2, 9.5E+00, 1.7E−04; COL4A2, 9.5E+00, 1.2E−03; RPS6KA2, 9.5E+00, 7.0E−03; |
| | USP34, 9.5E+00, 1.5E−02; MYO5A, 9.5E+00, 1.2E−03; KCNQ3, 9.5E+00, 1.9E−03; NINL, 9.5E+00, 4.1E−03; SLC22A3, 9.5E+00, 1.4E−05; CMIP, 9.5E+00, |
| | 1.3E−02; EXOC2, 9.5E+00, 1.2E−02; MICU3, 9.5E+00, 1.5E−05; RIMS1, 9.5E+00, 8.2E−05; HIVEP1, 9.6E+00, 8.0E−03; RERE, 9.6E+00, 5.1E−03; PALLD, |
| | 9.6E+00, 2.6E−03; LRP6, 9.6E+00, 9.2E−03; FOXJ3, 9.6E+00, 3.3E−03; DOCK4, 9.6E+00, 2.9E−03; COL4A6, 9.6E+00, 7.9E−05; RNF130, 9.6E+00, 1.1E−02; |
| | EDA, 9.6E+00, 1.5E−02; METTL15, 9.7E+00, 5.0E−03; RNF38, 9.7E+00, 1.4E−02; PIGN, 9.7E+00, 2.0E−04; PGAP1, 9.7E+00, 5.5E−03; LINC-PINT, 9.7E+00, |
| | 1.7E−04; KCNN2, 9.8E+00, 2.0E−03; GPM6A, 9.8E+00, 9.1E−07; GSK3B, 9.8E+00, 8.8E−03; BABAM2, 9.8E+00, 6.0E−04; DISP1, 9.8E+00, 4.7E−04; PDE9A, |
| | 9.8E+00, 7.5E−04; PAK3, 9.8E+00, 1.5E−03; SRGAP1, 9.8E+00, 8.4E−05; RBMS1, 9.8E+00, 9.1E−03; ASAP1, 9.8E+00, 1.6E−02; LCORL, 9.8E+00, 4.2E−03; |
| | MAP7, 9.8E+00, 1.1E−02; CUX1, 9.8E+00, 9.3E−03; NCOA2, 9.8E+00, 1.3E−02; LINC01060, 9.8E+00, 1.0E−04; CNTN4, 9.8E+00, 1.6E−03; DACH2, 9.9E+00, |
| | 2.4E−06; ADAM23, 9.9E+00, 1.1E−03; CHST9, 9.9E+00, 3.3E−03; ADAMTS3, 9.9E+00, 1.5E−06; CNOT2, 9.9E+00, 3.0E−03; TASP1, 9.9E+00, 1.9E−03; |
| | SLC16A2, 9.9E+00, 1.8E−05; UTRN, 1.0E+01, 8.1E−03; SH3RF1, 1.0E+01, 3.5E−04; TNKS, 1.0E+01, 8.8E−03; BAZ2B, 1.0E+01, 1.1E−02; TMTC1, 1.0E+01, |
| | 2.7E−03; PPP2R5E, 1.0E+01, 4.9E−03; COL4A5, 1.0E+01, 4.6E−03; GPM6B, 1.0E+01, 9.2E−03; ZNF292, 1.0E+01, 1.2E−02; STAU2, 1.0E+01, 7.5E−03; |
| | DMTF1, 1.0E+01, 3.0E−03; ERBIN, 1.0E+01, 5.4E−03; PHACTR1, 1.0E+01, 9.6E−03; AFF1, 1.0E+01, 1.1E−02; BBS9, 1.0E+01, 7.7E−03; FAM160A1, |
| | 1.0E+01, 4.8E−03; TBC1D5, 1.0E+01, 8.4E−03; TBC1D32, 1.0E+01, 3.8E−04; LOC105378031, 1.0E+01, 6.0E−04; GPC6, 1.0E+01, 1.7E−02; VIPR2, 1.0E+01, |
| | 3.7E−06; PTPRN2, 1.0E+01, 5.6E−03; CAMK1D, 1.0E+01, 1.6E−03; PTPN14, 1.0E+01, 4.1E−03; ASCC3, 1.0E+01, 7.7E−03; LOC101928096, 1.0E+01, 5.6E−05; |
| | PATJ, 1.0E+01, 6.8E−03; CSRNP3, 1.0E+01, 3.0E−04; FARS2, 1.0E+01, 1.9E−03; KCTD1, 1.0E+01, 8.6E−06; SSH2, 1.0E+01, 1.2E−03; PIAS2, 1.0E+01, |
| | 2.1E−03; SPATA6, 1.0E+01, 5.1E−05; ATAD2B, 1.0E+01, 6.8E−04; LOC102724001, 1.0E+01, 1.5E−04; DST, 1.0E+01, 9.1E−04; FAM13A, 1.0E+01, 9.8E−04; |
| | FAM169A, 1.0E+01, 9.0E−04; SCLT1, 1.0E+01, 1.0E−03; MBOAT2, 1.0E+01, 1.4E−04; AFF2, 1.0E+01, 1.3E−04; SDK2, 1.0E+01, 7.5E−04; BCKDHB, 1.0E+01, |
| | 7.5E−03; IMMP1L, 1.0E+01, 9.0E−05; PTCHD1-AS, 1.0E+01, 7.0E−04; UBAC2, 1.0E+01, 1.5E−03; TEAD1, 1.0E+01, 6.8E−03; KIAA1217, 1.0E+01, 2.8E−03; |
| | UBE3C, 1.0E+01, 2.1E−03; ONECUT1, 1.1E+01, 1.9E−13; LHFPL3, 1.1E+01, 7.9E−08; HIBCH, 1.1E+01, 7.0E−04; NIN, 1.1E+01, 1.0E−04; NEBL, 1.1E+01, |
| | 4.0E−04; ADAMTS6, 1.1E+01, 3.9E−06; PTK2, 1.1E+01, 3.1E−03; CUX2, 1.1E+01, 2.5E−03; SNTG2, 1.1E+01, 1.3E−03; VAV3, 1.1E+01, 3.1E−05; GAB1, |
| | 1.1E+01, 7.6E−05; SLMAP, 1.1E+01, 7.8E−04; MTHFD1L, 1.1E+01, 1.3E−03; CEP170, 1.1E+01, 1.2E−03; SIPA1L1, 1.1E+01, 5.9E−03; SLC24A3, 1.1E+01, |
| | 6.3E−05; ATP9B, 1.1E+01, 4.4E−04; SRPK2, 1.1E+01, 4.6E−03; CRIM1, 1.1E+01, 4.9E−04; JMJD1C, 1.1E+01, 1.5E−04; FSD1L, 1.1E+01, 1.2E−06; WDR70, |
| | 1.1E+01, 6.3E−04; ARHGEF10, 1.1E+01, 3.8E−03; TRMT11, 1.1E+01, 7.5E−04; CDC42BPA, 1.1E+01, 3.0E−03; ROR2, 1.1E+01, 6.0E−05; KALRN, 1.1E+01, |
| | 5.0E−05; STOX2, 1.1E+01, 3.9E−04; MYCBP2, 1.1E+01, 1.7E−03; MTCL1, 1.1E+01, 3.5E−04; ADAMTS12, 1.1E+01, 4.2E−04; STXBP5, 1.1E+01, 8.5E−05; |
| | ST6GAL2, 1.1E+01, 2.5E−05; WDFY2, 1.1E+01, 1.2E−03; PKD2, 1.1E+01, 3.1E−04; LOC107985962, 1.1E+01, 3.5E−05; KLF8, 1.1E+01, 1.5E−03; TSPAN5, |
| | 1.1E+01, 1.0E−04; TULP4, 1.1E+01, 1.8E−03; BARD1, 1.1E+01, 9.4E−05; PLEKHA5, 1.1E+01, 1.3E−04; RNGTT, 1.1E+01, 8.4E−04; KIF13A, 1.1E+01, |
| | 1.6E−03; ZRANB3, 1.1E+01, 8.6E−04; FOCAD, 1.1E+01, 2.0E−03; FBXL17, 1.1E+01, 2.4E−04; RALGAPA2, 1.1E+01, 2.4E−04; AHCYL2, 1.1E+01, 1.2E−03; TTC17, |
| | 1.1E+01, 1.8E−03; SATB2, 1.1E+01, 2.8E−07; PCNX2, 1.1E+01, 2.2E−03; CDYL, 1.1E+01, 1.9E−03; PRICKLE2, 1.1E+01, 9.6E−05; LOC102467213, 1.1E+01, |
| | 4.4E−04; ZC4H2, 1.1E+01, 1.7E−04; AKT3, 1.1E+01, 9.5E−04; CDH6, 1.1E+01, 4.1E−06; MBNL1, 1.1E+01, 2.7E−05; KCTD8, 1.1E+01, 6.5E−05; FRYL, |
| | 1.1E+01, 6.8E−04; CADM2, 1.1E+01, 4.8E−05; MEF2A, 1.2E+01, 6.8E−04; CDC73, 1.2E+01, 1.9E−04; ZFAND3, 1.2E+01, 1.3E−03; KIAA1958, 1.2E+01, |
| | 1.4E−03; PLPP3, 1.2E+01, 8.3E−04; HDAC9, 1.2E+01, 4.6E−05; FAM135A, 1.2E+01, 2.9E−04; DNAH14, 1.2E+01, 2.4E−04; FNDC3B, 1.2E+01, 1.5E−03; GNAQ, |
| | 1.2E+01, 1.3E−03; NAALADL2, 1.2E+01, 7.8E−06; JAKMIP2, 1.2E+01, 4.2E−04; MAML3, 1.2E+01, 1.2E−04; PLCH1, 1.2E+01, 7.4E−04; HIVEP2, 1.2E+01, |
| | 1.9E−06; TMEM131L, 1.2E+01, 1.1E−04; GREB1L, 1.2E+01, 7.9E−05; PARD3B, 1.2E+01, 3.7E−04; DDX10, 1.2E+01, 2.3E−04; MID1, 1.2E+01, 6.9E−04; |
| | SND1, 1.2E+01, 8.5E−04; CCNY, 1.2E+01, 3.9E−04; PVT1, 1.2E+01, 1.3E−03; SUGCT, 1.2E+01, 1.3E−06; FMNL2, 1.2E+01, 1.5E−03; ATXN1, 1.2E+01, 2.6E−04; |
| | NEGR1, 1.2E+01, 5.5E−06; RAP1GDS1, 1.2E+01, 4.4E−05; NECTIN3, 1.2E+01, 4.3E−04; PRIM2, 1.2E+01, 6.9E−04; LINC01572, 1.2E+01, 7.6E−04; |
| | PCSK5, 1.2E+01, 1.9E−05; CBLB, 1.2E+01, 9.0E−05; MLLT10, 1.2E+01, 7.3E−04; CSMD2, 1.2E+01, 8.0E−04; PPP2R3A, 1.2E+01, 9.1E−05; ZNF423, 1.2E+01, |
| | 6.9E−04; MCTP1, 1.2E+01, 1.2E−06; NTM, 1.2E+01, 1.4E−03; FBXW11, 1.2E+01, 5.8E−04; TRABD2B, 1.2E+01, 3.9E−06; LTBP1, 1.2E+01, 1.2E−04; |
| | LOC107986215, 1.2E+01, 8.5E−07; RAPGEF2, 1.2E+01, 3.9E−04; LRRFIP2, 1.2E+01, 7.2E−07; FOXO1, 1.2E+01, 6.0E−04; STK3, 1.2E+01, 2.0E−04; JAZF1, |
| | 1.2E+01, 5.5E−04; RSRC1, 1.2E+01, 1.1E−04; PAM, 1.2E+01, 3.3E−05; NAV3, 1.2E+01, 8.7E−07; MAGI3, 1.2E+01, 4.7E−05; SYT14, 1.2E+01, 3.2E−04; COP1, |
| | 1.2E+01, 2.3E−04; GRHL2, 1.2E+01, 2.9E−04; GLS, 1.2E+01, 9.0E−05; EPHA7, 1.2E+01, 3.2E−07; DPP10, 1.2E+01, 5.2E−04; KANSL1, 1.2E+01, 1.6E−04; |
| | NR3C2, 1.2E+01, 5.7E−08; NEDD4L, 1.2E+01, 4.9E−04; KMT2C, 1.2E+01, 4.4E−04; PRDM5, 1.2E+01, 1.7E−05; ARID2, 1.2E+01, 3.3E−04; TOX3, 1.2E+01, |
| | 7.4E−05; CAMK2D, 1.2E+01, 4.0E−08; ZNF804B, 1.2E+01, 4.9E−10; UBE2E1, 1.3E+01, 1.5E−04; MAP4K3, 1.3E+01, 3.1E−04; TUSC3, 1.3E+01, 1.7E−04; |
| | STK39, 1.3E+01, 8.3E−06; AGPAT4, 1.3E+01, 4.5E−05; PSPC1, 1.3E+01, 1.7E−04; RFX7, 1.3E+01, 3.1E−04; PKN2, 1.3E+01, 1.0E−04; NR5A2, 1.3E+01, |
| | 1.5E−10; CNTN1, 1.3E+01, 5.8E−05; MNAT1, 1.3E+01, 1.0E−04; PUM2, 1.3E+01, 2.0E−04; PRKD1, 1.3E+01, 5.4E−06; MCF2L, 1.3E+01, 9.9E−08; DOCK3, |
| | 1.3E+01, 3.3E−04; MAPK8, 1.3E+01, 1.1E−04; TBL1XR1, 1.3E+01, 9.7E−05; DOK6, 1.3E+01, 8.0E−12; TBC1D22A, 1.3E+01, 4.4E−05; AKAP13, 1.3E+01, |
| | 1.4E−04; C15orf41, 1.3E+01, 7.5E−07; FIGN, 1.3E+01, 9.1E−05; P3H2, 1.3E+01, 3.7E−08; ELP4, 1.3E+01, 4.3E−05; PPP3CA, 1.3E+01, 3.3E−05; |
| | LOC100420587, 1.3E+01, 1.8E−04; RNF220, 1.3E+01, 1.8E−04; KCNT2, 1.3E+01, 1.6E−04; USP25, 1.3E+01, 4.3E−05; TIAM1, 1.3E+01, 1.2E−04; AUH, |
| | 1.3E+01, 1.8E−05; BMPR1A, 1.3E+01, 1.3E−04; PBX3, 1.3E+01, 6.7E−05; LOC285500, 1.3E+01, 2.5E−07; DOCK1, 1.3E+01, 8.8E−05; LINC01515, 1.3E+01, |
| | 4.9E−06; PBX1, 1.3E+01, 1.1E−10; LOC100288798, 1.3E+01, 1.3E−06; PLCB4, 1.3E+01, 4.5E−05; LRRTM4, 1.4E+01, 7.9E−05; TNS3, 1.4E+01, 3.6E−06; |
| | FER, 1.4E+01, 8.0E−06; RAB28, 1.4E+01, 3.3E−06; CHD7, 1.4E+01, 5.8E−05; ASTN2, 1.4E+01, 6.8E−05; IGF2BP2, 1.4E+01, 7.3E−05; LOC107985710, |
| | 1.4E+01, 3.3E−05; CRADD, 1.4E+01, 4.1E−08; COMMD10, 1.4E+01, 6.8E−05; OSBPL10, 1.4E+01, 4.7E−05; ELMO1, 1.4E+01, 2.5E−07; THRB, 1.4E+01, |
| | 2.1E−06; ASIC2, 1.4E+01, 2.9E−06; ITPR2, 1.4E+01, 8.5E−07; CTNND2, 1.4E+01, 4.3E−05; SYT6, 1.4E+01, 4.7E−05; ATRNL1, 1.4E+01, 1.3E−06; ZSWIM6, |
| | 1.4E+01, 4.5E−06; PCDH11X, 1.4E+01, 3.7E−05; KCNG3, 1.4E+01, 5.6E−05; PITPNC1, 1.4E+01, 9.0E−06; ADGRB3, 1.4E+01, 6.2E−07; IL1RAPL1, 1.4E+01, |
| | 1.1E−08; LINC01021, 1.4E+01, 6.9E−07; PAN3, 1.4E+01, 1.9E−05; VTI1A, 1.4E+01, 1.3E−05; EDIL3, 1.4E+01, 1.1E−05; AGAP1, 1.4E+01, 1.6E−05; |
| | SLC24A2, 1.4E+01, 5.4E−06; EXOC6B, 1.4E+01, 2.5E−05; ATXN7L1, 1.4E+01, 1.2E−05; RALYL, 1.4E+01, 9.0E−07; ST6GAL1, 1.4E+01, 3.7E−05; THSD7A, |
| | 1.4E+01, 1.2E−06; LOC105377561, 1.4E+01, 1.8E−14; LUZP2, 1.4E+01, 1.3E−04; B3GALT1, 1.4E+01, 2.0E−09; UNC5C, 1.4E+01, 1.7E−09; PPP1R9A, |
| | 1.5E+01, 1.5E−05; MBD5, 1.5E+01, 1.5E−05; NLGN4Y, 1.5E+01, 1.1E−05; CHD9, 1.5E+01, 1.0E−05; MSI2, 1.5E+01, 5.3E−06; ZNF516, 1.5E+01, 3.6E−09; |
| | SSBP3, 1.5E+01, 8.6E−06; MRTFB, 1.5E+01, 1.5E−06; ULK4, 1.5E+01, 1.9E−06; ALCAM, 1.5E+01, 1.1E−05; MGAT5, 1.5E+01, 1.6E−05; MAP2, 1.5E+01, |
| | 2.1E−06; GTDC1, 1.5E+01, 2.1E−07; FTX, 1.5E+01, 2.0E−08; LRBA, 1.5E+01, 5.3E−06; TMEM135, 1.5E+01, 1.4E−06; PARP8, 1.5E+01, 4.4E−07; GMDS, |
| | 1.5E+01, 7.6E−06; SHROOM3, 1.5E+01, 7.2E−07; FRMD5, 1.5E+01, 1.0E−05; TOX, 1.5E+01, 2.4E−06; NCOA1, 1.5E+01, 5.7E−07; SPATA5, 1.5E+01, 9.5E−06; |
| | LOC107985037, 1.5E+01, 4.7E−08; LOC107986777, 1.5E+01, 2.4E−05; TCF12, 1.5E+01, 4.0E−06; PTPRK, 1.5E+01, 8.6E−06; TMCC1, 1.5E+01, |
| | 1.9E−06; CDH4, 1.5E+01, 1.0E−05; NKAIN3, 1.5E+01, 2.0E−08; PPM1L, 1.5E+01, 6.7E−07; PCCA, 1.5E+01, 5.0E−06; LINC01162, 1.5E+01, 1.4E−06; ZNF827, |
| | 1.5E+01, 7.3E−07; CHST11, 1.5E+01, 8.0E−07; COG5, 1.5E+01, 5.7E−07; IGF1R, 1.5E+01, 5.2E−07; DIP2C, 1.5E+01, 1.3E−06; STK33, 1.5E+01, 2.7E−06; |
| | RAD51B, 1.5E+01, 3.1E−06; TLE4, 1.5E+01, 2.8E−07; SFMBT2, 1.5E+01, 9.5E−09; UTY, 1.6E+01, 1.6E−06; TBL1X, 1.6E+01, 7.4E−07; ST6GALNAC3, |
| | 1.6E+01, 1.7E−06; CDKAL1, 1.6E+01, 1.0E−06; PTPN4, 1.6E+01, 1.4E−06; PTBP2, 1.6E+01, 8.5E−07; ITFG1, 1.6E+01, 1.0E−07; BNC2, 1.6E+01, 1.1E−08; |
| | GALNT17, 1.6E+01, 1.0E−07; NTRK3, 1.6E+01, 3.9E−11; TNIK, 1.6E+01, 2.0E−08; CEP128, 1.6E+01, 8.4E−08; PTPRG, 1.6E+01, 3.6E−13; TMEM108, |
| | 1.6E+01, 3.6E−08; CCDC91, 1.6E+01, 5.3E−08; FGD4, 1.6E+01, 1.8E−08; KDM4C, 1.6E+01, 1.4E−07; FGF12, 1.6E+01, 1.1E−08; TCF4, 1.6E+01, 1.1E−07; |
| | LRIG1, 1.6E+01, 4.6E−08; SEMA6A, 1.6E+01, 3.2E−09; TMEM132B, 1.6E+01, 1.0E−06; PARD3, 1.6E+01, 2.7E−08; ADCY2, 1.6E+01, 8.5E−07; TMEM132D, |
| | 1.6E+01, 6.2E−07; SHISA9, 1.6E+01, 2.4E−07; EXOC4, 1.6E+01, 6.3E−08; ADGRL3, 1.6E+01, 8.7E−09; SDK1, 1.6E+01, 2.1E−07; GALNT7, 1.7E+01, 3.7E−07; |
| | NRXN1, 1.7E+01, 2.7E−09; CTTNBP2, 1.7E+01, 7.6E−08; TRAPPC9, 1.7E+01, 2.9E−09; TBCK, 1.7E+01, 4.1E−09; MAML2, 1.7E+01, 9.1E−08; |
| | LOC101928570, 1.7E+01, 3.2E−10; DPP6, 1.7E+01, 2.2E−08; LOC729732, 1.7E+01, 1.1E−07; BCAS3, 1.7E+01, 2.8E−08; CACNB2, 1.7E+01, 1.9E−08; |
| | ARHGAP42, 1.7E+01, 9.0E−08; BICD1, 1.7E+01, 2.4E−09; WDPCP, 1.7E+01, 7.2E−08; ARHGAP32, 1.7E+01, 4.4E−08; GPHN, 1.7E+01, 7.8E−08; LDLRAD3, |
| | 1.7E+01, 4.6E−08; FRAS1, 1.7E+01, 1.2E−10; FAF1, 1.7E+01, 4.8E−08; PLPP1, 1.7E+01, 1.3E−07; RASAL2, 1.7E+01, 3.6E−08; CACNA2D3, 1.7E+01, |
| | 1.4E−07; ANK2, 1.7E+01, 4.4E−15; TENM2, 1.7E+01, 5.8E−16; SYNDIG1, 1.7E+01, 2.7E−09; VPS13B, 1.8E+01, 4.7E−08; TRPC4, 1.8E+01, 7.8E−10; PSD3, |
| | 1.8E+01, 2.3E−08; ELAVL2, 1.8E+01, 1.2E−13; VWA8, 1.8E+01, 2.3E−10; LGR4, 1.8E+01, 2.2E−08; ARID1B, 1.8E+01, 3.7E−11; SLC35F1, 1.8E+01, 5.8E−10; |
| | GRIN2A, 1.8E+01, 1.3E−12; HS2ST1, 1.8E+01, 1.2E−08; LOC105377862, 1.8E+01, 9.5E−10; EXT1, 1.8E+01, 7.1E−09; CDH9, 1.8E+01, 1.8E−10; ANK3, |
| | 1.8E+01, 1.5E−08; LPP, 1.8E+01, 1.1E−08; DLG2, 1.8E+01, 2.7E−08; ANKRD18CP, 1.8E+01, 9.7E−12; MGAT4C, 1.8E+01, 1.5E−07; ROBO2, 1.8E+01, 2.2E−08; |
| | CAMKMT, 1.8E+01, 5.6E−09; PTPRD, 1.8E+01, 9.2E−09; RIMS2, 1.9E+01, 2.3E−15; SORBS2, 1.9E+01, 4.9E−10; NHSL1, 1.9E+01, 4.6E−09; ERC2, |
| | 1.9E+01, 3.3E−09; RMST, 1.9E+01, 1.3E−09; CHODL, 1.9E+01, 1.1E−09; PLXDC2, 1.9E+01, 3.9E−10; NLGN4X, 1.9E+01, 7.1E−10; FBN2, 1.9E+01, 2.4E−11; |
| | LOC728755, 1.9E+01, 3.5E−09; BBX, 1.9E+01, 2.0E−09; RORA, 1.9E+01, 5.9E−10; SUPT3H, 1.9E+01, 2.9E−10; PLD5, 1.9E+01, 2.3E−11; LARGE, 2.0E+01, |
| | 1.7E−11; MAGI1, 2.0E+01, 1.3E−14; PLCB1, 2.0E+01, 2.4E−10; FOXP1, 2.0E+01, 6.5E−11; TRPS1, 2.0E+01, 9.6E−11; DLGAP1, 2.0E+01, 1.2E−16; MED13L, |
| | 2.0E+01, 5.2E−10; IMMP2L, 2.0E+01, 5.5E−11; LOC102724623, 2.0E+01, 3.0E−14; LOC107986324, 2.0E+01, 4.7E−19; ROBO1, 2.0E+01, 2.2E−10; TENM1, |
| | 2.0E+01, 2.5E−15; GLI3, 2.0E+01, 1.6E−13; LOC644919, 2.0E+01, 1.3E−10; TRIO, 2.1E+01, 6.0E−11; NRXN3, 2.1E+01, 1.9E−10; MACROD2, 2.1E+01, |
| | 1.5E−13; NFIB, 2.1E+01, 3.0E−13; CACNA2D1, 2.1E+01, 5.9E−12; GALNT13, 2.1E+01, 8.6E−13; GRB10, 2.1E+01, 1.9E−12; LINGO2, 2.1E+01, 5.5E−11; UST, |
| | 2.1E+01, 1.3E−11; WWOX, 2.1E+01, 2.5E−13; ARL15, 2.1E+01, 3.8E−12; LDB2, 2.1E+01, 8.4E−12; LINC01098, 2.1E+01, 4.0E−10; XKR4, 2.2E+01, |
| | 1.1E−13; ZNF521, 2.2E+01, 4.2E−14; ADAMTS19, 2.2E+01, 9.6E−13; PRKG1, 2.3E+01, 7.4E−13; CASC15, 2.3E+01, 4.1E−19; SLC1A3, 2.3E+01, 2.2E−16; |
| | FAM155A, 2.3E+01, 2.1E−19; RBFOX1, 2.3E+01, 6.7E−15; TENM3, 2.3E+01, 1.1E−17; TRPM3, 2.3E+01, 6.4E−14; SPIDR, 2.3E+01, 4.1E−14; ROR1, |
| | 2.3E+01, 2.1E−25; KAZN, 2.4E+01, 1.6E−15; PCDH7, 2.4E+01, 3.0E−15; FHIT, 2.4E+01, 8.8E−15; SMYD3, 2.4E+01, 4.6E−15; NBEA, 2.4E+01, 3.6E−16; |
| | GRID2, 2.5E+01, 2.3E−93; RFX3, 2.5E+01, 3.8E−16; RYR2, 2.5E+01, 1.8E−14; SYT1, 2.5E+01, 2.0E−15; ZC3H12B, 2.5E+01, 2.1E−14; HS3ST4, 2.5E+01, |
| | 3.5E−15; LOC107986770, 2.5E+01, 6.1E−15; UBE2E2, 2.5E+01, 5.0E−17; MIR924HG, 2.5E+01, 1.9E−14; TENM4, 2.5E+01, 1.0E−15; LOC101929378, |
| | 2.5E+01, 3.6E−16; SLC25A21, 2.5E+01, 3.9E−16; ADGRV1, 2.5E+01, 1.3E−23; CDK14, 2.6E+01, 1.0E−17; FBXL7, 2.6E+01, 4.4E−19; KLF12, 2.6E+01, 9.9E−18; |
| | GRIP1, 2.6E+01, 5.6E−21; FAT3, 2.6E+01, 2.0E−17; NLGN1, 2.7E+01, 1.9E−18; LOC339862, 2.8E+01, 8.2E−20; KHDRBS2, 2.8E+01, 4.7E−20; MCC, |
| | 2.9E+01, 5.0E−21; SOX5, 2.9E+01, 9.4E−21; NFIA, 3.0E+01, 6.9E−28; NPAS3, 3.1E+01, 7.5E−26; EGFEM1P, 3.1E+01, 1.1E−23; PRKCA, 3.2E+01, 1.4E−24; |
| | SNTG1, 3.3E+01, 2.7E−26; MAGI2, 3.4E+01, 4.8E−32; CTNNA3, 3.8E+01, 1.3E−33; NRG3, 3.9E+01, 8.9E−51; MECOM, 3.9E+01, 2.1E−37 |
| 7-0 | CDH6, −1.3E+01, 4.2E−10; PRTG, −1.2E+01, 8.5E−13; CTNNA2, −9.1E+00, 8.1E−09; GREB1L, −8.9E+00, 3.1E−08; RMST, −8.1E+00, 3.5E−07; DACH1, |
| | −7.5E+00, 1.8E−06; MAPK10, −7.4E+00, 2.7E−06; NRG3, −6.2E+00, 1.9E−04; SOX5, −5.7E+00, 6.3E−03; UNC5C, −5.6E+00, 1.0E−02; TRPM3, −5.6E+00, |
| | 1.7E−03; SEMA6A, −5.5E+00, 2.5E−04; ZNF423, −5.5E+00, 7.2E−04; ADGRL3, −5.4E+00, 7.7E−04; MAP2, −5.2E+00, 3.5E−03; CASC15, −5.2E+00, 2.4E−03; NR6A1, |
| | −5.1E+00, 1.7E−03; ZNF521, −5.0E+00, 2.5E−03; LRRC4C, −5.0E+00, 2.7E−02; PRKG1, −4.8E+00, 3.2E−02; PLEKHA5, −4.7E+00, 6.8E−03; NLGN1, −4.6E+00, |
| | 3.9E−02; PBX1, −4.6E+00, 1.4E−03; PLEKHG4B, −4.4E+00, 4.3E−03; ZFHX4, −4.4E+00, 3.1E−03; TMTC2, −4.1E+00, 4.2E−02; SDK2, −4.1E+00, 2.8E−02; |
| | NRXN1, −4.0E+00, 4.5E−02; EPHA7, −4.0E+00, 9.7E−03; LRP2, −4.0E+00, 1.0E−03; TRIM71, −3.9E+00, 2.9E−02; CRABP1, −3.9E+00, 3.5E−03; PSD3, |
| | −3.8E+00, 4.2E−02; FHOD3, −3.4E+00, 3.3E−02; COL4A6, −3.3E+00, 3.2E−02; FGFBP3, −3.0E+00, 1.7E−02; GRID2, −2.8E+00, 2.0E−04; TMEM132C, −2.6E+00, |
| | 3.0E−02; CCDC160, −2.5E+00, 1.7E−04; SLC16A10, −2.4E+00, 4.5E−02; RIPOR2, −2.2E+00, 2.7E−02; SLC16A2, −2.2E+00, 4.4E−02; MAL2, −2.1E+00, 9.1E−03; |
| | BSX, 1.5E−01, 8.3E−07; COL18A1-AS2, 1.5E−01, 8.3E−07; CTAGE11P, 1.5E−01, 8.3E−07; LOC101928563, 1.5E−01, 8.3E−07; OR5H6, 1.5E−01, 8.3E−07; |
| | FAM90A1, 1.7E−01, 2.0E−03; LOC105374193, 1.8E−01, 3.3E−02; MIR4668, 1.8E−01, 3.3E−02; FOXF2, 1.8E−01, 1.0E−02; LOC105373386, 1.8E−01, |
| | 1.0E−02; LOC105376236, 1.8E−01, 1.0E−02; LOC105378149, 1.8E−01, 1.0E−02; LOC107984772, 1.8E−01, 1.0E−02; LOC107985700, 1.8E−01, 1.0E−02; |
| | LOC107986113, 1.8E−01, 1.0E−02; L3MBTL4-AS1, 1.8E−01, 8.1E−06; LOC102724104, 1.8E−01, 8.1E−06; LOC105371106, 1.8E−01, 8.1E−06; |
| | LOC105372460, 1.8E−01, 8.1E−06; LOC105377022, 1.8E−01, 8.1E−06; MIR130B, 1.8E−01, 8.1E−06; LOC105376603, 2.2E−01, 7.6E−03; LOC107987291, |
| | 2.3E−01, 3.1E−03; LOC105374249, 2.3E−01, 4.7E−03; LOC105371060, 2.4E−01, 4.4E−03; LINC02872, 2.4E−01, 1.8E−03; PFN1P2, 2.4E−01, 2.2E−02; |
| | ERAP2, 2.4E−01, 3.7E−03; GP6, 2.5E−01, 2.4E−04; PPP4R1-AS1, 2.5E−01, 6.6E−04; COX7B2, 2.5E−01, 9.0E−03; LOC101928373, 2.5E−01, 8.4E−04; WDFY4, |
| | 2.5E−01, 1.2E−02; LOC107985352, 2.6E−01, 3.1E−08; LOC107986635, 2.6E−01, 3.1E−08; WT1, 2.6E−01, 3.1E−08; LOC101927801, 2.6E−01, 7.1E−04; |
| | LOC105373372, 2.6E−01, 5.7E−03; LOC107985156, 2.7E−01, 1.0E−02; LINC00692, 2.7E−01, 4.4E−03; CXCL2, 2.7E−01, 2.0E−03; LOC105377367, 2.7E−01, |
| | 2.2E−03; LOC107985950, 2.7E−01, 3.8E−02; CDKN2D, 2.7E−01, 2.4E−02; LOC107986764, 2.8E−01, 2.2E−02; TCAF2, 2.8E−01, 8.3E−03; LOC105371301, |
| | 2.8E−01, 1.7E−03; LOC105371775, 2.8E−01, 1.7E−03; LOC105373863, 2.8E−01, 1.7E−03; MIR548K, 2.8E−01, 1.7E−03; LOC101929450, 2.9E−01, 6.4E−04; |
| | APOD, 3.0E−01, 1.2E−07; KLHL6, 3.0E−01, 1.2E−07; LOC105373186, 3.0E−01, 1.2E−07; LOC105378286, 3.0E−01, 1.2E−07; LOC107986555, 3.0E−01, |
| | 1.2E−07; CCL20, 3.0E−01, 4.1E−02; LOC105373514, 3.1E−01, 4.5E−05; LOC105376072, 3.3E−01, 3.7E−04; LOC101060254, 3.3E−01, 2.1E−02; LOC105371654, |
| | 3.4E−01, 4.1E−03; LOC105373287, 3.4E−01, 4.2E−03; LOC107986077, 3.5E−01, 5.8E−04; LOC105370563, 3.5E−01, 2.0E−02; JSRP1, 3.6E−01, 4.4E−02; |
| | LOC105374596, 3.7E−01, 2.5E−03; LOC105376871, 3.7E−01, 5.0E−03; LOC105377271, 3.7E−01, 1.5E−07; TMCO5B, 3.8E−01, 6.3E−05; LINC02209, 4.1E−01, |
| | 3.3E−03; ARHGD1B, 4.1E−01, 2.4E−02; HMGB4, 4.3E−01, 4.9E−06; OVCH1- S1, 4.3E−01, 1.4E−02; LOC401312, 4.3E−01, 2.6E−02; LOC105377165, 4.3E−01, |
| | 5.6E−03; LRRC32, 4.4E−01, 3.5E−02; PADI1, 4.4E−01, 1.3E−02; LOC407835, 4.6E−01, 8.6E−03; SP100, 5.1E−01, 4.4E−02; LINC00159, 5.1E−01, 2.5E−02; |
| | LOC101929426, 5.4E−01, 7.6E−03; CRYBB2, 5.6E−01, 1.8E−02; ZC2HC1C, 5.8E−01, 2.4E−02; HOXB7, 5.9E−01, 1.7E−02; LOC100506844, 6.5E−01, 1.1E−02; |
| | LOC107986108, 6.6E−01, 8.0E−03; MYOZ2, 6.7E−01, 2.8E−02; LOC100506731, 6.7E−01, 1.3E−03; SNHG9, 7.2E−01, 8.5E−03; LOC107983956, 7.5E−01, |
| | 6.6E−03; LOC101927283, 7.9E−01, 4.8E−03; CFH, 8.1E−01, 4.8E−05; LOC102724210, 8.4E−01, 4.8E−02; SPATA16, 8.5E−01, 1.6E−02; CXCL1, 8.6E−01, |
| | 1.6E−03; LOC100506178, 8.7E−01, 6.9E−03; DCN, 8.9E−01, 8.3E−03; PXDC1, 9.1E−01, 1.8E−03; LOC107986566, 9.2E−01, 1.8E−02; SRPX2, 9.4E−01, 1.3E−04; |
| | GLRA3, 9.4E−01, 1.4E−05; TMEM88, 9.6E−01, 4.9E−02; HOPX, 9.6E−01, 1.6E−08; H19, 9.8E−01, 1.5E−02; TBXT, 1.0E+00, 3.1E−08; DUSP5, 1.0E+00, 3.0E−02; |
| | SFTA1P, 1.0E+00, 1.5E−03; NNMT, 1.0E+00, 2.8E−03; LOC105373997, 1.0E+00, 4.3E−06; WFDC3, 1.0E+00, 5.3E−03; PPP1R3C, 1.1E+00, 5.5E−04; |
| | BMP1, 1.1E+00, 1.7E−02; RUNX1, 1.1E+00, 1.3E−02; NSG1, 1.1E+00, 4.4E−02; C1orf50, 1.2E+00, 1.3E−02; CDKN2B, 1.2E+00, 4.3E−05; BOP1, 1.2E+00, |
| | 8.8E−03; LINC01592, 1.2E+00, 2.4E−02; HAUS7, 1.2E+00, 2.5E−02; MIR503HG, 1.2E+00, 5.0E−05; TNNI1, 1.2E+00, 1.8E−03; PELO, 1.3E+00, 4.6E−02; |
| | NPAS2, 1.3E+00, 4.5E−03; SPX, 1.3E+00, 6.3E−03; ENPEP, 1.3E+00, 2.1E−02; CA3, 1.3E+00, 7.7E−08; RBM24, 1.3E+00, 2.1E−07; EHD2, 1.3E+00, 3.8E−02; |
| | ONECUT2, 1.3E+00, 2.2E−02; BHLHE40, 1.4E+00, 5.6E−03; LOC101927815, 1.4E+00, 1.4E−02; NPPB, 1.4E+00, 5.0E−03; PHLDA2, 1.4E+00, 1.4E−06; |
| | LINC00540, 1.4E+00, 1.5E−02; STAMBPL1, 1.4E+00, 1.5E−02; TLNRD1, 1.4E+00, 5.6E−04; LZTS1, 1.4E+00, 3.3E−02; AKTIP, 1.5E+00, 5.8E−03; TGM2, |
| | 1.5E+00, 2.6E−06; GAS6, 1.5E+00, 2.4E−02; RFK, 1.5E+00, 2.0E−02; NDUFAF3, 1.5E+00, 6.3E−03; PIAS3, 1.5E+00, 3.1E−02; CXCL6, 1.5E+00, 4.6E−07; |
| | LINC00458, 1.5E+00, 1.4E−02; LOC105747689, 1.5E+00, 2.0E−06; DAAM2, 1.6E+00, 2.4E−02; HBEGF, 1.6E+00, 8.0E−04; TM4SF1, 1.6E+00, 5.5E−04; |
| | CMTM3, 1.6E+00, 9.6E−03; JPH2, 1.6E+00, 3.3E−02; MICAL2, 1.6E+00, 2.4E−02; ZNF778, 1.6E+00, 4.2E−02; ATP7B, 1.6E+00, 2.0E−02; ITPRIPL2, |
| | 1.6E+00, 5.5E−04; RNH1, 1.6E+00, 2.4E−02; YPEL2, 1.6E+00, 2.2E−02; SYNM, 1.6E+00, 2.0E−03; GADD45B, 1.7E+00, 5.1E−06; MYRIP, 1.7E+00, 4.2E−02; |
| | MATN3, 1.7E+00, 8.1E−07; LOC101929710, 1.8E+00, 3.3E−02; DYNLT3, 1.8E+00, 2.7E−03; ARHGEF17, 1.8E+00, 4.5E−02; PLXND1, 1.8E+00, 3.2E−02; |
| | NDUFAF8, 1.8E+00, 2.4E−02; TXN2, 1.8E+00, 4.5E−02; EPSTI1, 1.8E+00, 3.4E−04; TGFBI, 1.8E+00, 5.7E−04; GYG1, 1.8E+00, 6.3E−03; EOGT, 1.8E+00, |
| | 1.9E−04; ZBTB2, 1.8E+00, 1.8E−02; CAV2, 1.8E+00, 1.1E−08; KCNH1, 1.9E+00, 5.6E−03; SGK1, 1.9E+00, 7.3E−03; ALAS1, 1.9E+00, 1.3E−02; ASAP3, |
| | 1.9E+00, 4.4E−02; RFTN1, 1.9E+00, 1.8E−03; ELOVL2, 1.9E+00, 7.3E−03; LPL, 1.9E+00, 2.8E−04; ARL2BP, 1.9E+00, 8.8E−03; LOC105377378, 2.0E+00, |
| | 2.3E−06; ODF2, 2.0E+00, 2.7E−02; DAB2, 2.0E+00, 3.8E−02; SERTAD4, 2.0E+00, 2.0E−06; LOC100996643, 2.0E+00, 5.4E−04; LGALS1, 2.0E+00, 2.5E−03; |
| | ZHX3, 2.0E+00, 2.3E−02; RGS3, 2.0E+00, 3.3E−03; CCDC80, 2.0E+00, 2.6E−02; NDUFS2, 2.0E+00, 4.8E−02; NDEL1, 2.0E+00, 1.7E−02; LOC105374007, |
| | 2.0E+00, 9.0E−09; FRG1CP, 2.0E+00, 2.7E−04; POLR2C, 2.0E+00, 4.4E−02; GRHPR, 2.0E+00, 3.6E−02; SLC25A43, 2.0E+00, 4.5E−04; FAM114A1, 2.1E+00, |
| | 1.0E−02; SETD7, 2.1E+00, 1.5E−02; MGLL, 2.1E+00, 1.5E−10; BGN, 2.1E+00, 1.5E−03; GPAT4, 2.1E+00, 4.9E−02; PMP22, 2.1E+00, 7.3E−05; TIPARP, |
| | 2.1E+00, 1.7E−02; TACC1, 2.1E+00, 4.8E−02; PAG1, 2.2E+00, 3.4E−04; PCGF5, 2.2E+00, 4.7E−03; RUSC2, 2.2E+00, 3.6E−02; VGLL3, 2.2E+00, 2.5E−02; |
| | AKAP6, 2.2E+00, 1.7E−03; EFEMP1, 2.2E+00, 3.3E−03; PDGFRB, 2.2E+00, 3.2E−03; RGS20, 2.2E+00, 9.3E−03; FLI1, 2.2E+00, 1.9E−08; CPQ, 2.2E+00, 4.9E−03; |
| | MCAM, 2.3E+00, 5.6E−03; TNFRSF12A, 2.3E+00, 1.0E−02; RIN2, 2.3E+00, 2.1E−03; IFFO2, 2.3E+00, 1.6E−03; CPEB4, 2.3E+00, 2.1E−02; CLMN, |
| | 2.3E+00, 1.4E−03; GYPC, 2.3E+00, 2.5E−05; COBLL1, 2.3E+00, 3.3E−03; ARHGAP29, 2.3E+00, 3.4E−02; PLK2, 2.4E+00, 4.1E−02; AMOTL2, 2.4E+00, 4.8E−02; |
| | DPYD, 2.4E+00, 1.8E−03; DNAJC15, 2.4E+00, 4.9E−02; ZYX, 2.4E+00, 4.2E−02; SBDS, 2.4E+00, 1.9E−02; GSR, 2.4E+00, 2.8E−02; BMP4, 2.5E+00, 9.0E−05; |
| | MSRB3, 2.5E+00, 3.6E−02; IER3, 2.5E+00, 9.6E−06; IQGAP3, 2.5E+00, 5.6E−03; UBASH3B, 2.5E+00, 5.9E−03; COLGALT2, 2.5E+00, 7.3E−04; CMPK1, |
| | 2.5E+00, 1.1E−02; COPS4, 2.6E+00, 3.3E−02; CAVIN1, 2.6E+00, 1.0E−02; TMEM178A, 2.6E+00, 3.8E−04; KCTD16, 2.6E+00, 1.9E−04; SNAPC1, 2.6E+00, |
| | 5.5E−03; RIOK3, 2.6E+00, 3.1E−02; SLC2A1, 2.6E+00, 2.5E−02; AXL, 2.6E+00, 5.2E−03; SYTL2, 2.6E+00, 5.8E−05; DDR2, 2.6E+00, 9.3E−05; BIRC2, |
| | 2.6E+00, 3.2E−02; RNF152, 2.6E+00, 1.8E−02; ZFPM2, 2.6E+00, 4.8E−02; MIR181A1HG, 2.7E+00, 3.2E−02; ANXA6, 2.7E+00, 3.1E−02; CCDC85A, 2.7E+00, |
| | 5.3E−04; MCUR1, 2.7E+00, 1.4E−02; GUCY1A2, 2.7E+00, 1.5E−02; GNG11, 2.7E+00, 8.1E−06; ZC3H18, 2.7E+00, 4.0E−02; GNG12, 2.8E+00, 2.3E−02; |
| | LINC00152, 2.8E+00, 3.7E−04; RBMS2, 2.8E+00, 1.0E−02; CPED1, 2.8E+00, 4.3E−06; CCDC141, 2.8E+00, 1.1E−04; TAGLN2, 2.9E+00, 5.6E−04; ST3GAL1, |
| | 2.9E+00, 8.1E−06; RASA3, 2.9E+00, 5.5E−04; MAP3K20, 2.9E+00, 8.6E−03; CREM, 2.9E+00, 2.8E−02; NOP10, 2.9E+00, 3.1E−02; ADAMTS5, 2.9E+00, |
| | 1.3E−11; MLLT11, 2.9E+00, 1.9E−02; LOC100506718, 2.9E+00, 1.3E−12; VPS29, 2.9E+00, 3.7E−02; FRMD4B, 3.0E+00, 1.4E−02; GALNT10, 3.0E+00, 1.1E−02; |
| | SLC20A1, 3.0E+00, 2.8E−02; ADAMTS1, 3.0E+00, 7.4E−07; RBM20, 3.0E+00, 3.5E−04; CAV1, 3.0E+00, 2.8E−06; LOXL2, 3.0E+00, 1.1E−03; SNX3, 3.0E+00, |
| | 1.0E−03; AFAP1, 3.1E+00, 1.2E−02; MEF2C, 3.1E+00, 3.7E−03; GOLGA3, 3.1E+00, 2.3E−02; EEA1, 3.1E+00, 4.1E−02; TXNDC17, 3.1E+00, 3.7E−02; DPP6, |
| | 3.1E+00, 4.8E−02; SEC23A, 3.1E+00, 1.5E−02; ARID5B, 3.2E+00, 2.0E−03; SAMD5, 3.2E+00, 4.2E−04; COL12A1, 3.2E+00, 6.2E−05; PHTF2, 3.2E+00, 2.0E−02; |
| | ARSJ, 3.2E+00, 3.5E−07; MARCHF1, 3.2E+00, 4.6E−02; ARF4, 3.2E+00, 1.8E−02; NRK, 3.2E+00, 7.0E−08; NECTIN2, 3.2E+00, 2.8E−02; PSMA6, 3.2E+00, |
| | 3.9E−02; PCBP1, 3.3E+00, 1.7E−02; MCM10, 3.3E+00, 2.7E−02; PRR11, 3.3E+00, 3.8E−02; PALM2AKAP2, 3.3E+00, 7.7E−05; LINC-PINT, 3.3E+00, 3.3E−03; |
| | CNTN6, 3.3E+00, 2.1E−03; CDC123, 3.4E+00, 4.2E−02; MYL12A, 3.4E+00, 4.5E−02; SEC24D, 3.4E+00, 3.0E−03; ARHGAP10, 3.4E+00, 1.1E−02; |
| | ADGRG6, 3.4E+00, 2.5E−06; PDGFC, 3.4E+00, 1.8E−03; TTTY15, 3.4E+00, 1.9E−04; EDIL3, 3.4E+00, 3.5E−02; DOCK9, 3.5E+00, 1.7E−03; TUBA1A, |
| | 3.5E+00, 3.8E−02; RSU1, 3.5E+00, 3.1E−02; PRKAG2, 3.5E+00, 1.1E−04; COL5A1, 3.5E+00, 3.3E−04; EDN1, 3.5E+00, 1.4E−06; TMTC1, 3.5E+00, 2.4E−02; |
| | CDC42, 3.5E+00, 4.4E−02; LOC151760, 3.6E+00, 6.4E−05; WDR1, 3.6E+00, 3.3E−02; KLF6, 3.6E+00, 5.9E−05; RUNX2, 3.6E+00, 2.1E−09; TENM2, 3.6E+00, |
| | 3.9E−02; NUAK1, 3.6E+00, 1.7E−04; EIF4G2, 3.6E+00, 4.9E−02; MALAT1, 3.6E+00, 3.2E−07; STARD13, 3.6E+00, 4.1E−07; PCNX4, 3.7E+00, 2.0E−02; |
| | CLSTN2, 3.7E+00, 3.2E−06; P3H2, 3.7E+00, 3.5E−02; CYCS, 3.7E+00, 3.7E−02; KAT6A, 3.7E+00, 2.5E−02; ACTR3, 3.7E+00, 4.8E−02; SPATS2L, 3.7E+00, |
| | 3.6E−02; TFPI2, 3.7E+00, 1.4E−08; CSRP1, 3.7E+00, 7.0E−06; LUM, 3.7E+00, 2.7E−08; KRT8, 3.7E+00, 2.0E−02; CAST, 3.8E+00, 4.7E−04; CEP170, 3.8E+00, |
| | 4.6E−02; SGIP1, 3.8E+00, 4.4E−09; MYL9, 3.8E+00, 7.4E−03; MYL12B, 3.8E+00, 1.9E−03; SWAP70, 3.8E+00, 8.3E−03; PRKCA, 3.8E+00, 3.6E−02; |
| | MARCHF4, 3.8E+00, 3.8E−09; RHOA, 3.9E+00, 3.5E−02; MAMDC2, 3.9E+00, 5.5E−03; GNB4, 3.9E+00, 3.7E−04; ELK3, 3.9E+00, 3.4E−05; PRDX6, 3.9E+00, |
| | 2.9E−02; RAB6A, 3.9E+00, 1.1E−02; FHL1, 3.9E+00, 1.7E−05; CAP1, 3.9E+00, 2.8E−02; LDHA, 3.9E+00, 7.5E−03; MBNL2, 3.9E+00, 4.7E−05; RAB31, |
| | 4.0E+00, 1.4E−04; ATXN1, 4.0E+00, 3.7E−03; C2CD3, 4.0E+00, 1.4E−03; IDI1, 4.0E+00, 7.9E−03; OGFRL1, 4.0E+00, 3.1E−08; PLS3, 4.0E+00, 4.3E−02; |
| | ETS1, 4.0E+00, 7.4E−06; PPP6R3, 4.0E+00, 3.6E−02; TJP1, 4.0E+00, 3.8E−02; RHOBTB3, 4.0E+00, 1.9E−05; MYH10, 4.1E+00, 1.7E−02; NEXN, 4.1E+00, |
| | 3.1E−04; TPM2, 4.1E+00, 2.6E−02; TNFRSF21, 4.1E+00, 4.1E−03; ANTXR1, 4.1E+00, 8.4E−03; SKP1, 4.1E+00, 4.6E−02; PTPRM, 4.1E+00, 4.8E−02; UGCG, |
| | 4.1E+00, 5.9E−06; IGFBP3, 4.1E+00, 1.6E−07; S100A10, 4.2E+00, 1.9E−04; KIF5B, 4.2E+00, 1.5E−02; CNN3, 4.2E+00, 2.6E−02; FAM126A, 4.2E+00, 6.0E−03; |
| | TMEM123, 4.2E+00, 1.4E−03; ILF2, 4.2E+00, 1.6E−02; RNF217, 4.2E+00, 1.7E−03; TRAM2, 4.2E+00, 4.4E−05; ADGRL4, 4.2E+00, 5.5E−12; PPFIBP1, |
| | 4.3E+00, 2.4E−03; ROCK2, 4.3E+00, 8.7E−03; DUSP6, 4.3E+00, 1.9E−05; SYNE1, 4.3E+00, 1.3E−08; HIF1A, 4.3E+00, 1.2E−02; FLNC, 4.3E+00, 2.8E−04; |
| | CALR, 4.3E+00, 1.8E−02; ACAT2, 4.4E+00, 9.0E−03; NTM, 4.4E+00, 2.9E−02; USP53, 4.4E+00, 2.8E−05; LAMB1, 4.4E+00, 7.5E−03; HSPA5, 4.4E+00, 9.3E−03; |
| | ARPC2, 4.4E+00, 8.8E−03; CDH13, 4.5E+00, 4.0E−05; DNMT1, 4.5E+00, 4.2E−03; RAI14, 4.5E+00, 1.2E−02; PFKP, 4.5E+00, 9.2E−05; EIF4G1, 4.6E+00, |
| | 6.7E−03; SYTL5, 4.6E+00, 1.9E−09; MBNL1, 4.6E+00, 2.0E−04; TANC1, 4.6E+00, 9.3E−05; HOOK3, 4.7E+00, 9.7E−05; PLXNA2, 4.7E+00, 7.4E−10; OSBPL8, |
| | 4.7E+00, 4.7E−03; TFPI, 4.7E+00, 8.2E−06; MAP4, 4.7E+00, 5.5E−03; HMCN1, 4.7E+00, 2.1E−04; NQO1, 4.7E+00, 5.6E−07; MSN, 4.8E+00, 5.9E−04; CALU, |
| | 4.8E+00, 4.7E−03; DSTN, 4.8E+00, 6.5E−03; NT5C2, 4.8E+00, 2.3E−03; ADAMTS12, 4.8E+00, 7.3E−03; CCT6A, 4.8E+00, 4.5E−03; SPTBN1, 4.8E+00, 5.0E−03; |
| | DIAPH3, 4.9E+00, 1.3E−02; TUBB6, 4.9E+00, 1.3E−06; SH3BP4, 4.9E+00, 3.9E−05; CLIC1, 4.9E+00, 2.4E−04; LAMC1, 5.0E+00, 2.0E−04; SSR3, 5.0E+00, |
| | 2.9E−04; TES, 5.0E+00, 2.8E−10; PRSS23, 5.0E+00, 1.2E−07; NEK7, 5.0E+00, 1.0E−05; ATRNL1, 5.0E+00, 2.0E−04; ELL2, 5.0E+00, 9.5E−06; TLN1, |
| | 5.0E+00, 1.2E−05; KTN1, 5.1E+00, 1.3E−03; COPG2, 5.1E+00, 1.9E−03; SHISA9, 5.1E+00, 7.6E−03; COL1A2, 5.1E+00, 1.2E−03; CCND1, 5.1E+00, 1.9E−03; |
| | YWHAZ, 5.2E+00, 1.4E−03; INPP4B, 5.2E+00, 3.3E−05; MAP1B, 5.2E+00, 1.1E−03; CCND2, 5.2E+00, 2.0E−03; ALPK2, 5.3E+00, 9.5E−14; LIMS1, 5.4E+00, |
| | 1.3E−04; PFN1, 5.4E+00, 2.8E−03; FLNA, 5.4E+00, 1.0E−03; CLIC4, 5.4E+00, 4.5E−04; FTL, 5.5E+00, 4.8E−04; PARVA, 5.5E+00, 9.3E−08; RIC1, 5.5E+00, |
| | 6.7E−06; CAPN2, 5.5E+00, 5.0E−09; ACTB, 5.5E+00, 8.3E−07; NEDD9, 5.6E+00, 1.4E−06; CAP2, 5.6E+00, 1.2E−06; TUBA1B, 5.6E+00, 5.5E−04; CORO1C, |
| | 5.6E+00, 7.8E−06; COL3A1, 5.7E+00, 5.6E−13; S100A11, 5.8E+00, 3.3E−07; ITGA1, 5.8E+00, 4.8E−10; MYOF, 5.8E+00, 8.8E−09; SERPINE1, 5.9E+00, 4.0E−12; |
| | COL5A2, 5.9E+00, 8.3E−07; CDK6, 5.9E+00, 3.1E−05; ZEB2, 5.9E+00, 4.7E−05; NEAT1, 6.0E+00, 6.2E−04; ACTA2, 6.1E+00, 1.3E−10; MYL6, 6.1E+00, |
| | 1.2E−04; ACTC1, 6.2E+00, 2.7E−07; NES, 6.2E+00, 1.1E−04; FLNB, 6.4E+00, 1.1E−05; TMSB10, 6.5E+00, 1.8E−04; TRIO, 6.5E+00, 7.7E−05; GLS, 6.6E+00, |
| | 2.1E−07; ACTG1, 6.7E+00, 9.7E−07; FSTL1, 6.7E+00, 3.8E−06; DKK2, 6.8E+00, 7.4E−18; ADAM19, 6.8E+00, 3.5E−12; SERPINE2, 6.8E+00, 6.7E−12; MACF1, |
| | 6.9E+00, 7.8E−08; MEST, 7.1E+00, 1.2E−07; ACTN4, 7.1E+00, 1.4E−07; SMURF2, 7.1E+00, 4.4E−12; SORBS2, 7.1E+00, 7.8E−08; PALLD, 7.2E+00, 6.4E−06; |
| | SAMD4A, 7.4E+00, 5.1E−06; SPARC, 7.5E+00, 9.0E−09; DLC1, 7.5E+00, 5.1E−20; LPP, 7.6E+00, 7.6E−07; SPP1, 7.6E+00, 4.6E−12; FRMD6, 7.7E+00, |
| | 7.1E−16; PPME1, 8.0E+00, 4.9E−13; COL1A1, 8.0E+00, 1.8E−11; TPM4, 8.1E+00, 7.7E−09; TGFB2, 8.4E+00, 2.6E−22; PICALM, 8.6E+00, 1.5E−10; TMSB4X, |
| | 9.0E+00, 7.7E−09; ACTN1, 9.2E+00, 7.4E−11; EXT1, 9.3E+00, 2.2E−09; COL4A2, 9.4E+00, 9.2E−16; THBS1, 9.6E+00, 8.4E−16; ITGB1, 9.6E+00, 4.4E−12; |
| | IGFBP7, 9.8E+00, 1.8E−35; MIR4435-2HG, 1.0E+01, 5.1E−17; MYH9, 1.0E+01, 1.2E−13; RBMS3, 1.1E+01, 8.9E−13; COL4A1, 1.1E+01, 8.5E−22; VIM, |
| | 1.1E+01, 2.2E−15; ANXA2, 1.2E+01, 9.8E−19; SEPTIN11, 1.2E+01, 8.8E−18; ITGAV, 1.2E+01, 3.1E−17; AHNAK, 1.2E+01, 2.0E−17; AKAP12, 1.2E+01, 3.4E−17; |
| | CRIM1, 1.2E+01, 1.1E−18; TPM1, 1.3E+01, 8.5E−22; ANXA1, 1.3E+01, 1.2E−27; COL8A1, 1.3E+01, 2.0E−50; CYR61, 1.3E+01, 1.1E−32; TAGLN, |
| | 1.4E+01, 4.7E−22; FLRT2, 1.4E+01, 6.8E−29; FN1, 1.4E+01, 5.3E−19; MAML2, 1.4E+01, 2.9E−23; DDAH1, 1.5E+01, 4.2E−31; CALD1, 1.6E+01, 1.5E−28; |
| | CCN2, 1.6E+01, 2.8E−38; ANKRD1, 2.9E+01, 1.2E−62 |
| 7-1 | H2AC17, −2.3E+00, 5.0E−04; H1-2, −2.2E+00, 9.1E−04; TPM1, −1.9E+00, 6.8E−04; H2AC20, −1.8E+00, 1.1E−02; H4C3, −1.7E+00, 3.1E−02; H1-5, −1.7E+00, |
| | 2.7E−02; DYNLL1, −1.7E+00, 2.7E−03; FN1, −1.6E+00, 3.4E−03; SRRM1, −1.6E+00, 1.2E−02; H2AC12, −1.5E+00, 1.8E−02; SUPT16H, −1.5E+00, 2.7E−02; |
| | SET, −1.4E+00, 2.0E−02; NDUFS5, −1.4E+00, 2.5E−02; EPCAM, −1.4E+00, 2.5E−02; NOLC1, −1.3E+00, 3.2E−02; HSPD1, −1.3E+00, 2.8E−02; MEST, −1.3E+00, |
| | 2.9E−02; H4C12, −1.3E+00, 3.4E−02; H3C10, −1.3E+00, 3.6E−02; WBP11, −1.2E+00, 7.4E−03; RPL11, −1.2E+00, 3.0E−02; ID2, −1.2E+00, 7.0E−04; IARS2, |
| | −1.1E+00, 2.0E−02; PTMA, −1.1E+00, 1.0E−02; HSP90AA1, −1.0E+00, 3.5E−02; H2BC10, −1.0E+00, 2.0E−02; BANF1, −1.0E+00, 2.9E−02; CA11, −8.4E−01, |
| | 1.8E−02; UNC5C, −7.5E−01, 3.1E−02; ID4, −6.9E−01, 4.2E−02; RPP38, −6.0E−01, 3.0E−02; RIMS4, −5.1E−01, 2.9E−02; LOC105377207, 1.8E−01, 4.7E−02; |
| | BCL6B, 2.5E−01, 4.6E−02; LOC107985892, 2.6E−01, 4.8E−02; ECSCR, 2.6E−01, 4.6E−03; DIPK2B, 3.1E−01, 3.6E−02; SULT1C3, 3.2E−01, 5.3E−03; THSD1, |
| | 3.3E−01, 1.7E−02; LINC01594, 3.3E−01, 4.7E−03; PCDH12, 3.4E−01, 5.0E−03; SH2D3C, 3.5E−01, 3.8E−02; PTPRE, 3.5E−01, 1.6E−02; HHEX, 3.6E−01, 3.6E−03; |
| | FLT4, 3.9E−01, 1.7E−02; ST8SIA4, 4.2E−01, 2.0E−02; PTPRB, 4.5E−01, 1.0E−02; TMEM255B, 4.5E−01, 3.5E−03; ESAM, 4.7E−01, 9.5E−05; PCED1B, 4.7E−01, |
| | 8.4E−03; MMRN1, 4.8E−01, 1.0E−05; TAL1, 4.9E−01, 6.7E−03; HLA-E, 5.1E−01, 1.0E−02; SULT1C2, 5.1E−01, 1.8E−02; KCNA6, 5.1E−01, 4.2E−03; PMP22, |
| | 5.2E−01, 2.5E−02; SGIP1, 5.4E−01, 1.5E−02; ENG, 5.9E−01, 1.5E−02; JCAD, 6.1E−01, 2.3E−02; AFAP1L1, 6.1E−01, 2.0E−03; LPAR6, 6.2E−01, 2.3E−02; |
| | MALAT1, 6.6E−01, 1.6E−02; PECAM1, 6.7E−01, 3.3E−05; SLC22A23, 6.8E−01, 2.2E−02; RELL1, 6.9E−01, 2.0E−02; RAMP2, 6.9E−01, 2.5E−03; PAG1, 7.1E−01, |
| | 4.7E−02; LOC105369309, 7.2E−01, 7.6E−05; SULT1C2P1, 7.3E−01, 4.6E−07; F2RL2, 7.3E−01, 2.1E−04; PLXND1, 7.4E−01, 6.7E−03; ALDH1A2, 7.5E−01, |
| | 2.2E−03; GUCY1A1, 7.5E−01, 4.5E−04; DYRK4, 7.6E−01, 3.4E−04; ITGA9, 7.7E−01, 1.8E−02; RAB3C, 8.0E−01, 4.6E−03; ERG, 8.0E−01, 9.1E−06; TIE1, 8.0E−01, |
| | 1.0E−06; FGD5, 8.2E−01, 3.4E−04; TGFBR2, 8.2E−01, 2.5E−02; CDH5, 8.3E−01, 7.0E−08; DOCK6, 8.3E−01, 3.0E−02; SYNE1, 8.3E−01, 4.5E−03; CNIH3, |
| | 8.4E−01, 1.8E−02; COL15A1, 8.5E−01, 2.1E−05; EGFL7, 8.7E−01, 9.4E−04; ARHGAP31, 8.9E−01, 3.6E−04; S1PR3, 9.1E−01, 2.6E−03; IQSEC1, 9.5E−01, 2.8E−02; |
| | ASXL1, 9.6E−01, 2.3E−02; NETO1, 9.9E−01, 3.4E−02; FAM189A1, 9.9E−01, 4.8E−02; FAM107B, 9.9E−01, 2.0E−02; GRIN2A, 1.0E+00, 4.6E−03; SHOC1, |
| | 1.0E+00, 3.2E−02; PLVAP, 1.0E+00, 9.8E−07; FAR2, 1.0E+00, 3.5E−02; MTSS1, 1.0E+00, 1.2E−03; BMP2K, 1.0E+00, 3.6E−02; CALCRL, 1.0E+00, 1.0E−06; |
| | DAB2, 1.1E+00, 3.9E−03; DYSF, 1.1E+00, 8.3E−06; ANO3, 1.1E+00, 8.0E−03; CDK17, 1.1E+00, 3.1E−02; RHOJ, 1.1E+00, 5.3E−08; RASGRP3, 1.1E+00, 6.2E−09; |
| | ATP8B4, 1.1E+00, 2.5E−04; PLEKHG1, 1.1E+00, 1.0E−06; GREM2, 1.1E+00, 9.3E−03; STARD13, 1.1E+00, 3.2E−03; DACH2, 1.1E+00, 4.8E−02; CD34, |
| | 1.2E+00, 6.2E−09; EMCN, 1.2E+00, 6.2E−09; CHRM3, 1.2E+00, 5.2E−04; SAMD5, 1.2E+00, 2.7E−02; PLXNA2, 1.3E+00, 2.2E−02; ETS1, 1.3E+00, 4.1E−04; |
| | KIF26B, 1.3E+00, 6.8E−03; MAST4, 1.3E+00, 2.2E−04; TFPI, 1.3E+00, 2.7E−02; NRP2, 1.4E+00, 1.4E−03; NHSL2, 1.4E+00, 1.0E−02; COL5A2, 1.4E+00, |
| | 2.3E−02; PRSS23, 1.4E+00, 3.3E−05; COL4A1, 1.4E+00, 1.3E−02; KDR, 1.4E+00, 9.7E−04; CPED1, 1.4E+00, 6.0E−03; GAB1, 1.5E+00, 9.7E−04; CACNA1C, |
| | 1.5E+00, 3.9E−04; ELK3, 1.5E+00, 2.7E−05; INPP4B, 1.5E+00, 2.7E−02; CALD1, 1.5E+00, 2.0E−02; ITPR1, 1.5E+00, 1.1E−03; FLT1, 1.5E+00, 5.7E−03; |
| | MEF2A, 1.5E+00, 1.2E−02; SSBP2, 1.6E+00, 2.0E−02; EBF1, 1.6E+00, 1.2E−03; LDB2, 1.6E+00, 9.9E−03; SESN3, 1.7E+00, 2.7E−02; DLC1, 1.7E+00, 7.7E−03; |
| | ARL15, 1.7E+00, 2.5E−03; CDC42BPA, 1.7E+00, 2.4E−03; ZNF521, 1.7E+00, 9.7E−04; MYO6, 1.7E+00, 1.5E−04; AKT3, 1.8E+00, 1.7E−03; COL3A1, |
| | 1.8E+00, 1.8E−02; LIMCH1, 1.9E+00, 5.8E−05; TCF4, 2.0E+00, 5.2E−04; ARHGAP28, 2.0E+00, 5.9E−05; PCDH17, 2.1E+00, 5.0E−07; COL4A2, 2.1E+00, |
| | 2.2E−07; CDH13, 2.1E+00, 6.0E−09; ITGAV, 2.1E+00, 6.8E−04; ZFPM2, 2.1E+00, 2.7E−09; PTCHD4, 2.2E+00, 9.1E−06; FLRT2, 2.2E+00, 2.7E−05; PTPRM, |
| | 2.3E+00, 2.4E−05; FBN1, 2.4E+00, 4.1E−07; VAV3, 2.4E+00, 6.9E−10; GNG11, 2.4E+00, 2.0E−13; HAPLN1, 2.6E+00, 2.6E−09; ZEB1, 2.6E+00, 7.2E−11; |
| | SAMD4A, 2.6E+00, 1.8E−06; DOCK4, 2.6E+00, 1.5E−07; ROBO2, 2.7E+00, 4.0E−05; ADAM12, 3.2E+00, 4.0E−17; FLI1, 3.4E+00, 2.8E−22; KALRN, 3.7E+00, |
| | 4.0E−17; ADGRL4, 4.2E+00, 6.2E−27; MEF2C, 4.3E+00, 3.4E−27 |
| 7-2 | ATG16L2, 3.3E+00, 6.5E−03; MEF2C, 7.7E+00, 1.2E−02 |
| 8-0 | HSPA8, −3.1E+01, 1.1E−03; RPS3, −3.0E+01, 2.4E−02; NPM1, −3.0E+01, 9.4E−05; SNRPF, −2.5E+01, 3.5E−02; RPLP1, −2.4E+01, 2.3E−02; C4orf51, −1.9E+01, |
| | 3.3E−02; EEF1A1, −1.9E+01, 3.6E−02; PTMA, −1.7E+01, 1.1E−02; C19orf67, 1.0E+00, 3.5E−02; GASK1B, 1.0E+00, 3.5E−02; LINC01565, 1.0E+00, 3.5E−02; |
| | LOC101929544, 1.0E+00, 3.5E−02; LOC105373289, 1.0E+00, 3.5E−02; LOC105377508, 1.0E+00, 3.5E−02; LOC107984980, 1.0E+00, 3.5E−02; |
| | LOC107986561, 1.0E+00, 3.5E−02; PABPC1P2, 1.0E+00, 3.5E−02; RGMB-AS1, 1.7E+00, 3.1E−02; LOC101928907, 1.7E+00, 3.5E−02; LOC105379508, |
| | 1.7E+00, 3.5E−02; RNF112, 1.7E+00, 7.4E−03; LOC107985448, 1.9E+00, 3.5E−02; IL12A, 1.9E+00, 3.2E−02; MIR4419A, 2.2E+00, 3.3E−02; |
| | LOC101927281, 2.3E+00, 3.9E−04; LOC105372592, 2.3E+00, 3.9E−04; LOC151484, 2.3E+00, 3.9E−04; SNORD57, 2.3E+00, 3.5E−02; LOC101928535, |
| | 2.9E+00, 4.6E−03; ANKRD20A9P, 2.9E+00, 2.3E−03; LOC105374459, 3.1E+00, 3.3E−02; SOCS2-AS1, 3.1E+00, 3.3E−02; LOC102724419, 3.2E+00, 5.0E−02; |
| | LOC105374723, 3.5E+00, 3.2E−02; LOC105370981, 3.8E+00, 6.4E−04; LOC107986807, 4.0E+00, 8.4E−04; TIGD6, 4.1E+00, 3.1E−04; RUNX2, |
| | 4.2E+00, 5.0E−02; KLHL17, 4.4E+00, 3.2E−02; BBS5, 5.0E+00, 3.4E−05; LOC102723465, 5.2E+00, 1.1E−03; LIPH, 5.4E+00, 1.1E−03; GNB3, 6.3E+00, |
| | 1.2E−02; NPIPA1, 6.6E+00, 8.0E−03; LOC105375434, 8.3E+00, 4.4E−03; MYO1F, 1.2E+01, 3.6E−02; USH2A, 1.5E+01, 3.9E−02; ITGA9, 1.5E+01, 4.6E−03; |
| | MALAT1, 1.7E+01, 6.4E−04; LRP1B, 1.9E+01, 3.7E−02; LOC107986022, 2.1E+01, 3.9E−02; FRY, 2.1E+01, 3.3E−02; FRA10AC1, 2.2E+01, 7.4E−03; CADM1, |
| | 2.5E+01, 3.3E−02; PAM, 2.6E+01, 3.4E−02; PPP2R3A, 2.8E+01, 3.3E−02 |
| 8-1 | RPS8, −1.4E+01, 3.5E−09; RPL5, −1.4E+01, 1.7E−07; RPL10, −1.3E+01, 1.2E−05; CD24, −1.3E+01, 2.2E−10; EEF1A1, −1.3E+01, 1.1E−15; NPM1, −1.3E+01, |
| | 2.2E−10; RPS17, −1.3E+01, 8.5E−06; RPL23, −1.3E+01, 2.3E−05; PTMA, −1.3E+01, 3.9E−16; RPS6, −1.2E+01, 3.5E−09; ACTG1, −1.2E+01, 1.9E−05; RPL35A, |
| | −1.2E+01, 4.5E−05; RPS2, −1.2E+01, 6.8E−05; RPS23, −1.2E+01, 1.9E−05; RPL8, −1.2E+01, 7.9E−05; HNRNPA1, −1.2E+01, 2.4E−06; ACTB, −1.2E+01, 2.5E−09; |
| | FTH1, −1.2E+01, 1.1E−04; YBX1, −1.1E+01, 1.5E−04; RPL7A, −1.1E+01, 7.2E−05; RPL34, −1.1E+01, 2.7E−04; HDAC2, −1.1E+01, 1.3E−04; RPL13, |
| | −1.1E+01, 7.9E−05; RPL28, −1.1E+01, 2.5E−04; CALM1, −1.1E+01, 2.3E−04; RPL10A, −1.1E+01, 1.9E−04; RPL13A, −1.1E+01, 6.2E−06; GJA1, −1.1E+01, 4.1E−04; |
| | HSP90AA1, −1.1E+01, 7.1E−08; RPS5, −1.0E+01, 4.6E−04; RPL24, −1.0E+01, 8.7E−04; LDHB, −1.0E+01, 2.8E−04; RPL12, −1.0E+01, 8.3E−04; RPS9, |
| | −1.0E+01, 1.6E−03; RPL30, −1.0E+01, 1.7E−03; POU5F1, −1.0E+01, 1.6E−03; PABPC1, −1.0E+01, 9.8E−05; HSP90AB1, −1.0E+01, 2.3E−06; NCL, −1.0E+01, |
| | 2.6E−05; RPS14, −1.0E+01, 5.6E−04; TMSB4X, −1.0E+01, 1.3E−03; RPL4, −1.0E+01, 1.9E−04; RPS16, −9.9E+00, 9.1E−04; RPLPO, −9.8E+00, 1.3E−03; SET, |
| | −9.7E+00, 9.1E−04; ENO1, −9.7E+00, 2.9E−03; RPLP1, −9.6E+00, 6.4E−05; EIF4G2, −9.6E+00, 1.0E−03; EEF1G, −9.6E+00, 3.8E−03; HSPA8, −9.5E+00, 5.1E−04; |
| | CANX, −9.5E+00, 3.0E−04; TARS1, −9.5E+00, 1.7E−03; TPT1, −9.5E+00, 3.5E−03; LIN28A, −9.4E+00, 1.2E−04; RPL3, −9.4E+00, 8.1E−04; RPS12, |
| | −9.3E+00, 6.0E−03; FTL, −9.3E+00, 7.0E−03; RPLP2, −9.3E+00, 7.2E−03; RPL18A, −9.2E+00, 4.4E−03; RPL15, −9.1E+00, 4.9E−03; HSPD1, −9.1E+00, 6.3E−04; |
| | RPL21, −9.0E+00, 8.0E−03; RPS28, −9.0E+00, 3.3E−03; MAP1B, −9.0E+00, 1.2E−02; PFN1, −9.0E+00, 7.7E−03; RPL31, −9.0E+00, 5.4E−03; RPS3, −9.0E+00, |
| | 7.7E−03; RPL26, −9.0E+00, 9.2E−03; RPL9, −8.9E+00, 9.7E−03; RPL14, −8.9E+00, 1.1E−02; NACA, −8.9E+00, 1.1E−02; HMGA1, −8.9E+00, 7.4E−03; RPL32, |
| | −8.8E+00, 1.6E−02; NASP, −8.8E+00, 3.5E−03; RPSA, −8.7E+00, 1.1E−02; RPS18, −8.6E+00, 3.8E−03; SRSF3, −8.6E+00, 1.1E−02; RPS15A, −8.5E+00, |
| | 1.5E−02; GAPDH, −8.5E+00, 1.3E−03; EEF2, −8.5E+00, 1.1E−02; RPL29, −8.5E+00, 1.2E−02; H2AZ1, −8.5E+00, 1.0E−02; NORAD, −8.4E+00, 1.5E−02; EID1, |
| | −8.3E+00, 3.5E−03; RPS13, −8.3E+00, 1.7E−02; GSTP1, −8.3E+00, 3.2E−02; RPL11, −8.2E+00, 7.7E−03; RPS7, −8.2E+00, 2.5E−02; RPS11, −8.2E+00, 3.4E−02; |
| | TPM3, −8.2E+00, 2.5E−02; HNRNPE, −8.2E+00, 9.0E−03; ATP8, −8.2E+00, 1.6E−02; PRDX1, −8.2E+00, 2.6E−02; RPS27A, −8.1E+00, 2.4E−02; CSDE1, |
| | −8.1E+00, 2.0E−02; TPI1, −8.1E+00, 2.9E−02; ATP5F1B, −8.1E+00, 2.5E−02; ZNF483, −8.1E+00, 9.2E−03; SLC38A1, −8.0E+00, 3.4E−02; H3F3B, −8.0E+00, |
| | 2.6E−02; PDIA6, −8.0E+00, 2.2E−02; TDGF1, −7.9E+00, 3.0E−02; RPS24, −7.9E+00, 3.0E−02; L1TD1, −7.9E+00, 1.8E−03; MYL6, −7.8E+00, 4.1E−02; YWHAG, |
| | −7.6E+00, 4.4E−02; NDUFS5, −7.6E+00, 1.5E−02; DNAJA1, −7.6E+00, 2.9E−02; NARS1, −7.5E+00, 2.0E−02; QSER1, −7.2E+00, 3.4E−02; ATP5PD, −7.2E+00, |
| | 2.2E−02; PSMB1, −6.5E+00, 4.2E−02; SF3A3, −6.4E+00, 4.3E−02; COX1, −6.3E+00, 2.3E−07; IFITM1, −6.2E+00, 2.8E−02; COX7B, −5.8E+00, 9.6E−03; COX3, |
| | −5.7E+00, 2.7E−04; TRUB1, −5.5E+00, 1.4E−02; ND5, −4.9E+00, 2.9E−02; ATP6, −4.6E+00, 1.2E−02; ND4, −4.6E+00, 8.1E−04; CYTB, −4.5E+00, 2.4E−02; |
| | ITIH5, −2.3E+00, 1.2E−02; LOC105370761, 6.4E−01, 1.6E−02; NINJ2, 7.5E−01, 2.8E−02; MIR4432HG, 9.3E−01, 2.2E−02; LOC105379412, 1.5E+00, 2.3E−02; |
| | LOC105377359, 1.7E+00, 3.8E−02; LINC00951, 1.9E+00, 2.5E−02; PAPPA2, 2.0E+00, 2.0E−02; LOC105377561, 2.1E+00, 2.7E−02; LOC107983968, |
| | 2.1E+00, 2.9E−02; VWA2, 2.2E+00, 2.5E−02; SPINK14, 2.2E+00, 4.6E−02; SRP14-AS1, 2.3E+00, 3.6E−02; SYBU, 2.4E+00, 2.5E−03; NALCN-AS1, 2.6E+00, |
| | 1.8E−02; LOC105373187, 2.7E+00, 3.3E−02; LOC105373455, 2.8E+00, 1.7E−02; LRRTM3, 3.1E+00, 4.6E−02; LINC00989, 3.2E+00, 4.4E−02; RELL1, |
| | 3.2E+00, 2.1E−02; FSIP2, 3.5E+00, 1.7E−02; ATP10A, 3.5E+00, 2.0E−02; LOC339622, 3.6E+00, 1.1E−02; SETD4, 4.1E+00, 1.9E−02; LOC105370737, |
| | 4.1E+00, 1.3E−04; DCBLD1, 4.2E+00, 5.5E−05; LOC101927896, 4.4E+00, 5.4E−03; LOC107984393, 4.6E+00, 2.1E−03; IFRD1, 4.8E+00, 4.7E−02; NPHP4, |
| | 4.8E+00, 3.5E−02; PRKCE, 4.8E+00, 5.7E−03; LINC00632, 4.9E+00, 2.8E−02; KIF26B, 4.9E+00, 3.3E−02; LOC105373703, 5.0E+00, 3.5E−02; AASS, |
| | 5.2E+00, 8.3E−03; GRM7, 5.8E+00, 1.6E−02; LOC107986324, 5.8E+00, 1.0E−02; LOC105378308, 6.0E+00, 9.1E−03; LOC105377700, 6.1E+00, 6.9E−03; |
| | FOXN3, 6.2E+00, 7.6E−04; SLC30A7, 6.2E+00, 4.2E−02; WASF1, 6.3E+00, 2.0E−02; ECHDC2, 6.3E+00, 1.8E−03; FAM155A, 6.3E+00, 4.5E−02; PCSK6, |
| | 6.3E+00, 1.3E−02; NR3C2, 6.3E+00, 2.9E−02; PPP1R16B, 6.4E+00, 8.0E−03; B3GALT1, 6.4E+00, 8.8E−03; VCAN, 6.5E+00, 7.8E−03; RHOT1, 6.5E+00, |
| | 1.2E−02; WDR37, 6.6E+00, 1.2E−03; NHS, 6.6E+00, 4.9E−02; SNTG2, 6.7E+00, 3.1E−02; ST7, 6.8E+00, 2.2E−02; SCFD2, 6.8E+00, 5.0E−02; RALGAPB, 6.8E+00, |
| | 1.0E−02; VAV3, 7.0E+00, 4.2E−02; NFIA, 7.0E+00, 3.7E−03; FSTL4, 7.1E+00, 1.3E−02; GRIN2A, 7.1E+00, 3.6E−03; MYO5B, 7.2E+00, 1.6E−02; MTTP, |
| | 7.2E+00, 5.6E−06; CDH6, 7.2E+00, 3.5E−02; PCSK5, 7.3E+00, 4.1E−02; RCOR1, 7.3E+00, 5.0E−02; NOS1AP, 7.3E+00, 1.6E−02; FLRT2, 7.3E+00, 2.1E−03; |
| | RERE, 7.3E+00, 2.6E−02; RABGAP1L, 7.4E+00, 4.9E−03; FAM13B, 7.4E+00, 1.7E−02; FGD4, 7.4E+00, 3.1E−02; CECR2, 7.4E+00, 6.3E−03; ACSS3, 7.5E+00, |
| | 1.5E−02; SYN3, 7.6E+00, 9.6E−03; VWA8, 7.6E+00, 2.6E−02; APP, 7.8E+00, 3.8E−02; JPX, 7.8E+00, 4.5E−02; CDK8, 7.8E+00, 1.6E−02; PTK2, 7.9E+00, |
| | 1.0E−02; PHKB, 7.9E+00, 1.0E−02; PIAS1, 7.9E+00, 1.5E−02; MACROD2, 7.9E+00, 3.3E−02; GXYLT2, 7.9E+00, 1.5E−04; PTPN4, 7.9E+00, 3.3E−02; TMTC2, |
| | 7.9E+00, 3.4E−02; TTC17, 7.9E+00, 3.4E−02; DLGAP1, 7.9E+00, 7.1E−04; DLG2, 8.0E+00, 3.8E−02; MALAT1, 8.0E+00, 3.4E−10; ADGRA3, 8.0E+00, 2.4E−02; |
| | MBTD1, 8.1E+00, 1.2E−02; FTX, 8.2E+00, 1.6E−03; PHACTR2, 8.2E+00, 1.9E−02; TRIM24, 8.2E+00, 9.6E−03; ZNF577, 8.2E+00, 1.5E−03; HERC1, |
| | 8.2E+00, 3.6E−02; MSH3, 8.3E+00, 3.3E−03; TASP1, 8.4E+00, 1.8E−02; SIPA1L3, 8.4E+00, 1.7E−02; KLF8, 8.4E+00, 1.6E−02; TENM3, 8.4E+00, 9.1E−03; |
| | ADGRL2, 8.5E+00, 4.7E−03; MAP2K5, 8.5E+00, 2.0E−03; RANBP17, 8.6E+00, 2.6E−02; DAB1, 8.6E+00, 2.1E−04; PSPC1, 8.6E+00, 1.2E−02; RUNX1T1, |
| | 8.6E+00, 2.0E−02; GAB1, 8.7E+00, 5.7E−03; LOC101929194, 8.7E+00, 1.7E−02; CAMK1D, 8.7E+00, 1.0E−02; LOC339862, 8.7E+00, 1.5E−02; EDIL3, |
| | 8.7E+00, 2.0E−02; GRID2, 8.8E+00, 5.8E−16; LOC105370482, 8.8E+00, 8.5E−03; SPATS2L, 8.8E+00, 1.0E−02; PPP1R9A, 8.8E+00, 7.7E−03; KLF12, |
| | 8.9E+00, 1.2E−02; DENND1A, 8.9E+00, 1.0E−02; SVIL, 8.9E+00, 1.1E−03; MCF2L, 8.9E+00, 6.5E−04; SMYD3, 8.9E+00, 9.2E−03; MBD5, 8.9E+00, 1.1E−02; |
| | NEAT1, 9.0E+00, 8.1E−04; LOC101929231, 9.0E+00, 2.8E−05; DOCK3, 9.0E+00, 1.4E−02; PTPRG, 9.0E+00, 1.1E−06; TRAPPC9, 9.0E+00, 3.4E−03; |
| | PHACTR1, 9.1E+00, 1.3E−02; MED13L, 9.1E+00, 9.6E−03; ABCA1, 9.1E+00, 7.5E−03; AUH, 9.2E+00, 2.5E−03; PTPRD, 9.2E+00, 2.9E−03; C5orf46, |
| | 9.2E+00, 5.9E−04; ZYG11A, 9.4E+00, 7.5E−05; TBC1D22A, 9.4E+00, 4.6E−03; PLCH1, 9.4E+00, 3.4E−03; ERBIN, 9.6E+00, 3.6E−03; MAML2, 9.6E+00, 4.7E−03; |
| | TMEM135, 9.6E+00, 1.8E−03; PLCB1, 9.6E+00, 3.7E−03; DMXL1, 9.7E+00, 2.5E−03; PRKCA, 9.7E+00, 4.6E−03; PRICKLE2, 9.8E+00, 1.5E−03; LPP, |
| | 9.9E+00, 9.1E−04; CFTR, 1.0E+01, 9.5E−04; ZNF827, 1.0E+01, 1.6E−03; ACOXL, 1.0E+01, 4.7E−03; ADGRV1, 1.0E+01, 3.2E−05; ATXN7L1, 1.0E+01, 9.7E−04; |
| | RASSF8, 1.1E+01, 3.4E−05; AUTS2, 1.1E+01, 1.4E−09; KCNQ10T1, 1.1E+01, 1.2E−04; PLEKHA5, 1.1E+01, 1.6E−07; CBLB, 1.1E+01, 4.4E−04; FBXW11, |
| | 1.1E+01, 1.2E−04; SOX2-OT, 1.1E+01, 6.7E−04; KIAA0825, 1.1E+01, 4.4E−04; MGAT5, 1.1E+01, 3.7E−04; NRCAM, 1.2E+01, 2.9E−06; GMDS, 1.2E+01, |
| | 9.1E−05; PREX2, 1.2E+01, 2.1E−04; KANK1, 1.2E+01, 1.1E−04; BMP2K, 1.2E+01, 1.6E−05; TRPS1, 1.2E+01, 9.4E−05; COL8A1, 1.2E+01, 3.5E−09; PAN3, |
| | 1.3E+01, 1.2E−05; ADCY2, 1.3E+01, 1.6E−04; ADGRL3, 1.3E+01, 2.2E−11; ELF1, 1.3E+01, 3.6E−07; AGBL4, 1.3E+01, 1.1E−07; ZC3H12B, 1.3E+01, |
| | 1.6E−05; CNKSR3, 1.3E+01, 1.4E−05; TMEM132D, 1.3E+01, 2.0E−07; FOXP1, 1.3E+01, 7.1E−07; KDM4C, 1.4E+01, 1.4E−06; KAZN, 1.5E+01, 2.1E−07; CUBN, |
| | 1.5E+01, 9.9E−09; OPCML, 1.6E+01, 6.6E−11; VIPR2, 1.7E+01, 5.2E−12; CTNNA3, 1.7E+01, 7.6E−09; LOC107987166, 1.7E+01, 4.4E−08; MPPED2, |
| | 1.8E+01, 5.2E−10; AFF2, 1.8E+01, 2.5E−10; RASGRF2, 1.8E+01, 2.1E−11; LOC105370777, 2.0E+01, 6.6E−12 |
| 9-0 | LOC107986777, −2.5E+01, 2.3E−04; LOC101927668, −2.2E+01, 3.6E−03; PCAT14, −2.0E+01, 7.3E−03; APELA, −1.8E+01, 4.3E−02; GRID2, −1.1E+01, 2.9E−04; |
| | GHRL, 1.6E+00, 5.4E−05; LINC00867, 1.6E+00, 5.4E−05; LOC102723750, 1.6E+00, 5.4E−05; LOC105370425, 1.6E+00, 5.4E−05; LOC105371228, |
| | 1.6E+00, 5.4E−05; LOC105376199, 1.6E+00, 5.4E−05; LOC105378470, 1.6E+00, 5.4E−05; PHF11, 1.6E+00, 5.4E−05; LOC107986870, 1.7E+00, 2.5E−02; |
| | HOPX, 1.7E+00, 4.2E−03; TSLP, 1.8E+00, 8.0E−04; DCAF13P3, 2.0E+00, 7.6E−05; LOC105376866, 2.1E+00, 2.5E−02; LOC105372133, 2.7E+00, 3.9E−04; |
| | SLC10A3, 2.9E+00, 2.0E−02; ATP12A, 3.6E+00, 3.0E−02; VGLL1, 4.4E+00, 5.3E−03; SLC50A1, 6.5E+00, 2.5E−02; RALGDS, 6.9E+00, 2.1E−02; ANXA3, |
| | 9.1E+00, 9.2E−03; PGAP2, 9.9E+00, 4.2E−02; SLC39A8, 1.0E+01, 2.3E−02; PGGT1B, 1.0E+01, 4.1E−02; SLC8A1, 1.1E+01, 1.9E−02; LRP1B, 1.2E+01, 2.5E−02; |
| | ABCG2, 1.3E+01, 5.4E−05; VSNL1, 1.5E+01, 4.4E−02; FREM2, 1.6E+01, 3.0E−02; SAE1, 1.8E+01, 1.9E−02; TLE4, 1.8E+01, 1.9E−02; SLC2A3, 1.8E+01, |
| | 1.5E−02; AHNAK, 2.0E+01, 1.9E−02; ITGB1, 2.0E+01, 2.8E−03; GREB1L, 2.2E+01, 2.3E−04; MAGI3, 2.3E+01, 1.6E−02; DSP, 2.6E+01, 2.3E−04; PRTG, |
| | 2.9E+01, 1.6E−08; FBN2, 3.0E+01, 6.1E−05; HAPLN1, 3.8E+01, 2.1E−10 |
| 9-1 | DMD, −1.1E+01, 2.6E−04; MAGI3, −1.0E+01, 9.1E−05; FN1, −9.1E+00, 1.8E−02; PRKG1, −9.0E+00, 5.0E−04; SSBP2, −7.9E+00, 1.4E−03; PCDH11X, −6.9E+00, |
| | 7.1E−03; NLGN4X, −6.6E+00, 2.0E−02; DOK6, −5.4E+00, 3.7E−02; HS6ST2, −5.1E+00, 2.6E−02; LRRN1, −5.1E+00, 3.8E−02; PROM2, 6.6E−01, 4.3E−02; |
| | LINC00989, 6.9E−01, 3.8E−02; HSPE1-MOB4, 7.0E−01, 4.1E−02; IL36RN, 8.1E−01, 4.2E−02; LOC107985980, 8.8E−01, 3.9E−02; GORASP1, 9.3E−01, 4.3E−02; |
| | LOC105373369, 9.7E−01, 3.8E−02; LOC107984256, 1.0E+00, 2.2E−02; LINC01314, 1.0E+00, 3.5E−02; MYOCD, 1.0E+00, 4.9E−02; SCIN, 1.1E+00, |
| | 3.5E−03; MMP19, 1.1E+00, 4.6E−02; LOC101927354, 1.1E+00, 3.7E−02; DHRS9, 1.1E+00, 4.9E−02; TSEN34, 1.1E+00, 4.3E−02; GBP2, 1.1E+00, 1.8E−03; |
| | ENG, 1.3E+00, 3.5E−02; GPR78, 1.3E+00, 3.5E−02; LOC107984916, 1.4E+00, 4.6E−02; CBR3-AS1, 1.5E+00, 1.8E−03; CCR7, 1.6E+00, 2.5E−02; DRC3, |
| | 1.6E+00, 2.8E−03; LOC107986087, 1.6E+00, 5.3E−04; LOC105373347, 1.6E+00, 4.4E−03; CD96, 1.6E+00, 9.8E−04; MFAP5, 1.7E+00, 2.2E−02; SHC3, |
| | 1.8E+00, 2.2E−02; LOC107987295, 1.8E+00, 4.7E−03; PTPN5, 1.8E+00, 3.5E−02; FAP, 1.8E+00, 2.1E−04; PGF, 1.8E+00, 8.8E−03; SH3TC2, 1.8E+00, 3.8E−03; |
| | NBPF3, 1.8E+00, 4.1E−02; ANGPT4, 1.9E+00, 1.7E−02; LOC105377872, 1.9E+00, 2.9E−03; ELF5, 1.9E+00, 4.1E−02; CLIC3, 2.0E+00, 4.5E−04; KIFC3, |
| | 2.0E+00, 2.7E−02; FYB1, 2.0E+00, 1.3E−03; GCM1, 2.0E+00, 1.5E−03; TINAGL1, 2.1E+00, 1.2E−05; ERI2, 2.1E+00, 2.2E−02; PTPRE, 2.3E+00, 1.6E−02; |
| | GABRE, 2.3E+00, 3.1E−03; LOC105376942, 2.3E+00, 9.6E−03; SNAP91, 2.3E+00, 1.9E−03; CSF3R, 2.4E+00, 1.9E−04; DPF3, 2.4E+00, 2.8E−03; DGKA, |
| | 2.5E+00, 2.6E−04; LOC105376958, 2.5E+00, 4.4E−03; CGA, 2.6E+00, 2.6E−02; SPRR3, 2.7E+00, 1.8E−03; B3GNT5, 2.7E+00, 1.6E−03; SMPDL3A, 2.7E+00, |
| | 2.3E−03; GLDN, 2.7E+00, 4.1E−03; NR2F2-AS1, 2.8E+00, 3.7E−02; TENT5A, 2.8E+00, 4.6E−05; ACAD9, 2.8E+00, 2.7E−02; PMEL, 2.8E+00, 9.1E−03; |
| | HS3ST3B1, 2.9E+00, 4.3E−04; LOC646762, 2.9E+00, 6.5E−03; CREG1, 2.9E+00, 6.8E−03; ARAP2, 2.9E+00, 2.7E−02; SKAP2, 3.0E+00, 2.6E−02; C5orf17, |
| | 3.0E+00, 7.3E−06; ZBTB38, 3.0E+00, 7.5E−03; GUCY1A2, 3.0E+00, 4.3E−02; ANKMY2, 3.1E+00, 2.7E−02; SLC2A14, 3.1E+00, 5.2E−05; ACER2, 3.1E+00, |
| | 2.6E−02; TMEM200A, 3.1E+00, 1.7E−02; CDK2, 3.1E+00, 7.8E−03; GRHL3, 3.2E+00, 3.8E−02; CYP7B1, 3.2E+00, 3.6E−02; SLC16A6, 3.2E+00, 1.3E−04; |
| | LOC105377067, 3.3E+00, 9.2E−03; LINC01340, 3.3E+00, 4.1E−02; CHST3, 3.3E+00, 3.8E−03; MCTP2, 3.4E+00, 8.3E−03; LOC101927768, 3.4E+00, |
| | 1.6E−02; LOC105374689, 3.4E+00, 5.0E−04; CPEB4, 3.5E+00, 4.9E−02; LOC105374037, 3.5E+00, 8.0E−03; MALAT1, 3.5E+00, 2.2E−02; MGLL, 3.5E+00, 6.2E−06; |
| | MACIR, 3.5E+00, 2.7E−02; C2orf72, 3.5E+00, 1.5E−05; HOPX, 3.6E+00, 2.0E−07; GABBR2, 3.7E+00, 7.4E−07; LOC105377509, 3.7E+00, 3.5E−03; |
| | P2RY6, 3.7E+00, 4.2E−04; XCR1, 3.8E+00, 2.4E−04; PTN, 3.9E+00, 4.0E−02; ATP13A4, 3.9E+00, 1.9E−07; CDKN1C, 4.0E+00, 9.3E−04; ATP10B, 4.0E+00, |
| | 4.6E−05; SLC25A37, 4.0E+00, 1.6E−02; RIN3, 4.1E+00, 2.9E−03; RHOU, 4.1E+00, 5.3E−03; FLVCR2, 4.2E+00, 5.0E−04; PPARD, 4.4E+00, 2.6E−04; FHL2, |
| | 4.5E+00, 3.3E−05; NR2F2, 4.5E+00, 2.8E−03; KATNBL1, 4.6E+00, 4.1E−02; ARHGEF7, 4.6E+00, 4.3E−02; CLCN3, 4.6E+00, 3.7E−02; PLCXD3, 4.6E+00, |
| | 8.2E−10; IL1RAPL2, 4.7E+00, 6.6E−04; GRHL1, 4.7E+00, 1.8E−02; TACC1, 4.7E+00, 1.9E−02; CDC14B, 4.7E+00, 3.7E−02; TSPAN5, 4.8E+00, 3.0E−02; |
| | MB21D2, 4.8E+00, 4.1E−02; LOC101929528, 4.9E+00, 5.9E−10; LCP1, 4.9E+00, 3.1E−05; LAMA1, 4.9E+00, 3.1E−02; KRT23, 4.9E+00, 2.6E−07; SNX29, |
| | 4.9E+00, 6.1E−03; TRHDE, 4.9E+00, 5.8E−05; EVI5, 5.0E+00, 3.5E−02; ATP11B, 5.1E+00, 7.8E−03; MPRIP, 5.1E+00, 4.0E−02; ARL8B, 5.1E+00, 1.8E−03; |
| | RALBP1, 5.1E+00, 2.2E−02; NR3C2, 5.2E+00, 1.7E−02; B4GALT1, 5.2E+00, 2.6E−03; USP24, 5.3E+00, 4.4E−02; CRTC3, 5.3E+00, 1.9E−07; KLHL29, |
| | 5.3E+00, 2.7E−04; EGFR, 5.3E+00, 3.0E−02; ADCY10, 5.3E+00, 1.2E−05; ZBTB7C, 5.3E+00, 8.9E−08; SH3KBP1, 5.3E+00, 8.0E−03; LOC105370504, |
| | 5.4E+00, 4.1E−02; MYO9A, 5.4E+00, 3.0E−02; TNKS2, 5.6E+00, 3.5E−02; GLDC, 5.6E+00, 1.3E−02; RASA1, 5.6E+00, 5.4E−03; KHDRBS3, 5.6E+00, 2.5E−03; |
| | GAB1, 5.6E+00, 2.1E−02; AAK1, 5.6E+00, 3.5E−02; ST8SIA4, 5.6E+00, 1.8E−11; PSAP, 5.7E+00, 3.8E−02; MME, 5.7E+00, 1.3E−03; ARHGAP24, |
| | 5.7E+00, 8.0E−03; ACOX1, 5.8E+00, 4.6E−05; FHDC1, 5.8E+00, 5.2E−05; NEK7, 5.9E+00, 2.8E−03; WSB1, 5.9E+00, 3.0E−02; EPB41L4B, 5.9E+00, 5.9E−05; |
| | SHANK2, 6.0E+00, 4.3E−02; SLC39A8, 6.0E+00, 4.1E−05; BZW2, 6.0E+00, 1.2E−02; HOOK3, 6.1E+00, 4.4E−03; NABP1, 6.1E+00, 5.0E−06; FSTL1, |
| | 6.1E+00, 3.5E−02; REV3L, 6.1E+00, 1.2E−02; CD47, 6.1E+00, 3.0E−06; ST3GAL1, 6.2E+00, 7.3E−06; ANXA1, 6.2E+00, 1.2E−02; TRAPPC9, 6.2E+00, 6.5E−03; |
| | TNFRSF21, 6.2E+00, 2.2E−03; EPS8, 6.2E+00, 3.0E−04; PAPOLA, 6.3E+00, 3.6E−02; ERRFI1, 6.3E+00, 1.7E−03; MTNR1B, 6.3E+00, 2.3E−12; TMCC1, |
| | 6.3E+00, 1.4E−02; VAV3, 6.4E+00, 4.9E−05; ANK3, 6.5E+00, 8.1E−03; LOC101928635, 6.6E+00, 3.6E−08; CSGALNACT1, 6.6E+00, 3.3E−07; GATA3, |
| | 6.7E+00, 3.8E−07; TBC1D9, 6.7E+00, 1.4E−05; XYLT1, 6.7E+00, 2.5E−03; ENC1, 6.8E+00, 1.0E−07; NEBL, 6.8E+00, 8.0E−03; NAV2, 6.8E+00, 8.7E−03; |
| | RASAL2, 6.9E+00, 3.4E−03; CRYBG1, 6.9E+00, 1.9E−07; MYOF, 7.0E+00, 3.1E−05; WDR70, 7.1E+00, 2.6E−04; NRP1, 7.2E+00, 3.0E−08; SASH1, 7.4E+00, |
| | 2.3E−05; ARRB1, 7.4E+00, 1.2E−04; MBNL3, 7.4E+00, 3.0E−06; ELMO1, 7.4E+00, 1.7E−03; CLMP, 7.7E+00, 1.4E−05; MAN1A2, 7.8E+00, 6.1E−04; MBNL2, |
| | 7.8E+00, 2.8E−05; LOC105378334, 7.8E+00, 1.4E−07; S100A9, 7.9E+00, 2.1E−08; MAML2, 7.9E+00, 1.9E−04; CYP19A1, 8.3E+00, 1.3E−08; PIK3C2G, |
| | 8.4E+00, 5.8E−10; MUC15, 8.6E+00, 2.2E−09; ADGRL3, 8.6E+00, 2.6E−04; TANC2, 8.6E+00, 7.0E−05; LOC107986945, 8.7E+00, 1.3E−13; TMEM117, |
| | 8.8E+00, 1.8E−05; SLC7A2, 8.9E+00, 8.3E−05; RBM47, 9.0E+00, 5.6E−05; PEG10, 9.0E+00, 6.8E−04; SYNE2, 9.1E+00, 2.0E−05; THRB, 9.3E+00, 1.1E−05; |
| | LOC105374039, 9.4E+00, 1.8E−13; MAML3, 9.5E+00, 1.5E−04; STS, 9.5E+00, 7.0E−12; PPARG, 1.0E+01, 2.1E−13; ACSM1, 1.0E+01, 9.1E−18; EPAS1, |
| | 1.1E+01, 4.2E−11; PDE10A, 1.1E+01, 1.6E−09; WWC1, 1.1E+01, 8.2E−10; NEAT1, 1.1E+01, 2.0E−05; LINC00278, 1.2E+01, 7.8E−14; PRKCH, 1.4E+01, |
| | 4.6E−14; NHS, 1.4E+01, 1.1E−12; CCSER1, 1.9E+01, 2.0E−17 |
| 9-2 | UNC5C, 1.7E+01, 2.6E−02; COL5A2, 1.7E+01, 1.2E−02; CYP11A1, 1.6E+01, 2.9E−05; WNT8A, 1.1E+01, 8.9E−03; SPATA16, 8.1E+00, 1.2E−02 |
| 10 | FN1, −2.9E+00, 7.1E−08; CDH2, −2.5E+00, 2.4E−06; H2AZ1, −2.5E+00, 9.7E−06; HSP90B1, −2.5E+00, 1.8E−07; CD63, −2.2E+00, 9.1E−05; TOP2A, −2.2E+00, |
| | 2.7E−04; H3C2, −2.2E+00, 2.4E−03; HMGB2, −2.1E+00, 3.5E−04; CSRP2, −2.1E+00, 2.4E−06; H1-5, −2.0E+00, 3.8E−03; XRCC6, −2.0E+00, 1.1E−03; PEG10, |
| | −2.0E+00, 1.5E−04; ACTB, −2.0E+00, 5.8E−06; SMC4, −2.0E+00, 7.8E−04; FTL, −2.0E+00, 1.9E−03; SPARC, −1.9E+00, 3.2E−04; HSPA5, −1.9E+00, 5.2E−03; |
| | H4C3, −1.8E+00, 1.8E−02; YWHAQ, −1.8E+00, 7.5E−03; ATP5F1A, −1.8E+00, 5.3E−03; CENPF, −1.8E+00, 6.4E−03; CLIC1, −1.7E+00, 4.0E−04; STMN1, |
| | −1.7E+00, 2.4E−02; MYH10, −1.7E+00, 7.6E−03; EPB41L2, −1.7E+00, 8.7E−03; VCAN, −1.6E+00, 1.5E−02; DIAPH3, −1.6E+00, 1.7E−02; TMSB4X, −1.6E+00, |
| | 4.2E−02; GJA1, −1.6E+00, 2.4E−02; PODXL, −1.6E+00, 1.6E−02; SEPTIN11, −1.6E+00, 9.0E−03; DEK, −1.6E+00, 2.9E−02; EIF3J, −1.6E+00, 4.1E−03; SQLE, |
| | −1.6E+00, 1.3E−02; WDR43, −1.6E+00, 1.3E−02; PCNP, −1.5E+00, 1.3E−02; RPL12, −1.5E+00, 4.2E−02; NCL, −1.5E+00, 4.1E−03; COL4A1, −1.5E+00, 8.4E−03; |
| | HNRNPU, −1.5E+00, 1.8E−02; DPYSL2, −1.5E+00, 2.0E−02; CCNB1, −1.5E+00, 3.5E−02; LDHB, −1.5E+00, 4.7E−02; TUBA1A, −1.4E+00, 2.8E−03; NTS, |
| | −1.4E+00, 2.9E−06; RPS18, −1.4E+00, 4.3E−02; FADS1, −1.4E+00, 3.3E−02; NFE2L3, −1.4E+00, 4.5E−04; NNAT, −1.4E+00, 5.5E−03; HSPD1, −1.4E+00, 5.0E−02; |
| | PTMA, −1.4E+00, 3.8E−04; CYB5B, −1.3E+00, 2.8E−02; TPM2, −1.3E+00, 2.7E−02; CENPK, −1.3E+00, 1.6E−02; CKAP2, −1.3E+00, 4.3E−02; CTNNBL1, |
| | −1.2E+00, 4.3E−03; UBA2, −1.2E+00, 4.6E−02; CDCA2, −1.2E+00, 1.1E−02; H3C10, −1.2E+00, 3.7E−02; PPIB, −1.2E+00, 2.5E−02; HAUS1, −1.2E+00, 1.9E−02; |
| | RRM2, −1.2E+00, 3.8E−02; ANKRD1, −1.2E+00, 2.3E−03; PTTG1, −1.2E+00, 4.0E−02; BRCA1, −1.2E+00, 3.6E−02; VIM, −1.1E+00, 2.4E−02; HIRA, −1.1E+00, |
| | 1.9E−02; POLQ, −1.1E+00, 2.8E−02; DIPK1A, −1.1E+00, 7.0E−04; NIM1K, −1.1E+00, 4.8E−06; LOC105378008, −1.1E+00, 1.9E−08; H3C7, −1.1E+00, 2.3E−02; |
| | ELK3, −1.1E+00, 1.5E−03; SNRPE, −1.1E+00, 4.9E−02; MTCH1, −1.1E+00, 1.0E−02; TGFBI, −1.1E+00, 1.4E−02; PTP4A1, −1.1E+00, 5.0E−02; TNFRSF10B, |
| | −1.1E+00, 3.1E−03; EEF1A1, −1.1E+00, 2.4E−02; LYN, −1.1E+00, 1.1E−02; PMAIP1, −1.0E+00, 4.3E−02; CCDC141, −1.0E+00, 1.5E−03; COL1A1, −9.7E−01, |
| | 3.2E−02; ELP1, −9.5E−01, 1.8E−02; MRS2, −9.4E−01, 3.2E−02; LINC01504, −8.7E−01, 2.4E−06; COL2A1, −8.6E−01, 2.0E−02; RHOBTB3, −8.6E−01, 4.7E−02; |
| | BICC1, −8.6E−01, 8.8E−04; LOC102723704, −8.5E−01, 1.9E−04; COL5A2, −8.4E−01, 2.3E−02; EML6, −8.2E−01, 1.2E−03; MRPS17, −8.2E−01, 2.0E−02; ABCF2, |
| | −8.2E−01, 1.9E−02; GALM, −8.0E−01, 4.6E−02; KCNQ5, −7.9E−01, 1.9E−03; ZBTB2, −7.8E−01, 1.8E−02; ZEB2, −7.7E−01, 3.4E−02; POLG2, −7.7E−01, 1.4E−02; |
| | FBN1, −7.1E−01, 1.6E−02; ZEB1, −7.1E−01, 2.6E−02; FRMD3, −7.0E−01, 1.3E−02; DNAH12, −7.0E−01, 3.8E−05; CPM, −6.9E−01, 1.1E−03; PCDH17, −6.8E−01, |
| | 2.5E−03; CMYA5, −6.8E−01, 4.0E−03; SCD5, −6.7E−01, 1.2E−02; SNX31, −6.6E−01, 3.7E−05; CPQ, −6.2E−01, 1.4E−02; LGR5, −6.2E−01, 1.9E−03; TFPI, |
| | −6.1E−01, 4.0E−03; MSH4, −5.4E−01, 4.9E−03; FREM3, −5.2E−01, 2.4E−04; SYTL3, −5.1E−01, 2.2E−02; LOC105370575, −5.1E−01, 6.0E−03; ADCY10, −5.0E−01, 4.5E−03; |
| | LOC105369873, −5.0E−01, 2.5E−04; MAP3K19, −4.9E−01, 2.2E−03; NKD1, −4.7E−01, 3.8E−02; COLEC12, −4.7E−01, 4.7E−02; OVOS2, −4.5E−01, 1.0E−03; |
| | ADAM32, −4.5E−01, 1.4E−02; KLB, −4.4E−01, 8.8E−03; C8orf89, −4.2E−01, 2.3E−02; LOC105378316, −4.1E−01, 7.8E−04; LOC101928012, −4.1E−01, 6.0E−03; |
| | KIAA0319, −3.9E−01, 2.3E−02; LOC105377901, −3.9E−01, 3.7E−02; LOC101927156, −3.9E−01, 3.1E−02; ARHGEF6, −3.7E−01, 2.6E−02; RPS10−NUDT3, |
| | −3.7E−01, 4.1E−02; LINC01507, −3.5E−01, 1.2E−02; POF1B, −3.4E−01, 2.7E−02; ITGAM, −3.4E−01, 2.5E−02; LOC101929505, −3.3E−01, 1.3E−02; DAPP1, |
| | −3.3E−01, 2.7E−02; MILR1, −3.2E−01, 1.0E−02; APLNR, −3.1E−01, 3.6E−02; IMPG1, −2.7E−01, 3.3E−02; TMEM236, −2.7E−01, 4.2E−02; MRC1, −2.7E−01, 4.4E−02; |
| | CYP2C18, −2.6E−01, 2.2E−02; LINC01344, −2.5E−01, 4.0E−02; HSPE1-MOB4, −2.4E−01, 3.3E−02; LOC105378644, −2.4E−01, 2.7E−02; CARMN, −2.2E−01, |
| | 4.1E−02; LOC105377616, 8.0E−02, 1.9E−02; LOC105374257, 1.1E−01, 2.2E−02; TCTE1, 1.2E−01, 4.7E−02; LOC107987064, 1.4E−01, 4.3E−02; DIO30S, |
| | 1.4E−01, 3.1E−02; LOC107984311, 1.4E−01, 1.3E−02; LOC105369880, 1.5E−01, 4.3E−02; LOC105370844, 1.6E−01, 4.8E−02; RAD51AP2, 1.7E−01, 3.9E−02; |
| | LINC00282, 1.8E−01, 4.2E−02; FGF12-AS3, 1.8E−01, 2.8E−02; LOC100996573, 1.8E−01, 2.8E−02; LOC107984429, 1.9E−01, 8.4E−03; LINC01349, 2.3E−01, |
| | 4.0E−02; LOC101929805, 2.3E−01, 3.2E−02; LINC00333, 2.4E−01, 2.0E−02; LOC105379102, 2.5E−01, 4.6E−02; LOC105377030, 2.6E−01, 2.7E−02; |
| | LOC107986755, 2.7E−01, 2.2E−03; NKPD1, 2.7E−01, 2.5E−04; LOC101927366, 2.8E−01, 4.1E−04; SMN2, 2.9E−01, 2.0E−02; LOC105373813, 2.9E−01, 2.3E−04; |
| | METRNL, 3.0E−01, 4.2E−02; LOC105373716, 3.0E−01, 1.6E−02; ALDH1L1, 3.3E−01, 3.1E−02; LOC102723370, 3.3E−01, 2.0E−02; LOC107986961, 3.4E−01, |
| | 2.6E−02; LOC105375626, 3.4E−01, 4.9E−02; SLC1A2, 3.5E−01, 8.4E−03; LOC105378787, 3.5E−01, 7.2E−03; KIF6, 3.7E−01, 2.4E−02; LOC105378786, |
| | 3.7E−01, 4.6E−03; MEOX1, 3.7E−01, 2.0E−03; GSTM3, 3.8E−01, 3.1E−02; SPRR2F, 3.8E−01, 2.5E−02; LOC105377352, 3.8E−01, 1.2E−02; MAFB, 3.8E−01, |
| | 2.5E−02; ELF5, 3.9E−01, 1.9E−02; LOC105369410, 4.0E−01, 7.0E−03; BRINP3, 4.2E−01, 3.0E−03; LOC642366, 4.2E−01, 3.0E−02; CFAP299, 4.2E−01, |
| | 1.5E−02; PLEKHM1P1, 4.4E−01, 1.4E−02; LOC105372323, 4.4E−01, 5.3E−03; LOC105378789, 4.5E−01, 2.0E−04; LOC107986813, 4.5E−01, 2.1E−02; ITGA9-AS1, |
| | 4.5E−01, 4.2E−02; ONECUT1, 4.6E−01, 8.4E−03; LOC107983981, 4.6E−01, 4.5E−03; LOC107986674, 4.7E−01, 5.3E−03; LOC105374057, 4.7E−01, 3.3E−02; |
| | CCDC191, 4.8E−01, 2.5E−02; LAMA4, 4.9E−01, 6.6E−03; LOC101929077, 5.0E−01, 8.4E−03; LOC107985967, 5.1E−01, 3.3E−02; XDH, 5.1E−01, 1.7E−02; |
| | FSHR, 5.1E−01, 3.9E−03; ZBTB7C, 5.1E−01, 2.7E−02; USH1C, 5.2E−01, 7.5E−03; RTN4RL1, 5.3E−01, 9.5E−04; LOC105377512, 5.6E−01, 6.2E−05; DCLK2, |
| | 5.6E−01, 4.1E−02; ARHGEF26-AS1, 5.6E−01, 1.9E−03; ZMAT1, 5.7E−01, 2.5E−02; NOX4, 5.8E−01, 3.3E−02; LOC101927745, 5.8E−01, 2.1E−04; SPAM1, 5.8E−01, |
| | 3.0E−04; TAC3, 5.8E−01, 2.7E−02; FRK, 5.9E−01, 2.9E−02; LOC105377338, 5.9E−01, 1.0E−03; LOC105370737, 5.9E−01, 1.6E−02; AHSA2P, 6.0E−01, |
| | 4.2E−02; LOC105374505, 6.1E−01, 2.1E−03; DSCAML1, 6.2E−01, 1.6E−03; LOC105370731, 6.4E−01, 2.2E−02; LOC100506885, 6.4E−01, 3.3E−02; PTK2B, |
| | 6.4E−01, 3.7E−02; ERP27, 6.5E−01, 2.1E−02; TXLNB, 6.6E−01, 3.0E−02; XCR1, 6.6E−01, 1.8E−04; LOC105377067, 6.7E−01, 1.2E−05; FTCDNL1, 6.7E−01, |
| | 1.0E−07; MEIS1, 6.8E−01, 1.7E−02; ATP13A4, 6.8E−01, 5.3E−05; ACKR2, 6.9E−01, 3.0E−04; TRIM38, 7.0E−01, 5.7E−03; ZNF552, 7.2E−01, 5.3E−03; |
| | LOC102724687, 7.2E−01, 2.2E−02; SUSD4, 7.3E−01, 7.5E−04; STAC, 7.3E−01, 4.8E−02; CCDC80, 7.3E−01, 4.5E−02; CATSPERG, 7.4E−01, 2.7E−02; MID2, |
| | 7.4E−01, 4.6E−06; PROS1, 7.5E−01, 4.1E−02; RNF180, 7.5E−01, 4.5E−02; LOC100507487, 7.6E−01, 2.6E−03; LOC105747689, 7.7E−01, 7.3E−04; SETD7, |
| | 7.7E−01, 2.6E−03; GPR157, 7.8E−01, 7.4E−08; FAM83B, 7.8E−01, 3.8E−02; PCED1B, 7.9E−01, 1.4E−02; SLCO4C1, 7.9E−01, 3.0E−04; TMPRSS11D, 8.0E−01, |
| | 4.0E−03; LOC105379049, 8.0E−01, 5.3E−03; KIAA0513, 8.0E−01, 2.5E−03; LOC107985779, 8.1E−01, 5.2E−07; LMX1B, 8.2E−01, 1.7E−03; RASGRP1, 8.4E−01, |
| | 7.3E−04; LINC00331, 8.4E−01, 1.7E−05; CYP1B1, 8.5E−01, 1.4E−02; MUC15, 8.6E−01, 9.7E−07; LOC105378531, 8.7E−01, 1.9E−02; ZFHX4, 8.8E−01, |
| | 3.9E−02; LOC105378308, 8.8E−01, 2.6E−03; SORCS2, 8.9E−01, 2.8E−02; EMX2OS, 9.0E−01, 1.9E−06; SYNPR, 9.0E−01, 1.2E−02; ARMCX5−GPRASP2, 9.0E−01, |
| | 8.0E−03; KCTD16, 9.2E−01, 5.3E−05; MYO5C, 9.2E−01, 4.0E−02; SLC35F4, 9.5E−01, 1.0E−03; VWC2L, 9.6E−01, 7.7E−04; LYST, 9.6E−01, 1.8E−05; |
| | DOCK10, 9.7E−01, 3.8E−02; LOC107985783, 9.8E−01, 7.8E−04; ARL4C, 9.8E−01, 7.1E−03; CLEC2D, 9.9E−01, 4.6E−03; RNF144A, 1.0E+00, 3.0E−02; NDST3, |
| | 1.0E+00, 1.8E−06; ZNF493, 1.0E+00, 4.3E−02; SHOC1, 1.0E+00, 2.8E−02; LOC101928278, 1.0E+00, 1.9E−04; MBD2, 1.0E+00, 1.8E−02; LINC00869, |
| | 1.0E+00, 4.1E−04; ARHGAP29, 1.0E+00, 5.6E−03; TCAIM, 1.1E+00, 1.5E−02; KCTD1, 1.1E+00, 2.5E−02; DNAH11, 1.1E+00, 5.0E−02; LOC105378785, |
| | 1.1E+00, 1.3E−09; GRHL3, 1.1E+00, 9.2E−06; SLC2A13, 1.1E+00, 1.4E−02; KLHL13, 1.1E+00, 1.8E−02; GCNT1, 1.1E+00, 4.7E−02; RAP2C-AS1, 1.1E+00, |
| | 7.2E−05; HOXD4, 1.1E+00, 1.4E−03; LOC100132057, 1.1E+00, 7.4E−05; MTM1, 1.1E+00, 9.2E−04; NR3C2, 1.1E+00, 9.0E−03; LPAR1, 1.1E+00, 2.6E−03; |
| | RNF149, 1.1E+00, 8.4E−03; USH2A, 1.1E+00, 9.3E−06; ACVR2A, 1.1E+00, 2.5E−02; TMCC3, 1.1E+00, 4.6E−03; ATP8B1, 1.1E+00, 1.3E−02; ANXA4, |
| | 1.1E+00, 1.1E−03; LDAH, 1.2E+00, 2.3E−02; PLEKHG1, 1.2E+00, 3.8E−03; SPOCK1, 1.2E+00, 7.2E−03; ZNF804A, 1.2E+00, 3.3E−03; HIVEP2, 1.2E+00, |
| | 2.2E−02; PTCHD4, 1.2E+00, 1.9E−02; CPNE3, 1.2E+00, 6.8E−03; LOC105379057, 1.2E+00, 6.4E−04; MECOM, 1.2E+00, 1.9E−03; IGSF3, 1.2E+00, 1.4E−02; |
| | LINC01356, 1.2E+00, 1.9E−04; ADAMTS6, 1.2E+00, 4.3E−02; FOXO3, 1.2E+00, 3.1E−02; ABCG2, 1.2E+00, 3.0E−02; PARK2, 1.2E+00, 6.5E−03; SNRPN, |
| | 1.2E+00, 6.0E−04; ZC3H12C, 1.2E+00, 1.4E−02; GCNT2, 1.3E+00, 1.9E−02; PRLR, 1.3E+00, 7.8E−04; LOC105369165, 1.3E+00, 7.1E−03; SYBU, 1.3E+00, |
| | 8.0E−06; HOOK3, 1.3E+00, 4.3E−02; GRHL1, 1.3E+00, 7.3E−08; LYPLAL1, 1.3E+00, 2.2E−02; SNX13, 1.3E+00, 2.8E−02; CHIC1, 1.3E+00, 6.0E−04; ARMC9, |
| | 1.3E+00, 1.5E−02; TRAPPC9, 1.3E+00, 2.5E−02; VWA8, 1.3E+00, 2.1E−02; DTNB, 1.3E+00, 2.5E−02; ASXL3, 1.3E+00, 1.2E−02; LYPD6B, 1.3E+00, 5.4E−03; |
| | PKP2, 1.3E+00, 1.7E−02; C20orf194, 1.3E+00, 1.6E−03; SUMF1, 1.3E+00, 2.8E−03; HOXD3, 1.3E+00, 4.1E−04; LOC102723638, 1.3E+00, 2.3E−12; |
| | TRANK1, 1.4E+00, 8.1E−04; ABRACL, 1.4E+00, 2.9E−02; FTO, 1.4E+00, 4.0E−02; SH3BGRL2, 1.4E+00, 1.4E−02; CNGB3, 1.4E+00, 3.9E−10; LINC00922, |
| | 1.4E+00, 8.6E−05; IKZF2, 1.4E+00, 4.1E−03; TPO, 1.4E+00, 1.4E−07; LTBP1, 1.4E+00, 2.8E−02; PIBF1, 1.4E+00, 9.9E−03; GPR137C, 1.4E+00, 6.3E−03; |
| | MBNL2, 1.4E+00, 5.5E−04; CBLB, 1.4E+00, 4.0E−02; FAM110B, 1.4E+00, 9.3E−05; LOC101928438, 1.4E+00, 8.1E−05; MCC, 1.4E+00, 2.2E−02; KLF8, |
| | 1.4E+00, 1.6E−02; DOCK1, 1.4E+00, 4.9E−02; FZD6, 1.5E+00, 1.2E−03; ARHGEF38, 1.5E+00, 1.7E−11; COL25A1, 1.5E+00, 3.1E−03; BACH1, 1.5E+00, |
| | 1.3E−02; MAGI3, 1.5E+00, 2.7E−02; MGA, 1.5E+00, 2.8E−02; IL1RAPL1, 1.5E+00, 1.8E−03; UGGT2, 1.5E+00, 2.5E−02; WSB1, 1.5E+00, 8.4E−03; KLF12, 1.5E+00, |
| | 3.3E−02; FLNB, 1.5E+00, 4.6E−02; LOC105377369, 1.5E+00, 1.9E−09; CRACD, 1.5E+00, 1.1E−03; CBR4, 1.5E+00, 3.6E−03; PATJ, 1.5E+00, 4.1E−02; |
| | ANKRD34B, 1.5E+00, 3.0E−08; RASSF3, 1.5E+00, 2.5E−03; TBC1D1, 1.5E+00, 9.7E−03; DPH6-AS1, 1.5E+00, 7.9E−08; BCAS3, 1.5E+00, 1.8E−02; KDM6A, |
| | 1.6E+00, 4.0E−02; GLRA2, 1.6E+00, 1.6E−08; MACROD2, 1.6E+00, 3.8E−03; AFAP1, 1.6E+00, 9.7E−04; ZFYVE16, 1.6E+00, 2.6E−03; SLC39A8, 1.6E+00, |
| | 1.3E−05; SLC16A1, 1.6E+00, 2.5E−02; TMTC1, 1.6E+00, 2.5E−03; EXOC6B, 1.6E+00, 2.4E−02; GLI3, 1.6E+00, 9.8E−03; MAN1A2, 1.7E+00, 2.6E−03; RERE, |
| | 1.7E+00, 9.1E−03; MED12L, 1.7E+00, 8.9E−04; ZNF423, 1.7E+00, 1.2E−02; TMEM132B, 1.7E+00, 1.1E−02; CNTN5, 1.7E+00, 2.6E−07; VPS13C, 1.7E+00, |
| | 1.1E−03; SH3GL2, 1.7E+00, 1.4E−04; NOTCH2, 1.7E+00, 1.0E−04; PCDH11X, 1.7E+00, 1.9E−02; TFAP2B, 1.7E+00, 1.3E−10; RIN2, 1.7E+00, 4.2E−08; |
| | SCHLAP1, 1.7E+00, 2.4E−05; ZNF385D, 1.7E+00, 1.3E−04; RUNX1T1, 1.7E+00, 7.8E−04; EGFEM1P, 1.7E+00, 5.2E−03; ESYT2, 1.7E+00, 1.4E−04; |
| | TMEM131, 1.7E+00, 2.2E−03; TMEM106B, 1.7E+00, 2.3E−05; KALRN, 1.7E+00, 2.6E−04; BCKDHB, 1.7E+00, 7.1E−03; FAM13A, 1.7E+00, 2.2E−02; LPP, |
| | 1.8E+00, 1.3E−02; LMX1A, 1.8E+00, 5.2E−09; WDSUB1, 1.8E+00, 2.6E−07; CNTNAP4, 1.8E+00, 2.9E−11; MAP2, 1.8E+00, 1.6E−03; UBE2E2, 1.8E+00, |
| | 8.4E−03; PARD3B, 1.8E+00, 4.9E−03; CDK19, 1.8E+00, 9.8E−04; STK3, 1.8E+00, 1.5E−03; NCKAP5, 1.8E+00, 7.7E−07; ZNF609, 1.9E+00, 1.6E−03; |
| | IMMP2L, 1.9E+00, 8.4E−03; MLLT3, 1.9E+00, 1.9E−02; LRRC4C, 1.9E+00, 2.9E−02; LOC729732, 1.9E+00, 2.1E−03; MAML3, 1.9E+00, 9.2E−04; ZZZ3, |
| | 1.9E+00, 2.5E−04; REPS1, 1.9E+00, 1.0E−04; C8orf34, 1.9E+00, 2.3E−08; FOCAD, 1.9E+00, 1.1E−03; PAPPA, 1.9E+00, 1.7E−08; GUCY1A1, 1.9E+00, 2.6E−13; |
| | TPD52L1, 1.9E+00, 1.2E−07; FREM2, 1.9E+00, 2.7E−05; ARID1B, 1.9E+00, 1.8E−03; EEA1, 1.9E+00, 5.2E−06; FZD3, 1.9E+00, 9.0E−05; MALAT1, |
| | 1.9E+00, 9.8E−17; STON2, 1.9E+00, 4.1E−05; GRHL2, 1.9E+00, 1.8E−04; GRIA3, 2.0E+00, 2.5E−10; FTX, 2.0E+00, 2.5E−04; PAX7, 2.0E+00, 5.0E−12; |
| | HMGA2, 2.0E+00, 2.7E−03; KMT2C, 2.0E+00, 1.6E−03; EXOC4, 2.0E+00, 9.7E−04; FER, 2.0E+00, 1.8E−04; WWOX, 2.0E+00, 5.6E−04; KCNJ3, 2.0E+00, |
| | 8.2E−10; MBD5, 2.1E+00, 5.9E−04; PTPN14, 2.1E+00, 1.7E−03; NEBL, 2.1E+00, 1.4E−04; LOC107986022, 2.1E+00, 2.1E−08; DST, 2.1E+00, 5.6E−05; |
| | HDAC8, 2.1E+00, 5.1E−06; ZBTB20, 2.1E+00, 3.0E−04; MSI2, 2.1E+00, 3.3E−04; SFMBT2, 2.1E+00, 4.3E−07; ELF1, 2.1E+00, 5.7E−06; SMYD3, 2.1E+00, |
| | 1.0E−03; STT3B, 2.1E+00, 1.7E−04; TTTY14, 2.1E+00, 8.2E−05; ESRP1, 2.2E+00, 2.3E−05; LINC01456, 2.2E+00, 7.9E−08; MID1, 2.2E+00, 2.0E−04; |
| | CCSER1, 2.2E+00, 2.0E−06; TANC1, 2.2E+00, 1.0E−05; NABP1, 2.2E+00, 1.4E−11; FBXL21P, 2.2E+00, 2.0E−17; TMEM108, 2.2E+00, 1.2E−06; PTPN13, |
| | 2.3E+00, 9.5E−05; PTPRK, 2.3E+00, 1.0E−04; NAALADL2, 2.3E+00, 4.9E−07; OLFM3, 2.3E+00, 8.2E−13; DNAJC15, 2.4E+00, 1.2E−07; PRKCH, 2.4E+00, |
| | 1.2E−15; LINC00673, 2.4E+00, 5.1E−08; NLGN1, 2.4E+00, 4.9E−04; C15orf41, 2.4E+00, 1.9E−07; LOC105379109, 2.4E+00, 3.1E−18; CDK13, 2.4E+00, |
| | 9.4E−06; BBS9, 2.4E+00, 2.8E−06; LOC100288798, 2.4E+00, 8.4E−09; ANK3, 2.4E+00, 1.0E−05; LOC101927182, 2.4E+00, 2.3E−18; NHS, 2.5E+00, 4.9E−07; |
| | LOC644919, 2.5E+00, 3.9E−06; PBX1, 2.5E+00, 3.7E−08; MAP3K20, 2.5E+00, 1.8E−13; ADGRL2, 2.5E+00, 5.2E−06; FHIT, 2.5E+00, 1.5E−07; |
| | ANKRD50, 2.5E+00, 2.8E−07; SGCD, 2.6E+00, 1.3E−20; MEIS2, 2.6E+00, 6.0E−12; PCDH11Y, 2.6E+00, 5.9E−09; TSHZ2, 2.6E+00, 5.6E−07; SEMA3C, |
| | 2.7E+00, 2.9E−13; OPHN1, 2.7E+00, 7.1E−11; UTRN, 2.7E+00, 1.4E−07; THRB, 2.7E+00, 4.6E−07; HS3ST3A1, 2.8E+00, 7.6E−13; LINC00278, 2.8E+00, |
| | 6.1E−25; MAPK10, 2.8E+00, 3.1E−10; SPAG16, 2.9E+00, 2.0E−11; RORA, 2.9E+00, 5.1E−07; CAST, 2.9E+00, 6.5E−10; NCOA7, 2.9E+00, 3.6E−16; CA10, |
| | 2.9E+00, 1.8E−12; CASC15, 3.0E+00, 9.4E−10; TMEFF2, 3.0E+00, 5.5E−20; TSPAN5, 3.1E+00, 3.2E−11; PKNOX2, 3.1E+00, 4.5E−13; FRAS1, 3.1E+00, 4.0E−10; |
| | CPNE8, 3.2E+00, 6.3E−12; ROBO2, 3.2E+00, 2.4E−06; SSBP2, 3.2E+00, 2.0E−09; CNTN4, 3.2E+00, 5.7E−09; ZC3H12B, 3.2E+00, 6.2E−10; KCNH7, |
| | 3.2E+00, 3.5E−17; ACSS3, 3.4E+00, 1.2E−15; PDE10A, 3.4E+00, 1.5E−11; EDIL3, 3.4E+00, 6.3E−12; THSD7A, 3.5E+00, 1.8E−12; VAV3, 3.5E+00, 3.0E−18; |
| | KIAA1217, 3.5E+00, 4.4E−13; PRSS23, 3.5E+00, 7.8E−19; PARP8, 3.6E+00, 3.5E−13; FREM1, 3.6E+00, 5.1E−12; SLC27A6, 3.7E+00, 5.1E−25; GREB1L, |
| | 3.7E+00, 1.3E−11; ASTN2, 3.7E+00, 1.3E−13; PTPRD, 3.7E+00, 4.5E−12; TANC2, 3.8E+00, 7.1E−17; PRKG1, 3.9E+00, 1.2E−12; NRXN3, 3.9E+00, 7.1E−11; |
| | DSP, 3.9E+00, 2.5E−13; CREB5, 3.9E+00, 1.2E−15; IL1RAPL2, 4.1E+00, 3.9E−18; PDE4D, 4.3E+00, 3.0E−13; DACH1, 4.3E+00, 3.8E−20; HAPLN1, 4.5E+00, |
| | 5.0E−21; ERBB4, 4.5E+00, 1.4E−18; LRRC7, 4.5E+00, 9.5E−28; ARL15, 4.6E+00, 4.4E−20; LOC284294, 4.7E+00, 1.6E−23; FBN2, 4.8E+00, 3.8E−18; |
| | ANKRD6, 5.1E+00, 2.3E−30; LOC107986324, 5.5E+00, 1.1E−33; ZNF385B, 5.7E+00, 6.7E−23; SORBS2, 5.8E+00, 2.3E−28; SLIT2, 6.4E+00, 2.3E−30; |
| | BMPER, 6.5E+00, 4.3E−51; MIR4300HG, 7.5E+00, 2.5E−65; LRP1B, 9.4E+00, 3.4E−62; HS6ST3, 1.1E+01, 2.2E−92 |
| 11-0 | LRRTM4, −2.0E+00, 1.4E−05; DSP, −2.0E+00, 6.4E−07; KRT18, −1.9E+00, 4.5E−08; TTN, −1.9E+00, 1.6E−03; LAMB1, −1.8E+00, 8.5E−07; CASC15, −1.8E+00, |
| | 4.2E−05; RBFOX1, −1.8E+00, 2.7E−04; ANK3, −1.7E+00, 1.2E−04; S100A10, −1.7E+00, 1.7E−06; KIAA1217, −1.6E+00, 1.6E−04; DST, −1.6E+00, 3.9E−04; |
| | SLC2A3, −1.6E+00, 3.8E−04; COL4A1, −1.5E+00, 3.8E−04; SPTBN1, −1.5E+00, 1.7E−03; HAS2, −1.4E+00, 1.5E−03; LOC339862, −1.4E+00, 5.1E−03; LAMA1, |
| | −1.4E+00, 9.1E−04; DLGAP1, −1.4E+00, 1.4E−03; SERPINE2, −1.4E+00, 3.0E−02; FAM184A, −1.4E+00, 3.7E−04; USO1, −1.4E+00, 3.0E−03; CHODL, −1.4E+00, |
| | 2.1E−04; NLGN4Y, −1.3E+00, 1.4E−02; CCDC141, −1.3E+00, 9.7E−03; RPS9, −1.3E+00, 7.4E−03; PATJ, −1.3E+00, 1.4E−02; KAZN, −1.3E+00, 1.6E−02; FRAS1, |
| | −1.3E+00, 1.5E−02; LOC107985961, −1.2E+00, 1.3E−04; NRCAM, −1.2E+00, 3.8E−04; CALM1, −1.2E+00, 2.8E−02; LOC105378798, −1.2E+00, 2.7E−07; |
| | ANXA2, −1.2E+00, 6.2E−03; RPS15, −1.2E+00, 2.8E−02; FGD6, −1.2E+00, 6.9E−03; FOXP1, −1.2E+00, 3.1E−02; FLNB, −1.2E+00, 1.6E−02; NSD1, −1.2E+00, |
| | 2.6E−02; SPARC, −1.2E+00, 8.6E−03; TET1, −1.2E+00, 4.2E−02; SERINC5, −1.2E+00, 2.6E−02; RBMS1, −1.2E+00, 4.5E−02; LOC100505817, −1.2E+00, 7.0E−05; |
| | VCAN, −1.2E+00, 1.8E−02; LOC107986777, −1.1E+00, 2.0E−02; ARHGAP42, −1.1E+00, 4.7E−02; PRICKLE2, −1.1E+00, 6.0E−03; LOC107986770, |
| | −1.1E+00, 2.3E−02; LOC728755, −1.1E+00, 3.1E−02; CADM2, −1.1E+00, 3.7E−02; DLGAP5, −1.1E+00, 2.8E−02; LOC105377901, −1.1E+00, 5.5E−05; PKP2, |
| | −1.1E+00, 1.7E−04; RBM47, −1.1E+00, 2.0E−02; ADGRA3, −1.1E+00, 4.6E−02; PRKCA, −1.0E+00, 3.4E−02; ZNF544, −1.0E+00, 4.1E−03; EPHA6, −1.0E+00, |
| | 1.0E−02; LOC107985962, −1.0E+00, 6.7E−03; DTWD2, −1.0E+00, 2.2E−03; KRT19, −1.0E+00, 1.4E−02; LOC107985037, −9.8E−01, 9.2E−03; RPS6, |
| | −9.8E−01, 1.7E−02; TPD52, −9.8E−01, 1.1E−02; MSN, −9.7E−01, 4.9E−02; LOC107985661, −9.6E−01, 3.4E−02; CD109, −9.6E−01, 3.4E−04; LOC102467213, |
| | −9.5E−01, 1.2E−02; RPL13A, −9.5E−01, 1.6E−02; ABHD12B, −9.4E−01, 2.3E−05; PCAT14, −9.0E−01, 1.7E−03; LAMA2, −8.8E−01, 4.4E−02; LRFN5, −8.6E−01, 1.3E−02; |
| | ITGB5, −8.4E−01, 4.6E−02; LOC105369876, −8.2E−01, 1.5E−02; IFITM1, −8.2E−01, 4.9E−02; LINC00428, −8.2E−01, 3.0E−02; LOC101928283, −8.1E−01, 7.2E−04; |
| | LINC00922, −8.1E−01, 3.1E−02; STOX2, −8.1E−01, 3.9E−03; TRMT9B, −8.1E−01, 1.4E−02; SLC2A1, −8.0E−01, 6.9E−04; OSBP, −8.0E−01, 2.6E−02; GRID2, |
| | −7.8E−01, 2.4E−04; CD177, −7.5E−01, 1.3E−03; ASAP3, −7.3E−01, 1.8E−02; SAMHD1, −7.2E−01, 3.1E−02; PPFIBP2, −7.2E−01, 3.5E−02; MRS2, −7.1E−01, 2.2E−02; |
| | ZNF611, −7.1E−01, 4.7E−02; BDNF-AS, −7.1E−01, 1.6E−02; COL6A3, −7.0E−01, 3.8E−02; ANXA1, −6.9E−01, 2.1E−03; LOC101927768, −6.9E−01, 2.8E−02; |
| | LYPD6B, −6.8E−01, 3.9E−03; SLC4A8, −6.8E−01, 1.8E−02; LINC00673, −6.8E−01, 6.7E−03; CPNE8, −6.7E−01, 3.4E−02; RHPN2, −6.7E−01, 2.9E−02; ZNF85, |
| | −6.6E−01, 4.0E−02; ATP6, −6.6E−01, 4.0E−02; LOC107987087, −6.6E−01, 2.2E−04; ST3GAL1, −6.5E−01, 1.7E−03; PCSK9, −6.5E−01, 2.8E−02; LINC00261, |
| | −6.5E−01, 2.2E−03; LOC105370826, −6.4E−01, 1.2E−03; AMOT, −6.3E−01, 3.5E−03; LCP1, −6.2E−01, 9.5E−03; ERRFI1, −6.1E−01, 4.1E−02; ANXA3, −6.1E−01, |
| | 1.0E−03; ASRGL1, −6.1E−01, 1.1E−02; MRPS7, −6.0E−01, 3.5E−02; LOC107985710, −6.0E−01, 3.1E−02; ND4, −5.9E−01, 7.4E−03; HPGD, −5.9E−01, 4.8E−03; |
| | CARD11, −5.8E−01, 1.1E−02; LAMC2, −5.8E−01, 1.1E−02; TEK, −5.6E−01, 4.0E−02; SYNPR, −5.6E−01, 3.2E−02; MFAP3L, −4.9E−01, 2.8E−02; WNT8A, |
| | −4.9E−01, 6.0E−03; F3, −4.7E−01, 2.5E−03; TBXT, −4.2E−01, 2.6E−02; LOC107984117, −4.2E−01, 2.3E−02; HNF4A, −4.1E−01, 1.5E−02; LOC107986620, −3.9E−01, |
| | 1.6E−02; LOC105372044, −3.4E−01, 2.1E−02; HBZ, 9.9E−02, 3.9E−02; LOC105372104, 1.1E−01, 3.2E−02; LRRC36, 1.2E−01, 3.4E−02; LOC102724055, |
| | 1.3E−01, 1.6E−02; LOC105377061, 1.4E−01, 2.3E−02; LOC286083, 1.6E−01, 3.6E−02; SFRP5, 1.7E−01, 7.2E−04; LOC105369463, 2.0E−01, 1.8E−02; |
| | LOC101927080, 2.1E−01, 1.7E−02; LTBP3, 2.2E−01, 4.1E−04; LOC107984120, 2.7E−01, 3.9E−02; STX11, 2.8E−01, 5.0E−03; TMEM186, 3.0E−01, 2.3E−03; |
| | LOC105371078, 3.0E−01, 3.4E−02; LOC105369464, 3.4E−01, 1.8E−04; JHY, 3.5E−01, 3.4E−03; HDC, 3.5E−01, 3.4E−04; FAM13C, 3.6E−01, 9.6E−03; |
| | LOC105379880, 3.7E−01, 1.9E−02; SLIT1, 4.0E−01, 1.4E−02; LINC00446, 4.0E−01, 2.0E−03; IFT22, 4.0E−01, 2.5E−02; ISG20, 4.1E−01, 4.4E−02; WNT9B, |
| | 4.2E−01, 2.6E−02; LOC105375138, 4.5E−01, 4.2E−02; LOC105369360, 4.7E−01, 4.9E−04; LOC105374464, 4.7E−01, 1.9E−03; PLG, 4.8E−01, 1.6E−03; |
| | LOC105374277, 5.0E−01, 2.5E−03; PLAGL1, 5.0E−01, 1.2E−02; LOC105375551, 5.0E−01, 3.5E−04; MAMLD1, 5.1E−01, 3.9E−03; LOC105374957, 5.2E−01, |
| | 2.8E−05; LOC107986134, 5.2E−01, 1.7E−03; SLC1A1, 5.3E−01, 4.4E−03; LOC107986178, 5.3E−01, 1.8E−04; GUCY1A1, 5.4E−01, 5.4E−03; LOC101926941, |
| | 5.6E−01, 4.1E−02; PI15, 5.7E−01, 1.9E−03; SLC4A10, 5.8E−01, 4.2E−05; KCNJ5, 6.0E−01, 1.7E−03; B3GAT2, 6.0E−01, 3.5E−05; LOC105372121, 6.0E−01, |
| | 1.5E−03; ADAM12, 6.0E−01, 4.1E−02; LOC105372763, 6.1E−01, 4.3E−06; SETD7, 6.2E−01, 3.8E−02; GABRB2, 6.2E−01, 3.1E−02; TBR1, 6.4E−01, 3.9E−06; |
| | ARHGAP20, 6.4E−01, 9.8E−07; LOC105373696, 6.5E−01, 4.1E−04; KIF24, 6.5E−01, 1.3E−03; WDR86, 6.6E−01, 1.8E−02; LOC101927359, 6.8E−01, 7.7E−04; |
| | AGPAT3, 6.8E−01, 2.8E−02; GATM, 6.9E−01, 5.9E−04; ACOX3, 6.9E−01, 3.2E−02; PCDH20, 6.9E−01, 2.7E−07; NAB1, 7.0E−01, 2.9E−02; LINC00907, 7.1E−01, |
| | 7.4E−03; LOC105374211, 7.2E−01, 1.3E−04; LOC105374276, 7.2E−01, 6.4E−08; TEX9, 7.2E−01, 4.6E−02; LINC01592, 7.2E−01, 2.8E−05; LOC105374145, |
| | 7.2E−01, 3.9E−06; FSTL5, 7.2E−01, 2.1E−03; TRG-AS1, 7.3E−01, 2.7E−03; NDST4, 7.3E−01, 1.9E−04; LOC105374312, 7.4E−01, 3.3E−03; CLDN11, 7.4E−01, |
| | 1.0E−04; EOGT, 7.4E−01, 1.2E−03; STK17A, 7.5E−01, 8.1E−04; EEFSEC, 7.6E−01, 4.6E−02; SYT9, 7.6E−01, 9.6E−05; FBN1, 7.7E−01, 1.9E−02; LOC102723471, |
| | 7.7E−01, 2.1E−04; DDR2, 7.7E−01, 2.7E−07; TSPAN12, 7.9E−01, 4.8E−03; LYST, 7.9E−01, 1.6E−04; EPS8, 8.0E−01, 7.9E−03; ANGPT1, 8.0E−01, 6.0E−03; |
| | COL24A1, 8.0E−01, 3.8E−06; BMPR1B, 8.1E−01, 1.4E−02; HIVEP2, 8.3E−01, 4.9E−02; GNG12-AS1, 8.3E−01, 3.7E−04; TRPC3, 8.4E−01, 1.3E−06; CSMD3, |
| | 8.6E−01, 3.7E−03; ST8SIA1, 8.7E−01, 3.9E−04; FAM189A2, 8.8E−01, 2.2E−04; NR5A2, 8.9E−01, 1.9E−07; TPK1, 8.9E−01, 1.9E−02; HDAC9, 9.1E−01, 4.6E−02; |
| | LOC105374189, 9.1E−01, 2.8E−05; DSEL, 9.2E−01, 2.4E−04; DGKB, 9.4E−01, 3.8E−02; MIR4500HG, 9.6E−01, 1.3E−07; DKK1, 9.6E−01, 3.3E−06; |
| | TGFBR3, 9.8E−01, 9.4E−03; DENND1B, 1.0E+00, 6.0E−03; LINC00113, 1.0E+00, 3.2E−08; CFLAR, 1.0E+00, 1.5E−03; LOC101927708, 1.0E+00, 3.9E−03; |
| | LRRCC1, 1.0E+00, 2.5E−04; ATAD2, 1.0E+00, 3.6E−02; FGF10, 1.0E+00, 2.1E−08; TENM2, 1.1E+00, 6.9E−03; LOC105370507, 1.1E+00, 8.8E−08; SDK2, |
| | 1.1E+00, 3.0E−02; VAV3, 1.1E+00, 9.4E−03; SMAD6, 1.1E+00, 6.6E−03; AEN, 1.1E+00, 2.3E−04; ST6GALNAC5, 1.1E+00, 9.2E−03; GLIS3, 1.1E+00, 3.8E−05; |
| | LOC105369250, 1.1E+00, 1.2E−03; ACVR2A, 1.1E+00, 2.0E−03; LOC105370610, 1.1E+00, 1.9E−03; KCNH7, 1.1E+00, 2.6E−06; CHL1, 1.1E+00, 2.7E−09; |
| | CLMP, 1.1E+00, 5.8E−04; CHST9, 1.1E+00, 2.9E−02; LINC00458, 1.1E+00, 1.1E−02; SLC9A9, 1.1E+00, 3.2E−04; CAP2, 1.1E+00, 1.3E−03; |
| | LOC107986100, 1.2E+00, 3.2E−04; SACM1L, 1.2E+00, 3.3E−03; KIF5C, 1.2E+00, 1.8E−02; PREX1, 1.2E+00, 1.2E−04; NBEA, 1.2E+00, 3.5E−02; FMN2, |
| | 1.2E+00, 3.4E−04; COL13A1, 1.2E+00, 8.8E−04; EFNA5, 1.2E+00, 4.8E−02; NTN1, 1.2E+00, 1.3E−03; NSD2, 1.2E+00, 1.9E−02; BMPER, 1.2E+00, 5.0E−02; |
| | MDK, 1.2E+00, 3.5E−02; DOK6, 1.2E+00, 1.2E−03; PTPRM, 1.3E+00, 2.3E−02; COL2A1, 1.3E+00, 3.2E−06; FLRT2, 1.3E+00, 6.3E−03; HEG1, 1.3E+00, 2.7E−04; |
| | PKM, 1.3E+00, 3.6E−02; AUTS2, 1.3E+00, 1.5E−03; ST6GALNAC3, 1.3E+00, 2.9E−02; COL6A4P2, 1.3E+00, 6.1E−12; SULF1, 1.3E+00, 2.2E−06; ERICH1- |
| | AS1, 1.3E+00, 6.6E−05; LOC107986021, 1.3E+00, 1.2E−04; LRIG3, 1.3E+00, 3.4E−03; NCAM1, 1.3E+00, 1.1E−04; LOC105379219, 1.3E+00, 4.4E−03; |
| | LARGE, 1.3E+00, 1.2E−02; JAKMIP1, 1.3E+00, 3.0E−07; SAMD5, 1.3E+00, 8.7E−06; DGKI, 1.4E+00, 3.9E−06; GPC6, 1.4E+00, 2.6E−02; NKAIN3, 1.4E+00, |
| | 6.1E−04; COBLL1, 1.4E+00, 1.0E−04; PARD3B, 1.4E+00, 5.2E−03; TMTC1, 1.4E+00, 4.4E−03; THSD7A, 1.4E+00, 4.9E−04; HCN1, 1.4E+00, 6.8E−04; |
| | MAPK10, 1.4E+00, 6.0E−03; MEIS2, 1.4E+00, 9.8E−06; RBM20, 1.4E+00, 2.1E−07; SYNDIG1, 1.5E+00, 5.3E−04; DET1, 1.5E+00, 2.8E−09; NRG3, 1.5E+00, |
| | 7.6E−03; KCNJ3, 1.5E+00, 3.2E−06; SLIT2, 1.5E+00, 2.0E−02; LOC401478, 1.5E+00, 1.3E−14; RN7SK, 1.5E+00, 8.1E−03; LOC101928217, 1.5E+00, 3.8E−05; |
| | LDLRAD4, 1.5E+00, 1.8E−05; CNTN4, 1.5E+00, 1.4E−04; TMEM232, 1.5E+00, 1.5E−05; ITGB1, 1.5E+00, 4.2E−04; DNAJC24, 1.5E+00, 1.6E−06; TAFA2, |
| | 1.5E+00, 5.8E−08; PLSCR1, 1.5E+00, 2.3E−07; RASGRP1, 1.6E+00, 4.4E−10; GPC5, 1.6E+00, 7.8E−10; MCF2L2, 1.6E+00, 2.8E−05; ITPR1, 1.6E+00, |
| | 1.5E−08; CCSER1, 1.6E+00, 2.0E−06; ARHGAP40, 1.6E+00, 6.1E−18; LOC101927056, 1.6E+00, 1.9E−08; CACNA2D1, 1.6E+00, 8.0E−04; LRP1B, 1.6E+00, 4.6E−07; |
| | C1GALT1, 1.6E+00, 8.5E−05; DIAPH2, 1.6E+00, 1.9E−04; PDGFD, 1.7E+00, 2.6E−08; THSD7B, 1.7E+00, 5.1E−13; ADCY2, 1.7E+00, 8.5E−04; |
| | LINC01586, 1.7E+00, 1.8E−17; PTPRD, 1.7E+00, 1.0E−04; LDB2, 1.7E+00, 2.7E−04; EPB41L3, 1.8E+00, 2.0E−08; PLSCR2, 1.8E+00, 6.5E−18; NTN4, |
| | 1.8E+00, 1.6E−08; LOC105369715, 1.8E+00, 1.9E−18; LHX1, 1.8E+00, 1.8E−09; LOC107986324, 1.9E+00, 3.2E−11; ZNF521, 1.9E+00, 6.1E−06; NEO1, |
| | 1.9E+00, 6.9E−06; RNF219-AS1, 1.9E+00, 5.7E−11; TNIK, 1.9E+00, 4.6E−07; PALLD, 2.0E+00, 8.5E−07; HS3ST3A1, 2.0E+00, 3.3E−07; HTR1E, 2.0E+00, |
| | 4.7E−19; VWC2L, 2.0E+00, 2.2E−07; NCOA1, 2.0E+00, 9.0E−08; TENM4, 2.1E+00, 3.0E−06; FAT4, 2.1E+00, 3.5E−11; TMTC2, 2.1E+00, 8.8E−06; PRTG, |
| | 2.1E+00, 3.1E−06; ANK2, 2.2E+00, 2.8E−12; PCDH10, 2.2E+00, 1.6E−10; SLC8A1, 2.3E+00, 4.6E−18; LOC101927973, 2.3E+00, 8.8E−15; COL5A2, 2.3E+00, |
| | 1.5E−07; SLC5A4, 2.4E+00, 2.1E−15; P3H2, 2.4E+00, 8.8E−08; CNTNAP2, 2.4E+00, 1.3E−09; LOC100506990, 2.5E+00, 2.4E−11; CHST11, 2.6E+00, 3.6E−12; |
| | FBN2, 2.6E+00, 6.3E−09; CDH18, 2.7E+00, 7.2E−18; KCNQ5, 2.7E+00, 7.5E−17; DACH1, 2.8E+00, 2.3E−16; PCDH7, 2.8E+00, 3.6E−13; LOC107985819, |
| | 2.8E+00, 4.0E−27; WLS, 2.8E+00, 2.2E−16; PLCB1, 2.8E+00, 1.3E−12; LOC729732, 2.8E+00, 5.3E−13; CCND2, 2.9E+00, 1.0E−13; SPAG16, 3.0E+00, 6.5E−11; |
| | TRABD2B, 3.0E+00, 9.1E−17; CNTNAP5, 3.0E+00, 2.4E−22; RELN, 3.1E+00, 1.2E−33; LOC284825, 3.1E+00, 9.6E−20; PRKD1, 3.2E+00, 1.7E−17; SOX5, |
| | 3.2E+00, 5.1E−15; ERBB4, 3.2E+00, 3.1E−13; ALPK2, 3.3E+00, 1.3E−18; LOC102724210, 3.3E+00, 1.7E−14; SHISA6, 3.3E+00, 1.7E−17; SAMD3, 3.3E+00, |
| | 1.9E−21; PLSCR4, 3.3E+00, 2.5E−32; DYNC111, 3.7E+00, 9.4E−32; ADAMTS3, 3.7E+00, 2.6E−29; RHOBTB3, 3.8E+00, 3.2E−29; ESRRG, 3.9E+00, 6.0E−24; |
| | DCC, 4.0E+00, 1.1E−28; GRIK2, 4.3E+00, 6.8E−36; ZFPM2, 4.3E+00, 4.9E−39; EPHA3, 4.5E+00, 3.2E−45; LOC105370302, 5.2E+00, 3.5E−48; DSCAM, |
| | 5.3E+00, 7.6E−44; LOC102724419, 6.5E+00, 2.5E−48 |
| 11-1 | LOC105373347, 1.4E+00, 6.6E−03; LOC107986980, 1.6E+00, 1.9E−02; HSPA12A, 1.7E+00, 1.3E−02; LOC107986324, 1.7E+00, 4.7E−02; LOC105377861, |
| | 1.7E+00, 3.6E−02; HMX1, 1.7E+00, 6.6E−03; LOC284825, 1.8E+00, 2.4E−02; LOC102724542, 1.8E+00, 1.5E−03; FAM110B, 1.8E+00, 3.1E−02; CYP11A1, |
| | 1.8E+00, 3.1E−02; LOC105374029, 1.9E+00, 8.4E−03; LOC105374474, 1.9E+00, 4.5E−04; KCNJ3, 2.0E+00, 6.6E−03; MRC2, 2.1E+00, 1.4E−02; |
| | LINC00458, 2.1E+00, 4.8E−02; LOC107986602, 2.1E+00, 3.1E−02; LOC101929534, 2.2E+00, 2.9E−04; ST6GALNAC5, 2.2E+00, 2.7E−02; FYN, 2.2E+00, |
| | 3.5E−02; PGM5P2, 2.3E+00, 4.9E−03; TMEM232, 2.3E+00, 6.6E−03; LOC101927973, 2.4E+00, 1.4E−04; KIAA1109, 2.4E+00, 6.6E−03; USH2A, 2.5E+00, |
| | 1.2E−04; LOC105377860, 2.5E+00, 1.4E−02; CDH4, 2.6E+00, 4.2E−02; PLCL1, 2.7E+00, 6.6E−03; DTWD2, 2.7E+00, 4.6E−03; PGM5, 2.7E+00, 7.3E−04; |
| | ZC3H12B, 2.7E+00, 4.1E−02; LOC105374220, 2.8E+00, 6.2E−11; LDB2, 2.8E+00, 2.3E−02; LHFP, 2.8E+00, 2.3E−02; LOC105369436, 2.9E+00, 3.8E−05; |
| | AFF3, 3.1E+00, 1.7E−03; SERPINE2, 3.1E+00, 4.9E−04; LOC730100, 3.3E+00, 3.7E−06; ADCY2, 3.7E+00, 1.3E−04; SPATA16, 3.7E+00, 6.2E−11; SSBP2, |
| | 3.8E+00, 1.1E−05; IL1RAPL1, 3.9E+00, 1.1E−05; NLGN1, 4.2E+00, 4.2E−06; LOC105369512, 4.4E+00, 4.1E−07; LOC107986021, 6.1E+00, 8.8E−13 |
| 12-0 | ESRG, −1.4E+00, 2.1E−02; HS3ST4, −1.2E+00, 3.2E−02; GRID2, −1.0E+00, 1.6E−03; IFITM1, −8.8E−01, 3.8E−02; PTMA, −8.3E−01, 4.9E−02; TMPRSS11E, |
| | −2.5E−01, 4.2E−02; EOMES, −2.4E−01, 1.1E−02; CABP1, −1.9E−01, 5.4E−03; ABCD1, 2.4E−01, 4.3E−02; ITGA10, 3.8E−01, 2.5E−02; FOSL1, 3.9E−01, 1.4E−02; |
| | LOC100507002, 4.1E−01, 3.5E−02; LOC105374596, 4.3E−01, 2.8E−03; LOC105374459, 4.3E−01, 2.9E−03; ZNF503, 4.5E−01, 2.1E−02; RBM24, 4.8E−01, |
| | 4.1E−03; LINC01487, 4.8E−01, 1.7E−03; OLFML2A, 4.9E−01, 3.6E−02; ATP2B2, 4.9E−01, 3.9E−02; RFTN1, 5.5E−01, 3.2E−02; MID2, 6.0E−01, 3.1E−03; |
| | HS3ST1, 6.0E−01, 1.9E−02; PNP, 6.1E−01, 6.0E−03; ARHGAP40, 6.2E−01, 1.4E−02; VWCE, 6.4E−01, 5.9E−03; EMP3, 6.7E−01, 9.4E−03; FKBP14, 6.7E−01, |
| | 3.6E−02; MALAT1, 6.7E−01, 2.0E−02; RGS9, 6.9E−01, 2.3E−02; LOC100506718, 7.0E−01, 6.7E−03; IER3, 7.1E−01, 1.0E−02; PPP1R14C, 7.2E−01, 4.5E−04; |
| | NDFIP2, 7.3E−01, 2.5E−02; FOXA1, 7.3E−01, 7.0E−04; RASA3, 7.5E−01, 2.3E−02; LINC01320, 7.5E−01, 6.5E−04; LOC100996643, 7.6E−01, 1.4E−02; RUSC2, |
| | 7.7E−01, 9.5E−03; NIBAN2, 7.7E−01, 4.4E−02; IMPAD1, 7.8E−01, 2.0E−02; NPFFR2, 7.8E−01, 3.0E−03; PLXND1, 7.9E−01, 6.9E−03; DCTN4, 7.9E−01, 3.6E−02; |
| | DENND3, 7.9E−01, 2.0E−03; TBXT, 8.0E−01, 3.6E−02; ACAN, 8.1E−01, 1.2E−04; OGFRL1, 8.1E−01, 2.7E−02; SRGAP2, 8.2E−01, 2.7E−02; MBNL2, 8.3E−01, |
| | 5.0E−02; PRSS23, 8.3E−01, 1.9E−02; ADAMTS17, 8.7E−01, 4.1E−02; KCTD20, 8.7E−01, 4.9E−02; UBA3, 8.7E−01, 3.0E−02; RHOJ, 8.8E−01, 2.7E−05; |
| | LZTS1, 8.8E−01, 2.1E−02; SNAPC3, 8.8E−01, 4.2E−02; RNF144B, 8.9E−01, 2.8E−03; DCC, 9.0E−01, 3.7E−02; LRATD2, 9.0E−01, 1.2E−02; ADCY8, 9.1E−01, |
| | 3.0E−02; MCAM, 9.1E−01, 1.5E−03; ASXL1, 9.2E−01, 1.8E−02; GALNT11, 9.3E−01, 2.7E−02; EFEMP1, 9.3E−01, 9.4E−03; CDH10, 9.3E−01, 4.3E−02; |
| | FAM114A1, 9.3E−01, 1.3E−02; GNG12, 9.4E−01, 1.8E−02; NOD1, 9.4E−01, 1.6E−03; PMP22, 9.4E−01, 4.9E−02; PPP1R3C, 9.7E−01, 1.2E−03; RBM20, 9.9E−01, |
| | 2.7E−02; BAZ1A, 1.0E+00, 3.6E−02; TMC03, 1.0E+00, 9.1E−03; LOC105374693, 1.0E+00, 1.2E−02; SLC20A1, 1.0E+00, 5.0E−02; NEK6, 1.0E+00, 9.5E−03; |
| | CPQ, 1.0E+00, 6.1E−04; KDELR2, 1.0E+00, 4.6E−02; CDC27, 1.0E+00, 4.1E−02; RNF19A, 1.0E+00, 4.7E−03; DDR2, 1.1E+00, 2.3E−04; SERPINE1, |
| | 1.1E+00, 7.6E−05; ACTB, 1.1E+00, 1.4E−02; ENTPD4, 1.1E+00, 1.7E−02; LOC105373997, 1.1E+00, 1.3E−02; CEP112, 1.1E+00, 1.1E−02; KCTD16, |
| | 1.1E+00, 2.3E−03; THBS1, 1.1E+00, 2.1E−02; CDH13, 1.1E+00, 1.0E−02; NOX4, 1.1E+00, 2.2E−05; NFIA, 1.1E+00, 3.7E−03; FLNC, 1.1E+00, 3.7E−03; |
| | SMURF2, 1.1E+00, 9.6E−03; ATP6V1B2, 1.1E+00, 2.4E−03; PPA1, 1.1E+00, 1.5E−02; RFTN2, 1.1E+00, 1.1E−02; NRP2, 1.1E+00, 1.6E−02; PAPSS1, |
| | 1.1E+00, 2.8E−02; BIRC2, 1.1E+00, 1.1E−03; MSRB3, 1.1E+00, 1.3E−04; DLC1, 1.1E+00, 1.1E−03; ELK3, 1.1E+00, 5.3E−03; DUSP6, 1.1E+00, 4.4E−03; |
| | FRMD6, 1.2E+00, 1.6E−03; GLIS3, 1.2E+00, 7.9E−03; EPSTI1, 1.2E+00, 1.1E−04; DLG5, 1.2E+00, 2.6E−02; TMEM178A, 1.2E+00, 6.2E−05; IGFBP7, |
| | 1.2E+00, 1.6E−04; SESTD1, 1.2E+00, 1.3E−02; DENND5A, 1.2E+00, 8.0E−03; HOOK3, 1.2E+00, 2.6E−02; PPM1H, 1.2E+00, 3.5E−02; ADAMTS6, 1.2E+00, |
| | 3.6E−02; LINC00673, 1.2E+00, 3.4E−03; SSH2, 1.2E+00, 1.9E−02; RARS1, 1.2E+00, 3.4E−02; MIR99AHG, 1.2E+00, 7.9E−03; CAPZA2, 1.2E+00, 1.1E−02; |
| | TRIM44, 1.2E+00, 7.9E−03; LOC284825, 1.2E+00, 1.5E−02; ABCG2, 1.2E+00, 7.9E−03; CA3, 1.2E+00, 2.3E−03; SSH1, 1.2E+00, 9.5E−04; AKAP11, 1.2E+00, |
| | 1.2E−02; KLF7, 1.3E+00, 3.6E−03; PLOD2, 1.3E+00, 2.4E−03; COL8A1, 1.3E+00, 1.0E−03; CYP11A1, 1.3E+00, 2.3E−03; SERTAD2, 1.3E+00, 8.1E−05; |
| | EPHB1, 1.3E+00, 2.3E−03; PSME4, 1.3E+00, 2.3E−02; FAM135B, 1.3E+00, 1.2E−03; SDC2, 1.3E+00, 4.2E−02; EIF2S1, 1.3E+00, 1.3E−03; GAS6, 1.3E+00, |
| | 2.2E−04; FDPS, 1.3E+00, 1.2E−02; EFR3A, 1.3E+00, 1.4E−02; RBMS2, 1.3E+00, 2.8E−04; COPG2, 1.3E+00, 2.6E−02; RNF219-AS1, 1.3E+00, 4.5E−04; |
| | LRMDA, 1.3E+00, 1.1E−03; LOC101927973, 1.3E+00, 4.7E−04; PCNX4, 1.3E+00, 1.4E−02; SPATA16, 1.3E+00, 2.3E−02; CCSER2, 1.3E+00, 1.1E−02; SENP6, |
| | 1.3E+00, 3.4E−02; EGF, 1.3E+00, 8.8E−04; PPFIA1, 1.4E+00, 7.6E−03; SOX9, 1.4E+00, 3.2E−05; CUL1, 1.4E+00, 3.8E−03; MYL6, 1.4E+00, 4.2E−02; |
| | LOC105377860, 1.4E+00, 7.7E−03; TLN1, 1.4E+00, 2.5E−03; DSTN, 1.4E+00, 2.7E−02; FTO, 1.4E+00, 6.0E−03; COPB2, 1.4E+00, 4.5E−03; VCL, 1.4E+00, |
| | 1.8E−02; PLXNA2, 1.4E+00, 8.3E−05; DICER1, 1.4E+00, 5.7E−03; AMOTL1, 1.4E+00, 2.7E−03; NFE2L2, 1.4E+00, 1.6E−05; KRT8, 1.5E+00, 7.1E−03; CAPN2, |
| | 1.5E+00, 2.4E−04; BCAT1, 1.5E+00, 9.5E−03; CDH11, 1.5E+00, 3.3E−03; PPFIBP1, 1.5E+00, 1.4E−03; LOXL2, 1.5E+00, 1.4E−05; TTC6, 1.5E+00, 9.9E−06; |
| | SEPTIN2, 1.5E+00, 9.1E−03; WWP2, 1.5E+00, 9.5E−05; PARVA, 1.6E+00, 1.9E−05; CDK6, 1.6E+00, 3.4E−03; ACSL3, 1.6E+00, 4.1E−03; NLGN4X, 1.6E+00, |
| | 1.8E−02; LIMCH1, 1.6E+00, 2.5E−03; GPC6, 1.6E+00, 1.7E−02; DNAJC15, 1.6E+00, 2.8E−06; GLIPR1, 1.6E+00, 4.1E−07; SPTAN1, 1.6E+00, 3.0E−03; |
| | ACTN4, 1.6E+00, 4.5E−03; COL4A1, 1.6E+00, 1.2E−03; MAP4, 1.6E+00, 4.1E−03; TRIO, 1.6E+00, 8.0E−03; DTNA, 1.6E+00, 4.0E−03; LRRFIP1, 1.6E+00, |
| | 3.8E−03; ZNF385B, 1.6E+00, 4.8E−04; SHROOM3, 1.6E+00, 6.5E−04; LDHA, 1.6E+00, 1.6E−03; SOX5, 1.6E+00, 1.1E−02; CCDC85A, 1.6E+00, 9.2E−07; |
| | PEG10, 1.6E+00, 6.1E−03; AP2B1, 1.6E+00, 8.2E−04; MYL9, 1.7E+00, 1.1E−04; RHOBTB3, 1.7E+00, 1.2E−06; TMEM232, 1.7E+00, 2.4E−04; MALT1, |
| | 1.7E+00, 1.4E−04; ZNF385D, 1.7E+00, 7.5E−05; DPYSL2, 1.7E+00, 2.0E−03; LIMS1, 1.7E+00, 6.5E−04; BACH2, 1.7E+00, 1.3E−05; SEMA3C, 1.7E+00, 9.2E−05; |
| | SOX6, 1.7E+00, 2.0E−06; DST, 1.7E+00, 2.8E−04; SPATS2L, 1.7E+00, 1.5E−03; TENM2, 1.7E+00, 5.6E−04; ARID3B, 1.8E+00, 8.3E−05; LOC102724210, |
| | 1.8E+00, 2.3E−04; EPB41L2, 1.8E+00, 5.0E−04; CRIM1, 1.8E+00, 2.4E−04; ATRNL1, 1.8E+00, 2.7E−04; CCN2, 1.8E+00, 1.5E−04; FSTL5, 1.8E+00, 2.5E−07; |
| | TPM4, 1.8E+00, 6.9E−04; NES, 1.8E+00, 2.4E−04; LAMB1, 1.8E+00, 1.4E−04; PALLD, 1.8E+00, 3.0E−04; TGFBR3, 1.9E+00, 9.7E−06; SLC2A1, 1.9E+00, |
| | 5.3E−06; FSTL1, 1.9E+00, 2.6E−04; COLGALT2, 1.9E+00, 4.2E−07; LOC102724419, 1.9E+00, 7.0E−07; EXT1, 1.9E+00, 3.0E−03; ACTN1, 1.9E+00, 9.1E−05; |
| | NQ01, 1.9E+00, 1.1E−06; ADARB1, 1.9E+00, 4.2E−07; CAP2, 1.9E+00, 7.4E−06; TRAM2, 1.9E+00, 2.2E−08; ZSWIM6, 1.9E+00, 9.3E−05; RUNX2, 1.9E+00, |
| | 8.9E−09; RNF217, 1.9E+00, 2.7E−06; MIR4435-2HG, 2.0E+00, 8.1E−07; NEAT1, 2.0E+00, 6.8E−04; TNS3, 2.0E+00, 6.6E−05; KRT19, 2.0E+00, 2.7E−05; |
| | GNB4, 2.0E+00, 1.7E−05; LPP, 2.1E+00, 8.5E−06; APP, 2.1E+00, 2.9E−06; TNFRSF21, 2.1E+00, 4.7E−07; USH2A, 2.1E+00, 2.8E−08; CHRM2, 2.1E+00, 2.7E−11; |
| | WFDC3, 2.1E+00, 7.8E−09; ADAM19, 2.1E+00, 1.3E−07; LINC00458, 2.2E+00, 2.1E−04; GMPR, 2.2E+00, 5.0E−06; STXBP6, 2.2E+00, 4.7E−08; CAV1, |
| | 2.2E+00, 8.8E−11; CYR61, 2.2E+00, 7.8E−09; ST3GAL1, 2.2E+00, 2.0E−10; MAML3, 2.3E+00, 3.5E−05; CD109, 2.3E+00, 1.5E−09; MYO3A, 2.3E+00, 4.8E−08; |
| | ABLIM1, 2.3E+00, 1.9E−07; EDNRB, 2.3E+00, 2.1E−08; CALD1, 2.4E+00, 4.2E−08; AKT3, 2.4E+00, 6.7E−07; MYH9, 2.4E+00, 2.0E−07; SHH, 2.4E+00, |
| | 2.7E−11; COL5A2, 2.4E+00, 2.0E−06; NEK7, 2.4E+00, 5.1E−08; NET1, 2.4E+00, 1.6E−08; RBMS3, 2.5E+00, 7.1E−09; NEDD9, 2.5E+00, 2.9E−12; |
| | LOC107986021, 2.5E+00, 3.3E−07; ST6GALNAC5, 2.5E+00, 1.5E−06; ANXA2, 2.5E+00, 6.8E−08; TPM1, 2.5E+00, 6.1E−08; CNTNAP2, 2.5E+00, 1.6E−08; |
| | DDAH1, 2.5E+00, 1.5E−10; SAMD4A, 2.6E+00, 2.5E−07; ASAP2, 2.6E+00, 9.5E−11; MB21D2, 2.6E+00, 1.0E−08; DTWD2, 2.6E+00, 1.3E−10; KHDRBS3, |
| | 2.6E+00, 1.5E−09; ADARB2, 2.6E+00, 1.1E−08; TANC1, 2.7E+00, 1.5E−11; ANXA1, 2.7E+00, 7.2E−10; AHNAK, 2.7E+00, 4.3E−08; MAML2, 2.7E+00, |
| | 1.4E−07; SEPTIN11, 2.8E+00, 2.6E−09; MEST, 2.8E+00, 5.8E−09; AKAP12, 3.0E+00, 1.5E−11; TAGLN, 3.1E+00, 1.2E−10; PTPRD, 3.2E+00, 1.4E−11; FLRT2, |
| | 3.2E+00, 1.9E−11; VIM, 3.2E+00, 3.2E−12; ANKRD1, 3.3E+00, 1.2E−08; ALPK2, 3.3E+00, 9.1E−19; MYOF, 3.4E+00, 1.1E−14; ITGB8, 3.5E+00, 5.4E−16; |
| | CCDC141, 3.5E+00, 7.1E−12; TNC, 3.7E+00, 6.9E−15; TTN, 3.7E+00, 3.0E−12; TRPS1, 3.8E+00, 5.5E−17; SERPINE2, 4.2E+00, 3.3E−19; ALCAM, 4.4E+00, |
| | 1.1E−14; COL11A1, 4.5E+00, 8.4E−21; SORBS2, 4.7E+00, 1.0E−21; SPP1, 5.0E+00, 9.5E−20; SLIT2, 5.1E+00, 4.4E−23; COL2A1, 6.6E+00, 9.7E−41; FN1, |
| | 7.1E+00, 5.5E−39 |
| 12-1 | CCDC141, 3.9E+00, 4.3E−06; TTN, 3.9E+00, 1.5E−05; CDH13, 3.7E+00, 4.6E−05; ZNF385D, 3.2E+00, 3.1E−05; LOC102724419, 2.9E+00, 4.6E−05; |
| | SPATA16, 2.7E+00, 6.8E−04; ASAP2, 2.3E+00, 5.9E−03; HAPLN1, 2.2E+00, 1.1E−02; FAM19A1, 2.2E+00, 4.6E−03; CDH10, 1.9E+00, 6.2E−03; COLEC12, |
| | 1.6E+00, 4.6E−02; CCL26, 1.5E+00, 4.5E−02; CYP7B1, 1.3E+00, 1.1E−02; SLC2A13, −1.4E+00, 4.7E−02 |
| 13 | MDK, −1.5E+01, 9.2E−03; CALM2, −1.4E+01, 2.1E−02; H4C5, −1.4E+01, 4.9E−02; SOX2, −1.3E+01, 3.8E−02; RPL30, −1.3E+01, 4.4E−02; CCNB1, −1.3E+01, |
| | 2.6E−02; PTMA, −1.2E+01, 7.6E−04; HMGB3, −1.2E+01, 4.9E−02; HNRNPU, −1.2E+01, 4.4E−02; GAPDH, −1.2E+01, 7.4E−03; PABPC1, −1.2E+01, 4.5E−02; |
| | TBX15, −2.4E+00, 3.8E−02; GMPPA, −2.3E+00, 4.0E−02; DSCR8, 5.4E−01, 4.5E−02; HSPB2, 5.4E−01, 4.5E−02; KCNA5, 5.4E−01, 4.5E−02; LOC101928080, |
| | 5.4E−01, 4.5E−02; LOC101929278, 5.4E−01, 4.5E−02; LOC105373019, 5.4E−01, 4.5E−02; LOC105373408, 5.4E−01, 4.5E−02; LOC107985740, 5.4E−01, |
| | 4.5E−02; LOC107986157, 5.4E−01, 4.5E−02; LOC107986453, 5.4E−01, 4.5E−02; LOC107986826, 5.4E−01, 4.5E−02; NUTM2D, 5.4E−01, 4.5E−02; RAET1K, |
| | 5.4E−01, 4.5E−02; RGS1, 5.4E−01, 4.5E−02; WNT11, 5.4E−01, 4.5E−02; BPIFB1, 6.3E−01, 3.8E−02; CD302, 6.3E−01, 3.8E−02; CLCNKB, 6.3E−01, 3.8E−02; |
| | CYP24A1, 6.3E−01, 3.8E−02; EMX1, 6.3E−01, 3.8E−02; LINC00502, 6.3E−01, 3.8E−02; LINC01330, 6.3E−01, 3.8E−02; LOC101927557, 6.3E−01, 3.8E−02; |
| | LOC102724497, 6.3E−01, 3.8E−02; LOC102724584, 6.3E−01, 3.8E−02; LOC105371091, 6.3E−01, 3.8E−02; LOC105375183, 6.3E−01, 3.8E−02; |
| | LOC107984352, 6.3E−01, 3.8E−02; MIR7112, 6.3E−01, 3.8E−02; MS4A5, 6.3E−01, 3.8E−02; NOX1, 6.3E−01, 3.8E−02; LOC101929974, 6.3E−01, 4.0E−02; |
| | LOC105369455, 6.3E−01, 4.0E−02; LOC105370443, 6.3E−01, 4.0E−02; LOC105371335, 6.3E−01, 4.0E−02; LOC105374596, 6.3E−01, 4.0E−02; |
| | LOC105374774, 6.3E−01, 4.0E−02; LOC105377324, 6.3E−01, 4.0E−02; LOC105377401, 6.3E−01, 4.0E−02; LOC105378651, 6.3E−01, 4.0E−02; |
| | LOC107985564, 6.3E−01, 4.0E−02; LZTS1-AS1, 6.3E−01, 4.0E−02; PRR18, 6.3E−01, 4.0E−02; SLC6A1, 6.3E−01, 4.0E−02; SPATA22, 6.3E−01, 4.0E−02; |
| | ZCCHC12, 6.3E−01, 4.0E−02; LINC01201, 7.3E−01, 4.4E−02; LOC105377411, 7.3E−01, 4.4E−02; LOC107984205, 7.3E−01, 4.4E−02; LOC107986635, 7.3E−01, |
| | 4.4E−02; MIR3654, 7.3E−01, 4.4E−02; SLC14A1, 7.3E−01, 4.4E−02; BPIFA3, 8.6E−01, 1.7E−02; CPLX4, 8.6E−01, 1.7E−02; FLJ36777, 8.6E−01, 1.7E−02; |
| | IFIH1, 8.6E−01, 1.7E−02; LOC105372647, 8.6E−01, 1.7E−02; LOC105374793, 8.6E−01, 1.7E−02; LOC107986651, 8.6E−01, 1.7E−02; LINC01571, 9.2E−01, |
| | 3.8E−02; POSTN, 9.6E−01, 3.8E−02; LOC105377699, 1.0E+00, 3.8E−02; LOC101930114, 1.0E+00, 8.3E−03; NFAM1, 1.1E+00, 2.6E−02; LOC340184, |
| | 1.2E+00, 1.6E−04; LOC729739, 1.3E+00, 1.1E−04; LOC101928447, 1.3E+00, 5.4E−03; ZMYND10, 1.3E+00, 7.7E−04; GAS2L2, 1.3E+00, 2.2E−03; LRRC70, |
| | 1.3E+00, 2.2E−02; LOC100652768, 1.4E+00, 2.0E−03; LOC105373893, 1.5E+00, 3.8E−02; LOC105369383, 1.5E+00, 3.9E−02; AP4B1−AS1, 1.5E+00, 8.3E−03; |
| | CCNA1, 1.5E+00, 7.3E−04; CFAP73, 1.5E+00, 3.4E−02; LOC101927087, 1.6E+00, 2.6E−02; SNORD54, 1.6E+00, 4.2E−03; HEMK1, 1.6E+00, 8.7E−03; |
| | MRS2P2, 1.6E+00, 3.5E−08; SKOR2, 1.7E+00, 1.6E−04; LOC107986181, 1.7E+00, 1.2E−03; CCL26, 1.7E+00, 4.5E−02; LINC00566, 1.7E+00, 5.7E−11; |
| | LOC101929657, 1.7E+00, 5.7E−11; LOC105369543, 1.7E+00, 5.7E−11; LOC105378179, 1.7E+00, 5.7E−11; LOC105378459, 1.7E+00, 5.7E−11; |
| | LOC105378927, 1.7E+00, 5.7E−11; TCL6, 1.7E+00, 5.7E−11; LOC105371056, 1.7E+00, 1.7E−08; LOC105374220, 1.7E+00, 3.7E−07; LOC101928627, |
| | 1.8E+00, 3.2E−04; LOC105374492, 1.8E+00, 8.3E−03; LOC107986103, 1.8E+00, 4.5E−05; ISY1−RAB43, 1.8E+00, 1.5E−02; LOC105379098, 1.8E+00, |
| | 1.0E−02; LOC105375537, 1.8E+00, 1.9E−03; LOC105375951, 1.8E+00, 2.7E−02; LOC105377180, 1.8E+00, 4.0E−02; RIBC1, 1.8E+00, 1.2E−02; AGR3, |
| | 1.9E+00, 1.9E−02; C1orf127, 1.9E+00, 7.3E−10; LOC105377698, 1.9E+00, 6.6E−08; LOC107985404, 1.9E+00, 1.7E−08; LINC00488, 2.0E+00, 1.8E−06; |
| | TPTE2P2, 2.0E+00, 4.3E−04; LOC105379086, 2.0E+00, 1.2E−07; SLC45A1, 2.0E+00, 5.8E−04; ADAM21, 2.0E+00, 2.7E−03; LOC105370803, 2.1E+00, |
| | 2.3E−02; VAV1, 2.1E+00, 3.8E−06; BORCS8, 2.2E+00, 4.5E−02; PSKH2, 2.2E+00, 7.1E−07; LOC101927151, 2.3E+00, 3.9E−03; LOC105377697, 2.3E+00, |
| | 7.8E−04; BCO2, 2.3E+00, 1.9E−02; FLJ13224, 2.3E+00, 6.0E−03; AKAP14, 2.3E+00, 1.5E−03; LOC105370259, 2.3E+00, 2.1E−02; C11orf88, 2.4E+00, 3.0E−06; |
| | SYNPO, 2.4E+00, 3.8E−02; LOC102724744, 2.4E+00, 5.0E−03; TTLL10-AS1, 2.4E+00, 5.9E−03; LOC101929895, 2.5E+00, 7.1E−06; LOC101929043, |
| | 2.5E+00, 4.5E−05; LOC105374957, 2.6E+00, 1.6E−04; IL5RA, 2.7E+00, 8.9E−07; LACTB, 2.7E+00, 4.5E−02; LOC101929006, 2.7E+00, 5.6E−03; |
| | LOC107987061, 2.8E+00, 4.2E−04; ACOX2, 2.8E+00, 3.4E−03; ZNF792, 2.9E+00, 3.4E−02; CFAP206, 2.9E+00, 8.8E−03; NR4A2, 2.9E+00, 4.1E−07; NLRX1, |
| | 2.9E+00, 6.4E−03; LOC105377227, 2.9E+00, 5.3E−04; SPATA18, 3.0E+00, 4.0E−02; PNMA2, 3.1E+00, 4.6E−02; DLEC1, 3.2E+00, 3.4E−04; ZNF428, |
| | 3.2E+00, 4.5E−02; KCNE1, 3.2E+00, 4.7E−05; C2orf81, 3.2E+00, 4.0E−02; ENKUR, 3.2E+00, 7.4E−03; LOC107986295, 3.3E+00, 4.7E−03; CYB5RL, |
| | 3.3E+00, 4.0E−02; MYO7A, 3.5E+00, 4.1E−06; CFAP52, 3.6E+00, 1.1E−02; CLIC6, 3.6E+00, 9.8E−03; LOC107986309, 3.6E+00, 2.5E−06; LOC105369838, |
| | 3.6E+00, 5.2E−07; DAW1, 3.6E+00, 3.5E−05; ADRA1A, 3.6E+00, 8.7E−03; ARHGAP40, 3.7E+00, 2.0E−09; FER1L5, 3.7E+00, 1.9E−02; TTC23L, 3.8E+00, |
| | 3.8E−02; CFAP61, 3.8E+00, 4.5E−02; CSGALNACT1, 3.8E+00, 3.8E−02; CFAP126, 3.9E+00, 1.3E−02; WBP2NL, 3.9E+00, 3.0E−02; TBXT, 4.0E+00, 4.6E−09; |
| | SERPINI2, 4.2E+00, 4.0E−03; LOC105377975, 4.2E+00, 3.7E−05; ADPRS, 4.2E+00, 3.4E−04; TEKT1, 4.2E+00, 5.5E−07; EFHC2, 4.2E+00, 3.8E−02; SPRTN, |
| | 4.3E+00, 4.9E−02; ADGB, 4.3E+00, 4.5E−06; CFAP100, 4.4E+00, 4.6E−04; SYNM, 4.4E+00, 2.5E−03; DPY19L2P1, 4.4E+00, 2.0E−03; RGS22, 4.4E+00, |
| | 1.8E−06; FAHD2A, 4.5E+00, 2.9E−02; VWA5B1, 4.6E+00, 3.9E−05; YPEL4, 4.6E+00, 3.2E−02; DNAH12, 4.6E+00, 1.5E−04; RSPH1, 4.6E+00, 3.8E−05; |
| | LOC105375138, 4.6E+00, 5.0E−08; ABCG2, 4.8E+00, 7.5E−03; EFCAB1, 4.8E+00, 8.9E−07; NOL4, 4.8E+00, 4.8E−02; TMEM231, 4.9E+00, 1.1E−02; |
| | CFAP45, 4.9E+00, 2.8E−05; ADAM12, 4.9E+00, 4.0E−02; TNFRSF11B, 5.0E+00, 1.9E−03; ARNTL, 5.0E+00, 4.0E−02; TOM1L2, 5.1E+00, 1.9E−02; DNAI1, |
| | 5.2E+00, 2.2E−06; LINC00869, 5.3E+00, 6.0E−04; AGBL1, 5.3E+00, 3.4E−07; FRMPD2, 5.3E+00, 1.6E−05; WDR66, 5.4E+00, 2.6E−02; CD109, 5.4E+00, |
| | 3.6E−02; CASC17, 5.5E+00, 2.6E−02; LOC107983974, 5.5E+00, 4.1E−02; TTC12, 5.5E+00, 2.2E−02; AKNA, 5.6E+00, 8.8E−03; CCDC146, 5.8E+00, 4.3E−02; |
| | MYH14, 5.9E+00, 1.7E−02; FYB2, 6.0E+00, 3.8E−02; ARMC4, 6.1E+00, 9.3E−03; CYP11A1, 6.1E+00, 3.0E−13; TTC29, 6.2E+00, 3.3E−03; CCDC30, |
| | 6.2E+00, 4.5E−02; TPPP3, 6.2E+00, 1.8E−09; LINC00632, 6.4E+00, 4.8E−05; DNAH9, 6.5E+00, 5.6E−03; C4orf47, 6.5E+00, 6.2E−04; CEP126, 6.5E+00, |
| | 9.9E−03; FANK1, 6.6E+00, 3.1E−04; DTHD1, 6.6E+00, 1.7E−12; SCD5, 6.7E+00, 1.3E−02; SPAG6, 6.7E+00, 3.5E−05; LRRC6, 6.7E+00, 3.9E−03; SPEF2, |
| | 6.8E+00, 4.3E−07; WDR78, 6.8E+00, 4.5E−02; SPOCK1, 6.9E+00, 3.8E−02; NEK10, 7.0E+00, 5.8E−03; PDE11A, 7.2E+00, 4.1E−03; LMNTD1, 7.3E+00, 6.6E−17; |
| | WDR49, 7.3E+00, 3.8E−03; CLSTN2, 7.3E+00, 4.2E−03; ARMC2, 7.4E+00, 1.9E−02; MAATS1, 7.5E+00, 5.8E−03; NKD1, 7.6E+00, 3.8E−02; CFAP46, |
| | 7.6E+00, 6.6E−06; CDHR3, 7.6E+00, 8.5E−03; CFAP43, 7.8E+00, 7.8E−05; CCDC173, 8.0E+00, 3.4E−04; LRRC9, 8.1E+00, 8.9E−07; LRGUK, 8.3E+00, |
| | 1.2E−02; DTWD2, 8.4E+00, 4.3E−03; SPAG17, 8.4E+00, 3.8E−06; TCTEX1D1, 8.5E+00, 1.6E−12; CLMN, 8.5E+00, 6.2E−05; DCAF10, 8.5E+00, 2.3E−02; |
| | LOC101928114, 8.6E+00, 1.9E−03; EFHC1, 8.7E+00, 3.8E−02; NCALD, 8.7E+00, 2.9E−02; SATB1, 8.7E+00, 4.0E−02; FRY, 8.7E+00, 1.7E−02; MACC1, |
| | 8.8E+00, 1.3E−02; KIF16B, 8.8E+00, 4.9E−02; VWA3A, 8.9E+00, 4.2E−12; CFAP47, 9.0E+00, 1.2E−09; C6orf118, 9.0E+00, 5.8E−17; WWTR1, 9.1E+00, |
| | 4.0E−03; ADARB2, 9.2E+00, 1.5E−02; RFX2, 9.2E+00, 8.9E−07; SPATA13, 9.2E+00, 3.8E−02; MDGA2, 9.3E+00, 2.6E−02; LRRIQ1, 9.5E+00, 3.3E−03; |
| | SAMD3, 9.5E+00, 3.7E−07; NEK11, 9.8E+00, 5.2E−04; NPTN, 9.9E+00, 2.7E−02; DNAH6, 9.9E+00, 3.7E−06; CFAP299, 1.0E+01, 3.4E−05; PIBF1, 1.0E+01, |
| | 4.8E−02; BTBD11, 1.0E+01, 1.4E−02; ARMC3, 1.0E+01, 3.7E−07; MBNL1, 1.0E+01, 3.8E−02; ZEB2, 1.1E+01, 4.5E−02; ATXN1, 1.1E+01, 4.5E−02; SEC31A, |
| | 1.1E+01, 4.0E−02; PKD1L1, 1.1E+01, 1.7E−06; EPB41L5, 1.1E+01, 4.5E−02; ATXN7, 1.1E+01, 1.5E−03; SPATA17, 1.1E+01, 2.4E−07; TRERF1, 1.1E+01, |
| | 3.8E−02; TTC6, 1.1E+01, 3.0E−02; CCDC141, 1.1E+01, 3.5E−08; MICU2, 1.1E+01, 3.0E−03; CDH10, 1.1E+01, 5.4E−04; FAM13A, 1.1E+01, 4.0E−02; NKAIN2, |
| | 1.1E+01, 4.6E−04; LMX1A, 1.2E+01, 3.0E−02; MOK, 1.2E+01, 4.1E−07; TNIK, 1.2E+01, 2.6E−02; SETBP1, 1.2E+01, 4.5E−02; COG5, 1.2E+01, 2.6E−02; |
| | MECOM, 1.2E+01, 2.9E−02; GALNT7, 1.2E+01, 4.9E−02; TMEM232, 1.2E+01, 9.1E−05; NSMCE2, 1.2E+01, 3.5E−02; RANBP17, 1.2E+01, 4.5E−02; |
| | ADAMTS6, 1.2E+01, 5.5E−03; ASAP1, 1.2E+01, 4.5E−02; NEDD4L, 1.2E+01, 3.6E−02; PRICKLE1, 1.2E+01, 1.9E−02; CFAP44, 1.3E+01, 2.8E−07; AGBL4, |
| | 1.3E+01, 2.6E−05; AGPAT4, 1.3E+01, 5.7E−03; VTI1A, 1.3E+01, 3.2E−02; PTK2, 1.3E+01, 1.7E−02; PTPN14, 1.3E+01, 3.5E−02; SHANK2, 1.3E+01, 4.1E−02; |
| | C11orf49, 1.3E+01, 5.7E−03; TLN2, 1.3E+01, 1.1E−02; LRBA, 1.4E+01, 3.8E−02; SDK1, 1.4E+01, 2.3E−02; GSK3B, 1.4E+01, 1.1E−02; HYDIN, 1.4E+01, |
| | 7.9E−10; AUTS2, 1.4E+01, 9.2E−04; PTPN13, 1.4E+01, 3.6E−02; FHIT, 1.4E+01, 1.0E−02; AKAP13, 1.4E+01, 1.1E−02; STK33, 1.4E+01, 6.5E−03; PAN3, |
| | 1.4E+01, 7.8E−03; CMTM8, 1.4E+01, 2.7E−03; PCDH9, 1.5E+01, 1.8E−03; SLIT3, 1.5E+01, 1.3E−03; PTPRD, 1.5E+01, 1.2E−02; MAGI1, 1.5E+01, 1.9E−03; |
| | MEF2A, 1.5E+01, 1.3E−03; TTC28, 1.5E+01, 8.7E−03; ADGRL3, 1.5E+01, 1.1E−03; DNAH5, 1.5E+01, 2.2E−11; TTN, 1.5E+01, 2.3E−03; NEAT1, 1.6E+01, |
| | 2.8E−03; ITGB8, 1.6E+01, 8.7E−07; EGFEM1P, 1.6E+01, 5.0E−03; ZC3H12B, 1.6E+01, 6.2E−03; TRIO, 1.6E+01, 3.2E−03; FBN2, 1.6E+01, 5.1E−03; LSAMP, |
| | 1.6E+01, 7.7E−04; RFX3, 1.7E+01, 1.3E−03; BTBD9, 1.7E+01, 2.6E−05; FNDC3B, 1.7E+01, 1.5E−03; SPAG16, 1.7E+01, 2.2E−05; PACRG, 1.7E+01, 6.6E−17; |
| | DNAH7, 1.8E+01, 4.0E−12; CADM1, 1.9E+01, 3.7E−04; NRXN3, 1.9E+01, 1.4E−04; PPP3CA, 2.0E+01, 2.6E−05; PRICKLE2, 2.0E+01, 3.5E−05; CFAP54, |
| | 2.1E+01, 7.8E−19; SLIT2, 2.9E+01, 6.6E−08; MAPK10, 3.0E+01, 2.2E−11; PLCL1, 3.3E+01, 8.4E−10 |
| 14 | PTMA, −8.5E+00, 1.1E−28; PFN1, −8.0E+00, 9.2E−10; RPL8, −7.3E+00, 3.0E−08; RPL13A, −7.2E+00, 3.2E−13; RPL13, −7.1E+00, 3.0E−09; CALM1, −6.9E+00, |
| | 2.2E−07; FTH1, −6.9E+00, 1.1E−08; HSP90AB1, −6.8E+00, 4.5E−11; ACTG1, −6.7E+00, 5.8E−08; RPL6, −6.5E+00, 1.8E−07; RPL15, −6.3E+00, 2.8E−06; |
| | RPLP1, −6.2E+00, 5.2E−07; HSP90AA1, −6.2E+00, 8.5E−12; TMSB4X, −6.2E+00, 9.9E−06; NPM1, −6.1E+00, 4.8E−10; RPL10, −6.0E+00, 2.5E−05; HMGA1, |
| | −5.9E+00, 2.3E−05; RPS18, −5.9E+00, 3.9E−06; RPS6, −5.9E+00, 7.7E−08; RACK1, −5.8E+00, 1.7E−05; RPS23, −5.7E+00, 6.5E−05; ACTB, −5.6E+00, 1.6E−08; |
| | RPS12, −5.6E+00, 1.1E−05; EEF2, −5.5E+00, 6.0E−05; KRT18, −5.5E+00, 2.1E−05; GAPDH, −5.5E+00, 3.2E−06; SERBP1, −5.4E+00, 5.5E−05; TPT1, −5.4E+00, |
| | 9.6E−05; H2AZ1, −5.4E+00, 2.3E−04; RPS15, −5.4E+00, 3.4E−04; GJA1, −5.4E+00, 3.0E−04; YBX1, −5.4E+00, 3.5E−04; RPL14, −5.3E+00, 1.8E−04; TMSB10, |
| | −5.2E+00, 3.0E−04; EEF1A1, −5.2E+00, 9.1E−12; RPL23, −5.2E+00, 1.6E−04; VCAN, −5.2E+00, 1.1E−04; RPL26, −5.2E+00, 6.9E−04; RPL21, −5.2E+00, 3.5E−04; |
| | SSB, −5.2E+00, 4.0E−04; RPS24, −5.1E+00, 3.1E−04; IDH1, −5.1E+00, 6.9E−04; STMN1, −5.1E+00, 9.6E−04; ATP5F1B, −5.1E+00, 8.8E−04; PRDX1, |
| | −5.1E+00, 2.0E−03; RPL9, −5.1E+00, 8.8E−04; RPS14, −5.0E+00, 1.3E−04; RPS8, −5.0E+00, 1.5E−05; RPL12, −5.0E+00, 1.5E−03; NUCKS1, −4.9E+00, 3.1E−04; |
| | TUBB, −4.9E+00, 1.1E−03; SKIL, −4.9E+00, 1.3E−03; RPL19, −4.9E+00, 5.8E−04; RPS7, −4.9E+00, 1.7E−03; HSP90B1, −4.9E+00, 2.9E−04; NCL, −4.8E+00, |
| | 6.6E−06; RPLPO, −4.8E+00, 1.9E−03; RPL10A, −4.7E+00, 1.7E−03; EEF1B2, −4.7E+00, 1.6E−03; EIF4G2, −4.7E+00, 9.9E−04; SERPINB9, −4.7E+00, 2.5E−03; |
| | GSTP1, −4.6E+00, 4.3E−03; HNRNPA1, −4.6E+00, 1.3E−04; RPL4, −4.6E+00, 1.5E−04; CCT5, −4.6E+00, 2.7E−03; RAN, −4.6E+00, 4.3E−03; RPL3, −4.6E+00, |
| | 9.9E−04; RPL37, −4.6E+00, 2.6E−03; RPL5, −4.6E+00, 1.7E−03; RPL35A, −4.6E+00, 3.9E−03; TUBA1B, −4.6E+00, 2.3E−03; MYL6, −4.6E+00, 4.4E−03; BTF3, |
| | −4.5E+00, 2.1E−03; H3F3B, −4.5E+00, 4.1E−03; HSPA8, −4.5E+00, 2.4E−03; RPL18, −4.5E+00, 6.3E−03; RPL32, −4.5E+00, 6.2E−03; PODXL, −4.4E+00, 5.2E−03; |
| | CD24, −4.4E+00, 2.0E−03; SPP1, −4.4E+00, 9.8E−04; XRCC6, −4.4E+00, 6.4E−03; RPS3, −4.4E+00, 7.0E−03; L1TD1, −4.4E+00, 6.0E−05; RPL7A, −4.4E+00, |
| | 9.4E−03; ILF3, −4.4E+00, 8.4E−03; RPLP2, −4.3E+00, 7.2E−03; RPS16, −4.3E+00, 9.3E−03; NACA, −4.3E+00, 8.5E−03; RPS5, −4.3E+00, 9.5E−03; DNMT3B, |
| | −4.3E+00, 2.6E−04; KRT8, −4.3E+00, 3.7E−03; PMAIP1, −4.3E+00, 4.1E−04; SUPT16H, −4.3E+00, 1.0E−02; SERPINH1, −4.3E+00, 1.9E−03; SET, −4.3E+00, |
| | 1.0E−03; SUMO2, −4.3E+00, 4.7E−03; EID1, −4.3E+00, 1.3E−03; UBA52, −4.3E+00, 7.2E−03; CYP51A1, −4.3E+00, 7.5E−03; UGP2, −4.2E+00, 1.2E−03; HSPA5, |
| | −4.2E+00, 3.3E−02; PSMA7, −4.2E+00, 5.9E−03; PPIA, −4.2E+00, 1.6E−02; RPL34, −4.2E+00, 1.3E−02; HNRNPA2B1, −4.2E+00, 6.4E−05; RPL35, −4.1E+00, |
| | 2.1E−02; ALDOA, −4.1E+00, 4.2E−03; ZFP42, −4.1E+00, 1.6E−02; CFL1, −4.1E+00, 9.4E−03; TPM3, −4.1E+00, 1.7E−02; PARP1, −4.1E+00, 3.6E−02; TDGF1, |
| | −4.1E+00, 1.6E−02; YWHAB, −4.1E+00, 1.0E−02; NASP, −4.0E+00, 1.0E−02; RPS15A, −4.0E+00, 2.0E−02; HSPD1, −4.0E+00, 7.9E−03; PLS3, −4.0E+00, 2.3E−02; |
| | LOC105377901, −4.0E+00, 6.9E−03; ATP5F1A, −4.0E+00, 2.2E−02; TALDO1, −4.0E+00, 3.4E−04; RPS28, −3.9E+00, 2.0E−02; FLNA, −3.9E+00, 2.4E−02; |
| | LIN28A, −3.9E+00, 9.3E−04; DPYSL3, −3.9E+00, 3.0E−02; FTL, −3.9E+00, 3.1E−02; RPS21, −3.9E+00, 1.7E−02; RPL18A, −3.8E+00, 3.4E−02; RPL24, |
| | −3.8E+00, 4.1E−02; PEBP1, −3.8E+00, 1.3E−02; SRP14, −3.8E+00, 1.6E−02; PKM, −3.8E+00, 4.1E−02; DPPA4, −3.8E+00, 3.6E−02; DHCR24, −3.7E+00, 4.8E−02; |
| | RPS27A, −3.7E+00, 4.3E−02; TPI1, −3.7E+00, 4.0E−02; GARS1, −3.7E+00, 3.2E−02; UBE2I, −3.7E+00, 7.0E−03; MARCKSL1, −3.7E+00, 4.8E−03; TMED2, |
| | −3.7E+00, 7.2E−03; SLC25A5, −3.7E+00, 3.6E−03; ACVR2B, −3.6E+00, 3.3E−02; CNN3, −3.6E+00, 2.9E−02; SSRP1, −3.6E+00, 1.5E−02; PDLIM1, −3.6E+00, |
| | 2.7E−03; GL01, −3.5E+00, 3.6E−02; PSMA4, −3.5E+00, 3.2E−02; PABPC1, −3.5E+00, 5.9E−03; YWHAZ, −3.5E+00, 5.0E−02; RPS26, −3.5E+00, 4.3E−02; |
| | SF3A3, −3.4E+00, 1.9E−02; TPM2, −3.4E+00, 4.6E−03; IDI1, −3.3E+00, 4.7E−02; KRT19, −3.3E+00, 4.3E−03; C14orf166, −3.3E+00, 4.9E−02; FSCN1, |
| | −3.2E+00, 3.0E−02; IFITM1, −3.2E+00, 3.3E−02; PRDX3, −3.2E+00, 2.6E−02; BAG6, −3.1E+00, 8.5E−04; SCG3, −3.1E+00, 2.5E−03; H2BC9, −3.0E+00, 3.6E−02; |
| | TAF7, −3.0E+00, 5.0E−02; RDH11, −2.9E+00, 2.4E−02; LSM4, −2.9E+00, 2.4E−02; COX3, −2.8E+00, 1.2E−04; LIN7C, −2.8E+00, 3.2E−02; HS6ST3, −2.8E+00, |
| | 2.0E−02; RPL41, −2.8E+00, 4.3E−02; EPPK1, −2.7E+00, 1.7E−02; TUBB3, −2.6E+00, 3.3E−02; PTK7, −2.6E+00, 4.6E−02; GAL, −2.6E+00, 2.1E−03; PES1, |
| | −2.3E+00, 5.0E−03; MRPL44, −2.2E+00, 2.4E−02; DIPK2A, −2.1E+00, 2.4E−02; COX1, −2.0E+00, 4.0E−03; OLFML3, −1.9E+00, 3.7E−02; NANOG, −1.9E+00, |
| | 2.5E−02; ND4, −1.5E+00, 3.3E−02; FKRP, −1.4E+00, 1.6E−02; LOC107986048, 3.6E−01, 3.6E−02; LOC101928126, 4.0E−01, 4.8E−02; LOC105372112, 4.8E−01, |
| | 4.3E−02; LOC107984625, 5.0E−01, 4.5E−02; SNORD45A, 5.1E−01, 2.6E−02; IL17B, 5.9E−01, 2.6E−02; LOC107986088, 6.2E−01, 4.6E−03; NCR3LG1, |
| | 6.4E−01, 3.6E−02; LOC105378646, 6.4E−01, 3.7E−02; ALDH5A1, 7.2E−01, 3.7E−02; ADCY8, 7.8E−01, 3.8E−02; PPP1R16A, 9.9E−01, 3.7E−03; CASP10, |
| | 1.1E+00, 3.8E−02; KCNMB2-AS1, 1.1E+00, 4.3E−02; LOC101927770, 1.2E+00, 1.2E−02; CHRM2, 1.2E+00, 2.0E−02; SOAT2, 1.2E+00, 1.6E−02; |
| | LOC105373835, 1.2E+00, 3.7E−02; CCDC60, 1.2E+00, 2.9E−03; TLR8-AS1, 1.2E+00, 3.1E−02; CSF3R, 1.3E+00, 1.3E−02; KHDC1L, 1.4E+00, 2.4E−02; |
| | LOC105370826, 1.4E+00, 2.7E−03; LOC105373187, 1.4E+00, 4.8E−02; CCDC190, 1.4E+00, 1.9E−02; C9orf41-AS1, 1.5E+00, 1.9E−02; LOC344887, |
| | 1.5E+00, 1.0E−03; LOC105372915, 1.6E+00, 1.4E−02; LOC105378005, 1.6E+00, 4.5E−02; LOC102724168, 1.6E+00, 3.5E−03; LOC105376923, 1.7E+00, |
| | 2.7E−03; PLCG2, 1.7E+00, 1.4E−02; PRLR, 1.8E+00, 1.3E−02; LINC00383, 1.8E+00, 1.0E−03; CNNM4, 1.9E+00, 7.2E−03; LOC105378516, 1.9E+00, 3.6E−03; |
| | LOC105371143, 1.9E+00, 4.8E−02; GATA3, 1.9E+00, 1.3E−02; ST8SIA2, 1.9E+00, 4.6E−02; MFAP3L, 1.9E+00, 4.6E−02; LOC105376584, 2.0E+00, |
| | 1.3E−02; TMC7, 2.0E+00, 2.7E−02; PRSS23, 2.1E+00, 2.8E−02; LOC105371070, 2.1E+00, 3.1E−03; RNF138P1, 2.1E+00, 4.1E−02; HAVCR1, 2.1E+00, |
| | 1.3E−04; LOC107986062, 2.2E+00, 1.9E−03; LOC107987296, 2.2E+00, 1.2E−02; RRAGB, 2.2E+00, 1.6E−02; SLC25A43, 2.2E+00, 4.4E−02; LOC101928911, |
| | 2.2E+00, 4.5E−05; CTSL, 2.2E+00, 3.3E−02; GPC5, 2.2E+00, 4.8E−02; PPFIBP2, 2.2E+00, 4.6E−03; LOC107986706, 2.3E+00, 1.2E−03; LOC107987043, |
| | 2.3E+00, 1.7E−02; MRPS30-DT, 2.4E+00, 1.4E−03; FAM193B, 2.4E+00, 5.0E−02; LOC101927056, 2.4E+00, 1.9E−02; FOXK1, 2.5E+00, 3.4E−02; |
| | LOC105369427, 2.5E+00, 2.1E−05; DLGAP3, 2.5E+00, 2.1E−02; CA10, 2.5E+00, 5.2E−03; LOC105372950, 2.5E+00, 4.6E−04; NUDT4, 2.5E+00, 4.3E−02; |
| | TMEM168, 2.6E+00, 2.8E−03; GDPD1, 2.6E+00, 2.1E−02; CLEC2L, 2.6E+00, 4.0E−04; TNRC18, 2.6E+00, 2.2E−02; LOC105378657, 2.7E+00, 6.9E−03; |
| | CD48, 2.7E+00, 3.6E−03; RASGRF2, 2.7E+00, 4.0E−02; LOC107987100, 2.8E+00, 1.3E−02; TEC, 2.8E+00, 1.9E−02; TPTE2P1, 2.8E+00, 4.5E−03; LRP4, |
| | 2.8E+00, 1.7E−02; SLC35F2, 2.8E+00, 1.5E−02; UMAD1, 2.8E+00, 3.1E−02; LOC107986724, 2.8E+00, 8.1E−03; WDR11-AS1, 2.8E+00, 2.3E−04; SLC01C1, |
| | 2.8E+00, 5.0E−02; ANXA3, 2.8E+00, 1.3E−02; CPNE8, 2.9E+00, 1.2E−02; MINDY2, 2.9E+00, 4.3E−02; MIR4435-2HG, 3.0E+00, 2.1E−03; CDK14, 3.0E+00, |
| | 2.5E−02; C2CD2, 3.0E+00, 2.3E−03; DIP2A, 3.0E+00, 3.1E−02; TMEM62, 3.0E+00, 1.2E−04; KCNK1, 3.1E+00, 4.1E−03; TXK, 3.1E+00, 1.1E−05; CASP8, |
| | 3.1E+00, 4.0E−04; ZFHX3, 3.1E+00, 1.2E−02; CDH8, 3.1E+00, 2.5E−02; SLC49A4, 3.1E+00, 1.1E−03; TVP23C-CDRT4, 3.1E+00, 8.5E−03; ERV3−1, 3.1E+00, |
| | 3.4E−04; GS1-124K5.11, 3.1E+00, 1.4E−02; FEZ2, 3.1E+00, 1.6E−02; ATP6V1H, 3.1E+00, 1.2E−02; ERBB4, 3.1E+00, 2.1E−02; PPP1R12B, 3.2E+00, 3.2E−02; |
| | LINC00871, 3.2E+00, 6.0E−05; STXBP5, 3.2E+00, 2.4E−02; SLC39A11, 3.2E+00, 3.2E−02; ASXL3, 3.2E+00, 1.2E−04; ST3GAL1, 3.2E+00, 1.6E−03; |
| | ZNF516, 3.3E+00, 2.7E−03; LOC105370504, 3.3E+00, 7.6E−03; FA2H, 3.3E+00, 1.8E−03; DLG5, 3.3E+00, 4.5E−02; SENP5, 3.3E+00, 2.7E−02; SEMA6A, |
| | 3.4E+00, 4.0E−02; CLEC16A, 3.4E+00, 5.2E−03; LOC107984449, 3.4E+00, 3.0E−08; CNOT2, 3.4E+00, 4.6E−02; PDXDC2P, 3.4E+00, 2.5E−03; TMEM161B, |
| | 3.4E+00, 4.8E−03; BMP6, 3.4E+00, 2.2E−06; JMY, 3.5E+00, 3.4E−02; LOC101928283, 3.5E+00, 1.2E−03; KHDC1, 3.5E+00, 7.3E−03; DERA, 3.5E+00, 6.8E−03; |
| | SENP7, 3.5E+00, 2.8E−04; SNAPC3, 3.5E+00, 5.6E−03; TAF1, 3.5E+00, 9.7E−03; LPIN1, 3.5E+00, 7.2E−03; TCF12, 3.6E+00, 2.9E−02; TET3, 3.6E+00, |
| | 2.4E−02; ZSWIM6, 3.6E+00, 4.0E−02; GAREM1, 3.6E+00, 9.7E−04; PPP2R5C, 3.6E+00, 8.6E−03; TYW1, 3.6E+00, 6.6E−03; GPATCH2, 3.6E+00, 2.4E−02; |
| | MTHFD1L, 3.6E+00, 3.9E−02; RALGAPA1, 3.6E+00, 1.2E−02; REV3L, 3.6E+00, 2.2E−02; LOC107985037, 3.7E+00, 1.9E−02; DLEU1, 3.7E+00, 7.3E−03; |
| | CASK, 3.7E+00, 4.9E−02; ANAPC10, 3.7E+00, 1.8E−03; ERCC8, 3.7E+00, 3.7E−03; C1GALT1, 3.7E+00, 2.6E−02; MAP4K3, 3.7E+00, 2.8E−02; ATXN7, |
| | 3.7E+00, 2.2E−03; KIAA0232, 3.7E+00, 1.9E−02; TDRD3, 3.7E+00, 5.2E−03; RBM33, 3.7E+00, 7.2E−03; SNX24, 3.7E+00, 4.0E−03; PLSCR4, 3.7E+00, |
| | 1.6E−04; NPSR1-AS1, 3.8E+00, 3.5E−02; VAV2, 3.8E+00, 4.5E−02; PRIM2, 3.8E+00, 3.6E−02; TNKS, 3.8E+00, 2.8E−02; LARP1B, 3.8E+00, 2.5E−02; AGAP1, |
| | 3.8E+00, 4.6E−02; DUSP16, 3.8E+00, 2.7E−02; RAF1, 3.8E+00, 9.1E−03; AHNAK, 3.9E+00, 4.1E−02; KIAA1671, 3.9E+00, 1.4E−02; PCNX2, 3.9E+00, 3.6E−02; |
| | PAN3, 3.9E+00, 4.1E−02; ATP8B1, 3.9E+00, 6.8E−05; ZNF138, 3.9E+00, 1.1E−02; TRAPPC9, 3.9E+00, 3.8E−02; FTX, 3.9E+00, 6.6E−03; PSPC1, |
| | 3.9E+00, 3.8E−02; SGCD, 4.0E+00, 2.4E−02; BICD1, 4.0E+00, 3.1E−04; ZFAND3, 4.0E+00, 4.0E−02; CRACD, 4.0E+00, 3.5E−03; ATP2B1, 4.0E+00, 9.8E−03; |
| | SCFD2, 4.0E+00, 3.7E−03; SLC12A3, 4.0E+00, 1.4E−08; FRAS1, 4.0E+00, 6.3E−03; POLA1, 4.0E+00, 2.9E−02; PHKB, 4.0E+00, 1.1E−02; UBE2E1, 4.0E+00, |
| | 1.9E−02; GRB10, 4.0E+00, 7.5E−03; OPHN1, 4.0E+00, 1.5E−03; WDFY3, 4.1E+00, 1.4E−02; CTNNA3, 4.1E+00, 1.7E−02; LRRC8D, 4.1E+00, 1.6E−02; |
| | PPARGC1B, 4.1E+00, 4.0E−03; ERBIN, 4.1E+00, 1.7E−02; FARP1, 4.1E+00, 2.7E−02; FAM160A1, 4.1E+00, 2.2E−02; KLHL18, 4.1E+00, 1.0E−03; SLC25A16, |
| | 4.1E+00, 2.4E−03; RREB1, 4.1E+00, 1.2E−03; MAN1A2, 4.1E+00, 2.9E−03; UTY, 4.2E+00, 1.4E−02; DANT2, 4.2E+00, 1.5E−02; N4BP1, 4.2E+00, 1.1E−03; |
| | ITPR2, 4.2E+00, 1.4E−02; LINC01057, 4.2E+00, 1.3E−03; SLC4A7, 4.2E+00, 4.9E−03; KCNK2, 4.2E+00, 6.5E−05; FBXL20, 4.3E+00, 1.9E−03; ROBO1, |
| | 4.3E+00, 2.1E−02; EIF4G3, 4.3E+00, 5.8E−03; FER, 4.3E+00, 8.1E−03; MED13L, 4.3E+00, 7.9E−03; ASAP1, 4.3E+00, 1.3E−02; ASH1L, 4.3E+00, 1.1E−02; |
| | NCOA3, 4.3E+00, 1.3E−03; PATJ, 4.4E+00, 5.8E−03; HSD17B12, 4.4E+00, 1.3E−02; DNAJB4, 4.4E+00, 3.7E−03; DEPTOR, 4.4E+00, 7.1E−04; ZDHHC17, |
| | 4.4E+00, 1.4E−03; CRPPA, 4.4E+00, 2.9E−06; MALAT1, 4.4E+00, 3.2E−13; TNRC6C, 4.4E+00, 4.0E−03; VWA8, 4.4E+00, 3.3E−03; TSC22D1, 4.4E+00, 7.2E−03; |
| | FMN1, 4.5E+00, 2.0E−07; ST3GAL6, 4.5E+00, 7.1E−04; COP1, 4.5E+00, 4.3E−03; WDR11, 4.5E+00, 1.3E−03; DDHD1, 4.5E+00, 3.4E−04; NHS, 4.5E+00, |
| | 1.1E−02; PYGB, 4.5E+00, 5.3E−04; MYO9A, 4.5E+00, 4.1E−03; SPRED2, 4.5E+00, 2.6E−04; LOC101927768, 4.6E+00, 4.1E−05; MOB3B, 4.6E+00, 6.5E−05; |
| | TMTC1, 4.6E+00, 4.9E−03; NOVA1-AS1, 4.6E+00, 2.2E−07; IL12A-AS1, 4.6E+00, 9.2E−07; SCAPER, 4.6E+00, 2.8E−03; SPIDR, 4.7E+00, 4.1E−03; MEI4, |
| | 4.7E+00, 2.3E−04; FLVCR2, 4.7E+00, 2.1E−07; TBC1D22A, 4.7E+00, 2.0E−03; BARD1, 4.7E+00, 2.3E−03; RIN2, 4.7E+00, 7.2E−06; LOC105375815, |
| | 4.7E+00, 4.9E−06; SULF1, 4.8E+00, 7.2E−03; LRBA, 4.8E+00, 4.5E−04; DIAPH3, 4.8E+00, 4.3E−03; NTRK3, 4.8E+00, 1.6E−06; SDHAP3, 4.8E+00, 6.9E−04; |
| | RNF115, 4.8E+00, 1.3E−04; ZFYVE9, 4.8E+00, 7.1E−04; RBMS1, 4.8E+00, 2.5E−03; KDM4C, 4.8E+00, 3.0E−03; UBAC2, 5.0E+00, 2.5E−03; RCBTB1, |
| | 5.0E+00, 3.1E−06; CHCHD3, 5.0E+00, 1.7E−03; SHB, 5.0E+00, 2.3E−05; MAP4, 5.1E+00, 5.4E−04; LOC107986215, 5.1E+00, 1.7E−06; BTRC, 5.1E+00, |
| | 1.7E−04; GPD2, 5.2E+00, 2.3E−04; GAS7, 5.3E+00, 4.4E−07; CNTN6, 5.3E+00, 3.7E−09; FGF13, 5.3E+00, 6.3E−03; SCN2A, 5.3E+00, 2.1E−12; CDC42BPA, |
| | 5.4E+00, 3.3E−04; LOC728755, 5.5E+00, 1.0E−03; LOC101927967, 5.5E+00, 3.1E−07; MAN1C1, 5.5E+00, 5.7E−07; ARFGEF1, 5.6E+00, 4.6E−05; LSAMP, |
| | 5.6E+00, 2.9E−04; TENM4, 5.6E+00, 1.8E−04; RNGTT, 5.6E+00, 2.2E−04; GPHN, 5.6E+00, 2.1E−04; PDZD2, 5.7E+00, 4.5E−05; CHD2, 5.7E+00, 8.5E−05; |
| | ME1, 5.7E+00, 7.7E−08; KIAA1217, 5.8E+00, 2.6E−04; LOC105373153, 5.9E+00, 4.6E−07; PCAT14, 6.0E+00, 2.4E−04; SHROOM3, 6.2E+00, 5.1E−05; |
| | LOC105375199, 6.3E+00, 2.2E−14; VPS13B, 6.3E+00, 3.2E−06; UBR1, 6.4E+00, 6.2E−07; GRIK3, 6.4E+00, 2.8E−06; PITPNC1, 6.4E+00, 1.3E−05; |
| | LOC339862, 6.5E+00, 1.1E−05; CACNA1C, 6.8E+00, 1.7E−09; PCMTD1, 6.9E+00, 1.9E−07; ELM01, 7.0E+00, 3.6E−07; MAST2, 7.0E+00, 5.8E−08; KMT2C, |
| | 7.2E+00, 2.1E−07; ATP9B, 7.2E+00, 3.1E−08; ARL15, 7.2E+00, 5.1E−07; AIRE, 7.2E+00, 2.3E−16; ADAMTSL1, 7.6E+00, 1.6E−12; AGL, 7.7E+00, 1.7E−07; |
| | YPEL2, 7.7E+00, 5.8E−09; LOC105374013, 7.8E+00, 1.3E−09; NEAT1, 8.0E+00, 1.6E−06; RIMKLB, 8.2E+00, 4.0E−09; RPTOR, 8.4E+00, 8.5E−12; |
| | LINC01250, 8.6E+00, 8.1E−12; SCN3A, 8.6E+00, 1.6E−23; TULP4, 8.7E+00, 2.7E−11; DNAJC15, 8.7E+00, 4.4E−11; RBFOX1, 9.3E+00, 5.2E−13; ST6GAL1, |
| | 9.5E+00, 1.1E−19; ATG10, 1.0E+01, 6.6E−13; LOC105375334, 1.0E+01, 8.9E−14; PARK2, 1.1E+01, 1.6E−11; CHODL, 1.1E+01, 7.3E−20; FAF1, 1.1E+01, |
| | 9.4E−17; ANKDD1B, 1.2E+01, 3.0E−25; CACNA2D3, 1.2E+01, 2.0E−21; LOC105376755, 1.5E+01, 6.1E−23 |
| 15 | H2AC11, −1.6E+01, 3.7E−04; H2AC14, −1.2E+01, 2.2E−02; H3C11, −1.0E+01, 1.7E−02; FRMPD1, 3.4E+00, 1.9E−02; VASH1, 4.5E+00, 4.9E−02; SHC3, |
| | 6.5E+00, 3.9E−02; EGFLAM, 6.7E+00, 7.5E−04; LHX8, 7.0E+00, 5.5E−03; GRIK3, 7.8E+00, 1.3E−02; ZFHX4, 8.6E+00, 3.8E−02; LOC105374322, 8.9E+00, |
| | 1.5E−02; NTNG1, 8.9E+00, 1.3E−02; PLCL1, 9.0E+00, 5.5E−03; ZBTB20, 9.6E+00, 2.4E−02; LOC105377703, 9.8E+00, 3.9E−04; DGKB, 9.9E+00, 4.2E−02; |
| | PCDH15, 1.0E+01, 2.4E−03; PITX2, 1.1E+01, 4.0E−03; ANTXR2, 1.1E+01, 6.1E−03; CNTN6, 1.1E+01, 4.0E−04; TNFRSF19, 1.1E+01, 5.3E−05; NCOA1, |
| | 1.1E+01, 3.8E−02; CNTN4, 1.2E+01, 4.0E−02; PTK2, 1.2E+01, 3.8E−02; SOX5, 1.2E+01, 4.4E−02; CDH2, 1.2E+01, 4.6E−02; MARCHF1 , 1.2E+01, 2.5E−03; |
| | MAP2, 1.2E+01, 2.6E−02; PPP2R2B, 1.3E+01, 1.8E−02; NRCAM, 1.3E+01, 6.1E−03; TLE4, 1.3E+01, 1.1E−02; CELF2, 1.3E+01, 6.2E−03; FBN2, 1.3E+01, |
| | 7.5E−03; NRG1, 1.4E+01, 1.3E−02; LTBP1, 1.4E+01, 9.2E−03; SEMA3A, 1.4E+01, 2.7E−03; PKNOX2, 1.4E+01, 2.1E−04; SEMA3D, 1.4E+01, 2.7E−05; |
| | HDAC9, 1.5E+01, 4.5E−04; PCDH7, 1.5E+01, 1.1E−03; CADPS, 1.5E+01, 6.6E−04; GLCCI1, 1.5E+01, 4.5E−05; DOK6, 1.5E+01, 8.8E−07; CHN2, 1.6E+01, |
| | 4.5E−05; CDK14, 1.6E+01, 2.1E−04; GPC6, 1.6E+01, 2.1E−04; ATP2B1, 1.7E+01, 4.5E−05; DACH1, 1.8E+01, 1.1E−04; NOVA1-AS1, 2.1E+01, 7.8E−09; |
| | SEMA6D, 2.2E+01, 1.6E−07 |
| 16 | ADAMTS18, −7.4E+00, 3.9E−02; ERBB4, 6.5E+01, 2.0E−03; LOC102724340, 4.4E+01, 3.5E−02; LOC107984125, 4.7E+01, 1.2E−02 |
| 17 | B9D1, −1.3E+00, 4.6E−02; ADO, −1.1E+00, 4.0E−02; BOLA3, −1.1E+00, 4.0E−02; FAM219A, −1.1E+00, 4.0E−02; GTF2A2, −1.1E+00, 4.0E−02; IFITM3, |
| | −1.1E+00, 4.0E−02; IKZF2, −1.1E+00, 4.0E−02; ITM2A, −1.1E+00, 4.0E−02; MGAT1, −1.1E+00, 4.0E−02; PFKL, −1.1E+00, 4.0E−02; PLPP5, −1.1E+00, 4.0E−02; |
| | PLPP6, −1.1E+00, 4.0E−02; PYCR2, −1.1E+00, 4.0E−02; RAB11FIP1, −1.1E+00, 4.0E−02; RBM23, −1.1E+00, 4.0E−02; ZNF605, −1.1E+00, 4.0E−02; NCKAP5, |
| | 5.2E+00, 3.4E−02 |

### Table 19A-19D.—Predicted TF Combinations for Reference Cell Types.

TABLE 19A
Hierarchical clustering of TF ORFs for TF Atlas
differentiated cells into 365 clusters.

| TF ORF | Cluster |
|---|---|
| TFORF0483-ZNF223 | 1 |
| TFORF1479-TP63 | 1 |
| TFORF2682-ZMYND11 | 1 |
| TFORF2467-IRF5 | 2 |
| TFORF2706-TFCP2L1 | 2 |
| TFORF0524-PAX4 | 3 |
| TFORF0974-GFI1B | 3 |
| TFORF1923-ZBTB20 | 3 |
| TFORF2132-LCOR | 3 |
| TFORF3344-ZNF280A | 3 |
| TFORF0501-EGR4 | 4 |
| TFORF0606-PUF60 | 4 |
| TFORF1157-DMBX1 | 4 |
| TFORF1412-ZNF562 | 4 |
| TFORF1592-ZNF771 | 4 |
| TFORF0292-MEF2B | 5 |
| TFORF0519-PAX5 | 5 |
| TFORF0738-ZNF155 | 5 |
| TFORF0853-ZIC5 | 5 |
| TFORF1030-RARG | 5 |
| TFORF1165-PRKCD | 5 |
| TFORF1273-DPF2 | 5 |
| TFORF1370-ZNF141 | 5 |
| TFORF2107-ZBTB37 | 5 |
| TFORF2328-CIZ1 | 5 |
| TFORF2333-ZNF491 | 5 |
| TFORF2411-KLF3 | 5 |
| TFORF2855-MAPK8IP1 | 5 |
| TFORF3109-ELF5 | 5 |
| TFORF3137-HEY1 | 5 |
| TFORF0054-ZNF679 | 6 |
| TFORF0843-ZSCAN10 | 6 |
| TFORF0849-ZIC4 | 6 |
| TFORF1362-SP100 | 6 |
| TFORF1535-ZBTB44 | 6 |
| TFORF0514-PAX5 | 7 |
| TFORF0515-PAX5 | 7 |
| TFORF0517-PAX5 | 7 |
| TFORF0518-PAX5 | 7 |
| TFORF0537-PAX2 | 7 |
| TFORF0538-PAX2 | 7 |
| TFORF0742-ZNF485 | 7 |
| TFORF1277-ZNF587 | 7 |
| TFORF1302-ZMIZ2 | 7 |
| TFORF1921-ZBTB22 | 7 |
| TFORF2225-ZNF343 | 7 |
| TFORF2501-PML | 7 |
| TFORF3201-ZNF829 | 7 |
| TFORF3418-NFE2L2 | 7 |
| TFORF3504-ZNF543 | 7 |
| TFORF0478-ZNF222 | 8 |
| TFORF0552-ZNF611 | 8 |
| TFORF1981-TBX4 | 8 |
| TFORF2882-XBP1 | 8 |
| TFORF0336-ZNF91 | 9 |
| TFORF0698-BATF2 | 9 |
| TFORF0837-ZSCAN18 | 9 |
| TFORF0901-ZNF382 | 9 |
| TFORF1325-KLF16 | 9 |
| TFORF1421-ZNF211 | 9 |
| TFORF2188-ZNF410 | 9 |
| TFORF2214-CREB3L4 | 9 |
| TFORF2584-MIXL1 | 9 |
| TFORF2942-ESRRG | 9 |
| TFORF0165-SIX5 | 10 |
| TFORF0397-ZNF2 | 10 |
| TFORF0507-CDK2 | 10 |
| TFORF1554-SP110 | 10 |
| TFORF2181-ZNF417 | 10 |
| TFORF3324-XRCC6 | 10 |
| TFORF3441-MNAT1 | 10 |
| TFORF0464-TRIM22 | 11 |
| TFORF0523-PAX5 | 11 |
| TFORF0610-RBPJ | 11 |
| TFORF1806-TBX15 | 11 |
| TFORF1849-ETS1 | 11 |
| TFORF1930-XRCC6 | 11 |
| TFORF2124-ZNF747 | 11 |
| TFORF2372-ZNF468 | 11 |
| TFORF2513-PDCD2 | 11 |
| TFORF2621-ZXDA | 11 |
| TFORF2970-RORC | 11 |
| TFORF3464-KLF7 | 11 |
| TFORF3500-BATF3 | 11 |
| TFORF0588-FOXO3 | 12 |
| TFORF1341-POU5F1 | 12 |
| TFORF1493-FOSL1 | 12 |
| TFORF1680-GTF2IRD2B | 12 |
| TFORF1936-MEIS2 | 12 |
| TFORF2340-MITF | 12 |
| TFORF0324-LCORL | 13 |
| TFORF1081-NOBOX | 13 |
| TFORF3012-HSF2 | 13 |
| TFORF3222-FOXS1 | 13 |
| TFORF3385-ZIC3 | 13 |
| TFORF0398-ZNF2 | 14 |
| TFORF1000-ZNF302 | 14 |
| TFORF1051-HKR1 | 14 |
| TFORF1987-ZNF534 | 14 |
| TFORF0632-VPS72 | 15 |
| TFORF0835-HSFY1 | 15 |
| TFORF3292-MXD3 | 15 |
| TFORF0046-MAX | 16 |
| TFORF1256-TP53 | 16 |
| TFORF1299-MAEL | 16 |
| TFORF2098-ZKSCAN1 | 16 |
| TFORF2482-MXD3 | 16 |
| TFORF3403-ZNF488 | 16 |
| TFORF1880-CREBZF | 17 |
| TFORF2731-ZNF669 | 17 |
| TFORF3483-PBX4 | 17 |
| TFORF0630-NR112 | 18 |
| TFORF2717-TFCP2 | 18 |
| TFORF3429-ZNF302 | 18 |
| TFORF3440-ING1 | 18 |
| TFORF0154-THAP7 | 19 |
| TFORF0547-PAX9 | 19 |
| TFORF0851-ZIC4 | 19 |
| TFORF2422-RBPJL | 19 |
| TFORF3187-RAD21 | 19 |
| TFORF0127-SP8 | 20 |
| TFORF0156-EWSR1 | 20 |
| TFORF1224-AKNA | 20 |
| TFORF2065-NKX2-5 | 20 |
| TFORF2611-ZNF277 | 20 |
| TFORF2629-INSM2 | 20 |
| TFORF2665-ZBED2 | 20 |
| TFORF2990-NFE2L1 | 20 |
| TFORF1573-ZNF684 | 21 |
| TFORF2201-POU4F2 | 21 |
| TFORF2756-BARHL2 | 21 |
| TFORF0204-ELK4 | 22 |
| TFORF0414-CTCFL | 22 |
| TFORF1677-RUNX1T1 | 22 |
| TFORF2966-NROB1 | 22 |
| TFORF0493-ZSCAN2 | 23 |
| TFORF1489-NR6A1 | 23 |
| TFORF2937-FOS | 23 |
| TFORF0532-PAX1 | 24 |
| TFORF0981-KLF6 | 24 |
| TFORF1967-PPARD | 24 |
| TFORF2879-ELF2 | 24 |
| TFORF0126-CXXC1 | 25 |
| TFORF1105-GTF2IRD2 | 25 |
| TFORF1621-ZNF512 | 25 |
| TFORF1741-ZNF768 | 25 |
| TFORF2439-YEATS4 | 25 |
| TFORF3347-PITX1 | 25 |
| TFORF0543-ZFAT | 26 |
| TFORF1541-ZBTB49 | 26 |
| TFORF0678-PRDM14 | 27 |
| TFORF1289-GMEB2 | 27 |
| TFORF1727-ZNF692 | 27 |
| TFORF1335-POU3F2 | 28 |
| TFORF2821-IKZF5 | 28 |
| TFORF3395-TBX22 | 28 |
| TFORF3545-PIAS1 | 28 |
| TFORF1941-MIER1 | 29 |
| TFORF3002-NFYC | 29 |
| TFORF3170-CNOT3 | 29 |
| TFORF1019-SCRT1 | 30 |
| TFORF2334-CTNNB1 | 30 |
| TFORF2360-ZNF641 | 30 |
| TFORF2816-FOSB | 30 |
| TFORF1416-ZNF215 | 31 |
| TFORF2292-HES7 | 31 |
| TFORF2574-HSF2 | 31 |
| TFORF2743-T | 31 |
| TFORF1133-CARF | 32 |
| TFORF1339-POU3F4 | 32 |
| TFORF2315-RXRB | 32 |
| TFORF2830-IKZF3 | 32 |
| TFORF3405-KCNIP4 | 32 |
| TFORF1025-FOXI2 | 33 |
| TFORF2365-ZNF398 | 33 |
| TFORF3160-SNAI3 | 33 |
| TFORF0276-TCF3 | 34 |
| TFORF0780-GTF2H1 | 34 |
| TFORF1480-TP63 | 34 |
| TFORF2508-PML | 34 |
| TFORF1560-IL18 | 35 |
| TFORF3406-ELOF1 | 35 |
| TFORF3414-NEUROG1 | 35 |
| TFORF0652-NR113 | 36 |
| TFORF1080-NOBOX | 36 |
| TFORF1536-ZBTB44 | 36 |
| TFORF1584-ZNF775 | 36 |
| TFORF0599-ZNF891 | 37 |
| TFORF0923-NR2F2 | 37 |
| TFORF2698-ACTL6A | 37 |
| TFORF3470-ZNF726 | 37 |
| TFORF2108-L3MBTL4 | 38 |
| TFORF2955-HMG20B | 38 |
| TFORF1278-ZNF587 | 39 |
| TFORF1876-CCNT1 | 39 |
| TFORF2019-MYBL2 | 39 |
| TFORF2634-TFDP2 | 39 |
| TFORF2826-IKZF1 | 40 |
| TFORF3421-YBX2 | 40 |
| TFORF0257-TCF4 | 41 |
| TFORF2809-ETV7 | 41 |
| TFORF0021-IRX1 | 42 |
| TFORF1742-ZNF765 | 42 |
| TFORF2523-NRF1 | 42 |
| TFORF2710-DMRTA1 | 42 |
| TFORF0838-ZSCAN18 | 43 |
| TFORF1824-ZNF138 | 43 |
| TFORF2406-NFE2L1 | 43 |
| TFORF3169-GTF2I | 43 |
| TFORF1488-NR6A1 | 44 |
| TFORF2919-NFIB | 44 |
| TFORF2980-FOXP3 | 44 |
| TFORF2999-CDK2 | 44 |
| TFORF3495-CLOCK | 44 |
| TFORF1161-NFKBIL1 | 45 |
| TFORF3510-TCF25 | 45 |
| TFORF0989-KLF9 | 46 |
| TFORF1385-TMF1 | 46 |
| TFORF2613-ZNF276 | 46 |
| TFORF2993-SKP2 | 46 |
| TFORF0898-ING1 | 47 |
| TFORF2507-PML | 47 |
| TFORF3298-EZH2 | 47 |
| TFORF1563-ZNF682 | 48 |
| TFORF1809-TBX19 | 48 |
| TFORF1978-TBX1 | 48 |
| TFORF0188-DMRTC2 | 49 |
| TFORF1329-TFAP2A | 49 |
| TFORF3015-PPARA | 49 |
| TFORF0236-MYCN | 50 |
| TFORF0673-ZNF827 | 50 |
| TFORF0739-ZNF155 | 50 |
| TFORF2949-GCM2 | 50 |
| TFORF0145-PHOX2B | 51 |
| TFORF0530-PAX6 | 51 |
| TFORF1027-RARA | 51 |
| TFORF2827-IKZF1 | 51 |
| TFORF1626-ZNF514 | 52 |
| TFORF3059-FOSL1 | 52 |
| TFORF3060-BACH1 | 52 |
| TFORF3318-TFCP2 | 52 |
| TFORF0957-ZNF497 | 53 |
| TFORF2060-NKX2-1 | 53 |
| TFORF2343-MITF | 53 |
| TFORF3189-ZNF558 | 53 |
| TFORF0484-ZFP92 | 54 |
| TFORF1314-AEBP2 | 54 |
| TFORF1765-ZNF23 | 54 |
| TFORF1945-MIER1 | 54 |
| TFORF2854-FEV | 54 |
| TFORF3004-PCGF2 | 54 |
| TFORF2052-BCL6 | 55 |
| TFORF2383-MBD1 | 55 |
| TFORF2986-MECP2 | 55 |
| TFORF3030-ZNF415 | 55 |
| TFORF0584-SRF | 56 |
| TFORF1853-ZBTB1 | 56 |
| TFORF3064-TERF1 | 56 |
| TFORF0201-RFX3 | 57 |
| TFORF0277-TCF3 | 57 |
| TFORF0629-NR112 | 57 |
| TFORF0913-ZNF835 | 57 |
| TFORF2033-OSR1 | 57 |
| TFORF2577-HSF4 | 57 |
| TFORF2628-NONO | 57 |
| TFORF0052-MAZ | 58 |
| TFORF1702-E2F4 | 58 |
| TFORF2643-PAX3 | 58 |
| TFORF2685-ZMYND11 | 58 |
| TFORF3232-JUNB | 58 |
| TFORF0268-TCF4 | 59 |
| TFORF0688-PRDM10 | 59 |
| TFORF0929-ATOH8 | 59 |
| TFORF1075-NFATC4 | 59 |
| TFORF1251-ZNF133 | 59 |
| TFORF1267-ZNF135 | 59 |
| TFORF1336-POU3F1 | 59 |
| TFORF1570-ZNF680 | 59 |
| TFORF2053-BCL6 | 59 |
| TFORF3013-MEF2A | 59 |
| TFORF3092-NPAS1 | 59 |
| TFORF1946-MIER1 | 60 |
| TFORF2264-ZNF286A | 60 |
| TFORF3036-ATF4 | 60 |
| TFORF0400-ZNF2 | 61 |
| TFORF1240-LBX2 | 61 |
| TFORF2368-MBD4 | 61 |
| TFORF0103-LEUTX | 62 |
| TFORF1774-ZNF500 | 62 |
| TFORF2221-YAP1 | 62 |
| TFORF2690-TEAD2 | 62 |
| TFORF0899-ING1 | 63 |
| TFORF2502-PML | 63 |
| TFORF2578-MEIS3 | 63 |
| TFORF0520-PAX5 | 64 |
| TFORF0535-ZNF584 | 64 |
| TFORF1658-LEF1 | 64 |
| TFORF2462-IRF7 | 64 |
| TFORF3234-VSX1 | 64 |
| TFORF1465-VAV1 | 65 |
| TFORF1856-NR4A2 | 65 |
| TFORF2822-IKZF4 | 65 |
| TFORF2911-NFIC | 65 |
| TFORF0151-THAP5 | 66 |
| TFORF1032-RARG | 66 |
| TFORF1698-E2F6 | 66 |
| TFORF2391-ZNF461 | 66 |
| TFORF2736-PLK4 | 66 |
| TFORF3102-ZNF689 | 66 |
| TFORF3338-HDAC1 | 66 |
| TFORF0075-KCNIP4 | 67 |
| TFORF0115-SMAD3 | 67 |
| TFORF0903-SPDEF | 67 |
| TFORF1360-SP100 | 67 |
| TFORF2189-ZNF410 | 67 |
| TFORF2227-ZNF343 | 67 |
| TFORF2481-MXD4 | 67 |
| TFORF3407-ZNF397 | 67 |
| TFORF0509-TSHZ2 | 68 |
| TFORF0966-ZNF493 | 68 |
| TFORF1380-ZNF787 | 68 |
| TFORF2160-TXK | 68 |
| TFORF2692-TEAD1 | 68 |
| TFORF3086-STAT1 | 68 |
| TFORF3285-FEZF1 | 68 |
| TFORF3439-FOXI1 | 68 |
| TFORF1018-ZNF548 | 69 |
| TFORF1074-NFATC4 | 69 |
| TFORF1373-ZNF143 | 69 |
| TFORF2494-ZBTB18 | 69 |
| TFORF2711-ZNF710 | 69 |
| TFORF3218-FOXN3 | 69 |
| TFORF3262-CEBPE | 69 |
| TFORF0014-ZNF707 | 70 |
| TFORF1989-ZNF530 | 70 |
| TFORF3468-PKNOX1 | 70 |
| TFORF0993-KLF8 | 71 |
| TFORF1275-TPRX1 | 71 |
| TFORF1422-ZNF211 | 71 |
| TFORF1518-FOXD4L5 | 71 |
| TFORF2573-HSF2 | 71 |
| TFORF0343-ETF1 | 72 |
| TFORF0644-NR113 | 72 |
| TFORF0645-NR113 | 72 |
| TFORF0697-BATF2 | 72 |
| TFORF1037-WT1 | 72 |
| TFORF2236-GATA4 | 72 |
| TFORF3392-CNBP | 72 |
| TFORF3498-APEX1 | 72 |
| TFORF0020-IRX6 | 73 |
| TFORF2267-ARNT2 | 73 |
| TFORF2759-L3MBTL1 | 73 |
| TFORF2867-TADA3 | 73 |
| TFORF2906-DACH2 | 73 |
| TFORF3057-YAF2 | 73 |
| TFORF0810-SOHLH1 | 74 |
| TFORF1663-ARID5A | 74 |
| TFORF3024-HEY2 | 74 |
| TFORF1908-TCF7L2 | 75 |
| TFORF2369-MBD4 | 75 |
| TFORF2620-CRX | 75 |
| TFORF2697-ZFP42 | 75 |
| TFORF1285-ZNF257 | 76 |
| TFORF3158-SMARCB1 | 76 |
| TFORF3284-PBX3 | 76 |
| TFORF0170-ZNF587B | 77 |
| TFORF3114-BHLHA15 | 77 |
| TFORF1110-ISX | 78 |
| TFORF2395-ZNF396 | 78 |
| TFORF1112-RCOR3 | 79 |
| TFORF1771-TBP | 79 |
| TFORF0051-MAZ | 80 |
| TFORF0887-ZNF384 | 80 |
| TFORF1172-ZNF157 | 80 |
| TFORF1304-ZMIZ2 | 80 |
| TFORF2836-IKZF3 | 80 |
| TFORF3355-FOXN2 | 80 |
| TFORF0059-CTBP1 | 81 |
| TFORF1568-ZNF683 | 81 |
| TFORF0167-SIX1 | 82 |
| TFORF1503-GABPB1 | 82 |
| TFORF1684-HNF1B | 83 |
| TFORF2110-NR4A1 | 83 |
| TFORF0116-FOXM1 | 84 |
| TFORF0583-ZNF324 | 84 |
| TFORF1688-HNF1A | 84 |
| TFORF1689-HNF1A | 84 |
| TFORF1865-POU2F2 | 84 |
| TFORF0299-CREM | 85 |
| TFORF0638-HOXC6 | 85 |
| TFORF0839-HSFY2 | 85 |
| TFORF2455-IRF3 | 85 |
| TFORF0086-LIN28B | 86 |
| TFORF1772-TBP | 86 |
| TFORF3226-FGF3 | 86 |
| TFORF0587-NMI | 87 |
| TFORF0651-NR113 | 87 |
| TFORF1096-EN2 | 87 |
| TFORF1519-FOXD4L6 | 87 |
| TFORF1557-YAF2 | 88 |
| TFORF1717-LDB1 | 88 |
| TFORF2231-EHF | 88 |
| TFORF3279-LMO1 | 88 |
| TFORF0462-CREB5 | 89 |
| TFORF0746-PIAS3 | 89 |
| TFORF1450-DLX6 | 89 |
| TFORF1660-LEF1 | 89 |
| TFORF2273-PRDM6 | 89 |
| TFORF2397-ZNF397 | 89 |
| TFORF3001-DR1 | 89 |
| TFORF0597-SMARCB1 | 90 |
| TFORF1125-ENO1 | 90 |
| TFORF0235-MYCN | 91 |
| TFORF1713-LDB2 | 91 |
| TFORF3514-PURB | 91 |
| TFORF0628-NR112 | 92 |
| TFORF1042-TSC22D1 | 92 |
| TFORF1169-EBF1 | 92 |
| TFORF3180-RARG | 92 |
| TFORF3247-LEF1 | 92 |
| TFORF0495-EGR2 | 93 |
| TFORF0927-DDB2 | 93 |
| TFORF2492-ZBTB17 | 93 |
| TFORF0832-GTF2F1 | 94 |
| TFORF0880-HOXB3 | 94 |
| TFORF1935-MEIS2 | 94 |
| TFORF2477-OXSR1 | 94 |
| TFORF0527-PAX7 | 95 |
| TFORF1823-ZNF138 | 95 |
| TFORF2050-ATF6B | 95 |
| TFORF2683-ZMYND11 | 95 |
| TFORF0176-RELA | 96 |
| TFORF0469-KAT7 | 96 |
| TFORF0714-ZNF620 | 96 |
| TFORF1087-TULP3 | 96 |
| TFORF1196-ZNF577 | 96 |
| TFORF1324-KLF17 | 96 |
| TFORF1381-ZNF789 | 96 |
| TFORF1818-UHRF1 | 96 |
| TFORF1842-YWHAZ | 96 |
| TFORF1961-CUX1 | 96 |
| TFORF2261-DMAP1 | 96 |
| TFORF2378-MBD1 | 96 |
| TFORF2463-IRF7 | 96 |
| TFORF2614-ZNF275 | 96 |
| TFORF2681-ZMYND11 | 96 |
| TFORF2971-NR1H4 | 96 |
| TFORF3264-ARNTL2 | 96 |
| TFORF0061-CTBP2 | 97 |
| TFORF0097-PSMD14 | 97 |
| TFORF0286-MEF2C | 97 |
| TFORF0422-PBX2 | 97 |
| TFORF2129-ZNF749 | 97 |
| TFORF2243-LITAF | 97 |
| TFORF2588-CERS5 | 97 |
| TFORF2767-ZKSCAN7 | 97 |
| TFORF3537-TSC22D4 | 97 |
| TFORF2210-CREB3L3 | 98 |
| TFORF2570-TGIF1 | 98 |
| TFORF3042-TAF9 | 98 |
| TFORF0777-SHOX | 99 |
| TFORF2054-BCL3 | 99 |
| TFORF3118-ZNF581 | 99 |
| TFORF0411-CTCFL | 100 |
| TFORF1410-RNF2 | 100 |
| TFORF1862-OTP | 100 |
| TFORF2020-MYBL2 | 100 |
| TFORF2872-HMX3 | 100 |
| TFORF0129-SP9 | 101 |
| TFORF0689-HNF4G | 101 |
| TFORF0690-HNF4G | 101 |
| TFORF0691-HNF4A | 101 |
| TFORF0692-HNF4A | 101 |
| TFORF0693-HNF4A | 101 |
| TFORF0694-HNF4A | 101 |
| TFORF0695-HNF4A | 101 |
| TFORF1115-RCOR2 | 101 |
| TFORF1976-TBX1 | 101 |
| TFORF2562-ZNF655 | 101 |
| TFORF2696-ELF5 | 101 |
| TFORF2954-HNF4G | 101 |
| TFORF2957-HNF4A | 101 |
| TFORF3005-SMAD4 | 101 |
| TFORF3542-ZNF720 | 101 |
| TFORF1744-ZNF764 | 102 |
| TFORF1918-RUNX3 | 102 |
| TFORF2563-ZNF655 | 102 |
| TFORF0083-KCNIP2 | 103 |
| TFORF0311-CREM | 103 |
| TFORF1084-SPZ1 | 103 |
| TFORF1653-SPI1 | 103 |
| TFORF2113-SIRT6 | 103 |
| TFORF3502-VENTX | 103 |
| TFORF0143-FXN | 104 |
| TFORF0665-SCX | 104 |
| TFORF1016-ZNF549 | 104 |
| TFORF1498-RBL1 | 104 |
| TFORF1632-ZNF227 | 104 |
| TFORF1703-E2F3 | 104 |
| TFORF3271-EBF3 | 104 |
| TFORF3391-CNBP | 104 |
| TFORF0217-FOXP1 | 105 |
| TFORF0222-FOXP4 | 105 |
| TFORF0655-NR113 | 105 |
| TFORF1920-RUNX1 | 105 |
| TFORF2330-CIZ1 | 105 |
| TFORF2389-ZNF391 | 105 |
| TFORF2505-PML | 105 |
| TFORF1837-EZH2 | 106 |
| TFORF1968-YY1 | 106 |
| TFORF2112-SIRT6 | 106 |
| TFORF2565-ZNF655 | 106 |
| TFORF0709-ZNF629 | 107 |
| TFORF1681-SIM2 | 107 |
| TFORF1773-ZNF501 | 107 |
| TFORF1883-SOX30 | 107 |
| TFORF3034-OTX1 | 107 |
| TFORF1242-LBX1 | 108 |
| TFORF2592-ZNF354B | 108 |
| TFORF3186-GTF2H3 | 108 |
| TFORF0711-ZNF625 | 109 |
| TFORF1049-HKR1 | 109 |
| TFORF1282-SALL4 | 109 |
| TFORF1447-NR5A2 | 109 |
| TFORF1448-NR5A2 | 109 |
| TFORF2641-PAX3 | 109 |
| TFORF2922-NFIA | 109 |
| TFORF3040-ZIM3 | 109 |
| TFORF3131-NR5A2 | 109 |
| TFORF3363-NR5A1 | 109 |
| TFORF0787-REPIN1 | 110 |
| TFORF0958-ZNF496 | 110 |
| TFORF1056-NFATC1 | 110 |
| TFORF1791-FOXE3 | 110 |
| TFORF3164-RUVBL1 | 110 |
| TFORF3211-TADA2B | 110 |
| TFORF3436-TOX4 | 110 |
| TFORF0255-TCF4 | 111 |
| TFORF1706-E2F1 | 111 |
| TFORF2312-ETV3 | 111 |
| TFORF3196-ZIC1 | 111 |
| TFORF0888-ZNF384 | 112 |
| TFORF1185-MAP3K7 | 112 |
| TFORF2424-RBPJL | 112 |
| TFORF0184-SHOX2 | 113 |
| TFORF0548-PAX8 | 113 |
| TFORF2504-PML | 113 |
| TFORF0948-EMX2 | 114 |
| TFORF3457-IRF9 | 114 |
| TFORF3534-BORCS8-MEF2B | 114 |
| TFORF0491-ZSCAN1 | 115 |
| TFORF1014-ZNF540 | 115 |
| TFORF2366-MBD4 | 115 |
| TFORF0316-MEF2D | 116 |
| TFORF1303-ZMIZ2 | 116 |
| TFORF2198-MLXIPL | 116 |
| TFORF2799-TAF6 | 116 |
| TFORF3031-STRAP | 116 |
| TFORF3336-ZBTB37 | 116 |
| TFORF2190-ZNF410 | 117 |
| TFORF2896-MTA2 | 117 |
| TFORF2908-NFIC | 117 |
| TFORF3246-HOXC9 | 117 |
| TFORF0044-ZNF672 | 118 |
| TFORF0423-PBX3 | 118 |
| TFORF2622-CRTC1 | 118 |
| TFORF3356-NRF1 | 118 |
| TFORF0265-TCF4 | 119 |
| TFORF0334-ZNF331 | 119 |
| TFORF1257-TP53 | 119 |
| TFORF1817-UHRF1 | 119 |
| TFORF2351-ZNF17 | 119 |
| TFORF2650-ZNF586 | 119 |
| TFORF2724-ZNF74 | 119 |
| TFORF3352-HESX1 | 119 |
| TFORF0001-HIF3A | 120 |
| TFORF0841-ZSCAN10 | 120 |
| TFORF1057-NFATC1 | 120 |
| TFORF1101-GTF2IRD1 | 120 |
| TFORF1122-HNRNPAB | 120 |
| TFORF1898-PATZ1 | 120 |
| TFORF1904-TCF7L2 | 120 |
| TFORF2143-ZNF525 | 120 |
| TFORF2230-EHF | 120 |
| TFORF2335-CTNNB1 | 120 |
| TFORF2558-TLX1 | 120 |
| TFORF2568-TGIF1 | 120 |
| TFORF2930-NFIX | 120 |
| TFORF2984-MAP3K7 | 120 |
| TFORF3084-ZFP36L1 | 120 |
| TFORF3455-ZNF513 | 120 |
| TFORF2233-SMARCD1 | 121 |
| TFORF2524-ERCC8 | 121 |
| TFORF0982-KLF6 | 122 |
| TFORF1721-DMRT2 | 122 |
| TFORF2408-FOXF1 | 122 |
| TFORF3428-TRIM27 | 122 |
| TFORF0213-FOXP1 | 123 |
| TFORF0844-ZSCAN12 | 123 |
| TFORF1088-TULP3 | 123 |
| TFORF0420-SKIL | 124 |
| TFORF1627-FOXJ3 | 124 |
| TFORF2997-GATA2 | 124 |
| TFORF1097-HSFX1 | 125 |
| TFORF1344-FEZF2 | 125 |
| TFORF1526-MXI1 | 125 |
| TFORF1715-LDB2 | 125 |
| TFORF2195-MLXIPL | 125 |
| TFORF3134-TFE3 | 125 |
| TFORF3172-STAT6 | 125 |
| TFORF3174-ARID5A | 125 |
| TFORF1386-ZNF362 | 126 |
| TFORF1781-ZNF506 | 126 |
| TFORF2232-EHF | 126 |
| TFORF2991-ID2 | 126 |
| TFORF2843-RHOXF2B | 127 |
| TFORF2881-XBP1 | 127 |
| TFORF2916-NFIB | 127 |
| TFORF3144-MIER2 | 127 |
| TFORF3311-CREB3 | 127 |
| TFORF3314-SMAD9 | 127 |
| TFORF0897-ING1 | 128 |
| TFORF1010-ZNF547 | 128 |
| TFORF1217-PRRX1 | 128 |
| TFORF1260-TP53 | 128 |
| TFORF2317-ETV4 | 128 |
| TFORF0605-PUF60 | 129 |
| TFORF1977-TBX1 | 129 |
| TFORF2005-ZNF664 | 129 |
| TFORF3032-SIRT6 | 129 |
| TFORF1511-STAT5A | 130 |
| TFORF1561-ZNF688 | 130 |
| TFORF3528-NME2 | 130 |
| TFORF0306-CREM | 131 |
| TFORF0394-ZNF7 | 131 |
| TFORF0874-HOXB9 | 131 |
| TFORF1044-TSC22D3 | 131 |
| TFORF1955-ESR2 | 131 |
| TFORF2162-GRHL1 | 131 |
| TFORF2404-NFE2L1 | 131 |
| TFORF2423-RBPJL | 131 |
| TFORF3308-GTF2A1L | 131 |
| TFORF3364-ZNF232 | 131 |
| TFORF3416-ZNF410 | 131 |
| TFORF0533-PAX1 | 132 |
| TFORF0666-TGFB111 | 132 |
| TFORF1366-ISL2 | 132 |
| TFORF2252-ZNF630 | 132 |
| TFORF2631-INSM1 | 132 |
| TFORF2689-TEAD2 | 132 |
| TFORF3306-THAP1 | 132 |
| TFORF3384-HOXC10 | 132 |
| TFORF3433-DEK | 132 |
| TFORF3506-LCOR | 132 |
| TFORF1291-SP140L | 133 |
| TFORF2829-IKZF3 | 133 |
| TFORF1039-TSC22D1 | 134 |
| TFORF1387-ZNF367 | 134 |
| TFORF0362-TCEB2 | 135 |
| TFORF2131-ZGLP1 | 135 |
| TFORF2354-ZNF12 | 135 |
| TFORF2444-SMARCE1 | 135 |
| TFORF3016-MLX | 135 |
| TFORF3466-SIM2 | 135 |
| TFORF3489-IKZF1 | 135 |
| TFORF0516-PAX5 | 136 |
| TFORF2319-TAF1B | 136 |
| TFORF2654-ARNTL2 | 136 |
| TFORF0426-PBX3 | 137 |
| TFORF1126-THAP3 | 137 |
| TFORF1221-HELT | 137 |
| TFORF2152-BCL11A | 137 |
| TFORF2569-TGIF1 | 137 |
| TFORF1649-REL | 138 |
| TFORF2350-ZNF16 | 138 |
| TFORF2576-HSF4 | 138 |
| TFORF2800-TAF5 | 138 |
| TFORF3141-ALX3 | 138 |
| TFORF3147-ZBTB43 | 138 |
| TFORF1884-ZIC2 | 139 |
| TFORF3266-ZNF213 | 139 |
| TFORF3482-SOX2 | 139 |
| TFORF1099-UBP1 | 140 |
| TFORF3494-OSR2 | 140 |
| TFORF0826-LHX9 | 141 |
| TFORF1516-ZNF737 | 141 |
| TFORF1664-ARID5A | 141 |
| TFORF1878-CCNT2 | 141 |
| TFORF2035-OSR2 | 141 |
| TFORF3259-MEF2D | 141 |
| TFORF0279-ZNF776 | 142 |
| TFORF0985-KLF4 | 142 |
| TFORF1246-ZNF133 | 142 |
| TFORF1298-ZFP64 | 142 |
| TFORF1357-ILF3 | 142 |
| TFORF1725-ZNF691 | 142 |
| TFORF3154-ZFP2 | 142 |
| TFORF3411-CRTC2 | 142 |
| TFORF0589-FOXO1 | 143 |
| TFORF0868-NR1H3 | 143 |
| TFORF1123-HNRNPAB | 143 |
| TFORF1860-NR4A3 | 143 |
| TFORF1960-CUX1 | 143 |
| TFORF2186-ZNF414 | 143 |
| TFORF2466-IRF5 | 143 |
| TFORF2635-TFDP2 | 143 |
| TFORF2798-TAF6 | 143 |
| TFORF2915-NFIB | 143 |
| TFORF2933-HOXD3 | 143 |
| TFORF3230-FOXJ1 | 143 |
| TFORF3386-IRF4 | 143 |
| TFORF0192-RFX4 | 144 |
| TFORF0325-LCORL | 144 |
| TFORF1609-ZZZ3 | 144 |
| TFORF1962-CUX1 | 144 |
| TFORF3138-PPARG | 144 |
| TFORF3345-LMO3 | 144 |
| TFORF3480-ZNF785 | 144 |
| TFORF0611-RBPJ | 145 |
| TFORF0618-ZSCAN26 | 145 |
| TFORF2204-MYNN | 145 |
| TFORF2239-GATA5 | 145 |
| TFORF2808-ETV7 | 145 |
| TFORF0240-MYCL | 146 |
| TFORF0505-CDK2 | 146 |
| TFORF0766-ZNF552 | 146 |
| TFORF1215-NME2 | 146 |
| TFORF1836-EZH2 | 146 |
| TFORF2142-ZNF525 | 146 |
| TFORF2664-ZBED1 | 146 |
| TFORF3021-DLX4 | 146 |
| TFORF3332-PUF60 | 146 |
| TFORF3540-TAL2 | 146 |
| TFORF0082-KCNIP2 | 147 |
| TFORF0085-KCNIP2 | 147 |
| TFORF0267-TCF4 | 147 |
| TFORF0546-ZFAT | 147 |
| TFORF0840-HSFY2 | 147 |
| TFORF1382-ZNF789 | 147 |
| TFORF1850-YWHAE | 147 |
| TFORF2414-ZNF268 | 147 |
| TFORF2585-CERS6 | 147 |
| TFORF2716-SLC2A4RG | 147 |
| TFORF1295-ZFP64 | 148 |
| TFORF1881-SOX30 | 148 |
| TFORF0453-THRA | 149 |
| TFORF0857-VSX1 | 149 |
| TFORF2281-MYBBP1A | 149 |
| TFORF2567-ZNF654 | 149 |
| TFORF2656-STAT3 | 149 |
| TFORF3053-ZBTB44 | 149 |
| TFORF0488-ZFP90 | 150 |
| TFORF0755-ZNF319 | 150 |
| TFORF1045-TSC22D2 | 150 |
| TFORF1238-HOMEZ | 150 |
| TFORF1458-TFEB | 150 |
| TFORF1761-ZNF24 | 150 |
| TFORF2077-MYB | 150 |
| TFORF2270-PSAP | 150 |
| TFORF0003-HIF3A | 151 |
| TFORF0502-NFE4 | 151 |
| TFORF2357-ZNF649 | 151 |
| TFORF2817-FOXD4L3 | 151 |
| TFORF0153-THAP6 | 152 |
| TFORF0475-SP140 | 152 |
| TFORF1163-NFKBIL1 | 152 |
| TFORF1657-SPIB | 152 |
| TFORF1743-ZNF764 | 152 |
| TFORF3121-ZNF341 | 152 |
| TFORF3299-MEIS2 | 152 |
| TFORF3330-TBX20 | 152 |
| TFORF2229-ZNF730 | 153 |
| TFORF2361-ZNF641 | 153 |
| TFORF0468-KAT7 | 154 |
| TFORF0633-VPS72 | 154 |
| TFORF0650-NR113 | 154 |
| TFORF0799-ATF3 | 154 |
| TFORF1782-ZNF239 | 154 |
| TFORF1910-TCF7L2 | 154 |
| TFORF2015-HMGXB4 | 154 |
| TFORF2046-ARID3B | 154 |
| TFORF3354-MXD1 | 154 |
| TFORF0037-SP5 | 155 |
| TFORF0987-KLF2 | 155 |
| TFORF1432-NR3C1 | 155 |
| TFORF1453-DLX2 | 155 |
| TFORF2006-CAPN15 | 155 |
| TFORF2589-ZNF354A | 155 |
| TFORF2719-ZNF79 | 155 |
| TFORF2868-TADA3 | 155 |
| TFORF3125-ZNF300 | 155 |
| TFORF0609-MAFF | 156 |
| TFORF2950-MAFB | 156 |
| TFORF3198-OVOL2 | 156 |
| TFORF3543-CSDC2 | 156 |
| TFORF0358-ZNF33A | 157 |
| TFORF0983-KLF5 | 157 |
| TFORF0984-KLF5 | 157 |
| TFORF0988-KLF1 | 157 |
| TFORF1371-ZNF140 | 157 |
| TFORF2660-STAT1 | 157 |
| TFORF2871-HMX1 | 157 |
| TFORF3130-IRF8 | 157 |
| TFORF3412-EGR1 | 157 |
| TFORF3444-KCNIP2 | 157 |
| TFORF3463-ETV7 | 157 |
| TFORF0455-TAF4B | 158 |
| TFORF0511-TSHZ1 | 158 |
| TFORF1893-AHRR | 158 |
| TFORF0208-FOXP2 | 159 |
| TFORF0209-FOXP2 | 159 |
| TFORF0210-FOXP2 | 159 |
| TFORF0215-FOXP1 | 159 |
| TFORF0216-FOXP1 | 159 |
| TFORF0220-FOXP4 | 159 |
| TFORF0221-FOXP4 | 159 |
| TFORF1365-ISL1 | 159 |
| TFORF3181-PATZ1 | 159 |
| TFORF0214-FOXP1 | 160 |
| TFORF0549-PAX8 | 160 |
| TFORF0744-PIAS2 | 160 |
| TFORF0997-ZNF302 | 160 |
| TFORF1093-ZBTB8B | 160 |
| TFORF1413-ZNF561 | 160 |
| TFORF2057-ARGFX | 160 |
| TFORF2308-ETV1 | 160 |
| TFORF2309-ETV1 | 160 |
| TFORF2310-ETV1 | 160 |
| TFORF0624-NKRF | 161 |
| TFORF1780-ZNF506 | 161 |
| TFORF2401-NFE2L3 | 161 |
| TFORF3334-SP100 | 161 |
| TFORF1631-ZNF226 | 162 |
| TFORF1924-ZBTB21 | 162 |
| TFORF2469-IRF8 | 162 |
| TFORF2514-PDCD2 | 162 |
| TFORF2649-ZNF586 | 162 |
| TFORF2752-ZNF444 | 162 |
| TFORF3145-ZNF660 | 162 |
| TFORF3419-ZNF562 | 162 |
| TFORF2172-ZNF419 | 163 |
| TFORF2495-ZBTB18 | 163 |
| TFORF2572-ZNF652 | 163 |
| TFORF2804-ETV2 | 163 |
| TFORF2837-IKZF3 | 163 |
| TFORF2994-SKP2 | 163 |
| TFORF3203-ZNF526 | 163 |
| TFORF1804-ZNF347 | 164 |
| TFORF1808-TBX18 | 164 |
| TFORF2503-PML | 164 |
| TFORF2636-TFDP2 | 164 |
| TFORF2677-GABPA | 164 |
| TFORF2703-ESRRA | 164 |
| TFORF2943-ESRRG | 164 |
| TFORF3515-HDGF | 164 |
| TFORF0150-THAP5 | 165 |
| TFORF0263-TCF4 | 165 |
| TFORF0312-CREM | 165 |
| TFORF0724-NFKB2 | 165 |
| TFORF0959-ERF | 165 |
| TFORF1678-RUNX1T1 | 165 |
| TFORF2910-NFIC | 165 |
| TFORF2920-NFIB | 165 |
| TFORF2923-NFIA | 165 |
| TFORF1549-TAZ | 166 |
| TFORF2030-TGIF2LX | 166 |
| TFORF2130-PFDN5 | 166 |
| TFORF2170-BHLHE22 | 166 |
| TFORF2194-TFAM | 166 |
| TFORF2277-PRDM2 | 166 |
| TFORF2413-ZNF268 | 166 |
| TFORF2939-HOXA6 | 166 |
| TFORF0078-KCNIP4 | 167 |
| TFORF0271-TCF7 | 167 |
| TFORF0802-ATF2 | 167 |
| TFORF0811-CDCA7L | 167 |
| TFORF0858-VSX1 | 167 |
| TFORF1347-RPA3 | 167 |
| TFORF1434-NFKBID | 167 |
| TFORF1507-HEY1 | 167 |
| TFORF2120-HOPX | 167 |
| TFORF2156-RHOXF2 | 167 |
| TFORF3216-ATF2 | 167 |
| TFORF3422-HES6 | 167 |
| TFORF3459-HMGB2 | 167 |
| TFORF0080-KCNIP3 | 168 |
| TFORF0379-SMARCA2 | 168 |
| TFORF0636-HOXC5 | 168 |
| TFORF0931-MECP2 | 168 |
| TFORF1843-ALX1 | 168 |
| TFORF2096-ZKSCAN1 | 168 |
| TFORF0107-TRERF1 | 169 |
| TFORF0315-CREM | 169 |
| TFORF0622-ZSCAN23 | 169 |
| TFORF0631-TERF1 | 169 |
| TFORF1107-ZNF37A | 169 |
| TFORF1472-TOX2 | 169 |
| TFORF1520-NR2E1 | 169 |
| TFORF1832-MTERF1 | 169 |
| TFORF1965-PPARD | 169 |
| TFORF2021-NPAS4 | 169 |
| TFORF2049-PURA | 169 |
| TFORF2191-RAX2 | 169 |
| TFORF2208-CREB3L2 | 169 |
| TFORF2557-ZNF69 | 169 |
| TFORF2903-DACH1 | 169 |
| TFORF3006-ZBTB12 | 169 |
| TFORF3049-CDK1 | 169 |
| TFORF3238-GTF2A1 | 169 |
| TFORF3244-ZNF483 | 169 |
| TFORF0300-CREM | 170 |
| TFORF0305-CREM | 170 |
| TFORF1430-NFKBIB | 170 |
| TFORF1820-ZNF138 | 170 |
| TFORF2023-NPAS3 | 170 |
| TFORF2314-RXRB | 170 |
| TFORF2359-ZNF644 | 170 |
| TFORF2384-MBD3 | 170 |
| TFORF2687-ZMYND11 | 170 |
| TFORF2704-ESRRA | 170 |
| TFORF2860-ID1 | 170 |
| TFORF3035-NFKBIB | 170 |
| TFORF3051-CDK1 | 170 |
| TFORF3316-MED21 | 170 |
| TFORF3373-ZNF101 | 170 |
| TFORF3396-TARBP2 | 170 |
| TFORF3410-ZKSCAN4 | 170 |
| TFORF0018-IRX4 | 171 |
| TFORF0099-HLF | 171 |
| TFORF0111-SMAD7 | 171 |
| TFORF0148-THAP4 | 171 |
| TFORF0211-FOXP3 | 171 |
| TFORF0280-IGFBP1 | 171 |
| TFORF0307-CREM | 171 |
| TFORF0310-CREM | 171 |
| TFORF0314-CREM | 171 |
| TFORF0653-NR113 | 171 |
| TFORF1052-HKR1 | 171 |
| TFORF1144-HOXA7 | 171 |
| TFORF1200-SETDB1 | 171 |
| TFORF1214-FOXH1 | 171 |
| TFORF1445-GTF2H3 | 171 |
| TFORF1822-ZNF138 | 171 |
| TFORF1847-ETS1 | 171 |
| TFORF1861-DRGX | 171 |
| TFORF1900-PATZ1 | 171 |
| TFORF2305-ETV1 | 171 |
| TFORF2321-TAF1A | 171 |
| TFORF2464-IRF6 | 171 |
| TFORF2555-SUZ12 | 171 |
| TFORF2670-NEUROD2 | 171 |
| TFORF2672-CHURC1 | 171 |
| TFORF2673-CHURC1 | 171 |
| TFORF2694-ELF5 | 171 |
| TFORF2695-ELF5 | 171 |
| TFORF2701-ESRRG | 171 |
| TFORF2802-ETV2 | 171 |
| TFORF2844-HNRNPK | 171 |
| TFORF3258-TAF12 | 171 |
| TFORF0178-HDGF | 172 |
| TFORF0205-DBP | 172 |
| TFORF0225-ZNF593 | 172 |
| TFORF0351-FOXL2 | 172 |
| TFORF0615-ZSCAN26 | 172 |
| TFORF0792-TARBP2 | 172 |
| TFORF0876-GLMP | 172 |
| TFORF1383-ZNF788 | 172 |
| TFORF2450-MLLT10 | 172 |
| TFORF2646-PAX3 | 172 |
| TFORF2663-STOX1 | 172 |
| TFORF3447-MYCBP | 172 |
| TFORF3460-PSMB1 | 172 |
| TFORF3513-GLMP | 172 |
| TFORF0539-ZNF589 | 173 |
| TFORF0717-TEF | 173 |
| TFORF0733-PROX2 | 173 |
| TFORF0875-GLMP | 173 |
| TFORF0932-ZNF80 | 173 |
| TFORF1222-ZNF483 | 173 |
| TFORF1259-TP53 | 173 |
| TFORF1671-TP73 | 173 |
| TFORF1902-TCF7L2 | 173 |
| TFORF2853-HMGA2 | 173 |
| TFORF0592-FOXO4 | 174 |
| TFORF0960-ERG | 174 |
| TFORF1091-ZBTB8A | 174 |
| TFORF1851-ZBTB3 | 174 |
| TFORF1882-SOX30 | 174 |
| TFORF2337-ZNF18 | 174 |
| TFORF3257-ZNF800 | 174 |
| TFORF0189-RFX8 | 175 |
| TFORF0203-ELK1 | 175 |
| TFORF2674-CHURC1 | 175 |
| TFORF3490-TCF23 | 175 |
| TFORF1029-RARB | 176 |
| TFORF1933-MEIS2 | 176 |
| TFORF1194-ZNF575 | 177 |
| TFORF1449-DLX4 | 177 |
| TFORF2580-MEIS3 | 177 |
| TFORF0439-ZSCAN30 | 178 |
| TFORF0541-GCFC2 | 178 |
| TFORF0834-HSFY1 | 178 |
| TFORF0870-NR1H3 | 178 |
| TFORF1258-TP53 | 178 |
| TFORF2286-HES4 | 178 |
| TFORF0506-CDK2 | 179 |
| TFORF1957-ESR2 | 179 |
| TFORF1966-PPARD | 179 |
| TFORF2177-ZNF419 | 179 |
| TFORF0047-MAX | 180 |
| TFORF0207-FOXP2 | 180 |
| TFORF1149-HOXA1 | 180 |
| TFORF1226-DUX4 | 180 |
| TFORF2552-BID | 180 |
| TFORF0100-NOV | 181 |
| TFORF0146-THAP1 | 181 |
| TFORF0494-ZSCAN2 | 181 |
| TFORF0642-NR113 | 181 |
| TFORF0944-LMO3 | 181 |
| TFORF1028-RARA | 181 |
| TFORF1963-CUX1 | 181 |
| TFORF2485-MXD1 | 181 |
| TFORF0364-TCEB1 | 182 |
| TFORF0522-PAX5 | 182 |
| TFORF0830-SLC45A2 | 182 |
| TFORF1170-MLX | 182 |
| TFORF2141-ZNF525 | 182 |
| TFORF2291-HES6 | 182 |
| TFORF2662-STOX1 | 182 |
| TFORF0297-CREM | 183 |
| TFORF0604-PUF60 | 183 |
| TFORF1544-ZNF160 | 183 |
| TFORF1630-ZNF226 | 183 |
| TFORF1668-TP73 | 183 |
| TFORF1756-ZNF189 | 183 |
| TFORF2171-BHLHE23 | 183 |
| TFORF2216-NOTO | 183 |
| TFORF2792-ZNF282 | 183 |
| TFORF3063-FOXP1 | 183 |
| TFORF0177-HDGF | 184 |
| TFORF0451-THRA | 184 |
| TFORF0890-ING4 | 184 |
| TFORF1548-TAZ | 184 |
| TFORF1996-ZNF248 | 184 |
| TFORF0133-ZNF780A | 185 |
| TFORF0855-VSX1 | 185 |
| TFORF0936-SS18 | 185 |
| TFORF1218-PRRX1 | 185 |
| TFORF1262-TP53 | 185 |
| TFORF1527-MXI1 | 185 |
| TFORF1709-ZFP41 | 185 |
| TFORF2296-TAF11 | 185 |
| TFORF3389-ZNF580 | 185 |
| TFORF0867-NR1H2 | 186 |
| TFORF0889-ZNF384 | 186 |
| TFORF1327-KLF14 | 186 |
| TFORF1452-DLX1 | 186 |
| TFORF1762-ZNF24 | 186 |
| TFORF3239-HOXB5 | 186 |
| TFORF0060-CTBP2 | 187 |
| TFORF0528-PAX6 | 187 |
| TFORF0829-SLC45A2 | 187 |
| TFORF1320-KLF11 | 187 |
| TFORF1591-CTCF | 187 |
| TFORF1600-ZNF195 | 187 |
| TFORF2176-ZNF419 | 187 |
| TFORF1035-WT1 | 188 |
| TFORF1929-XRCC4 | 188 |
| TFORF2935-MAFK | 188 |
| TFORF3050-CDK1 | 188 |
| TFORF3228-MAX | 188 |
| TFORF0034-SP2 | 189 |
| TFORF2483-CBX2 | 189 |
| TFORF2835-IKZF3 | 189 |
| TFORF0424-PBX3 | 190 |
| TFORF2297-TAF10 | 190 |
| TFORF3148-NFE2 | 190 |
| TFORF3383-NFKBID | 190 |
| TFORF0144-PHOX2A | 191 |
| TFORF0194-RFX6 | 191 |
| TFORF0723-NFKB1 | 192 |
| TFORF0747-PIAS1 | 192 |
| TFORF1254-TP53 | 192 |
| TFORF1456-TFEC | 192 |
| TFORF1673-TP73 | 192 |
| TFORF1733-ZNF169 | 192 |
| TFORF1795-CNBP | 192 |
| TFORF2000-MYEF2 | 192 |
| TFORF2972-NR1H4 | 192 |
| TFORF3191-ZNF436 | 192 |
| TFORF0319-MSLN | 193 |
| TFORF0873-HOXB8 | 193 |
| TFORF0906-DNAJC2 | 193 |
| TFORF0954-CDIP1 | 193 |
| TFORF1274-PKNOX1 | 193 |
| TFORF1476-VAX1 | 193 |
| TFORF1558-YAF2 | 193 |
| TFORF1841-HBP1 | 193 |
| TFORF2018-PITX3 | 193 |
| TFORF2642-PAX3 | 193 |
| TFORF2894-MTA1 | 193 |
| TFORF3105-CCNH | 193 |
| TFORF3107-RARA | 193 |
| TFORF3128-IRF5 | 193 |
| TFORF3370-MYBL1 | 193 |
| TFORF3375-TGIF2 | 193 |
| TFORF3461-ETV1 | 193 |
| TFORF3529-VDR | 193 |
| TFORF3544-NR2E1 | 193 |
| TFORF0454-TAF4B | 194 |
| TFORF0607-CREBL2 | 194 |
| TFORF0740-JDP2 | 194 |
| TFORF0964-ERG | 194 |
| TFORF1118-SKP2 | 194 |
| TFORF1431-ZNF219 | 194 |
| TFORF1734-RUVBL1 | 194 |
| TFORF2192-RFXANK | 194 |
| TFORF2813-FOXD4L1 | 194 |
| TFORF2987-RORA | 194 |
| TFORF3176-ZNF544 | 194 |
| TFORF3339-HDAC3 | 194 |
| TFORF3348-PITX2 | 194 |
| TFORF3368-FOXR1 | 194 |
| TFORF3445-PTTG1 | 194 |
| TFORF3522-POU6F1 | 194 |
| TFORF0383-SMARCA2 | 195 |
| TFORF0817-LHX3 | 195 |
| TFORF1024-FOXI3 | 195 |
| TFORF1050-HKR1 | 195 |
| TFORF1655-SPIB | 195 |
| TFORF2007-PREB | 195 |
| TFORF2016-PITX2 | 195 |
| TFORF2082-IGHMBP2 | 195 |
| TFORF2118-TBPL1 | 195 |
| TFORF2217-YAP1 | 195 |
| TFORF2470-PDX1 | 195 |
| TFORF3261-OVOL1 | 195 |
| TFORF0344-ETF1 | 196 |
| TFORF0371-NFYC | 196 |
| TFORF0643-NR113 | 196 |
| TFORF0871-NR1H3 | 196 |
| TFORF1043-TSC22D3 | 196 |
| TFORF1712-DPRX | 196 |
| TFORF1747-ZNF761 | 196 |
| TFORF1979-TBX6 | 196 |
| TFORF1985-ZNF534 | 196 |
| TFORF2212-CREB3L3 | 196 |
| TFORF2393-ZNF394 | 196 |
| TFORF2564-ZNF655 | 196 |
| TFORF3076-GTF2A2 | 196 |
| TFORF0159-EWSR1 | 197 |
| TFORF1389-PMS1 | 197 |
| TFORF1700-E2F5 | 197 |
| TFORF2045-ARID3B | 197 |
| TFORF2593-BHLHE40 | 197 |
| TFORF2637-TFDP2 | 197 |
| TFORF2807-ETV7 | 197 |
| TFORF0229-ZNF595 | 198 |
| TFORF0827-LHX9 | 198 |
| TFORF1666-TP73 | 198 |
| TFORF1701-E2F5 | 198 |
| TFORF1875-CCNT1 | 198 |
| TFORF2024-NPAS3 | 198 |
| TFORF2206-POU2F3 | 198 |
| TFORF2257-ARNTL | 198 |
| TFORF2417-ZNF268 | 198 |
| TFORF3243-ZNF267 | 198 |
| TFORF0510-TSHZ3 | 199 |
| TFORF0731-PROX1 | 199 |
| TFORF0737-ZNF480 | 199 |
| TFORF1925-ZBTB21 | 199 |
| TFORF2506-PML | 199 |
| TFORF2889-BRPF1 | 199 |
| TFORF0141-POU5F1B | 200 |
| TFORF0356-ZNF33A | 200 |
| TFORF2547-CEBPD | 200 |
| TFORF2688-TEAD3 | 200 |
| TFORF0162-PES1 | 201 |
| TFORF0366-NFYC | 201 |
| TFORF0435-ZSCAN32 | 201 |
| TFORF0463-HOXD1 | 201 |
| TFORF0586-SRY | 201 |
| TFORF1401-ZNF567 | 201 |
| TFORF1628-FOXJ3 | 201 |
| TFORF1998-ZNF248 | 201 |
| TFORF2147-ZNF528 | 201 |
| TFORF2434-ZNF280D | 201 |
| TFORF2918-NFIB | 201 |
| TFORF3215-ZNF248 | 201 |
| TFORF3387-ZNF586 | 201 |
| TFORF0293-MEF2A | 202 |
| TFORF0699-BATF2 | 202 |
| TFORF0710-ZNF624 | 202 |
| TFORF1794-CNBP | 202 |
| TFORF2948-HOXB6 | 202 |
| TFORF2968-NR0B1 | 202 |
| TFORF2995-SKP2 | 202 |
| TFORF3485-ATF2 | 202 |
| TFORF0933-ZNF85 | 203 |
| TFORF2304-CIITA | 203 |
| TFORF2362-ZNF641 | 203 |
| TFORF2849-HMGA2 | 203 |
| TFORF3240-POU2AF1 | 203 |
| TFORF3319-ZSCAN5A | 203 |
| TFORF0239-MYCL | 204 |
| TFORF0372-NFYB | 204 |
| TFORF0877-GLMP | 204 |
| TFORF1026-RARA | 204 |
| TFORF2248-TCF12 | 204 |
| TFORF2313-ETV3 | 204 |
| TFORF2969-PAX8 | 204 |
| TFORF3532-LIN28B | 204 |
| TFORF0529-PAX6 | 205 |
| TFORF1195-ZNF576 | 205 |
| TFORF2017-PITX2 | 205 |
| TFORF3014-CEBPG | 205 |
| TFORF0071-CSDE1 | 206 |
| TFORF0363-TCEB2 | 206 |
| TFORF0402-ZNF3 | 206 |
| TFORF0425-PBX3 | 206 |
| TFORF1792-CNBP | 206 |
| TFORF2100-SOHLH2 | 206 |
| TFORF2454-IRF3 | 206 |
| TFORF2550-BID | 206 |
| TFORF0234-MNX1 | 207 |
| TFORF0798-ATF3 | 207 |
| TFORF1611-GSX2 | 207 |
| TFORF1683-TWIST1 | 207 |
| TFORF1728-ZNF695 | 207 |
| TFORF2183-ZNF415 | 207 |
| TFORF2579-MEIS3 | 207 |
| TFORF2814-FOSB | 207 |
| TFORF0284-MEF2C | 208 |
| TFORF0298-CREM | 208 |
| TFORF0417-SULT2A1 | 208 |
| TFORF0848-YBX3 | 208 |
| TFORF1364-ELOF1 | 208 |
| TFORF1446-GTF2H3 | 208 |
| TFORF1840-GTF2A1 | 208 |
| TFORF2211-CREB3L3 | 208 |
| TFORF2419-ZNF268 | 208 |
| TFORF2597-RORA | 208 |
| TFORF3328-ZSCAN16 | 208 |
| TFORF0978-KLF7 | 209 |
| TFORF1574-ZNF174 | 209 |
| TFORF1691-PGR | 209 |
| TFORF2525-ERCC2 | 209 |
| TFORF3173-STAT6 | 209 |
| TFORF0079-KCNIP3 | 210 |
| TFORF0087-LIN28A | 210 |
| TFORF0261-TCF4 | 210 |
| TFORF0272-TCF7 | 210 |
| TFORF0373-NFYA | 210 |
| TFORF0669-ZNF821 | 210 |
| TFORF0872-HOXB8 | 210 |
| TFORF1145-CDK1 | 210 |
| TFORF1486-NANOG | 210 |
| TFORF1589-ZNF773 | 210 |
| TFORF1767-ZNF22 | 210 |
| TFORF2223-YAP1 | 210 |
| TFORF2285-HES3 | 210 |
| TFORF2448-GBX2 | 210 |
| TFORF2632-TFDP3 | 210 |
| TFORF2825-IKZF1 | 210 |
| TFORF3150-ZNF254 | 210 |
| TFORF3245-MAFG | 210 |
| TFORF1177-ZNF793 | 211 |
| TFORF2812-ETV7 | 211 |
| TFORF3089-ZSCAN9 | 211 |
| TFORF3278-MEOX1 | 211 |
| TFORF0128-SP8 | 212 |
| TFORF0365-TCEB1 | 212 |
| TFORF1022-SCRT2 | 212 |
| TFORF2215-CREB3L4 | 212 |
| TFORF0081-KCNIP2 | 213 |
| TFORF0238-MYCL | 213 |
| TFORF0503-RNF138 | 213 |
| TFORF0600-MZF1 | 213 |
| TFORF0741-JDP2 | 213 |
| TFORF1040-TSC22D1 | 213 |
| TFORF2114-SIRT6 | 213 |
| TFORF2144-ZNF525 | 213 |
| TFORF2294-TAF13 | 213 |
| TFORF2418-ZNF268 | 213 |
| TFORF2516-PDCD2 | 213 |
| TFORF2561-ZNF655 | 213 |
| TFORF3535-MLLT10 | 213 |
| TFORF0048-MAX | 214 |
| TFORF0076-KCNIP4 | 214 |
| TFORF0131-HMGB4 | 214 |
| TFORF0244-SUB1 | 214 |
| TFORF0252-CAMTA1 | 214 |
| TFORF0304-CREM | 214 |
| TFORF0374-NFYA | 214 |
| TFORF0399-ZNF2 | 214 |
| TFORF0647-NR113 | 214 |
| TFORF0771-MSX2 | 214 |
| TFORF0801-ATF3 | 214 |
| TFORF0942-LMO1 | 214 |
| TFORF0992-KLF8 | 214 |
| TFORF1219-PRRX2 | 214 |
| TFORF1494-FOSL1 | 214 |
| TFORF1556-YAF2 | 214 |
| TFORF2121-HOPX | 214 |
| TFORF2250-TCF19 | 214 |
| TFORF2253-HMGN3 | 214 |
| TFORF2586-CERS4 | 214 |
| TFORF2857-ID4 | 214 |
| TFORF3000-E2F6 | 214 |
| TFORF3142-CDX4 | 214 |
| TFORF3207-HMGN2 | 214 |
| TFORF3274-HMGA1 | 214 |
| TFORF3294-ZNF784 | 214 |
| TFORF3530-SSRP1 | 214 |
| TFORF0084-KCNIP2 | 215 |
| TFORF0183-SHOX2 | 215 |
| TFORF0349-ZNF48 | 215 |
| TFORF0938-SS18 | 215 |
| TFORF1255-TP53 | 215 |
| TFORF1590-ZNF773 | 215 |
| TFORF1643-ZNF426 | 215 |
| TFORF1846-ETS1 | 215 |
| TFORF1915-RUNX2 | 215 |
| TFORF2254-HMGN3 | 215 |
| TFORF2288-HES4 | 215 |
| TFORF2974-NR1H2 | 215 |
| TFORF0291-MEF2C | 216 |
| TFORF0332-ZNF333 | 216 |
| TFORF0718-TEF | 216 |
| TFORF0949-EMX1 | 216 |
| TFORF1407-ZNF559 | 216 |
| TFORF1454-TFEC | 216 |
| TFORF1551-TAZ | 216 |
| TFORF1997-ZNF248 | 216 |
| TFORF2246-TCF12 | 216 |
| TFORF3321-PA2G4 | 216 |
| TFORF0428-PBX1 | 217 |
| TFORF0648-NR113 | 217 |
| TFORF0756-JUND | 217 |
| TFORF0800-ATF3 | 217 |
| TFORF0883-HOXB7 | 217 |
| TFORF2480-MXD4 | 217 |
| TFORF0359-TUB | 218 |
| TFORF2947-HOXC8 | 218 |
| TFORF0057-MAF | 219 |
| TFORF0696-GTF3A | 219 |
| TFORF2757-BARHL1 | 219 |
| TFORF3337-HDAC1 | 219 |
| TFORF0781-ZNF93 | 220 |
| TFORF1562-ZNF688 | 220 |
| TFORF1934-MEIS2 | 220 |
| TFORF2446-ZIK1 | 220 |
| TFORF0152-THAP6 | 221 |
| TFORF0436-ZSCAN31 | 221 |
| TFORF0270-TCF7 | 222 |
| TFORF0894-ING4 | 222 |
| TFORF1485-NANOG | 222 |
| TFORF2474-ONECUT3 | 222 |
| TFORF3146-HSF1 | 222 |
| TFORF1393-PMS1 | 223 |
| TFORF3193-DND1 | 223 |
| TFORF3349-ERCC8 | 223 |
| TFORF0367-NFYC | 224 |
| TFORF2111-NR4A1 | 224 |
| TFORF2349-HOXD8 | 224 |
| TFORF0206-ETV3L | 225 |
| TFORF0525-PAX4 | 225 |
| TFORF1425-ZNF211 | 225 |
| TFORF2599-RORA | 225 |
| TFORF2834-IKZF3 | 225 |
| TFORF0017-IRX5 | 226 |
| TFORF0109-SMAD6 | 226 |
| TFORF0570-ZNF502 | 226 |
| TFORF0879-HOXB3 | 226 |
| TFORF1160-EOMES | 226 |
| TFORF3136-HEY1 | 226 |
| TFORF2205-POU2F3 | 227 |
| TFORF2905-DACH2 | 227 |
| TFORF2988-OTX2 | 227 |
| TFORF0513-PAX5 | 228 |
| TFORF0734-ZNF488 | 228 |
| TFORF1984-ZNF534 | 228 |
| TFORF3078-XRCC4 | 228 |
| TFORF3511-SOX14 | 228 |
| TFORF0119-ELMSAN1 | 229 |
| TFORF2339-ZNF19 | 229 |
| TFORF3010-NR113 | 229 |
| TFORF3371-MYBL1 | 229 |
| TFORF0124-OLIG1 | 230 |
| TFORF0437-ZSCAN31 | 230 |
| TFORF0893-ING4 | 230 |
| TFORF1076-NFATC4 | 230 |
| TFORF1311-RBMS1 | 230 |
| TFORF2875-ELF3 | 230 |
| TFORF3167-ZNF512B | 230 |
| TFORF0976-GFI1B | 231 |
| TFORF1879-CREBZF | 231 |
| TFORF1053-HKR1 | 232 |
| TFORF1266-ZNF135 | 232 |
| TFORF1294-ZFP64 | 232 |
| TFORF0023-IRX2 | 233 |
| TFORF0053-MAZ | 233 |
| TFORF0408-CTCFL | 233 |
| TFORF0472-ZNF221 | 233 |
| TFORF3280-DDB2 | 233 |
| TFORF0058-MAF | 234 |
| TFORF0878-HOXB2 | 234 |
| TFORF2166-GRHL3 | 234 |
| TFORF2975-NAT10 | 234 |
| TFORF3025-MYF6 | 234 |
| TFORF3108-ASCL2 | 234 |
| TFORF3129-SMAD7 | 234 |
| TFORF3362-MYOG | 234 |
| TFORF3541-THAP6 | 234 |
| TFORF1343-FEZF1 | 235 |
| TFORF3220-ZNF449 | 235 |
| TFORF3509-GATAD2B | 235 |
| TFORF0536-PAX2 | 236 |
| TFORF1034-WT1 | 236 |
| TFORF2167-GRHL3 | 236 |
| TFORF2306-ETV1 | 236 |
| TFORF2347-HOXD8 | 236 |
| TFORF2702-ESRRG | 236 |
| TFORF3272-PRDM4 | 236 |
| TFORF0862-NR1H4 | 237 |
| TFORF0941-LMO1 | 237 |
| TFORF0955-CDIP1 | 237 |
| TFORF1140-HMBOX1 | 237 |
| TFORF1146-HOXA4 | 237 |
| TFORF2726-ZFP1 | 237 |
| TFORF2806-ETV7 | 237 |
| TFORF2862-SREBF1 | 237 |
| TFORF3518-NHLH2 | 237 |
| TFORF0401-ZNF2 | 238 |
| TFORF0406-CTCFL | 238 |
| TFORF0534-ZNF584 | 238 |
| TFORF0797-ATF1 | 238 |
| TFORF1919-RUNX1 | 238 |
| TFORF2164-GRHL2 | 238 |
| TFORF0295-MEF2A | 239 |
| TFORF0445-ZNF805 | 239 |
| TFORF0789-DBX2 | 239 |
| TFORF0991-KLF8 | 239 |
| TFORF1152-MYOCD | 239 |
| TFORF1179-ZNF799 | 239 |
| TFORF1348-RPA2 | 239 |
| TFORF1854-ZBTB5 | 239 |
| TFORF1943-MIER1 | 239 |
| TFORF1954-ESR2 | 239 |
| TFORF1983-TBX5 | 239 |
| TFORF2848-TCF21 | 239 |
| TFORF3300-MEIS2 | 239 |
| TFORF3313-ZBTB25 | 239 |
| TFORF3361-MSC | 239 |
| TFORF0258-TCF4 | 240 |
| TFORF2213-CREB3L1 | 240 |
| TFORF2748-NKX6-3 | 240 |
| TFORF3443-SP4 | 240 |
| TFORF1078-NFATC4 | 241 |
| TFORF2275-SLC22A1 | 241 |
| TFORF2533-SOX1 | 241 |
| TFORF2934-TFEB | 241 |
| TFORF3178-TFDP2 | 241 |
| TFORF3388-YEATS4 | 241 |
| TFORF0038-SP7 | 242 |
| TFORF0249-CAMTA2 | 242 |
| TFORF0256-TCF4 | 242 |
| TFORF0342-ETF1 | 242 |
| TFORF0496-EGR2 | 242 |
| TFORF0526-PAX7 | 242 |
| TFORF0972-ZNF124 | 242 |
| TFORF1466-HOXD12 | 242 |
| TFORF1871-POU2F1 | 242 |
| TFORF2146-ZNF529 | 242 |
| TFORF2691-TEAD2 | 242 |
| TFORF3346-NHP2 | 242 |
| TFORF3376-TOX | 242 |
| TFORF3382-ZNF140 | 242 |
| TFORF3431-NR3C2 | 242 |
| TFORF0452-THRA | 243 |
| TFORF1581-ATMIN | 243 |
| TFORF2105-ZBTB34 | 243 |
| TFORF2153-BCL11A | 243 |
| TFORF2184-ZNF415 | 243 |
| TFORF3360-HOXB13 | 243 |
| TFORF0022-IRX3 | 244 |
| TFORF0110-SMAD7 | 244 |
| TFORF0121-BARX2 | 244 |
| TFORF0149-THAP4 | 244 |
| TFORF0617-ZSCAN26 | 244 |
| TFORF0921-BSX | 244 |
| TFORF1342-POU5F2 | 244 |
| TFORF1542-ZNF286B | 244 |
| TFORF1552-SP110 | 244 |
| TFORF1625-ZNF517 | 244 |
| TFORF1819-ZNF138 | 244 |
| TFORF2173-ZNF419 | 244 |
| TFORF2556-ADNP2 | 244 |
| TFORF2645-PAX3 | 244 |
| TFORF3041-SOX15 | 244 |
| TFORF3132-ESR2 | 244 |
| TFORF3237-SOX10 | 244 |
| TFORF3484-ZNF398 | 244 |
| TFORF0937-SS18 | 245 |
| TFORF1586-ZNF772 | 245 |
| TFORF1801-TBX10 | 245 |
| TFORF2925-ZFP57 | 245 |
| TFORF0360-TUB | 246 |
| TFORF0728-MLXIP | 246 |
| TFORF1670-TP73 | 246 |
| TFORF2465-IRF6 | 246 |
| TFORF2647-PAX3 | 246 |
| TFORF3027-SP6 | 246 |
| TFORF0288-MEF2C | 247 |
| TFORF0775-ZNF92 | 247 |
| TFORF0814-LHX1 | 247 |
| TFORF0815-LHX2 | 247 |
| TFORF0816-LHX3 | 247 |
| TFORF0818-LHX3 | 247 |
| TFORF0819-LHX5 | 247 |
| TFORF1459-LMX1A | 247 |
| TFORF1460-LMX1B | 247 |
| TFORF1461-LMX1B | 247 |
| TFORF1462-LMX1B | 247 |
| TFORF1471-TOX2 | 247 |
| TFORF1897-NR2C1 | 247 |
| TFORF2367-MBD4 | 247 |
| TFORF2386-MBD2 | 247 |
| TFORF3179-LHX9 | 247 |
| TFORF3212-ZKSCAN1 | 247 |
| TFORF0820-LHX6 | 248 |
| TFORF0821-LHX6 | 248 |
| TFORF0822-LHX6 | 248 |
| TFORF0824-LHX8 | 248 |
| TFORF0825-LHX8 | 248 |
| TFORF1825-ARNT | 248 |
| TFORF2625-CRTC3 | 248 |
| TFORF2873-ELF1 | 248 |
| TFORF0296-MEF2A | 249 |
| TFORF2058-NKX2-2 | 249 |
| TFORF3233-JUNB | 249 |
| TFORF0674-ZNF829 | 250 |
| TFORF2381-MBD1 | 250 |
| TFORF2530-SOX8 | 250 |
| TFORF2671-NEUROD4 | 250 |
| TFORF1517-FOXD4L4 | 251 |
| TFORF2375-MBD1 | 251 |
| TFORF2945-NEUROD1 | 251 |
| TFORF1012-ZNF546 | 252 |
| TFORF1641-PSIP1 | 252 |
| TFORF3260-ZNF524 | 252 |
| TFORF0010-ZNF879 | 253 |
| TFORF0391-FERD3L | 253 |
| TFORF0639-HOXC6 | 253 |
| TFORF1223-ZNF483 | 253 |
| TFORF2322-ZNF736 | 253 |
| TFORF2355-PROP1 | 253 |
| TFORF3079-ZNF846 | 253 |
| TFORF0577-GTF2A1L | 254 |
| TFORF0616-ZSCAN26 | 254 |
| TFORF1150-CDK7 | 254 |
| TFORF1502-GABPB1 | 254 |
| TFORF2684-ZMYND11 | 254 |
| TFORF2989-SLC45A2 | 254 |
| TFORF0164-SIX4 | 255 |
| TFORF0686-PRDM10 | 255 |
| TFORF1659-LEF1 | 255 |
| TFORF1799-USF2 | 255 |
| TFORF2376-MBD1 | 255 |
| TFORF2499-ASCL5 | 255 |
| TFORF3106-CCNH | 255 |
| TFORF0172-ONECUT2 | 256 |
| TFORF0745-PIAS2 | 256 |
| TFORF1334-POU3F3 | 256 |
| TFORF3100-ZNF212 | 256 |
| TFORF0895-ING4 | 257 |
| TFORF1483-TP63 | 257 |
| TFORF1147-HOXA3 | 258 |
| TFORF1638-BNIP3 | 258 |
| TFORF2332-ZNF492 | 258 |
| TFORF2884-FIGLA | 258 |
| TFORF2276-SLC22A1 | 259 |
| TFORF3083-FOSL2 | 259 |
| TFORF3548-THAP10 | 259 |
| TFORF0564-ZNF619 | 260 |
| TFORF1216-NHLH1 | 260 |
| TFORF1748-ZNF763 | 260 |
| TFORF1870-POU2F1 | 260 |
| TFORF1994-ZHX1 | 260 |
| TFORF2093-ZFP14 | 260 |
| TFORF2479-MESP2 | 260 |
| TFORF0035-SP3 | 261 |
| TFORF0338-MECOM | 261 |
| TFORF1058-NFATC1 | 261 |
| TFORF1124-E2F7 | 261 |
| TFORF2764-ZKSCAN5 | 261 |
| TFORF3401-GTF2B | 261 |
| TFORF0881-HOXB3 | 262 |
| TFORF3402-HSFY2 | 262 |
| TFORF0130-HMGB3 | 263 |
| TFORF1695-ZNF114 | 263 |
| TFORF2659-STAT2 | 263 |
| TFORF3022-RXRG | 263 |
| TFORF3073-LMO2 | 263 |
| TFORF0770-MSX2 | 264 |
| TFORF1167-EBF3 | 264 |
| TFORF1168-EBF2 | 264 |
| TFORF2224-YAP1 | 264 |
| TFORF3058-EBF1 | 264 |
| TFORF0104-SMAD9 | 265 |
| TFORF3195-ONECUT1 | 265 |
| TFORF0521-PAX5 | 266 |
| TFORF3074-LMO2 | 266 |
| TFORF0576-HOXA10 | 267 |
| TFORF1166-EBF4 | 267 |
| TFORF1522-NR2E3 | 267 |
| TFORF3475-IL18 | 267 |
| TFORF0088-ZNF408 | 268 |
| TFORF0123-OLIG2 | 268 |
| TFORF1433-NR3C1 | 268 |
| TFORF2938-SOX5 | 268 |
| TFORF0869-NR1H3 | 269 |
| TFORF0891-ING4 | 269 |
| TFORF1956-ESR2 | 269 |
| TFORF1718-LDB1 | 270 |
| TFORF2583-MIXL1 | 270 |
| TFORF3546-TSC22D3 | 270 |
| TFORF2218-YAP1 | 271 |
| TFORF2795-ZNF283 | 271 |
| TFORF2839-IKZF3 | 271 |
| TFORF3127-NFIL3 | 271 |
| TFORF0979-KLF7 | 272 |
| TFORF1328-TFAP2A | 272 |
| TFORF1331-TFAP2B | 272 |
| TFORF1332-TFAP2E | 272 |
| TFORF1635-HOXC11 | 272 |
| TFORF1644-ZNF425 | 272 |
| TFORF1939-MEIS1 | 272 |
| TFORF2064-NKX2-5 | 272 |
| TFORF2193-TFAM | 272 |
| TFORF2932-TFAP2A | 272 |
| TFORF0716-ZNF621 | 273 |
| TFORF2161-GRHL1 | 273 |
| TFORF2165-GRHL3 | 273 |
| TFORF2168-GRHL3 | 273 |
| TFORF2266-ANHX | 273 |
| TFORF2700-ESRRG | 273 |
| TFORF2755-ZNF440 | 273 |
| TFORF0106-TRERF1 | 274 |
| TFORF0544-ZFAT | 274 |
| TFORF0725-NFKB2 | 274 |
| TFORF1971-YY2 | 274 |
| TFORF3477-POU5F1 | 274 |
| TFORF0008-ZNF709 | 275 |
| TFORF0009-ZNF708 | 275 |
| TFORF0030-ZNF43 | 275 |
| TFORF0259-TCF4 | 275 |
| TFORF1301-HMG20A | 275 |
| TFORF1424-ZNF211 | 275 |
| TFORF1490-NR6A1 | 275 |
| TFORF1499-RBL1 | 275 |
| TFORF1500-RBL2 | 275 |
| TFORF1521-NR2E3 | 275 |
| TFORF1580-ZNF778 | 275 |
| TFORF1622-ZNF513 | 275 |
| TFORF1789-ZNF232 | 275 |
| TFORF2055-CDX1 | 275 |
| TFORF2127-ZNF746 | 275 |
| TFORF2738-PLK4 | 275 |
| TFORF2811-ETV7 | 275 |
| TFORF2823-IKZF1 | 275 |
| TFORF2838-IKZF3 | 275 |
| TFORF2895-MTA1 | 275 |
| TFORF2907-DACH2 | 275 |
| TFORF2963-NR2C2 | 275 |
| TFORF3008-CDX2 | 275 |
| TFORF3291-CDK9 | 275 |
| TFORF3320-HDX | 275 |
| TFORF3400-GMEB1 | 275 |
| TFORF3462-BRPF1 | 275 |
| TFORF0247-CAMTA2 | 276 |
| TFORF0440-ZFY | 276 |
| TFORF0456-HOXD9 | 276 |
| TFORF1060-NFATC1 | 276 |
| TFORF3427-NFIB | 276 |
| TFORF0465-NKX1-1 | 277 |
| TFORF1604-HINFP | 277 |
| TFORF1785-ZNF235 | 277 |
| TFORF3093-ZNF574 | 277 |
| TFORF3290-CTCF | 277 |
| TFORF3547-CBFB | 277 |
| TFORF0924-NR2F2 | 278 |
| TFORF1426-FOXK2 | 278 |
| TFORF1468-HOXD11 | 278 |
| TFORF1475-LIN54 | 278 |
| TFORF1505-ZNF696 | 278 |
| TFORF2200-POU4F1 | 278 |
| TFORF2274-SLC22A1 | 278 |
| TFORF3056-THRB | 278 |
| TFORF3493-ZNF791 | 278 |
| TFORF0405-CTCFL | 279 |
| TFORF1758-ZNF189 | 279 |
| TFORF1864-POU2F2 | 279 |
| TFORF1916-RUNX2 | 279 |
| TFORF2149-ZNF250 | 279 |
| TFORF2541-SOX5 | 279 |
| TFORF3210-VSX2 | 279 |
| TFORF3236-E2F8 | 279 |
| TFORF0318-ZNF117 | 280 |
| TFORF2282-MYBBP1A | 280 |
| TFORF2708-ARX | 280 |
| TFORF2774-KDM5C | 280 |
| TFORF3166-ZNF512B | 280 |
| TFORF3256-ZNF189 | 280 |
| TFORF3340-HDAC3 | 280 |
| TFORF0002-HIF3A | 281 |
| TFORF0190-RFX4 | 281 |
| TFORF0497-EGR3 | 281 |
| TFORF0684-PRDM13 | 281 |
| TFORF0866-GTF2I | 281 |
| TFORF0896-ZNF383 | 281 |
| TFORF1006-ZNF564 | 281 |
| TFORF1062-NFATC1 | 281 |
| TFORF1572-ZNF687 | 281 |
| TFORF1737-ZBTB7B | 281 |
| TFORF1768-ZNF28 | 281 |
| TFORF2026-NPAS3 | 281 |
| TFORF2117-TBPL2 | 281 |
| TFORF2364-CREB1 | 281 |
| TFORF2471-HDAC5 | 281 |
| TFORF3219-ZNF677 | 281 |
| TFORF0317-MEF2D | 282 |
| TFORF0498-EGR3 | 282 |
| TFORF1023-FOXI1 | 282 |
| TFORF1399-ZNF568 | 282 |
| TFORF1636-HOXC13 | 282 |
| TFORF1848-ETS1 | 282 |
| TFORF2043-ARID3A | 282 |
| TFORF2526-ERCC2 | 282 |
| TFORF2540-SOX5 | 282 |
| TFORF2581-MEIS3 | 282 |
| TFORF2653-ARNTL2 | 282 |
| TFORF2750-NKX6-1 | 282 |
| TFORF2965-NR2C2 | 282 |
| TFORF3046-ZNF418 | 282 |
| TFORF3112-RFXANK | 282 |
| TFORF3171-PSMB4 | 282 |
| TFORF3177-NR1D2 | 282 |
| TFORF3194-TLX3 | 282 |
| TFORF3200-RUNX3 | 282 |
| TFORF3255-ZNF175 | 282 |
| TFORF3404-ZNF511 | 282 |
| TFORF0193-RFX5 | 283 |
| TFORF1524-NFRKB | 283 |
| TFORF1704-E2F3 | 283 |
| TFORF2071-MYB | 283 |
| TFORF2074-MYB | 283 |
| TFORF1686-HNF1B | 284 |
| TFORF2456-IRF3 | 284 |
| TFORF0355-ZNF33A | 285 |
| TFORF0575-HOXA11 | 285 |
| TFORF2491-ZBTB17 | 285 |
| TFORF2902-DACH1 | 285 |
| TFORF2996-TGIF1 | 285 |
| TFORF3467-SUPT4H1 | 285 |
| TFORF3533-CDIP1 | 285 |
| TFORF0108-TRERF1 | 286 |
| TFORF1036-WT1 | 286 |
| TFORF2151-FOXG1 | 286 |
| TFORF2490-ZBTB17 | 286 |
| TFORF2666-ZBED3 | 286 |
| TFORF0900-ZNF382 | 287 |
| TFORF0973-ZNF124 | 287 |
| TFORF1513-NKX3-1 | 287 |
| TFORF2607-DMTF1 | 287 |
| TFORF2729-ZNF668 | 287 |
| TFORF0458-TRIM24 | 288 |
| TFORF1265-ZNF135 | 288 |
| TFORF1538-ZBTB46 | 288 |
| TFORF2553-AR | 288 |
| TFORF3538-HOXD10 | 288 |
| TFORF0431-AIRE | 289 |
| TFORF0561-ZNF616 | 289 |
| TFORF0922-NR2F1 | 289 |
| TFORF1319-KLF13 | 289 |
| TFORF1337-ZBTB7A | 289 |
| TFORF1964-CUX1 | 289 |
| TFORF2289-HES5 | 289 |
| TFORF2380-MBD1 | 289 |
| TFORF0842-ZSCAN10 | 290 |
| TFORF2527-ERCC3 | 290 |
| TFORF0598-SMARCB1 | 291 |
| TFORF0885-PPARG | 291 |
| TFORF1174-NRL | 291 |
| TFORF1868-MMP3 | 291 |
| TFORF2097-ZKSCAN1 | 291 |
| TFORF3491-NR113 | 291 |
| TFORF0039-SP7 | 292 |
| TFORF0459-CREB5 | 292 |
| TFORF1394-ZNF569 | 292 |
| TFORF1942-MIER1 | 292 |
| TFORF2056-NKX2-8 | 292 |
| TFORF3054-SALL4 | 292 |
| TFORF3317-ZNF75A | 292 |
| TFORF0024-FOXQ1 | 293 |
| TFORF0136-ZNF780B | 293 |
| TFORF0173-RELB | 293 |
| TFORF0232-ZNF599 | 293 |
| TFORF0489-ZFP91 | 293 |
| TFORF0568-ZNF618 | 293 |
| TFORF0590-FOXO6 | 293 |
| TFORF1189-ZNF571 | 293 |
| TFORF1338-ZBTB7C | 293 |
| TFORF1420-ZNF211 | 293 |
| TFORF2103-ZBTB32 | 293 |
| TFORF2549-SKI | 293 |
| TFORF0166-SIX6 | 294 |
| TFORF0649-NR113 | 294 |
| TFORF1435-NFKBIE | 294 |
| TFORF2457-IRF3 | 294 |
| TFORF3398-HLF | 294 |
| TFORF0251-VEZF1 | 295 |
| TFORF0320-MSLN | 295 |
| TFORF1261-TP53 | 295 |
| TFORF1492-FOSL1 | 295 |
| TFORF1615-GSX1 | 295 |
| TFORF2062-NKX2-6 | 295 |
| TFORF2068-FLII | 295 |
| TFORF2104-ZBTB34 | 295 |
| TFORF2169-BLZF1 | 295 |
| TFORF2728-ZFP1 | 295 |
| TFORF3104-NKX2-5 | 295 |
| TFORF3242-ZNF136 | 295 |
| TFORF3497-ZNF280C | 295 |
| TFORF0186-DMRTC1 | 296 |
| TFORF1199-ZNF579 | 296 |
| TFORF1637-HOXC12 | 296 |
| TFORF2154-BCL11A | 296 |
| TFORF2290-HES6 | 296 |
| TFORF2780-ZNF30 | 296 |
| TFORF2982-ZNF420 | 296 |
| TFORF3011-ZNF10 | 296 |
| TFORF3539-ZNF747 | 296 |
| TFORF0864-GTF2I | 297 |
| TFORF0956-MYF5 | 297 |
| TFORF1114-RCOR3 | 297 |
| TFORF1234-AFF2 | 297 |
| TFORF3323-ZNF626 | 297 |
| TFORF0421-SKIL | 298 |
| TFORF0637-HOXC4 | 298 |
| TFORF1508-MTF1 | 298 |
| TFORF1708-PPP1R13L | 298 |
| TFORF1803-USF1 | 298 |
| TFORF2159-BCL11B | 298 |
| TFORF2222-YAP1 | 298 |
| TFORF2396-ZNF396 | 298 |
| TFORF2559-TLX1 | 298 |
| TFORF3116-HEYL | 298 |
| TFORF0790-DBX1 | 299 |
| TFORF1464-VAV1 | 299 |
| TFORF2066-FLII | 299 |
| TFORF2245-TCF12 | 299 |
| TFORF2762-ZKSCAN3 | 299 |
| TFORF2883-BHLHA9 | 299 |
| TFORF3018-NEUROG3 | 299 |
| TFORF3456-ZNF426 | 299 |
| TFORF0112-SMAD2 | 300 |
| TFORF0943-LMO2 | 300 |
| TFORF1180-ZNF799 | 300 |
| TFORF1300-MAEL | 300 |
| TFORF1602-ZNF256 | 300 |
| TFORF1903-TCF7L2 | 300 |
| TFORF2472-HDAC5 | 300 |
| TFORF2914-NFIB | 300 |
| TFORF3397-ZIK1 | 300 |
| TFORF3501-CERS6 | 300 |
| TFORF0174-RELA | 301 |
| TFORF0409-CTCFL | 301 |
| TFORF0813-CDCA7L | 301 |
| TFORF1139-LRRFIP1 | 301 |
| TFORF1239-ZNF431 | 301 |
| TFORF1310-TTF1 | 301 |
| TFORF1624-ZNF516 | 301 |
| TFORF1894-AHRR | 301 |
| TFORF1896-NR2C1 | 301 |
| TFORF1932-JUN | 301 |
| TFORF2022-NPAS4 | 301 |
| TFORF2754-ZNF446 | 301 |
| TFORF3249-ZNF181 | 301 |
| TFORF0098-MSGN1 | 302 |
| TFORF0163-PES1 | 302 |
| TFORF0794-ATF7 | 302 |
| TFORF0970-ZNF124 | 302 |
| TFORF1245-PKNOX2 | 302 |
| TFORF2089-ZNF442 | 302 |
| TFORF2092-ZBTB2 | 302 |
| TFORF2284-HES2 | 302 |
| TFORF2318-ZNF732 | 302 |
| TFORF2412-ZNF705D | 302 |
| TFORF2478-MESP1 | 302 |
| TFORF2531-SOX9 | 302 |
| TFORF3119-DDIT3 | 302 |
| TFORF3224-HMGB1 | 302 |
| TFORF3252-ZNF77 | 302 |
| TFORF3399-POU2F2 | 302 |
| TFORF0998-ZNF302 | 303 |
| TFORF1191-ZNF573 | 303 |
| TFORF1724-ZNF691 | 303 |
| TFORF1917-RUNX2 | 303 |
| TFORF1973-TBX3 | 303 |
| TFORF2078-FLI1 | 303 |
| TFORF2766-ZKSCAN7 | 303 |
| TFORF0269-TCF4 | 304 |
| TFORF0768-ZNF551 | 304 |
| TFORF1612-TADA2A | 304 |
| TFORF1757-ZNF189 | 304 |
| TFORF2913-NFIB | 304 |
| TFORF0005-HIF3A | 305 |
| TFORF0646-NR113 | 305 |
| TFORF0961-ERG | 305 |
| TFORF1760-ZNF25 | 305 |
| TFORF2730-ZNF668 | 305 |
| TFORF3113-RFXANK | 305 |
| TFORF3231-ZNF423 | 305 |
| TFORF3503-CDK7 | 305 |
| TFORF0601-ZNF726 | 306 |
| TFORF2235-ZNF880 | 306 |
| TFORF3302-SPIC | 306 |
| TFORF0045-ZNF671 | 307 |
| TFORF1869-POU2F1 | 307 |
| TFORF2788-OTX2 | 307 |
| TFORF2801-ETV2 | 307 |
| TFORF3110-TGIF2LY | 307 |
| TFORF1158-EOMES | 308 |
| TFORF1714-LDB2 | 308 |
| TFORF3033-NFKBIA | 308 |
| TFORF3476-SNAI1 | 308 |
| TFORF0486-ZFP90 | 309 |
| TFORF0640-NHP2 | 309 |
| TFORF0892-ING4 | 309 |
| TFORF1009-ZNF544 | 309 |
| TFORF1128-THAP3 | 309 |
| TFORF1363-ELOF1 | 309 |
| TFORF1409-ZNF559 | 309 |
| TFORF1515-NANOGNB | 309 |
| TFORF1947-MIER1 | 309 |
| TFORF2725-ZNF74 | 309 |
| TFORF2803-ETV2 | 309 |
| TFORF3512-SOX14 | 309 |
| TFORF0015-DRAP1 | 310 |
| TFORF2311-RXRG | 310 |
| TFORF2633-TFDP2 | 310 |
| TFORF2705-ESRRB | 310 |
| TFORF0007-TULP4 | 311 |
| TFORF1181-TBX21 | 311 |
| TFORF1372-ZNF143 | 311 |
| TFORF1603-HINFP | 311 |
| TFORF1940-JUP | 311 |
| TFORF3425-ZNF559 | 311 |
| TFORF0135-ZNF780A | 312 |
| TFORF0812-CDCA7L | 312 |
| TFORF0860-NR1H4 | 312 |
| TFORF1159-EOMES | 312 |
| TFORF1376-ZNF146 | 312 |
| TFORF1473-LIN54 | 312 |
| TFORF2099-SOHLH2 | 312 |
| TFORF2370-MBD4 | 312 |
| TFORF2405-NFE2L1 | 312 |
| TFORF2515-PDCD2 | 312 |
| TFORF2741-ZNF484 | 312 |
| TFORF2885-GLI2 | 312 |
| TFORF3007-SMAD1 | 312 |
| TFORF3379-ACTL6A | 312 |
| TFORF0591-FOXO4 | 313 |
| TFORF0999-ZNF302 | 313 |
| TFORF1054-PLAGL1 | 313 |
| TFORF1613-TADA2A | 313 |
| TFORF1759-ALYREF | 313 |
| TFORF1816-SATB1 | 313 |
| TFORF2303-CIITA | 313 |
| TFORF2784-GLIS3 | 313 |
| TFORF3029-ATOH1 | 313 |
| TFORF3087-MAFF | 313 |
| TFORF3267-SNAPC2 | 313 |
| TFORF3283-HOXA5 | 313 |
| TFORF3374-VAX2 | 313 |
| TFORF3521-THAP8 | 313 |
| TFORF0182-SHOX2 | 314 |
| TFORF1141-HMBOX1 | 314 |
| TFORF1322-KLF10 | 314 |
| TFORF2069-MYC | 314 |
| TFORF2150-ZNF250 | 314 |
| TFORF0032-GSC2 | 315 |
| TFORF1182-TBX20 | 315 |
| TFORF2287-HES4 | 315 |
| TFORF3251-PFDN5 | 315 |
| TFORF0230-ZNF595 | 316 |
| TFORF0654-NR113 | 316 |
| TFORF0854-VSX1 | 316 |
| TFORF1481-TP63 | 316 |
| TFORF2382-MBD1 | 316 |
| TFORF2590-CERS3 | 316 |
| TFORF2859-ID1 | 316 |
| TFORF2870-HMX1 | 316 |
| TFORF1086-GTF2E1 | 317 |
| TFORF1330-TFAP2C | 317 |
| TFORF1699-E2F5 | 317 |
| TFORF2180-ZNF417 | 317 |
| TFORF2715-ZNF713 | 317 |
| TFORF2783-GLIS3 | 317 |
| TFORF2785-GLIS1 | 317 |
| TFORF2786-GLIS1 | 317 |
| TFORF2941-DLX5 | 317 |
| TFORF3275-HMGA1 | 317 |
| TFORF3417-TCF7L2 | 317 |
| TFORF0467-CARHSP1 | 318 |
| TFORF1094-HSFX2 | 318 |
| TFORF1487-NR6A1 | 318 |
| TFORF1640-PSIP1 | 318 |
| TFORF1863-POU2F2 | 318 |
| TFORF2605-MYRF | 318 |
| TFORF3043-ZNF75D | 318 |
| TFORF3152-ZFP3 | 318 |
| TFORF3253-RBPJ | 318 |
| TFORF3333-PUF60 | 318 |
| TFORF0581-ZNF101 | 319 |
| TFORF0671-ZNF821 | 319 |
| TFORF0701-PAXBP1 | 319 |
| TFORF1142-HMBOX1 | 319 |
| TFORF1309-TTF1 | 319 |
| TFORF1676-RUNX1T1 | 319 |
| TFORF1775-ZNF500 | 319 |
| TFORF1829-DMRTA2 | 319 |
| TFORF1937-MEIS2 | 319 |
| TFORF2174-ZNF419 | 319 |
| TFORF2241-GATA3 | 319 |
| TFORF2371-ZNF468 | 319 |
| TFORF3133-TCF7 | 319 |
| TFORF0852-ZIC4 | 320 |
| TFORF1171-MLX | 320 |
| TFORF1697-E2F6 | 320 |
| TFORF2810-ETV7 | 320 |
| TFORF2852-HMGA2 | 320 |
| TFORF3531-HMGN3 | 320 |
| TFORF0308-CREM | 321 |
| TFORF0380-SMARCA2 | 321 |
| TFORF0925-NR2F2 | 321 |
| TFORF1252-TP53 | 321 |
| TFORF1506-HEY2 | 321 |
| TFORF1588-ZNF773 | 321 |
| TFORF2511-PDCD2 | 321 |
| TFORF2655-STAT6 | 321 |
| TFORF1143-HMBOX1 | 322 |
| TFORF1312-AEBP2 | 322 |
| TFORF1716-LDB2 | 322 |
| TFORF3159-SMAD2 | 322 |
| TFORF3312-ELK4 | 322 |
| TFORF1063-NFATC1 | 323 |
| TFORF2079-FLI1 | 323 |
| TFORF2582-NR1D1 | 323 |
| TFORF2828-IKZF3 | 323 |
| TFORF0289-MEF2C | 324 |
| TFORF0438-ZSCAN30 | 324 |
| TFORF0571-MEOX1 | 324 |
| TFORF0750-ZNF131 | 324 |
| TFORF0809-SOHLH1 | 324 |
| TFORF2125-ZNF747 | 324 |
| TFORF2356-ZNF648 | 324 |
| TFORF2387-MBD2 | 324 |
| TFORF2686-ZMYND11 | 324 |
| TFORF3351-ARNTL | 324 |
| TFORF0275-TCF3 | 325 |
| TFORF0795-ATF7 | 325 |
| TFORF1845-ALX4 | 325 |
| TFORF2779-BATF | 325 |
| TFORF3305-ESR1 | 325 |
| TFORF0580-ZNF107 | 326 |
| TFORF1038-WT1 | 326 |
| TFORF1652-SPI1 | 326 |
| TFORF1675-RUNX1T1 | 326 |
| TFORF3101-PES1 | 326 |
| TFORF0608-MAFA | 327 |
| TFORF0721-ZNF727 | 327 |
| TFORF1155-RERE | 327 |
| TFORF2901-DACH1 | 327 |
| TFORF3157-SMARCD1 | 327 |
| TFORF3517-SOX12 | 327 |
| TFORF0457-TRIM24 | 328 |
| TFORF0806-SMARCC2 | 328 |
| TFORF1064-NFATC1 | 328 |
| TFORF1374-ZNF143 | 328 |
| TFORF1812-ZNF438 | 328 |
| TFORF1873-BBX | 328 |
| TFORF2003-FOXD3 | 328 |
| TFORF2139-ZNF527 | 328 |
| TFORF2598-RORA | 328 |
| TFORF2959-KAT2A | 328 |
| TFORF0224-ZNF592 | 329 |
| TFORF0443-ZFX | 329 |
| TFORF0803-ATF2 | 329 |
| TFORF0861-NR1H4 | 329 |
| TFORF0986-GZF1 | 329 |
| TFORF1095-EN1 | 329 |
| TFORF1148-HOXA2 | 329 |
| TFORF1321-KLF11 | 329 |
| TFORF1585-ZNF772 | 329 |
| TFORF1661-ARID5B | 329 |
| TFORF1906-TCF7L2 | 329 |
| TFORF1999-TAL1 | 329 |
| TFORF2031-PRDM1 | 329 |
| TFORF2272-PRDM5 | 329 |
| TFORF2316-ETV4 | 329 |
| TFORF2420-ZNF705B | 329 |
| TFORF2445-ZIK1 | 329 |
| TFORF2536-SOX6 | 329 |
| TFORF2542-SOX5 | 329 |
| TFORF2609-FOXA2 | 329 |
| TFORF2744-T | 329 |
| TFORF3037-IRF2 | 329 |
| TFORF0370-NFYC | 330 |
| TFORF0595-PLAG1 | 330 |
| TFORF0968-HAND1 | 330 |
| TFORF1067-NFATC2 | 330 |
| TFORF1250-ZNF133 | 330 |
| TFORF1484-TP63 | 330 |
| TFORF1907-TCF7L2 | 330 |
| TFORF2449-GBX1 | 330 |
| TFORF2487-ZBTB10 | 330 |
| TFORF2944-SATB2 | 330 |
| TFORF3090-ZNF260 | 330 |
| TFORF3115-GSC | 330 |
| TFORF3122-SATB1 | 330 |
| TFORF3140-ZNF718 | 330 |
| TFORF3188-ZNF394 | 330 |
| TFORF3202-ZNF816 | 330 |
| TFORF3223-ZMIZ2 | 330 |
| TFORF3263-ZNF26 | 330 |
| TFORF3269-EZH1 | 330 |
| TFORF3276-ZBTB14 | 330 |
| TFORF3342-TP53BP2 | 330 |
| TFORF3372-DAXX | 330 |
| TFORF3446-ZNF786 | 330 |
| TFORF0077-KCNIP4 | 331 |
| TFORF0233-MNX1 | 331 |
| TFORF0990-KLF8 | 331 |
| TFORF1403-ZNF566 | 331 |
| TFORF1740-ZNF768 | 331 |
| TFORF2612-ZNF276 | 331 |
| TFORF2797-TAF7 | 331 |
| TFORF3430-FXN | 331 |
| TFORF3469-ZNF438 | 331 |
| TFORF0357-ZNF33A | 332 |
| TFORF0500-PPARGC1A | 332 |
| TFORF0940-ZNF639 | 332 |
| TFORF1469-TOX3 | 332 |
| TFORF1685-HNF1B | 332 |
| TFORF1720-DMRT2 | 332 |
| TFORF1833-MTERF1 | 332 |
| TFORF1972-TBX2 | 332 |
| TFORF2259-SOX13 | 332 |
| TFORF3217-ZNF227 | 332 |
| TFORF3268-ZNF34 | 332 |
| TFORF0185-SHOX2 | 333 |
| TFORF0212-FOXP3 | 333 |
| TFORF0368-NFYC | 333 |
| TFORF1230-AFF3 | 333 |
| TFORF1390-PMS1 | 333 |
| TFORF1859-NR4A3 | 333 |
| TFORF2638-TFDP1 | 333 |
| TFORF3209-ZBTB9 | 333 |
| TFORF3453-PREB | 333 |
| TFORF3524-MAEL | 333 |
| TFORF0767-ZNF550 | 334 |
| TFORF1400-ZNF567 | 334 |
| TFORF1687-HNF1B | 334 |
| TFORF2858-ID3 | 334 |
| TFORF2926-BCL6B | 334 |
| TFORF2962-NR2C2 | 334 |
| TFORF3088-E4F1 | 334 |
| TFORF0197-RFX1 | 335 |
| TFORF0846-YBX1 | 335 |
| TFORF1116-RCOR1 | 335 |
| TFORF1694-E4F1 | 335 |
| TFORF1732-HDX | 335 |
| TFORF1796-WDHD1 | 335 |
| TFORF2163-GRHL2 | 335 |
| TFORF2979-NROB2 | 335 |
| TFORF3097-ZNF76 | 335 |
| TFORF3124-SMAD3 | 335 |
| TFORF3204-LITAF | 335 |
| TFORF3227-RPA2 | 335 |
| TFORF3286-NR2F6 | 335 |
| TFORF3516-RNF138 | 335 |
| TFORF0369-NFYC | 336 |
| TFORF0554-ZNF610 | 336 |
| TFORF0786-FOXN1 | 336 |
| TFORF0884-HOXB4 | 336 |
| TFORF0946-LMO3 | 336 |
| TFORF1340-POU5F1 | 336 |
| TFORF1405-ZNF566 | 336 |
| TFORF1569-ZNF680 | 336 |
| TFORF1579-ZNF778 | 336 |
| TFORF1617-NF1 | 336 |
| TFORF1647-ZNF358 | 336 |
| TFORF1766-ZNF23 | 336 |
| TFORF1793-CNBP | 336 |
| TFORF2302-CIITA | 336 |
| TFORF2441-FUS | 336 |
| TFORF2488-ZBTB10 | 336 |
| TFORF2538-SOX4 | 336 |
| TFORF2833-IKZF3 | 336 |
| TFORF2845-TCF24 | 336 |
| TFORF3098-ZNF467 | 336 |
| TFORF3197-STAT5A | 336 |
| TFORF0157-EWSR1 | 337 |
| TFORF0245-CAMTA2 | 337 |
| TFORF0412-CTCFL | 337 |
| TFORF0585-ZNF329 | 337 |
| TFORF0749-ZNF131 | 337 |
| TFORF0902-PTF1A | 337 |
| TFORF1007-ZNF544 | 337 |
| TFORF1011-ZNF546 | 337 |
| TFORF1132-BNC1 | 337 |
| TFORF1361-SP100 | 337 |
| TFORF1523-NFRKB | 337 |
| TFORF1601-ZNF195 | 337 |
| TFORF1648-ZNF429 | 337 |
| TFORF1674-RUNX1T1 | 337 |
| TFORF1755-ZNF182 | 337 |
| TFORF1949-MIER2 | 337 |
| TFORF1970-ZNF233 | 337 |
| TFORF2070-MYB | 337 |
| TFORF2209-CREB3L3 | 337 |
| TFORF2519-ZFP37 | 337 |
| TFORF2627-NONO | 337 |
| TFORF2644-PAX3 | 337 |
| TFORF2768-KDM5D | 337 |
| TFORF2880-ELF2 | 337 |
| TFORF2888-BRPF1 | 337 |
| TFORF3061-SNAPC3 | 337 |
| TFORF3081-ELK3 | 337 |
| TFORF3190-ZNF554 | 337 |
| TFORF3295-SPDEF | 337 |
| TFORF3369-BLZF1 | 337 |
| TFORF3478-FOXD4 | 337 |
| TFORF3507-NOCT | 337 |
| TFORF0175-RELA | 338 |
| TFORF1315-ZNF813 | 338 |
| TFORF2040-TP53BP2 | 338 |
| TFORF2175-ZNF419 | 338 |
| TFORF2936-FOS | 338 |
| TFORF2973-PPARD | 338 |
| TFORF0114-SMAD3 | 339 |
| TFORF0667-TGFB111 | 339 |
| TFORF1839-EZH2 | 339 |
| TFORF2133-MNAT1 | 339 |
| TFORF3139-PPARG | 339 |
| TFORF0352-FOXL1 | 340 |
| TFORF1077-NFATC4 | 340 |
| TFORF1098-UBP1 | 340 |
| TFORF1567-ZNF683 | 340 |
| TFORF2320-TAF1A | 340 |
| TFORF0793-ATF5 | 341 |
| TFORF1264-ZNF135 | 341 |
| TFORF2460-IRF7 | 341 |
| TFORF2461-IRF7 | 341 |
| TFORF0294-MEF2A | 342 |
| TFORF1598-ZNF195 | 342 |
| TFORF2027-NPAS2 | 342 |
| TFORF2751-ZNF444 | 342 |
| TFORF0980-KLF7 | 343 |
| TFORF2051-ATF6B | 343 |
| TFORF2898-MTA3 | 343 |
| TFORF0273-TCF3 | 344 |
| TFORF0482-DMRTB1 | 344 |
| TFORF1005-POU1F1 | 344 |
| TFORF1355-ILF3 | 344 |
| TFORF1815-ZNF433 | 344 |
| TFORF2723-ZNF76 | 344 |
| TFORF2840-IKZF3 | 344 |
| TFORF2951-TWIST2 | 344 |
| TFORF0101-FOXC2 | 345 |
| TFORF0105-TRERF1 | 345 |
| TFORF1048-PLAGL2 | 345 |
| TFORF1272-DUXA | 345 |
| TFORF1537-ZBTB45 | 345 |
| TFORF1931-XRCC6 | 345 |
| TFORF2094-DEK | 345 |
| TFORF2199-RXRA | 345 |
| TFORF2777-KDM5C | 345 |
| TFORF3448-TP63 | 345 |
| TFORF0856-VSX1 | 346 |
| TFORF1066-NFATC2 | 346 |
| TFORF1705-E2F2 | 346 |
| TFORF1926-ZBTB25 | 346 |
| TFORF2262-ZFP28 | 346 |
| TFORF3047-ZNF266 | 346 |
| TFORF0029-ZNF43 | 347 |
| TFORF0113-SMAD3 | 347 |
| TFORF0687-PRDM10 | 347 |
| TFORF0758-OVOL3 | 347 |
| TFORF1055-PLAGL1 | 347 |
| TFORF1090-TULP1 | 347 |
| TFORF1587-ZNF772 | 347 |
| TFORF1593-ZNF197 | 347 |
| TFORF1667-TP73 | 347 |
| TFORF1982-TBX5 | 347 |
| TFORF2119-HOPX | 347 |
| TFORF2182-ZNF415 | 347 |
| TFORF2410-ZNF705G | 347 |
| TFORF2787-DCP1A | 347 |
| TFORF3103-HOXA1 | 347 |
| TFORF3214-ZNF484 | 347 |
| TFORF1650-ZSCAN5A | 348 |
| TFORF2307-ETV1 | 348 |
| TFORF2458-IRF3 | 348 |
| TFORF2648-PAX3 | 348 |
| TFORF2761-ZKSCAN3 | 348 |
| TFORF2931-LHX4 | 348 |
| TFORF0287-MEF2C | 349 |
| TFORF0620-ZSCAN21 | 349 |
| TFORF0934-ZNF85 | 349 |
| TFORF1726-ZNF692 | 349 |
| TFORF1763-ZNF26 | 349 |
| TFORF1927-ZNF75D | 349 |
| TFORF2667-ZBED4 | 349 |
| TFORF2727-ZFP1 | 349 |
| TFORF0142-FXN | 350 |
| TFORF0262-TCF4 | 350 |
| TFORF0303-CREM | 350 |
| TFORF0550-PAX8 | 350 |
| TFORF0783-SEBOX | 350 |
| TFORF0828-ZNF585B | 350 |
| TFORF1059-NFATC1 | 350 |
| TFORF1417-ZNF214 | 350 |
| TFORF1642-ZNF426 | 350 |
| TFORF1656-SPIB | 350 |
| TFORF2128-ZNF740 | 350 |
| TFORF2610-FOXA2 | 350 |
| TFORF2721-ZNF79 | 350 |
| TFORF2983-SUZ12 | 350 |
| TFORF3149-ZNF254 | 350 |
| TFORF3297-HIF1A | 350 |
| TFORF0168-SIX2 | 351 |
| TFORF0427-PBX1 | 351 |
| TFORF1830-ZNF717 | 351 |
| TFORF2447-ZIK1 | 351 |
| TFORF2958-SP1 | 351 |
| TFORF0226-ZNF593 | 352 |
| TFORF0347-FOXB1 | 352 |
| TFORF0778-SHOX | 352 |
| TFORF1220-HELT | 352 |
| TFORF2348-HOXD8 | 352 |
| TFORF0572-MEOX1 | 353 |
| TFORF0573-MEOX2 | 353 |
| TFORF0715-ZNF620 | 353 |
| TFORF1008-ZNF544 | 353 |
| TFORF1969-ZNF233 | 353 |
| TFORF2484-CBX2 | 353 |
| TFORF2693-TEAD4 | 353 |
| TFORF2863-SREBF1 | 353 |
| TFORF3435-SUPT5H | 353 |
| TFORF3479-ZNF132 | 353 |
| TFORF0593-UNCX | 354 |
| TFORF0945-LMO3 | 354 |
| TFORF1429-NR3C2 | 354 |
| TFORF2300-HIC1 | 354 |
| TFORF2496-ASCL3 | 354 |
| TFORF2497-ASCL1 | 354 |
| TFORF2498-ASCL4 | 354 |
| TFORF2534-SOX6 | 354 |
| TFORF2539-SOX5 | 354 |
| TFORF2596-RORB | 354 |
| TFORF3062-ZNF549 | 354 |
| TFORF3250-ZKSCAN8 | 354 |
| TFORF3367-ZNF281 | 354 |
| TFORF3472-NFATC3 | 354 |
| TFORF0187-DMRTC2 | 355 |
| TFORF0395-ZNF7 | 355 |
| TFORF0833-GTF2F2 | 355 |
| TFORF1089-TULP1 | 355 |
| TFORF1203-SETDB2 | 355 |
| TFORF1679-RUNX1T1 | 355 |
| TFORF2293-HES7 | 355 |
| TFORF2451-MLLT10 | 355 |
| TFORF3229-MAX | 355 |
| TFORF0063-GTF3C2 | 356 |
| TFORF0072-CSDE1 | 356 |
| TFORF0122-BARX1 | 356 |
| TFORF0274-TCF3 | 356 |
| TFORF0378-SMARCA1 | 356 |
| TFORF0512-TSHZ1 | 356 |
| TFORF0859-ZIC3 | 356 |
| TFORF0926-ATOH7 | 356 |
| TFORF0947-EMX2 | 356 |
| TFORF0995-ZNF304 | 356 |
| TFORF1073-NFATC4 | 356 |
| TFORF1306-ZSCAN9 | 356 |
| TFORF1308-JARID2 | 356 |
| TFORF1550-TAZ | 356 |
| TFORF1938-MEIS1 | 356 |
| TFORF2123-ZNF273 | 356 |
| TFORF2219-YAP1 | 356 |
| TFORF2237-GATA4 | 356 |
| TFORF2244-TCF15 | 356 |
| TFORF2295-TAF11 | 356 |
| TFORF2301-HIC2 | 356 |
| TFORF2344-MITF | 356 |
| TFORF2537-SOX7 | 356 |
| TFORF2946-MYOD1 | 356 |
| TFORF2956-HNF4A | 356 |
| TFORF2960-PGR | 356 |
| TFORF3067-ZNF8 | 356 |
| TFORF3077-AP2B1 | 356 |
| TFORF3091-ZNF260 | 356 |
| TFORF3120-ZNF341 | 356 |
| TFORF3155-HLX | 356 |
| TFORF3161-ZNF433 | 356 |
| TFORF3162-ZNF253 | 356 |
| TFORF3206-TBR1 | 356 |
| TFORF3213-ZNF572 | 356 |
| TFORF3248-ZNF311 | 356 |
| TFORF3296-HIF1A | 356 |
| TFORF3307-ZNF563 | 356 |
| TFORF3310-ZNF561 | 356 |
| TFORF3359-ZNF263 | 356 |
| TFORF3365-FOXA1 | 356 |
| TFORF3415-NKRF | 356 |
| TFORF3432-NR3C1 | 356 |
| TFORF3451-ZNF366 | 356 |
| TFORF3473-RUNX1 | 356 |
| TFORF3487-NFATC1 | 356 |
| TFORF3508-ZNF134 | 356 |
| TFORF3519-TCF4 | 356 |
| TFORF3526-THAP12 | 356 |
| TFORF3536-HOXD4 | 356 |
| TFORF3549-GFP | 356 |
| TFORF3550-mCherry | 356 |
| TFORF0049-MAX | 357 |
| TFORF0977-ZNF121 | 357 |
| TFORF1388-PMS1 | 357 |
| TFORF1838-EZH2 | 357 |
| TFORF2106-ZBTB37 | 357 |
| TFORF2518-ZFP37 | 357 |
| TFORF3163-PPP1R13B | 357 |
| TFORF3277-ZNF518A | 357 |
| TFORF3409-ZNF621 | 357 |
| TFORF3413-ZFP82 | 357 |
| TFORF1162-NFKBIL1 | 358 |
| TFORF1867-POU2F2 | 358 |
| TFORF2600-POU6F2 | 358 |
| TFORF2897-MTA2 | 358 |
| TFORF2978-NROB2 | 358 |
| TFORF3039-NR1H3 | 358 |
| TFORF0302-CREM | 359 |
| TFORF0313-CREM | 359 |
| TFORF0531-PAX6 | 359 |
| TFORF0904-DNAJC1 | 359 |
| TFORF1384-ZNF788 | 359 |
| TFORF1496-HHEX | 359 |
| TFORF1800-USF2 | 359 |
| TFORF2044-ARID3C | 359 |
| TFORF3184-HSBP1 | 359 |
| TFORF3241-ZNF490 | 359 |
| TFORF3434-ZNF37A | 359 |
| TFORF3449-DMRT1 | 359 |
| TFORF0050-MAZ | 360 |
| TFORF0254-CAMTA1 | 360 |
| TFORF0416-ZNF559-ZNF177 | 360 |
| TFORF0735-ZNF480 | 360 |
| TFORF0850-ZIC4 | 360 |
| TFORF0882-HOXB1 | 360 |
| TFORF1004-POU1F1 | 360 |
| TFORF1047-SNAI2 | 360 |
| TFORF1068-NFATC2 | 360 |
| TFORF1241-LBX2 | 360 |
| TFORF1263-TP53 | 360 |
| TFORF1545-ZNF160 | 360 |
| TFORF1646-ZNF423 | 360 |
| TFORF1710-CBFB | 360 |
| TFORF1811-ZNF438 | 360 |
| TFORF2126-ZNF746 | 360 |
| TFORF2220-YAP1 | 360 |
| TFORF2415-ZNF268 | 360 |
| TFORF2619-ZXDB | 360 |
| TFORF2850-HMGA2 | 360 |
| TFORF3017-ETV4 | 360 |
| TFORF3288-HOXA9 | 360 |
| TFORF3408-ZNF286A | 360 |
| TFORF3438-GCM1 | 360 |
| TFORF0492-ZSCAN2 | 361 |
| TFORF1198-ZNF578 | 361 |
| TFORF1497-LYL1 | 361 |
| TFORF1821-ZNF138 | 361 |
| TFORF2028-NPAS1 | 361 |
| TFORF2517-PDCD2 | 361 |
| TFORF2815-FOSB | 361 |
| TFORF2851-HMGA2 | 361 |
| TFORF3301-OLIG3 | 361 |
| TFORF0407-CTCFL | 362 |
| TFORF1318-MKX | 362 |
| TFORF1451-DLX1 | 362 |
| TFORF1467-HOXD13 | 362 |
| TFORF1723-ZNF691 | 362 |
| TFORF2874-ELF1 | 362 |
| TFORF0410-CTCFL | 363 |
| TFORF0429-PBX1 | 363 |
| TFORF0623-DDIT3 | 363 |
| TFORF0760-OVOL1 | 363 |
| TFORF0823-LHX6 | 363 |
| TFORF0847-YBX3 | 363 |
| TFORF1127-THAP3 | 363 |
| TFORF1559-YAF2 | 363 |
| TFORF1722-DMRT3 | 363 |
| TFORF2385-MBD3 | 363 |
| TFORF2394-ZNF396 | 363 |
| TFORF2512-PDCD2 | 363 |
| TFORF2749-NKX6-3 | 363 |
| TFORF0309-CREM | 364 |
| TFORF1333-TFAP2D | 364 |
| TFORF1455-TFEC | 364 |
| TFORF1514-NKX3-1 | 364 |
| TFORF1866-POU2F2 | 364 |
| TFORF2001-MYEF2 | 364 |
| TFORF2122-ZNF563 | 364 |
| TFORF2242-GATA1 | 364 |
| TFORF2551-BID | 364 |
| TFORF3304-MITF | 364 |
| TFORF1457-TFEB | 365 |
| TFORF2341-MITF | 365 |
| TFORF2342-MITF | 365 |
| TFORF2345-MITF | 365 |
| TFORF2346-MITF | 365 |
| TFORF3303-MITF | 365 |
| TFORF3327-LHX6 | 365 |

TABLE 19B
Predicted double TFs for reference cell types from the human fetal cell atlas (42). Combinations were ranked based on the cell type-specific
gene signature score. Only the top ranked 100 possible combinations are shown. Combinations are presented as clusters from (A).

| | | Antigen presenting | | | | | | Bronchiolar and | | CCL19_CCL21 positive | |
|---|---|---|---|---|---|---|---|---|---|---|---|
| Amacrine cells | | cells | | Astrocytes | | Bipolar cells | | alveolar epithelial cells | | cells | |
| Clusters | Score | Clusters | Score | Clusters | Score | Clusters | Score | Clusters | Score | Clusters | Score |
| 57;243 | 5.96 | 156;270 | 4.43 | 208;327 | 7.96 | 31;92 | 5.02 | 99;261 | 2.81 | 299;336 | 9.48 |
| 58;293 | 5.96 | 69;98 | 3.46 | 95;327 | 7.96 | 34;106 | 5.02 | 97;261 | 2.81 | 192;299 | 8.32 |
| 53;256 | 4.93 | 79;97 | 3.46 | 327;362 | 6.62 | 27;245 | 3.94 | 96;261 | 2.81 | 250;281 | 7.22 |
| 79;256 | 4.93 | 79;138 | 3.46 | 169;327 | 6.62 | 49;180 | 3.94 | 139;261 | 2.81 | 299;338 | 7.22 |
| 248;256 | 4.93 | 58;80 | 3.46 | 187;327 | 6.62 | 50;225 | 3.94 | 163;261 | 2.81 | 282;298 | 7.22 |
| 17;256 | 4.93 | 98;156 | 3.46 | 320;327 | 6.62 | 38;154 | 3.94 | 142;261 | 2.81 | 178;282 | 7.22 |
| 51;293 | 4.93 | 73;98 | 3.46 | 197;327 | 6.62 | 29;101 | 3.94 | 153;353 | 2.81 | 203;282 | 7.22 |
| 59;256 | 4.93 | 78;138 | 3.46 | 265;327 | 6.62 | 27;254 | 3.94 | 325;354 | 2.81 | 299;337 | 7.22 |
| 72;256 | 3.96 | 98;180 | 3.46 | 181;327 | 6.62 | 100;253 | 3.94 | 153;354 | 2.81 | 281;362 | 7.22 |
| 121;293 | 3.96 | 98;169 | 3.46 | 170;327 | 6.62 | 55;68 | 3.94 | 74;354 | 2.81 | 175;282 | 7.22 |
| 73;256 | 3.96 | 98;182 | 3.46 | 207;327 | 6.62 | 34;93 | 3.94 | 95;261 | 2.81 | 185;282 | 7.22 |
| 256;348 | 3.96 | 49;214 | 2.58 | 68;327 | 6.62 | 31;164 | 3.94 | 143;261 | 2.81 | 282;300 | 7.22 |
| 65;243 | 3.96 | 49;162 | 2.58 | 325;327 | 6.62 | 92;254 | 3.94 | 88;331 | 1.83 | 280;303 | 6.17 |
| 190;256 | 3.96 | 98;206 | 2.58 | 206;327 | 6.62 | 34;68 | 3.94 | 161;261 | 1.83 | 300;364 | 6.17 |
| 184;225 | 3.96 | 98;214 | 2.58 | 247;327 | 6.62 | 29;267 | 3.94 | 61;354 | 1.83 | 212;281 | 6.17 |
| 144;256 | 3.96 | 49;216 | 2.58 | 171;327 | 6.62 | 92;239 | 3.94 | 198;261 | 1.83 | 93;300 | 6.17 |
| 121;256 | 3.96 | 35;71 | 2.58 | 164;327 | 6.62 | 29;244 | 3.94 | 249;354 | 1.83 | 281;338 | 6.17 |
| 69;256 | 3.96 | 219;326 | 2.58 | 298;327 | 6.62 | 29;103 | 3.94 | 7;261 | 1.83 | 61;282 | 6.17 |
| 58;288 | 3.96 | 35;270 | 2.58 | 19;327 | 6.62 | 29;254 | 3.94 | 171;354 | 1.83 | 137;282 | 6.17 |
| 58;256 | 3.96 | 77;138 | 2.58 | 233;327 | 6.62 | 118;234 | 3.94 | 231;316 | 1.83 | 282;345 | 6.17 |
| 217;249 | 3.96 | 156;314 | 2.58 | 165;327 | 6.62 | 34;208 | 3.94 | 216;354 | 1.83 | 282;299 | 6.17 |
| 66;293 | 3.96 | 77;150 | 2.58 | 271;327 | 6.62 | 2;230 | 3.94 | 111;261 | 1.83 | 146;282 | 6.17 |
| 94;229 | 3.96 | 77;192 | 2.58 | 192;327 | 6.62 | 34;118 | 3.94 | 73;354 | 1.83 | 151;281 | 6.17 |
| 249;256 | 3.96 | 61;138 | 2.58 | 194;327 | 6.62 | 32;254 | 3.94 | 130;261 | 1.83 | 193;282 | 6.17 |
| 70;249 | 3.96 | 98;193 | 2.58 | 185;327 | 6.62 | 32;179 | 3.94 | 156;261 | 1.83 | 250;282 | 6.17 |
| 63;249 | 3.96 | 98;216 | 2.58 | 73;327 | 6.62 | 32;208 | 3.94 | 74;170 | 1.83 | 152;282 | 6.17 |
| 58;249 | 3.96 | 79;169 | 2.58 | 220;327 | 6.62 | 34;105 | 3.94 | 312;328 | 1.83 | 282;315 | 6.17 |
| 64;256 | 3.96 | 107;138 | 2.58 | 195;327 | 6.62 | 32;359 | 2.96 | 41;261 | 1.83 | 151;282 | 6.17 |
| 242;256 | 3.96 | 138;294 | 2.58 | 236;327 | 6.62 | 116;236 | 2.96 | 51;261 | 1.83 | 193;299 | 6.17 |
| 63;256 | 3.96 | 138;323 | 2.58 | 237;327 | 6.62 | 230;253 | 2.96 | 239;261 | 1.83 | 282;302 | 6.17 |
| 195;256 | 3.96 | 79;150 | 2.58 | 64;327 | 6.62 | 32;362 | 2.96 | 216;261 | 1.83 | 303;338 | 6.17 |
| 41;256 | 3.96 | 314;335 | 2.58 | 193;327 | 6.62 | 27;291 | 2.96 | 25;261 | 1.83 | 202;299 | 6.17 |
| 215;256 | 3.96 | 138;359 | 2.58 | 203;327 | 6.62 | 33;51 | 2.96 | 88;318 | 1.83 | 202;282 | 6.17 |
| 229;256 | 3.96 | 80;96 | 2.58 | 58;327 | 6.62 | 27;208 | 2.96 | 82;261 | 1.83 | 282;338 | 6.17 |
| 253;287 | 3.96 | 80;219 | 2.58 | 210;327 | 6.62 | 51;254 | 2.96 | 88;354 | 1.83 | 299;347 | 6.17 |
| 42;293 | 3.96 | 138;245 | 2.58 | 327;350 | 6.62 | 33;71 | 2.96 | 312;332 | 1.83 | 199;282 | 6.17 |
| 125;293 | 3.96 | 98;152 | 2.58 | 327;352 | 6.62 | 27;220 | 2.96 | 16;261 | 1.83 | 299;350 | 6.17 |
| 71;256 | 3.96 | 150;213 | 2.58 | 178;327 | 6.62 | 29;266 | 2.96 | 184;354 | 1.83 | 277;299 | 6.17 |
| 195;213 | 3.96 | 62;203 | 2.58 | 131;327 | 6.62 | 29;262 | 2.96 | 160;261 | 1.83 | 4;282 | 6.17 |
| 16;242 | 3.96 | 150;207 | 2.58 | 204;327 | 6.62 | 92;118 | 2.96 | 237;354 | 1.83 | 72;282 | 6.17 |
| 75;256 | 3.96 | 138;223 | 2.58 | 327;363 | 6.62 | 92;121 | 2.96 | 172;323 | 1.83 | 73;282 | 6.17 |
| 65;256 | 3.96 | 98;147 | 2.58 | 327;360 | 6.62 | 54;223 | 2.96 | 135;261 | 1.83 | 281;298 | 6.17 |
| 98;256 | 3.96 | 135;360 | 2.58 | 327;336 | 6.62 | 130;352 | 2.96 | 197;261 | 1.83 | 267;299 | 6.17 |
| 74;293 | 3.96 | 138;180 | 2.58 | 9;327 | 5.38 | 32;323 | 2.96 | 130;354 | 1.83 | 217;282 | 6.17 |
| 42;256 | 3.96 | 138;183 | 2.58 | 302;327 | 5.38 | 27;223 | 2.96 | 20;261 | 1.83 | 207;299 | 6.17 |
| 216;256 | 3.96 | 87;138 | 2.58 | 167;327 | 5.38 | 32;262 | 2.96 | 152;261 | 1.83 | 176;282 | 6.17 |
| 195;297 | 3.96 | 75;97 | 2.58 | 215;327 | 5.38 | 32;267 | 2.96 | 122;261 | 1.83 | 60;281 | 6.17 |
| 68;293 | 3.96 | 17;80 | 2.58 | 267;327 | 5.38 | 27;230 | 2.96 | 157;261 | 1.83 | 175;278 | 6.17 |
| 62;242 | 3.96 | 206;270 | 2.58 | 316;327 | 5.38 | 27;234 | 2.96 | 224;261 | 1.83 | 2;282 | 6.17 |
| 132;293 | 3.96 | 98;364 | 2.58 | 238;327 | 5.38 | 27;225 | 2.96 | 33;261 | 1.83 | 299;325 | 5.18 |
| 143;256 | 3.96 | 59;78 | 2.58 | 239;327 | 5.38 | 27;236 | 2.96 | 120;261 | 1.83 | 210;304 | 5.18 |
| 18;293 | 3.96 | 169;270 | 2.58 | 101;327 | 5.38 | 27;239 | 2.96 | 68;261 | 1.83 | 282;304 | 5.18 |
| 67;228 | 3.96 | 53;97 | 2.58 | 266;333 | 5.38 | 38;117 | 2.96 | 103;261 | 1.83 | 147;300 | 5.18 |
| 56;293 | 3.96 | 12;150 | 1.79 | 272;327 | 5.38 | 230;308 | 2.96 | 132;354 | 1.83 | 299;330 | 5.18 |
| 256;324 | 3.96 | 79;182 | 1.79 | 14;327 | 5.38 | 51;253 | 2.96 | 61;171 | 1.83 | 192;300 | 5.18 |
| 228;256 | 3.96 | 96;351 | 1.79 | 59;327 | 5.38 | 33;92 | 2.96 | 82;354 | 1.83 | 300;315 | 5.18 |
| 66;256 | 3.96 | 73;99 | 1.79 | 147;327 | 5.38 | 130;290 | 2.96 | 147;354 | 1.83 | 104;299 | 5.18 |
| 110;256 | 3.96 | 45;277 | 1.79 | 254;327 | 5.38 | 54;247 | 2.96 | 106;261 | 1.83 | 52;299 | 5.18 |
| 111;297 | 3.96 | 75;138 | 1.79 | 234;327 | 5.38 | 92;94 | 2.96 | 61;261 | 1.83 | 300;316 | 5.18 |
| 20;256 | 3.96 | 45;294 | 1.79 | 266;324 | 5.38 | 85;92 | 2.96 | 30;261 | 1.83 | 300;334 | 5.18 |
| 142;256 | 3.96 | 138;264 | 1.79 | 11;327 | 5.38 | 38;119 | 2.96 | 144;354 | 1.83 | 105;299 | 5.18 |
| 65;293 | 3.96 | 270;282 | 1.79 | 266;327 | 5.38 | 29;290 | 2.96 | 73;261 | 1.83 | 300;340 | 5.18 |
| 155;256 | 3.96 | 45;214 | 1.79 | 327;348 | 5.38 | 144;239 | 2.96 | 61;173 | 1.83 | 300;345 | 5.18 |
| 62;256 | 3.96 | 97;138 | 1.79 | 213;327 | 5.38 | 54;241 | 2.96 | 94;261 | 1.83 | 299;364 | 5.18 |
| 67;256 | 3.96 | 79;206 | 1.79 | 295;327 | 5.38 | 34;214 | 2.96 | 217;261 | 1.83 | 280;302 | 5.18 |
| 59;297 | 3.96 | 97;137 | 1.79 | 244;327 | 5.38 | 54;213 | 2.96 | 262;330 | 1.83 | 106;281 | 5.18 |
| 19;256 | 3.96 | 97;135 | 1.79 | 262;327 | 5.38 | 2;54 | 2.96 | 172;261 | 1.83 | 300;361 | 5.18 |
| 187;256 | 3.96 | 107;150 | 1.79 | 35;327 | 5.38 | 21;51 | 2.96 | 238;261 | 1.83 | 280;300 | 5.18 |
| 233;256 | 3.96 | 97;131 | 1.79 | 288;327 | 5.38 | 34;359 | 2.96 | 185;261 | 1.83 | 252;281 | 5.18 |
| 247;293 | 3.96 | 107;162 | 1.79 | 139;327 | 5.38 | 29;201 | 2.96 | 200;261 | 1.83 | 17;281 | 5.18 |
| 178;256 | 3.96 | 97;128 | 1.79 | 214;327 | 5.38 | 34;355 | 2.96 | 261;342 | 1.83 | 92;299 | 5.18 |
| 18;256 | 3.96 | 91;228 | 1.79 | 107;327 | 5.38 | 29;194 | 2.96 | 137;149 | 1.83 | 161;303 | 5.18 |
| 67;249 | 3.96 | 45;302 | 1.79 | 172;327 | 5.38 | 29;193 | 2.96 | 261;343 | 1.83 | 202;281 | 5.18 |
| 184;242 | 3.96 | 138;168 | 1.79 | 264;327 | 5.38 | 29;192 | 2.96 | 42;132 | 1.83 | 225;281 | 5.18 |
| 189;293 | 3.07 | 46;150 | 1.79 | 62;327 | 5.38 | 100;323 | 2.96 | 25;354 | 1.83 | 210;299 | 5.18 |
| 65;248 | 3.07 | 58;162 | 1.79 | 241;327 | 5.38 | 33;225 | 2.96 | 261;346 | 1.83 | 302;338 | 5.18 |
| 154;293 | 3.07 | 138;197 | 1.79 | 135;327 | 5.38 | 34;351 | 2.96 | 261;347 | 1.83 | 175;357 | 5.18 |
| 136;256 | 3.07 | 138;196 | 1.79 | 296;327 | 5.38 | 92;176 | 2.96 | 261;348 | 1.83 | 73;299 | 5.18 |
| 165;256 | 3.07 | 138;195 | 1.79 | 327;356 | 5.38 | 100;346 | 2.96 | 62;261 | 1.83 | 175;361 | 5.18 |
| 51;256 | 3.07 | 94;150 | 1.79 | 242;327 | 5.38 | 103;218 | 2.96 | 215;261 | 1.83 | 175;364 | 5.18 |
| 14;256 | 3.07 | 73;95 | 1.79 | 182;327 | 5.38 | 54;215 | 2.96 | 202;261 | 1.83 | 207;280 | 5.18 |
| 160;293 | 3.07 | 270;365 | 1.79 | 327;354 | 5.38 | 33;253 | 2.96 | 15;261 | 1.83 | 195;282 | 5.18 |
| 16;293 | 3.07 | 138;187 | 1.79 | 160;327 | 5.38 | 29;179 | 2.96 | 261;350 | 1.83 | 74;281 | 5.18 |
| 131;256 | 3.07 | 79;80 | 1.79 | 198;327 | 5.38 | 34;324 | 2.96 | 249;261 | 1.83 | 330;345 | 5.18 |
| 131;293 | 3.07 | 138;184 | 1.79 | 103;327 | 5.38 | 103;197 | 2.96 | 214;354 | 1.83 | 282;363 | 5.18 |
| 97;256 | 3.07 | 138;143 | 1.79 | 320;321 | 5.38 | 29;174 | 2.96 | 162;261 | 1.83 | 282;361 | 5.18 |
| 127;308 | 3.07 | 138;182 | 1.79 | 275;327 | 5.38 | 29;173 | 2.96 | 261;335 | 1.83 | 208;282 | 5.18 |
| 195;206 | 3.07 | 138;179 | 1.79 | 228;327 | 5.38 | 29;172 | 2.96 | 261;336 | 1.83 | 331;338 | 5.18 |
| 58;308 | 3.07 | 138;177 | 1.79 | 6;327 | 5.38 | 29;171 | 2.96 | 261;337 | 1.83 | 204;300 | 5.18 |
| 217;292 | 3.07 | 73;80 | 1.79 | 163;327 | 5.38 | 29;170 | 2.96 | 129;261 | 1.83 | 85;356 | 5.18 |
| 195;215 | 3.07 | 78;335 | 1.79 | 7;327 | 5.38 | 1;246 | 2.96 | 240;261 | 1.83 | 283;336 | 5.18 |
| 72;249 | 3.07 | 138;166 | 1.79 | 229;327 | 5.38 | 239;342 | 2.96 | 203;261 | 1.83 | 175;281 | 5.18 |
| 151;247 | 3.07 | 138;204 | 1.79 | 225;327 | 5.38 | 34;360 | 2.96 | 261;339 | 1.83 | 197;281 | 5.18 |
| 58;227 | 3.07 | 138;206 | 1.79 | 143;327 | 5.38 | 46;243 | 2.96 | 261;340 | 1.83 | 283;347 | 5.18 |
| 182;293 | 3.07 | 23;138 | 1.79 | 157;327 | 5.38 | 54;171 | 2.96 | 261;341 | 1.83 | 206;282 | 5.18 |
| 177;195 | 3.07 | 64;314 | 1.79 | 211;327 | 5.38 | 46;239 | 2.96 | 49;261 | 1.83 | 175;242 | 5.18 |
| 59;209 | 3.07 | 97;112 | 1.79 | 226;327 | 5.38 | 54;214 | 2.96 | 261;351 | 1.83 | 16;282 | 5.18 |
| 35;58 | 3.07 | 214;314 | 1.79 | 18;327 | 5.38 | 38;128 | 2.96 | 50;161 | 1.83 | 206;299 | 5.18 |
| 204;256 | 3.07 | 58;138 | 1.79 | 152;327 | 5.38 | 32;241 | 2.96 | 261;364 | 1.83 | 208;300 | 5.18 |
| 151;293 | 3.07 | 138;219 | 1.79 | 127;327 | 5.38 | 92;136 | 2.96 | 171;261 | 1.83 | 166;299 | 5.18 |

| | | | | | | | | | | Corneal and | |
|---|---|---|---|---|---|---|---|---|---|---|---|
| | | CSH1_CSH2 positive | | | | | | | | conjunctival epithelial | |
| CLC_IL5RA positive cells | | cells | | Cardiomyocytes | | Chromaffin cells | | Ciliated epithelial cells | | cells | |
| Clusters | Score | Clusters | Score | Clusters | Score | Clusters | Score | Clusters | Score | Clusters | Score |
| 175;331 | 12.73 | 151;273 | 9.92 | 175;331 | 13.76 | 254;316 | 5.19 | 259;343 | 25.41 | 80;357 | 6.03 |
| 280;331 | 9.95 | 10;273 | 9.92 | 210;280 | 12.22 | 254;315 | 5.19 | 154;259 | 25.41 | 273;334 | 6.03 |
| 162;280 | 9.95 | 67;273 | 9.92 | 150;280 | 12.22 | 62;254 | 3.93 | 162;259 | 25.41 | 170;273 | 6.03 |
| 150;280 | 9.95 | 200;273 | 9.92 | 162;280 | 12.22 | 79;254 | 3.93 | 91;259 | 25.41 | 156;273 | 6.03 |
| 280;363 | 9.95 | 60;273 | 9.92 | 280;363 | 12.22 | 213;254 | 3.93 | 252;259 | 23.38 | 150;273 | 6.03 |
| 211;280 | 9.95 | 182;273 | 8.53 | 211;280 | 12.22 | 197;254 | 3.93 | 89;259 | 23.38 | 80;156 | 6.03 |
| 210;280 | 9.95 | 273;316 | 8.53 | 171;280 | 10.75 | 86;254 | 3.93 | 259;342 | 23.38 | 164;273 | 6.03 |
| 298;331 | 9.95 | 146;273 | 8.53 | 197;280 | 10.75 | 201;254 | 3.93 | 155;259 | 23.38 | 80;334 | 6.03 |
| 280;321 | 9.95 | 154;273 | 8.53 | 280;338 | 10.75 | 61;254 | 3.93 | 163;259 | 23.38 | 11;273 | 6.03 |
| 180;280 | 8.66 | 68;273 | 8.53 | 172;280 | 10.75 | 223;254 | 3.93 | 259;265 | 23.38 | 80;203 | 4.75 |
| 206;280 | 8.66 | 128;273 | 8.53 | 195;280 | 10.75 | 254;351 | 3.93 | 157;259 | 23.38 | 173;273 | 4.75 |
| 202;331 | 8.66 | 152;273 | 8.53 | 146;280 | 10.75 | 129;254 | 3.93 | 153;259 | 23.38 | 80;200 | 4.75 |
| 146;280 | 8.66 | 18;273 | 8.53 | 159;280 | 10.75 | 64;254 | 3.93 | 138;259 | 23.38 | 80;193 | 4.75 |
| 280;309 | 8.66 | 246;273 | 8.53 | 103;280 | 10.75 | 237;254 | 3.93 | 259;268 | 23.38 | 80;194 | 4.75 |
| 176;280 | 8.66 | 273;338 | 8.53 | 182;280 | 10.75 | 254;270 | 3.93 | 106;259 | 23.38 | 105;273 | 4.75 |
| 159;281 | 8.66 | 273;353 | 8.53 | 185;280 | 10.75 | 32;254 | 3.93 | 118;259 | 23.38 | 175;283 | 4.75 |
| 85;280 | 8.66 | 15;273 | 8.53 | 178;280 | 10.75 | 128;254 | 3.93 | 221;259 | 23.38 | 80;238 | 4.75 |
| 185;280 | 8.66 | 13;273 | 8.53 | 102;280 | 10.75 | 210;254 | 3.93 | 209;259 | 23.38 | 283;361 | 4.75 |
| 152;280 | 8.66 | 93;273 | 8.53 | 205;280 | 10.75 | 173;254 | 3.93 | 186;259 | 23.38 | 178;273 | 4.75 |
| 280;310 | 8.66 | 157;273 | 8.53 | 280;320 | 10.75 | 238;254 | 3.93 | 259;352 | 23.38 | 80;224 | 4.75 |
| 12;280 | 8.66 | 171;273 | 8.53 | 280;355 | 10.75 | 217;254 | 3.93 | 259;260 | 23.38 | 80;178 | 4.75 |
| 182;280 | 8.66 | 76;273 | 8.53 | 147;280 | 10.75 | 45;254 | 3.93 | 131;259 | 23.38 | 80;154 | 4.75 |
| 217;280 | 8.66 | 168;273 | 8.53 | 298;331 | 10.75 | 2;254 | 3.93 | 132;259 | 23.38 | 172;283 | 4.75 |
| 159;280 | 8.66 | 214;273 | 8.53 | 280;364 | 10.75 | 254;362 | 3.93 | 34;259 | 23.38 | 194;273 | 4.75 |
| 171;280 | 8.66 | 231;273 | 8.53 | 62;280 | 10.75 | 178;254 | 3.93 | 187;259 | 23.38 | 249;273 | 4.75 |
| 89;280 | 8.66 | 160;273 | 8.53 | 280;331 | 10.75 | 253;316 | 3.93 | 256;259 | 23.38 | 193;272 | 4.75 |
| 149;280 | 8.66 | 156;273 | 8.53 | 161;280 | 10.75 | 85;254 | 3.93 | 52;259 | 23.38 | 193;273 | 4.75 |
| 103;280 | 8.66 | 81;273 | 8.53 | 280;296 | 10.75 | 253;315 | 3.93 | 108;259 | 23.38 | 113;283 | 4.75 |
| 167;331 | 8.66 | 11;273 | 8.53 | 159;281 | 10.75 | 88;254 | 3.93 | 139;259 | 23.38 | 197;273 | 4.75 |
| 175;280 | 8.66 | 130;273 | 8.53 | 280;310 | 10.75 | 171;254 | 3.93 | 137;259 | 23.38 | 167;283 | 4.75 |
| 72;280 | 8.66 | 273;360 | 8.53 | 180;280 | 10.75 | 174;254 | 3.93 | 259;341 | 21.45 | 172;273 | 4.75 |
| 205;280 | 8.66 | 229;273 | 8.53 | 280;352 | 10.75 | 183;254 | 3.93 | 143;259 | 21.45 | 171;283 | 4.75 |
| 280;296 | 8.66 | 105;273 | 8.53 | 152;280 | 10.75 | 182;254 | 3.93 | 259;339 | 21.45 | 171;273 | 4.75 |
| 62;280 | 8.66 | 43;273 | 8.53 | 149;280 | 10.75 | 206;254 | 3.93 | 70;259 | 21.45 | 80;164 | 4.75 |
| 280;291 | 8.66 | 60;274 | 8.53 | 175;280 | 9.37 | 254;363 | 3.93 | 189;259 | 21.45 | 170;283 | 4.75 |
| 161;280 | 8.66 | 212;273 | 8.53 | 152;281 | 9.37 | 68;254 | 3.93 | 227;259 | 21.45 | 80;150 | 4.75 |
| 195;280 | 8.66 | 211;273 | 8.53 | 68;280 | 9.37 | 15;254 | 3.93 | 234;259 | 21.45 | 170;272 | 4.75 |
| 173;280 | 8.66 | 148;273 | 8.53 | 280;298 | 9.37 | 169;254 | 3.93 | 148;259 | 21.45 | 80;113 | 4.75 |
| 135;280 | 8.66 | 167;273 | 8.53 | 72;280 | 9.37 | 177;254 | 3.93 | 3;259 | 21.45 | 169;273 | 4.75 |
| 183;280 | 8.66 | 189;273 | 8.53 | 135;280 | 9.37 | 92;254 | 3.93 | 156;259 | 21.45 | 80;96 | 4.75 |
| 280;364 | 8.66 | 249;273 | 8.53 | 108;280 | 9.37 | 214;254 | 3.93 | 259;262 | 21.45 | 273;294 | 4.75 |
| 214;280 | 8.66 | 147;273 | 8.53 | 203;281 | 9.37 | 205;254 | 3.93 | 104;259 | 21.45 | 273;298 | 4.75 |
| 153;280 | 8.66 | 207;273 | 8.53 | 265;280 | 9.37 | 13;254 | 3.93 | 188;259 | 21.45 | 93;185 | 4.75 |
| 177;280 | 8.66 | 203;273 | 8.53 | 281;298 | 9.37 | 152;254 | 3.93 | 80;259 | 21.45 | 93;164 | 4.75 |
| 153;281 | 8.66 | 273;355 | 8.53 | 280;282 | 9.37 | 254;269 | 3.93 | 245;259 | 21.45 | 93;156 | 4.75 |
| 147;280 | 8.66 | 175;273 | 8.53 | 196;280 | 9.37 | 215;254 | 3.93 | 83;259 | 21.45 | 273;338 | 4.75 |
| 178;280 | 8.66 | 61;273 | 8.53 | 215;280 | 9.37 | 71;254 | 3.93 | 259;338 | 21.45 | 273;348 | 4.75 |
| 265;280 | 8.66 | 169;273 | 8.53 | 280;300 | 9.37 | 195;254 | 3.93 | 84;259 | 21.45 | 263;273 | 4.75 |
| 11;280 | 8.66 | 60;207 | 7.22 | 174;280 | 9.37 | 196;254 | 3.93 | 77;259 | 21.45 | 273;351 | 4.75 |
| 129;280 | 8.66 | 4;273 | 7.22 | 154;280 | 9.37 | 254;291 | 3.93 | 242;259 | 21.45 | 271;334 | 4.75 |
| 280;355 | 8.66 | 56;273 | 7.22 | 254;280 | 9.37 | 254;320 | 3.93 | 259;307 | 21.45 | 271;284 | 4.75 |
| 172;280 | 8.66 | 273;351 | 7.22 | 280;325 | 9.37 | 254;295 | 3.93 | 259;306 | 21.45 | 270;273 | 4.75 |
| 280;352 | 8.66 | 129;273 | 7.22 | 214;280 | 9.37 | 166;254 | 3.93 | 142;259 | 21.45 | 93;361 | 4.75 |
| 197;280 | 8.66 | 20;273 | 7.22 | 206;280 | 9.37 | 203;254 | 3.93 | 150;259 | 21.45 | 93;355 | 4.75 |
| 213;280 | 8.66 | 273;350 | 7.22 | 175;296 | 9.37 | 146;254 | 3.93 | 38;259 | 21.45 | 93;334 | 4.75 |
| 186;280 | 8.66 | 205;273 | 7.22 | 153;281 | 9.37 | 168;254 | 3.93 | 66;259 | 21.45 | 93;284 | 4.75 |
| 280;338 | 8.66 | 238;273 | 7.22 | 153;280 | 9.37 | 172;254 | 3.93 | 258;259 | 21.45 | 93;270 | 4.75 |
| 157;280 | 8.66 | 103;273 | 7.22 | 173;280 | 9.37 | 186;254 | 3.93 | 31;259 | 21.45 | 89;283 | 4.75 |
| 179;280 | 8.66 | 273;354 | 7.22 | 18;280 | 9.37 | 193;254 | 3.93 | 171;259 | 21.45 | 35;273 | 4.75 |
| 280;325 | 8.66 | 197;273 | 7.22 | 129;280 | 9.37 | 216;254 | 3.93 | 116;259 | 21.45 | 35;283 | 4.75 |
| 175;296 | 8.66 | 165;273 | 7.22 | 280;321 | 9.37 | 147;254 | 3.93 | 259;305 | 21.45 | 283;308 | 4.75 |
| 280;361 | 8.66 | 9;273 | 7.22 | 280;291 | 9.37 | 254;325 | 3.93 | 82;259 | 21.45 | 283;321 | 4.75 |
| 196;280 | 8.66 | 208;273 | 7.22 | 12;280 | 9.37 | 12;254 | 3.93 | 159;259 | 21.45 | 80;360 | 4.75 |
| 102;280 | 8.66 | 163;273 | 7.22 | 2;280 | 9.37 | 207;254 | 3.93 | 166;259 | 21.45 | 80;355 | 4.75 |
| 215;280 | 8.66 | 273;310 | 7.22 | 156;280 | 9.37 | 202;254 | 3.93 | 128;259 | 21.45 | 283;331 | 4.75 |
| 280;319 | 8.66 | 166;273 | 7.22 | 11;280 | 9.37 | 192;254 | 3.93 | 165;259 | 21.45 | 283;338 | 4.75 |
| 280;320 | 8.66 | 1;273 | 7.22 | 9;280 | 9.37 | 185;254 | 3.93 | 93;259 | 21.45 | 73;273 | 4.75 |
| 154;280 | 8.66 | 213;273 | 7.22 | 280;309 | 9.37 | 236;254 | 3.93 | 110;259 | 21.45 | 283;355 | 4.75 |
| 94;280 | 8.66 | 215;273 | 7.22 | 183;280 | 9.37 | 208;254 | 3.93 | 119;259 | 21.45 | 98;273 | 4.75 |
| 280;359 | 8.66 | 145;273 | 7.22 | 176;280 | 9.37 | 254;309 | 3.93 | 129;259 | 21.45 | 35;272 | 4.75 |
| 2;280 | 7.43 | 273;318 | 7.22 | 179;280 | 9.37 | 253;271 | 2.81 | 144;259 | 21.45 | 83;273 | 4.75 |
| 281;319 | 7.43 | 273;320 | 7.22 | 94;280 | 9.37 | 214;253 | 2.81 | 185;259 | 21.45 | 185;273 | 4.75 |
| 175;219 | 7.43 | 198;273 | 7.22 | 192;280 | 9.37 | 150;254 | 2.81 | 164;259 | 21.45 | 273;361 | 4.75 |
| 280;282 | 7.43 | 273;321 | 7.22 | 208;280 | 9.37 | 135;254 | 2.81 | 259;348 | 21.45 | 273;363 | 4.75 |
| 166;280 | 7.43 | 41;273 | 7.22 | 166;280 | 9.37 | 241;254 | 2.81 | 90;259 | 21.45 | 24;283 | 4.75 |
| 103;331 | 7.43 | 242;273 | 7.22 | 177;280 | 9.37 | 153;237 | 2.81 | 259;336 | 21.45 | 188;283 | 4.75 |
| 149;281 | 7.43 | 273;322 | 7.22 | 85;280 | 9.37 | 74;254 | 2.81 | 24;259 | 21.45 | 85;283 | 4.75 |
| 128;280 | 7.43 | 177;273 | 7.22 | 217;280 | 9.37 | 117;254 | 2.81 | 241;259 | 21.45 | 97;273 | 4.75 |
| 74;280 | 7.43 | 155;273 | 7.22 | 86;280 | 9.37 | 1;254 | 2.81 | 107;259 | 21.45 | 23;273 | 4.75 |
| 156;280 | 7.43 | 121;273 | 7.22 | 157;280 | 9.37 | 202;253 | 2.81 | 259;362 | 21.45 | 35;93 | 4.75 |
| 64;280 | 7.43 | 273;323 | 7.22 | 207;280 | 9.37 | 182;253 | 2.81 | 211;259 | 21.45 | 11;80 | 4.75 |
| 18;280 | 7.43 | 202;273 | 7.22 | 151;281 | 9.37 | 247;252 | 2.81 | 259;365 | 21.45 | 46;273 | 4.75 |
| 55;282 | 7.43 | 30;273 | 7.22 | 169;280 | 9.37 | 253;308 | 2.81 | 173;259 | 21.45 | 46;271 | 4.75 |
| 153;330 | 7.43 | 79;273 | 7.22 | 202;331 | 9.37 | 65;254 | 2.81 | 56;259 | 21.45 | 57;273 | 4.75 |
| 55;281 | 7.43 | 95;273 | 7.22 | 222;280 | 9.37 | 26;254 | 2.81 | 200;259 | 21.45 | 63;273 | 4.75 |
| 4;280 | 7.43 | 139;273 | 7.22 | 155;280 | 9.37 | 26;253 | 2.81 | 127;259 | 21.45 | 46;80 | 4.75 |
| 207;280 | 7.43 | 186;273 | 7.22 | 280;362 | 9.37 | 103;253 | 2.81 | 167;259 | 21.45 | 155;272 | 4.75 |
| 280;362 | 7.43 | 153;273 | 7.22 | 97;280 | 9.37 | 103;254 | 2.81 | 212;259 | 21.45 | 135;273 | 4.75 |
| 186;281 | 7.43 | 196;273 | 7.22 | 216;280 | 9.37 | 88;253 | 2.81 | 202;259 | 21.45 | 119;283 | 4.75 |
| 175;330 | 7.43 | 138;273 | 7.22 | 213;280 | 9.37 | 54;248 | 2.81 | 210;259 | 21.45 | 155;273 | 4.75 |
| 155;280 | 7.43 | 273;346 | 7.22 | 167;331 | 9.37 | 72;254 | 2.81 | 259;318 | 21.45 | 215;273 | 4.75 |
| 175;323 | 7.43 | 2;273 | 7.22 | 280;361 | 9.37 | 67;253 | 2.81 | 259;316 | 21.45 | 214;273 | 4.75 |
| 280;330 | 7.43 | 42;273 | 7.22 | 186;280 | 9.37 | 67;252 | 2.81 | 174;259 | 21.45 | 156;272 | 4.75 |
| 169;280 | 7.43 | 60;358 | 7.22 | 280;359 | 9.37 | 154;254 | 2.81 | 259;291 | 21.45 | 150;283 | 4.75 |
| 68;280 | 7.43 | 273;340 | 7.22 | 149;281 | 9.37 | 117;253 | 2.81 | 113;226 | 21.45 | 208;283 | 4.75 |
| 153;282 | 7.43 | 170;273 | 7.22 | 151;280 | 9.37 | 15;253 | 2.81 | 259;315 | 21.45 | 156;283 | 4.75 |
| 175;355 | 7.43 | 135;273 | 7.22 | 89;280 | 9.37 | 225;297 | 2.81 | 244;259 | 21.45 | 154;283 | 4.75 |
| 178;331 | 7.43 | 94;273 | 7.22 | 18;281 | 8.05 | 191;254 | 2.81 | 259;285 | 21.45 | 66;273 | 4.75 |
| 240;280 | 7.43 | 225;273 | 7.22 | 280;343 | 8.05 | 89;253 | 2.81 | 120;259 | 21.45 | 207;273 | 4.75 |
| 217;331 | 7.43 | 273;339 | 7.22 | 128;280 | 8.05 | 185;253 | 2.81 | 259;286 | 21.45 | 206;283 | 4.75 |

| ELF3_AGBL2 positive | | | | | | | | | | | |
|---|---|---|---|---|---|---|---|---|---|---|---|
| cells | | ENS glia | | ENS neurons | | Endocardial cells | | Epicardial fat cells | | Erythroblasts | |
| Clusters | Score | Clusters | Score | Clusters | Score | Clusters | Score | Clusters | Score | Clusters | Score |
| 175;331 | 13.76 | 153;336 | 4.23 | 251;303 | 4.86 | 305;315 | 10.54 | 55;282 | 4.60 | 158;165 | 3.31 |
| 210;280 | 12.22 | 155;337 | 3.19 | 251;287 | 3.81 | 16;304 | 9.27 | 280;340 | 3.30 | 106;161 | 3.31 |
| 150;280 | 12.22 | 193;362 | 3.19 | 210;254 | 3.81 | 165;305 | 9.27 | 33;282 | 3.30 | 19;69 | 3.31 |
| 162;280 | 12.22 | 107;242 | 3.19 | 251;293 | 3.81 | 175;304 | 9.27 | 149;303 | 3.30 | 155;158 | 3.31 |
| 280;363 | 12.22 | 65;198 | 3.19 | 155;251 | 3.81 | 230;305 | 9.27 | 133;280 | 3.30 | 131;158 | 3.31 |
| 211;280 | 12.22 | 65;202 | 3.19 | 101;251 | 3.81 | 305;330 | 9.27 | 118;299 | 3.30 | 19;210 | 3.31 |
| 171;280 | 10.75 | 65;227 | 3.19 | 251;260 | 3.81 | 299;304 | 9.27 | 55;356 | 3.30 | 158;364 | 2.34 |
| 197;280 | 10.75 | 107;357 | 3.19 | 167;178 | 3.81 | 305;331 | 9.27 | 280;339 | 3.30 | 158;362 | 2.34 |
| 280;338 | 10.75 | 190;336 | 3.19 | 150;251 | 3.81 | 304;363 | 8.05 | 55;280 | 3.30 | 158;223 | 2.34 |
| 172;280 | 10.75 | 190;337 | 3.19 | 220;252 | 3.81 | 245;304 | 8.05 | 21;198 | 2.16 | 158;222 | 2.34 |
| 195;280 | 10.75 | 65;334 | 3.19 | 220;251 | 3.81 | 72;305 | 8.05 | 95;149 | 2.16 | 107;156 | 2.34 |
| 146;280 | 10.75 | 65;335 | 3.19 | 9;250 | 3.81 | 8;304 | 8.05 | 329;342 | 2.16 | 61;169 | 2.34 |
| 159;280 | 10.75 | 193;363 | 3.19 | 119;251 | 3.81 | 304;346 | 8.05 | 329;341 | 2.16 | 84;323 | 2.34 |
| 103;280 | 10.75 | 212;336 | 3.19 | 251;313 | 3.81 | 171;305 | 8.05 | 95;329 | 2.16 | 158;325 | 2.34 |
| 182;280 | 10.75 | 250;321 | 2.25 | 160;251 | 3.81 | 304;333 | 8.05 | 95;296 | 2.16 | 157;165 | 2.34 |
| 185;280 | 10.75 | 65;262 | 2.25 | 193;254 | 3.81 | 304;361 | 8.05 | 95;285 | 2.16 | 158;178 | 2.34 |
| 178;280 | 10.75 | 222;362 | 2.25 | 201;251 | 3.81 | 203;304 | 8.05 | 282;361 | 2.16 | 71;158 | 2.34 |
| 102;280 | 10.75 | 53;337 | 2.25 | 251;364 | 3.81 | 185;304 | 8.05 | 21;280 | 2.16 | 158;365 | 2.34 |
| 205;280 | 10.75 | 316;342 | 2.25 | 63;251 | 3.81 | 286;305 | 8.05 | 21;356 | 2.16 | 158;323 | 2.34 |
| 280;320 | 10.75 | 195;338 | 2.25 | 251;295 | 3.81 | 180;304 | 8.05 | 131;280 | 2.16 | 169;205 | 2.34 |
| 280;355 | 10.75 | 223;250 | 2.25 | 251;343 | 2.85 | 298;304 | 8.05 | 171;280 | 2.16 | 169;218 | 2.34 |
| 147;280 | 10.75 | 65;284 | 2.25 | 204;251 | 2.85 | 188;304 | 8.05 | 170;280 | 2.16 | 106;158 | 2.34 |
| 298;331 | 10.75 | 65;280 | 2.25 | 251;346 | 2.85 | 304;322 | 8.05 | 146;280 | 2.16 | 326;328 | 2.34 |
| 280;364 | 10.75 | 172;348 | 2.25 | 251;340 | 2.85 | 190;304 | 8.05 | 74;280 | 2.16 | 229;326 | 2.34 |
| 62;280 | 10.75 | 223;336 | 2.25 | 251;348 | 2.85 | 302;304 | 8.05 | 286;324 | 2.16 | 106;155 | 2.34 |
| 280;331 | 10.75 | 146;250 | 2.25 | 251;354 | 2.85 | 304;305 | 8.05 | 286;299 | 2.16 | 130;330 | 2.34 |
| 161;280 | 10.75 | 107;350 | 2.25 | 94;253 | 2.85 | 196;305 | 8.05 | 129;280 | 2.16 | 158;184 | 2.34 |
| 280;296 | 10.75 | 89;193 | 2.25 | 94;251 | 2.85 | 305;322 | 8.05 | 129;282 | 2.16 | 169;206 | 2.34 |
| 159;281 | 10.75 | 39;349 | 2.25 | 204;254 | 2.85 | 230;304 | 8.05 | 130;280 | 2.16 | 158;225 | 2.34 |
| 280;310 | 10.75 | 173;250 | 2.25 | 251;350 | 2.85 | 214;305 | 8.05 | 47;280 | 2.16 | 146;324 | 2.34 |
| 180;280 | 10.75 | 107;338 | 2.25 | 251;322 | 2.85 | 148;304 | 8.05 | 96;280 | 2.16 | 18;158 | 2.34 |
| 280;352 | 10.75 | 65;252 | 2.25 | 97;251 | 2.85 | 165;304 | 8.05 | 285;303 | 2.16 | 167;206 | 2.34 |
| 152;280 | 10.75 | 65;251 | 2.25 | 167;251 | 2.85 | 82;304 | 8.05 | 285;299 | 2.16 | 46;158 | 2.34 |
| 149;280 | 10.75 | 65;250 | 2.25 | 95;193 | 2.85 | 224;304 | 8.05 | 190;280 | 2.16 | 69;240 | 2.34 |
| 175;280 | 9.37 | 173;338 | 2.25 | 251;336 | 2.85 | 227;305 | 8.05 | 132;280 | 2.16 | 158;309 | 2.34 |
| 152;281 | 9.37 | 251;337 | 2.25 | 251;335 | 2.85 | 55;304 | 8.05 | 186;280 | 2.16 | 158;294 | 2.34 |
| 68;280 | 9.37 | 65;292 | 2.25 | 251;334 | 2.85 | 237;304 | 8.05 | 134;285 | 2.16 | 158;163 | 2.34 |
| 280;298 | 9.37 | 146;336 | 2.25 | 251;330 | 2.85 | 305;336 | 8.05 | 175;286 | 2.16 | 158;162 | 2.34 |
| 72;280 | 9.37 | 222;336 | 2.25 | 251;356 | 2.85 | 7;304 | 6.90 | 134;280 | 2.16 | 70;158 | 2.34 |
| 135;280 | 9.37 | 65;298 | 2.25 | 166;262 | 2.85 | 277;304 | 6.90 | 175;280 | 2.16 | 101;158 | 2.34 |
| 108;280 | 9.37 | 161;360 | 2.25 | 211;251 | 2.85 | 143;305 | 6.90 | 91;280 | 2.16 | 84;128 | 2.34 |
| 203;281 | 9.37 | 65;343 | 2.25 | 218;251 | 2.85 | 118;304 | 6.90 | 187;280 | 2.16 | 158;214 | 2.34 |
| 265;280 | 9.37 | 163;239 | 2.25 | 166;207 | 2.85 | 155;305 | 6.90 | 31;209 | 2.16 | 153;333 | 2.34 |
| 281;298 | 9.37 | 65;342 | 2.25 | 166;210 | 2.85 | 76;304 | 6.90 | 277;282 | 2.16 | 158;219 | 2.34 |
| 280;282 | 9.37 | 222;250 | 2.25 | 227;251 | 2.85 | 152;304 | 6.90 | 90;282 | 2.16 | 69;132 | 2.34 |
| 196;280 | 9.37 | 171;337 | 2.25 | 216;251 | 2.85 | 1;304 | 6.90 | 90;280 | 2.16 | 27;158 | 2.34 |
| 215;280 | 9.37 | 65;338 | 2.25 | 64;167 | 2.85 | 131;305 | 6.90 | 185;282 | 2.16 | 173;240 | 2.34 |
| 280;300 | 9.37 | 201;250 | 2.25 | 214;251 | 2.85 | 275;305 | 6.90 | 185;280 | 2.16 | 158;336 | 2.34 |
| 174;280 | 9.37 | 65;337 | 2.25 | 210;251 | 2.85 | 130;304 | 6.90 | 49;280 | 2.16 | 158;164 | 2.34 |
| 154;280 | 9.37 | 65;336 | 2.25 | 89;251 | 2.85 | 137;304 | 6.90 | 49;282 | 2.16 | 158;171 | 2.34 |
| 254;280 | 9.37 | 195;336 | 2.25 | 154;251 | 2.85 | 61;304 | 6.90 | 25;280 | 2.16 | 106;169 | 2.34 |
| 280;325 | 9.37 | 171;348 | 2.25 | 60;251 | 2.85 | 58;304 | 6.90 | 342;352 | 2.16 | 158;174 | 2.34 |
| 214;280 | 9.37 | 52;337 | 2.25 | 251;363 | 2.85 | 24;304 | 6.90 | 89;280 | 2.16 | 87;158 | 2.34 |
| 206;280 | 9.37 | 171;360 | 2.25 | 251;362 | 2.85 | 305;356 | 6.90 | 176;280 | 2.16 | 139;321 | 2.34 |
| 153;281 | 9.37 | 166;250 | 2.25 | 251;360 | 2.85 | 305;334 | 6.90 | 92;280 | 2.16 | 158;298 | 2.34 |
| 153;280 | 9.37 | 199;362 | 2.25 | 251;359 | 2.85 | 305;320 | 6.90 | 280;324 | 2.16 | 158;350 | 2.34 |
| 173;280 | 9.37 | 149;250 | 2.25 | 230;251 | 2.85 | 72;304 | 6.90 | 98;342 | 2.16 | 20;158 | 2.34 |
| 18;280 | 9.37 | 294;316 | 2.25 | 207;251 | 2.85 | 305;316 | 6.90 | 94;296 | 2.16 | 139;330 | 2.34 |
| 129;280 | 9.37 | 82;250 | 2.25 | 207;254 | 2.85 | 305;359 | 6.90 | 94;285 | 2.16 | 146;165 | 2.34 |
| 280;321 | 9.37 | 158;165 | 2.25 | 156;246 | 2.85 | 85;304 | 6.90 | 94;280 | 2.16 | 139;333 | 2.34 |
| 280;291 | 9.37 | 294;336 | 2.25 | 209;251 | 2.85 | 92;304 | 6.90 | 280;363 | 2.16 | 67;158 | 2.34 |
| 12;280 | 9.37 | 294;337 | 2.25 | 167;195 | 2.85 | 94;304 | 6.90 | 280;361 | 2.16 | 158;203 | 2.34 |
| 2;280 | 9.37 | 65;229 | 2.25 | 208;254 | 2.85 | 304;364 | 6.90 | 280;348 | 2.16 | 152;164 | 2.34 |
| 156;280 | 9.37 | 146;193 | 2.25 | 208;251 | 2.85 | 291;304 | 6.90 | 189;280 | 2.16 | 165;167 | 2.34 |
| 11;280 | 9.37 | 169;203 | 2.25 | 88;251 | 2.85 | 300;305 | 6.90 | 280;342 | 2.16 | 19;171 | 2.34 |
| 9;280 | 9.37 | 65;192 | 2.25 | 88;254 | 2.85 | 102;304 | 6.90 | 280;341 | 2.16 | 86;158 | 2.34 |
| 280;309 | 9.37 | 250;316 | 2.25 | 143;251 | 2.85 | 104;304 | 6.90 | 280;338 | 2.16 | 240;324 | 2.34 |
| 183;280 | 9.37 | 250;343 | 2.25 | 186;254 | 2.85 | 35;304 | 6.90 | 280;336 | 2.16 | 164;193 | 2.34 |
| 176;280 | 9.37 | 14;250 | 2.25 | 186;251 | 2.85 | 294;305 | 6.90 | 280;335 | 2.16 | 169;240 | 2.34 |
| 179;280 | 9.37 | 107;193 | 2.25 | 139;251 | 2.85 | 294;304 | 6.90 | 280;323 | 2.16 | 158;240 | 2.34 |
| 94;280 | 9.37 | 250;341 | 2.25 | 171;251 | 2.85 | 108;304 | 6.90 | 280;320 | 2.16 | 86;164 | 2.34 |
| 192;280 | 9.37 | 309;338 | 2.25 | 184;220 | 2.85 | 304;362 | 6.90 | 280;304 | 2.16 | 157;294 | 2.34 |
| 208;280 | 9.37 | 89;155 | 2.25 | 243;251 | 2.85 | 98;304 | 6.90 | 280;298 | 2.16 | 164;181 | 2.34 |
| 166;280 | 9.37 | 250;340 | 2.25 | 169;251 | 2.85 | 304;360 | 6.90 | 257;296 | 2.16 | 161;190 | 2.34 |
| 177;280 | 9.37 | 165;350 | 2.25 | 169;351 | 2.85 | 304;359 | 6.90 | 93;280 | 2.16 | 68;158 | 2.34 |
| 85;280 | 9.37 | 309;342 | 2.25 | 144;251 | 2.85 | 304;356 | 6.90 | 23;233 | 2.16 | 158;193 | 2.34 |
| 217;280 | 9.37 | 250;338 | 2.25 | 170;251 | 2.85 | 304;352 | 6.90 | 188;280 | 2.16 | 164;190 | 2.34 |
| 86;280 | 9.37 | 250;337 | 2.25 | 113;251 | 2.85 | 304;351 | 6.90 | 173;280 | 2.16 | 158;229 | 2.34 |
| 157;280 | 9.37 | 250;336 | 2.25 | 183;254 | 2.85 | 304;341 | 6.90 | 143;280 | 2.16 | 158;204 | 2.34 |
| 207;280 | 9.37 | 250;335 | 2.25 | 183;251 | 2.85 | 304;339 | 6.90 | 155;280 | 2.16 | 158;195 | 2.34 |
| 151;281 | 9.37 | 250;334 | 2.25 | 9;251 | 2.85 | 304;331 | 6.90 | 209;280 | 2.16 | 158;247 | 2.34 |
| 169;280 | 9.37 | 250;331 | 2.25 | 173;254 | 2.85 | 304;330 | 6.90 | 149;306 | 2.16 | 158;194 | 2.34 |
| 202;331 | 9.37 | 250;330 | 2.25 | 177;251 | 2.85 | 304;324 | 6.90 | 149;290 | 2.16 | 19;169 | 2.34 |
| 222;280 | 9.37 | 250;325 | 2.25 | 130;251 | 2.85 | 304;316 | 6.90 | 149;280 | 2.16 | 106;321 | 2.34 |
| 155;280 | 9.37 | 250;320 | 2.25 | 173;251 | 2.85 | 304;315 | 6.90 | 208;280 | 2.16 | 158;322 | 2.34 |
| 280;362 | 9.37 | 196;250 | 2.25 | 32;251 | 2.85 | 107;304 | 6.90 | 11;352 | 2.16 | 158;254 | 2.34 |
| 97;280 | 9.37 | 89;192 | 2.25 | 127;251 | 2.85 | 213;304 | 6.90 | 207;280 | 2.16 | 158;208 | 2.34 |
| 216;280 | 9.37 | 250;315 | 2.25 | 181;250 | 2.85 | 176;304 | 6.90 | 12;106 | 2.16 | 152;158 | 2.34 |
| 213;280 | 9.37 | 250;350 | 2.25 | 171;351 | 2.85 | 262;304 | 6.90 | 112;280 | 2.16 | 158;230 | 2.34 |
| 167;331 | 9.37 | 195;358 | 2.25 | 181;254 | 2.85 | 49;304 | 6.90 | 40;280 | 2.16 | 164;205 | 2.34 |
| 280;361 | 9.37 | 173;358 | 2.25 | 181;251 | 2.85 | 215;304 | 6.90 | 124;280 | 2.16 | 158;188 | 2.34 |
| 186;280 | 9.37 | 163;193 | 2.25 | 32;351 | 2.85 | 215;305 | 6.90 | 124;282 | 2.16 | 153;158 | 2.34 |
| 280;359 | 9.37 | 65;222 | 2.25 | 30;251 | 2.85 | 186;304 | 6.90 | 124;285 | 2.16 | 158;267 | 2.34 |
| 149;281 | 9.37 | 163;199 | 2.25 | 135;251 | 2.85 | 201;305 | 6.90 | 315;364 | 2.16 | 48;158 | 2.34 |
| 151;280 | 9.37 | 107;296 | 2.25 | 244;251 | 2.85 | 217;304 | 6.90 | 209;342 | 2.16 | 62;169 | 2.34 |
| 89;280 | 9.37 | 149;362 | 2.25 | 9;252 | 2.85 | 172;304 | 6.90 | 7;329 | 2.16 | 158;201 | 2.34 |
| 18;281 | 8.05 | 165;361 | 2.25 | 251;275 | 2.85 | 43;304 | 6.90 | 212;352 | 2.16 | 165;169 | 2.34 |
| 280;343 | 8.05 | 75;250 | 2.25 | 251;283 | 2.85 | 212;304 | 6.90 | 119;285 | 2.16 | 158;190 | 2.34 |
| 128;280 | 8.05 | 165;360 | 2.25 | 251;282 | 2.85 | 177;304 | 6.90 | 152;299 | 2.16 | 147;158 | 2.34 |
| 296;331 | 8.05 | 107;292 | 2.25 | 251;279 | 2.85 | 50;304 | 6.90 | 212;280 | 2.16 | 24;158 | 2.34 |

| | | Extravillous | | | | | | | | Hematopoietic stem | |
|---|---|---|---|---|---|---|---|---|---|---|---|
| Excitatory neurons | | trophoblasts | | Ganglion cells | | Goblet cells | | Granule neurons | | cells | |
| Clusters | Score | Clusters | Score | Clusters | Score | Clusters | Score | Clusters | Score | Clusters | Score |
| 66;257 | 3.30 | 245;274 | 4.35 | 79;312 | 3.57 | 60;87 | 3.45 | 74;355 | 4.75 | 41;169 | 7.41 |
| 31;69 | 3.30 | 68;274 | 3.12 | 142;312 | 3.57 | 60;83 | 3.45 | 66;324 | 3.60 | 162;191 | 6.03 |
| 213;226 | 3.30 | 89;274 | 3.12 | 312;326 | 3.57 | 60;161 | 3.45 | 73;192 | 3.60 | 53;162 | 6.03 |
| 85;226 | 3.30 | 214;274 | 3.12 | 36;312 | 2.66 | 322;353 | 3.45 | 68;266 | 3.60 | 169;191 | 6.03 |
| 146;266 | 3.30 | 213;274 | 3.12 | 32;311 | 2.66 | 13;60 | 3.45 | 66;291 | 3.60 | 173;220 | 6.03 |
| 226;330 | 3.30 | 201;241 | 3.12 | 61;312 | 2.66 | 43;322 | 3.45 | 73;152 | 3.60 | 31;325 | 6.03 |
| 182;183 | 3.30 | 206;274 | 3.12 | 113;313 | 2.66 | 43;316 | 3.45 | 73;153 | 3.60 | 158;199 | 6.03 |
| 217;226 | 3.30 | 201;242 | 3.12 | 98;312 | 2.66 | 60;266 | 3.45 | 73;174 | 3.60 | 41;210 | 6.03 |
| 212;226 | 3.30 | 234;274 | 3.12 | 22;313 | 2.66 | 60;181 | 2.45 | 73;178 | 3.60 | 72;194 | 6.03 |
| 30;296 | 3.30 | 129;283 | 3.12 | 69;293 | 2.66 | 60;89 | 2.45 | 11;74 | 3.60 | 53;163 | 6.03 |
| 184;226 | 3.30 | 238;360 | 3.12 | 72;312 | 2.66 | 115;251 | 2.45 | 74;209 | 3.60 | 41;173 | 6.03 |
| 30;292 | 3.30 | 172;274 | 3.12 | 250;313 | 2.66 | 60;182 | 2.45 | 73;240 | 3.60 | 31;169 | 6.03 |
| 226;291 | 3.30 | 201;202 | 3.12 | 101;312 | 2.66 | 200;248 | 2.45 | 65;255 | 3.60 | 41;171 | 6.03 |
| 59;243 | 3.30 | 82;282 | 3.12 | 164;256 | 2.66 | 60;91 | 2.45 | 73;239 | 3.60 | 41;170 | 6.03 |
| 66;243 | 3.30 | 201;238 | 3.12 | 239;314 | 2.66 | 60;183 | 2.45 | 30:68 | 3.60 | 41;167 | 6.03 |
| 95;242 | 3.30 | 204;274 | 2.04 | 30;267 | 2.66 | 83;300 | 2.45 | 66;258 | 3.60 | 41;162 | 6.03 |
| 180;226 | 3.30 | 32;272 | 2.04 | 103;312 | 2.66 | 60;318 | 2.45 | 73;210 | 3.60 | 31;161 | 6.03 |
| 66;197 | 3.30 | 93;336 | 2.04 | 35;312 | 2.66 | 60;320 | 2.45 | 30;73 | 3.60 | 31;69 | 6.03 |
| 129;226 | 3.30 | 32;189 | 2.04 | 253;293 | 2.66 | 61;302 | 2.45 | 111;134 | 2.56 | 116;199 | 6.03 |
| 226;233 | 3.30 | 139;356 | 2.04 | 179;294 | 2.66 | 60;322 | 2.45 | 74;336 | 2.56 | 53;191 | 4.75 |
| 65;243 | 3.30 | 245;282 | 2.04 | 131;311 | 2.66 | 60;323 | 2.45 | 74;359 | 2.56 | 43;161 | 4.75 |
| 59;226 | 3.30 | 32;274 | 2.04 | 179;256 | 2.66 | 60;225 | 2.45 | 68;144 | 2.56 | 43;162 | 4.75 |
| 71;226 | 3.30 | 94;282 | 2.04 | 220;236 | 2.66 | 48;322 | 2.45 | 74;363 | 2.56 | 31;210 | 4.75 |
| 226;281 | 3.30 | 175;214 | 2.04 | 251;313 | 2.66 | 153;322 | 2.45 | 50;87 | 2.56 | 158;239 | 4.75 |
| 226;290 | 3.30 | 18;282 | 2.04 | 94;312 | 2.66 | 60;131 | 2.45 | 68;136 | 2.56 | 191;325 | 4.75 |
| 226;353 | 3.30 | 89;283 | 2.04 | 95;312 | 2.66 | 60;363 | 2.45 | 74;360 | 2.56 | 53;199 | 4.75 |
| 182;213 | 3.30 | 79;201 | 2.04 | 121;313 | 2.66 | 60;138 | 2.45 | 50;71 | 2.56 | 31;198 | 4.75 |
| 19;243 | 3.30 | 245;357 | 2.04 | 77;312 | 2.66 | 78;113 | 2.45 | 50;69 | 2.56 | 43;348 | 4.75 |
| 66;355 | 3.30 | 89;238 | 2.04 | 183;312 | 2.66 | 87;250 | 2.45 | 20;30 | 2.56 | 41;325 | 4.75 |
| 91;217 | 3.30 | 89;242 | 2.04 | 58;293 | 2.66 | 60;179 | 2.45 | 68;121 | 2.56 | 41;324 | 4.75 |
| 175;226 | 3.30 | 89;252 | 2.04 | 83;313 | 2.66 | 135;251 | 2.45 | 74;347 | 2.56 | 117;239 | 4.75 |
| 96;226 | 3.30 | 279;338 | 2.04 | 73;312 | 2.66 | 215;322 | 2.45 | 68;103 | 2.56 | 158;193 | 4.75 |
| 96;243 | 3.30 | 94;201 | 2.04 | 122;308 | 2.66 | 60;231 | 2.45 | 68;100 | 2.56 | 158;194 | 4.75 |
| 69;243 | 3.30 | 89;272 | 2.04 | 122;312 | 2.66 | 60;82 | 2.45 | 20;69 | 2.56 | 158;198 | 4.75 |
| 226;359 | 3.30 | 93;363 | 2.04 | 30;312 | 2.66 | 214;322 | 2.45 | 115;336 | 2.56 | 158;202 | 4.75 |
| 226;360 | 3.30 | 89;229 | 2.04 | 30;313 | 2.66 | 167;322 | 2.45 | 146;310 | 2.56 | 116;356 | 4.75 |
| 176;226 | 3.30 | 89;237 | 2.04 | 30;314 | 2.66 | 183;212 | 2.45 | 20;194 | 2.56 | 41;198 | 4.75 |
| 19;309 | 3.30 | 139;330 | 2.04 | 9;256 | 2.66 | 19:60 | 2.45 | 66;77 | 2.56 | 41;199 | 4.75 |
| 66;354 | 3.30 | 245;296 | 2.04 | 118;312 | 2.66 | 60;315 | 2.45 | 74;237 | 2.56 | 31;162 | 4.75 |
| 167;247 | 2.16 | 139;282 | 2.04 | 180;312 | 2.66 | 60;86 | 2.45 | 66;83 | 2.56 | 117;199 | 4.75 |
| 170;290 | 2.16 | 274;336 | 2.04 | 214;217 | 2.66 | 152;322 | 2.45 | 146;196 | 2.56 | 53;68 | 4.75 |
| 12;65 | 2.16 | 238;338 | 2.04 | 313;321 | 2.66 | 67;322 | 2.45 | 111;253 | 2.56 | 43;193 | 4.75 |
| 128;243 | 2.16 | 274;324 | 2.04 | 313;327 | 2.66 | 80;83 | 2.45 | 65;336 | 2.56 | 220;325 | 4.75 |
| 167;226 | 2.16 | 274;320 | 2.04 | 127;312 | 2.66 | 60;222 | 2.45 | 42;68 | 2.56 | 31;53 | 4.75 |
| 290;307 | 2.16 | 142;254 | 2.04 | 184;312 | 2.66 | 60;211 | 2.45 | 111;150 | 2.56 | 43;202 | 4.75 |
| 210;226 | 2.16 | 238;330 | 2.04 | 209;351 | 2.66 | 60;115 | 2.45 | 111;164 | 2.56 | 43;216 | 4.75 |
| 222;226 | 2.16 | 274;363 | 2.04 | 56;293 | 2.66 | 87;148 | 2.45 | 111;166 | 2.56 | 41;69 | 4.75 |
| 129;182 | 2.16 | 274;360 | 2.04 | 241;312 | 2.66 | 87;138 | 2.45 | 111;167 | 2.56 | 158;297 | 4.75 |
| 12;243 | 2.16 | 274;356 | 2.04 | 77;209 | 2.66 | 60;354 | 2.45 | 111;169 | 2.56 | 43;169 | 4.75 |
| 169;226 | 2.16 | 238;320 | 2.04 | 112;312 | 2.66 | 60;198 | 2.45 | 111;171 | 2.56 | 41;194 | 4.75 |
| 222;290 | 2.16 | 64;282 | 2.04 | 30;211 | 2.66 | 20;80 | 2.45 | 69;347 | 2.56 | 41;193 | 4.75 |
| 170;247 | 2.16 | 274;348 | 2.04 | 26;244 | 2.66 | 60;200 | 2.45 | 69;355 | 2.56 | 53;169 | 4.75 |
| 170;226 | 2.16 | 92;358 | 2.04 | 137;312 | 2.66 | 60;108 | 2.45 | 255;262 | 2.56 | 31;173 | 4.75 |
| 125;244 | 2.16 | 272;360 | 2.04 | 254;315 | 2.66 | 60;353 | 2.45 | 69;363 | 2.56 | 31;170 | 4.75 |
| 72;226 | 2.16 | 215;274 | 2.04 | 307;312 | 2.66 | 60;210 | 2.45 | 69;319 | 2.56 | 53;161 | 4.75 |
| 290;354 | 2.16 | 215;272 | 2.04 | 85;312 | 2.66 | 60;357 | 2.45 | 173;355 | 2.56 | 116;194 | 4.75 |
| 290;360 | 2.16 | 186;282 | 2.04 | 187;312 | 2.66 | 60;202 | 2.45 | 97;111 | 2.56 | 53;158 | 4.75 |
| 168;226 | 2.16 | 92;296 | 2.04 | 306;312 | 2.66 | 60;358 | 2.45 | 18;73 | 2.56 | 41;161 | 4.75 |
| 290;355 | 2.16 | 272;356 | 2.04 | 254;293 | 2.66 | 43;193 | 2.45 | 68;238 | 2.56 | 116;297 | 4.75 |
| 210;290 | 2.16 | 92;274 | 2.04 | 63;312 | 2.66 | 60;208 | 2.45 | 229;255 | 2.56 | 129;193 | 4.75 |
| 70;226 | 2.16 | 272;330 | 2.04 | 212;313 | 1.86 | 43;197 | 2.45 | 64;237 | 2.56 | 198;325 | 4.75 |
| 218;356 | 2.16 | 238;363 | 2.04 | 267;293 | 1.86 | 148;251 | 2.45 | 50;220 | 2.56 | 53;348 | 4.75 |
| 70;91 | 2.16 | 274;292 | 2.04 | 120;312 | 1.86 | 60;351 | 2.45 | 50;214 | 2.56 | 53;356 | 4.75 |
| 164;226 | 2.16 | 238;356 | 2.04 | 167;306 | 1.86 | 60;77 | 2.45 | 50;212 | 2.56 | 116;296 | 4.75 |
| 69;361 | 2.16 | 35;282 | 2.04 | 17;313 | 1.86 | 60;340 | 2.45 | 50;194 | 2.56 | 129;194 | 4.75 |
| 218;290 | 2.16 | 93;146 | 2.04 | 112;293 | 1.86 | 60;185 | 2.45 | 50;179 | 2.56 | 42;198 | 4.75 |
| 69;311 | 2.16 | 93;213 | 2.04 | 187;293 | 1.86 | 87;113 | 2.45 | 50;173 | 2.56 | 116;244 | 4.75 |
| 211;309 | 2.16 | 93;215 | 2.04 | 167;267 | 1.86 | 60;325 | 2.45 | 11;69 | 2.56 | 194;325 | 3.60 |
| 69;297 | 2.16 | 93;214 | 2.04 | 154;312 | 1.86 | 60;220 | 2.45 | 50;163 | 2.56 | 116;193 | 3.60 |
| 69;294 | 2.16 | 32;345 | 2.04 | 113;239 | 1.86 | 60;101 | 2.45 | 50;155 | 2.56 | 31;167 | 3.60 |
| 69;290 | 2.16 | 93;206 | 2.04 | 236;256 | 1.86 | 60;188 | 2.45 | 50;153 | 2.56 | 192;325 | 3.60 |
| 134;226 | 2.16 | 93;201 | 2.04 | 7;312 | 1.86 | 60;334 | 2.45 | 50;145 | 2.56 | 116;170 | 3.60 |
| 69;266 | 2.16 | 36;201 | 2.04 | 153;313 | 1.86 | 185;322 | 2.45 | 50;338 | 2.56 | 63;170 | 3.60 |
| 69;258 | 2.16 | 245;272 | 2.04 | 121;147 | 1.86 | 60;346 | 2.45 | 34;66 | 2.56 | 70;198 | 3.60 |
| 69;257 | 2.16 | 114;356 | 2.04 | 17;312 | 1.86 | 60;105 | 2.45 | 50;256 | 2.56 | 188;191 | 3.60 |
| 69;256 | 2.16 | 32;296 | 2.04 | 69;101 | 1.86 | 20;60 | 2.45 | 50;244 | 2.56 | 176;194 | 3.60 |
| 69;249 | 2.16 | 89;354 | 2.04 | 112;264 | 1.86 | 248;302 | 2.45 | 11;71 | 2.56 | 70;194 | 3.60 |
| 219;243 | 2.16 | 254;283 | 2.04 | 195;313 | 1.86 | 133;200 | 2.45 | 111;256 | 2.56 | 72;193 | 3.60 |
| 11;247 | 2.16 | 238;282 | 2.04 | 195;312 | 1.86 | 222;322 | 2.45 | 66;266 | 2.56 | 116;161 | 3.60 |
| 11;226 | 2.16 | 93;161 | 2.04 | 231;312 | 1.86 | 60;214 | 2.45 | 193;310 | 2.56 | 72;198 | 3.60 |
| 71;247 | 2.16 | 93;173 | 2.04 | 167;312 | 1.86 | 60;119 | 2.45 | 9;66 | 2.56 | 116;202 | 3.60 |
| 295;303 | 2.16 | 93;172 | 2.04 | 154;313 | 1.86 | 9;60 | 2.45 | 73;237 | 2.56 | 69;158 | 3.60 |
| 11;91 | 2.16 | 93;170 | 2.04 | 119;313 | 1.86 | 60;206 | 2.45 | 73;226 | 2.56 | 70;193 | 3.60 |
| 130;226 | 2.16 | 201;297 | 2.04 | 213;312 | 1.86 | 310;322 | 2.45 | 73;213 | 2.56 | 79;169 | 3.60 |
| 296;303 | 2.16 | 131;274 | 2.04 | 213;313 | 1.86 | 60;164 | 2.45 | 73;208 | 2.56 | 78;161 | 3.60 |
| 71;91 | 2.16 | 201;315 | 2.04 | 155;312 | 1.86 | 162;322 | 2.45 | 73;266 | 2.56 | 158;296 | 3.60 |
| 296;348 | 2.16 | 126;201 | 2.04 | 87;313 | 1.86 | 156;322 | 2.45 | 115;360 | 2.56 | 116;239 | 3.60 |
| 166;226 | 2.16 | 97;252 | 2.04 | 225;313 | 1.86 | 60;153 | 2.45 | 73;202 | 2.56 | 41;53 | 3.60 |
| 211;226 | 2.16 | 87;201 | 2.04 | 87;312 | 1.86 | 80;225 | 2.45 | 66;294 | 2.56 | 78;194 | 3.60 |
| 73;216 | 2.16 | 50;274 | 2.04 | 214;229 | 1.86 | 223;322 | 2.45 | 73;185 | 2.56 | 69;78 | 3.60 |
| 73;226 | 2.16 | 97;280 | 2.04 | 112;313 | 1.86 | 60;174 | 2.45 | 71;355 | 2.56 | 129;169 | 3.60 |
| 226;321 | 2.16 | 97;283 | 2.04 | 225;312 | 1.86 | 60;265 | 2.45 | 66;296 | 2.56 | 43;214 | 3.60 |
| 226;356 | 2.16 | 201;291 | 2.04 | 69;226 | 1.86 | 60;146 | 2.45 | 144;229 | 2.56 | 177;198 | 3.60 |
| 226;355 | 2.16 | 73;274 | 2.04 | 71;236 | 1.86 | 60;166 | 2.45 | 66;209 | 2.56 | 117;169 | 3.60 |
| 226;354 | 2.16 | 201;272 | 2.04 | 31;293 | 1.86 | 60;71 | 2.45 | 66;221 | 2.56 | 78;162 | 3.60 |
| 226;352 | 2.16 | 73;282 | 2.04 | 98;244 | 1.86 | 60;291 | 2.45 | 182;214 | 2.56 | 187;191 | 3.60 |
| 226;350 | 2.16 | 73;283 | 2.04 | 119;312 | 1.86 | 78;251 | 2.45 | 182;212 | 2.56 | 119;199 | 3.60 |
| 226;348 | 2.16 | 201;274 | 2.04 | 167;256 | 1.86 | 60;167 | 2.45 | 182;206 | 2.56 | 69;235 | 3.60 |
| 226;346 | 2.16 | 38;337 | 2.04 | 194;312 | 1.86 | 60;152 | 2.45 | 182;196 | 2.56 | 161;242 | 3.60 |
| 226;343 | 2.16 | 201;290 | 2.04 | 80;312 | 1.86 | 60;168 | 2.45 | 182;194 | 2.56 | 117;170 | 3.60 |

| | | | | IGFBP1_DKK1 positive | | | | | | Intestinal epithelial | |
|---|---|---|---|---|---|---|---|---|---|---|---|
| Hepatoblasts | | Horizontal cells | | cells | | Inhibitory interneurons | | Inhibitory neurons | | cells | |
| Clusters | Score | Clusters | Score | Clusters | Score | Clusters | Score | Clusters | Score | Clusters | Score |
| 122;334 | 5.19 | 57;243 | 6.12 | 272;318 | 3.96 | 166;234 | 4.80 | 52;190 | 5.64 | 14;101 | 6.00 |
| 145;214 | 3.93 | 248;256 | 5.06 | 8;318 | 3.96 | 4;249 | 4.80 | 26;262 | 4.78 | 101;107 | 6.00 |
| 40;105 | 3.93 | 59;256 | 5.06 | 318;342 | 3.96 | 4;168 | 4.80 | 136;255 | 4.78 | 145;216 | 4.86 |
| 97;101 | 3.93 | 79;256 | 5.06 | 278;318 | 3.07 | 166;238 | 4.80 | 52;64 | 4.78 | 2;105 | 4.86 |
| 145;265 | 3.93 | 58;293 | 5.06 | 315;326 | 3.07 | 195;238 | 3.86 | 26;207 | 4.78 | 12;101 | 4.86 |
| 100;258 | 3.93 | 73;247 | 4.07 | 272;315 | 3.07 | 234;249 | 3.86 | 95;256 | 4.78 | 101;135 | 4.86 |
| 122;258 | 3.93 | 56;293 | 4.07 | 316;360 | 3.07 | 202;247 | 3.86 | 98;227 | 4.78 | 4;101 | 4.86 |
| 101;134 | 3.93 | 142;256 | 4.07 | 19;315 | 3.07 | 13;241 | 3.86 | 136;253 | 4.78 | 101;131 | 4.86 |
| 101;311 | 3.93 | 58;288 | 4.07 | 91;360 | 3.07 | 8;234 | 3.86 | 176;249 | 3.97 | 101;127 | 4.86 |
| 145;350 | 3.93 | 233;256 | 4.07 | 20;280 | 3.07 | 18;228 | 3.86 | 42;228 | 3.97 | 145;357 | 4.86 |
| 101;125 | 3.93 | 143;256 | 4.07 | 318;347 | 3.07 | 3;239 | 3.86 | 11;26 | 3.97 | 101;126 | 4.86 |
| 105;257 | 3.93 | 69;256 | 4.07 | 316;326 | 3.07 | 3;246 | 3.86 | 30;136 | 3.97 | 97;101 | 4.86 |
| 61;145 | 3.93 | 72;256 | 4.07 | 154;315 | 3.07 | 3;234 | 3.86 | 38;136 | 3.97 | 101;122 | 4.86 |
| 105;267 | 3.93 | 98;256 | 4.07 | 150;326 | 3.07 | 30;200 | 3.86 | 254;290 | 3.97 | 101;216 | 4.86 |
| 101;258 | 3.93 | 69;243 | 4.07 | 11;315 | 3.07 | 187;197 | 3.86 | 20;26 | 3.97 | 98;101 | 4.86 |
| 145;196 | 3.93 | 157;256 | 4.07 | 204;318 | 2.27 | 18;226 | 3.86 | 26;212 | 3.97 | 101;228 | 4.86 |
| 105;109 | 3.93 | 75;256 | 4.07 | 11;321 | 2.27 | 187;226 | 3.86 | 136;294 | 3.97 | 2;146 | 4.86 |
| 87;145 | 3.93 | 129;256 | 4.07 | 318;334 | 2.27 | 18;241 | 3.86 | 130;254 | 3.97 | 101;150 | 4.86 |
| 87;105 | 3.93 | 187;256 | 4.07 | 325;326 | 2.27 | 37;195 | 3.86 | 158;262 | 3.97 | 16;101 | 4.86 |
| 101;264 | 3.93 | 155;256 | 4.07 | 142;318 | 2.27 | 190;238 | 3.86 | 124;227 | 3.97 | 101;172 | 4.86 |
| 105;308 | 3.93 | 190;256 | 4.07 | 20;318 | 2.27 | 204;234 | 3.86 | 249;254 | 3.97 | 63;101 | 4.86 |
| 122;361 | 3.93 | 53;256 | 4.07 | 280;315 | 2.27 | 241;264 | 3.86 | 12;26 | 3.97 | 101;168 | 4.86 |
| 83;270 | 3.93 | 165;256 | 4.07 | 155;318 | 2.27 | 125;311 | 3.86 | 18;228 | 3.97 | 63;145 | 4.86 |
| 83;258 | 3.93 | 91;256 | 4.07 | 316;347 | 2.27 | 4;37 | 3.86 | 45;254 | 3.97 | 101;357 | 4.86 |
| 48;101 | 3.93 | 42;256 | 4.07 | 153;315 | 2.27 | 92;249 | 3.86 | 26;194 | 3.97 | 62;145 | 4.86 |
| 98;101 | 3.93 | 132;293 | 4.07 | 185;317 | 2.27 | 3;30 | 3.86 | 26;195 | 3.97 | 77;101 | 4.86 |
| 6;105 | 3.93 | 59;247 | 4.07 | 173;318 | 2.27 | 4;234 | 3.86 | 33;254 | 3.97 | 2;61 | 4.86 |
| 101;341 | 3.93 | 58;247 | 4.07 | 153;326 | 2.27 | 11;92 | 3.86 | 41;254 | 3.97 | 58;145 | 4.86 |
| 122;147 | 3.93 | 71;256 | 4.07 | 153;321 | 2.27 | 4;258 | 3.86 | 39;254 | 3.97 | 2;72 | 4.86 |
| 101;239 | 2.81 | 56;243 | 4.07 | 212;282 | 2.27 | 136;179 | 2.99 | 26;171 | 3.97 | 11;101 | 4.86 |
| 101;238 | 2.81 | 67;256 | 4.07 | 118;318 | 2.27 | 92;95 | 2.99 | 26;169 | 3.97 | 99;101 | 4.86 |
| 101;186 | 2.81 | 58;256 | 4.07 | 153;318 | 2.27 | 7;227 | 2.99 | 262;325 | 3.97 | 102;145 | 4.86 |
| 101;185 | 2.81 | 17;256 | 4.07 | 180;318 | 2.27 | 11;236 | 2.99 | 26;136 | 3.97 | 101;232 | 4.86 |
| 101;229 | 2.81 | 121;293 | 4.07 | 66;282 | 2.27 | 13;228 | 2.99 | 21;65 | 3.97 | 10;145 | 4.86 |
| 101;233 | 2.81 | 144;256 | 4.07 | 317;339 | 2.27 | 7;241 | 2.99 | 5;350 | 3.22 | 76;101 | 4.86 |
| 101;234 | 2.81 | 19;256 | 4.07 | 269;318 | 2.27 | 7;244 | 2.99 | 30;76 | 3.22 | 101;294 | 4.86 |
| 64;101 | 2.81 | 19;293 | 3.16 | 25;356 | 2.27 | 177;226 | 2.99 | 30;85 | 3.22 | 101;111 | 4.86 |
| 101;206 | 2.81 | 256;352 | 3.16 | 191;326 | 2.27 | 187;244 | 2.99 | 136;168 | 3.22 | 122;145 | 4.86 |
| 5;101 | 2.81 | 59;243 | 3.16 | 125;318 | 2.27 | 3;244 | 2.99 | 230;266 | 3.22 | 101;270 | 4.86 |
| 101;205 | 2.81 | 228;243 | 3.16 | 278;316 | 2.27 | 201;241 | 2.99 | 52;68 | 3.22 | 101;102 | 4.86 |
| 101;240 | 2.81 | 222;256 | 3.16 | 5;318 | 2.27 | 7;176 | 2.99 | 41;166 | 3.22 | 2;214 | 4.86 |
| 101;184 | 2.81 | 212;256 | 3.16 | 52;318 | 2.27 | 179;241 | 2.99 | 50;227 | 3.22 | 58;101 | 4.86 |
| 101;183 | 2.81 | 59;288 | 3.16 | 240;318 | 2.27 | 92;124 | 2.99 | 57;254 | 3.22 | 2;171 | 4.86 |
| 101;182 | 2.81 | 171;256 | 3.16 | 196;317 | 2.27 | 11;226 | 2.99 | 181;227 | 3.22 | 101;106 | 4.86 |
| 101;210 | 2.81 | 253;293 | 3.16 | 342;347 | 2.27 | 13;238 | 2.99 | 181;262 | 3.22 | 2;169 | 4.86 |
| 101;181 | 2.81 | 134;247 | 3.16 | 318;320 | 2.27 | 239;251 | 2.99 | 146;294 | 3.22 | 145;209 | 4.86 |
| 101;203 | 2.81 | 212;293 | 3.16 | 274;282 | 2.27 | 234;264 | 2.99 | 158;266 | 3.22 | 10;101 | 4.86 |
| 101;180 | 2.81 | 72;293 | 3.16 | 16;194 | 2.27 | 13;239 | 2.99 | 158;254 | 3.22 | 103;146 | 4.86 |
| 101;179 | 2.81 | 24;256 | 3.16 | 202;318 | 2.27 | 37;247 | 2.99 | 50;266 | 3.22 | 86;101 | 4.86 |
| 101;178 | 2.81 | 256;364 | 3.16 | 239;318 | 2.27 | 202;226 | 2.99 | 30;43 | 3.22 | 101;180 | 3.81 |
| 101;236 | 2.81 | 188;256 | 3.16 | 42;315 | 2.27 | 202;234 | 2.99 | 52;59 | 3.22 | 101;181 | 3.81 |
| 101;237 | 2.81 | 188;293 | 3.16 | 208;280 | 2.27 | 20;244 | 2.99 | 30;166 | 3.22 | 145;350 | 3.81 |
| 101;202 | 2.81 | 203;256 | 3.16 | 86;318 | 2.27 | 202;244 | 2.99 | 30;227 | 3.22 | 101;196 | 3.81 |
| 101;177 | 2.81 | 185;293 | 3.16 | 133;282 | 2.27 | 202;246 | 2.99 | 21;252 | 3.22 | 145;351 | 3.81 |
| 101;176 | 2.81 | 121;256 | 3.16 | 318;326 | 2.27 | 238;262 | 2.99 | 44;227 | 3.22 | 101;296 | 3.81 |
| 101;204 | 2.81 | 71;293 | 3.16 | 119;318 | 2.27 | 215;234 | 2.99 | 30;195 | 3.22 | 145;352 | 3.81 |
| 101;197 | 2.81 | 238;243 | 3.16 | 222;318 | 2.27 | 238;247 | 2.99 | 41;227 | 3.22 | 145;230 | 3.81 |
| 101;198 | 2.81 | 243;293 | 3.16 | 222;315 | 2.27 | 187;246 | 2.99 | 30;205 | 3.22 | 145;234 | 3.81 |
| 101;228 | 2.81 | 187;297 | 3.16 | 91;347 | 2.27 | 234;309 | 2.99 | 41;228 | 3.22 | 145;353 | 3.81 |
| 101;196 | 2.81 | 160;293 | 3.16 | 319;347 | 2.27 | 127;244 | 2.99 | 35;254 | 3.22 | 122;207 | 3.81 |
| 101;195 | 2.81 | 171;293 | 3.16 | 96;318 | 2.27 | 30;249 | 2.99 | 254;266 | 3.22 | 145;354 | 3.81 |
| 101;211 | 2.81 | 93;293 | 3.16 | 132;318 | 2.27 | 30;250 | 2.99 | 217;266 | 3.22 | 145;217 | 3.81 |
| 101;194 | 2.81 | 256;355 | 3.16 | 318;359 | 2.27 | 238;353 | 2.99 | 254;311 | 3.22 | 145;286 | 3.81 |
| 101;212 | 2.81 | 106;293 | 3.16 | 166;317 | 2.27 | 13;249 | 2.99 | 30;146 | 3.22 | 16;145 | 3.81 |
| 101;213 | 2.81 | 256;359 | 3.16 | 74;326 | 2.27 | 12;234 | 2.99 | 11;57 | 3.22 | 101;278 | 3.81 |
| 101;193 | 2.81 | 242;256 | 3.16 | 280;282 | 2.27 | 91;244 | 2.99 | 30;215 | 3.22 | 101;193 | 3.81 |
| 101;192 | 2.81 | 142;243 | 3.16 | 315;347 | 2.27 | 234;236 | 2.99 | 92;136 | 3.22 | 145;228 | 3.81 |
| 101;209 | 2.81 | 214;217 | 3.16 | 20;272 | 2.27 | 234;238 | 2.99 | 30;266 | 3.22 | 101;177 | 3.81 |
| 101;190 | 2.81 | 60;249 | 3.16 | 224;318 | 2.27 | 238;246 | 2.99 | 30;251 | 3.22 | 58;78 | 3.81 |
| 101;214 | 2.81 | 98;247 | 3.16 | 8;315 | 2.27 | 201;249 | 2.99 | 11;32 | 3.22 | 101;182 | 3.81 |
| 101;215 | 2.81 | 238;293 | 3.16 | 319;326 | 2.27 | 226;264 | 2.99 | 41;195 | 3.22 | 101;178 | 3.81 |
| 101;216 | 2.81 | 112;293 | 3.16 | 126;318 | 2.27 | 226;262 | 2.99 | 212;249 | 3.22 | 145;223 | 3.81 |
| 101;189 | 2.81 | 214;249 | 3.16 | 16;356 | 2.27 | 52;311 | 2.99 | 5;227 | 3.22 | 101;183 | 3.81 |
| 101;217 | 2.81 | 256;348 | 3.16 | 326;342 | 2.27 | 197;246 | 2.99 | 124;247 | 3.22 | 145;222 | 3.81 |
| 101;218 | 2.81 | 178;256 | 3.16 | 200;274 | 2.27 | 131;234 | 2.99 | 26;182 | 3.22 | 101;184 | 3.81 |
| 101;241 | 2.81 | 240;293 | 3.16 | 298;315 | 2.27 | 121;226 | 2.99 | 180;205 | 3.22 | 145;220 | 3.81 |
| 101;188 | 2.81 | 97;256 | 3.16 | 55;317 | 2.27 | 124;236 | 2.99 | 26;186 | 3.22 | 145;237 | 3.81 |
| 101;220 | 2.81 | 20;256 | 3.16 | 194;342 | 2.27 | 226;228 | 2.99 | 26;193 | 3.22 | 101;186 | 3.81 |
| 101;222 | 2.81 | 186;256 | 3.16 | 96;317 | 2.27 | 197;226 | 2.99 | 227;231 | 3.22 | 101;188 | 3.81 |
| 101;208 | 2.81 | 207;256 | 3.16 | 166;315 | 2.27 | 184;226 | 2.99 | 227;242 | 3.22 | 101;291 | 3.81 |
| 101;207 | 2.81 | 50;293 | 3.16 | 109;318 | 2.27 | 241;262 | 2.99 | 227;249 | 3.22 | 101;189 | 3.81 |
| 57;101 | 2.81 | 170;256 | 3.16 | 226;315 | 2.27 | 244;269 | 2.99 | 26;206 | 3.22 | 101;185 | 3.81 |
| 101;223 | 2.81 | 97;293 | 3.16 | 56;282 | 2.27 | 196;234 | 2.99 | 26;210 | 3.22 | 101;240 | 3.81 |
| 101;224 | 2.81 | 256;320 | 3.16 | 272;321 | 2.27 | 244;245 | 2.99 | 137;226 | 3.22 | 101;201 | 3.81 |
| 101;226 | 2.81 | 256;321 | 3.16 | 16;282 | 2.27 | 52;179 | 2.99 | 129;212 | 3.22 | 101;174 | 3.81 |
| 101;187 | 2.81 | 256;323 | 3.16 | 315;324 | 2.27 | 244;264 | 2.99 | 26;214 | 3.22 | 101;262 | 3.81 |
| 101;201 | 2.81 | 125;293 | 3.16 | 318;339 | 2.27 | 244;266 | 2.99 | 85;249 | 3.22 | 101;257 | 3.81 |
| 101;268 | 2.81 | 229;256 | 3.16 | 91;356 | 2.27 | 195;234 | 2.99 | 227;294 | 3.22 | 101;210 | 3.81 |
| 101;242 | 2.81 | 256;292 | 3.16 | 128;272 | 2.27 | 7;52 | 2.99 | 136;354 | 3.22 | 145;270 | 3.81 |
| 101;338 | 2.81 | 256;293 | 3.16 | 316;339 | 2.27 | 3;201 | 2.99 | 226;227 | 3.22 | 145;269 | 3.81 |
| 101;336 | 2.81 | 186;293 | 3.16 | 318;338 | 2.27 | 68;243 | 2.99 | 25;227 | 3.22 | 101;213 | 3.81 |
| 101;334 | 2.81 | 256;289 | 3.16 | 158;318 | 2.27 | 7;79 | 2.99 | 26;59 | 3.22 | 101;214 | 3.81 |
| 101;333 | 2.81 | 103;244 | 3.16 | 72;280 | 2.27 | 68;227 | 2.99 | 15;136 | 3.22 | 101;249 | 3.81 |
| 101;331 | 2.81 | 256;270 | 3.16 | 288;347 | 2.27 | 3;195 | 2.99 | 26;85 | 3.22 | 101;215 | 3.81 |
| 101;330 | 2.81 | 36;247 | 3.16 | 229;315 | 2.27 | 68;197 | 2.99 | 26;95 | 3.22 | 101;217 | 3.81 |
| 101;326 | 2.81 | 256;295 | 3.16 | 12;318 | 2.27 | 227;262 | 2.99 | 26;170 | 3.22 | 101;218 | 3.81 |
| 101;325 | 2.81 | 256;297 | 3.16 | 132;315 | 2.27 | 5;262 | 2.99 | 38;154 | 3.22 | 105;197 | 3.81 |
| 101;324 | 2.81 | 205;256 | 3.16 | 20;274 | 2.27 | 3;236 | 2.99 | 85;325 | 3.22 | 101;225 | 3.81 |
| 101;322 | 2.81 | 206;256 | 3.16 | 278;336 | 2.27 | 28;69 | 2.99 | 5;136 | 3.22 | 101;245 | 3.81 |
| 101;321 | 2.81 | 58;215 | 3.16 | 91;318 | 2.27 | 7;39 | 2.99 | 238;254 | 3.22 | 101;231 | 3.81 |

| Lymphatic endothelial | | | | MUC13_DMBT1 | | | | | | | |
|---|---|---|---|---|---|---|---|---|---|---|---|
| cells | | Lymphoid cells | | positive cells | | Megakaryocytes | | Mesangial cells | | Mesothelial cells | |
| Clusters | Score | Clusters | Score | Clusters | Score | Clusters | Score | Clusters | Score | Clusters | Score |
| 298;358 | 5.73 | 11;326 | 5.34 | 78;150 | 4.07 | 85;147 | 3.76 | 188;278 | 9.88 | 146;353 | 3.93 |
| 176;360 | 5.73 | 96;365 | 4.31 | 101;307 | 4.07 | 157;343 | 3.76 | 172;277 | 9.88 | 55;282 | 3.93 |
| 91;358 | 4.35 | 96;266 | 4.31 | 206;307 | 4.07 | 94;172 | 2.68 | 176;278 | 8.68 | 257;295 | 2.81 |
| 209;316 | 4.35 | 25;96 | 4.31 | 101;157 | 4.07 | 43;252 | 2.68 | 15;277 | 8.68 | 257;296 | 2.81 |
| 193;308 | 4.35 | 96;245 | 4.31 | 101;294 | 4.07 | 67;111 | 2.68 | 276;345 | 8.68 | 261;285 | 2.81 |
| 298;351 | 4.35 | 96;97 | 4.31 | 101;149 | 4.07 | 180;343 | 2.68 | 69;279 | 8.68 | 88;358 | 2.81 |
| 294;316 | 4.35 | 96;211 | 4.31 | 101;201 | 4.07 | 157;238 | 2.68 | 249;357 | 8.68 | 55;356 | 2.81 |
| 40;318 | 4.35 | 6:96 | 4.31 | 14;101 | 4.07 | 94;167 | 2.68 | 174;277 | 8.68 | 188;360 | 2.81 |
| 198;308 | 4.35 | 96;324 | 4.31 | 2;72 | 4.07 | 111;123 | 2.68 | 173;277 | 8.68 | 86;282 | 2.81 |
| 308;358 | 4.35 | 96;347 | 4.31 | 101;161 | 4.07 | 94;166 | 2.68 | 277;325 | 8.68 | 159;299 | 2.81 |
| 304;316 | 4.35 | 133;134 | 4.31 | 101;168 | 4.07 | 50;157 | 2.68 | 90;277 | 8.68 | 277;282 | 2.81 |
| 181;316 | 4.35 | 96;310 | 4.31 | 98;101 | 4.07 | 94;180 | 2.68 | 57;279 | 8.68 | 154;241 | 2.81 |
| 315;359 | 4.35 | 96;355 | 4.31 | 101;357 | 4.07 | 136;364 | 2.68 | 74;277 | 8.68 | 118;299 | 2.81 |
| 298;316 | 4.35 | 96;98 | 4.31 | 2;152 | 4.07 | 34;85 | 2.68 | 276;286 | 8.68 | 152;299 | 2.81 |
| 298;308 | 4.35 | 96;241 | 4.31 | 101;107 | 4.07 | 34;89 | 2.68 | 72;277 | 8.68 | 255;342 | 2.81 |
| 254;298 | 4.35 | 96;270 | 4.31 | 101;146 | 3.16 | 111;148 | 2.68 | 277;278 | 8.68 | 55;330 | 2.81 |
| 212;298 | 4.35 | 171;187 | 3.36 | 69;78 | 3.16 | 22;89 | 2.68 | 76;278 | 8.68 | 146;330 | 2.81 |
| 317;345 | 4.35 | 65;96 | 3.36 | 101;150 | 3.16 | 94;182 | 2.68 | 168;277 | 8.68 | 154;342 | 2.81 |
| 269;298 | 4.35 | 96;209 | 3.36 | 101;148 | 3.16 | 22;355 | 2.68 | 64;277 | 8.68 | 286;299 | 2.81 |
| 318;345 | 3.12 | 96;210 | 3.36 | 101;147 | 3.16 | 94;160 | 2.68 | 279;302 | 8.68 | 280;348 | 2.81 |
| 57;308 | 3.12 | 40;96 | 3.36 | 100;152 | 3.16 | 119;361 | 2.68 | 44;279 | 8.68 | 94;194 | 2.81 |
| 251;300 | 3.12 | 96;205 | 3.36 | 78;211 | 3.16 | 34;150 | 2.68 | 278;353 | 8.68 | 31;338 | 2.81 |
| 317;338 | 3.12 | 96;212 | 3.36 | 101;151 | 3.16 | 185;343 | 2.68 | 9;278 | 8.68 | 106;350 | 2.81 |
| 317;337 | 3.12 | 95;104 | 3.36 | 101;152 | 3.16 | 110;159 | 2.68 | 278;361 | 8.68 | 31;337 | 2.81 |
| 251;302 | 3.12 | 96;213 | 3.36 | 101;340 | 3.16 | 119;295 | 2.68 | 137;278 | 8.68 | 31;336 | 2.81 |
| 298;310 | 3.12 | 42;96 | 3.36 | 101;169 | 3.16 | 136;252 | 2.68 | 276;302 | 8.68 | 106;358 | 2.81 |
| 298;304 | 3.12 | 67;95 | 3.36 | 66;101 | 3.16 | 109;143 | 2.68 | 276;301 | 8.68 | 33;282 | 2.81 |
| 251;317 | 3.12 | 96;325 | 3.36 | 101;167 | 3.16 | 34;154 | 2.68 | 175;278 | 8.68 | 280;340 | 2.81 |
| 199;298 | 3.12 | 96;167 | 3.36 | 101;165 | 3.16 | 94;123 | 2.68 | 73;277 | 8.68 | 96;280 | 2.81 |
| 252;298 | 3.12 | 96;166 | 3.36 | 101;164 | 3.16 | 94;159 | 2.68 | 215;277 | 8.68 | 257;347 | 1.83 |
| 252;305 | 3.12 | 96;169 | 3.36 | 101;163 | 3.16 | 22;252 | 2.68 | 214;277 | 8.68 | 91;282 | 1.83 |
| 317;336 | 3.12 | 96;322 | 3.36 | 101;160 | 3.16 | 34;134 | 2.68 | 16;277 | 8.68 | 21;198 | 1.83 |
| 252;316 | 3.12 | 96;168 | 3.36 | 101;155 | 3.16 | 136;209 | 2.68 | 171;277 | 7.53 | 257;350 | 1.83 |
| 59;305 | 3.12 | 96;208 | 3.36 | 101;154 | 3.16 | 105;136 | 2.68 | 278;286 | 7.53 | 257;351 | 1.83 |
| 325;360 | 3.12 | 92;96 | 3.36 | 101;145 | 3.16 | 136;204 | 2.68 | 58;276 | 7.53 | 152;257 | 1.83 |
| 86;308 | 3.12 | 96;207 | 3.36 | 101;143 | 3.16 | 111;159 | 2.68 | 145;276 | 7.53 | 318;342 | 1.83 |
| 57;358 | 3.12 | 18;96 | 3.36 | 101;117 | 3.16 | 119;360 | 2.68 | 205;277 | 7.53 | 257;356 | 1.83 |
| 57;336 | 3.12 | 96;323 | 3.36 | 101;121 | 3.16 | 22;330 | 2.68 | 179;278 | 7.53 | 257;358 | 1.83 |
| 250;330 | 3.12 | 20;326 | 3.36 | 72;101 | 3.16 | 94;364 | 2.68 | 248;276 | 7.53 | 257;360 | 1.83 |
| 318;358 | 3.12 | 96;206 | 3.36 | 101;119 | 3.16 | 94;363 | 2.68 | 278;291 | 7.53 | 257;363 | 1.83 |
| 89;316 | 3.12 | 162;187 | 3.36 | 101;118 | 3.16 | 67;135 | 2.68 | 164;277 | 7.53 | 152;353 | 1.83 |
| 298;359 | 3.12 | 87;187 | 3.36 | 78;147 | 3.16 | 23;157 | 2.68 | 170;277 | 7.53 | 257;353 | 1.83 |
| 137;316 | 3.12 | 67;96 | 3.36 | 101;111 | 3.16 | 22;143 | 2.68 | 98;277 | 7.53 | 58;282 | 1.83 |
| 298;354 | 3.12 | 37;96 | 3.36 | 103;206 | 3.16 | 150;343 | 2.68 | 278;294 | 7.53 | 286;324 | 1.83 |
| 250;318 | 3.12 | 96;222 | 3.36 | 101;106 | 3.16 | 22;138 | 2.68 | 278;298 | 7.53 | 152;194 | 1.83 |
| 298;352 | 3.12 | 15;96 | 3.36 | 78;105 | 3.16 | 119;351 | 2.68 | 58;278 | 7.53 | 171;257 | 1.83 |
| 250;300 | 3.12 | 19;96 | 3.36 | 219;306 | 3.16 | 22;154 | 2.68 | 273;276 | 7.53 | 257;282 | 1.83 |
| 207;298 | 3.12 | 6;326 | 3.36 | 78;171 | 3.16 | 171;343 | 2.68 | 277;353 | 7.53 | 188;280 | 1.83 |
| 57;318 | 3.12 | 96;121 | 3.36 | 101;135 | 3.16 | 111;321 | 2.68 | 238;277 | 7.53 | 171;280 | 1.83 |
| 203;316 | 3.12 | 96;223 | 3.36 | 152;364 | 3.16 | 119;330 | 2.68 | 39;279 | 7.53 | 151;358 | 1.83 |
| 317;347 | 3.12 | 87;96 | 3.36 | 145;278 | 3.16 | 211;252 | 2.68 | 71;277 | 7.53 | 21;356 | 1.83 |
| 203;298 | 3.12 | 7;95 | 3.36 | 19;101 | 3.16 | 23;175 | 2.68 | 17;276 | 7.53 | 21;360 | 1.83 |
| 298;340 | 3.12 | 96;152 | 3.36 | 78;172 | 3.16 | 22;157 | 2.68 | 150;277 | 7.53 | 194;320 | 1.83 |
| 207;292 | 3.12 | 12;96 | 3.36 | 101;126 | 3.16 | 109;330 | 2.68 | 136;279 | 7.53 | 58;330 | 1.83 |
| 203;256 | 3.12 | 187;340 | 3.36 | 101;132 | 3.16 | 22;110 | 2.68 | 54;350 | 7.53 | 185;282 | 1.83 |
| 298;338 | 3.12 | 96;217 | 3.36 | 101;131 | 3.16 | 23;138 | 2.68 | 196;277 | 7.53 | 257;338 | 1.83 |
| 298;334 | 3.12 | 96;214 | 3.36 | 101;129 | 3.16 | 343;363 | 2.68 | 184;277 | 7.53 | 257;337 | 1.83 |
| 198;298 | 3.12 | 96;215 | 3.36 | 101;128 | 3.16 | 119;356 | 2.68 | 225;276 | 7.53 | 137;280 | 1.83 |
| 252;348 | 3.12 | 96;159 | 3.36 | 62;101 | 3.16 | 34;68 | 2.68 | 124;276 | 7.53 | 137;282 | 1.83 |
| 40;316 | 3.12 | 96;216 | 3.36 | 94;101 | 3.16 | 119;357 | 2.68 | 231;276 | 7.53 | 257;331 | 1.83 |
| 193;299 | 3.12 | 96;298 | 3.36 | 101;171 | 3.16 | 23;134 | 2.68 | 113;276 | 7.53 | 58;299 | 1.83 |
| 193;298 | 3.12 | 96;220 | 3.36 | 101;181 | 3.16 | 119;359 | 2.68 | 85;277 | 7.53 | 286;350 | 1.83 |
| 40;302 | 3.12 | 23;96 | 3.36 | 101;172 | 3.16 | 149;217 | 2.68 | 145;277 | 7.53 | 58;300 | 1.83 |
| 40;300 | 3.12 | 134;187 | 3.36 | 16;101 | 3.16 | 67;119 | 2.68 | 6:277 | 7.53 | 257;318 | 1.83 |
| 40;298 | 3.12 | 134;188 | 3.36 | 101;298 | 3.16 | 94;324 | 2.68 | 103;276 | 7.53 | 194;342 | 1.83 |
| 256;317 | 3.12 | 82;96 | 3.36 | 2;101 | 3.16 | 94;321 | 2.68 | 122;276 | 7.53 | 152;354 | 1.83 |
| 294;298 | 3.12 | 132;322 | 3.36 | 101;246 | 3.16 | 23;298 | 2.68 | 91;278 | 7.53 | 35;353 | 1.83 |
| 256;337 | 3.12 | 96;204 | 3.36 | 10;101 | 3.16 | 34;362 | 2.68 | 103;278 | 7.53 | 188;257 | 1.83 |
| 293;341 | 3.12 | 68;96 | 3.36 | 104;306 | 3.16 | 34;188 | 2.68 | 188;277 | 7.53 | 94;285 | 1.83 |
| 260;358 | 3.12 | 96;181 | 3.36 | 101;309 | 3.16 | 34;363 | 2.68 | 146;277 | 7.53 | 289;337 | 1.83 |
| 293;317 | 3.12 | 96;182 | 3.36 | 104;307 | 3.16 | 34;185 | 2.68 | 86;278 | 7.53 | 94;296 | 1.83 |
| 262;308 | 3.12 | 96;183 | 3.36 | 101;315 | 3.16 | 34;181 | 2.68 | 86;277 | 7.53 | 86;337 | 1.83 |
| 191;298 | 3.12 | 229;326 | 3.36 | 101;218 | 3.16 | 34;180 | 2.68 | 147;277 | 7.53 | 86;330 | 1.83 |
| 263;302 | 3.12 | 97;147 | 3.36 | 101;291 | 3.16 | 158;298 | 2.68 | 276;312 | 7.53 | 223;282 | 1.83 |
| 292;360 | 3.12 | 96;184 | 3.36 | 101;216 | 3.16 | 119;252 | 2.68 | 61;277 | 7.53 | 134;353 | 1.83 |
| 62;308 | 3.12 | 96;185 | 3.36 | 2;173 | 3.16 | 34;217 | 2.68 | 201;279 | 7.53 | 289;299 | 1.83 |
| 193;316 | 3.12 | 97;133 | 3.36 | 2;196 | 3.16 | 34;346 | 2.68 | 239;276 | 7.53 | 154;257 | 1.83 |
| 256;298 | 3.12 | 60;96 | 3.36 | 61;101 | 3.16 | 23;68 | 2.68 | 223;279 | 7.53 | 168;342 | 1.83 |
| 195;316 | 3.12 | 85;96 | 3.36 | 2;208 | 3.16 | 74;351 | 2.68 | 202;277 | 7.53 | 154;234 | 1.83 |
| 198;300 | 3.12 | 99;326 | 3.36 | 145;208 | 3.16 | 43;126 | 2.68 | 270;278 | 7.53 | 134;285 | 1.83 |
| 207;308 | 3.12 | 96;103 | 3.36 | 101;262 | 3.16 | 119;258 | 2.68 | 222;279 | 7.53 | 86;354 | 1.83 |
| 198;269 | 3.12 | 97;137 | 3.36 | 101;217 | 3.16 | 144;298 | 2.68 | 88;357 | 7.53 | 89;280 | 1.83 |
| 197;356 | 3.12 | 96;172 | 3.36 | 101;215 | 3.16 | 136;157 | 2.68 | 222;277 | 7.53 | 86;353 | 1.83 |
| 254;304 | 3.12 | 97;203 | 3.36 | 101;173 | 3.16 | 119;249 | 2.68 | 278;313 | 7.53 | 223;353 | 1.83 |
| 254;305 | 3.12 | 98;190 | 3.36 | 101;182 | 3.16 | 135;346 | 2.68 | 278;312 | 7.53 | 223;350 | 1.83 |
| 254;308 | 3.12 | 14;326 | 3.36 | 104;145 | 3.16 | 325;351 | 2.68 | 6;276 | 7.53 | 258;338 | 1.83 |
| 196;316 | 3.12 | 96;173 | 3.36 | 101;325 | 3.16 | 119;150 | 2.68 | 83;276 | 7.53 | 289;349 | 1.83 |
| 254;338 | 3.12 | 91;326 | 3.36 | 101;186 | 3.16 | 34;171 | 2.68 | 256;276 | 7.53 | 258;337 | 1.83 |
| 254;347 | 3.12 | 96;175 | 3.36 | 101;185 | 3.16 | 34;165 | 2.68 | 49;277 | 7.53 | 258;336 | 1.83 |
| 195;308 | 3.12 | 134;356 | 3.36 | 78;306 | 3.16 | 154;252 | 2.68 | 278;309 | 7.53 | 154;233 | 1.83 |
| 193;358 | 3.12 | 96;178 | 3.36 | 101;184 | 3.16 | 34;175 | 2.68 | 168;278 | 7.53 | 35;360 | 1.83 |
| 4;308 | 3.12 | 96;179 | 3.36 | 100;104 | 3.16 | 34;157 | 2.68 | 134;279 | 7.53 | 153;299 | 1.83 |
| 294;360 | 3.12 | 96;180 | 3.36 | 99;101 | 3.16 | 59;119 | 2.68 | 104;277 | 7.53 | 35;347 | 1.83 |
| 194;342 | 3.12 | 96;294 | 3.36 | 100;101 | 3.16 | 67;343 | 2.68 | 100;276 | 7.53 | 35;350 | 1.83 |
| 294;358 | 3.12 | 96;309 | 3.36 | 101;180 | 3.16 | 23;188 | 1.74 | 225;278 | 7.53 | 153;257 | 1.83 |
| 4;316 | 3.12 | 96;307 | 3.36 | 101;178 | 3.16 | 119;154 | 1.74 | 105;276 | 7.53 | 35;358 | 1.83 |
| 256;264 | 3.12 | 96;188 | 3.36 | 101;177 | 3.16 | 23;193 | 1.74 | 102;278 | 7.53 | 236;257 | 1.83 |
| 194;316 | 3.12 | 315;326 | 3.36 | 101;175 | 3.16 | 23;252 | 1.74 | 88;277 | 7.53 | 188;252 | 1.83 |
| 294;348 | 3.12 | 96;320 | 3.36 | 101;188 | 3.16 | 237;343 | 1.74 | 37;312 | 7.53 | 188;253 | 1.83 |
| 317;335 | 3.12 | 96;321 | 3.36 | 101;193 | 3.16 | 143;211 | 1.74 | 90;276 | 7.53 | 222;338 | 1.83 |

| | | | | | | | | PAEP_MECOM positive | | PDE11A_FAM19A2 | |
|---|---|---|---|---|---|---|---|---|---|---|---|
| Metanephric cells | | Myeloid cells | | Neuroendocrine cells | | Oligodendrocytes | | cells | | positive cells | |
| Clusters | Score | Clusters | Score | Clusters | Score | Clusters | Score | Clusters | Score | Clusters | Score |
| 54;354 | 14.10 | 156;173 | 3.30 | 65;351 | 4.23 | 30;225 | 5.19 | 93;183 | 6.00 | 56;293 | 3.31 |
| 37;70 | 12.23 | 112;179 | 3.30 | 166;351 | 4.23 | 30;253 | 5.19 | 88;364 | 4.86 | 110;327 | 3.31 |
| 240;275 | 12.23 | 16;156 | 3.30 | 222;254 | 4.23 | 30;201 | 4.08 | 316;334 | 4.86 | 190;307 | 3.31 |
| 179;275 | 12.23 | 138;179 | 3.30 | 167;222 | 4.23 | 195;233 | 4.08 | 88;334 | 4.86 | 131;311 | 3.31 |
| 37;71 | 12.23 | 98;156 | 3.30 | 65;171 | 3.19 | 41;202 | 4.08 | 93;269 | 3.81 | 112;310 | 3.31 |
| 178;275 | 12.23 | 156;183 | 3.30 | 65;170 | 3.19 | 214;227 | 4.08 | 80;150 | 3.81 | 81;140 | 3.31 |
| 257;275 | 12.23 | 98;143 | 3.30 | 65;359 | 3.19 | 151;227 | 4.08 | 80;149 | 3.81 | 112;292 | 3.31 |
| 149;275 | 12.23 | 20;156 | 3.30 | 149;254 | 3.19 | 15;227 | 4.08 | 99;186 | 3.81 | 112;324 | 3.31 |
| 64;275 | 12.23 | 73;156 | 3.30 | 207;254 | 3.19 | 42;253 | 4.08 | 88;93 | 3.81 | 119;307 | 3.31 |
| 37;73 | 12.23 | 99;110 | 3.30 | 228;254 | 3.19 | 128;251 | 4.08 | 93;96 | 3.81 | 91;307 | 3.31 |
| 37;169 | 12.23 | 153;211 | 3.30 | 185;209 | 3.19 | 227;316 | 4.08 | 88;269 | 3.81 | 119;327 | 3.31 |
| 23;275 | 12.23 | 45;184 | 3.30 | 210;222 | 3.19 | 167;227 | 4.08 | 134;364 | 3.81 | 312;326 | 3.31 |
| 37;275 | 12.23 | 81;119 | 3.30 | 65;157 | 3.19 | 30;295 | 4.08 | 88;257 | 3.81 | 58;293 | 3.31 |
| 275;352 | 12.23 | 71;156 | 3.30 | 171;291 | 3.19 | 202;227 | 4.08 | 134;334 | 3.81 | 98;308 | 3.31 |
| 12;275 | 12.23 | 24;112 | 3.30 | 209;222 | 3.19 | 149;227 | 4.08 | 150;269 | 3.81 | 229;351 | 3.31 |
| 196;275 | 12.23 | 102;156 | 3.30 | 254;338 | 3.19 | 30;292 | 4.08 | 80;269 | 3.81 | 142;312 | 3.31 |
| 37;164 | 12.23 | 167;184 | 3.30 | 185;222 | 3.19 | 30;176 | 3.07 | 62;183 | 3.81 | 208;293 | 2.34 |
| 252;275 | 12.23 | 138;204 | 3.30 | 177;359 | 3.19 | 30;149 | 3.07 | 129;334 | 3.81 | 81;326 | 2.34 |
| 184;275 | 12.23 | 61;156 | 3.30 | 65;162 | 3.19 | 30;255 | 3.07 | 98;185 | 3.81 | 62;327 | 2.34 |
| 216;275 | 12.23 | 156;361 | 3.30 | 212;351 | 3.19 | 30;184 | 3.07 | 88;316 | 3.81 | 188;307 | 2.34 |
| 41;275 | 12.23 | 99;143 | 3.30 | 65;167 | 3.19 | 30;185 | 3.07 | 88;150 | 3.81 | 249;327 | 2.34 |
| 188;275 | 12.23 | 85;180 | 3.30 | 65;169 | 3.19 | 30;267 | 3.07 | 88:99 | 3.81 | 48;308 | 2.34 |
| 220;275 | 12.23 | 67;156 | 3.30 | 254;362 | 3.19 | 227;255 | 3.07 | 81;284 | 3.81 | 234;293 | 2.34 |
| 253;275 | 12.23 | 110;156 | 2.16 | 65;353 | 3.19 | 30;173 | 3.07 | 134;216 | 2.85 | 208;311 | 2.34 |
| 217;275 | 12.23 | 85;156 | 2.16 | 11;254 | 3.19 | 30;152 | 3.07 | 67;134 | 2.85 | 92;308 | 2.34 |
| 44;275 | 12.23 | 4;156 | 2.16 | 103;254 | 3.19 | 149;218 | 3.07 | 134;172 | 2.85 | 148;327 | 2.34 |
| 137;275 | 12.23 | 110;166 | 2.16 | 65;321 | 3.19 | 30;157 | 3.07 | 134;166 | 2.85 | 36;139 | 2.34 |
| 147;275 | 12.23 | 13;129 | 2.16 | 166;254 | 3.19 | 30;160 | 3.07 | 134;154 | 2.85 | 62;226 | 2.34 |
| 275;282 | 12.23 | 120;138 | 2.16 | 223;351 | 3.19 | 30;168 | 3.07 | 134;282 | 2.85 | 234;256 | 2.34 |
| 207;275 | 12.23 | 119;323 | 2.16 | 211;353 | 3.19 | 30;169 | 3.07 | 134;186 | 2.85 | 249;297 | 2.34 |
| 91;275 | 12.23 | 110;180 | 2.16 | 202;254 | 3.19 | 30;171 | 3.07 | 73;99 | 2.85 | 62;242 | 2.34 |
| 126;275 | 12.23 | 146;156 | 2.16 | 205;222 | 3.19 | 30;170 | 3.07 | 134;214 | 2.85 | 62;307 | 2.34 |
| 275;353 | 12.23 | 110;163 | 2.16 | 254;316 | 3.19 | 30;236 | 3.07 | 143;282 | 2.85 | 249;326 | 2.34 |
| 275;354 | 12.23 | 120;128 | 2.16 | 166;171 | 3.19 | 30;210 | 3.07 | 143;272 | 2.85 | 180;321 | 2.34 |
| 203;275 | 12.23 | 85;112 | 2.16 | 222;351 | 3.19 | 30;234 | 3.07 | 134;262 | 2.85 | 120;327 | 2.34 |
| 105;275 | 12.23 | 85;110 | 2.16 | 65;331 | 3.19 | 42;359 | 3.07 | 134;269 | 2.85 | 206;327 | 2.34 |
| 43;275 | 12.23 | 151;184 | 2.16 | 152;254 | 3.19 | 106;195 | 3.07 | 134;204 | 2.85 | 206;220 | 2.34 |
| 229;275 | 12.23 | 118;156 | 2.16 | 103;355 | 3.19 | 30;229 | 3.07 | 284;334 | 2.85 | 31;293 | 2.34 |
| 195;275 | 12.23 | 85;128 | 2.16 | 166;359 | 3.19 | 30;214 | 3.07 | 67;88 | 2.85 | 49;292 | 2.34 |
| 166;275 | 12.23 | 146;176 | 2.16 | 66;292 | 3.19 | 30;216 | 3.07 | 61;334 | 2.85 | 236;293 | 2.34 |
| 14;275 | 12.23 | 13;156 | 2.16 | 154;254 | 3.19 | 157;247 | 3.07 | 99;347 | 2.85 | 236;292 | 2.34 |
| 131;275 | 12.23 | 119;351 | 2.16 | 62;351 | 3.19 | 240;247 | 3.07 | 81;262 | 2.85 | 16;242 | 2.34 |
| 234;275 | 12.23 | 110;172 | 2.16 | 150;254 | 3.19 | 190;302 | 3.07 | 99;339 | 2.85 | 236;242 | 2.34 |
| 71;275 | 12.23 | 138;310 | 2.16 | 355;359 | 3.19 | 30;193 | 3.07 | 88;362 | 2.85 | 206;297 | 2.34 |
| 94;275 | 12.23 | 85;135 | 2.16 | 295;355 | 3.19 | 30;195 | 3.07 | 150;183 | 2.85 | 248;327 | 2.34 |
| 215;275 | 12.23 | 85;138 | 2.16 | 65;185 | 3.19 | 30;244 | 3.07 | 81;334 | 2.85 | 182;307 | 2.34 |
| 180;275 | 12.23 | 119;347 | 2.16 | 66;319 | 3.19 | 227;244 | 3.07 | 88;325 | 2.85 | 207;307 | 2.34 |
| 172;275 | 12.23 | 13;143 | 2.16 | 102;254 | 3.19 | 30;202 | 3.07 | 81;236 | 2.85 | 182;297 | 2.34 |
| 208;275 | 12.23 | 143;248 | 2.16 | 214;222 | 3.19 | 171;227 | 3.07 | 97;186 | 2.85 | 249;263 | 2.34 |
| 57;275 | 12.23 | 146;184 | 2.16 | 65;209 | 3.19 | 30;203 | 3.07 | 99;149 | 2.85 | 139;244 | 2.34 |
| 40;275 | 12.23 | 110;169 | 2.16 | 171;222 | 3.19 | 129;227 | 3.07 | 12;134 | 2.85 | 120;312 | 2.34 |
| 169;275 | 12.23 | 183;186 | 2.16 | 65;210 | 3.19 | 16;227 | 3.07 | 86;88 | 2.85 | 108;327 | 2.34 |
| 275;356 | 12.23 | 99;120 | 2.16 | 151;254 | 3.19 | 195;302 | 3.07 | 268;334 | 2.85 | 61;307 | 2.34 |
| 275;358 | 12.23 | 143;270 | 2.16 | 169;254 | 3.19 | 247;293 | 3.07 | 12;99 | 2.85 | 81;325 | 2.34 |
| 275;359 | 12.23 | 119;308 | 2.16 | 70;254 | 3.19 | 247;302 | 3.07 | 204;257 | 2.85 | 36;81 | 2.34 |
| 275;360 | 12.23 | 184;185 | 2.16 | 205;254 | 3.19 | 41;171 | 3.07 | 268;364 | 2.85 | 121;326 | 2.34 |
| 46;275 | 12.23 | 99;136 | 2.16 | 65;253 | 3.19 | 128;256 | 3.07 | 215;334 | 2.85 | 63;94 | 2.34 |
| 73;275 | 12.23 | 110;129 | 2.16 | 66;74 | 2.25 | 244;249 | 3.07 | 22;244 | 2.85 | 81;139 | 2.34 |
| 275;364 | 12.23 | 45;129 | 2.16 | 64;351 | 2.25 | 247;313 | 3.07 | 62;88 | 2.85 | 160;307 | 2.34 |
| 275;361 | 12.23 | 99;138 | 2.16 | 166;195 | 2.25 | 244;247 | 3.07 | 67;316 | 2.85 | 253;293 | 2.34 |
| 275;362 | 12.23 | 184;201 | 2.16 | 66:79 | 2.25 | 169;227 | 3.07 | 85:88 | 2.85 | 253;326 | 2.34 |
| 7;275 | 12.23 | 119;306 | 2.16 | 176;209 | 2.25 | 195;227 | 3.07 | 92;183 | 2.85 | 30;312 | 2.34 |
| 167;275 | 12.23 | 76;119 | 2.16 | 254;315 | 2.25 | 229;254 | 3.07 | 184;272 | 2.85 | 199;351 | 2.34 |
| 159;275 | 12.23 | 184;224 | 2.16 | 211;355 | 2.25 | 129;251 | 3.07 | 94;96 | 2.85 | 173;307 | 2.34 |
| 275;363 | 12.23 | 13;74 | 2.16 | 65;324 | 2.25 | 248;255 | 3.07 | 65;93 | 2.85 | 187;308 | 2.34 |
| 56;275 | 12.23 | 76;120 | 2.16 | 256;351 | 2.25 | 154;248 | 3.07 | 80;161 | 2.85 | 187;306 | 2.34 |
| 186;275 | 12.23 | 119;294 | 2.16 | 223;254 | 2.25 | 167;254 | 3.07 | 80;117 | 2.85 | 254;293 | 2.34 |
| 37;214 | 12.23 | 99;155 | 2.16 | 166;185 | 2.25 | 26;274 | 3.07 | 129;364 | 2.85 | 187;297 | 2.34 |
| 37;215 | 12.23 | 99;119 | 2.16 | 292;331 | 2.25 | 153;202 | 3.07 | 91;149 | 2.85 | 187;293 | 2.34 |
| 31;275 | 12.23 | 99;118 | 2.16 | 166;168 | 2.25 | 111;302 | 3.07 | 80;183 | 2.85 | 215;327 | 2.34 |
| 37;216 | 12.23 | 13;120 | 2.16 | 229;254 | 2.25 | 152;351 | 3.07 | 31;334 | 2.85 | 13;327 | 2.34 |
| 20;275 | 12.23 | 13;119 | 2.16 | 222;353 | 2.25 | 152;254 | 3.07 | 20;274 | 2.85 | 13;312 | 2.34 |
| 181;275 | 12.23 | 85;176 | 2.16 | 222;355 | 2.25 | 152;248 | 3.07 | 13;274 | 2.85 | 210;246 | 2.34 |
| 37;218 | 12.23 | 24;128 | 2.16 | 146;254 | 2.25 | 152;247 | 3.07 | 96;129 | 2.85 | 183;307 | 2.34 |
| 275;318 | 12.23 | 110;147 | 2.16 | 61;254 | 2.25 | 203;252 | 3.07 | 94;134 | 2.85 | 65;327 | 2.34 |
| 269;275 | 12.23 | 110;146 | 2.16 | 73;251 | 2.25 | 247;253 | 3.07 | 19;334 | 2.85 | 254;321 | 2.34 |
| 162;275 | 12.23 | 45;142 | 2.16 | 253;294 | 2.25 | 247;255 | 3.07 | 79;336 | 2.85 | 215;307 | 2.34 |
| 275;316 | 12.23 | 110;142 | 2.16 | 70;351 | 2.25 | 127;239 | 3.07 | 96;111 | 2.85 | 161;327 | 2.34 |
| 176;275 | 12.23 | 45;147 | 2.16 | 223;296 | 2.25 | 111;255 | 3.07 | 79;284 | 2.85 | 66;68 | 2.34 |
| 275;335 | 12.23 | 45;146 | 2.16 | 309;355 | 2.25 | 17;253 | 3.07 | 236;316 | 2.85 | 81;132 | 2.34 |
| 275;315 | 12.23 | 45;143 | 2.16 | 292;353 | 2.25 | 18;195 | 3.07 | 90;272 | 2.85 | 81;143 | 2.34 |
| 37;212 | 12.23 | 99;114 | 2.16 | 292;355 | 2.25 | 15;254 | 3.07 | 80;88 | 2.85 | 121;307 | 2.34 |
| 197;275 | 12.23 | 99;117 | 2.16 | 66;177 | 2.25 | 43;250 | 3.07 | 80;94 | 2.85 | 36;312 | 2.34 |
| 275;324 | 12.23 | 64;112 | 2.16 | 166;215 | 2.25 | 30;94 | 3.07 | 80;90 | 2.85 | 233;326 | 2.34 |
| 187;275 | 12.23 | 110;184 | 2.16 | 111;253 | 2.25 | 228;237 | 3.07 | 5;272 | 2.85 | 31;310 | 2.34 |
| 275;330 | 12.23 | 64;128 | 2.16 | 68;222 | 2.25 | 151;190 | 3.07 | 80;339 | 2.85 | 36;226 | 2.34 |
| 37;201 | 12.23 | 75;119 | 2.16 | 94;209 | 2.25 | 177;202 | 3.07 | 315;334 | 2.85 | 139;327 | 2.34 |
| 102;275 | 12.23 | 57;99 | 2.16 | 292;352 | 2.25 | 203;227 | 3.07 | 274;307 | 2.85 | 36;228 | 2.34 |
| 275;333 | 12.23 | 75;120 | 2.16 | 166;209 | 2.25 | 227;361 | 3.07 | 274;308 | 2.85 | 63;242 | 2.34 |
| 85;275 | 12.23 | 180;182 | 2.16 | 65:68 | 2.25 | 249;302 | 3.07 | 12;316 | 2.85 | 63;295 | 2.34 |
| 237;275 | 12.23 | 180;184 | 2.16 | 166;205 | 2.25 | 151;167 | 3.07 | 12;334 | 2.85 | 63;297 | 2.34 |
| 275;331 | 12.23 | 180;185 | 2.16 | 25;253 | 2.25 | 121;255 | 3.07 | 71;88 | 2.85 | 183;228 | 2.34 |
| 275;321 | 12.23 | 122;128 | 2.16 | 25;254 | 2.25 | 121;253 | 3.07 | 80;364 | 2.85 | 106;327 | 2.34 |
| 275;320 | 12.23 | 57;119 | 2.16 | 66;186 | 2.25 | 248;302 | 3.07 | 268;274 | 2.85 | 122;243 | 2.34 |
| 75;275 | 12.23 | 57;120 | 2.16 | 318;319 | 2.25 | 151;254 | 3.07 | 117;272 | 2.85 | 81;225 | 2.34 |
| 157;275 | 12.23 | 32;184 | 2.16 | 200;351 | 2.25 | 151;247 | 3.07 | 93;334 | 2.85 | 122;253 | 2.34 |
| 127;275 | 12.23 | 9;128 | 2.16 | 127;256 | 2.25 | 151;241 | 3.07 | 12;272 | 2.85 | 106;307 | 2.34 |
| 275;336 | 12.23 | 75;143 | 2.16 | 254;291 | 2.25 | 41;225 | 3.07 | 284;362 | 2.85 | 194;293 | 2.34 |
| 130;275 | 12.23 | 57;143 | 2.16 | 12;66 | 2.25 | 30;198 | 3.07 | 81;88 | 2.85 | 14;256 | 2.34 |
| 275;298 | 12.23 | 7;156 | 2.16 | 200;256 | 2.25 | 30;353 | 3.07 | 236;272 | 2.85 | 134;297 | 2.34 |

| PDE1C_ACSM3 positive | | | | | | | | Retinal progenitors and | | SATB2_LRRC7 positive | |
|---|---|---|---|---|---|---|---|---|---|---|---|
| cells | | Parietal and chief cells | | Photoreceptor cells | | Retinal pigment cells | | Muller glia | | cells | |
| Clusters | Score | Clusters | Score | Clusters | Score | Clusters | Score | Clusters | Score | Clusters | Score |
| 117;201 | 6.19 | 43;323 | 3.30 | 267;321 | 4.13 | 254;310 | 3.36 | 247;326 | 6.40 | 217;266 | 4.39 |
| 24;117 | 6.19 | 133;169 | 2.16 | 14:51 | 2.96 | 49;210 | 3.36 | 247;297 | 6.40 | 95;256 | 4.39 |
| 78;119 | 6.19 | 95;184 | 2.16 | 140;194 | 2.96 | 49;180 | 3.36 | 247;292 | 6.40 | 179;291 | 4.39 |
| 43;117 | 6.19 | 43;316 | 2.16 | 130;267 | 2.96 | 3;49 | 2.50 | 247;318 | 5.19 | 26;168 | 3.31 |
| 43;118 | 5.02 | 81;333 | 2.16 | 99;329 | 2.96 | 196;246 | 2.50 | 247;311 | 5.19 | 96;311 | 3.31 |
| 43;119 | 5.02 | 43;322 | 2.16 | 99;359 | 2.96 | 239;331 | 2.50 | 36;247 | 5.19 | 95;296 | 3.31 |
| 117;340 | 5.02 | 105;133 | 2.16 | 99;354 | 2.96 | 54;324 | 2.50 | 247;325 | 5.19 | 124;244 | 3.31 |
| 41;119 | 5.02 | 133;170 | 2.16 | 144;303 | 2.96 | 53;167 | 2.50 | 56;247 | 5.19 | 26;217 | 3.31 |
| 119;133 | 5.02 | 170;249 | 2.16 | 218;323 | 2.96 | 206;254 | 2.50 | 181;247 | 5.19 | 124;243 | 3.31 |
| 136;150 | 5.02 | 184;191 | 2.16 | 140;267 | 2.96 | 49;196 | 2.50 | 242;247 | 5.19 | 153;295 | 3.31 |
| 117;336 | 5.02 | 133;336 | 2.16 | 140;268 | 2.96 | 7;54 | 2.50 | 56;242 | 5.19 | 95;242 | 3.31 |
| 66;120 | 5.02 | 95;310 | 2.16 | 171;326 | 2.96 | 37;167 | 2.50 | 247;324 | 5.19 | 217;226 | 3.31 |
| 117;204 | 5.02 | 159;191 | 2.16 | 99;311 | 2.96 | 52;254 | 2.50 | 247;315 | 5.19 | 96;243 | 3.31 |
| 43;132 | 5.02 | 43;355 | 2.16 | 140;218 | 2.96 | 245;254 | 2.50 | 183;247 | 5.19 | 95;211 | 3.31 |
| 60;120 | 5.02 | 43;97 | 2.16 | 140;242 | 2.96 | 147;310 | 2.50 | 66;247 | 5.19 | 26;183 | 3.31 |
| 74;113 | 5.02 | 131;307 | 2.16 | 224;359 | 2.96 | 49;147 | 2.50 | 35;247 | 5.19 | 26;182 | 3.31 |
| 18;117 | 5.02 | 43;308 | 2.16 | 267;364 | 2.96 | 49;149 | 2.50 | 247;288 | 5.19 | 26;179 | 3.31 |
| 74;150 | 5.02 | 131;308 | 2.16 | 267;355 | 2.96 | 104;254 | 2.50 | 247;321 | 5.19 | 226;330 | 3.31 |
| 117;131 | 5.02 | 60;131 | 2.16 | 267;326 | 2.96 | 10;331 | 2.50 | 245;247 | 5.19 | 243;344 | 3.31 |
| 43;320 | 5.02 | 53;96 | 2.16 | 322;326 | 2.96 | 49;364 | 2.50 | 243;247 | 5.19 | 56;95 | 3.31 |
| 117;360 | 5.02 | 43;95 | 2.16 | 81;329 | 2.96 | 49;363 | 2.50 | 247;295 | 5.19 | 96;242 | 3.31 |
| 69;120 | 5.02 | 310;322 | 2.16 | 98;267 | 2.96 | 49;171 | 2.50 | 247;296 | 5.19 | 124;281 | 3.31 |
| 113;157 | 5.02 | 310;323 | 2.16 | 36;323 | 2.96 | 49;197 | 2.50 | 237;247 | 4.08 | 34;311 | 3.31 |
| 117;169 | 5.02 | 60;170 | 2.16 | 291;326 | 2.96 | 259;318 | 2.50 | 247;255 | 4.08 | 19;243 | 3.31 |
| 43;85 | 5.02 | 95;105 | 2.16 | 130;194 | 2.96 | 150;254 | 2.50 | 44;226 | 4.08 | 66;243 | 3.31 |
| 117;170 | 5.02 | 97;159 | 2.16 | 182;326 | 2.96 | 254;364 | 2.50 | 247;363 | 4.08 | 26;69 | 3.31 |
| 117;171 | 5.02 | 9;95 | 2.16 | 195;326 | 2.96 | 153;201 | 2.50 | 62;247 | 4.08 | 96;256 | 3.31 |
| 45;117 | 5.02 | 95;364 | 2.16 | 13;140 | 2.96 | 152;364 | 2.50 | 226;228 | 4.08 | 69;243 | 3.31 |
| 117;237 | 5.02 | 301;332 | 1.21 | 113;329 | 2.96 | 92;254 | 2.50 | 247;364 | 4.08 | 91;217 | 3.31 |
| 119;322 | 5.02 | 61;170 | 1.21 | 99;242 | 2.96 | 50;339 | 2.50 | 180;247 | 4.08 | 74;296 | 3.31 |
| 117;186 | 5.02 | 301;330 | 1.21 | 167;326 | 2.96 | 29;259 | 2.50 | 168;247 | 4.08 | 26;309 | 3.31 |
| 117;183 | 5.02 | 43;197 | 1.21 | 113;253 | 2.96 | 152;254 | 2.50 | 244;247 | 4.08 | 243;280 | 3.31 |
| 74;186 | 5.02 | 301;333 | 1.21 | 99;218 | 2.96 | 53;254 | 2.50 | 247;253 | 4.08 | 243;281 | 3.31 |
| 120;270 | 5.02 | 43;248 | 1.21 | 99;267 | 2.96 | 223;259 | 2.50 | 178;247 | 4.08 | 226;281 | 3.31 |
| 117;316 | 5.02 | 303;314 | 1.21 | 140;329 | 2.96 | 29;254 | 2.50 | 173;247 | 4.08 | 69;256 | 3.31 |
| 117;210 | 5.02 | 301;314 | 1.21 | 132;326 | 2.96 | 18;269 | 2.50 | 111;247 | 4.08 | 111;354 | 2.34 |
| 117;216 | 5.02 | 301;312 | 1.21 | 33;253 | 2.96 | 172;364 | 2.50 | 233;242 | 4.08 | 30;96 | 2.34 |
| 117;269 | 5.02 | 301;310 | 1.21 | 140;291 | 2.96 | 23;209 | 2.50 | 222;247 | 4.08 | 143;253 | 2.34 |
| 119;190 | 5.02 | 43;251 | 1.21 | 140;296 | 2.96 | 54;365 | 2.50 | 188;195 | 4.08 | 176;256 | 2.34 |
| 116;248 | 5.02 | 301;328 | 1.21 | 99;253 | 2.96 | 49;215 | 2.50 | 228;247 | 4.08 | 244;280 | 2.34 |
| 117;206 | 5.02 | 43;250 | 1.21 | 96;329 | 1.93 | 49;216 | 2.50 | 111;226 | 4.08 | 111;358 | 2.34 |
| 119;325 | 3.94 | 7;95 | 1.21 | 307;329 | 1.93 | 53;214 | 2.50 | 228;246 | 4.08 | 41;281 | 2.34 |
| 91;120 | 3.94 | 7:96 | 1.21 | 141;329 | 1.93 | 121;201 | 2.50 | 247;256 | 4.08 | 219;243 | 2.34 |
| 9;119 | 3.94 | 131;302 | 1.21 | 121;311 | 1.93 | 49;310 | 2.50 | 30;247 | 4.08 | 143;256 | 2.34 |
| 120;365 | 3.94 | 302;310 | 1.21 | 76;310 | 1.93 | 254;324 | 2.50 | 150;247 | 4.08 | 244;290 | 2.34 |
| 119;189 | 3.94 | 302;335 | 1.21 | 76;307 | 1.93 | 49;240 | 2.50 | 66;246 | 4.08 | 30;57 | 2.34 |
| 119;309 | 3.94 | 302;333 | 1.21 | 122;218 | 1.93 | 231;331 | 2.50 | 172;247 | 4.08 | 30;59 | 2.34 |
| 118;136 | 3.94 | 302;312 | 1.21 | 62;355 | 1.93 | 167;246 | 2.50 | 71;247 | 4.08 | 4;65 | 2.34 |
| 94;120 | 3.94 | 302;330 | 1.21 | 76;311 | 1.93 | 103;254 | 2.50 | 214;233 | 4.08 | 281;344 | 2.34 |
| 94;119 | 3.94 | 302;314 | 1.21 | 97;310 | 1.93 | 42;254 | 2.50 | 36;228 | 4.08 | 30:66 | 2.34 |
| 119;336 | 3.94 | 43;225 | 1.21 | 120;330 | 1.93 | 49;106 | 2.50 | 247;287 | 4.08 | 188;216 | 2.34 |
| 118;146 | 3.94 | 43;224 | 1.21 | 120;329 | 1.93 | 195;254 | 2.50 | 66;233 | 4.08 | 66;355 | 2.34 |
| 119;321 | 3.94 | 302;316 | 1.21 | 120;311 | 1.93 | 201;365 | 2.50 | 30;313 | 4.08 | 177;244 | 2.34 |
| 119;195 | 3.94 | 302;342 | 1.21 | 75;313 | 1.93 | 167;259 | 2.50 | 30;355 | 4.08 | 26:43 | 2.34 |
| 119;320 | 3.94 | 302;364 | 1.21 | 227;303 | 1.93 | 12;254 | 2.50 | 247;294 | 4.08 | 121;281 | 2.34 |
| 9;117 | 3.94 | 43;211 | 1.21 | 216;267 | 1.93 | 103;310 | 2.50 | 213;355 | 4.08 | 247;309 | 2.34 |
| 9;120 | 3.94 | 302;356 | 1.21 | 224;355 | 1.93 | 10;254 | 2.50 | 226;242 | 4.08 | 26:55 | 2.34 |
| 118;144 | 3.94 | 43;212 | 1.21 | 185;303 | 1.93 | 54;154 | 2.50 | 195;213 | 4.08 | 247;311 | 2.34 |
| 53;120 | 3.94 | 43;222 | 1.21 | 185;321 | 1.93 | 9;147 | 2.50 | 103;247 | 4.08 | 26;59 | 2.34 |
| 119;194 | 3.94 | 148;170 | 1.21 | 185;324 | 1.93 | 203;254 | 2.50 | 144;247 | 4.08 | 26;67 | 2.34 |
| 118;129 | 3.94 | 87;170 | 1.21 | 185;326 | 1.93 | 5;331 | 2.50 | 188;241 | 4.08 | 244;355 | 2.34 |
| 118;172 | 3.94 | 298;330 | 1.21 | 195;267 | 1.93 | 9;254 | 1.73 | 217;247 | 4.08 | 213;217 | 2.34 |
| 119;184 | 3.94 | 131;231 | 1.21 | 185;241 | 1.93 | 303;331 | 1.73 | 203;247 | 4.08 | 177;243 | 2.34 |
| 119;361 | 3.94 | 43;147 | 1.21 | 308;329 | 1.93 | 53;201 | 1.73 | 30;96 | 4.08 | 121;296 | 2.34 |
| 119;362 | 3.94 | 81;184 | 1.21 | 97;329 | 1.93 | 62;65 | 1.73 | 172;246 | 4.08 | 143;311 | 2.34 |
| 119;363 | 3.94 | 298;310 | 1.21 | 62;253 | 1.93 | 53;209 | 1.73 | 65;247 | 4.08 | 212;265 | 2.34 |
| 119;364 | 3.94 | 7:326 | 1.21 | 218;340 | 1.93 | 38;329 | 1.73 | 212;247 | 4.08 | 29;256 | 2.34 |
| 15;120 | 3.94 | 60;208 | 1.21 | 97;267 | 1.93 | 151;254 | 1.73 | 247;350 | 4.08 | 108;243 | 2.34 |
| 120;121 | 3.94 | 60;206 | 1.21 | 194;224 | 1.93 | 150;355 | 1.73 | 176;247 | 4.08 | 15;296 | 2.34 |
| 120;122 | 3.94 | 60;201 | 1.21 | 125;303 | 1.93 | 147;229 | 1.73 | 131;247 | 4.08 | 55;96 | 2.34 |
| 86;118 | 3.94 | 60;196 | 1.21 | 218;326 | 1.93 | 53;182 | 1.73 | 220;247 | 4.08 | 189;344 | 2.34 |
| 120;125 | 3.94 | 131;212 | 1.21 | 125;324 | 1.93 | 113;196 | 1.73 | 42;247 | 4.08 | 114;344 | 2.34 |
| 120;126 | 3.94 | 60;224 | 1.21 | 142;218 | 1.93 | 90;269 | 1.73 | 30;310 | 4.08 | 243;312 | 2.34 |
| 120;127 | 3.94 | 148;314 | 1.21 | 97;261 | 1.93 | 50;254 | 1.73 | 239;247 | 4.08 | 59;281 | 2.34 |
| 118;135 | 3.94 | 206;302 | 1.21 | 125;327 | 1.93 | 178;201 | 1.73 | 151;247 | 4.08 | 114;281 | 2.34 |
| 43;157 | 3.94 | 36;326 | 1.21 | 303;307 | 1.93 | 178;180 | 1.73 | 173;246 | 4.08 | 182;291 | 2.34 |
| 119;339 | 3.94 | 131;258 | 1.21 | 303;310 | 1.93 | 11;331 | 1.73 | 187;247 | 4.08 | 171;217 | 2.34 |
| 94;118 | 3.94 | 43;170 | 1.21 | 97;218 | 1.93 | 46;254 | 1.73 | 218;247 | 4.08 | 182;292 | 2.34 |
| 67;120 | 3.94 | 43;167 | 1.21 | 125;140 | 1.93 | 150;331 | 1.73 | 247;310 | 4.08 | 182;294 | 2.34 |
| 67;119 | 3.94 | 131;251 | 1.21 | 143;311 | 1.93 | 113;201 | 1.73 | 247;309 | 4.08 | 124;242 | 2.34 |
| 69;116 | 3.94 | 131;250 | 1.21 | 218;362 | 1.93 | 150;321 | 1.73 | 247;269 | 4.08 | 153;243 | 2.34 |
| 119;186 | 3.94 | 300;312 | 1.21 | 218;360 | 1.93 | 210;254 | 1.73 | 247;308 | 4.08 | 59;312 | 2.34 |
| 118;168 | 3.94 | 300;333 | 1.21 | 218;241 | 1.93 | 29;290 | 1.73 | 154;247 | 4.08 | 153;255 | 2.34 |
| 15;119 | 3.94 | 300;310 | 1.21 | 142;355 | 1.93 | 145;364 | 1.73 | 247;355 | 4.08 | 243;331 | 2.34 |
| 119;185 | 3.94 | 43;175 | 1.21 | 142;354 | 1.93 | 46;201 | 1.73 | 247;351 | 4.08 | 59;332 | 2.34 |
| 69;119 | 3.94 | 43;191 | 1.21 | 218;310 | 1.93 | 29;306 | 1.73 | 58;247 | 4.08 | 206;217 | 2.34 |
| 118;132 | 3.94 | 43;160 | 1.21 | 218;308 | 1.93 | 29;307 | 1.73 | 143;256 | 4.08 | 74;256 | 2.34 |
| 118;131 | 3.94 | 60;335 | 1.21 | 142;310 | 1.93 | 177;254 | 1.73 | 246;247 | 4.08 | 243;308 | 2.34 |
| 85;120 | 3.94 | 60;333 | 1.21 | 142;311 | 1.93 | 195;197 | 1.73 | 231;247 | 4.08 | 59;256 | 2.34 |
| 91;119 | 3.94 | 60;330 | 1.21 | 142;322 | 1.93 | 29;257 | 1.73 | 210;247 | 4.08 | 89;95 | 2.34 |
| 68;119 | 3.94 | 43;156 | 1.21 | 218;314 | 1.93 | 195;201 | 1.73 | 112;247 | 4.08 | 182;266 | 2.34 |
| 106;120 | 3.94 | 60;316 | 1.21 | 303;329 | 1.93 | 241;318 | 1.73 | 211;247 | 4.08 | 43;255 | 2.34 |
| 106;119 | 3.94 | 43;150 | 1.21 | 142;356 | 1.93 | 66;230 | 1.73 | 233;310 | 4.08 | 243;290 | 2.34 |
| 157;269 | 3.94 | 153;170 | 1.21 | 143;330 | 1.93 | 269;365 | 1.73 | 189;247 | 4.08 | 43;244 | 2.34 |
| 119;241 | 3.94 | 43;162 | 1.21 | 307;317 | 1.93 | 177;201 | 1.73 | 70;249 | 4.08 | 43;243 | 2.34 |
| 120;355 | 3.94 | 60;358 | 1.21 | 194;326 | 1.93 | 62;172 | 1.73 | 247;352 | 4.08 | 243;293 | 2.34 |
| 118;158 | 3.94 | 131;249 | 1.21 | 144;355 | 1.93 | 62;103 | 1.73 | 233;247 | 4.08 | 59;226 | 2.34 |
| 118;157 | 3.94 | 60;356 | 1.21 | 144;333 | 1.93 | 29;356 | 1.73 | 63;247 | 4.08 | 135;311 | 2.34 |
| 120;348 | 3.94 | 131;248 | 1.21 | 144;331 | 1.93 | 62;87 | 1.73 | 98;247 | 4.08 | 243;295 | 2.34 |
| 117;119 | 3.94 | 303;328 | 1.21 | 144;330 | 1.93 | 46;223 | 1.73 | 207;247 | 4.08 | 220;344 | 2.34 |

| SKOR2_NPSR1 positive | | STC2_TLX1 positive | | | | | | | | | |
|---|---|---|---|---|---|---|---|---|---|---|---|
| cells | | cells | | Satellite cells | | Schwann cells | | Skeletal muscle cells | | Smooth muscle cells | |
| Clusters | Score | Clusters | Score | Clusters | Score | Clusters | Score | Clusters | Score | Clusters | Score |
| 104;294 | 3.12 | 350;361 | 6.00 | 208;348 | 6.62 | 207;282 | 8.66 | 132;253 | 7.01 | 300;334 | 10.50 |
| 113;310 | 3.12 | 215;358 | 6.00 | 346;348 | 6.62 | 171;348 | 8.66 | 188;253 | 7.01 | 301;349 | 9.30 |
| 113;244 | 3.12 | 358;360 | 6.00 | 212;347 | 5.49 | 282;348 | 8.66 | 131;253 | 7.01 | 300;345 | 9.30 |
| 201;294 | 2.04 | 295;345 | 4.86 | 346;360 | 5.49 | 210;304 | 7.43 | 25;253 | 7.01 | 179;300 | 9.30 |
| 167;294 | 2.04 | 350;358 | 4.86 | 169;345 | 5.49 | 298;348 | 7.43 | 230;254 | 7.01 | 301;345 | 9.30 |
| 104;113 | 2.04 | 295;358 | 4.86 | 185;347 | 5.49 | 192;330 | 7.43 | 184;253 | 5.82 | 299;339 | 9.30 |
| 37;159 | 2.04 | 298;345 | 4.86 | 151;347 | 5.49 | 348;359 | 7.43 | 184;254 | 5.82 | 282;300 | 9.30 |
| 1;312 | 2.04 | 345;353 | 4.86 | 167;345 | 5.49 | 210;358 | 7.43 | 253;309 | 5.82 | 300;340 | 9.30 |
| 10;350 | 2.04 | 150;345 | 4.86 | 346;359 | 5.49 | 9;358 | 7.43 | 206;298 | 5.82 | 300;339 | 8.16 |
| 92;311 | 2.04 | 242;345 | 4.86 | 158;330 | 5.49 | 348;364 | 7.43 | 206;253 | 5.82 | 300;347 | 8.16 |
| 159;294 | 2.04 | 294;345 | 4.86 | 43;347 | 5.49 | 194;348 | 7.43 | 230;253 | 5.82 | 300;348 | 8.16 |
| 92;294 | 2.04 | 184;345 | 4.86 | 194;348 | 5.49 | 210;356 | 7.43 | 147;253 | 5.82 | 300;337 | 8.16 |
| 174;250 | 2.04 | 170;345 | 4.86 | 220;347 | 5.49 | 11;282 | 7.43 | 253;355 | 5.82 | 56;345 | 8.16 |
| 246;350 | 2.04 | 210;358 | 4.86 | 158;347 | 5.49 | 229;348 | 7.43 | 195;253 | 5.82 | 300;361 | 8.16 |
| 225;350 | 2.04 | 302;360 | 4.86 | 165;345 | 5.49 | 360;363 | 7.43 | 253;363 | 5.82 | 281;345 | 8.16 |
| 201;258 | 2.04 | 309;358 | 4.86 | 316;347 | 5.49 | 207;356 | 7.43 | 190;254 | 5.82 | 301;337 | 8.16 |
| 131;291 | 2.04 | 336;344 | 4.86 | 207;347 | 5.49 | 208;348 | 7.43 | 213;298 | 5.82 | 186;300 | 8.16 |
| 97;244 | 2.04 | 336;345 | 4.86 | 170;348 | 5.49 | 4;356 | 7.43 | 20;253 | 5.82 | 322;345 | 8.16 |
| 131;294 | 2.04 | 358;359 | 4.86 | 182;347 | 5.49 | 9;348 | 7.43 | 188;254 | 5.82 | 207;299 | 8.16 |
| 257;311 | 2.04 | 315;358 | 4.86 | 214;360 | 5.49 | 214;348 | 7.43 | 106;254 | 5.82 | 281;344 | 8.16 |
| 131;270 | 2.04 | 360;361 | 4.86 | 175;347 | 5.49 | 171;282 | 7.43 | 208;253 | 5.82 | 346;364 | 8.16 |
| 107;244 | 2.04 | 338;344 | 4.86 | 252;337 | 5.49 | 167;282 | 7.43 | 149;253 | 5.82 | 54;345 | 8.16 |
| 159;245 | 2.04 | 252;345 | 4.86 | 253;347 | 5.49 | 216;282 | 7.43 | 135;253 | 5.82 | 185;300 | 8.16 |
| 230;312 | 2.04 | 353;358 | 4.86 | 336;346 | 5.49 | 11;348 | 7.43 | 202;254 | 5.82 | 52;345 | 8.16 |
| 95;293 | 2.04 | 167;358 | 4.86 | 254;347 | 5.49 | 346;360 | 7.43 | 234;253 | 5.82 | 299;345 | 8.16 |
| 230;311 | 2.04 | 170;344 | 3.81 | 177;347 | 5.49 | 224;336 | 6.28 | 234;254 | 5.82 | 299;347 | 8.16 |
| 106;310 | 2.04 | 151;282 | 3.81 | 42;345 | 5.49 | 224;337 | 6.28 | 18;253 | 5.82 | 299;348 | 8.16 |
| 86;291 | 2.04 | 196;345 | 3.81 | 171;348 | 5.49 | 11;345 | 6.28 | 237;253 | 5.82 | 331;345 | 8.16 |
| 95;256 | 2.04 | 170;347 | 3.81 | 242;348 | 5.49 | 282;355 | 6.28 | 165;254 | 5.82 | 299;360 | 8.16 |
| 16;294 | 2.04 | 278;348 | 3.81 | 178;347 | 5.49 | 198;356 | 6.28 | 60;253 | 5.82 | 116;345 | 8.16 |
| 275;312 | 2.04 | 278;347 | 3.81 | 339;347 | 5.49 | 295;348 | 6.28 | 171;253 | 5.82 | 300;302 | 8.16 |
| 10;250 | 2.04 | 131;345 | 3.81 | 355;360 | 5.49 | 337;358 | 6.28 | 203;254 | 5.82 | 171;300 | 8.16 |
| 131;245 | 2.04 | 331;345 | 3.81 | 62;347 | 5.49 | 14;282 | 6.28 | 157;253 | 5.82 | 344;364 | 8.16 |
| 10;294 | 2.04 | 234;345 | 3.81 | 322;347 | 5.49 | 338;348 | 6.28 | 151;254 | 5.82 | 281;349 | 8.16 |
| 246;262 | 2.04 | 331;348 | 3.81 | 214;347 | 4.43 | 173;282 | 6.28 | 151;253 | 5.82 | 281;348 | 8.16 |
| 10;311 | 2.04 | 196;358 | 3.81 | 128;349 | 4.43 | 14;330 | 6.28 | 146;254 | 5.82 | 346;361 | 8.16 |
| 240;312 | 2.04 | 330;345 | 3.81 | 171;347 | 4.43 | 171;358 | 6.28 | 46;253 | 5.82 | 277;339 | 8.16 |
| 10;312 | 2.04 | 278;358 | 3.81 | 171;346 | 4.43 | 304;336 | 6.28 | 103;298 | 5.82 | 210;300 | 8.16 |
| 99;159 | 2.04 | 337;350 | 3.81 | 171;345 | 4.43 | 188;360 | 6.28 | 25;298 | 5.82 | 280;345 | 8.16 |
| 10;262 | 2.04 | 330;358 | 3.81 | 360;362 | 4.43 | 16;356 | 6.28 | 206;254 | 4.71 | 90;345 | 8.16 |
| 92;262 | 2.04 | 278;345 | 3.81 | 242;347 | 4.43 | 350;359 | 6.28 | 19;253 | 4.71 | 321;344 | 8.16 |
| 107;293 | 2.04 | 331;358 | 3.81 | 345;346 | 4.43 | 171;304 | 6.28 | 254;316 | 4.71 | 64;300 | 8.16 |
| 160;230 | 2.04 | 214;358 | 3.81 | 338;346 | 4.43 | 304;357 | 6.28 | 205;298 | 4.71 | 227;345 | 8.16 |
| 144;294 | 2.04 | 111;345 | 3.81 | 242;345 | 4.43 | 282;340 | 6.28 | 18;254 | 4.71 | 281;350 | 7.07 |
| 21;201 | 2.04 | 19;345 | 3.81 | 360;361 | 4.43 | 228;282 | 6.28 | 254;282 | 4.71 | 344;356 | 7.07 |
| 159;312 | 2.04 | 171;345 | 3.81 | 241;347 | 4.43 | 227;337 | 6.28 | 18;298 | 4.71 | 300;309 | 7.07 |
| 269;294 | 2.04 | 270;348 | 3.81 | 72;345 | 4.43 | 171;350 | 6.28 | 254;309 | 4.71 | 344;347 | 7.07 |
| 291;310 | 2.04 | 171;347 | 3.81 | 87;347 | 4.43 | 209;359 | 6.28 | 202;253 | 4.71 | 198;300 | 7.07 |
| 42;293 | 2.04 | 171;358 | 3.81 | 68;347 | 4.43 | 348;360 | 6.28 | 88;253 | 4.71 | 41;347 | 7.07 |
| 269;312 | 2.04 | 215;345 | 3.81 | 226;345 | 4.43 | 348;357 | 6.28 | 254;302 | 4.71 | 250;349 | 7.07 |
| 185;294 | 2.04 | 299;345 | 3.81 | 338;360 | 4.43 | 348;356 | 6.28 | 298;352 | 4.71 | 31;347 | 7.07 |
| 160;293 | 2.04 | 214;348 | 3.81 | 330;346 | 4.43 | 194;358 | 6.28 | 1;253 | 4.71 | 214;300 | 7.07 |
| 208;294 | 2.04 | 214;347 | 3.81 | 338;361 | 4.43 | 204;358 | 6.28 | 1;254 | 4.71 | 149;345 | 7.07 |
| 144;199 | 2.04 | 135;345 | 3.81 | 296;346 | 4.43 | 209;348 | 6.28 | 1;255 | 4.71 | 89;300 | 7.07 |
| 199;256 | 2.04 | 298;348 | 3.81 | 30;330 | 4.43 | 12;282 | 6.28 | 205;254 | 4.71 | 29;345 | 7.07 |
| 113;159 | 2.04 | 116;345 | 3.81 | 166;347 | 4.43 | 209;356 | 6.28 | 26;165 | 4.71 | 9;345 | 7.07 |
| 209;294 | 2.04 | 214;345 | 3.81 | 347;363 | 4.43 | 282;359 | 6.28 | 26;188 | 4.71 | 320;346 | 7.07 |
| 209;262 | 2.04 | 178;345 | 3.81 | 347;361 | 4.43 | 282;363 | 6.28 | 26;190 | 4.71 | 201;300 | 7.07 |
| 105;258 | 2.04 | 270;345 | 3.81 | 347;360 | 4.43 | 331;337 | 6.28 | 242;253 | 4.71 | 117;345 | 7.07 |
| 141;312 | 2.04 | 321;344 | 3.81 | 347;355 | 4.43 | 146;282 | 6.28 | 25;254 | 4.71 | 120;345 | 7.07 |
| 32;312 | 2.04 | 208;345 | 3.81 | 347;350 | 4.43 | 220;350 | 6.28 | 237;298 | 4.71 | 117;344 | 7.07 |
| 86;294 | 2.04 | 208;358 | 3.81 | 347;349 | 4.43 | 196;348 | 6.28 | 165;255 | 4.71 | 56;281 | 7.07 |
| 104;312 | 2.04 | 321;345 | 3.81 | 347;348 | 4.43 | 167;360 | 6.28 | 237;254 | 4.71 | 283;344 | 7.07 |
| 176;230 | 2.04 | 321;358 | 3.81 | 194;346 | 4.43 | 203;304 | 6.28 | 165;253 | 4.71 | 156;344 | 7.07 |
| 279;312 | 2.04 | 76;360 | 3.81 | 346;364 | 4.43 | 196;360 | 6.28 | 2;165 | 4.71 | 180;344 | 7.07 |
| 225;311 | 2.04 | 50;345 | 3.81 | 295;345 | 4.43 | 291;336 | 6.28 | 157;254 | 4.71 | 281;346 | 7.07 |
| 262;310 | 2.04 | 167;345 | 3.81 | 222;347 | 4.43 | 167;356 | 6.28 | 210;253 | 4.71 | 55;345 | 7.07 |
| 250;312 | 2.04 | 311;345 | 3.81 | 360;364 | 4.43 | 167;350 | 6.28 | 213;253 | 4.71 | 281;347 | 7.07 |
| 250;311 | 2.04 | 205;345 | 3.81 | 346;356 | 4.43 | 178;356 | 6.28 | 253;352 | 4.71 | 281;361 | 7.07 |
| 262;316 | 2.04 | 315;344 | 3.81 | 346;355 | 4.43 | 203;282 | 6.28 | 213;254 | 4.71 | 225;345 | 7.07 |
| 104;311 | 2.04 | 315;345 | 3.81 | 193;348 | 4.43 | 253;348 | 6.28 | 167;254 | 4.71 | 344;361 | 7.07 |
| 225;312 | 2.04 | 146;345 | 3.81 | 193;347 | 4.43 | 214;282 | 6.28 | 167;253 | 4.71 | 22;345 | 7.07 |
| 124;172 | 1.13 | 309;345 | 3.81 | 337;349 | 4.43 | 166;359 | 6.28 | 214;254 | 4.71 | 134;345 | 7.07 |
| 244;326 | 1.13 | 318;345 | 3.81 | 346;349 | 4.43 | 214;360 | 6.28 | 216;253 | 4.71 | 296;345 | 7.07 |
| 113;208 | 1.13 | 13;347 | 3.81 | 346;347 | 4.43 | 215;356 | 6.28 | 1;298 | 4.71 | 345;364 | 7.07 |
| 162;293 | 1.13 | 13;345 | 3.81 | 309;347 | 4.43 | 168;282 | 6.28 | 25;26 | 4.71 | 72;344 | 7.07 |
| 113;209 | 1.13 | 210;348 | 3.81 | 244;345 | 4.43 | 340;345 | 6.28 | 254;330 | 4.71 | 300;321 | 7.07 |
| 113;230 | 1.13 | 210;361 | 3.81 | 329;344 | 4.43 | 195;282 | 6.28 | 222;253 | 4.71 | 346;348 | 7.07 |
| 91;167 | 1.13 | 96;345 | 3.81 | 345;360 | 4.43 | 4;282 | 6.28 | 156;253 | 4.71 | 17;345 | 7.07 |
| 113;172 | 1.13 | 210;347 | 3.81 | 297;345 | 4.43 | 210;347 | 6.28 | 1;196 | 4.71 | 214;344 | 7.07 |
| 185;350 | 1.13 | 210;345 | 3.81 | 339;346 | 4.43 | 278;348 | 6.28 | 129;253 | 4.71 | 278;347 | 7.07 |
| 113;240 | 1.13 | 322;360 | 3.81 | 150;347 | 4.43 | 150;345 | 6.28 | 254;363 | 4.71 | 189;347 | 7.07 |
| 124;239 | 1.13 | 264;345 | 3.81 | 70;347 | 4.43 | 330;336 | 6.28 | 194;253 | 4.71 | 320;344 | 7.07 |
| 244;270 | 1.13 | 51;345 | 3.81 | 185;360 | 4.43 | 210;348 | 6.28 | 195;254 | 4.71 | 207;281 | 7.07 |
| 11;312 | 1.13 | 149;345 | 3.81 | 157;347 | 4.43 | 330;337 | 6.28 | 1;66 | 4.71 | 139;345 | 7.07 |
| 11;311 | 1.13 | 104;345 | 3.81 | 340;347 | 4.43 | 278;336 | 6.28 | 126;253 | 4.71 | 53;345 | 7.07 |
| 67;293 | 1.13 | 345;350 | 3.81 | 213;347 | 4.43 | 210;360 | 6.28 | 106;253 | 4.71 | 315;346 | 7.07 |
| 244;279 | 1.13 | 345;354 | 3.81 | 43;345 | 4.43 | 330;348 | 6.28 | 135;254 | 4.71 | 318;345 | 7.07 |
| 67;294 | 1.13 | 345;359 | 3.81 | 184;347 | 4.43 | 173;358 | 6.28 | 164;254 | 4.71 | 136;345 | 7.07 |
| 113;201 | 1.13 | 345;360 | 3.81 | 67;347 | 4.43 | 202;282 | 6.28 | 11;254 | 4.71 | 315;345 | 7.07 |
| 124;179 | 1.13 | 91;345 | 3.81 | 347;364 | 4.43 | 16;282 | 6.28 | 11;253 | 4.71 | 72;300 | 7.07 |
| 124;296 | 1.13 | 128;345 | 3.81 | 170;346 | 4.43 | 222;336 | 6.28 | 149;254 | 4.71 | 331;348 | 7.07 |
| 124;198 | 1.13 | 282;358 | 3.81 | 213;360 | 4.43 | 168;348 | 6.28 | 131;254 | 4.71 | 105;345 | 7.07 |
| 124;196 | 1.13 | 183;358 | 3.81 | 169;346 | 4.43 | 211;337 | 6.28 | 169;253 | 4.71 | 228;345 | 7.07 |
| 124;218 | 1.13 | 346;360 | 3.81 | 170;345 | 4.43 | 166;282 | 6.28 | 190;253 | 4.71 | 145;345 | 7.07 |
| 124;194 | 1.13 | 282;348 | 3.81 | 70;330 | 4.43 | 221;337 | 6.28 | 147;254 | 4.71 | 171;344 | 7.07 |
| 67;313 | 1.13 | 282;346 | 3.81 | 169;348 | 4.43 | 240;348 | 6.28 | 190;255 | 4.71 | 282;344 | 7.07 |
| 124;201 | 1.13 | 129;282 | 3.81 | 341;347 | 4.43 | 14;347 | 6.28 | 107;253 | 4.71 | 316;345 | 7.07 |
| 186;293 | 1.13 | 185;358 | 3.81 | 43;348 | 4.43 | 13;348 | 6.28 | 174;253 | 4.71 | 74;345 | 7.07 |
| 124;208 | 1.13 | 185;348 | 3.81 | 67;345 | 4.43 | 296;348 | 6.28 | 172;254 | 4.71 | 277;348 | 7.07 |

| Squamous epithelial | | | | | | | | | | | |
|---|---|---|---|---|---|---|---|---|---|---|---|
| cells | | Stellate cells | | Stromal cells | | Sympathoblasts | | Trophoblast giant cells | | Unipolar brush cells | |
| Clusters | Score | Clusters | Score | Clusters | Score | Clusters | Score | Clusters | Score | Clusters | Score |
| 77;273 | 6.03 | 301;339 | 7.22 | 299;345 | 9.99 | 251;260 | 3.60 | 175;273 | 5.79 | 64;249 | 5.18 |
| 161;273 | 6.03 | 144;301 | 7.22 | 152;299 | 9.99 | 193;254 | 3.60 | 60;81 | 5.79 | 27;173 | 5.18 |
| 273;325 | 6.03 | 10;301 | 7.22 | 176;299 | 9.99 | 254;315 | 3.60 | 214;273 | 5.79 | 65;248 | 4.25 |
| 70;273 | 6.03 | 133;301 | 7.22 | 178;299 | 9.99 | 251;297 | 3.60 | 60;79 | 5.79 | 3;292 | 4.25 |
| 221;273 | 6.03 | 263;301 | 7.22 | 72;299 | 9.99 | 169;254 | 3.60 | 152;273 | 5.79 | 3;336 | 4.25 |
| 273;336 | 6.03 | 289;301 | 7.22 | 188;299 | 9.00 | 70;251 | 3.60 | 128;273 | 5.79 | 173;249 | 4.25 |
| 9;273 | 6.03 | 277;286 | 7.22 | 300;315 | 9.00 | 238;254 | 3.60 | 80;156 | 5.79 | 4;247 | 4.25 |
| 71;273 | 6.03 | 126;301 | 7.22 | 299;331 | 9.00 | 84;254 | 3.60 | 76;273 | 5.79 | 5;65 | 4.25 |
| 14;273 | 6.03 | 290;301 | 6.17 | 159;299 | 9.00 | 251;350 | 2.56 | 60;273 | 5.79 | 193;249 | 4.25 |
| 164;273 | 6.03 | 145;301 | 6.17 | 207;299 | 9.00 | 251;356 | 2.56 | 146;273 | 5.79 | 124;219 | 4.25 |
| 183;273 | 6.03 | 24;301 | 6.17 | 299;347 | 9.00 | 151;251 | 2.56 | 273;360 | 5.79 | 4;292 | 4.25 |
| 129;273 | 6.03 | 281;302 | 6.17 | 303;304 | 9.00 | 131;251 | 2.56 | 93;156 | 5.79 | 27;67 | 4.25 |
| 150;273 | 6.03 | 186;287 | 6.17 | 185;299 | 9.00 | 211;251 | 2.56 | 169;273 | 5.79 | 52;248 | 4.25 |
| 80;282 | 6.03 | 120;301 | 6.17 | 153;299 | 9.00 | 251;357 | 2.56 | 11;273 | 5.79 | 27;152 | 3.40 |
| 170;273 | 6.03 | 28;300 | 6.17 | 299;344 | 9.00 | 6;250 | 2.56 | 167;273 | 5.79 | 27;118 | 3.40 |
| 11;273 | 6.03 | 124;301 | 6.17 | 210;299 | 9.00 | 84;351 | 2.56 | 60;340 | 5.79 | 27;169 | 3.40 |
| 219;273 | 6.03 | 25;301 | 6.17 | 225;299 | 9.00 | 152;254 | 2.56 | 156;273 | 5.79 | 27;193 | 3.40 |
| 156;273 | 6.03 | 281;301 | 6.17 | 73;299 | 9.00 | 84;362 | 2.56 | 49;273 | 4.57 | 27;206 | 3.40 |
| 184;273 | 4.75 | 108;301 | 6.17 | 361;363 | 9.00 | 158;260 | 2.56 | 273;363 | 4.57 | 242;250 | 3.40 |
| 98;273 | 4.75 | 177;301 | 6.17 | 300;361 | 9.00 | 240;251 | 2.56 | 186;273 | 4.57 | 27;334 | 3.40 |
| 273;319 | 4.75 | 119;301 | 6.17 | 209;299 | 9.00 | 158;251 | 2.56 | 60;348 | 4.57 | 27;346 | 3.40 |
| 33;273 | 4.75 | 188;301 | 6.17 | 302;348 | 9.00 | 251;360 | 2.56 | 273;307 | 4.57 | 27;53 | 3.40 |
| 64;80 | 4.75 | 300;301 | 6.17 | 72;300 | 8.04 | 84;253 | 2.56 | 60;347 | 4.57 | 64;193 | 3.40 |
| 46;273 | 4.75 | 122;301 | 6.17 | 171;299 | 8.04 | 178;255 | 2.56 | 60;338 | 4.57 | 53;221 | 3.40 |
| 10;283 | 4.75 | 102;301 | 6.17 | 302;345 | 8.04 | 251;275 | 2.56 | 60;335 | 4.57 | 65;161 | 3.40 |
| 215;273 | 4.75 | 218;301 | 6.17 | 179;299 | 8.04 | 251;272 | 2.56 | 80;105 | 4.57 | 65;193 | 3.40 |
| 212;273 | 4.75 | 87;301 | 6.17 | 282;302 | 8.04 | 171;254 | 2.56 | 60;71 | 4.57 | 65;227 | 3.40 |
| 270;273 | 4.75 | 301;361 | 6.17 | 178;300 | 8.04 | 72;84 | 2.56 | 60;78 | 4.57 | 52;64 | 3.40 |
| 124;273 | 4.75 | 277;298 | 6.17 | 230;300 | 8.04 | 171;251 | 2.56 | 60;80 | 4.57 | 65;241 | 3.40 |
| 11;80 | 4.75 | 73;301 | 6.17 | 299;362 | 8.04 | 251;255 | 2.56 | 60;93 | 4.57 | 198;248 | 3.40 |
| 152;273 | 4.75 | 149;301 | 6.17 | 299;361 | 8.04 | 87;255 | 2.56 | 48;76 | 4.57 | 95;251 | 3.40 |
| 64;272 | 4.75 | 131;301 | 6.17 | 299;360 | 8.04 | 251;253 | 2.56 | 60;105 | 4.57 | 96;251 | 3.40 |
| 64;273 | 4.75 | 85;301 | 6.17 | 176;304 | 8.04 | 87;253 | 2.56 | 60;113 | 4.57 | 118;236 | 3.40 |
| 248;273 | 4.75 | 294;301 | 6.17 | 148;299 | 8.04 | 84;286 | 2.56 | 60;147 | 4.57 | 241;249 | 3.40 |
| 22;273 | 4.75 | 302;361 | 6.17 | 299;303 | 8.04 | 178;214 | 2.56 | 221;273 | 4.57 | 193;248 | 3.40 |
| 143;283 | 4.75 | 34;301 | 6.17 | 215;299 | 8.04 | 251;315 | 2.56 | 60;148 | 4.57 | 3;181 | 3.40 |
| 283;338 | 4.75 | 85;298 | 6.17 | 11;300 | 8.04 | 84;308 | 2.56 | 60;156 | 4.57 | 3;247 | 3.40 |
| 10;80 | 4.75 | 26;301 | 6.17 | 303;344 | 8.04 | 251;296 | 2.56 | 80;273 | 4.57 | 3;200 | 3.40 |
| 262;273 | 4.75 | 175;301 | 6.17 | 299;325 | 8.04 | 84;260 | 2.56 | 60;67 | 4.57 | 3;197 | 3.40 |
| 5;273 | 4.75 | 178;287 | 6.17 | 207;300 | 8.04 | 15;84 | 2.56 | 61;359 | 4.57 | 69;251 | 3.40 |
| 63;273 | 4.75 | 280;301 | 6.17 | 132;299 | 8.04 | 210;251 | 2.56 | 172;273 | 4.57 | 3;161 | 3.40 |
| 208;283 | 4.75 | 299;301 | 6.17 | 118;299 | 8.04 | 210;254 | 2.56 | 80;118 | 4.57 | 38;248 | 3.40 |
| 18;273 | 4.75 | 118;301 | 6.17 | 299;315 | 8.04 | 216;254 | 2.56 | 61;360 | 4.57 | 3;64 | 3.40 |
| 168;273 | 4.75 | 48;301 | 6.17 | 76;299 | 8.04 | 64;84 | 2.56 | 177;273 | 4.57 | 38;251 | 3.40 |
| 273;323 | 4.75 | 12;287 | 6.17 | 13;299 | 8.04 | 198;251 | 2.56 | 60;264 | 4.57 | 3;11 | 3.40 |
| 213;283 | 4.75 | 215;277 | 6.17 | 168;299 | 8.04 | 173;254 | 2.56 | 80;165 | 4.57 | 3;353 | 3.40 |
| 273;322 | 4.75 | 25;287 | 6.17 | 104;299 | 8.04 | 198;254 | 2.56 | 80;167 | 4.57 | 3;364 | 3.40 |
| 146;283 | 4.75 | 277;301 | 6.17 | 281;299 | 8.04 | 159;255 | 2.56 | 60;274 | 4.57 | 4;64 | 3.40 |
| 173;273 | 4.75 | 148;301 | 6.17 | 302;315 | 8.04 | 73;84 | 2.56 | 60;270 | 4.57 | 249;269 | 3.40 |
| 4;273 | 4.75 | 104;301 | 6.17 | 64;299 | 8.04 | 12;254 | 2.56 | 61;214 | 4.57 | 145;251 | 3.40 |
| 273;282 | 4.75 | 31;302 | 6.17 | 299;355 | 7.12 | 173;251 | 2.56 | 188;273 | 4.57 | 6;248 | 3.40 |
| 45;273 | 4.75 | 302;363 | 6.17 | 12;299 | 7.12 | 193;251 | 2.56 | 217;273 | 4.57 | 212;250 | 3.40 |
| 154;283 | 4.75 | 246;302 | 6.17 | 303;347 | 7.12 | 12;251 | 2.56 | 198;273 | 4.57 | 227;249 | 3.40 |
| 273;278 | 4.75 | 125;301 | 6.17 | 299;356 | 7.12 | 216;251 | 2.56 | 162;273 | 4.57 | 30;73 | 3.40 |
| 75;273 | 4.75 | 111;301 | 6.17 | 111;301 | 7.12 | 28;251 | 2.56 | 60;358 | 4.57 | 30;200 | 3.40 |
| 194;273 | 4.75 | 106;301 | 6.17 | 227;299 | 7.12 | 255;351 | 2.56 | 9;273 | 4.57 | 248;343 | 3.40 |
| 170;283 | 4.75 | 301;346 | 6.17 | 299;324 | 7.12 | 33;297 | 2.56 | 242;273 | 4.57 | 4;248 | 3.40 |
| 120;283 | 4.75 | 287;301 | 6.17 | 299;358 | 7.12 | 165;251 | 2.56 | 13;60 | 4.57 | 4;212 | 3.40 |
| 66;273 | 4.75 | 179;301 | 6.17 | 304;361 | 7.12 | 255;322 | 2.56 | 60;357 | 4.57 | 73;192 | 3.40 |
| 87;273 | 4.75 | 287;298 | 6.17 | 176;303 | 7.12 | 61;251 | 2.56 | 60;354 | 4.57 | 202;248 | 3.40 |
| 104;273 | 4.75 | 289;302 | 6.17 | 76;361 | 7.12 | 61;254 | 2.56 | 273;291 | 4.57 | 202;227 | 3.40 |
| 263;273 | 4.75 | 239;301 | 6.17 | 137;299 | 7.12 | 41;84 | 2.56 | 60;253 | 3.45 | 90;250 | 3.40 |
| 155;273 | 4.75 | 301;341 | 6.17 | 76;277 | 7.12 | 254;302 | 2.56 | 25;60 | 3.45 | 251;347 | 3.40 |
| 195;273 | 4.75 | 40;301 | 6.17 | 76;348 | 7.12 | 150;255 | 2.56 | 60;252 | 3.45 | 124;236 | 3.40 |
| 12;273 | 4.75 | 143;301 | 6.17 | 94;299 | 7.12 | 79;84 | 2.56 | 60;197 | 3.45 | 92;248 | 3.40 |
| 83;273 | 4.75 | 9;287 | 6.17 | 114;300 | 7.12 | 41;251 | 2.56 | 80;186 | 3.45 | 18;226 | 3.40 |
| 105;273 | 4.75 | 301;333 | 6.17 | 203;299 | 7.12 | 97;251 | 2.56 | 80;190 | 3.45 | 58;251 | 3.40 |
| 169;273 | 4.75 | 264;301 | 6.17 | 269;299 | 7.12 | 11;251 | 2.56 | 60;185 | 3.45 | 162;250 | 3.40 |
| 103;273 | 4.75 | 301;330 | 6.17 | 72;304 | 7.12 | 253;260 | 2.56 | 80;166 | 3.45 | 201;227 | 3.40 |
| 12;80 | 4.75 | 301;326 | 6.17 | 179;304 | 7.12 | 132;251 | 2.56 | 80;169 | 3.45 | 124;357 | 3.40 |
| 179;273 | 4.75 | 301;321 | 6.17 | 196;302 | 7.12 | 152;251 | 2.56 | 60;272 | 3.45 | 251;337 | 3.40 |
| 76;273 | 4.75 | 134;301 | 6.17 | 280;344 | 7.12 | 84;236 | 2.56 | 80;174 | 3.45 | 166;248 | 3.40 |
| 73;273 | 4.75 | 75;301 | 6.17 | 178;304 | 7.12 | 254;296 | 2.56 | 60;271 | 3.45 | 227;262 | 3.40 |
| 264;273 | 4.75 | 301;315 | 6.17 | 185;300 | 7.12 | 85;251 | 2.56 | 138;272 | 3.45 | 5;67 | 2.61 |
| 153;273 | 4.75 | 49;301 | 6.17 | 299;310 | 7.12 | 85;254 | 2.56 | 50;80 | 3.45 | 5;93 | 2.61 |
| 218;273 | 4.75 | 77;299 | 6.17 | 280;303 | 7.12 | 254;260 | 2.56 | 60;267 | 3.45 | 5;92 | 2.61 |
| 106;273 | 4.75 | 286;299 | 6.17 | 203;304 | 7.12 | 55;251 | 2.56 | 44;80 | 3.45 | 5;71 | 2.61 |
| 283;331 | 4.75 | 286;300 | 6.17 | 76;300 | 7.12 | 153;253 | 2.56 | 80;189 | 3.45 | 5:70 | 2.61 |
| 11;283 | 4.75 | 132;301 | 6.17 | 131;299 | 7.12 | 144;251 | 2.56 | 60;247 | 3.45 | 201;248 | 2.61 |
| 207;273 | 4.75 | 86;301 | 6.17 | 277;303 | 7.12 | 149;260 | 2.56 | 60;216 | 3.45 | 248;291 | 2.61 |
| 172;283 | 4.75 | 286;301 | 6.17 | 175;361 | 7.12 | 149;255 | 2.56 | 60;213 | 3.45 | 169;250 | 2.61 |
| 172;273 | 4.75 | 301;304 | 6.17 | 151;299 | 7.12 | 238;358 | 2.56 | 80;221 | 3.45 | 5;38 | 2.61 |
| 273;298 | 4.75 | 137;301 | 6.17 | 298;315 | 7.12 | 224;255 | 2.56 | 60;211 | 3.45 | 5;30 | 2.61 |
| 273;294 | 4.75 | 301;348 | 6.17 | 168;300 | 7.12 | 84;118 | 2.56 | 24;359 | 3.45 | 201;247 | 2.61 |
| 273;292 | 4.75 | 262;287 | 5.18 | 300;325 | 7.12 | 118;253 | 2.56 | 80;222 | 3.45 | 4;336 | 2.61 |
| 193;273 | 4.75 | 218;287 | 5.18 | 190;299 | 7.12 | 95;253 | 2.56 | 80;224 | 3.45 | 225;236 | 2.61 |
| 171;283 | 4.75 | 213;277 | 5.18 | 290;301 | 7.12 | 218;251 | 2.56 | 60;220 | 3.45 | 4;197 | 2.61 |
| 134;273 | 4.75 | 76;301 | 5.18 | 302;347 | 7.12 | 238;251 | 2.56 | 80;226 | 3.45 | 216;248 | 2.61 |
| 171;273 | 4.75 | 299;302 | 5.18 | 56;301 | 7.12 | 250;254 | 2.56 | 80;240 | 3.45 | 249;275 | 2.61 |
| 206;283 | 4.75 | 11;301 | 5.18 | 300;345 | 7.12 | 18;84 | 2.56 | 80;220 | 3.45 | 40;200 | 2.61 |
| 283;336 | 4.75 | 277;322 | 5.18 | 213;302 | 7.12 | 32;84 | 2.56 | 60;214 | 3.45 | 90;124 | 2.61 |
| 283;320 | 4.75 | 15;301 | 5.18 | 208;299 | 7.12 | 84;202 | 2.56 | 80;241 | 3.45 | 40;251 | 2.61 |
| 80;193 | 4.75 | 299;315 | 5.18 | 300;344 | 7.12 | 84;108 | 2.56 | 60;215 | 3.45 | 164;250 | 2.61 |
| 80;280 | 4.75 | 171;301 | 5.18 | 25;301 | 7.12 | 80;251 | 2.56 | 80;248 | 3.45 | 57;250 | 2.61 |
| 229;273 | 4.75 | 293;301 | 5.18 | 298;345 | 7.12 | 5;251 | 2.56 | 80;254 | 3.45 | 57;221 | 2.61 |
| 138;273 | 4.75 | 17;301 | 5.18 | 25;300 | 7.12 | 147;254 | 2.56 | 80;253 | 3.45 | 248;364 | 2.61 |
| 80;164 | 4.75 | 256;301 | 5.18 | 301;361 | 7.12 | 84;205 | 2.56 | 24;360 | 3.45 | 90;248 | 2.61 |
| 211;273 | 4.75 | 78;301 | 5.18 | 25;299 | 7.12 | 224;253 | 2.56 | 60;200 | 3.45 | 248;352 | 2.61 |
| 80;154 | 4.75 | 8;301 | 5.18 | 21;301 | 7.12 | 64;254 | 2.56 | 60;238 | 3.45 | 5;95 | 2.61 |
| 80;150 | 4.75 | 70;301 | 5.18 | 188;304 | 7.12 | 242;251 | 2.56 | 60;201 | 3.45 | 248;250 | 2.61 |

| | | Vascular endothelial | | | |
|---|---|---|---|---|---|
| Ureteric bud cells | | cells | | Visceral neurons | |
| Clusters | Score | Clusters | Score | Clusters | Score |
| 121;271 | 5.38 | 9;305 | 34.04 | 166;202 | 4.57 |
| 67;271 | 5.38 | 73;305 | 32.44 | 184;254 | 4.57 |
| 225;271 | 5.38 | 74;305 | 32.44 | 202;254 | 4.57 |
| 46;271 | 5.38 | 172;305 | 32.44 | 185;254 | 4.57 |
| 37;264 | 5.38 | 118;305 | 32.44 | 207;254 | 3.45 |
| 212;271 | 5.38 | 72;305 | 32.44 | 251;364 | 3.45 |
| 267;272 | 4.23 | 147;305 | 32.44 | 196;202 | 3.45 |
| 264;275 | 4.23 | 305;348 | 32.44 | 67;254 | 3.45 |
| 33;271 | 4.23 | 62;305 | 32.44 | 151;253 | 3.45 |
| 43;284 | 4.23 | 171;305 | 30.87 | 186;254 | 3.45 |
| 267;273 | 4.23 | 169;305 | 30.87 | 206;254 | 3.45 |
| 87;88 | 4.23 | 12;305 | 30.87 | 150;251 | 3.45 |
| 264;284 | 4.23 | 146;305 | 30.87 | 9;252 | 3.45 |
| 47;271 | 4.23 | 213;305 | 30.87 | 9;251 | 3.45 |
| 81;271 | 4.23 | 215;305 | 30.87 | 254;294 | 3.45 |
| 271;353 | 4.23 | 129;305 | 30.87 | 252;253 | 3.45 |
| 184;271 | 4.23 | 305;340 | 30.87 | 88;254 | 3.45 |
| 202;271 | 4.23 | 64;305 | 30.87 | 220;252 | 3.45 |
| 215;271 | 4.23 | 305;338 | 30.87 | 220;251 | 3.45 |
| 20;271 | 4.23 | 174;305 | 30.87 | 213;254 | 3.45 |
| 264;273 | 4.23 | 269;305 | 30.87 | 94;253 | 3.45 |
| 92;264 | 4.23 | 305;346 | 30.87 | 185;202 | 3.45 |
| 201;264 | 4.23 | 42;305 | 30.87 | 22;251 | 3.45 |
| 79;271 | 4.23 | 179;305 | 30.87 | 166;205 | 3.45 |
| 85;271 | 4.23 | 156;305 | 29.33 | 167;178 | 3.45 |
| 273;308 | 4.23 | 210;305 | 29.33 | 166;353 | 3.45 |
| 88;361 | 4.23 | 195;305 | 29.33 | 166;351 | 3.45 |
| 236;271 | 4.23 | 305;349 | 29.33 | 166;262 | 3.45 |
| 86;271 | 4.23 | 305;339 | 29.33 | 166;254 | 3.45 |
| 92;271 | 4.23 | 155;305 | 29.33 | 166;213 | 3.45 |
| 213;271 | 4.23 | 305;364 | 29.33 | 166;210 | 3.45 |
| 46;284 | 4.23 | 305;341 | 29.33 | 166;207 | 3.45 |
| 273;297 | 4.23 | 305;309 | 29.33 | 166;181 | 3.45 |
| 89;271 | 4.23 | 214;305 | 29.33 | 166;167 | 3.45 |
| 214;271 | 4.23 | 178;305 | 29.33 | 165;251 | 3.45 |
| 267;271 | 4.23 | 305;362 | 29.33 | 160;251 | 3.45 |
| 266;271 | 4.23 | 157;305 | 29.33 | 159;254 | 3.45 |
| 39;271 | 4.23 | 71;305 | 29.33 | 159;251 | 3.45 |
| 237;271 | 4.23 | 25;305 | 29.33 | 157;251 | 3.45 |
| 257;271 | 4.23 | 185;305 | 29.33 | 156;294 | 3.45 |
| 39;283 | 4.23 | 305;325 | 29.33 | 23;251 | 3.45 |
| 60;264 | 4.23 | 103;305 | 29.33 | 184;353 | 3.45 |
| 273;275 | 4.23 | 305;324 | 29.33 | 184;251 | 3.45 |
| 275;283 | 4.23 | 105;305 | 29.33 | 152;254 | 3.45 |
| 186;271 | 4.23 | 16;305 | 29.33 | 65;166 | 3.45 |
| 88;266 | 4.23 | 173;305 | 29.33 | 65;152 | 3.45 |
| 93;284 | 4.23 | 67;305 | 29.33 | 181;254 | 3.45 |
| 16;271 | 4.23 | 206;305 | 29.33 | 178;202 | 3.45 |
| 182;271 | 4.23 | 184;305 | 29.33 | 173;254 | 3.45 |
| 153;271 | 4.23 | 170;305 | 29.33 | 63;251 | 3.45 |
| 209;271 | 4.23 | 86;305 | 29.33 | 251;297 | 3.45 |
| 264;265 | 3.19 | 61;305 | 29.33 | 251;293 | 3.45 |
| 51;271 | 3.19 | 168;305 | 29.33 | 251;287 | 3.45 |
| 273;325 | 3.19 | 196;305 | 29.33 | 251;257 | 3.45 |
| 273;326 | 3.19 | 166;305 | 29.33 | 251;288 | 3.45 |
| 23;284 | 3.19 | 164;305 | 29.33 | 251;253 | 3.45 |
| 83;267 | 3.19 | 208;305 | 29.33 | 235;251 | 2.45 |
| 211;264 | 3.19 | 305;347 | 27.82 | 181;251 | 2.45 |
| 271;307 | 3.19 | 305;353 | 27.82 | 65;103 | 2.45 |
| 264;266 | 3.19 | 305;316 | 27.82 | 250;256 | 2.45 |
| 284;306 | 3.19 | 305;350 | 27.82 | 181;353 | 2.45 |
| 35;271 | 3.19 | 209;305 | 27.82 | 177;251 | 2.45 |
| 264;267 | 3.19 | 43;305 | 27.82 | 65;149 | 2.45 |
| 83;264 | 3.19 | 204;305 | 27.82 | 177;253 | 2.45 |
| 183;284 | 3.19 | 212;305 | 27.82 | 65;154 | 2.45 |
| 63;271 | 3.19 | 305;320 | 27.82 | 183;251 | 2.45 |
| 211;257 | 3.19 | 197;305 | 27.82 | 64;167 | 2.45 |
| 268;284 | 3.19 | 70;305 | 27.82 | 183;254 | 2.45 |
| 60;271 | 3.19 | 305;343 | 27.87 | 65;171 | 2.45 |
| 131;271 | 3.19 | 305;321 | 27.82 | 181;250 | 2.45 |
| 258;271 | 3.19 | 305;337 | 27.82 | 178;253 | 2.45 |
| 273;351 | 3.19 | 207;305 | 27.82 | 110;251 | 2.45 |
| 183;271 | 3.19 | 216;305 | 27.82 | 177;254 | 2.45 |
| 60;275 | 3.19 | 228;305 | 27.82 | 181;202 | 2.45 |
| 268;274 | 3.19 | 152;305 | 27.82 | 251;296 | 2.45 |
| 23;271 | 3.19 | 159;305 | 27.82 | 105;253 | 2.45 |
| 23;273 | 3.19 | 98;305 | 27.82 | 184;193 | 2.45 |
| 153;264 | 3.19 | 183;305 | 27.82 | 187;251 | 2.45 |
| 275;343 | 3.19 | 88;305 | 27.82 | 104;251 | 2.45 |
| 200;271 | 3.19 | 130;305 | 27.82 | 103;292 | 2.45 |
| 46;272 | 3.19 | 252;305 | 27.82 | 103;254 | 2.45 |
| 211;272 | 3.19 | 186;305 | 27.82 | 103;253 | 2.45 |
| 124;271 | 3.19 | 167;305 | 27.82 | 103;251 | 2.45 |
| 284;307 | 3.19 | 2;305 | 27.82 | 103;250 | 2.45 |
| 211;271 | 3.19 | 149;305 | 27.82 | 191;251 | 2.45 |
| 164;271 | 3.19 | 161;305 | 27.82 | 192;251 | 2.45 |
| 168;273 | 3.19 | 190;305 | 27.82 | 192;254 | 2.45 |
| 105;275 | 3.19 | 44;305 | 27.82 | 250;343 | 2.45 |
| 271;325 | 3.19 | 154;305 | 27.82 | 65;343 | 2.45 |
| 81;284 | 3.19 | 191;305 | 27.82 | 230;251 | 2.45 |
| 78;264 | 3.19 | 202;305 | 27.82 | 186;253 | 2.45 |
| 180;271 | 3.19 | 305;310 | 27.82 | 13;251 | 2.45 |
| 9;284 | 3.19 | 229;305 | 27.82 | 176;308 | 2.45 |
| 81;272 | 3.19 | 15;305 | 27.82 | 65;186 | 2.45 |
| 188;271 | 3.19 | 194;305 | 27.82 | 232;251 | 2.45 |
| 174;275 | 3.19 | 201;305 | 27.82 | 65;205 | 2.45 |
| 81;275 | 3.19 | 17;305 | 27.82 | 184;233 | 2.45 |
| 273;333 | 3.19 | 305;363 | 27.82 | 5;251 | 2.45 |
| 168;271 | 3.19 | 45;305 | 27.82 | 13;233 | 2.45 |
| 271;323 | 3.19 | 160;305 | 27.82 | 184;292 | 2.45 |

TABLE 19C
Hierarchical clustering of TF ORFs for TF Atlas differentiated cells into 151 clusters.

| | Clus- | | Clus- | | Clus- | | Clus- |
|---|---|---|---|---|---|---|---|
| TF ORF | ter | TF ORF | ter | TF ORF | ter | TF ORF | ter |
| TFORF0483-ZNF223 | 1 | TFORF0306-CREM | 54 | TFORF0048-MAX | 84 | TFORF0998-ZNF302 | 123 |
| TFORF0501-EGR4 | 1 | TFORF0394-ZNF7 | 54 | TFORF0076-KCNIP4 | 84 | TFORF1191-ZNF573 | 123 |
| TFORF0524-PAX4 | 1 | TFORF0533-PAX1 | 54 | TFORF0081-KCNIP2 | 84 | TFORF1724-ZNF691 | 123 |
| TFORF0606-PUF60 | 1 | TFORF0666-TGFB1I1 | 54 | TFORF0128-SP8 | 84 | TFORF1917-RUNX2 | 123 |
| TFORF0974-GFI1B | 1 | TFORF0874-HOXB9 | 54 | TFORF0131-HMGB4 | 84 | TFORF1973-TBX3 | 123 |
| TFORF1157-DMBX1 | 1 | TFORF1044-TSC22D3 | 54 | TFORF0238-MYCL | 84 | TFORF2078-FLI1 | 123 |
| TFORF1412-ZNF562 | 1 | TFORF1366-ISL2 | 54 | TFORF0244-SUB1 | 84 | TFORF2766-ZKSCAN7 | 123 |
| TFORF1479-TP63 | 1 | TFORF1955-ESR2 | 54 | TFORF0252-CAMTA1 | 84 | TFORF0005-HIF3A | 124 |
| TFORF1592-ZNF771 | 1 | TFORF2162-GRHL1 | 54 | TFORF0304-CREM | 84 | TFORF0269-TCF4 | 124 |
| TFORF1923-ZBTB20 | 1 | TFORF2252-ZNF630 | 54 | TFORF0365-TCEB1 | 84 | TFORF0646-NR1I3 | 124 |
| TFORF2132-LCOR | 1 | TFORF2404-NFE2L1 | 54 | TFORF0374-NFYA | 84 | TFORF0768-ZNF551 | 124 |
| TFORF2467-IRF5 | 1 | TFORF2423-RBPJL | 54 | TFORF0399-ZNF2 | 84 | TFORF0961-ERG | 124 |
| TFORF2682-ZMYND11 | 1 | TFORF2631-INSM1 | 54 | TFORF0503-RNF138 | 84 | TFORF1612-TADA2A | 124 |
| TFORF2706-TFCP2L1 | 1 | TFORF2689-TEAD2 | 54 | TFORF0600-MZF1 | 84 | TFORF1757-ZNF189 | 124 |
| TFORF3344-ZNF280A | 1 | TFORF3306-THAP1 | 54 | TFORF0647-NR1I3 | 84 | TFORF1760-ZNF25 | 124 |
| TFORF0292-MEF2B | 2 | TFORF3308-GTF2A1L | 54 | TFORF0741-JDP2 | 84 | TFORF2730-ZNF668 | 124 |
| TFORF0519-PAX5 | 2 | TFORF3364-ZNF232 | 54 | TFORF0771-MSX2 | 84 | TFORF2913-NFIB | 124 |
| TFORF0738-ZNF155 | 2 | TFORF3384-HOXC10 | 54 | TFORF0801-ATF3 | 84 | TFORF3113-RFXANK | 124 |
| TFORF0853-ZIC5 | 2 | TFORF3416-ZNF410 | 54 | TFORF0942-LMO1 | 84 | TFORF3231-ZNF423 | 124 |
| TFORF1030-RARG | 2 | TFORF3433-DEK | 54 | TFORF0992-KLF8 | 84 | TFORF3503-CDK7 | 124 |
| TFORF1165-PRKCD | 2 | TFORF3506-LCOR | 54 | TFORF1022-SCRT2 | 84 | TFORF0045-ZNF671 | 125 |
| TFORF1273-DPF2 | 2 | TFORF0362-TCEB2 | 55 | TFORF1040-TSC22D1 | 84 | TFORF0601-ZNF726 | 125 |
| TFORF1370-ZNF141 | 2 | TFORF1039-TSC22D1 | 55 | TFORF1219-PRRX2 | 84 | TFORF1869-POU2F1 | 125 |
| TFORF2107-ZBTB37 | 2 | TFORF1291-SP140L | 55 | TFORF1494-FOSL1 | 84 | TFORF2235-ZNF880 | 125 |
| TFORF2328-CIZ1 | 2 | TFORF1387-ZNF367 | 55 | TFORF1556-YAF2 | 84 | TFORF2788-OTX2 | 125 |
| TFORF2333-ZNF491 | 2 | TFORF2131-ZGLP1 | 55 | TFORF2114-SIRT6 | 84 | TFORF2801-ETV2 | 125 |
| TFORF2411-KLF3 | 2 | TFORF2354-ZNF12 | 55 | TFORF2121-HOPX | 84 | TFORF3110-TGIF2LY | 125 |
| TFORF2855-MAPK8IP1 | 2 | TFORF2444-SMARCE1 | 55 | TFORF2144-ZNF525 | 84 | TFORF3302-SPIC | 125 |
| TFORF3109-ELF5 | 2 | TFORF2829-IKZF3 | 55 | TFORF2215-CREB3L4 | 84 | TFORF0015-DRAP1 | 126 |
| TFORF3137-HEY1 | 2 | TFORF3016-MLX | 55 | TFORF2250-TCF19 | 84 | TFORF0486-ZFP90 | 126 |
| TFORF0054-ZNF679 | 3 | TFORF3466-SIM2 | 55 | TFORF2253-HMGN3 | 84 | TFORF0640-NHP2 | 126 |
| TFORF0843-ZSCAN10 | 3 | TFORF3489-IKZF1 | 55 | TFORF2294-TAF13 | 84 | TFORF0892-ING4 | 126 |
| TFORF0849-ZIC4 | 3 | TFORF0426-PBX3 | 56 | TFORF2418-ZNF268 | 84 | TFORF1009-ZNF544 | 126 |
| TFORF1362-SP100 | 3 | TFORF0516-PAX5 | 56 | TFORF2516-PDCD2 | 84 | TFORF1128-THAP3 | 126 |
| TFORF1535-ZBTB44 | 3 | TFORF1126-THAP3 | 56 | TFORF2561-ZNF655 | 84 | TFORF1158-EOMES | 126 |
| TFORF0478-ZNF222 | 4 | TFORF1221-HELT | 56 | TFORF2586-CERS4 | 84 | TFORF1363-ELOF1 | 126 |
| TFORF0514-PAX5 | 4 | TFORF2152-BCL11A | 56 | TFORF2857-ID4 | 84 | TFORF1409-ZNF559 | 126 |
| TFORF0515-PAX5 | 4 | TFORF2319-TAF1B | 56 | TFORF3000-E2F6 | 84 | TFORF1515-NANOGNB | 126 |
| TFORF0517-PAX5 | 4 | TFORF2569-TGIF1 | 56 | TFORF3142-CDX4 | 84 | TFORF1714-LDB2 | 126 |
| TFORF0518-PAX5 | 4 | TFORF2654-ARNTL2 | 56 | TFORF3207-HMGN2 | 84 | TFORF1947-MIER1 | 126 |
| TFORF0537-PAX2 | 4 | TFORF1649-REL | 57 | TFORF3274-HMGA1 | 84 | TFORF2311-RXRG | 126 |
| TFORF0538-PAX2 | 4 | TFORF1884-ZIC2 | 57 | TFORF3294-ZNF784 | 84 | TFORF2633-TFDP2 | 126 |
| TFORF0552-ZNF611 | 4 | TFORF2350-ZNF16 | 57 | TFORF3530-SSRP1 | 84 | TFORF2705-ESRRB | 126 |
| TFORF0742-ZNF485 | 4 | TFORF2576-HSF4 | 57 | TFORF3535-MLLT10 | 84 | TFORF2725-ZNF74 | 126 |
| TFORF1277-ZNF587 | 4 | TFORF2800-TAF5 | 57 | TFORF0084-KCNIP2 | 85 | TFORF2803-ETV2 | 126 |
| TFORF1302-ZMIZ2 | 4 | TFORF3141-ALX3 | 57 | TFORF0183-SHOX2 | 85 | TFORF3033-NFKBIA | 126 |
| TFORF1921-ZBTB22 | 4 | TFORF3147-ZBTB43 | 57 | TFORF0291-MEF2C | 85 | TFORF3476-SNAI1 | 126 |
| TFORF1981-TBX4 | 4 | TFORF3266-ZNF213 | 57 | TFORF0332-ZNF333 | 85 | TFORF3512-SOX14 | 126 |
| TFORF2225-ZNF343 | 4 | TFORF3482-SOX2 | 57 | TFORF0349-ZNF48 | 85 | TFORF0007-TULP4 | 127 |
| TFORF2501-PML | 4 | TFORF0826-LHX9 | 58 | TFORF0718-TEF | 85 | TFORF0135-ZNF780A | 127 |
| TFORF2882-XBP1 | 4 | TFORF1099-UBP1 | 58 | TFORF0938-SS18 | 85 | TFORF0812-CDCA7L | 127 |
| TFORF3201-ZNF829 | 4 | TFORF1516-ZNF737 | 58 | TFORF0949-EMX1 | 85 | TFORF0860-NR1H4 | 127 |
| TFORF3418-NFE2L2 | 4 | TFORF1664-ARID5A | 58 | TFORF1255-TP53 | 85 | TFORF1159-EOMES | 127 |
| TFORF3504-ZNF543 | 4 | TFORF1878-CCNT2 | 58 | TFORF1407-ZNF559 | 85 | TFORF1181-TBX21 | 127 |
| TFORF0165-SIX5 | 5 | TFORF2035-OSR2 | 58 | TFORF1454-TFEC | 85 | TFORF1372-ZNF143 | 127 |
| TFORF0336-ZNF91 | 5 | TFORF3259-MEF2D | 58 | TFORF1551-TAZ | 85 | TFORF1376-ZNF146 | 127 |
| TFORF0397-ZNF2 | 5 | TFORF3494-OSR2 | 58 | TFORF1590-ZNF773 | 85 | TFORF1473-LIN54 | 127 |
| TFORF0507-CDK2 | 5 | TFORF0279-ZNF776 | 59 | TFORF1643-ZNF426 | 85 | TFORF1603-HINFP | 127 |
| TFORF0698-BATF2 | 5 | TFORF0985-KLF4 | 59 | TFORF1846-ETS1 | 85 | TFORF1940-JUP | 127 |
| TFORF0837-ZSCAN18 | 5 | TFORF1246-ZNF133 | 59 | TFORF1915-RUNX2 | 85 | TFORF2099-SOHLH2 | 127 |
| TFORF0901-ZNF382 | 5 | TFORF1298-ZFP64 | 59 | TFORF1997-ZNF248 | 85 | TFORF2370-MBD4 | 127 |
| TFORF1325-KLF16 | 5 | TFORF1357-ILF3 | 59 | TFORF2246-TCF12 | 85 | TFORF2405-NFE2L1 | 127 |
| TFORF1421-ZNF211 | 5 | TFORF1725-ZNF691 | 59 | TFORF2254-HMGN3 | 85 | TFORF2515-PDCD2 | 127 |
| TFORF1554-SP110 | 5 | TFORF3154-ZFP2 | 59 | TFORF2288-HES4 | 85 | TFORF2741-ZNF484 | 127 |
| TFORF2181-ZNF417 | 5 | TFORF3411-CRTC2 | 59 | TFORF2974-NR1H2 | 85 | TFORF2885-GLI2 | 127 |
| TFORF2188-ZNF410 | 5 | TFORF0192-RFX4 | 60 | TFORF3321-PA2G4 | 85 | TFORF3007-SMAD1 | 127 |
| TFORF2214-CREB3L4 | 5 | TFORF0325-LCORL | 60 | TFORF0359-TUB | 86 | TFORF3379-ACTL6A | 127 |
| TFORF2584-MIXL1 | 5 | TFORF0589-FOXO1 | 60 | TFORF0428-PBX1 | 86 | TFORF3425-ZNF559 | 127 |
| TFORF2942-ESRRG | 5 | TFORF0868-NR1H3 | 60 | TFORF0648-NR1I3 | 86 | TFORF0182-SHOX2 | 128 |
| TFORF3324-XRCC6 | 5 | TFORF1123-HNRNPAB | 60 | TFORF0756-JUND | 86 | TFORF0591-FOXO4 | 128 |
| TFORF3441-MNAT1 | 5 | TFORF1609-ZZZ3 | 60 | TFORF0800-ATF3 | 86 | TFORF0999-ZNF302 | 128 |
| TFORF0324-LCORL | 6 | TFORF1860-NR4A3 | 60 | TFORF0883-HOXB7 | 86 | TFORF1054-PLAGL1 | 128 |
| TFORF0398-ZNF2 | 6 | TFORF1960-CUX1 | 60 | TFORF2480-MXD4 | 86 | TFORF1141-HMBOX1 | 128 |
| TFORF0464-TRIM22 | 6 | TFORF1962-CUX1 | 60 | TFORF2947-HOXC8 | 86 | TFORF1322-KLF10 | 128 |
| TFORF0523-PAX5 | 6 | TFORF2186-ZNF414 | 60 | TFORF0057-MAF | 87 | TFORF1613-TADA2A | 128 |
| TFORF0588-FOXO3 | 6 | TFORF2466-IRF5 | 60 | TFORF0152-THAP6 | 87 | TFORF1759-ALYREF | 128 |
| TFORF0610-RBPJ | 6 | TFORF2635-TFDP2 | 60 | TFORF0436-ZSCAN31 | 87 | TFORF1816-SATB1 | 128 |
| TFORF1000-ZNF302 | 6 | TFORF2798-TAF6 | 60 | TFORF0696-GTF3A | 87 | TFORF2069-MYC | 128 |
| TFORF1051-HKR1 | 6 | TFORF2915-NFIB | 60 | TFORF0781-ZNF93 | 87 | TFORF2150-ZNF250 | 128 |
| TFORF1081-NOBOX | 6 | TFORF2933-HOXD3 | 60 | TFORF1562-ZNF688 | 87 | TFORF2303-CIITA | 128 |
| TFORF1341-POU5F1 | 6 | TFORF3138-PPARG | 60 | TFORF1934-MEIS2 | 87 | TFORF2784-GLIS3 | 128 |
| TFORF1493-FOSL1 | 6 | TFORF3230-FOXJ1 | 60 | TFORF2446-ZIK1 | 87 | TFORF3029-ATOH1 | 128 |
| TFORF1680-GTF2IRD2B | 6 | TFORF3345-LMO3 | 60 | TFORF2757-BARHL1 | 87 | TFORF3087-MAFF | 128 |
| TFORF1806-TBX15 | 6 | TFORF3386-IRF4 | 60 | TFORF3337-HDAC1 | 87 | TFORF3267-SNAPC2 | 128 |
| TFORF1849-ETS1 | 6 | TFORF3480-ZNF785 | 60 | TFORF0270-TCF7 | 88 | TFORF3283-HOXA5 | 128 |
| TFORF1930-XRCC6 | 6 | TFORF0082-KCNIP2 | 61 | TFORF0367-NFYC | 88 | TFORF3374-VAX2 | 128 |
| TFORF1936-MEIS2 | 6 | TFORF0085-KCNIP2 | 61 | TFORF0894-ING4 | 88 | TFORF3521-THAP8 | 128 |
| TFORF1987-ZNF534 | 6 | TFORF0240-MYCL | 61 | TFORF1393-PMS1 | 88 | TFORF0032-GSC2 | 129 |
| TFORF2124-ZNF747 | 6 | TFORF0267-TCF4 | 61 | TFORF1485-NANOG | 88 | TFORF0230-ZNF595 | 129 |
| TFORF2340-MITF | 6 | TFORF0505-CDK2 | 61 | TFORF2111-NR4A1 | 88 | TFORF0654-NR1I3 | 129 |
| TFORF2372-ZNF468 | 6 | TFORF0546-ZFAT | 61 | TFORF2349-HOXD8 | 88 | TFORF0854-VSX1 | 129 |
| TFORF2513-PDCD2 | 6 | TFORF0611-RBPJ | 61 | TFORF2474-ONECUT3 | 88 | TFORF1182-TBX20 | 129 |
| TFORF2621-ZXDA | 6 | TFORF0618-ZSCAN26 | 61 | TFORF3146-HSF1 | 88 | TFORF1481-TP63 | 129 |
| TFORF2970-RORC | 6 | TFORF0766-ZNF552 | 61 | TFORF3193-DND1 | 88 | TFORF2287-HES4 | 129 |
| TFORF3012-HSF2 | 6 | TFORF0840-HSFY2 | 61 | TFORF3349-ERCC8 | 88 | TFORF2382-MBD1 | 129 |
| TFORF3222-FOXS1 | 6 | TFORF1215-NME2 | 61 | TFORF0017-IRX5 | 89 | TFORF2590-CERS3 | 129 |
| TFORF3385-ZIC3 | 6 | TFORF1382-ZNF789 | 61 | TFORF0109-SMAD6 | 89 | TFORF2859-ID1 | 129 |
| TFORF3464-KLF7 | 6 | TFORF1836-EZH2 | 61 | TFORF0206-ETV3L | 89 | TFORF2870-HMX1 | 129 |
| TFORF3500-BATF3 | 6 | TFORF1850-YWHAE | 61 | TFORF0525-PAX4 | 89 | TFORF3251-PFDN5 | 129 |
| TFORF0046-MAX | 7 | TFORF2142-ZNF525 | 61 | TFORF0570-ZNF502 | 89 | TFORF0467-CARHSP1 | 130 |
| TFORF0630-NR1I2 | 7 | TFORF2204-MYNN | 61 | TFORF0879-HOXB3 | 89 | TFORF1086-GTF2E1 | 130 |
| TFORF0632-VPS72 | 7 | TFORF2239-GATA5 | 61 | TFORF1160-EOMES | 89 | TFORF1094-HSFX2 | 130 |
| TFORF0835-HSFY1 | 7 | TFORF2414-ZNF268 | 61 | TFORF1425-ZNF211 | 89 | TFORF1330-TFAP2C | 130 |
| TFORF1256-TP53 | 7 | TFORF2585-CERS6 | 61 | TFORF2205-POU2F3 | 89 | TFORF1487-NR6A1 | 130 |
| TFORF1299-MAEL | 7 | TFORF2664-ZBED1 | 61 | TFORF2599-RORA | 89 | TFORF1640-PSIP1 | 130 |
| TFORF1880-CREBZF | 7 | TFORF2716-SLC2A4RG | 61 | TFORF2834-IKZF3 | 89 | TFORF1699-E2F5 | 130 |
| TFORF2098-ZKSCAN1 | 7 | TFORF2808-ETV7 | 61 | TFORF2905-DACH2 | 89 | TFORF1863-POU2F2 | 130 |
| TFORF2482-MXD3 | 7 | TFORF3021-DLX4 | 61 | TFORF2988-OTX2 | 89 | TFORF2180-ZNF417 | 130 |
| TFORF2717-TFCP2 | 7 | TFORF3332-PUF60 | 61 | TFORF3136-HEY1 | 89 | TFORF2605-MYRF | 130 |
| TFORF2731-ZNF669 | 7 | TFORF3540-TAL2 | 61 | TFORF0119-ELMSAN1 | 90 | TFORF2715-ZNF713 | 130 |
| TFORF3292-MXD3 | 7 | TFORF0453-THRA | 62 | TFORF0124-OLIG1 | 90 | TFORF2783-GLIS3 | 130 |
| TFORF3403-ZNF488 | 7 | TFORF0488-ZFP90 | 62 | TFORF0437-ZSCAN31 | 90 | TFORF2785-GLIS1 | 130 |
| TFORF3429-ZNF302 | 7 | TFORF0755-ZNF319 | 62 | TFORF0513-PAX5 | 90 | TFORF2786-GLIS1 | 130 |
| TFORF3440-ING1 | 7 | TFORF0857-VSX1 | 62 | TFORF0734-ZNF488 | 90 | TFORF2941-DLX5 | 130 |
| TFORF3483-PBX4 | 7 | TFORF1045-TSC22D2 | 62 | TFORF0893-ING4 | 90 | TFORF3043-ZNF75D | 130 |
| TFORF0127-SP8 | 8 | TFORF1238-HOMEZ | 62 | TFORF1076-NFATC4 | 90 | TFORF3152-ZFP3 | 130 |
| TFORF0154-THAP7 | 8 | TFORF1295-ZFP64 | 62 | TFORF1311-RBMS1 | 90 | TFORF3253-RBPJ | 130 |
| TFORF0156-EWSR1 | 8 | TFORF1458-TFEB | 62 | TFORF1984-ZNF534 | 90 | TFORF3275-HMGA1 | 130 |
| TFORF0547-PAX9 | 8 | TFORF1761-ZNF24 | 62 | TFORF2339-ZNF19 | 90 | TFORF3333-PUF60 | 130 |
| TFORF0851-ZIC4 | 8 | TFORF1881-SOX30 | 62 | TFORF2875-ELF3 | 90 | TFORF3417-TCF7L2 | 130 |
| TFORF1224-AKNA | 8 | TFORF2077-MYB | 62 | TFORF3010-NR1I3 | 90 | TFORF0581-ZNF101 | 131 |
| TFORF2065-NKX2-5 | 8 | TFORF2270-PSAP | 62 | TFORF3078-XRCC4 | 90 | TFORF0671-ZNF821 | 131 |
| TFORF2422-RBPJL | 8 | TFORF2281-MYBBP1A | 62 | TFORF3167-ZNF512B | 90 | TFORF0701-PAXBP1 | 131 |
| TFORF2611-ZNF277 | 8 | TFORF2567-ZNF654 | 62 | TFORF3371-MYBL1 | 90 | TFORF0852-ZIC4 | 131 |
| TFORF2629-INSM2 | 8 | TFORF2656-STAT3 | 62 | TFORF3511-SOX14 | 90 | TFORF1142-HMBOX1 | 131 |
| TFORF2665-ZBED2 | 8 | TFORF3053-ZBTB44 | 62 | TFORF0976-GFI1B | 91 | TFORF1171-MLX | 131 |
| TFORF2990-NFE2L1 | 8 | TFORF0003-HIF3A | 63 | TFORF1053-HKR1 | 91 | TFORF1309-TTF1 | 131 |
| TFORF3187-RAD21 | 8 | TFORF0153-THAP6 | 63 | TFORF1266-ZNF135 | 91 | TFORF1676-RUNX1T1 | 131 |
| TFORF0204-ELK4 | 9 | TFORF0475-SP140 | 63 | TFORF1294-ZFP64 | 91 | TFORF1697-E2F6 | 131 |
| TFORF0414-CTCFL | 9 | TFORF0502-NFE4 | 63 | TFORF1879-CREBZF | 91 | TFORF1775-ZNF500 | 131 |
| TFORF0493-ZSCAN2 | 9 | TFORF1163-NFKBIL1 | 63 | TFORF0023-IRX2 | 92 | TFORF1829-DMRTA2 | 131 |
| TFORF1489-NR6A1 | 9 | TFORF1657-SPIB | 63 | TFORF0053-MAZ | 92 | TFORF1937-MEIS2 | 131 |
| TFORF1573-ZNF684 | 9 | TFORF1743-ZNF764 | 63 | TFORF0058-MAF | 92 | TFORF2174-ZNF419 | 131 |
| TFORF1677-RUNX1T1 | 9 | TFORF2229-ZNF730 | 63 | TFORF0408-CTCFL | 92 | TFORF2241-GATA3 | 131 |
| TFORF2201-POU4F2 | 9 | TFORF2357-ZNF649 | 63 | TFORF0472-ZNF221 | 92 | TFORF2371-ZNF468 | 131 |
| TFORF2756-BARHL2 | 9 | TFORF2361-ZNF641 | 63 | TFORF0878-HOXB2 | 92 | TFORF2810-ETV7 | 131 |
| TFORF2937-FOS | 9 | TFORF2817-FOXD4L3 | 63 | TFORF1343-FEZF1 | 92 | TFORF2852-HMGA2 | 131 |
| TFORF2966-NR0B1 | 9 | TFORF3121-ZNF341 | 63 | TFORF2166-GRHL3 | 92 | TFORF3133-TCF7 | 131 |
| TFORF0126-CXXC1 | 10 | TFORF3299-MEIS2 | 63 | TFORF2975-NAT10 | 92 | TFORF3531-HMGN3 | 131 |
| TFORF0532-PAX1 | 10 | TFORF3330-TBX20 | 63 | TFORF3025-MYF6 | 92 | TFORF0308-CREM | 132 |
| TFORF0981-KLF6 | 10 | TFORF0037-SP5 | 64 | TFORF3108-ASCL2 | 92 | TFORF0380-SMARCA2 | 132 |
| TFORF1105-GTF2IRD2 | 10 | TFORF0358-ZNF33A | 64 | TFORF3129-SMAD7 | 92 | TFORF0925-NR2F2 | 132 |
| TFORF1621-ZNF512 | 10 | TFORF0455-TAF4B | 64 | TFORF3220-ZNF449 | 92 | TFORF1252-TP53 | 132 |
| TFORF1741-ZNF768 | 10 | TFORF0468-KAT7 | 64 | TFORF3280-DDB2 | 92 | TFORF1506-HEY2 | 132 |
| TFORF1967-PPARD | 10 | TFORF0511-TSHZ1 | 64 | TFORF3362-MYOG | 92 | TFORF1588-ZNF773 | 132 |
| TFORF2439-YEATS4 | 10 | TFORF0609-MAFF | 64 | TFORF3509-GATAD2B | 92 | TFORF2511-PDCD2 | 132 |
| TFORF2879-ELF2 | 10 | TFORF0633-VPS72 | 64 | TFORF3541-THAP6 | 92 | TFORF2655-STAT6 | 132 |
| TFORF3347-PITX1 | 10 | TFORF0650-NR1I3 | 64 | TFORF0536-PAX2 | 93 | TFORF1063-NFATC1 | 133 |
| TFORF0543-ZFAT | 11 | TFORF0799-ATF3 | 64 | TFORF0862-NR1H4 | 93 | TFORF1143-HMBOX1 | 133 |
| TFORF0678-PRDM14 | 11 | TFORF0983-KLF5 | 64 | TFORF0941-LMO1 | 93 | TFORF1312-AEBP2 | 133 |
| TFORF1289-GMEB2 | 11 | TFORF0984-KLF5 | 64 | TFORF0955-CDIP1 | 93 | TFORF1716-LDB2 | 133 |
| TFORF1541-ZBTB49 | 11 | TFORF0987-KLF2 | 64 | TFORF1034-WT1 | 93 | TFORF2079-FLI1 | 133 |
| TFORF1727-ZNF692 | 11 | TFORF0988-KLF1 | 64 | TFORF1140-HMBOX1 | 93 | TFORF2582-NR1D1 | 133 |
| TFORF1019-SCRT1 | 12 | TFORF1371-ZNF140 | 64 | TFORF1146-HOXA4 | 93 | TFORF2828-IKZF3 | 133 |
| TFORF1335-POU3F2 | 12 | TFORF1432-NR3C1 | 64 | TFORF2167-GRHL3 | 93 | TFORF3159-SMAD2 | 133 |
| TFORF1941-MIER1 | 12 | TFORF1453-DLX2 | 64 | TFORF2306-ETV1 | 93 | TFORF3312-ELK4 | 133 |
| TFORF2334-CTNNB1 | 12 | TFORF1782-ZNF239 | 64 | TFORF2347-HOXD8 | 93 | TFORF0289-MEF2C | 134 |
| TFORF2360-ZNF641 | 12 | TFORF1893-AHRR | 64 | TFORF2702-ESRRG | 93 | TFORF0438-ZSCAN30 | 134 |
| TFORF2816-FOSB | 12 | TFORF1910-TCF7L2 | 64 | TFORF2726-ZFP1 | 93 | TFORF0571-MEOX1 | 134 |
| TFORF2821-IKZF5 | 12 | TFORF2006-CAPN15 | 64 | TFORF2806-ETV7 | 93 | TFORF0750-ZNF131 | 134 |
| TFORF3002-NFYC | 12 | TFORF2015-HMGXB4 | 64 | TFORF2862-SREBF1 | 93 | TFORF0809-SOHLH1 | 134 |
| TFORF3170-CNOT3 | 12 | TFORF2046-ARID3B | 64 | TFORF3272-PRDM4 | 93 | TFORF2125-ZNF747 | 134 |
| TFORF3395-TBX22 | 12 | TFORF2589-ZNF354A | 64 | TFORF3518-NHLH2 | 93 | TFORF2356-ZNF648 | 134 |
| TFORF3545-PIAS1 | 12 | TFORF2660-STAT1 | 64 | TFORF0401-ZNF2 | 94 | TFORF2387-MBD2 | 134 |
| TFORF0276-TCF3 | 13 | TFORF2719-ZNF79 | 64 | TFORF0406-CTCFL | 94 | TFORF2686-ZMYND11 | 134 |
| TFORF0780-GTF2H1 | 13 | TFORF2868-TADA3 | 64 | TFORF0534-ZNF584 | 94 | TFORF3351-ARNTL | 134 |
| TFORF1025-FOXI2 | 13 | TFORF2871-HMX1 | 64 | TFORF0797-ATF1 | 94 | TFORF0275-TCF3 | 135 |
| TFORF1133-CARF | 13 | TFORF2950-MAFB | 64 | TFORF1919-RUNX1 | 94 | TFORF0580-ZNF107 | 135 |
| TFORF1339-POU3F4 | 13 | TFORF3125-ZNF300 | 64 | TFORF2164-GRHL2 | 94 | TFORF0795-ATF7 | 135 |
| TFORF1416-ZNF215 | 13 | TFORF3130-IRF8 | 64 | TFORF0295-MEF2A | 95 | TFORF1038-WT1 | 135 |
| TFORF1480-TP63 | 13 | TFORF3198-OVOL2 | 64 | TFORF0445-ZNF805 | 95 | TFORF1652-SPI1 | 135 |
| TFORF2292-HES7 | 13 | TFORF3354-MXD1 | 64 | TFORF0789-DBX2 | 95 | TFORF1675-RUNX1T1 | 135 |
| TFORF2315-RXRB | 13 | TFORF3412-EGR1 | 64 | TFORF0991-KLF8 | 95 | TFORF1845-ALX4 | 135 |
| TFORF2365-ZNF398 | 13 | TFORF3444-KCNIP2 | 64 | TFORF1152-MYOCD | 95 | TFORF2779-BATF | 135 |
| TFORF2508-PML | 13 | TFORF3463-ETV7 | 64 | TFORF1179-ZNF799 | 95 | TFORF3101-PES1 | 135 |
| TFORF2574-HSF2 | 13 | TFORF3543-CSDC2 | 64 | TFORF1348-RPA2 | 95 | TFORF3305-ESR1 | 135 |
| TFORF2743-T | 13 | TFORF0208-FOXP2 | 65 | TFORF1854-ZBTB5 | 95 | TFORF0608-MAFA | 136 |
| TFORF2830-IKZF3 | 13 | TFORF0209-FOXP2 | 65 | TFORF1943-MIER1 | 95 | TFORF0721-ZNF727 | 136 |
| TFORF3160-SNAI3 | 13 | TFORF0210-FOXP2 | 65 | TFORF1954-ESR2 | 95 | TFORF1155-RERE | 136 |
| TFORF3405-KCNIP4 | 13 | TFORF0214-FOXP1 | 65 | TFORF1983-TBX5 | 95 | TFORF2901-DACH1 | 136 |
| TFORF0652-NR1I3 | 14 | TFORF0215-FOXP1 | 65 | TFORF2848-TCF21 | 95 | TFORF3157-SMARCD1 | 136 |
| TFORF1080-NOBOX | 14 | TFORF0216-FOXP1 | 65 | TFORF3300-MEIS2 | 95 | TFORF3517-SOX12 | 136 |
| TFORF1536-ZBTB44 | 14 | TFORF0220-FOXP4 | 65 | TFORF3313-ZBTB25 | 95 | TFORF0224-ZNF592 | 137 |
| TFORF1560-IL18 | 14 | TFORF0221-FOXP4 | 65 | TFORF3361-MSC | 95 | TFORF0443-ZFX | 137 |
| TFORF1584-ZNF775 | 14 | TFORF0549-PAX8 | 65 | TFORF0258-TCF4 | 96 | TFORF0457-TRIM24 | 137 |
| TFORF3406-ELOF1 | 14 | TFORF0624-NKRF | 65 | TFORF1078-NFATC4 | 96 | TFORF0803-ATF2 | 137 |
| TFORF3414-NEUROG1 | 14 | TFORF0744-PIAS2 | 65 | TFORF2213-CREB3L1 | 96 | TFORF0806-SMARCC2 | 137 |
| TFORF0599-ZNF891 | 15 | TFORF0997-ZNF302 | 65 | TFORF2275-SLC22A1 | 96 | TFORF0861-NR1H4 | 137 |
| TFORF0923-NR2F2 | 15 | TFORF1093-ZBTB8B | 65 | TFORF2533-SOX1 | 96 | TFORF0986-GZF1 | 137 |
| TFORF2108-L3MBTL4 | 15 | TFORF1365-ISL1 | 65 | TFORF2748-NKX6-3 | 96 | TFORF1064-NFATC1 | 137 |
| TFORF2698-ACTL6A | 15 | TFORF1413-ZNF561 | 65 | TFORF2934-TFEB | 96 | TFORF1095-EN1 | 137 |
| TFORF2955-HMG20B | 15 | TFORF1780-ZNF506 | 65 | TFORF3178-TFDP2 | 96 | TFORF1148-HOXA2 | 137 |
| TFORF3470-ZNF726 | 15 | TFORF2057-ARGFX | 65 | TFORF3388-YEATS4 | 96 | TFORF1321-KLF11 | 137 |
| TFORF1278-ZNF587 | 16 | TFORF2308-ETV1 | 65 | TFORF3443-SP4 | 96 | TFORF1374-ZNF143 | 137 |
| TFORF1876-CCNT1 | 16 | TFORF2309-ETV1 | 65 | TFORF0022-IRX3 | 97 | TFORF1585-ZNF772 | 137 |
| TFORF2019-MYBL2 | 16 | TFORF2310-ETV1 | 65 | TFORF0038-SP7 | 97 | TFORF1661-ARID5B | 137 |
| TFORF2634-TFDP2 | 16 | TFORF2401-NFE2L3 | 65 | TFORF0110-SMAD7 | 97 | TFORF1812-ZNF438 | 137 |
| TFORF2826-IKZF1 | 16 | TFORF3181-PATZ1 | 65 | TFORF0121-BARX2 | 97 | TFORF1873-BBX | 137 |
| TFORF3421-YBX2 | 16 | TFORF3334-SP100 | 65 | TFORF0149-THAP4 | 97 | TFORF1906-TCF7L2 | 137 |
| TFORF0021-IRX1 | 17 | TFORF1631-ZNF226 | 66 | TFORF0249-CAMTA2 | 97 | TFORF1999-TAL1 | 137 |
| TFORF0257-TCF4 | 17 | TFORF1804-ZNF347 | 66 | TFORF0256-TCF4 | 97 | TFORF2003-FOXD3 | 137 |
| TFORF1742-ZNF765 | 17 | TFORF1808-TBX18 | 66 | TFORF0342-ETF1 | 97 | TFORF2031-PRDM1 | 137 |
| TFORF2523-NRF1 | 17 | TFORF1924-ZBTB21 | 66 | TFORF0452-THRA | 97 | TFORF2139-ZNF527 | 137 |
| TFORF2710-DMRTA1 | 17 | TFORF2172-ZNF419 | 66 | TFORF0496-EGR2 | 97 | TFORF2272-PRDM5 | 137 |
| TFORF2809-ETV7 | 17 | TFORF2469-IRF8 | 66 | TFORF0526-PAX7 | 97 | TFORF2316-ETV4 | 137 |
| TFORF0838-ZSCAN18 | 18 | TFORF2495-ZBTB18 | 66 | TFORF0617-ZSCAN26 | 97 | TFORF2420-ZNF705B | 137 |
| TFORF1161-NFKBIL1 | 18 | TFORF2503-PML | 66 | TFORF0921-BSX | 97 | TFORF2445-ZIK1 | 137 |
| TFORF1488-NR6A1 | 18 | TFORF2514-PDCD2 | 66 | TFORF0937-SS18 | 97 | TFORF2536-SOX6 | 137 |
| TFORF1824-ZNF138 | 18 | TFORF2572-ZNF652 | 66 | TFORF0972-ZNF124 | 97 | TFORF2542-SOX5 | 137 |
| TFORF2406-NFE2L1 | 18 | TFORF2636-TFDP2 | 66 | TFORF1342-POU5F2 | 97 | TFORF2598-RORA | 137 |
| TFORF2919-NFIB | 18 | TFORF2649-ZNF586 | 66 | TFORF1466-HOXD12 | 97 | TFORF2609-FOXA2 | 137 |
| TFORF2980-FOXP3 | 18 | TFORF2677-GABPA | 66 | TFORF1542-ZNF286B | 97 | TFORF2744-T | 137 |
| TFORF2999-CDK2 | 18 | TFORF2703-ESRRA | 66 | TFORF1552-SP110 | 97 | TFORF2959-KAT2A | 137 |
| TFORF3169-GTF2I | 18 | TFORF2752-ZNF444 | 66 | TFORF1581-ATMIN | 97 | TFORF3037-IRF2 | 137 |
| TFORF3495-CLOCK | 18 | TFORF2804-ETV2 | 66 | TFORF1586-ZNF772 | 97 | TFORF0077-KCNIP4 | 138 |
| TFORF3510-TCF25 | 18 | TFORF2837-IKZF3 | 66 | TFORF1625-ZNF517 | 97 | TFORF0233-MNX1 | 138 |
| TFORF0898-ING1 | 19 | TFORF2943-ESRRG | 66 | TFORF1801-TBX10 | 97 | TFORF0370-NFYC | 138 |
| TFORF0989-KLF9 | 19 | TFORF2994-SKP2 | 66 | TFORF1819-ZNF138 | 97 | TFORF0595-PLAG1 | 138 |
| TFORF1385-TMF1 | 19 | TFORF3145-ZNF660 | 66 | TFORF1871-POU2F1 | 97 | TFORF0968-HAND1 | 138 |
| TFORF2507-PML | 19 | TFORF3203-ZNF526 | 66 | TFORF2105-ZBTB34 | 97 | TFORF0990-KLF8 | 138 |
| TFORF2613-ZNF276 | 19 | TFORF3419-ZNF562 | 66 | TFORF2146-ZNF529 | 97 | TFORF1067-NFATC2 | 138 |
| TFORF2993-SKP2 | 19 | TFORF3515-HDGF | 66 | TFORF2153-BCL11A | 97 | TFORF1250-ZNF133 | 138 |
| TFORF3298-EZH2 | 19 | TFORF0150-THAP5 | 67 | TFORF2173-ZNF419 | 97 | TFORF1403-ZNF566 | 138 |
| TFORF0188-DMRTC2 | 20 | TFORF0263-TCF4 | 67 | TFORF2184-ZNF415 | 97 | TFORF1484-TP63 | 138 |
| TFORF1329-TFAP2A | 20 | TFORF0312-CREM | 67 | TFORF2556-ADNP2 | 97 | TFORF1740-ZNF768 | 138 |
| TFORF1563-ZNF682 | 20 | TFORF0724-NFKB2 | 67 | TFORF2645-PAX3 | 97 | TFORF1907-TCF7L2 | 138 |
| TFORF1809-TBX19 | 20 | TFORF0959-ERF | 67 | TFORF2691-TEAD2 | 97 | TFORF2449-GBX1 | 138 |
| TFORF1978-TBX1 | 20 | TFORF1678-RUNX1T1 | 67 | TFORF2925-ZFP57 | 97 | TFORF2487-ZBTB10 | 138 |
| TFORF3015-PPARA | 20 | TFORF2910-NFIC | 67 | TFORF3041-SOX15 | 97 | TFORF2612-ZNF276 | 138 |
| TFORF0145-PHOX2B | 21 | TFORF2920-NFIB | 67 | TFORF3132-ESR2 | 97 | TFORF2797-TAF7 | 138 |
| TFORF0236-MYCN | 21 | TFORF2923-NFIA | 67 | TFORF3237-SOX10 | 97 | TFORF2944-SATB2 | 138 |
| TFORF0530-PAX6 | 21 | TFORF0078-KCNIP4 | 68 | TFORF3346-NHP2 | 97 | TFORF3090-ZNF260 | 138 |
| TFORF0673-ZNF827 | 21 | TFORF0080-KCNIP3 | 68 | TFORF3360-HOXB13 | 97 | TFORF3115-GSC | 138 |
| TFORF0739-ZNF155 | 21 | TFORF0271-TCF7 | 68 | TFORF3376-TOX | 97 | TFORF3122-SATB1 | 138 |
| TFORF1027-RARA | 21 | TFORF0379-SMARCA2 | 68 | TFORF3382-ZNF140 | 97 | TFORF3140-ZNF718 | 138 |
| TFORF2827-IKZF1 | 21 | TFORF0636-HOXC5 | 68 | TFORF3431-NR3C2 | 97 | TFORF3188-ZNF394 | 138 |
| TFORF2949-GCM2 | 21 | TFORF0802-ATF2 | 68 | TFORF3484-ZNF398 | 97 | TFORF3202-ZNF816 | 138 |
| TFORF0484-ZFP92 | 22 | TFORF0811-CDCA7L | 68 | TFORF0288-MEF2C | 98 | TFORF3223-ZMIZ2 | 138 |
| TFORF0957-ZNF497 | 22 | TFORF0858-VSX1 | 68 | TFORF0360-TUB | 98 | TFORF3263-ZNF26 | 138 |
| TFORF1314-AEBP2 | 22 | TFORF0931-MECP2 | 68 | TFORF0728-MLXIP | 98 | TFORF3269-EZH1 | 138 |
| TFORF1626-ZNF514 | 22 | TFORF1347-RPA3 | 68 | TFORF0775-ZNF92 | 98 | TFORF3276-ZBTB14 | 138 |
| TFORF1765-ZNF23 | 22 | TFORF1434-NFKBID | 68 | TFORF0814-LHX1 | 98 | TFORF3342-TP53BP2 | 138 |
| TFORF1945-MIER1 | 22 | TFORF1507-HEY1 | 68 | TFORF0815-LHX2 | 98 | TFORF3372-DAXX | 138 |
| TFORF2052-BCL6 | 22 | TFORF1549-TAZ | 68 | TFORF0816-LHX3 | 98 | TFORF3430-FXN | 138 |
| TFORF2060-NKX2-1 | 22 | TFORF1843-ALX1 | 68 | TFORF0818-LHX3 | 98 | TFORF3446-ZNF786 | 138 |
| TFORF2343-MITF | 22 | TFORF2030-TGIF2LX | 68 | TFORF0819-LHX5 | 98 | TFORF3469-ZNF438 | 138 |
| TFORF2383-MBD1 | 22 | TFORF2096-ZKSCAN1 | 68 | TFORF1459-LMX1A | 98 | TFORF0185-SHOX2 | 139 |
| TFORF2854-FEV | 22 | TFORF2120-HOPX | 68 | TFORF1460-LMX1B | 98 | TFORF0212-FOXP3 | 139 |
| TFORF2986-MECP2 | 22 | TFORF2130-PFDN5 | 68 | TFORF1461-LMX1B | 98 | TFORF0357-ZNF33A | 139 |
| TFORF3004-PCGF2 | 22 | TFORF2156-RHOXF2 | 68 | TFORF1462-LMX1B | 98 | TFORF0368-NFYC | 139 |
| TFORF3030-ZNF415 | 22 | TFORF2170-BHLHE22 | 68 | TFORF1471-TOX2 | 98 | TFORF0500-PPARGC1A | 139 |
| TFORF3059-FOSL1 | 22 | TFORF2194-TFAM | 68 | TFORF1670-TP73 | 98 | TFORF0940-ZNF639 | 139 |
| TFORF3060-BACH1 | 22 | TFORF2277-PRDM2 | 68 | TFORF1897-NR2C1 | 98 | TFORF1230-AFF3 | 139 |
| TFORF3189-ZNF558 | 22 | TFORF2413-ZNF268 | 68 | TFORF2367-MBD4 | 98 | TFORF1390-PMS1 | 139 |
| TFORF3318-TFCP2 | 22 | TFORF2939-HOXA6 | 68 | TFORF2386-MBD2 | 98 | TFORF1469-TOX3 | 139 |
| TFORF0201-RFX3 | 23 | TFORF3216-ATF2 | 68 | TFORF2465-IRF6 | 98 | TFORF1685-HNF1B | 139 |
| TFORF0277-TCF3 | 23 | TFORF3422-HES6 | 68 | TFORF2647-PAX3 | 98 | TFORF1720-DMRT2 | 139 |
| TFORF0584-SRF | 23 | TFORF3459-HMGB2 | 68 | TFORF3027-SP6 | 98 | TFORF1833-MTERF1 | 139 |
| TFORF0629-NR1I2 | 23 | TFORF0107-TRERF1 | 69 | TFORF3179-LHX9 | 98 | TFORF1859-NR4A3 | 139 |
| TFORF0913-ZNF835 | 23 | TFORF0300-CREM | 69 | TFORF3212-ZKSCAN1 | 98 | TFORF1972-TBX2 | 139 |
| TFORF1853-ZBTB1 | 23 | TFORF0305-CREM | 69 | TFORF0296-MEF2A | 99 | TFORF2259-SOX13 | 139 |
| TFORF2033-OSR1 | 23 | TFORF0315-CREM | 69 | TFORF0820-LHX6 | 99 | TFORF2638-TFDP1 | 139 |
| TFORF2577-HSF4 | 23 | TFORF0622-ZSCAN23 | 69 | TFORF0821-LHX6 | 99 | TFORF3209-ZBTB9 | 139 |
| TFORF2628-NONO | 23 | TFORF0631-TERF1 | 69 | TFORF0822-LHX6 | 99 | TFORF3217-ZNF227 | 139 |
| TFORF3064-TERF1 | 23 | TFORF1107-ZNF37A | 69 | TFORF0824-LHX8 | 99 | TFORF3268-ZNF34 | 139 |
| TFORF0052-MAZ | 24 | TFORF1430-NFKBIB | 69 | TFORF0825-LHX8 | 99 | TFORF3453-PREB | 139 |
| TFORF0268-TCF4 | 24 | TFORF1472-TOX2 | 69 | TFORF1825-ARNT | 99 | TFORF3524-MAEL | 139 |
| TFORF0688-PRDM10 | 24 | TFORF1520-NR2E1 | 69 | TFORF2058-NKX2-2 | 99 | TFORF0197-RFX1 | 140 |
| TFORF0929-ATOH8 | 24 | TFORF1820-ZNF138 | 69 | TFORF2625-CRTC3 | 99 | TFORF0767-ZNF550 | 140 |
| TFORF1075-NFATC4 | 24 | TFORF1832-MTERF1 | 69 | TFORF2873-ELF1 | 99 | TFORF0846-YBX1 | 140 |
| TFORF1251-ZNF133 | 24 | TFORF1965-PPARD | 69 | TFORF3233-JUNB | 99 | TFORF1116-RCOR1 | 140 |
| TFORF1267-ZNF135 | 24 | TFORF2021-NPAS4 | 69 | TFORF0674-ZNF829 | 100 | TFORF1400-ZNF567 | 140 |
| TFORF1336-POU3F1 | 24 | TFORF2023-NPAS3 | 69 | TFORF1012-ZNF546 | 100 | TFORF1687-HNF1B | 140 |
| TFORF1570-ZNF680 | 24 | TFORF2049-PURA | 69 | TFORF1517-FOXD4L4 | 100 | TFORF1694-E4F1 | 140 |
| TFORF1702-E2F4 | 24 | TFORF2191-RAX2 | 69 | TFORF1641-PSIP1 | 100 | TFORF1732-HDX | 140 |
| TFORF2053-BCL6 | 24 | TFORF2208-CREB3L2 | 69 | TFORF2375-MBD1 | 100 | TFORF1796-WDHD1 | 140 |
| TFORF2643-PAX3 | 24 | TFORF2314-RXRB | 69 | TFORF2381-MBD1 | 100 | TFORF2163-GRHL2 | 140 |
| TFORF2685-ZMYND11 | 24 | TFORF2359-ZNF644 | 69 | TFORF2530-SOX8 | 100 | TFORF2858-ID3 | 140 |
| TFORF3013-MEF2A | 24 | TFORF2384-MBD3 | 69 | TFORF2671-NEUROD4 | 100 | TFORF2926-BCL6B | 140 |
| TFORF3092-NPAS1 | 24 | TFORF2557-ZNF69 | 69 | TFORF2945-NEUROD1 | 100 | TFORF2962-NR2C2 | 140 |
| TFORF3232-JUNB | 24 | TFORF2687-ZMYND11 | 69 | TFORF3260-ZNF524 | 100 | TFORF2979-NR0B2 | 140 |
| TFORF0103-LEUTX | 25 | TFORF2704-ESRRA | 69 | TFORF0010-ZNF879 | 101 | TFORF3088-E4F1 | 140 |
| TFORF0400-ZNF2 | 25 | TFORF2860-ID1 | 69 | TFORF0391-FERD3L | 101 | TFORF3097-ZNF76 | 140 |
| TFORF0899-ING1 | 25 | TFORF2903-DACH1 | 69 | TFORF0577-GTF2A1L | 101 | TFORF3124-SMAD3 | 140 |
| TFORF1240-LBX2 | 25 | TFORF3006-ZBTB12 | 69 | TFORF0616-ZSCAN26 | 101 | TFORF3204-LITAF | 140 |
| TFORF1774-ZNF500 | 25 | TFORF3035-NFKBIB | 69 | TFORF0639-HOXC6 | 101 | TFORF3227-RPA2 | 140 |
| TFORF1946-MIER1 | 25 | TFORF3049-CDK1 | 69 | TFORF1150-CDK7 | 101 | TFORF3286-NR2F6 | 140 |
| TFORF2221-YAP1 | 25 | TFORF3051-CDK1 | 69 | TFORF1223-ZNF483 | 101 | TFORF3516-RNF138 | 140 |
| TFORF2264-ZNF286A | 25 | TFORF3238-GTF2A1 | 69 | TFORF1502-GABPB1 | 101 | TFORF0157-EWSR1 | 141 |
| TFORF2368-MBD4 | 25 | TFORF3244-ZNF483 | 69 | TFORF2322-ZNF736 | 101 | TFORF0175-RELA | 141 |
| TFORF2502-PML | 25 | TFORF3316-MED21 | 69 | TFORF2355-PROP1 | 101 | TFORF0245-CAMTA2 | 141 |
| TFORF2578-MEIS3 | 25 | TFORF3373-ZNF101 | 69 | TFORF2684-ZMYND11 | 101 | TFORF0369-NFYC | 141 |
| TFORF2690-TEAD2 | 25 | TFORF3396-TARBP2 | 69 | TFORF2989-SLC45A2 | 101 | TFORF0412-CTCFL | 141 |
| TFORF3036-ATF4 | 25 | TFORF3410-ZKSCAN4 | 69 | TFORF3079-ZNF846 | 101 | TFORF0554-ZNF610 | 141 |
| TFORF0520-PAX5 | 26 | TFORF0018-IRX4 | 70 | TFORF0164-SIX4 | 102 | TFORF0585-ZNF329 | 141 |
| TFORF0535-ZNF584 | 26 | TFORF0099-HLF | 70 | TFORF0172-ONECUT2 | 102 | TFORF0749-ZNF131 | 141 |
| TFORF1465-VAV1 | 26 | TFORF0111-SMAD7 | 70 | TFORF0686-PRDM10 | 102 | TFORF0786-FOXN1 | 141 |
| TFORF1658-LEF1 | 26 | TFORF0148-THAP4 | 70 | TFORF0745-PIAS2 | 102 | TFORF0884-HOXB4 | 141 |
| TFORF1856-NR4A2 | 26 | TFORF0178-HDGF | 70 | TFORF1334-POU3F3 | 102 | TFORF0902-PTF1A | 141 |
| TFORF2462-IRF7 | 26 | TFORF0205-DBP | 70 | TFORF1659-LEF1 | 102 | TFORF0946-LMO3 | 141 |
| TFORF2822-IKZF4 | 26 | TFORF0211-FOXP3 | 70 | TFORF1799-USF2 | 102 | TFORF1007-ZNF544 | 141 |
| TFORF2911-NFIC | 26 | TFORF0225-ZNF593 | 70 | TFORF2376-MBD1 | 102 | TFORF1011-ZNF546 | 141 |
| TFORF3234-VSX1 | 26 | TFORF0280-IGFBP1 | 70 | TFORF2499-ASCL5 | 102 | TFORF1132-BNC1 | 141 |
| TFORF0151-THAP5 | 27 | TFORF0307-CREM | 70 | TFORF3100-ZNF212 | 102 | TFORF1315-ZNF813 | 141 |
| TFORF1032-RARG | 27 | TFORF0310-CREM | 70 | TFORF3106-CCNH | 102 | TFORF1340-POU5F1 | 141 |
| TFORF1698-E2F6 | 27 | TFORF0314-CREM | 70 | TFORF0895-ING4 | 103 | TFORF1361-SP100 | 141 |
| TFORF2391-ZNF461 | 27 | TFORF0351-FOXL2 | 70 | TFORF1147-HOXA3 | 103 | TFORF1405-ZNF566 | 141 |
| TFORF2736-PLK4 | 27 | TFORF0539-ZNF589 | 70 | TFORF1483-TP63 | 103 | TFORF1523-NFRKB | 141 |
| TFORF3102-ZNF689 | 27 | TFORF0592-FOXO4 | 70 | TFORF1638-BNIP3 | 103 | TFORF1569-ZNF680 | 141 |
| TFORF3338-HDAC1 | 27 | TFORF0615-ZSCAN26 | 70 | TFORF2276-SLC22A1 | 103 | TFORF1579-ZNF778 | 141 |
| TFORF0014-ZNF707 | 28 | TFORF0653-NR1I3 | 70 | TFORF2332-ZNF492 | 103 | TFORF1601-ZNF195 | 141 |
| TFORF0075-KCNIP4 | 28 | TFORF0717-TEF | 70 | TFORF2884-FIGLA | 103 | TFORF1617-NF1 | 141 |
| TFORF0115-SMAD3 | 28 | TFORF0733-PROX2 | 70 | TFORF3083-FOSL2 | 103 | TFORF1647-ZNF358 | 141 |
| TFORF0509-TSHZ2 | 28 | TFORF0792-TARBP2 | 70 | TFORF3548-THAP10 | 103 | TFORF1648-ZNF429 | 141 |
| TFORF0903-SPDEF | 28 | TFORF0875-GLMP | 70 | TFORF0564-ZNF619 | 104 | TFORF1674-RUNX1T1 | 141 |
| TFORF0966-ZNF493 | 28 | TFORF0876-GLMP | 70 | TFORF1216-NHLH1 | 104 | TFORF1755-ZNF182 | 141 |
| TFORF0993-KLF8 | 28 | TFORF0932-ZNF80 | 70 | TFORF1748-ZNF763 | 104 | TFORF1766-ZNF23 | 141 |
| TFORF1018-ZNF548 | 28 | TFORF0960-ERG | 70 | TFORF1870-POU2F1 | 104 | TFORF1793-CNBP | 141 |
| TFORF1074-NFATC4 | 28 | TFORF1052-HKR1 | 70 | TFORF1994-ZHX1 | 104 | TFORF1949-MIER2 | 141 |
| TFORF1275-TPRX1 | 28 | TFORF1091-ZBTB8A | 70 | TFORF2093-ZFP14 | 104 | TFORF1970-ZNF233 | 141 |
| TFORF1360-SP100 | 28 | TFORF1144-HOXA7 | 70 | TFORF2479-MESP2 | 104 | TFORF2040-TP53BP2 | 141 |
| TFORF1373-ZNF143 | 28 | TFORF1200-SETDB1 | 70 | TFORF0035-SP3 | 105 | TFORF2070-MYB | 141 |
| TFORF1380-ZNF787 | 28 | TFORF1214-FOXH1 | 70 | TFORF0338-MECOM | 105 | TFORF2175-ZNF419 | 141 |
| TFORF1422-ZNF211 | 28 | TFORF1222-ZNF483 | 70 | TFORF0881-HOXB3 | 105 | TFORF2209-CREB3L3 | 141 |
| TFORF1518-FOXD4L5 | 28 | TFORF1259-TP53 | 70 | TFORF1058-NFATC1 | 105 | TFORF2302-CIITA | 141 |
| TFORF1989-ZNF530 | 28 | TFORF1383-ZNF788 | 70 | TFORF1124-E2F7 | 105 | TFORF2441-FUS | 141 |
| TFORF2160-TXK | 28 | TFORF1445-GTF2H3 | 70 | TFORF2764-ZKSCAN5 | 105 | TFORF2488-ZBTB10 | 141 |
| TFORF2189-ZNF410 | 28 | TFORF1671-TP73 | 70 | TFORF3401-GTF2B | 105 | TFORF2519-ZFP37 | 141 |
| TFORF2227-ZNF343 | 28 | TFORF1822-ZNF138 | 70 | TFORF3402-HSFY2 | 105 | TFORF2538-SOX4 | 141 |
| TFORF2481-MXD4 | 28 | TFORF1847-ETS1 | 70 | TFORF0130-HMGB3 | 106 | TFORF2627-NONO | 141 |
| TFORF2494-ZBTB18 | 28 | TFORF1851-ZBTB3 | 70 | TFORF0770-MSX2 | 106 | TFORF2644-PAX3 | 141 |
| TFORF2573-HSF2 | 28 | TFORF1861-DRGX | 70 | TFORF1167-EBF3 | 106 | TFORF2768-KDM5D | 141 |
| TFORF2692-TEAD1 | 28 | TFORF1882-SOX30 | 70 | TFORF1168-EBF2 | 106 | TFORF2833-IKZF3 | 141 |
| TFORF2711-ZNF710 | 28 | TFORF1900-PATZ1 | 70 | TFORF1695-ZNF114 | 106 | TFORF2845-TCF24 | 141 |
| TFORF3086-STAT1 | 28 | TFORF1902-TCF7L2 | 70 | TFORF2224-YAP1 | 106 | TFORF2880-ELF2 | 141 |
| TFORF3218-FOXN3 | 28 | TFORF2305-ETV1 | 70 | TFORF2659-STAT2 | 106 | TFORF2888-BRPF1 | 141 |
| TFORF3262-CEBPE | 28 | TFORF2321-TAF1A | 70 | TFORF3022-RXRG | 106 | TFORF2936-FOS | 141 |
| TFORF3285-FEZF1 | 28 | TFORF2337-ZNF18 | 70 | TFORF3058-EBF1 | 106 | TFORF2973-PPARD | 141 |
| TFORF3407-ZNF397 | 28 | TFORF2450-MLLT10 | 70 | TFORF3073-LMO2 | 106 | TFORF3061-SNAPC3 | 141 |
| TFORF3439-FOXI1 | 28 | TFORF2464-IRF6 | 70 | TFORF0088-ZNF408 | 107 | TFORF3081-ELK3 | 141 |
| TFORF3468-PKNOX1 | 28 | TFORF2555-SUZ12 | 70 | TFORF0104-SMAD9 | 107 | TFORF3098-ZNF467 | 141 |
| TFORF0020-IRX6 | 29 | TFORF2646-PAX3 | 70 | TFORF0123-OLIG2 | 107 | TFORF3190-ZNF554 | 141 |
| TFORF0343-ETF1 | 29 | TFORF2663-STOX1 | 70 | TFORF0521-PAX5 | 107 | TFORF3197-STAT5A | 141 |
| TFORF0644-NR1I3 | 29 | TFORF2670-NEUROD2 | 70 | TFORF0576-HOXA10 | 107 | TFORF3295-SPDEF | 141 |
| TFORF0645-NR1I3 | 29 | TFORF2672-CHURC1 | 70 | TFORF1166-EBF4 | 107 | TFORF3369-BLZF1 | 141 |
| TFORF0697-BATF2 | 29 | TFORF2673-CHURC1 | 70 | TFORF1433-NR3C1 | 107 | TFORF3478-FOXD4 | 141 |
| TFORF0810-SOHLH1 | 29 | TFORF2694-ELF5 | 70 | TFORF1522-NR2E3 | 107 | TFORF3507-NOCT | 141 |
| TFORF1037-WT1 | 29 | TFORF2695-ELF5 | 70 | TFORF2938-SOX5 | 107 | TFORF0114-SMAD3 | 142 |
| TFORF1663-ARID5A | 29 | TFORF2701-ESRRG | 70 | TFORF3074-LMO2 | 107 | TFORF0352-FOXL1 | 142 |
| TFORF2236-GATA4 | 29 | TFORF2802-ETV2 | 70 | TFORF3195-ONECUT1 | 107 | TFORF0667-TGFB1I1 | 142 |
| TFORF2267-ARNT2 | 29 | TFORF2844-HNRNPK | 70 | TFORF3475-IL18 | 107 | TFORF0793-ATF5 | 142 |
| TFORF2759-L3MBTL1 | 29 | TFORF2853-HMGA2 | 70 | TFORF0869-NR1H3 | 108 | TFORF1077-NFATC4 | 142 |
| TFORF2867-TADA3 | 29 | TFORF3257-ZNF800 | 70 | TFORF0891-ING4 | 108 | TFORF1098-UBP1 | 142 |
| TFORF2906-DACH2 | 29 | TFORF3258-TAF12 | 70 | TFORF1718-LDB1 | 108 | TFORF1264-ZNF135 | 142 |
| TFORF3024-HEY2 | 29 | TFORF3447-MYCBP | 70 | TFORF1956-ESR2 | 108 | TFORF1567-ZNF683 | 142 |
| TFORF3057-YAF2 | 29 | TFORF3460-PSMB1 | 70 | TFORF2583-MIXL1 | 108 | TFORF1839-EZH2 | 142 |
| TFORF3392-CNBP | 29 | TFORF3513-GLMP | 70 | TFORF3546-TSC22D3 | 108 | TFORF2133-MNAT1 | 142 |
| TFORF3498-APEX1 | 29 | TFORF0189-RFX8 | 71 | TFORF0716-ZNF621 | 109 | TFORF2320-TAF1A | 142 |
| TFORF0170-ZNF587B | 30 | TFORF0203-ELK1 | 71 | TFORF0979-KLF7 | 109 | TFORF2460-IRF7 | 142 |
| TFORF1285-ZNF257 | 30 | TFORF2674-CHURC1 | 71 | TFORF1328-TFAP2A | 109 | TFORF2461-IRF7 | 142 |
| TFORF1908-TCF7L2 | 30 | TFORF3490-TCF23 | 71 | TFORF1331-TFAP2B | 109 | TFORF3139-PPARG | 142 |
| TFORF2369-MBD4 | 30 | TFORF0439-ZSCAN30 | 72 | TFORF1332-TFAP2E | 109 | TFORF0294-MEF2A | 143 |
| TFORF2620-CRX | 30 | TFORF0506-CDK2 | 72 | TFORF1635-HOXC11 | 109 | TFORF0980-KLF7 | 143 |
| TFORF2697-ZFP42 | 30 | TFORF0541-GCFC2 | 72 | TFORF1644-ZNF425 | 109 | TFORF1598-ZNF195 | 143 |
| TFORF3114-BHLHA15 | 30 | TFORF0834-HSFY1 | 72 | TFORF1939-MEIS1 | 109 | TFORF2027-NPAS2 | 143 |
| TFORF3158-SMARCB1 | 30 | TFORF0870-NR1H3 | 72 | TFORF2064-NKX2-5 | 109 | TFORF2051-ATF6B | 143 |
| TFORF3284-PBX3 | 30 | TFORF1029-RARB | 72 | TFORF2161-GRHL1 | 109 | TFORF2751-ZNF444 | 143 |
| TFORF0051-MAZ | 31 | TFORF1194-ZNF575 | 72 | TFORF2165-GRHL3 | 109 | TFORF2898-MTA3 | 143 |
| TFORF0059-CTBP1 | 31 | TFORF1258-TP53 | 72 | TFORF2168-GRHL3 | 109 | TFORF0101-FOXC2 | 144 |
| TFORF0167-SIX1 | 31 | TFORF1449-DLX4 | 72 | TFORF2193-TFAM | 109 | TFORF0105-TRERF1 | 144 |
| TFORF0887-ZNF384 | 31 | TFORF1933-MEIS2 | 72 | TFORF2218-YAP1 | 109 | TFORF0273-TCF3 | 144 |
| TFORF1110-ISX | 31 | TFORF1957-ESR2 | 72 | TFORF2266-ANHX | 109 | TFORF0482-DMRTB1 | 144 |
| TFORF1112-RCOR3 | 31 | TFORF1966-PPARD | 72 | TFORF2700-ESRRG | 109 | TFORF1005-POU1F1 | 144 |
| TFORF1172-ZNF157 | 31 | TFORF2177-ZNF419 | 72 | TFORF2755-ZNF440 | 109 | TFORF1048-PLAGL2 | 144 |
| TFORF1304-ZMIZ2 | 31 | TFORF2286-HES4 | 72 | TFORF2795-ZNF283 | 109 | TFORF1272-DUXA | 144 |
| TFORF1503-GABPB1 | 31 | TFORF2580-MEIS3 | 72 | TFORF2839-IKZF3 | 109 | TFORF1355-ILF3 | 144 |
| TFORF1568-ZNF683 | 31 | TFORF0047-MAX | 73 | TFORF2932-TFAP2A | 109 | TFORF1537-ZBTB45 | 144 |
| TFORF1771-TBP | 31 | TFORF0100-NOV | 73 | TFORF3127-NFIL3 | 109 | TFORF1815-ZNF433 | 144 |
| TFORF2395-ZNF396 | 31 | TFORF0146-THAP1 | 73 | TFORF0008-ZNF709 | 110 | TFORF1931-XRCC6 | 144 |
| TFORF2836-IKZF3 | 31 | TFORF0207-FOXP2 | 73 | TFORF0009-ZNF708 | 110 | TFORF2094-DEK | 144 |
| TFORF3355-FOXN2 | 31 | TFORF0494-ZSCAN2 | 73 | TFORF0030-ZNF43 | 110 | TFORF2199-RXRA | 144 |
| TFORF0116-FOXM1 | 32 | TFORF0642-NR1I3 | 73 | TFORF0106-TRERF1 | 110 | TFORF2723-ZNF76 | 144 |
| TFORF0583-ZNF324 | 32 | TFORF0944-LMO3 | 73 | TFORF0259-TCF4 | 110 | TFORF2777-KDM5C | 144 |
| TFORF1684-HNF1B | 32 | TFORF1028-RARA | 73 | TFORF0544-ZFAT | 110 | TFORF2840-IKZF3 | 144 |
| TFORF1688-HNF1A | 32 | TFORF1149-HOXA1 | 73 | TFORF0725-NFKB2 | 110 | TFORF2951-TWIST2 | 144 |
| TFORF1689-HNF1A | 32 | TFORF1226-DUX4 | 73 | TFORF1301-HMG20A | 110 | TFORF3448-TP63 | 144 |
| TFORF1865-POU2F2 | 32 | TFORF1963-CUX1 | 73 | TFORF1424-ZNF211 | 110 | TFORF0029-ZNF43 | 145 |
| TFORF2110-NR4A1 | 32 | TFORF2485-MXD1 | 73 | TFORF1490-NR6A1 | 110 | TFORF0113-SMAD3 | 145 |
| TFORF0086-LIN28B | 33 | TFORF2552-BID | 73 | TFORF1499-RBL1 | 110 | TFORF0287-MEF2C | 145 |
| TFORF0299-CREM | 33 | TFORF0297-CREM | 74 | TFORF1500-RBL2 | 110 | TFORF0620-ZSCAN21 | 145 |
| TFORF0638-HOXC6 | 33 | TFORF0364-TCEB1 | 74 | TFORF1521-NR2E3 | 110 | TFORF0687-PRDM10 | 145 |
| TFORF0839-HSFY2 | 33 | TFORF0522-PAX5 | 74 | TFORF1580-ZNF778 | 110 | TFORF0758-OVOL3 | 145 |
| TFORF1772-TBP | 33 | TFORF0604-PUF60 | 74 | TFORF1622-ZNF513 | 110 | TFORF0856-VSX1 | 145 |
| TFORF2455-IRF3 | 33 | TFORF0830-SLC45A2 | 74 | TFORF1789-ZNF232 | 110 | TFORF0934-ZNF85 | 145 |
| TFORF3226-FGF3 | 33 | TFORF1170-MLX | 74 | TFORF1971-YY2 | 110 | TFORF1055-PLAGL1 | 145 |
| TFORF0587-NMI | 34 | TFORF1544-ZNF160 | 74 | TFORF2055-CDX1 | 110 | TFORF1066-NFATC2 | 145 |
| TFORF0651-NR1I3 | 34 | TFORF1630-ZNF226 | 74 | TFORF2127-ZNF746 | 110 | TFORF1090-TULP1 | 145 |
| TFORF1096-EN2 | 34 | TFORF1668-TP73 | 74 | TFORF2738-PLK4 | 110 | TFORF1587-ZNF772 | 145 |
| TFORF1519-FOXD4L6 | 34 | TFORF1756-ZNF189 | 74 | TFORF2811-ETV7 | 110 | TFORF1593-ZNF197 | 145 |
| TFORF1557-YAF2 | 35 | TFORF2141-ZNF525 | 74 | TFORF2823-IKZF1 | 110 | TFORF1650-ZSCAN5A | 145 |
| TFORF1717-LDB1 | 35 | TFORF2171-BHLHE23 | 74 | TFORF2838-IKZF3 | 110 | TFORF1667-TP73 | 145 |
| TFORF2231-EHF | 35 | TFORF2216-NOTO | 74 | TFORF2895-MTA1 | 110 | TFORF1705-E2F2 | 145 |
| TFORF3279-LMO1 | 35 | TFORF2291-HES6 | 74 | TFORF2907-DACH2 | 110 | TFORF1726-ZNF692 | 145 |
| TFORF0462-CREB5 | 36 | TFORF2662-STOX1 | 74 | TFORF2963-NR2C2 | 110 | TFORF1763-ZNF26 | 145 |
| TFORF0746-PIAS3 | 36 | TFORF2792-ZNF282 | 74 | TFORF3008-CDX2 | 110 | TFORF1926-ZBTB25 | 145 |
| TFORF1450-DLX6 | 36 | TFORF3063-FOXP1 | 74 | TFORF3291-CDK9 | 110 | TFORF1927-ZNF75D | 145 |
| TFORF1660-LEF1 | 36 | TFORF0133-ZNF780A | 75 | TFORF3320-HDX | 110 | TFORF1982-TBX5 | 145 |
| TFORF2273-PRDM6 | 36 | TFORF0177-HDGF | 75 | TFORF3400-GMEB1 | 110 | TFORF2119-HOPX | 145 |
| TFORF2397-ZNF397 | 36 | TFORF0451-THRA | 75 | TFORF3462-BRPF1 | 110 | TFORF2182-ZNF415 | 145 |
| TFORF3001-DR1 | 36 | TFORF0855-VSX1 | 75 | TFORF3477-POU5F1 | 110 | TFORF2262-ZFP28 | 145 |
| TFORF0235-MYCN | 37 | TFORF0867-NR1H2 | 75 | TFORF0247-CAMTA2 | 111 | TFORF2307-ETV1 | 145 |
| TFORF0597-SMARCB1 | 37 | TFORF0889-ZNF384 | 75 | TFORF0440-ZFY | 111 | TFORF2410-ZNF705G | 145 |
| TFORF0628-NR1I2 | 37 | TFORF0890-ING4 | 75 | TFORF0456-HOXD9 | 111 | TFORF2458-IRF3 | 145 |
| TFORF1042-TSC22D1 | 37 | TFORF0936-SS18 | 75 | TFORF0465-NKX1-1 | 111 | TFORF2648-PAX3 | 145 |
| TFORF1125-ENO1 | 37 | TFORF1218-PRRX1 | 75 | TFORF1060-NFATC1 | 111 | TFORF2667-ZBED4 | 145 |
| TFORF1169-EBF1 | 37 | TFORF1262-TP53 | 75 | TFORF1604-HINFP | 111 | TFORF2727-ZFP1 | 145 |
| TFORF1713-LDB2 | 37 | TFORF1327-KLF14 | 75 | TFORF1785-ZNF235 | 111 | TFORF2761-ZKSCAN3 | 145 |
| TFORF3180-RARG | 37 | TFORF1452-DLX1 | 75 | TFORF3093-ZNF574 | 111 | TFORF2787-DCP1A | 145 |
| TFORF3247-LEF1 | 37 | TFORF1527-MXI1 | 75 | TFORF3290-CTCF | 111 | TFORF2931-LHX4 | 145 |
| TFORF3514-PURB | 37 | TFORF1548-TAZ | 75 | TFORF3427-NFIB | 111 | TFORF3047-ZNF266 | 145 |
| TFORF0495-EGR2 | 38 | TFORF1709-ZFP41 | 75 | TFORF3547-CBFB | 111 | TFORF3103-HOXA1 | 145 |
| TFORF0832-GTF2F1 | 38 | TFORF1762-ZNF24 | 75 | TFORF0405-CTCFL | 112 | TFORF3214-ZNF484 | 145 |
| TFORF0880-HOXB3 | 38 | TFORF1996-ZNF248 | 75 | TFORF0924-NR2F2 | 112 | TFORF0142-FXN | 146 |
| TFORF0927-DDB2 | 38 | TFORF2296-TAF11 | 75 | TFORF1426-FOXK2 | 112 | TFORF0262-TCF4 | 146 |
| TFORF1935-MEIS2 | 38 | TFORF3239-HOXB5 | 75 | TFORF1468-HOXD11 | 112 | TFORF0303-CREM | 146 |
| TFORF2477-OXSR1 | 38 | TFORF3389-ZNF580 | 75 | TFORF1475-LIN54 | 112 | TFORF0550-PAX8 | 146 |
| TFORF2492-ZBTB17 | 38 | TFORF0034-SP2 | 76 | TFORF1505-ZNF696 | 112 | TFORF0783-SEBOX | 146 |
| TFORF0527-PAX7 | 39 | TFORF0060-CTBP2 | 76 | TFORF1758-ZNF189 | 112 | TFORF0828-ZNF585B | 146 |
| TFORF1823-ZNF138 | 39 | TFORF0424-PBX3 | 76 | TFORF1864-POU2F2 | 112 | TFORF1059-NFATC1 | 146 |
| TFORF2050-ATF6B | 39 | TFORF0528-PAX6 | 76 | TFORF1916-RUNX2 | 112 | TFORF1417-ZNF214 | 146 |
| TFORF2683-ZMYND11 | 39 | TFORF0829-SLC45A2 | 76 | TFORF2149-ZNF250 | 112 | TFORF1642-ZNF426 | 146 |
| TFORF0176-RELA | 40 | TFORF1035-WT1 | 76 | TFORF2200-POU4F1 | 112 | TFORF1656-SPIB | 146 |
| TFORF0469-KAT7 | 40 | TFORF1320-KLF11 | 76 | TFORF2274-SLC22A1 | 112 | TFORF2128-ZNF740 | 146 |
| TFORF0714-ZNF620 | 40 | TFORF1591-CTCF | 76 | TFORF2541-SOX5 | 112 | TFORF2610-FOXA2 | 146 |
| TFORF1087-TULP3 | 40 | TFORF1600-ZNF195 | 76 | TFORF3056-THRB | 112 | TFORF2721-ZNF79 | 146 |
| TFORF1196-ZNF577 | 40 | TFORF1929-XRCC4 | 76 | TFORF3210-VSX2 | 112 | TFORF2983-SUZ12 | 146 |
| TFORF1324-KLF17 | 40 | TFORF2176-ZNF419 | 76 | TFORF3236-E2F8 | 112 | TFORF3149-ZNF254 | 146 |
| TFORF1381-ZNF789 | 40 | TFORF2297-TAF10 | 76 | TFORF3493-ZNF791 | 112 | TFORF3297-HIF1A | 146 |
| TFORF1818-UHRF1 | 40 | TFORF2483-CBX2 | 76 | TFORF0002-HIF3A | 113 | TFORF0168-SIX2 | 147 |
| TFORF1842-YWHAZ | 40 | TFORF2835-IKZF3 | 76 | TFORF0190-RFX4 | 113 | TFORF0226-ZNF593 | 147 |
| TFORF1961-CUX1 | 40 | TFORF2935-MAFK | 76 | TFORF0318-ZNF117 | 113 | TFORF0347-FOXB1 | 147 |
| TFORF2261-DMAP1 | 40 | TFORF3050-CDK1 | 76 | TFORF0497-EGR3 | 113 | TFORF0427-PBX1 | 147 |
| TFORF2378-MBD1 | 40 | TFORF3148-NFE2 | 76 | TFORF0684-PRDM13 | 113 | TFORF0572-MEOX1 | 147 |
| TFORF2463-IRF7 | 40 | TFORF3228-MAX | 76 | TFORF0866-GTF2I | 113 | TFORF0573-MEOX2 | 147 |
| TFORF2614-ZNF275 | 40 | TFORF3383-NFKBID | 76 | TFORF0896-ZNF383 | 113 | TFORF0593-UNCX | 147 |
| TFORF2681-ZMYND11 | 40 | TFORF0144-PHOX2A | 77 | TFORF1006-ZNF564 | 113 | TFORF0715-ZNF620 | 147 |
| TFORF2971-NR1H4 | 40 | TFORF0194-RFX6 | 77 | TFORF1062-NFATC1 | 113 | TFORF0778-SHOX | 147 |
| TFORF3264-ARNTL2 | 40 | TFORF0319-MSLN | 77 | TFORF1572-ZNF687 | 113 | TFORF0945-LMO3 | 147 |
| TFORF0061-CTBP2 | 41 | TFORF0723-NFKB1 | 77 | TFORF1737-ZBTB7B | 113 | TFORF1008-ZNF544 | 147 |
| TFORF0097-PSMD14 | 41 | TFORF0747-PIAS1 | 77 | TFORF1768-ZNF28 | 113 | TFORF1220-HELT | 147 |
| TFORF0286-MEF2C | 41 | TFORF0873-HOXB8 | 77 | TFORF2026-NPAS3 | 113 | TFORF1429-NR3C2 | 147 |
| TFORF0422-PBX2 | 41 | TFORF0906-DNAJC2 | 77 | TFORF2117-TBPL2 | 113 | TFORF1830-ZNF717 | 147 |
| TFORF0777-SHOX | 41 | TFORF0954-CDIP1 | 77 | TFORF2282-MYBBP1A | 113 | TFORF1969-ZNF233 | 147 |
| TFORF2054-BCL3 | 41 | TFORF1254-TP53 | 77 | TFORF2364-CREB1 | 113 | TFORF2300-HIC1 | 147 |
| TFORF2129-ZNF749 | 41 | TFORF1274-PKNOX1 | 77 | TFORF2471-HDAC5 | 113 | TFORF2348-HOXD8 | 147 |
| TFORF2210-CREB3L3 | 41 | TFORF1456-TFEC | 77 | TFORF2708-ARX | 113 | TFORF2447-ZIK1 | 147 |
| TFORF2243-LITAF | 41 | TFORF1476-VAX1 | 77 | TFORF2774-KDM5C | 113 | TFORF2484-CBX2 | 147 |
| TFORF2570-TGIF1 | 41 | TFORF1558-YAF2 | 77 | TFORF3166-ZNF512B | 113 | TFORF2496-ASCL3 | 147 |
| TFORF2588-CERS5 | 41 | TFORF1673-TP73 | 77 | TFORF3219-ZNF677 | 113 | TFORF2497-ASCL1 | 147 |
| TFORF2767-ZKSCAN7 | 41 | TFORF1733-ZNF169 | 77 | TFORF3256-ZNF189 | 113 | TFORF2498-ASCL4 | 147 |
| TFORF3042-TAF9 | 41 | TFORF1795-CNBP | 77 | TFORF3340-HDAC3 | 113 | TFORF2534-SOX6 | 147 |
| TFORF3118-ZNF581 | 41 | TFORF1841-HBP1 | 77 | TFORF0193-RFX5 | 114 | TFORF2539-SOX5 | 147 |
| TFORF3537-TSC22D4 | 41 | TFORF2000-MYEF2 | 77 | TFORF0317-MEF2D | 114 | TFORF2596-RORB | 147 |
| TFORF0411-CTCFL | 42 | TFORF2018-PITX3 | 77 | TFORF0498-EGR3 | 114 | TFORF2693-TEAD4 | 147 |
| TFORF1410-RNF2 | 42 | TFORF2642-PAX3 | 77 | TFORF1023-FOXI1 | 114 | TFORF2863-SREBF1 | 147 |
| TFORF1862-OTP | 42 | TFORF2894-MTA1 | 77 | TFORF1399-ZNF568 | 114 | TFORF2958-SP1 | 147 |
| TFORF2020-MYBL2 | 42 | TFORF2972-NR1H4 | 77 | TFORF1524-NFRKB | 114 | TFORF3062-ZNF549 | 147 |
| TFORF2872-HMX3 | 42 | TFORF3105-CCNH | 77 | TFORF1636-HOXC13 | 114 | TFORF3250-ZKSCAN8 | 147 |
| TFORF0129-SP9 | 43 | TFORF3107-RARA | 77 | TFORF1686-HNF1B | 114 | TFORF3367-ZNF281 | 147 |
| TFORF0689-HNF4G | 43 | TFORF3128-IRF5 | 77 | TFORF1704-E2F3 | 114 | TFORF3435-SUPT5H | 147 |
| TFORF0690-HNF4G | 43 | TFORF3191-ZNF436 | 77 | TFORF1848-ETS1 | 114 | TFORF3472-NFATC3 | 147 |
| TFORF0691-HNF4A | 43 | TFORF3370-MYBL1 | 77 | TFORF2043-ARID3A | 114 | TFORF3479-ZNF132 | 147 |
| TFORF0692-HNF4A | 43 | TFORF3375-TGIF2 | 77 | TFORF2071-MYB | 114 | TFORF0049-MAX | 148 |
| TFORF0693-HNF4A | 43 | TFORF3461-ETV1 | 77 | TFORF2074-MYB | 114 | TFORF0063-GTF3C2 | 148 |
| TFORF0694-HNF4A | 43 | TFORF3529-VDR | 77 | TFORF2456-IRF3 | 114 | TFORF0072-CSDE1 | 148 |
| TFORF0695-HNF4A | 43 | TFORF3544-NR2E1 | 77 | TFORF2526-ERCC2 | 114 | TFORF0122-BARX1 | 148 |
| TFORF1115-RCOR2 | 43 | TFORF0454-TAF4B | 78 | TFORF2540-SOX5 | 114 | TFORF0187-DMRTC2 | 148 |
| TFORF1976-TBX1 | 43 | TFORF0607-CREBL2 | 78 | TFORF2581-MEIS3 | 114 | TFORF0274-TCF3 | 148 |
| TFORF2562-ZNF655 | 43 | TFORF0740-JDP2 | 78 | TFORF2653-ARNTL2 | 114 | TFORF0378-SMARCA1 | 148 |
| TFORF2696-ELF5 | 43 | TFORF0964-ERG | 78 | TFORF2750-NKX6-1 | 114 | TFORF0395-ZNF7 | 148 |
| TFORF2954-HNF4G | 43 | TFORF1118-SKP2 | 78 | TFORF2965-NR2C2 | 114 | TFORF0512-TSHZ1 | 148 |
| TFORF2957-HNF4A | 43 | TFORF1431-ZNF219 | 78 | TFORF3046-ZNF418 | 114 | TFORF0833-GTF2F2 | 148 |
| TFORF3005-SMAD4 | 43 | TFORF1734-RUVBL1 | 78 | TFORF3112-RFXANK | 114 | TFORF0859-ZIC3 | 148 |
| TFORF3542-ZNF720 | 43 | TFORF2192-RFXANK | 78 | TFORF3171-PSMB4 | 114 | TFORF0926-ATOH7 | 148 |
| TFORF0083-KCNIP2 | 44 | TFORF2813-FOXD4L1 | 78 | TFORF3177-NR1D2 | 114 | TFORF0947-EMX2 | 148 |
| TFORF0143-FXN | 44 | TFORF2987-RORA | 78 | TFORF3194-TLX3 | 114 | TFORF0977-ZNF121 | 148 |
| TFORF0217-FOXP1 | 44 | TFORF3176-ZNF544 | 78 | TFORF3200-RUNX3 | 114 | TFORF0995-ZNF304 | 148 |
| TFORF0222-FOXP4 | 44 | TFORF3339-HDAC3 | 78 | TFORF3255-ZNF175 | 114 | TFORF1073-NFATC4 | 148 |
| TFORF0311-CREM | 44 | TFORF3348-PITX2 | 78 | TFORF3404-ZNF511 | 114 | TFORF1089-TULP1 | 148 |
| TFORF0655-NR1I3 | 44 | TFORF3368-FOXR1 | 78 | TFORF0355-ZNF33A | 115 | TFORF1203-SETDB2 | 148 |
| TFORF0665-SCX | 44 | TFORF3445-PTTG1 | 78 | TFORF0575-HOXA11 | 115 | TFORF1306-ZSCAN9 | 148 |
| TFORF1016-ZNF549 | 44 | TFORF3522-POU6F1 | 78 | TFORF2491-ZBTB17 | 115 | TFORF1308-JARID2 | 148 |
| TFORF1084-SPZ1 | 44 | TFORF0159-EWSR1 | 79 | TFORF2902-DACH1 | 115 | TFORF1388-PMS1 | 148 |
| TFORF1498-RBL1 | 44 | TFORF0344-ETF1 | 79 | TFORF2996-TGIF1 | 115 | TFORF1550-TAZ | 148 |
| TFORF1632-ZNF227 | 44 | TFORF0371-NFYC | 79 | TFORF3467-SUPT4H1 | 115 | TFORF1679-RUNX1T1 | 148 |
| TFORF1653-SPI1 | 44 | TFORF0383-SMARCA2 | 79 | TFORF3533-CDIP1 | 115 | TFORF1838-EZH2 | 148 |
| TFORF1703-E2F3 | 44 | TFORF0643-NR1I3 | 79 | TFORF0108-TRERF1 | 116 | TFORF1938-MEIS1 | 148 |
| TFORF1744-ZNF764 | 44 | TFORF0817-LHX3 | 79 | TFORF0900-ZNF382 | 116 | TFORF2106-ZBTB37 | 148 |
| TFORF1918-RUNX3 | 44 | TFORF0871-NR1H3 | 79 | TFORF0973-ZNF124 | 116 | TFORF2123-ZNF273 | 148 |
| TFORF1920-RUNX1 | 44 | TFORF1024-FOXJ3 | 79 | TFORF1036-WT1 | 116 | TFORF2219-YAP1 | 148 |
| TFORF2113-SIRT6 | 44 | TFORF1043-TSC22D3 | 79 | TFORF1513-NKX3-1 | 116 | TFORF2237-GATA4 | 148 |
| TFORF2330-CIZ1 | 44 | TFORF1050-HKR1 | 79 | TFORF2151-FOXG1 | 116 | TFORF2244-TCF15 | 148 |
| TFORF2389-ZNF391 | 44 | TFORF1389-PMS1 | 79 | TFORF2490-ZBTB17 | 116 | TFORF2293-HES7 | 148 |
| TFORF2505-PML | 44 | TFORF1655-SPIB | 79 | TFORF2607-DMTF1 | 116 | TFORF2295-TAF11 | 148 |
| TFORF2563-ZNF655 | 44 | TFORF1700-E2F5 | 79 | TFORF2666-ZBED3 | 116 | TFORF2301-HIC2 | 148 |
| TFORF3271-EBF3 | 44 | TFORF1712-DPRX | 79 | TFORF2729-ZNF668 | 116 | TFORF2344-MITF | 148 |
| TFORF3391-CNBP | 44 | TFORF1747-ZNF761 | 79 | TFORF0431-AIRE | 117 | TFORF2451-MLLT10 | 148 |
| TFORF3502-VENTX | 44 | TFORF1979-TBX6 | 79 | TFORF0458-TRIM24 | 117 | TFORF2518-ZFP37 | 148 |
| TFORF0709-ZNF629 | 45 | TFORF1985-ZNF534 | 79 | TFORF0561-ZNF616 | 117 | TFORF2537-SOX7 | 148 |
| TFORF1242-LBX1 | 45 | TFORF2007-PREB | 79 | TFORF0842-ZSCAN10 | 117 | TFORF2946-MYOD1 | 148 |
| TFORF1681-SIM2 | 45 | TFORF2016-PITX2 | 79 | TFORF0922-NR2F1 | 117 | TFORF2956-HNF4A | 148 |
| TFORF1773-ZNF501 | 45 | TFORF2045-ARID3B | 79 | TFORF1265-ZNF135 | 117 | TFORF2960-PGR | 148 |
| TFORF1837-EZH2 | 45 | TFORF2082-IGHMBP2 | 79 | TFORF1319-KLF13 | 117 | TFORF3067-ZNF8 | 148 |
| TFORF1883-SOX30 | 45 | TFORF2118-TBPL1 | 79 | TFORF1337-ZBTB7A | 117 | TFORF3077-AP2B1 | 148 |
| TFORF1968-YY1 | 45 | TFORF2212-CREB3L3 | 79 | TFORF1538-ZBTB46 | 117 | TFORF3091-ZNF260 | 148 |
| TFORF2112-SIRT6 | 45 | TFORF2217-YAP1 | 79 | TFORF1964-CUX1 | 117 | TFORF3120-ZNF341 | 148 |
| TFORF2565-ZNF655 | 45 | TFORF2393-ZNF394 | 79 | TFORF2289-HES5 | 117 | TFORF3155-HLX | 148 |
| TFORF2592-ZNF354B | 45 | TFORF2470-PDX1 | 79 | TFORF2380-MBD1 | 117 | TFORF3161-ZNF433 | 148 |
| TFORF3034-OTX1 | 45 | TFORF2564-ZNF655 | 79 | TFORF2527-ERCC3 | 117 | TFORF3162-ZNF253 | 148 |
| TFORF3186-GTF2H3 | 45 | TFORF2593-BHLHE40 | 79 | TFORF2553-AR | 117 | TFORF3163-PPP1R13B | 148 |
| TFORF0711-ZNF625 | 46 | TFORF2637-TFDP2 | 79 | TFORF3538-HOXD10 | 117 | TFORF3206-TBR1 | 148 |
| TFORF1049-HKR1 | 46 | TFORF2807-ETV7 | 79 | TFORF0024-FOXQ1 | 118 | TFORF3213-ZNF572 | 148 |
| TFORF1282-SALL4 | 46 | TFORF3076-GTF2A2 | 79 | TFORF0039-SP7 | 118 | TFORF3229-MAX | 148 |
| TFORF1447-NR5A2 | 46 | TFORF3261-OVOL1 | 79 | TFORF0136-ZNF780B | 118 | TFORF3248-ZNF311 | 148 |
| TFORF1448-NR5A2 | 46 | TFORF0141-POU5F1B | 80 | TFORF0166-SIX6 | 118 | TFORF3277-ZNF518A | 148 |
| TFORF2641-PAX3 | 46 | TFORF0229-ZNF595 | 80 | TFORF0173-RELB | 118 | TFORF3296-HIF1A | 148 |
| TFORF2922-NFIA | 46 | TFORF0356-ZNF33A | 80 | TFORF0232-ZNF599 | 118 | TFORF3307-ZNF563 | 148 |
| TFORF3040-ZIM3 | 46 | TFORF0510-TSHZ3 | 80 | TFORF0459-CREB5 | 118 | TFORF3310-ZNF561 | 148 |
| TFORF3131-NR5A2 | 46 | TFORF0731-PROX1 | 80 | TFORF0489-ZFP91 | 118 | TFORF3359-ZNF263 | 148 |
| TFORF3363-NR5A1 | 46 | TFORF0737-ZNF480 | 80 | TFORF0568-ZNF618 | 118 | TFORF3365-FOXA1 | 148 |
| TFORF0184-SHOX2 | 47 | TFORF0827-LHX9 | 80 | TFORF0590-FOXO6 | 118 | TFORF3409-ZNF621 | 148 |
| TFORF0255-TCF4 | 47 | TFORF1666-TP73 | 80 | TFORF0598-SMARCB1 | 118 | TFORF3413-ZFP82 | 148 |
| TFORF0548-PAX8 | 47 | TFORF1701-E2F5 | 80 | TFORF0649-NR1I3 | 118 | TFORF3415-NKRF | 148 |
| TFORF0787-REPIN1 | 47 | TFORF1875-CCNT1 | 80 | TFORF0885-PPARG | 118 | TFORF3432-NR3C1 | 148 |
| TFORF0888-ZNF384 | 47 | TFORF1925-ZBTB21 | 80 | TFORF1174-NRL | 118 | TFORF3451-ZNF366 | 148 |
| TFORF0958-ZNF496 | 47 | TFORF2024-NPAS3 | 80 | TFORF1189-ZNF571 | 118 | TFORF3473-RUNX1 | 148 |
| TFORF1056-NFATC1 | 47 | TFORF2206-POU2F3 | 80 | TFORF1338-ZBTB7C | 118 | TFORF3487-NFATC1 | 148 |
| TFORF1185-MAP3K7 | 47 | TFORF2257-ARNTL | 80 | TFORF1394-ZNF569 | 118 | TFORF3508-ZNF134 | 148 |
| TFORF1706-E2F1 | 47 | TFORF2417-ZNF268 | 80 | TFORF1420-ZNF211 | 118 | TFORF3519-TCF4 | 148 |
| TFORF1791-FOXE3 | 47 | TFORF2506-PML | 80 | TFORF1435-NFKBIE | 118 | TFORF3526-THAP12 | 148 |
| TFORF2312-ETV3 | 47 | TFORF2547-CEBPD | 80 | TFORF1868-MMP3 | 118 | TFORF3536-HOXD4 | 148 |
| TFORF2424-RBPJL | 47 | TFORF2688-TEAD3 | 80 | TFORF1942-MIER1 | 118 | TFORF3549-GFP | 148 |
| TFORF2504-PML | 47 | TFORF2889-BRPF1 | 80 | TFORF2056-NKX2-8 | 118 | TFORF3550-mCherry | 148 |
| TFORF3164-RUVBL1 | 47 | TFORF3243-ZNF267 | 80 | TFORF2097-ZKSCAN1 | 118 | TFORF0050-MAZ | 149 |
| TFORF3196-ZIC1 | 47 | TFORF0162-PES1 | 81 | TFORF2103-ZBTB32 | 118 | TFORF0254-CAMTA1 | 149 |
| TFORF3211-TADA2B | 47 | TFORF0293-MEF2A | 81 | TFORF2457-IRF3 | 118 | TFORF0302-CREM | 149 |
| TFORF3436-TOX4 | 47 | TFORF0366-NFYC | 81 | TFORF2549-SKI | 118 | TFORF0313-CREM | 149 |
| TFORF0491-ZSCAN1 | 48 | TFORF0435-ZSCAN32 | 81 | TFORF3054-SALL4 | 118 | TFORF0416-ZNF559-ZNF177 | 149 |
| TFORF0948-EMX2 | 48 | TFORF0463-HOXD1 | 81 | TFORF3317-ZNF75A | 118 | TFORF0531-PAX6 | 149 |
| TFORF1014-ZNF540 | 48 | TFORF0586-SRY | 81 | TFORF3398-HLF | 118 | TFORF0735-ZNF480 | 149 |
| TFORF2366-MBD4 | 48 | TFORF0699-BATF2 | 81 | TFORF3491-NR1I3 | 118 | TFORF0850-ZIC4 | 149 |
| TFORF3457-IRF9 | 48 | TFORF0710-ZNF624 | 81 | TFORF0186-DMRTC1 | 119 | TFORF0882-HOXB1 | 149 |
| TFORF3534-BORCS8-MEF2B | 48 | TFORF1401-ZNF567 | 81 | TFORF0251-VEZF1 | 119 | TFORF0904-DNAJC1 | 149 |
| TFORF0044-ZNF672 | 49 | TFORF1628-FOXJ3 | 81 | TFORF0320-MSLN | 119 | TFORF1004-POU1F1 | 149 |
| TFORF0316-MEF2D | 49 | TFORF1794-CNBP | 81 | TFORF0864-GTF21 | 119 | TFORF1047-SNAI2 | 149 |
| TFORF0423-PBX3 | 49 | TFORF1998-ZNF248 | 81 | TFORF0956-MYF5 | 119 | TFORF1068-NFATC2 | 149 |
| TFORF1303-ZMIZ2 | 49 | TFORF2147-ZNF528 | 81 | TFORF1114-RCOR3 | 119 | TFORF1162-NFKBIL1 | 149 |
| TFORF2190-ZNF410 | 49 | TFORF2434-ZNF280D | 81 | TFORF1199-ZNF579 | 119 | TFORF1241-LBX2 | 149 |
| TFORF2198-MLXIPL | 49 | TFORF2918-NFIB | 81 | TFORF1234-AFF2 | 119 | TFORF1263-TP53 | 149 |
| TFORF2622-CRTC1 | 49 | TFORF2948-HOXB6 | 81 | TFORF1261-TP53 | 119 | TFORF1384-ZNF788 | 149 |
| TFORF2799-TAF6 | 49 | TFORF2968-NR0B1 | 81 | TFORF1492-FOSL1 | 119 | TFORF1496-HHEX | 149 |
| TFORF2896-MTA2 | 49 | TFORF2995-SKP2 | 81 | TFORF1615-GSX1 | 119 | TFORF1545-ZNF160 | 149 |
| TFORF2908-NFIC | 49 | TFORF3215-ZNF248 | 81 | TFORF1637-HOXC12 | 119 | TFORF1646-ZNF423 | 149 |
| TFORF3031-STRAP | 49 | TFORF3387-ZNF586 | 81 | TFORF2062-NKX2-6 | 119 | TFORF1710-CBFB | 149 |
| TFORF3246-HOXC9 | 49 | TFORF3485-ATF2 | 81 | TFORF2068-FLII | 119 | TFORF1800-USF2 | 149 |
| TFORF3336-ZBTB37 | 49 | TFORF0071-CSDE1 | 82 | TFORF2104-ZBTB34 | 119 | TFORF1811-ZNF438 | 149 |
| TFORF3356-NRF1 | 49 | TFORF0239-MYCL | 82 | TFORF2154-BCL11A | 119 | TFORF1867-POU2F2 | 149 |
| TFORF0001-HIF3A | 50 | TFORF0363-TCEB2 | 82 | TFORF2169-BLZF1 | 119 | TFORF2044-ARID3C | 149 |
| TFORF0265-TCF4 | 50 | TFORF0372-NFYB | 82 | TFORF2290-HES6 | 119 | TFORF2126-ZNF746 | 149 |
| TFORF0334-ZNF331 | 50 | TFORF0402-ZNF3 | 82 | TFORF2728-ZFP1 | 119 | TFORF2220-YAP1 | 149 |
| TFORF0841-ZSCAN10 | 50 | TFORF0425-PBX3 | 82 | TFORF2780-ZNF30 | 119 | TFORF2415-ZNF268 | 149 |
| TFORF1057-NFATC1 | 50 | TFORF0529-PAX6 | 82 | TFORF2982-ZNF420 | 119 | TFORF2600-POU6F2 | 149 |
| TFORF1101-GTF2IRD1 | 50 | TFORF0877-GLMP | 82 | TFORF3011-ZNF10 | 119 | TFORF2619-ZXDB | 149 |
| TFORF1122-HNRNPAB | 50 | TFORF0933-ZNF85 | 82 | TFORF3104-NKX2-5 | 119 | TFORF2850-HMGA2 | 149 |
| TFORF1257-TP53 | 50 | TFORF1026-RARA | 82 | TFORF3242-ZNF136 | 119 | TFORF2897-MTA2 | 149 |
| TFORF1817-UHRF1 | 50 | TFORF1195-ZNF576 | 82 | TFORF3323-ZNF626 | 119 | TFORF2978-NR0B2 | 149 |
| TFORF1898-PATZ1 | 50 | TFORF1792-CNBP | 82 | TFORF3497-ZNF280C | 119 | TFORF3017-ETV4 | 149 |
| TFORF1904-TCF7L2 | 50 | TFORF2017-PITX2 | 82 | TFORF3539-ZNF747 | 119 | TFORF3039-NR1H3 | 149 |
| TFORF2143-ZNF525 | 50 | TFORF2100-SOHLH2 | 82 | TFORF0421-SKIL | 120 | TFORF3184-HSBP1 | 149 |
| TFORF2230-EHF | 50 | TFORF2248-TCF12 | 82 | TFORF0637-HOXC4 | 120 | TFORF3241-ZNF490 | 149 |
| TFORF2335-CTNNB1 | 50 | TFORF2304-CIITA | 82 | TFORF1508-MTF1 | 120 | TFORF3288-HOXA9 | 149 |
| TFORF2351-ZNF17 | 50 | TFORF2313-ETV3 | 82 | TFORF1708-PPP1R13L | 120 | TFORF3408-ZNF286A | 149 |
| TFORF2558-TLX1 | 50 | TFORF2362-ZNF641 | 82 | TFORF1803-USF1 | 120 | TFORF3434-ZNF37A | 149 |
| TFORF2568-TGIF1 | 50 | TFORF2454-IRF3 | 82 | TFORF2159-BCL11B | 120 | TFORF3438-GCM1 | 149 |
| TFORF2650-ZNF586 | 50 | TFORF2550-BID | 82 | TFORF2222-YAP1 | 120 | TFORF3449-DMRT1 | 149 |
| TFORF2724-ZNF74 | 50 | TFORF2849-HMGA2 | 82 | TFORF2396-ZNF396 | 120 | TFORF0309-CREM | 150 |
| TFORF2930-NFIX | 50 | TFORF2969-PAX8 | 82 | TFORF2559-TLX1 | 120 | TFORF0407-CTCFL | 150 |
| TFORF2984-MAP3K7 | 50 | TFORF3014-CEBPG | 82 | TFORF3116-HEYL | 120 | TFORF0410-CTCFL | 150 |
| TFORF3084-ZFP36L1 | 50 | TFORF3240-POU2AF1 | 82 | TFORF0112-SMAD2 | 121 | TFORF0429-PBX1 | 150 |
| TFORF3352-HESX1 | 50 | TFORF3319-ZSCAN5A | 82 | TFORF0790-DBX1 | 121 | TFORF0492-ZSCAN2 | 150 |
| TFORF3455-ZNF513 | 50 | TFORF3532-LIN28B | 82 | TFORF0943-LMO2 | 121 | TFORF0623-DDIT3 | 150 |
| TFORF0213-FOXP1 | 51 | TFORF0079-KCNIP3 | 83 | TFORF1180-ZNF799 | 121 | TFORF0760-OVOL1 | 150 |
| TFORF0420-SKIL | 51 | TFORF0087-LIN28A | 83 | TFORF1300-MAEL | 121 | TFORF0823-LHX6 | 150 |
| TFORF0844-ZSCAN12 | 51 | TFORF0234-MNX1 | 83 | TFORF1464-VAV1 | 121 | TFORF0847-YBX3 | 150 |
| TFORF0982-KLF6 | 51 | TFORF0261-TCF4 | 83 | TFORF1602-ZNF256 | 121 | TFORF1127-THAP3 | 150 |
| TFORF1088-TULP3 | 51 | TFORF0272-TCF7 | 83 | TFORF1903-TCF7L2 | 121 | TFORF1198-ZNF578 | 150 |
| TFORF1627-FOXJ3 | 51 | TFORF0284-MEF2C | 83 | TFORF2066-FLII | 121 | TFORF1318-MKX | 150 |
| TFORF1721-DMRT2 | 51 | TFORF0298-CREM | 83 | TFORF2245-TCF12 | 121 | TFORF1333-TFAP2D | 150 |
| TFORF2233-SMARCD1 | 51 | TFORF0373-NFYA | 83 | TFORF2472-HDAC5 | 121 | TFORF1451-DLX1 | 150 |
| TFORF2408-FOXF1 | 51 | TFORF0417-SULT2A1 | 83 | TFORF2762-ZKSCAN3 | 121 | TFORF1455-TFEC | 150 |
| TFORF2524-ERCC8 | 51 | TFORF0669-ZNF821 | 83 | TFORF2883-BHLHA9 | 121 | TFORF1467-HOXD13 | 150 |
| TFORF2997-GATA2 | 51 | TFORF0798-ATF3 | 83 | TFORF2914-NFIB | 121 | TFORF1497-LYL1 | 150 |
| TFORF3428-TRIM27 | 51 | TFORF0848-YBX3 | 83 | TFORF3018-NEUROG3 | 121 | TFORF1514-NKX3-1 | 150 |
| TFORF1097-HSFX1 | 52 | TFORF0872-HOXB8 | 83 | TFORF3397-ZIK1 | 121 | TFORF1559-YAF2 | 150 |
| TFORF1344-FEZF2 | 52 | TFORF0978-KLF7 | 83 | TFORF3456-ZNF426 | 121 | TFORF1722-DMRT3 | 150 |
| TFORF1386-ZNF362 | 52 | TFORF1145-CDK1 | 83 | TFORF3501-CERS6 | 121 | TFORF1723-ZNF691 | 150 |
| TFORF1526-MXI1 | 52 | TFORF1177-ZNF793 | 83 | TFORF0098-MSGN1 | 122 | TFORF1821-ZNF138 | 150 |
| TFORF1715-LDB2 | 52 | TFORF1364-ELOF1 | 83 | TFORF0163-PES1 | 122 | TFORF1866-POU2F2 | 150 |
| TFORF1781-ZNF506 | 52 | TFORF1446-GTF2H3 | 83 | TFORF0174-RELA | 122 | TFORF2001-MYEF2 | 150 |
| TFORF2195-MLXIPL | 52 | TFORF1486-NANOG | 83 | TFORF0409-CTCFL | 122 | TFORF2028-NPAS1 | 150 |
| TFORF2232-EHF | 52 | TFORF1574-ZNF174 | 83 | TFORF0794-ATF7 | 122 | TFORF2122-ZNF563 | 150 |
| TFORF2843-RHOXF2B | 52 | TFORF1589-ZNF773 | 83 | TFORF0813-CDCA7L | 122 | TFORF2242-GATA1 | 150 |
| TFORF2881-XBP1 | 52 | TFORF1611-GSX2 | 83 | TFORF0970-ZNF124 | 122 | TFORF2385-MBD3 | 150 |
| TFORF2916-NFIB | 52 | TFORF1683-TWIST1 | 83 | TFORF1139-LRRFIP1 | 122 | TFORF2394-ZNF396 | 150 |
| TFORF2991-ID2 | 52 | TFORF1691-PGR | 83 | TFORF1239-ZNF431 | 122 | TFORF2512-PDCD2 | 150 |
| TFORF3134-TFE3 | 52 | TFORF1728-ZNF695 | 83 | TFORF1245-PKNOX2 | 122 | TFORF2517-PDCD2 | 150 |
| TFORF3144-MIER2 | 52 | TFORF1767-ZNF22 | 83 | TFORF1310-TTF1 | 122 | TFORF2551-BID | 150 |
| TFORF3172-STAT6 | 52 | TFORF1840-GTF2A1 | 83 | TFORF1624-ZNF516 | 122 | TFORF2749-NKX6-3 | 150 |
| TFORF3174-ARID5A | 52 | TFORF2183-ZNF415 | 83 | TFORF1894-AHRR | 122 | TFORF2815-FOSB | 150 |
| TFORF3311-CREB3 | 52 | TFORF2211-CREB3L3 | 83 | TFORF1896-NR2C1 | 122 | TFORF2851-HMGA2 | 150 |
| TFORF3314-SMAD9 | 52 | TFORF2223-YAP1 | 83 | TFORF1932-JUN | 122 | TFORF2874-ELF1 | 150 |
| TFORF0605-PUF60 | 53 | TFORF2285-HES3 | 83 | TFORF2022-NPAS4 | 122 | TFORF3301-OLIG3 | 150 |
| TFORF0897-ING1 | 53 | TFORF2419-ZNF268 | 83 | TFORF2089-ZNF442 | 122 | TFORF3304-MITF | 150 |
| TFORF1010-ZNF547 | 53 | TFORF2448-GBX2 | 83 | TFORF2092-ZBTB2 | 122 | TFORF1457-TFEB | 151 |
| TFORF1217-PRRX1 | 53 | TFORF2525-ERCC2 | 83 | TFORF2284-HES2 | 122 | TFORF2341-MITF | 151 |
| TFORF1260-TP53 | 53 | TFORF2579-MEIS3 | 83 | TFORF2318-ZNF732 | 122 | TFORF2342-MITF | 151 |
| TFORF1511-STAT5A | 53 | TFORF2597-RORA | 83 | TFORF2412-ZNF705D | 122 | TFORF2345-MITF | 151 |
| TFORF1561-ZNF688 | 53 | TFORF2632-TFDP3 | 83 | TFORF2478-MESP1 | 122 | TFORF2346-MITF | 151 |
| TFORF1977-TBX1 | 53 | TFORF2812-ETV7 | 83 | TFORF2531-SOX9 | 122 | TFORF3303-MITF | 151 |
| TFORF2005-ZNF664 | 53 | TFORF2814-FOSB | 83 | TFORF2754-ZNF446 | 122 | TFORF3327-LHX6 | 151 |
| TFORF2317-ETV4 | 53 | TFORF2825-IKZF1 | 83 | TFORF3119-DDIT3 | 122 | | |
| TFORF3032-SIRT6 | 53 | TFORF3089-ZSCAN9 | 83 | TFORF3224-HMGB1 | 122 | | |
| TFORF3528-NME2 | 53 | TFORF3150-ZNF254 | 83 | TFORF3249-ZNF181 | 122 | | |
| | | TFORF3173-STAT6 | 83 | TFORF3252-ZNF77 | 122 | | |
| | | TFORF3245-MAFG | 83 | TFORF3399-POU2F2 | 122 | | |
| | | TFORF3278-MEOX1 | 83 | | | | |
| | | TFORF3328-ZSCAN16 | 83 | | | | |

TABLE 19D
Predicted triple TF combinations for reference cell types from the human fetal cell atlas (42). Combinations were ranked based on the cell
type-specific gene signature score. Only the top 100 ranked combinations are shown. Combinations are presented as clusters from (C).

| | | Antigen presenting | | | | | | Bronchiolar and | | CCL19_CCL21 | |
|---|---|---|---|---|---|---|---|---|---|---|---|
| Amacrine cells | | cells | | Astrocytes | | Bipolar cells | | alveolar epithelial cells | | positive cells | |
| Clusters | Score | Clusters | Score | Clusters | Score | Clusters | Score | Clusters | Score | Clusters | Score |
| 23; 89; 118 | 5.96 | 29; 31; 80 | 3.46 | 24; 135; 136 | 7.96 | 11; 42; 81 | 6.19 | 3; 64; 105 | 2.81 | 77; 114; 121 | 9.48 |
| 7; 23; 102 | 5.96 | 22; 31; 53 | 3.46 | 26; 39; 136 | 7.96 | 11; 42; 78 | 6.19 | 18; 105; 130 | 2.81 | 71; 86; 114 | 9.48 |
| 26; 87; 97 | 4.93 | 24; 31; 40 | 3.46 | 24; 79; 136 | 7.96 | 11; 42; 134 | 6.19 | 32; 105; 132 | 2.81 | 71; 84; 114 | 9.48 |
| 23; 76; 102 | 4.93 | 31; 41; 53 | 3.46 | 24; 67; 136 | 7.96 | 11; 42; 119 | 6.19 | 64; 105; 132 | 2.81 | 71; 79; 114 | 9.48 |
| 25; 54; 102 | 4.93 | 31; 41; 86 | 3.46 | 23; 39; 136 | 7.96 | 11; 42; 149 | 6.19 | 18; 41; 105 | 2.81 | 71; 114; 150 | 9.48 |
| 29; 93; 118 | 4.93 | 31; 41; 88 | 3.46 | 39; 136; 150 | 7.96 | 11; 42; 126 | 6.19 | 29; 39; 105 | 2.81 | 86; 114; 120 | 9.48 |
| 21; 30; 102 | 4.93 | 62; 63; 108 | 3.46 | 39; 67; 136 | 7.96 | 11; 42; 74 | 6.19 | 60; 105; 150 | 2.81 | 94; 114; 121 | 9.48 |
| 94; 118; 126 | 4.93 | 25; 31; 126 | 3.46 | 39; 136; 148 | 7.96 | 11; 37; 97 | 6.19 | 52; 53; 105 | 2.81 | 63; 71; 114 | 9.48 |
| 26; 38; 97 | 4.93 | 18; 41; 74 | 3.46 | 39; 136; 147 | 7.96 | 11; 42; 93 | 6.19 | 39; 70; 105 | 2.81 | 71; 82; 114 | 9.48 |
| 99; 102; 116 | 4.93 | 31; 39; 41 | 3.46 | 39; 131; 136 | 7.96 | 11; 28; 42 | 5.02 | 29; 96; 105 | 2.81 | 7; 114; 121 | 9.48 |
| 7; 25; 118 | 4.93 | 24; 31; 41 | 2.58 | 24; 77; 136 | 7.96 | 13; 44; 101 | 5.02 | 41; 105; 148 | 2.81 | 71; 74; 114 | 9.48 |
| 2; 23; 102 | 4.93 | 30; 41; 66 | 2.58 | 24; 39; 136 | 7.96 | 11; 42; 77 | 5.02 | 40; 105; 148 | 2.81 | 71; 75; 114 | 9.48 |
| 23; 97; 118 | 4.93 | 14; 28; 31 | 2.58 | 39; 135; 136 | 7.96 | 13; 42; 101 | 5.02 | 32; 105; 134 | 2.81 | 86; 114; 121 | 8.32 |
| 26; 89; 118 | 4.93 | 5; 31; 86 | 2.58 | 24; 65; 136 | 7.96 | 13; 72; 78 | 5.02 | 41; 105; 138 | 2.81 | 80; 114; 121 | 8.32 |
| 18; 93; 118 | 4.93 | 30; 41; 63 | 2.58 | 4; 39; 136 | 7.96 | 13; 70; 133 | 5.02 | 40; 105; 147 | 2.81 | 114; 121; 142 | 8.32 |
| 53; 63; 118 | 4.93 | 30; 41; 62 | 2.58 | 24; 120; 136 | 7.96 | 11; 13; 44 | 5.02 | 41; 105; 134 | 2.81 | 114; 121; 143 | 8.32 |
| 23; 25; 102 | 4.93 | 62; 91; 114 | 2.58 | 39; 83; 136 | 7.96 | 11; 13; 42 | 5.02 | 41; 105; 133 | 2.81 | 63; 77; 121 | 8.32 |
| 29; 30; 102 | 4.93 | 31; 39; 135 | 2.58 | 24; 109; 136 | 7.96 | 13; 21; 101 | 5.02 | 41; 105; 132 | 2.81 | 7; 114; 120 | 8.32 |
| 23; 30; 102 | 4.93 | 31; 39; 134 | 2.58 | 39; 106; 136 | 7.96 | 11; 37; 96 | 5.02 | 37; 53; 105 | 2.81 | 71; 114; 126 | 8.32 |
| 25; 30; 102 | 4.93 | 24; 31; 80 | 2.58 | 39; 61; 136 | 7.96 | 11; 42; 147 | 5.02 | 60; 105; 134 | 2.81 | 71; 81; 113 | 8.32 |
| 29; 98; 126 | 4.93 | 41; 68; 86 | 2.58 | 39; 84; 136 | 7.96 | 11; 42; 148 | 5.02 | 60; 105; 133 | 2.81 | 113; 121; 141 | 8.32 |
| 53; 90; 118 | 4.93 | 30; 41; 68 | 2.58 | 1; 39; 136 | 7.96 | 11; 33; 97 | 5.02 | 37; 54; 105 | 2.81 | 33; 121; 141 | 8.32 |
| 23; 33; 89 | 4.93 | 74; 85; 133 | 2.58 | 24; 69; 136 | 7.96 | 13; 37; 101 | 5.02 | 35; 41; 105 | 2.81 | 57; 114; 121 | 8.32 |
| 23; 52; 102 | 4.93 | 24; 31; 77 | 2.58 | 39; 78; 136 | 7.96 | 11; 42; 70 | 5.02 | 39; 83; 105 | 2.81 | 114; 121; 149 | 8.32 |
| 33; 60; 102 | 4.93 | 74; 90; 108 | 2.58 | 29; 79; 136 | 6.62 | 13; 72; 81 | 5.02 | 59; 105; 133 | 2.81 | 79; 113; 120 | 8.32 |
| 23; 33; 102 | 4.93 | 31; 53; 77 | 2.58 | 26; 118; 136 | 6.62 | 13; 33; 101 | 5.02 | 17; 67; 105 | 2.81 | 63; 114; 121 | 8.32 |
| 21; 60; 102 | 4.93 | 31; 66; 77 | 2.58 | 72; 77; 136 | 6.62 | 11; 29; 97 | 5.02 | 60; 69; 105 | 2.81 | 32; 114; 121 | 8.32 |
| 23; 88; 97 | 4.93 | 77; 123; 151 | 2.58 | 76; 79; 136 | 6.62 | 13; 35; 101 | 5.02 | 66; 73; 105 | 2.81 | 71; 113; 146 | 8.32 |
| 25; 47; 102 | 4.93 | 8; 40; 114 | 2.58 | 64; 73; 136 | 6.62 | 11; 42; 96 | 5.02 | 53; 64; 105 | 2.81 | 68; 71; 114 | 8.32 |
| 14; 74; 94 | 4.93 | 14; 30; 41 | 2.58 | 74; 120; 136 | 6.62 | 11; 42; 97 | 5.02 | 35; 40; 105 | 2.81 | 29; 71; 114 | 8.32 |
| 53; 86; 118 | 4.93 | 19; 24; 31 | 2.58 | 26; 74; 136 | 6.62 | 11; 39; 42 | 5.02 | 26; 54; 105 | 2.81 | 81; 113; 120 | 8.32 |
| 23; 62; 102 | 4.93 | 14; 29; 31 | 2.58 | 4; 135; 136 | 6.62 | 11; 42; 98 | 5.02 | 5; 39; 105 | 2.81 | 26; 114; 121 | 8.32 |
| 18; 28; 118 | 4.93 | 18; 41; 61 | 2.58 | 6; 28; 136 | 6.62 | 21; 42; 77 | 5.02 | 39; 129; 137 | 2.81 | 72; 114; 121 | 8.32 |
| 24; 98; 116 | 4.93 | 31; 70; 77 | 2.58 | 26; 75; 136 | 6.62 | 13; 72; 83 | 5.02 | 29; 37; 105 | 2.81 | 113; 120; 150 | 8.32 |
| 8; 16; 23 | 4.93 | 62; 91; 134 | 2.58 | 64; 67; 136 | 6.62 | 11; 78; 81 | 5.02 | 62; 105; 130 | 2.81 | 38; 121; 149 | 8.32 |
| 18; 23; 102 | 4.93 | 74; 84; 108 | 2.58 | 69; 87; 136 | 6.62 | 11; 42; 90 | 5.02 | 29; 105; 132 | 2.81 | 38; 121; 150 | 8.32 |
| 24; 65; 98 | 4.93 | 41; 68; 135 | 2.58 | 6; 29; 136 | 6.62 | 11; 12; 42 | 5.02 | 96; 105; 132 | 2.81 | 29; 114; 121 | 8.32 |
| 24; 88; 98 | 4.93 | 5; 31; 58 | 2.58 | 93; 107; 136 | 6.62 | 11; 81; 93 | 5.02 | 29; 76; 105 | 2.81 | 29; 113; 120 | 8.32 |
| 23; 54; 102 | 4.93 | 31; 53; 140 | 2.58 | 73; 74; 136 | 6.62 | 11; 42; 107 | 5.02 | 15; 105; 132 | 2.81 | 29; 114; 120 | 8.32 |
| 23; 89; 119 | 4.93 | 31; 40; 93 | 2.58 | 52; 135; 136 | 6.62 | 13; 53; 101 | 5.02 | 8; 105; 132 | 2.81 | 33; 71; 114 | 8.32 |
| 99; 101; 116 | 4.93 | 17; 41; 66 | 2.58 | 13; 66; 136 | 6.62 | 12; 13; 36 | 5.02 | 29; 67; 105 | 2.81 | 114; 121; 134 | 8.32 |
| 7; 26; 97 | 4.93 | 20; 41; 114 | 2.58 | 93; 106; 136 | 6.62 | 11; 15; 42 | 5.02 | 54; 66; 105 | 2.81 | 61; 114; 121 | 8.32 |
| 23; 84; 119 | 4.93 | 31; 40; 87 | 2.58 | 61; 98; 136 | 6.62 | 11; 42; 84 | 5.02 | 39; 69; 105 | 2.81 | 1; 114; 121 | 8.32 |
| 33; 102; 135 | 4.93 | 62; 90; 108 | 2.58 | 76; 77; 136 | 6.62 | 21; 39; 42 | 5.02 | 60; 68; 105 | 2.81 | 39; 114; 121 | 8.32 |
| 23; 42; 118 | 4.93 | 24; 53; 114 | 2.58 | 38; 120; 136 | 6.62 | 11; 64; 93 | 5.02 | 3; 67; 105 | 2.81 | 75; 113; 120 | 8.32 |
| 29; 82; 98 | 4.93 | 24; 53; 112 | 2.58 | 68; 70; 136 | 6.62 | 11; 42; 79 | 5.02 | 26; 53; 105 | 2.81 | 33; 114; 120 | 8.32 |
| 85; 86; 118 | 4.93 | 31; 40; 81 | 2.58 | 1; 75; 136 | 6.62 | 11; 75; 97 | 5.02 | 60; 105; 126 | 2.81 | 53; 113; 120 | 8.32 |
| 7; 85; 118 | 4.93 | 31; 40; 80 | 2.58 | 73; 120; 136 | 6.62 | 11; 42; 85 | 5.02 | 40; 105; 138 | 2.81 | 114; 121; 126 | 8.32 |
| 23; 78; 118 | 4.93 | 31; 40; 77 | 2.58 | 29; 38; 136 | 6.62 | 11; 37; 78 | 5.02 | 4; 53; 105 | 2.81 | 8; 114; 121 | 8.32 |
| 7; 84; 118 | 3.96 | 31; 40; 75 | 2.58 | 24; 113; 136 | 6.62 | 11; 43; 97 | 5.02 | 3; 41; 105 | 2.81 | 65; 100; 113 | 8.32 |
| 28; 75; 118 | 3.96 | 26; 31; 41 | 2.58 | 13; 70; 136 | 6.62 | 11; 38; 134 | 5.02 | 21; 59; 105 | 2.81 | 81; 114; 121 | 8.32 |
| 18; 24; 118 | 3.96 | 31; 40; 94 | 2.58 | 8; 127; 136 | 6.62 | 11; 64; 97 | 5.02 | 8; 29; 105 | 2.81 | 34; 114; 121 | 8.32 |
| 51; 99; 102 | 3.96 | 29; 30; 41 | 2.58 | 92; 136; 141 | 6.62 | 13; 63; 81 | 5.02 | 39; 105; 147 | 2.81 | 63; 121; 129 | 8.32 |
| 23; 86; 102 | 3.96 | 1; 31; 66 | 2.58 | 64; 120; 136 | 6.62 | 11; 42; 86 | 5.02 | 39; 105; 148 | 2.81 | 53; 114; 121 | 7.22 |
| 24; 75; 102 | 3.96 | 31; 69; 73 | 2.58 | 92; 136; 146 | 6.62 | 11; 42; 120 | 5.02 | 39; 105; 149 | 2.81 | 53; 114; 120 | 7.22 |
| 24; 102; 118 | 3.96 | 20; 41; 84 | 2.58 | 92; 136; 150 | 6.62 | 11; 42; 87 | 5.02 | 76; 105; 132 | 2.81 | 71; 74; 113 | 7.22 |
| 29; 83; 118 | 3.96 | 22; 31; 34 | 2.58 | 65; 72; 136 | 6.62 | 11; 38; 78 | 5.02 | 2; 105; 134 | 2.81 | 113; 120; 146 | 7.22 |
| 56; 102; 135 | 3.96 | 31; 69; 77 | 2.58 | 65; 107; 136 | 6.62 | 13; 63; 133 | 5.02 | 39; 105; 138 | 2.81 | 70; 71; 113 | 7.22 |
| 7; 26; 118 | 3.96 | 34; 57; 74 | 2.58 | 26; 73; 136 | 6.62 | 1; 77; 93 | 3.94 | 54; 98; 105 | 2.81 | 81; 113; 121 | 7.22 |
| 26; 83; 97 | 3.96 | 41; 66; 123 | 2.58 | 93; 96; 136 | 6.62 | 11; 76; 78 | 3.94 | 54; 67; 105 | 2.81 | 63; 121; 138 | 7.22 |
| 23; 87; 97 | 3.96 | 17; 41; 135 | 2.58 | 92; 131; 136 | 6.62 | 12; 36; 81 | 3.94 | 2; 105; 133 | 2.81 | 113; 120; 147 | 7.22 |
| 7; 25; 119 | 3.96 | 31; 69; 88 | 2.58 | 1; 79; 136 | 6.62 | 11; 86; 97 | 3.94 | 2; 105; 132 | 2.81 | 86; 121; 142 | 7.22 |
| 38; 47; 102 | 3.96 | 28; 29; 31 | 2.58 | 131; 135; 136 | 6.62 | 29; 37; 107 | 3.94 | 54; 76; 105 | 2.81 | 100; 114; 123 | 7.22 |
| 18; 24; 97 | 3.96 | 7; 31; 77 | 2.58 | 26; 44; 136 | 6.62 | 7; 13; 81 | 3.94 | 53; 96; 105 | 2.81 | 86; 121; 141 | 7.22 |
| 54; 102; 133 | 3.96 | 31; 40; 100 | 2.58 | 92; 135; 136 | 6.62 | 11; 79; 81 | 3.94 | 10; 41; 105 | 2.81 | 7; 121; 141 | 7.22 |
| 8; 26; 97 | 3.96 | 62; 73; 108 | 2.58 | 67; 74; 136 | 6.62 | 11; 13; 62 | 3.94 | 39; 61; 105 | 2.81 | 7; 121; 143 | 7.22 |
| 29; 84; 98 | 3.96 | 28; 74; 108 | 2.58 | 54; 96; 136 | 6.62 | 13; 50; 95 | 3.94 | 53; 54; 105 | 2.81 | 113; 120; 126 | 7.22 |
| 54; 102; 132 | 3.96 | 1; 47; 114 | 2.58 | 8; 126; 136 | 6.62 | 11; 13; 81 | 3.94 | 17; 59; 105 | 2.81 | 5; 114; 121 | 7.22 |
| 7; 25; 97 | 3.96 | 4; 31; 66 | 2.58 | 67; 109; 136 | 6.62 | 11; 13; 66 | 3.94 | 35; 105; 130 | 2.81 | 114; 120; 121 | 7.22 |
| 30; 51; 102 | 3.96 | 30; 41; 74 | 2.58 | 13; 68; 136 | 6.62 | 12; 25; 44 | 3.94 | 27; 60; 105 | 2.81 | 7; 100; 113 | 7.22 |
| 26; 84; 97 | 3.96 | 31; 66; 86 | 2.58 | 109; 135; 136 | 6.62 | 12; 36; 39 | 3.94 | 3; 54; 105 | 2.81 | 25; 113; 120 | 7.22 |
| 85; 88; 118 | 3.96 | 18; 41; 135 | 2.58 | 74; 118; 136 | 6.62 | 13; 72; 101 | 3.94 | 67; 105; 132 | 2.81 | 44; 114; 121 | 7.22 |
| 47; 88; 102 | 3.96 | 5; 31; 40 | 2.58 | 8; 120; 136 | 6.62 | 13; 42; 87 | 3.94 | 29; 54; 105 | 2.81 | 21; 114; 121 | 7.22 |
| 29; 85; 98 | 3.96 | 5; 31; 42 | 2.58 | 29; 67; 136 | 6.62 | 14; 95; 132 | 3.94 | 39; 62; 105 | 2.81 | 63; 114; 129 | 7.22 |
| 26; 86; 97 | 3.96 | 74; 82; 108 | 2.58 | 22; 24; 136 | 6.62 | 15; 95; 106 | 3.94 | 54; 96; 105 | 2.81 | 33; 114; 121 | 7.22 |
| 78; 88; 118 | 3.96 | 25; 28; 31 | 2.58 | 66; 96; 136 | 6.62 | 11; 19; 95 | 3.94 | 40; 127; 139 | 2.81 | 22; 114; 121 | 7.22 |
| 24; 64; 118 | 3.96 | 31; 39; 146 | 2.58 | 61; 96; 136 | 6.62 | 11; 13; 63 | 3.94 | 21; 41; 105 | 2.81 | 38; 84; 121 | 7.22 |
| 96; 116; 118 | 3.96 | 30; 41; 73 | 2.58 | 38; 135; 136 | 6.62 | 13; 29; 101 | 3.94 | 17; 54; 105 | 2.81 | 71; 86; 113 | 7.22 |
| 24; 30; 102 | 3.96 | 24; 30; 41 | 2.58 | 6; 39; 136 | 6.62 | 13; 72; 93 | 3.94 | 54; 105; 130 | 2.81 | 114; 116; 121 | 7.22 |
| 25; 33; 118 | 3.96 | 31; 87; 135 | 2.58 | 72; 135; 136 | 6.62 | 11; 26; 97 | 3.94 | 8; 53; 105 | 2.81 | 96; 121; 145 | 7.22 |
| 27; 59; 102 | 3.96 | 5; 31; 53 | 2.58 | 39; 110; 136 | 6.62 | 11; 13; 78 | 3.94 | 39; 105; 120 | 2.81 | 100; 114; 150 | 7.22 |
| 29; 88; 98 | 3.96 | 30; 41; 82 | 2.58 | 14; 45; 136 | 6.62 | 50; 84; 95 | 3.94 | 54; 105; 132 | 2.81 | 81; 114; 120 | 7.22 |
| 23; 85; 102 | 3.96 | 30; 41; 85 | 2.58 | 13; 69; 136 | 6.62 | 16; 29; 66 | 3.94 | 39; 53; 105 | 2.81 | 100; 114; 148 | 7.22 |
| 29; 88; 118 | 3.96 | 25; 29; 31 | 2.58 | 13; 65; 136 | 6.62 | 13; 72; 84 | 3.94 | 54; 105; 135 | 2.81 | 100; 114; 145 | 7.22 |
| 23; 63; 102 | 3.96 | 20; 41; 150 | 2.58 | 60; 97; 136 | 6.62 | 13; 97; 132 | 3.94 | 39; 105; 133 | 2.81 | 8; 121; 145 | 7.22 |
| 25; 72; 118 | 3.96 | 31; 39; 150 | 2.58 | 82; 106; 136 | 6.62 | 11; 67; 89 | 3.94 | 3; 60; 105 | 2.81 | 12; 114; 121 | 7.22 |
| 7; 23; 101 | 3.96 | 30; 41; 79 | 2.58 | 72; 83; 136 | 6.62 | 11; 86; 96 | 3.94 | 59; 86; 105 | 2.81 | 71; 95; 113 | 7.22 |
| 7; 23; 89 | 3.96 | 14; 25; 31 | 2.58 | 44; 120; 136 | 6.62 | 13; 25; 101 | 3.94 | 3; 66; 105 | 2.81 | 70; 71; 114 | 7.22 |
| 47; 82; 102 | 3.96 | 30; 41; 77 | 2.58 | 87; 136; 147 | 6.62 | 11; 13; 28 | 3.94 | 59; 105; 134 | 2.81 | 71; 95; 114 | 7.22 |
| 25; 76; 97 | 3.96 | 31; 40; 44 | 2.58 | 14; 65; 136 | 6.62 | 11; 67; 97 | 3.94 | 8; 54; 105 | 2.81 | 38; 113; 121 | 7.22 |
| 29; 89; 118 | 3.96 | 31; 40; 41 | 2.58 | 101; 135; 136 | 6.62 | 13; 42; 110 | 3.94 | 41; 86; 105 | 2.81 | 63; 114; 120 | 7.22 |
| 7; 57; 102 | 3.96 | 31; 39; 151 | 2.58 | 73; 79; 136 | 6.62 | 12; 35; 64 | 3.94 | 29; 64; 105 | 2.81 | 26; 121; 141 | 7.22 |
| 23; 85; 97 | 3.96 | 31; 60; 87 | 2.58 | 14; 66; 136 | 6.62 | 13; 45; 72 | 3.94 | 41; 44; 105 | 2.81 | 28; 113; 120 | 7.22 |
| 30; 55; 102 | 3.96 | 8; 31; 57 | 2.58 | 59; 130; 131 | 6.62 | 13; 81; 83 | 3.94 | 60; 85; 105 | 2.81 | 90; 114; 121 | 7.22 |
| 23; 78; 89 | 3.96 | 29; 31; 41 | 2.58 | 39; 124; 136 | 6.62 | 35; 53; 101 | 3.94 | 105; 130; 147 | 2.81 | 86; 113; 120 | 7.22 |
| 7; 56; 102 | 3.96 | 29; 31; 45 | 2.58 | 73; 78; 136 | 6.62 | 11; 53; 101 | 3.94 | 3; 39; 105 | 2.81 | 25; 71; 113 | 7.22 |
| 68; 88; 118 | 3.96 | 24; 41; 108 | 2.58 | 93; 120; 136 | 6.62 | 13; 81; 93 | 3.94 | 105; 130; 148 | 2.81 | 37; 113; 121 | 7.22 |
| 26; 62; 97 | 3.96 | 20; 84; 150 | 2.58 | 1; 65; 136 | 6.62 | 11; 53; 97 | 3.94 | 4; 54; 105 | 2.81 | 17; 114; 121 | 7.22 |
| 25; 74; 118 | 3.96 | 8; 31; 48 | 2.58 | 66; 93; 136 | 6.62 | 15; 53; 95 | 3.94 | 41; 62; 105 | 2.81 | 81; 121; 132 | 7.22 |
| 21; 56; 102 | 3.96 | 18; 31; 57 | 2.58 | 7; 67; 136 | 6.62 | 11; 53; 96 | 3.94 | 53; 105; 132 | 2.81 | 38; 85; 121 | 7.22 |

| | | | | | | | | | | Corneal and | |
|---|---|---|---|---|---|---|---|---|---|---|---|
| CLC_IL5RA | | CSH1_CSH2 | | | | | | Ciliated | | conjunctival | |
| positive cells | | positive cells | | Cardiomyocytes | | Chromaffin cells | | epithelial cells | | epithelial cells | |
| Clusters | Score | Clusters | Score | Clusters | Score | Clusters | Score | Clusters | Score | Clusters | Score |
| 63; 74; 138 | 11.31 | 10; 25; 109 | 9.92 | 1; 62; 113 | 12.22 | 19; 75; 101 | 5.19 | 72; 103; 143 | 25.41 | 6; 14; 109 | 7.41 |
| 36; 113; 131 | 11.31 | 74; 94; 147 | 8.53 | 53; 113; 126 | 12.22 | 7; 101; 129 | 5.19 | 57; 62; 103 | 25.41 | 6; 84; 109 | 7.41 |
| 63; 113; 131 | 11.31 | 63; 79; 109 | 8.53 | 62; 113; 126 | 12.22 | 26; 101; 129 | 5.19 | 39; 103; 143 | 25.41 | 6; 7; 109 | 7.41 |
| 53; 71; 138 | 11.31 | 62; 71; 109 | 8.53 | 63; 114; 120 | 12.22 | 29; 37; 101 | 5.19 | 35; 62; 103 | 25.41 | 6; 26; 109 | 7.41 |
| 6; 71; 138 | 11.31 | 10; 63; 109 | 8.53 | 63; 113; 131 | 12.22 | 29; 101; 129 | 5.19 | 10; 103; 129 | 25.41 | 10; 31; 114 | 7.41 |
| 29; 71; 138 | 11.31 | 8; 63; 109 | 8.53 | 1; 113; 131 | 12.22 | 29; 81; 101 | 5.19 | 45; 103; 143 | 25.41 | 31; 70; 77 | 6.03 |
| 1; 113; 131 | 11.31 | 25; 82; 109 | 8.53 | 62; 113; 150 | 12.22 | 19; 101; 129 | 5.19 | 37; 93; 103 | 25.41 | 73; 108; 109 | 6.03 |
| 7; 71; 138 | 11.31 | 8; 38; 109 | 8.53 | 63; 113; 126 | 12.22 | 63; 101; 129 | 5.19 | 37; 62; 103 | 25.41 | 6; 82; 109 | 6.03 |
| 71; 86; 138 | 11.31 | 38; 74; 94 | 8.53 | 62; 113; 147 | 12.22 | 14; 44; 97 | 3.93 | 103; 143; 150 | 25.41 | 73; 109; 126 | 6.03 |
| 36; 113; 138 | 11.31 | 63; 68; 109 | 8.53 | 37; 62; 113 | 12.22 | 29; 72; 101 | 3.93 | 103; 143; 149 | 25.41 | 6; 70; 109 | 6.03 |
| 70; 71; 138 | 11.31 | 74; 84; 94 | 8.53 | 29; 71; 138 | 10.75 | 61; 79; 101 | 3.93 | 103; 143; 148 | 25.41 | 69; 73; 109 | 6.03 |
| 71; 72; 138 | 11.31 | 38; 64; 109 | 7.22 | 62; 113; 129 | 10.75 | 29; 70; 101 | 3.93 | 103; 143; 147 | 25.41 | 31; 63; 114 | 6.03 |
| 29; 113; 131 | 11.31 | 64; 71; 109 | 7.22 | 1; 83; 113 | 10.75 | 26; 35; 101 | 3.93 | 96; 103; 143 | 25.41 | 73; 109; 150 | 6.03 |
| 10; 113; 134 | 11.31 | 64; 94; 109 | 7.22 | 62; 113; 131 | 10.75 | 25; 101; 129 | 3.93 | 82; 103; 143 | 25.41 | 73; 109; 149 | 6.03 |
| 33; 71; 138 | 9.95 | 8; 61; 109 | 7.22 | 62; 75; 113 | 10.75 | 92; 101; 129 | 3.93 | 83; 103; 143 | 25.41 | 7; 109; 148 | 6.03 |
| 65; 113; 131 | 9.95 | 10; 84; 109 | 7.22 | 62; 113; 138 | 10.75 | 14; 44; 119 | 3.93 | 37; 82; 103 | 25.41 | 25; 29; 109 | 6.03 |
| 83; 113; 131 | 9.95 | 37; 94; 109 | 7.22 | 62; 108; 113 | 10.75 | 7; 19; 118 | 3.93 | 75; 103; 129 | 25.41 | 66; 73; 109 | 6.03 |
| 8; 71; 138 | 9.95 | 70; 94; 109 | 7.22 | 53; 101; 113 | 10.75 | 7; 101; 126 | 3.93 | 8; 64; 103 | 25.41 | 73; 79; 109 | 6.03 |
| 61; 71; 138 | 9.95 | 10; 33; 109 | 7.22 | 63; 64; 113 | 10.75 | 86; 101; 129 | 3.93 | 68; 103; 143 | 25.41 | 7; 83; 109 | 6.03 |
| 28; 113; 131 | 9.95 | 8; 64; 109 | 7.22 | 63; 65; 113 | 10.75 | 44; 101; 147 | 3.93 | 89; 103; 143 | 25.41 | 25; 75; 109 | 6.03 |
| 63; 88; 138 | 9.95 | 61; 63; 109 | 7.22 | 63; 66; 113 | 10.75 | 70; 101; 129 | 3.93 | 37; 103; 147 | 25.41 | 73; 75; 109 | 6.03 |
| 45; 113; 131 | 9.95 | 54; 98; 109 | 7.22 | 67; 113; 131 | 10.75 | 7; 101; 108 | 3.93 | 89; 103; 135 | 25.41 | 73; 83; 109 | 6.03 |
| 71; 114; 131 | 9.95 | 10; 57; 109 | 7.22 | 62; 96; 113 | 10.75 | 44; 101; 133 | 3.93 | 37; 103; 149 | 25.41 | 7; 82; 109 | 6.03 |
| 36; 113; 126 | 9.95 | 7; 10; 109 | 7.22 | 36; 101; 113 | 10.75 | 44; 101; 129 | 3.93 | 103; 126; 143 | 25.41 | 10; 31; 141 | 6.03 |
| 71; 84; 138 | 9.95 | 25; 109; 120 | 7.22 | 62; 101; 113 | 10.75 | 44; 101; 107 | 3.93 | 78; 103; 143 | 25.41 | 6; 37; 109 | 6.03 |
| 63; 113; 126 | 9.95 | 71; 109; 142 | 7.22 | 37; 63; 113 | 10.75 | 101; 108; 129 | 3.93 | 44; 103; 143 | 25.41 | 73; 82; 109 | 6.03 |
| 10; 113; 131 | 9.95 | 8; 82; 109 | 7.22 | 71; 113; 134 | 10.75 | 7; 101; 150 | 3.93 | 37; 68; 103 | 25.41 | 6; 41; 109 | 6.03 |
| 64; 72; 113 | 9.95 | 91; 109; 132 | 7.22 | 114; 120; 131 | 10.75 | 29; 68; 101 | 3.93 | 73; 103; 143 | 25.41 | 4; 31; 114 | 6.03 |
| 67; 113; 131 | 9.95 | 63; 109; 132 | 7.22 | 62; 94; 113 | 10.75 | 53; 94; 101 | 3.93 | 57; 103; 129 | 25.41 | 78; 109; 114 | 6.03 |
| 96; 113; 131 | 9.95 | 63; 109; 134 | 7.22 | 62; 93; 113 | 10.75 | 19; 84; 101 | 3.93 | 62; 72; 103 | 25.41 | 7; 84; 109 | 6.03 |
| 71; 73; 138 | 9.95 | 31; 81; 120 | 7.22 | 29; 65; 113 | 10.75 | 34; 92; 101 | 3.93 | 6; 103; 143 | 25.41 | 26; 62; 109 | 6.03 |
| 62; 113; 138 | 9.95 | 8; 25; 109 | 7.22 | 62; 84; 113 | 10.75 | 63; 82; 101 | 3.93 | 84; 103; 143 | 25.41 | 64; 73; 109 | 6.03 |
| 62; 113; 150 | 9.95 | 68; 94; 109 | 7.22 | 62; 83; 113 | 10.75 | 73; 101; 129 | 3.93 | 37; 103; 120 | 25.41 | 6; 68; 109 | 6.03 |
| 113; 120; 131 | 9.95 | 14; 63; 109 | 7.22 | 68; 75; 113 | 10.75 | 70; 102; 129 | 3.93 | 70; 103; 143 | 25.41 | 31; 70; 114 | 6.03 |
| 113; 131; 150 | 9.95 | 71; 79; 109 | 7.22 | 62; 81; 113 | 10.75 | 34; 101; 129 | 3.93 | 76; 103; 129 | 25.41 | 26; 64; 109 | 6.03 |
| 113; 131; 151 | 9.95 | 57; 71; 109 | 7.22 | 53; 113; 150 | 10.75 | 42; 101; 129 | 3.93 | 54; 103; 129 | 25.41 | 25; 73; 109 | 6.03 |
| 64; 113; 131 | 9.95 | 44; 94; 109 | 7.22 | 62; 79; 113 | 10.75 | 28; 101; 129 | 3.93 | 37; 103; 119 | 25.41 | 7; 70; 109 | 6.03 |
| 44; 113; 131 | 9.95 | 31; 71; 94 | 7.22 | 71; 113; 126 | 10.75 | 77; 101; 129 | 3.93 | 37; 70; 103 | 25.41 | 4; 109; 114 | 6.03 |
| 72; 113; 134 | 9.95 | 81; 109; 132 | 7.22 | 71; 114; 131 | 10.75 | 85; 101; 129 | 3.93 | 93; 103; 143 | 25.41 | 6; 73; 109 | 6.03 |
| 79; 113; 131 | 9.95 | 62; 98; 109 | 7.22 | 63; 73; 113 | 10.75 | 36; 81; 101 | 3.93 | 49; 68; 103 | 25.41 | 9; 109; 114 | 6.03 |
| 66; 113; 131 | 9.95 | 57; 90; 109 | 7.22 | 63; 68; 113 | 10.75 | 36; 81; 102 | 3.93 | 5; 57; 103 | 25.41 | 29; 73; 109 | 6.03 |
| 62; 113; 131 | 9.95 | 62; 94; 109 | 7.22 | 63; 88; 138 | 10.75 | 63; 75; 101 | 3.93 | 10; 62; 103 | 25.41 | 31; 68; 114 | 6.03 |
| 71; 75; 138 | 9.95 | 8; 74; 109 | 7.22 | 63; 113; 133 | 10.75 | 18; 92; 101 | 3.93 | 103; 120; 143 | 25.41 | 7; 64; 109 | 6.03 |
| 78; 113; 131 | 9.95 | 25; 81; 94 | 7.22 | 66; 72; 113 | 10.75 | 10; 101; 129 | 3.93 | 79; 103; 143 | 25.41 | 61; 109; 114 | 6.03 |
| 22; 113; 134 | 9.95 | 70; 89; 109 | 7.22 | 63; 113; 132 | 10.75 | 63; 72; 101 | 3.93 | 37; 103; 134 | 25.41 | 29; 66; 109 | 6.03 |
| 18; 113; 131 | 9.95 | 8; 65; 109 | 7.22 | 66; 74; 113 | 10.75 | 70; 81; 102 | 3.93 | 54; 97; 103 | 25.41 | 6; 10; 109 | 6.03 |
| 71; 120; 138 | 9.95 | 20; 25; 148 | 7.22 | 53; 65; 113 | 10.75 | 70; 81; 101 | 3.93 | 96; 103; 129 | 25.41 | 29; 62; 109 | 6.03 |
| 114; 120; 131 | 9.95 | 10; 98; 109 | 7.22 | 1; 65; 113 | 10.75 | 29; 35; 101 | 3.93 | 69; 103; 143 | 25.41 | 68; 73; 109 | 6.03 |
| 25; 113; 131 | 9.95 | 71; 81; 109 | 7.22 | 53; 68; 113 | 10.75 | 53; 101; 129 | 3.93 | 63; 103; 143 | 25.41 | 4; 109; 119 | 6.03 |
| 63; 71; 138 | 9.95 | 25; 83; 109 | 7.22 | 44; 113; 131 | 10.75 | 26; 37; 101 | 3.93 | 66; 103; 129 | 25.41 | 10; 73; 109 | 6.03 |
| 10; 36; 113 | 9.95 | 63; 94; 109 | 7.22 | 79; 113; 131 | 10.75 | 29; 36; 101 | 3.93 | 64; 97; 103 | 25.41 | 4; 109; 148 | 6.03 |
| 47; 113; 131 | 9.95 | 8; 84; 109 | 7.22 | 63; 101; 113 | 10.75 | 29; 38; 101 | 3.93 | 37; 78; 103 | 25.41 | 2; 64; 109 | 6.03 |
| 108; 113; 131 | 9.95 | 68; 89; 109 | 7.22 | 63; 100; 113 | 10.75 | 92; 99; 107 | 3.93 | 88; 103; 143 | 25.41 | 29; 55; 109 | 6.03 |
| 71; 74; 138 | 9.95 | 68; 71; 109 | 7.22 | 53; 71; 138 | 10.75 | 29; 73; 101 | 3.93 | 57; 64; 103 | 25.41 | 4; 26; 109 | 6.03 |
| 51; 113; 134 | 9.95 | 10; 82; 109 | 7.22 | 63; 93; 113 | 10.75 | 11; 44; 68 | 3.93 | 37; 89; 103 | 25.41 | 77; 109; 114 | 6.03 |
| 7; 63; 138 | 9.95 | 38; 109; 132 | 7.22 | 1; 34; 113 | 10.75 | 74; 81; 101 | 3.93 | 66; 86; 103 | 23.3 | 25; 66; 109 | 6.03 |
| 62; 113; 126 | 9.95 | 57; 94; 109 | 7.22 | 53; 81; 113 | 10.75 | 29; 93; 101 | 3.93 | 69; 103; 133 | 23.38 | 69; 109; 114 | 6.03 |
| 44; 71; 138 | 9.95 | 8; 71; 109 | 7.22 | 63; 70; 113 | 10.75 | 76; 101; 129 | 3.93 | 69; 86; 103 | 23.38 | 7; 68; 109 | 6.03 |
| 22; 113; 131 | 9.95 | 25; 74; 109 | 7.22 | 63; 87; 113 | 10.75 | 36; 101; 129 | 3.93 | 7; 66; 103 | 23.38 | 64; 99; 109 | 6.03 |
| 72; 113; 131 | 9.95 | 63; 82; 109 | 7.22 | 66; 113; 131 | 10.75 | 44; 88; 101 | 3.93 | 68; 72; 103 | 23.38 | 4; 14; 109 | 6.03 |
| 53; 113; 126 | 9.95 | 25; 109; 134 | 7.22 | 53; 83; 113 | 10.75 | 7; 75; 101 | 3.93 | 57; 103; 114 | 23.38 | 31; 82; 114 | 6.03 |
| 101; 113; 131 | 9.95 | 25; 84; 109 | 7.22 | 63; 85; 113 | 10.75 | 44; 87; 101 | 3.93 | 42; 61; 103 | 23.38 | 35; 73; 109 | 6.03 |
| 55; 113; 134 | 9.95 | 63; 83; 109 | 7.22 | 63; 84; 113 | 10.75 | 7; 74; 101 | 3.93 | 64; 77; 103 | 23.38 | 64; 109; 114 | 6.03 |
| 71; 113; 134 | 9.95 | 8; 75; 109 | 7.22 | 63; 83; 113 | 10.75 | 29; 101; 108 | 3.93 | 63; 67; 103 | 23.38 | 60; 109; 114 | 6.03 |
| 71; 87; 113 | 9.95 | 10; 38; 109 | 7.22 | 63; 81; 113 | 10.75 | 29; 101; 126 | 3.93 | 54; 96; 103 | 23.38 | 29; 75; 109 | 6.03 |
| 74; 113; 131 | 9.95 | 10; 83; 109 | 7.22 | 1; 72; 113 | 10.75 | 29; 101; 146 | 3.93 | 66; 103; 125 | 23.38 | 109; 114; 140 | 6.03 |
| 71; 133; 148 | 9.95 | 8; 37; 109 | 7.22 | 63; 79; 113 | 10.75 | 29; 101; 147 | 3.93 | 57; 103; 118 | 23.38 | 73; 84; 109 | 6.03 |
| 71; 83; 138 | 9.95 | 109; 120; 132 | 7.22 | 63; 75; 113 | 10.75 | 29; 101; 150 | 3.93 | 35; 82; 103 | 23.38 | 6; 109; 148 | 6.03 |
| 63; 113; 138 | 9.95 | 10; 86; 109 | 7.22 | 63; 74; 138 | 10.75 | 7; 72; 101 | 3.93 | 29; 38; 103 | 23.38 | 14; 66; 109 | 6.03 |
| 71; 79; 138 | 9.95 | 25; 38; 148 | 7.22 | 63; 74; 113 | 10.75 | 75; 81; 101 | 3.93 | 38; 103; 129 | 23.38 | 10; 109; 114 | 6.03 |
| 23; 113; 131 | 9.95 | 25; 38; 149 | 7.22 | 62; 73; 113 | 10.75 | 7; 70; 101 | 3.93 | 38; 45; 103 | 23.38 | 6; 33; 109 | 6.03 |
| 53; 113; 131 | 9.95 | 25; 91; 109 | 7.22 | 63; 72; 113 | 10.75 | 94; 101; 129 | 3.93 | 39; 54; 103 | 23.38 | 70; 73; 109 | 6.03 |
| 49; 113; 131 | 9.95 | 63; 89; 109 | 7.22 | 62; 74; 113 | 10.75 | 60; 81; 101 | 3.93 | 73; 103; 129 | 23.38 | 4; 70; 109 | 6.03 |
| 71; 88; 138 | 9.95 | 65; 94; 109 | 7.22 | 62; 72; 113 | 10.75 | 22; 77; 101 | 3.93 | 79; 103; 129 | 23.38 | 7; 41; 109 | 6.03 |
| 25; 70; 113 | 8.66 | 6; 25; 109 | 7.22 | 63; 113; 135 | 10.75 | 61; 75; 101 | 3.93 | 79; 103; 132 | 23.38 | 29; 109; 150 | 6.03 |
| 113; 126; 131 | 8.66 | 8; 83; 109 | 7.22 | 71; 87; 113 | 10.75 | 7; 63; 101 | 3.93 | 66; 88; 103 | 23.38 | 6; 63; 109 | 6.03 |
| 29; 64; 113 | 8.66 | 8; 109; 132 | 7.22 | 35; 101; 113 | 10.75 | 29; 94; 101 | 3.93 | 101; 103; 143 | 23.38 | 2; 109; 114 | 6.03 |
| 84; 113; 131 | 8.66 | 28; 94; 109 | 7.22 | 42; 113; 120 | 10.75 | 7; 79; 101 | 3.93 | 79; 103; 142 | 23.38 | 31; 36; 114 | 6.03 |
| 6; 53; 113 | 8.66 | 38; 101; 109 | 7.22 | 71; 86; 138 | 10.75 | 18; 77; 101 | 3.93 | 97; 103; 135 | 23.38 | 25; 109; 150 | 6.03 |
| 10; 86; 113 | 8.66 | 5; 94; 109 | 7.22 | 56; 113; 126 | 10.75 | 7; 26; 101 | 3.93 | 68; 73; 103 | 23.38 | 72; 73; 109 | 6.03 |
| 29; 83; 113 | 8.66 | 81; 94; 109 | 7.22 | 1; 113; 150 | 10.75 | 101; 107; 129 | 3.93 | 66; 84; 103 | 23.38 | 45; 109; 114 | 6.03 |
| 64; 84; 113 | 8.66 | 20; 62; 109 | 7.22 | 22; 71; 113 | 10.75 | 29; 74; 101 | 3.93 | 48; 76; 103 | 23.38 | 55; 78; 109 | 6.03 |
| 62; 79; 113 | 8.66 | 64; 89; 109 | 7.22 | 56; 113; 150 | 10.75 | 7; 93; 101 | 3.93 | 66; 83; 103 | 23.38 | 14; 64; 109 | 6.03 |
| 53; 74; 113 | 8.66 | 8; 67; 109 | 7.22 | 10; 71; 113 | 10.75 | 29; 75; 101 | 3.93 | 57; 103; 120 | 23.38 | 41; 73; 109 | 6.03 |
| 33; 113; 131 | 8.66 | 25; 94; 149 | 7.22 | 56; 114; 120 | 10.75 | 62; 102; 129 | 3.93 | 67; 103; 143 | 23.38 | 6; 94; 109 | 6.03 |
| 53; 73; 113 | 8.66 | 25; 94; 148 | 7.22 | 75; 113; 150 | 10.75 | 29; 77; 101 | 3.93 | 42; 66; 103 | 23.38 | 44; 109; 114 | 6.03 |
| 63; 65; 113 | 8.66 | 8; 18; 109 | 7.22 | 70; 83; 113 | 10.75 | 62; 101; 129 | 3.93 | 73; 83; 103 | 23.38 | 70; 99; 109 | 6.03 |
| 64; 86; 113 | 8.66 | 25; 38; 94 | 7.22 | 1; 113; 135 | 10.75 | 29; 79; 101 | 3.93 | 66; 103; 132 | 23.38 | 6; 109; 114 | 6.03 |
| 29; 114; 120 | 8.66 | 25; 94; 134 | 7.22 | 7; 63; 113 | 10.75 | 88; 101; 129 | 3.93 | 66; 103; 133 | 23.38 | 7; 73; 109 | 6.03 |
| 63; 64; 113 | 8.66 | 25; 68; 109 | 7.22 | 70; 75; 113 | 10.75 | 7; 88; 101 | 3.93 | 66; 103; 134 | 23.38 | 70; 84; 109 | 6.03 |
| 18; 62; 113 | 8.66 | 63; 71; 109 | 7.22 | 70; 113; 150 | 10.75 | 44; 93; 101 | 3.93 | 54; 89; 103 | 23.38 | 26; 109; 114 | 6.03 |
| 62; 68; 113 | 8.66 | 101; 109; 132 | 7.22 | 38; 62; 113 | 10.75 | 29; 82; 101 | 3.93 | 10; 29; 103 | 23.38 | 63; 73; 109 | 6.03 |
| 88; 113; 131 | 8.66 | 63; 85; 109 | 7.22 | 10; 113; 120 | 10.75 | 7; 86; 101 | 3.93 | 63; 83; 103 | 23.38 | 6; 108; 109 | 6.03 |
| 70; 113; 131 | 8.66 | 63; 74; 109 | 7.22 | 56; 70; 113 | 10.75 | 44; 92; 101 | 3.93 | 21; 103; 129 | 23.38 | 70; 72; 109 | 6.03 |
| 22; 113; 120 | 8.66 | 8; 109; 120 | 7.22 | 22; 62; 113 | 10.75 | 29; 83; 101 | 3.93 | 72; 73; 103 | 23.38 | 14; 70; 109 | 6.03 |
| 69; 113; 131 | 8.66 | 18; 109; 132 | 7.22 | 101; 113; 131 | 10.75 | 7; 25; 101 | 3.93 | 66; 103; 142 | 23.38 | 14; 69; 109 | 6.03 |
| 4; 113; 131 | 8.66 | 25; 94; 147 | 7.22 | 101; 113; 132 | 10.75 | 29; 84; 101 | 3.93 | 57; 85; 103 | 23.38 | 6; 36; 109 | 6.03 |
| 62; 83; 113 | 8.66 | 10; 20; 109 | 7.22 | 6; 63; 113 | 10.75 | 29; 85; 101 | 3.93 | 29; 45; 103 | 23.38 | 55; 109; 150 | 6.03 |
| 21; 113; 134 | 8.66 | 25; 94; 119 | 7.22 | 101; 113; 134 | 10.75 | 7; 84; 101 | 3.93 | 10; 68; 103 | 23.38 | 26; 70; 109 | 6.03 |
| 1; 113; 126 | 8.66 | 89; 109; 129 | 7.22 | 56; 63; 113 | 10.75 | 7; 83; 101 | 3.93 | 33; 38; 103 | 23.38 | 26; 69; 109 | 6.03 |

| ELF3_AGBL2 positive cells | | ENS glia | | ENS neurons | | Endocardial cells | | Epicardial fat cells | | Erythroblasts | |
|---|---|---|---|---|---|---|---|---|---|---|---|
| Clusters | Score | Clusters | Score | Clusters | Score | Clusters | Score | Clusters | Score | Clusters | Score |
| 53; 113; 126 | 12.22 | 36; 77; 148 | 3.19 | 62; 100; 119 | 6.00 | 21; 121; 124 | 11.88 | 39; 49; 115 | 3.30 | 25; 28; 134 | 3.31 |
| 62; 113; 150 | 12.22 | 65; 93; 100 | 3.19 | 35; 87; 100 | 4.86 | 9; 113; 124 | 10.54 | 39; 50; 115 | 3.30 | 25; 53; 138 | 3.31 |
| 29; 71; 138 | 10.75 | 36; 77; 85 | 3.19 | 64; 89; 100 | 4.86 | 44; 121; 124 | 10.54 | 36; 39; 115 | 3.30 | 32; 130; 151 | 3.31 |
| 1; 83; 113 | 10.75 | 26; 38; 141 | 3.19 | 64; 90; 100 | 4.86 | 54; 113; 124 | 10.54 | 39; 62; 115 | 3.30 | 11; 28; 66 | 3.31 |
| 62; 75; 113 | 10.75 | 85; 100; 101 | 3.19 | 35; 68; 100 | 4.86 | 7; 124; 133 | 10.54 | 39; 115; 137 | 3.30 | 25; 83; 134 | 3.31 |
| 62; 113; 138 | 10.75 | 26; 66; 77 | 3.19 | 62; 85; 100 | 4.86 | 109; 123; 124 | 10.54 | 39; 64; 115 | 3.30 | 64; 67; 88 | 3.31 |
| 53; 101; 113 | 10.75 | 67; 96; 120 | 3.19 | 87; 100; 147 | 4.86 | 91; 121; 124 | 10.54 | 19; 39; 113 | 3.30 | 11; 62; 66 | 3.31 |
| 63; 65; 113 | 10.75 | 26; 77; 120 | 3.19 | 64; 97; 100 | 4.86 | 1; 121; 124 | 10.54 | 9; 19; 113 | 3.30 | 11; 64; 66 | 3.31 |
| 36; 101; 113 | 10.75 | 19; 76; 141 | 3.19 | 5; 35; 100 | 4.86 | 22; 123; 124 | 10.54 | 39; 107; 115 | 3.30 | 25; 27; 134 | 3.31 |
| 62; 93; 113 | 10.75 | 45; 98; 146 | 3.19 | 43; 87; 100 | 4.86 | 90; 120; 124 | 10.54 | 50; 105; 115 | 3.30 | 25; 69; 134 | 3.31 |
| 29; 65; 113 | 10.75 | 28; 67; 100 | 3.19 | 6; 100; 147 | 4.86 | 82; 88; 124 | 10.54 | 49; 115; 125 | 2.16 | 11; 28; 62 | 3.31 |
| 62; 84; 113 | 10.75 | 19; 77; 141 | 3.19 | 5; 97; 100 | 4.86 | 35; 81; 124 | 10.54 | 19; 92; 142 | 2.16 | 24; 96; 134 | 3.31 |
| 62; 83; 113 | 10.75 | 1; 26; 119 | 3.19 | 62; 87; 100 | 4.86 | 7; 121; 124 | 10.54 | 56; 105; 115 | 2.16 | 64; 67; 93 | 3.31 |
| 68; 75; 113 | 10.75 | 26; 66; 148 | 3.19 | 35; 70; 100 | 4.86 | 45; 121; 124 | 10.54 | 49; 115; 123 | 2.16 | 32; 64; 87 | 3.31 |
| 62; 81; 113 | 10.75 | 26; 71; 100 | 3.19 | 42; 97; 100 | 4.86 | 82; 124; 133 | 10.54 | 6; 50; 115 | 2.16 | 25; 78; 134 | 3.31 |
| 53; 113; 150 | 10.75 | 49; 79; 141 | 3.19 | 52; 81; 100 | 4.86 | 8; 121; 124 | 10.54 | 17; 19; 113 | 2.16 | 7; 135; 137 | 2.34 |
| 62; 79; 113 | 10.75 | 49; 81; 141 | 3.19 | 62; 83; 100 | 4.86 | 4; 121; 124 | 10.54 | 83; 139; 141 | 2.16 | 21; 135; 137 | 2.34 |
| 63; 73; 113 | 10.75 | 36; 101; 149 | 3.19 | 5; 100; 119 | 4.86 | 67; 124; 132 | 9.27 | 34; 50; 115 | 2.16 | 45; 64; 132 | 2.34 |
| 63; 68; 113 | 10.75 | 26; 72; 148 | 3.19 | 62; 82; 100 | 4.86 | 88; 124; 129 | 9.27 | 19; 92; 138 | 2.16 | 5; 96; 132 | 2.34 |
| 63; 88; 138 | 10.75 | 26; 72; 149 | 3.19 | 52; 100; 147 | 4.86 | 35; 124; 138 | 9.27 | 76; 120; 137 | 2.16 | 7; 64; 67 | 2.34 |
| 66; 74; 113 | 10.75 | 49; 119; 141 | 3.19 | 35; 82; 100 | 4.86 | 35; 124; 133 | 9.27 | 113; 115; 123 | 2.16 | 45; 64; 130 | 2.34 |
| 53; 65; 113 | 10.75 | 73; 84; 150 | 3.19 | 62; 100; 126 | 4.86 | 75; 124; 138 | 9.27 | 49; 113; 143 | 2.16 | 57; 66; 138 | 2.34 |
| 1; 65; 113 | 10.75 | 19; 126; 141 | 3.19 | 65; 100; 147 | 4.86 | 7; 67; 124 | 9.27 | 9; 87; 143 | 2.16 | 44; 66; 132 | 2.34 |
| 53; 68; 113 | 10.75 | 28; 36; 101 | 3.19 | 62; 100; 147 | 4.86 | 65; 113; 124 | 9.27 | 63; 88; 115 | 2.16 | 38; 57; 138 | 2.34 |
| 44; 113; 131 | 10.75 | 36; 83; 148 | 3.19 | 26; 68; 82 | 4.86 | 10; 121; 124 | 9.27 | 76; 120; 139 | 2.16 | 77; 95; 136 | 2.34 |
| 79; 113; 131 | 10.75 | 26; 67; 89 | 3.19 | 38; 62; 100 | 4.86 | 67; 124; 138 | 9.27 | 76; 121; 137 | 2.16 | 28; 49; 134 | 2.34 |
| 63; 100; 113 | 10.75 | 36; 120; 141 | 3.19 | 62; 84; 100 | 4.86 | 19; 121; 124 | 9.27 | 9; 92; 125 | 2.16 | 57; 66; 139 | 2.34 |
| 53; 71; 138 | 10.75 | 84; 100; 101 | 3.19 | 6; 35; 100 | 4.86 | 22; 124; 131 | 9.27 | 45; 50; 115 | 2.16 | 25; 44; 66 | 2.34 |
| 63; 93; 113 | 10.75 | 7; 36; 141 | 3.19 | 26; 68; 75 | 4.86 | 84; 88; 124 | 9.27 | 9; 88; 119 | 2.16 | 28; 49; 101 | 2.34 |
| 53; 81; 113 | 10.75 | 22; 64; 141 | 3.19 | 6; 51; 100 | 4.86 | 22; 124; 138 | 9.27 | 105; 113; 137 | 2.16 | 9; 32; 119 | 2.34 |
| 63; 70; 113 | 10.75 | 26; 94; 150 | 3.19 | 26; 68; 83 | 4.86 | 88; 124; 133 | 9.27 | 39; 56; 115 | 2.16 | 10; 76; 134 | 2.34 |
| 63; 87; 113 | 10.75 | 26; 94; 149 | 3.19 | 51; 87; 100 | 4.86 | 53; 93; 124 | 9.27 | 92; 129; 143 | 2.16 | 53; 62; 138 | 2.34 |
| 53; 83; 113 | 10.75 | 26; 94; 148 | 3.19 | 25; 87; 100 | 4.86 | 82; 84; 124 | 9.27 | 56; 121; 129 | 2.16 | 24; 132; 135 | 2.34 |
| 63; 85; 113 | 10.75 | 64; 98; 120 | 3.19 | 42; 100; 118 | 4.86 | 81; 121; 124 | 9.27 | 9; 92; 129 | 2.16 | 46; 96; 130 | 2.34 |
| 63; 84; 113 | 10.75 | 19; 84; 141 | 3.19 | 68; 87; 100 | 4.86 | 35; 124; 148 | 9.27 | 77; 86; 143 | 2.16 | 11; 28; 64 | 2.34 |
| 63; 83; 113 | 10.75 | 26; 93; 149 | 3.19 | 62; 64; 100 | 4.86 | 35; 124; 149 | 9.27 | 9; 96; 114 | 2.16 | 57; 64; 139 | 2.34 |
| 63; 81; 113 | 10.75 | 45; 67; 120 | 3.19 | 52; 62; 100 | 4.86 | 103; 121; 124 | 9.27 | 92; 143; 150 | 2.16 | 11; 61; 64 | 2.34 |
| 63; 79; 113 | 10.75 | 62; 67; 100 | 3.19 | 84; 87; 100 | 4.86 | 88; 124; 148 | 9.27 | 92; 143; 148 | 2.16 | 11; 61; 66 | 2.34 |
| 63; 75; 113 | 10.75 | 92; 100; 120 | 3.19 | 35; 65; 100 | 4.86 | 114; 116; 124 | 9.27 | 23; 121; 141 | 2.16 | 57; 64; 130 | 2.34 |
| 63; 74; 138 | 10.75 | 27; 83; 100 | 3.19 | 64; 100; 147 | 4.86 | 86; 124; 138 | 9.27 | 77; 92; 143 | 2.16 | 82; 96; 135 | 2.34 |
| 63; 74; 113 | 10.75 | 26; 88; 100 | 3.19 | 62; 97; 100 | 4.86 | 89; 124; 138 | 9.27 | 86; 139; 141 | 2.16 | 53; 57; 138 | 2.34 |
| 62; 73; 113 | 10.75 | 45; 67; 146 | 3.19 | 41; 62; 100 | 4.86 | 85; 121; 124 | 9.27 | 39; 61; 115 | 2.16 | 6; 11; 64 | 2.34 |
| 62; 74; 113 | 10.75 | 64; 92; 149 | 3.19 | 51; 81; 100 | 4.86 | 10; 124; 133 | 9.27 | 9; 97; 114 | 2.16 | 6; 135; 137 | 2.34 |
| 63; 113; 135 | 10.75 | 26; 89; 100 | 3.19 | 5; 100; 112 | 4.86 | 89; 124; 133 | 9.27 | 77; 86; 114 | 2.16 | 42; 61; 67 | 2.34 |
| 71; 87; 113 | 10.75 | 77; 100; 101 | 3.19 | 62; 89; 100 | 4.86 | 79; 121; 124 | 9.27 | 29; 71; 116 | 2.16 | 96; 126; 132 | 2.34 |
| 42; 113; 120 | 10.75 | 98; 126; 142 | 3.19 | 62; 90; 100 | 4.86 | 89; 123; 124 | 9.27 | 39; 61; 137 | 2.16 | 25; 83; 84 | 2.34 |
| 1; 113; 150 | 10.75 | 7; 26; 148 | 3.19 | 62; 70; 100 | 4.86 | 123; 124; 147 | 9.27 | 17; 49; 113 | 2.16 | 33; 66; 88 | 2.34 |
| 56; 113; 150 | 10.75 | 64; 88; 141 | 3.19 | 6; 62; 100 | 4.86 | 70; 121; 124 | 9.27 | 19; 29; 121 | 2.16 | 32; 135; 151 | 2.34 |
| 75; 113; 150 | 10.75 | 26; 84; 149 | 3.19 | 51; 62; 100 | 4.86 | 49; 124; 138 | 9.27 | 19; 29; 123 | 2.16 | 30; 90; 137 | 2.34 |
| 70; 83; 113 | 10.75 | 19; 82; 141 | 3.19 | 62; 68; 100 | 4.86 | 43; 121; 124 | 9.27 | 9; 92; 150 | 2.16 | 28; 49; 80 | 2.34 |
| 1; 113; 135 | 10.75 | 26; 82; 149 | 3.19 | 62; 98; 100 | 4.86 | 123; 124; 131 | 9.27 | 17; 19; 123 | 2.16 | 37; 44; 66 | 2.34 |
| 70; 75; 113 | 10.75 | 87; 100; 120 | 3.19 | 42; 69; 100 | 3.81 | 85; 120; 124 | 9.27 | 29; 72; 138 | 2.16 | 7; 137; 151 | 2.34 |
| 70; 113; 150 | 10.75 | 65; 107; 141 | 3.19 | 100; 118; 142 | 3.81 | 65; 121; 124 | 9.27 | 63; 71; 116 | 2.16 | 29; 57; 138 | 2.34 |
| 56; 70; 113 | 10.75 | 26; 79; 120 | 3.19 | 6; 68; 100 | 3.81 | 7; 69; 124 | 9.27 | 72; 75; 138 | 2.16 | 25; 82; 134 | 2.34 |
| 101; 113; 138 | 10.75 | 26; 79; 149 | 3.19 | 28; 81; 100 | 3.81 | 88; 124; 138 | 9.27 | 105; 113; 115 | 2.16 | 8; 25; 69 | 2.34 |
| 25; 63; 113 | 10.75 | 26; 100; 120 | 3.19 | 65; 100; 149 | 3.81 | 7; 70; 124 | 9.27 | 56; 115; 123 | 2.16 | 62; 69; 88 | 2.34 |
| 75; 79; 113 | 10.75 | 36; 67; 100 | 3.19 | 6; 73; 100 | 3.81 | 88; 124; 149 | 9.27 | 84; 92; 143 | 2.16 | 44; 135; 137 | 2.34 |
| 75; 83; 113 | 10.75 | 4; 67; 114 | 3.19 | 43; 84; 100 | 3.81 | 99; 124; 131 | 9.27 | 9; 79; 92 | 2.16 | 25; 74; 134 | 2.34 |
| 56; 79; 113 | 10.75 | 4; 67; 120 | 3.19 | 70; 100; 150 | 3.81 | 74; 82; 124 | 9.27 | 6; 39; 115 | 2.16 | 64; 132; 150 | 2.34 |
| 70; 71; 138 | 10.75 | 64; 108; 141 | 3.19 | 62; 100; 116 | 3.81 | 86; 124; 133 | 9.27 | 86; 115; 123 | 2.16 | 57; 91; 132 | 2.34 |
| 62; 70; 113 | 10.75 | 26; 81; 88 | 3.19 | 81; 100; 150 | 3.81 | 75; 121; 124 | 9.27 | 113; 120; 142 | 2.16 | 6; 124; 132 | 2.34 |
| 113; 131; 150 | 10.75 | 100; 101; 148 | 3.19 | 70; 100; 147 | 3.81 | 67; 88; 124 | 9.27 | 63; 105; 115 | 2.16 | 34; 64; 96 | 2.34 |
| 1; 44; 113 | 10.75 | 100; 101; 146 | 3.19 | 70; 100; 146 | 3.81 | 90; 121; 124 | 9.27 | 49; 97; 123 | 2.16 | 28; 95; 136 | 2.34 |
| 49; 114; 120 | 10.75 | 100; 101; 119 | 3.19 | 62; 100; 118 | 3.81 | 67; 111; 124 | 9.27 | 63; 107; 123 | 2.16 | 26; 57; 138 | 2.34 |
| 54; 101; 113 | 10.75 | 38; 70; 141 | 3.19 | 38; 70; 100 | 3.81 | 7; 124; 138 | 9.27 | 92; 115; 139 | 2.16 | 35; 64; 67 | 2.34 |
| 25; 101; 113 | 10.75 | 26; 82; 148 | 3.19 | 74; 100; 147 | 3.81 | 48; 124; 138 | 9.27 | 10; 115; 116 | 2.16 | 34; 64; 66 | 2.34 |
| 83; 113; 131 | 10.75 | 26; 120; 149 | 3.19 | 28; 82; 100 | 3.81 | 60; 113; 124 | 9.27 | 10; 115; 123 | 2.16 | 25; 49; 67 | 2.34 |
| 55; 70; 113 | 10.75 | 19; 119; 141 | 3.19 | 6; 72; 100 | 3.81 | 17; 67; 124 | 9.27 | 76; 86; 138 | 2.16 | 24; 32; 67 | 2.34 |
| 1; 25; 113 | 10.75 | 64; 71; 98 | 3.19 | 38; 72; 100 | 3.81 | 26; 121; 124 | 9.27 | 113; 122; 140 | 2.16 | 5; 57; 138 | 2.34 |
| 62; 65; 113 | 10.75 | 82; 83; 150 | 3.19 | 28; 87; 100 | 3.81 | 72; 121; 124 | 9.27 | 113; 122; 141 | 2.16 | 66; 76; 131 | 2.34 |
| 62; 68; 113 | 10.75 | 26; 69; 94 | 3.19 | 100; 118; 133 | 3.81 | 9; 121; 124 | 9.27 | 49; 89; 113 | 2.16 | 18; 25; 67 | 2.34 |
| 7; 113; 150 | 10.75 | 17; 36; 141 | 3.19 | 38; 68; 100 | 3.81 | 84; 113; 124 | 9.27 | 10; 116; 123 | 2.16 | 19; 32; 137 | 2.34 |
| 36; 62; 113 | 10.75 | 36; 141; 142 | 3.19 | 100; 118; 150 | 3.81 | 121; 124; 129 | 9.27 | 10; 116; 133 | 2.16 | 78; 124; 132 | 2.34 |
| 10; 113; 148 | 10.75 | 26; 68; 149 | 3.19 | 6; 70; 100 | 3.81 | 9; 123; 124 | 9.27 | 113; 121; 141 | 2.16 | 19; 42; 137 | 2.34 |
| 10; 36; 113 | 10.75 | 68; 74; 77 | 2.25 | 100; 119; 123 | 3.81 | 85; 88; 124 | 9.27 | 14; 105; 115 | 2.16 | 57; 69; 138 | 2.34 |
| 36; 113; 126 | 10.75 | 5; 44; 141 | 2.25 | 38; 73; 100 | 3.81 | 7; 124; 148 | 9.27 | 23; 137; 141 | 2.16 | 25; 120; 134 | 2.34 |
| 36; 113; 151 | 10.75 | 64; 81; 107 | 2.25 | 43; 83; 100 | 3.81 | 60; 121; 124 | 9.27 | 10; 105; 115 | 2.16 | 8; 27; 96 | 2.34 |
| 36; 113; 138 | 10.75 | 26; 81; 100 | 2.25 | 100; 118; 126 | 3.81 | 48; 123; 124 | 9.27 | 49; 107; 123 | 2.16 | 29; 76; 134 | 2.34 |
| 36; 113; 134 | 10.75 | 38; 69; 84 | 2.25 | 74; 100; 118 | 3.81 | 29; 121; 124 | 9.27 | 121; 137; 141 | 2.16 | 64; 66; 92 | 2.34 |
| 36; 113; 131 | 10.75 | 53; 83; 150 | 2.25 | 100; 118; 123 | 3.81 | 120; 124; 149 | 9.27 | 105; 107; 115 | 2.16 | 55; 65; 131 | 2.34 |
| 29; 101; 113 | 10.75 | 4; 10; 141 | 2.25 | 6; 74; 100 | 3.81 | 70; 88; 124 | 9.27 | 121; 135; 137 | 2.16 | 57; 131; 133 | 2.34 |
| 53; 63; 113 | 10.75 | 68; 72; 141 | 2.25 | 38; 74; 100 | 3.81 | 88; 93; 124 | 9.27 | 9; 85; 92 | 2.16 | 57; 68; 138 | 2.34 |
| 65; 113; 131 | 10.75 | 62; 100; 101 | 2.25 | 74; 100; 119 | 3.81 | 34; 121; 124 | 9.27 | 113; 115; 141 | 2.16 | 25; 77; 134 | 2.34 |
| 63; 113; 150 | 10.75 | 27; 100; 150 | 2.25 | 38; 69; 100 | 3.81 | 67; 92; 124 | 9.27 | 17; 50; 123 | 2.16 | 84; 135; 137 | 2.34 |
| 44; 63; 113 | 10.75 | 36; 65; 71 | 2.25 | 100; 118; 149 | 3.81 | 44; 113; 124 | 9.27 | 76; 115; 123 | 2.16 | 34; 62; 96 | 2.34 |
| 64; 113; 150 | 10.75 | 29; 92; 141 | 2.25 | 81; 100; 133 | 3.81 | 13; 124; 129 | 9.27 | 49; 105; 115 | 2.16 | 32; 87; 130 | 2.34 |
| 29; 113; 131 | 10.75 | 64; 81; 101 | 2.25 | 6; 75; 100 | 3.81 | 121; 124; 140 | 9.27 | 121; 129; 150 | 2.16 | 64; 107; 134 | 2.34 |
| 29; 114; 120 | 10.75 | 26; 64; 95 | 2.25 | 62; 100; 102 | 3.81 | 121; 124; 141 | 9.27 | 9; 82; 138 | 2.16 | 32; 44; 137 | 2.34 |
| 44; 62; 113 | 10.75 | 36; 120; 134 | 2.25 | 6; 69; 100 | 3.81 | 121; 124; 142 | 9.27 | 39; 129; 137 | 2.16 | 64; 65; 131 | 2.34 |
| 28; 63; 113 | 10.75 | 62; 98; 148 | 2.25 | 62; 100; 123 | 3.81 | 121; 124; 143 | 9.27 | 50; 89; 115 | 2.16 | 55; 83; 134 | 2.34 |
| 43; 100; 113 | 10.75 | 68; 78; 80 | 2.25 | 5; 87; 100 | 3.81 | 98; 121; 124 | 9.27 | 36; 116; 123 | 2.16 | 57; 67; 130 | 2.34 |
| 63; 113; 138 | 10.75 | 68; 77; 150 | 2.25 | 74; 82; 100 | 3.81 | 114; 124; 131 | 9.27 | 50; 94; 115 | 2.16 | 90; 134; 137 | 2.34 |
| 1; 55; 113 | 10.75 | 26; 81; 129 | 2.25 | 28; 51; 100 | 3.81 | 90; 124; 138 | 9.27 | 50; 93; 115 | 2.16 | 44; 49; 67 | 2.34 |
| 1; 53; 113 | 10.75 | 26; 81; 120 | 2.25 | 82; 84; 100 | 3.81 | 67; 123; 124 | 9.27 | 50; 92; 123 | 2.16 | 66; 69; 132 | 2.34 |
| 72; 83; 113 | 10.75 | 45; 87; 100 | 2.25 | 82; 83; 100 | 3.81 | 99; 121; 124 | 9.27 | 50; 92; 115 | 2.16 | 84; 96; 132 | 2.34 |
| 34; 75; 113 | 9.37 | 36; 64; 120 | 2.25 | 100; 126; 147 | 3.81 | 82; 120; 124 | 9.27 | 50; 85; 115 | 2.16 | 64; 72; 134 | 2.34 |
| 1; 28; 113 | 9.37 | 26; 64; 89 | 2.25 | 65; 84; 100 | 3.81 | 35; 69; 124 | 9.27 | 6; 61; 121 | 2.16 | 64; 134; 135 | 2.34 |
| 25; 70; 113 | 9.37 | 62; 100; 119 | 2.25 | 5; 86; 100 | 3.81 | 90; 123; 124 | 9.27 | 19; 116; 121 | 2.16 | 6; 30; 137 | 2.34 |
| 10; 113; 150 | 9.37 | 36; 64; 140 | 2.25 | 65; 87; 100 | 3.81 | 21; 67; 124 | 9.27 | 50; 88; 115 | 2.16 | 45; 64; 93 | 2.34 |
| 71; 93; 114 | 9.37 | 36; 64; 141 | 2.25 | 43; 81; 100 | 3.81 | 82; 121; 124 | 9.27 | 83; 105; 115 | 2.16 | 6; 66; 132 | 2.34 |

| Excitatory neurons | | Extravillous trophoblasts | | Ganglion cells | | Goblet cells | | Granule neurons | | Hematopoietic stem cells | |
|---|---|---|---|---|---|---|---|---|---|---|---|
| Clusters | Score | Clusters | Score | Clusters | Score | Clusters | Score | Clusters | Score | Clusters | Score |
| 27; 57; 97 | 4.60 | 84; 94; 114 | 5.73 | 43; 59; 127 | 3.57 | 68; 71; 99 | 3.45 | 26; 84; 102 | 3.60 | 17; 80; 132 | 8.90 |
| 40; 98; 138 | 4.60 | 81; 94; 114 | 5.73 | 26; 59; 127 | 3.57 | 34; 55; 77 | 3.45 | 27; 62; 135 | 3.60 | 17; 76; 129 | 7.41 |
| 27; 74; 97 | 4.60 | 37; 94; 114 | 4.35 | 59; 92; 127 | 3.57 | 19; 59; 104 | 2.45 | 28; 49; 102 | 3.60 | 49; 70; 147 | 7.41 |
| 27; 75; 147 | 3.30 | 37; 94; 148 | 4.35 | 95; 124; 128 | 3.57 | 9; 34; 112 | 2.45 | 69; 90; 103 | 3.60 | 13; 66; 85 | 7.41 |
| 39; 73; 97 | 3.30 | 36; 38; 81 | 4.35 | 8; 59; 127 | 3.57 | 20; 85; 103 | 2.45 | 29; 63; 77 | 3.60 | 49; 76; 80 | 7.41 |
| 27; 75; 103 | 3.30 | 14; 98; 114 | 3.12 | 32; 59; 128 | 3.57 | 71; 85; 99 | 2.45 | 26; 29; 102 | 3.60 | 13; 66; 87 | 7.41 |
| 27; 75; 97 | 3.30 | 53; 94; 114 | 3.12 | 59; 94; 127 | 3.57 | 85; 103; 108 | 2.45 | 27; 35; 135 | 3.60 | 17; 62; 80 | 7.41 |
| 40; 89; 98 | 3.30 | 76; 81; 108 | 3.12 | 59; 125; 127 | 3.57 | 19; 122; 125 | 2.45 | 13; 28; 102 | 3.60 | 49; 79; 148 | 7.41 |
| 40; 89; 97 | 3.30 | 84; 94; 149 | 3.12 | 9; 122; 128 | 3.57 | 9; 99; 147 | 2.45 | 19; 27; 135 | 3.60 | 49; 78; 149 | 7.41 |
| 24; 83; 97 | 3.30 | 26; 114; 149 | 3.12 | 59; 127; 146 | 3.57 | 62; 74; 100 | 2.45 | 8; 98; 102 | 3.60 | 49; 129; 148 | 7.41 |
| 39; 74; 98 | 3.30 | 38; 81; 85 | 3.12 | 128; 136; 143 | 3.57 | 8; 32; 109 | 2.45 | 28; 102; 141 | 3.60 | 49; 78; 147 | 7.41 |
| 39; 74; 97 | 3.30 | 38; 81; 92 | 3.12 | 12; 59; 128 | 3.57 | 34; 57; 83 | 2.45 | 67; 90; 103 | 3.60 | 17; 66; 80 | 7.41 |
| 24; 84; 97 | 3.30 | 52; 86; 114 | 3.12 | 59; 127; 148 | 3.57 | 62; 72; 100 | 2.45 | 26; 90; 102 | 3.60 | 49; 147; 149 | 7.41 |
| 26; 55; 97 | 3.30 | 1; 97; 114 | 3.12 | 59; 80; 127 | 3.57 | 18; 84; 133 | 2.45 | 27; 44; 131 | 3.60 | 13; 66; 78 | 7.41 |
| 27; 73; 103 | 3.30 | 72; 81; 84 | 3.12 | 43; 56; 118 | 3.57 | 36; 120; 133 | 2.45 | 81; 90; 103 | 3.60 | 17; 75; 80 | 7.41 |
| 39; 70; 98 | 3.30 | 94; 109; 114 | 3.12 | 20; 59; 127 | 3.57 | 18; 69; 108 | 2.45 | 21; 34; 141 | 3.60 | 13; 69; 150 | 7.41 |
| 39; 70; 97 | 3.30 | 16; 36; 81 | 3.12 | 25; 26; 97 | 3.57 | 34; 55; 147 | 2.45 | 26; 81; 102 | 3.60 | 49; 66; 77 | 7.41 |
| 28; 47; 98 | 3.30 | 37; 52; 114 | 3.12 | 1; 59; 127 | 3.57 | 32; 122; 134 | 2.45 | 26; 28; 102 | 3.60 | 13; 49; 148 | 7.41 |
| 27; 74; 147 | 3.30 | 52; 73; 114 | 3.12 | 28; 51; 97 | 3.57 | 31; 62; 81 | 2.45 | 27; 102; 131 | 3.60 | 49; 77; 148 | 7.41 |
| 27; 74; 103 | 3.30 | 14; 92; 114 | 3.12 | 59; 126; 127 | 3.57 | 32; 99; 122 | 2.45 | 24; 99; 102 | 3.60 | 17; 75; 147 | 7.41 |
| 27; 74; 98 | 3.30 | 16; 81; 149 | 3.12 | 43; 100; 127 | 3.57 | 55; 126; 147 | 2.45 | 24; 50; 102 | 3.60 | 13; 64; 80 | 7.41 |
| 27; 73; 147 | 3.30 | 33; 52; 114 | 3.12 | 32; 40; 128 | 3.57 | 34; 55; 149 | 2.45 | 27; 40; 135 | 3.60 | 49; 69; 77 | 7.41 |
| 40; 89; 138 | 3.30 | 37; 81; 92 | 3.12 | 59; 65; 127 | 3.57 | 31; 34; 105 | 2.45 | 12; 27; 102 | 3.60 | 13; 66; 77 | 6.03 |
| 37; 39; 98 | 3.30 | 85; 94; 114 | 3.12 | 9; 100; 128 | 3.57 | 31; 62; 83 | 2.45 | 28; 99; 102 | 3.60 | 13; 49; 69 | 6.03 |
| 24; 79; 97 | 3.30 | 14; 56; 114 | 3.12 | 84; 127; 135 | 3.57 | 31; 34; 103 | 2.45 | 29; 34; 149 | 3.60 | 49; 79; 147 | 6.03 |
| 10; 27; 102 | 3.30 | 52; 72; 114 | 3.12 | 59; 119; 127 | 3.57 | 55; 77; 147 | 2.45 | 27; 37; 78 | 3.60 | 49; 129; 147 | 6.03 |
| 27; 79; 147 | 3.30 | 36; 96; 114 | 3.12 | 59; 127; 134 | 3.57 | 55; 79; 147 | 2.45 | 8; 78; 102 | 3.60 | 17; 56; 69 | 6.03 |
| 27; 79; 107 | 3.30 | 97; 108; 114 | 3.12 | 59; 127; 136 | 3.57 | 36; 120; 125 | 2.45 | 8; 26; 102 | 3.60 | 49; 80; 81 | 6.03 |
| 27; 79; 103 | 3.30 | 48; 81; 148 | 3.12 | 25; 102; 127 | 3.57 | 31; 62; 84 | 2.45 | 26; 61; 102 | 3.60 | 49; 72; 80 | 6.03 |
| 27; 79; 102 | 3.30 | 36; 81; 150 | 3.12 | 89; 127; 135 | 3.57 | 24; 62; 122 | 2.45 | 27; 40; 102 | 3.60 | 17; 80; 147 | 6.03 |
| 27; 79; 97 | 3.30 | 52; 75; 114 | 3.12 | 17; 59; 127 | 3.57 | 23; 122; 135 | 2.45 | 26; 69; 102 | 3.60 | 17; 80; 150 | 6.03 |
| 28; 39; 89 | 3.30 | 52; 74; 114 | 3.12 | 59; 99; 127 | 3.57 | 24; 62; 121 | 2.45 | 7; 26; 102 | 3.60 | 17; 80; 129 | 6.03 |
| 24; 81; 97 | 3.30 | 14; 52; 114 | 3.12 | 51; 59; 127 | 3.57 | 31; 62; 74 | 2.45 | 84; 90; 103 | 3.60 | 49; 76; 119 | 6.03 |
| 40; 90; 98 | 3.30 | 38; 81; 149 | 3.12 | 40; 128; 136 | 3.57 | 34; 57; 84 | 2.45 | 26; 74; 102 | 3.60 | 13; 66; 84 | 6.03 |
| 40; 90; 97 | 3.30 | 52; 114; 129 | 3.12 | 59; 82; 127 | 3.57 | 71; 74; 100 | 2.45 | 27; 77; 131 | 3.60 | 49; 95; 145 | 6.03 |
| 39; 69; 97 | 3.30 | 36; 79; 81 | 3.12 | 59; 95; 128 | 3.57 | 19; 99; 125 | 2.45 | 6; 26; 102 | 3.60 | 13; 66; 80 | 6.03 |
| 39; 75; 97 | 3.30 | 14; 37; 114 | 3.12 | 12; 51; 128 | 3.57 | 84; 120; 125 | 2.45 | 21; 43; 78 | 3.60 | 14; 66; 77 | 6.03 |
| 28; 39; 97 | 3.30 | 37; 114; 129 | 3.12 | 59; 77; 127 | 3.57 | 32; 63; 121 | 2.45 | 21; 43; 69 | 3.60 | 49; 80; 82 | 6.03 |
| 39; 75; 98 | 3.30 | 70; 94; 114 | 3.12 | 61; 127; 135 | 3.57 | 43; 122; 124 | 2.45 | 23; 102; 141 | 3.60 | 66; 72; 92 | 6.03 |
| 12; 97; 113 | 3.30 | 37; 114; 143 | 3.12 | 44; 59; 127 | 3.57 | 75; 99; 129 | 2.45 | 28; 29; 134 | 3.60 | 17; 80; 135 | 6.03 |
| 39; 69; 98 | 3.30 | 81; 92; 109 | 3.12 | 5; 59; 127 | 3.57 | 18; 34; 68 | 2.45 | 53; 81; 98 | 3.60 | 77; 87; 135 | 6.03 |
| 24; 89; 147 | 3.30 | 101; 108; 114 | 3.12 | 28; 102; 127 | 3.57 | 18; 88; 133 | 2.45 | 18; 26; 102 | 3.60 | 49; 95; 148 | 6.03 |
| 24; 118; 127 | 3.30 | 26; 36; 114 | 3.12 | 97; 101; 118 | 3.57 | 62; 71; 100 | 2.45 | 26; 83; 102 | 3.60 | 49; 65; 80 | 6.03 |
| 24; 118; 137 | 3.30 | 56; 97; 113 | 3.12 | 44; 127; 135 | 3.57 | 28; 85; 103 | 2.45 | 12; 29; 61 | 3.60 | 49; 80; 97 | 6.03 |
| 24; 119; 133 | 3.30 | 1; 13; 114 | 3.12 | 9; 26; 97 | 3.57 | 34; 55; 129 | 2.45 | 27; 50; 125 | 3.60 | 56; 66; 77 | 6.03 |
| 24; 119; 138 | 3.30 | 81; 94; 149 | 3.12 | 17; 61; 127 | 3.57 | 70; 71; 125 | 2.45 | 26; 43; 77 | 3.60 | 13; 70; 87 | 6.03 |
| 27; 64; 98 | 3.30 | 81; 94; 150 | 3.12 | 52; 59; 127 | 3.57 | 31; 54; 120 | 2.45 | 21; 28; 142 | 3.60 | 18; 78; 129 | 6.03 |
| 21; 40; 119 | 3.30 | 1; 109; 114 | 3.12 | 59; 81; 127 | 3.57 | 34; 71; 147 | 2.45 | 29; 93; 146 | 3.60 | 17; 56; 148 | 6.03 |
| 21; 40; 98 | 3.30 | 94; 114; 118 | 3.12 | 25; 97; 135 | 3.57 | 34; 57; 98 | 2.45 | 24; 40; 102 | 3.60 | 18; 72; 80 | 6.03 |
| 6; 39; 97 | 3.30 | 64; 71; 97 | 3.12 | 40; 95; 128 | 3.57 | 8; 31; 120 | 2.45 | 27; 97; 102 | 3.60 | 17; 147; 150 | 6.03 |
| 27; 65; 103 | 3.30 | 35; 81; 84 | 3.12 | 43; 99; 127 | 3.57 | 31; 62; 63 | 2.45 | 27; 96; 102 | 3.60 | 17; 28; 77 | 6.03 |
| 12; 79; 113 | 3.30 | 36; 81; 93 | 3.12 | 32; 128; 143 | 3.57 | 31; 70; 71 | 2.45 | 24; 39; 102 | 3.60 | 18; 72; 78 | 6.03 |
| 24; 114; 127 | 3.30 | 15; 97; 114 | 3.12 | 10; 59; 127 | 3.57 | 99; 120; 129 | 2.45 | 26; 54; 102 | 3.60 | 17; 28; 80 | 6.03 |
| 28; 53; 103 | 3.30 | 20; 94; 148 | 3.12 | 95; 128; 143 | 2.66 | 34; 57; 89 | 2.45 | 27; 125; 131 | 3.60 | 17; 56; 119 | 6.03 |
| 28; 89; 97 | 3.30 | 37; 109; 113 | 3.12 | 7; 28; 97 | 2.66 | 62; 109; 122 | 2.45 | 28; 43; 77 | 3.60 | 49; 80; 95 | 6.03 |
| 21; 39; 97 | 3.30 | 81; 97; 108 | 3.12 | 95; 128; 142 | 2.66 | 30; 32; 100 | 2.45 | 21; 28; 64 | 3.60 | 17; 66; 77 | 6.03 |
| 27; 44; 103 | 3.30 | 81; 97; 109 | 3.12 | 95; 128; 133 | 2.66 | 31; 62; 68 | 2.45 | 27; 47; 102 | 3.60 | 28; 49; 119 | 6.03 |
| 28; 56; 97 | 3.30 | 81; 97; 113 | 3.12 | 95; 128; 146 | 2.66 | 31; 62; 69 | 2.45 | 29; 42; 141 | 3.60 | 49; 68; 77 | 6.03 |
| 28; 56; 98 | 3.30 | 16; 37; 149 | 3.12 | 9; 40; 128 | 2.66 | 68; 71; 125 | 2.45 | 1; 29; 102 | 3.60 | 18; 49; 77 | 6.03 |
| 27; 61; 103 | 3.30 | 36; 81; 86 | 3.12 | 7; 28; 118 | 2.66 | 31; 63; 133 | 2.45 | 21; 34; 43 | 3.60 | 49; 80; 83 | 6.03 |
| 27; 61; 102 | 3.30 | 36; 81; 85 | 3.12 | 31; 127; 129 | 2.66 | 31; 62; 70 | 2.45 | 13; 27; 102 | 3.60 | 49; 80; 84 | 6.03 |
| 27; 61; 98 | 3.30 | 35; 81; 149 | 3.12 | 95; 128; 129 | 2.66 | 5; 100; 120 | 2.45 | 28; 64; 102 | 3.60 | 66; 77; 86 | 6.03 |
| 34; 79; 97 | 3.30 | 35; 81; 148 | 3.12 | 95; 128; 150 | 2.66 | 63; 88; 133 | 2.45 | 8; 21; 119 | 3.60 | 17; 56; 80 | 6.03 |
| 23; 61; 97 | 3.30 | 94; 97; 114 | 3.12 | 44; 95; 128 | 2.66 | 34; 68; 92 | 2.45 | 12; 28; 141 | 3.60 | 49; 95; 119 | 6.03 |
| 27; 62; 103 | 3.30 | 81; 96; 109 | 3.12 | 95; 128; 134 | 2.66 | 36; 112; 133 | 2.45 | 26; 53; 102 | 3.60 | 49; 80; 85 | 6.03 |
| 39; 61; 98 | 3.30 | 94; 97; 109 | 3.12 | 29; 95; 128 | 2.66 | 18; 62; 100 | 2.45 | 24; 102; 127 | 3.60 | 17; 66; 72 | 6.03 |
| 39; 61; 97 | 3.30 | 94; 114; 148 | 3.12 | 24; 43; 97 | 2.66 | 90; 99; 129 | 2.45 | 29; 96; 149 | 3.60 | 49; 80; 88 | 6.03 |
| 27; 66; 97 | 3.30 | 29; 94; 114 | 3.12 | 93; 101; 118 | 2.66 | 80; 99; 126 | 2.45 | 28; 78; 102 | 3.60 | 49; 76; 78 | 6.03 |
| 27; 66; 103 | 3.30 | 59; 115; 121 | 3.12 | 1; 44; 118 | 2.66 | 31; 62; 120 | 2.45 | 29; 98; 102 | 3.60 | 49; 76; 77 | 6.03 |
| 24; 97; 150 | 3.30 | 36; 81; 82 | 3.12 | 95; 128; 135 | 2.66 | 32; 48; 131 | 2.45 | 21; 78; 135 | 3.60 | 35; 77; 135 | 6.03 |
| 27; 70; 103 | 3.30 | 6; 13; 114 | 3.12 | 32; 73; 128 | 2.66 | 82; 99; 126 | 2.45 | 90; 93; 103 | 3.60 | 49; 77; 79 | 6.03 |
| 27; 37; 103 | 3.30 | 81; 82; 108 | 3.12 | 95; 128; 132 | 2.66 | 70; 103; 129 | 2.45 | 12; 28; 145 | 3.60 | 49; 97; 135 | 6.03 |
| 24; 96; 97 | 3.30 | 7; 97; 114 | 3.12 | 95; 128; 136 | 2.66 | 18; 99; 125 | 2.45 | 28; 60; 102 | 3.60 | 49; 69; 148 | 6.03 |
| 24; 96; 98 | 3.30 | 52; 108; 114 | 3.12 | 95; 128; 151 | 2.66 | 64; 103; 129 | 2.45 | 9; 27; 134 | 3.60 | 17; 77; 126 | 6.03 |
| 27; 69; 103 | 3.30 | 56; 64; 114 | 3.12 | 44; 74; 127 | 2.66 | 31; 69; 71 | 2.45 | 9; 27; 78 | 3.60 | 17; 77; 132 | 6.03 |
| 24; 97; 114 | 3.30 | 13; 94; 114 | 3.12 | 9; 122; 127 | 2.66 | 25; 71; 100 | 2.45 | 21; 64; 84 | 3.60 | 49; 77; 119 | 6.03 |
| 28; 98; 116 | 3.30 | 1; 36; 114 | 3.12 | 26; 118; 149 | 2.66 | 55; 68; 147 | 2.45 | 29; 84; 96 | 3.60 | 49; 69; 147 | 6.03 |
| 24; 97; 147 | 3.30 | 38; 84; 148 | 3.12 | 29; 70; 97 | 2.66 | 69; 103; 129 | 2.45 | 22; 27; 102 | 3.60 | 17; 77; 134 | 6.03 |
| 24; 97; 148 | 3.30 | 7; 13; 114 | 3.12 | 43; 53; 118 | 2.66 | 18; 70; 108 | 2.45 | 12; 28; 43 | 3.60 | 17; 77; 135 | 6.03 |
| 27; 37; 98 | 3.30 | 36; 94; 114 | 3.12 | 6; 11; 97 | 2.66 | 31; 68; 103 | 2.45 | 26; 77; 102 | 3.60 | 49; 78; 119 | 6.03 |
| 28; 48; 102 | 3.30 | 71; 109; 114 | 3.12 | 32; 72; 128 | 2.66 | 20; 55; 103 | 2.45 | 27; 39; 131 | 3.60 | 13; 65; 78 | 6.03 |
| 20; 39; 97 | 3.30 | 30; 114; 143 | 3.12 | 5; 32; 128 | 2.66 | 62; 88; 100 | 2.45 | 26; 102; 150 | 3.60 | 17; 77; 147 | 6.03 |
| 9; 39; 98 | 3.30 | 56; 114; 129 | 3.12 | 60; 95; 127 | 2.66 | 8; 30; 100 | 2.45 | 28; 94; 131 | 3.60 | 17; 77; 150 | 6.03 |
| 40; 84; 98 | 3.30 | 81; 82; 84 | 3.12 | 60; 95; 128 | 2.66 | 25; 107; 132 | 2.45 | 21; 64; 81 | 3.60 | 49; 78; 129 | 6.03 |
| 17; 97; 138 | 3.30 | 36; 74; 81 | 3.12 | 17; 43; 127 | 2.66 | 19; 64; 104 | 2.45 | 28; 96; 102 | 3.60 | 13; 65; 87 | 6.03 |
| 6; 12; 113 | 3.30 | 53; 81; 108 | 3.12 | 9; 124; 128 | 2.66 | 27; 70; 103 | 2.45 | 12; 28; 61 | 3.60 | 17; 78; 80 | 6.03 |
| 12; 89; 113 | 3.30 | 13; 108; 114 | 3.12 | 37; 102; 118 | 2.66 | 5; 34; 92 | 2.45 | 26; 76; 102 | 3.60 | 17; 69; 77 | 6.03 |
| 28; 48; 118 | 3.30 | 36; 76; 94 | 3.12 | 9; 124; 127 | 2.66 | 19; 31; 100 | 2.45 | 27; 78; 102 | 3.60 | 49; 69; 119 | 6.03 |
| 28; 48; 119 | 3.30 | 81; 82; 86 | 3.12 | 43; 53; 97 | 2.66 | 19; 64; 91 | 2.45 | 8; 21; 81 | 2.56 | 17; 76; 80 | 6.03 |
| 39; 68; 97 | 3.30 | 13; 114; 140 | 3.12 | 31; 54; 127 | 2.66 | 69; 103; 108 | 2.45 | 29; 37; 95 | 2.56 | 18; 64; 80 | 6.03 |
| 28; 48; 138 | 3.30 | 28; 36; 94 | 3.12 | 40; 99; 127 | 2.66 | 31; 62; 138 | 2.45 | 12; 28; 37 | 2.56 | 49; 77; 126 | 6.03 |
| 24; 97; 149 | 3.30 | 52; 68; 114 | 3.12 | 9; 120; 125 | 2.66 | 31; 62; 148 | 2.45 | 27; 119; 135 | 2.56 | 49; 77; 129 | 6.03 |
| 24; 98; 102 | 3.30 | 29; 114; 142 | 3.12 | 32; 70; 128 | 2.66 | 31; 44; 62 | 2.45 | 8; 21; 78 | 2.56 | 49; 77; 149 | 6.03 |
| 24; 98; 103 | 3.30 | 36; 84; 147 | 3.12 | 59; 72; 127 | 2.66 | 5; 31; 71 | 2.45 | 47; 67; 78 | 2.56 | 49; 78; 80 | 6.03 |
| 12; 84; 113 | 3.30 | 94; 148; 149 | 3.12 | 46; 95; 127 | 2.66 | 79; 99; 129 | 2.45 | 21; 70; 149 | 2.56 | 49; 78; 81 | 6.03 |
| 12; 40; 114 | 3.30 | 94; 148; 150 | 3.12 | 46; 95; 128 | 2.66 | 62; 85; 103 | 2.45 | 27; 119; 141 | 2.56 | 49; 78; 82 | 6.03 |
| 12; 40; 117 | 3.30 | 81; 109; 113 | 3.12 | 59; 94; 128 | 2.66 | 34; 35; 69 | 2.45 | 9; 27; 149 | 2.56 | 49; 77; 146 | 6.03 |
| 12; 40; 118 | 3.30 | 13; 80; 114 | 3.12 | 22; 95; 128 | 2.66 | 20; 70; 103 | 2.45 | 8; 21; 103 | 2.56 | 49; 78; 84 | 6.03 |
| 12; 40; 119 | 3.30 | 13; 97; 113 | 3.12 | 15; 95; 128 | 2.66 | 62; 74; 133 | 2.45 | 8; 21; 118 | 2.56 | 49; 77; 145 | 6.03 |
| 12; 40; 127 | 3.30 | 52; 112; 114 | 3.12 | 59; 95; 127 | 2.66 | 68; 120; 125 | 2.45 | 24; 90; 102 | 2.56 | 49; 70; 80 | 6.03 |

| Hepatoblasts | | Horizontal cells | | IGFBP1_DKK1 positive cells | | Inhibitory interneurons | | Inhibitory neurons | | Intestinal epithelial cells | |
|---|---|---|---|---|---|---|---|---|---|---|---|
| Clusters | Score | Clusters | Score | Clusters | Score | Clusters | Score | Clusters | Score | Clusters | Score |
| 41; 43; 107 | 5.19 | 23; 85; 97 | 5.06 | 129; 143; 145 | 3.96 | 6; 94; 98 | 6.90 | 22; 81; 99 | 5.64 | 6; 43; 96 | 6.00 |
| 16; 41; 43 | 5.19 | 24; 88; 98 | 5.06 | 8; 114; 135 | 3.96 | 1; 79; 96 | 5.82 | 15; 22; 99 | 5.64 | 41; 43; 63 | 6.00 |
| 3; 29; 148 | 5.19 | 25; 28; 98 | 5.06 | 40; 54; 130 | 3.96 | 8; 81; 92 | 5.82 | 1; 15; 99 | 4.78 | 41; 43; 147 | 6.00 |
| 3; 84; 86 | 5.19 | 29; 98; 126 | 5.06 | 130; 142; 144 | 3.96 | 56; 68; 92 | 5.82 | 18; 89; 99 | 4.78 | 43; 55; 147 | 6.00 |
| 3; 29; 149 | 5.19 | 23; 88; 97 | 5.06 | 129; 133; 145 | 3.96 | 6; 26; 89 | 5.82 | 15; 24; 39 | 4.78 | 6; 43; 45 | 6.00 |
| 43; 44; 127 | 3.93 | 29; 84; 98 | 5.06 | 130; 141; 149 | 3.96 | 1; 84; 98 | 5.82 | 26; 56; 89 | 4.78 | 1; 43; 53 | 6.00 |
| 43; 68; 106 | 3.93 | 29; 82; 98 | 5.06 | 130; 141; 145 | 3.96 | 8; 37; 97 | 5.82 | 23; 76; 101 | 4.78 | 1; 43; 61 | 6.00 |
| 3; 61; 96 | 3.93 | 24; 98; 117 | 5.06 | 73; 129; 135 | 3.96 | 1; 79; 98 | 5.82 | 26; 49; 111 | 4.78 | 25; 43; 96 | 6.00 |
| 16; 20; 43 | 3.93 | 28; 29; 98 | 5.06 | 88; 129; 145 | 3.96 | 1; 96; 98 | 5.82 | 40; 105; 110 | 4.78 | 1; 25; 43 | 6.00 |
| 2; 43; 61 | 3.93 | 23; 62; 97 | 5.06 | 40; 62; 130 | 3.96 | 1; 83; 96 | 5.82 | 18; 86; 89 | 4.78 | 30; 43; 119 | 6.00 |
| 3; 61; 86 | 3.93 | 29; 85; 98 | 5.06 | 129; 135; 142 | 3.96 | 81; 89; 94 | 5.82 | 18; 34; 89 | 4.78 | 33; 43; 96 | 6.00 |
| 26; 43; 52 | 3.93 | 24; 61; 102 | 5.06 | 37; 129; 149 | 3.96 | 1; 83; 98 | 5.82 | 16; 39; 101 | 4.78 | 6; 35; 43 | 6.00 |
| 3; 61; 84 | 3.93 | 24; 61; 98 | 5.06 | 70; 129; 135 | 3.07 | 1; 56; 96 | 5.82 | 56; 79; 101 | 4.78 | 43; 45; 96 | 6.00 |
| 3; 61; 82 | 3.93 | 23; 84; 119 | 5.06 | 75; 130; 141 | 3.07 | 1; 81; 97 | 5.82 | 26; 49; 101 | 4.78 | 7; 33; 43 | 4.86 |
| 43; 68; 127 | 3.93 | 72; 85; 98 | 5.06 | 72; 112; 135 | 3.07 | 56; 81; 97 | 5.82 | 47; 78; 101 | 4.78 | 43; 70; 91 | 4.86 |
| 3; 29; 119 | 3.93 | 23; 97; 150 | 5.06 | 126; 129; 145 | 3.07 | 14; 28; 89 | 5.82 | 19; 56; 101 | 4.78 | 43; 72; 108 | 4.86 |
| 12; 43; 146 | 3.93 | 18; 84; 88 | 4.07 | 40; 99; 130 | 3.07 | 1; 81; 93 | 4.80 | 19; 24; 39 | 4.78 | 43; 70; 108 | 4.86 |
| 43; 44; 131 | 3.93 | 24; 77; 98 | 4.07 | 40; 101; 130 | 3.07 | 9; 79; 92 | 4.80 | 56; 93; 101 | 4.78 | 43; 72; 91 | 4.86 |
| 41; 42; 43 | 3.93 | 56; 93; 118 | 4.07 | 60; 131; 133 | 3.07 | 4; 26; 97 | 4.80 | 22; 29; 99 | 4.78 | 7; 41; 43 | 4.86 |
| 43; 68; 100 | 3.93 | 29; 68; 98 | 4.07 | 3; 130; 145 | 3.07 | 1; 81; 96 | 4.80 | 22; 39; 102 | 4.78 | 43; 71; 151 | 4.86 |
| 1; 41; 43 | 3.93 | 14; 76; 119 | 4.07 | 75; 130; 145 | 3.07 | 1; 81; 98 | 4.80 | 8; 24; 102 | 4.78 | 43; 69; 91 | 4.86 |
| 23; 41; 43 | 3.93 | 7; 85; 118 | 4.07 | 75; 130; 144 | 3.07 | 4; 26; 81 | 4.80 | 47; 82; 101 | 4.78 | 1; 41; 43 | 4.86 |
| 43; 63; 138 | 3.93 | 73; 98; 135 | 4.07 | 40; 107; 130 | 3.07 | 1; 82; 98 | 4.80 | 15; 18; 100 | 4.78 | 1; 42; 43 | 4.86 |
| 43; 52; 151 | 3.93 | 98; 118; 149 | 4.07 | 89; 131; 142 | 3.07 | 68; 92; 94 | 4.80 | 22; 24; 102 | 4.78 | 1; 43; 54 | 4.86 |
| 36; 43; 146 | 3.93 | 27; 84; 98 | 4.07 | 70; 130; 141 | 3.07 | 4; 89; 97 | 4.80 | 37; 89; 99 | 4.78 | 3; 43; 61 | 4.86 |
| 43; 56; 109 | 3.93 | 24; 76; 98 | 4.07 | 60; 131; 142 | 3.07 | 37; 77; 98 | 4.80 | 19; 26; 83 | 4.78 | 1; 43; 70 | 4.86 |
| 43; 81; 107 | 3.93 | 24; 76; 102 | 4.07 | 19; 40; 130 | 3.07 | 6; 97; 107 | 4.80 | 2; 24; 111 | 4.78 | 1; 43; 68 | 4.86 |
| 3; 70; 84 | 3.93 | 23; 98; 150 | 4.07 | 5; 130; 145 | 3.07 | 1; 83; 93 | 4.80 | 16; 64; 101 | 4.78 | 1; 43; 67 | 4.86 |
| 3; 29; 84 | 3.93 | 26; 73; 97 | 4.07 | 114; 130; 145 | 3.07 | 1; 76; 92 | 4.80 | 56; 86; 89 | 4.78 | 1; 43; 63 | 4.86 |
| 3; 61; 126 | 3.93 | 29; 74; 98 | 4.07 | 40; 96; 130 | 3.07 | 6; 75; 92 | 4.80 | 30; 86; 101 | 4.78 | 1; 43; 62 | 4.86 |
| 3; 61; 119 | 3.93 | 24; 102; 129 | 4.07 | 68; 81; 129 | 3.07 | 56; 79; 94 | 4.80 | 14; 26; 89 | 4.78 | 7; 30; 43 | 4.86 |
| 7; 43; 106 | 3.93 | 29; 93; 118 | 4.07 | 38; 129; 149 | 3.07 | 4; 94; 95 | 4.80 | 16; 41; 101 | 4.78 | 1; 43; 45 | 4.86 |
| 44; 84; 107 | 3.93 | 23; 85; 102 | 4.07 | 14; 129; 145 | 3.07 | 56; 92; 94 | 4.80 | 22; 89; 99 | 4.78 | 20; 42; 44 | 4.86 |
| 43; 73; 106 | 3.93 | 23; 102; 150 | 4.07 | 38; 129; 145 | 3.07 | 85; 94; 98 | 4.80 | 56; 86; 101 | 4.78 | 7; 25; 43 | 4.86 |
| 18; 43; 52 | 3.93 | 67; 102; 150 | 4.07 | 25; 114; 129 | 3.07 | 92; 94; 98 | 4.80 | 13; 28; 101 | 3.97 | 43; 73; 91 | 4.86 |
| 20; 43; 52 | 3.93 | 98; 119; 135 | 4.07 | 114; 130; 150 | 3.07 | 56; 81; 92 | 4.80 | 2; 26; 40 | 3.97 | 3; 43; 45 | 4.86 |
| 43; 72; 106 | 3.93 | 40; 97; 118 | 4.07 | 114; 130; 151 | 3.07 | 56; 81; 94 | 4.80 | 17; 100; 101 | 3.97 | 30; 38; 43 | 4.86 |
| 44; 51; 107 | 3.93 | 54; 102; 133 | 4.07 | 40; 68; 130 | 3.07 | 1; 78; 79 | 4.80 | 15; 30; 101 | 3.97 | 43; 54; 61 | 4.86 |
| 41; 43; 135 | 3.93 | 54; 102; 132 | 4.07 | 82; 130; 144 | 3.07 | 1; 78; 81 | 4.80 | 24; 34; 89 | 3.97 | 43; 73; 108 | 4.86 |
| 41; 43; 142 | 3.93 | 85; 88; 98 | 4.07 | 70; 130; 145 | 3.07 | 1; 78; 83 | 4.80 | 23; 68; 101 | 3.97 | 30; 43; 75 | 4.86 |
| 3; 61; 79 | 3.93 | 29; 79; 97 | 4.07 | 18; 67; 130 | 3.07 | 79; 94; 98 | 4.80 | 8; 16; 98 | 3.97 | 30; 43; 79 | 4.86 |
| 41; 43; 145 | 3.93 | 24; 80; 98 | 4.07 | 70; 130; 144 | 3.07 | 1; 78; 98 | 4.80 | 39; 89; 102 | 3.97 | 7; 43; 108 | 4.86 |
| 43; 83; 106 | 3.93 | 37; 98; 118 | 4.07 | 7; 131; 149 | 3.07 | 68; 88; 94 | 4.80 | 22; 34; 89 | 3.97 | 30; 43; 82 | 4.86 |
| 41; 43; 146 | 3.93 | 29; 44; 98 | 4.07 | 130; 145; 149 | 3.07 | 1; 56; 92 | 4.80 | 29; 56; 101 | 3.97 | 30; 43; 83 | 4.86 |
| 41; 43; 147 | 3.93 | 23; 88; 119 | 4.07 | 56; 70; 114 | 3.07 | 1; 17; 92 | 4.80 | 15; 99; 100 | 3.97 | 30; 43; 84 | 4.86 |
| 41; 43; 148 | 3.93 | 27; 85; 98 | 4.07 | 89; 131; 145 | 3.07 | 70; 94; 98 | 4.80 | 20; 22; 99 | 3.97 | 20; 43; 91 | 4.86 |
| 43; 52; 135 | 3.93 | 67; 88; 102 | 4.07 | 25; 40; 130 | 3.07 | 27; 97; 142 | 4.80 | 8; 16; 101 | 3.97 | 30; 43; 85 | 4.86 |
| 43; 52; 134 | 3.93 | 85; 86; 118 | 4.07 | 11; 129; 135 | 3.07 | 54; 81; 94 | 4.80 | 72; 86; 89 | 3.97 | 30; 43; 86 | 4.86 |
| 41; 43; 132 | 3.93 | 74; 85; 98 | 4.07 | 130; 145; 150 | 3.07 | 1; 7; 92 | 4.80 | 26; 39; 81 | 3.97 | 30; 43; 89 | 4.86 |
| 41; 43; 131 | 3.93 | 23; 88; 102 | 4.07 | 89; 131; 135 | 3.07 | 81; 94; 98 | 4.80 | 8; 18; 101 | 3.97 | 30; 43; 90 | 4.86 |
| 41; 43; 129 | 3.93 | 26; 93; 98 | 4.07 | 130; 145; 146 | 3.07 | 56; 84; 94 | 4.80 | 22; 30; 101 | 3.97 | 20; 43; 108 | 4.86 |
| 9; 43; 100 | 3.93 | 26; 93; 97 | 4.07 | 37; 129; 145 | 3.07 | 1; 77; 98 | 4.80 | 6; 22; 99 | 3.97 | 20; 43; 133 | 4.86 |
| 43; 52; 132 | 3.93 | 9; 24; 98 | 4.07 | 96; 129; 135 | 3.07 | 6; 13; 126 | 4.80 | 23; 72; 101 | 3.97 | 20; 43; 151 | 4.86 |
| 43; 83; 127 | 3.93 | 29; 37; 118 | 4.07 | 62; 112; 135 | 3.07 | 6; 28; 94 | 4.80 | 28; 47; 101 | 3.97 | 43; 74; 108 | 4.86 |
| 41; 43; 126 | 3.93 | 29; 70; 98 | 4.07 | 130; 144; 148 | 3.07 | 28; 94; 98 | 4.80 | 2; 40; 99 | 3.97 | 30; 43; 120 | 4.86 |
| 32; 43; 132 | 3.93 | 23; 97; 107 | 4.07 | 37; 130; 145 | 3.07 | 2; 105; 128 | 4.80 | 26; 47; 79 | 3.97 | 30; 43; 78 | 4.86 |
| 43; 52; 131 | 3.93 | 37; 85; 118 | 4.07 | 64; 114; 129 | 3.07 | 8; 84; 92 | 4.80 | 22; 26; 102 | 3.97 | 30; 43; 73 | 4.86 |
| 37; 43; 52 | 3.93 | 23; 97; 118 | 4.07 | 96; 129; 133 | 3.07 | 68; 79; 94 | 4.80 | 22; 75; 99 | 3.97 | 20; 43; 45 | 4.86 |
| 41; 43; 149 | 3.93 | 27; 64; 98 | 4.07 | 75; 129; 149 | 3.07 | 68; 79; 92 | 4.80 | 15; 98; 99 | 3.97 | 30; 43; 72 | 4.86 |
| 41; 43; 150 | 3.93 | 54; 74; 102 | 4.07 | 87; 114; 130 | 3.07 | 1; 96; 148 | 4.80 | 21; 22; 101 | 3.97 | 1; 8; 43 | 4.86 |
| 3; 29; 150 | 3.93 | 24; 29; 98 | 4.07 | 79; 130; 144 | 3.07 | 1; 38; 98 | 4.80 | 24; 51; 105 | 3.97 | 1; 43; 75 | 4.86 |
| 3; 70; 86 | 3.93 | 25; 99; 102 | 4.07 | 62; 108; 130 | 3.07 | 79; 89; 94 | 4.80 | 23; 74; 89 | 3.97 | 7; 43; 45 | 4.86 |
| 3; 61; 78 | 3.93 | 27; 73; 98 | 4.07 | 84; 129; 143 | 3.07 | 1; 98; 138 | 4.80 | 22; 38; 99 | 3.97 | 20; 43; 53 | 4.86 |
| 33; 43; 109 | 3.93 | 24; 29; 102 | 4.07 | 7; 130; 144 | 3.07 | 1; 98; 147 | 4.80 | 98; 99; 110 | 3.97 | 20; 43; 54 | 4.86 |
| 15; 43; 81 | 3.93 | 24; 56; 98 | 4.07 | 60; 130; 135 | 3.07 | 1; 98; 148 | 4.80 | 33; 39; 102 | 3.97 | 7; 43; 54 | 4.86 |
| 43; 52; 148 | 3.93 | 23; 97; 119 | 4.07 | 129; 131; 135 | 3.07 | 8; 85; 92 | 4.80 | 22; 73; 99 | 3.97 | 43; 74; 91 | 4.86 |
| 27; 43; 142 | 3.93 | 28; 56; 98 | 4.07 | 19; 130; 131 | 3.07 | 15; 82; 89 | 4.80 | 22; 73; 98 | 3.97 | 43; 74; 96 | 4.86 |
| 43; 44; 110 | 3.93 | 23; 98; 118 | 4.07 | 78; 130; 144 | 3.07 | 1; 93; 98 | 4.80 | 4; 24; 102 | 3.97 | 33; 43; 150 | 4.86 |
| 43; 44; 109 | 3.93 | 28; 98; 102 | 4.07 | 56; 114; 135 | 3.07 | 56; 89; 94 | 4.80 | 33; 86; 89 | 3.97 | 30; 43; 62 | 4.86 |
| 43; 52; 146 | 3.93 | 23; 98; 126 | 4.07 | 60; 67; 130 | 3.07 | 1; 93; 96 | 4.80 | 14; 84; 89 | 3.97 | 30; 43; 63 | 4.86 |
| 43; 44; 106 | 3.93 | 29; 97; 126 | 4.07 | 20; 114; 130 | 3.07 | 4; 81; 92 | 4.80 | 13; 24; 94 | 3.97 | 30; 43; 66 | 4.86 |
| 3; 29; 147 | 3.93 | 23; 98; 134 | 4.07 | 89; 131; 149 | 3.07 | 4; 92; 94 | 4.80 | 56; 78; 101 | 3.97 | 7; 43; 91 | 4.86 |
| 33; 43; 52 | 3.93 | 24; 107; 119 | 4.07 | 96; 112; 135 | 3.07 | 73; 86; 89 | 4.80 | 2; 40; 110 | 3.97 | 30; 43; 71 | 4.86 |
| 3; 41; 43 | 3.93 | 29; 75; 98 | 4.07 | 48; 130; 144 | 3.07 | 1; 89; 98 | 4.80 | 22; 26; 99 | 3.97 | 7; 43; 96 | 4.86 |
| 43; 52; 142 | 3.93 | 26; 76; 97 | 4.07 | 73; 130; 145 | 3.07 | 1; 98; 149 | 4.80 | 8; 22; 102 | 3.97 | 1; 43; 74 | 4.86 |
| 3; 61; 70 | 3.93 | 23; 98; 102 | 4.07 | 97; 131; 135 | 3.07 | 79; 83; 94 | 4.80 | 16; 26; 39 | 3.97 | 1; 43; 76 | 4.86 |
| 43; 52; 140 | 3.93 | 29; 97; 108 | 4.07 | 21; 114; 130 | 3.07 | 1; 75; 98 | 4.80 | 15; 87; 99 | 3.97 | 43; 45; 147 | 4.86 |
| 3; 61; 68 | 3.93 | 23; 97; 126 | 4.07 | 6; 130; 145 | 3.07 | 12; 54; 100 | 4.80 | 15; 26; 78 | 3.97 | 43; 63; 117 | 4.86 |
| 1; 61; 132 | 3.93 | 23; 97; 129 | 4.07 | 68; 89; 129 | 3.07 | 1; 68; 97 | 4.80 | 39; 71; 98 | 3.97 | 43; 63; 118 | 4.86 |
| 43; 96; 106 | 3.93 | 23; 25; 102 | 4.07 | 40; 72; 130 | 3.07 | 1; 68; 96 | 4.80 | 8; 22; 99 | 3.97 | 6; 43; 61 | 4.86 |
| 44; 76; 107 | 3.93 | 27; 56; 98 | 4.07 | 73; 129; 149 | 3.07 | 6; 15; 98 | 4.80 | 22; 79; 99 | 3.97 | 6; 43; 62 | 4.86 |
| 43; 78; 106 | 3.93 | 23; 97; 147 | 4.07 | 40; 81; 130 | 3.07 | 6; 16; 56 | 3.86 | 22; 89; 101 | 3.97 | 6; 43; 70 | 4.86 |
| 43; 94; 138 | 3.93 | 23; 97; 149 | 4.07 | 135; 143; 149 | 3.07 | 15; 82; 92 | 3.86 | 56; 73; 89 | 3.97 | 6; 43; 72 | 4.86 |
| 43; 75; 109 | 3.93 | 23; 93; 118 | 4.07 | 110; 132; 148 | 3.07 | 6; 13; 79 | 3.86 | 2; 18; 111 | 3.97 | 6; 43; 73 | 4.86 |
| 36; 41; 43 | 3.93 | 24; 54; 98 | 4.07 | 8; 129; 135 | 3.07 | 4; 13; 90 | 3.86 | 18; 24; 26 | 3.97 | 6; 43; 74 | 4.86 |
| 16; 37; 43 | 3.93 | 26; 84; 97 | 4.07 | 72; 129; 149 | 3.07 | 1; 13; 81 | 3.86 | 16; 26; 56 | 3.97 | 6; 43; 82 | 4.86 |
| 39; 43; 107 | 3.93 | 29; 76; 119 | 4.07 | 107; 130; 145 | 3.07 | 4; 12; 92 | 3.86 | 7; 30; 98 | 3.97 | 6; 43; 83 | 4.86 |
| 43; 134; 146 | 3.93 | 76; 102; 142 | 4.07 | 96; 114; 135 | 3.07 | 6; 15; 81 | 3.86 | 15; 39; 99 | 3.97 | 6; 43; 84 | 4.86 |
| 29; 39; 43 | 3.93 | 29; 76; 102 | 4.07 | 114; 130; 141 | 3.07 | 1; 15; 37 | 3.86 | 55; 74; 89 | 3.97 | 1; 5; 43 | 4.86 |
| 34; 43; 60 | 3.93 | 26; 83; 97 | 4.07 | 40; 73; 130 | 3.07 | 6; 12; 100 | 3.86 | 22; 89; 102 | 3.97 | 43; 45; 150 | 4.86 |
| 39; 43; 147 | 3.93 | 24; 69; 98 | 4.07 | 109; 130; 143 | 3.07 | 6; 16; 72 | 3.86 | 16; 89; 101 | 3.97 | 1; 43; 83 | 4.86 |
| 43; 46; 124 | 3.93 | 29; 76; 98 | 4.07 | 54; 86; 114 | 3.07 | 6; 16; 70 | 3.86 | 16; 18; 101 | 3.97 | 43; 61; 111 | 4.86 |
| 43; 103; 120 | 3.93 | 26; 85; 97 | 4.07 | 4; 75; 130 | 3.07 | 6; 13; 15 | 3.86 | 22; 74; 99 | 3.97 | 43; 61; 118 | 4.86 |
| 43; 55; 101 | 3.93 | 24; 102; 150 | 4.07 | 3; 8; 130 | 3.07 | 4; 13; 78 | 3.86 | 15; 18; 99 | 3.97 | 43; 61; 133 | 4.86 |
| 30; 41; 43 | 3.93 | 33; 54; 102 | 4.07 | 25; 130; 141 | 3.07 | 4; 13; 81 | 3.86 | 39; 63; 102 | 3.97 | 6; 37; 43 | 4.86 |
| 43; 46; 140 | 3.93 | 37; 84; 118 | 4.07 | 54; 85; 114 | 3.07 | 8; 20; 97 | 3.86 | 17; 26; 63 | 3.97 | 6; 41; 43 | 4.86 |
| 43; 46; 141 | 3.93 | 25; 67; 102 | 4.07 | 69; 112; 135 | 3.07 | 6; 15; 97 | 3.86 | 8; 22; 120 | 3.97 | 43; 62; 91 | 4.86 |
| 39; 43; 149 | 3.93 | 23; 93; 97 | 4.07 | 109; 114; 130 | 3.07 | 79; 92; 98 | 3.86 | 14; 89; 93 | 3.97 | 43; 62; 108 | 4.86 |
| 43; 133; 146 | 3.93 | 64; 94; 118 | 4.07 | 33; 130; 145 | 3.07 | 17; 81; 89 | 3.86 | 18; 24; 102 | 3.97 | 3; 43; 150 | 4.86 |
| 43; 65; 103 | 3.93 | 25; 98; 126 | 4.07 | 40; 69; 130 | 3.07 | 6; 14; 97 | 3.86 | 22; 33; 99 | 3.97 | 43; 63; 96 | 4.86 |

| Lymphatic | | | | MUC13_DMBT1 | | | | | | | |
|---|---|---|---|---|---|---|---|---|---|---|---|
| endothelial cells | | Lymphoid cells | | positive cells | | Megakaryocytes | | Mesangial cells | | Mesothelial cells | |
| Clusters | Score | Clusters | Score | Clusters | Score | Clusters | Score | Clusters | Score | Clusters | Score |
| 100; 106; 130 | 5.73 | 40; 41; 108 | 5.34 | 31; 82; 125 | 5.06 | 36; 57; 130 | 3.76 | 51; 88; 112 | 11.14 | 22; 121; 149 | 3.93 |
| 108; 120; 149 | 5.73 | 10; 40; 145 | 5.34 | 43; 63; 96 | 5.06 | 8; 64; 100 | 3.76 | 27; 111; 122 | 11.14 | 40; 105; 141 | 3.93 |
| 44; 121; 129 | 5.73 | 41; 91; 135 | 5.34 | 31; 72; 125 | 5.06 | 56; 86; 149 | 3.76 | 51; 111; 120 | 11.14 | 29; 89; 138 | 2.81 |
| 72; 77; 120 | 5.73 | 6; 91; 135 | 5.34 | 41; 43; 63 | 5.06 | 46; 48; 64 | 3.76 | 34; 95; 111 | 11.14 | 39; 70; 103 | 2.81 |
| 100; 106; 149 | 5.73 | 17; 40; 107 | 4.31 | 31; 83; 125 | 5.06 | 36; 56; 130 | 3.76 | 34; 51; 111 | 9.88 | 40; 41; 105 | 2.81 |
| 26; 129; 149 | 5.73 | 3; 34; 40 | 4.31 | 7; 43; 125 | 4.07 | 36; 49; 130 | 3.76 | 44; 111; 120 | 9.88 | 40; 88; 121 | 2.81 |
| 72; 108; 149 | 5.73 | 40; 71; 133 | 4.31 | 43; 64; 125 | 4.07 | 8; 57; 100 | 3.76 | 51; 112; 120 | 9.88 | 5; 105; 115 | 2.81 |
| 100; 130; 145 | 5.73 | 6; 55; 135 | 4.31 | 17; 43; 83 | 4.07 | 48; 64; 100 | 3.76 | 51; 71; 112 | 9.88 | 45; 78; 143 | 2.81 |
| 100; 130; 140 | 5.73 | 17; 40; 41 | 4.31 | 43; 44; 125 | 4.07 | 48; 55; 100 | 3.76 | 94; 111; 122 | 9.88 | 39; 68; 103 | 2.81 |
| 9; 77; 129 | 5.73 | 40; 70; 133 | 4.31 | 63; 125; 150 | 4.07 | 48; 57; 119 | 2.68 | 51; 82; 111 | 9.88 | 62; 96; 102 | 2.81 |
| 44; 120; 129 | 5.73 | 40; 41; 129 | 4.31 | 43; 73; 81 | 4.07 | 1; 48; 64 | 2.68 | 55; 111; 122 | 9.88 | 67; 105; 115 | 2.81 |
| 120; 129; 133 | 5.73 | 6; 45; 135 | 4.31 | 17; 43; 82 | 4.07 | 48; 57; 130 | 2.68 | 54; 111; 122 | 9.88 | 40; 86; 121 | 2.81 |
| 77; 120; 126 | 5.73 | 17; 40; 142 | 4.31 | 17; 43; 81 | 4.07 | 51; 75; 147 | 2.68 | 35; 111; 122 | 9.88 | 40; 85; 121 | 2.81 |
| 44; 100; 121 | 4.35 | 40; 70; 91 | 4.31 | 38; 84; 125 | 4.07 | 5; 51; 64 | 2.68 | 19; 111; 122 | 9.88 | 5; 40; 121 | 2.81 |
| 100; 129; 151 | 4.35 | 41; 94; 135 | 4.31 | 37; 44; 125 | 4.07 | 29; 62; 100 | 2.68 | 29; 34; 111 | 9.88 | 97; 143; 150 | 2.81 |
| 71; 100; 129 | 4.35 | 41; 98; 135 | 4.31 | 43; 77; 125 | 4.07 | 48; 57; 138 | 2.68 | 33; 111; 122 | 9.88 | 40; 84; 121 | 2.81 |
| 46; 120; 138 | 4.35 | 6; 69; 135 | 4.31 | 31; 70; 125 | 4.07 | 51; 75; 150 | 2.68 | 34; 56; 111 | 9.88 | 39; 64; 115 | 2.81 |
| 55; 108; 120 | 4.35 | 41; 107; 132 | 4.31 | 31; 44; 125 | 4.07 | 5; 48; 71 | 2.68 | 25; 111; 122 | 9.88 | 40; 83; 121 | 2.81 |
| 71; 100; 130 | 4.35 | 40; 69; 133 | 4.31 | 5; 41; 43 | 4.07 | 51; 75; 86 | 2.68 | 16; 112; 122 | 9.88 | 41; 104; 142 | 2.81 |
| 102; 145; 146 | 4.35 | 39; 69; 133 | 4.31 | 31; 81; 125 | 4.07 | 10; 55; 100 | 2.68 | 67; 111; 122 | 9.88 | 22; 39; 115 | 2.81 |
| 100; 145; 146 | 4.35 | 37; 40; 133 | 4.31 | 43; 125; 135 | 4.07 | 48; 57; 149 | 2.68 | 56; 111; 120 | 9.88 | 41; 105; 141 | 2.81 |
| 23; 124; 129 | 4.35 | 40; 75; 133 | 4.31 | 43; 72; 83 | 4.07 | 13; 28; 36 | 2.68 | 57; 111; 122 | 9.88 | 41; 105; 145 | 2.81 |
| 100; 106; 120 | 4.35 | 40; 79; 133 | 4.31 | 43; 72; 81 | 4.07 | 48; 57; 148 | 2.68 | 29; 111; 122 | 9.88 | 12; 40; 121 | 2.81 |
| 55; 77; 121 | 4.35 | 9; 40; 148 | 4.31 | 43; 79; 125 | 4.07 | 48; 57; 147 | 2.68 | 51; 93; 112 | 9.88 | 40; 100; 141 | 2.81 |
| 23; 120; 133 | 4.35 | 40; 79; 91 | 4.31 | 63; 82; 125 | 4.07 | 46; 94; 130 | 2.68 | 61; 111; 122 | 9.88 | 10; 40; 121 | 2.81 |
| 61; 100; 130 | 4.35 | 6; 37; 135 | 4.31 | 38; 43; 125 | 4.07 | 50; 130; 138 | 2.68 | 62; 111; 120 | 9.88 | 55; 103; 119 | 2.81 |
| 1; 77; 121 | 4.35 | 40; 78; 91 | 4.31 | 43; 72; 125 | 4.07 | 18; 47; 138 | 2.68 | 46; 74; 111 | 8.68 | 5; 116; 123 | 2.81 |
| 100; 108; 121 | 4.35 | 39; 40; 125 | 4.31 | 43; 64; 96 | 4.07 | 60; 65; 123 | 2.68 | 28; 112; 127 | 8.68 | 22; 113; 149 | 2.81 |
| 23; 108; 120 | 4.35 | 40; 76; 125 | 4.31 | 7; 43; 96 | 4.07 | 38; 52; 65 | 2.68 | 21; 42; 112 | 8.68 | 22; 113; 146 | 2.81 |
| 102; 145; 151 | 4.35 | 6; 39; 133 | 4.31 | 31; 55; 125 | 4.07 | 36; 56; 138 | 2.68 | 69; 111; 122 | 8.68 | 22; 113; 145 | 2.81 |
| 77; 120; 124 | 4.35 | 39; 68; 133 | 4.31 | 41; 43; 71 | 4.07 | 60; 64; 136 | 2.68 | 102; 111; 122 | 8.68 | 39; 73; 103 | 2.81 |
| 7; 77; 120 | 4.35 | 6; 40; 41 | 4.31 | 44; 125; 150 | 4.07 | 48; 57; 100 | 2.68 | 23; 56; 112 | 8.68 | 6; 78; 147 | 2.81 |
| 23; 120; 126 | 4.35 | 40; 74; 133 | 4.31 | 29; 83; 125 | 4.07 | 50; 130; 147 | 2.68 | 23; 86; 112 | 8.68 | 64; 103; 119 | 2.81 |
| 44; 108; 120 | 4.35 | 6; 40; 133 | 4.31 | 84; 125; 150 | 4.07 | 38; 51; 147 | 2.68 | 26; 111; 122 | 8.68 | 40; 93; 121 | 2.81 |
| 72; 100; 121 | 4.35 | 6; 40; 145 | 4.31 | 41; 43; 68 | 4.07 | 38; 51; 150 | 2.68 | 44; 71; 111 | 8.68 | 67; 116; 123 | 2.81 |
| 23; 72; 120 | 4.35 | 40; 73; 133 | 4.31 | 38; 44; 125 | 4.07 | 50; 130; 148 | 2.68 | 51; 75; 111 | 8.68 | 64; 105; 141 | 2.81 |
| 100; 120; 128 | 4.35 | 40; 73; 91 | 4.31 | 41; 43; 79 | 4.07 | 57; 71; 136 | 2.68 | 34; 85; 111 | 8.68 | 61; 92; 143 | 2.81 |
| 45; 102; 145 | 4.35 | 6; 75; 135 | 4.31 | 31; 63; 125 | 4.07 | 48; 62; 100 | 2.68 | 51; 79; 112 | 8.68 | 40; 101; 141 | 2.81 |
| 121; 129; 151 | 4.35 | 20; 40; 125 | 4.31 | 41; 43; 94 | 4.07 | 33; 61; 86 | 2.68 | 71; 111; 126 | 8.68 | 40; 71; 138 | 2.81 |
| 100; 145; 149 | 4.35 | 39; 73; 133 | 4.31 | 41; 43; 92 | 4.07 | 48; 62; 86 | 2.68 | 38; 51; 112 | 8.68 | 40; 71; 117 | 2.81 |
| 81; 100; 121 | 4.35 | 8; 133; 135 | 4.31 | 41; 43; 98 | 4.07 | 48; 62; 85 | 2.68 | 71; 111; 122 | 8.68 | 40; 71; 114 | 2.81 |
| 23; 129; 146 | 4.35 | 39; 74; 133 | 4.31 | 43; 87; 151 | 4.07 | 48; 62; 84 | 2.68 | 38; 51; 111 | 8.68 | 19; 105; 115 | 2.81 |
| 100; 145; 150 | 4.35 | 39; 76; 133 | 4.31 | 43; 50; 62 | 4.07 | 48; 62; 83 | 2.68 | 21; 71; 112 | 8.68 | 59; 101; 117 | 2.81 |
| 100; 129; 142 | 4.35 | 40; 57; 125 | 4.31 | 7; 41; 43 | 4.07 | 48; 62; 82 | 2.68 | 6; 111; 122 | 8.68 | 45; 105; 115 | 2.81 |
| 106; 122; 130 | 4.35 | 39; 84; 133 | 4.31 | 63; 84; 133 | 4.07 | 46; 79; 130 | 2.68 | 6; 111; 127 | 8.68 | 8; 95; 125 | 2.81 |
| 44; 100; 129 | 4.35 | 44; 45; 133 | 4.31 | 43; 66; 125 | 4.07 | 48; 62; 79 | 2.68 | 47; 65; 111 | 8.68 | 59; 102; 114 | 2.81 |
| 77; 120; 129 | 4.35 | 54; 68; 135 | 4.31 | 41; 43; 84 | 4.07 | 48; 62; 75 | 2.68 | 34; 84; 111 | 8.68 | 117; 120; 142 | 2.81 |
| 61; 100; 118 | 4.35 | 40; 55; 125 | 4.31 | 26; 41; 43 | 4.07 | 48; 62; 73 | 2.68 | 16; 29; 112 | 8.68 | 40; 70; 121 | 2.81 |
| 92; 100; 129 | 4.35 | 40; 55; 108 | 4.31 | 31; 84; 125 | 4.07 | 9; 86; 150 | 2.68 | 38; 111; 122 | 8.68 | 76; 105; 141 | 2.81 |
| 20; 120; 129 | 4.35 | 44; 129; 133 | 4.31 | 43; 73; 125 | 4.07 | 46; 80; 130 | 2.68 | 51; 102; 111 | 8.68 | 71; 88; 146 | 2.81 |
| 106; 130; 145 | 4.35 | 3; 133; 135 | 4.31 | 43; 55; 81 | 4.07 | 48; 62; 71 | 2.68 | 21; 44; 112 | 8.68 | 56; 105; 115 | 2.81 |
| 106; 130; 146 | 4.35 | 40; 54; 125 | 4.31 | 37; 43; 71 | 4.07 | 46; 77; 130 | 2.68 | 55; 95; 111 | 8.68 | 9; 40; 121 | 2.81 |
| 122; 130; 145 | 4.35 | 45; 129; 133 | 4.31 | 38; 82; 125 | 4.07 | 46; 81; 130 | 2.68 | 85; 111; 122 | 8.68 | 9; 105; 115 | 2.81 |
| 23; 120; 147 | 4.35 | 45; 129; 135 | 4.31 | 43; 93; 125 | 4.07 | 48; 62; 120 | 2.68 | 5; 112; 127 | 8.68 | 5; 39; 103 | 2.81 |
| 100; 129; 145 | 4.35 | 68; 91; 135 | 4.31 | 37; 43; 62 | 4.07 | 5; 56; 75 | 2.68 | 78; 111; 122 | 8.68 | 40; 81; 105 | 2.81 |
| 122; 130; 141 | 4.35 | 3; 41; 135 | 4.31 | 37; 43; 64 | 4.07 | 9; 55; 75 | 2.68 | 51; 77; 112 | 8.68 | 40; 43; 145 | 2.81 |
| 34; 118; 130 | 4.35 | 40; 63; 91 | 4.31 | 44; 73; 125 | 4.07 | 13; 19; 64 | 2.68 | 96; 111; 122 | 8.68 | 40; 79; 121 | 2.81 |
| 44; 72; 120 | 4.35 | 25; 40; 41 | 4.31 | 31; 64; 125 | 4.07 | 9; 55; 73 | 2.68 | 44; 51; 112 | 8.68 | 22; 40; 121 | 2.81 |
| 23; 120; 148 | 4.35 | 40; 63; 133 | 4.31 | 41; 43; 45 | 4.07 | 49; 120; 147 | 2.68 | 28; 112; 120 | 8.68 | 39; 62; 115 | 2.81 |
| 66; 120; 129 | 4.35 | 41; 126; 135 | 4.31 | 43; 81; 143 | 4.07 | 48; 62; 145 | 2.68 | 29; 30; 112 | 8.68 | 41; 119; 143 | 2.81 |
| 23; 129; 141 | 4.35 | 54; 135; 147 | 4.31 | 31; 79; 125 | 4.07 | 8; 23; 64 | 2.68 | 34; 83; 111 | 8.68 | 40; 45; 121 | 2.81 |
| 5; 102; 145 | 4.35 | 41; 129; 135 | 4.31 | 43; 62; 138 | 4.07 | 19; 60; 130 | 2.68 | 85; 111; 128 | 8.68 | 22; 41; 121 | 2.81 |
| 118; 129; 145 | 4.35 | 6; 79; 135 | 4.31 | 10; 41; 43 | 4.07 | 37; 48; 147 | 2.68 | 9; 111; 120 | 8.68 | 39; 59; 138 | 2.81 |
| 85; 108; 120 | 4.35 | 40; 68; 151 | 4.31 | 63; 64; 125 | 4.07 | 48; 62; 126 | 2.68 | 111; 116; 122 | 8.68 | 5; 92; 143 | 2.81 |
| 44; 120; 149 | 4.35 | 6; 83; 135 | 4.31 | 43; 63; 118 | 4.07 | 33; 38; 65 | 2.68 | 20; 111; 122 | 8.68 | 82; 105; 115 | 2.81 |
| 17; 80; 121 | 4.35 | 6; 84; 135 | 4.31 | 6; 43; 125 | 4.07 | 46; 82; 130 | 2.68 | 97; 111; 122 | 8.68 | 41; 143; 147 | 2.81 |
| 35; 80; 120 | 4.35 | 40; 68; 133 | 4.31 | 43; 62; 66 | 4.07 | 36; 55; 130 | 2.68 | 29; 88; 112 | 8.68 | 13; 103; 141 | 2.81 |
| 124; 130; 140 | 4.35 | 6; 88; 135 | 4.31 | 10; 43; 77 | 4.07 | 38; 51; 71 | 2.68 | 51; 98; 112 | 8.68 | 88; 121; 146 | 2.81 |
| 44; 108; 149 | 4.35 | 40; 68; 108 | 4.31 | 43; 63; 125 | 4.07 | 46; 85; 130 | 2.68 | 19; 112; 122 | 8.68 | 40; 120; 138 | 2.81 |
| 44; 72; 149 | 4.35 | 70; 91; 135 | 4.31 | 10; 43; 78 | 4.07 | 38; 51; 73 | 2.68 | 51; 96; 111 | 8.68 | 40; 119; 121 | 2.81 |
| 44; 120; 147 | 4.35 | 6; 90; 135 | 4.31 | 10; 43; 81 | 4.07 | 38; 51; 75 | 2.68 | 52; 112; 120 | 8.68 | 105; 115; 147 | 2.81 |
| 55; 77; 129 | 4.35 | 31; 40; 96 | 4.31 | 43; 62; 64 | 4.07 | 46; 86; 130 | 2.68 | 47; 95; 111 | 8.68 | 39; 92; 103 | 2.81 |
| 107; 108; 120 | 4.35 | 40; 67; 125 | 4.31 | 43; 62; 70 | 4.07 | 38; 51; 82 | 2.68 | 23; 62; 112 | 8.68 | 6; 92; 143 | 2.81 |
| 100; 120; 150 | 4.35 | 20; 41; 135 | 4.31 | 43; 62; 132 | 4.07 | 38; 51; 83 | 2.68 | 111; 118; 122 | 8.68 | 39; 103; 119 | 2.81 |
| 100; 120; 141 | 4.35 | 4; 40; 107 | 4.31 | 38; 43; 64 | 4.07 | 36; 54; 130 | 2.68 | 34; 65; 111 | 8.68 | 40; 117; 121 | 2.81 |
| 108; 120; 129 | 4.35 | 3; 40; 83 | 4.31 | 43; 62; 73 | 4.07 | 25; 91; 130 | 2.68 | 44; 88; 112 | 8.68 | 40; 117; 120 | 2.81 |
| 100; 130; 142 | 4.35 | 40; 79; 145 | 4.31 | 43; 62; 63 | 4.07 | 60; 62; 136 | 2.68 | 34; 64; 111 | 8.68 | 54; 83; 138 | 2.81 |
| 100; 130; 141 | 4.35 | 41; 60; 91 | 4.31 | 63; 83; 125 | 4.07 | 38; 51; 70 | 2.68 | 35; 65; 112 | 8.68 | 40; 116; 138 | 2.81 |
| 26; 118; 130 | 4.35 | 40; 91; 119 | 4.31 | 44; 84; 133 | 4.07 | 46; 84; 130 | 2.68 | 112; 122; 129 | 8.68 | 40; 116; 121 | 2.81 |
| 108; 120; 133 | 4.35 | 40; 91; 146 | 4.31 | 43; 63; 78 | 4.07 | 5; 55; 56 | 2.68 | 52; 111; 122 | 8.68 | 40; 114; 142 | 2.81 |
| 118; 130; 140 | 4.35 | 10; 40; 129 | 4.31 | 43; 63; 81 | 4.07 | 36; 53; 130 | 2.68 | 34; 68; 111 | 8.68 | 95; 114; 143 | 2.81 |
| 108; 120; 151 | 4.35 | 40; 91; 141 | 4.31 | 43; 81; 125 | 4.07 | 39; 46; 130 | 2.68 | 51; 84; 112 | 8.68 | 63; 105; 115 | 2.81 |
| 72; 108; 120 | 4.35 | 40; 126; 145 | 4.31 | 43; 62; 150 | 4.07 | 46; 83; 130 | 2.68 | 51; 95; 111 | 8.68 | 105; 141; 142 | 2.81 |
| 77; 79; 121 | 4.35 | 10; 40; 119 | 4.31 | 43; 82; 125 | 4.07 | 27; 60; 120 | 2.68 | 28; 71; 111 | 8.68 | 63; 105; 141 | 2.81 |
| 46; 126; 149 | 4.35 | 40; 91; 133 | 4.31 | 38; 43; 71 | 4.07 | 36; 56; 149 | 2.68 | 22; 112; 120 | 8.68 | 29; 40; 121 | 2.81 |
| 46; 124; 146 | 4.35 | 10; 40; 107 | 4.31 | 31; 77; 125 | 4.07 | 44; 47; 65 | 2.68 | 112; 122; 135 | 8.68 | 40; 121; 150 | 2.81 |
| 26; 118; 129 | 4.35 | 40; 91; 120 | 4.31 | 43; 49; 64 | 4.07 | 34; 46; 130 | 2.68 | 111; 127; 135 | 8.68 | 40; 121; 148 | 2.81 |
| 46; 92; 149 | 4.35 | 40; 129; 133 | 4.31 | 43; 62; 83 | 4.07 | 13; 37; 149 | 2.68 | 95; 111; 151 | 8.68 | 40; 121; 145 | 2.81 |
| 26; 118; 149 | 4.35 | 40; 81; 91 | 4.31 | 43; 70; 125 | 4.07 | 13; 37; 150 | 2.68 | 111; 119; 122 | 8.68 | 40; 121; 142 | 2.81 |
| 91; 129; 149 | 4.35 | 40; 91; 109 | 4.31 | 44; 83; 125 | 4.07 | 9; 47; 150 | 2.68 | 10; 108; 112 | 8.68 | 40; 121; 141 | 2.81 |
| 80; 100; 118 | 4.35 | 40; 132; 133 | 4.31 | 43; 64; 73 | 4.07 | 9; 47; 149 | 2.68 | 23; 93; 112 | 8.68 | 40; 121; 138 | 2.81 |
| 23; 101; 120 | 4.35 | 10; 40; 92 | 4.31 | 43; 84; 125 | 4.07 | 9; 47; 148 | 2.68 | 43; 111; 122 | 8.68 | 50; 105; 115 | 2.81 |
| 101; 130; 145 | 4.35 | 40; 90; 132 | 4.31 | 8; 41; 43 | 4.07 | 48; 55; 81 | 2.68 | 44; 95; 111 | 8.68 | 40; 114; 121 | 2.81 |
| 79; 120; 129 | 4.35 | 10; 40; 85 | 4.31 | 38; 43; 62 | 4.07 | 47; 49; 136 | 2.68 | 85; 112; 120 | 8.68 | 40; 110; 113 | 2.81 |
| 34; 120; 129 | 4.35 | 40; 125; 151 | 4.31 | 6; 35; 43 | 4.07 | 9; 47; 147 | 2.68 | 37; 95; 111 | 8.68 | 36; 39; 115 | 2.81 |
| 106; 118; 130 | 4.35 | 40; 125; 129 | 4.31 | 6; 43; 53 | 4.07 | 9; 47; 145 | 2.68 | 23; 54; 112 | 8.68 | 39; 83; 103 | 2.81 |
| 118; 130; 145 | 4.35 | 55; 91; 135 | 4.31 | 6; 43; 45 | 4.07 | 9; 47; 140 | 2.68 | 62; 71; 111 | 8.68 | 41; 61; 121 | 2.81 |
| 70; 100; 121 | 4.35 | 40; 120; 142 | 4.31 | 43; 125; 150 | 4.07 | 9; 47; 138 | 2.68 | 34; 75; 111 | 8.68 | 39; 82; 103 | 2.81 |
| 120; 133; 149 | 4.35 | 40; 107; 132 | 4.31 | 43; 83; 125 | 4.07 | 55; 56; 100 | 2.68 | 34; 74; 111 | 8.68 | 22; 121; 142 | 2.81 |
| 108; 120; 142 | 4.35 | 40; 101; 133 | 4.31 | 43; 62; 81 | 4.07 | 13; 37; 70 | 2.68 | 10; 112; 127 | 8.68 | 39; 81; 103 | 2.81 |

| | | | | Neuroendocrine | | | | PAEP_MECOM | | PDE11A_FAM19A2 | |
|---|---|---|---|---|---|---|---|---|---|---|---|
| Metanephric cells | | Myeloid cells | | cells | | Oligodendrocytes | | positive cells | | positive cells | |
| Clusters | Score | Clusters | Score | Clusters | Score | Clusters | Score | Clusters | Score | Clusters | Score |
| 4; 86; 110 | 14.10 | 30; 41; 60 | 3.30 | 70; 88; 147 | 5.38 | 94; 97; 101 | 5.19 | 35; 62; 133 | 6.00 | 37; 125; 135 | 4.39 |
| 15; 79; 126 | 14.10 | 26; 60; 114 | 3.30 | 29; 88; 147 | 5.38 | 17; 45; 81 | 5.19 | 35; 55; 114 | 6.00 | 25; 97; 135 | 4.39 |
| 4; 15; 37 | 14.10 | 57; 75; 80 | 3.30 | 26; 68; 132 | 4.23 | 89; 95; 98 | 5.19 | 38; 74; 108 | 6.00 | 50; 99; 136 | 4.39 |
| 15; 85; 135 | 14.10 | 50; 57; 125 | 3.30 | 39; 43; 102 | 4.23 | 16; 83; 89 | 5.19 | 72; 74; 108 | 6.00 | 9; 59; 127 | 3.31 |
| 15; 85; 126 | 14.10 | 50; 80; 114 | 3.30 | 28; 118; 131 | 4.23 | 37; 79; 89 | 5.19 | 35; 37; 141 | 6.00 | 127; 133; 135 | 3.31 |
| 24; 102; 112 | 14.10 | 47; 132; 14 | 3.30 | 29; 88; 101 | 4.23 | 17; 101; 107 | 5.19 | 35; 108; 129 | 4.86 | 14; 47; 136 | 3.31 |
| 15; 25; 126 | 14.10 | 57; 73; 80 | 3.30 | 27; 118; 131 | 4.23 | 29; 89; 120 | 5.19 | 31; 35; 141 | 4.86 | 54; 127; 132 | 3.31 |
| 17; 52; 110 | 14.10 | 4; 60; 114 | 3.30 | 27; 101; 131 | 4.23 | 17; 81; 89 | 5.19 | 31; 38; 74 | 4.86 | 72; 97; 135 | 3.31 |
| 15; 29; 85 | 14.10 | 57; 68; 80 | 3.30 | 29; 83; 88 | 4.23 | 82; 92; 98 | 4.08 | 35; 62; 108 | 4.86 | 59; 127; 146 | 3.31 |
| 1; 5; 110 | 14.10 | 4; 50; 114 | 3.30 | 68; 88; 147 | 4.23 | 16; 89; 150 | 4.08 | 35; 38; 149 | 4.86 | 84; 98; 135 | 3.31 |
| 15; 28; 126 | 14.10 | 57; 88; 148 | 3.30 | 26; 68; 119 | 4.23 | 17; 62; 89 | 4.08 | 35; 74; 108 | 4.86 | 25; 125; 135 | 3.31 |
| 28; 102; 111 | 14.10 | 50; 87; 114 | 3.30 | 27; 91; 131 | 4.23 | 17; 79; 89 | 4.08 | 31; 64; 108 | 4.86 | 60; 101; 146 | 3.31 |
| 15; 28; 135 | 14.10 | 41; 57; 81 | 3.30 | 26; 68; 85 | 4.23 | 29; 79; 89 | 4.08 | 38; 53; 148 | 4.86 | 59; 127; 148 | 3.31 |
| 15; 26; 68 | 14.10 | 47; 81; 132 | 3.30 | 46; 100; 114 | 4.23 | 85; 99; 122 | 4.08 | 35; 74; 140 | 4.86 | 43; 59; 127 | 3.31 |
| 15; 86; 126 | 14.10 | 41; 47; 83 | 3.30 | 26; 68; 84 | 4.23 | 29; 99; 122 | 4.08 | 31; 37; 62 | 4.86 | 28; 62; 136 | 3.31 |
| 15; 29; 126 | 14.10 | 41; 47; 82 | 3.30 | 27; 89; 131 | 4.23 | 7; 72; 98 | 4.08 | 31; 62; 85 | 4.86 | 102; 135; 142 | 3.31 |
| 6; 15; 81 | 14.10 | 7; 57; 80 | 3.30 | 27; 116; 131 | 4.23 | 7; 22; 98 | 4.08 | 31; 35; 62 | 4.86 | 41; 95; 136 | 3.31 |
| 15; 29; 68 | 14.10 | 41; 47; 75 | 3.30 | 68; 85; 88 | 4.23 | 62; 89; 98 | 4.08 | 35; 38; 133 | 4.86 | 29; 76; 119 | 3.31 |
| 81; 110; 150 | 12.23 | 41; 47; 73 | 3.30 | 27; 93; 131 | 4.23 | 72; 99; 102 | 4.08 | 31; 35; 69 | 4.86 | 27; 60; 136 | 3.31 |
| 28; 72; 110 | 12.23 | 31; 50; 80 | 3.30 | 23; 118; 131 | 4.23 | 73; 89; 93 | 4.08 | 38; 41; 75 | 4.86 | 36; 136; 143 | 3.31 |
| 74; 110; 151 | 12.23 | 57; 80; 84 | 3.30 | 18; 27; 118 | 4.23 | 61; 63; 98 | 4.08 | 31; 54; 108 | 4.86 | 57; 98; 136 | 3.31 |
| 61; 63; 110 | 12.23 | 50; 88; 114 | 3.30 | 54; 88; 101 | 4.23 | 26; 89; 97 | 4.08 | 37; 74; 108 | 4.86 | 55; 102; 135 | 3.31 |
| 24; 85; 110 | 12.23 | 50; 94; 114 | 3.30 | 26; 84; 147 | 4.23 | 26; 89; 107 | 4.08 | 35; 84; 150 | 4.86 | 29; 53; 97 | 3.31 |
| 26; 65; 110 | 12.23 | 50; 75; 114 | 3.30 | 26; 44; 119 | 4.23 | 77; 98; 122 | 4.08 | 31; 38; 62 | 4.86 | 9; 59; 136 | 3.31 |
| 15; 25; 129 | 12.23 | 15; 60; 125 | 3.30 | 27; 75; 102 | 3.19 | 82; 98; 102 | 4.08 | 31; 70; 75 | 4.86 | 18; 93; 118 | 3.31 |
| 81; 110; 151 | 12.23 | 47; 135; 14 | 3.30 | 17; 118; 131 | 3.19 | 79; 97; 98 | 4.08 | 41; 60; 112 | 4.86 | 53; 63; 118 | 3.31 |
| 14; 37; 110 | 12.23 | 1; 57; 125 | 3.30 | 27; 31; 131 | 3.19 | 17; 89; 150 | 4.08 | 35; 81; 133 | 4.86 | 40; 63; 136 | 3.31 |
| 8; 110; 129 | 12.23 | 8; 50; 128 | 3.30 | 26; 74; 148 | 3.19 | 63; 82; 98 | 4.08 | 35; 85; 150 | 4.86 | 33; 127; 135 | 3.31 |
| 81; 110; 149 | 12.23 | 47; 120; 13 | 3.30 | 26; 68; 74 | 3.19 | 62; 99; 122 | 4.08 | 31; 62; 82 | 4.86 | 59; 80; 127 | 3.31 |
| 15; 84; 126 | 12.23 | 30; 57; 125 | 3.30 | 30; 98; 118 | 3.19 | 17; 89; 107 | 4.08 | 33; 35; 148 | 4.86 | 61; 97; 135 | 3.31 |
| 74; 110; 150 | 12.23 | 50; 80; 133 | 3.30 | 44; 79; 108 | 3.19 | 42; 97; 98 | 4.08 | 35; 38; 129 | 4.86 | 37; 101; 135 | 3.31 |
| 81; 110; 129 | 12.23 | 57; 80; 126 | 3.30 | 64; 88; 101 | 3.19 | 85; 97; 98 | 4.08 | 31; 38; 142 | 4.86 | 59; 127; 138 | 3.31 |
| 74; 110; 120 | 12.23 | 8; 74; 75 | 3.30 | 75; 101; 129 | 3.19 | 90; 99; 122 | 4.08 | 31; 40; 68 | 3.81 | 23; 52; 136 | 3.31 |
| 24; 86; 110 | 12.23 | 47; 85; 132 | 2.16 | 85; 88; 147 | 3.19 | 89; 92; 98 | 4.08 | 31; 40; 70 | 3.81 | 14; 60; 97 | 3.31 |
| 8; 110; 148 | 12.23 | 41; 94; 114 | 2.16 | 27; 98; 131 | 3.19 | 2; 79; 102 | 4.08 | 54; 108; 129 | 3.81 | 27; 59; 95 | 3.31 |
| 8; 110; 147 | 12.23 | 64; 73; 114 | 2.16 | 27; 31; 118 | 3.19 | 21; 98; 101 | 4.08 | 38; 107; 149 | 3.81 | 12; 50; 136 | 3.31 |
| 74; 110; 126 | 12.23 | 47; 52; 74 | 2.16 | 17; 24; 131 | 3.19 | 70; 99; 122 | 4.08 | 35; 74; 133 | 3.81 | 43; 95; 136 | 3.31 |
| 74; 110; 129 | 12.23 | 57; 59; 74 | 2.16 | 46; 88; 100 | 3.19 | 63; 83; 98 | 4.08 | 50; 108; 114 | 3.81 | 127; 132; 135 | 3.31 |
| 74; 110; 132 | 12.23 | 14; 35; 61 | 2.16 | 44; 63; 114 | 3.19 | 72; 97; 98 | 4.08 | 5; 15; 41 | 3.81 | 41; 86; 118 | 3.31 |
| 74; 110; 133 | 12.23 | 47; 85; 134 | 2.16 | 26; 68; 70 | 3.19 | 99; 102; 129 | 4.08 | 75; 107; 149 | 3.81 | 59; 98; 127 | 3.31 |
| 81; 110; 126 | 12.23 | 14; 53; 55 | 2.16 | 68; 84; 88 | 3.19 | 89; 98; 101 | 4.08 | 26; 38; 41 | 3.81 | 27; 43; 97 | 3.31 |
| 81; 110; 132 | 12.23 | 8; 60; 128 | 2.16 | 27; 38; 101 | 3.19 | 28; 98; 122 | 4.08 | 15; 41; 133 | 3.81 | 14; 60; 127 | 3.31 |
| 74; 110; 149 | 12.23 | 14; 53; 57 | 2.16 | 26; 74; 119 | 3.19 | 53; 97; 98 | 4.08 | 31; 64; 88 | 3.81 | 102; 132; 135 | 3.31 |
| 19; 74; 110 | 12.23 | 14; 53; 59 | 2.16 | 27; 74; 97 | 3.19 | 18; 42; 100 | 4.08 | 31; 74; 75 | 3.81 | 63; 125; 135 | 3.31 |
| 81; 110; 133 | 12.23 | 15; 26; 60 | 2.16 | 26; 68; 96 | 3.19 | 39; 89; 97 | 4.08 | 38; 107; 150 | 3.81 | 20; 47; 136 | 3.31 |
| 62; 79; 110 | 12.23 | 14; 35; 53 | 2.16 | 75; 77; 102 | 3.19 | 98; 122; 150 | 4.08 | 41; 50; 112 | 3.81 | 53; 63; 136 | 3.31 |
| 81; 110; 135 | 12.23 | 50; 83; 128 | 2.16 | 98; 118; 131 | 3.19 | 51; 98; 119 | 4.08 | 35; 62; 150 | 3.81 | 60; 101; 119 | 3.31 |
| 74; 110; 140 | 12.23 | 57; 59; 79 | 2.16 | 27; 100; 104 | 3.19 | 63; 70; 98 | 4.08 | 35; 54; 114 | 3.81 | 29; 76; 97 | 3.31 |
| 81; 110; 138 | 12.23 | 29; 31; 50 | 2.16 | 27; 100; 102 | 3.19 | 51; 98; 116 | 4.08 | 35; 84; 108 | 3.81 | 40; 97; 118 | 3.31 |
| 8; 110; 135 | 12.23 | 50; 83; 125 | 2.16 | 81; 99; 102 | 3.19 | 18; 100; 149 | 4.08 | 35; 83; 133 | 3.81 | 20; 126; 135 | 3.31 |
| 74; 110; 146 | 12.23 | 8; 29; 73 | 2.16 | 61; 99; 102 | 3.19 | 98; 122; 141 | 4.08 | 35; 84; 129 | 3.81 | 34; 60; 127 | 3.31 |
| 74; 110; 147 | 12.23 | 13; 30; 53 | 2.16 | 27; 100; 116 | 3.19 | 53; 98; 102 | 4.08 | 41; 64; 112 | 3.81 | 25; 66; 136 | 3.31 |
| 81; 110; 147 | 12.23 | 72; 74; 75 | 2.16 | 24; 117; 147 | 3.19 | 6; 99; 102 | 4.08 | 35; 83; 129 | 3.81 | 59; 81; 127 | 3.31 |
| 78; 93; 110 | 12.23 | 57; 59; 75 | 2.16 | 30; 99; 118 | 3.19 | 89; 92; 126 | 4.08 | 34; 75; 108 | 3.81 | 84; 101; 135 | 3.31 |
| 15; 25; 149 | 12.2 | 20; 24; 50 | 2.16 | 27; 129; 131 | 3.19 | 83; 98; 102 | 4.08 | 15; 62; 140 | 3.81 | 55; 89; 135 | 3.31 |
| 77; 84; 110 | 12.23 | 10; 15; 150 | 2.16 | 26; 131; 147 | 3.19 | 75; 79; 89 | 4.08 | 35; 129; 150 | 3.81 | 24; 64; 119 | 3.31 |
| 15; 25; 138 | 12.23 | 15; 26; 50 | 2.16 | 27; 82; 131 | 3.19 | 12; 97; 98 | 4.08 | 35; 84; 133 | 3.81 | 63; 97; 135 | 3.31 |
| 21; 35; 110 | 12.23 | 26; 55; 63 | 2.16 | 65; 101; 104 | 3.19 | 36; 97; 98 | 4.08 | 29; 35; 114 | 3.81 | 33; 102; 135 | 3.31 |
| 17; 70; 110 | 12.23 | 33; 47; 73 | 2.16 | 28; 30; 147 | 3.19 | 29; 98; 122 | 4.08 | 34; 35; 150 | 3.81 | 24; 64; 97 | 3.31 |
| 15; 26; 47 | 12.23 | 47; 53; 54 | 2.16 | 7; 75; 102 | 3.19 | 76; 79; 89 | 4.08 | 15; 62; 133 | 3.81 | 33; 101; 135 | 3.31 |
| 27; 75; 110 | 12.23 | 15; 59; 143 | 2.16 | 23; 100; 114 | 3.19 | 17; 28; 89 | 4.08 | 31; 37; 69 | 3.81 | 69; 102; 135 | 3.31 |
| 48; 63; 110 | 12.23 | 75; 82; 86 | 2.16 | 24; 98; 131 | 3.19 | 28; 99; 122 | 4.08 | 37; 75; 107 | 3.81 | 59; 96; 127 | 3.31 |
| 15; 26; 54 | 12.23 | 50; 84; 114 | 2.16 | 27; 68; 131 | 3.19 | 42; 99; 122 | 4.08 | 15; 41; 108 | 3.81 | 97; 101; 118 | 3.31 |
| 15; 53; 81 | 12.23 | 47; 53; 74 | 2.16 | 26; 27; 131 | 3.19 | 12; 89; 97 | 4.08 | 35; 37; 129 | 3.81 | 62; 73; 136 | 3.31 |
| 47; 78; 110 | 12.23 | 29; 30; 129 | 2.16 | 81; 98; 102 | 3.19 | 45; 99; 122 | 4.08 | 31; 37; 108 | 3.81 | 33; 97; 135 | 3.31 |
| 63; 90; 110 | 12.23 | 47; 53; 73 | 2.16 | 68; 83; 88 | 3.19 | 68; 99; 122 | 4.08 | 82; 108; 126 | 3.81 | 59; 125; 127 | 3.31 |
| 56; 70; 110 | 12.23 | 47; 53; 72 | 2.16 | 24; 131; 151 | 3.19 | 29; 98; 101 | 4.08 | 38; 40; 150 | 3.81 | 37; 60; 127 | 3.31 |
| 44; 81; 110 | 12.23 | 6; 60; 114 | 2.16 | 68; 88; 101 | 3.19 | 29; 98; 118 | 4.08 | 82; 108; 129 | 3.81 | 63; 98; 135 | 3.31 |
| 15; 26; 65 | 12.23 | 47; 53; 70 | 2.16 | 65; 98; 102 | 3.19 | 62; 98; 122 | 4.08 | 55; 108; 133 | 3.81 | 60; 74; 127 | 3.31 |
| 76; 78; 110 | 12.23 | 47; 68; 72 | 2.16 | 26; 68; 83 | 3.19 | 12; 44; 89 | 4.08 | 6; 31; 35 | 3.81 | 62; 72; 136 | 3.31 |
| 34; 70; 110 | 12.23 | 48; 58; 61 | 2.16 | 27; 74; 118 | 3.19 | 98; 101; 107 | 4.08 | 82; 108; 133 | 3.81 | 24; 59; 119 | 3.31 |
| 3; 86; 110 | 12.23 | 47; 84; 132 | 2.16 | 34; 118; 131 | 3.19 | 17; 69; 89 | 4.08 | 37; 108; 129 | 3.81 | 24; 59; 118 | 3.31 |
| 6; 48; 110 | 12.23 | 47; 53; 75 | 2.16 | 27; 126; 131 | 3.19 | 79; 89; 126 | 4.08 | 35; 38; 148 | 3.81 | 82; 98; 135 | 3.31 |
| 53; 74; 110 | 12.23 | 47; 53; 76 | 2.16 | 65; 99; 102 | 3.19 | 84; 99; 122 | 4.08 | 33; 62; 108 | 3.81 | 29; 127; 135 | 3.31 |
| 24; 81; 112 | 12.23 | 47; 53; 77 | 2.16 | 69; 99; 102 | 3.19 | 18; 83; 89 | 4.08 | 38; 55; 114 | 3.81 | 96; 98; 135 | 3.31 |
| 24; 81; 110 | 12.23 | 47; 53; 84 | 2.16 | 27; 99; 131 | 3.19 | 17; 85; 89 | 4.08 | 35; 74; 146 | 3.81 | 73; 102; 135 | 3.31 |
| 15; 26; 69 | 12.23 | 47; 68; 73 | 2.16 | 27; 89; 119 | 3.19 | 83; 99; 122 | 4.08 | 35; 37; 140 | 3.81 | 48; 101; 118 | 3.31 |
| 37; 61; 110 | 12.23 | 47; 53; 88 | 2.16 | 70; 88; 101 | 3.19 | 83; 97; 98 | 4.08 | 35; 37; 133 | 3.81 | 47; 75; 136 | 3.31 |
| 11; 33; 110 | 12.23 | 47; 53; 87 | 2.16 | 27; 125; 131 | 3.19 | 79; 98; 122 | 4.08 | 31; 40; 79 | 3.81 | 6; 14; 119 | 3.31 |
| 24; 83; 110 | 12.23 | 47; 53; 85 | 2.16 | 51; 100; 114 | 3.19 | 81; 98; 122 | 4.08 | 15; 35; 133 | 3.81 | 27; 42; 70 | 3.31 |
| 23; 47; 110 | 12.23 | 47; 53; 83 | 2.16 | 27; 120; 131 | 3.19 | 22; 98; 101 | 4.08 | 35; 75; 146 | 3.81 | 59; 71; 127 | 3.31 |
| 15; 25; 140 | 12.23 | 47; 53; 78 | 2.16 | 26; 119; 132 | 3.19 | 72; 98; 102 | 4.08 | 15; 35; 132 | 3.81 | 22; 60; 136 | 3.31 |
| 15; 25; 147 | 12.23 | 47; 53; 82 | 2.16 | 27; 116; 141 | 3.19 | 7; 17; 102 | 4.08 | 35; 38; 141 | 3.81 | 3; 47; 136 | 3.31 |
| 15; 25; 148 | 12.23 | 48; 58; 63 | 2.16 | 28; 98; 129 | 3.19 | 15; 99; 101 | 4.08 | 35; 75; 149 | 3.81 | 11; 76; 96 | 3.31 |
| 102; 110; 111 | 12.23 | 47; 53; 81 | 2.16 | 27; 93; 119 | 3.19 | 38; 97; 98 | 4.08 | 38; 40; 147 | 3.81 | 71; 101; 135 | 3.31 |
| 15; 25; 110 | 12.23 | 47; 53; 80 | 2.16 | 3; 28; 100 | 3.19 | 93; 98; 102 | 4.08 | 37; 38; 135 | 3.81 | 26; 78; 118 | 3.31 |
| 15; 25; 150 | 12.23 | 1; 29; 47 | 2.16 | 84; 88; 101 | 3.19 | 72; 98; 116 | 4.08 | 74; 108; 125 | 3.81 | 62; 63; 136 | 3.31 |
| 19; 72; 110 | 12.23 | 47; 53; 79 | 2.16 | 27; 30; 131 | 3.19 | 7; 70; 98 | 4.08 | 31; 37; 82 | 3.81 | 48; 101; 135 | 3.31 |
| 17; 71; 110 | 12.23 | 48; 58; 60 | 2.16 | 28; 98; 131 | 3.19 | 22; 61; 98 | 4.08 | 33; 35; 141 | 3.81 | 98; 101; 135 | 3.31 |
| 15; 26; 28 | 12.23 | 47; 53; 69 | 2.16 | 7; 68; 102 | 3.19 | 64; 99; 122 | 4.08 | 6; 31; 62 | 3.81 | 59; 77; 127 | 3.31 |
| 15; 26; 30 | 12.23 | 8; 53; 59 | 2.16 | 27; 116; 149 | 3.19 | 69; 98; 122 | 4.08 | 35; 37; 108 | 3.81 | 62; 75; 136 | 3.31 |
| 15; 26; 33 | 12.23 | 47; 53; 68 | 2.16 | 83; 85; 88 | 3.19 | 15; 98; 101 | 4.08 | 15; 38; 41 | 3.81 | 73; 101; 135 | 3.31 |
| 24; 84; 110 | 12.23 | 47; 85; 142 | 2.16 | 24; 116; 131 | 3.19 | 18; 85; 89 | 4.08 | 38; 55; 108 | 3.81 | 73; 101; 132 | 3.31 |
| 1; 55; 110 | 12.23 | 47; 68; 69 | 2.16 | 46; 100; 115 | 3.19 | 89; 95; 107 | 4.08 | 31; 37; 75 | 3.81 | 13; 40; 136 | 3.31 |
| 15; 53; 110 | 12.23 | 33; 41; 47 | 2.16 | 27; 92; 131 | 3.19 | 63; 68; 98 | 4.08 | 31; 37; 74 | 3.81 | 76; 125; 126 | 3.31 |
| 15; 26; 37 | 12.23 | 13; 30; 41 | 2.16 | 26; 64; 147 | 3.19 | 22; 68; 98 | 4.08 | 36; 74; 108 | 3.81 | 37; 62; 136 | 3.31 |
| 15; 26; 41 | 12.23 | 47; 84; 142 | 2.16 | 26; 76; 101 | 3.19 | 29; 83; 89 | 4.08 | 40; 114; 150 | 3.81 | 60; 99; 136 | 3.31 |
| 26; 66; 110 | 12.23 | 57; 59; 72 | 2.16 | 83; 88; 101 | 3.19 | 2; 26; 101 | 4.08 | 31; 37; 83 | 3.81 | 60; 95; 127 | 3.31 |
| 8; 110; 149 | 12.23 | 31; 41; 53 | 2.16 | 7; 102; 149 | 3.19 | 69; 99; 122 | 4.08 | 34; 37; 38 | 3.81 | 50; 125; 127 | 3.31 |
| 63; 93; 110 | 12.23 | 47; 84; 149 | 2.16 | 70; 74; 88 | 3.19 | 72; 98; 122 | 4.08 | 33; 38; 85 | 3.81 | 96; 118; 135 | 3.31 |

| | | | | | | | | Retinal | | | |
|---|---|---|---|---|---|---|---|---|---|---|---|
| PDE1C_ACSM3 | | Parietal and | | Photoreceptor | | Retinal | | progenitors and | | SATB2_LRRC7 | |
| positive cells | | chief cells | | cells | | pigment cells | | Muller glia | | positive cells | |
| Clusters | Score | Clusters | Score | Clusters | Score | Clusters | Score | Clusters | Score | Clusters | Score |
| 10; 31; 49 | 7.45 | 75; 126; 133 | 3.30 | 52; 123; 125 | 4.13 | 22; 61; 147 | 3.36 | 73; 98; 135 | 9.08 | 24; 40; 102 | 5.58 |
| 7; 31; 49 | 7.45 | 54; 99; 129 | 2.16 | 54; 123; 125 | 2.96 | 22; 61; 148 | 3.36 | 96; 98; 135 | 9.08 | 40; 102; 128 | 5.58 |
| 31; 49; 143 | 7.45 | 18; 40; 44 | 2.16 | 70; 133; 135 | 2.96 | 4; 20; 22 | 3.36 | 97; 98; 135 | 7.70 | 39; 98; 102 | 5.58 |
| 31; 49; 54 | 7.45 | 54; 99; 150 | 2.16 | 54; 107; 134 | 2.96 | 22; 61; 149 | 3.36 | 83; 98; 135 | 7.70 | 12; 40; 127 | 4.39 |
| 31; 49; 62 | 7.45 | 54; 99; 149 | 2.16 | 82; 86; 133 | 2.96 | 22; 34; 70 | 3.36 | 84; 98; 135 | 7.70 | 39; 91; 102 | 4.39 |
| 31; 49; 75 | 7.45 | 39; 82; 126 | 2.16 | 97; 133; 135 | 2.96 | 22; 61; 134 | 3.36 | 82; 98; 135 | 7.70 | 12; 40; 102 | 4.39 |
| 49; 81; 133 | 6.19 | 39; 126; 138 | 2.16 | 97; 133; 134 | 2.96 | 22; 61; 83 | 3.36 | 26; 86; 97 | 6.40 | 40; 86; 97 | 4.39 |
| 47; 49; 109 | 6.19 | 39; 61; 126 | 2.16 | 54; 133; 134 | 2.96 | 82; 86; 133 | 3.36 | 28; 98; 135 | 6.40 | 24; 39; 102 | 4.39 |
| 18; 50; 109 | 6.19 | 4; 39; 126 | 2.16 | 54; 133; 135 | 2.96 | 22; 25; 61 | 3.36 | 10; 27; 98 | 6.40 | 39; 102; 150 | 4.39 |
| 23; 54; 62 | 6.19 | 5; 39; 127 | 2.16 | 54; 126; 135 | 2.96 | 1; 22; 61 | 3.36 | 29; 98; 126 | 6.40 | 28; 48; 102 | 4.39 |
| 31; 50; 70 | 6.19 | 5; 39; 126 | 2.16 | 54; 127; 135 | 2.96 | 22; 34; 61 | 3.36 | 27; 84; 98 | 6.40 | 39; 102; 126 | 4.39 |
| 25; 49; 69 | 6.19 | 62; 129; 133 | 2.16 | 30; 49; 133 | 2.96 | 22; 34; 150 | 3.36 | 27; 72; 98 | 6.40 | 39; 102; 135 | 4.39 |
| 25; 62; 108 | 6.19 | 8; 39; 138 | 2.16 | 8; 60; 123 | 2.96 | 4; 22; 101 | 3.36 | 29; 98; 119 | 6.40 | 39; 102; 141 | 4.39 |
| 22; 50; 109 | 6.19 | 54; 99; 107 | 2.16 | 30; 44; 125 | 2.96 | 22; 34; 81 | 3.36 | 70; 98; 135 | 6.40 | 39; 102; 142 | 4.39 |
| 50; 54; 109 | 6.19 | 39; 126; 129 | 2.16 | 79; 123; 125 | 2.96 | 22; 34; 148 | 3.36 | 29; 98; 118 | 6.40 | 39; 102; 145 | 4.39 |
| 31; 49; 69 | 6.19 | 39; 62; 138 | 2.16 | 79; 123; 137 | 2.96 | 20; 62; 70 | 2.50 | 28; 98; 118 | 6.40 | 39; 86; 97 | 4.39 |
| 31; 49; 83 | 6.19 | 18; 40; 129 | 2.16 | 54; 64; 127 | 2.96 | 72; 82; 134 | 2.50 | 27; 63; 98 | 6.40 | 20; 39; 102 | 4.39 |
| 31; 49; 82 | 6.19 | 62; 79; 133 | 2.16 | 54; 86; 134 | 2.96 | 22; 34; 43 | 2.50 | 28; 98; 108 | 6.40 | 27; 60; 102 | 4.39 |
| 31; 49; 70 | 6.19 | 54; 127; 133 | 2.16 | 54; 86; 123 | 2.96 | 5; 20; 61 | 2.50 | 27; 44; 98 | 6.40 | 39; 72; 102 | 4.39 |
| 31; 49; 72 | 6.19 | 39; 126; 150 | 2.16 | 81; 123; 125 | 2.96 | 4; 22; 28 | 2.50 | 60; 98; 118 | 6.40 | 39; 72; 97 | 4.39 |
| 50; 62; 109 | 6.19 | 39; 126; 151 | 2.16 | 13; 70; 133 | 2.96 | 4; 22; 25 | 2.50 | 60; 98; 119 | 6.40 | 18; 27; 102 | 4.39 |
| 25; 49; 85 | 6.19 | 18; 40; 127 | 2.16 | 30; 41; 127 | 2.96 | 25; 61; 79 | 2.50 | 78; 98; 118 | 6.40 | 39; 63; 102 | 4.39 |
| 50; 72; 109 | 6.19 | 18; 40; 128 | 2.16 | 30; 41; 126 | 2.96 | 25; 61; 85 | 2.50 | 26; 89; 97 | 6.40 | 39; 83; 102 | 4.39 |
| 28; 50; 109 | 6.19 | 62; 126; 135 | 2.16 | 30; 41; 101 | 2.96 | 25; 61; 81 | 2.50 | 76; 98; 135 | 6.40 | 39; 56; 97 | 4.39 |
| 10; 11; 64 | 6.19 | 62; 126; 133 | 2.16 | 13; 68; 133 | 2.96 | 25; 61; 82 | 2.50 | 63; 98; 135 | 6.40 | 39; 85; 102 | 4.39 |
| 5; 50; 109 | 6.19 | 70; 126; 133 | 2.16 | 54; 75; 123 | 2.96 | 25; 61; 63 | 2.50 | 84; 98; 118 | 6.40 | 18; 39; 113 | 4.39 |
| 18; 31; 50 | 6.19 | 62; 75; 133 | 2.16 | 30; 101; 134 | 2.96 | 61; 128; 151 | 2.50 | 27; 68; 98 | 6.40 | 40; 95; 102 | 4.39 |
| 18; 31; 49 | 6.19 | 40; 88; 128 | 2.16 | 8; 123; 125 | 2.96 | 22; 34; 82 | 2.50 | 98; 132; 135 | 6.40 | 40; 98; 102 | 4.39 |
| 31; 50; 85 | 6.19 | 44; 54; 99 | 2.16 | 69; 133; 135 | 2.96 | 22; 34; 79 | 2.50 | 27; 82; 98 | 6.40 | 40; 97; 113 | 4.39 |
| 25; 56; 108 | 6.19 | 70; 99; 126 | 2.16 | 8; 123; 137 | 2.96 | 110; 140; 151 | 2.50 | 27; 81; 98 | 6.40 | 40; 63; 102 | 3.31 |
| 5; 18; 31 | 6.19 | 8; 39; 126 | 2.16 | 86; 101; 134 | 2.96 | 110; 141; 151 | 2.50 | 98; 135; 142 | 6.40 | 40; 62; 102 | 3.31 |
| 31; 49; 150 | 6.19 | 54; 69; 100 | 2.16 | 6; 30; 123 | 2.96 | 22; 84; 134 | 2.50 | 26; 98; 118 | 6.40 | 36; 39; 98 | 3.31 |
| 44; 50; 109 | 6.19 | 18; 40; 126 | 2.16 | 86; 123; 137 | 2.96 | 110; 142; 151 | 2.50 | 26; 98; 119 | 6.40 | 36; 39; 102 | 3.31 |
| 31; 44; 49 | 6.19 | 54; 69; 99 | 2.16 | 41; 105; 137 | 2.96 | 110; 143; 151 | 2.50 | 27; 76; 98 | 6.40 | 36; 39; 127 | 3.31 |
| 10; 25; 49 | 6.19 | 41; 65; 129 | 2.16 | 86; 123; 125 | 2.96 | 5; 20; 101 | 2.50 | 27; 75; 98 | 6.40 | 39; 54; 102 | 3.31 |
| 18; 31; 62 | 6.19 | 18; 39; 141 | 2.16 | 86; 96; 133 | 2.96 | 9; 22; 101 | 2.50 | 26; 93; 97 | 6.40 | 18; 39; 97 | 3.31 |
| 10; 25; 62 | 6.19 | 18; 39; 151 | 2.16 | 69; 123; 125 | 2.96 | 2; 95; 139 | 2.50 | 27; 65; 98 | 6.40 | 36; 40; 102 | 3.31 |
| 49; 85; 103 | 6.19 | 126; 129; 133 | 2.16 | 8; 133; 135 | 2.96 | 23; 101; 133 | 2.50 | 98; 135; 147 | 6.40 | 18; 39; 102 | 3.31 |
| 31; 49; 85 | 6.19 | 40; 128; 129 | 2.16 | 70; 123; 125 | 2.96 | 22; 101; 134 | 2.50 | 68; 98; 135 | 6.40 | 40; 63; 127 | 3.31 |
| 5; 31; 62 | 6.19 | 40; 127; 140 | 2.16 | 50; 126; 127 | 2.96 | 4; 22; 61 | 2.50 | 47; 98; 118 | 6.40 | 40; 98; 107 | 3.31 |
| 23; 62; 64 | 6.19 | 40; 65; 107 | 2.16 | 50; 127; 133 | 2.96 | 6; 22; 25 | 2.50 | 72; 98; 135 | 6.40 | 28; 56; 127 | 3.31 |
| 18; 25; 74 | 6.19 | 62; 100; 126 | 2.16 | 60; 96; 98 | 2.96 | 15; 25; 97 | 2.50 | 27; 70; 98 | 6.40 | 40; 60; 127 | 3.31 |
| 25; 72; 75 | 6.19 | 62; 76; 148 | 2.16 | 30; 128; 137 | 2.96 | 6; 22; 61 | 2.50 | 27; 98; 108 | 6.40 | 22; 39; 119 | 3.31 |
| 25; 49; 108 | 6.19 | 40; 127; 139 | 2.16 | 30; 126; 133 | 2.96 | 6; 22; 83 | 2.50 | 27; 98; 118 | 6.40 | 102; 105; 144 | 3.31 |
| 49; 54; 109 | 6.19 | 88; 133; 150 | 2.16 | 14; 78; 134 | 2.96 | 4; 22; 70 | 2.50 | 27; 73; 98 | 6.40 | 39; 57; 102 | 3.31 |
| 7; 50; 109 | 6.19 | 18; 88; 133 | 2.16 | 74; 97; 132 | 2.96 | 44; 138; 151 | 2.50 | 37; 98; 135 | 6.40 | 40; 60; 102 | 3.31 |
| 25; 49; 150 | 6.19 | 18; 40; 89 | 2.16 | 60; 107; 123 | 2.96 | 2; 49; 97 | 2.50 | 27; 98; 146 | 6.40 | 22; 40; 95 | 3.31 |
| 25; 49; 147 | 6.19 | 41; 126; 138 | 2.16 | 30; 133; 134 | 2.96 | 9; 27; 107 | 2.50 | 79; 98; 135 | 6.40 | 27; 74; 97 | 3.31 |
| 23; 62; 143 | 5.02 | 79; 99; 126 | 2.16 | 8; 30; 123 | 2.96 | 43; 61; 151 | 2.50 | 27; 85; 98 | 6.40 | 39; 56; 119 | 3.31 |
| 25; 74; 75 | 5.02 | 40; 127; 138 | 2.16 | 14; 123; 125 | 2.96 | 9; 27; 97 | 2.50 | 27; 79; 98 | 6.40 | 39; 56; 102 | 3.31 |
| 23; 62; 75 | 5.02 | 40; 127; 137 | 2.16 | 60; 89; 123 | 2.96 | 22; 28; 61 | 2.50 | 37; 98; 118 | 6.40 | 22; 40; 102 | 3.31 |
| 23; 38; 62 | 5.02 | 54; 82; 99 | 2.16 | 30; 123; 125 | 2.96 | 22; 28; 34 | 2.50 | 27; 98; 119 | 6.40 | 22; 40; 127 | 3.31 |
| 23; 62; 73 | 5.02 | 62; 88; 125 | 2.16 | 60; 118; 123 | 2.96 | 9; 27; 88 | 2.50 | 98; 101; 135 | 6.40 | 56; 72; 118 | 3.31 |
| 50; 70; 99 | 5.02 | 40; 128; 150 | 2.16 | 52; 123; 133 | 2.96 | 22; 28; 142 | 2.50 | 30; 98; 135 | 5.19 | 40; 64; 102 | 3.31 |
| 32; 49; 56 | 5.02 | 40; 128; 149 | 2.16 | 14; 40; 125 | 2.96 | 22; 28; 134 | 2.50 | 68; 98; 118 | 5.19 | 40; 97; 147 | 3.31 |
| 18; 20; 74 | 5.02 | 40; 128; 148 | 2.16 | 74; 133; 135 | 2.96 | 20; 61; 82 | 2.50 | 78; 98; 108 | 5.19 | 97; 98; 102 | 3.31 |
| 28; 49; 72 | 5.02 | 41; 65; 89 | 2.16 | 46; 101; 134 | 2.96 | 2; 95; 110 | 2.50 | 7; 27; 98 | 5.19 | 40; 70; 102 | 3.31 |
| 32; 49; 54 | 5.02 | 62; 88; 133 | 2.16 | 60; 138; 140 | 2.96 | 20; 61; 79 | 2.50 | 73; 84; 98 | 5.19 | 40; 70; 127 | 3.31 |
| 15; 27; 50 | 5.02 | 62; 76; 126 | 2.16 | 60; 119; 123 | 2.96 | 34; 43; 143 | 2.50 | 98; 126; 147 | 5.19 | 27; 97; 113 | 3.31 |
| 10; 25; 150 | 5.02 | 39; 65; 126 | 2.16 | 14; 41; 125 | 2.96 | 22; 25; 146 | 2.50 | 27; 87; 98 | 5.19 | 18; 40; 97 | 3.31 |
| 15; 18; 64 | 5.02 | 40; 65; 66 | 2.16 | 14; 41; 137 | 2.96 | 22; 25; 134 | 2.50 | 98; 126; 148 | 5.19 | 24; 58; 102 | 3.31 |
| 23; 56; 62 | 5.02 | 62; 99; 126 | 2.16 | 46; 115; 137 | 2.96 | 22; 61; 85 | 2.50 | 98; 126; 150 | 5.19 | 40; 97; 107 | 3.31 |
| 23; 56; 64 | 5.02 | 40; 128; 138 | 2.16 | 14; 41; 101 | 2.96 | 22; 61; 84 | 2.50 | 98; 129; 132 | 5.19 | 18; 40; 102 | 3.31 |
| 23; 62; 72 | 5.02 | 75; 99; 126 | 2.16 | 14; 57; 137 | 2.96 | 5; 22; 81 | 2.50 | 28; 72; 98 | 5.19 | 40; 69; 102 | 3.31 |
| 10; 49; 96 | 5.02 | 40; 128; 137 | 2.16 | 75; 126; 135 | 2.96 | 22; 68; 134 | 2.50 | 44; 73; 98 | 5.19 | 97; 99; 113 | 3.31 |
| 7; 15; 64 | 5.02 | 40; 128; 139 | 2.16 | 60; 123; 125 | 2.96 | 22; 61; 77 | 2.50 | 76; 82; 98 | 5.19 | 40; 65; 127 | 3.31 |
| 7; 15; 62 | 5.02 | 69; 99; 126 | 2.16 | 75; 133; 135 | 2.96 | 27; 82; 135 | 2.50 | 27; 69; 98 | 5.19 | 36; 48; 102 | 3.31 |
| 25; 54; 108 | 5.02 | 62; 70; 133 | 2.16 | 75; 135; 147 | 2.96 | 22; 61; 74 | 2.50 | 28; 37; 98 | 5.19 | 89; 97; 113 | 3.31 |
| 50; 70; 109 | 5.02 | 40; 128; 143 | 2.16 | 75; 135; 148 | 2.96 | 22; 61; 73 | 2.50 | 14; 98; 101 | 5.19 | 40; 64; 127 | 3.31 |
| 49; 83; 103 | 5.02 | 126; 133; 150 | 2.16 | 75; 123; 125 | 2.96 | 22; 61; 69 | 2.50 | 28; 88; 98 | 5.19 | 40; 97; 127 | 3.31 |
| 31; 47; 49 | 5.02 | 39; 65; 129 | 2.16 | 52; 128; 137 | 2.96 | 22; 61; 68 | 2.50 | 10; 98; 135 | 5.19 | 8; 23; 127 | 3.31 |
| 23; 64; 75 | 5.02 | 40; 65; 106 | 2.16 | 75; 97; 132 | 2.96 | 22; 61; 90 | 2.50 | 27; 83; 98 | 5.19 | 39; 52; 97 | 3.31 |
| 26; 49; 76 | 5.02 | 40; 65; 150 | 2.16 | 52; 104; 123 | 2.96 | 22; 61; 114 | 2.50 | 26; 76; 97 | 5.19 | 40; 65; 102 | 3.31 |
| 5; 50; 111 | 5.02 | 40; 65; 151 | 2.16 | 60; 127; 134 | 2.96 | 22; 61; 101 | 2.50 | 73; 98; 146 | 5.19 | 27; 86; 102 | 3.31 |
| 27; 50; 54 | 5.02 | 40; 44; 151 | 2.16 | 60; 127; 133 | 2.96 | 15; 70; 82 | 2.50 | 73; 75; 98 | 5.19 | 24; 59; 105 | 3.31 |
| 50; 70; 106 | 5.02 | 20; 62; 100 | 2.16 | 74; 123; 125 | 2.96 | 9; 82; 101 | 2.50 | 6; 27; 98 | 5.19 | 24; 59; 102 | 3.31 |
| 23; 64; 74 | 5.02 | 40; 44; 65 | 2.16 | 52; 133; 135 | 2.96 | 44; 82; 101 | 2.50 | 72; 98; 119 | 5.19 | 40; 66; 102 | 3.31 |
| 27; 50; 56 | 5.02 | 18; 40; 65 | 2.16 | 60; 126; 134 | 2.96 | 44; 82; 103 | 2.50 | 37; 98; 119 | 5.19 | 40; 68; 102 | 3.31 |
| 23; 62; 63 | 5.02 | 82; 99; 126 | 2.16 | 14; 123; 137 | 2.96 | 49; 110; 123 | 2.50 | 21; 98; 117 | 5.19 | 40; 68; 127 | 3.31 |
| 28; 50; 85 | 5.02 | 5; 39; 138 | 2.16 | 78; 123; 137 | 2.96 | 106; 110; 123 | 2.50 | 72; 97; 98 | 5.19 | 27; 86; 97 | 3.31 |
| 23; 64; 72 | 5.02 | 88; 126; 133 | 2.16 | 30; 82; 133 | 2.96 | 25; 81; 151 | 2.50 | 72; 98; 134 | 5.19 | 27; 98; 102 | 3.31 |
| 50; 84; 99 | 5.02 | 69; 126; 132 | 2.16 | 78; 86; 132 | 2.96 | 106; 110; 151 | 2.50 | 29; 76; 98 | 5.19 | 39; 72; 119 | 3.31 |
| 50; 54; 99 | 5.02 | 40; 126; 128 | 2.16 | 53; 126; 133 | 2.96 | 8; 22; 81 | 2.50 | 29; 90; 98 | 5.19 | 40; 98; 138 | 3.31 |
| 10; 49; 99 | 5.02 | 40; 127; 128 | 2.16 | 78; 133; 135 | 2.96 | 4; 49; 104 | 2.50 | 45; 98; 135 | 5.19 | 98; 102; 107 | 3.31 |
| 10; 25; 70 | 5.02 | 62; 88; 100 | 2.16 | 98; 123; 137 | 2.96 | 44; 79; 134 | 2.50 | 47; 98; 101 | 5.19 | 40; 50; 127 | 3.31 |
| 20; 49; 108 | 5.02 | 18; 40; 83 | 2.16 | 123; 127; 133 | 2.96 | 22; 62; 149 | 2.50 | 45; 98; 119 | 5.19 | 24; 101; 119 | 3.31 |
| 22; 50; 106 | 5.02 | 40; 126; 140 | 2.16 | 13; 126; 133 | 2.96 | 49; 97; 134 | 2.50 | 45; 98; 118 | 5.19 | 28; 75; 102 | 3.31 |
| 49; 56; 109 | 5.02 | 40; 126; 139 | 2.16 | 14; 127; 135 | 2.96 | 49; 97; 103 | 2.50 | 18; 98; 118 | 5.19 | 24; 30; 113 | 3.31 |
| 25; 70; 75 | 5.02 | 40; 126; 138 | 2.16 | 14; 126; 133 | 2.96 | 49; 97; 126 | 2.50 | 18; 98; 135 | 5.19 | 40; 98; 127 | 3.31 |
| 49; 56; 111 | 5.02 | 40; 65; 129 | 2.16 | 14; 128; 137 | 2.96 | 10; 95; 138 | 2.50 | 45; 97; 98 | 5.19 | 24; 101; 102 | 3.31 |
| 50; 107; 108 | 5.02 | 18; 40; 82 | 2.16 | 60; 81; 123 | 2.96 | 44; 79; 132 | 2.50 | 47; 98; 119 | 5.19 | 39; 70; 102 | 3.31 |
| 49; 73; 81 | 5.02 | 40; 97; 128 | 2.16 | 60; 79; 123 | 2.96 | 8; 22; 101 | 2.50 | 37; 97; 98 | 5.19 | 39; 70; 97 | 3.31 |
| 23; 64; 129 | 5.02 | 40; 118; 139 | 2.16 | 123; 126; 127 | 2.96 | 44; 79; 151 | 2.50 | 51; 98; 148 | 5.19 | 40; 52; 102 | 3.31 |
| 50; 82; 109 | 5.02 | 18; 62; 100 | 2.16 | 47; 127; 133 | 2.96 | 5; 22; 133 | 2.50 | 17; 98; 146 | 5.19 | 98; 102; 120 | 3.31 |
| 17; 50; 114 | 5.02 | 18; 62; 99 | 2.16 | 60; 70; 123 | 2.96 | 22; 62; 68 | 2.50 | 14; 98; 130 | 5.19 | 39; 59; 102 | 3.31 |
| 15; 25; 69 | 5.02 | 18; 40; 150 | 2.16 | 30; 89; 134 | 2.96 | 22; 61; 150 | 2.50 | 98; 118; 126 | 5.19 | 27; 57; 102 | 3.31 |
| 13; 23; 62 | 5.02 | 18; 40; 151 | 2.16 | 30; 89; 123 | 2.96 | 42; 97; 138 | 2.50 | 27; 98; 148 | 5.19 | 20; 40; 127 | 3.31 |
| 11; 25; 50 | 5.02 | 18; 40; 148 | 2.16 | 50; 101; 127 | 2.96 | 15; 69; 82 | 2.50 | 76; 98; 118 | 5.19 | 18; 27; 138 | 3.31 |
| 31; 44; 62 | 5.02 | 75; 88; 133 | 2.16 | 89; 123; 137 | 2.96 | 42; 95; 138 | 2.50 | 63; 98; 108 | 5.19 | 39; 74; 127 | 3.31 |
| 49; 56; 99 | 5.02 | 40; 118; 128 | 2.16 | 68; 74; 135 | 2.96 | 22; 62; 84 | 2.50 | 84; 98; 119 | 5.19 | 39; 74; 102 | 3.31 |

| SKOR2_NPSR1 | | STC2_TLX1 | | | | | | Skeletal | | Smooth | |
|---|---|---|---|---|---|---|---|---|---|---|---|
| positive cells | | positive cells | | Satellite cells | | Schwann cells | | muscle cells | | muscle cells | |
| Clusters | Score | Clusters | Score | Clusters | Score | Clusters | Score | Clusters | Score | Clusters | Score |
| 34; 54; 118 | 3.12 | 69; 144; 149 | 6.00 | 64; 97; 145 | 7.83 | 8; 77; 114 | 8.66 | 10; 63; 101 | 8.28 | 72; 113; 145 | 11.74 |
| 9; 83; 146 | 3.12 | 119; 141; 144 | 6.00 | 74; 101; 145 | 6.62 | 4; 120; 146 | 8.66 | 55; 82; 101 | 8.28 | 121; 122; 141 | 11.74 |
| 9; 86; 146 | 3.12 | 6; 69; 144 | 6.00 | 63; 72; 145 | 6.62 | 72; 83; 114 | 8.66 | 20; 44; 101 | 8.28 | 53; 113; 145 | 11.74 |
| 9; 146; 147 | 3.12 | 36; 79; 144 | 6.00 | 53; 101; 145 | 6.62 | 81; 133; 149 | 8.66 | 20; 65; 101 | 8.28 | 9; 113; 145 | 11.74 |
| 41; 72; 118 | 3.12 | 62; 144; 149 | 6.00 | 69; 78; 144 | 6.62 | 90; 114; 149 | 7.43 | 7; 54; 101 | 8.28 | 48; 113; 145 | 11.74 |
| 9; 68; 146 | 3.12 | 61; 69; 144 | 6.00 | 98; 101; 145 | 6.62 | 68; 114; 145 | 7.43 | 7; 10; 101 | 8.28 | 45; 113; 145 | 11.74 |
| 9; 75; 146 | 3.12 | 36; 70; 144 | 6.00 | 25; 145; 150 | 6.62 | 125; 141; 145 | 7.43 | 10; 20; 101 | 8.28 | 82; 113; 145 | 10.50 |
| 42; 65; 118 | 3.12 | 141; 144; 147 | 6.00 | 90; 101; 145 | 6.62 | 80; 138; 150 | 7.43 | 10; 70; 101 | 7.01 | 55; 113; 145 | 10.50 |
| 41; 75; 118 | 3.12 | 141; 144; 146 | 6.00 | 75; 101; 145 | 6.62 | 80; 138; 149 | 7.43 | 22; 64; 101 | 7.01 | 33; 122; 145 | 10.50 |
| 9; 33; 146 | 3.12 | 120; 141; 144 | 6.00 | 54; 101; 145 | 6.62 | 80; 138; 148 | 7.43 | 55; 63; 101 | 7.01 | 121; 122; 145 | 10.50 |
| 58; 90; 127 | 3.12 | 69; 70; 144 | 6.00 | 63; 101; 145 | 6.62 | 4; 148; 149 | 7.43 | 10; 73; 101 | 7.01 | 37; 121; 149 | 10.50 |
| 65; 108; 146 | 3.12 | 6; 36; 144 | 6.00 | 38; 101; 145 | 6.62 | 17; 114; 149 | 7.43 | 22; 82; 101 | 7.01 | 60; 122; 145 | 10.50 |
| 34; 70; 118 | 3.12 | 69; 83; 144 | 6.00 | 77; 101; 145 | 6.62 | 83; 132; 149 | 7.43 | 10; 90; 101 | 7.01 | 29; 122; 145 | 10.50 |
| 58; 110; 127 | 3.12 | 6; 144; 149 | 6.00 | 83; 132; 149 | 6.62 | 8; 114; 141 | 7.43 | 53; 93; 101 | 7.01 | 72; 122; 145 | 10.50 |
| 9; 108; 146 | 3.12 | 36; 84; 144 | 6.00 | 83; 132; 145 | 6.62 | 90; 138; 145 | 7.43 | 10; 14; 101 | 7.01 | 106; 122; 145 | 10.50 |
| 9; 79; 146 | 3.12 | 62; 83; 144 | 6.00 | 29; 101; 145 | 6.62 | 63; 114; 149 | 7.43 | 75; 90; 101 | 7.01 | 10; 122; 145 | 10.50 |
| 95; 103; 127 | 2.04 | 62; 79; 144 | 6.00 | 14; 101; 145 | 6.62 | 68; 80; 145 | 7.43 | 10; 35; 101 | 7.01 | 121; 122; 144 | 10.50 |
| 5; 92; 108 | 2.04 | 62; 69; 144 | 6.00 | 67; 119; 144 | 6.62 | 68; 80; 138 | 7.43 | 54; 81; 101 | 7.01 | 83; 113; 145 | 10.50 |
| 63; 97; 127 | 2.04 | 62; 70; 144 | 6.00 | 81; 101; 145 | 6.62 | 90; 148; 149 | 7.43 | 14; 83; 101 | 7.01 | 85; 113; 145 | 10.50 |
| 4; 127; 142 | 2.04 | 36; 62; 144 | 6.00 | 61; 94; 138 | 6.62 | 61; 90; 114 | 7.43 | 7; 25; 101 | 7.01 | 9; 122; 145 | 10.50 |
| 65; 118; 127 | 2.04 | 62; 68; 144 | 6.00 | 5; 97; 144 | 6.62 | 4; 146; 149 | 7.43 | 70; 90; 101 | 7.01 | 64; 122; 145 | 10.50 |
| 45; 84; 118 | 2.04 | 68; 69; 144 | 6.00 | 62; 82; 145 | 6.62 | 90; 114; 142 | 7.43 | 8; 62; 101 | 7.01 | 13; 113; 145 | 10.50 |
| 33; 83; 127 | 2.04 | 6; 138; 144 | 4.86 | 62; 72; 145 | 6.62 | 77; 121; 141 | 7.43 | 61; 90; 101 | 7.01 | 62; 113; 145 | 10.50 |
| 55; 108; 118 | 2.04 | 79; 85; 144 | 4.86 | 65; 86; 145 | 6.62 | 6; 114; 145 | 7.43 | 7; 20; 101 | 7.01 | 6; 113; 145 | 10.50 |
| 58; 75; 127 | 2.04 | 54; 83; 144 | 4.86 | 62; 138; 145 | 6.62 | 25; 114; 149 | 7.43 | 1; 10; 101 | 7.01 | 38; 113; 145 | 10.50 |
| 102; 110; 127 | 2.04 | 8; 74; 144 | 4.86 | 55; 101; 145 | 6.62 | 129; 146; 149 | 7.43 | 82; 90; 101 | 7.01 | 56; 122; 145 | 10.50 |
| 44; 58; 127 | 2.04 | 68; 126; 144 | 4.86 | 66; 86; 145 | 6.62 | 69; 148; 149 | 7.43 | 10; 88; 101 | 7.01 | 63; 113; 145 | 10.50 |
| 33; 84; 127 | 2.04 | 68; 129; 144 | 4.86 | 56; 101; 145 | 6.62 | 68; 133; 149 | 7.43 | 20; 101; 134 | 7.01 | 114; 122; 145 | 10.50 |
| 9; 61; 118 | 2.04 | 10; 138; 144 | 4.86 | 56; 101; 144 | 6.62 | 83; 133; 149 | 7.43 | 7; 90; 101 | 7.01 | 22; 113; 145 | 10.50 |
| 4; 33; 127 | 2.04 | 68; 114; 144 | 4.86 | 101; 142; 145 | 6.62 | 8; 148; 149 | 7.43 | 10; 55; 101 | 7.01 | 5; 113; 145 | 10.50 |
| 45; 86; 118 | 2.04 | 114; 144; 149 | 4.86 | 101; 135; 145 | 6.62 | 68; 77; 138 | 7.43 | 83; 90; 101 | 7.01 | 122; 138; 141 | 10.50 |
| 90; 103; 127 | 2.04 | 79; 120; 144 | 4.86 | 101; 134; 145 | 6.62 | 90; 114; 145 | 7.43 | 20; 53; 101 | 7.01 | 42; 113; 145 | 10.50 |
| 44; 55; 118 | 2.04 | 68; 119; 144 | 4.86 | 79; 101; 145 | 6.62 | 97; 141; 146 | 7.43 | 20; 76; 101 | 7.01 | 47; 113; 145 | 10.50 |
| 45; 90; 128 | 2.04 | 68; 120; 144 | 4.86 | 49; 101; 144 | 6.62 | 83; 114; 145 | 7.43 | 25; 82; 101 | 7.01 | 76; 113; 145 | 10.50 |
| 48; 74; 127 | 2.04 | 68; 132; 144 | 4.86 | 7; 97; 145 | 5.49 | 6; 114; 150 | 7.43 | 76; 90; 101 | 7.01 | 4; 113; 145 | 10.50 |
| 9; 62; 118 | 2.04 | 54; 84; 144 | 4.86 | 73; 101; 144 | 5.49 | 69; 80; 138 | 7.43 | 53; 101; 129 | 7.01 | 122; 138; 145 | 10.50 |
| 5; 88; 118 | 2.04 | 6; 144; 146 | 4.86 | 73; 101; 145 | 5.49 | 114; 125; 141 | 7.43 | 20; 70; 101 | 7.01 | 8; 113; 145 | 10.50 |
| 13; 127; 142 | 2.04 | 6; 144; 147 | 4.86 | 42; 101; 145 | 5.49 | 96; 146; 148 | 7.43 | 10; 75; 101 | 7.01 | 60; 122; 144 | 10.50 |
| 39; 65; 97 | 2.04 | 69; 72; 144 | 4.86 | 27; 80; 144 | 5.49 | 96; 146; 149 | 7.43 | 6; 20; 101 | 7.01 | 113; 133; 145 | 10.50 |
| 9; 62; 127 | 2.04 | 79; 138; 144 | 4.86 | 66; 134; 145 | 5.49 | 80; 83; 138 | 7.43 | 7; 22; 101 | 7.01 | 44; 113; 145 | 10.50 |
| 60; 90; 127 | 2.04 | 25; 69; 144 | 4.86 | 42; 87; 145 | 5.49 | 96; 148; 149 | 7.43 | 10; 76; 101 | 7.01 | 120; 122; 145 | 10.50 |
| 9; 110; 127 | 2.04 | 53; 121; 144 | 4.86 | 42; 78; 145 | 5.49 | 96; 149; 150 | 7.43 | 22; 61; 101 | 7.01 | 45; 122; 145 | 10.50 |
| 58; 78; 127 | 2.04 | 79; 141; 144 | 4.86 | 66; 119; 144 | 5.49 | 5; 138; 142 | 7.43 | 20; 54; 101 | 7.01 | 7; 113; 145 | 10.50 |
| 7; 63; 127 | 2.04 | 69; 74; 144 | 4.86 | 69; 86; 145 | 5.49 | 53; 83; 114 | 7.43 | 22; 63; 101 | 7.01 | 54; 113; 145 | 10.50 |
| 5; 86; 127 | 2.04 | 69; 75; 144 | 4.86 | 74; 78; 144 | 5.49 | 5; 133; 145 | 7.43 | 7; 63; 101 | 7.01 | 20; 122; 145 | 10.50 |
| 5; 6; 127 | 2.04 | 69; 76; 144 | 4.86 | 72; 79; 145 | 5.49 | 80; 83; 145 | 7.43 | 20; 69; 101 | 7.01 | 67; 122; 145 | 10.50 |
| 44; 54; 127 | 2.04 | 54; 85; 144 | 4.86 | 88; 101; 145 | 5.49 | 4; 114; 145 | 7.43 | 55; 90; 101 | 7.01 | 67; 122; 144 | 10.50 |
| 39; 64; 128 | 2.04 | 68; 138; 144 | 4.86 | 27; 134; 145 | 5.49 | 80; 85; 149 | 7.43 | 10; 54; 101 | 7.01 | 86; 113; 145 | 10.50 |
| 20; 64; 127 | 2.04 | 6; 141; 144 | 4.86 | 66; 101; 145 | 5.49 | 10; 90; 114 | 7.43 | 76; 92; 101 | 7.01 | 7; 122; 145 | 10.50 |
| 65; 72; 104 | 2.04 | 79; 129; 144 | 4.86 | 41; 101; 145 | 5.49 | 70; 80; 145 | 7.43 | 10; 68; 101 | 7.01 | 10; 113; 145 | 10.50 |
| 39; 64; 118 | 2.04 | 68; 144; 149 | 4.86 | 66; 95; 145 | 5.49 | 72; 83; 146 | 7.43 | 10; 62; 101 | 7.01 | 107; 122; 145 | 9.30 |
| 13; 41; 89 | 2.04 | 68; 141; 144 | 4.86 | 87; 101; 145 | 5.49 | 72; 83; 148 | 7.43 | 84; 90; 101 | 7.01 | 71; 121; 145 | 9.30 |
| 7; 90; 127 | 2.04 | 68; 144; 146 | 4.86 | 87; 101; 144 | 5.49 | 70; 114; 145 | 7.43 | 10; 101; 107 | 7.01 | 6; 122; 144 | 9.30 |
| 74; 103; 127 | 2.04 | 79; 132; 144 | 4.86 | 107; 142; 145 | 5.49 | 53; 80; 149 | 7.43 | 10; 82; 101 | 7.01 | 72; 122; 142 | 9.30 |
| 72; 73; 108 | 2.04 | 68; 144; 147 | 4.86 | 74; 142; 145 | 5.49 | 85; 114; 148 | 7.43 | 10; 45; 101 | 7.01 | 6; 122; 145 | 9.30 |
| 83; 108; 127 | 2.04 | 8; 75; 144 | 4.86 | 27; 71; 145 | 5.49 | 53; 80; 148 | 7.43 | 10; 22; 101 | 7.01 | 7; 122; 141 | 9.30 |
| 5; 90; 127 | 2.04 | 68; 70; 144 | 4.86 | 57; 101; 145 | 5.49 | 69; 114; 145 | 7.43 | 6; 22; 101 | 7.01 | 47; 113; 144 | 9.30 |
| 5; 90; 118 | 2.04 | 41; 138; 144 | 4.86 | 89; 101; 145 | 5.49 | 90; 138; 142 | 7.43 | 54; 92; 101 | 7.01 | 113; 122; 144 | 9.30 |
| 48; 75; 127 | 2.04 | 114; 118; 144 | 4.86 | 90; 138; 145 | 5.49 | 29; 114; 149 | 7.43 | 10; 101; 129 | 7.01 | 66; 122; 145 | 9.30 |
| 7; 41; 118 | 2.04 | 68; 76; 144 | 4.86 | 90; 138; 142 | 5.49 | 77; 133; 148 | 7.43 | 54; 90; 101 | 7.01 | 39; 122; 145 | 9.30 |
| 39; 65; 112 | 2.04 | 1; 141; 144 | 4.86 | 90; 134; 145 | 5.49 | 114; 134; 145 | 7.43 | 10; 101; 126 | 7.01 | 43; 122; 144 | 9.30 |
| 20; 65; 127 | 2.04 | 68; 79; 144 | 4.86 | 72; 90; 145 | 5.49 | 83; 120; 145 | 7.43 | 10; 85; 101 | 7.01 | 77; 122; 142 | 9.30 |
| 33; 90; 127 | 2.04 | 1; 129; 144 | 4.86 | 56; 118; 144 | 5.49 | 77; 124; 149 | 7.43 | 10; 61; 101 | 7.01 | 14; 122; 145 | 9.30 |
| 39; 65; 118 | 2.04 | 6; 129; 144 | 4.86 | 90; 132; 145 | 5.49 | 76; 148; 149 | 7.43 | 10; 25; 101 | 7.01 | 122; 133; 145 | 9.30 |
| 5; 89; 127 | 2.04 | 1; 144; 146 | 4.86 | 56; 119; 144 | 5.49 | 77; 80; 114 | 7.43 | 10; 84; 101 | 7.01 | 41; 122; 145 | 9.30 |
| 63; 103; 127 | 2.04 | 68; 72; 144 | 4.86 | 90; 129; 145 | 5.49 | 70; 89; 145 | 7.43 | 10; 79; 101 | 7.01 | 61; 121; 149 | 9.30 |
| 39; 65; 128 | 2.04 | 79; 83; 144 | 4.86 | 44; 101; 145 | 5.49 | 6; 77; 138 | 7.43 | 53; 90; 101 | 7.01 | 85; 122; 145 | 9.30 |
| 65; 127; 142 | 2.04 | 53; 144; 146 | 4.86 | 63; 86; 145 | 5.49 | 125; 138; 141 | 7.43 | 7; 55; 101 | 7.01 | 85; 122; 144 | 9.30 |
| 9; 34; 118 | 2.04 | 53; 144; 147 | 4.86 | 44; 101; 144 | 5.49 | 78; 80; 145 | 7.43 | 20; 63; 101 | 7.01 | 60; 139; 144 | 9.30 |
| 9; 38; 81 | 2.04 | 1; 132; 144 | 4.86 | 26; 101; 145 | 5.49 | 5; 114; 145 | 7.43 | 10; 83; 101 | 7.01 | 75; 121; 142 | 9.30 |
| 45; 73; 118 | 2.04 | 79; 82; 144 | 4.86 | 72; 97; 145 | 5.49 | 3; 141; 148 | 7.43 | 10; 101; 150 | 7.01 | 43; 122; 145 | 9.30 |
| 25; 65; 118 | 2.04 | 53; 144; 149 | 4.86 | 44; 97; 144 | 5.49 | 1; 148; 149 | 7.43 | 61; 92; 101 | 5.82 | 23; 113; 145 | 9.30 |
| 28; 39; 118 | 2.04 | 68; 74; 144 | 4.86 | 56; 141; 145 | 5.49 | 89; 114; 149 | 7.43 | 55; 85; 101 | 5.82 | 34; 113; 129 | 9.30 |
| 63; 84; 127 | 2.04 | 68; 75; 144 | 4.86 | 72; 101; 145 | 5.49 | 72; 114; 149 | 7.43 | 10; 101; 148 | 5.82 | 10; 114; 144 | 9.30 |
| 39; 70; 118 | 2.04 | 41; 144; 146 | 4.86 | 72; 107; 145 | 5.49 | 6; 138; 145 | 7.43 | 19; 67; 101 | 5.82 | 22; 71; 144 | 9.30 |
| 44; 63; 127 | 2.04 | 1; 144; 149 | 4.86 | 33; 101; 145 | 5.49 | 6; 114; 120 | 7.43 | 63; 93; 101 | 5.82 | 41; 122; 144 | 9.30 |
| 45; 72; 126 | 2.04 | 8; 69; 144 | 4.86 | 72; 126; 145 | 5.49 | 90; 133; 145 | 7.43 | 10; 64; 101 | 5.82 | 71; 120; 144 | 9.30 |
| 13; 108; 127 | 2.04 | 54; 75; 144 | 4.86 | 72; 132; 145 | 5.49 | 80; 145; 149 | 7.43 | 52; 63; 101 | 5.82 | 23; 113; 144 | 9.30 |
| 45; 72; 147 | 2.04 | 53; 138; 144 | 4.86 | 72; 142; 145 | 5.49 | 6; 138; 149 | 7.43 | 76; 85; 101 | 5.82 | 66; 122; 144 | 9.30 |
| 58; 72; 127 | 2.04 | 68; 108; 144 | 4.86 | 67; 70; 144 | 5.49 | 133; 148; 149 | 7.43 | 1; 101; 150 | 5.82 | 113; 135; 144 | 9.30 |
| 5; 100; 128 | 2.04 | 10; 144; 149 | 4.86 | 10; 101; 145 | 5.49 | 7; 114; 146 | 7.43 | 55; 79; 101 | 5.82 | 85; 122; 141 | 9.30 |
| 5; 100; 127 | 2.04 | 79; 84; 144 | 4.86 | 10; 101; 144 | 5.49 | 80; 147; 149 | 7.43 | 14; 88; 101 | 5.82 | 72; 122; 144 | 9.30 |
| 9; 126; 127 | 2.04 | 54; 79; 144 | 4.86 | 108; 142; 145 | 5.49 | 5; 80; 138 | 7.43 | 92; 101; 129 | 5.82 | 6; 121; 149 | 9.30 |
| 20; 70; 127 | 2.04 | 114; 141; 144 | 4.86 | 28; 78; 144 | 5.49 | 80; 148; 150 | 7.43 | 22; 79; 101 | 5.82 | 36; 113; 145 | 9.30 |
| 45; 75; 118 | 2.04 | 8; 72; 144 | 4.86 | 65; 101; 144 | 5.49 | 77; 138; 149 | 7.43 | 10; 101; 138 | 5.82 | 5; 122; 145 | 9.30 |
| 73; 90; 127 | 2.04 | 68; 82; 144 | 4.86 | 76; 101; 145 | 5.49 | 80; 149; 150 | 7.43 | 53; 85; 101 | 5.82 | 72; 113; 144 | 9.30 |
| 83; 103; 127 | 2.04 | 68; 83; 144 | 4.86 | 61; 78; 144 | 5.49 | 90; 133; 149 | 7.43 | 92; 101; 126 | 5.82 | 85; 121; 149 | 9.30 |
| 80; 91; 110 | 2.04 | 68; 84; 144 | 4.86 | 85; 101; 145 | 5.49 | 10; 77; 114 | 7.43 | 81; 90; 101 | 5.82 | 72; 122; 141 | 9.30 |
| 13; 110; 127 | 2.04 | 68; 85; 144 | 4.86 | 65; 97; 144 | 5.49 | 90; 114; 129 | 7.43 | 1; 73; 101 | 5.82 | 12; 122; 144 | 9.30 |
| 28; 103; 127 | 2.04 | 54; 68; 144 | 4.86 | 17; 134; 145 | 5.49 | 78; 146; 149 | 7.43 | 8; 20; 101 | 5.82 | 107; 121; 142 | 9.30 |
| 45; 74; 118 | 2.04 | 69; 79; 144 | 4.86 | 65; 95; 145 | 5.49 | 8; 77; 138 | 7.43 | 67; 88; 101 | 5.82 | 22; 121; 142 | 9.30 |
| 7; 44; 127 | 2.04 | 8; 68; 144 | 4.86 | 61; 82; 145 | 5.49 | 89; 114; 145 | 7.43 | 47; 69; 101 | 5.82 | 61; 122; 146 | 9.30 |
| 44; 62; 127 | 2.04 | 54; 70; 144 | 4.86 | 17; 119; 145 | 5.49 | 8; 138; 149 | 7.43 | 22; 83; 101 | 5.82 | 61; 122; 145 | 9.30 |
| 6; 108; 127 | 2.04 | 54; 72; 144 | 4.86 | 61; 87; 144 | 5.49 | 120; 124; 141 | 7.43 | 44; 92; 120 | 5.82 | 61; 122; 144 | 9.30 |
| 45; 72; 118 | 2.04 | 70; 108; 144 | 4.86 | 37; 101; 145 | 5.49 | 89; 148; 149 | 7.43 | 61; 63; 101 | 5.82 | 61; 122; 141 | 9.30 |
| 74; 90; 127 | 2.04 | 120; 144; 149 | 4.86 | 28; 94; 141 | 5.49 | 89; 149; 150 | 7.43 | 10; 100; 101 | 5.82 | 122; 124; 145 | 9.30 |
| 33; 72; 147 | 2.04 | 120; 144; 146 | 4.86 | 17; 101; 145 | 5.49 | 80; 84; 145 | 7.43 | 52; 82; 101 | 5.82 | 36; 113; 144 | 9.30 |
| 9; 127; 142 | 2.04 | 121; 126; 144 | 4.86 | 28; 94; 149 | 5.49 | 84; 133; 149 | 7.43 | 6; 92; 101 | 5.82 | 82; 122; 141 | 9.30 |
| 45; 70; 118 | 2.04 | 70; 114; 144 | 4.86 | 84; 132; 149 | 5.49 | 80; 145; 147 | 7.43 | 22; 84; 101 | 5.82 | 33; 121; 142 | 9.30 |
| 9; 127; 141 | 2.04 | 70; 119; 144 | 4.86 | 10; 66; 145 | 5.49 | 80; 88; 149 | 7.43 | 10; 67; 101 | 5.82 | 73; 122; 145 | 9.30 |

| Squamous epithelial cells | | Stellate cells | | Stromal cells | | Sympathoblasts | | Trophoblast giant cells | | Unipolar brush cells | |
|---|---|---|---|---|---|---|---|---|---|---|---|
| Clusters | Score | Clusters | Score | Clusters | Score | Clusters | Score | Clusters | Score | Clusters | Score |
| 6; 31; 109 | 7.41 | 72; 116; 120 | 9.48 | 38; 72; 121 | 11.03 | 26; 101; 104 | 4.75 | 94; 109; 123 | 5.79 | 3; 39; 99 | 6.17 |
| 31; 109; 140 | 7.41 | 9; 116; 122 | 9.48 | 23; 121; 138 | 11.03 | 62; 101; 104 | 3.60 | 20; 57; 114 | 5.79 | 12; 66; 100 | 5.18 |
| 31; 61; 109 | 7.41 | 6; 116; 120 | 9.48 | 26; 121; 145 | 11.03 | 74; 101; 104 | 3.60 | 10; 25; 109 | 5.79 | 15; 52; 100 | 5.18 |
| 31; 63; 109 | 7.41 | 60; 116; 122 | 9.48 | 72; 82; 121 | 11.03 | 3; 100; 148 | 3.60 | 64; 109; 132 | 5.79 | 1; 84; 98 | 5.18 |
| 6; 82; 109 | 6.03 | 52; 122; 129 | 9.48 | 121; 123; 126 | 11.03 | 32; 101; 108 | 3.60 | 10; 53; 109 | 5.79 | 15; 23; 99 | 5.18 |
| 6; 109; 129 | 6.03 | 10; 116; 121 | 8.32 | 86; 121; 150 | 11.03 | 101; 104; 108 | 3.60 | 66; 109; 132 | 5.79 | 15; 64; 100 | 5.18 |
| 6; 109; 131 | 6.03 | 49; 117; 122 | 8.32 | 82; 121; 123 | 11.03 | 25; 44; 101 | 3.60 | 14; 109; 123 | 5.79 | 15; 64; 99 | 5.18 |
| 7; 8; 109 | 6.03 | 10; 16; 122 | 8.32 | 10; 121; 138 | 11.03 | 32; 38; 101 | 3.60 | 64; 71; 109 | 5.79 | 2; 26; 119 | 5.18 |
| 55; 78; 109 | 6.03 | 8; 116; 122 | 8.32 | 72; 121; 123 | 11.03 | 42; 97; 100 | 3.60 | 62; 71; 109 | 5.79 | 26; 96; 99 | 5.18 |
| 31; 70; 109 | 6.03 | 19; 117; 122 | 8.32 | 38; 85; 121 | 11.03 | 29; 101; 129 | 3.60 | 54; 69; 109 | 4.57 | 12; 26; 37 | 5.18 |
| 31; 82; 109 | 6.03 | 10; 116; 122 | 8.32 | 121; 144; 150 | 9.99 | 81; 101; 104 | 3.60 | 20; 25; 148 | 4.57 | 15; 96; 99 | 5.18 |
| 26; 34; 109 | 6.03 | 10; 116; 120 | 8.32 | 75; 121; 150 | 9.99 | 3; 100; 129 | 3.60 | 20; 25; 147 | 4.57 | 26; 99; 147 | 5.18 |
| 26; 62; 109 | 6.03 | 38; 63; 121 | 8.32 | 54; 121; 149 | 9.99 | 42; 84; 101 | 3.60 | 20; 25; 146 | 4.57 | 12; 63; 100 | 5.18 |
| 25; 75; 109 | 6.03 | 29; 116; 120 | 8.32 | 113; 121; 134 | 9.99 | 19; 97; 146 | 3.60 | 20; 25; 142 | 4.57 | 26; 81; 99 | 5.18 |
| 29; 109; 133 | 6.03 | 7; 116; 120 | 8.32 | 54; 121; 150 | 9.99 | 61; 101; 104 | 3.60 | 64; 123; 142 | 4.57 | 1; 56; 98 | 5.18 |
| 26; 30; 109 | 6.03 | 36; 116; 120 | 8.32 | 9; 121; 138 | 9.99 | 3; 64; 100 | 3.60 | 20; 25; 149 | 4.57 | 4; 26; 81 | 5.18 |
| 34; 75; 109 | 6.03 | 52; 122; 142 | 8.32 | 9; 121; 145 | 9.99 | 63; 72; 101 | 3.60 | 28; 31; 63 | 4.57 | 1; 83; 98 | 5.18 |
| 8; 82; 109 | 6.03 | 116; 120; 129 | 8.32 | 5; 121; 124 | 9.99 | 29; 94; 101 | 3.60 | 62; 109; 123 | 4.57 | 15; 65; 99 | 5.18 |
| 6; 7; 109 | 6.03 | 106; 116; 122 | 8.32 | 5; 121; 129 | 9.99 | 101; 104; 114 | 3.60 | 62; 109; 118 | 4.57 | 1; 98; 148 | 5.18 |
| 55; 109; 131 | 6.03 | 52; 120; 122 | 8.32 | 61; 72; 121 | 9.99 | 7; 88; 101 | 3.60 | 25; 57; 109 | 4.57 | 2; 27; 100 | 5.18 |
| 64; 99; 109 | 6.03 | 10; 122; 129 | 8.32 | 5; 121; 138 | 9.99 | 3; 33; 100 | 3.60 | 29; 109; 123 | 4.57 | 26; 77; 99 | 5.18 |
| 8; 108; 109 | 6.03 | 52; 118; 122 | 8.32 | 113; 121; 144 | 9.99 | 3; 84; 100 | 3.60 | 11; 109; 123 | 4.57 | 1; 98; 147 | 5.18 |
| 34; 109; 131 | 6.03 | 5; 116; 121 | 8.32 | 10; 121; 123 | 9.99 | 53; 63; 101 | 3.60 | 31; 70; 120 | 4.57 | 1; 79; 98 | 5.18 |
| 6; 26; 109 | 6.03 | 44; 116; 122 | 8.32 | 38; 79; 121 | 9.99 | 3; 28; 100 | 3.60 | 31; 71; 79 | 4.57 | 2; 26; 148 | 5.18 |
| 56; 109; 131 | 6.03 | 72; 122; 129 | 8.32 | 10; 90; 121 | 9.99 | 26; 32; 68 | 3.60 | 31; 71; 81 | 4.57 | 4; 15; 26 | 5.18 |
| 90; 109; 131 | 6.03 | 71; 72; 116 | 8.32 | 82; 121; 150 | 9.99 | 32; 85; 101 | 3.60 | 31; 71; 94 | 4.57 | 26; 95; 99 | 5.18 |
| 6; 88; 109 | 6.03 | 33; 116; 120 | 8.32 | 10; 121; 145 | 9.99 | 38; 101; 104 | 3.60 | 31; 71; 98 | 4.57 | 12; 75; 100 | 5.18 |
| 29; 62; 109 | 6.03 | 10; 71; 112 | 7.22 | 29; 121; 149 | 9.99 | 32; 76; 101 | 3.60 | 54; 66; 109 | 4.57 | 6; 26; 99 | 5.18 |
| 6; 10; 109 | 6.03 | 48; 121; 132 | 7.22 | 72; 90; 121 | 9.99 | 34; 94; 102 | 3.60 | 17; 31; 71 | 4.57 | 4; 26; 37 | 5.18 |
| 6; 42; 109 | 6.03 | 48; 121; 129 | 7.22 | 101; 123; 144 | 9.99 | 101; 104; 129 | 3.60 | 54; 66; 123 | 4.57 | 39; 100; 142 | 5.18 |
| 34; 79; 109 | 6.03 | 10; 71; 117 | 7.22 | 53; 121; 129 | 9.99 | 68; 101; 104 | 3.60 | 55; 109; 123 | 4.57 | 2; 26; 81 | 5.18 |
| 8; 59; 109 | 6.03 | 55; 120; 122 | 7.22 | 54; 108; 121 | 9.99 | 26; 32; 92 | 3.60 | 38; 64; 109 | 4.57 | 4; 15; 62 | 5.18 |
| 31; 62; 109 | 6.03 | 13; 121; 122 | 7.22 | 72; 121; 126 | 9.99 | 70; 101; 104 | 3.60 | 57; 80; 109 | 4.57 | 3; 95; 99 | 5.18 |
| 6; 33; 109 | 6.03 | 5; 120; 122 | 7.22 | 61; 108; 121 | 9.99 | 32; 44; 102 | 3.60 | 8; 109; 123 | 4.57 | 26; 94; 99 | 5.18 |
| 29; 66; 109 | 6.03 | 27; 121; 122 | 7.22 | 85; 121; 123 | 9.99 | 32; 100; 104 | 3.60 | 10; 63; 109 | 4.57 | 27; 100; 141 | 5.18 |
| 64; 109; 131 | 6.03 | 120; 122; 129 | 7.22 | 38; 83; 121 | 9.99 | 101; 104; 126 | 3.60 | 17; 20; 31 | 4.57 | 15; 80; 98 | 5.18 |
| 29; 109; 150 | 6.03 | 55; 122; 138 | 7.22 | 29; 121; 138 | 9.99 | 39; 94; 101 | 3.60 | 10; 64; 109 | 4.57 | 2; 39; 99 | 5.18 |
| 8; 33; 109 | 6.03 | 13; 63; 121 | 7.22 | 23; 121; 144 | 9.99 | 29; 101; 146 | 3.60 | 65; 109; 123 | 4.57 | 39; 100; 141 | 5.18 |
| 1; 31; 109 | 6.03 | 52; 83; 122 | 7.22 | 75; 121; 123 | 9.99 | 32; 94; 101 | 3.60 | 66; 75; 109 | 4.57 | 15; 26; 99 | 5.18 |
| 31; 44; 109 | 6.03 | 55; 122; 129 | 7.22 | 29; 121; 123 | 9.99 | 63; 94; 101 | 3.60 | 68; 109; 120 | 4.57 | 12; 54; 100 | 5.18 |
| 31; 109; 150 | 6.03 | 10; 122; 146 | 7.22 | 122; 123; 129 | 9.99 | 32; 88; 101 | 3.60 | 10; 66; 109 | 4.57 | 2; 100; 116 | 5.18 |
| 78; 109; 131 | 6.03 | 116; 122; 142 | 7.22 | 48; 121; 148 | 9.99 | 3; 100; 101 | 3.60 | 68; 109; 132 | 4.57 | 2; 100; 117 | 5.18 |
| 67; 109; 131 | 6.03 | 9; 117; 122 | 7.22 | 79; 121; 150 | 9.99 | 7; 32; 101 | 3.60 | 28; 31; 84 | 4.57 | 23; 100; 141 | 5.18 |
| 70; 109; 131 | 6.03 | 54; 118; 122 | 7.22 | 5; 108; 121 | 9.99 | 32; 101; 126 | 3.60 | 10; 69; 109 | 4.57 | 39; 100; 145 | 5.18 |
| 109; 131; 150 | 6.03 | 117; 120; 129 | 7.22 | 10; 121; 149 | 9.99 | 7; 94; 101 | 3.60 | 38; 66; 109 | 4.57 | 3; 15; 99 | 5.18 |
| 31; 72; 109 | 6.03 | 8; 90; 121 | 7.22 | 54; 121; 138 | 9.99 | 28; 101; 104 | 3.60 | 84; 94; 150 | 4.57 | 15; 28; 99 | 5.18 |
| 74; 109; 131 | 6.03 | 86; 116; 122 | 7.22 | 30; 95; 121 | 9.99 | 57; 101; 104 | 3.60 | 68; 94; 109 | 4.57 | 39; 93; 100 | 4.25 |
| 31; 66; 109 | 6.03 | 5; 122; 146 | 7.22 | 93; 121; 150 | 9.99 | 32; 101; 129 | 3.60 | 31; 90; 134 | 4.57 | 1; 68; 99 | 4.25 |
| 28; 55; 109 | 6.03 | 13; 38; 121 | 7.22 | 26; 121; 150 | 9.99 | 42; 101; 129 | 3.60 | 31; 90; 132 | 4.57 | 1; 68; 98 | 4.25 |
| 7; 41; 109 | 6.03 | 50; 120; 122 | 7.22 | 68; 72; 121 | 9.99 | 32; 101; 132 | 3.60 | 17; 25; 31 | 4.57 | 2; 26; 39 | 4.25 |
| 25; 31; 109 | 6.03 | 38; 120; 121 | 7.22 | 90; 121; 123 | 9.99 | 32; 42; 101 | 3.60 | 20; 25; 31 | 4.57 | 26; 79; 99 | 4.25 |
| 6; 84; 109 | 6.03 | 117; 121; 122 | 7.22 | 84; 121; 150 | 9.99 | 77; 101; 104 | 3.60 | 98; 109; 123 | 4.57 | 2; 26; 149 | 4.25 |
| 6; 94; 109 | 6.03 | 71; 85; 116 | 7.22 | 38; 69; 121 | 9.99 | 32; 33; 101 | 3.60 | 31; 84; 120 | 4.57 | 2; 26; 95 | 4.25 |
| 34; 63; 109 | 6.03 | 7; 122; 129 | 7.22 | 5; 26; 121 | 9.99 | 1; 101; 104 | 3.60 | 27; 71; 109 | 4.57 | 15; 61; 99 | 4.25 |
| 8; 109; 131 | 6.03 | 2; 121; 122 | 7.22 | 120; 123; 145 | 9.99 | 42; 101; 149 | 3.60 | 31; 61; 90 | 4.57 | 1; 68; 78 | 4.25 |
| 6; 14; 109 | 6.03 | 20; 116; 120 | 7.22 | 120; 123; 144 | 9.99 | 61; 72; 101 | 3.60 | 31; 61; 94 | 4.57 | 2; 26; 37 | 4.25 |
| 6; 68; 109 | 6.03 | 20; 116; 122 | 7.22 | 26; 63; 121 | 9.99 | 3; 100; 104 | 3.60 | 31; 62; 71 | 4.57 | 26; 49; 81 | 4.25 |
| 34; 72; 109 | 6.03 | 86; 122; 129 | 7.22 | 68; 121; 150 | 9.99 | 69; 101; 104 | 3.60 | 20; 30; 109 | 4.57 | 15; 83; 99 | 4.25 |
| 6; 57; 109 | 6.03 | 10; 73; 122 | 7.22 | 51; 121; 138 | 9.99 | 3; 70; 100 | 3.60 | 63; 109; 149 | 4.57 | 37; 38; 99 | 4.25 |
| 4; 64; 109 | 6.03 | 60; 104; 122 | 7.22 | 26; 121; 138 | 9.99 | 32; 82; 101 | 3.60 | 10; 31; 109 | 4.57 | 56; 100; 141 | 4.25 |
| 7; 31; 109 | 6.03 | 48; 121; 122 | 7.22 | 90; 121; 150 | 9.99 | 39; 84; 101 | 3.60 | 10; 31; 123 | 4.57 | 63; 100; 141 | 4.25 |
| 29; 75; 109 | 6.03 | 117; 120; 122 | 7.22 | 72; 121; 145 | 9.99 | 34; 100; 104 | 3.60 | 10; 31; 130 | 4.57 | 39; 80; 100 | 4.25 |
| 65; 109; 131 | 6.03 | 52; 61; 122 | 7.22 | 70; 72; 121 | 9.99 | 64; 101; 104 | 3.60 | 57; 69; 109 | 4.57 | 73; 100; 141 | 4.25 |
| 29; 34; 109 | 6.03 | 10; 71; 122 | 7.22 | 29; 83; 121 | 9.99 | 3; 25; 100 | 3.60 | 45; 61; 109 | 4.57 | 24; 100; 118 | 4.25 |
| 6; 63; 109 | 6.03 | 55; 118; 122 | 7.22 | 72; 121; 138 | 9.99 | 25; 101; 129 | 3.60 | 10; 31; 131 | 4.57 | 15; 99; 100 | 4.25 |
| 25; 109; 150 | 6.03 | 52; 117; 122 | 7.22 | 108; 121; 129 | 9.99 | 25; 61; 101 | 3.60 | 17; 76; 109 | 4.57 | 2; 26; 138 | 4.25 |
| 8; 31; 109 | 6.03 | 38; 86; 121 | 7.22 | 108; 121; 145 | 9.99 | 26; 101; 129 | 3.60 | 67; 109; 123 | 4.57 | 2; 26; 108 | 4.25 |
| 5; 31; 109 | 6.03 | 10; 72; 122 | 7.22 | 108; 121; 150 | 9.99 | 32; 101; 150 | 3.60 | 57; 70; 109 | 4.57 | 39; 52; 99 | 4.25 |
| 8; 74; 109 | 6.03 | 72; 122; 135 | 7.22 | 26; 121; 135 | 9.99 | 25; 101; 104 | 3.60 | 31; 63; 120 | 4.57 | 19; 51; 100 | 4.25 |
| 31; 79; 109 | 6.03 | 55; 117; 122 | 7.22 | 76; 121; 150 | 9.99 | 3; 67; 100 | 3.60 | 10; 31; 150 | 4.57 | 15; 52; 96 | 4.25 |
| 7; 82; 109 | 6.03 | 55; 116; 122 | 7.22 | 72; 121; 150 | 9.99 | 26; 94; 101 | 3.60 | 57; 66; 109 | 4.57 | 76; 100; 141 | 4.25 |
| 8; 61; 109 | 6.03 | 44; 116; 120 | 7.22 | 84; 121; 129 | 9.99 | 82; 101; 104 | 3.60 | 10; 31; 70 | 4.57 | 1; 69; 99 | 4.25 |
| 25; 34; 109 | 6.03 | 10; 72; 121 | 7.22 | 5; 121; 144 | 9.99 | 32; 101; 118 | 3.60 | 31; 57; 123 | 4.57 | 15; 29; 78 | 4.25 |
| 26; 31; 109 | 6.03 | 85; 116; 122 | 7.22 | 10; 72; 121 | 9.99 | 43; 97; 100 | 3.60 | 10; 31; 68 | 4.57 | 2; 26; 126 | 4.25 |
| 31; 83; 109 | 6.03 | 33; 86; 122 | 7.22 | 72; 73; 121 | 9.99 | 3; 86; 100 | 3.60 | 66; 84; 109 | 4.57 | 15; 98; 100 | 4.25 |
| 6; 41; 109 | 6.03 | 30; 118; 122 | 7.22 | 72; 75; 121 | 9.99 | 94; 101; 104 | 3.60 | 9; 31; 109 | 4.57 | 2; 26; 73 | 4.25 |
| 8; 63; 109 | 6.03 | 116; 121; 122 | 7.22 | 78; 121; 123 | 9.99 | 3; 69; 100 | 3.60 | 20; 31; 78 | 4.57 | 2; 26; 83 | 4.25 |
| 6; 8; 109 | 6.03 | 51; 94; 121 | 7.22 | 6; 121; 138 | 9.99 | 7; 101; 129 | 3.60 | 20; 31; 71 | 4.57 | 8; 99; 118 | 4.25 |
| 25; 29; 109 | 6.03 | 38; 75; 121 | 7.22 | 26; 38; 121 | 9.99 | 6; 32; 101 | 3.60 | 67; 89; 109 | 4.57 | 100; 135; 141 | 4.25 |
| 70; 99; 109 | 6.03 | 53; 117; 120 | 7.22 | 10; 121; 129 | 9.99 | 84; 101; 104 | 3.60 | 10; 30; 109 | 4.57 | 84; 100; 141 | 4.25 |
| 6; 36; 109 | 6.03 | 54; 122; 129 | 7.22 | 6; 121; 123 | 9.99 | 33; 61; 101 | 3.60 | 20; 94; 149 | 4.57 | 23; 43; 100 | 4.25 |
| 8; 41; 109 | 6.03 | 30; 116; 120 | 7.22 | 4; 121; 144 | 9.99 | 4; 100; 104 | 3.60 | 31; 57; 109 | 4.57 | 26; 79; 100 | 4.25 |
| 28; 31; 109 | 6.03 | 90; 121; 122 | 7.22 | 83; 121; 150 | 9.99 | 32; 35; 101 | 3.60 | 10; 31; 64 | 4.57 | 4; 26; 119 | 4.25 |
| 7; 74; 109 | 6.03 | 20; 95; 122 | 7.22 | 63; 121; 138 | 9.99 | 3; 63; 100 | 3.60 | 10; 31; 66 | 4.57 | 2; 26; 88 | 4.25 |
| 31; 84; 109 | 6.03 | 16; 30; 122 | 7.22 | 71; 123; 144 | 9.99 | 101; 104; 149 | 3.60 | 54; 61; 109 | 4.57 | 37; 56; 99 | 4.25 |
| 29; 55; 109 | 6.03 | 10; 61; 122 | 7.22 | 83; 121; 129 | 9.99 | 7; 25; 101 | 3.60 | 57; 71; 109 | 4.57 | 6; 12; 100 | 4.25 |
| 34; 84; 109 | 6.03 | 113; 116; 122 | 7.22 | 38; 53; 121 | 9.99 | 100; 101; 104 | 3.60 | 31; 66; 132 | 4.57 | 2; 15; 26 | 4.25 |
| 8; 42; 109 | 6.03 | 56; 116; 122 | 7.22 | 56; 83; 121 | 9.99 | 32; 72; 101 | 3.60 | 31; 66; 134 | 4.57 | 12; 62; 100 | 4.25 |
| 8; 99; 109 | 6.03 | 37; 90; 121 | 7.22 | 36; 121; 144 | 9.99 | 11; 100; 104 | 3.60 | 63; 79; 109 | 4.57 | 15; 57; 99 | 4.25 |
| 10; 31; 141 | 6.03 | 20; 72; 122 | 7.22 | 5; 29; 121 | 9.99 | 61; 94; 101 | 3.60 | 3; 109; 123 | 4.57 | 12; 87; 100 | 4.25 |
| 31; 69; 109 | 6.03 | 67; 116; 122 | 7.22 | 63; 108; 121 | 9.99 | 26; 63; 77 | 2.56 | 77; 109; 123 | 4.57 | 26; 98; 100 | 4.25 |
| 10; 31; 114 | 6.03 | 56; 116; 120 | 7.22 | 53; 121; 138 | 9.99 | 86; 88; 101 | 2.56 | 68; 71; 109 | 4.57 | 15; 69; 99 | 4.25 |
| 55; 109; 150 | 6.03 | 10; 63; 121 | 7.22 | 29; 72; 121 | 9.99 | 54; 87; 101 | 2.56 | 63; 71; 109 | 4.57 | 86; 100; 141 | 4.25 |
| 31; 75; 109 | 6.03 | 116; 122; 129 | 7.22 | 121; 129; 150 | 9.99 | 34; 100; 146 | 2.56 | 63; 68; 109 | 4.57 | 4; 39; 99 | 4.25 |
| 25; 66; 109 | 6.03 | 86; 94; 121 | 7.22 | 73; 121; 123 | 9.99 | 3; 77; 100 | 2.56 | 63; 66; 109 | 4.57 | 26; 82; 99 | 4.25 |
| 26; 69; 109 | 6.03 | 10; 33; 122 | 7.22 | 121; 123; 135 | 9.99 | 12; 32; 147 | 2.56 | 48; 109; 123 | 4.57 | 15; 84; 99 | 4.25 |
| 62; 109; 131 | 6.03 | 10; 92; 121 | 7.22 | 72; 84; 121 | 9.99 | 53; 94; 101 | 2.56 | 31; 68; 71 | 4.57 | 1; 63; 98 | 4.25 |
| 10; 31; 109 | 6.03 | 61; 94; 121 | 7.22 | 38; 90; 121 | 9.99 | 77; 88; 101 | 2.56 | 31; 68; 120 | 4.57 | 2; 15; 64 | 4.25 |
| 6; 74; 109 | 6.03 | 116; 120; 122 | 7.22 | 121; 123; 129 | 9.99 | 77; 100; 104 | 2.56 | 66; 79; 109 | 4.57 | 100; 141; 147 | 4.25 |
| 31; 70; 77 | 6.03 | 120; 122; 135 | 7.22 | 72; 83; 121 | 9.99 | 32; 68; 116 | 2.56 | 31; 94; 123 | 4.57 | 4; 26; 70 | 4.25 |

| Ureteric bud cells | | Vascular endothelial cells | | Visceral neurons | |
|---|---|---|---|---|---|
| Clusters | Score | Clusters | Score | Clusters | Score |
| 25; 34; 147 | 6.62 | 69; 124; 132 | 34.04 | 26; 68; 82 | 5.79 |
| 34; 35; 81 | 6.62 | 29; 124; 150 | 34.04 | 26; 75; 101 | 5.79 |
| 5; 106; 110 | 6.62 | 53; 81; 124 | 34.04 | 26; 63; 147 | 5.79 |
| 6; 107; 147 | 5.38 | 51; 124; 148 | 32.44 | 5; 100; 119 | 5.79 |
| 15; 106; 108 | 5.38 | 70; 74; 124 | 32.44 | 35; 44; 100 | 5.79 |
| 15; 35; 106 | 5.38 | 72; 124; 150 | 32.44 | 5; 26; 101 | 5.79 |
| 105; 106; 110 | 5.38 | 72; 81; 124 | 32.44 | 5; 97; 100 | 5.79 |
| 34; 35; 147 | 5.38 | 61; 72; 124 | 32.44 | 26; 68; 75 | 5.79 |
| 74; 107; 109 | 5.38 | 124; 142; 149 | 32.44 | 26; 63; 101 | 5.79 |
| 34; 35; 150 | 5.38 | 10; 83; 124 | 32.44 | 62; 100; 119 | 5.79 |
| 25; 74; 147 | 5.38 | 53; 124; 132 | 32.44 | 26; 68; 83 | 5.79 |
| 18; 107; 114 | 5.38 | 70; 75; 124 | 32.44 | 26; 63; 68 | 5.79 |
| 8; 106; 110 | 5.38 | 75; 124; 150 | 32.44 | 63; 72; 101 | 4.57 |
| 15; 62; 106 | 5.38 | 37; 61; 124 | 32.44 | 5; 82; 100 | 4.57 |
| 15; 84; 106 | 5.38 | 29; 37; 124 | 32.44 | 62; 70; 100 | 4.57 |
| 15; 70; 106 | 5.38 | 5; 81; 124 | 32.44 | 26; 84; 101 | 4.57 |
| 25; 33; 147 | 5.38 | 26; 124; 150 | 32.44 | 29; 68; 101 | 4.57 |
| 85; 107; 147 | 5.38 | 64; 124; 150 | 32.44 | 35; 68; 100 | 4.57 |
| 30; 32; 109 | 5.38 | 29; 124; 149 | 32.44 | 5; 83; 100 | 4.57 |
| 19; 106; 110 | 5.38 | 36; 124; 135 | 32.44 | 26; 70; 101 | 4.57 |
| 6; 15; 106 | 5.38 | 53; 61; 124 | 32.44 | 62; 83; 100 | 4.57 |
| 15; 69; 106 | 5.38 | 44; 75; 124 | 32.44 | 26; 82; 101 | 4.57 |
| 25; 75; 147 | 5.38 | 65; 72; 124 | 32.44 | 44; 53; 100 | 4.57 |
| 16; 107; 109 | 5.38 | 53; 68; 124 | 30.87 | 62; 100; 147 | 4.57 |
| 25; 35; 147 | 5.38 | 29; 38; 124 | 30.87 | 6; 62; 100 | 4.57 |
| 15; 74; 106 | 5.38 | 10; 84; 124 | 30.87 | 9; 100; 119 | 4.57 |
| 35; 81; 107 | 5.38 | 70; 124; 132 | 30.87 | 62; 64; 100 | 4.57 |
| 15; 18; 106 | 5.38 | 53; 65; 124 | 30.87 | 64; 92; 101 | 4.57 |
| 15; 85; 106 | 5.38 | 60; 124; 148 | 30.87 | 62; 82; 100 | 4.57 |
| 15; 81; 106 | 5.38 | 69; 72; 124 | 30.87 | 62; 100; 126 | 4.57 |
| 19; 85; 109 | 5.38 | 53; 124; 148 | 30.87 | 26; 68; 101 | 4.57 |
| 9; 34; 109 | 5.38 | 72; 124; 146 | 30.87 | 35; 87; 100 | 4.57 |
| 74; 81; 107 | 5.38 | 70; 124; 126 | 30.87 | 65; 100; 119 | 4.57 |
| 25; 147; 150 | 5.38 | 6; 72; 124 | 30.87 | 5; 10; 100 | 4.57 |
| 15; 83; 106 | 5.38 | 72; 75; 124 | 30.87 | 9; 100; 118 | 4.57 |
| 15; 79; 106 | 5.38 | 37; 79; 124 | 30.87 | 64; 97; 100 | 4.57 |
| 35; 107; 140 | 5.38 | 54; 81; 124 | 30.87 | 44; 75; 100 | 4.57 |
| 15; 65; 106 | 5.38 | 54; 85; 124 | 30.87 | 5; 35; 100 | 4.57 |
| 15; 106; 150 | 5.38 | 37; 82; 124 | 30.87 | 6; 100; 147 | 4.57 |
| 19; 109; 147 | 5.38 | 37; 83; 124 | 30.87 | 26; 72; 101 | 4.57 |
| 15; 106; 149 | 5.38 | 44; 124; 150 | 30.87 | 35; 70; 100 | 4.57 |
| 15; 106; 147 | 5.38 | 53; 73; 124 | 30.87 | 26; 74; 101 | 4.57 |
| 15; 64; 106 | 5.38 | 72; 124; 132 | 30.87 | 5; 100; 118 | 4.57 |
| 84; 107; 147 | 5.38 | 72; 124; 134 | 30.87 | 35; 61; 100 | 4.57 |
| 19; 35; 109 | 5.38 | 53; 85; 124 | 30.87 | 38; 62; 100 | 4.57 |
| 75; 107; 147 | 5.38 | 53; 84; 124 | 30.87 | 5; 100; 112 | 4.57 |
| 34; 35; 85 | 5.38 | 53; 79; 124 | 30.87 | 5; 6; 100 | 4.57 |
| 7; 106; 110 | 4.23 | 79; 81; 124 | 30.87 | 5; 92; 100 | 4.57 |
| 32; 37; 109 | 4.23 | 29; 124; 132 | 30.87 | 5; 92; 101 | 4.57 |
| 73; 81; 106 | 4.23 | 68; 72; 124 | 30.87 | 62; 68; 100 | 4.57 |
| 5; 8; 110 | 4.23 | 70; 124; 146 | 30.87 | 44; 100; 119 | 4.57 |
| 34; 35; 108 | 4.23 | 51; 124; 134 | 30.87 | 64; 100; 119 | 4.57 |
| 25; 84; 147 | 4.23 | 36; 53; 124 | 30.87 | 63; 94; 101 | 4.57 |
| 31; 85; 112 | 4.23 | 64; 124; 126 | 30.87 | 5; 90; 100 | 4.57 |
| 19; 109; 148 | 4.23 | 29; 83; 124 | 30.87 | 62; 90; 100 | 4.57 |
| 88; 106; 110 | 4.23 | 51; 124; 146 | 30.87 | 44; 100; 147 | 4.57 |
| 29; 106; 110 | 4.23 | 26; 70; 124 | 30.87 | 10; 68; 100 | 4.57 |
| 32; 34; 109 | 4.23 | 51; 124; 149 | 30.87 | 62; 84; 100 | 4.57 |
| 70; 104; 109 | 4.23 | 29; 84; 124 | 30.87 | 26; 73; 101 | 4.57 |
| 32; 74; 109 | 4.23 | 18; 124; 133 | 30.87 | 5; 44; 100 | 4.57 |
| 35; 133; 147 | 4.23 | 64; 124; 146 | 30.87 | 5; 38; 100 | 4.57 |
| 25; 34; 35 | 4.23 | 5; 75; 124 | 30.87 | 44; 70; 100 | 4.57 |
| 106; 110; 125 | 4.23 | 18; 124; 142 | 30.87 | 87; 100; 147 | 4.57 |
| 25; 81; 106 | 4.23 | 17; 124; 135 | 30.87 | 5; 100; 147 | 4.57 |
| 6; 17; 110 | 4.23 | 75; 124; 134 | 30.87 | 10; 87; 100 | 4.57 |
| 18; 106; 110 | 4.23 | 26; 124; 134 | 30.87 | 64; 100; 147 | 4.57 |
| 35; 107; 148 | 4.23 | 37; 124; 149 | 30.87 | 63; 100; 119 | 4.57 |
| 70; 103; 107 | 4.23 | 37; 124; 146 | 30.87 | 5; 70; 100 | 4.57 |
| 32; 103; 109 | 4.23 | 37; 124; 134 | 30.87 | 35; 82; 100 | 4.57 |
| 14; 109; 114 | 4.23 | 64; 85; 124 | 30.87 | 5; 62; 100 | 4.57 |
| 15; 66; 106 | 4.23 | 29; 124; 126 | 30.87 | 9; 97; 100 | 4.57 |
| 19; 109; 146 | 4.23 | 70; 124; 150 | 30.87 | 62; 97; 100 | 4.57 |
| 28; 106; 110 | 4.23 | 75; 79; 124 | 30.87 | 65; 100; 147 | 4.57 |
| 37; 81; 106 | 4.23 | 64; 81; 124 | 30.87 | 10; 44; 100 | 4.57 |
| 11; 106; 115 | 4.23 | 33; 69; 124 | 30.87 | 6; 100; 119 | 4.57 |
| 34; 35; 88 | 4.23 | 36; 72; 124 | 30.87 | 63; 100; 147 | 4.57 |
| 106; 110; 126 | 4.23 | 35; 124; 142 | 30.87 | 64; 90; 100 | 4.57 |
| 106; 107; 110 | 4.23 | 82; 124; 150 | 30.87 | 44; 63; 100 | 4.57 |
| 16; 34; 87 | 4.23 | 68; 124; 132 | 30.87 | 26; 64; 101 | 4.57 |
| 5; 76; 110 | 4.23 | 37; 124; 126 | 30.87 | 44; 84; 100 | 4.57 |
| 54; 107; 109 | 4.23 | 22; 124; 145 | 30.87 | 10; 62; 100 | 4.57 |
| 5; 33; 110 | 4.23 | 64; 108; 124 | 30.87 | 26; 63; 119 | 4.57 |
| 15; 88; 106 | 4.23 | 96; 124; 135 | 30.87 | 6; 35; 100 | 4.57 |
| 34; 35; 93 | 4.23 | 26; 72; 124 | 30.87 | 63; 75; 101 | 4.57 |
| 35; 107; 142 | 4.23 | 37; 77; 124 | 30.87 | 44; 61; 100 | 4.57 |
| 106; 110; 148 | 4.23 | 53; 70; 124 | 30.87 | 44; 82; 100 | 4.57 |
| 47; 106; 110 | 4.23 | 70; 72; 124 | 30.87 | 5; 64; 100 | 4.57 |
| 15; 28; 106 | 4.23 | 53; 124; 147 | 30.87 | 26; 101; 143 | 4.57 |
| 106; 110; 150 | 4.23 | 72; 83; 124 | 30.87 | 35; 65; 100 | 4.57 |
| 105; 106; 109 | 4.23 | 72; 84; 124 | 30.87 | 5; 68; 100 | 4.57 |
| 5; 17; 110 | 4.23 | 37; 72; 124 | 30.87 | 62; 85; 100 | 4.57 |
| 106; 110; 147 | 4.23 | 86; 124; 150 | 30.87 | 10; 100; 119 | 4.57 |
| 19; 109; 150 | 4.23 | 72; 85; 124 | 30.87 | 26; 68; 72 | 4.57 |
| 86; 107; 109 | 4.23 | 72; 79; 124 | 30.87 | 26; 68; 73 | 4.57 |
| 74; 84; 107 | 4.23 | 7; 124; 150 | 30.87 | 100; 101; 119 | 4.57 |
| 15; 87; 106 | 4.23 | 75; 83; 124 | 30.87 | 73; 87; 100 | 4.57 |
| 62; 106; 110 | 4.23 | 124; 142; 150 | 30.87 | 44; 79; 100 | 4.57 |
| 77; 107; 109 | 4.23 | 36; 124; 150 | 30.87 | 44; 93; 100 | 4.57 |
| 106; 110; 132 | 4.23 | 37; 53; 124 | 30.87 | 39; 68; 84 | 3.45 |
| 9; 78; 109 | 4.23 | 35; 44; 124 | 30.87 | 25; 72; 100 | 3.45 |

Various modifications and variations of the described methods, pharmaceutical compositions, and kits of the invention can be apparent to those skilled in the art without departing from the scope and spirit of the invention. Although the invention has been described in connection with specific embodiments, it can be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of differentiating a pluripotent cell population to a target cell comprising overexpressing one or more transcription factors from Table 1 or Table 3 in a pluripotent cell population, and selecting cells expressing one or more target cell markers.

2. The method of claim 1, wherein the target cell is a neural progenitor and selecting cells comprises selecting cells expressing one or more radial glial cell markers.

3. The method of claim 2, wherein the one or more transcription factors are selected from the group consisting of RFX4, NFIB, ASCL1, PAX6, EOMES, FOS, OTX1, NFIC, LHX2, RCOR2, GLI3, NOTCH2, HELLS, BCL11A, HES1, FANCD2, SOX9, FEZF2, and TCF7L2.

4. The method of claim 3, wherein the one or more transcription factors are RFX4, NFIB, ASCL1, PAX6, or a combination thereof.

5. The method of any one of the preceding claims, wherein the one or more radial glial cell markers are selected from Table 2.

6. The method of claim 5, wherein the one or more radial glial cell markers are selected from the group consisting of NES, VIM, SLC1A3, and PAX6.

7. The method of any of the claims 2 to 6, wherein selecting further comprises selecting cells enriched for expression of one or more gene signatures expressed in in vivo radial glia cells.

8. The method of claim 7, wherein selecting cells enriched for expression of one or more gene signatures expressed in in vivo radial glia cells comprises:
a) identifying gene signatures for each TF by identifying differentially expressed genes between cells overexpressing a transcription factor and control cells; and
b) selecting cells having a signature that is enriched in an in vivo radial glia cell type.

9. An isolated neural progenitor cell produced by the method of any one of claims 2 to 8.

10. A therapeutic composition comprising the isolated neural progenitor cell of claim 9.

11. An ex vivo system comprising the isolated neural progenitor cell of claim 9.

12. A method of producing neurons, astrocytes and/or oligodendrocytes, comprising expressing one or more transcription factors from Table 1 in the isolated neural progenitor cell of claim 9 and inducing spontaneous differentiation of the isolated neural progenitor cells.

13. A method of producing neurons, astrocytes and/or oligodendrocytes comprising expressing one or more transcription factors from Table 1 in the isolated neural progenitor cell of claim 9 and inducing directed differentiation of the isolated neural progenitor cells.

14. An isolated neuron, astrocyte, or oligodendrocyte produced according to the method of claim 12 or 13.

15. A therapeutic composition comprising the isolated neuron, astrocyte, or oligodendrocyte of claim 14.

16. An ex vivo system comprising the isolated neurons, astrocytes, and/or oligodendrocytes of claim 14.

17. A non-naturally occurring population of stem cells comprising a reporter gene integrated into an endogenous locus of each stem cell in the population, wherein:
i. the endogenous locus is associated with a marker gene for a cell type of interest;
ii. the reporter gene is under control of the promoter for the marker gene; and
iii. the reporter gene and marker gene are expressed as separate proteins,
whereby the marker gene and reporter gene are co-expressed upon differentiation of the stem cells into the cell type of interest.

18. The non-naturally occurring population of stem cells of claim 17, further comprising a second reporter gene integrated into a second endogenous locus of the stem cell, wherein the locus is associated with a marker gene for a second cell type of interest, and wherein the second cell type of interest is more differentiated than the first cell type of interest.

19. The non-naturally occurring population of stem cells according to claim 17 or 18, wherein the reporter gene and marker gene are separated by a ribosomal skipping site.

20. The non-naturally occurring population of stem cells according to claim 19, wherein the ribosomal skipping site is a P2A sequence.

21. The non-naturally occurring population of stem cells according to any of claims 17 to 20, wherein the reporter gene is a fluorescent protein.

22. The non-naturally occurring population of stem cells according to any of claims 17 to 21, wherein the cell type of interest is a differentiated cell.

23. The non-naturally occurring population of stem cells according to any of claims 17 to 22, wherein the cell type of interest is a neural progenitor or mature neural cell type.

24. The non-naturally occurring population of stem cells according to claim 23, wherein the cell type of interest is a radial glia cell.

25. The non-naturally occurring population of stem cells according to claim 24, wherein the marker gene is selected from Table 2.

26. The non-naturally occurring population of stem cells of claim 25, wherein the marker gene is selected from the group consisting of NES, VIM, SLC1A3, and PAX6.

27. The non-naturally occurring population of stem cells according to claim 23, wherein the cell type of interest is an astrocyte.

28. The non-naturally occurring population of stem cells according to claim 27, wherein the marker gene is selected from the group consisting of ALDH1L1 and GFAP.

29. A pooled transcription factor screening system comprising:
i. a transcription factor library comprising one or more vectors encoding a transcription factor and a barcode identifying said transcription factor; and
ii. a population of pluripotent cells.

30. The system of claim 29, wherein the transcription factors encoded by the vectors are selected from Table 1 and/or Table 3.

31. The system of claim 29 or 30, wherein the population of pluripotent cells are stem cells according to any one of claims 17 to 28.

32. The system of claim 29 or 30, further comprising one or more fluorescent probes configured for detecting one or more target cell marker gene transcripts.

33. A method of screening for transcription factors capable of differentiating pluripotent cells into a cell type of interest, comprising:
a) introducing a transcription factor library comprising one or more vectors to a population of pluripotent cells, wherein each vector encodes:
i. a transcription factor selected from Table 1 and/or Table 3 or an agent capable of modulating said transcription factor, and
ii. a barcode identifying each transcription factor;
b) culturing the cells to allow differentiation of the cells;
c) selecting cells expressing one or more marker genes for the cell of interest; and
d) determining barcodes enriched in cells expressing the one or marker genes, thereby identifying transcription factors capable of differentiating pluripotent cells into a cell of interest.

34. The method of claim 33, wherein the population of pluripotent cells is a population of human embryonic stem cells (hESCs).

35. The method of claim 33 or 34, wherein selecting cells expressing one or more marker genes for the cell of interest comprises Flow-FISH using probes for the one or more marker genes.

36. The method of claim 33 or 34, wherein selecting cells expressing one or more marker genes for the cell of interest comprises single cell RNA-seq.

37. The method of claim 36, wherein selecting cells further comprises comparing single cell RNA-seq expression profiles of cells overexpressing one or more of the transcription factors to control cells to infer pseudotime for each cell, wherein transcription factors that increased pseudotimes direct differentiation.

38. The method of claim 36, wherein selecting cells further comprises grouping one or more of the transcription factors in modules that alter expression of the same gene programs, wherein transcription factors in the same modules are co-functional.

39. The method of claim 33, wherein the one or more populations of pluripotent cells are stem cells according to any of claims 17 to 28.

40. The method of claim 39, wherein selecting cells expressing one or marker genes for the cell of interest comprises detecting the reporter gene.

41. The method of any of claims 33 to 40, wherein each transcription factor is inducible.

42. The method of any of claims 33 to 41, wherein determining barcodes comprises sequencing the DNA barcode or transcript comprising the barcode.

43. The method of any of claims 33 to 42, wherein determining barcodes comprises amplification of barcode sequences.

44. The method of any of claims 41 to 43, wherein the method further comprises:
a) introducing the transcription factor library at a low cell density, such that the cells multiply into small colonies; and
b) inducing expression of the transcription factors or agents encoded by the vectors.

45. The method of any of claims 33 to 44, wherein the method further comprises introducing the vector library at a low MOI, such that most cells receive no more than one vector.

46. The method of any of claims 33 to 44, wherein the method further comprises introducing the vector library at a high MOI, such that most cells receive one or more vectors, whereby specific combinations of transcription factors capable of differentiating pluripotent cells into a cell type of interest are identified.

47. The method of any of claims 33 to 46, wherein the transcription factor library comprises viral vectors.

48. The method of claim 47, wherein the viral vectors are lentivirus, adenovirus or adeno associated virus (AAV) vectors.

49. The method of any of claims 33 to 48, wherein selecting cells comprises FACS.

50. The method of any of claims 33 to 49, wherein the transcription factor library further encodes a protein tag in frame with the transcription factor coding sequence.

51. The method of any of claims 33 to 49, wherein the population of stem cells expresses a CRISPR system and the transcription factor library comprises vectors encoding one or more CRISPR guide sequences targeting one of the transcription factors.

52. The method of claim 51, wherein the guide sequences comprise one or more aptamer sequences specific for binding an adaptor protein and the CRISPR system comprises an enzymatically inactive CRISPR enzyme and the adaptor protein comprising a functional domain.

53. The method of claim 51, wherein the CRISPR system comprises an enzymatically inactive CRISPR enzyme and a functional domain.

54. The method of claim 52 or 53, wherein the functional domain is a transcription activation or repression domain.

55. The method of any of claims 33 to 49, wherein the transcription factor library comprises vectors encoding a shRNA for one of the transcription factors.

56. The method of any of claims 33 to 55, wherein the transcription factors selected are normally expressed by the cell of interest.

57. The method of any of claims 33 to 56, wherein identifying transcription factors further comprises:
a) determining gene signatures for each identified TF, wherein the gene signature comprises differentially expressed genes between cells overexpressing each transcription factor and control cells; and
b) selecting transcription factors inducing a gene signature that is enriched in an in vivo cell type.

58. The method of claim 1, wherein the target cell is a cardiomyocyte, said method comprising overexpressing a transcription factor selected from the group consisting of MESP1, EOMES and ESR1 in a pluripotent cell population, and selecting cells expressing one or more cardiomyocyte markers.

59. The method of claim 58, wherein the transcription factor is EOMES.

60. The method of claim 59, wherein the amino acid sequence of EOMES is SEQ ID NO: 10807 or SEQ ID NO: 10808.

61. The method of any of claims 58 to 60, wherein the transcription factor is induced for about two days.

62. The method of any of claims 58 to 61, wherein the transcription factor is induced when the cell density is about 500,000 cells/ml.

63. The method of any of claims 58 to 62, wherein the one or more cardiomyocyte markers comprises TNNT2.

64. The method of any of claims 58 to 63, wherein selecting further comprises selecting cells enriched for expression of one or more gene signatures expressed in in vivo cardiomyocytes.

65. An isolated cardiomyocyte produced by the method of any one of claims 58 to 64.

66. A therapeutic composition comprising the isolated cardiomyocyte of claim 65.

67. An ex vivo system comprising the isolated cardiomyocyte of claim 65.

68. The method according to any of the preceding claims, wherein the pluripotent cell is an embryonic stem cell (ES) or induced pluripotent stem cell.

69. The method according to claim 68, wherein the stem cell is a human embryonic stem cell (ES).

70. The method according to claim 69, wherein the human embryonic stem cell is selected from the group consisting of HUES66, HUES64, HUES3, HUES8, HUES53, HUES28, HUES49, HUES9, HUES48, HUES45, HUES1, HUES44, HUES6, H1, HUES62, HUES65, H7, HUES13 and HUES63.

71. A stem cell comprising an exogenous nucleotide sequence capable of inducible expression of one or more transcription factors selected from the group consisting of RFX4, NFIB, ASCL1 and PAX6.

72. A stem cell comprising an exogenous nucleotide sequence capable of inducible expression of one or more transcription factors selected from the group consisting of MESP1, EOMES and ESR1.

73. The method of any of claims 2 to 8, further comprising inducing differentiation of the neural progenitors into neurons, astrocytes and/or oligodendrocytes.

74. The method of claim 73, wherein differentiation comprises spontaneous differentiation of the neural progenitors.

75. The method of claim 73, wherein differentiation comprises directed differentiation of the neural progenitors.

76. A method of predicting transcription factor combinations for differentiating a stem cell into a cell of interest comprising determining the average gene expression of one or more genes for two or more stem cells each expressing a single transcription factor and comparing the average expression to a gene signature specific for the cell of interest.

77. The method of claim 76, further comprising differentiating a stem cell into the cell of interest by expressing in the stem cell a double or triple combination of transcription factors whose average gene expression is most similar to a gene signature specific for the cell of interest.

78. A method of differentiating a stem cell into a cell of interest comprising expressing in the stem cell a double or triple combination of transcription factors selected from the clusters in Table 19.
